(19) United States
(12) Patent Application Publication
(10) Pub. No.: US 2026/0028354 A1
(43) Pub. Date: Jan. 29, 2026

(54) TETRAHYDRONAPHTHALENE DERIVATIVES AS ESTROGEN RECEPTOR DEGRADERS

(71) Applicants: REGENT OF THE UNIVERSITY OF MICHIGAN, Ann Arbor, MI (US); ONCOPIA THERAPEUTICS, INC. D/B/A/ SK LIFE SCIENCE LABS, King of Prussia, PA (US)

(72) Inventors: Guozhang Xu, New York, NY (US); Zhixiang Chen, Ann Arbor, MI (US); Rohan Rej, Ann Arbor, MI (US); Ranjan Kumar Acharyya, Ann Arbor, MI (US); Dimin Wu, Ann Arbor, MI (US); Ms. Mingliang Wang, Ann Arbor, MI (US); Biao Hu, Ann Arbor, MI (US); Jianfeng Lu, Ann Arbor, MI (US); Zhenwu Li, New York, NY (US); E. Scott Priestley, New York, NY (US); Shaomeng Wang, Superior Township, MI (US)

(21) Appl. No.: 18/992,004

(22) PCT Filed: Jul. 12, 2023
(86) PCT No.: PCT/US23/27439

§ 371 Date: Jan. 7, 2025

Related U.S. Application Data
(60) Provisional application No. 63/388,300, filed on Jul. 12, 2022.
(60) Provisional application No. 63/408,744, filed on Sep. 21, 2022.
(60) Provisional application No. 63/427,277, filed on Nov. 22, 2022.
(60) Provisional application No. 63/460,734, filed on Apr. 20, 2023.

(51) Int. Cl. C07D 519/00; A61K 31/4545; A61K 31/496; A61K 31/497; A61K 31/4985; A61K 31/506; A61K 31/5383; A61K 31/55; A61P 35/00; C07D 401/14; C07D 487/14; C07D 491/107; C07D 491/113; C07D 491/20; C07D 498/04; C07D 498/14

(52) U.S. Cl. CPC C07D 519/00; A61K 31/4545; A61K 31/496; A61K 31/497; A61K 31/4985; A61K 31/506; A61K 31/5383; A61K 31/55; A61P 35/00; C07D 401/14; C07D 487/14; C07D 491/107; C07D 491/113; C07D 491/20; C07D 498/04; C07D 498/14

(57) ABSTRACT

Described herein are compounds of Formula I and their pharmaceutically acceptable salts, solvates, or stereoisomers, as well as their uses (e.g., as estrogen receptor degraders).

### RELATED APPLICATIONS

This application claims the benefit of and priority to U.S. Provisional Application No. 63/388,300, filed Jul, 12, 2022; U.S. Provisional Application No, 63/408,744, filed September 21, 2022; U.S. Provisional Application No, 63/427,277, filed Nov., 22, 2022; and U.S. Provisional Application No, 63/460,734, filed Apr., 20, 2023; the contents of each of which are incorporated herein by reference in their entireties.

### BACKGROUND

Estrogen receptors (ERs) belong to the steroid/nuclear receptor superfamily involved in the regulation of eukaryotic gene expression, cellular proliferation, and differentiation in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (17B-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes. ERα is mainly expressed in reproductive tissues such as uterus, ovary, breast, bone, and white adipose tissue. It is well known that deregulation of ER signaling, specifically through ERα, results in uncontrolled cellular proliferation which eventually results into cancer. ER+breast cancer accounts for approximately 75% of all breast cancers diagnosed, as well as some ovarian and endometrial cancers.

Current therapy for ER+breast cancer including agents that inhibit the ER activity through direct binding to the ligand binding domain of the receptor (e.g., tamoxifen); blocking the synthesis of estrogen (e.g., aromatase inhibitor such as anastrozole and letrozole); or inducing the degradation of ER. Selective estrogen receptor degraders (SERD) are small molecules that target ERα for proteasome-dependent degradation. Fulvestrant is the only SERD that has been approved for the treatment of postmenopausal women with advanced ER+breast cancer with standard endocrine therapies. Because it has poor solubility and is not orally bioavailable, fulvestrant is administered clinically by a monthly intramuscular injection. To address the shortcomings of fulvestrant, oral bioavailable SERDs are being developed. However, the SERDs are only able to achieve partial degradation of the ER protein despite they are typically potent and effective in inducing degradation of ER protein in ER+breast cancer cells.

It is believed that ERα degradation may occur when both ERα and a ubiquitin ligase (e.g., cereblon E3 ligase (CRBN)) are bound and brought into close proximity for ubiquitination and subsequent degradation by proteasomes. A new approach would be to utilize the naturally occurring cellular ubiquitin-mediated degradation to develop a completely new class of therapeutics for the treatment of ER+metastatic breast cancer with nearly complete degradation of ER protein.

### SUMMARY

In certain aspects, the present disclosure provides compounds of Formula I:

T-L-C (I).

and pharmaceutically acceptable salts, solvates, or stereoisomers thereof, wherein:
C is of Formula I′-1

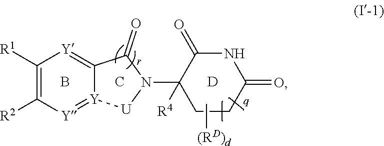

T is of Formula I-2:

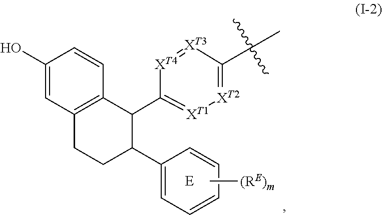

and
L is of Formula I′-3:

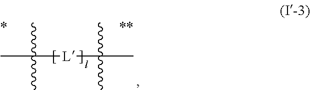

wherein each of the variables in Formulae I, I′-1, I-2, and I′-3, is described, embodied, and exemplified herein.

In certain aspects, the present disclosure provides pharmaceutical compositions comprising a compound disclosed herein, and a pharmaceutically acceptable excipient.

In certain aspects, the present disclosure provides methods of degrading an estrogen receptor in a subject, comprising administering to the subject a compound disclosed herein.

In certain aspects, the present disclosure provides uses of a compound disclosed herein in the manufacture of a medicament for degrading an estrogen receptor in a subject.

In certain aspects, the present disclosure provides compounds disclosed herein for use in degrading an estrogen receptor in a subject.

In certain aspects, the present disclosure provides methods of treating or preventing a disease or disorder in a subject in need thereof, comprising administering to the subject a compound disclosed herein (e.g., in a therapeutically effective amount).

In certain aspects, the present disclosure provides methods of treating a disease or disorder in a subject in need thereof, comprising administering to the subject a compound disclosed herein (e.g., in a therapeutically effective amount).

In certain aspects, the present disclosure provides uses of a compound disclosed herein in the manufacture of a medicament for treating or preventing a disease or disorder in a subject in need thereof.

In certain aspects, the present disclosure provides uses of a compound disclosed herein in the manufacture of a medicament for treating a disease or disorder in a subject in need thereof.

In certain aspects, the present disclosure provides compounds disclosed herein for use in treating or preventing a disease or disorder in a subject in need thereof.

In certain aspects, the present disclosure provides compounds disclosed herein for use in treating a disease or disorder in a subject in need thereof.

### DETAILED DESCRIPTION

The present disclosure relates to compounds and methods of degrading an estrogen receptor comprising contacting the estrogen receptor with a therapeutically effective amount of an estrogen receptor degrader disclosed herein. The present disclosure also relates to methods of treating an estrogen receptor-mediated disease or condition in a subject in need thereof by administering a therapeutically effective amount of an estrogen receptor degrader disclosed herein. The present disclosure further relates to methods of treating an estrogen receptor-mediated disease or condition in a subject in need thereof, comprising administering a pharmaceutical composition comprising a therapeutically effective amount of an estrogen receptor degrader disclosed herein.

### Compounds of the Application

In one aspect, the present disclosure provides compounds of Formula I:

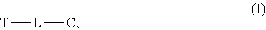

and pharmaceutically acceptable salts, solvates, or stereoisomers thereof,
wherein:
C is of Formula I′-1

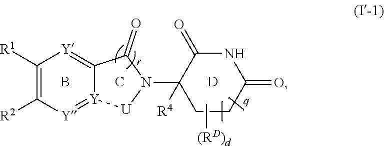

wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R² is *—Cy2—, wherein * denotes attachment to L;
—Cy2— is C₃₋₁₂ carbocyclylene or 3- to 12-membered heterocyclylene, wherein the carbocyclylene or heterocyclylene is optionally substituted with one or more R^{u}; or
R¹ and R², together with the intervening carbon atoms, form Ring A attached to L, wherein Ring A is optionally substituted C₃₋₁₂ carbocycle or 5- to 16-membered hetcrocycle;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form Ring A attached to L, wherein Ring

A is optionally substituted 5- to 16-membered heterocycle;
provided that R¹ and R², and R² and R³, do not both form Ring A attached to L;
Y′ is N or CR^{Y};
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{a};
[symbol] denotes an optional covalent bond between Y and U;
i) when the bond between Y and U is absent:
r is 0 or 1;
Y is N or CR^{Y};

R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
U is hydrogen or C₁₋₆ alkyl optionally substituted with one or more R^{u};
ii) when the bond between Y and U is present:
r is 1;
Y is C;
U is -CH₂-, —C(═O)—, —(C═O)-N(R^{U})-*, or —N═C(R^{U})-*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl; and
each R^{D} is independently oxo, halogen, —CN, —NO₂, —OH, —NH₂, C₁₋₆ alkyl, C₁₋₆ alkoxy, C₁₋₆ alkylamino, C₂₋₆ alkonyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
d is an integer from 0 to 4; and
q is an integer from 0 to 2,

T is of Formula I-2:

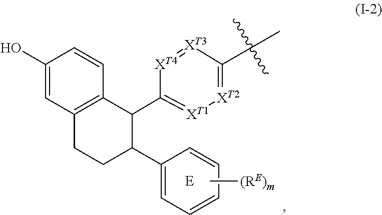

wherein:
each of X^{T1}, X^{T2}, X^{T3}, and X^{T4} is independently N or CRT;
each occurrence of R^{T} is independently hydrogen, halogen, —CN, —NO₂, —OH, —NH₂, C₁₋₆ alkyl, C₁₋₆-alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{E} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 5;
L is of Formula I′-3:

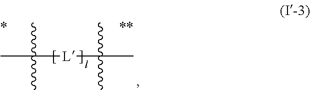

wherein:
* denotes attachment to T, and ** denotes attachment to C;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L})—, —C(═O)O—, —N(R^{L})—, —O—, —S—, or —S(═O)₂—, wherein the alkylene, heteroalkylene, alkenylene, alkynylene, carbocyclylene, heterocyclylene, arylene, or heteroarylene is optionally substituted with one or more R^{u};
each occurrence of R^{L} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and 1 is an integer selected from 0 to 10,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain aspects, the present disclosure provides compounds of Formula I:

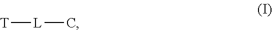

and pharmaceutically acceptable salts, solvates, or stereoisomers thereof,
wherein:
C is of Formula I-1

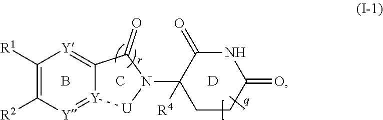

wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R² is *—Cy², wherein * denotes attachment to L;
—Cy²- is C₃₋₁₂ carbocyclylene or 3- to 12-membered heterocyclylene, wherein the carbocyclylene or heterocyclylene is optionally substituted with one or more R^{u}; or
R¹ and R², together with the intervening carbon atoms, form Ring A attached to L, wherein Ring A is optionally substituted C₃₋₁₂ carbocycle or 5- to 16-membered heterocycle;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(-O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form Ring A attached to L, wherein Ring A is optionally substituted 5- to 16-membered heterocycle;
provided that R¹ and R², and R² and R³, do not both form Ring A attached to L;
Y′ is N or CR^{Y};
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
[symbol] denotes an optional covalent bond between Y and U;
when the bond between Y and U is absent:
r is 0 or 1;
Y is N or CR^{Y};
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R;
U is hydrogen or C₁₋₆ alkyl optionally substituted with one or more R^{u};
when the bond between Y and U is present:
r is 1;
Y is C;
U is -CH₂-, —C(═O)—, —(C═O)-N(R^{U})-*, or —N═C(R^{U})-*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl; and q is an integer from 0 to 2,
T is of Formula I-2:

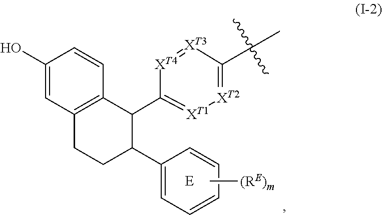

wherein:
each of X^{T1}, X^{T2}, X^{T3}, and X^{T4} is independently N or CRT;
each occurrence of R^{T} is independently hydrogen, halogen, —CN, —NO₂, —OH, —NH₂, C₁₋₆ alkyl, C₁₋₆-alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{E} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 5,
L is of Formula I-3:

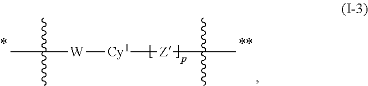

wherein:
* denotes attachment to T and ** denotes attachment to C;
W is absent; or
W is C₁₋₃ alkylene, —O—, —NR^{W}—, or —(C═O)—, wherein the alkylene is optionally substituted by one or more R^{u};
Cy¹ is absent; or
Cy¹ is 6-membered heteroarylene, C₆ arylene, C₃₋₁₂ carbocyclylene, or 3- to 12-membered heterocyclylene, wherein the arylene, heteroarylene, carbocyclylene, or heterocyclylene is optionally substituted by one or more R^{u};
Z′ is absent; or
each Z′ is independently C₁₋₃ alkylene, —O—, —NR^{W}—, —(C═O)—, C₃₋₁₂ carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted with one or more R^{u}; and
p is an integer selected from 0 to 8,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R²; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain aspects, the present disclosure provides compounds of Formula I:

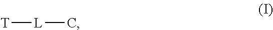

and pharmaceutically acceptable salts, solvates, or stereoisomers thereof,
wherein:
C is of Formula I-1

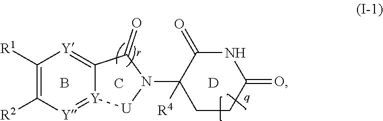

wherein:
[symbol] denotes an optional covalent bond between Y and U;
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R² is *—Cy²—, wherein * denotes attachment to L;
Cy²- is 3- to 12-membered heterocyclylene, wherein the heterocyclylene is optionally substituted with one or more R^{u}; or
R¹ and R², together with the intervening carbon atoms, form Ring A attached to L, wherein Ring A is C₃₋₁₀ carbocycle or 5- to 16-membered heterocycle optionally substituted with one or more Rⁱ;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form Ring A attached to L, wherein Ring A is 5- to 16-membered heterocycle optionally substituted with one or more Rⁱ;
provided that R¹ and R², and R² and R³, do not both form Ring A attached to L;
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O),OR^{b}, OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
Y′ is N or CR^{Y};
R^{Y}′ is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
Y is N or CR^{Y} when the bond between Y and U is absent; or Y is C when the bond between Y and U is present;
R^{Y} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
r is 0 or 1;
U is hydrogen or C₁₋₆ alkyl when the bond between Y and U is absent; or
U is -CH₂-, —C(═O)—, —(C═O)-N(R^{U})-*, or —N═C(R^{U})-* when the bond between Y and U is present;
R^{U} is H or C₁₋₆ alkyl, and * denotes attachment to Ring B;
R⁴ is hydrogen, deuterium, C₁₋₆ haloalkyl, or C₁₋₆ alkyl; and
q is an integer from 0 to 2;
T is of Formula I-2:

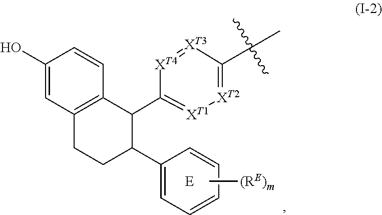

wherein:
each of X^{T1}, X^{T2}, X^{T3}, and X^{T4} is independently N or CRT;
each occurrence of R^{T} is independently hydrogen, halogen, —CN, —NO₂, —OH, —NH₂, C₁₋₆ alkyl, C₁₋₆-alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{E} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
m is an integer selected from 0 to 5,
L is of Formula I-3:

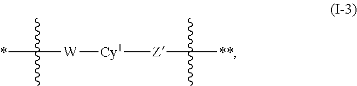

wherein:
W is absent; or
W is -CH₂-, —O—, —NR^{W}—, or —(C═O)—;
R^{W} is hydrogen or C₁₋₆ alkyl;
* denotes attachment to T and ** denotes attachment to C;
Cy¹ is 6-membered heteroarylene, C₆ arylene, C₃₋₁₂ carbocyclylene, or 3- to 12-membered heterocyclylene, wherein the arylene, heteroarylene, carbocyclylene, or heterocyclylene is optionally substituted by one or more R^{u};
Z′ is absent; or
Z′ is —(C(═O))ₚ-(O)ₚ*-(C₁₋₆ alkylene)u-(3- to 6-membered heterocyclylene)ᵥ—(C(═O) )ₚ—(C₁₋₆ alkylene)-(3- to 6-membered heterocyclylene)ᵥ—(C(═O)) p, wherein the alkylene or heterocyclylene is optionally substituted by one or more R^{u};
each occurrence of p, p′, and u is independently 0 or 1; and
each v is an integer independently selected from 0 to 3,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₀ carbocyclyl, 3- to 10-membered heterocyclyl, —SR^{b}, —S(═O)R^{a}, —S(═O)₂R², —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₀ carbocyclyl or 3- to 10-membered heterocyclyl;
each R^{a} is independently C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl; or
R^{c} and R^{d}, together with the nitrogen atom to which they are attached, form 3- to 10-membered heterocyclyl,
wherein each of R^{a}, R^{b}, R^{c}, and R^{d} is independently and optionally substituted with one or more R²; and
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl.

In certain embodiments, when the bond between Y and U is present, U is -CH₂-or-C(═O)—, and r is 1, then either R¹ and R², or R² and R³, together with the intervening carbon atoms, form Ring A attached to L.

In certain embodiments, when the bond between Y and U is present, U is -CH₂-or-C(═O)—, and r is 1, then Ring A is not

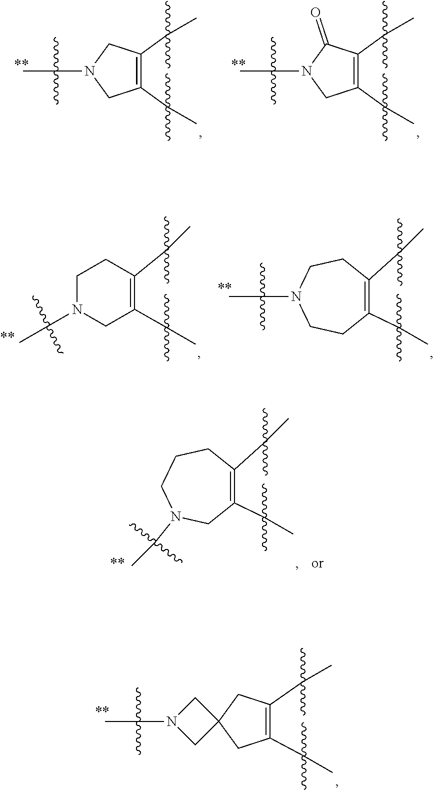 [SMILES: *C1=C(*)CN(*)C1 ; *C1=C(*)C(=O)N(*)C1 ; *C1=C(*)CN(*)CC1 ; *C1=C(*)CCN(*)CC1 ; *C1=C(*)CN(*)CCC1 ; *C1=C(*)CC2(C1)CN(*)C2]

wherein ** denotes attachment to L.

In certain embodiments, when the bond between Y and U is present, U is -CH₂-or-C(═O)—, and r is 1, then Ring A is not

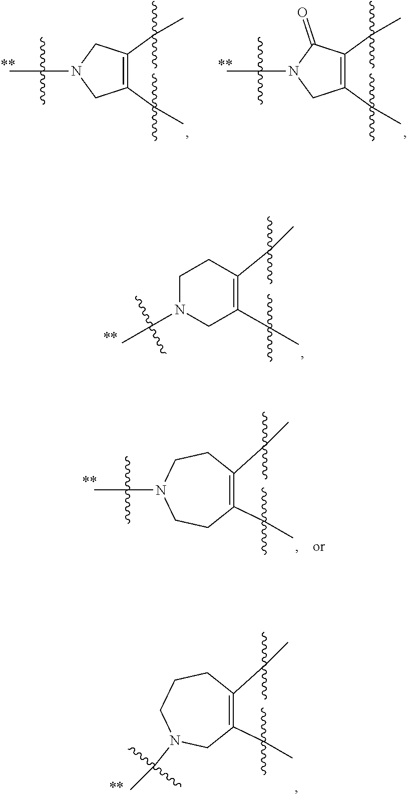 [SMILES: *C1=C(*)CN(*)C1 ; *C1=C(*)C(=O)N(*)C1 ; *C1=C(*)CN(*)CC1 ; *C1=C(*)CCN(*)CC1 ; *C1=C(*)CN(*)CCC1]

wherein ** denotes attachment to L.

In certain embodiments, the compound is not

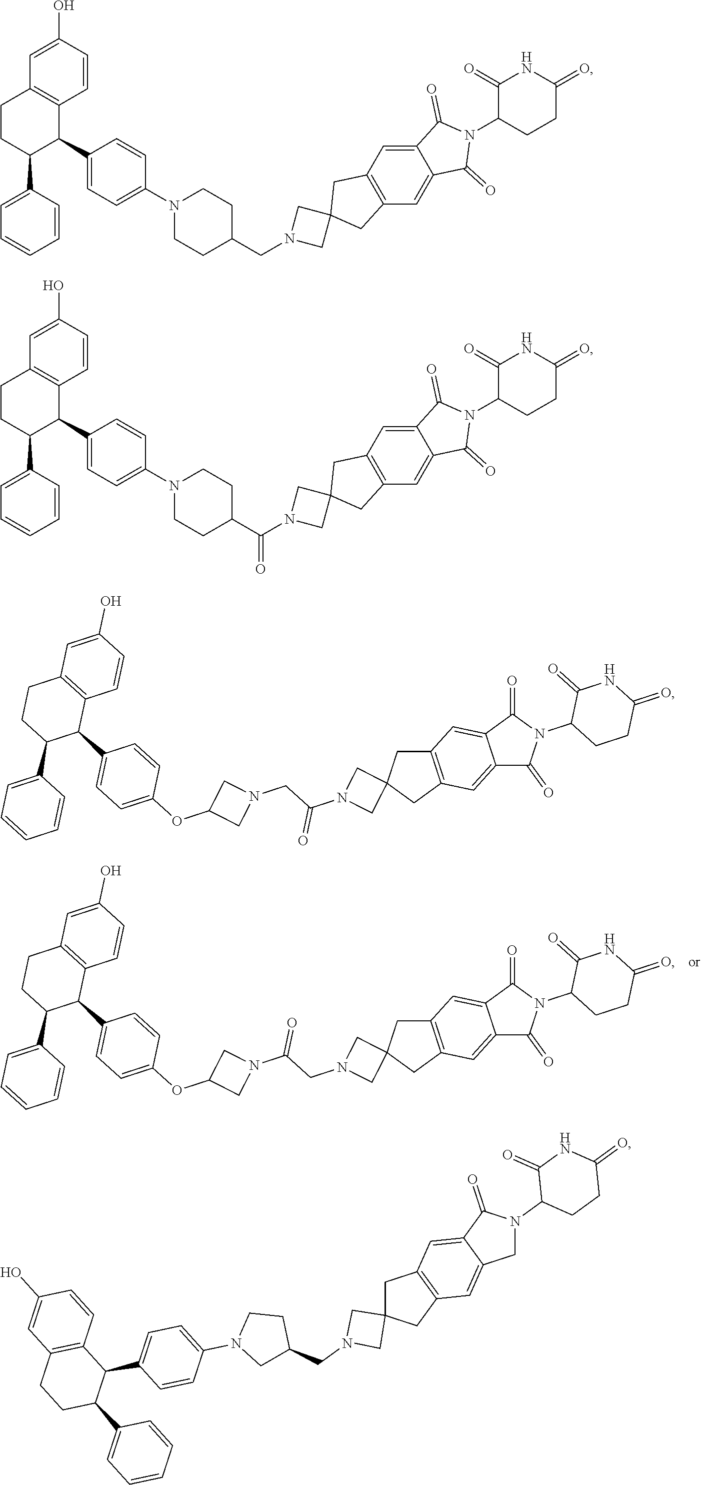

or a pharmaceutically acceptable salt or stereoisomer thereof.

In certain embodiments, the compound is not

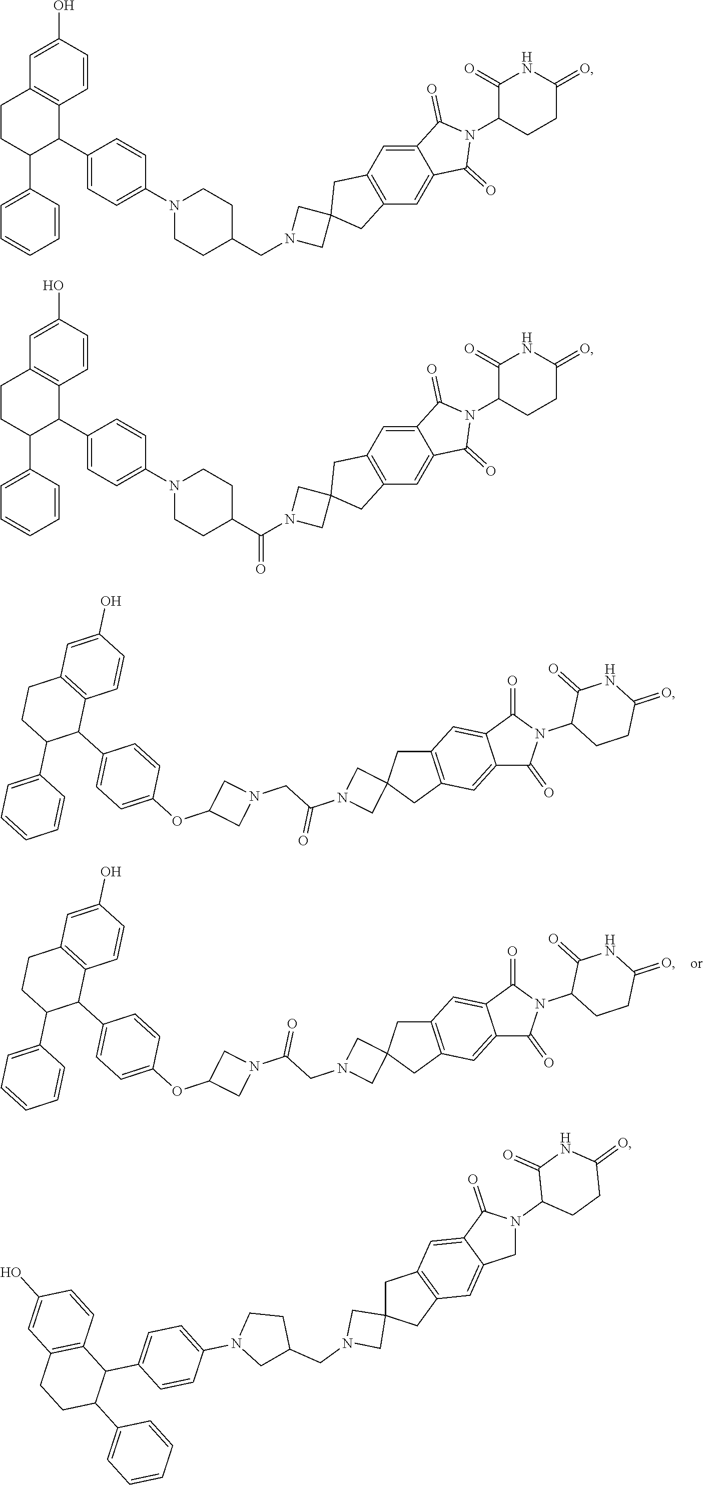

or a pharmaceutically acceptable salt thereof.

In certain embodiments,
1) when the bond between Y and U is present, U is -CH₂-or-C(═O)—, and r is 1, then i) either R¹ and R², or R² and R³, together with the intervening carbon atoms, form Ring A attached to L; and/or ii) Ring A is not

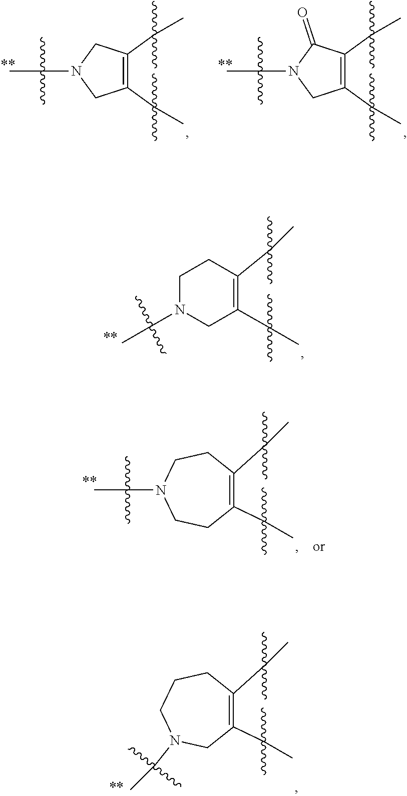 [SMILES: *C1=C(*)CN(*)C1 ; *C1=C(*)C(=O)N(*)C1 ; *C1=C(*)CN(*)CC1 ; *C1=C(*)CCN(*)CC1 ; *C1=C(*)CN(*)CCC1]

wherein ** denotes attachment to L, and/or
2) the compound is not

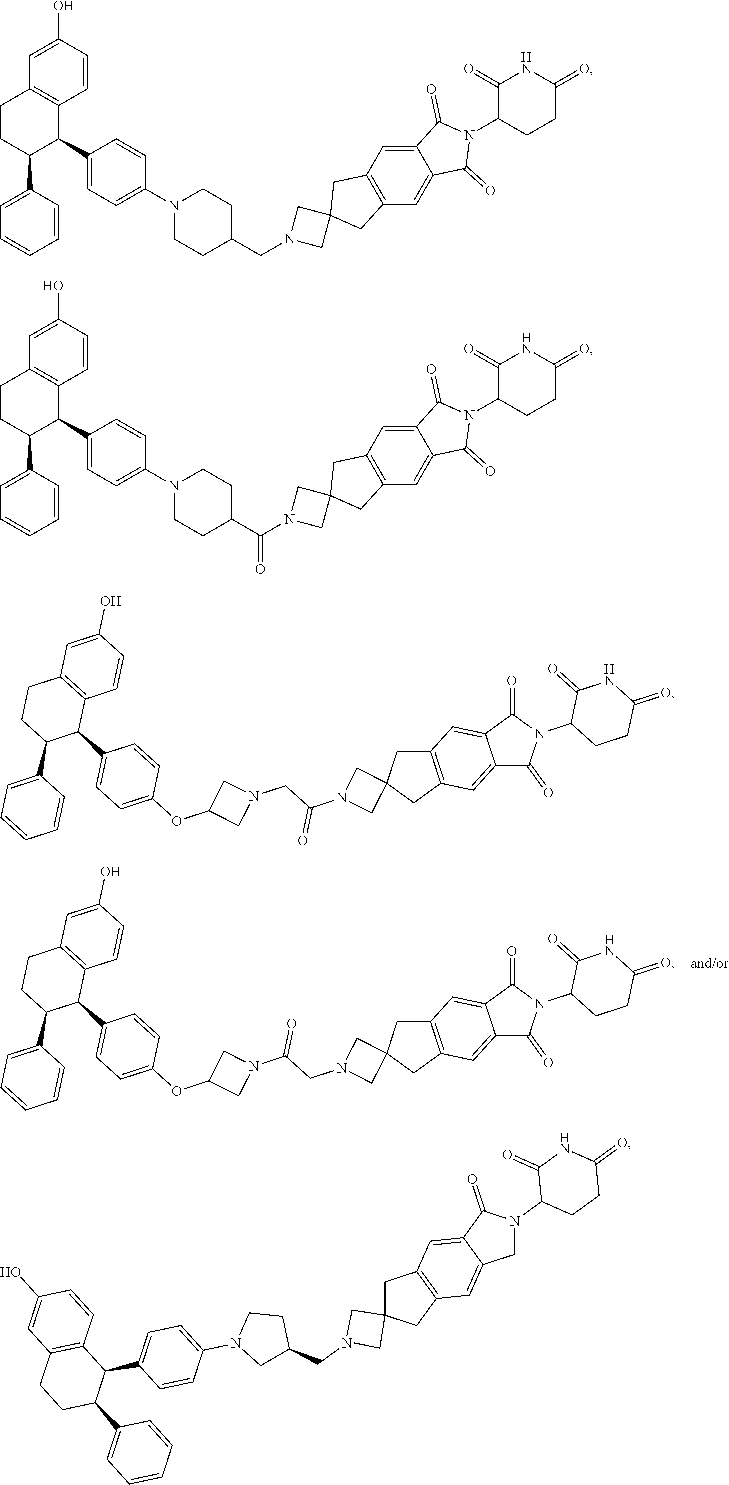

or a pharmaceutically acceptable salt or stereoisomer thereof.

In certain embodiments,
1) when the bond between Y and U is present, U is -CH₂-or-C(═O)—, and r is 1, then i) either R¹ and R², or R² and R³, together with the intervening carbon atoms, form Ring A attached to L; and/or ii) Ring A is not

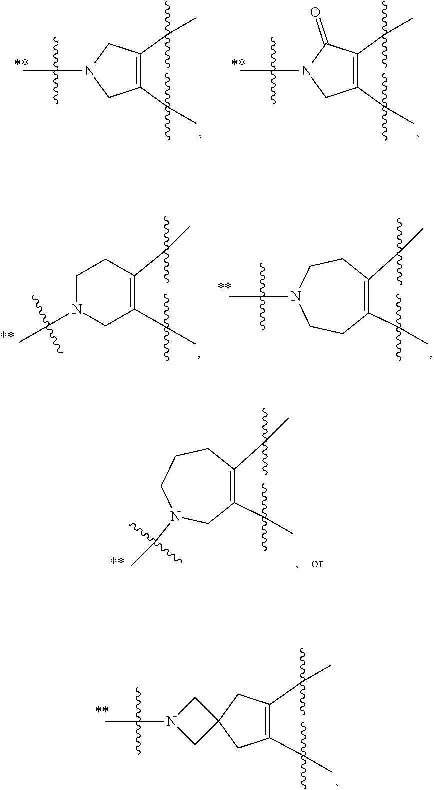 [SMILES: *C1=C(*)CN(*)C1 ; *C1=C(*)C(=O)N(*)C1 ; *C1=C(*)CN(*)CC1 ; *C1=C(*)CCN(*)CC1 ; *C1=C(*)CN(*)CCC1 ; *C1=C(*)CC2(C1)CN(*)C2]

wherein ** denotes attachment to L, and/or
2) the compound is not

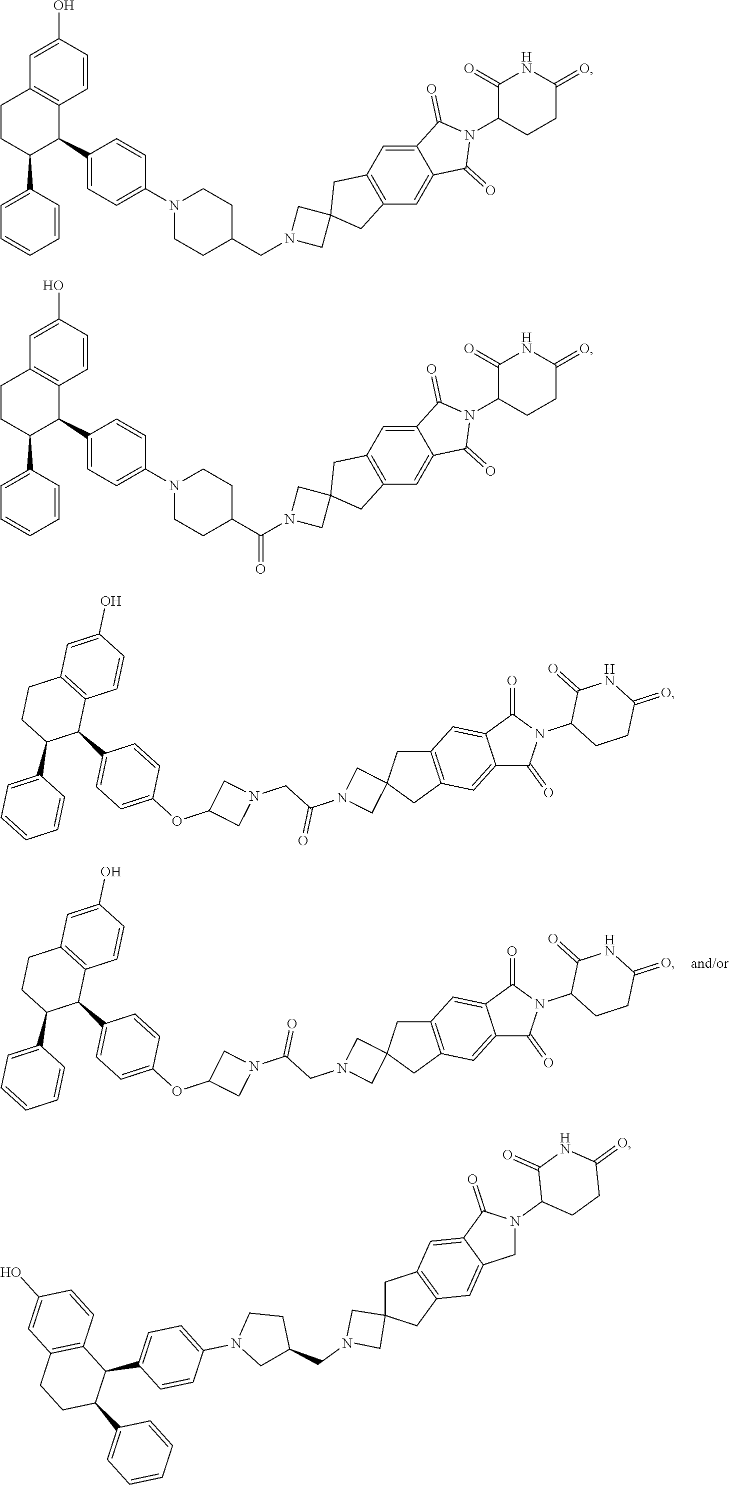

or a pharmaceutically acceptable salt or stereoisomer thereof.

In certain embodiments,
1) when the bond between Y and U is present, U is -CH₂-or-C(═O)—, and r is 1, then i) either R¹ and R², or R² and R³, together with the intervening carbon atoms, form Ring A attached to L; and/or ii) Ring A is not

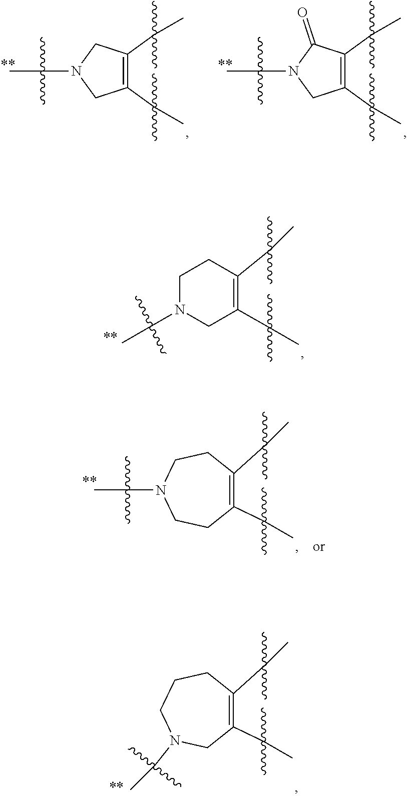 [SMILES: *C1=C(*)CN(*)C1 ; *C1=C(*)C(=O)N(*)C1 ; *C1=C(*)CN(*)CC1 ; *C1=C(*)CCN(*)CC1 ; *C1=C(*)CN(*)CCC1]

wherein ** denotes attachment to L, and/or
2) the compound is not

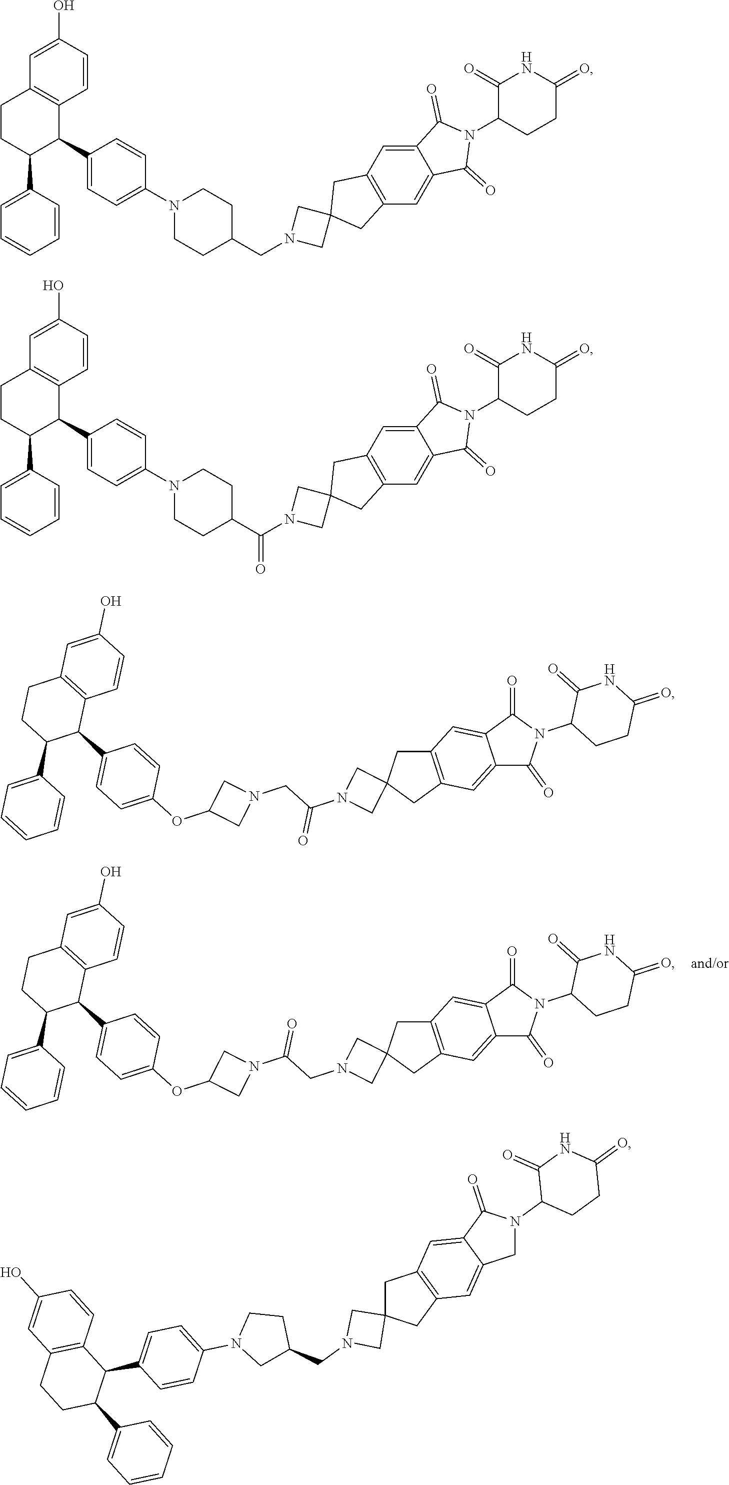

or a pharmaceutically acceptable salt or stereoisomer thereof.

In certain embodiments,
1) when the bond between Y and U is present, U is -CH₂-or-C(═O)—, and r is 1, then i) either R¹ and R², or R² and R³, together with the intervening carbon atoms, form Ring A attached to L; and/or ii) Ring A is not

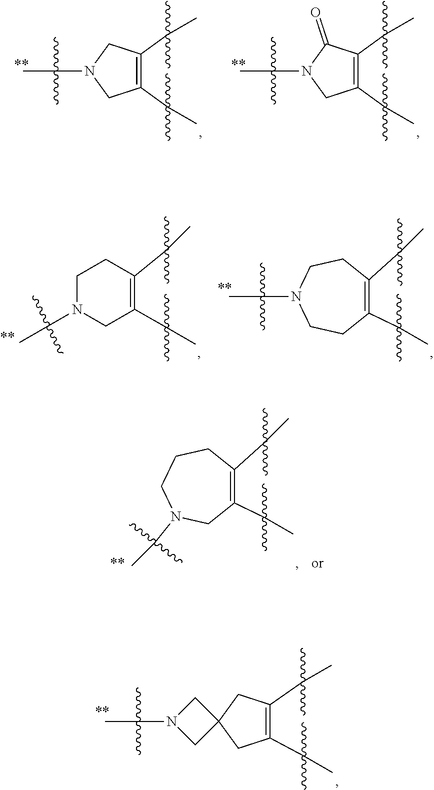 [SMILES: *C1=C(*)CN(*)C1 ; *C1=C(*)C(=O)N(*)C1 ; *C1=C(*)CN(*)CC1 ; *C1=C(*)CCN(*)CC1 ; *C1=C(*)CN(*)CCC1 ; *C1=C(*)CC2(C1)CN(*)C2]

wherein ** denotes attachment to L, and/or
2) the compound is not

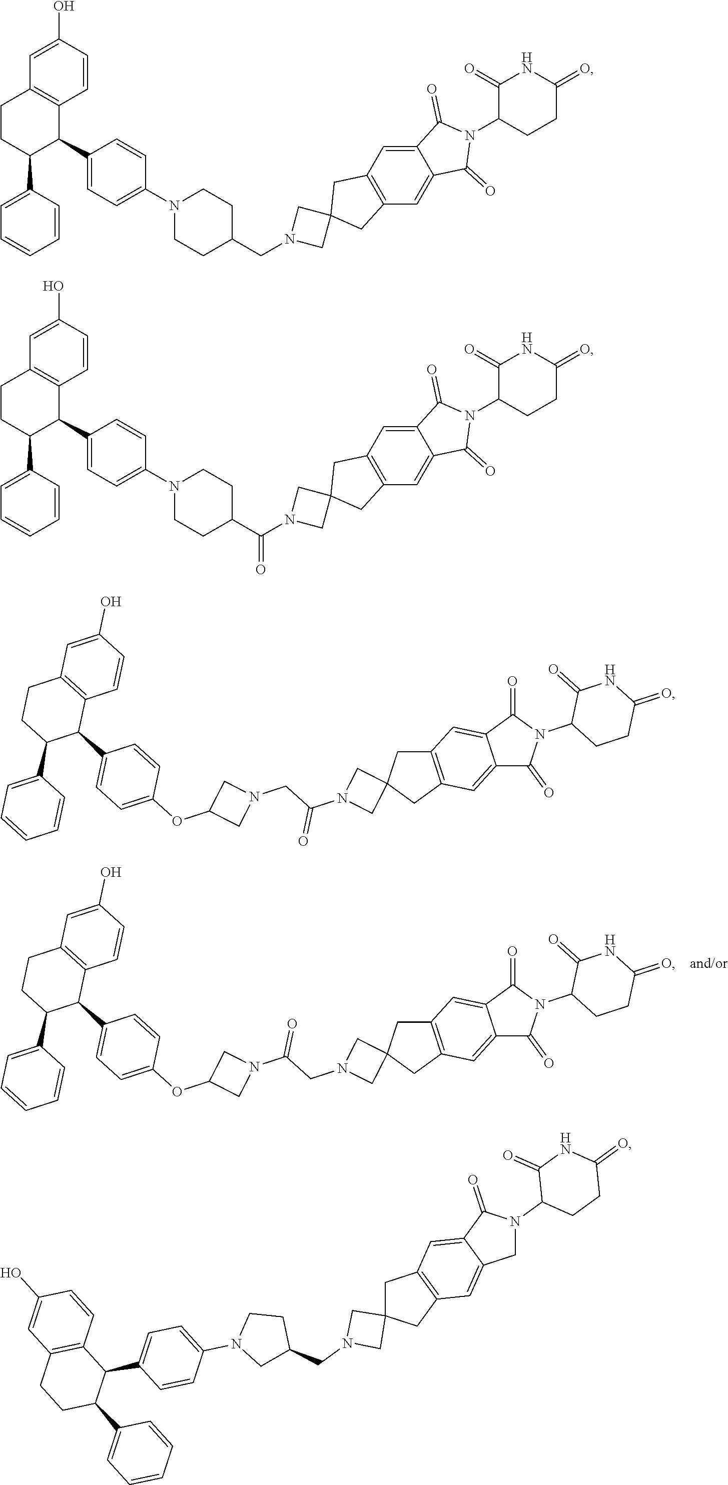

or a pharmaceutically acceptable salt or stereoisomer thereof.

In certain embodiments,
1) when the bond between Y and U is present, U is -CH₂-or-C(═O)—, and r is 1, then i) either R¹ and R², or R² and R³, together with the intervening carbon atoms, form Ring A attached to L; and/or ii) Ring A is not

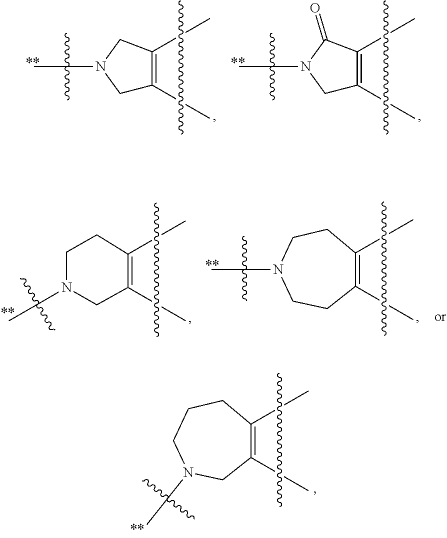

wherein ** denotes attachment to L, and/or
2) the compound is not

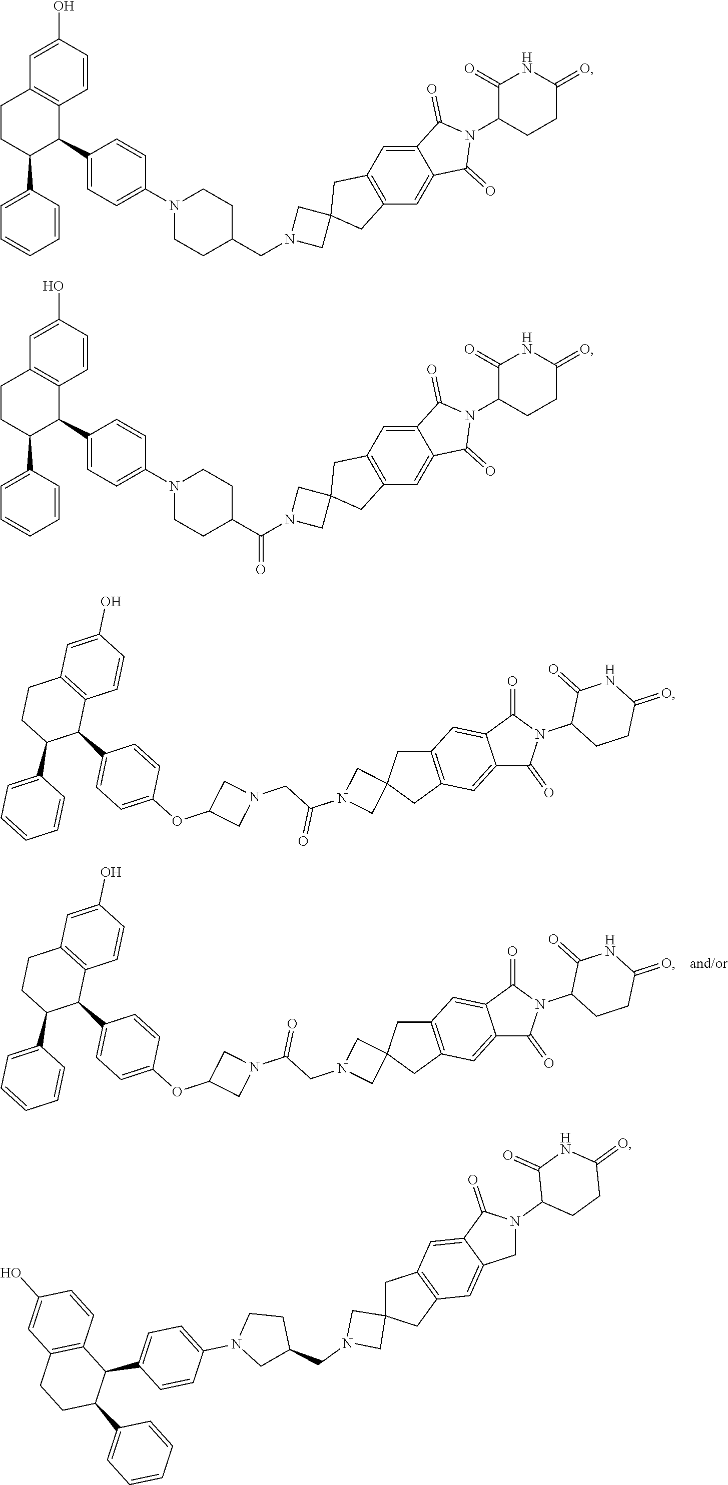

or a pharmaceutically acceptable salt or stereoisomer thereof.

In certain embodiments,
1) when the bond between Y and U is present, U is -CH₂-or-C(═O)—, and r is 1, then i) either R¹ and R², or R² and R³, together with the intervening carbon atoms, form Ring A attached to L; and/or ii) Ring A is not

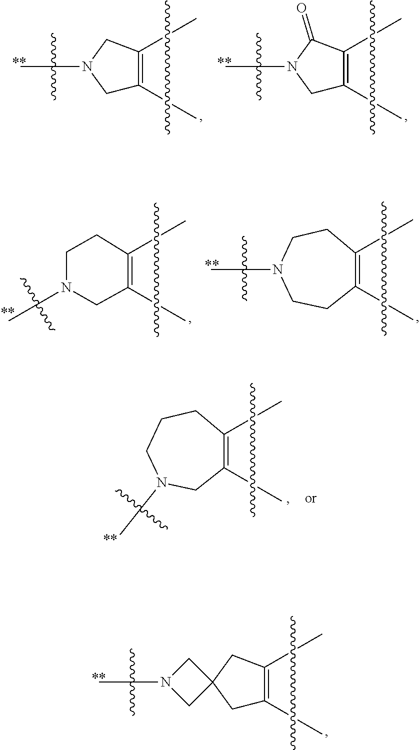

wherein ** denotes attachment to L, and/or
2) the compound is not

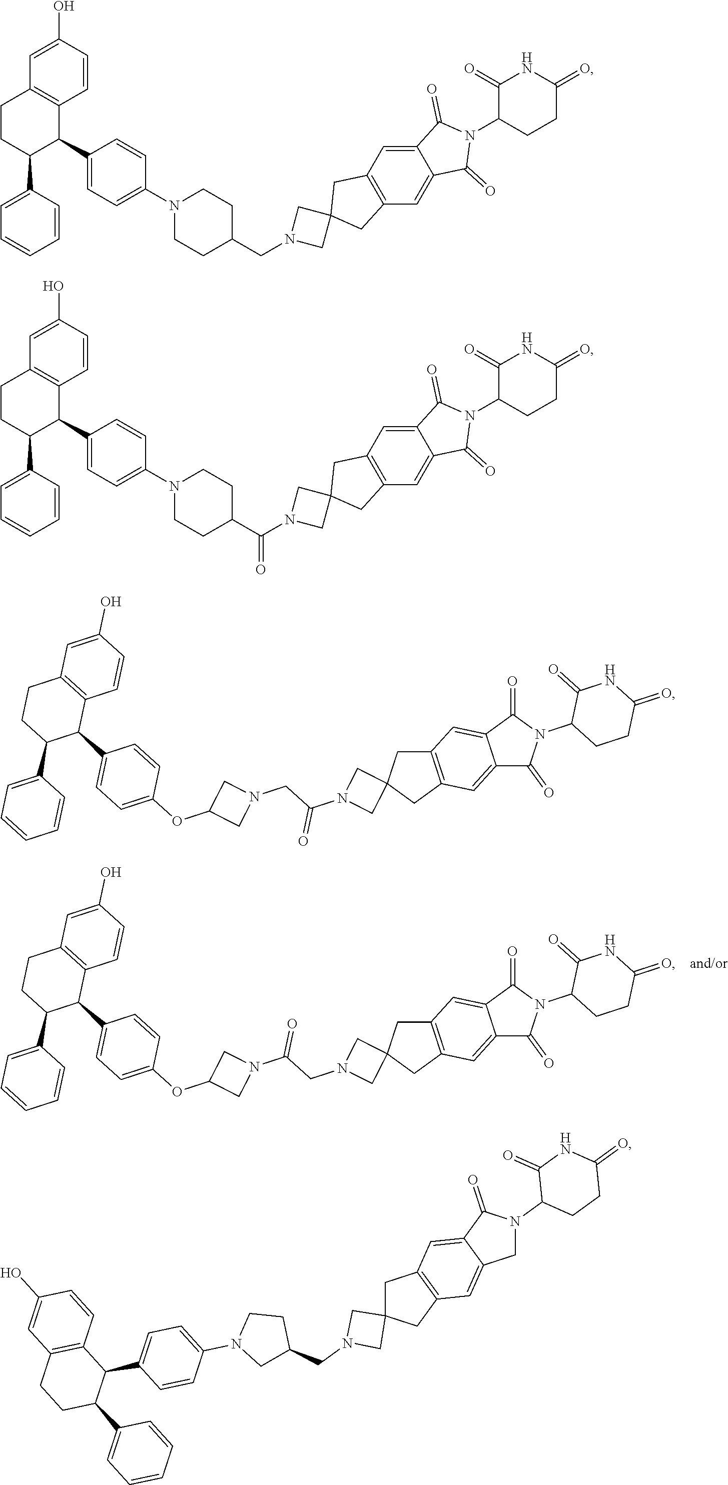

or a pharmaceutically acceptable salt or stereoisomer thereof.

In certain embodiments,
1) when the bond between Y and U is present, U is -CH₂-or-C(═O)—, and r is 1, then i) either R¹ and R², or R² and R³, together with the intervening carbon atoms, form Ring A attached to L; and/or ii) Ring A is not

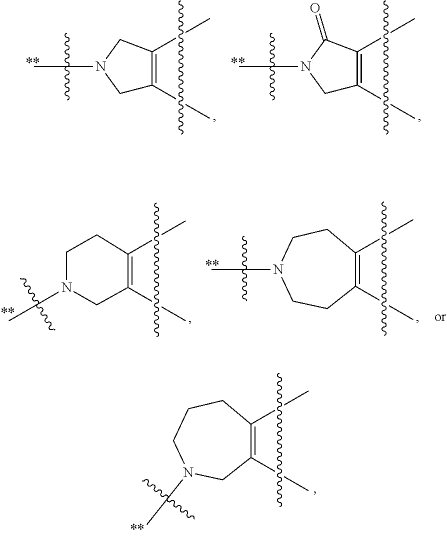

wherein ** denotes attachment to L, and/or
2) the compound is not

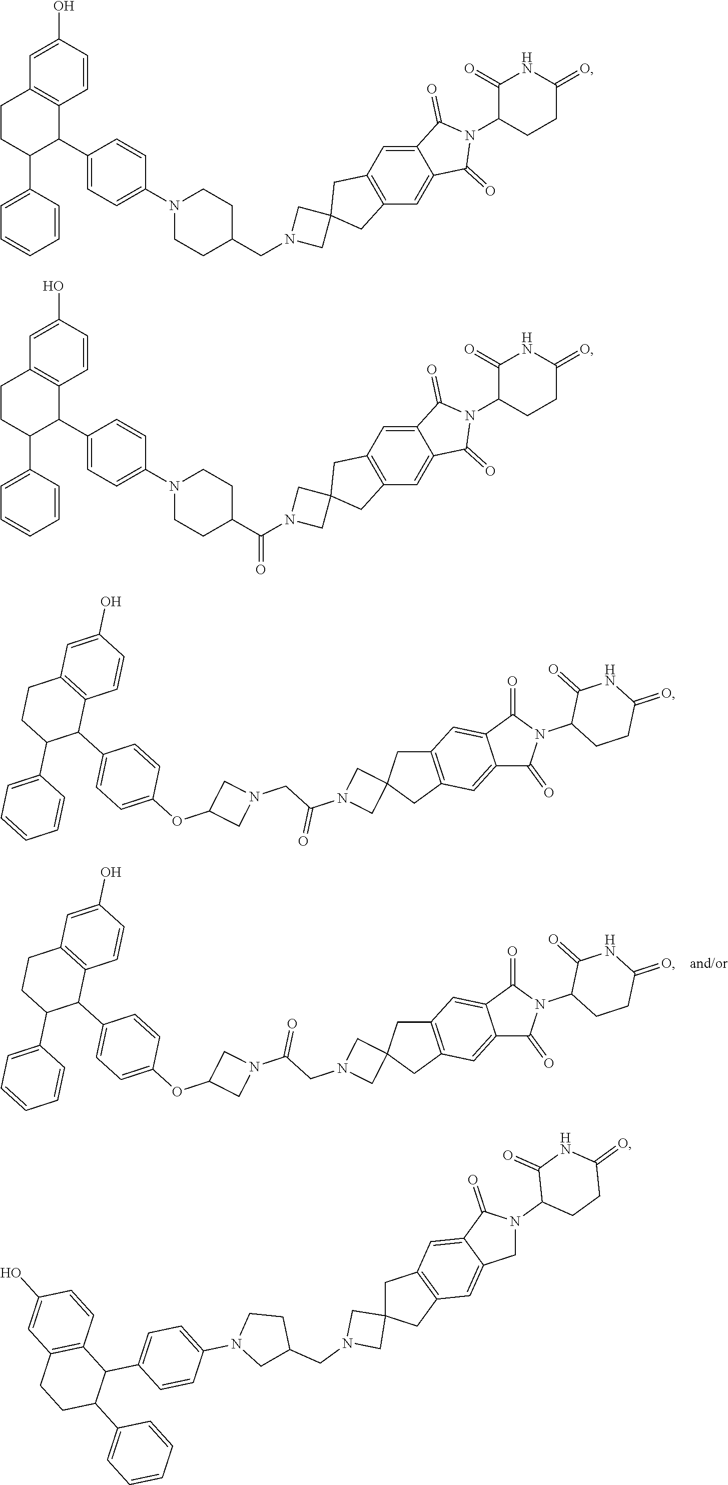

or a pharmaceutically acceptable salt or stereoisomer thereof.

In certain embodiments,
1) when the bond between Y and U is present, U is -CH₂-or-C(═O)—, and r is 1, then i) either R¹ and R², or R² and R³, together with the intervening carbon atoms, form Ring A attached to L; and/or ii) Ring A is not

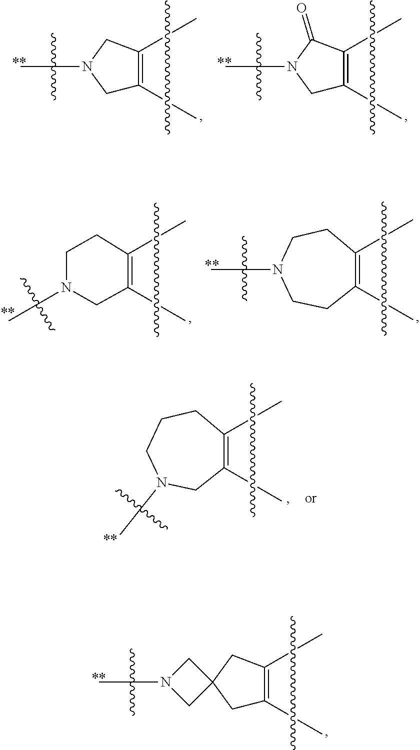

wherein ** denotes attachment to L, and/or
2) the compound is not

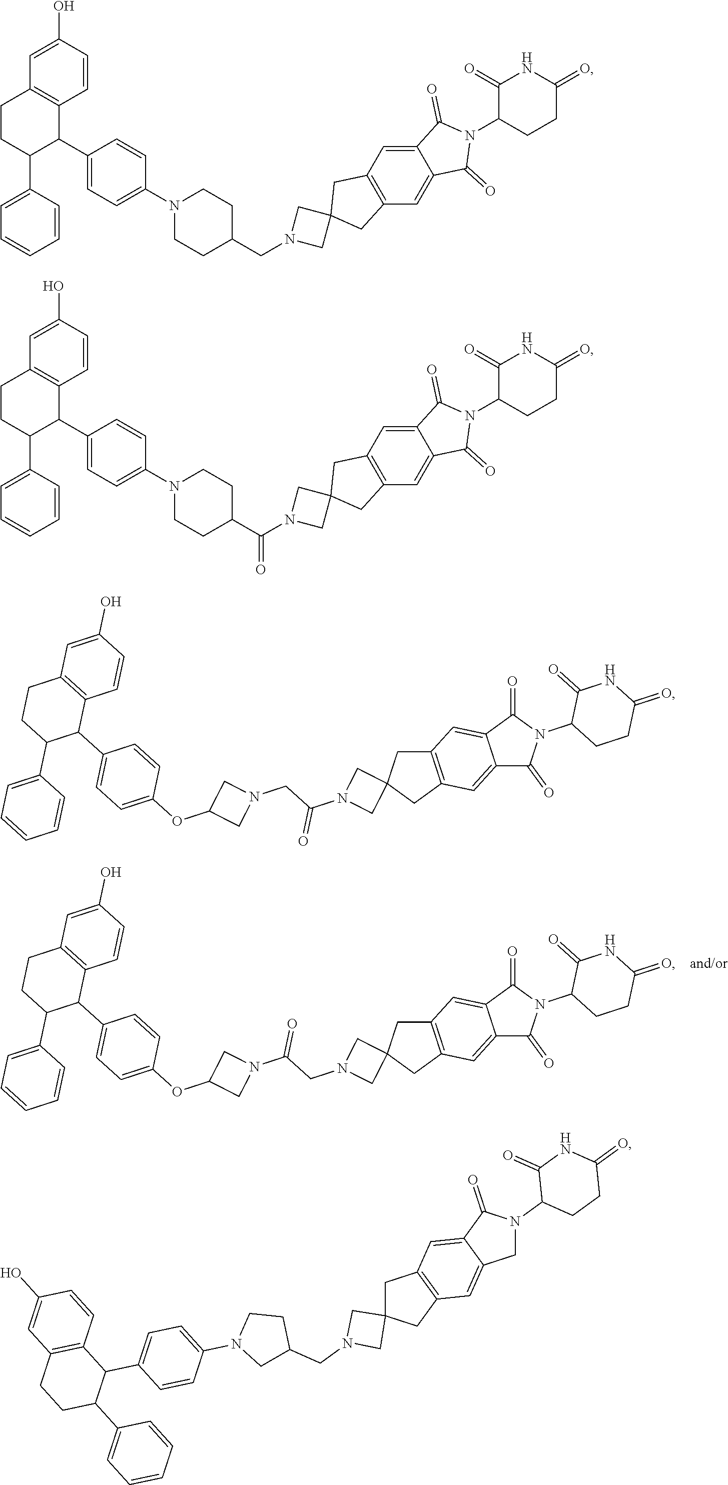

or a pharmaceutically acceptable salt or stereoisomer thereof.

In certain embodiments, C is of Formula 1-1-1

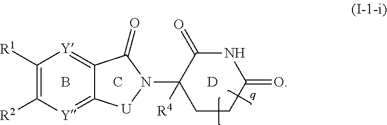

In certain embodiments, C is of Formula 1-1

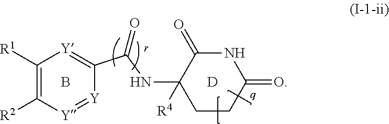

In certain embodiments, C is of Formula I′-1-i

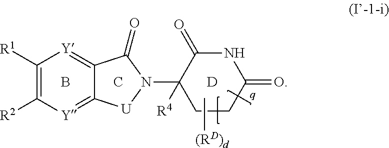

In certain embodiments, C is of Formula I′-1-fi

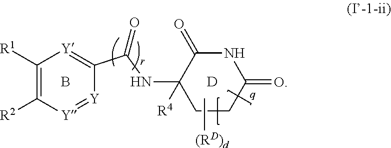

In certain embodiments, U is -CH₂-or-C(═O)—. In certain embodiments, U is -CH₂-or-C(═O)-when the bond between Y and U is present. In certain embodiments, U is —(C═O)-N(R^{U})-* or —N═C(R^{U})-* when the bond between Y and U is present.

In certain embodiments, R¹ is hydrogen, halogen (e.g., -F, -Cl, -Br, or -I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), 1-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, 1-butylpentylamino, n-butylhexylamino, i butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments. R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R¹ is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments. R¹ is hydrogen, halogen, or C₁₋₆ alkoxy.

In certain embodiments. R² is *—Cy²—, wherein * denotes attachment to L.

In certain embodiments, —Cy²- is C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptyl (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₅), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5] decenylene (C₁₀)) or 3- to 12-membered heterocyclylene (e.g., heterocyclylene comprising one or two 3- to 8-membered rings and 1-5 heteroatoms selected from N, O, and S), wherein the carbocyclylene or heterocyclylene is optionally substituted with one or more R^{u}.

In certain embodiments, *—Cy²- is C₅₋₁₂ fused carbocyclene or 5- to 12-membered fused heterocyclylene, wherein the carbocyclene or heterocyclylene is optionally substituted with one or more R^{u}.

In certain embodiments. *—Cy²- is 5- to 12-membered fused heterocyclylene comprising 1 or 2 nitrogen atoms, wherein the heterocyclene is optionally substituted with one or more R^{u}.

In certain embodiments. *—Cy²- is

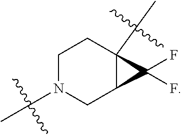 [SMILES: *N1CC[C@]2(*)[C@H](C1)C2(F)F]

In certain embodiments, R¹ and R², together with the intervening carbon atoms, form Ring A attached to L, wherein Ring A is optionally substituted C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) or 5- to 16-membered heterocyclyl (e.g., heterocyclyl comprising one or two 5- to 8-membered rings and 1-5 heteroatoms selected from N, O, and S).

In certain embodiments. Y″ is N or CR³.

In certain embodiments, R³ is hydrogen, halogen (e.g., -F, -Cl, -Br, or -I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamine, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{b}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, -OC(═O)R^{a}, —OC(═O)OR^{b}, —OC(═O)NR^{c}R^{d}, —C(═O)R^{b}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments. R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R³ is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments. R³ is hydrogen, halogen, or C₁₋₆ alkoxy.

In certain embodiments, R² and R³, together with the intervening carbon atoms, form Ring A attached to L, wherein Ring A is optionally substituted C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) or 5- to 16-membered heterocyclyl (e.g., heterocyclyl comprising one or two 3- to 8-membered rings and 1-5 heteroatoms selected from N, O, and S).

In certain embodiments. R¹ and R², and R² and R³, do not both form Ring A attached to L.

In certain embodiments. Y′ is N or CR^{Y}.

In certain embodiments, R^{Y} is hydrogen, halogen (e.g., -F, -Cl, -Br, or -I), —CN, —NO₂, —OH, -NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (CI), ethoxy (C₂), propoxy (C₃), i-propoxy (C₃), n-butoxy (C₄), i-butoxy (C₄), s-butoxy (C₄), 1-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-/-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments. R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments. R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments. R^{Y} is hydrogen, halogen, —CN, —NO₂, —OH, —NH₂. C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments. R^{Y} is hydrogen, halogen, or C₁₋₆ alkoxy.

In certain embodiments, i) when the bond between Y and U is absent, then r is 0 or 1, Y is N or CR^{Y}, and U is hydrogen or C₁₋₆ alkyl optionally substituted with one or more R^{u}.

In certain embodiments. Y is N. In certain embodiments, Y is CR^{Y}.

In certain embodiments, R^{Y} is hydrogen, halogen (e.g., -F, -Cl, -Br, or -I), —CN, —NO₂, —OH, -NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), 1-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{Y} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments. R^{Y} is hydrogen, halogen, or C₁₋₆ alkoxy.

In certain embodiments, ii) when the bond between Y and U is present, then r is 1, Y is C, and U is -CH₂-, —C(═O)—, —(C═O)-N(R^{U})-*, or —N═C(R^{U}).*.

In certain embodiments, R^{U} is H or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}, and * denotes attachment to Ring B.

In certain embodiments, R⁴ is hydrogen, deuterium, C₁₋₆ haloalkyl (e.g., C₁₋₆ alkyl comprising 1-6 halogen atoms selected from F, Cl, Br, and I), or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)).

In certain embodiments, each R^{D} is independently oxo, halogen (e.g., -F, -Cl, -Br, or -I), —CN, -NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamine, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{D} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, d is an integer from 0 to 4. In certain embodiments, d is 0. In certain embodiments, d is 1. In certain embodiments, d is 2. In certain embodiments, d is 3. In certain embodiments, d is 4.

In certain embodiments, q is an integer from 0 to 2. In certain embodiments, q is 0. In certain embodiments, q is 1. In certain embodiments, q is 2.

In certain embodiments, Ring A is optionally substituted 7- to 16-membered fused heterocycle.

In certain embodiments, Ring A is

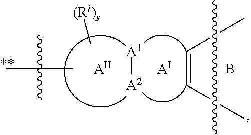

wherein:
** denotes attachment to L;
Ring A^{I} and Ring A^{II} are independently C₄₋₈ carbocycle or 4- to 8-membered heterocycle; wherein at least one of Ring A^{III} and Ring A^{IV} is 4- to 8-membered heterocycle;
A¹ and A² are independently C, CR^{Ax}, or N;
R^{Ax} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits,
wherein each Rⁱ may independently be present on cither Ring A^{I} or Ring A^{II}.

In certain embodiments, Ring A is

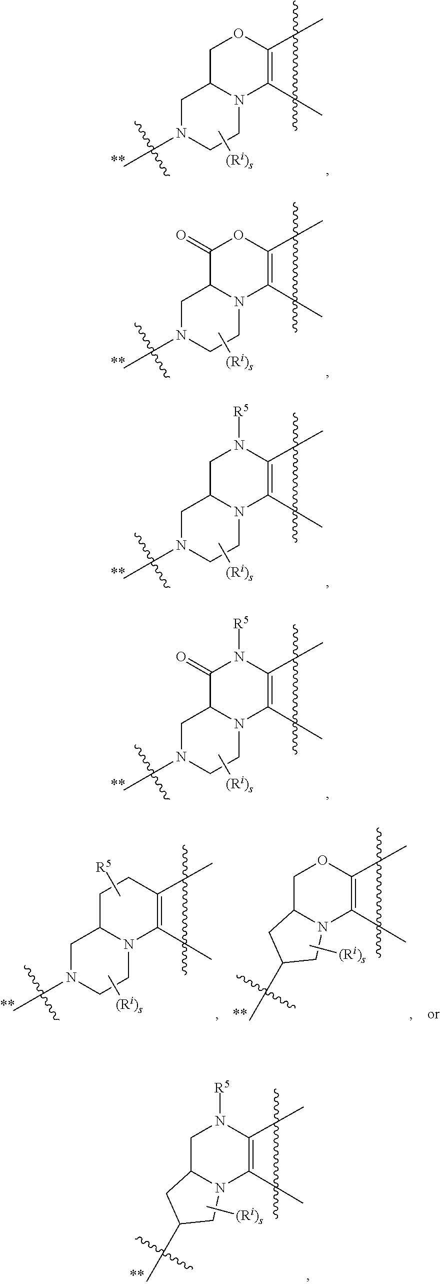

wherein:
* denotes attachment to L;
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{a}, —NR^{c}S(═O)₂R^{a}, —NR^{c}S(═O)R^{a}, —NR^{c}S(═O)₂OR°, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, Ring A is

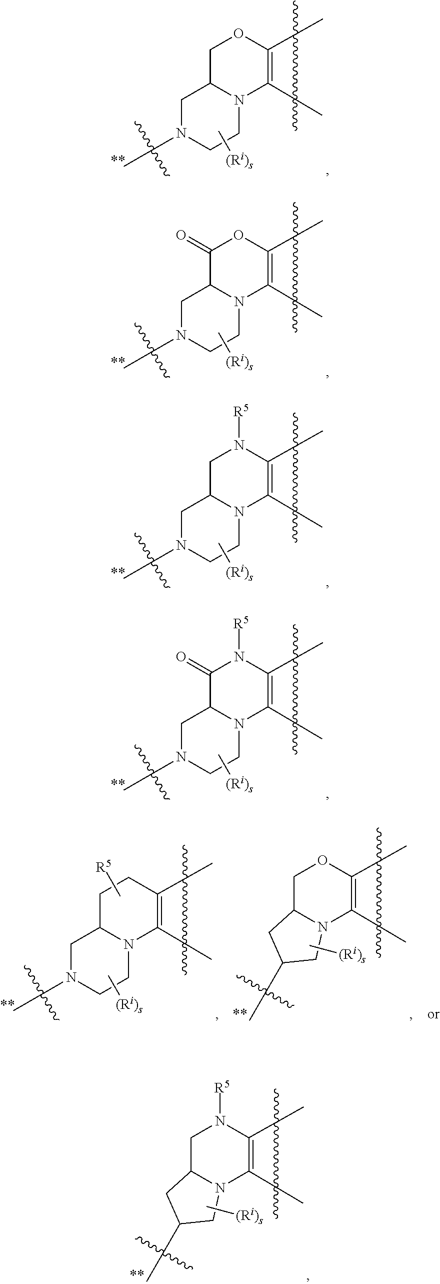

wherein:
** denotes attachment to L;
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, Ring A is

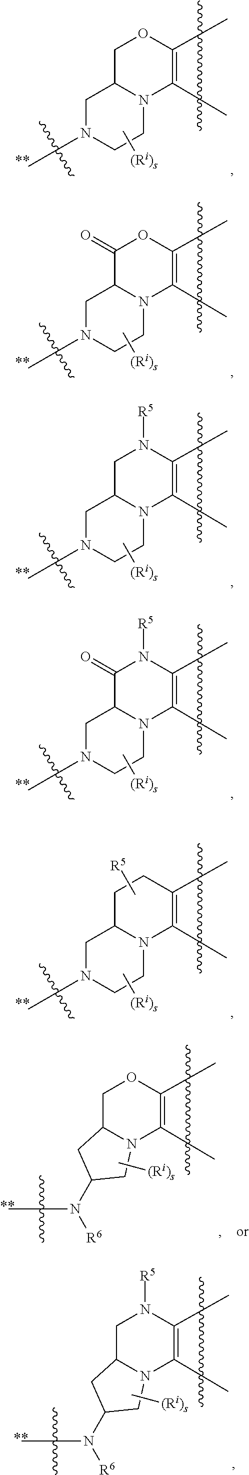

wherein:
** denotes attachment to L;
R⁵ is hydrogen or C₁₋₆ alkyl optionally substituted with one or more R^{u};
each R⁶ is independently hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and s is an integer selected from 0 to 8, as valency permits.

In certain embodiments, Ring A^{I} and Ring A^{II} are independently C₄₋₈ carbocycle (e.g., cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), or cyclooctenyl (C₈)) or 4- to 8-membered heterocycle (e.g., heterocyclyl comprising one or two 4- to 8-membered rings and 1-4 heteroatoms selected from N, O, and S); wherein at least one of Ring A^{III} and Ring A^{IV} is 4- to 8-membered heterocycle.

In certain embodiments. A¹ and A² are independently C, CRAN, or N.

In certain embodiments, R^{Ax} is hydrogen, halogen (e.g., -F, -Cl, -Br, or -I), —CN, —NO₂, —OH, -NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments. R^{Ax} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments. R^{Ax} is hydrogen, halogen, —CN, —NO₂, —OH, —NH₂. C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments. R^{Ax} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R⁶ is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R⁶ is hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R⁶ is hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R⁶ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, Ring A is optionally substituted with one or more R^{u}.

In certain embodiments, R^{u} is R^{Ax}. In certain embodiments, R^{u} is R⁵. In certain embodiments, R^{u} is Rⁱ.

In certain embodiments, Ring A is optionally substituted 7- to 16-membered spiro heterocycle.

In certain embodiments, Ring A is:

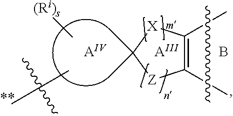

wherein:
** denotes attachment to L;
Ring A² is C₃₋₈ carbocycle or 3- to 8-membered heterocycle;
each X is independently —C(R^{X1})₂-, —NR^{X2}-, —O—, —S—, —S(═O)—, or —S(═O)₂-;
each Z is independently —C(R^{Z1})₂-, —NR^{Z2}-, —O—, —S—, —S(═O)-, or —S(═O)₂-:
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(-O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R^{Z2} is independently hydrogen or C₁₋₆ alkyl optionally substituted with one or more R^{u};
m′ and n′ are independently an integer selected from 0-3, wherein m′ and n′ are not both 0;
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R², —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, Ring A is:

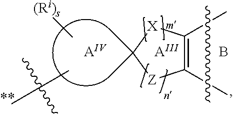

wherein:
** denotes attachment to L;
Ring A^{IV} is C₃₋₈ carbocycle or 3- to 8-membered heterocycle;
each X is independently —C(R^{X1})₂-, —NR^{X2}, —O—, —S—, —S(═O)—, or —S(═O)₂-;
each Z is independently —C(R^{Z1})₂-, —NR^{Z2}-, —O—, —S—, —S(═O)—, or —S(═O)₂-;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R^{Z2} is independently hydrogen or C₁₋₆ alkyl optionally substituted with one or more R^{u};
m′ and n′ are independently an integer selected from 0-3, wherein m′ and n′ are not both 0;
s is an integer selected from 0 to 8, as valency permits, and
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
provided that when none of m′ and n′ is 0, then Ring A^{I} is 4- to 9-membered heterocycle. In certain embodiments, Ring A is:

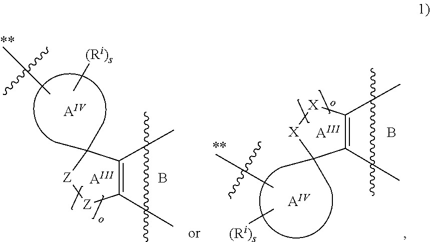

wherein o is 0 or 1; or

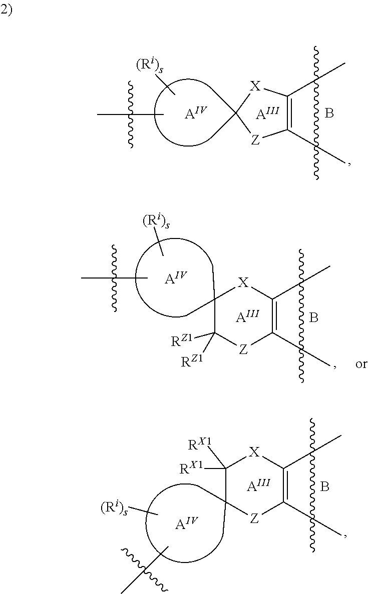

wherein ** denotes attachment to L.

In certain embodiments, Ring A^{IV} is C₃₋₈ carbocycle (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), or bicyclo[2.2.2]octanyl (C₈)) or 3- to 8-membered heterocycle (e.g., heterocyclyl comprising one or two 3- to 8-membered rings and 1-5 heteroatoms selected from N, O, and S).

In certain embodiments, each X is independently —C(R^{X1})₂-, —NR^{X2}-, —O—, —S—, —S(═O)—, or —S(═O)₂—. In certain embodiments, each X is independently —C(R^{X1})₂-, —NR^{X2}—, an —O—.

In certain embodiments, each Z is independently —C(R^{Z1})₂-, —NR^{Z2}-, —O—, —S—, —S(═O)—, or —S(═O)₂—. In certain embodiments, each Z is independently —C(R^{Z1})₂-, —NR^{Z2}-, or —O—.

In certain embodiments, each occurrence of R^{X1} and R^{Z1} is independently hydrogen, halogen (e.g., -F, -Cl, -Br, or -I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)). C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)). C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₁), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, -NR^{c}S(═O₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino. C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino. C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, two geminal R^{X1} or two geminal R^{Z1} together form oxo.

In certain embodiments, two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u}.

In certain embodiments, two geminal R^{X1} or two geminal R^{Z1}, together with the carbon atom to which they are attached, form C₃₋₁₂ carbocyclyl or 3- to 12-membered heterocyclyl, wherein the carbocyclyl or heterocyclyl is optionally substituted with one or more R^{u}.

In certain embodiments, each occurrence of R^{X2} and R^{Z2} is independently hydrogen or C₁₋₆-alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, m′ is an integer selected from 0 to 3. In certain embodiments, m′ is 0. In certain embodiments, m′ is 1. In certain embodiments, m′ is 2. In certain embodiments, m′ is 3.

In certain embodiments, n′ is an integer selected from 0 to 3. In certain embodiments, n′ is 0. In certain embodiments, n′ is 1. In certain embodiments, n′ is 2. In certain embodiments, n′ is 3.

In certain embodiments, m′ and n′ are not both 0.

In certain embodiments. Ring A is optionally substituted with one or more R^{u}.

In certain embodiments, R^{u} is R^{X1}. In certain embodiments, R^{u} is R^{X2}. In certain embodiments, R^{u} is R^{Z1}. In certain embodiments, R^{u} is R²². In certain embodiments, R^{u} is Rⁱ.

In certain embodiments, Ring A is optionally substituted 5- to 6-membered heterocycle.

In certain embodiments, Ring A is

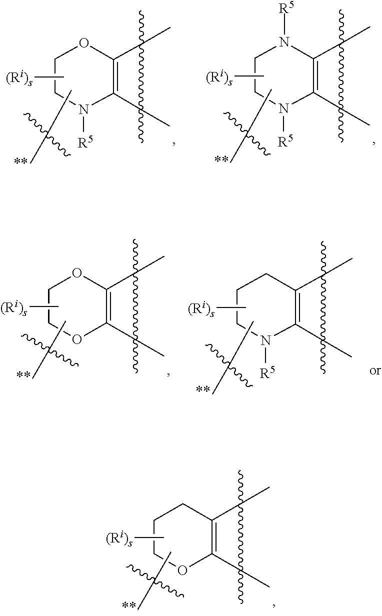

wherein:
** denotes attachment to L;
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, Ring A is optionally substituted with one or more R^{u}.

In certain embodiments. R^{u} is R⁵. In certain embodiments, R^{u} is Rⁱ.

In certain embodiments. R⁵ is hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, each Rⁱ is independently oxo, halogen (e.g., -F, -Cl, -Br, or -I), —CN, -NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), 1-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-1-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)). C₂₋₆ alkynyl (e.g., ethynyl (C₂), 1-propynyl (C₃). 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₁), cycloheptadienyl (C₁), cycloheptatrienyl (C₁), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O))R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more Ru

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8. In certain embodiments, each of X^{T1}, X^{T2}, X^{T3}, and X^{T4} is CRT.

In certain embodiments, each of X^{T1}, X^{T2}, X^{T3}, and X^{T4} is CH. In certain embodiments, each of X^{T1} and X^{T4} is CH, one of X^{T2} and X^{T3} is CH, and the other one of X^{T2} and X^{T3} is CF. In certain embodiments, one of X^{T1} and X^{T4} is CF or C(OCH₃), the other one of XTI and X^{T4} is CH, and each X^{T2} and X^{T3} is CH. In certain embodiments, X^{T1} is C(OCH₃), X^{T3} is CF, and each of X^{T2} and X^{T4} is CH. In certain embodiments, X^{T2} is CF, X^{T4} is C(OCH₃), and each of XTI and X^{T3} is CH. In certain embodiments, X^{T1} is C(OCH₃), X^{T2} is CF, and each of X^{T3} and X^{T4} is CH.

In certain embodiments, one of X^{T1}, X^{T2}, X^{T3}, and X^{T4} is N.

In certain embodiments, one of X^{T1} and X^{T4} is N, the other one of X^{T1} and X^{T4} is CH, and each of X^{T2} and X^{T3} is CH. In certain embodiments, one of X^{T2} and X^{T3} is N, the other one of X^{T2} and X^{T3} is CH, and each of X^{T1} and X^{T4} is CH.

In certain embodiments, two of X^{T1}, X^{T2}, X^{T3}, and X^{T4} are N.

In certain embodiments, each of X^{T1} and X^{T4} is CH, and each of X^{T2} and X^{T3} is N.

In certain embodiments, T is

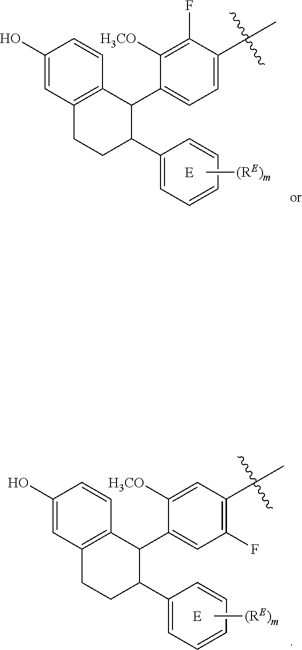

In certain embodiments, each R^{T} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, each R^{T} is independently hydrogen, C₁₋₆ alkoxy, or halogen.

In certain embodiments, each R^{E} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{E} is halogen.

In certain embodiments, m is 0. In certain embodiments, m is 1. In certain embodiments, m is 2. In certain embodiments, m is 3. In certain embodiments, m is 4. In certain embodiments, m is 5.

In certain embodiments. L is of Formula I′-3:

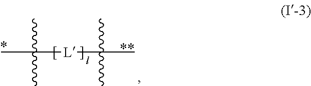

wherein:
* denotes attachment to T, and ** denotes attachment to C;
each L′is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L})—, —C(═O)O—, —N(R^{L})—, —O—, —S—, or —S(═O)₂—, wherein the alkylene, heteroalkylene, alkenylene, alkynylene, carbocyclylene, heterocyclylene, arylene, or heteroarylene is optionally substituted with one or more R^{u};
each occurrence of R^{L} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
1 is an integer selected from 0 to 10,

In certain embodiments, each L′ is independently C₁₋₆ alkylene (e.g., methylene (-CH₂-), ethylene (-CH₂CH₂—), n-propylene (-CH₂CH₂CH₂—), n-butylene (-CH₂CH₂CH₂CH₂—), n-pentylene (-CH₂CH₂CH₂CH₂CH₂—), and n-hexylene (-CH₂CH₂CH₂CH₂CH₂CH₂-)), C₁₋₆ heteroalkylene (e.g., C₁₋₆ heteroalkylene comprising 1-5 heteroatoms selected from N, O, and S), C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)), C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)), C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₅), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)), 3- to 12-membered heterocyclylene (e.g., heterocyclylene comprising one or two 3- to 8-membered rings and 1-5 heteroatoms selected from N, O, and S), C₆₋₁₀ arylene (e.g., phenylene or naphthylene), 5- to 10-membered heteroarylene (e.g., heteroarylene comprising one or two 5- or 6-membered rings and 1-5 heteroatoms selected from N, O, and S), —C(═O)—, —C(═O)N(R^{L})—, —C(═O)O)—, —N(R^{L})—, —O—, —S—, or —S(═O—, wherein the alkylene, alkenylene, carbocyclylene, heterocyclylene, arylene, or heteroarylene is optionally substituted with one or more R^{u}.

In certain embodiments, each L′ is independently C₁₋₆ alkylene, C₃₋₁₂ carbocyclylene, 3- to 12-membered heterocyclylene, —C(═O)—, —C(═O)N(R^{L})—, —C(═O)O—, —N(R^{L})—, or —O—, wherein the alkylene, carbocyclylene, or heterocyclylene is optionally substituted with one or more R^{u}.

In certain embodiments, each occurrence of R^{L} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃). 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{L} is independently hydrogen, C₁₋₆ alkyl, C₂₋₆ alkenyl, C₂₋₆ alkynyl, C₃₋₆ carbocyclyl, or 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{L} is independently hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, each occurrence of R^{L} is independently hydrogen, C₁₋₆ alkyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(-O)NR^{c}R^{d}, wherein the alkyl, carbocyclyl, or heterocyclyl is optionally substituted with one or more R^{u}.

In certain embodiments, 1 is 0. In certain embodiments, 1 is 1. In certain embodiments, 1 is 2. In certain embodiments, 1 is 3. In certain embodiments, 1 is 4. In certain embodiments, 1 is 5. In certain embodiments, 1 is 6. In certain embodiments, 1 is 7. In certain embodiments, 1 is 8. In certain embodiments, 1 is 9. In certain embodiments, 1 is 10.

In certain embodiments, L is of Formula I-3:

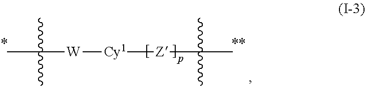

wherein:
* denotes attachment to T and ** denotes attachment to C;
W is absent; or
W is C₁₋₃ alkylene, —O—, —NR^{W}—, or —(C═O)—, wherein the alkylene is optionally substituted by one or more R^{u};
—Cy¹ is absent; or
—Cy¹ is 6-membered heteroarylene, C₆ arylene, C₃₋₁₂ carbocyclylene, or 3- to 12-membered heterocyclylene, wherein the arylene, heteroarylene, carbocyclylene, or heterocyclylene is optionally substituted by one or more R^{u};
Z′ is absent; or
each Z′ is independently C₁₋₃ alkylene, —O—, —NR^{W}—, —(C═O)—, C₃₋₁₂ carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted with one or more R^{u}; and
p is an integer selected from 0 to 8.

In certain embodiments, W is absent.

In certain embodiments, W is C₁₋₃ alkylene (e.g., methylene (-CH₂—), ethylene (-CH₂CH₂-), or n-propylene (-CH₂CH₂CH₂-)), —O—, —NR^{W}—, or —(C═O)—, wherein the alkylene is optionally substituted by one or more R^{u}.

In certain embodiments. Cy¹ is absent.

In certain embodiments, Cy¹ is C₆ arylene (i.e., phenylene), 6-membered heteroarylene (e.g., heteroarylene comprising one 6-membered ring and 1-4 heteroatoms selected from N, O, and S), C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₅), cyclooctenylene (C₈), bicyclo[2.2.1]heptanylene (C₇), bicyclo[2.2.2]octanylene (C₈), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)), or 3- to 12-membered heterocyclylene (e.g., heterocyclylene comprising one or two 3- to 8-membered rings and 1-5 heteroatoms selected from N, O, and S), wherein the arylene, heteroarylene, carbocyclylene, or heterocyclylene is optionally substituted by one or more R^{u}.

In certain embodiments. Cy¹ is 3- to 12-membered heterocyclylene selected from morpholinylene, piperidinylene, piperazinylene, 7-azaspiro[3.5]nonanylene, 2,7-diazaspiro[3.5]nonanylene, 2-azaspiro[3.5]nonanylene, 2,7-diazaspiro[3.5]nonanylene, 1-oxa-8-azaspiro[4.5] decenylene, 2-oxa-8-azaspiro[4.5] decenylene, 5-oxa-2-azaspiro[3,4]octanylene, 6-oxa-2-azaspiro[3,4]octanylene, 3,9-diazaspiro[5.5]undecanylene, 5-oxa-2-azaspiro[3.5]nonanylene, 1-oxa-9-azaspiro[5.5]undecanylene, 1-oxa-4,9-diazaspiro[5.5]undecanylene, 2,6-diazaspiro[3.3]heptanylene, 2-azaspiro[3.3]heptanylene, 1,5-dioxa-9-azaspiro[5.5]undecanylene, 1,4-dioxa-9-azaspiro[5.5]undecanylene, 5,9-dioxa-2-azaspiro[3.5]nonanylene, 5,8-dioxa-2-azaspiro[3.5]nonanylene, 6-oxa-2-azaspiro[3.5]nonanylene, 1-oxa-7-azaspiro[3.5]nonanylene, 5-oxa-2-azaspiro[3.6] decenylene, 5-oxa-2-azaspiro[3.6] decenylene, 5,9-dioxa-2-azaspiro[3.6] decenylene, 5,8-dioxa-2-azaspiro[3.6] decenylene, and 6,9-dioxa-2-azaspiro[3.6] decenylene, wherein the heterocyclylene is optionally substituted by one or more R^{u}.

In certain embodiments. Cy¹ is 3- to 12-membered heterocyclylene selected from

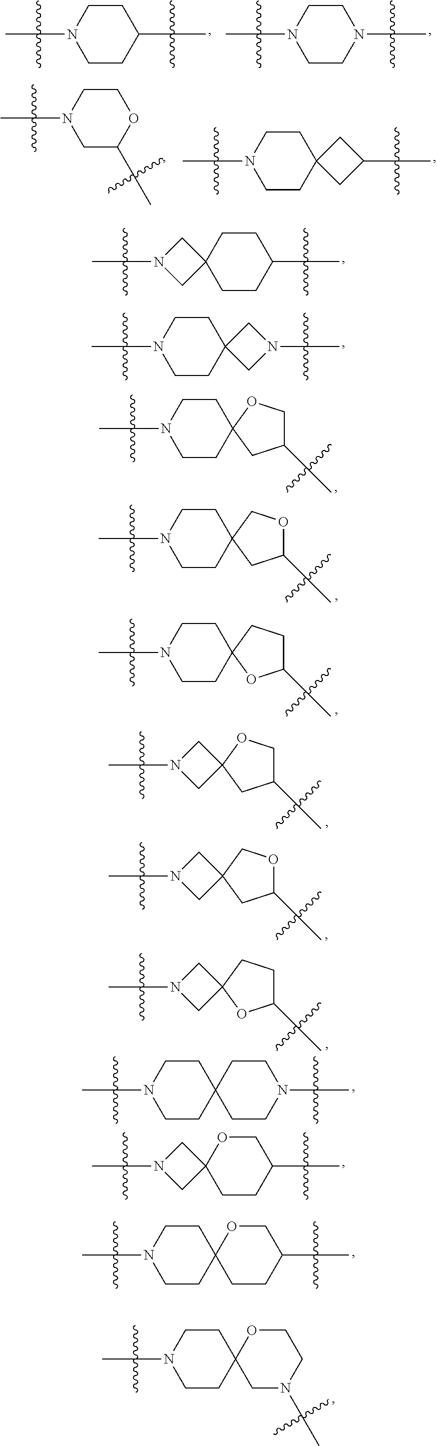

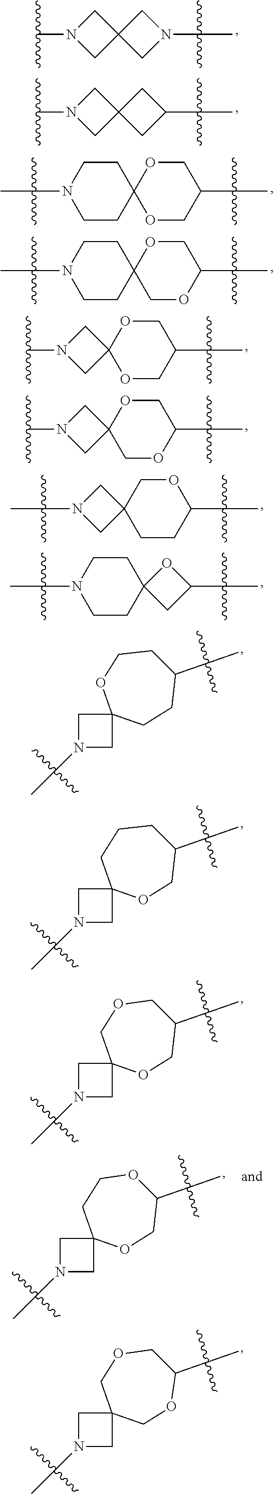

wherein the heterocyclylene is optionally substituted by one or more R^{u}.

In certain embodiments, Z′ is absent.

In certain embodiments, each Z′ is independently C₁₋₃ alkylene (e.g., methylene (-CH₂-), ethylene (-CH₂CH₂-), or n-propylene (-CH₂CH₂CH₂-)), —O—, —NR^{W}—, —(C═O)—, C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), or 3- to 12-membered heterocyclyl (e.g., heterocyclyl comprising one or two 3- to 8-membered rings and 1-5 heteroatoms selected from N, O, and S), wherein the alkylene, carbocyclylene, or heterocyclylene is optionally substituted by one or more R^{u}.

In certain embodiments, R^{W} is hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, p is 0. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 7. In certain embodiments, p is 8.

In certain embodiments, -[Z′] p- is-C(═O)—, C₁₋₆ alkylene, *-O—(C₁₋₆ alkylene)-, *-(C₁₋₆ alkylene)-(C(═O))—O—, *-(C₁₋₆ alkylene)-O—, *—C(═O)-(C₁₋₆ alkylene)-, *-(C₁₋₆ alkylene)-C(═O)—, 3-to 12-membered heterocyclylene, *—C(═O)-(3- to 12-membered heterocyclylene)-, *-(3- to 12-membered heterocyclylene)-C(═O)—, *-(3- to 12-membered heterocyclylene)-(C₁₋₆ alkylene)-, *-(C₁₋₆ alkylene)-(3- to 12-membered heterocyclylene)-, *-(C₁₋₆ alkylene)-(3- to 12-membered heterocyclylene)-(C₁₋₆ alkylene)-, *-(C₁₋₆ alkylene)-(3- to 12-membered heterocyclylene)-(C(═O))—, *-(C(═O))-(3- to 12-membered heterocyclylene)-(C₁₋₆ alkylene)-, *-(3- to 12-membered heterocyclylene)-(C₁₋₆ alkylene)-(C(═O))—, *-(C(═O))-(C₁₋₆ alkylene)-(3- to 12-membered heterocyclylene)-, *-(C₁₋₆ alkylene)-(C(═O))-(3- to 12-membered heterocyclylene)-, or *-(3- to 12-membered heterocyclylene)-(C(═O))-(C₁₋₆ alkylene)-, wherein the alkylene or heterocyclylene is optionally substituted by one or more R^{u}, and *denotes attachment to C.

In certain embodiments, -[Z′] p- is-C(═O)—, C₁₋₆ alkylene, *-(C₁₋₆ alkylene)-(C(═O))—O—, *-C(═O)-(C₁₋₆ alkylene)-, *-(C₁₋₆ alkylene)-C(═O)—, 3- to 12-membered heterocyclylene, *-(3- to 12-membered heterocyclylene)-(C₁₋₆ alkylene)-, *-(C(═O))-(3- to 12-membered heterocyclylene)-(C₁₋₆-alkylene)-, *-(C(═O))-(C₁₋₆ alkylene)-(3- to 12-membered heterocyclylene)-. *-(C₁₋₆ alkylene)-(C(═O))-(3- to 12-membered heterocyclylene)-, wherein the alkylene or heterocyclylene is optionally substituted by one or more R^{u}, and *denotes attachment to C.

In certain embodiments, L′ is W. In certain embodiments, L′ is Cy¹. In certain embodiments, L′ is Z′.

In certain embodiments, 1 is p. In certain embodiments, 1 is p+1. In certain embodiments, 1 is p+2.

In certain embodiments.
C is of Formula I-1-i

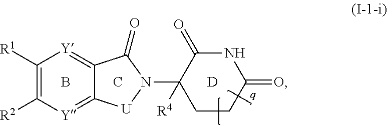

wherein:
R¹ and R², together with the intervening carbon atoms, form Ring A attached to L; or
R² and R³, together with the intervening carbon atoms, form Ring A attached to L; and Ring A is optionally substituted 7- to 16-membered fused hetcrocycle or optionally substituted 7-to 16-membered spiro heterocycle,
T is of Formula I-2:

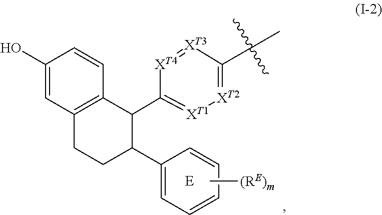

wherein:
each of X^{T1}, X^{T2}, X^{T3}, and X^{T4} is CRT, wherein
   i) XTI is C(OCH₃), X^{T3} is CF, and each of X^{T2} and X^{T4} is CH; or
   ii) X^{T1} is C(OCH₃), X^{T2} is CF, and each of X^{T3} and X^{T4} is CH, and
L is of Formula I-3:

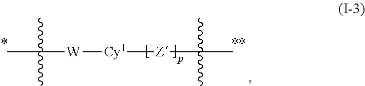

wherein:
W is absent;
Cy¹ is C₃₋₁₂ carbocyclylene or 3- to 12-membered heterocyclylene, wherein the carbocyclylene or heterocyclylene is optionally substituted by one or more R^{u};
each Z′ is independently C₁₋₃ alkylene optionally substituted by one or more R^{u}; and
p is an integer selected from 0 to 6.

In certain embodiments, Ring A is

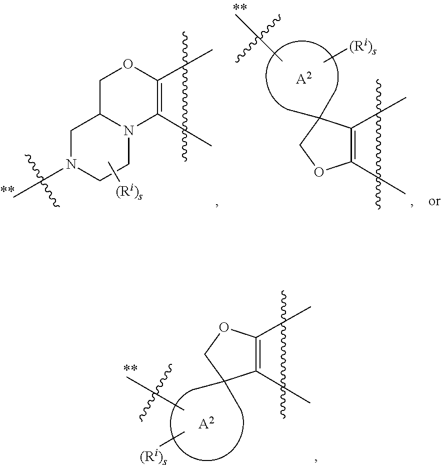

wherein ** denotes attachment to L; s is an integer selected from 0 to 8, as valency permits; and Ring A² is C₃₋₈ carbocycle or 3- to 8-membered heterocycle.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6- membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl (CI), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃).2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂₋₆ alkynyl, C₃₋₆ carbocyclyl, or 3- to 6-membered heterocyclyl.

In certain embodiments, each R^{b} is independently hydrogen, C₁₋₆ alkyl, C₃₋₆ carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₃₋₆ carbocyclyl, or 3- to 6-membered heterocyclyl, wherein the alkyl, carbocyclyl, or heterocyclyls optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, R^{b}, R^{c}, and R^{d} is independently and optionally substituted with one or more R^{z}.

In certain embodiments, R² is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, each R^{u} is independently oxo, halogen, —CN, —NO₂, —OH, —NH₂. C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino. methylpentylamino, methylhexylamino, ethyl-n-propylamino, ethyl-1-propylamino, ethyl-n-butylamino, ethyl-s-butylamino, ethyl-1-butylamino, ethyl--butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)). C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₅), bicyclo[2.2.1]heptanyl (C₇), bicyclo[2.2.2]octanyl (C₅), cyclononyl (C₉), cyclononynyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, -NO₂, —OH, —NH₂, C₁₋₆ alkyl, C₁₋₆ alkoxy. C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain embodiments, two geminal R^{u}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

Embodiments of the variables in any of the Formulae described herein, e.g., Formulae I and I′, as applicable, are described below. Any of the variables can be any moiety as described in the embodiments below. In addition, the combination of any moieties described for any of the variables, as applicable, with any moieties described for any of the remaining variables, is also contemplated.

Without wishing to be limited by this statement, while various options for variables are described herein, it is understood that the present disclosure intends to encompass operable embodiments having combinations of the options. The disclosure may be interpreted as excluding the non-operable embodiments caused by certain combinations of the options. For example, while various options for variables X and Z are described herein, the disclosure may be interpreted as excluding structures for non-operable compounds caused by certain combinations of the options (e.g., when two×or two Z are both nitrogen or both oxygen; or one of the two X or one of the two Z is nitrogen while the other is oxygen).

When a range of values is listed, each discrete value and sub-range within the range are also contemplated. For example, “C₁₋₆ alkyl” is intended to encompass, C₁, C₂, C₃, C₄, C₅, C₆, C₁-6, C₁₋₅, C₁₄, C₁₋₃, C₁₋₂, C₂₋₆, C₂₋₅, C₂₄, C₂₋₃, C₃₋₆, C₃₅, C₃₄, C₄₆, C₄₅, and C₅₆ alkyl.

In certain embodiments, the compound is selected from the compounds in Table X below, or a pharmaceutically acceptable salt thereof.

In certain embodiments, the compound is selected from the compounds in Table X below.

TABLE X

| Com- | | |
|---|---|---|
| pound | | |
| No. | Structure | Chemical Name |
| A99 | 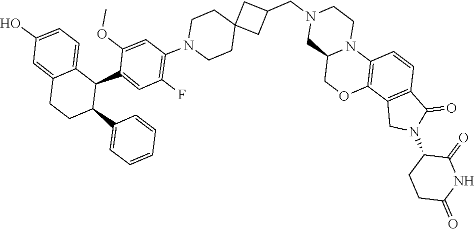 [SMILES: COc1cc(N2CCC3(CC2)CC(CN2CCN4c5ccc6c(c5OC[C@H]4C2)CN([C@H]2CCC(=O)NC2=O)C6=O)C3)c(F)cc1[C@@H]1c2ccc(O)cc2CC[C@@H]1c1ccccc1] | (S)-3-((R)-7-((7-(2-fluoro-4- ((1S,2S)-6-hydroxy-2-phenyl- 1,2,3,4-tetrahydronaphthalen-1- yl)-5-methoxyphenyl)-7- azaspiro[3.5]nonan-2- yl)methyl)-1-oxo- 1,3,5,5a,6,7,8,9-octahydro-2H- pyrazino[1′,2′:4,5][1,4] oxazino[2,3- e]isoindol-2-yl)piperidine-2,6- dione |
| A100 | 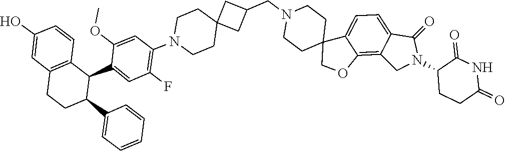 [SMILES: COc1cc(N2CCC3(CC2)CC(CN2CCC4(CC2)COc2c4ccc4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen-1- yl)-5-methoxyphenyl)-7- azaspiro[3.5]nonan-2- yl)methyl)-6-oxo-6,8-dihydro- 2H,7H-spiro[furo[2,3- e]isoindole-3,4′-piperidin]-7- yl)piperidine-2,6-dione |
| A129 | 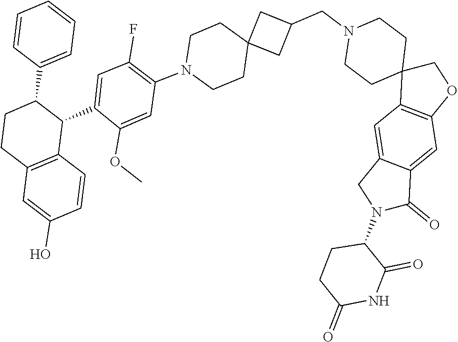 [SMILES: COc1cc(N2CCC3(CC2)CC(CN2CCC4(CC2)COc2cc5c(cc24)CN([C@H]2CCC(=O)NC2=O)C5=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen-1- yl)-5-methoxyphenyl)-7- azaspiro[3.5]nonan-2- yl)methyl)-7-oxo-5,7-dihydro- 2H,6H-spiro[furo[2,3- f]isoindole-3,4′-piperidin]-6- yl)piperidine-2,6-dione |
| A171 | 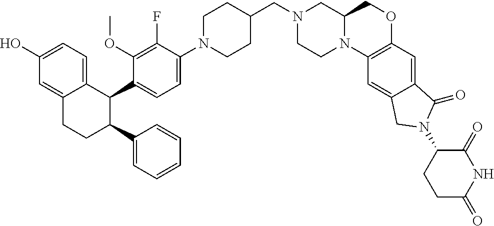 [SMILES: COc1c([C@@H]2c3ccc(O)cc3CC[C@@H]2c2ccccc2)ccc(N2CCC(CN3CCN4c5cc6c(cc5OC[C@@H]4C3)C(=O)N([C@H]3CCC(=O)NC3=O)C6)CC2)c1F] | (S)-3-((S)-3-((1-(2-fluoro-4- ((1S,2S)-6-hydroxy-2-phenyl- 1,2,3,4-tetrahydronaphthalen-1- yl)-3-methoxyphenyl)piperidin- 4-yl)methyl)-8-oxo- 1,2,3,4,4a,5,8,10-octahydro-9H- pyrazino[1′,2′:4,5][1,4] oxazino[2,3- f]isoindol-9-yl)piperidine-2,6- dione |
| B366 | 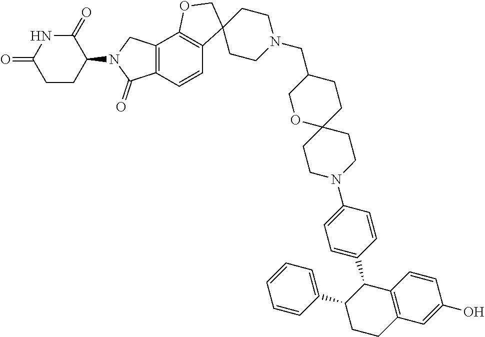 [SMILES: O=C1CC[C@H](N2Cc3c(ccc4c3OCC43CCN(CC4CCC5(CCN(c6ccc([C@@H]7c8ccc(O)cc8CC[C@@H]7c7ccccc7)cc6)CC5)OC4)CC3)C2=O)C(=O)N1] | (3S)-3-(1′-((9-(4-((1R,2S)-6- hydroxy-2-phenyl-1,2,3,4- tetrahydronaphthalen-1- yl)phenyl)-1-oxa-9- azaspiro[5.5]undecan-3- yl)methyl)-6-oxo-6,8-dihydro- 2H,7H-spiro[furo[2,3- e]isoindole-3,4′-piperidin]-7- yl)piperidine-2,6-dione |
| B366X | 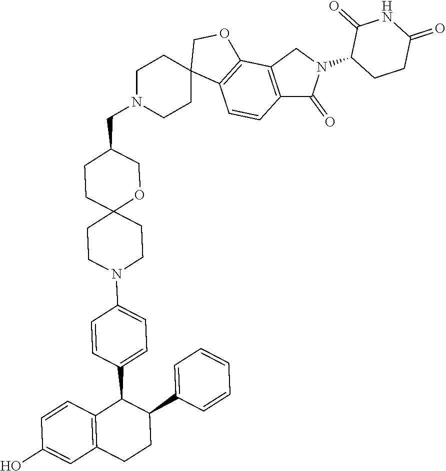 [SMILES: O=C1CC[C@H](N2Cc3c(ccc4c3OCC43CCN(C[C@@H]4CCC5(CCN(c6ccc([C@@H]7c8ccc(O)cc8CC[C@@H]7c7ccccc7)cc6)CC5)OC4)CC3)C2=O)C(=O)N1] | (S)-3-(1′-(((S)-9-(4-((1R,2S)-6- hydroxy-2-phenyl-1,2,3,4- tetrahydronaphthalen-1- yl)phenyl)-1-oxa-9- azaspiro[5.5]undecan-3- yl)methyl)-6-oxo-6,8-dihydro- 2H,7H-spiro[furo[2,3- e]isoindole-3,4′-piperidin]-7- yl)piperidine-2,6-dione |
| B366Y | 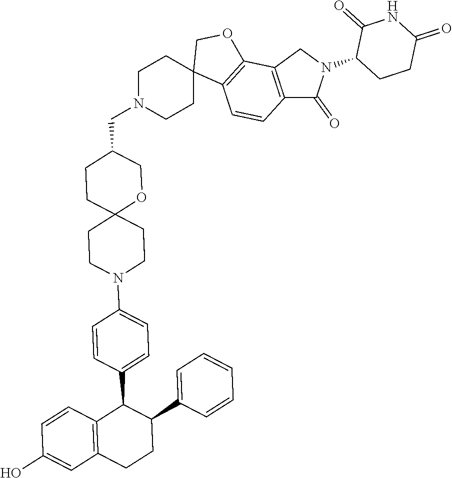 [SMILES: O=C1CC[C@H](N2Cc3c(ccc4c3OCC43CCN(C[C@H]4CCC5(CCN(c6ccc([C@@H]7c8ccc(O)cc8CC[C@@H]7c7ccccc7)cc6)CC5)OC4)CC3)C2=O)C(=O)N1] | (S)-3-(1′-(((R)-9-(4-((1R,2S)-6- hydroxy-2-phenyl-1,2,3,4- tetrahydronaphthalen-1- yl)phenyl)-1-oxa-9- azaspiro[5.5]undecan-3- yl)methyl)-6-oxo-6,8-dihydro- 2H,7H-spiro[furo[2,3- e]isoindole-3,4′-piperidin]-7- yl)piperidine-2,6-dione |
| B337X | 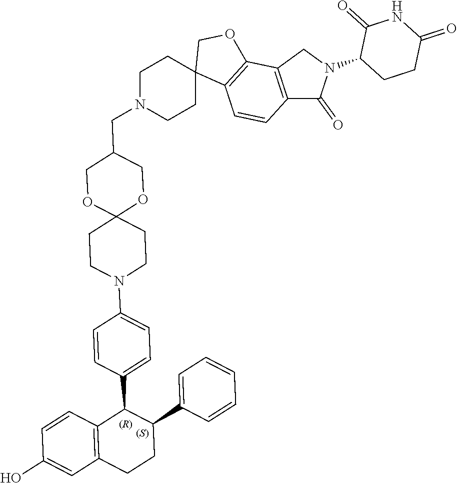 | (S)-3-(1′-((9-(4-((1R,2S)-6- hydroxy-2-phenyl-1,2,3,4- tetrahydronaphthalen-1- yl)phenyl)-1,5-dioxa-9- azaspiro[5.5]undecan-3- yl)methyl)-6-oxo-6,8-dihydro- 2H,7H-spiro[furo[2,3- e]isoindole-3,4′-piperidin]-7- yl)piperidine-2,6-dione |
| B306X | 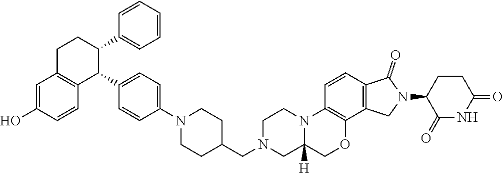 [SMILES: O=C1CC[C@H](N2Cc3c(ccc4c3OC[C@@H]3CN(CC5CCN(c6ccc([C@@H]7c8ccc(O)cc8CC[C@@H]7c7ccccc7)cc6)CC5)CCN43)C2=O)C(=O)N1] | (S)-3-((S)-7-((1-(4-((1R,2S)-6- hydroxy-2-phenyl-1,2,3,4- tetrahydronaphthalen-1- yl)phenyl)piperidin-4- yl)methyl)-1-oxo- 1,3,5,5a,6,7,8,9-octahydro-2H- pyrazino[1′,2′:4,5][1,4] oxazino[2,3- e]isoindol-2-yl)piperidine-2,6- dione |
| B308X | 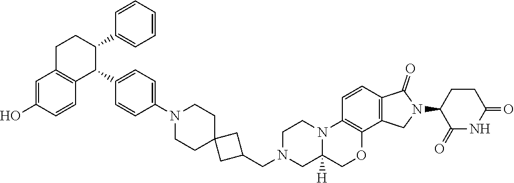 [SMILES: O=C1CC[C@H](N2Cc3c(ccc4c3OC[C@H]3CN(CC5CC6(CCN(c7ccc([C@@H]8c9ccc(O)cc9CC[C@@H]8c8ccccc8)cc7)CC6)C5)CCN43)C2=O)C(=O)N1] | (S)-3-((R)-7-((7-(4-((1R,2S)-6- hydroxy-2-phenyl-1,2,3,4- tetrahydronaphthalen-1- yl)phenyl)-7- azaspiro[3.5]nonan-2- yl)methyl)-1-oxo- 1,3,5,5a,6,7,8,9-octahydro-2H- pyrazino[1′,2′:4,5][1,4]oxazino[2,3- e]isoindol-2-yl)piperidine-2,6- dione |
| B330X | 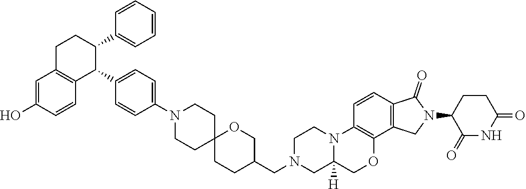 [SMILES: O=C1CC[C@H](N2Cc3c(ccc4c3OC[C@H]3CN(CC5CCC6(CCN(c7ccc([C@@H]8c9ccc(O)cc9CC[C@@H]8c8ccccc8)cc7)CC6)OC5)CCN43)C2=O)C(=O)N1] | (3S)-3-((5aR)-7-((9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-1-oxo- 1,3,5,5a,6,7,8,9-octahydro-2H- pyrazino[1′,2′:4,5][1,4] oxazino[2,3- e]isoindol-2-yl)piperidine-2,6- dione |
| A35 | 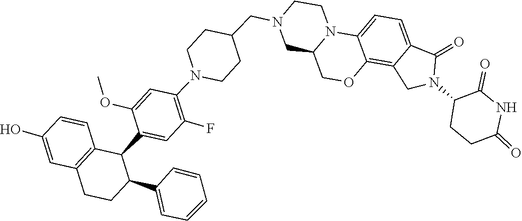 [SMILES: COc1cc(N2CCC(CN3CCN4c5ccc6c(c5OC[C@H]4C3)CN([C@H]3CCC(=O)NC3=O)C6=O)CC2)c(F)cc1[C@@H]1c2ccc(O)cc2CC[C@@H]1c1ccccc1] | (S)-3-((R)-7-((1-(2-fluoro-4- ((1S,2S)-6-hydroxy-2-phenyl- 1,2,3,4-tetrahydronaphthalen-1- yl)-5-methoxyphenyl)piperidin- 4-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6- dione |
| B135X | 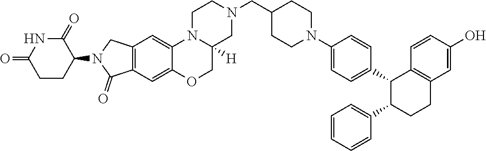 [SMILES: O=C1CC[C@H](N2Cc3cc4c(cc3C2=O)OC[C@@H]2CN(CC3CCN(c5ccc([C@@H]6c7ccc(O)cc7CC[C@@H]6c6ccccc6)cc5)CC3)CCN42)C(=O)N1] | (S)-3-((S)-3-((1-(4-((1R,2S)-6- hydroxy-2-phenyl-1,2,3,4- tetrahydronaphthalen-1- yl)phenyl)piperidin-4- yl)methyl)-8-oxo- 1,2,3,4,4a,5,8,10-octahydro-9H- pyrazino[1′,2′:4,5][1,4] oxazino[2,3- f]isoindol-9-yl)piperidine-2,6- dione |
| B211 | 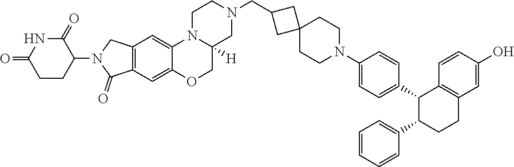 [SMILES: O=C1CCC(N2Cc3cc4c(cc3C2=O)OC[C@@H]2CN(CC3CC5(CCN(c6ccc([C@@H]7c8ccc(O)cc8CC[C@@H]7c7ccccc7)cc6)CC5)C3)CCN42)C(=O)N1] | 3-((S)-3-((7-(4-((1R,2S)-6- hydroxy-2-phenyl-1,2,3,4- tetrahydronaphthalen-1- yl)phenyl)-7- azaspiro[3.5]nonan-2- yl)methyl)-8-oxo- 1,2,3,4,4a,5,8,10-octahydro-9H- pyrazino[1′,2′:4,5][1,4] oxazino[2,3- f]isoindol-9-yl)piperidine-2,6- dione |
| B226X | 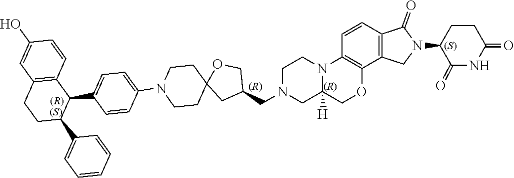 | (S)-3-((R)-7-(((R)-8-(4- ((1R,2S)-6-hydroxy-2-phenyl- 1,2,3,4-tetrahydronaphthalen-1- yl)phenyl)-1-oxa-8- azaspiro[4.5]decan-3-yl)methyl)- 1-oxo-1,3,5,5a,6,7,8,9- octahydro-2H- pyrazino[1′,2′:4,5][1,4] oxazino[2,3- e]isoindol-2-yl)piperidine-2,6- dione |
| B226Y | 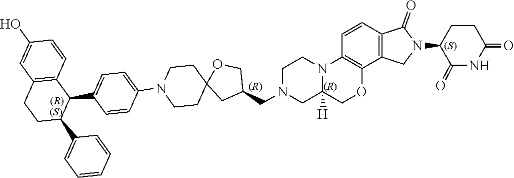 | (S)-3-((R)-7-(((S)-8-(4-((1R,2S)- 6-hydroxy-2-phenyl-1,2,3,4- tetrahydronaphthalen-1- yl)phenyl)-1-oxa-8- azaspiro[4.5]decan-3-yl)methyl)- 1-oxo-1,3,5,5a,6,7,8,9- octahydro-2H- pyrazino[1′,2′:4,5][1,4] oxazino[2,3- e]isoindol-2-yl)piperidine-2,6- dione |
| B227X | 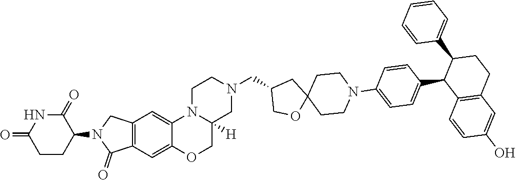 [SMILES: O=C1CC[C@H](N2Cc3cc4c(cc3C2=O)OC[C@@H]2CN(C[C@H]3COC5(CCN(c6ccc([C@@H]7c8ccc(O)cc8CC[C@@H]7c7ccccc7)cc6)CC5)C3)CCN42)C(=O)N1] | (S)-3-((S)-3-(((S)-8-(4-((1R,2S)- 6-hydroxy-2-phenyl-1,2,3,4- tetrahydronaphthalen-1- yl)phenyl)-1-oxa-8- azaspiro[4.5]decan-3-yl)methyl)- 8-oxo-1,2,3,4,4a,5,8,10- octahydro-9H- pyrazino[1′,2′:4,5][1,4] oxazino[2,3- f]isoindol-9-yl)piperidine-2,6- dione |
| B227Y | 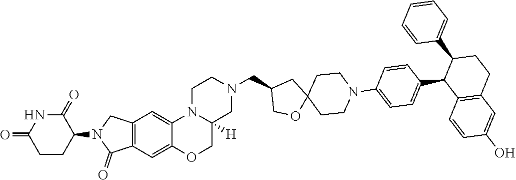 [SMILES: O=C1CC[C@H](N2Cc3cc4c(cc3C2=O)OC[C@@H]2CN(C[C@@H]3COC5(CCN(c6ccc([C@@H]7c8ccc(O)cc8CC[C@@H]7c7ccccc7)cc6)CC5)C3)CCN42)C(=O)N1] | (S)-3-((S)-3-(((R)-8-(4-((1R,2S)- 6-hydroxy-2-phenyl-1,2,3,4- tetrahydronaphthalen-1- yl)phenyl)-1-oxa-8- azaspiro[4.5]decan-3-yl)methyl)- 8-oxo-1,2,3,4,4a,5,8,10- octahydro-9H- pyrazino[1′,2′:4,5][1,4]oxazino[2,3- f]isoindol-9-yl)piperidine-2,6- dione |
| B229X | 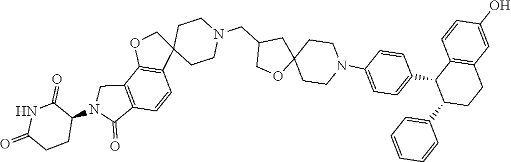 [SMILES: O=C1CC[C@H](N2Cc3c(ccc4c3OCC43CCN(CC4COC5(CCN(c6ccc([C@@H]7c8ccc(O)cc8CC[C@@H]7c7ccccc7)cc6)CC5)C4)CC3)C2=O)C(=O)N1] | (3S)-3-(1′-((8-(4-((1R,2S)-6- hydroxy-2-phenyl-1,2,3,4- tetrahydronaphthalen-1- yl)phenyl)-1-oxa-8- azaspiro[4.5]decan-3-yl)methyl)- 6-oxo-6,8-dihydro-2H,7H- spiro[furo[2,3-e]isoindole-3,4′- piperidin]-7-yl)piperidine-2,6- dione |
| A73 | 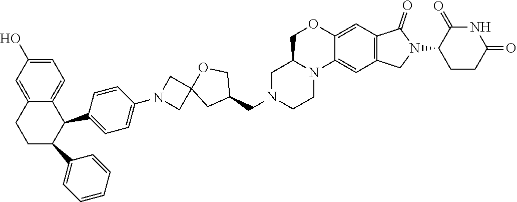 [SMILES: O=C1CC[C@H](N2Cc3cc4c(cc3C2=O)OC[C@@H]2CN(C[C@@H]3COC5(C3)CN(c3ccc([C@@H]6c7ccc(O)cc7CC[C@@H]6c6ccccc6)cc3)C5)CCN42)C(=O)N1] | (S)-3-((S)-3-(((R)-2-(4-((1R,2S)- 6-hydroxy-2-phenyl-1,2,3,4- tetrahydronaphthalen-1- yl)phenyl)-5-oxa-2- azaspiro[3.4]octan-7-yl)methyl)- 8-oxo-1,2,3,4,4a,5,8,10- octahydro-9H- pyrazino[1′,2′:4,5][1,4] oxazino[2,3- f]isoindol-9-yl)piperidine-2,6- dione |
| A40 | 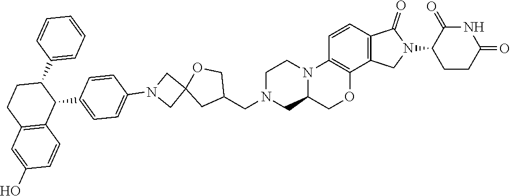 [SMILES: O=C1CC[C@H](N2Cc3c(ccc4c3OC[C@H]3CN(CC5COC6(C5)CN(c5ccc([C@@H]7c8ccc(O)cc8CC[C@@H]7c7ccccc7)cc5)C6)CCN43)C2=O)C(=O)N1] | (3S)-3-((5aR)-7-((2-(4-((1R,2S)- 6-hydroxy-2-phenyl-1,2,3,4- tetrahydronaphthalen-1- yl)phenyl)-5-oxa-2- azaspiro[3.4]octan-7-yl)methyl)- 1-oxo-1,3,5,5a,6,7,8,9- octahydro-2H- pyrazino[1′,2′:4,5][1,4]oxazino[2,3- e]isoindol-2-yl)piperidine-2,6- dione |
| A69 | 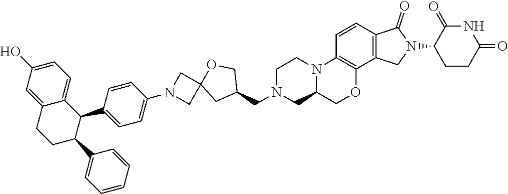 [SMILES: O=C1CC[C@H](N2Cc3c(ccc4c3OC[C@H]3CN(C[C@@H]5COC6(C5)CN(c5ccc([C@@H]7c8ccc(O)cc8CC[C@@H]7c7ccccc7)cc5)C6)CCN43)C2=O)C(=O)N1] | (S)-3-((R)-7-(((R)-2-(4- ((1R,2S)-6-hydroxy-2-phenyl- 1,2,3,4-tetrahydronaphthalen-1- yl)phenyl)-5-oxa-2- azaspiro[3.4]octan-7-yl)methyl)- 1-oxo-1,3,5,5a,6,7,8,9- octahydro-2H- pyrazino[1′,2′:4,5][1,4] oxazino[2,3- e]isoindol-2-yl)piperidine-2,6- dione |
| A76 | 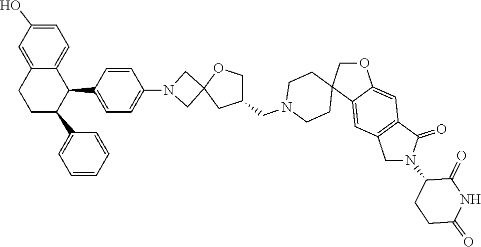 [SMILES: O=C1CC[C@H](N2Cc3cc4c(cc3C2=O)OCC42CCN(C[C@H]3COC4(C3)CN(c3ccc([C@@H]5c6ccc(O)cc6CC[C@@H]5c5ccccc5)cc3)C4)CC2)C(=O)N1] | (S)-3-(1′-(((S)-2-(4-((1R,2S)-6- hydroxy-2-phenyl-1,2,3,4- tetrahydronaphthalen-1- yl)phenyl)-5-oxa-2- azaspiro[3.4]octan-7-yl)methyl)- 7-oxo-5,7-dihydro-2H,6H- spiro[furo[2,3-f]isoindole-3,4′- piperidin]-6-yl)piperidine-2,6- dione |
| A77 | 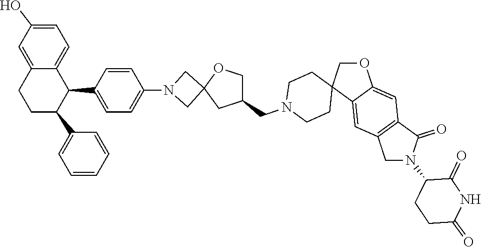 [SMILES: O=C1CC[C@H](N2Cc3cc4c(cc3C2=O)OCC42CCN(C[C@@H]3COC4(C3)CN(c3ccc([C@@H]5c6ccc(O)cc6CC[C@@H]5c5ccccc5)cc3)C4)CC2)C(=O)N1] | (S)-3-(1′-(((R)-2-(4-((1R,2S)-6- hydroxy-2-phenyl-1,2,3,4- tetrahydronaphthalen-1- yl)phenyl)-5-oxa-2- azaspiro[3.4]octan-7-yl)methyl)- 7-oxo-5,7-dihydro-2H,6H- spiro[furo[2,3-f]isoindole-3,4′- piperidin]-6-yl)piperidine-2,6- dione |
| A94 | 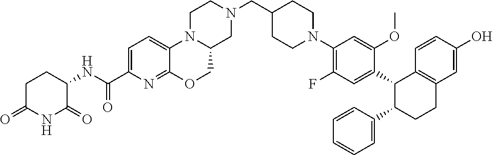 [SMILES: COc1cc(N2CCC(CN3CCN4c5ccc(C(=O)N[C@H]6CCC(=O)NC6=O)nc5OC[C@H]4C3)CC2)c(F)cc1[C@@H]1c2ccc(O)cc2CC[C@@H]1c1ccccc1] | (R)-N-((S)-2,6-dioxopiperidin-3- yl)-3-((1-(2-fluoro-4-((1S,2S)-6- hydroxy-2-phenyl-1,2,3,4- tetrahydronaphthalen-1-yl)-5- methoxyphenyl)piperidin-4- yl)methyl)-1,2,3,4,4a,5- hexahydropyrazino[1,2- d]pyrido[2,3-b][1,4]oxazine-8- carboxamide |
| A112 | 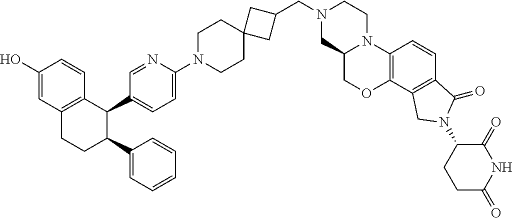 [SMILES: O=C1CC[C@H](N2Cc3c(ccc4c3OC[C@H]3CN(CC5CC6(CCN(c7ccc([C@@H]8c9ccc(O)cc9CC[C@@H]8c8ccccc8)cn7)CC6)C5)CCN43)C2=O)C(=O)N1] | (S)-3-((R)-7-((7-(5-((1S,2S)-6- hydroxy-2-phenyl-1,2,3,4- tetrahydronaphthalen-1- yl)pyridin-2-yl)-7- azaspiro[3.5]nonan-2- yl)methyl)-1-oxo- 1,3,5,5a,6,7,8,9-octahydro-2H- pyrazino[1′,2′:4,5][1,4] oxazino[2,3- e]isoindol-2-yl)piperidine-2,6- dione |
| A121 | 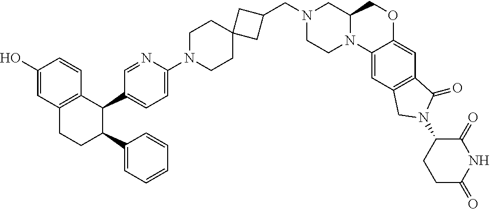 [SMILES: O=C1CC[C@H](N2Cc3cc4c(cc3C2=O)OC[C@@H]2CN(CC3CC5(CCN(c6ccc([C@@H]7c8ccc(O)cc8CC[C@@H]7c7ccccc7)cn6)CC5)C3)CCN42)C(=O)N1] | (S)-3-((S)-3-((7-(5-((1S,2S)-6- hydroxy-2-phenyl-1,2,3,4- tetrahydronaphthalen-1- yl)pyridin-2-yl)-7- azaspiro[3.5]nonan-2- yl)mcthyl)-8-oxo- 1,2,3,4,4a,5,8,10-octahydro-9H- pyrazino[1′,2′:4,5][1,4] oxazino[2,3- f]isoindol-9-yl)piperidine-2,6- dione |
| A130 | 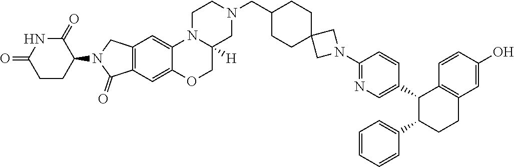 [SMILES: O=C1CC[C@H](N2Cc3cc4c(cc3C2=O)OC[C@@H]2CN(CC3CCC5(CC3)CN(c3ccc([C@@H]6c7ccc(O)cc7CC[C@@H]6c6ccccc6)cn3)C5)CCN42)C(=O)N1] | (S)-3-((S)-3-((2-(5-((1S,2S)-6- hydroxy-2-phenyl-1,2,3,4- tetrahydronaphthalen-1- yl)pyridin-2-yl)-2- azaspiro[3.5]nonan-7- yl)methyl)-8-oxo- 1,2,3,4,4a,5,8,10-octahydro-9H- pyrazino[1′,2′:4,5][1,4] oxazino[2,3- f]isoindol-9-yl)piperidine-2,6- dione |
| A131 | 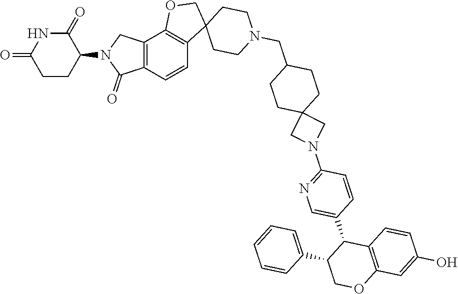 [SMILES: O=C1CC[C@H](N2Cc3c(ccc4c3OCC43CCN(CC4CCC5(CC4)CN(c4ccc([C@@H]6c7ccc(O)cc7OC[C@@H]6c6ccccc6)cn4)C5)CC3)C2=O)C(=O)N1] | (S)-3-(1′-((2-(5-((1S,2S)-6- hydroxy-2-phenyl-1,2,3,4- tetrahydronaphthalen-1- yl)pyridin-2-yl)-2- azaspiro[3.5]nonan-7- yl)methyl)-6-oxo-6,8-dihydro- 2H,7H-spiro[furo[2,3- e]isoindole-3,4′-piperidin]-7- yl)piperidine-2,6-dione |
| A133 | 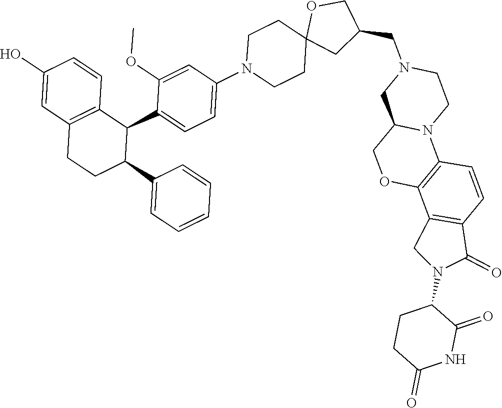 [SMILES: COc1cc(N2CCC3(CC2)C[C@H](CN2CCN4c5ccc6c(c5OC[C@H]4C2)CN([C@H]2CCC(=O)NC2=O)C6=O)CO3)ccc1[C@@H]1c2ccc(O)cc2CC[C@@H]1c1ccccc1] | (S)-3-((R)-7-(((R)-8-(4-((1S,2S)- 6-hydroxy-2-phenyl-1,2,3,4- tetrahydronaphthalen-1-yl)-3- methoxyphenyl)-1-oxa-8- azaspiro[4.5]decan-3-yl)methyl)- 1-oxo-1,3,5,5a,6,7,8,9- octahydro-2H- pyrazino[1′,2′:4,5][1,4] oxazino[2,3- e]isoindol-2-yl)piperidine-2,6- dione |
| A134 | 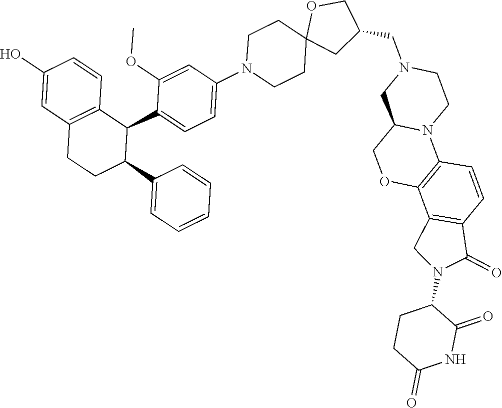 [SMILES: COc1cc(N2CCC3(CC2)C[C@@H](CN2CCN4c5ccc6c(c5OC[C@H]4C2)CN([C@H]2CCC(=O)NC2=O)C6=O)CO3)ccc1[C@@H]1c2ccc(O)cc2CC[C@@H]1c1ccccc1] | (S)-3-((R)-7-(((S)-8-(4-((1S,2S)- 6-hydroxy-2-phenyl-1,2,3,4- tetrahydronaphthalen-1-yl)-3- methoxyphenyl)-1-oxa-8- azaspiro[4.5]decan-3-yl)methyl)- 1-oxo-1,3,5,5a,6,7,8,9- octahydro-2H- pyrazino[1′,2′:4,5][1,4] oxazino[2,3- e]isoindol-2-yl)piperidine-2,6- dione |
| A135 | 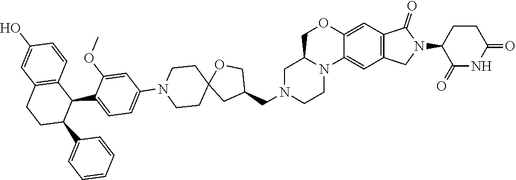 [SMILES: COc1cc(N2CCC3(CC2)C[C@H](CN2CCN4c5cc6c(cc5OC[C@@H]4C2)C(=O)N([C@H]2CCC(=O)NC2=O)C6)CO3)ccc1[C@@H]1c2ccc(O)cc2CC[C@@H]1c1ccccc1] | (S)-3-((S)-3-(((R)-8-(4-((1S,2S)- 6-hydroxy-2-phenyl-1,2,3,4- tetrahydronaphthalen-1-yl)-3- methoxyphenyl)-1-oxa-8- azaspiro[4.5]decan-3-yl)methyl)- 8-oxo-1,2,3,4,4a,5,8,10- octahydro-9H- pyrazino[1′,2′:4,5][1,4] oxazino[2,3- f]isoindol-9-yl)piperidine-2,6- dione |
| A136 | 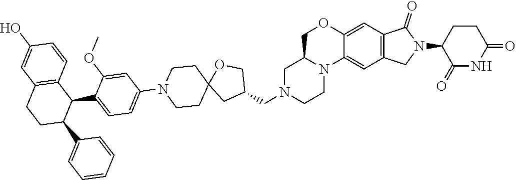 [SMILES: COc1cc(N2CCC3(CC2)C[C@@H](CN2CCN4c5cc6c(cc5OC[C@@H]4C2)C(=O)N([C@H]2CCC(=O)NC2=O)C6)CO3)ccc1[C@@H]1c2ccc(O)cc2CC[C@@H]1c1ccccc1] | (S)-3-((S)-3-(((S)-8-(4-((1S,2S)- 6-hydroxy-2-phenyl-1,2,3,4- tetrahydronaphthalen-1-yl)-3- methoxyphenyl)-1-oxa-8- azaspiro[4.5]decan-3-yl)methyl)- 8-oxo-1,2,3,4,4a,5,8,10- octahydro-9H- pyrazino[1′,2′:4,5][1,4]oxazino[2,3- f]isoindol-9-yl)piperidine-2,6- dione |
| A140 | 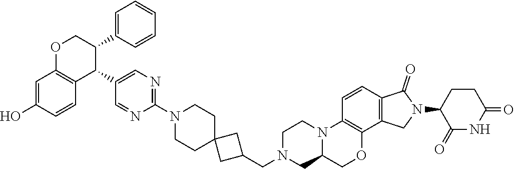 [SMILES: O=C1CC[C@H](N2Cc3c(ccc4c3OC[C@H]3CN(CC5CC6(CCN(c7ncc([C@@H]8c9ccc(O)cc9OC[C@@H]8c8ccccc8)cn7)CC6)C5)CCN43)C2=O)C(=O)N1] | (S)-3-((R)-7-((7-(5-((1S,2S)-6- hydroxy-2-phenyl-1,2,3,4- tetrahydronaphthalen-1- yl)pyrimidin-2-yl)-7- azaspiro[3.5]nonan-2- yl)methyl)-1-oxo- 1,3,5,5a,6,7,8,9-octahydro-2H- pyrazino[1′,2′:4,5][1,4] oxazino[2,3- e]isoindol-2-yl)piperidine-2,6- dione |
| A154 | 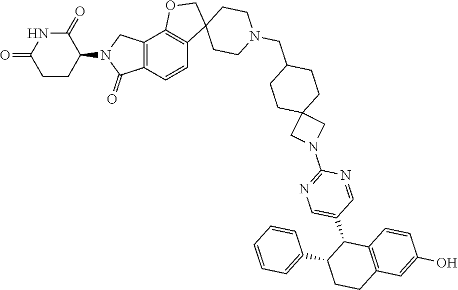 [SMILES: O=C1CC[C@H](N2Cc3c(ccc4c3OCC43CCN(CC4CCC5(CC4)CN(c4ncc([C@@H]6c7ccc(O)cc7CC[C@@H]6c6ccccc6)cn4)C5)CC3)C2=O)C(=O)N1] | (S)-3-(1′-((2-(5-((1S,2S)-6- hydroxy-2-phenyl-1,2,3,4- tetrahydronaphthalen-1- yl)pyrimidin-2-yl)-2- azaspiro[3.5]nonan-7- yl)methyl)-6-oxo-6,8-dihydro- 2H,7H-spiro[furo[2,3- e]isoindole-3,4′-piperidin]-7- yl)piperidine-2,6-dione |
| A163 | 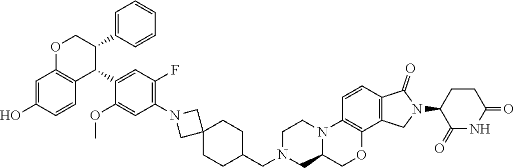 [SMILES: COc1cc(N2CC3(CCC(CN4CCN5c6ccc7c(c6OC[C@H]5C4)CN([C@H]4CCC(=O)NC4=O)C7=O)CC3)C2)c(F)cc1[C@@H]1c2ccc(O)cc2OC[C@@H]1c1ccccc1] | (S)-3-((R)-7-((2-(2-fluoro-4- ((1S,2S)-6-hydroxy-2-phenyl- 1,2,3,4-tetrahydronaphthalen-1- yl)-5-methoxyphenyl)-2- azaspiro[3.5]nonan-7- yl)methyl)-1-oxo- 1,3,5,5a,6,7,8,9-octahydro-2H- pyrazino[1′,2′:4,5][1,4] oxazino[2,3- e]isoindol-2-yl)piperidine-2,6- dione |
| A164 | 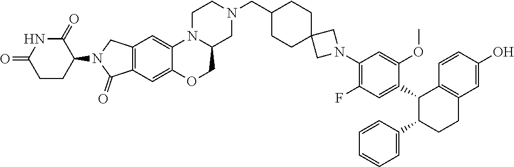 [SMILES: COc1cc(N2CC3(CCC(CN4CCN5c6cc7c(cc6OC[C@@H]5C4)C(=O)N([C@H]4CCC(=O)NC4=O)C7)CC3)C2)c(F)cc1[C@@H]1c2ccc(O)cc2CC[C@@H]1c1ccccc1] | (S)-3-((S)-3-((2-(2-fluoro-4- ((1S,2S)-6-hydroxy-2-phenyl- 1,2,3,4-tetrahydronaphthalen-1- yl)-5-methoxyphenyl)-2- azaspiro[3.5]nonan-7- yl)methyl)-8-oxo- 1,2,3,4,4a,5,8,10-octahydro-9H- pyrazino[1′,2′:4,5][1,4] oxazino[2,3- f]isoindol-9-yl)piperidine-2,6- dione |
| A91 | 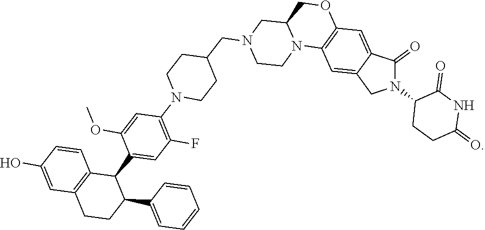 [SMILES: COc1cc(N2CCC(CN3CCN4c5cc6c(cc5OC[C@@H]4C3)C(=O)N([C@H]3CCC(=O)NC3=O)C6)CC2)c(F)cc1[C@@H]1c2ccc(O)cc2CC[C@@H]1c1ccccc1] | (S)-3-((S)-3-((1-(2-fluoro-4- ((1S,2S)-6-hydroxy-2-phenyl- 1,2,3,4-tetrahydronaphthalen-1- yl)-5-methoxyphenyl)piperidin- 4-yl)methyl)-8-oxo- 1,2,3,4,4a,5,8,10-octahydro-9H- pyrazino[1′,2′:4,5][1,4] oxazino[2,3- f]isoindol-9-yl)piperidine-2,6- dione |
| A173 | 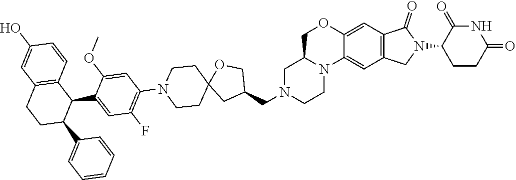 [SMILES: COc1cc(N2CCC3(CC2)C[C@H](CN2CCN4c5cc6c(cc5OC[C@@H]4C2)C(=O)N([C@H]2CCC(=O)NC2=O)C6)CO3)c(F)cc1[C@@H]1c2ccc(O)cc2CC[C@@H]1c1ccccc1] | (S)-3-((S)-3-(((R)-8-(2-fluoro-4- ((1S,2S)-6-hydroxy-2-phenyl- 1,2,3,4-tetrahydronaphthalen-1- yl)-5-methoxyphenyl)-1-oxa-8- azaspiro[4.5]decan-3-yl)methyl)- 8-oxo-1,2,3,4,4a,5,8,10- octahydro-9H- pyrazino[1′,2′:4,5][1,4] oxazino[2,3- f]isoindol-9-yl)piperidine-2,6- dione |
| A174 | 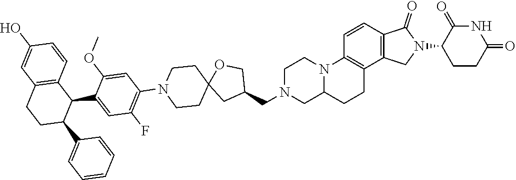 [SMILES: COc1cc(N2CCC3(CC2)C[C@H](CN2CCN4c5ccc6c(c5CCC4C2)CN([C@H]2CCC(=O)NC2=O)C6=O)CO3)c(F)cc1[C@@H]1c2ccc(O)cc2CC[C@@H]1c1ccccc1] | (S)-3-((R)-7-(((R)-8-(2-fluoro-4- ((1S,2S)-6-hydroxy-2-phenyl- 1,2,3,4-tetrahydronaphthalen-1- yl)-5-methoxyphenyl)-1-oxa-8- azaspiro[4.5]decan-3-yl)methyl)- 1-oxo-1,3,5,5a,6,7,8,9- octahydro-2H- pyrazino[1′,2′:4,5][1,4] oxazino[2,3- e]isoindol-2-yl)piperidine-2,6- dione |
| A175 | 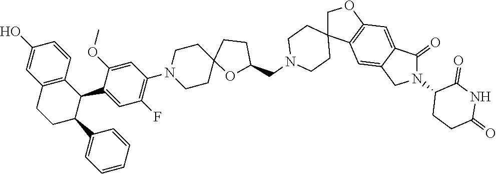 [SMILES: COc1cc(N2CCC3(CC[C@@H](CN4CCC5(CC4)COc4cc6c(cc45)CN([C@H]4CCC(=O)NC4=O)C6=O)O3)CC2)c(F)cc1[C@@H]1c2ccc(O)cc2CC[C@@H]1c1ccccc1] | (S)-3-(1′-(((R)-8-(2-fluoro-4- ((1S,2S)-6-hydroxy-2-phenyl- 1,2,3,4-tetrahydronaphthalen-1- yl)-5-methoxyphenyl)-1-oxa-8- azaspiro[4.5]decan-3-yl)methyl)- 7-oxo-5,7-dihydro-2H,6H- spiro[furo[2,3-f]isoindole-3,4′- piperidin]-6-yl)piperidine-2,6- dione |
| A176 | 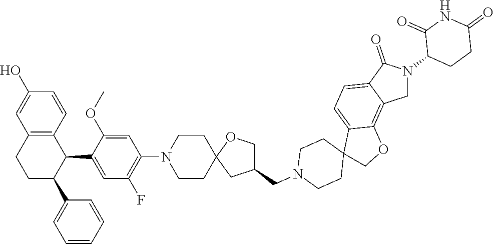 [SMILES: COc1cc(N2CCC3(CC2)C[C@H](CN2CCC4(CC2)COc2c4ccc4c2CN([C@H]2CCC(=O)NC2=O)C4=O)CO3)c(F)cc1[C@@H]1c2ccc(O)cc2CC[C@@H]1c1ccccc1] | (S)-3-(1′-(((R)-8-(2-fluoro-4- ((1S,2S)-6-hydroxy-2-phenyl- 1,2,3,4-tetrahydronaphthalen-1- yl)-5-methoxyphenyl)-1-oxa-8- azaspiro[4.5]decan-3-yl)methyl)- 6-oxo-6,8-dihydro-2H,7H- spiro[furo[2,3-c]isoindole-3,4′- piperidin]-7-yl)piperidine-2,6- dione |
| A203 | 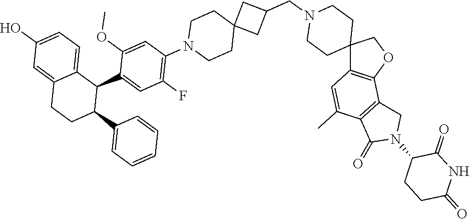 [SMILES: COc1cc(N2CCC3(CC2)CC(CN2CCC4(CC2)COc2c4cc(C)c4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen- 1-yl)-5-methoxyphenyl)-7- azaspiro[3.5]nonan-2- yl)methyl)-5-methyl-6-oxo- 6,8-dihydro-2H,7H- spiro[furo[2,3-e]isoindole- 3,4′-piperidin]-7- yl)piperidine-2,6-dione |
| A209 | 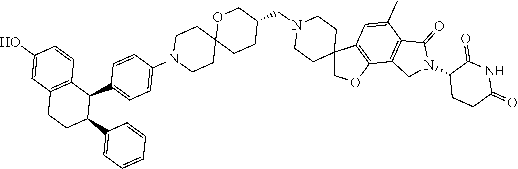 [SMILES: Cc1cc2c(c3c1C(=O)N([C@H]1CCC(=O)NC1=O)C3)OCC21CCN(C[C@@H]2CCC3(CCN(c4ccc([C@@H]5c6ccc(O)cc6CC[C@@H]5c5ccccc5)cc4)CC3)OC2)CC1] | (S)-3-(1′-(((S)-9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-5-methyl-6-oxo- 6,8-dihydro-2H,7H- spiro[furo[2,3-e]isoindole- 3,4′-piperidin]-7- yl)piperidine-2,6-dione |
| A210 | 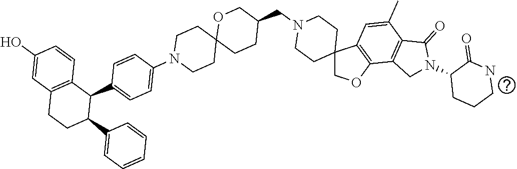 [SMILES: Cc1cc2c(c3c1C(=O)N([C@H]1CCCNC1=O)C3)OCC21CCN(C[C@H]2CCC3(CCN(c4ccc([C@@H]5c6ccc(O)cc6CC[C@@H]5c5ccccc5)cc4)CC3)OC2)CC1] | (S)-3-(1′-(((R)-9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-5-methyl-6-oxo- 6,8-dihydro-2H,7H- spiro[furo[2,3-e]isoindole- 3,4′-piperidin]-7- yl)piperidine-2,6-dione |
| A222 | 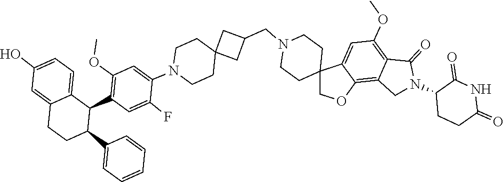 [SMILES: COc1cc(N2CCC3(CC2)CC(CN2CCC4(CC2)COc2c4cc(OC)c4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen- 1-yl)-5-methoxyphenyl)-7- azaspiro[3.5]nonan-2- yl)methyl)-5-methoxy-6-oxo- 6,8-dihydro-2H,7H- spiro[furo[2,3-e]isoindole- 3,4′-piperidin]-7- yl)piperidine-2,6-dione |
| A223 | 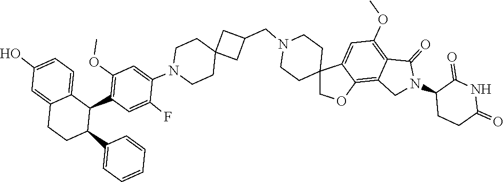 [SMILES: COc1cc(N2CCC3(CC2)CC(CN2CCC4(CC2)COc2c4cc(OC)c4c2CN([C@@H]2CCC(=O)NC2=O)C4=O)C3)c(F)cc1[C@@H]1c2ccc(O)cc2CC[C@@H]1c1ccccc1] | (R)-3-(1′-((7-(2-fluoro-4- ((1S,2S)-6-hydroxy-2-phenyl- 1,2,3,4-tetrahydronaphthalen- 1-yl)-5-methoxyphenyl)-7- azaspiro[3.5]nonan-2- yl)methyl)-5-methoxy-6-oxo- 6,8-dihydro-2H,7H- spiro[furo[2,3-e]isoindole- 3,4′-piperidin]-7- yl)piperidine-2,6-dione |
| A225 | 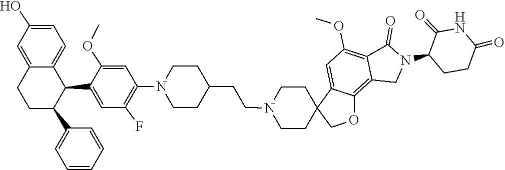 [SMILES: COc1cc(N2CCC(CCN3CCC4(CC3)COc3c4cc(OC)c4c3CN([C@@H]3CCC(=O)NC3=O)C4=O)CC2)c(F)cc1[C@@H]1c2ccc(O)cc2CC[C@@H]1c1ccccc1] | (R)-3-(1′-(2-(1-(2-fluoro-4- ((1S,2S)-6-hydroxy-2-phenyl- 1,2,3,4-tetrahydronaphthalen- 1-yl)-5- methoxyphenyl)piperidin-4- yl)ethyl)-5-methoxy-6-oxo- 6,8-dihydro-2H,7H- spiro[furo[2,3-e]isoindole- 3,4′-piperidin]-7- yl)piperidine-2,6-dione |
| A226 | 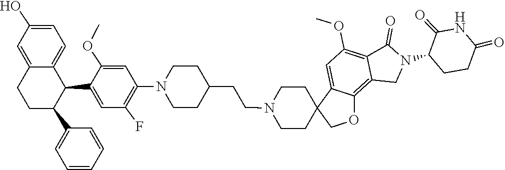 [SMILES: COc1cc(N2CCC(CCN3CCC4(CC3)COc3c4cc(OC)c4c3CN([C@H]3CCC(=O)NC3=O)C4=O)CC2)c(F)cc1[C@@H]1c2ccc(O)cc2CC[C@@H]1c1ccccc1] | (S)-3-(1′-(2-(1-(2-fluoro-4- ((1S,2S)-6-hydroxy-2-phenyl- 1,2,3,4-tetrahydronaphthalen- 1-yl)-5- methoxyphenyl)piperidin-4- yl)ethyl)-5-methoxy-6-oxo- 6,8-dihydro-2H,7H- spiro[furo[2,3-e]isoindole- 3,4′-piperidin]-7- yl)piperidine-2,6-dione |
| A227 | 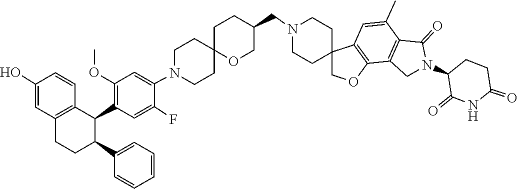 [SMILES: COc1cc(N2CCC3(CC[C@@H](CN4CCC5(CC4)COc4c5cc(C)c5c4CN([C@H]4CCC(=O)NC4=O)C5=O)CO3)CC2)c(F)cc1[C@@H]1c2ccc(O)cc2CC[C@@H]1c1ccccc1] | (S)-3-(1′-(((S)-9-(2-fluoro-4- ((1S,2S)-6-hydroxy-2-phenyl- 1,2,3,4-tetrahydronaphthalen- 1-yl)-5-methoxyphenyl)-1- oxa-9-azaspiro[5.5]undecan- 3-yl)methyl)-5-methyl-6-oxo- 6,8-dihydro-2H,7H- spiro[furo[2,3-e]isoindole- 3,4′-piperidin]-7- yl)piperidine-2,6-dione |
| A230 | 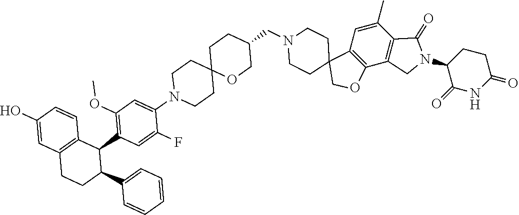 [SMILES: COc1cc(N2CCC3(CC[C@H](CN4CCC5(CC4)COc4c5cc(C)c5c4CN([C@H]4CCC(=O)NC4=O)C5=O)CO3)CC2)c(F)cc1[C@@H]1c2ccc(O)cc2CC[C@@H]1c1ccccc1] | (S)-3-(1′-(((R)-9-(2-fluoro-4- ((1S,2S)-6-hydroxy-2-phenyl- 1,2,3,4-tetrahydronaphthalen- 1-yl)-5-methoxyphenyl)-1- oxa-9-azaspiro[5.5]undecan- 3-yl)methyl)-5-methyl-6-oxo- 6,8-dihydro-2H,7H- spiro[furo[2,3-e]isoindole- 3,4′-piperidin]-7- yl)piperidine-2,6-dione |
| A231 | 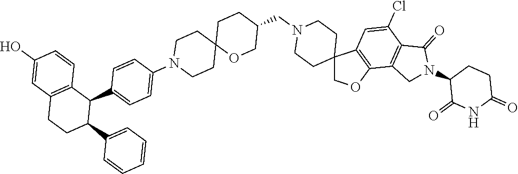 [SMILES: O=C1CC[C@H](N2Cc3c4c(cc(Cl)c3C2=O)C2(CCN(C[C@H]3CCC5(CCN(c6ccc([C@@H]7c8ccc(O)cc8CC[C@@H]7c7ccccc7)cc6)CC5)OC3)CC2)CO4)C(=O)N1] | (S)-3-(5-chloro-1′-(((R)-9-(4- ((1R,2S)-6-hydroxy-2-phenyl- 1,2,3,4-tetrahydronaphthalen- 1-yl)phenyl)-1-oxa-9- azaspiro[5.5]undecan-3- yl)methyl)-6-oxo-6,8- dihydro-2H,7H- spiro[furo[2,3-e]isoindole- 3,4′-pipcridin]-7- yl)piperidine-2,6-dione |
| A232 | 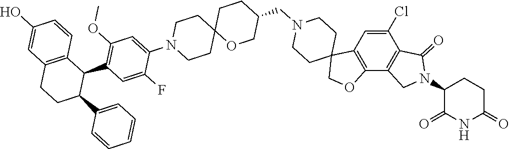 [SMILES: COc1cc(N2CCC3(CC[C@H](CN4CCC5(CC4)COc4c5cc(Cl)c5c4CN([C@H]4CCC(=O)NC4=O)C5=O)CO3)CC2)c(F)cc1[C@@H]1c2ccc(O)cc2CC[C@@H]1c1ccccc1] | (S)-3-(5-chloro-1′-(((R)-9-(2- fluoro-4-((1S,2S)-6-hydroxy- 2-phenyl-1,2,3,4- tetrahydronaphthalen-1-yl)-5- methoxyphenyl)-1-oxa-9- azaspiro[5.5]undecan-3- yl)methyl)-6-oxo-6,8- dihydro-2H,7H- spiro[furo[2,3-e]isoindole- 3,4′-piperidin]-7- yl)piperidine-2,6-dione |
| A233 | 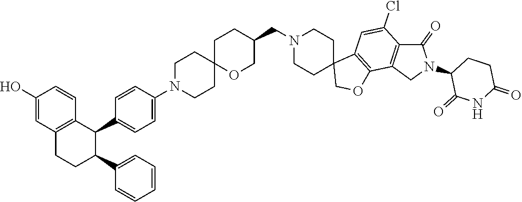 [SMILES: O=C1CC[C@H](N2Cc3c4c(cc(Cl)c3C2=O)C2(CCN(C[C@@H]3CCC5(CCN(c6ccc([C@@H]7c8ccc(O)cc8CC[C@@H]7c7ccccc7)cc6)CC5)OC3)CC2)CO4)C(=O)N1] | (S)-3-(5-chloro-1′-(((S)-9-(4- ((1R,2S)-6-hydroxy-2-phenyl- 1,2,3,4-tetrahydronaphthalen- 1-yl)phenyl)-1-oxa-9- azaspiro[5.5]undecan-3- yl)methyl)-6-oxo-6,8- dihydro-2H,7H- spiro[furo[2,3-e]isoindole- 3,4′-piperidin]-7- yl)piperidine-2,6-dione |
| A234 | 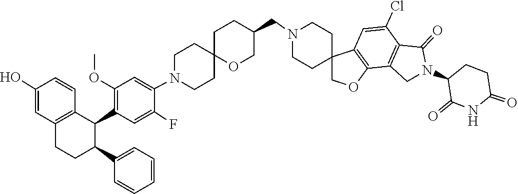 [SMILES: COc1cc(N2CCC3(CC[C@@H](CN4CCC5(CC4)COc4c5cc(Cl)c5c4CN([C@H]4CCC(=O)NC4=O)C5=O)CO3)CC2)c(F)cc1[C@@H]1c2ccc(O)cc2CC[C@@H]1c1ccccc1] | (S)-3-(5-chloro-1′-(((S)-9-(2- fluoro-4-((1S,2S)-6-hydroxy- 2-phenyl-1,2,3,4- tetrahydronaphthalen-1-yl)-5- methoxyphenyl)-1-oxa-9- azaspiro[5.5]undecan-3- yl)methyl)-6-oxo-6,8- dihydro-2H,7H- spiro[furo[2,3-e]isoindole- 3,4′-piperidin]-7- yl)piperidine-2,6-dione |
| A235 | 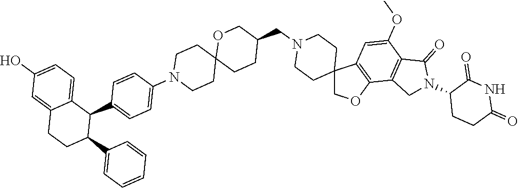 [SMILES: COc1cc2c(c3c1C(=O)N([C@H]1CCC(=O)NC1=O)C3)OCC21CCN(C[C@H]2CCC3(CCN(c4ccc([C@@H]5c6ccc(O)cc6CC[C@@H]5c5ccccc5)cc4)CC3)OC2)CC1] | (S)-3-(1′-(((R)-9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-5-methoxy-6-oxo- 6,8-dihydro-2H,7H- spiro[furo[2,3-e]isoindole- 3,4′-piperidin]-7- yl)piperidine-2,6-dione |
| A236 | 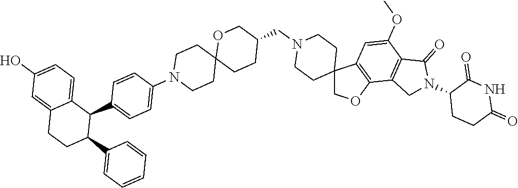 [SMILES: COc1cc2c(c3c1C(=O)N([C@H]1CCC(=O)NC1=O)C3)OCC21CCN(C[C@@H]2CCC3(CCN(c4ccc([C@@H]5c6ccc(O)cc6CC[C@@H]5c5ccccc5)cc4)CC3)OC2)CC1] | (S)-3-(1′-(((S)-9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-5-methoxy-6-oxo- 6,8-dihydro-2H,7H- spiro[furo[2,3-e]isoindole- 3,4′-piperidin]-7- yl)piperidine-2,6-dione |

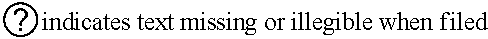

In certain embodiments, the compound is selected from the compounds in Tables 1-3, or a pharmaceutically acceptable salt thereof.

In certain embodiments, the compound is selected from the compounds in Tables 1-3.

In certain embodiments, the compound is selected from the compounds in Tables 1 and 2, or a pharmaceutically acceptable salt thereof.

In certain embodiments, the compound is selected from the compounds in Tables 1 and 2.

In certain embodiments, the compound is selected from the compounds in Table 1, or a pharmaceutically acceptable salt thereof.

In certain embodiments, the compound is selected from the compounds in Table 1.

In certain embodiments, the compound is selected from the compounds in Table 2, or a pharmaceutically acceptable salt thereof.

In certain embodiments, the compound is selected from the compounds in Table 2.

In certain embodiments, the compound is selected from the compounds in Table 3, or a pharmaceutically acceptable salt thereof.

In certain embodiments, the compound is selected from the compounds in Table 3.

TABLE 1
*designates that it is enantiomerically pure with respect to the warhead (tetrahydronaphthalene) but the absolute configuration is not
determined; the relative configuration of the two stereogenic centers is cis.

| Compound | | |
|---|---|---|
| No. | Structure | Compound Name |
| A1* | 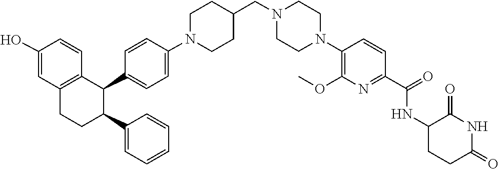 [SMILES: COc1nc(C(=O)NC2CCC(=O)NC2=O)ccc1N1CCN(CC2CCN(c3ccc([C@@H]4c5ccc(O)cc5CC[C@@H]4c4ccccc4)cc3)CC2)CC1] | N-(2,6-dioxopiperidin-3-yl)-5- (4-((1-(4-((1R,2S)-6-hydroxy- 2-phenyl-1,2,3,4-tetrahydro- naphthalen-1-yl)phenyl)piper- idin-4-yl)methyl)piperazin-1- yl)-6-methoxypicolinamide |
| A2 | 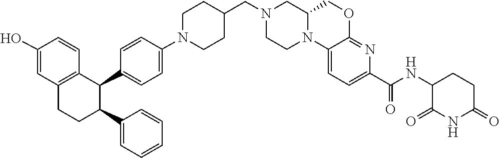 [SMILES: O=C1CCC(NC(=O)c2ccc3c(n2)OC[C@H]2CN(CC4CCN(c5ccc([C@@H]6c7ccc(O)cc7CC[C@@H]6c6ccccc6)cc5)CC4)CCN32)C(=O)N1] | (4aR)-N-(2,6-dioxopiperidin- 3-yl)-3-((1-(4-((1R,2S)-6- hydroxy-2-phenyl-1,2,3,4- tetrahydronaphthalen-1-yl)- phenyl)piperidin-4-yl)meth- yl)-1,2,3,4,4a,5-hexahydro- pyrazino[1,2-d]pyrido[2,3-b] [1,4]oxazine-8-carboxamide |
| A3 | 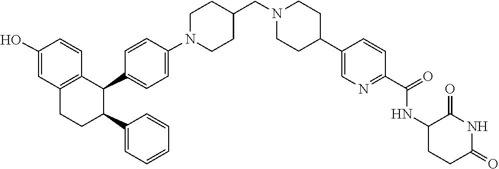 [SMILES: O=C1CCC(NC(=O)c2ccc(C3CCN(CC4CCN(c5ccc([C@@H]6c7ccc(O)cc7CC[C@@H]6c6ccccc6)cc5)CC4)CC3)cn2)C(=O)N1] | N-(2,6-dioxopiperidin-3-yl)-5- (1-((1-(4-((1R,2S)-6-hydroxy- 2-phenyl-1,2,3,4-tetrahydro- naphthalen-1-yl)phenyl)piper- idin-4-yl)methyl)piperidin-4- yl)picolinamide |
| A4 | 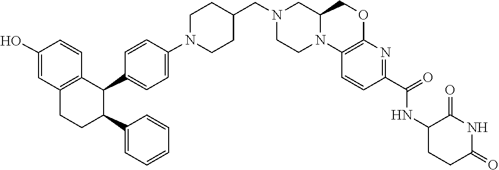 [SMILES: O=C1CCC(NC(=O)c2ccc3c(n2)OC[C@@H]2CN(CC4CCN(c5ccc([C@@H]6c7ccc(O)cc7CC[C@@H]6c6ccccc6)cc5)CC4)CCN32)C(=O)N1] | (4aR)-N-(2,6-dioxopiperidin- 3-yl)-3-((1-(4-((1S,2R)-6- hydroxy-2-phenyl-1,2,3,4- tetrahydronaphthalen-1-yl)- phenyl)piperidin-4-yl)meth- yl)-1,2,3,4,4a,5-hexahydro- pyrazino[1,2-d]pyrido[2,3-b] [1,4]oxazine-8-carboxamide |
| A5 | 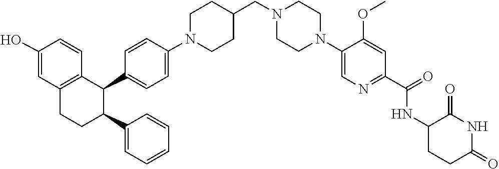 [SMILES: COc1cc(C(=O)NC2CCC(=O)NC2=O)ncc1N1CCN(CC2CCN(c3ccc([C@@H]4c5ccc(O)cc5CC[C@@H]4c4ccccc4)cc3)CC2)CC1] | N-(2,6-dioxopiperidin-3-yl)-5- (4-((1-(4-((1R,2S)-6-hydroxy- 2-phenyl-1,2,3,4-tetrahydro- naphthalen-1-yl)phenyl)piper- idin-4-yl)methyl)piperazin-1- yl)-4-methoxypicolinamide |
| A6* | 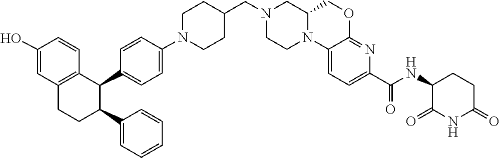 [SMILES: O=C1CC[C@H](NC(=O)c2ccc3c(n2)OC[C@H]2CN(CC4CCN(c5ccc([C@@H]6c7ccc(O)cc7CC[C@@H]6c6ccccc6)cc5)CC4)CCN32)C(=O)N1] | (4aR)-N-(2,6-dioxopiperidin- 3-yl)-3-((1-(4-((1R,2S)-6- hydroxy-2-phenyl-1,2,3,4- tetrahydronaphthalon-1-yl)- phenyl)piperidin-4-yl)meth- yl)-1,2,3,4,4a,5-hexahydro- pyrazino[1,2-d]pyrido[2,3-b] [1,4]oxazine-8-carboxamide |
| A7* | 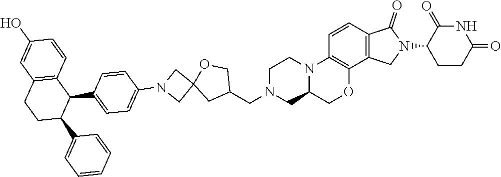 [SMILES: O=C1CC[C@H](N2Cc3c(ccc4c3OC[C@H]3CN(CC5COC6(C5)CN(c5ccc([C@@H]7c8ccc(O)cc8CC[C@@H]7c7ccccc7)cc5)C6)CCN43)C2=O)C(=O)N1] | (3S)-3-((5aR)-7-((2-(4-((1R, 2S)-6-hydroxy-2-phenyl- 1,2,3,4-tetrahydronaphthalen- 1-yl)phenyl)-5-oxa-2-azaspiro [3.4]octan-7-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A8* | 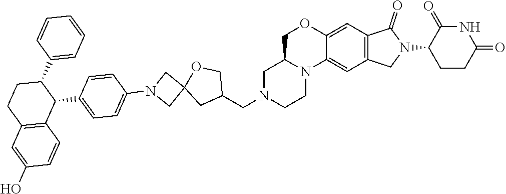 [SMILES: O=C1CC[C@H](N2Cc3cc4c(cc3C2=O)OC[C@@H]2CN(CC3COC5(C3)CN(c3ccc([C@@H]6c7ccc(O)cc7CC[C@@H]6c6ccccc6)cc3)C5)CCN42)C(=O)N1] | (S)-3-((S)-3-(((S)-2-(4-((1R, 2S)-6-hydroxy-2-phenyl- 1,2,3,4-tetrahydronaphthalen- 1-yl)phenyl)-5-oxa-2-azaspiro [3.4]oCtan-7-yl)methyl)-8- oxo-1,2,3,4,4a,5,8,10-octa- hydro-9H-pyrazino[1′,2′:4,5] [1,4]oxazino[2,3-f]isoindol- 9-yl)piperidine-2,6-dione |
| A9* | 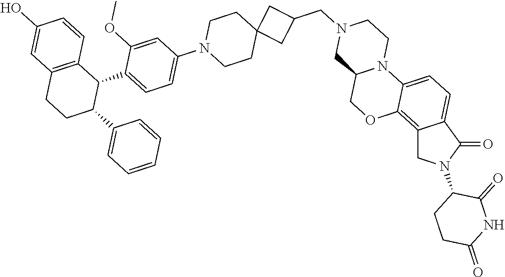 [SMILES: COc1cc(N2CCC3(CC2)CC(CN2CCN4c5ccc6c(c5OC[C@H]4C2)CN([C@H]2CCC(=O)NC2=O)C6=O)C3)ccc1[C@H]1c2ccc(O)cc2CC[C@H]1c1ccccc1] | (S)-3-((R)-7-((7-(4-((1R,2R)- 6-hydroxy-2-phenyl-1,2,3,4- tetrahydronaphthalen-1-yl)-3- methoxyphenyl)-7-azaspiro [3.5]nonan-2-yl)methyl)-1- oxo-1,3,5,5a,6,7,8,9-oCta- hydro-2H-pyrazino[1,2′:4,5] [1,4]oxazino[2,3-e]isoindol-2- yl)piperidine-2,6-dione |
| A10* | 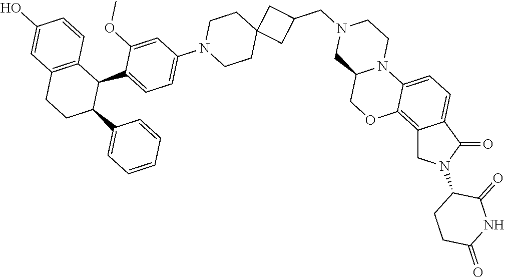 [SMILES: COc1cc(N2CCC3(CC2)CC(CN2CCN4c5ccc6c(c5OC[C@H]4C2)CN([C@H]2CCC(=O)NC2=O)C6=O)C3)ccc1[C@@H]1c2ccc(O)cc2CC[C@@H]1c1ccccc1] | (S)-3-((R)-7-((7-(4-((1S,2S)- 6-hydroxy-2-phenyl-1,2,3,4- tetrahydronaphthalen-1-yl)-3- methoxyphenyl)-7-azaspiro [3.5]nonan-2-yl)methyl)-1- oxo-1,3,5,5a,6,7,8,9-oCta- hydro-2H-pyrazino[1′,2′:4,5] [1,4]oxazino[2,3-e]isoindol-2- yl)piperidine-2,6-dione |
| A11* | 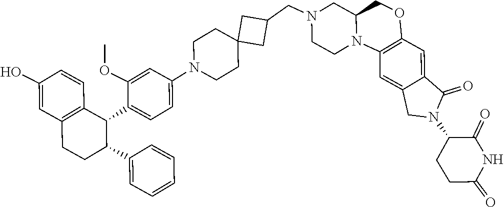 [SMILES: COc1cc(N2CCC3(CC2)CC(CN2CCN4c5cc6c(cc5OC[C@@H]4C2)C(=O)N([C@H]2CCC(=O)NC2=O)C6)C3)ccc1[C@H]1c2ccc(O)cc2CC[C@H]1c1ccccc1] | (S)-3-((S)-3-((7-(4-((1R,2R)- 6-hydroxy-2-phenyl-1,2,3,4- tetrahydronaphthalen-1-yl)-3- methoxyphenyl)-7-azaspiro [3.5]nonan-2-yl)methyl)-8- oxo-1,2,3,4,4a,5,8,10-oCta- hydro-9H-pyrazino[1′,2′:4,5] [1,4]oxazino[2,3-f]isoindol-9- yl)piperidine-2,6-dione |
| A12* | 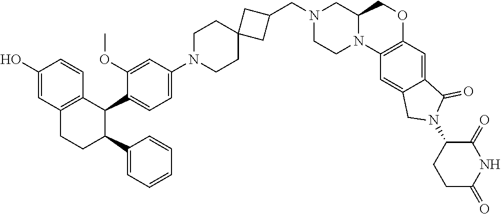 [SMILES: COc1cc(N2CCC3(CC2)CC(CN2CCN4c5cc6c(cc5OC[C@@H]4C2)C(=O)N([C@H]2CCC(=O)NC2=O)C6)C3)ccc1[C@@H]1c2ccc(O)cc2CC[C@@H]1c1ccccc1] | (S)-3-((S)-3-((7-(4-((1S,2S)-6- hydroxy-2-phenyl-1,2,3,4- tetrahydronaphthalen-1-yl)-3- methoxyphenyl)-7-azaspiro [3.5]nonan-2-yl)methyl)-8- oxo-1,2,3,4,4a,5,8,10-oCta- hydro-9H-pyrazino[1′,2′:4,5] [1,4]oxazino[2,3-f]isoindol-9- yl)piperidine-2,6-dione |
| A13* | 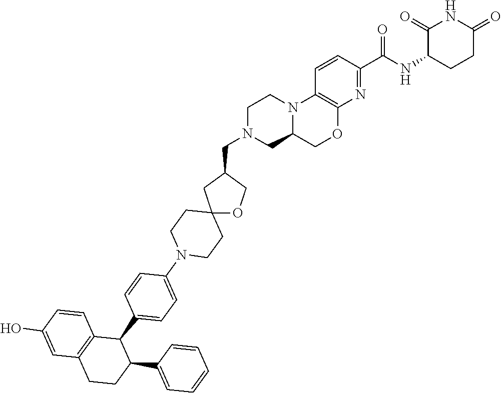 [SMILES: O=C1CC[C@H](NC(=O)c2ccc3c(n2)OC[C@H]2CN(C[C@H]4COC5(CCN(c6ccc([C@@H]7c8ccc(O)cc8CC[C@@H]7c7ccccc7)cc6)CC5)C4)CCN32)C(=O)N1] | (R)-N-((S)-2,6-dioxopiperidin- 3-yl)-3-(((S)-8-(4-((1R,2S)-6- hydroxy-2-phenyl-1,2,3,4- tetrahydronaphthalen-1-yl)- phenyl)-1-oxa-8-azaspiro[4.5] decan-3-yl)methyl)-1,2,3,4,4a, 5-hexahydropyrazino[1,2-d] pyrido[2,3-b][1,4]oxazine-8- carboxamide |
| A14* | 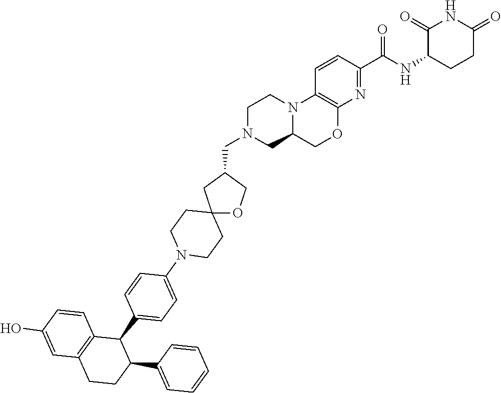 [SMILES: O=C1CC[C@H](NC(=O)c2ccc3c(n2)OC[C@H]2CN(C[C@@H]4COC5(CCN(c6ccc([C@@H]7c8ccc(O)cc8CC[C@@H]7c7ccccc7)cc6)CC5)C4)CCN32)C(=O)N1] | (R)-N-((S)-2,6-dioxopiperidin- 3-yl)-3-(((R)-8-(4-((1R,2S)- 6-hydroxy-2-phenyl-1,2,3,4- tetrahydronaphthalen-1-yl)- phenyl)-1-oxa-8-azaspiro[4.5] decan-3-yl)methyl)-1,2,3,4,4a, 5-hexahydropyrazino[1,2-d] pyrido[2,3-b][1,4]oxazine-8- carboxamide |
| A15* | 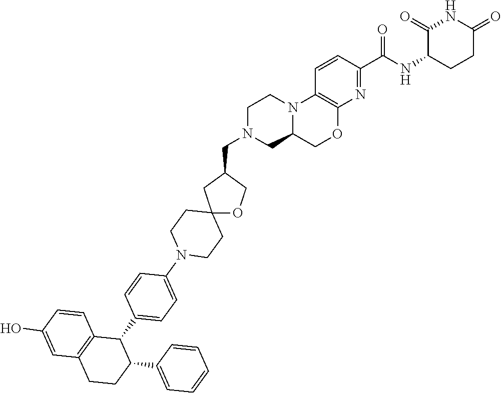 | (R)-N-((S)-2,6-dioxopiperidin- 3-yl)-3-(((S)-8-(4-((1S,2R)-6- hydroxy-2-phenyl-1,2,3,4- tetrahydronaphthalen-1-yl)- phenyl)-1-oxa-8-azaspiro[4.5] decan-3-yl)methyl)-1,2,3,4,4a, 5-hexahydropyrazino[1,2-d] pyrido[2,3-b][1,4]oxazine-8- carboxamide |
| A16* | 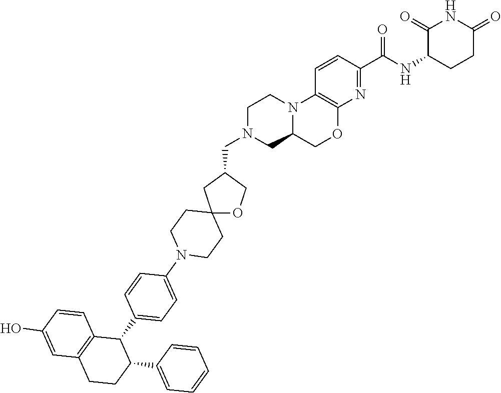 [SMILES: O=C1CC[C@H](NC(=O)c2ccc3c(n2)OC[C@H]2CN(C[C@@H]4COC5(CCN(c6ccc([C@H]7c8ccc(O)cc8CC[C@H]7c7ccccc7)cc6)CC5)C4)CCN32)C(=O)N1] | (R)-N-((S)-2,6-dioxopiperidin- 3-yl)-3-(((R)-8-(4-((1S,2R)- 6-hydroxy-2-phenyl-1,2,3,4- tetrahydronaphthalen-1-yl)- phenyl)-1-oxa-8-azaspiro[4.5] decan-3-yl)methyl)-1,2,3,4,4a, 5-hexahydropyrazino[1,2-d] pyrido[2,3-b][1,4]oxazine-8- carboxamide |
| A17* | 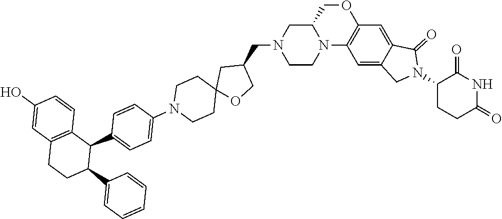 [SMILES: O=C1CC[C@H](N2Cc3cc4c(cc3C2=O)OC[C@H]2CN(C[C@H]3COC5(CCN(c6ccc([C@@H]7c8ccc(O)cc8CC[C@@H]7c7ccccc7)cc6)CC5)C3)CCN42)C(=O)N1] | (S)-3-((R)-3-(((S)-8-(4-((1R, 2S)-6-hydroxy-2-phenyl- 1,2,3,4-tetrahydronaphthalen- 1-yl)phenyl)-1-oxa-8-azaspiro [4.5]decan-3-yl)methyl)-8-oxo- 1,2,3,4,4a,5,8,10-oCtahydro- 9H-yrazino[1′,2′:4,5][1,4] oxazino[2,3-f]isoindol-9-yl)- piperidine-2,6-dione |
| A18* | 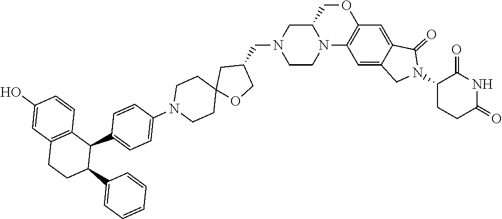 [SMILES: O=C1CC[C@H](N2Cc3cc4c(cc3C2=O)OC[C@H]2CN(C[C@@H]3COC5(CCN(c6ccc([C@@H]7c8ccc(O)cc8CC[C@@H]7c7ccccc7)cc6)CC5)C3)CCN42)C(=O)N1] | (S)-3-((R)-3-(((R)-8-(4-((1R, 2S)-6-hydroxy-2-phenyl- 1,2,3,4-tetrahydronaphthalen- 1-yl)phenyl)-1-oxa-8-azaspiro [4.5]decan-3-yl)methyl)-8-oxo- 1,2,3,4,4a,5,8,10-oCtahydro- 9H-yrazino[1′,2′:4,5][1,4] oxazino[2,3-f]isoindol-9-yl)- piperidine-2,6-dione |
| A19* | 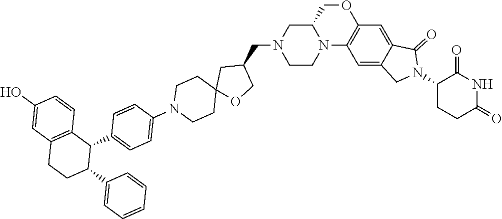 [SMILES: O=C1CC[C@H](N2Cc3cc4c(cc3C2=O)OC[C@H]2CN(C[C@H]3COC5(CCN(c6ccc([C@H]7c8ccc(O)cc8CC[C@H]7c7ccccc7)cc6)CC5)C3)CCN42)C(=O)N1] | (S)-3-((R)-3-(((S)-8-(4-((1S, 2R)-6-hydroxy-2-phenyl- 1,2,3,4-tetrahydronaphthalen- 1-yl)phenyl)-1-oxa-8-azaspiro [4.5]decan-3-yl)methyl)-8-oxo- 1,2,3,4,4a,5,8,10-oCtahydro- 9H-pyrazino[1′,2′:4,5][1,4] oxazino[2,3-f]isoindol-9-yl)- piperidine-2,6-dione |
| A20* | 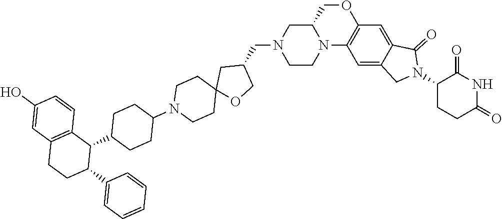 [SMILES: O=C1CC[C@H](N2Cc3cc4c(cc3C2=O)OC[C@H]2CN(C[C@@H]3COC5(CCN(C6CCC([C@H]7c8ccc(O)cc8CC[C@H]7c7ccccc7)CC6)CC5)C3)CCN42)C(=O)N1] | (S)-3-((R)-3-(((R)-8-(4-((1S, 2R)-6-hydroxy-2-phenyl- 1,2,3,4-tetrahydronaphthalen- 1-yl)phenyl)-1-oxa-8-azaspiro [4.5]decan-3-yl)methyl)-8-oxo- 1,2,3,4,4a,5,8,10-oCtahydro- 9H-pyrazino[1′,2′:4,5][1,4] oxazino[2,3-f]isoindol-9-yl)- piperidine-2,6-dione |
| A21* | 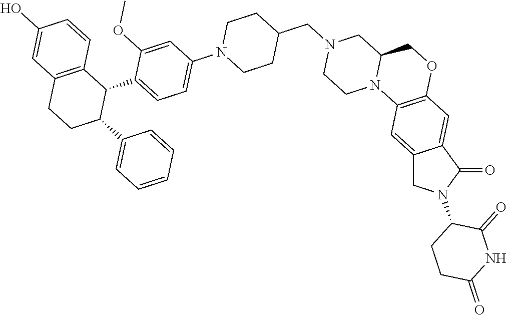 [SMILES: COc1cc(N2CCC(CN3CCN4c5cc6c(cc5OC[C@@H]4C3)C(=O)N([C@H]3CCC(=O)NC3=O)C6)CC2)ccc1[C@H]1c2ccc(O)cc2CC[C@H]1c1ccccc1] | (S)-3-((S)-3-((1-(4-((1R,2R)- 6-hydroxy-2-phenyl-1,2,3,4- tetrahydronaphthalen-1-yl)-3- methoxyphenyl)piperidin-4- yl)methyl)-8-oxo-1,2,3,4,4a, 5,8,10-oCtahydro-9H-pyrazino [1′,2′:4,5][1,4]oxazino[2,3-f] isoindol-9-yl)piperidine-2,6- dione |
| A22* | 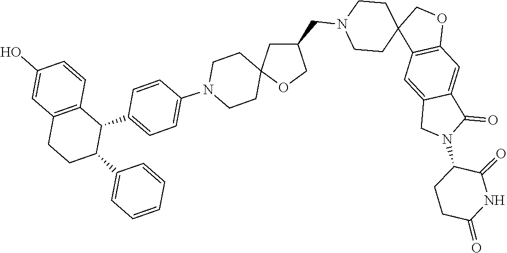 [SMILES: O=C1CC[C@H](N2Cc3cc4c(cc3C2=O)OCC42CCN(C[C@H]3COC4(CCN(c5ccc([C@H]6c7ccc(O)cc7CC[C@H]6c6ccccc6)cc5)CC4)C3)CC2)C(=O)N1] | (S)-3-(1′-(((S)-8-(4-((1S,2R)- 6-hydroxy-2-phenyl-1,2,3,4- tetrahydronaphthalen-1-yl)- phenyl)-1-oxa-8-azaspiro[4.5] decan-3-yl)methyl)-7-oxo-5,7- dihydro-2H,6H-spiro[furo[2,3- f]isoindolc-3,4′-pipcridin]-6- yl)pipcridinc-2,6-dione |
| A23* | 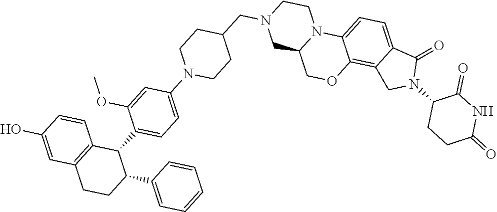 [SMILES: COc1cc(N2CCC(CN3CCN4c5ccc6c(c5OC[C@H]4C3)CN([C@H]3CCC(=O)NC3=O)C6=O)CC2)ccc1[C@H]1c2ccc(O)cc2CC[C@H]1c1ccccc1] | (R)-3-((S)-7-((1-(4-((1S,2S)- 6-hydroxy-2-phenyl-1,2,3,4- tetrahydronaphthalen-1-yl)-3- methoxyphenyl)piperidin-4- yl)methyl)-1-oxo-1,3,5,5a,6,7, 8,9-oCtahydro-2H-pyrazino [1′,2′:4,5][1,4]oxazino[2,3-e] isoindol-2-yl)piperidine-2,6- dione |
| A24* | 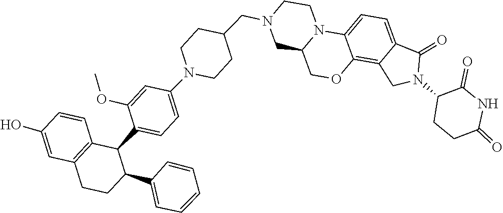 [SMILES: COc1cc(N2CCC(CN3CCN4c5ccc6c(c5OC[C@H]4C3)CN([C@H]3CCC(=O)NC3=O)C6=O)CC2)ccc1[C@@H]1c2ccc(O)cc2CC[C@@H]1c1ccccc1] | (R)-3-((S)-7-((1-(4-((1R,2R)- 6-hydroxy-2-phenyl-1,2,3,4- tetrahydronaphthalen-1-yl)-3- methoxyphenyl)piperidin-4- yl)methyl)-1-oxo-1,3,5,5a,6,7, 8,9-oCtahydro-2H-pyrazino [1′,2′:4,5][1,4]oxazino[2,3-e] isoindol-2-yl)piperidine-2,6- dione |
| A25* | 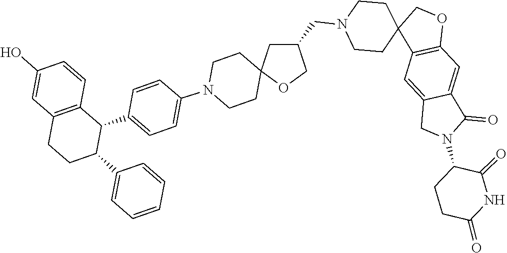 [SMILES: O=C1CC[C@H](N2Cc3cc4c(cc3C2=O)OCC42CCN(C[C@@H]3COC4(CCN(c5ccc([C@H]6c7ccc(O)cc7CC[C@H]6c6ccccc6)cc5)CC4)C3)CC2)C(=O)N1] | (3S)-3-((5aR)-7-((2-(4-((1S, 2R)-6-hydroxy-2-phenyl- 1,2,3,4-tetrahydronaphthalen- 1-yl)phenyl)-5-oxa-2-azaspiro [3.4]octan-7-yl)methyl)-1-oxo- 1,3,5,5a,6,7,8,9-octahydro-2H- pyrazino[1′,2′:4,5][1,4]oxazino [2,3-c]isoindol-2-yl)piperidine- 2,6-dione |
| A26* | 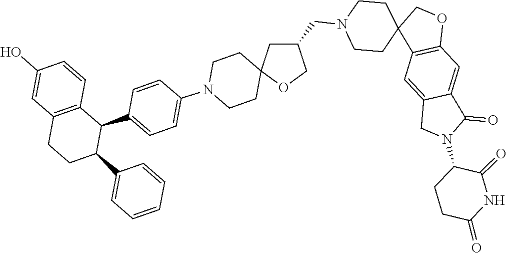 [SMILES: O=C1CC[C@H](N2Cc3cc4c(cc3C2=O)OCC42CCN(C[C@@H]3COC4(CCN(c5ccc([C@@H]6c7ccc(O)cc7CC[C@@H]6c6ccccc6)cc5)CC4)C3)CC2)C(=O)N1] | (3S)-3-(1′-((8-(4-((1R,2S)-6- hydroxy-2-phenyl-1,2,3,4- tetrahydronaphthalen-1-yl)- phenyl)-1-oxa-8-azaspiro[4.5] decan-3-yl)methyl)-7-oxo-5,7- dihydro-2H,6H-spiro[furo[2,3- f]isoindole-3,4′-piperidin]-6- yl)piperidine-2,6-dione |
| A27* | 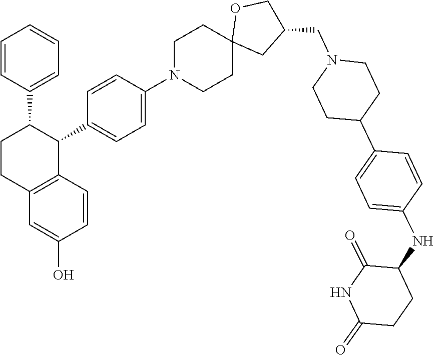 [SMILES: O=C1CC[C@H](Nc2ccc(C3CCN(C[C@H]4COC5(CCN(c6ccc([C@@H]7c8ccc(O)cc8CC[C@@H]7c7ccccc7)cc6)CC5)C4)CC3)cc2)C(=O)N1] | (R)-3-((4-(1-(((R)-8-(4-((1S, 2R)-6-hydroxy-2-phenyl- 1,2,3,4-tetrahydronaphthalen- 1-yl)phenyl)-1-oxa-8-azaspiro [4.5]decan-3-yl)methyl)piper- idin-4-yl)phenyl)amino)piper- idine-2,6-dione |
| A28* | 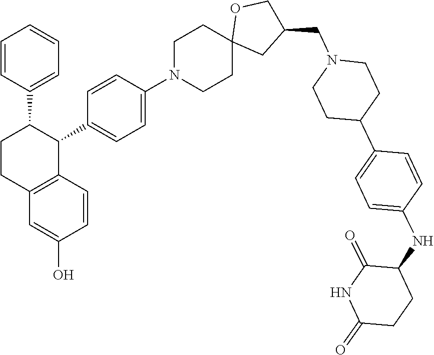 [SMILES: O=C1CC[C@H](Nc2ccc(C3CCN(C[C@@H]4COC5(CCN(c6ccc([C@@H]7c8ccc(O)cc8CC[C@@H]7c7ccccc7)cc6)CC5)C4)CC3)cc2)C(=O)N1] | (R)-3-((4-(1-(((S)-8-(4-(1S, 2R)-6-hydroxy-2-phenyl- 1,2,3,4-tetrahydronaphthalen- 1-yl)phenyl)-1-oxa-8-azaspiro [4.5]decan-3-yl)methyl)piper- idin-4-yl)phenyl)amino)piper- idine-2,6-dione |
| A29* | 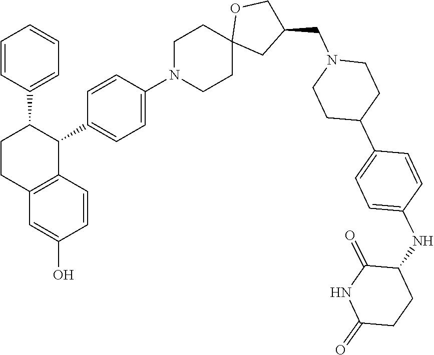 [SMILES: O=C1CC[C@@H](Nc2ccc(C3CCN(C[C@@H]4COC5(CCN(c6ccc([C@@H]7c8ccc(O)cc8CC[C@@H]7c7ccccc7)cc6)CC5)C4)CC3)cc2)C(=O)N1] | (R)-3-((4-(1-(((R)-8-(4-((1R, 2S)-6-hydroxy-2-phenyl- 1,2,3,4-tetrahydronaphthalen- 1-yl)phenyl)-1-oxa-8-azaspiro [4.5]decan-3-yl)methyl)piper- idin-4-yl)phenyl)amino)piper- idine-2,6-dione |
| A30* | 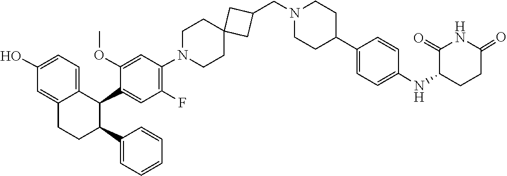 [SMILES: COc1cc(N2CCC3(CC2)CC(CN2CCC(c4ccc(N[C@H]5CCC(=O)NC5=O)cc4)CC2)C3)c(F)cc1[C@@H]1c2ccc(O)cc2CC[C@@H]1c1ccccc1] | (S)-3-((4-(1-((7-(2-fluoro-4- ((1S,2S)-6-hydroxy-2-phenyl- 1,2,3,4-tetrahydronaphthalen- 1-yl)-5-methoxyphenyl)-7-aza- spiro[3.5]nonan-2-yl)methyl)- piperidin-4-yl)phenyl)amino)- piperidine-2,6-dione |
| A31* | 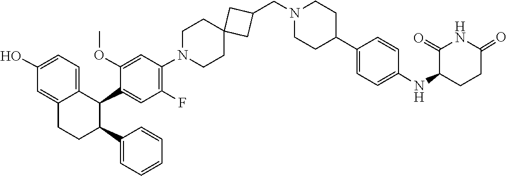 [SMILES: COc1cc(N2CCC3(CC2)CC(CN2CCC(c4ccc(N[C@@H]5CCC(=O)NC5=O)cc4)CC2)C3)c(F)cc1[C@@H]1c2ccc(O)cc2CC[C@@H]1c1ccccc1] | (R)-3-((4-(1-((7-(2-fluoro-4- ((1S,2S)-6-hydroxy-2-phenyl- 1,2,3,4-tetrahydronaphthalen- 1-yl)-5-methoxyphenyl)-7- azaspiro[3.5]nonan-2-yl)meth- yl)piperidin-4-yl)phenyl)- amino)piperidine-2,6-dione |
| A32* | 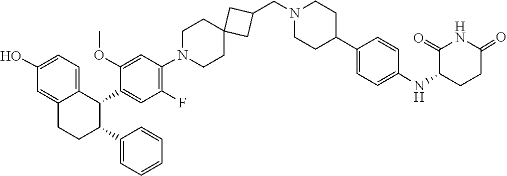 [SMILES: COc1cc(N2CCC3(CC2)CC(CN2CCC(c4ccc(N[C@H]5CCC(=O)NC5=O)cc4)CC2)C3)c(F)cc1[C@H]1c2ccc(O)cc2CC[C@H]1c1ccccc1] | (S)-3-((4-(1-((7-(2-fluoro-4- ((1R,2R)-6-hydroxy-2-phenyl- 1,2,3,4-tetrahydronaphthalen- 1-yl)-5-methoxyphenyl)-7- azaspiro[3.5]nonan-2-yl)meth- yl)piperidin-4-yl)phenyl)- amino)-piperidine-2,6-dione |
| A33* | 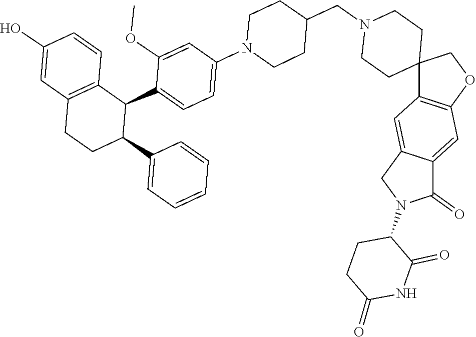 [SMILES: COc1cc(N2CCC(CN3CCC4(CC3)COc3cc5c(cc34)CN([C@H]3CCC(=O)NC3=O)C5=O)CC2)ccc1[C@@H]1c2ccc(O)cc2CC[C@@H]1c1ccccc1] | (R)-3-(1′-((1-(4-((1R,2R)-6- hydroxy-2-phenyl-1,2,3,4- tetrahydronaphthalen-1-yl)-3- methoxyphenyl)piperidin-4- yl)methyl)-7-oxo-5,7-dihydro- 2H,6H-spiro[furo[2,3-f]iso- indole-3,4′-piperidin]-6-yl)- piperidine-2,6-dione |
| A34* | 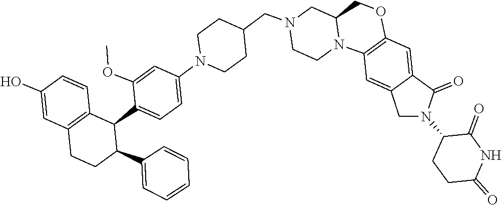 [SMILES: COc1cc(N2CCC(CN3CCN4c5cc6c(cc5OC[C@@H]4C3)C(=O)N([C@H]3CCC(=O)NC3=O)C6)CC2)ccc1[C@@H]1c2ccc(O)cc2CC[C@@H]1c1ccccc1] | (S)-3-((S)-3-((1-(4-((1S,2S)-6- hydroxy-2-phenyl-1,2,3,4- tetrahydronaphthalen-1-yl)-3- methoxyphenyl)piperidin-4- yl)methyl)-8-oxo-1,2,3,4,4a,5, 8,10-oCtahydro-9H-pyrazino [1′,2′:4,5][1,4]oxazino[2,3-f] isoindol-9-yl)piperidine-2,6- dione |
| A35 | 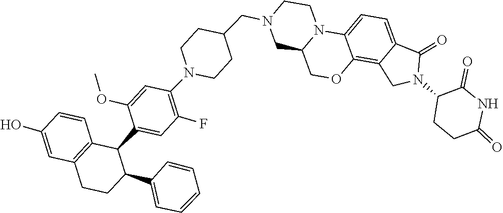 [SMILES: COc1cc(N2CCC(CN3CCN4c5ccc6c(c5OC[C@H]4C3)CN([C@H]3CCC(=O)NC3=O)C6=O)CC2)c(F)cc1[C@@H]1c2ccc(O)cc2CC[C@@H]1c1ccccc1] | (S)-3-((R)-7-((1-(2-fluoro-4- ((1S,2S)-6-hydroxy-2-phenyl- 1,2,3,4-tetrahydronaphthalen- 1-yl)-5-methoxyphenyl)piper- idin-4-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A36* | 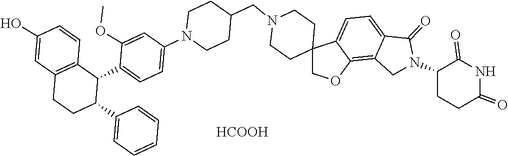 [SMILES: COc1cc(N2CCC(CN3CCC4(CC3)COc3c4ccc4c3CN([C@H]3CCC(=O)NC3=O)C4=O)CC2)ccc1[C@H]1c2ccc(O)cc2CC[C@H]1c1ccccc1 ; "HCOOH"] | (R)-3-(1′-((1-(4-((1S,2S)-6- hydroxy-2-phenyl-1,2,3,4- tetrahydronaphthalen-1-yl)-3- methoxyphenyl)piperidin-4- yl)methyl)-6-oxo-6,8-dihydro- 2H,7H-spiro[furo[2,3-e]iso- indole-3,4′-piperidin]-7-yl)- piperidine-2,6-dione formate |
| A37* | 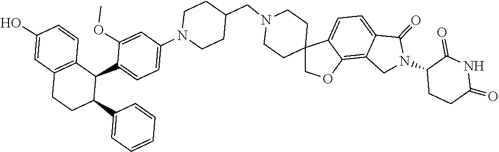 [SMILES: COc1cc(N2CCC(CN3CCC4(CC3)COc3c4ccc4c3CN([C@H]3CCC(=O)NC3=O)C4=O)CC2)ccc1[C@@H]1c2ccc(O)cc2CC[C@@H]1c1ccccc1] | (R)-3-(1′-((1-(4-((1R,2R)-6- hydroxy-2-phenyl-1,2,3,4- tetrahydronaphthalen-1-yl)-3- methoxyphenyl)piperidin-4- yl)methyl)-6-oxo-6,8-dihydro- 2H,7H-spiro[furo[2,3-e]iso- indole-3,4′-piperidin]-7-yl)- piperidine-2,6-dione |
| A38* | 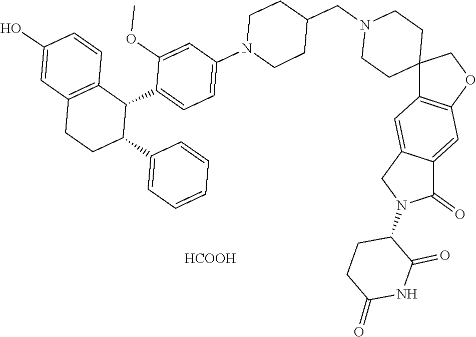 [SMILES: COc1cc(N2CCC(CN3CCC4(CC3)COc3cc5c(cc34)CN([C@H]3CCC(=O)NC3=O)C5=O)CC2)ccc1[C@H]1c2ccc(O)cc2CC[C@H]1c1ccccc1 ; "HCOOH"] | (R)-3-(1′-((1-(4-((1S,2S)-6- hydroxy-2-phenyl-1,2,3,4- tetrahydronaphthalen-1-yl)-3- methoxyphenyl)piperidin-4- yl)methyl)-7-oxo-5,7-dihydro- 2H,6H-spiro[furo[2,3-f]iso- indole-3,4′-piperidin]-6-yl)- piperidine-2,6-dione formate |
| A39 | 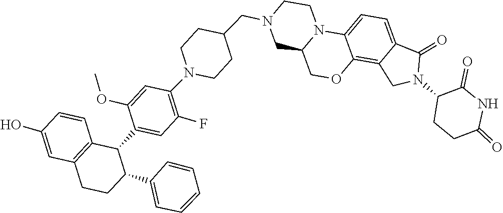 [SMILES: COc1cc(N2CCC(CN3CCN4c5ccc6c(c5OC[C@H]4C3)CN([C@H]3CCC(=O)NC3=O)C6=O)CC2)c(F)cc1[C@H]1c2ccc(O)cc2CC[C@H]1c1ccccc1] | (S)-3-((R)-7-((1-(2-fluoro-4- ((1R,2R)-6-hydroxy-2-phenyl- 1,2,3,4-tetrahydronaphthalen- 1-yl)-5-methoxyphenyl)piper- idin-4-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A40* | 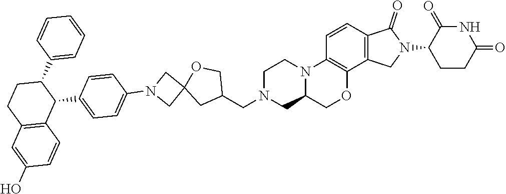 [SMILES: O=C1CC[C@H](N2Cc3c(ccc4c3OC[C@H]3CN(CC5COC6(C5)CN(c5ccc([C@@H]7c8ccc(O)cc8CC[C@@H]7c7ccccc7)cc5)C6)CCN43)C2=O)C(=O)N1] | (3S)-3-((5aR)-7-((2-(4-((1R, 2S)-6-hydroxy-2-phenyl- 1,2,3,4-tetrahydronaphthalen- 1-yl)phenyl)-5-oxa-2-azaspiro [3.4]octan-7-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A41* | 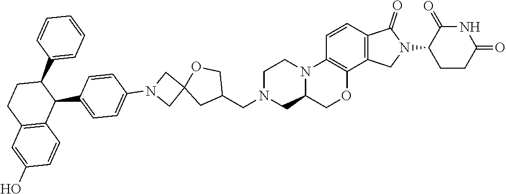 [SMILES: O=C1CC[C@H](N2Cc3c(ccc4c3OC[C@H]3CN(CC5COC6(C5)CN(c5ccc([C@H]7c8ccc(O)cc8CC[C@H]7c7ccccc7)cc5)C6)CCN43)C2=O)C(=O)N1] | (3S)-3-((5aR)-7-((2-(4-((1S, 2R)-6-hydroxy-2-phenyl- 1,2,3,4-tetrahydronaphthalen- 1-yl)phenyl)-5-oxa-2-azaspiro [3.4]octan-7-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A42* | 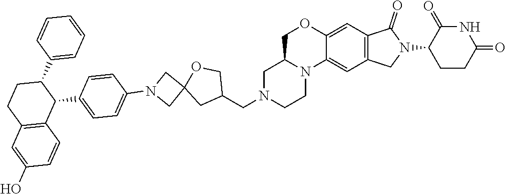 [SMILES: O=C1CC[C@H](N2Cc3cc4c(cc3C2=O)OC[C@@H]2CN(CC3COC5(C3)CN(c3ccc([C@@H]6c7ccc(O)cc7CC[C@@H]6c6ccccc6)cc3)C5)CCN42)C(=O)N1] | (3S)-3-((5aR)-7-((2-(4-((1R, 2R)-6-hydroxy-2-phenyl- 1,2,3,4-tetrahydronaphthalen- 1-yl)-3-methoxyphenyl)-5-oxa- 2-azaspiro[3.4]octan-7-yl)- methyl)-1-oxo-1,3,5,5a,6,7,8,9- octahydro-2H-pyrazino[1′,2′: 4,5][1,4]oxazino[2,3-e]isoindol- 2-yl)piperidine-2,6-dione |
| A43* | 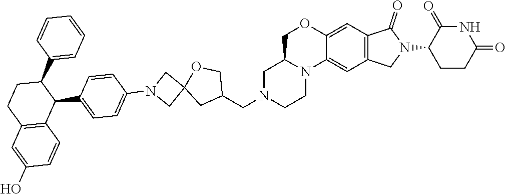 [SMILES: O=C1CC[C@H](N2Cc3cc4c(cc3C2=O)OC[C@@H]2CN(CC3COC5(C3)CN(c3ccc([C@H]6c7ccc(O)cc7CC[C@H]6c6ccccc6)cc3)C5)CCN42)C(=O)N1] | (3S)-3-((4aS)-3-((2-(4-((1S, 2R)-6-hydroxy-2-phenyl- 1,2,3,4-tetrahydronaphthalen- 1-yl)phenyl)-5-oxa-2-azaspiro [3.4]octan-7-yl)methyl)-8-oxo- 1,2,3,4,4a,5,8,10-octahydro- 9H-pyrazino[1′,2′:4,5][1,4] oxazino[2,3-f]isoindol-9-yl)- piperidine-2,6-dione |
| A44* | 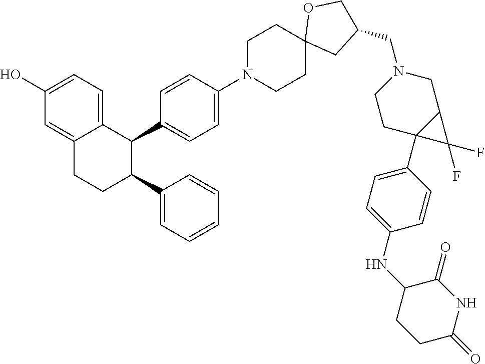 [SMILES: O=C1CCC(Nc2ccc(C34CCN(C[C@H]5COC6(CCN(c7ccc([C@@H]8c9ccc(O)cc9CC[C@@H]8c8ccccc8)cc7)CC6)C5)CC3C4(F)F)cc2)C(=O)N1] | (3S)-3-((4-(7,7-difluoro-3-(((S)- 8-(4-((1R,2S)-6-hydroxy-2- phenyl-1,2,3,4-tetrahydro- naphthalen-1-yl)phenyl)-1- oxa-8-azaspiro[4.5]decan-3-yl)- methyl)-3-azabicyclo[4.1.0] heptan-6-yl)phenyl)amino)- piperidine-2,6-dione |
| A45* | 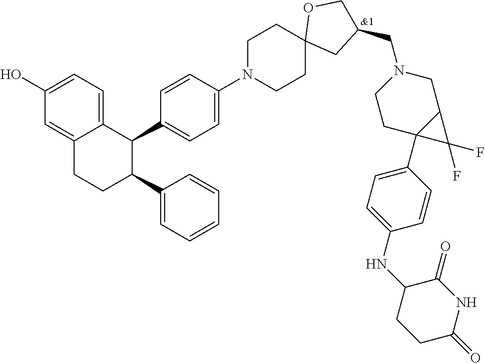 [SMILES: O=C1CCC(Nc2ccc(C34CCN(C[C@@H]5COC6(CCN(c7ccc([C@@H]8c9ccc(O)cc9CC[C@@H]8c8ccccc8)cc7)CC6)C5)CC3C4(F)F)cc2)C(=O)N1] | (3S)-3-((4-(7,7-difluoro-3- (((R)-8-(4-((1R,2S)-6-hydroxy- 2-phenyl-1,2,3,4-tetrahydro- naphthalen-1-yl)phenyl)-1-oxa- 8-azaspiro[4.5]decan-3-yl)- methyl)-3-azabicyclo[4.1.0] heptan-6-yl)phenyl)amino)- piperidine-2,6-dione |
| A46* | 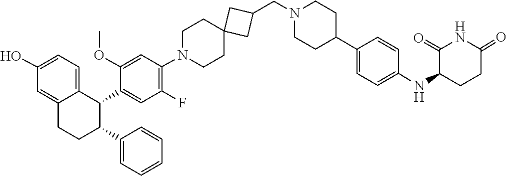 [SMILES: COc1cc(N2CCC3(CC2)CC(CN2CCC(c4ccc(N[C@@H]5CCC(=O)NC5=O)cc4)CC2)C3)c(F)cc1[C@H]1c2ccc(O)cc2CC[C@H]1c1ccccc1] | (R)-3-((4-(1-((7-(2-fluoro-4- ((1R,2R)-6-hydroxy-2-phenyl- 1,2,3,4-tetrahydronaphthalen- 1-yl)-5-methoxyphenyl)-7- azaspiro[3.5]nonan-2-yl)meth- yl)piperidin-4-yl)phenyl)- amino)piperidine-2,6-dione |
| A47* | 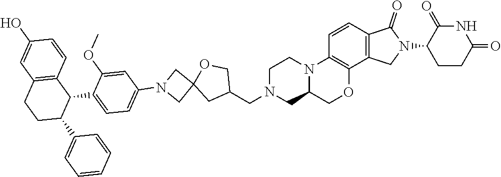 [SMILES: COc1cc(N2CC3(CC(CN4CCN5c6ccc7c(c6OC[C@H]5C4)CN([C@H]4CCC(=O)NC4=O)C7=O)CO3)C2)ccc1[C@H]1c2ccc(O)cc2CC[C@H]1c1ccccc1] | (3S)-3-((5aR)-7-((2-(4-((1R, 2R)-6-hydroxy-2-phenyl- 1,2,3,4-tetrahydronaphthalen- 1-yl)-3-methoxyphenyl)-5-oxa- 2-azaspiro[3.4]octan-7-yl)- methyl)-1-oxo-1,3,5,5a,6,7,8, 9-octahydro-2H-pyrazino [1′,2′:4,5][1,4]oxazino[2,3-e] isoindol-2-yl)piperidine-2,6- dione |
| A48* | 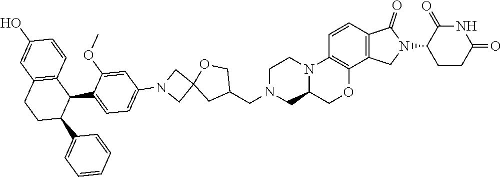 [SMILES: COc1cc(N2CC3(CC(CN4CCN5c6ccc7c(c6OC[C@H]5C4)CN([C@H]4CCC(=O)NC4=O)C7=O)CO3)C2)ccc1[C@@H]1c2ccc(O)cc2CC[C@@H]1c1ccccc1] | (3S)-3-((5aR)-7-((2-(4-((1S, 2S)-6-hydroxy-2-phenyl- 1,2,3,4-tetrahydronaphthalen- 1-yl)-3-methoxyphenyl)-5-oxa- 2-azaspiro[3.4]octan-7-yl)- methyl)-1-oxo-1,3,5,5a,6,7,8, 9-octahydro-2H-pyrazino [1′,2′:4,5][1,4]oxazino[2,3-e] isoindol-2-yl)piperidine-2,6- dione |
| A49* | 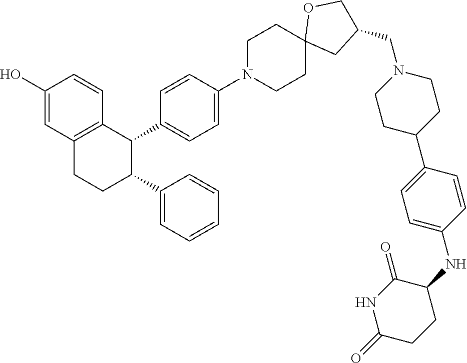 [SMILES: O=C1CC[C@H](Nc2ccc(C3CCN(C[C@H]4COC5(CCN(c6ccc([C@H]7c8ccc(O)cc8CC[C@H]7c7ccccc7)cc6)CC5)C4)CC3)cc2)C(=O)N1] | (S)-3-((4-(1-(((S)-8-(4-((1S, 2R)-6-hydroxy-2-phenyl- 1,2,3,4-tetrahydronaphthalen- 1-yl)phenyl)-1-oxa-8-azaspiro [4.5]dccan-3-yl)methyl)piper- idin-4-yl)phenyl)amino)piper- idine-2,6-dione |
| A50* | 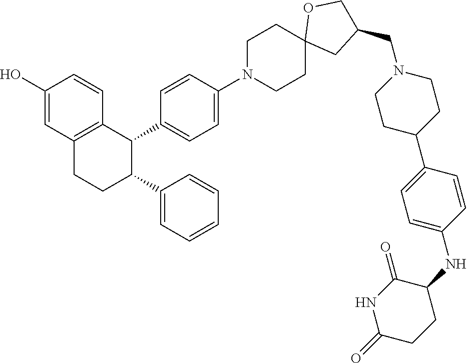 [SMILES: O=C1CC[C@H](Nc2ccc(C3CCN(C[C@@H]4COC5(CCN(c6ccc([C@H]7c8ccc(O)cc8CC[C@H]7c7ccccc7)cc6)CC5)C4)CC3)cc2)C(=O)N1] | (S)-3-((4-(1-(((R)-8-(4-((1S, 2R)-6-hydroxy-2-phenyl- 1,2,3,4-tetrahydronaphthalen- 1-yl)phenyl)-1-oxa-8-azaspiro [4.5]decan-3-yl)methyl)piper- idin-4-yl)phenyl)amino)piper- idine-2,6-dione |
| A51* | 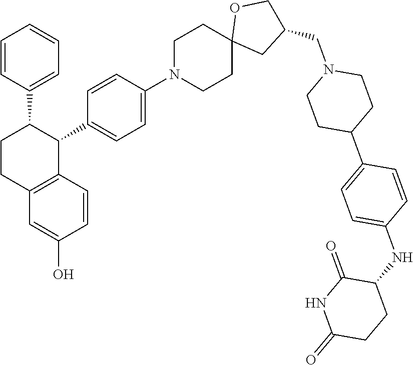 [SMILES: O=C1CC[C@@H](Nc2ccc(C3CCN(C[C@H]4COC5(CCN(c6ccc([C@@H]7c8ccc(O)cc8CC[C@@H]7c7ccccc7)cc6)CC5)C4)CC3)cc2)C(=O)N1] | (R)-3-((4-(1-(((S)-8-(4-((1R, 2S)-6-hydroxy-2-phenyl- 1,2,3,4-tetrahydronaphthalen- 1-yl)phenyl)-1-oxa-8-azaspiro [4.5]decan-3-yl)methyl)piper- idin-4-yl)phenyl)amino)piper- idine-2,6-dione |
| A52* | 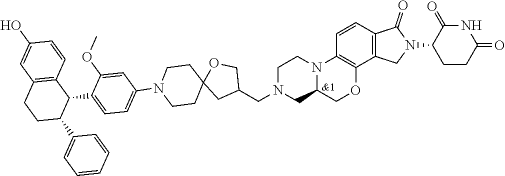 [SMILES: COc1cc(N2CCC3(CC2)CC(CN2CCN4c5ccc6c(c5OC[C@H]4C2)CN([C@H]2CCC(=O)NC2=O)C6=O)CO3)ccc1[C@H]1c2ccc(O)cc2CC[C@H]1c1ccccc1] | (3S)-3-((5aRS)-7-((8-(4-((1R, 2R)-6-hydroxy-2-phenyl- 1,2,3,4-tetrahydronaphthalen- 1-yl)-3-methoxyphenyl)-1- oxa-8-azaspiro[4.5]decan-3- yl)methyl)-1-oxo-1,3,5,5a,6,7, 8,9-octahydro-2H-pyrazino [1′,2′:4,5][1,4]oxazino[2,3-e] isoindol-2-yl)piperidine-2,6- dione |
| A53* | 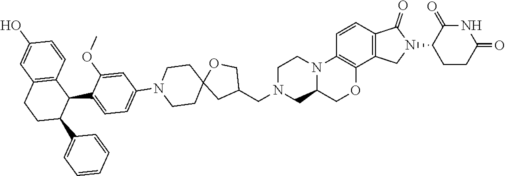 [SMILES: COc1cc(N2CCC3(CC2)CC(CN2CCN4c5ccc6c(c5OC[C@H]4C2)CN([C@H]2CCC(=O)NC2=O)C6=O)CO3)ccc1[C@@H]1c2ccc(O)cc2CC[C@@H]1c1ccccc1] | (3S)-3-((5aR)-7-((8-(4-((1S, 2S)-6-hydroxy-2-phenyl- 1,2,3,4-tetrahydronaphthalen- 1-yl)-3-methoxyphenyl)-1-oxa- 8-azaspiro[4.5]decan-3-yl)- methyl)-1-oxo-1,3,5,5a,6,7,8, 9-octahydro-2H-pyrazino[1′,2′: 4,5][1,4]oxazino[2,3-e]isoindol- 2-yl)piperidine-2,6-dione |
| A54* | 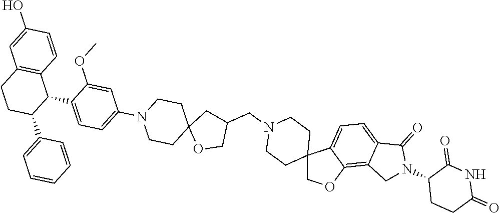 [SMILES: COc1cc(N2CCC3(CC2)CC(CN2CCC4(CC2)COc2c4ccc4c2CN([C@H]2CCC(=O)NC2=O)C4=O)CO3)ccc1[C@H]1c2ccc(O)cc2CC[C@H]1c1ccccc1] | (3S)-3-(1′-((8-(4-((1R,2R)-6- hydroxy-2-phenyl-1,2,3,4- tetrahydronaphthalen-1-yl)-3- methoxyphenyl)-1-oxa-8-aza- spiro[4.5]decan-3-yl)methyl)- 6-oxo-6,8-dihydro-2H,7H- spiro[furo[2,3-e]isoindole-3,4′- piperidin]-7-yl)piperidine-2,6- dione |
| A55* | 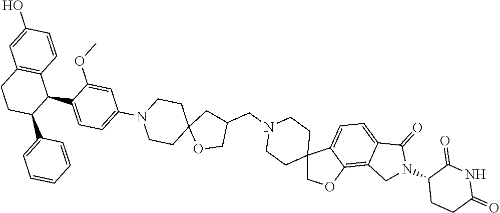 [SMILES: COc1cc(N2CCC3(CC2)CC(CN2CCC4(CC2)COc2c4ccc4c2CN([C@H]2CCC(=O)NC2=O)C4=O)CO3)ccc1[C@@H]1c2ccc(O)cc2CC[C@@H]1c1ccccc1] | (3S)-3-(1′-((8-(4-((1S,2S)-6- hydroxy-2-phenyl-1,2,3,4- tetrahydronaphthalen-1-yl)-3- methoxyphenyl)-1-oxa-8- azaspiro[4.5]decan-3-yl)meth- yl)-6-oxo-6,8-dihydro-2H,7H- spiro[furo[2,3-e]isoindole-3,4′- piperidin]-7-yl)piperidine-2,6- dione |
| A56* | 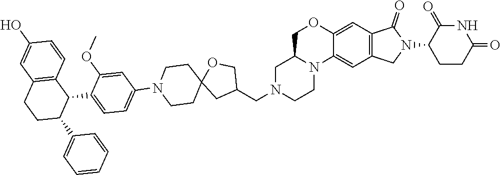 [SMILES: COc1cc(N2CCC3(CC2)CC(CN2CCN4c5cc6c(cc5OC[C@@H]4C2)C(=O)N([C@H]2CCC(=O)NC2=O)C6)CO3)ccc1[C@H]1c2ccc(O)cc2CC[C@H]1c1ccccc1] | (3S)-3-((4aS)-3-((8-(4-((1R, 2R)-6-hydroxy-2-phenyl- 1,2,3,4-tetrahydronaphthalen- 1-yl)-3-methoxyphenyl)-1-oxa- 8-azaspiro[4.5]decan-3-yl)- methyl)-8-oxo-1,2,3,4,4a,5,8, 10-octahydro-9H-pyrazino [1′,2′:4,5][1,4]oxazino[2,3-f] isoindol-9-yl)piperidine-2,6- dione |
| A57* | 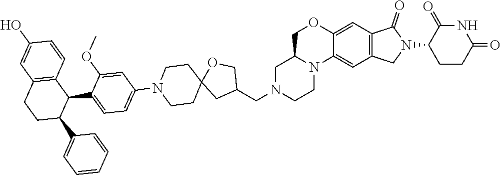 [SMILES: COc1cc(N2CCC3(CC2)CC(CN2CCN4c5cc6c(cc5OC[C@@H]4C2)C(=O)N([C@H]2CCC(=O)NC2=O)C6)CO3)ccc1[C@@H]1c2ccc(O)cc2CC[C@@H]1c1ccccc1] | (3S)-3-((4aS)-3-((8-(4-((1S,2S)- 6-hydroxy-2-phenyl-1,2,3,4- tetrahydronaphthalen-1-yl)-3- methoxyphenyl)-1-oxa-8-aza- spiro[4.5]decan-3-yl)methyl)- 8-oxo-1,2,3,4,4a,5,8,10-octa- hydro-9H-pyrazino[1′,2′:4,5] [1,4]oxazino[2,3-f]isoindol-9- yl)pipcridinc-2,6-dionc |
| A58 | 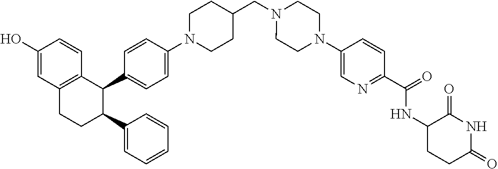 [SMILES: O=C1CCC(NC(=O)c2ccc(N3CCN(CC4CCN(c5ccc([C@@H]6c7ccc(O)cc7CC[C@@H]6c6ccccc6)cc5)CC4)CC3)cn2)C(=O)N1] | N-(2,6-dioxopiperidin-3-yl)-5- (4-((1-(4-((1R,2S)-6-hydroxy- 2-phenyl-1,2,3,4-tetrahydro- naphthalen-1-yl)phenyl)piper- idin-4-yl)methyl)piperazin-1- yl)picolinamide |
| A59* | 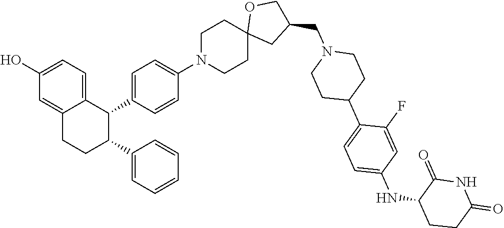 [SMILES: O=C1CC[C@H](Nc2ccc(C3CCN(C[C@@H]4COC5(CCN(c6ccc([C@H]7c8ccc(O)cc8CC[C@H]7c7ccccc7)cc6)CC5)C4)CC3)c(F)c2)C(=O)N1] | (S)-3-((3-fluoro-4-(1-(((R)-8- (4-((1S,2R)-6-hydroxy-2-phen- yl-1,2,3,4-tetrahydronaphtha- len-1-yl)phenyl)-1-oxa-8-aza- spiro[4.5]decan-3-yl)methyl)- piperidin-4-yl)phenyl)amino)- piperidine-2,6-dione |
| A60* | 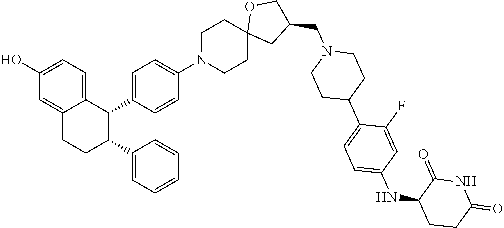 [SMILES: O=C1CC[C@@H](Nc2ccc(C3CCN(C[C@@H]4COC5(CCN(c6ccc([C@H]7c8ccc(O)cc8CC[C@H]7c7ccccc7)cc6)CC5)C4)CC3)c(F)c2)C(=O)N1] | (R)-3-((3-fluoro-4-(1-(((R)-8- (4-((1S,2R)-6-hydroxy-2- phenyl-1,2,3,4-tetrahydronaph- thalen-1-yl)phenyl)-1-oxa-8- azaspiro[4.5]decan-3-yl)meth- yl)piperidin-4-yl)phenyl)- amino)piperidine-2,6-dione |
| A61* | 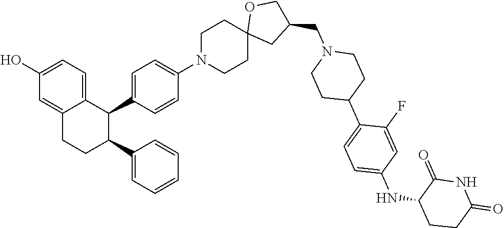 [SMILES: O=C1CC[C@H](Nc2ccc(C3CCN(C[C@@H]4COC5(CCN(c6ccc([C@@H]7c8ccc(O)cc8CC[C@@H]7c7ccccc7)cc6)CC5)C4)CC3)c(F)c2)C(=O)N1] | (S)-3-((3-fluoro-4-(1-(((R)-8- (4-((1R,2S)-6-hydroxy-2-phen- yl-1,2,3,4-tetrahydronaphtha- len-1-yl)phenyl)-1-oxa-8-aza- spiro[4.5]decan-3-yl)methyl)- piperidin-4-yl)phenyl)amino)- piperidine-2,6-dione |
| A62* | 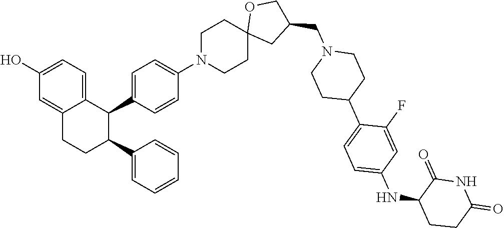 [SMILES: O=C1CC[C@@H](Nc2ccc(C3CCN(C[C@@H]4COC5(CCN(c6ccc([C@@H]7c8ccc(O)cc8CC[C@@H]7c7ccccc7)cc6)CC5)C4)CC3)c(F)c2)C(=O)N1] | (R)-3-((3-fluoro-4-(1-(((R)-8- (4-((1R,2S)-6-hydroxy-2-phen- yl-1,2,3,4-tetrahydronaphtha- len-1-yl)phenyl)-1-oxa-8-aza- spiro[4.5]decan-3-yl)methyl)- piperidin-4-yl)phenyl)amino)- piperidine-2,6-dione |
| A63* | 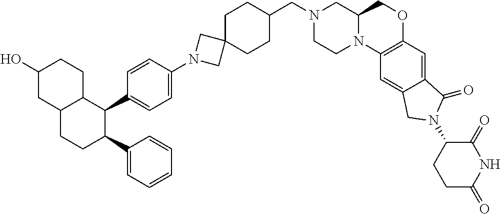 [SMILES: O=C1CC[C@H](N2Cc3cc4c(cc3C2=O)OC[C@@H]2CN(CC3CCC5(CC3)CN(c3ccc([C@@H]6C7CCC(O)CC7CC[C@@H]6c6ccccc6)cc3)C5)CCN42)C(=O)N1] | (S)-3-((S)-3-((2-(4-((1R,2S)-6- hydroxy-2-phenyl-1,2,3,4-tetra- hydronaphthalen-1-yl)phenyl)- 2-azaspiro[3.5]nonan-7-yl)- methyl)-8-oxo-3,4,4a,5-tetra- hydro-1H-pyrazino[1′,2′:4,5] [1,4]oxazino[2,3-f]isoindol- 9(2H,8H,10H)-yl)piperidine- 2,6-dione |
| A64* | 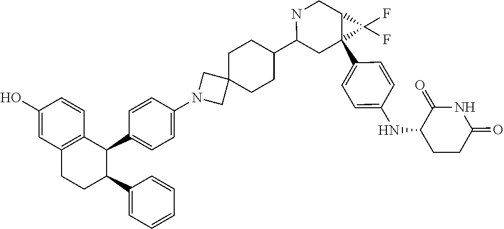 | (R)-3-((4-((1R,6R)-7,7- difluoro-3-((2-(4-((1R,2S)-6- hydroxy-2-phenyl-1,2,3,4-tetra- hydronaphthalen-1-yl)phenyl)- 2-azaspiro[3.5]nonan-7-yl)- methyl)-3-azabicyclo[4.1.0] heptan-6-yl)phenyl)amino)- piperidine-2,6-dione |
| A65* | 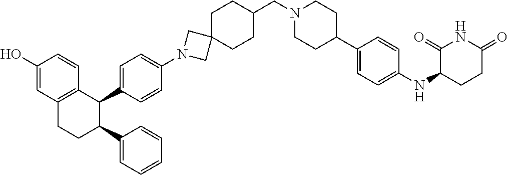 [SMILES: O=C1CC[C@@H](Nc2ccc(C3CCN(CC4CCC5(CC4)CN(c4ccc([C@@H]6c7ccc(O)cc7CC[C@@H]6c6ccccc6)cc4)C5)CC3)cc2)C(=O)N1] | (R)-3-((4-(1-((2-(4-((1R,2S)-6- hydroxy-2-phenyl-1,2,3,4-tetra- hydronaphthalen-1-yl)phenyl)- 2-azaspiro[3.5]nonan-7-yl)- methyl)piperidin-4-yl)phenyl)- amino)piperidine-2,6-dione |
| A66* | 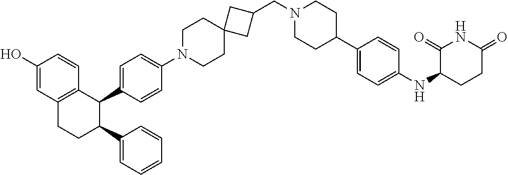 [SMILES: O=C1CC[C@@H](Nc2ccc(C3CCN(CC4CC5(CCN(c6ccc([C@@H]7c8ccc(O)cc8CC[C@@H]7c7ccccc7)cc6)CC5)C4)CC3)cc2)C(=O)N1] | (R)-3-((4-(1-((7-(4-((1R,2S)-6- hydroxy-2-phenyl-1,2,3,4-tetra- hydronaphthalen-1-yl)phenyl)- 7-azaspiro[3.5]nonan-2-yl)- methyl)piperidin-4-yl)phenyl)- amino)piperidine-2,6-dione |
| A67* | 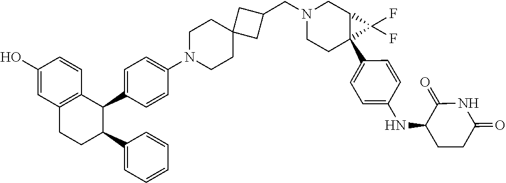 | (R)-3-((4-((1R,6R)-7,7- difluoro-3-((7-(4-((1R,2S)-6- hydroxy-2-phenyl-1,2,3,4- tetrahydronaphthalen-1-yl)- phenyl)-7-azaspiro[3.5]nonan- 2-yl)methyl)-3-azabicyclo [4.1.0]heptan-6-yl)phenyl)- amino)piperidine-2,6-dione |
| A68* | 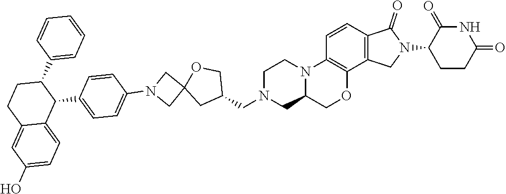 [SMILES: O=C1CC[C@H](N2Cc3c(ccc4c3OC[C@H]3CN(C[C@H]5COC6(C5)CN(c5ccc([C@@H]7c8ccc(O)cc8CC[C@@H]7c7ccccc7)cc5)C6)CCN43)C2=O)C(=O)N1] | (S)-3-((R)-7-(((S)-2-(4-((1R, 2S)-6-hydroxy-2-phenyl- 1,2,3,4-tetrahydronaphthalen- 1-yl)phenyl)-5-oxa-2-azaspiro [3.4]octan-7-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A69* | 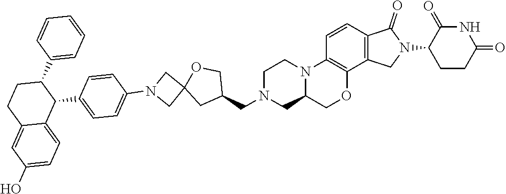 [SMILES: O=C1CC[C@H](N2Cc3c(ccc4c3OC[C@H]3CN(C[C@@H]5COC6(C5)CN(c5ccc([C@@H]7c8ccc(O)cc8CC[C@@H]7c7ccccc7)cc5)C6)CCN43)C2=O)C(=O)N1] | (S)-3-((R)-7-(((R)-2-(4-((1R, 2S)-6-hydroxy-2-phenyl- 1,2,3,4-tetrahydronaphthalen- 1-yl)phenyl)-5-oxa-2-azaspiro [3.4]octan-7-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A70 | 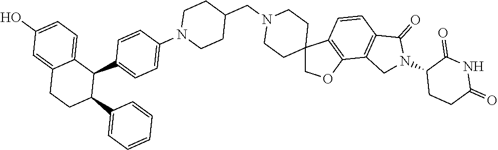 [SMILES: O=C1CC[C@H](N2Cc3c(ccc4c3OCC43CCN(CC4CCN(c5ccc([C@@H]6c7ccc(O)cc7CC[C@@H]6c6ccccc6)cc5)CC4)CC3)C2=O)C(=O)N1] | (S)-3-(1′-((1-(4-((1R,2S)-6- hydroxy-2-phenyl-1,2,3,4- tetrahydronaphthalen-1-yl)- phenyl)piperidin-4-yl)methyl)- 6-oxo-6,8-dihydro-2H,7H- spiro[furo[2,3-e]isoindole-3,4′- piperidin]-7-yl)piperidine-2,6- dione |
| A71 | 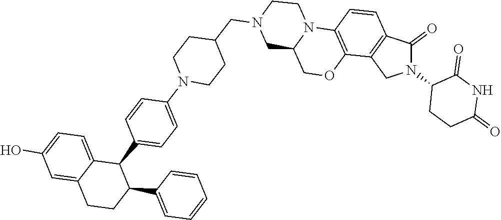 [SMILES: O=C1CC[C@H](N2Cc3c(ccc4c3OC[C@H]3CN(CC5CCN(c6ccc([C@@H]7c8ccc(O)cc8CC[C@@H]7c7ccccc7)cc6)CC5)CCN43)C2=O)C(=O)N1] | (S)-3-((R)-7-((1-(4-((1R,2S)-6- hydroxy-2-phenyl-1,2,3,4-tetra- hydronaphthalen-1-yl)phenyl)- piperidin-4-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A72 | 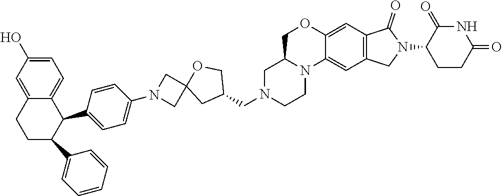 [SMILES: O=C1CC[C@H](N2Cc3cc4c(cc3C2=O)OC[C@@H]2CN(C[C@H]3COC5(C3)CN(c3ccc([C@@H]6c7ccc(O)cc7CC[C@@H]6c6ccccc6)cc3)C5)CCN42)C(=O)N1] | (S)-3-((S)-3-(((S)-2-(4-((1R, 2S)-6-hydroxy-2-phenyl- 1,2,3,4-tetrahydronaphthalen- 1-yl)phenyl)-5-oxa-2-azaspiro [3.4]octan-7-yl)methyl)-8-oxo- 1,2,3,4,4a,5,8,10-octahydro- 9H-pyrazino[1′,2′:4,5][1,4] oxazino[2,3-f]isoindol-9-yl)- piperidine-2,6-dione |
| A73 | 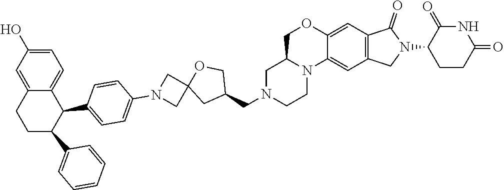 [SMILES: O=C1CC[C@H](N2Cc3cc4c(cc3C2=O)OC[C@@H]2CN(C[C@@H]3COC5(C3)CN(c3ccc([C@@H]6c7ccc(O)cc7CC[C@@H]6c6ccccc6)cc3)C5)CCN42)C(=O)N1] | (S)-3-((S)-3-(((R)-2-(4-((1R, 2S)-6-hydroxy-2-phenyl- 1,2,3,4-tetrahydronaphthalen- 1-yl)phenyl)-5-oxa-2-azaspiro [3.4]octan-7-yl)methyl)-8-oxo- 1,2,3,4,4a,5,8,10-octahydro- 9H-pyrazino[1′,2′:4,5][1,4] oxazino[2,3-f]isoindol-9-yl)- piperidine-2,6-dione |
| A74 | 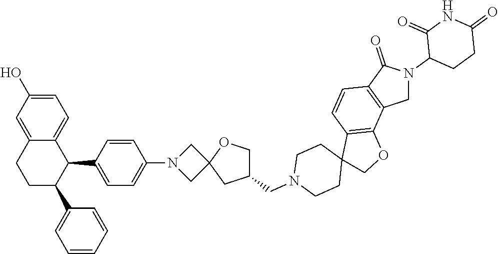 [SMILES: O=C1CCC(N2Cc3c(ccc4c3OCC43CCN(C[C@H]4COC5(C4)CN(c4ccc([C@@H]6c7ccc(O)cc7CC[C@@H]6c6ccccc6)cc4)C5)CC3)C2=O)C(=O)N1] | (S)-3-(1′-(((S)-2-(4-((1R,2S)- 6-hydroxy-2-phenyl-1,2,3,4- tetrahydronaphthalen-1-yl)- phenyl)-5-oxa-2-azaspiro[3.4] octan-7-yl)methyl)-6-oxo-6,8- dihydro-2H,7H-spiro[furo[2,3- e]isoindole-3,4′-piperidin]-7- yl)-piperidine-2,6-dione |
| A75 | 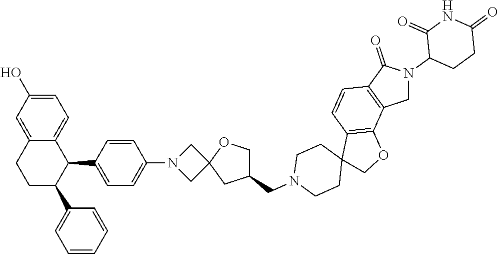 [SMILES: O=C1CCC(N2Cc3c(ccc4c3OCC43CCN(C[C@@H]4COC5(C4)CN(c4ccc([C@@H]6c7ccc(O)cc7CC[C@@H]6c6ccccc6)cc4)C5)CC3)C2=O)C(=O)N1] | (S)-3-(1′-(((R)-2-(4-((1R,2S)- 6-hydroxy-2-phenyl-1,2,3,4- tetrahydronaphthalen-1-yl)- phenyl)-5-oxa-2-azaspiro[3.4] octan-7-yl)methyl)-6-oxo-6,8- dihydro-2H,7H-spiro[furo[2,3- e]isoindole-3,4′-piperidin]-7- yl)piperidine-2,6-dione |
| A76 | 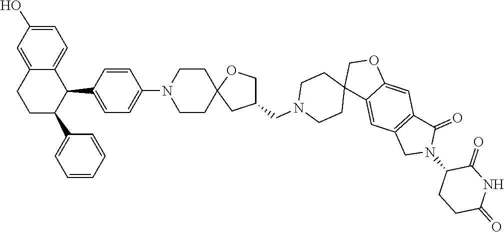 [SMILES: O=C1CC[C@H](N2Cc3cc4c(cc3C2=O)OCC42CCN(C[C@H]3COC4(CCN(c5ccc([C@@H]6c7ccc(O)cc7CC[C@@H]6c6ccccc6)cc5)CC4)C3)CC2)C(=O)N1] | (S)-3-(1′-(((S)-8-(4-((1R,2S)- 6-hydroxy-2-phenyl-1,2,3,4- tetrahydronaphthalen-1-yl)- phenyl)-1-oxa-8-azaspiro[4.5] decan-3-yl)methyl)-7-oxo-5,7- dihydro-2H,6H-spiro[furo[2,3- f]isoindole-3,4′-piperidin]-6- yl)piperidine-2,6-dione |
| A77 | 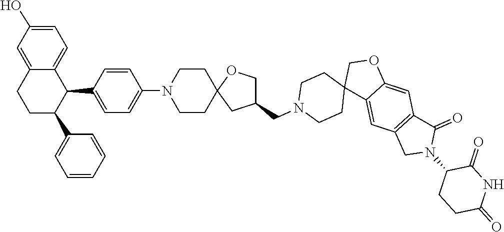 [SMILES: O=C1CC[C@H](N2Cc3cc4c(cc3C2=O)OCC42CCN(C[C@@H]3COC4(CCN(c5ccc([C@@H]6c7ccc(O)cc7CC[C@@H]6c6ccccc6)cc5)CC4)C3)CC2)C(=O)N1] | (S)-3-(1′-(((R)-8-(4-((1R,2S)- 6-hydroxy-2-phenyl-1,2,3,4- tetrahydronaphthalen-1-yl)- phenyl)-1-oxa-8-azaspiro[4.5] decan-3-yl)methyl)-7-oxo-5,7- dihydro-2H,6H-spiro[furo[2,3- f]isoindole-3,4′-piperidin]-6- yl)piperidine-2,6-dione |
| A78 | 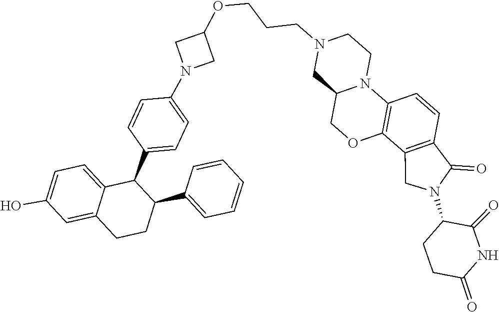 [SMILES: O=C1CC[C@H](N2Cc3c(ccc4c3OC[C@H]3CN(CCCOC5CN(c6ccc([C@@H]7c8ccc(O)cc8CC[C@@H]7c7ccccc7)cc6)C5)CCN43)C2=O)C(=O)N1] | (S)-3-((R)-7-(3-((1-(4-((1R,2S)- 6-hydroxy-2-phenyl-1,2,3,4- tetrahydronaphthalen-1-yl)- phenyl)azetidin-3-yl)oxy)prop- yl)-1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A79 | 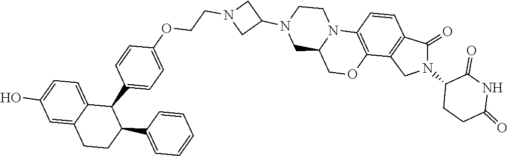 [SMILES: O=C1CC[C@H](N2Cc3c(ccc4c3OC[C@H]3CN(C5CN(CCOc6ccc([C@@H]7c8ccc(O)cc8CC[C@@H]7c7ccccc7)cc6)C5)CCN43)C2=O)C(=O)N1] | (S)-3-((R)-7-(1-(2-(4-((1R,2S)- 6-hydroxy-2-phenyl-1,2,3,4- tetrahydronaphthalen-1-yl)- phenoxy)ethyl)azetidin-3-yl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A80 | 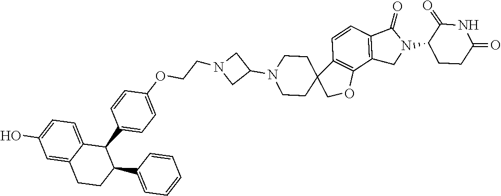 [SMILES: O=C1CC[C@H](N2Cc3c(ccc4c3OCC43CCN(C4CN(CCOc5ccc([C@@H]6c7ccc(O)cc7CC[C@@H]6c6ccccc6)cc5)C4)CC3)C2=O)C(=O)N1] | (S)-3-(1′-(1-(2-(4-((1R,2S)-6- hydroxy-2-phenyl-1,2,3,4- tetrahydronaphthalen-1-yl)- phenoxy)ethyl)azetidin-3-yl)- 6-oxo-6,8-dihydro-2H,7H- spiro[furo[2,3-c]isoindolc-3,4′- piperidin]-7-yl)piperidine-2,6- dione |
| A81 | 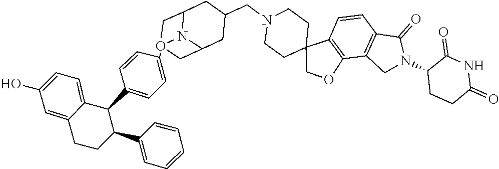 | (3S)-3-(1′-((9-(4-((1R,2S)-6- hydroxy-2-phenyl-1,2,3,4-tetra- hydronaphthalen-1-yl)phenyl)- 3-oxa-9-azabicyclo[3.3.1] nonan-7-yl)methyl)-6-oxo-6,8- dihydro-2H,7H-spiro[furo[2,3- e]isoindole-3,4′-piperidin]-7- yl)piperidine-2,6-dione |
| A82 | 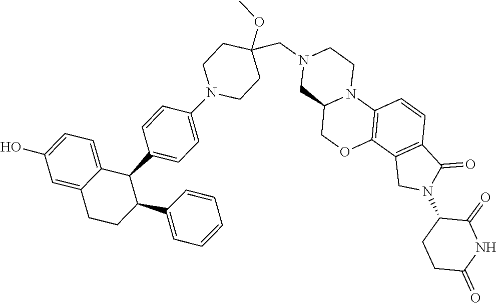 [SMILES: COC1(CN2CCN3c4ccc5c(c4OC[C@H]3C2)CN([C@H]2CCC(=O)NC2=O)C5=O)CCN(c2ccc([C@@H]3c4ccc(O)cc4CC[C@@H]3c3ccccc3)cc2)CC1] | (S)-3-((R)-7-((1-(4-((1R,2S)-6- hydroxy-2-phenyl-1,2,3,4- tetrahydronaphthalen-1-yl)- phenyl)-4-methoxypiperidin-4- yl)methyl)-1-oxo-1,3,5,5a,6,7, 8,9-octahydro-2H-pyrazino [1′,2′:4,5][1,4]oxazino[2,3-c] isoindol-2-yl)piperidine-2,6- dione |
| A83 | 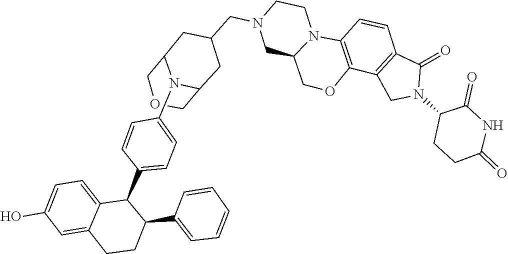 | (3S)-3-((5aR)-7-((9-(4-((1R, 2S)-6-hydroxy-2-phenyl- 1,2,3,4-tetrahydronaphthalen- 1-yl)phenyl)-3-oxa-9-azabi- cyclo[3.3.1]nonan-7-yl)meth- yl)-1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A84* | 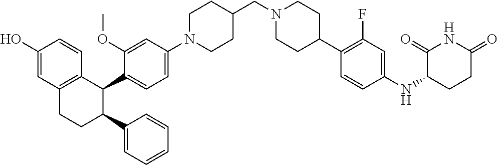 [SMILES: COc1cc(N2CCC(CN3CCC(c4ccc(N[C@H]5CCC(=O)NC5=O)cc4F)CC3)CC2)ccc1[C@@H]1c2ccc(O)cc2CC[C@@H]1c1ccccc1] | (R)-3-((3-fluoro-4-(1-((1-(4- ((1R,2R)-6-hydroxy-2-phenyl- 1,2,3,4-tetrahydronaphthalen-1- yl)-3-methoxyphenyl)piperidin- 4-yl)methyl)piperidin-4-yl)- phenyl)amino)piperidine-2,6- dione formate |
| A85* | 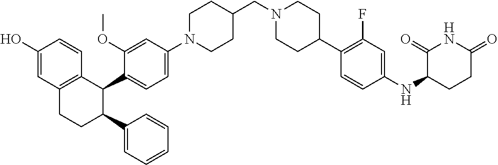 [SMILES: COc1cc(N2CCC(CN3CCC(c4ccc(N[C@@H]5CCC(=O)NC5=O)cc4F)CC3)CC2)ccc1[C@@H]1c2ccc(O)cc2CC[C@@H]1c1ccccc1] | (R)-3-((3-fluoro-4-(1-((1-(4- ((1S,2S)-6-hydroxy-2-phenyl- 1,2,3,4-tetrahydronaphthalen- 1-yl)-3-methoxyphenyl)piper- idin-4-yl)methyl)piperidin-4- yl)phenyl)amino)piperidine- 2,6-dione formate |
| A86* | 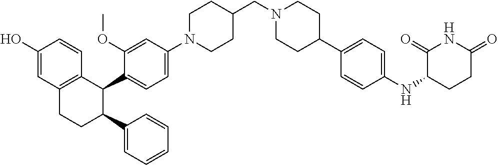 [SMILES: COc1cc(N2CCC(CN3CCC(c4ccc(N[C@H]5CCC(=O)NC5=O)cc4)CC3)CC2)ccc1[C@@H]1c2ccc(O)cc2CC[C@@H]1c1ccccc1] | (R)-3-((4-(1-((1-(4-((1R,2R)-6- hydroxy-2-phenyl-1,2,3,4-tetra- hydronaphthalen-1-yl)-3-meth- oxyphenyl)piperidin-4-yl)meth- yl)piperidin-4-l)phenyl)amino)- piperidine-2,6-dione |
| A87* | 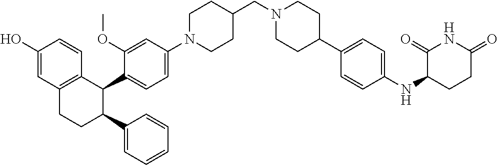 [SMILES: COc1cc(N2CCC(CN3CCC(c4ccc(N[C@@H]5CCC(=O)NC5=O)cc4)CC3)CC2)ccc1[C@@H]1c2ccc(O)cc2CC[C@@H]1c1ccccc1] | (R)-3-((4-(1-((1-(4-((1S,2S)-6- hydroxy-2-phenyl-1,2,3,4-tetra- hydronaphthalen-1-yl)-3-meth- oxyphenyl)piperidin-4-yl)meth- yl)piperidin-4-l)phenyl)amino)- piperidine-2,6-dione |
| A88* | 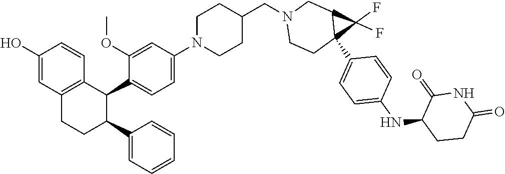 | (3R)-3-((4-((1R)-7,7-difluoro- 3-((1-(4-((1SR,2SR)-6- hydroxy-2-phenyl-1,2,3,4-tetra- hydronaphthalen-1-yl)-3-meth- oxyphenyl)piperidin-4-yl)meth- yl)-3-azabicyclo[4.1.0]heptan- 6-yl)phenyl)amino)piperidine- 2,6-dione |
| A89* | 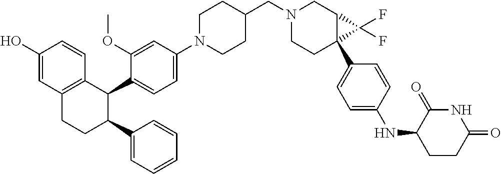 | (R)-3-((4-((1R,6R)-7,7- difluoro-3-((1-(4-((1S,2S)-6- hydroxy-2-phenyl-1,2,3,4-tetra- hydronaphthalen-1-yl)-3-meth- oxyphenyl)piperidin-4-yl)meth- yl)-3-azabicyclo[4.1.0]heptan- 6-yl)phenyl)amino)-piperidine- 2,6-dione |
| A90* | 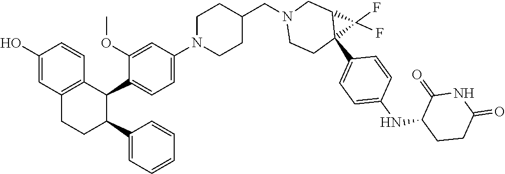 | (S)-3-((4-((1R,6R)-7,7- difluoro-3-((1-(4-((1S,2S)-6- hydroxy-2-phenyl-1,2,3,4-tetra- hydronaphthalen-1-yl)-3-meth- oxyphenyl)piperidin-4-yl)meth- yl)-3-azabicyclo[4.1.0]heptan- 6-yl)phenyl)amino)piperidine- 2,6-dione |
| A91 | 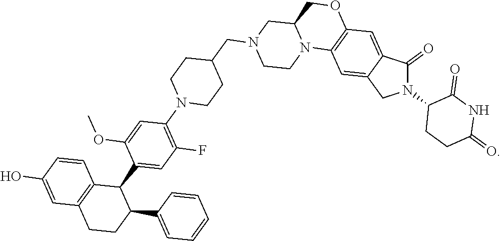 [SMILES: COc1cc(N2CCC(CN3CCN4c5cc6c(cc5OC[C@@H]4C3)C(=O)N([C@H]3CCC(=O)NC3=O)C6)CC2)c(F)cc1[C@@H]1c2ccc(O)cc2CC[C@@H]1c1ccccc1] | (S)-3-((S)-3-((1-(2-fluoro-4- ((1S,2S)-6-hydroxy-2-phenyl- 1,2,3,4-tetrahydronaphthalen-1- yl)-5-methoxyphenyl)piperidin- 4-yl)methyl)-8-oxo-1,2,3,4,4a, 5,8,10-octahydro-9H-pyrazino [1′,2′:4,5][1,4]oxazino[2,3-f] isoindol-9-yl)piperidine-2,6- dione |
| A92* | 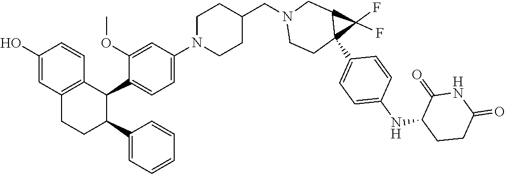 | (3S)-3-((4-((1R)-7,7-difluoro-3- ((1-(4-((1S,2S)-6-hydroxy-2- phenyl-1,2,3,4-tetrahydronaph- thalen-1-yl)-3-methoxyphenyl)- piperidin-4-yl)methyl)-3-azabi- cyclo[4.1.0]heptan-6-yl)phen- yl)amino)piperidine-2,6-dione |
| A93 | 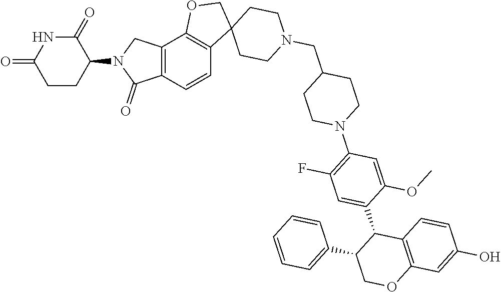 [SMILES: COc1cc(N2CCC(CN3CCC4(CC3)COc3c4ccc4c3CN([C@H]3CCC(=O)NC3=O)C4=O)CC2)c(F)cc1[C@@H]1c2ccc(O)cc2OC[C@@H]1c1ccccc1] | (S)-3-(1′-((1-(2-fluoro-4-((1S, 2S)-6-hydroxy-2-phenyl- 1,2,3,4-tetrahydronaphthalen- 1-yl)-5-methoxyphenyl)piper- idin-4-yl)methyl)-6-oxo-6,8- dihydro-2H,7H-spiro[furo[2,3- e]isoindole-3,4′-piperidin]-7- yl)piperidine-2,6-dione |
| A94 | 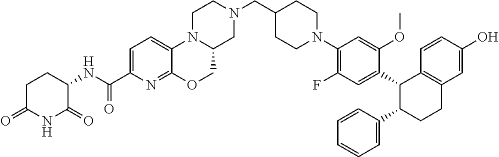 [SMILES: COc1cc(N2CCC(CN3CCN4c5ccc(C(=O)N[C@H]6CCC(=O)NC6=O)nc5OC[C@H]4C3)CC2)c(F)cc1[C@@H]1c2ccc(O)cc2CC[C@@H]1c1ccccc1] | (R)-N-((S)-2,6-dioxopiperidin- 3-yl)-3-((1-(2-fluoro-4-((1S, 2S)-6-hydroxy-2-phenyl- 1,2,3,4-tetrahydronaphthalen- 1-yl)-5-methoxyphenyl)piper- idin-4-yl)methyl)-1,2,3,4,4a,5- hexahydropyrazino[1,2-d]pyr- ido[2,3-b][1,4]oxazine-8- carboxamide |
| A95* | 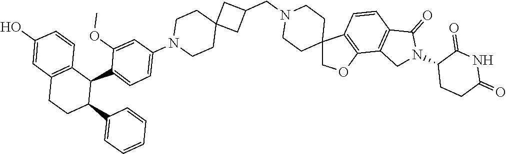 [SMILES: COc1cc(N2CCC3(CC2)CC(CN2CCC4(CC2)COc2c4ccc4c2CN([C@H]2CCC(=O)NC2=O)C4=O)C3)ccc1[C@@H]1c2ccc(O)cc2CC[C@@H]1c1ccccc1] | (S)-3-(1′-((7-(4-((1S,2S)-6- hydroxy-2-phenyl-1,2,3,4-tetra- hydronaphthalen-1-yl)-3-meth- oxyphenyl)-7-azaspiro[3.5] nonan-2-yl)methyl)-6-oxo-6,8- dihydro-2H,7H-spiro[furo[2,3- e]isoindole-3,4′-piperidin]-7- yl)piperidine-2,6-dione |
| A96* | 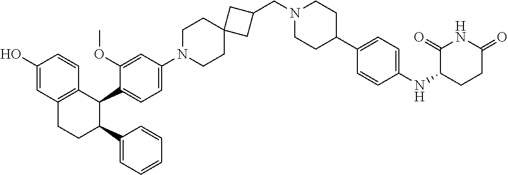 [SMILES: COc1cc(N2CCC3(CC2)CC(CN2CCC(c4ccc(N[C@H]5CCC(=O)NC5=O)cc4)CC2)C3)ccc1[C@@H]1c2ccc(O)cc2CC[C@@H]1c1ccccc1] | (S)-3-((4-(1-((7-(4-((1S,2S)-6- hydroxy-2-phenyl-1,2,3,4- tetrahydronaphthalen-1-yl)-3- methoxyphenyl)-7-azaspiro [3.5]nonan-2-yl)methyl)piper- idin-4-yl)phenyl)amino)piper- idine-2,6-dione |
| A97* | 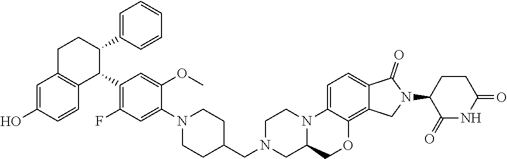 [SMILES: COc1cc([C@@H]2c3ccc(O)cc3CC[C@@H]2c2ccccc2)c(F)cc1N1CCC(CN2CCN3c4ccc5c(c4OC[C@H]3C2)CN([C@H]2CCC(=O)NC2=O)C5=O)CC1] | (S)-3-((R)-7-((1-(5-fluoro-4- ((1S,2S)-6-hydroxy-2-phenyl- 1,2,3,4-tetrahydronaphthalen-1- yl)-2-methoxyphenyl)piperidin- 4-yl)methyl)-1-oxo-1,3,5,5a,6, 7,8,9-octahydro-2H-pyrazino [1′,2′:4,5][1,4]oxazino[2,3-e] isoindol-2-yl)piperidine-2,6- dione |
| A98* | 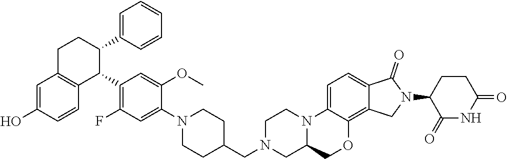 [SMILES: COc1cc([C@@H]2c3ccc(O)cc3CC[C@@H]2c2ccccc2)c(F)cc1N1CCC(CN2CCN3c4ccc5c(c4OC[C@H]3C2)CN([C@H]2CCC(=O)NC2=O)C5=O)CC1] | (S)-3-((R)-7-((1-(5-fluoro-4- ((1S,2S)-6-hydroxy-2-phenyl- 1,2,3,4-tetrahydronaphthalen-1- yl)-2-methoxyphenyl)piperidin- 4-yl)methyl)-1-oxo-1,3,5,5a,6, 7,8,9-octahydro-2H-pyrazino [1′,2′:4,5][1,4]oxazino[2,3-e] isoindol-2-yl)piperidine-2,6- dione |
| A99 | 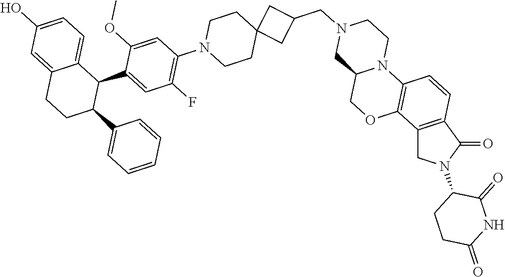 [SMILES: COc1cc(N2CCC3(CC2)CC(CN2CCN4c5ccc6c(c5OC[C@H]4C2)CN([C@H]2CCC(=O)NC2=O)C6=O)C3)c(F)cc1[C@@H]1c2ccc(O)cc2CC[C@@H]1c1ccccc1] | (S)-3-((R)-7-((7-(2-fluoro-4- ((1S,2S)-6-hydroxy-2-phenyl- 1,2,3,4-tetrahydronaphthalen-1- yl)-5-methoxyphenyl)-7-aza- spiro[3.5]nonan-2-yl)methyl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A100 | 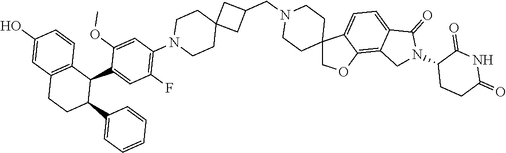 [SMILES: COc1cc(N2CCC3(CC2)CC(CN2CCC4(CC2)COc2c4ccc4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4-((1S, 2S)-6-hydroxy-2-phenyl- 1,2,3,4-tetrahydronaphthalen- 1-yl)-5-methoxyphenyl)-7-aza- spiro[3.5]nonan-2-yl)methyl)- 6-oxo-6,8-dihydro-2H,7H-spiro [furo[2,3-e]isoindole-3,4′-piper- idin]-7-yl)piperidine-2,6-dione |
| A101 | 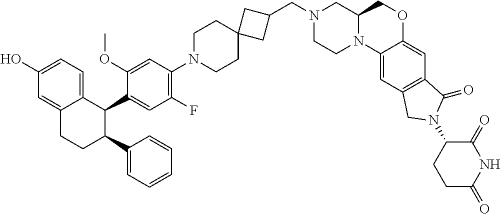 [SMILES: COc1cc(N2CCC3(CC2)CC(CN2CCN4c5cc6c(cc5OC[C@@H]4C2)C(=O)N([C@H]2CCC(=O)NC2=O)C6)C3)c(F)cc1[C@@H]1c2ccc(O)cc2CC[C@@H]1c1ccccc1] | (S)-3-((S)-3-((7-(2-fluoro-4- ((1S,2S)-6-hydroxy-2-phenyl- 1,2,3,4-tetrahydronaphthalen- 1-yl)-5-methoxyphenyl)-7-aza- spiro[3.5]nonan-2-yl)methyl)- 8-oxo-1,2,3,4,4a,5,8,10-octa- hydro-9H-pyrazino[1′,2′:4,5] [1,4]oxazino[2,3-f]isoindol-9- yl)piperidine-2,6-dione |
| A102* | 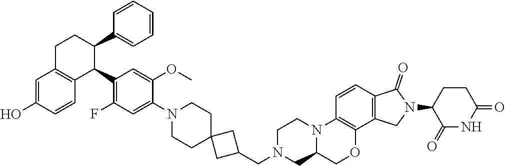 [SMILES: COc1cc([C@H]2c3ccc(O)cc3CC[C@H]2c2ccccc2)c(F)cc1N1CCC2(CC1)CC(CN1CCN3c4ccc5c(c4OC[C@H]3C1)CN([C@H]1CCC(=O)NC1=O)C5=O)C2] | (S)-3-((R)-7-((7-(5-fluoro-4- ((1R,2R)-6-hydroxy-2-phenyl- 1,2,3,4-tetrahydronaphthalen- 1-yl)-2-methoxyphenyl)-7-aza- spiro[3.5]nonan-2-yl)methyl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A103* | 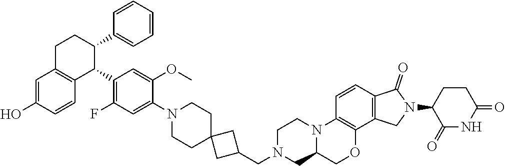 [SMILES: COc1cc([C@@H]2c3ccc(O)cc3CC[C@@H]2c2ccccc2)c(F)cc1N1CCC2(CC1)CC(CN1CCN3c4ccc5c(c4OC[C@H]3C1)CN([C@H]1CCC(=O)NC1=O)C5=O)C2] | (S)-3-((R)-7-((7-(5-fluoro-4- ((1S,2S)-6-hydroxy-2-phenyl- 1,2,3,4-tetrahydronaphthalen- 1-yl)-2-methoxyphenyl)-7-aza- spiro[3.5]nonan-2-yl)methyl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A104 | 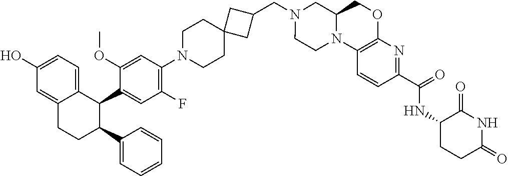 [SMILES: COc1cc(N2CCC3(CC2)CC(CN2CCN4c5ccc(C(=O)N[C@H]6CCC(=O)NC6=O)nc5OC[C@@H]4C2)C3)c(F)cc1[C@@H]1c2ccc(O)cc2CC[C@@H]1c1ccccc1] | (S)-N-((S)-2,6-dioxopiperidin- 3-yl)-3-((7-(2-fluoro-4-((1S, 2S)-6-hydroxy-2-phenyl-1,2, 3,4-tetrahydronaphthalen-1-yl)- 5-methoxyphenyl)-7-azaspiro [3.5]nonan-2-yl)methyl)-1,2,3, 4,4a,5-hexahydropyrazino[1,2- d]pyrido[2,3-b][1,4]oxazine-8- carboxamide |
| A105* | 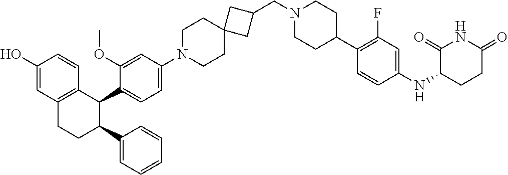 [SMILES: COc1cc(N2CCC3(CC2)CC(CN2CCC(c4ccc(N[C@H]5CCC(=O)NC5=O)cc4F)CC2)C3)ccc1[C@@H]1c2ccc(O)cc2CC[C@@H]1c1ccccc1] | (S)-3-((3-fluoro-4-(1-((7-(4- ((1S,2S)-6-hydroxy-2-phenyl- 1,2,3,4-tetrahydronaphthalen- 1-yl)-3-methoxyphenyl)-7-aza- spiro[3.5]nonan-2-yl)methyl)- piperidin-4-yl)phenyl)amino)- piperidine-2,6-dione |
| A106* | 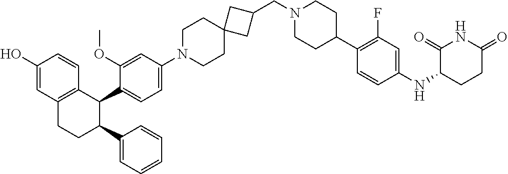 [SMILES: COc1cc(N2CCC3(CC2)CC(CN2CCC(c4ccc(N[C@H]5CCC(=O)NC5=O)cc4F)CC2)C3)ccc1[C@@H]1c2ccc(O)cc2CC[C@@H]1c1ccccc1] | (R)-3-((3-fluoro-4-(1-((7-(4- ((1S,2S)-6-hydroxy-2-phenyl- 1,2,3,4-tetrahydronaphthalen- 1-yl)-3-methoxyphenyl)-7-aza- spiro[3.5]nonan-2-yl)methyl)- piperidin-4-yl)phenyl)amino)- piperidine-2,6-dione |
| A107* | 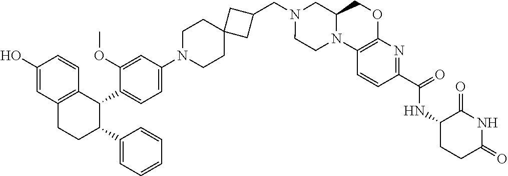 [SMILES: COc1cc(N2CCC3(CC2)CC(CN2CCN4c5ccc(C(=O)N[C@H]6CCC(=O)NC6=O)nc5OC[C@@H]4C2)C3)ccc1[C@H]1c2ccc(O)cc2CC[C@H]1c1ccccc1] | (S)-N-((S)-2,6-dioxopiperidin- 3-yl)-3-((7-(4-((1R,2R)-6- hydroxy-2-phenyl-1,2,3,4-tetra- hydronaphthalen-1-yl)-3-meth- oxyphenyl)-7-azaspiro[3.5] nonan-2-yl)methyl)-1,2,3,4, 4a,5-hcxahydropyrazino[1,2-d] pyrido[2,3-b][1,4]oxazine-8- carboxamide |
| A108* | 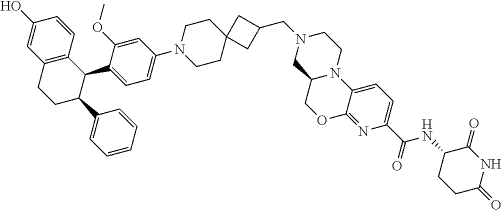 [SMILES: COc1cc(N2CCC3(CC2)CC(CN2CCN4c5ccc(C(=O)N[C@H]6CCC(=O)NC6=O)nc5OC[C@H]4C2)C3)ccc1[C@@H]1c2ccc(O)cc2CC[C@@H]1c1ccccc1] | (S)-N-((S)-2,6-dioxopiperidin- 3-yl)-3-((7-(4-((1S,2S)-6- hydroxy-2-phenyl-1,2,3,4-tetra- hydronaphthalen-1-yl)-3-meth- oxyphenyl)-7-azaspiro[3.5] nonan-2-yl)methyl)-1,2,3,4,4a, 5-hexahydropyrazino[1,2-d] pyrido[2,3-b][1,4]oxazine-8- carboxamide |
| A109* | 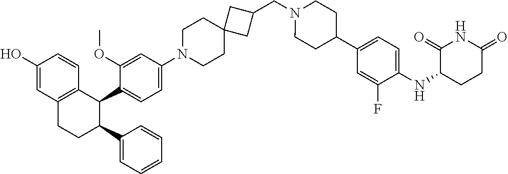 [SMILES: COc1cc(N2CCC3(CC2)CC(CN2CCC(c4ccc(N[C@H]5CCC(=O)NC5=O)c(F)c4)CC2)C3)ccc1[C@@H]1c2ccc(O)cc2CC[C@@H]1c1ccccc1] | 2-((S)-2,6-dioxopiperidin-3- yl)-5-(1-((7-(5-((1S,2S)-6- droxy-2-phenyl-1,2,3,4-tetra- hydronaphthalen-1-yl)pyridin- 2-yl)-7-azaspiro[3.5]nonan-2- yl)methyl)piperidin-4-yl)-3,5- dihydro-1H-pyrrolo[3,4-c]pyr- idine-1,4(2H)-dione |
| A110* | 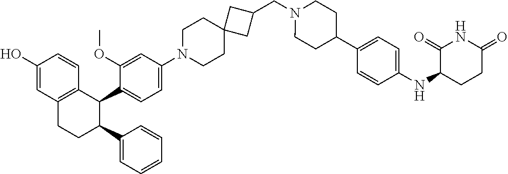 [SMILES: COc1cc(N2CCC3(CC2)CC(CN2CCC(c4ccc(N[C@@H]5CCC(=O)NC5=O)cc4)CC2)C3)ccc1[C@@H]1c2ccc(O)cc2CC[C@@H]1c1ccccc1] | (R)-3-((4-(1-((7-(4-((1S,2S)-6- hydroxy-2-phenyl-1,2,3,4- tetrahydronaphthalen-1-yl)-3- methoxyphenyl)-7-azaspiro [3.5]nonan-2-yl)methyl)piper- idin-4-yl)phenyl)amino)piper- idine-2,6-dione |
| A111 | 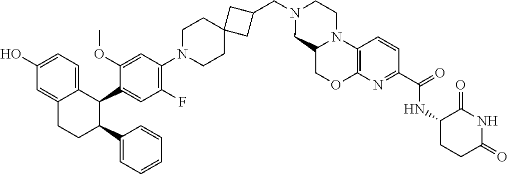 | (R)-N-((S)-2,6-dioxopiperidin- 3-yl)-3-((7-(2-fluoro-4-((1S, 2S)-6-hydroxy-2-phenyl- 1,2,3,4-tetrahydronaphthalen- 1-yl)-5-methoxyphenyl)-7- azaspiro[3.5]nonan-2-yl)meth- yl)-1,2,3,4,4a,5-hexahydro- pyrazino[1,2-d]pyrido[2,3-b] [1,4]oxazine-8-carboxamide |
| A112* | 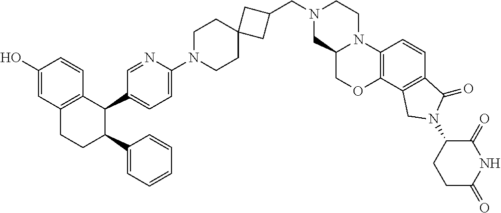 [SMILES: O=C1CC[C@H](N2Cc3c(ccc4c3OC[C@H]3CN(CC5CC6(CCN(c7ccc([C@@H]8c9ccc(O)cc9CC[C@@H]8c8ccccc8)cn7)CC6)C5)CCN43)C2=O)C(=O)N1] | (S)-3-((R)-7-((7-(5-((1S,2S)-6- hydroxy-2-phenyl-1,2,3,4-tetra- hydronaphthalen-1-yl)pyridin- 2-yl)-7-azaspiro[3.5]nonan-2- yl)methyl)-1-oxo-1,3,5,5a,6,7, 8,9-octahydro-2H-pyrazino [1′,2′:4,5][1,4]oxazino[2,3-e] isoindol-2-yl)piperidine-2,6- dione |
| A113* | 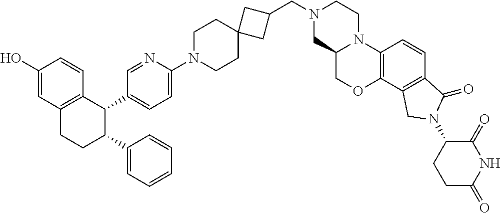 [SMILES: O=C1CC[C@H](N2Cc3c(ccc4c3OC[C@H]3CN(CC5CC6(CCN(c7ccc([C@H]8c9ccc(O)cc9CC[C@H]8c8ccccc8)cn7)CC6)C5)CCN43)C2=O)C(=O)N1] | (S)-3-((R)-7-((7-(5-((1R,2R)-6- hydroxy-2-phenyl-1,2,3,4-tetra- hydronaphthalen-1-yl)pyridin- 2-yl)-7-azaspiro[3.5]nonan-2- yl)methyl)-1-oxo-1,3,5,5a,6,7, 8,9-octahydro-2H-pyrazino [1′,2′:4,5][1,4]oxazino[2,3-c] isoindol-2-yl)piperidine-2,6- dione |
| A114* | 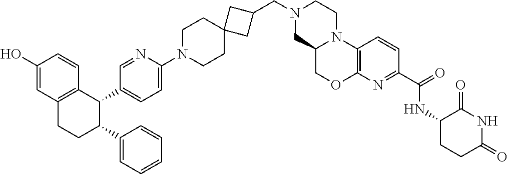 [SMILES: O=C1CC[C@H](NC(=O)c2ccc3c(n2)OC[C@H]2CN(CC4CC5(CCN(c6ccc([C@H]7c8ccc(O)cc8CC[C@H]7c7ccccc7)cn6)CC5)C4)CCN32)C(=O)N1] | (R)-N-((S)-2,6-dioxopiperidin- 3-yl)-3-((7-(5-((1R,2R)-6- hydroxy-2-phenyl-1,2,3,4-tetra- hydronaphthalen-1-yl)pyridin- 2-yl)-7-azaspiro[3.5]nonan-2- yl)mcthyl)-1,2,3,4,4a,5-hexa- hydropyrazino[1,2-d]pyrido [2,3-b][1,4]oxazine-8-carbox- amide |
| A115* | 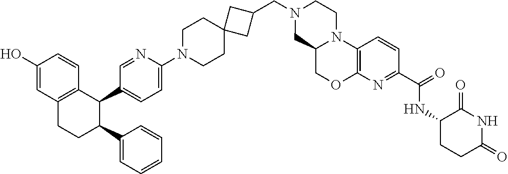 [SMILES: O=C1CC[C@H](NC(=O)c2ccc3c(n2)OC[C@H]2CN(CC4CC5(CCN(c6ccc([C@@H]7c8ccc(O)cc8CC[C@@H]7c7ccccc7)cn6)CC5)C4)CCN32)C(=O)N1] | (R)-N-((S)-2,6-dioxopiperidin- 3-yl)-3-((7-(5-((1S,2S)-6- hydroxy-2-phenyl-1,2,3,4-tetra- hydronaphthalen-1-yl)pyridin- 2-yl)-7-azaspiro[3.5]nonan-2- yl)methyl)-1,2,3,4,4a,5-hexa- hydropyrazino[1,2-d]pyrido [2,3-b][1,4]oxazine-8-carbox- amide |
| A116* | 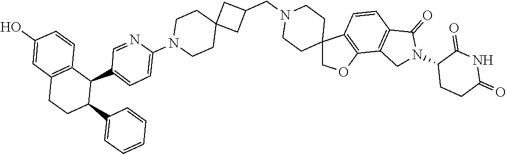 [SMILES: O=C1CC[C@H](N2Cc3c(ccc4c3OCC43CCN(CC4CC5(CCN(c6ccc([C@@H]7c8ccc(O)cc8CC[C@@H]7c7ccccc7)cn6)CC5)C4)CC3)C2=O)C(=O)N1] | (S)-3-(1′-((7-(5-((1S,2S)-6- hydroxy-2-phenyl-1,2,3,4-tetra- hydronaphthalen-1-yl)pyridin- 2-yl)-7-azaspiro[3.5]nonan-2- yl)methyl)-6-oxo-6,8-dihydro- 2H,7H-spiro[furo[2,3-e]iso- indole-3,4′-piperidin]-7-yl)- piperidine-2,6-dione |
| A117* | 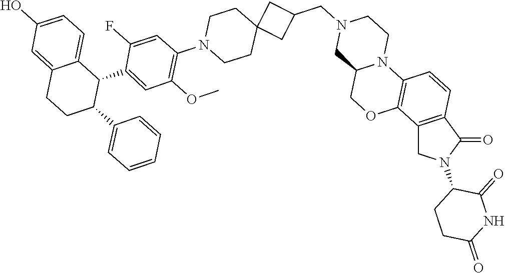 [SMILES: COc1cc([C@H]2c3ccc(O)cc3CC[C@H]2c2ccccc2)c(F)cc1N1CCC2(CC1)CC(CN1CCN3c4ccc5c(c4OC[C@H]3C1)CN([C@H]1CCC(=O)NC1=O)C5=O)C2] | (S)-3-((R)-7-((7-(5-fluoro-4- ((1R,2R)-6-hydroxy-2-phenyl- 1,2,3,4-tetrahydronaphthalen-1- yl)-2-methoxyphenyl)-7-aza- spiro[3.5]nonan-2-yl)methyl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A118 | 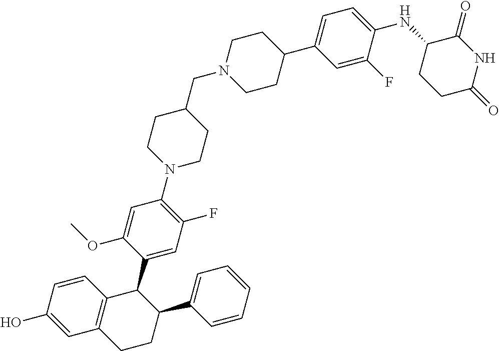 [SMILES: COc1cc(N2CCC(CN3CCC(c4ccc(N[C@H]5CCC(=O)NC5=O)c(F)c4)CC3)CC2)c(F)cc1[C@@H]1c2ccc(O)cc2CC[C@@H]1c1ccccc1] | (S)-3-((2-fluoro-4-(1-((1-(2- fluoro-4-((1S,2S)-6-hydroxy-2- phenyl-1,2,3,4-tetrahydronaph- thalen-1-yl)-5-methoxyphenyl)- piperidin-4-yl)methyl)piper- idin-4-yl)phenyl)amino)piper- idine-2,6-dione |
| A119 | 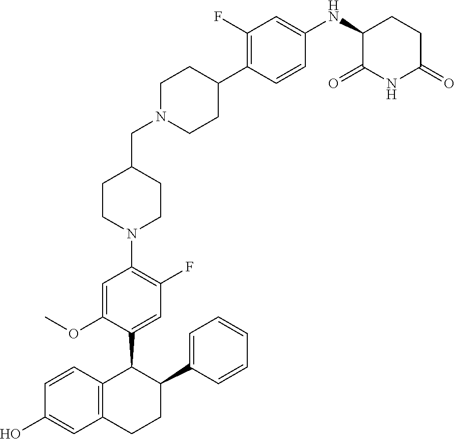 [SMILES: COc1cc(N2CCC(CN3CCC(c4ccc(N[C@H]5CCC(=O)NC5=O)cc4F)CC3)CC2)c(F)cc1[C@@H]1c2ccc(O)cc2CC[C@@H]1c1ccccc1] | (S)-3-((3-fluoro-4-(1-((1-(2- fluoro-4-((1S,2S)-6-hydroxy-2- phenyl-1,2,3,4-tetrahydronaph- thalen-1-yl)-5-methoxyphenyl)- piperidin-4-yl)methyl)piper- idin-4-yl)phenyl)amino)piper- idine-2,6-dione |
| A120* | 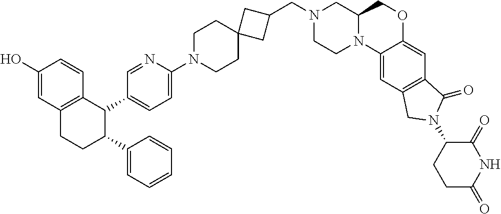 | (S)-3-((S)-3-((7-(5-((1R,2R)-6- hydroxy-2-phenyl-1,2,3,4-tetra- hydronaphthalen-1-yl)pyridin- 2-yl)-7-azaspiro[3.5]nonan-2- yl)methyl)-8-oxo-3,4,4a,5-tetra- hydro-1H-pyrazino[1′,2′:4,5] [1,4]oxazino[2,3-f]isoindol- 9(2H,8H,10H)-yl)piperidine- 2,6-dione |
| A121* | 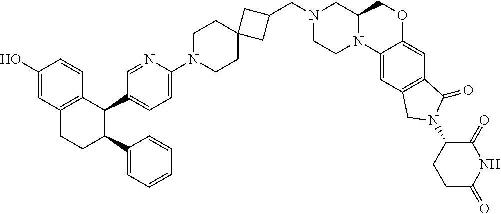 [SMILES: O=C1CC[C@H](N2Cc3cc4c(cc3C2=O)OC[C@@H]2CN(CC3CC5(CCN(c6ccc([C@@H]7c8ccc(O)cc8CC[C@@H]7c7ccccc7)cn6)CC5)C3)CCN42)C(=O)N1] | (S)-3-((S)-3-((7-(5-((1S,2S)-6- hydroxy-2-phenyl-1,2,3,4-tetra- hydronaphthalen-1-yl)pyridin- 2-yl)-7-azaspiro[3.5]nonan-2- yl)methyl)-8-oxo-1,2,3,4,4a,5, 8,10-octahydro-9H-pyrazino [1′,2′:4,5][1,4]oxazino[2,3-f] isoindol-9-yl)piperidine-2,6- dione |
| A122 | 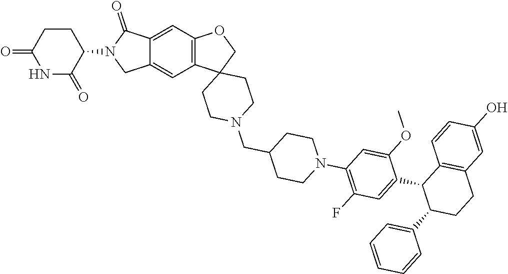 [SMILES: COc1cc(N2CCC(CN3CCC4(CC3)COc3cc5c(cc34)CN([C@H]3CCC(=O)NC3=O)C5=O)CC2)c(F)cc1[C@@H]1c2ccc(O)cc2CC[C@@H]1c1ccccc1] | (S)-3-(1′-((1-(2-fluoro-4-((1S, 2S)-6-hydroxy-2-phenyl- 1,2,3,4-tetrahydronaphthalen- 1-yl)-5-methoxyphenyl)piper- idin-4-yl)methyl)-7-oxo-5,7- dihydro-2H,6H-spiro[furo[2,3- f]isoindole-3,4′-piperidin]-6- yl)piperidine-2,6-dione |
| A123* | 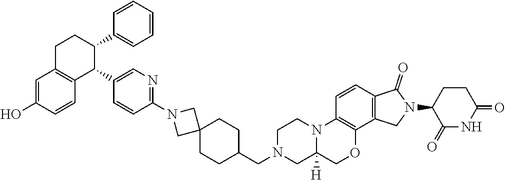 [SMILES: O=C1CC[C@H](N2Cc3c(ccc4c3OC[C@H]3CN(CC5CCC6(CC5)CN(c5ccc([C@@H]7c8ccc(O)cc8CC[C@@H]7c7ccccc7)cn5)C6)CCN43)C2=O)C(=O)N1] | (S)-3-((R)-7-((2-(5-((1S,2S)-6- hydroxy-2-phenyl-1,2,3,4-tetra- hydronaphthalcn-1-yl)pyridin- 2-yl)-2-azaspiro[3.5]nonan-7- yl)methyl)-1-oxo-1,3,5,5a,6,7, 8,9-octahydro-2H-pyrazino [1′,2′:4,5][1,4]oxazino[2,3-e] isoindol-2-yl)piperidine-2,6- dione |
| A124* | 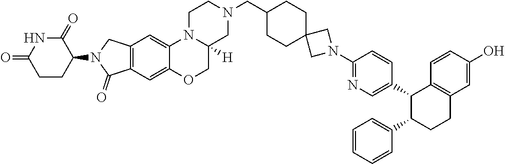 [SMILES: O=C1CC[C@H](N2Cc3cc4c(cc3C2=O)OC[C@@H]2CN(CC3CCC5(CC3)CN(c3ccc([C@@H]6c7ccc(O)cc7CC[C@@H]6c6ccccc6)cn3)C5)CCN42)C(=O)N1] | (S)-3-((S)-3-((2-(5-((1S,2S)-6- hydroxy-2-phenyl-1,2,3,4-tetra- hydronaphthalen-1-yl)pyridin- 2-yl)-2-azaspiro[3.5]nonan-7- yl)methyl)-8-oxo-1,2,3,4,4a,5, 8,10-octahydro-9H-pyrazino [1′,2′:4,5][1,4]oxazino[2,3-f] isoindol-9-yl)piperidine-2,6- dione |
| A125* | 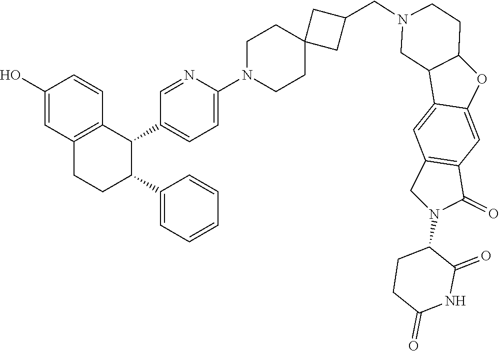 [SMILES: O=C1CC[C@H](N2Cc3cc4c(cc3C2=O)OC2CCN(CC3CC5(CCN(c6ccc([C@H]7c8ccc(O)cc8CC[C@H]7c7ccccc7)cn6)CC5)C3)CC42)C(=O)N1] | (S)-3-(1′-((7-(5-((1R,2R)-6- hydroxy-2-phenyl-1,2,3,4-tetra- hydronaphthalen-1-yl)pyridin- 2-yl)-7-azaspiro[3.5]nonan-2- yl)methyl)-7-oxo-5,7-dihydro- 2H,6H-spiro[furo[2,3-f]iso- indole-3,4′-piperidin]-6-yl)- piperidine-2,6-dionc |
| A126* | 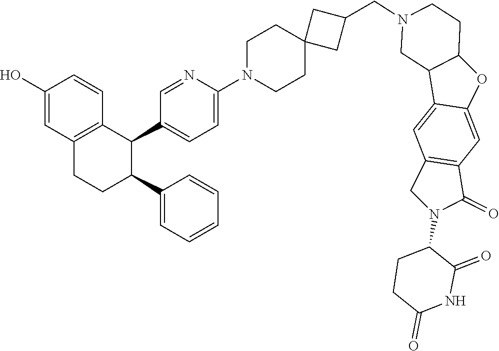 [SMILES: O=C1CC[C@H](N2Cc3cc4c(cc3C2=O)OC2CCN(CC3CC5(CCN(c6ccc([C@@H]7c8ccc(O)cc8CC[C@@H]7c7ccccc7)cn6)CC5)C3)CC42)C(=O)N1] | (S)-3-(1′-((7-(5-((1S,2S)-6- hydroxy-2-phenyl-1,2,3,4-tetra- hydronaphthalen-1-yl)pyridin- 2-yl)-7-azaspiro[3.5]nonan-2- yl)methyl)-7-oxo-5,7-dihydro- 2H,6H-spiro[furo[2,3-f]iso- indole-3,4′-piperidin]-6-yl)- piperidine-2,6-dione |
| A127* | 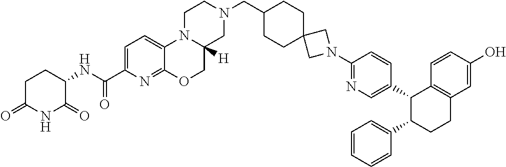 [SMILES: O=C1CC[C@H](NC(=O)c2ccc3c(n2)OC[C@H]2CN(CC4CCC5(CC4)CN(c4ccc([C@@H]6c7ccc(O)cc7CC[C@@H]6c6ccccc6)cn4)C5)CCN32)C(=O)N1] | (R)-N-((S)-2,6-dioxopiperidin- 3-yl)-3-((2-(5-((1S,2S)-6- hydroxy-2-phenyl-1,2,3,4-tetra- hydronaphthalen-1-yl)pyridin- 2-yl)-2-azaspiro[3.5]nonan-7- yl)methyl)-1,2,3,4,4a,5-hexa- hydropyrazino[1,2-d]pyrido [2,3-b][1,4]oxazine-8-carbox- amide |
| A128* | 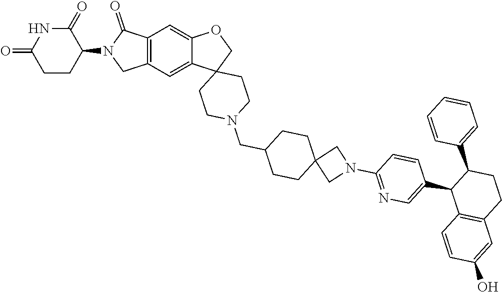 | (S)-3-(1′-((2-(5-((1S,2S)-6- hydroxy-2-phenyl-1,2,3,4-tetra- hydronaphthalen-1-yl)pyridin- 2-yl)-2-azaspiro[3.5]nonan-7- yl)methyl)-7-oxo-5,7-dihydro- 2H,6H-spiro[furo[2,3-f]iso- indole-3,4′-piperidin]-6-yl)- piperidine-2,6-dione |
| A129* | 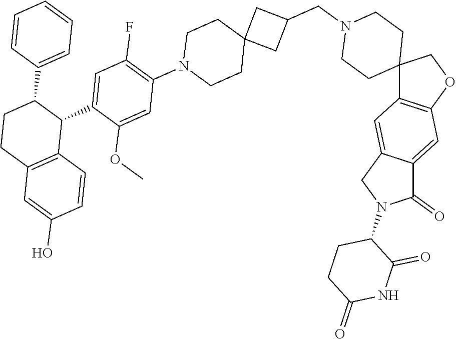 [SMILES: COc1cc(N2CCC3(CC2)CC(CN2CCC4(CC2)COc2cc5c(cc24)CN([C@H]2CCC(=O)NC2=O)C5=O)C3)c(F)cc1[C@@H]1c2ccc(O)cc2CC[C@@H]1c1ccccc1] | (S)-3-(1′-((7-(2-fluoro-4-((1S, 2S)-6-hydroxy-2-phenyl- 1,2,3,4-tetrahydronaphthalen- 1-yl)-5-methoxyphenyl)-7-aza- spiro[3.5]nonan-2-yl)methyl)- 7-oxo-5,7-dihydro-2H,6H- spiro[furo[2,3-f]isoindole-3,4′- piperidin]-6-yl)piperidine-2,6- dione |
| A130* | 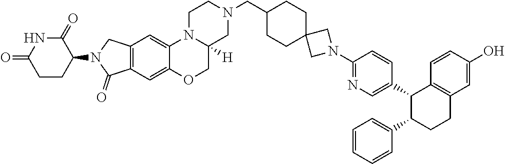 [SMILES: O=C1CC[C@H](N2Cc3cc4c(cc3C2=O)OC[C@@H]2CN(CC3CCC5(CC3)CN(c3ccc([C@@H]6c7ccc(O)cc7CC[C@@H]6c6ccccc6)cn3)C5)CCN42)C(=O)N1] | (S)-3-((S)-3-((2-(5-((1S,2S)-6- hydroxy-2-phenyl-1,2,3,4-tetra- hydronaphthalcn-1-yl)pyridin- 2-yl)-2-azaspiro[3.5]nonan-7- yl)methyl)-8-oxo-1,2,3,4,4a,5, 8,10-octahydro-9H-pyrazino [1′,2′:4,5][1,4]oxazino[2,3-f] isoindol-9-yl)piperidine-2,6- dione |
| A131* | 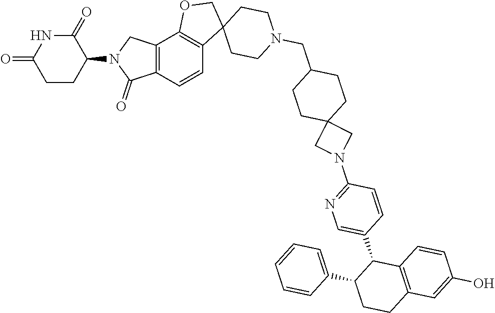 [SMILES: O=C1CC[C@H](N2Cc3c(ccc4c3OCC43CCN(CC4CCC5(CC4)CN(c4ccc([C@@H]6c7ccc(O)cc7CC[C@@H]6c6ccccc6)cn4)C5)CC3)C2=O)C(=O)N1] | (S)-3-(1′-((2-(5-((1S,2S)-6- hydroxy-2-phenyl-1,2,3,4-tetra- hydronaphthalen-1-yl)pyridin- 2-yl)-2-azaspiro[3.5]nonan-7- yl)methyl)-6-oxo-6,8-dihydro- 2H,7H-spiro[furo[2,3-e]iso- indole-3,4′-piperidin]-7-yl)- piperidine-2,6-dione |
| A132* | 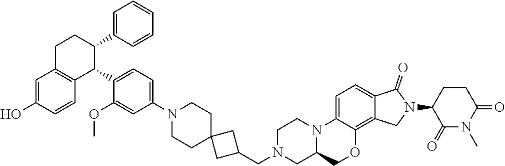 [SMILES: COc1cc(N2CCC3(CC2)CC(CN2CCN4c5ccc6c(c5OC[C@H]4C2)CN([C@H]2CCC(=O)N(C)C2=O)C6=O)C3)ccc1[C@@H]1c2ccc(O)cc2CC[C@@H]1c1ccccc1] | (S)-3-((R)-7-((7-(4-((1S,2S)-6- hydroxy-2-phenyl-1,2,3,4-tetra- hydronaphthalen-1-yl)-3-meth- oxyphenyl)-7-azaspiro[3.5] nonan-2-yl)methyl)-1-oxo- 1,3,5,5a,6,7,8,9-octahydro-2H- pyrazino[1′,2′:4,5][1,4]oxazino [2,3-e]isoindol-2-yl)-1-methyl- piperidine-2,6-dione |
| A133* | 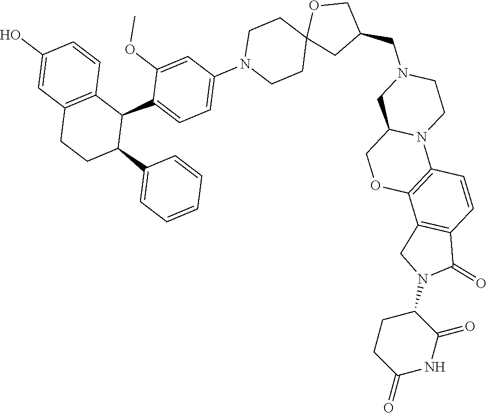 [SMILES: COc1cc(N2CCC3(CC2)C[C@H](CN2CCN4c5ccc6c(c5OC[C@H]4C2)CN([C@H]2CCC(=O)NC2=O)C6=O)CO3)ccc1[C@@H]1c2ccc(O)cc2CC[C@@H]1c1ccccc1] | (S)-3-((R)-7-(((R)-8-(4-((1S, 2S)-6-hydroxy-2-phenyl- 1,2,3,4-tetrahydronaphthalen- 1-yl)-3-methoxyphenyl)-1-oxa- 8-azaspiro[4.5]decan-3-yl)- methyl)-1-oxo-1,3,5,5a,6,7,8,9- octahydro-2H-pyrazino[1′,2′: 4,5][1,4]oxazino[2,3-e]isoindol- 2-yl)piperidine-2,6-dione |
| A134* | 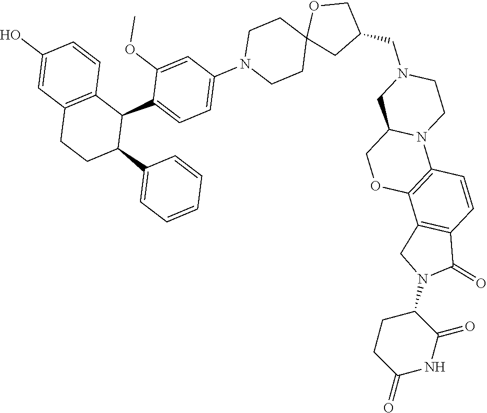 [SMILES: COc1cc(N2CCC3(CC2)C[C@@H](CN2CCN4c5ccc6c(c5OC[C@H]4C2)CN([C@H]2CCC(=O)NC2=O)C6=O)CO3)ccc1[C@@H]1c2ccc(O)cc2CC[C@@H]1c1ccccc1] | (S)-3-((R)-7-(((S)-8-(4-((1S, 2S)-6-hydroxy-2-phenyl- 1,2,3,4-tetrahydronaphthalen- 1-yl)-3-methoxyphenyl)-1-oxa- 8-azaspiro[4.5]decan-3-yl)- methyl)-1-oxo-1,3,5,5a,6,7,8,9- octahydro-2H-pyrazino[1′,2′: 4,5][1,4]oxazino[2,3-e]isoindol- 2-yl)piperidine-2,6-dione |
| A135* | 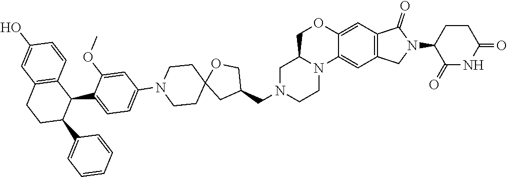 [SMILES: COc1cc(N2CCC3(CC2)C[C@H](CN2CCN4c5cc6c(cc5OC[C@@H]4C2)C(=O)N([C@H]2CCC(=O)NC2=O)C6)CO3)ccc1[C@@H]1c2ccc(O)cc2CC[C@@H]1c1ccccc1] | (S)-3-((S)-3-(((R)-8-(4-((1S, 2S)-6-hydroxy-2-phenyl- 1,2,3,4-tetrahydronaphthalen- 1-yl)-3-methoxyphenyl)-1-oxa- 8-azaspiro[4.5]decan-3-yl)- methyl)-8-oxo-1,2,3,4,4a,5,8, 10-octahydro-9H-pyrazino [1′,2′:4,5][1,4]oxazino[2,3-f] isoindol-9-yl)piperidine-2,6- dione |
| A136* | 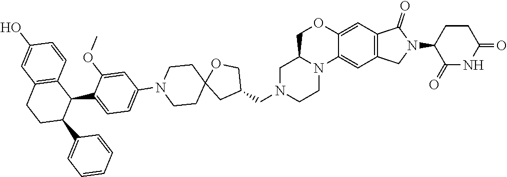 [SMILES: COc1cc(N2CCC3(CC2)C[C@@H](CN2CCN4c5cc6c(cc5OC[C@@H]4C2)C(=O)N([C@H]2CCC(=O)NC2=O)C6)CO3)ccc1[C@@H]1c2ccc(O)cc2CC[C@@H]1c1ccccc1] | (S)-3-((S)-3-(((S)-8-(4-((1S, 2S)-6-hydroxy-2-phenyl- 1,2,3,4-tetrahydronaphthalen- 1-yl)-3-methoxyphenyl)-1-oxa- 8-azaspiro[4.5]decan-3-yl)- methyl)-8-oxo-1,2,3,4,4a,5,8, 10-octahydro-9H-pyrazino [1′,2′:4,5][1,4]oxazino[2,3-f] isoindol-9-yl)piperidine-2,6- dione |
| A137* | 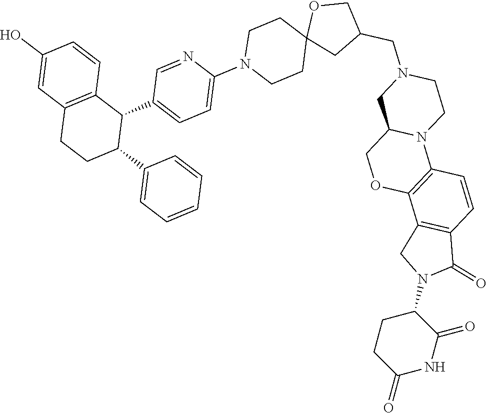 [SMILES: O=C1CC[C@H](N2Cc3c(ccc4c3OC[C@H]3CN(CC5COC6(CCN(c7ccc([C@H]8c9ccc(O)cc9CC[C@H]8c8ccccc8)cn7)CC6)C5)CCN43)C2=O)C(=O)N1] | (3S)-3-((5aR)-7-((8-(5-((1R, 2R)-6-hydroxy-2-phenyl- 1,2,3,4-tetrahydronaphthalen- 1-yl)pyridin-2-yl)-1-oxa-8-aza- spiro[4.5]decan-3-yl)methyl)-1- oxo-1,3,5,5a,6,7,8,9-octahydro- 2H-pyrazino[1′,2′:4,5][1,4] oxazino[2,3-e]isoindol-2-yl)- piperidine-2,6-dione |
| A138* | 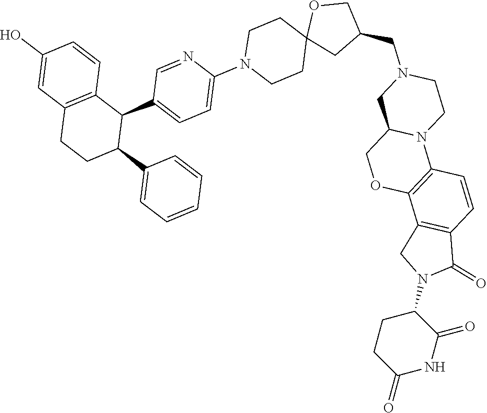 [SMILES: O=C1CC[C@H](N2Cc3c(ccc4c3OC[C@H]3CN(C[C@@H]5COC6(CCN(c7ccc([C@@H]8c9ccc(O)cc9CC[C@@H]8c8ccccc8)cn7)CC6)C5)CCN43)C2=O)C(=O)N1] | (3S)-3-((5aR)-7-((8-(5-((1S, 2S)-6-hydroxy-2-phenyl- 1,2,3,4-tetrahydronaphthalen- 1-yl)pyridin-2-yl)-1-oxa-8-aza- spiro[4.5]decan-3-yl)methyl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A139* | 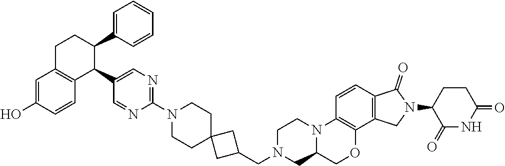 [SMILES: O=C1CC[C@H](N2Cc3c(ccc4c3OC[C@H]3CN(CC5CC6(CCN(c7ncc([C@H]8c9ccc(O)cc9CC[C@H]8c8ccccc8)cn7)CC6)C5)CCN43)C2=O)C(=O)N1] | (S)-3-((R)-7-((7-(5-((1R,2R)-6- hydroxy-2-phenyl-1,2,3,4-tetra- hydronaphthalen-1-yl)pyrimi- din-2-yl)-7-azaspiro[3.5]nonan- 2-yl)methyl)-1-oxo-1,3,5,5a,6, 7,8,9-octahydro-2H-pyrazino [1′,2′:4,5][1,4]oxazino[2,3-e] isoindol-2-yl)piperidine-2,6- dione |
| A140* | 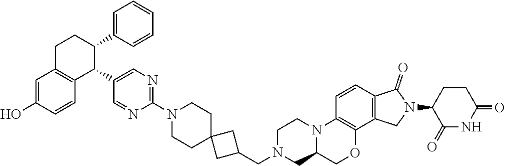 [SMILES: O=C1CC[C@H](N2Cc3c(ccc4c3OC[C@H]3CN(CC5CC6(CCN(c7ncc([C@@H]8c9ccc(O)cc9CC[C@@H]8c8ccccc8)cn7)CC6)C5)CCN43)C2=O)C(=O)N1] | (S)-3-((R)-7-((7-(5-((1S,2S)-6- hydroxy-2-phenyl-1,2,3,4-tetra- hydronaphthalen-1-yl)pyrimi- din-2-yl)-7-azaspiro[3.5]nonan- 2-yl)methyl)-1-oxo-1,3,5,5a,6, 7,8,9-octahydro-2H-pyrazino [1′,2′:4,5][1,4]oxazino[2,3-e] isoindol-2-yl)piperidine-2,6- dione |
| A141* | 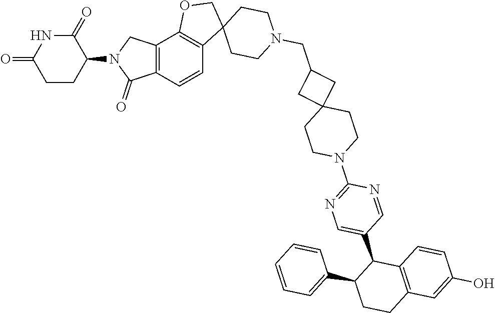 [SMILES: O=C1CC[C@H](N2Cc3c(ccc4c3OCC43CCN(CC4CC5(CCN(c6ncc([C@H]7c8ccc(O)cc8CC[C@H]7c7ccccc7)cn6)CC5)C4)CC3)C2=O)C(=O)N1] | (S)-3-(1′-((7-(5-((1R,2R)-6- hydroxy-2-phenyl-1,2,3,4- tetrahydronaphthalen-1-yl)- pyrimidin-2-yl)-7-azaspiro[3.5] nonan-2-yl)methyl)-6-oxo-6,8- dihydro-2H,7H-spiro[furo[2,3- c]isoindolc-3,4′-piperidin]-7- yl)piperidine-2,6-dione |
| A142* | 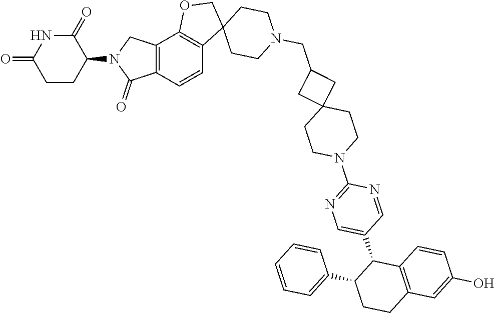 [SMILES: O=C1CC[C@H](N2Cc3c(ccc4c3OCC43CCN(CC4CC5(CCN(c6ncc([C@@H]7c8ccc(O)cc8CC[C@@H]7c7ccccc7)cn6)CC5)C4)CC3)C2=O)C(=O)N1] | (S)-3-(1′-((7-(5-((1S,2S)-6- hydroxy-2-phenyl-1,2,3,4- tetrahydronaphthalen-1-yl)pyr- imidin-2-yl)-7-azaspiro[3.5] nonan-2-yl)methyl)-6-oxo-6,8- dihydro-2H,7H-spiro[furo[2,3- e]isoindole-3,4′-piperidin]-7- yl)piperidine-2,6-dione |
| A143* | 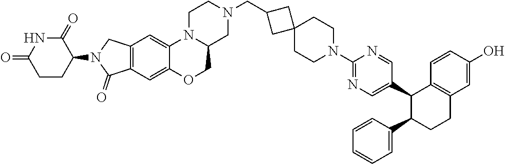 [SMILES: O=C1CC[C@H](N2Cc3cc4c(cc3C2=O)OC[C@@H]2CN(CC3CC5(CCN(c6ncc([C@H]7c8ccc(O)cc8CC[C@H]7c7ccccc7)cn6)CC5)C3)CCN42)C(=O)N1] | (S)-3-((S)-3-((7-(5-((1R,2R)-6- hydroxy-2-phenyl-1,2,3,4-tetra- hydronaphthalen-1-yl)pyrimi- din-2-yl)-7-azaspiro[3.5]nonan- 2-yl)methyl)-8-oxo-1,2,3,4,4a, 5,8,10-octahydro-9H-pyrazino [1′,2′:4,5][1,4]oxazino[2,3-f] isoindol-9-yl)piperidine-2,6- dione |
| A144* | 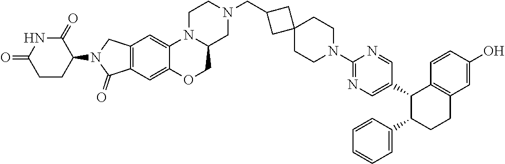 [SMILES: O=C1CC[C@H](N2Cc3cc4c(cc3C2=O)OC[C@@H]2CN(CC3CC5(CCN(c6ncc([C@@H]7c8ccc(O)cc8CC[C@@H]7c7ccccc7)cn6)CC5)C3)CCN42)C(=O)N1] | (S)-3-((S)-3-((7-(5-((1S,2S)-6- hydroxy-2-phenyl-1,2,3,4-tetra- hydronaphthalen-1-yl)pyrimi- din-2-yl)-7-azaspiro[3.5]nonan- 2-yl)mcthyl)-8-oxo-1,2,3,4,4a, 5,8,10-octahydro-9H-pyrazino [1′,2′:4,5][1,4]oxazino[2,3-f] isoindol-9-yl)piperidine-2,6- dione |
| A145* | 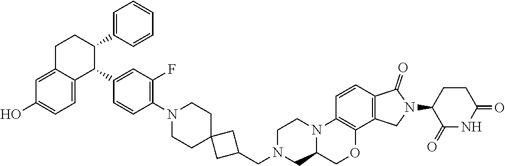 [SMILES: O=C1CC[C@H](N2Cc3c(ccc4c3OC[C@H]3CN(CC5CC6(CCN(c7ccc([C@@H]8c9ccc(O)cc9CC[C@@H]8c8ccccc8)cc7F)CC6)C5)CCN43)C2=O)C(=O)N1] | (S)-3-((R)-7-((7-(2-fluoro-4- ((1R,2S)-6-hydroxy-2-phenyl- 1,2,3,4-tetrahydronaphthalen- 1-yl)phenyl)-7-azaspiro[3.5] nonan-2-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A146* | 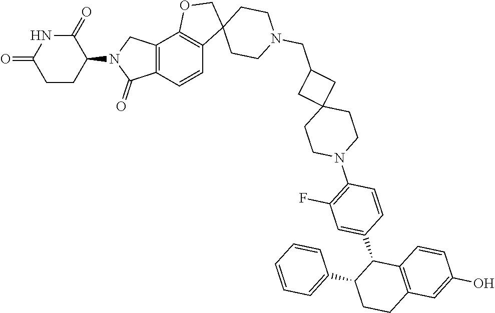 [SMILES: O=C1CC[C@H](N2Cc3c(ccc4c3OCC43CCN(CC4CC5(CCN(c6ccc([C@@H]7c8ccc(O)cc8CC[C@@H]7c7ccccc7)cc6F)CC5)C4)CC3)C2=O)C(=O)N1] | (S)-3-(1′-((7-(2-fluoro-4-((1R, 2S)-6-hydroxy-2-phenyl- 1,2,3,4-tetrahydronaphthalen- 1-yl)phenyl)-7-azaspiro[3.5] nonan-2-yl)methyl)-6-oxo-6,8- dihydro-2H,7H-spiro[furo[2,3- e]isoindole-3,4′-piperidin]-7- yl)piperidine-2,6-dione |
| A147* | 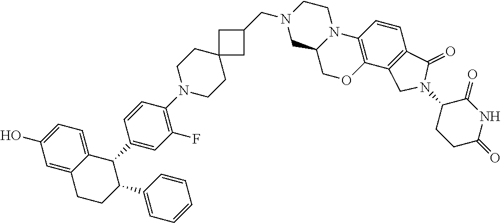 [SMILES: O=C1CC[C@H](N2Cc3c(ccc4c3OC[C@H]3CN(CC5CC6(CCN(c7ccc([C@H]8c9ccc(O)cc9CC[C@H]8c8ccccc8)cc7F)CC6)C5)CCN43)C2=O)C(=O)N1] | (S)-3-((R)-7-((7-(2-fluoro-4- ((1S,2R)-6-hydroxy-2-phenyl- 1,2,3,4-tetrahydronaphthalen- 1-yl)phenyl)-7-azaspiro[3.5] nonan-2-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A148* | 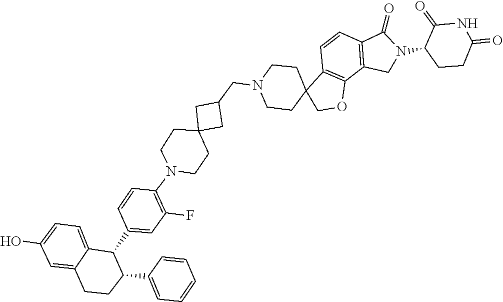 [SMILES: O=C1CC[C@H](N2Cc3c(ccc4c3OCC43CCN(CC4CC5(CCN(c6ccc([C@H]7c8ccc(O)cc8CC[C@H]7c7ccccc7)cc6F)CC5)C4)CC3)C2=O)C(=O)N1] | (S)-3-(1′-((7-(2-fluoro-4-((1S, 2R)-6-hydroxy-2-phenyl- 1,2,3,4-tetrahydronaphthalen- 1-yl)phenyl)-7-azaspiro[3.5] nonan-2-yl)methyl)-6-oxo-6,8- dihydro-2H,7H-spiro[furo[2,3- e]isoindole-3,4′-piperidin]-7- yl)piperidine-2,6-dione |
| A149* | 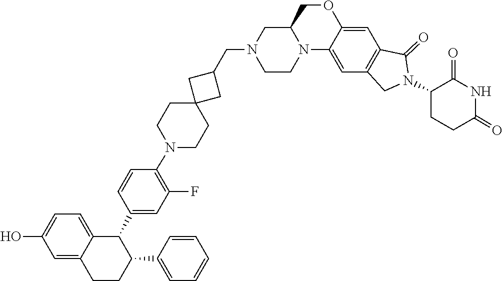 [SMILES: O=C1CC[C@H](N2Cc3cc4c(cc3C2=O)OC[C@@H]2CN(CC3CC5(CCN(c6ccc([C@H]7c8ccc(O)cc8CC[C@H]7c7ccccc7)cc6F)CC5)C3)CCN42)C(=O)N1] | (S)-3-((S)-3-((7-(2-fluoro-4- ((1S,2R)-6-hydroxy-2-phenyl- 1,2,3,4-tetrahydronaphthalen- 1-yl)phenyl)-7-azaspiro[3.5] nonan-2-yl)methyl)-8-oxo- 1,2,3,4,4a,5,8,10-octahydro- 9H-pyrazino[1′,2′:4,5][1,4] oxazino[2,3-f]isoindol-9-yl)- piperidine-2,6-dione |
| A150* | 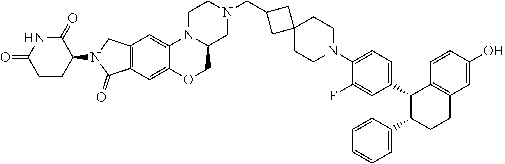 [SMILES: O=C1CC[C@H](N2Cc3cc4c(cc3C2=O)OC[C@@H]2CN(CC3CC5(CCN(c6ccc([C@@H]7c8ccc(O)cc8CC[C@@H]7c7ccccc7)cc6F)CC5)C3)CCN42)C(=O)N1] | (S)-3-((S)-3-((7-(2-fluoro-4- ((1R,2S)-6-hydroxy-2-phenyl- 1,2,3,4-tetrahydronaphthalen- 1-yl)phenyl)-7-azaspiro[3.5] nonan-2-yl)methyl)-8-oxo- 1,2,3,4,4a,5,8,10-octahydro- 9H-pyrazino[1′,2′:4,5][1,4] oxazino[2,3-f]isoindol-9-yl)- piperidine-2,6-dione |
| A151* | 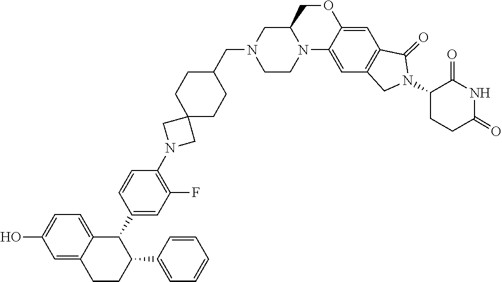 [SMILES: O=C1CC[C@H](N2Cc3cc4c(cc3C2=O)OC[C@@H]2CN(CC3CCC5(CC3)CN(c3ccc([C@H]6c7ccc(O)cc7CC[C@H]6c6ccccc6)cc3F)C5)CCN42)C(=O)N1] | (S)-3-((S)-3-((2-(2-fluoro-4- ((1S,2R)-6-hydroxy-2-phenyl- 1,2,3,4-tetrahydronaphthalen- 1-yl)phenyl)-2-azaspiro[3.5] nonan-7-yl)methyl)-8-oxo- 1,2,3,4,4a,5,8,10-octahydro- 9H-pyrazino[1′,2′:4,5][1,4] oxazino[2,3-f]isoindol-9-yl)- piperidine-2,6-dione |
| A152* | 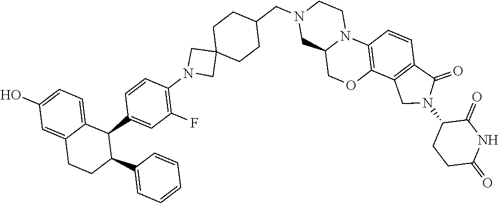 [SMILES: O=C1CC[C@H](N2Cc3c(ccc4c3OC[C@H]3CN(CC5CCC6(CC5)CN(c5ccc([C@@H]7c8ccc(O)cc8CC[C@@H]7c7ccccc7)cc5F)C6)CCN43)C2=O)C(=O)N1] | (S)-3-((R)-7-((2-(2-fluoro-4- ((1R,2S)-6-hydroxy-2-phenyl- 1,2,3,4-tetrahydronaphthalen- 1-yl)phenyl)-2-azaspiro[3.5] nonan-7-yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A153* | 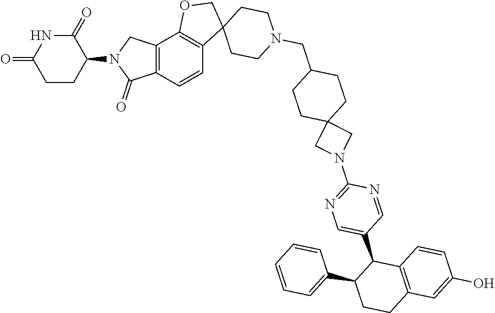 [SMILES: O=C1CC[C@H](N2Cc3c(ccc4c3OCC43CCN(CC4CCC5(CC4)CN(c4ncc([C@H]6c7ccc(O)cc7CC[C@H]6c6ccccc6)cn4)C5)CC3)C2=O)C(=O)N1] | (S)-3-(1′-((2-(5-((1R,2R)-6- hydroxy-2-phenyl-1,2,3,4- tetrahydronaphthalen-1-yl)pyr- imidin-2-yl)-2-azaspiro[3.5] nonan-7-yl)methyl)-6-oxo-6,8- dihydro-2H,7H-spiro[furo[2,3- e]isoindole-3,4′-piperidin]-7- yl)piperidine-2,6-dione |
| A154* | 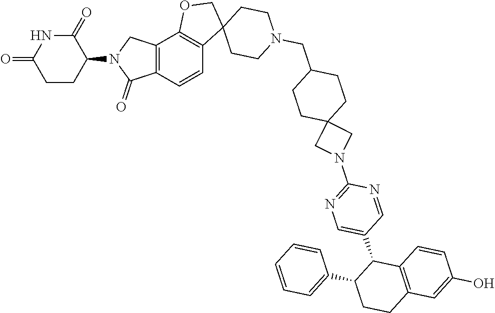 [SMILES: O=C1CC[C@H](N2Cc3c(ccc4c3OCC43CCN(CC4CCC5(CC4)CN(c4ncc([C@@H]6c7ccc(O)cc7CC[C@@H]6c6ccccc6)cn4)C5)CC3)C2=O)C(=O)N1] | (S)-3-(1′-((2-(5-((1S,2S)-6- hydroxy-2-phenyl-1,2,3,4- tetrahydronaphthalen-1-yl)pyr- imidin-2-yl)-2-azaspiro[3.5] nonan-7-yl)methyl)-6-oxo-6,8- dihydro-2H,7H-spiro[furo[2,3- e]isoindole-3,4′-piperidin]-7- yl)piperidine-2,6-dione |
| A155* | 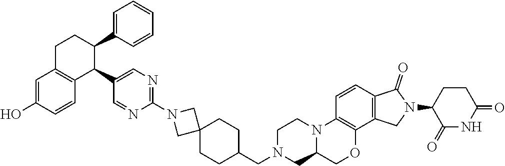 [SMILES: O=C1CC[C@H](N2Cc3c(ccc4c3OC[C@H]3CN(CC5CCC6(CC5)CN(c5ncc([C@H]7c8ccc(O)cc8CC[C@H]7c7ccccc7)cn5)C6)CCN43)C2=O)C(=O)N1] | (S)-3-((R)-7-((2-(5-((1R,2R)-6- hydroxy-2-phenyl-1,2,3,4-tetra- hydronaphthalen-1-yl)pyrimi- din-2-yl)-2-azaspiro[3.5]nonan- 7-yl)methyl)-1-oxo-1,3,5,5a,6, 7,8,9-octahydro-2H-pyrazino [1′,2′:4,5][1,4]oxazino[2,3-e] isoindol-2-yl)piperidine-2,6- dione |
| A156* | 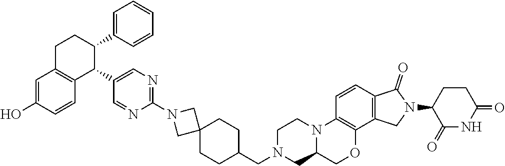 [SMILES: O=C1CC[C@H](N2Cc3c(ccc4c3OC[C@H]3CN(CC5CCC6(CC5)CN(c5ncc([C@@H]7c8ccc(O)cc8CC[C@@H]7c7ccccc7)cn5)C6)CCN43)C2=O)C(=O)N1] | (S)-3-((R)-7-((2-(5-((1S,2S)-6- hydroxy-2-phenyl-1,2,3,4-tetra- hydronaphthalen-1-yl)pyrimi- din-2-yl)-2-azaspiro[3.5]nonan- 7-yl)methyl)-1-oxo-1,3,5,5a,6, 7,8,9-octahydro-2H-pyrazino [1′,2′:4,5][1,4]oxazino[2,3-e] isoindol-2-yl)piperidine-2,6- dione |
| A157* | 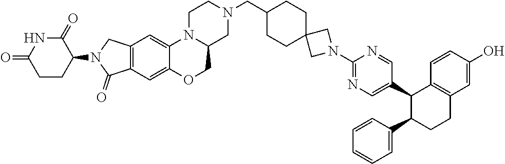 [SMILES: O=C1CC[C@H](N2Cc3cc4c(cc3C2=O)OC[C@@H]2CN(CC3CCC5(CC3)CN(c3ncc([C@H]6c7ccc(O)cc7CC[C@H]6c6ccccc6)cn3)C5)CCN42)C(=O)N1] | (S)-3-((S)-3-((2-(5-((1R,2R)-6- hydroxy-2-phenyl-1,2,3,4-tetra- hydronaphthalen-1-yl)pyrimi- din-2-yl)-2-azaspiro[3.5]nonan- 7-yl)methyl)-8-oxo-1,2,3,4,4a, 5,8,10-octahydro-9H-pyrazino [1′,2′:4,5][1,4]oxazino[2,3-f] isoindol-9-yl)piperidine-2,6- dione |
| A158* | 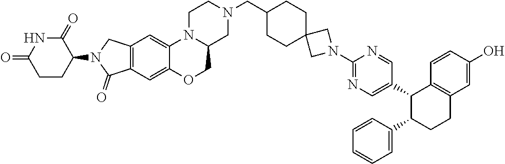 [SMILES: O=C1CC[C@H](N2Cc3cc4c(cc3C2=O)OC[C@@H]2CN(CC3CCC5(CC3)CN(c3ncc([C@@H]6c7ccc(O)cc7CC[C@@H]6c6ccccc6)cn3)C5)CCN42)C(=O)N1] | (S)-3-((S)-3-((2-(5-((1S,2S)-6- hydroxy-2-phenyl-1,2,3,4-tetra- hydronaphthalen-1-yl)pyrimi- din-2-yl)-2-azaspiro[3.5]nonan- 7-yl)methyl)-8-oxo-1,2,3,4,4a, 5,8,10-octahydro-9H-pyrazino [1′,2′:4,5][1,4]oxazino[2,3-f] isoindol-9-yl)piperidine-2,6- dione |
| A159* | 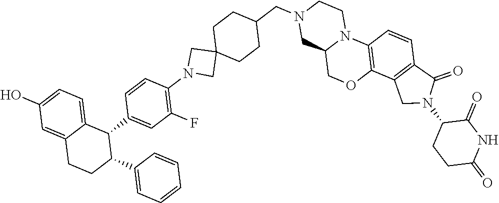 [SMILES: O=C1CC[C@H](N2Cc3c(ccc4c3OC[C@H]3CN(CC5CCC6(CC5)CN(c5ccc([C@H]7c8ccc(O)cc8CC[C@H]7c7ccccc7)cc5F)C6)CCN43)C2=O)C(=O)N1] | (S)-3-((R)-7-((2-(2-fluoro-4- ((1S,2R)-6-hydroxy-2-phenyl- 1,2,3,4-tetrahydronaphthalen- 1-yl)phenyl)-2-azaspiro[3.5] nonan-7-yl)methyl)-1-oxo-5,5a, 6,7,8,9-hcxahydro-1H-pyrazino [1′,2′:4,5][1,4]oxazino[2,3-e] isoindol-2(3H)-yl)piperidine- 2,6-dione |
| A160* | 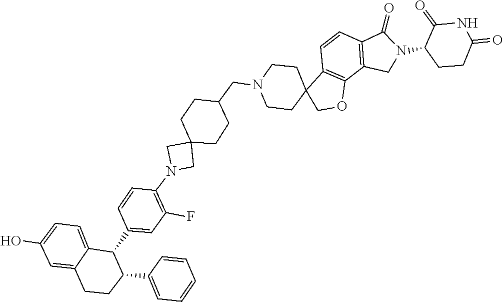 [SMILES: O=C1CC[C@H](N2Cc3c(ccc4c3OCC43CCN(CC4CCC5(CC4)CN(c4ccc([C@H]6c7ccc(O)cc7CC[C@H]6c6ccccc6)cc4F)C5)CC3)C2=O)C(=O)N1] | (S)-3-(1′-((2-(2-fluoro-4-((1S, 2R)-6-hydroxy-2-phenyl- 1,2,3,4-tetrahydronaphthalen- 1-yl)phenyl)-2-azaspiro[3.5] nonan-7-yl)methyl)-6-oxo-6,8- dihydro-2H,7H-spiro[furo[2,3- c]isoindolc-3,4′-pipcridin]-7- yl)pipcridinc-2,6-dione |
| A161* | 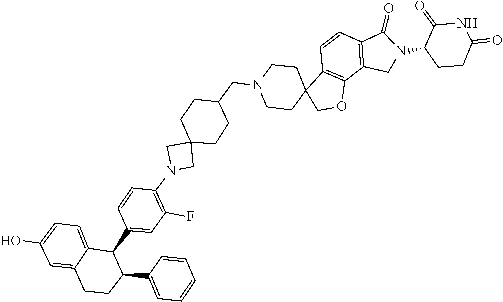 [SMILES: O=C1CC[C@H](N2Cc3c(ccc4c3OCC43CCN(CC4CCC5(CC4)CN(c4ccc([C@@H]6c7ccc(O)cc7CC[C@@H]6c6ccccc6)cc4F)C5)CC3)C2=O)C(=O)N1] | (S)-3-(1′-((2-(2-fluoro-4-((1R, 2S)-6-hydroxy-2-phenyl- 1,2,3,4-tetrahydronaphthalen- 1-yl)phenyl)-2-azaspiro[3.5] nonan-7-yl)methyl)-6-oxo-6,8- dihydro-2H,7H-spiro[furo[2,3- e]isoindole-3,4′-piperidin]-7- yl)piperidine-2,6-dione |
| A162* | 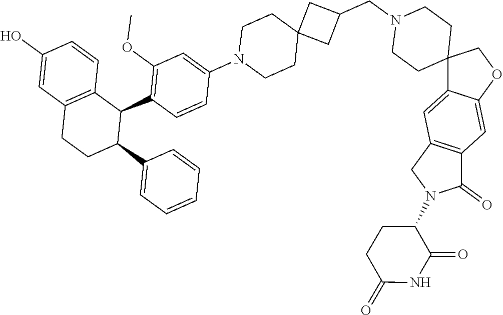 [SMILES: COc1cc(N2CCC3(CC2)CC(CN2CCC4(CC2)COc2cc5c(cc24)CN([C@H]2CCC(=O)NC2=O)C5=O)C3)ccc1[C@@H]1c2ccc(O)cc2CC[C@@H]1c1ccccc1] | (S)-3-(1′-((7-(4-((1S,2S)-6- hydroxy-2-phenyl-1,2,3,4- tetrahydronaphthalen-1-yl)-3- methoxyphenyl)-7-azaspiro [3.5]nonan-2-yl)methyl)-7-oxo- 5,7-dihydro-2H,6H-spiro[furo [2,3-f]isoindole-3,4′-piperidin]- 6-yl)piperidine-2,6-dione |
| A163* | 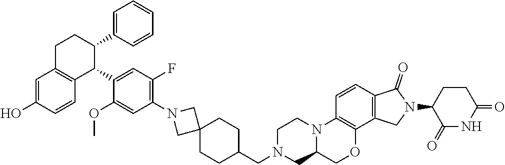 [SMILES: COc1cc(N2CC3(CCC(CN4CCN5c6ccc7c(c6OC[C@H]5C4)CN([C@H]4CCC(=O)NC4=O)C7=O)CC3)C2)c(F)cc1[C@@H]1c2ccc(O)cc2CC[C@@H]1c1ccccc1] | (S)-3-((R)-7-((2-(2-fluoro-4- ((1S,2S)-6-hydroxy-2-phenyl- 1,2,3,4-tetrahydronaphthalen- 1-yl)-5-methoxyphenyl)-2-aza- spiro[3.5]nonan-7-yl)methyl)- 1-oxo-1,3,5,5a,6,7,8,9-octa- hydro-2H-pyrazino[1′,2′:4,5] [1,4]oxazino[2,3-e]isoindol- 2-yl)piperidine-2,6-dione |
| A164* | 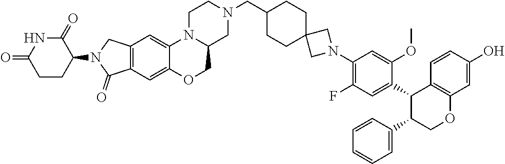 [SMILES: COc1cc(N2CC3(CCC(CN4CCN5c6cc7c(cc6OC[C@@H]5C4)C(=O)N([C@H]4CCC(=O)NC4=O)C7)CC3)C2)c(F)cc1[C@@H]1c2ccc(O)cc2OC[C@@H]1c1ccccc1] | (S)-3-((S)-3-((2-(2-fluoro-4- ((1S,2S)-6-hydroxy-2-phenyl- 1,2,3,4-tetrahydronaphthalen- 1-yl)-5-methoxyphenyl)-2-aza- spiro[3.5]nonan-7-yl)methyl)- 8-oxo-1,2,3,4,4a,5,8,10-octa- hydro-9H-pyrazino[1′,2′:4,5] [1,4]oxazino[2,3-f]isoindol-9- yl)piperidine-2,6-dione |
| A165* | 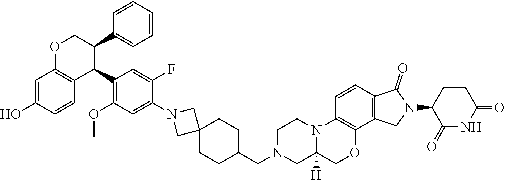 [SMILES: COc1cc(N2CC3(CCC(CN4CCN5c6ccc7c(c6OC[C@H]5C4)CN([C@H]4CCC(=O)NC4=O)C7=O)CC3)C2)c(F)cc1[C@H]1c2ccc(O)cc2OC[C@H]1c1ccccc1] | (S)-3-((R)-7-((2-(2-fluoro-4- ((1R,2R)-6-hydroxy-2-phenyl- 1,2,3,4-tetrahydronaphthalen- 1-yl)-5-methoxyphenyl)-2-aza- spiro[3.5]nonan-7-yl)methyl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A166* | 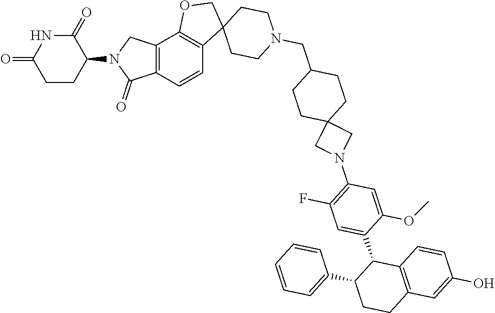 [SMILES: COc1cc(N2CC3(CCC(CN4CCC5(CC4)COc4c5ccc5c4CN([C@H]4CCC(=O)NC4=O)C5=O)CC3)C2)c(F)cc1[C@@H]1c2ccc(O)cc2CC[C@@H]1c1ccccc1] | (S)-3-(1′-((2-(2-fluoro-4-((1S, 2S)-6-hydroxy-2-phenyl- 1,2,3,4-tetrahydronaphthalen- 1-yl)-5-methoxyphenyl)-2-aza- spiro[3.5]nonan-7-yl)methyl)- 6-oxo-6,8-dihydro-2H,7H- spiro[furo[2,3-e]isoindole-3,4′- piperidin]-7-yl)piperidine-2,6- dione |
| A167* | 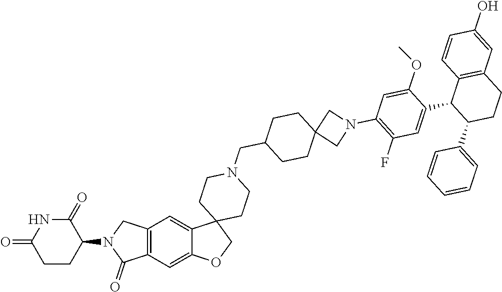 [SMILES: COc1cc(N2CC3(CCC(CN4CCC5(CC4)COc4cc6c(cc45)CN([C@H]4CCC(=O)NC4=O)C6=O)CC3)C2)c(F)cc1[C@@H]1c2ccc(O)cc2CC[C@@H]1c1ccccc1] | (S)-3-(1′-((2-(2-fluoro-4-((1S, 2S)-6-hydroxy-2-phenyl- 1,2,3,4-tetrahydronaphthalen- 1-yl)-5-methoxyphenyl)-2-aza- spiro[3.5]nonan-7-yl)methyl)- 7-oxo-5,7-dihydro-2H,6H-spiro [furo[2,3-f]isoindole-3,4′-piper- idin]-6-yl)piperidine-2,6-dione |
| A168* | 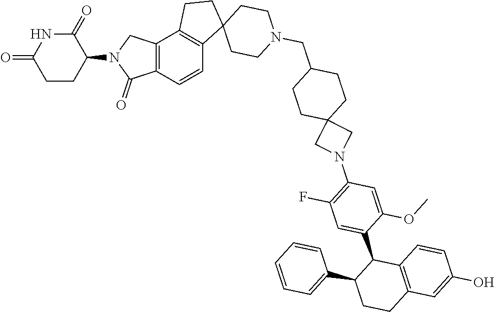 [SMILES: COc1cc(N2CC3(CCC(CN4CCC5(CCc6c5ccc5c6CN([C@H]6CCC(=O)NC6=O)C5=O)CC4)CC3)C2)c(F)cc1[C@H]1c2ccc(O)cc2CC[C@H]1c1ccccc1] | (S)-3-(1′-((2-(2-fluoro-4-((1R, 2R)-6-hydroxy-2-phenyl- 1,2,3,4-tetrahydronaphthalen- 1-yl)-5-methoxyphenyl)-2-aza- spiro[3.5]nonan-7-yl)methyl)- 6-oxo-6,8-dihydro-2H,7H- spiro[furo[2,3-e]isoindole-3,4′- piperidin]-7-yl)piperidine-2,6- dione |
| A169* | 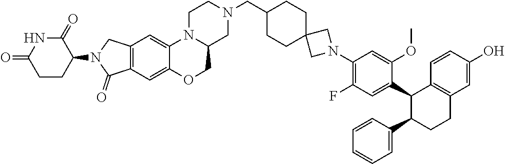 [SMILES: COc1cc(N2CC3(CCC(CN4CCN5c6cc7c(cc6OC[C@@H]5C4)C(=O)N([C@H]4CCC(=O)NC4=O)C7)CC3)C2)c(F)cc1[C@H]1c2ccc(O)cc2CC[C@H]1c1ccccc1] | (S)-3-((S)-3-((2-(2-fluoro-4- ((1R,2R)-6-hydroxy-2-phenyl- 1,2,3,4-tetrahydronaphthalen- 1-yl)-5-methoxyphenyl)-2-aza- spiro[3.5]nonan-7-yl)methyl)- 8-oxo-1,2,3,4,4a,5,8,10-octa- hydro-9H-pyrazino[1′,2′:4,5] [1,4]oxazino[2,3-f]isoindol-9- yl)piperidine-2,6-dione |
| A170* | 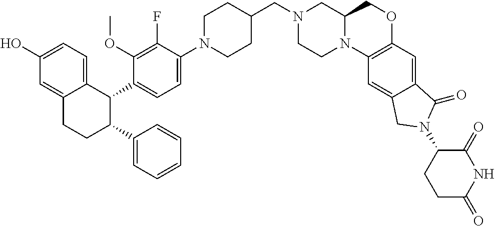 [SMILES: COc1c([C@H]2c3ccc(O)cc3CC[C@H]2c2ccccc2)ccc(N2CCC(CN3CCN4c5cc6c(cc5OC[C@@H]4C3)C(=O)N([C@H]3CCC(=O)NC3=O)C6)CC2)c1F] | (S)-3-((S)-3-((1-(2-fluoro-4- ((1R,2R)-6-hydroxy-2-phenyl- 1,2,3,4-tetrahydronaphthalen- 1-yl)-3-methoxyphenyl)piper- idin-4-yl)methyl)-8-oxo-1,2,3, 4,4a,5,8,10-octahydro-9H- pyrazino[1′,2′:4,5][1,4]oxazino [2,3-f]isoindol-9-yl)piperidine- 2,6-dione |
| A171* | 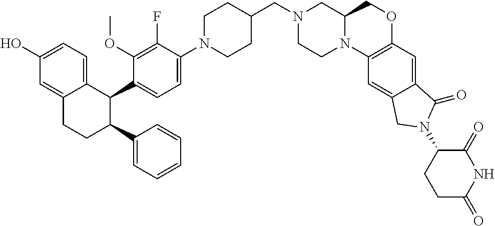 [SMILES: COc1c([C@@H]2c3ccc(O)cc3CC[C@@H]2c2ccccc2)ccc(N2CCC(CN3CCN4c5cc6c(cc5OC[C@@H]4C3)C(=O)N([C@H]3CCC(=O)NC3=O)C6)CC2)c1F] | (S)-3-((S)-3-((1-(2-fluoro-4- ((1S,2S)-6-hydroxy-2-phenyl- 1,2,3,4-tetrahydronaphthalen- 1-yl)-3-methoxyphenyl)piper- idin-4-yl)methyl)-8-oxo-1,2,3, 4,4a,5,8,10-octahydro-9H- pyrazino[1′,2′:4,5][1,4]oxazino [2,3-f]isoindol-9-yl)piperidine- 2,6-dione |
| A172* | 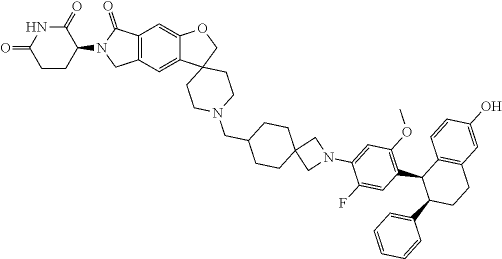 [SMILES: COc1cc(N2CC3(CCC(CN4CCC5(CC4)COc4cc6c(cc45)CN([C@H]4CCC(=O)NC4=O)C6=O)CC3)C2)c(F)cc1[C@H]1c2ccc(O)cc2CC[C@H]1c1ccccc1] | (S)-3-(1′-((2-(2-fluoro-4-((1R, 2R)-6-hydroxy-2-phenyl- 1,2,3,4-tetrahydronaphthalen- 1-yl)-5-methoxyphenyl)-2-aza- spiro[3.5]nonan-7-yl)methyl)- 7-oxo-5,7-dihydro-2H,6H- spiro[furo[2,3-f]isoindole-3,4′- piperidin]-6-yl)piperidine-2,6- dione |
| A173 | 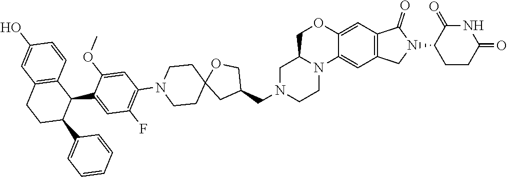 [SMILES: COc1cc(N2CCC3(CC2)C[C@H](CN2CCN4c5cc6c(cc5OC[C@@H]4C2)C(=O)N([C@H]2CCC(=O)NC2=O)C6)CO3)c(F)cc1[C@@H]1c2ccc(O)cc2CC[C@@H]1c1ccccc1] | (S)-3-((S)-3-(((R)-8-(2-fluoro- 4-((1S,2S)-6-hydroxy-2-phen- yl-1,2,3,4-tetrahydronaph- thalen-1-yl)-5-methoxyphenyl)- 1-oxa-8-azaspiro[4.5]decan-3- yl)methyl)-8-oxo-1,2,3,4,4a,5, 8,10-octahydro-9H-pyrazino [1′,2′:4,5][1,4]oxazino[2,3-f] isoindol-9-yl)piperidine-2,6- dione |
| A174 | 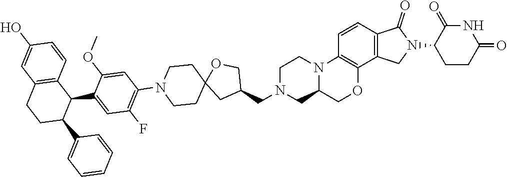 [SMILES: COc1cc(N2CCC3(CC2)C[C@H](CN2CCN4c5ccc6c(c5OC[C@H]4C2)CN([C@H]2CCC(=O)NC2=O)C6=O)CO3)c(F)cc1[C@@H]1c2ccc(O)cc2CC[C@@H]1c1ccccc1] | (S)-3-((R)-7-(((R)-8-(2-fluoro- 4-((1S,2S)-6-hydroxy-2-phen- yl-1,2,3,4-tetrahydronaph- thalen-1-yl)-5-methoxyphenyl)- 1-oxa-8-azaspiro[4.5]decan-3- yl)methyl)-1-oxo-1,3,5,5a,6,7, 8,9-octahydro-2H-pyrazino [1′,2′:4,5][1,4]oxazino[2,3-e] isoindol-2-yl)piperidine-2,6- dione |
| A175 | 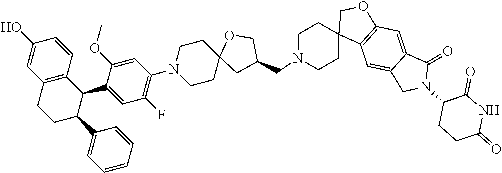 [SMILES: COc1cc(N2CCC3(CC2)C[C@H](CN2CCC4(CC2)COc2cc5c(cc24)CN([C@H]2CCC(=O)NC2=O)C5=O)CO3)c(F)cc1[C@@H]1c2ccc(O)cc2CC[C@@H]1c1ccccc1] | (S)-3-(1′-(((R)-8-(2-fluoro-4- ((1S,2S)-6-hydroxy-2-phenyl- 1,2,3,4-tetrahydronaphthalen-1- yl)-5-methoxyphenyl)-1-oxa- 8-azaspiro[4.5]decan-3-yl)- methyl)-7-oxo-5,7-dihydro-2H, 6H-spiro[furo[2,3-f]isoindole- 3,4′-piperidin]-6-yl)-piperidine- 2,6-dione |
| A176 | 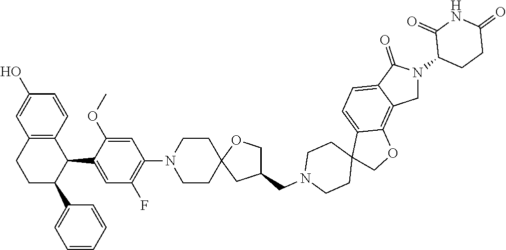 [SMILES: COc1cc(N2CCC3(CC2)C[C@H](CN2CCC4(CC2)COc2c4ccc4c2CN([C@H]2CCC(=O)NC2=O)C4=O)CO3)c(F)cc1[C@@H]1c2ccc(O)cc2CC[C@@H]1c1ccccc1] | (S)-3-(1′-(((R)-8-(2-fluoro-4- ((1S,2S)-6-hydroxy-2-phenyl- 1,2,3,4-tetrahydronaphthalen- 1-yl)-5-methoxyphenyl)-1-oxa- 8-azaspiro[4.5]decan-3-yl)- methyl)-6-oxo-6,8-dihydro-2H, 7H-spiro[furo[2,3-c]isoindole- 3,4′-piperidin]-7-yl)piperidine- 2,6-dione |
| A177 | 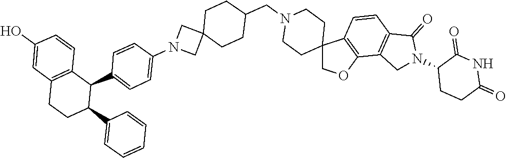 [SMILES: O=C1CC[C@H](N2Cc3c(ccc4c3OCC43CCN(CC4CCC5(CC4)CN(c4ccc([C@@H]6c7ccc(O)cc7CC[C@@H]6c6ccccc6)cc4)C5)CC3)C2=O)C(=O)N1] | (S)-3-(1′-((2-(4-((1R,2S)-6- hydroxy-2-phenyl-1,2,3,4-tetra- hydronaphthalen-1-yl)phenyl)- 2-azaspiro[3.5]nonan-7-yl)- methyl)-6-oxo-6,8-dihydro- 2H,7H-spiro[furo[2,3-e]iso- indole-3,4′-piperidin]-7-yl)- piperidine-2,6-dione |
| A178 | 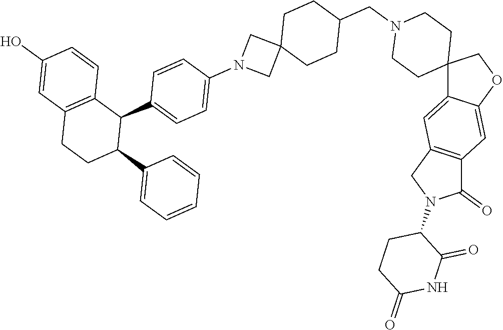 [SMILES: O=C1CC[C@H](N2Cc3cc4c(cc3C2=O)OCC42CCN(CC3CCC4(CC3)CN(c3ccc([C@@H]5c6ccc(O)cc6CC[C@@H]5c5ccccc5)cc3)C4)CC2)C(=O)N1] | (S)-3-(1′-((2-(4-((1R,2S)-6- hydroxy-2-phenyl-1,2,3,4-tetra- hydronaphthalen-1-yl)phenyl)- 2-azaspiro[3.5]nonan-7-yl)- methyl)-7-oxo-5,7-dihydro-2H, 6H-spiro[furo[2,3-f]isoindole- 3,4′-piperidin]-6-yl)piperidine- 2,6-dione |
| A179 | 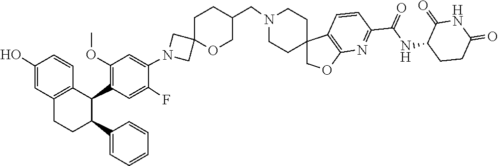 [SMILES: COc1cc(N2CC3(CCC(CN4CCC5(CC4)COc4nc(C(=O)N[C@H]6CCC(=O)NC6=O)ccc45)CO3)C2)c(F)cc1[C@@H]1c2ccc(O)cc2CC[C@@H]1c1ccccc1] | N-((S)-2,6-dioxopiperidin-3- yl)-1′-((2-(2-fluoro-4-((1S,2S)- 6-hydroxy-2-phenyl-1,2,3,4- tetrahydronaphthalen-1-yl)-5- methoxyphenyl)-2-azaspiro [3.5]nonan-7-yl)methyl)-2H- spiro[furo[2,3-b]pyridine-3,4′- piperidine]-6-carboxamide |
| A180 | 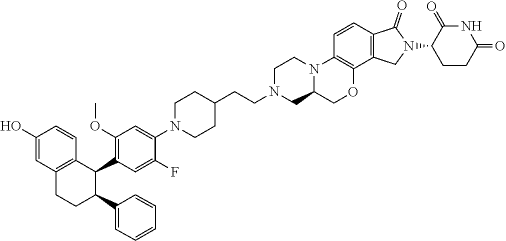 [SMILES: COc1cc(N2CCC(CCN3CCN4c5ccc6c(c5OC[C@H]4C3)CN([C@H]3CCC(=O)NC3=O)C6=O)CC2)c(F)cc1[C@@H]1c2ccc(O)cc2CC[C@@H]1c1ccccc1] | (S)-3-((R)-7-(2-(1-(2-fluoro-4- ((1S,2S)-6-hydroxy-2-phenyl- 1,2,3,4-tetrahydronaphthalen- 1-yl)-5-methoxyphenyl)piper- idin-4-yl)ethyl)-1-oxo-1,3,5,5a, 6,7,8,9-octahydro-2H-pyrazino [1′,2′:4,5][1,4]oxazino[2,3-e] isoindol-2-yl)piperidine-2,6- dione |
| A181 | 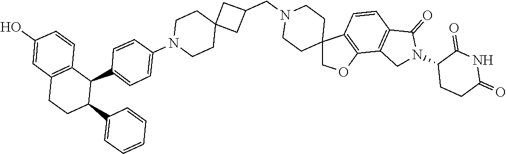 [SMILES: O=C1CC[C@H](N2Cc3c(ccc4c3OCC43CCN(CC4CC5(CCN(c6ccc([C@@H]7c8ccc(O)cc8CC[C@@H]7c7ccccc7)cc6)CC5)C4)CC3)C2=O)C(=O)N1] | (S)-3-(1′-((7-(4-((1R,2S)-6- hydroxy-2-phenyl-1,2,3,4-tetra- hydronaphthalen-1-yl)phenyl)- 7-azaspiro[3.5]nonan-2-yl)- methyl)-6-oxo-6,8-dihydro-2H, 7H-spiro[furo[2,3-e]isoindole- 3,4′-piperidin]-7-yl)piperidine- 2,6-dione |
| A182 | 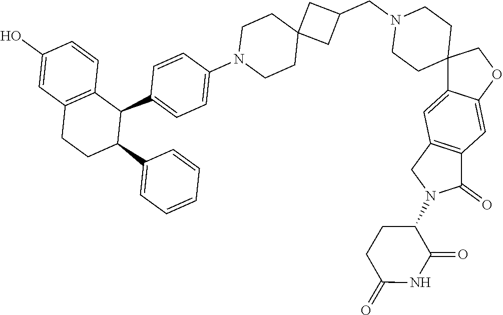 [SMILES: O=C1CC[C@H](N2Cc3cc4c(cc3C2=O)OCC42CCN(CC3CC4(CCN(c5ccc([C@@H]6c7ccc(O)cc7CC[C@@H]6c6ccccc6)cc5)CC4)C3)CC2)C(=O)N1] | (S)-3-(1′-((7-(4-((1R,2S)-6- hydroxy-2-phenyl-1,2,3,4-tetra- hydronaphthalen-1-yl)phenyl)- 7-azaspiro[3.5]nonan-2-yl)- methyl)-7-oxo-5,7-dihydro-2H, 6H-spiro[furo[2,3-f]isoindole- 3,4′-piperidin]-6-yl)piperidine- 2,6-dione |
| A183 | 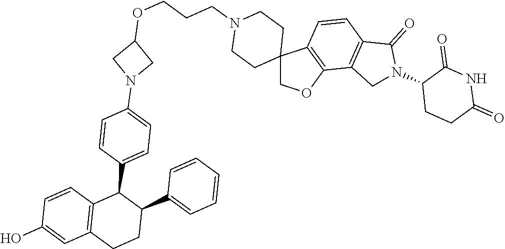 [SMILES: O=C1CC[C@H](N2Cc3c(ccc4c3OCC43CCN(CCCOC4CN(c5ccc([C@@H]6c7ccc(O)cc7CC[C@@H]6c6ccccc6)cc5)C4)CC3)C2=O)C(=O)N1] | (S)-3-(1′-(3-((1-(4-((1R,2S)-6- hydroxy-2-phenyl-1,2,3,4-tetra- hydronaphthalen-1-yl)phenyl)- azetidin-3-yl)oxy)propyl)-6- oxo-6,8-dihydro-2H,7H-spiro [furo[2,3-e]isoindole-3,4′- piperidin]-7-yl)piperidine-2,6- dione |
| A184 | 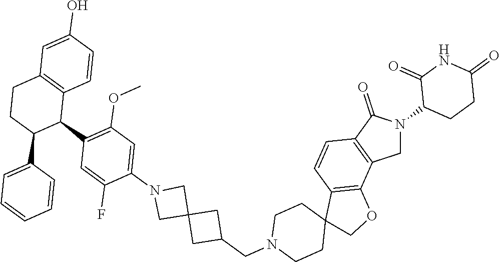 | (S)-3-(1′-((2-(2-fluoro-4-((1S, 2S)-6-hydroxy-2-phenyl- 1,2,3,4-tetrahydronaphthalen- 1-yl)-5-methoxyphenyl)-2-aza- spiro[3.3]heptan-6-yl)methyl)- 6-oxo-6,8-dihydro-2H,7H- spiro[furo[2,3-e]isoindole-3,4′- piperidin]-7-yl)piperidine-2,6- dione |
| A185 | 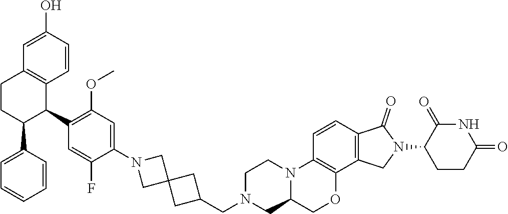 [SMILES: COc1cc(N2CC3(CC(CN4CCN5c6ccc7c(c6OC[C@H]5C4)CN([C@H]4CCC(=O)NC4=O)C7=O)C3)C2)c(F)cc1[C@@H]1c2ccc(O)cc2CC[C@@H]1c1ccccc1] | (S)-3-((R)-7-((2-(2-fluoro-4- ((1S,2S)-6-hydroxy-2-phenyl- 1,2,3,4-tetrahydronaphthalen-1- yl)-5-methoxyphenyl)-2-aza- spiro[3.3]heptan-6-yl)methyl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A186 | 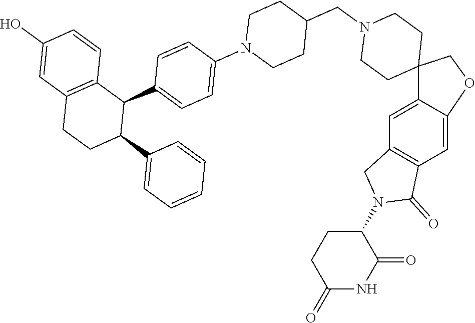 [SMILES: O=C1CC[C@H](N2Cc3cc4c(cc3C2=O)OCC42CCN(CC3CCN(c4ccc([C@@H]5c6ccc(O)cc6CC[C@@H]5c5ccccc5)cc4)CC3)CC2)C(=O)N1] | (S)-3-(1′-((1-(4-((1R,2S)-6- hydroxy-2-phenyl-1,2,3,4-tetra- hydronaphthalen-1-yl)phenyl)- piperidin-4-yl)methyl)-7-oxo- 5,7-dihydro-2H,6H-spiro[furo [2,3-f]isoindole-3,4′-piperidin]- 6-yl)piperidine-2,6-dione |
| A187 | 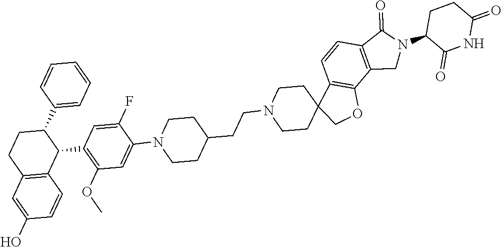 [SMILES: COc1cc(N2CCC(CCN3CCC4(CC3)COc3c4ccc4c3CN([C@H]3CCC(=O)NC3=O)C4=O)CC2)c(F)cc1[C@@H]1c2ccc(O)cc2CC[C@@H]1c1ccccc1] | (S)-3-(1′-(2-(1-(2-fluoro-4- ((1S,2S)-6-hydroxy-2-phenyl- 1,2,3,4-tetrahydronaphthalen-1- yl)-5-methoxyphenyl)piperidin- 4-yl)ethyl)-6-oxo-6,8-dihydro- 2H,7H-spiro[furo[2,3-e]iso- indole-3,4′-piperidin]-7-yl)- piperidine-2,6-dione |
| A188 | 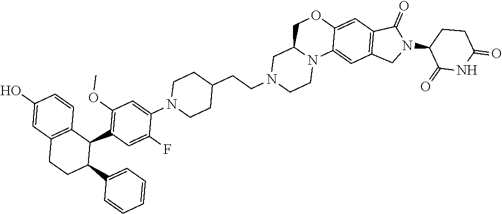 [SMILES: COc1cc(N2CCC(CCN3CCN4c5cc6c(cc5OC[C@@H]4C3)C(=O)N([C@H]3CCC(=O)NC3=O)C6)CC2)c(F)cc1[C@@H]1c2ccc(O)cc2CC[C@@H]1c1ccccc1] | (S)-3-((S)-3-(2-(1-(2-fluoro-4- ((1S,2S)-6-hydroxy-2-phenyl- 1,2,3,4-tetrahydronaphthalen- 1-yl)-5-methoxyphenyl)piper- idin-4-yl)ethyl)-8-oxo-1,2,3,4, 4a,5,8,10-octahydro-9H-pyr- azino[1′,2′:4,5][1,4]oxazino [2,3-f]isoindol-9-yl)piperidine- 2,6-dione |
| A189 | 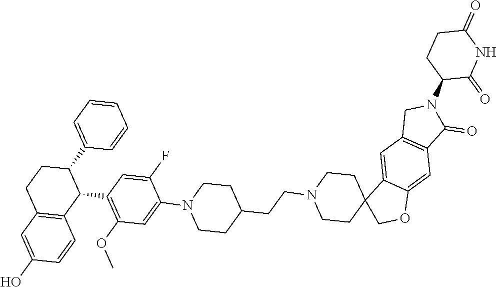 [SMILES: COc1cc(N2CCC(CCN3CCC4(CC3)COc3cc5c(cc34)CN([C@H]3CCC(=O)NC3=O)C5=O)CC2)c(F)cc1[C@@H]1c2ccc(O)cc2CC[C@@H]1c1ccccc1] | (S)-3-(1′-(2-(1-(2-fluoro-4- ((1S,2S)-6-hydroxy-2-phenyl- 1,2,3,4-tetrahydronaphthalen- 1-yl)-5-methoxyphenyl)piper- idin-4-yl)ethyl)-7-oxo-5,7- dihydro-2H,6H-spiro[furo[2,3- f]isoindole-3,4′-piperidin]-6- yl)piperidine-2,6-dione |
| A190* | 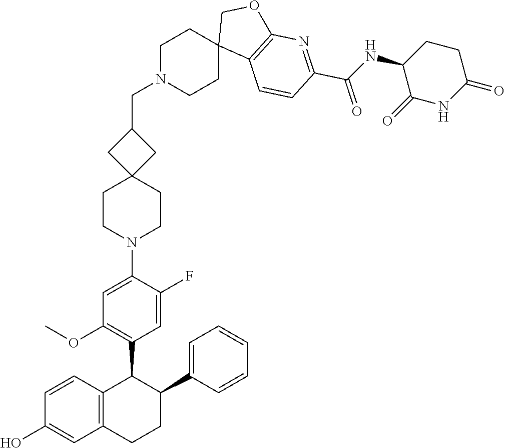 [SMILES: COc1cc(N2CCC3(CC2)CC(CN2CCC4(CC2)COc2nc(C(=O)N[C@H]5CCC(=O)NC5=O)ccc24)C3)c(F)cc1[C@@H]1c2ccc(O)cc2CC[C@@H]1c1ccccc1] | N-((S)-2,6-dioxopiperidin-3- yl)-1′-((7-(2-fluoro-4-((1S,2S)- 6-hydroxy-2-phenyl-1,2,3,4- tetrahydronaphthalen-1-yl)-5- methoxyphenyl)-7-azaspiro [3.5]nonan-2-yl)methyl)-2H- spiro[furo[2,3-b]pyridine-3,4′- piperidine]-6-carboxamide |
| A191 | 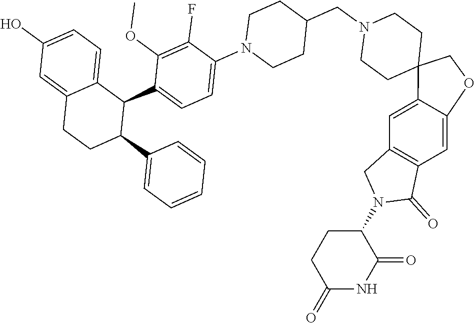 [SMILES: COc1c([C@@H]2c3ccc(O)cc3CC[C@@H]2c2ccccc2)ccc(N2CCC(CN3CCC4(CC3)COc3cc5c(cc34)CN([C@H]3CCC(=O)NC3=O)C5=O)CC2)c1F] | (S)-3-(1′-((1-(2-fluoro-4-((1S, 2S)-6-hydroxy-2-phenyl- 1,2,3,4-tetrahydronaphthalen- 1-yl)-3-methoxyphenyl)piper- idin-4-yl)methyl)-7-oxo-5,7- dihydro-2H,6H-spiro[furo[2,3- f]isoindole-3,4′-piperidin]-6- yl)piperidine-2,6-dione |
| A192 | 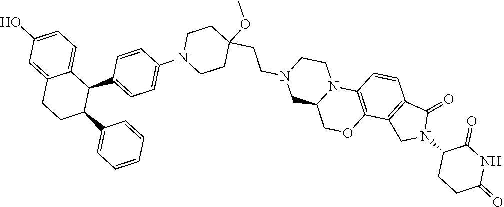 [SMILES: COC1(CCN2CCN3c4ccc5c(c4OC[C@H]3C2)CN([C@H]2CCC(=O)NC2=O)C5=O)CCN(c2ccc([C@@H]3c4ccc(O)cc4CC[C@@H]3c3ccccc3)cc2)CC1] | (S)-3-((R)-7-(2-(1-(4-((1R,2S)- 6-hydroxy-2-phenyl-1,2,3,4- tetrahydronaphthalen-1-yl)- phenyl)-4-methoxypiperidin-4- yl)cthyl)-1-oxo-1,3,5,5a,6,7,8, 9-octahydro-2H-pyrazino[1′,2′: 4,5][1,4]oxazino[2,3-e]isoindol- 2-yl)piperidine-2,6-dione |
| A193 | 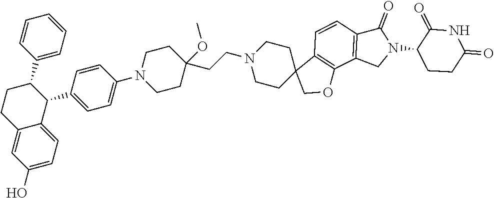 [SMILES: COC1(CCN2CCC3(CC2)COc2c3ccc3c2CN([C@H]2CCC(=O)NC2=O)C3=O)CCN(c2ccc([C@@H]3c4ccc(O)cc4CC[C@@H]3c3ccccc3)cc2)CC1] | (S)-3-(1′-(2-(1-(4-((1R,2S)-6- hydroxy-2-phenyl-1,2,3,4-tetra- hydronaphthalen-1-yl)phenyl)- 4-methoxypiperidin-4-yl)- ethyl)-6-oxo-6,8-dihydro-2H, 7H-spiro[furo[2,3-e]isoindole- 3,4′-piperidin]-7-yl)piperidine- 2,6-dione |
| A194 | 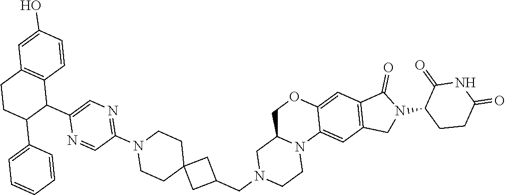 [SMILES: O=C1CC[C@H](N2Cc3cc4c(cc3C2=O)OC[C@@H]2CN(CC3CC5(CCN(c6cnc(C7c8ccc(O)cc8CCC7c7ccccc7)cn6)CC5)C3)CCN42)C(=O)N1] | (3S)-3-((4aS)-3-((7-(5-(6- hydroxy-2-phenyl-1,2,3,4-tetra- hydronaphthalen-1-yl)pyrazin- 2-yl)-7-azaspiro[3.5]nonan-2- yl)methyl)-8-oxo-1,2,3,4,4a,5, 8,10-octahydro-9H-pyrazino [1′,2′:4,5][1,4]oxazino[2,3-f] isoindol-9-yl)piperidine-2,6- dione |
| A195 | 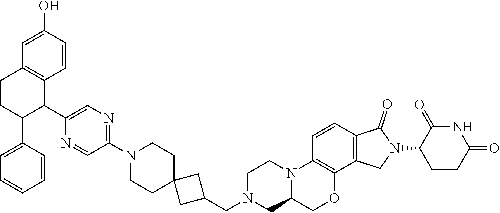 [SMILES: O=C1CC[C@H](N2Cc3c(ccc4c3OC[C@H]3CN(CC5CC6(CCN(c7cnc(C8c9ccc(O)cc9CCC8c8ccccc8)cn7)CC6)C5)CCN43)C2=O)C(=O)N1] | (3S)-3-((5aR)-7-((7-(5-(6- hydroxy-2-phenyl-1,2,3,4-tetra- hydronaphthalen-1-yl)pyrazin- 2-yl)-7-azaspiro[3.5]nonan-2- yl)methyl)-1-oxo-1,3,5,5a,6,7, 8,9-octahydro-2H-pyrazino [1′,2′:4,5][1,4]oxazino[2,3-e] isoindol-2-yl)piperidine-2,6- dione |
| A196 | 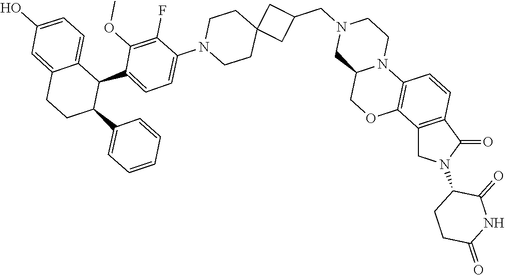 [SMILES: COc1c([C@@H]2c3ccc(O)cc3CC[C@@H]2c2ccccc2)ccc(N2CCC3(CC2)CC(CN2CCN4c5ccc6c(c5OC[C@H]4C2)CN([C@H]2CCC(=O)NC2=O)C6=O)C3)c1F] | (S)-3-((R)-7-((7-(2-fluoro-4- ((1S,2S)-6-hydroxy-2-phenyl- 1,2,3,4-tetrahydronaphthalen- 1-yl)-3-methoxyphenyl)-7-aza- spiro[3.5]nonan-2-yl)methyl)- 1-oxo-1,3,5,5a,6,7,8,9-octa- hydro-2H-pyrazino[1′,2′:4,5] [1,4]oxazino[2,3-e]isoindol-2- yl)piperidine-2,6-dione |
| A197 | 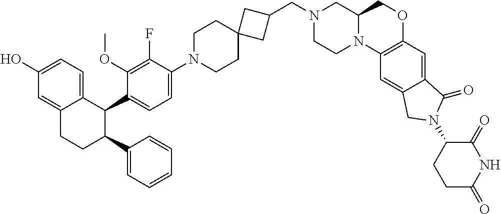 [SMILES: COc1c([C@@H]2c3ccc(O)cc3CC[C@@H]2c2ccccc2)ccc(N2CCC3(CC2)CC(CN2CCN4c5cc6c(cc5OC[C@@H]4C2)C(=O)N([C@H]2CCC(=O)NC2=O)C6)C3)c1F] | (S)-3-((S)-3-((7-(2-fluoro-4- ((1S,2S)-6-hydroxy-2-phenyl- 1,2,3,4-tetrahydronaphthalen- 1-yl)-3-methoxyphenyl)-7-aza- spiro[3.5]nonan-2-yl)methyl)- 8-oxo-1,2,3,4,4a,5,8,10-octa- hydro-9H-pyrazino[1′,2′:4,5] [1,4]oxazino[2,3-f]isoindol-9- yl)piperidine-2,6-dione |
| A198 | 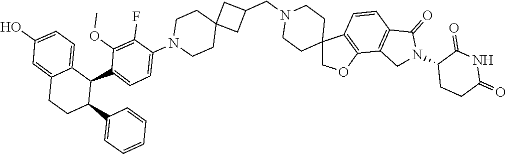 [SMILES: COc1c([C@@H]2c3ccc(O)cc3CC[C@@H]2c2ccccc2)ccc(N2CCC3(CC2)CC(CN2CCC4(CC2)COc2c4ccc4c2CN([C@H]2CCC(=O)NC2=O)C4=O)C3)c1F] | (S)-3-(1′-((7-(2-fluoro-4-((1S, 2S)-6-hydroxy-2-phenyl- 1,2,3,4-tetrahydronaphthalen- 1-yl)-3-methoxyphenyl)-7-aza- spiro[3.5]nonan-2-yl)methyl)- 6-oxo-6,8-dihydro-2H,7H- spiro[furo[2,3-e]isoindole-3,4′- piperidin]-7-yl)piperidine-2,6- dione |
| A199 | 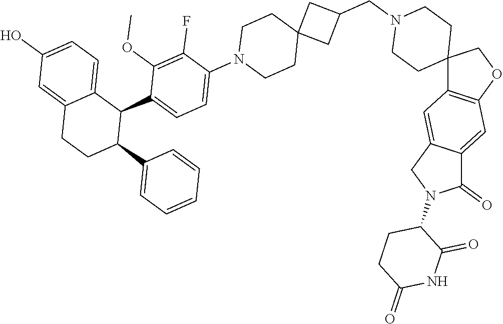 [SMILES: COc1c([C@@H]2c3ccc(O)cc3CC[C@@H]2c2ccccc2)ccc(N2CCC3(CC2)CC(CN2CCC4(CC2)COc2cc5c(cc24)CN([C@H]2CCC(=O)NC2=O)C5=O)C3)c1F] | (S)-3-(1′-((7-(2-fluoro-4-((1S, 2S)-6-hydroxy-2-phenyl- 1,2,3,4-tetrahydronaphthalen- 1-yl)-3-methoxyphenyl)-7-aza- spiro[3.5]nonan-2-yl)methyl)- 7-oxo-5,7-dihydro-2H,6H- spiro[furo[2,3-f]isoindole-3,4′- piperidin]-6-yl)piperidine-2,6- dione |
| A200 | 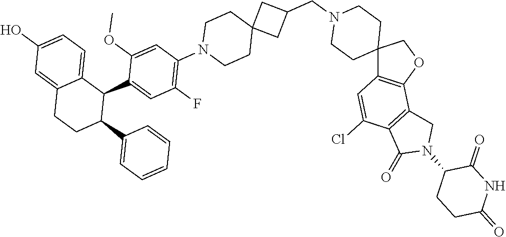 [SMILES: COc1cc(N2CCC3(CC2)CC(CN2CCC4(CC2)COc2c4cc(Cl)c4c2CN([C@H]2CCC(=O)NC2=O)C4=O)C3)c(F)cc1[C@@H]1c2ccc(O)cc2CC[C@@H]1c1ccccc1] | (S)-3-(5-chloro-1′-((7-(2- fluoro-4-((1S,2S)-6-hydroxy- 2-phenyl-1,2,3,4-tetrahydro- naphthalen-1-yl)-5-methoxy- phenyl)-7-azaspiro[3.5]nonan- 2-yl)methyl)-6-oxo-6,8- dihydro-2H,7H-spiro[furo[2,3- e]isoindole-3,4′-piperidin]-7- yl)piperidine-2,6-dione |
| A201 | 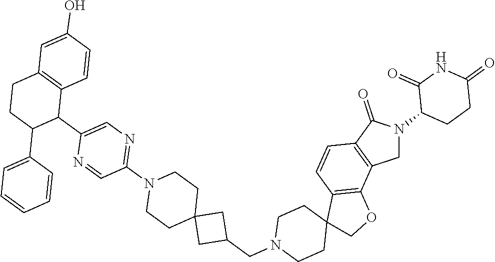 [SMILES: O=C1CC[C@H](N2Cc3c(ccc4c3OCC43CCN(CC4CC5(CCN(c6cnc(C7c8ccc(O)cc8CCC7c7ccccc7)cn6)CC5)C4)CC3)C2=O)C(=O)N1] | (3S)-3-(1′-((7-(5-(6-hydroxy- 2-phenyl-1,2,3,4-tetrahydro- naphthalen-1-yl)pyrazin-2-yl)- 7-azaspiro[3.5]nonan-2-yl)- methyl)-6-oxo-6,8-dihydro-2H, 7H-spiro[furo[2,3-e]isoindole- 3,4′-piperidin]-7-yl)piperidine- 2,6-dione |
| A202 | 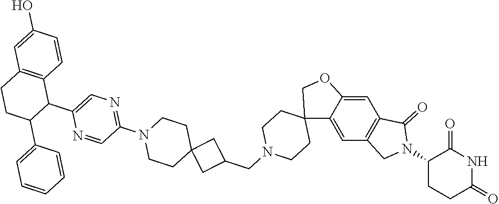 [SMILES: O=C1CC[C@H](N2Cc3cc4c(cc3C2=O)OCC42CCN(CC3CC4(CCN(c5cnc(C6c7ccc(O)cc7CCC6c6ccccc6)cn5)CC4)C3)CC2)C(=O)N1] | (3S)-3-(1′-((7-(5-(6-hydroxy-2- phenyl-1,2,3,4-tetrahydronaph- thalen-1-yl)pyrazin-2-yl)-7- azaspiro[3.5]nonan-2-yl)meth- yl)-7-oxo-5,7-dihydro-2H,6H- spiro[furo[2,3-f]isoindole-3,4′- piperidin]-6-yl)piperidine-2,6- dione |
| A203 | 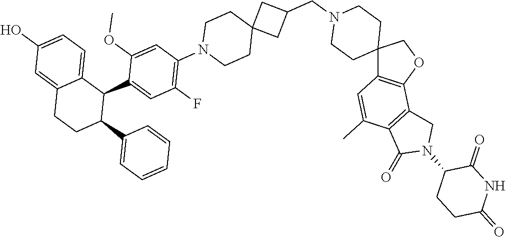 [SMILES: COc1cc(N2CCC3(CC2)CC(CN2CCC4(CC2)COc2c4cc(C)c4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4-((1S, 2S)-6-hydroxy-2-phenyl- 1,2,3,4-tetrahydronaphthalen- 1-yl)-5-methoxyphenyl)-7-aza- spiro[3.5]nonan-2-yl)methyl)- 5-methyl-6-oxo-6,8-dihydro- 2H,7H-spiro[furo[2,3-e]iso- indole-3,4′-piperidin]-7-yl)- piperidine-2,6-dione |
| A204 | 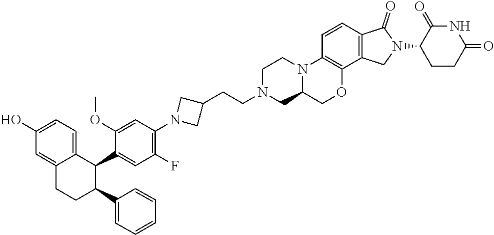 [SMILES: COc1cc(N2CC(CCN3CCN4c5ccc6c(c5OC[C@H]4C3)CN([C@H]3CCC(=O)NC3=O)C6=O)C2)c(F)cc1[C@@H]1c2ccc(O)cc2CC[C@@H]1c1ccccc1] | (S)-3-((R)-7-(2-(1-(2-fluoro-4- ((1S,2S)-6-hydroxy-2-phenyl- 1,2,3,4-tetrahydronaphthalen- 1-yl)-5-methoxyphenyl)azet- idin-3-yl)ethyl)-1-oxo-1,3,5,5a, 6,7,8,9-octahydro-2H-pyrazino [1′,2′:4,5][1,4]oxazino[2,3-e] isoindol-2-yl)piperidine-2,6- dione |
| A205 | 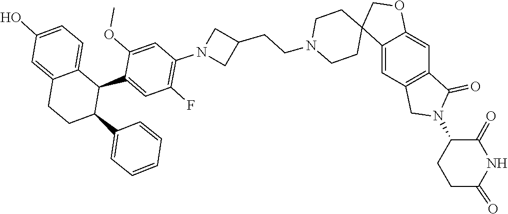 [SMILES: COc1cc(N2CC(CCN3CCC4(CC3)COc3cc5c(cc34)CN([C@H]3CCC(=O)NC3=O)C5=O)C2)c(F)cc1[C@@H]1c2ccc(O)cc2CC[C@@H]1c1ccccc1] | (S)-3-(1′-(2-(1-(2-fluoro-4- ((1S,2S)-6-hydroxy-2-phenyl- 1,2,3,4-tetrahydronaphthalen- 1-yl)-5-methoxyphenyl)-azet- idin-3-yl)ethyl)-7-oxo-5,7- dihydro-2H,6H-spiro[furo[2,3- f]isoindole-3,4′-piperidin]-6- yl)piperidine-2,6-dione |
| A206 | 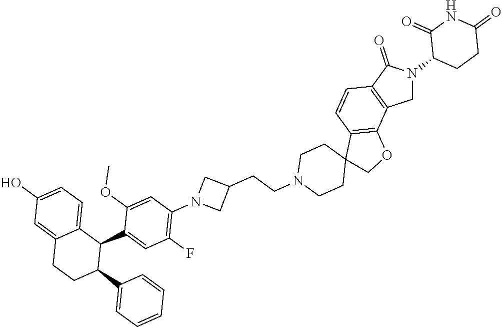 [SMILES: COc1cc(N2CC(CCN3CCC4(CC3)COc3c4ccc4c3CN([C@H]3CCC(=O)NC3=O)C4=O)C2)c(F)cc1[C@@H]1c2ccc(O)cc2CC[C@@H]1c1ccccc1] | (S)-3-(1′-(2-(1-(2-fluoro-4- ((1S,2S)-6-hydroxy-2-phenyl- 1,2,3,4-tetrahydronaphthalen- 1-yl)-5-methoxyphenyl)azet- idin-3-yl)ethyl)-6-oxo-6,8- dihydro-2H,7H-spiro[furo[2,3- e]isoindole-3,4′-piperidin]-7- yl)piperidine-2,6-dione |
| A208 | 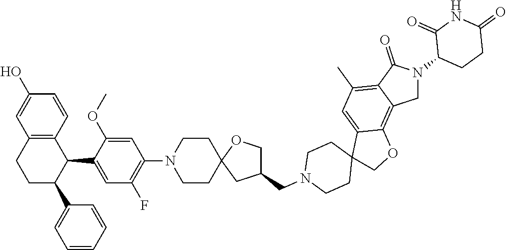 [SMILES: COc1cc(N2CCC3(CC2)C[C@H](CN2CCC4(CC2)COc2c4cc(C)c4c2CN([C@H]2CCC(=O)NC2=O)C4=O)CO3)c(F)cc1[C@@H]1c2ccc(O)cc2CC[C@@H]1c1ccccc1] | (3S)-3-[1′-[(3R)-8-[2-fluoro-4- [(1S,2S)-6-hydroxy-2-phenyl- tetralin-1-yl]-5-methoxy-phen- yl]-1-oxa-8-azaspiro[4.5]decan- 3-yl]methyl]-5-methyl-6-oxo- spiro[2,8-dihydrofuro[2,3-e] isoindole-3,4′-piperidine]-7- yl]piperidine-2,6-dione |
| A209 | 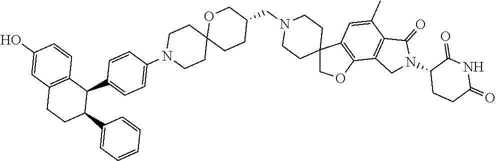 [SMILES: Cc1cc2c(c3c1C(=O)N([C@H]1CCC(=O)NC1=O)C3)OCC21CCN(C[C@@H]2CCC3(CCN(c4ccc([C@@H]5c6ccc(O)cc6CC[C@@H]5c5ccccc5)cc4)CC3)OC2)CC1] | (S)-3-(1′-(((S)-9-(4-((1R,2S)-6- hydroxy-2-phenyl-1,2,3,4-tetra- hydronaphthalen-1-yl)phenyl)- 1-oxa-9-azaspiro[5.5]undecan- 3-yl)methyl)-5-mcthyl-6-oxo- 6,8-dihydro-2H,7H-spiro[furo [2,3-e]isoindole-3,4′-piperidin]- 7-yl)piperidine-2,6-dione |
| A210 | 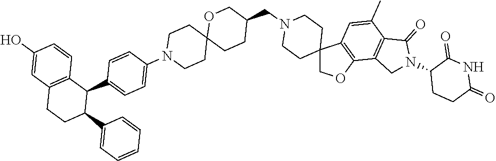 [SMILES: Cc1cc2c(c3c1C(=O)N([C@H]1CCC(=O)NC1=O)C3)OCC21CCN(C[C@H]2CCC3(CCN(c4ccc([C@@H]5c6ccc(O)cc6CC[C@@H]5c5ccccc5)cc4)CC3)OC2)CC1] | (S)-3-(1′-(((R)-9-(4-((1R,2S)- 6-hydroxy-2-phenyl-1,2,3,4- tetrahydronaphthalen-1-yl)- phenyl)-1-oxa-9-azaspiro[5.5] undecan-3-yl)methyl)-5-meth- yl-6-oxo-6,8-dihydro-2H,7H- spiro[furo[2,3-e]isoindole-3,4′- piperidin]-7-yl)piperidine-2,6- dione |
| A211 | 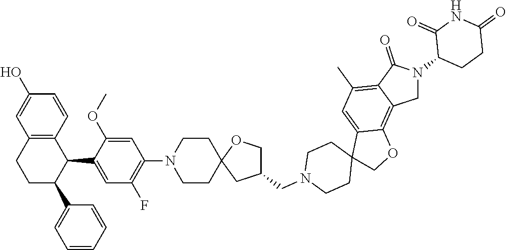 [SMILES: COc1cc(N2CCC3(CC2)C[C@@H](CN2CCC4(CC2)COc2c4cc(C)c4c2CN([C@H]2CCC(=O)NC2=O)C4=O)CO3)c(F)cc1[C@@H]1c2ccc(O)cc2CC[C@@H]1c1ccccc1] | (3S)-3-[1′-[[(3S)-8-[2-fluoro-4- [(1S,2S)-6-hydroxy-2-phenyl- tetralin-1-yl]-5-methoxy-phen- yl]-1-oxa-8-azaspiro[4.5]decan- 3-yl]methyl]-5-methyl-6-oxo- spiro[2,8-dihydrofuro[2,3-e] isoindole-3,4′-piperidine]-7- yl]piperidine-2,6-dione |
| A212 | 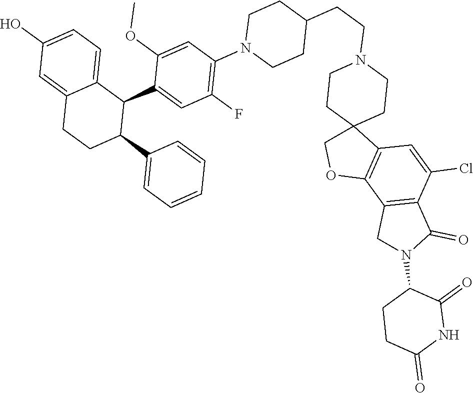 [SMILES: COc1cc(N2CCC(CCN3CCC4(CC3)COc3c4cc(Cl)c4c3CN([C@H]3CCC(=O)NC3=O)C4=O)CC2)c(F)cc1[C@@H]1c2ccc(O)cc2CC[C@@H]1c1ccccc1] | (S)-3-(5-chloro-1′-(2-(1-(2- fluoro-4-((1S,2S)-6-hydroxy- 2-phenyl-1,2,3,4-tetrahydro- naphthalen-1-yl)-5-methoxy- phenyl)piperidin-4-yl)ethyl)- 6-oxo-6,8-dihydro-2H,7H- spiro[furo[2,3-e]isoindole-3,4′- piperidin]-7-yl)piperidine-2,6- dione |
| A213 | 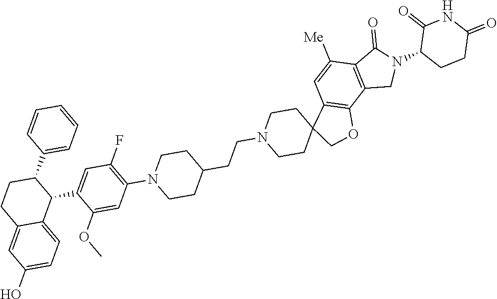 [SMILES: COc1cc(N2CCC(CCN3CCC4(CC3)COc3c4cc(C)c4c3CN([C@H]3CCC(=O)NC3=O)C4=O)CC2)c(F)cc1[C@@H]1c2ccc(O)cc2CC[C@@H]1c1ccccc1] | (S)-3-(1′-(2-(1-(2-fluoro-4- ((1S,2S)-6-hydroxy-2-phenyl- 1,2,3,4-tetrahydronaphthalen- 1-yl)-5-methoxyphenyl)piper- idin-4-yl)ethyl)-5-methyl-6- oxo-6,8-dihydro-2H,7H-spiro [furo[2,3-e]isoindole-3,4′- piperidin]-7-yl)piperidine-2,6- dione |
| A214 | 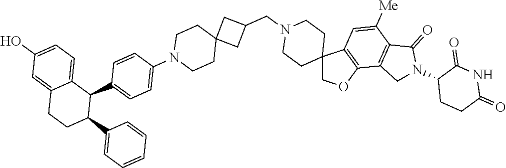 [SMILES: Cc1cc2c(c3c1C(=O)N([C@H]1CCC(=O)NC1=O)C3)OCC21CCN(CC2CC3(CCN(c4ccc([C@@H]5c6ccc(O)cc6CC[C@@H]5c5ccccc5)cc4)CC3)C2)CC1] | (S)-3-(1′-((7-(4-((1R,2S)-6- hydroxy-2-phenyl-1,2,3,4-tetra- hydronaphthalen-1-yl)phenyl)- 7-azaspiro[3.5]nonan-2-yl)- methyl)-5-methyl-6-oxo-6,8- dihydro-2H,7H-spiro[furo[2,3- c]isoindolc-3,4′-pipcridin]-7- yl)piperidine-2,6-dione |
| A216 | 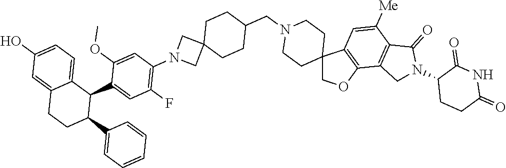 [SMILES: COc1cc(N2CC3(CCC(CN4CCC5(CC4)COc4c5cc(C)c5c4CN([C@H]4CCC(=O)NC4=O)C5=O)CC3)C2)c(F)cc1[C@@H]1c2ccc(O)cc2CC[C@@H]1c1ccccc1] | (S)-3-(1′-((2-(2-fluoro-4-((1S, 2S)-6-hydroxy-2-phenyl- 1,2,3,4-tetrahydronaphthalen- 1-yl)-5-methoxyphenyl)-2-aza- spiro[3.5]nonan-7-yl)methyl)- 5-methyl-6-oxo-6,8-dihydro- 2H,7H-spiro[furo[2,3-e]iso- indole-3,4′-piperidin]-7-yl)- piperidine-2,6-dione |
| A217 | 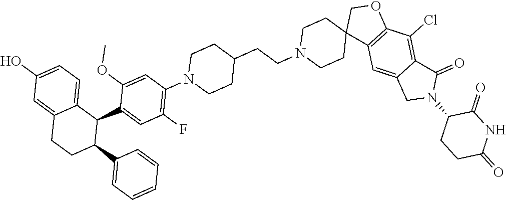 [SMILES: COc1cc(N2CCC(CCN3CCC4(CC3)COc3c4cc4c(c3Cl)C(=O)N([C@H]3CCC(=O)NC3=O)C4)CC2)c(F)cc1[C@@H]1c2ccc(O)cc2CC[C@@H]1c1ccccc1] | (S)-3-(8-chloro-1′-(2-(1-(2- fluoro-4-((1S,2S)-6-hydroxy-2- phenyl-1,2,3,4-tetrahydronaph- thalen-1-yl)-5-methoxyphenyl)- piperidin-4-yl)ethyl)-7-oxo-5,7- dihydro-2H,6H-spiro[furo[2,3- f]isoindole-3,4′-piperidin]-6- yl)piperidine-2,6-dione |
| A218 | 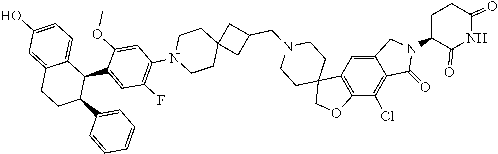 [SMILES: COc1cc(N2CCC3(CC2)CC(CN2CCC4(CC2)COc2c4cc4c(c2Cl)C(=O)N([C@H]2CCC(=O)NC2=O)C4)C3)c(F)cc1[C@@H]1c2ccc(O)cc2CC[C@@H]1c1ccccc1] | (S)-3-(8-chloro-1′-((7-(2- fluoro-4-((1S,2S)-6-hydroxy- 2-phenyl-1,2,3,4-tetrahydro- naphthalen-1-yl)-5-methoxy- phenyl)-7-azaspiro[3.5]nonan- 2-yl)methyl)-7-oxo-5,7- dihydro-2H,6H-spiro[furo[2,3- f]isoindole-3,4′-piperidin]-6- yl)piperidine-2,6-dione |
| A219 | 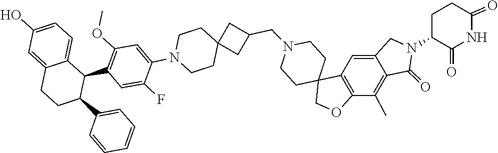 [SMILES: COc1cc(N2CCC3(CC2)CC(CN2CCC4(CC2)COc2c4cc4c(c2C)C(=O)N([C@@H]2CCC(=O)NC2=O)C4)C3)c(F)cc1[C@@H]1c2ccc(O)cc2CC[C@@H]1c1ccccc1] | (R)-3-(1′-((7-(2-fluoro-4-((1S, 2S)-6-hydroxy-2-phenyl- 1,2,3,4-tetrahydronaphthalen- 1-yl)-5-methoxyphenyl)-7-aza- spiro[3.5]nonan-2-yl)methyl)- 8-methyl-7-oxo-5,7-dihydro- 2H,6H-spiro[furo[2,3-f]iso- indole-3,4′-piperidin]-6-yl)- piperidine-2,6-dione |
| A220 | 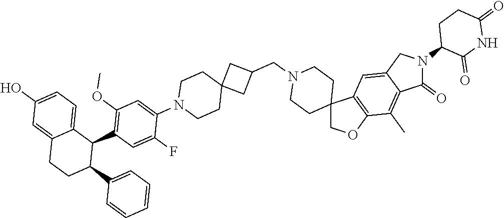 [SMILES: COc1cc(N2CCC3(CC2)CC(CN2CCC4(CC2)COc2c4cc4c(c2C)C(=O)N([C@H]2CCC(=O)NC2=O)C4)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen- 1-yl)-5-methoxyphenyl)-7-aza- spiro[3.5]nonan-2-yl)methyl)- 8-methyl-7-oxo-5,7-dihydro- 2H,6H-spiro[furo[2,3-f]iso- indole-3,4′-piperidin]-6-yl)- piperidine-2,6-dione |
| A221 | 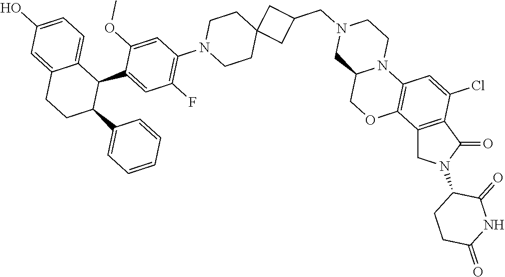 [SMILES: COc1cc(N2CCC3(CC2)CC(CN2CCN4c5cc(Cl)c6c(c5OC[C@H]4C2)CN([C@H]2CCC(=O)NC2=O)C6=O)C3)c(F)cc1[C@@H]1c2ccc(O)cc2CC[C@@H]1c1ccccc1] | (S)-3-((R)-12-chloro-7-((7-(2- fluoro-4-((1S,2S)-6-hydroxy-2- phenyl-1,2,3,4-tetrahydronaph- thalen-1-yl)-5-methoxyphenyl)- 7-azaspiro[3.5]nonan-2-yl)- methyl)-1-oxo-1,3,5,5a,6,7,8,9- octahydro-2H-pyrazino[1′,2′: 4,5][1,4]oxazino[2,3-e]isoindol- 2-yl)piperidine-2,6-dione |
| A222 | 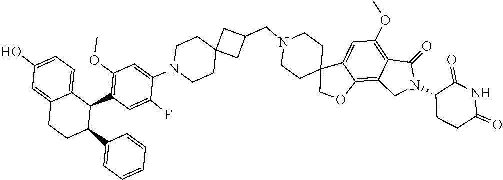 [SMILES: COc1cc(N2CCC3(CC2)CC(CN2CCC4(CC2)COc2c4cc(OC)c4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen- 1-yl)-5-methoxyphenyl)-7-aza- spiro[3.5]nonan-2-yl)mcthyl)- 5-mcthoxy-6-oxo-6,8-dihydro- 2H,7H-spiro[furo[2,3-e]iso- indole-3,4′-piperidin]-7-yl)- piperidine-2,6-dione |
| A223 | 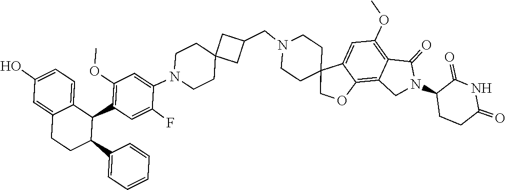 [SMILES: COc1cc(N2CCC3(CC2)CC(CN2CCC4(CC2)COc2c4cc(OC)c4c2CN([C@@H]2CCC(=O)NC2=O)C4=O)C3)c(F)cc1[C@@H]1c2ccc(O)cc2CC[C@@H]1c1ccccc1] | (R)-3-(1′-((7-(2-fluoro-4- ((1S,2S)-6-hydroxy-2-phenyl- 1,2,3,4-tetrahydronaphthalen- 1-yl)-5-methoxyphenyl)-7-aza- spiro[3.5]nonan-2-yl)methyl)- 5-methoxy-6-oxo-6,8-dihydro- 2H,7H-spiro[furo[2,3-e]iso- indole-3,4′-piperidin]-7-yl)- piperidine-2,6-dione |
| A224 | 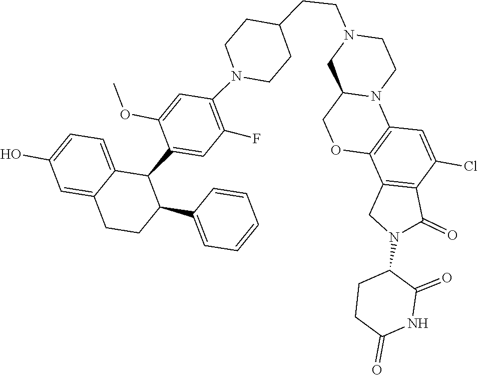 [SMILES: COc1cc(N2CCC(CCN3CCN4c5cc(Cl)c6c(c5OC[C@H]4C3)CN([C@H]3CCC(=O)NC3=O)C6=O)CC2)c(F)cc1[C@@H]1c2ccc(O)cc2CC[C@@H]1c1ccccc1] | (S)-3-((R)-12-chloro-7-(2-(1- (2-fluoro-4-((1S,2S)-6- hydroxy-2-phenyl-1,2,3,4- tetrahydronaphthalen-1-yl)-5- methoxyphenyl)piperidin-4- yl)ethyl)-1-oxo-1,3,5,5a,6,7,8, 9-octahydro-2H-pyrazino[1′,2′: 4,5][1,4]oxazino[2,3-e]isoindol- 2-yl)piperidine-2,6-dione |
| A225 | 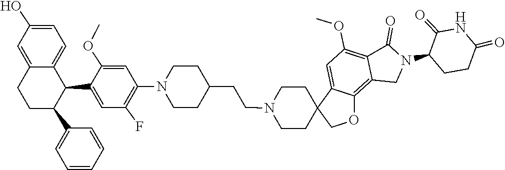 [SMILES: COc1cc(N2CCC(CCN3CCC4(CC3)COc3c4cc(OC)c4c3CN([C@@H]3CCC(=O)NC3=O)C4=O)CC2)c(F)cc1[C@@H]1c2ccc(O)cc2CC[C@@H]1c1ccccc1] | (R)-3-(1′-(2-(1-(2-fluoro-4- ((1S,2S)-6-hydroxy-2-phenyl- 1,2,3,4-tetrahydronaphthalen- 1-yl)-5-methoxyphenyl)piper- idin-4-yl)ethyl)-5-methoxy-6- oxo-6,8-dihydro-2H,7H-spiro [furo[2,3-e]isoindole-3,4′- piperidin]-7-yl)piperidine-2,6- dione |
| A226 | 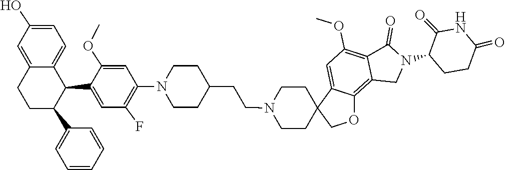 [SMILES: COc1cc(N2CCC(CCN3CCC4(CC3)COc3c4cc(OC)c4c3CN([C@H]3CCC(=O)NC3=O)C4=O)CC2)c(F)cc1[C@@H]1c2ccc(O)cc2CC[C@@H]1c1ccccc1] | (S)-3-(1′-(2-(1-(2-fluoro-4- ((1S,2S)-6-hydroxy-2-phenyl- 1,2,3,4-tetrahydronaphthalen- 1-yl)-5-methoxyphenyl)piper- idin-4-yl)ethyl)-5-methoxy-6- oxo-6,8-dihydro-2H,7H-spiro [furo[2,3-e]isoindole-3,4′- piperidin]-7-yl)piperidine-2,6- dione |
| A227 | 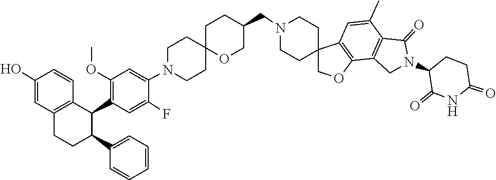 [SMILES: COc1cc(N2CCC3(CC[C@@H](CN4CCC5(CC4)COc4c5cc(C)c5c4CN([C@H]4CCC(=O)NC4=O)C5=O)CO3)CC2)c(F)cc1[C@@H]1c2ccc(O)cc2CC[C@@H]1c1ccccc1] | (S)-3-(1′-(((S)-9-(2-fluoro-4- ((1S,2S)-6-hydroxy-2-phenyl- 1,2,3,4-tetrahydronaphthalen- 1-yl)-5-methoxyphenyl)-1-oxa- 9-azaspiro[5.5]undccan-3-yl)- methyl)-5-methyl-6-oxo-6,8- dihydro-2H,7H-spiro[furo[2,3- e]isoindole-3,4′-piperidin]-7- yl)piperidine-2,6-dione |
| A228 | 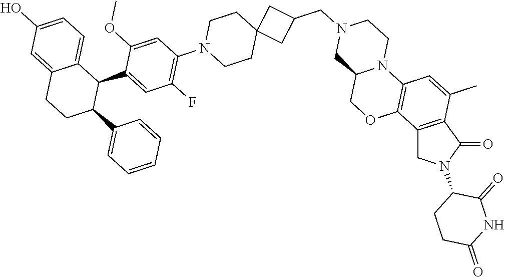 [SMILES: COc1cc(N2CCC3(CC2)CC(CN2CCN4c5cc(C)c6c(c5OC[C@H]4C2)CN([C@H]2CCC(=O)NC2=O)C6=O)C3)c(F)cc1[C@@H]1c2ccc(O)cc2CC[C@@H]1c1ccccc1] | (S)-3-((R)-7-((7-(2-fluoro-4- ((1S,2S)-6-hydroxy-2-phenyl- 1,2,3,4-tetrahydronaphthalen- 1-yl)-5-methoxyphenyl)-7-aza- spiro[3.5]nonan-2-yl)methyl)- 12-methyl-1-oxo-1,3,5,5a,6,7, 8,9-octahydro-2H-pyrazino [1′,2′:4,5][1,4]oxazino[2,3-e] isoindol-2-yl)piperidine-2,6- dione |
| A229 | 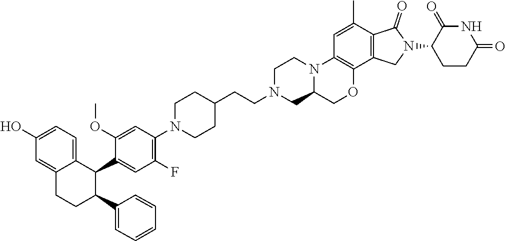 [SMILES: COc1cc(N2CCC(CCN3CCN4c5cc(C)c6c(c5OC[C@H]4C3)CN([C@H]3CCC(=O)NC3=O)C6=O)CC2)c(F)cc1[C@@H]1c2ccc(O)cc2CC[C@@H]1c1ccccc1] | (S)-3-((R)-7-(2-(1-(2-fluoro-4- ((1S,2S)-6-hydroxy-2-phenyl- 1,2,3,4-tetrahydronaphthalen- 1-yl)-5-methoxyphenyl)piper- idin-4-yl)ethyl)-12-methyl-1- oxo-1,3,5,5a,6,7,8,9-octahydro- 2H-pyrazino[1′,2′:4,5][1,4] oxazino[2,3-e]isoindol-2-yl)- piperidine-2,6-dione |
| A230 | 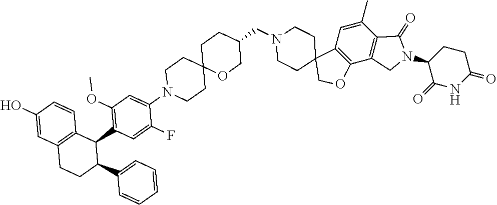 [SMILES: COc1cc(N2CCC3(CC[C@H](CN4CCC5(CC4)COc4c5cc(C)c5c4CN([C@H]4CCC(=O)NC4=O)C5=O)CO3)CC2)c(F)cc1[C@@H]1c2ccc(O)cc2CC[C@@H]1c1ccccc1] | (S)-3-(1′-(((R)-9-(2-fluoro-4- ((1S,2S)-6-hydroxy-2-phenyl- 1,2,3,4-tetrahydronaphthalen- 1-yl)-5-methoxyphenyl)-1-oxa- 9-azaspiro[5.5]undecan-3-yl)- methyl)-5-methyl-6-oxo-6,8- dihydro-2H,7H-spiro[furo[2,3- e]isoindole-3,4′-piperidin]-7- yl)piperidine-2,6-dione |
| A231 | 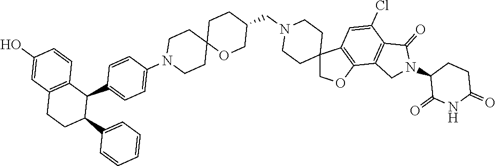 [SMILES: O=C1CC[C@H](N2Cc3c4c(cc(Cl)c3C2=O)C2(CCN(C[C@H]3CCC5(CCN(c6ccc([C@@H]7c8ccc(O)cc8CC[C@@H]7c7ccccc7)cc6)CC5)OC3)CC2)CO4)C(=O)N1] | (S)-3-(5-chloro-1′-(((R)-9-(4- ((1R,2S)-6-hydroxy-2-phenyl- 1,2,3,4-tetrahydronaphthalen- 1-yl)phenyl)-1-oxa-9-azaspiro [5.5]undecan-3-yl)methyl)-6- oxo-6,8-dihydro-2H,7H-spiro [furo[2,3-e]isoindole-3,4′- piperidin]-7-yl)piperidine-2,6- dione |
| A232 | 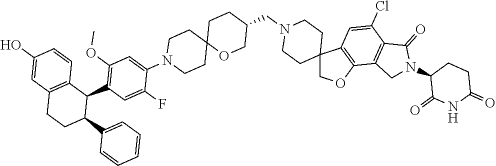 [SMILES: COc1cc(N2CCC3(CC[C@H](CN4CCC5(CC4)COc4c5cc(Cl)c5c4CN([C@H]4CCC(=O)NC4=O)C5=O)CO3)CC2)c(F)cc1[C@@H]1c2ccc(O)cc2CC[C@@H]1c1ccccc1] | (S)-3-(5-chloro-1′-(((R)-9-(2- fluoro-4-((1S,2S)-6-hydroxy-2- phenyl-1,2,3,4-tetrahydronaph- thalen-1-yl)-5-methoxyphenyl)- 1-oxa-9-azaspiro[5.5]undecan- 3-yl)methyl)-6-oxo-6,8- dihydro-2H,7H-spiro[furo[2,3- e]isoindole-3,4′-piperidin]-7- yl)piperidine-2,6-dione |
| A233 | 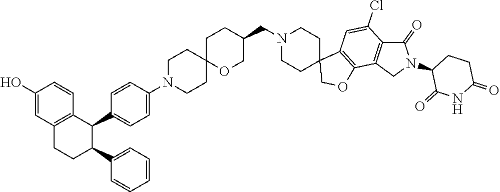 [SMILES: O=C1CC[C@H](N2Cc3c4c(cc(Cl)c3C2=O)C2(CCN(C[C@@H]3CCC5(CCN(c6ccc([C@@H]7c8ccc(O)cc8CC[C@@H]7c7ccccc7)cc6)CC5)OC3)CC2)CO4)C(=O)N1] | (S)-3-(5-chloro-1′-(((S)-9-(4- ((1R,2S)-6-hydroxy-2-phenyl- 1,2,3,4-tetrahydronaphthalen- 1-yl)phenyl)-1-oxa-9-azaspiro [5.5]undecan-3-yl)methyl)-6- oxo-6,8-dihydro-2H,7H-spiro [furo[2,3-e]isoindole-3,4′- piperidin]-7-yl)piperidine-2,6- dione |
| A234 | 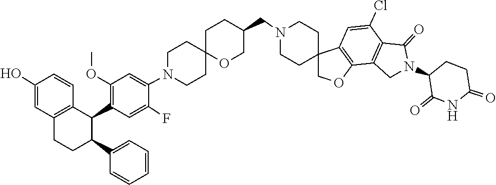 [SMILES: COc1cc(N2CCC3(CC[C@@H](CN4CCC5(CC4)COc4c5cc(Cl)c5c4CN([C@H]4CCC(=O)NC4=O)C5=O)CO3)CC2)c(F)cc1[C@@H]1c2ccc(O)cc2CC[C@@H]1c1ccccc1] | (S)-3-(5-chloro-1′-(((S)-9-(2- fluoro-4-((1S,2S)-6-hydroxy- 2-phenyl-1,2,3,4-tetrahydro- naphthalen-1-yl)-5-methoxy- phenyl)-1-oxa-9-azaspiro[5.5] undecan-3-yl)methyl)-6-oxo- 6,8-dihydro-2H,7H-spiro[furo [2,3-e]isoindole-3,4′-piperidin]- 7-yl)piperidine-2,6-dione |
| A235 | 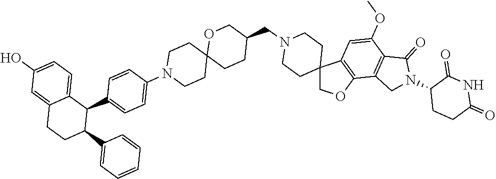 [SMILES: COc1cc2c(c3c1C(=O)N([C@H]1CCC(=O)NC1=O)C3)OCC21CCN(C[C@H]2CCC3(CCN(c4ccc([C@@H]5c6ccc(O)cc6CC[C@@H]5c5ccccc5)cc4)CC3)OC2)CC1] | (S)-3-(1′-(((R)-9-(4-((1R,2S)-6- hydroxy-2-phenyl-1,2,3,4-tetra- hydronaphthalen-1-yl)phenyl)- 1-oxa-9-azaspiro[5.5]undecan- 3-yl)methyl)-5-methoxy-6-oxo- 6,8-dihydro-2H,7H-spiro[furo [2,3-e]isoindole-3,4′-piperidin]- 7-yl)piperidine-2,6-dione |
| A236 | 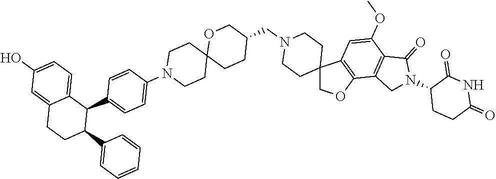 [SMILES: COc1cc2c(c3c1C(=O)N([C@H]1CCC(=O)NC1=O)C3)OCC21CCN(C[C@@H]2CCC3(CCN(c4ccc([C@@H]5c6ccc(O)cc6CC[C@@H]5c5ccccc5)cc4)CC3)OC2)CC1] | (S)-3-(1′-(((S)-9-(4-((1R,2S)-6- hydroxy-2-phenyl-1,2,3,4-tetra- hydronaphthalen-1-yl)phenyl)- 1-oxa-9-azaspiro[5.5]undecan- 3-yl)methyl)-5-methoxy-6-oxo- 6,8-dihydro-2H,7H-spiro[furo [2,3-e]isoindole-3,4′-piperidin]- 7-yl)piperidine-2,6-dione |

TABLE 2
*designates that it is racemic with respect to the two stereogenic centers at the warhead (tetrahydronaphthalene); the relative
configuration of the two stereogenic center is cis.

| Compound No. | Chemical Structure | Chemical Name |
|---|---|---|
| B134 | 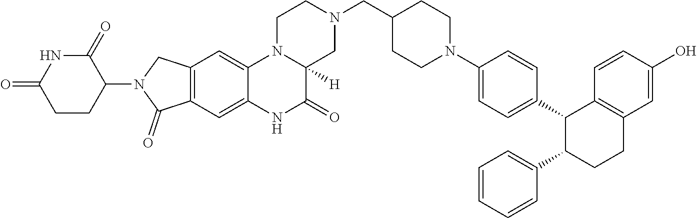 [SMILES: O=C1CCC(N2Cc3cc4c(cc3C2=O)NC(=O)[C@@H]2CN(CC3CCN(c5ccc([C@@H]6c7ccc(O)cc7CC[C@@H]6c6ccccc6)cc5)CC3)CCN42)C(=O)N1] | (4aS)-9-(2,6-dioxopiperidin-3-yl)-3-((1-(4- ((1R,2S)-6-hydroxy-2-phenyl-1,2,3,4- tetrahydronaphthalen-1- yl)phenyl)piperidin-4-yl)methyl)- 2,3,4,4a,9,10-hexahydropyrazino[1,2- a]pyrrolo[3,4-g]quinoxaline-5,8(1H,6H)- dione |
| B135 | 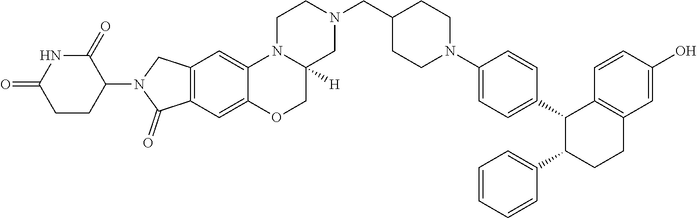 [SMILES: O=C1CCC(N2Cc3cc4c(cc3C2=O)OC[C@@H]2CN(CC3CCN(c5ccc([C@@H]6c7ccc(O)cc7CC[C@@H]6c6ccccc6)cc5)CC3)CCN42)C(=O)N1] | 3-((S)-3-((1-(4-((1R,2S)-6-hydroxy-2- phenyl-1,2,3,4-tetrahydronaphthalen-1- yl)phenyl)piperidin-4-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B135X | 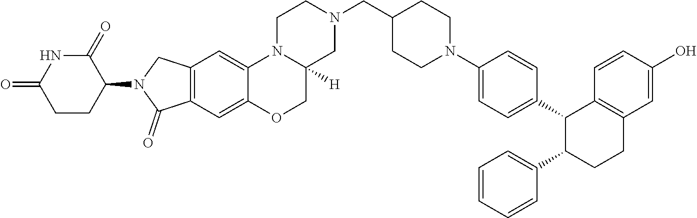 [SMILES: O=C1CC[C@H](N2Cc3cc4c(cc3C2=O)OC[C@@H]2CN(CC3CCN(c5ccc([C@@H]6c7ccc(O)cc7CC[C@@H]6c6ccccc6)cc5)CC3)CCN42)C(=O)N1] | (S)-3-((S)-3-((1-(4-((1R,2S)-6-hydroxy-2- phenyl-1,2,3,4-tetrahydronaphthalen-1- yl)phenyl)piperidin-4-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B137 | 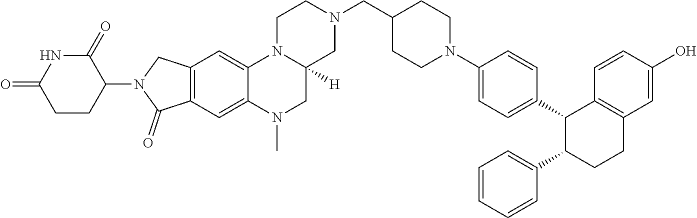 [SMILES: CN1C[C@@H]2CN(CC3CCN(c4ccc([C@@H]5c6ccc(O)cc6CC[C@@H]5c5ccccc5)cc4)CC3)CCN2c2cc3c(cc21)C(=O)N(C1CCC(=O)NC1=O)C3] | 3-((S)-3-((1-(4-((1R,2S)-6-hydroxy-2- phenyl-1,2,3,4-tetrahydronaphthalen-1- yl)phenyl)piperidin-4-yl)methyl)-6- methyl-8-oxo-2,3,4,4a,5,6,8,10- octahydropyrazino[1,2-a]pyrrolo[3,4- g]quinoxalin-9(1H)-yl)piperidine-2,6- dione |
| B140 | 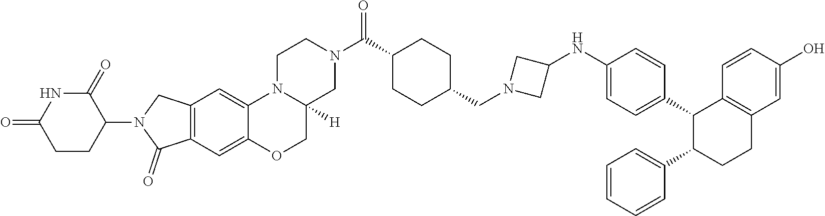 [SMILES: O=C1CCC(N2Cc3cc4c(cc3C2=O)OC[C@@H]2CN(C(=O)[C@H]3CC[C@@H](CN5CC(Nc6ccc([C@@H]7c8ccc(O)cc8CC[C@@H]7c7ccccc7)cc6)C5)CC3)CCN42)C(=O)N1] | 3-((S)-3-((1R,4S)-4-((3-((4-((1R,2S)-6- hydroxy-2-phenyl-1,2,3,4- tetrahydronaphthalen-1- yl)phenyl)amino)azetidin-1- yl)methyl)cyclohexane-1-carbonyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B141 | 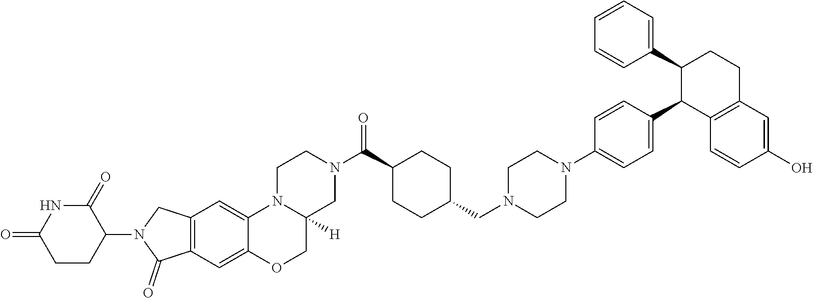 [SMILES: O=C1CCC(N2Cc3cc4c(cc3C2=O)OC[C@@H]2CN(C(=O)[C@H]3CC[C@H](CN5CCN(c6ccc([C@@H]7c8ccc(O)cc8CC[C@@H]7c7ccccc7)cc6)CC5)CC3)CCN42)C(=O)N1] | 3-((S)-3-((1R,4S)-4-((4-(4-((1R,2S)-6- hydroxy-2-phenyl-1,2,3,4- tetrahydronaphthalen-1- yl)phenyl)piperazin-1- yl)methyl)cyclohexane-1-carbonyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B142 | 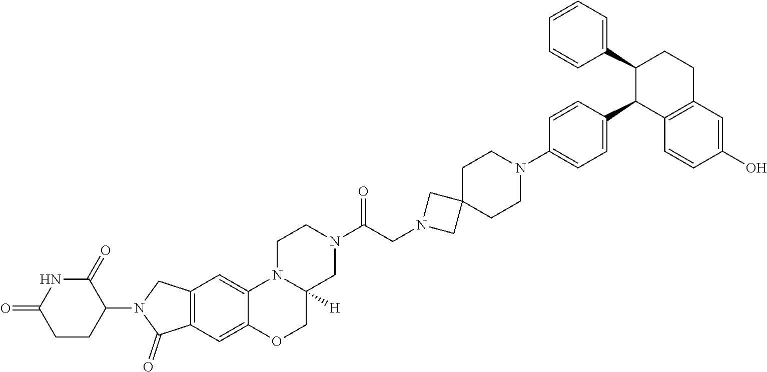 [SMILES: O=C1CCC(N2Cc3cc4c(cc3C2=O)OC[C@@H]2CN(C(=O)CN3CC5(CCN(c6ccc([C@@H]7c8ccc(O)cc8CC[C@@H]7c7ccccc7)cc6)CC5)C3)CCN42)C(=O)N1] | 3-((S)-3-(2-(7-(4-((1R,2S)-6-hydroxy-2- phenyl-1,2,3,4-tctrahydronaphthalen-1- yl)phenyl)-2,7-diazaspiro[3.5]nonan-2- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B143 | 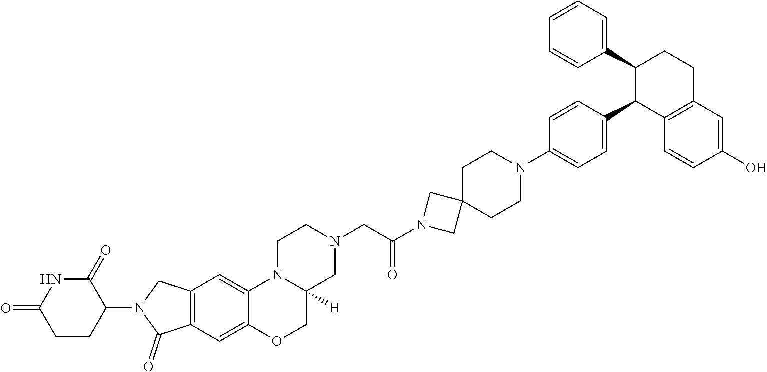 [SMILES: O=C1CCC(N2Cc3cc4c(cc3C2=O)OC[C@@H]2CN(CC(=O)N3CC5(CCN(c6ccc([C@@H]7c8ccc(O)cc8CC[C@@H]7c7ccccc7)cc6)CC5)C3)CCN42)C(=O)N1] | 3-((S)-3-(2-(7-(4-((1R,2S)-6-hydroxy-2- phenyl-1,2,3,4-tetrahydronaphthalen-1- yl)phenyl)-2,7-diazaspiro[3.5]nonan-2-yl)- 2-oxoethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B144 | 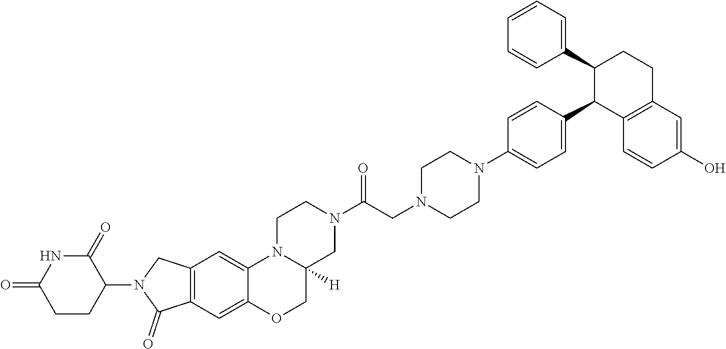 [SMILES: O=C1CCC(N2Cc3cc4c(cc3C2=O)OC[C@@H]2CN(C(=O)CN3CCN(c5ccc([C@@H]6c7ccc(O)cc7CC[C@@H]6c6ccccc6)cc5)CC3)CCN42)C(=O)N1] | 3-((S)-3-(2-(4-(4-((1R,2S)-6-hydroxy-2- phenyl-1,2,3,4-tetrahydronaphthalen-1- yl)phenyl)piperazin-1-yl)acetyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B145 | 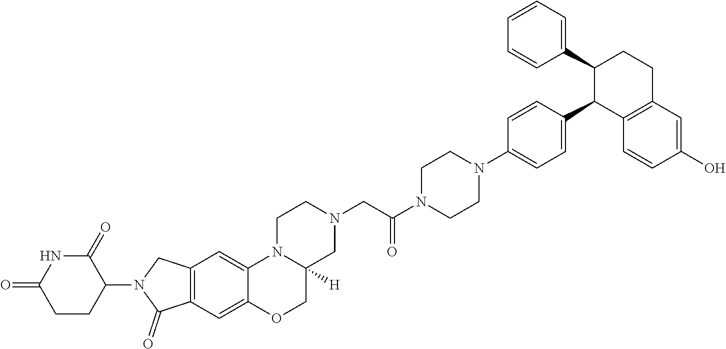 [SMILES: O=C1CCC(N2Cc3cc4c(cc3C2=O)OC[C@@H]2CN(CC(=O)N3CCN(c5ccc([C@@H]6c7ccc(O)cc7CC[C@@H]6c6ccccc6)cc5)CC3)CCN42)C(=O)N1] | 3-((S)-3-(2-(4-(4-((1R,2S)-6-hydroxy-2- phenyl-1,2,3,4-tetrahydronaphthalen-1- yl)phenyl)piperazin-1-yl)-2-oxoethyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| B146 | 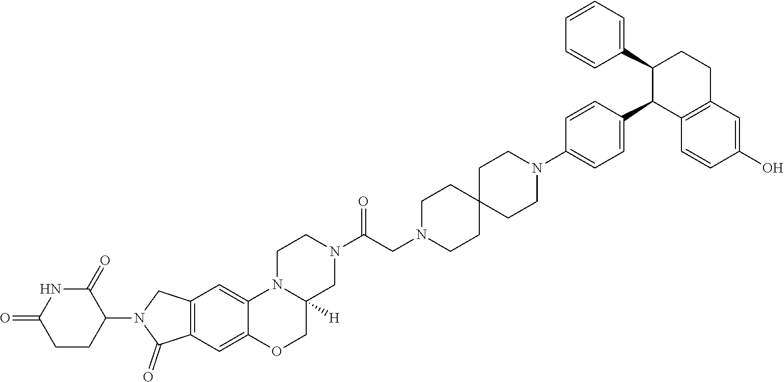 [SMILES: O=C1CCC(N2Cc3cc4c(cc3C2=O)OC[C@@H]2CN(C(=O)CN3CCC5(CC3)CCN(c3ccc([C@@H]6c7ccc(O)cc7CC[C@@H]6c6ccccc6)cc3)CC5)CCN42)C(=O)N1] | 3-((S)-3-(2-(9-(4-((1R,2S)-6-hydroxy-2- phenyl-1,2,3,4-tetrahydronaphthalen-1- yl)phenyl)-3,9-diazaspiro[5.5]undecan-3- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B147 | 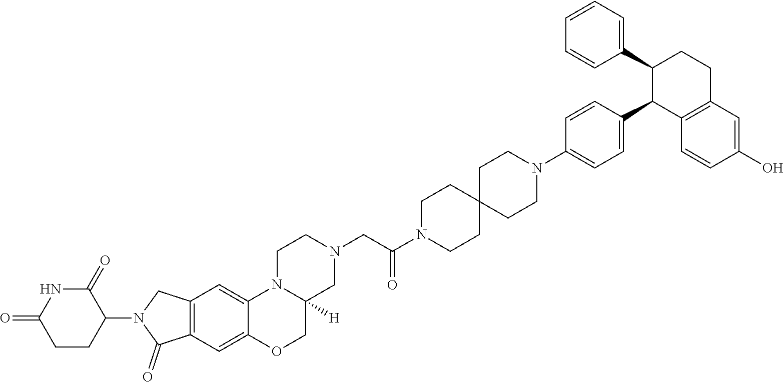 [SMILES: O=C1CCC(N2Cc3cc4c(cc3C2=O)OC[C@@H]2CN(CC(=O)N3CCC5(CC3)CCN(c3ccc([C@@H]6c7ccc(O)cc7CC[C@@H]6c6ccccc6)cc3)CC5)CCN42)C(=O)N1] | 3-((S)-3-(2-(9-(4-((1R,2S)-6-hydroxy-2- phenyl-1,2,3,4-tetrahydronaphthalen-1- yl)phenyl)-3,9-diazaspiro[5.5]undecan-3- yl)-2-oxoethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B148* | 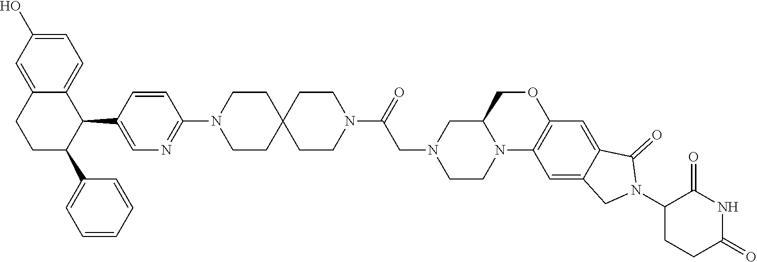 [SMILES: O=C1CCC(N2Cc3cc4c(cc3C2=O)OC[C@@H]2CN(CC(=O)N3CCC5(CC3)CCN(c3ccc([C@@H]6c7ccc(O)cc7CC[C@@H]6c6ccccc6)cn3)CC5)CCN42)C(=O)N1] | 3-((S)-3-(2-(9-(5-((1S,2S)-6-hydroxy-2- phenyl-1,2,3,4-tetrahydronaphthalen-1- yl)pyridin-2-yl)-3,9- diazaspiro[5.5]undecan-3-yl)-2-oxoethyl)- 8-oxo-1,2,3,4,4a,5,8,10-octahydro-9H- pyrazino[1′,2′:4,5][1,4]oxazino[2,3- f]isoindol-9-yl)piperidine-2,6-dione |
| B149* | 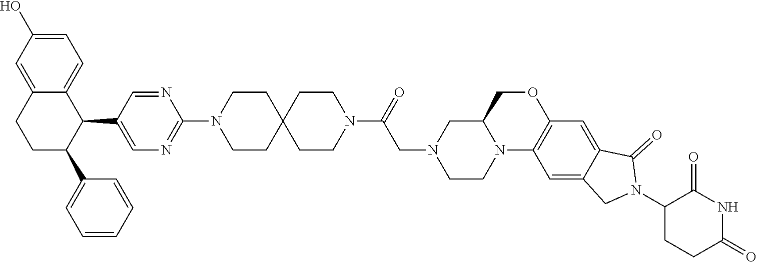 [SMILES: O=C1CCC(N2Cc3cc4c(cc3C2=O)OC[C@@H]2CN(CC(=O)N3CCC5(CC3)CCN(c3ncc([C@@H]6c7ccc(O)cc7CC[C@@H]6c6ccccc6)cn3)CC5)CCN42)C(=O)N1] | 3-((S)-3-(2-(9-(5-((1S,2S)-6-hydroxy-2- phenyl-1,2,3,4-tetrahydronaphthalen-1- yl)pyrimidin-2-yl)-3,9- diazaspiro[5.5]undecan-3-yl)-2-oxoethyl)- 8-oxo-1,2,3,4,4a,5,8,10-octahydro-9H- pyrazino[1′,2′:4,5][1,4]oxazino[2,3- f]isoindol-9-yl)piperidine-2,6-dione |
| B150 | 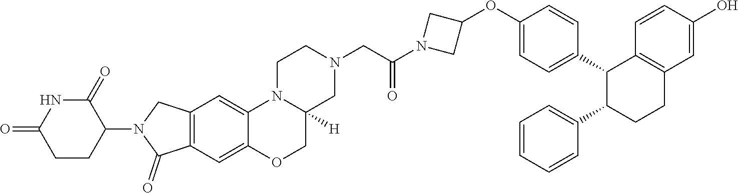 [SMILES: O=C1CCC(N2Cc3cc4c(cc3C2=O)OC[C@@H]2CN(CC(=O)N3CC(Oc5ccc([C@@H]6c7ccc(O)cc7CC[C@@H]6c6ccccc6)cc5)C3)CCN42)C(=O)N1] | 3-((S)-3-(2-(3-(4-((1R,2S)-6-hydroxy-2- phenyl-1,2,3,4-tetrahydronaphthalen-1- yl)phenoxy)azetidin-1-yl)-2-oxoethyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| B151 | 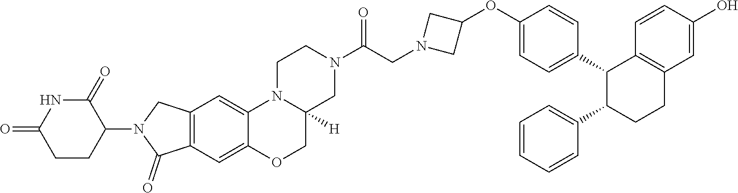 [SMILES: O=C1CCC(N2Cc3cc4c(cc3C2=O)OC[C@@H]2CN(C(=O)CN3CC(Oc5ccc([C@@H]6c7ccc(O)cc7CC[C@@H]6c6ccccc6)cc5)C3)CCN42)C(=O)N1] | 3-((S)-3-(2-(3-(4-((1R,2S)-6-hydroxy-2- phenyl-1,2,3,4-tetrahydronaphthalen-1- yl)phenoxy)azetidin-1-yl)acetyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B152 | 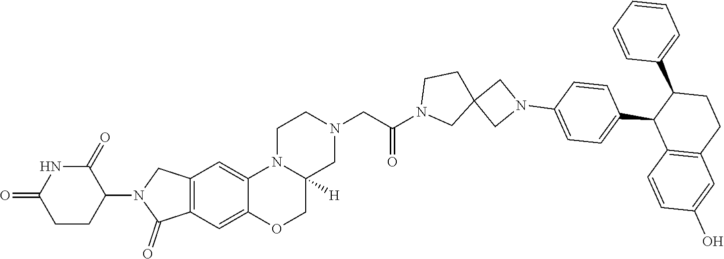 [SMILES: O=C1CCC(N2Cc3cc4c(cc3C2=O)OC[C@@H]2CN(CC(=O)N3CCC5(C3)CN(c3ccc([C@@H]6c7ccc(O)cc7CC[C@@H]6c6ccccc6)cc3)C5)CCN42)C(=O)N1] | 3-((S)-3-(2-(2-(4-((1R,2S)-6-hydroxy-2- phenyl-1,2,3,4-tetrahydronaphthalen-1- yl)phenyl)-2,6-diazaspiro[3.4]octan-6-yl)- 2-oxoethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B153 | 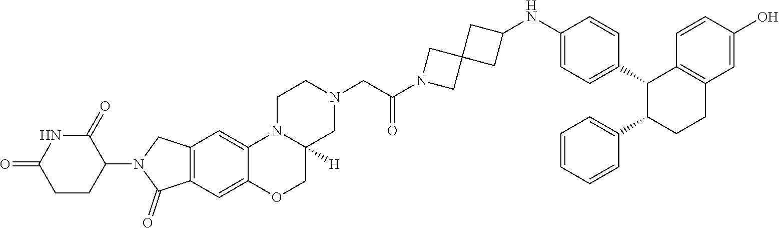 [SMILES: O=C1CCC(N2Cc3cc4c(cc3C2=O)OC[C@@H]2CN(CC(=O)N3CC5(CC(Nc6ccc([C@@H]7c8ccc(O)cc8CC[C@@H]7c7ccccc7)cc6)C5)C3)CCN42)C(=O)N1] | 3-((S)-3-(2-(6-((4-((1R,2S)-6-hydroxy-2- phenyl-1,2,3,4-tetrahydronaphthalen-1- yl)phenyl)amino)-2-azaspiro[3.3]heptan-2- yl)-2-oxoethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B154 | 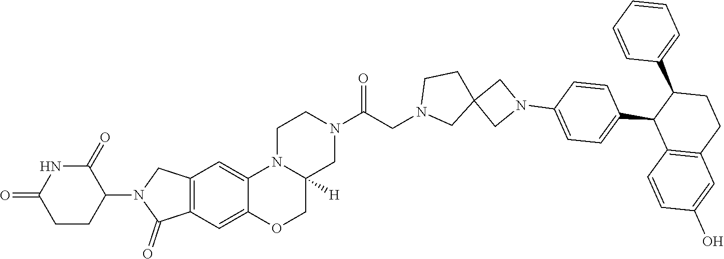 [SMILES: O=C1CCC(N2Cc3cc4c(cc3C2=O)OC[C@@H]2CN(C(=O)CN3CCC5(C3)CN(c3ccc([C@@H]6c7ccc(O)cc7CC[C@@H]6c6ccccc6)cc3)C5)CCN42)C(=O)N1] | 3-((S)-3-(2-(2-(4-((1R,2S)-6-hydroxy-2- phenyl-1,2,3,4-tetrahydronaphthalen-1- yl)phenyl)-2,6-diazaspiro[3.4]octan-6- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B155 | 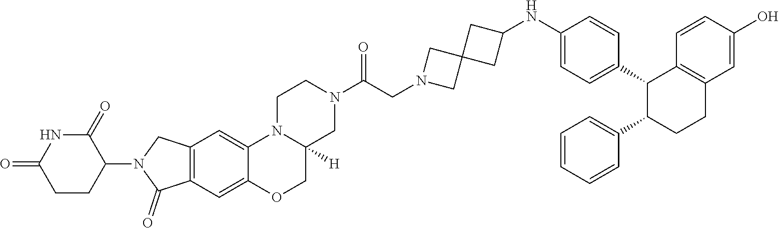 [SMILES: O=C1CCC(N2Cc3cc4c(cc3C2=O)OC[C@@H]2CN(C(=O)CN3CC5(CC(Nc6ccc([C@@H]7c8ccc(O)cc8CC[C@@H]7c7ccccc7)cc6)C5)C3)CCN42)C(=O)N1] | 3-((S)-3-(2-(6-((4-((1R,2S)-6-hydroxy-2- phenyl-1,2,3,4-tetrahydronaphthalen-1- yl)phenyl)amino)-2-azaspiro[3.3]heptan-2- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B156 | 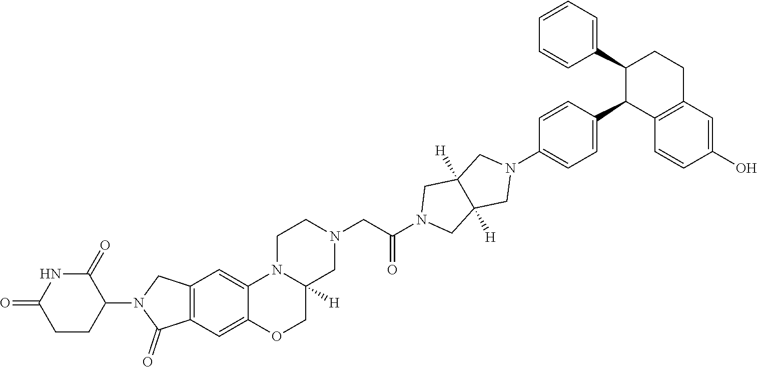 [SMILES: O=C1CCC(N2Cc3cc4c(cc3C2=O)OC[C@@H]2CN(CC(=O)N3C[C@@H]5CN(c6ccc([C@@H]7c8ccc(O)cc8CC[C@@H]7c7ccccc7)cc6)C[C@@H]5C3)CCN42)C(=O)N1] | 3-((S)-3-(2-((3aR,6aS)-5-(4-((1R,2S)-6- hydroxy-2-phenyl-1,2,3,4- tetrahydronaphthalen-1- yl)phenyl)hexahydropyrrolo[3,4-c]pyrrol- 2(1H)-yl)-2-oxoethyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B157 | 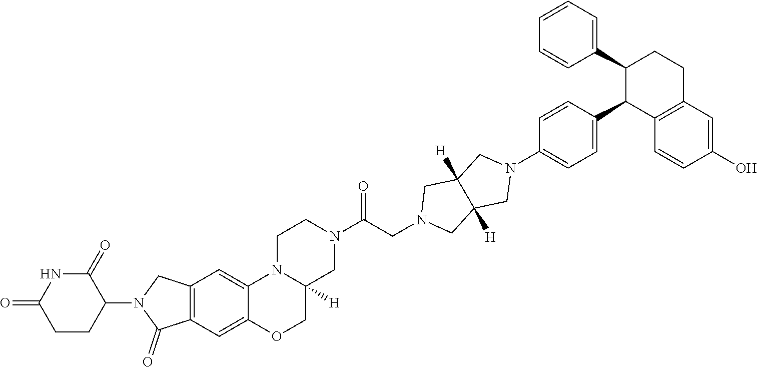 [SMILES: O=C1CCC(N2Cc3cc4c(cc3C2=O)OC[C@@H]2CN(C(=O)CN3C[C@@H]5CN(c6ccc([C@@H]7c8ccc(O)cc8CC[C@@H]7c7ccccc7)cc6)C[C@@H]5C3)CCN42)C(=O)N1] | 3-((S)-3-(2-((3aR,6aS)-5-(4-((1R,2S)-6- hydroxy-2-phenyl-1,2,3,4- tetrahydronaphthalen-1- yl)phenyl)hexahydropyrrolo[3,4-c]pyrrol- 2(1H)-yl)acetyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B158* | 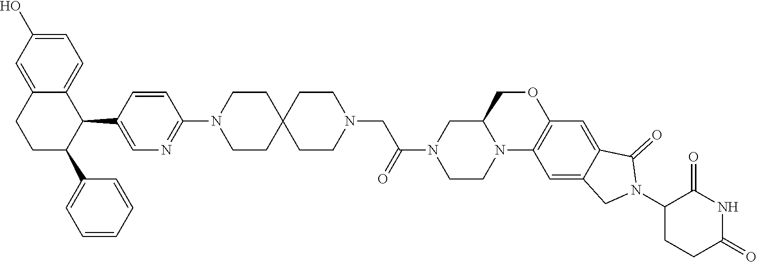 [SMILES: O=C1CCC(N2Cc3cc4c(cc3C2=O)OC[C@@H]2CN(C(=O)CN3CCC5(CC3)CCN(c3ccc([C@@H]6c7ccc(O)cc7CC[C@@H]6c6ccccc6)cn3)CC5)CCN42)C(=O)N1] | 3-((S)-3-(2-(9-(5-((1S,2S)-6-hydroxy-2- phenyl-1,2,3,4-tetrahydronaphthalen-1- yl)pyridin-2-yl)-3,9- diazaspiro[5.5]undecan-3-yl)acetyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| B159* | 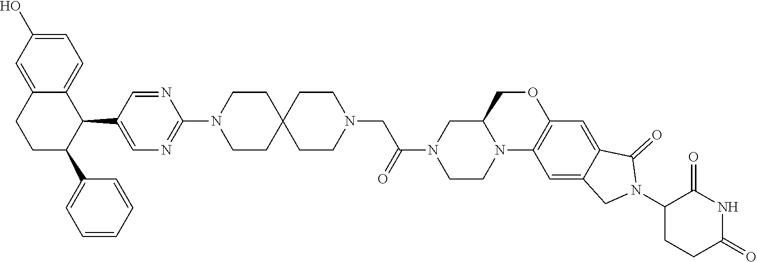 [SMILES: O=C1CCC(N2Cc3cc4c(cc3C2=O)OC[C@@H]2CN(C(=O)CN3CCC5(CC3)CCN(c3ncc([C@@H]6c7ccc(O)cc7CC[C@@H]6c6ccccc6)cn3)CC5)CCN42)C(=O)N1] | 3-((S)-3-(2-(9-(5-((1S,2S)-6-hydroxy-2- phenyl-1,2,3,4-tetrahydronaphthalen-1- yl)pyrimidin-2-yl)-3,9- diazaspiro[5.5]undecan-3-yl)acetyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| B160 | 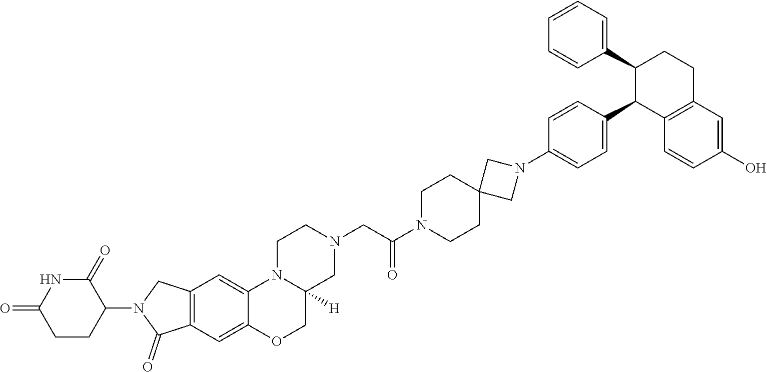 [SMILES: O=C1CCC(N2Cc3cc4c(cc3C2=O)OC[C@@H]2CN(CC(=O)N3CCC5(CC3)CN(c3ccc([C@@H]6c7ccc(O)cc7CC[C@@H]6c6ccccc6)cc3)C5)CCN42)C(=O)N1] | 3-((S)-3-(2-(2-(4-((1R,2S)-6-hydroxy-2- phenyl-1,2,3,4-tetrahydronaphthalen-1- yl)phenyl)-2,7-diazaspiro[3.5]nonan-7-yl)- 2-oxoethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B161 | 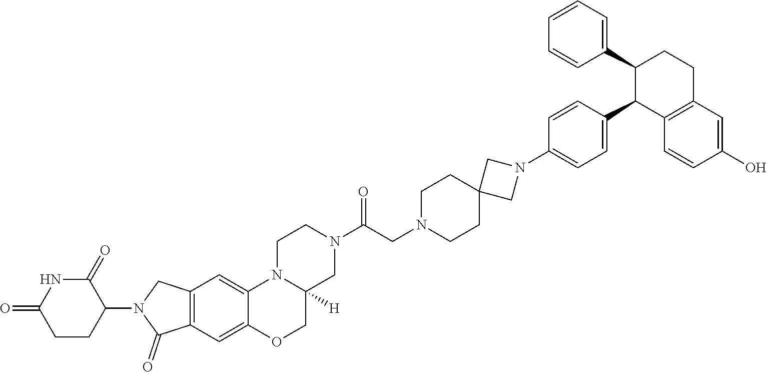 [SMILES: O=C1CCC(N2Cc3cc4c(cc3C2=O)OC[C@@H]2CN(C(=O)CN3CCC5(CC3)CN(c3ccc([C@@H]6c7ccc(O)cc7CC[C@@H]6c6ccccc6)cc3)C5)CCN42)C(=O)N1] | 3-((S)-3-(2-(2-(4-((1R,2S)-6-hydroxy-2- phenyl-1,2,3,4-tetrahydronaphthalen-1- yl)phenyl)-2,7-diazaspiro[3.5]nonan-7- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B162 | 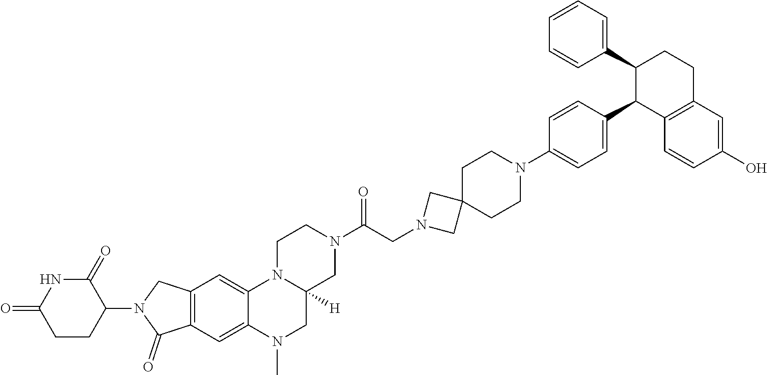 [SMILES: CN1C[C@@H]2CN(C(=O)CN3CC4(CCN(c5ccc([C@@H]6c7ccc(O)cc7CC[C@@H]6c6ccccc6)cc5)CC4)C3)CCN2c2cc3c(cc21)C(=O)N(C1CCC(=O)NC1=O)C3] | 3-((R)-3-(2-(7-(4-((1R,2S)-6-hydroxy-2- phenyl-1,2,3,4-tetrahydronaphthalen-1- yl)phenyl)-2,7-diazaspiro[3.5]nonan-2- yl)acetyl)-6-methyl-8-oxo- 2,3,4,4a,5,6,8,10-octahydropyrazino[1,2- a]pyrrolo[3,4-g]quinoxalin-9(1H)- yl)piperidine-2,6-dione |
| B164 | 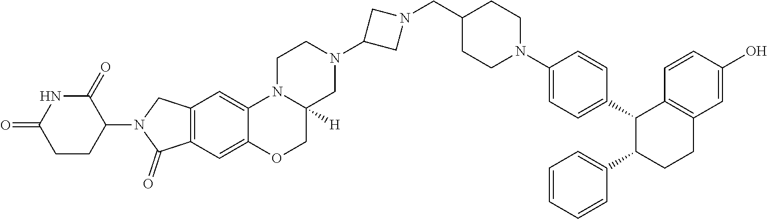 [SMILES: O=C1CCC(N2Cc3cc4c(cc3C2=O)OC[C@@H]2CN(C3CN(CC5CCN(c6ccc([C@@H]7c8ccc(O)cc8CC[C@@H]7c7ccccc7)cc6)CC5)C3)CCN42)C(=O)N1] | 3-((S)-3-(1-((1-(4-((1R,2S)-6-hydroxy-2- phenyl-1,2,3,4-tetrahydronaphthalen-1- yl)phenyl)piperidin-4-yl)methyl)azetidin- 3-yl)-8-oxo-1,2,3,4,4a,5,8,10-octahydro- 9H-pyrazino[1′,2′:4,5][1,4]oxazino[2,3- f]isoindol-9-yl)piperidine-2,6-dione |
| B165 | 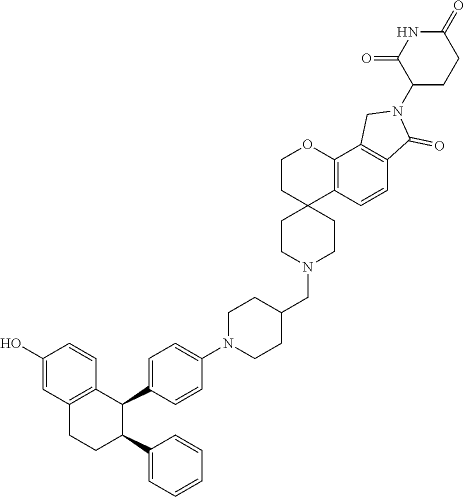 [SMILES: O=C1CCC(N2Cc3c(ccc4c3OCCC43CCN(CC4CCN(c5ccc([C@@H]6c7ccc(O)cc7CC[C@@H]6c6ccccc6)cc5)CC4)CC3)C2=O)C(=O)N1] | 3-(1-((1-(4-((1R,2S)-6-hydroxy-2-phenyl- 1,2,3,4-tetrahydronaphthalen-1- yl)phenyl)piperidin-4-yl)methyl)-7′-oxo- 2′,3′,7′,9′-tetrahydro-8′H-spiro[piperidine- 4,4′-pyrano[2,3-e]isoindol]-8′- yl)piperidine-2,6-dione |
| B167 | 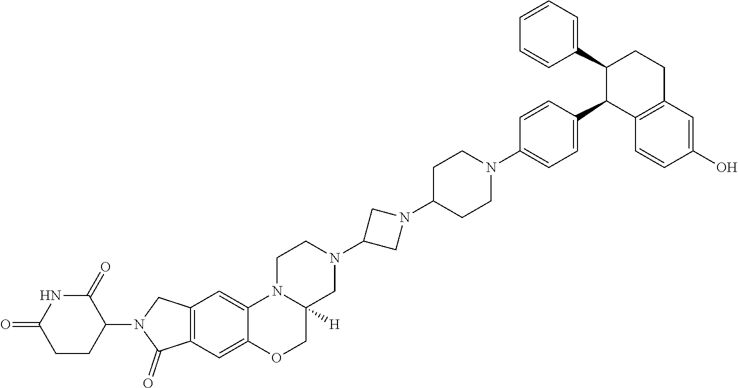 [SMILES: O=C1CCC(N2Cc3cc4c(cc3C2=O)OC[C@@H]2CN(C3CN(C5CCN(c6ccc([C@@H]7c8ccc(O)cc8CC[C@@H]7c7ccccc7)cc6)CC5)C3)CCN42)C(=O)N1] | 3-((S)-3-(1-(1-(4-((1R,2S)-6-hydroxy-2- phenyl-1,2,3,4-tetrahydronaphthalen-1- yl)phenyl)piperidin-4-yl)azetidin-3-yl)-8- oxo-1,2,3,4,4a,5,8,10-octahydro-9H- pyrazino[1′,2′:4,5][1,4]oxazino[2,3- f]isoindol-9-yl)piperidine-2,6-dione |
| B168 | 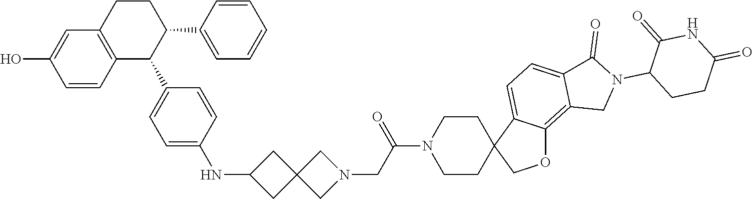 [SMILES: O=C1CCC(N2Cc3c(ccc4c3OCC43CCN(C(=O)CN4CC5(CC(Nc6ccc([C@@H]7c8ccc(O)cc8CC[C@@H]7c7ccccc7)cc6)C5)C4)CC3)C2=O)C(=O)N1] | 3-(1′-(2-(6-((4-((1R,2S)-6-hydroxy-2- phenyl-1,2,3,4-tetrahydronaphthalen-1- yl)phenyl)amino)-2-azaspiro[3.3]heptan-2- yl)acetyl)-6-oxo-6,8-dihydro-2H,7H- spiro[furo[2,3-e]isoindole-3,4′-piperidin]- 7-yl)piperidine-2,6-dione |
| B169 | 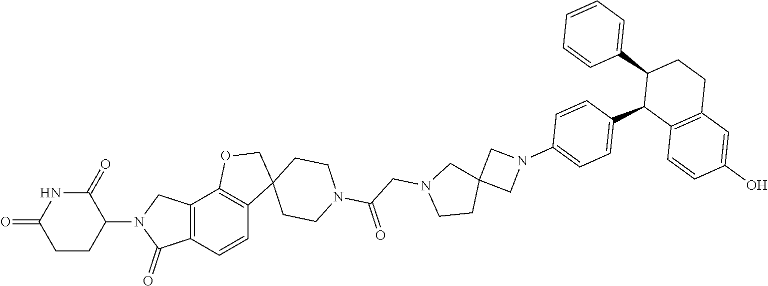 [SMILES: O=C1CCC(N2Cc3c(ccc4c3OCC43CCN(C(=O)CN4CCC5(C4)CN(c4ccc([C@@H]6c7ccc(O)cc7CC[C@@H]6c6ccccc6)cc4)C5)CC3)C2=O)C(=O)N1] | 3-(1′-(2-(2-(4-((1R,2S)-6-hydroxy-2- phenyl-1,2,3,4-tetrahydronaphthalen-1- yl)phenyl)-2,6-diazaspiro[3.4]octan-6- yl)acetyl)-6-oxo-6,8-dihydro-2H,7H- spiro[furo[2,3-e]isoindole-3,4′-piperidin]- 7-yl)piperidine-2,6-dione |
| B170 | 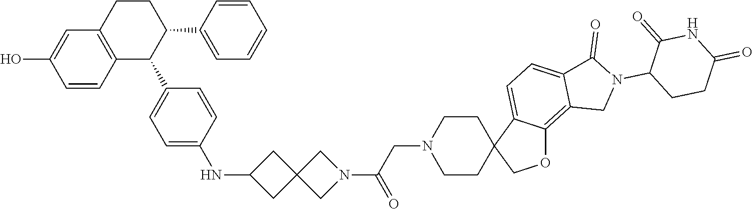 [SMILES: O=C1CCC(N2Cc3c(ccc4c3OCC43CCN(CC(=O)N4CC5(CC(Nc6ccc([C@@H]7c8ccc(O)cc8CC[C@@H]7c7ccccc7)cc6)C5)C4)CC3)C2=O)C(=O)N1] | 3-(1′-(2-(6-((4-((1R,2S)-6-hydroxy-2- phenyl-1,2,3,4-tetrahydronaphthalen-1- yl)phenyl)amino)-2-azaspiro[3.3]heptan-2- yl)-2-oxoethyl)-6-oxo-6,8-dihydro-2H,7H- spiro[furo[2,3-e]isoindole-3,4′-piperidin]- 7-yl)piperidine-2,6-dione |
| B171 | 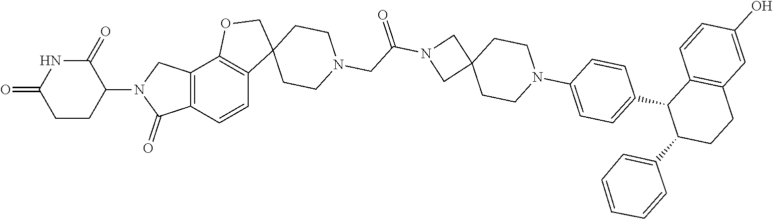 [SMILES: O=C1CCC(N2Cc3c(ccc4c3OCC43CCN(CC(=O)N4CC5(CCN(c6ccc([C@@H]7c8ccc(O)cc8CC[C@@H]7c7ccccc7)cc6)CC5)C4)CC3)C2=O)C(=O)N1] | 3-(1′-(2-(7-(4-((1R,2S)-6-hydroxy-2- phenyl-1,2,3,4-tetrahydronaphthalen-1- yl)phenyl)-2,7-diazaspiro[3.5]nonan-2-yl)- 2-oxoethyl)-6-oxo-6,8-dihydro-2H,7H- spiro[furo[2,3-e]isoindole-3,4′-piperidin]- 7-yl)piperidine-2,6-dione |
| B172 | 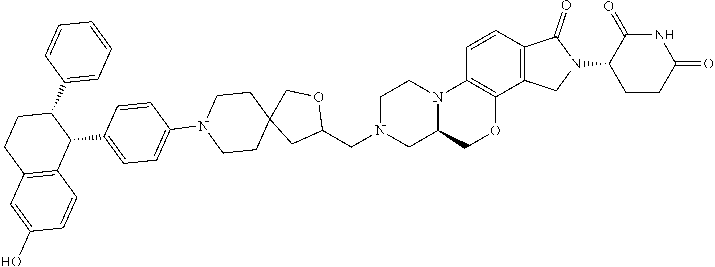 [SMILES: O=C1CC[C@H](N2Cc3c(ccc4c3OC[C@H]3CN(CC5CC6(CCN(c7ccc([C@@H]8c9ccc(O)cc9CC[C@@H]8c8ccccc8)cc7)CC6)CO5)CCN43)C2=O)C(=O)N1] | (3S)-3-((5aR)-7-((8-(4-((1R,2S)-6- hydroxy-2-phenyl-1,2,3,4- tetrahydronaphthalen-1-yl)phenyl)-2-oxa- 8-azaspiro[4.5]decan-3-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B173 | 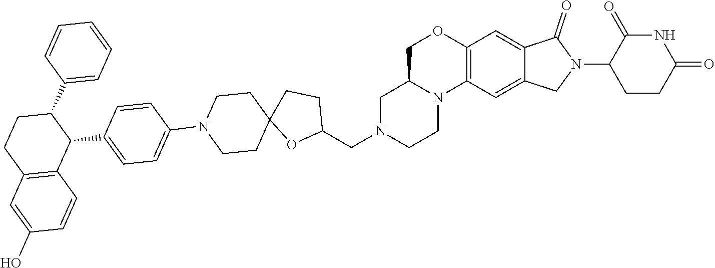 [SMILES: O=C1CCC(N2Cc3cc4c(cc3C2=O)OC[C@@H]2CN(CC3CCC5(CCN(c6ccc([C@@H]7c8ccc(O)cc8CC[C@@H]7c7ccccc7)cc6)CC5)O3)CCN42)C(=O)N1] | 3-((4aS)-3-((8-(4-((1R,2S)-6-hydroxy-2- phenyl-1,2,3,4-tetrahydronaphthalen-1- yl)phenyl)-2-oxa-8-azaspiro[4.5]decan-3- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B174 | 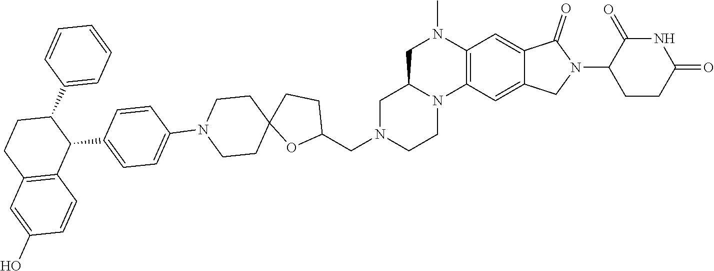 [SMILES: CN1C[C@@H]2CN(CC3CCC4(CCN(c5ccc([C@@H]6c7ccc(O)cc7CC[C@@H]6c6ccccc6)cc5)CC4)O3)CCN2c2cc3c(cc21)C(=O)N(C1CCC(=O)NC1=O)C3] | 3-((4aS)-3-((8-(4-((1R,2S)-6-hydroxy-2- phenyl-1,2,3,4-tetrahydronaphthalen-1- yl)phenyl)-2-oxa-8-azaspiro[4.5]decan-3- yl)methyl)-6-methyl-8-oxo- 2,3,4,4a,5,6,8,10-octahydropyrazino[1,2- a]pyrrolo[3,4-g]quinoxalin-9(1H)- yl)piperidine-2,6-dione |
| B175 | 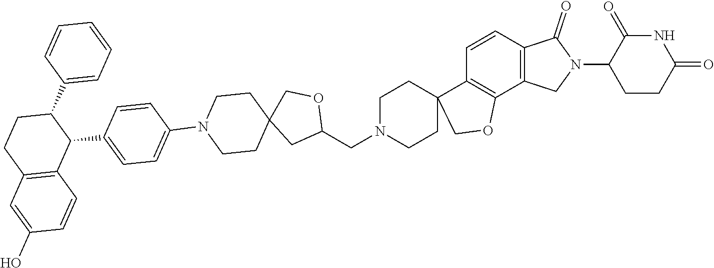 [SMILES: O=C1CCC(N2Cc3c(ccc4c3OCC43CCN(CC4CC5(CCN(c6ccc([C@@H]7c8ccc(O)cc8CC[C@@H]7c7ccccc7)cc6)CC5)CO4)CC3)C2=O)C(=O)N1] | 3-(1′-((8-(4-((1R,2S)-6-hydroxy-2-phenyl- 1,2,3,4-tetrahydronaphthalen-1-yl)phenyl)- 2-oxa-8-azaspiro[4.5]decan-3-yl)methyl)- 6-oxo-6,8-dihydro-2H,7H-spiro[furo[2,3- e]isoindole-3,4′-piperidin]-7-yl)piperidine- 2,6-dione |
| B176 | 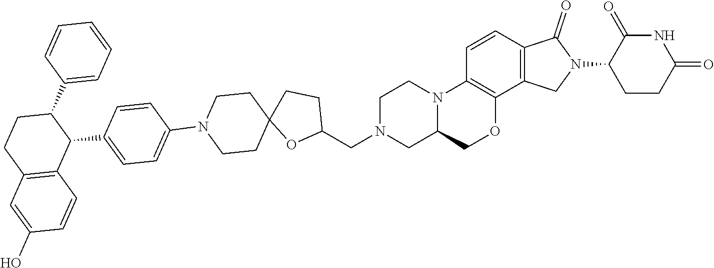 [SMILES: O=C1CC[C@H](N2Cc3c(ccc4c3OC[C@H]3CN(CC5CCC6(CCN(c7ccc([C@@H]8c9ccc(O)cc9CC[C@@H]8c8ccccc8)cc7)CC6)O5)CCN43)C2=O)C(=O)N1] | (3S)-3-((5aR)-7-((8-(4-((1R,2S)-6- hydroxy-2-phenyl-1,2,3,4- tetrahydronaphthalen-1-yl)phenyl)-1-oxa- 8-azaspiro[4.5]decan-2-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B177 | 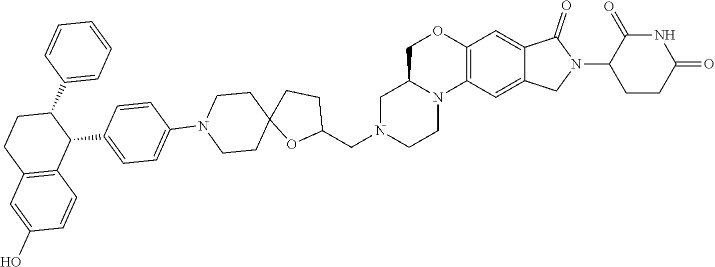 [SMILES: O=C1CCC(N2Cc3cc4c(cc3C2=O)OC[C@@H]2CN(CC3CCC5(CCN(c6ccc([C@@H]7c8ccc(O)cc8CC[C@@H]7c7ccccc7)cc6)CC5)O3)CCN42)C(=O)N1] | 3-((4aS)-3-((8-(4-((1R,2S)-6-hydroxy-2- phenyl-1,2,3,4-tetrahydronaphthalen-1- yl)phenyl)-1-oxa-8-azaspiro[4.5]decan-2- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B178 | 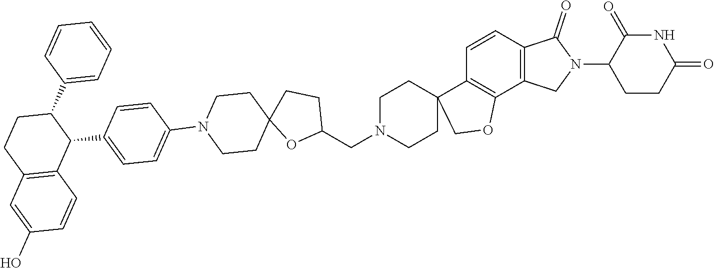 [SMILES: O=C1CCC(N2Cc3c(ccc4c3OCC43CCN(CC4CCC5(CCN(c6ccc([C@@H]7c8ccc(O)cc8CC[C@@H]7c7ccccc7)cc6)CC5)O4)CC3)C2=O)C(=O)N1] | 3-(1′-((8-(4-((1R,2S)-6-hydroxy-2-phenyl- 1,2,3,4-tetrahydronaphthalen-1-yl)phenyl)- 1-oxa-8-azaspiro[4.5]decan-2-yl)methyl)- 6-oxo-6,8-dihydro-2H,7H-spiro[furo[2,3- e]isoindole-3,4′-piperidin]-7-yl)piperidine- 2,6-dione |
| B204 | 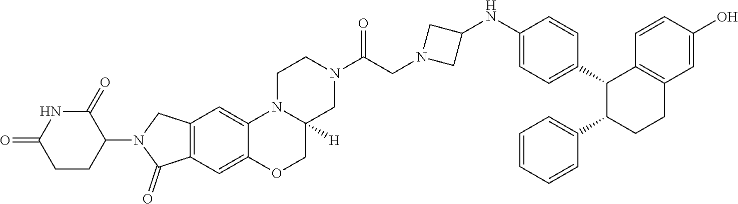 [SMILES: O=C1CCC(N2Cc3cc4c(cc3C2=O)OC[C@@H]2CN(C(=O)CN3CC(Nc5ccc([C@@H]6c7ccc(O)cc7CC[C@@H]6c6ccccc6)cc5)C3)CCN42)C(=O)N1] | 3-((S)-3-(2-(3-((4-((1R,2S)-6-hydroxy-2- phenyl-1,2,3,4-tetrahydronaphthalen-1- yl)phenyl)amino)azetidin-1-yl)acetyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| B205 | 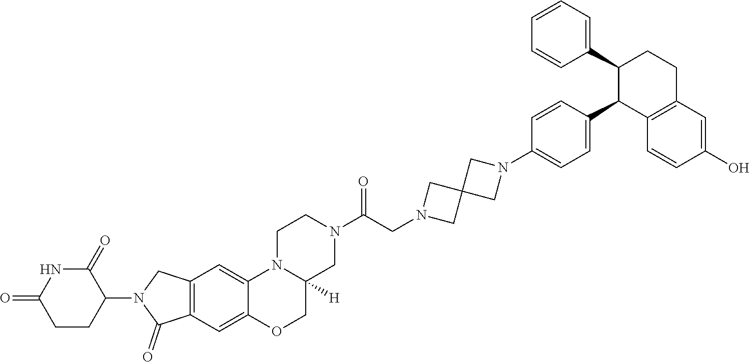 [SMILES: O=C1CCC(N2Cc3cc4c(cc3C2=O)OC[C@@H]2CN(C(=O)CN3CC5(C3)CN(c3ccc([C@@H]6c7ccc(O)cc7CC[C@@H]6c6ccccc6)cc3)C5)CCN42)C(=O)N1] | 3-((S)-3-(2-(6-(4-((1R,2S)-6-hydroxy-2- phenyl-1,2,3,4-tctrahydronaphthalen-1- yl)phenyl)-2,6-diazaspiro[3.3]heptan-2- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B206 | 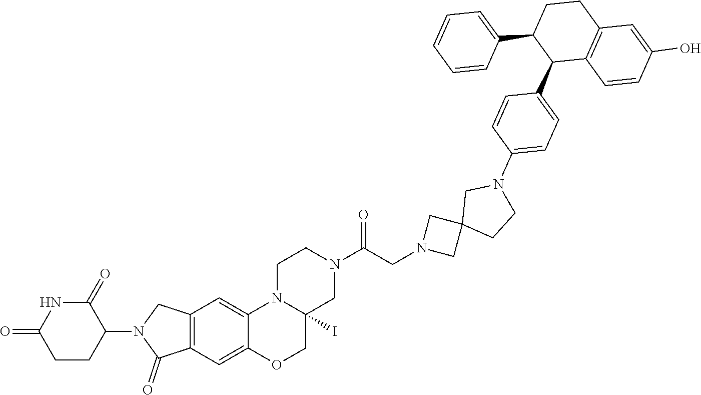 [SMILES: O=C1CCC(N2Cc3cc4c(cc3C2=O)OC[C@]2(I)CN(C(=O)CN3CC5(CCN(c6ccc([C@@H]7c8ccc(O)cc8CC[C@@H]7c7ccccc7)cc6)C5)C3)CCN42)C(=O)N1] | 3-((S)-3-(2-(6-(4-((1R,2S)-6-hydroxy-2- phenyl-1,2,3,4-tetrahydronaphthalen-1- yl)phenyl)-2,6-diazaspiro[3.4]octan-2- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B207 | 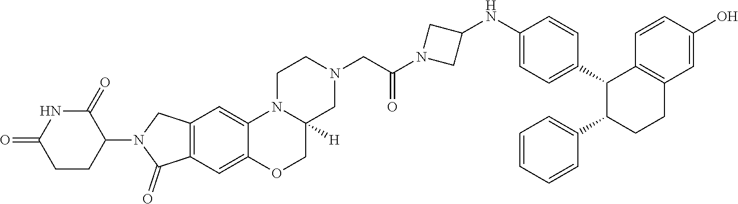 [SMILES: O=C1CCC(N2Cc3cc4c(cc3C2=O)OC[C@@H]2CN(CC(=O)N3CC(Nc5ccc([C@@H]6c7ccc(O)cc7CC[C@@H]6c6ccccc6)cc5)C3)CCN42)C(=O)N1] | 3-((S)-3-(2-(3-((4-((1R,2S)-6-hydroxy-2- phenyl-1,2,3,4-tetrahydronaphthalen-1- yl)phenyl)amino)azetidin-1-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B208 | 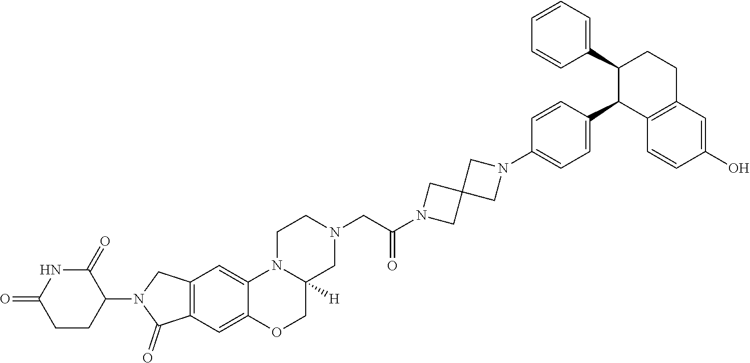 [SMILES: O=C1CCC(N2Cc3cc4c(cc3C2=O)OC[C@@H]2CN(CC(=O)N3CC5(C3)CN(c3ccc([C@@H]6c7ccc(O)cc7CC[C@@H]6c6ccccc6)cc3)C5)CCN42)C(=O)N1] | 3-((S)-3-(2-(6-(4-((1R,2S)-6-hydroxy-2- phenyl-1,2,3,4-tetrahydronaphthalen-1- yl)phenyl)-2,6-diazaspiro[3.3]heptan-2- yl)-2-oxoethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B209 | 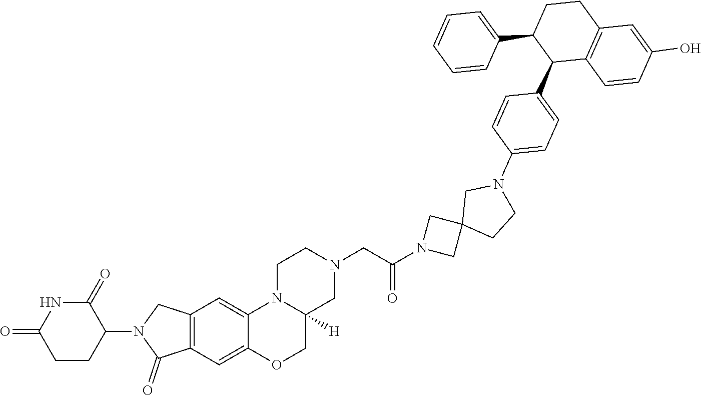 [SMILES: O=C1CCC(N2Cc3cc4c(cc3C2=O)OC[C@@H]2CN(CC(=O)N3CC5(CCN(c6ccc([C@@H]7c8ccc(O)cc8CC[C@@H]7c7ccccc7)cc6)C5)C3)CCN42)C(=O)N1] | 3-((S)-3-(2-(6-(4-((1R,2S)-6-hydroxy-2- phenyl-1,2,3,4-tetrahydronaphthalen-1- yl)phenyl)-2,6-diazaspiro[3.4]octan-2-yl)- 2-oxoethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B211 | 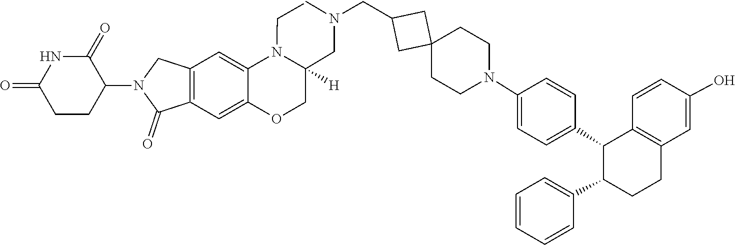 [SMILES: CCN1c2cc3c(cc2OC[C@@H]1CN(C)CC1CC2(CCN(c4ccc([C@@H]5c6ccc(O)cc6CC[C@@H]5c5ccccc5)cc4)CC2)C1)C(=O)N(C1CCC(=O)NC1=O)C3] | 3-((S)-3-((7-(4-((1R,2S)-6-hydroxy-2- phenyl-1,2,3,4-tetrahydronaphthalen-1- yl)phenyl)-7-azaspiro[3.5]nonan-2- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B212 | 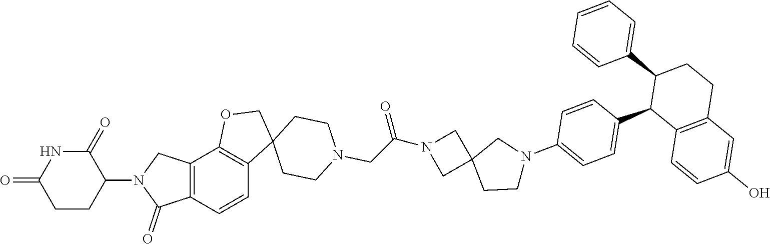 [SMILES: O=C1CCC(N2Cc3c(ccc4c3OCC43CCN(CC(=O)N4CC5(CCN(c6ccc([C@@H]7c8ccc(O)cc8CC[C@@H]7c7ccccc7)cc6)C5)C4)CC3)C2=O)C(=O)N1] | 3-(1′-(2-(6-(4-((1R,2S)-6-hydroxy-2- phenyl-1,2,3,4-tetrahydronaphthalen-1- yl)phenyl)-2,6-diazaspiro[3.4]octan-2-yl)- 2-oxoethyl)-6-oxo-6,8-dihydro-2H,7H- spiro[furo[2,3-e]isoindole-3,4′-piperidin]- 7-yl)piperidine-2,6-dione |
| B213 | 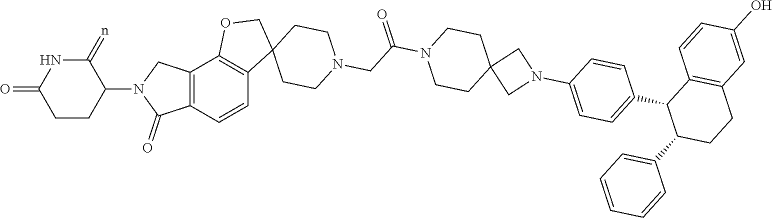 | 3-(1′-(2-(2-(4-((1R,2S)-6-hydroxy-2- phenyl-1,2,3,4-tetrahydronaphthalen-1- yl)phenyl)-2,7-diazaspiro[3.5]nonan-7-yl)- 2-oxoethyl)-6-oxo-6,8-dihydro-2H,7H- spiro[furo[2,3-e]isoindole-3,4′-piperidin]- 7-yl)piperidine-2,6-dione |
| B214 | 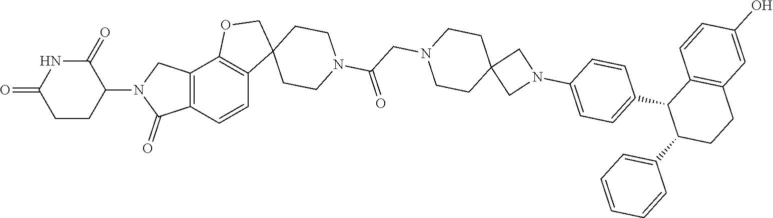 [SMILES: O=C1CCC(N2Cc3c(ccc4c3OCC43CCN(C(=O)CN4CCC5(CC4)CN(c4ccc([C@@H]6c7ccc(O)cc7CC[C@@H]6c6ccccc6)cc4)C5)CC3)C2=O)C(=O)N1] | 3-(1′-(2-(2-(4-((1R,2S)-6-hydroxy-2- phenyl-1,2,3,4-tetrahydronaphthalen-1- yl)phenyl)-2,7-diazaspiro[3.5]nonan-7- yl)acctyl)-6-oxo-6,8-dihydro-2H,7H- spiro[furo[2,3-e]isoindole-3,4′-piperidin]- 7-yl)piperidine-2,6-dione |
| B215 | 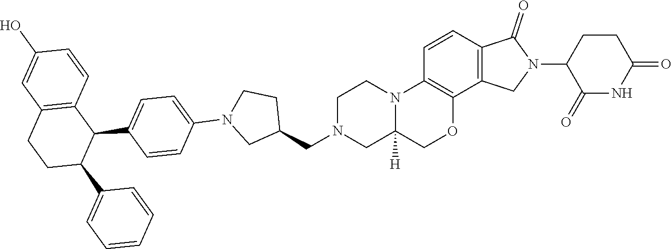 [SMILES: O=C1CCC(N2Cc3c(ccc4c3OC[C@H]3CN(C[C@@H]5CCN(c6ccc([C@@H]7c8ccc(O)cc8CC[C@@H]7c7ccccc7)cc6)C5)CCN43)C2=O)C(=O)N1] | 3-((R)-7-(((S)-1-(4-((1R,2S)-6-hydroxy-2- phenyl-1,2,3,4-tetrahydronaphthalen-1- yl)phenyl)pyrrolidin-3-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B216 | 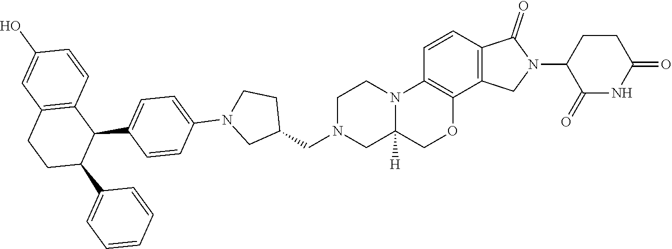 [SMILES: O=C1CCC(N2Cc3c(ccc4c3OC[C@H]3CN(C[C@H]5CCN(c6ccc([C@@H]7c8ccc(O)cc8CC[C@@H]7c7ccccc7)cc6)C5)CCN43)C2=O)C(=O)N1] | 3-((R)-7-(((R)-1-(4-((1R,2S)-6-hydroxy-2- phenyl-1,2,3,4-tetrahydronaphthalen-1- yl)phenyl)pyrrolidin-3-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B218 | 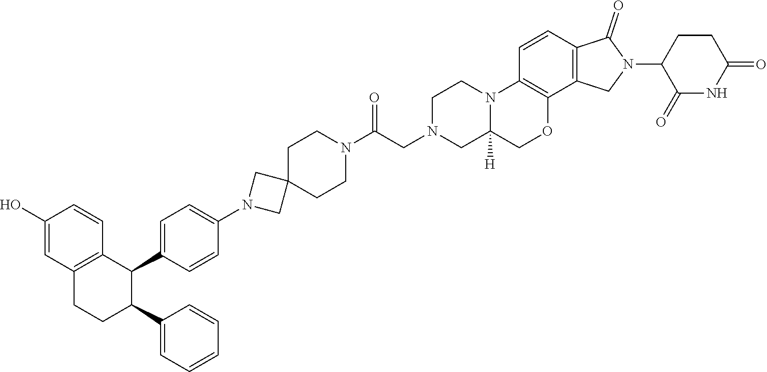 [SMILES: O=C1CCC(N2Cc3c(ccc4c3OC[C@H]3CN(CC(=O)N5CCC6(CC5)CN(c5ccc([C@@H]7c8ccc(O)cc8CC[C@@H]7c7ccccc7)cc5)C6)CCN43)C2=O)C(=O)N1] | 3-((R)-7-(2-(2-(4-((1R,2S)-6-hydroxy-2- phenyl-1,2,3,4-tetrahydronaphthalen-1- yl)phenyl)-2,7-diazaspiro[3.5]nonan-7-yl)- 2-oxoethyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B219 | 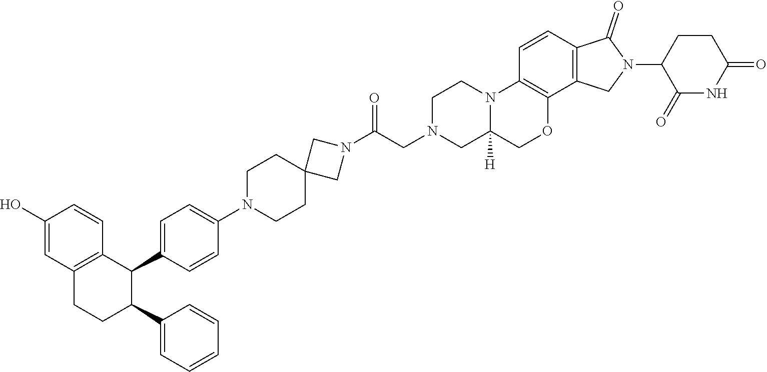 [SMILES: O=C1CCC(N2Cc3c(ccc4c3OC[C@H]3CN(CC(=O)N5CC6(CCN(c7ccc([C@@H]8c9ccc(O)cc9CC[C@@H]8c8ccccc8)cc7)CC6)C5)CCN43)C2=O)C(=O)N1] | 3-((R)-7-(2-(7-(4-((1R,2S)-6-hydroxy-2- phenyl-1,2,3,4-tetrahydronaphthalen-1- yl)phenyl)-2,7-diazaspiro[3.5]nonan-2-yl)- 2-oxoethyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B220 | 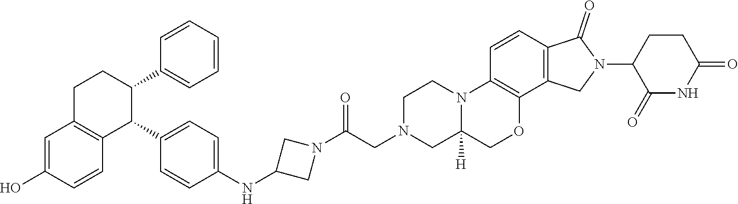 [SMILES: O=C1CCC(N2Cc3c(ccc4c3OC[C@H]3CN(CC(=O)N5CC(Nc6ccc([C@@H]7c8ccc(O)cc8CC[C@@H]7c7ccccc7)cc6)C5)CCN43)C2=O)C(=O)N1] | 3-((R)-7-(2-(3-((4-((1R,2S)-6-hydroxy-2- phenyl-1,2,3,4-tetrahydronaphthalen-1- yl)phenyl)amino)azetidin-1-yl)-2- oxoethyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B221 | 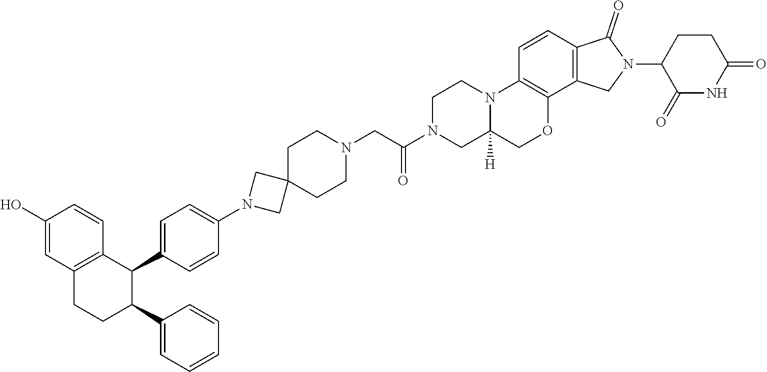 [SMILES: O=C1CCC(N2Cc3c(ccc4c3OC[C@H]3CN(C(=O)CN5CCC6(CC5)CN(c5ccc([C@@H]7c8ccc(O)cc8CC[C@@H]7c7ccccc7)cc5)C6)CCN43)C2=O)C(=O)N1] | 3-((R)-7-(2-(2-(4-((1R,2S)-6-hydroxy-2- phenyl-1,2,3,4-tetrahydronaphthalen-1- yl)phenyl)-2,7-diazaspiro[3.5]nonan-7- yl)acetyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B222 | 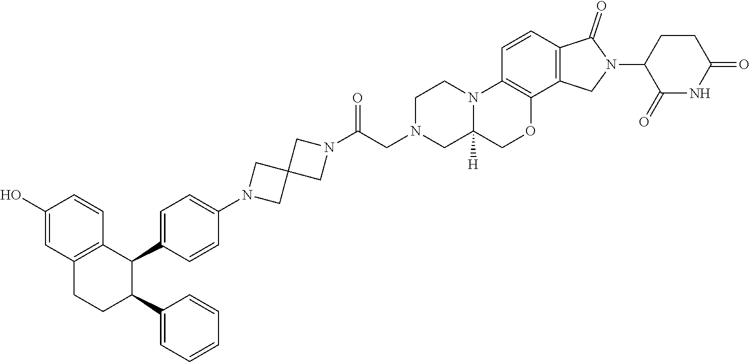 [SMILES: O=C1CCC(N2Cc3c(ccc4c3OC[C@H]3CN(CC(=O)N5CC6(C5)CN(c5ccc([C@@H]7c8ccc(O)cc8CC[C@@H]7c7ccccc7)cc5)C6)CCN43)C2=O)C(=O)N1] | 3-((R)-7-(2-(6-(4-((1R,2S)-6-hydroxy-2- phenyl-1,2,3,4-tetrahydronaphthalen-1- yl)phenyl)-2,6-diazaspiro[3.3]heptan-2- yl)-2-oxoethyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B223 | 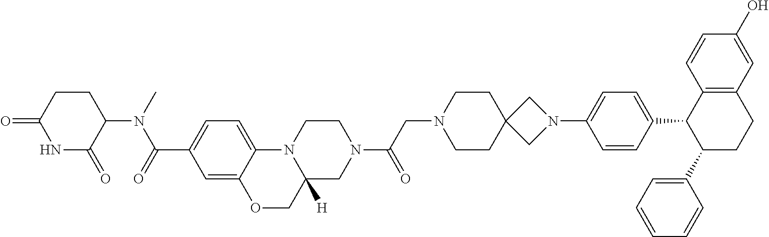 [SMILES: CN(C(=O)c1ccc2c(c1)OC[C@H]1CN(C(=O)CN3CCC4(CC3)CN(c3ccc([C@@H]5c6ccc(O)cc6CC[C@@H]5c5ccccc5)cc3)C4)CCN21)C1CCC(=O)NC1=O] | (4aR)-N-(2,6-dioxopiperidin-3-yl)-3-(2- (2-(4-((1S,2R)-6-hydroxy-2-phenyl- 1,2,3,4-tetrahydronaphthalen-1-yl)phenyl)- 2,7-diazaspiro[3.5]nonan-7-yl)acetyl)-N- methyl-1,2,3,4,4a,5- hexahydrobenzo[b]pyrazino[1,2- d][1,4]oxazine-8-carboxamide |
| B224 | 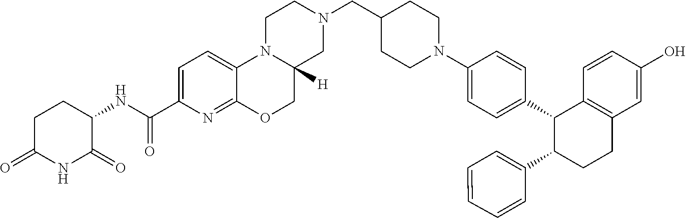 [SMILES: O=C1CC[C@H](NC(=O)c2ccc3c(n2)OC[C@H]2CN(CC4CCN(c5ccc([C@@H]6c7ccc(O)cc7CC[C@@H]6c6ccccc6)cc5)CC4)CCN32)C(=O)N1] | (R)-N-((S)-2,6-dioxopiperidin-3-yl)-3-((1- (4-((1R,2S)-6-hydroxy-2-phenyl-1,2,3,4- tetrahydronaphthalen-1- yl)phenyl)piperidin-4-yl)methyl)- 1,2,3,4,4a,5-hexahydropyrazino[1,2- d]pyrido[2,3-b][1,4]oxazine-8- carboxamide |
| B225 | 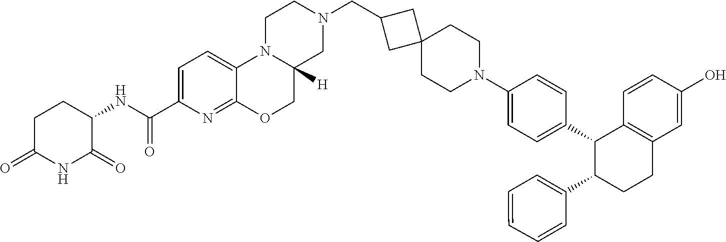 [SMILES: O=C1CC[C@H](NC(=O)c2ccc3c(n2)OC[C@H]2CN(CC4CC5(CCN(c6ccc([C@@H]7c8ccc(O)cc8CC[C@@H]7c7ccccc7)cc6)CC5)C4)CCN32)C(=O)N1] | (R)-N-((S)-2,6-dioxopiperidin-3-yl)-3-((7- (4-((1R,2S)-6-hydroxy-2-phenyl-1,2,3,4- tetrahydronaphthalen-1-yl)phenyl)-7- azaspiro[3.5]nonan-2-yl)methyl)- 1,2,3,4,4a,5-hexahydropyrazino[1,2- d]pyrido[2,3-b][1,4]oxazine-8- carboxamide |
| B226 | 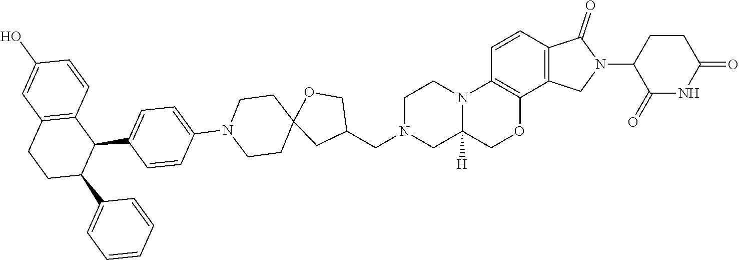 [SMILES: O=C1CCC(N2Cc3c(ccc4c3OC[C@H]3CN(CC5COC6(CCN(c7ccc([C@@H]8c9ccc(O)cc9CC[C@@H]8c8ccccc8)cc7)CC6)C5)CCN43)C2=O)C(=O)N1] | 3-((5aR)-7-((8-(4-((1R,2S)-6-hydroxy-2- phenyl-1,2,3,4-tetrahydronaphthalen-1- yl)phenyl)-1-oxa-8-azaspiro[4.5]decan-3- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B226X | 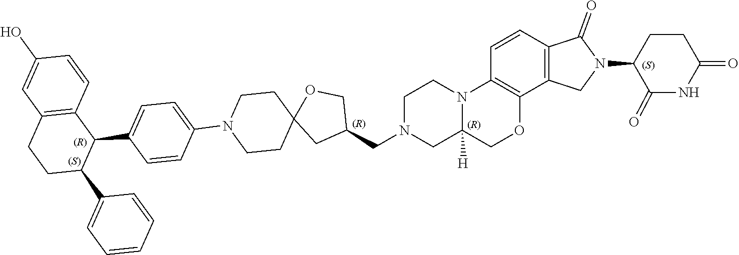 | (S)-3-((R)-7-(((R)-8-(4-((1R,2S)-6- hydroxy-2-phenyl-1,2,3,4- tetrahydronaphthalen-1-yl)phenyl)-1-oxa- 8-azaspiro[4.5]decan-3-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B226Y | 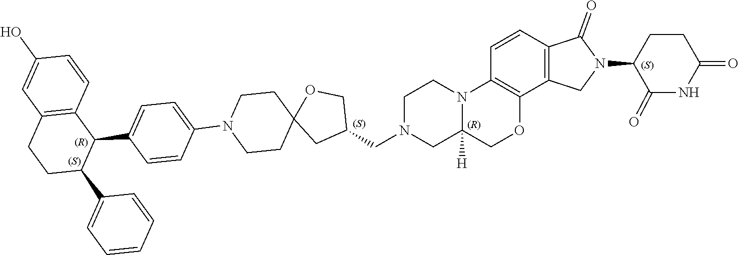 | (S)-3-((R)-7-(((S)-8-(4-((1R,2S)-6- hydroxy-2-phenyl-1,2,3,4- tetrahydronaphthalen-1-yl)phenyl)-1-oxa- 8-azaspiro[4.5]decan-3-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B227 | 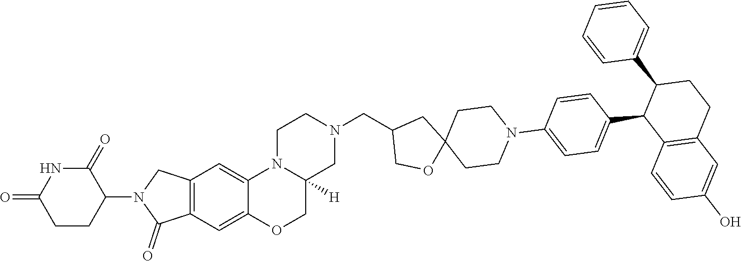 [SMILES: O=C1CCC(N2Cc3cc4c(cc3C2=O)OC[C@@H]2CN(CC3COC5(CCN(c6ccc([C@@H]7c8ccc(O)cc8CC[C@@H]7c7ccccc7)cc6)CC5)C3)CCN42)C(=O)N1] | 3-((4aS)-3-((8-(4-((1R,2S)-6-hydroxy-2- phenyl-1,2,3,4-tetrahydronaphthalen-1- yl)phenyl)-1-oxa-8-azaspiro[4.5]decan-3- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B227X | 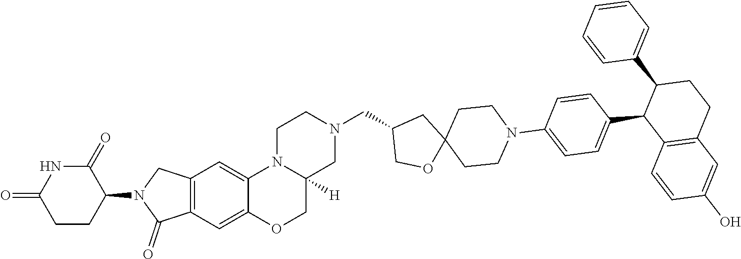 [SMILES: O=C1CC[C@H](N2Cc3cc4c(cc3C2=O)OC[C@@H]2CN(C[C@H]3COC5(CCN(c6ccc([C@@H]7c8ccc(O)cc8CC[C@@H]7c7ccccc7)cc6)CC5)C3)CCN42)C(=O)N1] | (S)-3-((S)-3-(((S)-8-(4-((1R,2S)-6- hydroxy-2-phenyl-1,2,3,4- tetrahydronaphthalen-1-yl)phenyl)-1-oxa- 8-azaspiro[4.5]decan-3-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B227Y | 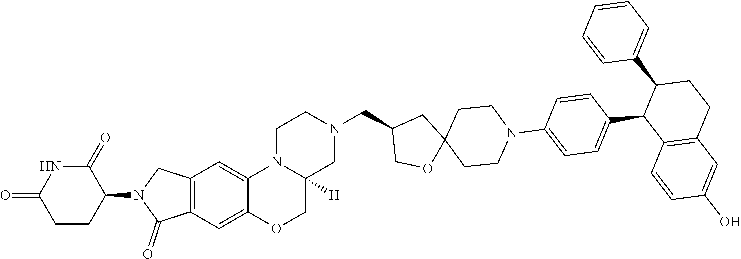 [SMILES: O=C1CC[C@H](N2Cc3cc4c(cc3C2=O)OC[C@@H]2CN(C[C@@H]3COC5(CCN(c6ccc([C@@H]7c8ccc(O)cc8CC[C@@H]7c7ccccc7)cc6)CC5)C3)CCN42)C(=O)N1] | (S)-3-((S)-3-(((R)-8-(4-((1R,2S)-6- hydroxy-2-phenyl-1,2,3,4- tetrahydronaphthalen-1-yl)phenyl)-1-oxa- 8-azaspiro[4.5]decan-3-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B228 | 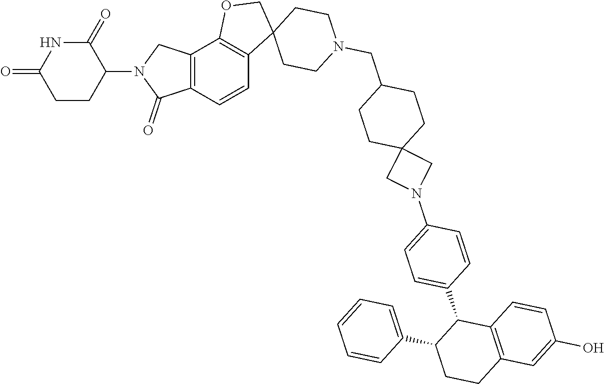 [SMILES: O=C1CCC(N2Cc3c(ccc4c3OCC43CCN(CC4CCC5(CC4)CN(c4ccc([C@@H]6c7ccc(O)cc7CC[C@@H]6c6ccccc6)cc4)C5)CC3)C2=O)C(=O)N1] | 3-(1′-((2-(4-((1R,2S)-6-hydroxy-2-phenyl- 1,2,3,4-tetrahydronaphthalen-1-yl)phenyl)- 2-azaspiro[3.5]nonan-7-yl)methyl)-6-oxo- 6,8-dihydro-2H,7H-spiro[furo[2,3- e]isoindole-3,4′-piperidin]-7-yl)piperidine- 2,6-dione |
| B229 | 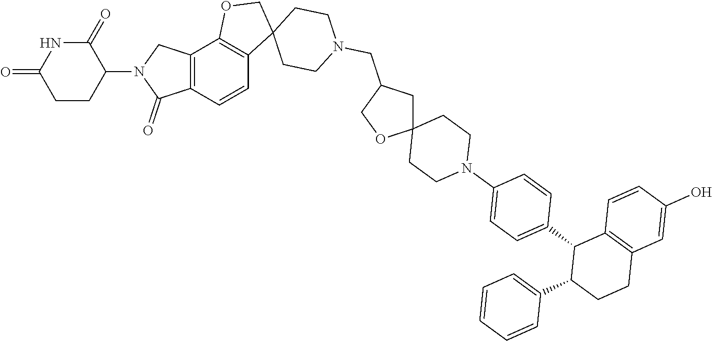 [SMILES: O=C1CCC(N2Cc3c(ccc4c3OCC43CCN(CC4COC5(CCN(c6ccc([C@@H]7c8ccc(O)cc8CC[C@@H]7c7ccccc7)cc6)CC5)C4)CC3)C2=O)C(=O)N1] | 3-(1′-((8-(4-((1R,2S)-6-hydroxy-2-phenyl- 1,2,3,4-tetrahydronaphthalen-1-yl)phenyl)- 1-oxa-8-azaspiro[4.5]decan-3-yl)methyl)- 6-oxo-6,8-dihydro-2H,7H-spiro[furo[2,3- e]isoindole-3,4′-piperidin]-7-yl)piperidine- 2,6-dione |
| B229X | 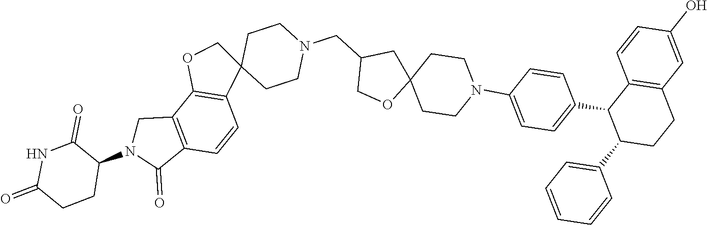 [SMILES: O=C1CC[C@H](N2Cc3c(ccc4c3OCC43CCN(CC4COC5(CCN(c6ccc([C@@H]7c8ccc(O)cc8CC[C@@H]7c7ccccc7)cc6)CC5)C4)CC3)C2=O)C(=O)N1] | (3S)-3-(1′-((8-(4-((1R,2S)-6-hydroxy-2- phenyl-1,2,3,4-tetrahydronaphthalen-1- yl)phenyl)-1-oxa-8-azaspiro[4.5]decan-3- yl)methyl)-6-oxo-6,8-dihydro-2H,7H- spiro[furo[2,3-e]isoindole-3,4′-piperidin]- 7-yl)piperidine-2,6-dione |
| B230 | 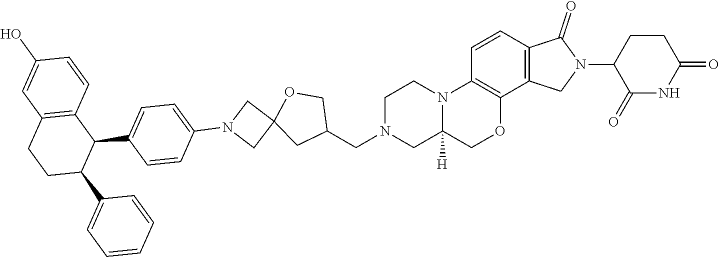 [SMILES: O=C1CCC(N2Cc3c(ccc4c3OC[C@H]3CN(CC5COC6(C5)CN(c5ccc([C@@H]7c8ccc(O)cc8CC[C@@H]7c7ccccc7)cc5)C6)CCN43)C2=O)C(=O)N1] | 3-((5aR)-7-((2-(4-((1R,2S)-6-hydroxy-2- phenyl-1,2,3,4-tetrahydronaphthalen-1- yl)phenyl)-5-oxa-2-azaspiro[3.4]octan-7- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B231 | 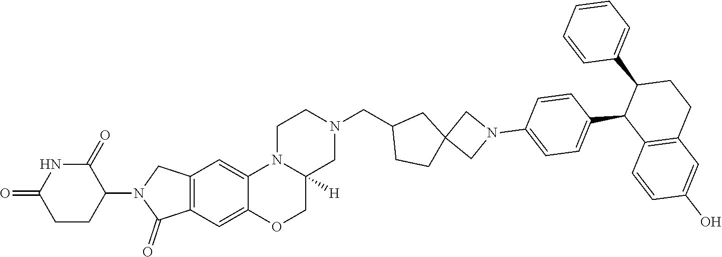 [SMILES: O=C1CCC(N2Cc3cc4c(cc3C2=O)OC[C@@H]2CN(CC3CCC5(C3)CN(c3ccc([C@@H]6c7ccc(O)cc7CC[C@@H]6c6ccccc6)cc3)C5)CCN42)C(=O)N1] | 3-((4aS)-3-((2-(4-((1R,2S)-6-hydroxy-2- phenyl-1,2,3,4-tetrahydronaphthalen-1- yl)phenyl)-5-oxa-2-azaspiro[3.4]octan-7- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B232 | 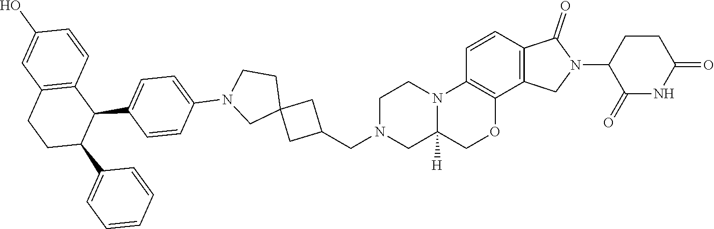 [SMILES: O=C1CCC(N2Cc3c(ccc4c3OC[C@H]3CN(CC5CC6(CCN(c7ccc([C@@H]8c9ccc(O)cc9CC[C@@H]8c8ccccc8)cc7)C6)C5)CCN43)C2=O)C(=O)N1] | 3-((R)-7-((6-(4-((1R,2S)-6-hydroxy-2- phenyl-1,2,3,4-tetrahydronaphthalen-1- yl)phenyl)-6-azaspiro[3.4]octan-2- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B233 | 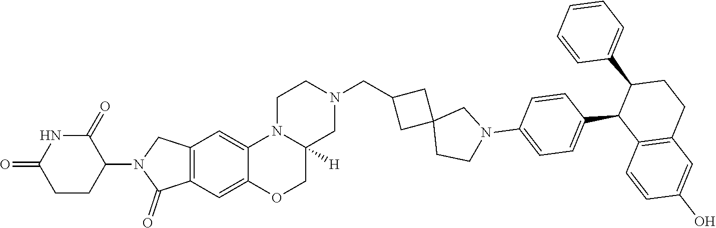 [SMILES: O=C1CCC(N2Cc3cc4c(cc3C2=O)OC[C@@H]2CN(CC3CC5(CCN(c6ccc([C@@H]7c8ccc(O)cc8CC[C@@H]7c7ccccc7)cc6)C5)C3)CCN42)C(=O)N1] | 3-((S)-3-((6-(4-((1R,2S)-6-hydroxy-2- phenyl-1,2,3,4-tetrahydronaphthalen-1- yl)phenyl)-6-azaspiro[3.4]octan-2- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B234 | 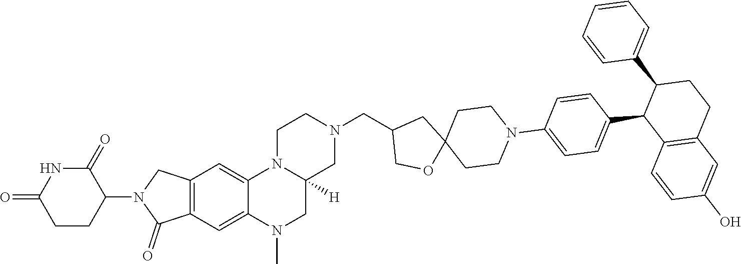 [SMILES: CN1C[C@@H]2CN(CC3COC4(CCN(c5ccc([C@@H]6c7ccc(O)cc7CC[C@@H]6c6ccccc6)cc5)CC4)C3)CCN2c2cc3c(cc21)C(=O)N(C1CCC(=O)NC1=O)C3] | 3-((4aS)-3-((8-(4-((1R,2S)-6-hydroxy-2- phenyl-1,2,3,4-tetrahydronaphthalen-1- yl)phenyl)-1-oxa-8-azaspiro[4.5]decan-3- yl)methyl)-6-methyl-8-oxo- 2,3,4,4a,5,6,8,10-octahydropyrazino[1,2- a]pyrrolo[3,4-g]quinoxalin-9(1H)- yl)piperidine-2,6-dione |
| B236 | 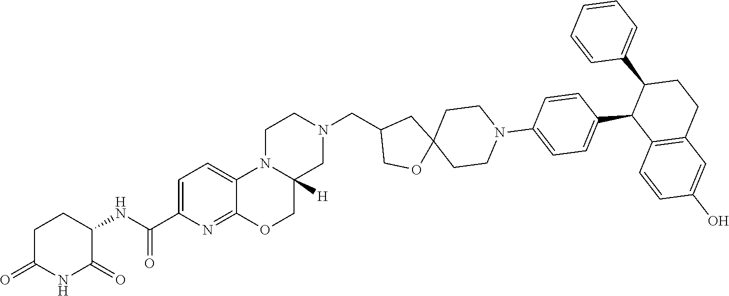 [SMILES: O=C1CC[C@H](NC(=O)c2ccc3c(n2)OC[C@H]2CN(CC4COC5(CCN(c6ccc([C@@H]7c8ccc(O)cc8CC[C@@H]7c7ccccc7)cc6)CC5)C4)CCN32)C(=O)N1] | (4aR)-N-((S)-2,6-dioxopiperidin-3-yl)-3- ((8-(4-((1R,2S)-6-hydroxy-2-phenyl- 1,2,3,4-tetrahydronaphthalen-1-yl)phenyl)- 1-oxa-8-azaspiro[4.5]decan-3-yl)methyl)- 1,2,3,4,4a,5-hexahydropyrazino[1,2- d]pyrido[2,3-b][1,4]oxazine-8- carboxamide |
| B237 | 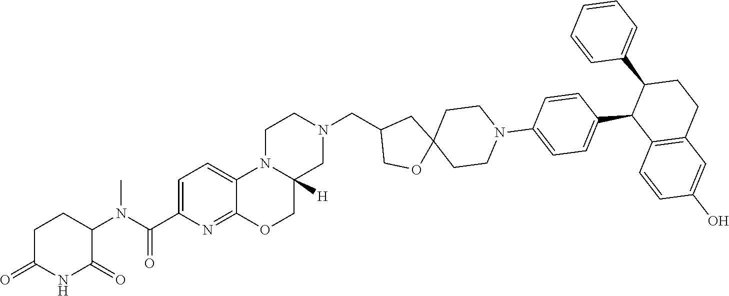 [SMILES: CN(C(=O)c1ccc2c(n1)OC[C@H]1CN(CC3COC4(CCN(c5ccc([C@@H]6c7ccc(O)cc7CC[C@@H]6c6ccccc6)cc5)CC4)C3)CCN21)C1CCC(=O)NC1=O] | (4aR)-N-(2,6-dioxopiperidin-3-yl)-3-((8- (4-((1R,2S)-6-hydroxy-2-phenyl-1,2,3,4- tetrahydronaphthalen-1-yl)phenyl)-1-oxa- 8-azaspiro[4.5]decan-3-yl)methyl)-N- methyl-1,2,3,4,4a,5- hexahydropyrazino[1,2-d]pyrido[2,3- b][1,4]oxazine-8-carboxamide |
| B238 | 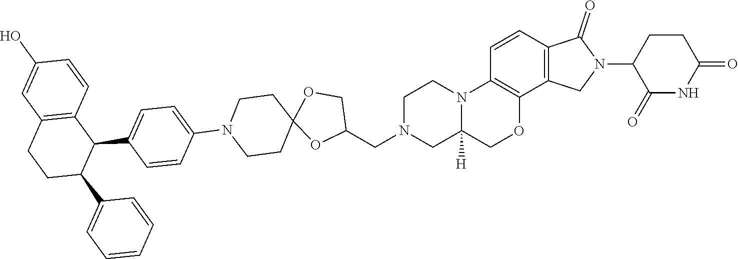 [SMILES: O=C1CCC(N2Cc3c(ccc4c3OC[C@H]3CN(CC5COC6(CCN(c7ccc([C@@H]8c9ccc(O)cc9CC[C@@H]8c8ccccc8)cc7)CC6)O5)CCN43)C2=O)C(=O)N1] | 3-((5aR)-7-((8-(4-((1R,2S)-6-hydroxy-2- phenyl-1,2,3,4-tetrahydronaphthalen-1- yl)phenyl)-1,4-dioxa-8- azaspiro[4.5]decan-2-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B239 | 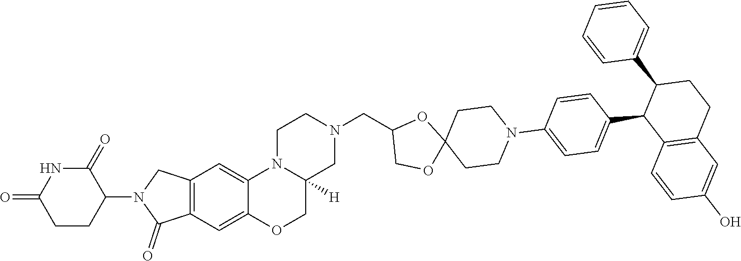 [SMILES: O=C1CCC(N2Cc3cc4c(cc3C2=O)OC[C@@H]2CN(CC3COC5(CCN(c6ccc([C@@H]7c8ccc(O)cc8CC[C@@H]7c7ccccc7)cc6)CC5)O3)CCN42)C(=O)N1] | 3-((4aS)-3-((8-(4-((1R,2S)-6-hydroxy-2- phenyl-1,2,3,4-tetrahydronaphthalen-1- yl)phenyl)-1,4-dioxa-8- azaspiro[4.5]decan-2-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B240 | 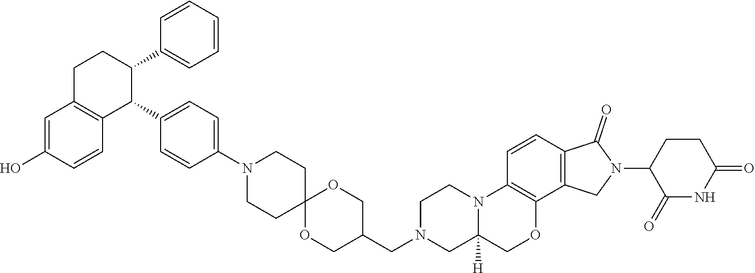 [SMILES: O=C1CCC(N2Cc3c(ccc4c3OC[C@H]3CN(CC5COC6(CCN(c7ccc([C@@H]8c9ccc(O)cc9CC[C@@H]8c8ccccc8)cc7)CC6)OC5)CCN43)C2=O)C(=O)N1] | 3-((R)-7-((9-(4-((1R,2S)-6-hydroxy-2- phenyl-1,2,3,4-tetrahydronaphthalen-1- yl)phenyl)-1,5-dioxa-9- azaspiro[5.5]undecan-3-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B242 | 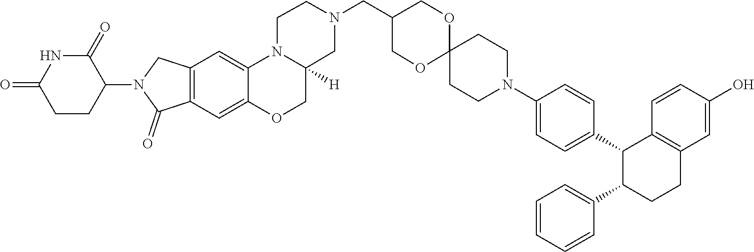 [SMILES: O=C1CCC(N2Cc3cc4c(cc3C2=O)OC[C@@H]2CN(CC3COC5(CCN(c6ccc([C@@H]7c8ccc(O)cc8CC[C@@H]7c7ccccc7)cc6)CC5)OC3)CCN42)C(=O)N1] | 3-((S)-3-((9-(4-((1R,2S)-6-hydroxy-2- phenyl-1,2,3,4-tetrahydronaphthalen-1- yl)phenyl)-1,5-dioxa-9- azaspiro[5.5]undecan-3-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B243 | 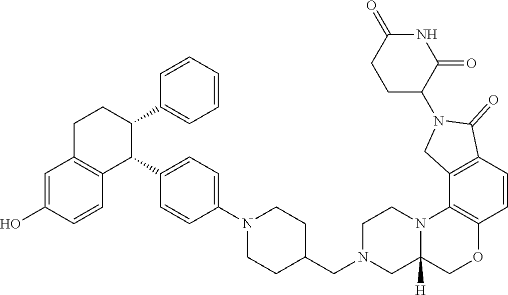 [SMILES: O=C1CCC(N2Cc3c(ccc4c3N3CCN(CC5CCN(c6ccc([C@@H]7c8ccc(O)cc8CC[C@@H]7c7ccccc7)cc6)CC5)C[C@H]3CO4)C2=O)C(=O)N1] | 3-((S)-9-((1-(4-((1R,2S)-6-hydroxy-2- phenyl-1,2,3,4-tetrahydronaphthalen-1- yl)phenyl)piperidin-4-yl)methyl)-3-oxo- 1,3,7,7a,8,9,10,11-octahydro-2H- pyrazino[1′,2′:4,5][1,4]oxazino[3,2- e]isoindol-2-yl)piperidine-2,6-dione |
| B244 | 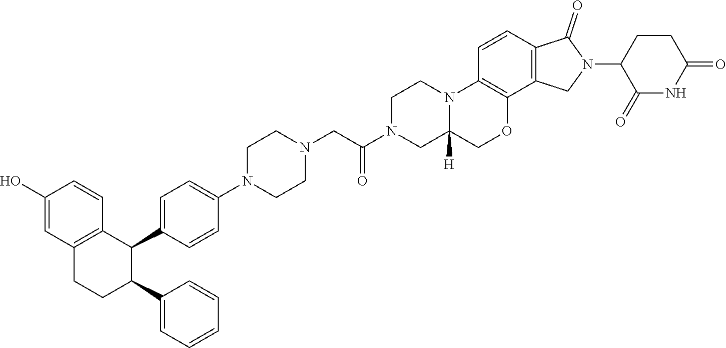 [SMILES: O=C1CCC(N2Cc3c(ccc4c3OC[C@@H]3CN(C(=O)CN5CCN(c6ccc([C@@H]7c8ccc(O)cc8CC[C@@H]7c7ccccc7)cc6)CC5)CCN43)C2=O)C(=O)N1] | 3-((S)-7-(2-(4-(4-((1R,2S)-6-hydroxy-2- phenyl-1,2,3,4-tetrahydronaphthalen-1- yl)phenyl)piperazin-1-yl)acetyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B245 | 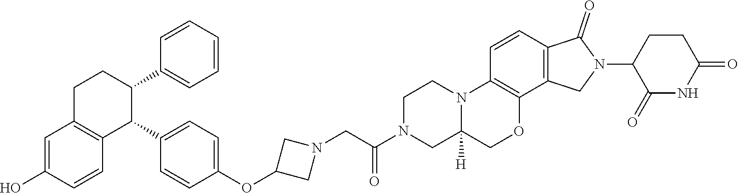 [SMILES: O=C1CCC(N2Cc3c(ccc4c3OC[C@H]3CN(C(=O)CN5CC(Oc6ccc([C@@H]7c8ccc(O)cc8CC[C@@H]7c7ccccc7)cc6)C5)CCN43)C2=O)C(=O)N1] | 3-((R)-7-(2-(3-(4-((1R,2S)-6-hydroxy-2- phenyl-1,2,3,4-tetrahydronaphthalen-1- yl)phenoxy)azetidin-1-yl)acetyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B246 | 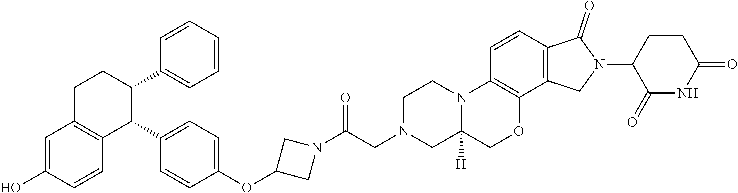 [SMILES: O=C1CCC(N2Cc3c(ccc4c3OC[C@H]3CN(CC(=O)N5CC(Oc6ccc([C@@H]7c8ccc(O)cc8CC[C@@H]7c7ccccc7)cc6)C5)CCN43)C2=O)C(=O)N1] | 3-((R)-7-(2-(3-(4-((1R,2S)-6-hydroxy-2- phenyl-1,2,3,4-tetrahydronaphthalen-1- yl)phenoxy)azetidin-1-yl)-2-oxoethyl)-1- oxo-1,3,5,5a,6,7,8,9-octahydro-2H- pyrazino[1′,2′:4,5][1,4]oxazino[2,3- elisoindol-2-yl)piperidine-2,6-dione |
| B247 | 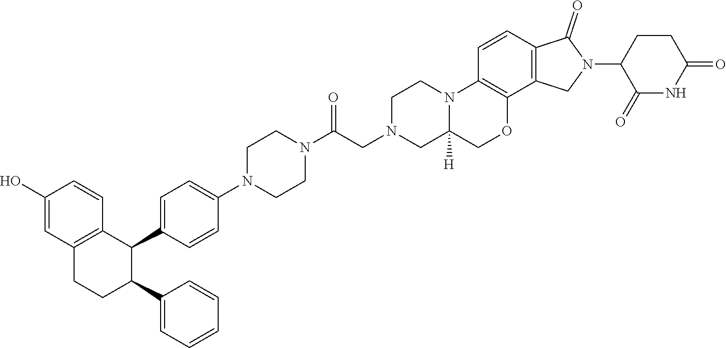 [SMILES: O=C1CCC(N2Cc3c(ccc4c3OC[C@H]3CN(CC(=O)N5CCN(c6ccc([C@@H]7c8ccc(O)cc8CC[C@@H]7c7ccccc7)cc6)CC5)CCN43)C2=O)C(=O)N1] | 3-((R)-7-(2-(4-(4-((1R,2S)-6-hydroxy-2- phenyl-1,2,3,4-tetrahydronaphthalen-1- yl)phenyl)piperazin-1-yl)-2-oxoethyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| B248 | 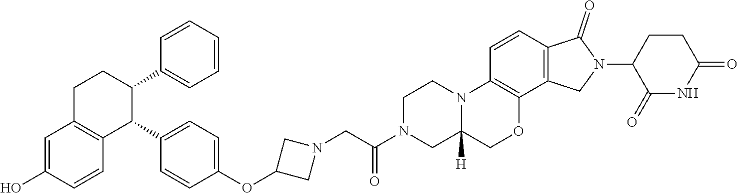 [SMILES: O=C1CCC(N2Cc3c(ccc4c3OC[C@@H]3CN(C(=O)CN5CC(Oc6ccc([C@@H]7c8ccc(O)cc8CC[C@@H]7c7ccccc7)cc6)C5)CCN43)C2=O)C(=O)N1] | 3-((S)-7-(2-(3-(4-((1R,2S)-6-hydroxy-2- phenyl-1,2,3,4-tetrahydronaphthalen-1- yl)phenoxy)azetidin-1-yl)acetyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B249 | 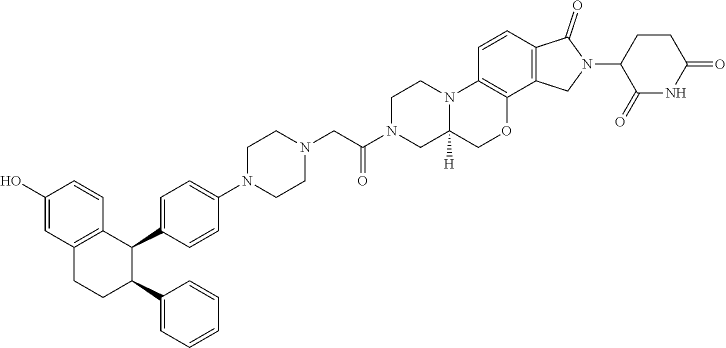 [SMILES: O=C1CCC(N2Cc3c(ccc4c3OC[C@H]3CN(C(=O)CN5CCN(c6ccc([C@@H]7c8ccc(O)cc8CC[C@@H]7c7ccccc7)cc6)CC5)CCN43)C2=O)C(=O)N1] | 3-((R)-7-(2-(4-(4-((1R,2S)-6-hydroxy-2- phenyl-1,2,3,4-tetrahydronaphthalen-1- yl)phenyl)piperazin-1-yl)acetyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B250 | 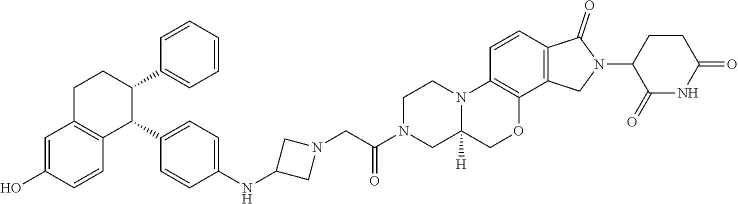 [SMILES: O=C1CCC(N2Cc3c(ccc4c3OC[C@H]3CN(C(=O)CN5CC(Nc6ccc([C@@H]7c8ccc(O)cc8CC[C@@H]7c7ccccc7)cc6)C5)CCN43)C2=O)C(=O)N1] | 3-((R)-7-(2-(3-((4-((1R,2S)-6-hydroxy-2- phenyl-1,2,3,4-tetrahydronaphthalen-1- yl)phenyl)amino)azetidin-1-yl)acetyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| B251 | 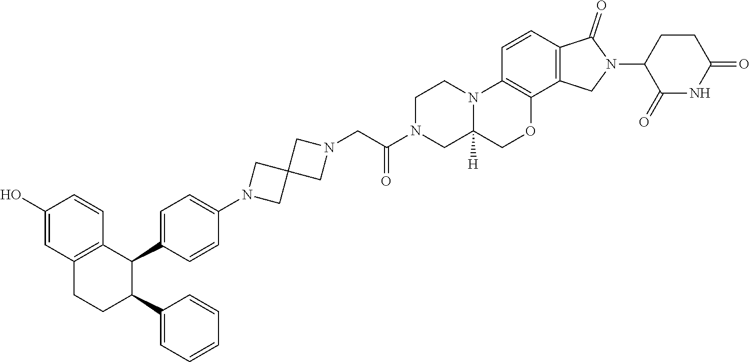 [SMILES: O=C1CCC(N2Cc3c(ccc4c3OC[C@H]3CN(C(=O)CN5CC6(C5)CN(c5ccc([C@@H]7c8ccc(O)cc8CC[C@@H]7c7ccccc7)cc5)C6)CCN43)C2=O)C(=O)N1] | 3-((R)-7-(2-(6-(4-((1R,2S)-6-hydroxy-2- phenyl-1,2,3,4-tetrahydronaphthalen-1- yl)phenyl)-2,6-diazaspiro[3.3]heptan-2- yl)acetyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B252 | 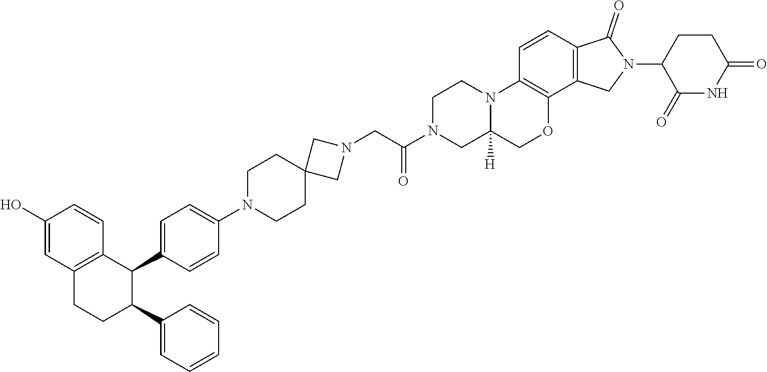 [SMILES: O=C1CCC(N2Cc3c(ccc4c3OC[C@H]3CN(C(=O)CN5CC6(CCN(c7ccc([C@@H]8c9ccc(O)cc9CC[C@@H]8c8ccccc8)cc7)CC6)C5)CCN43)C2=O)C(=O)N1] | 3-((R)-7-(2-(7-(4-((1R,2S)-6-hydroxy-2- phenyl-1,2,3,4-tetrahydronaphthalen-1- yl)phenyl)-2,7-diazaspiro[3.5]nonan-2- yl)acetyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B253 | 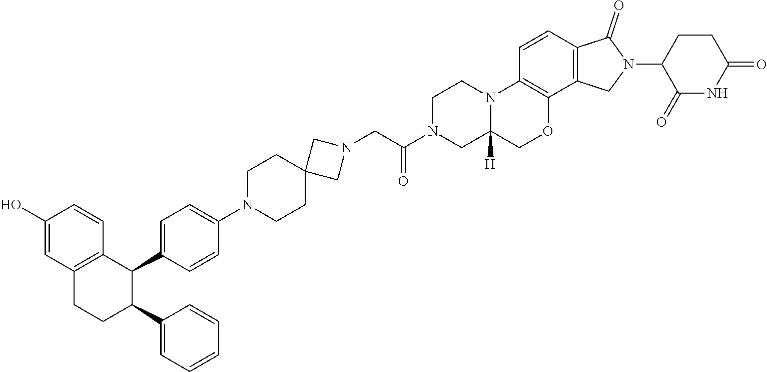 [SMILES: O=C1CCC(N2Cc3c(ccc4c3OC[C@@H]3CN(C(=O)CN5CC6(CCN(c7ccc([C@@H]8c9ccc(O)cc9CC[C@@H]8c8ccccc8)cc7)CC6)C5)CCN43)C2=O)C(=O)N1] | 3-((S)-7-(2-(7-(4-((1R,2S)-6-hydroxy-2- phenyl-1,2,3,4-tetrahydronaphthalen-1- yl)phenyl)-2,7-diazaspiro[3.5]nonan-2- yl)acetyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B254 | 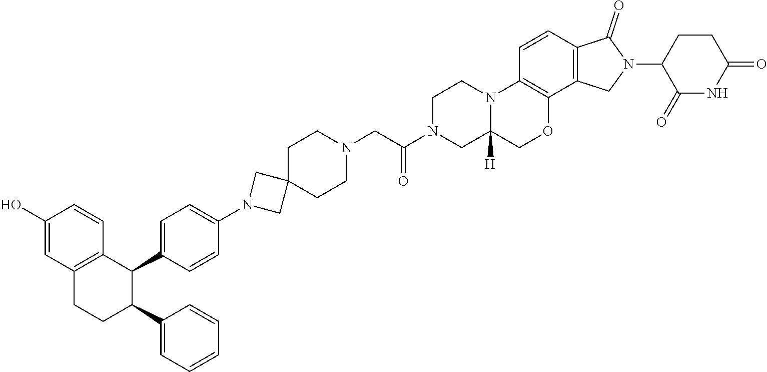 [SMILES: O=C1CCC(N2Cc3c(ccc4c3OC[C@@H]3CN(C(=O)CN5CCC6(CC5)CN(c5ccc([C@@H]7c8ccc(O)cc8CC[C@@H]7c7ccccc7)cc5)C6)CCN43)C2=O)C(=O)N1] | 3-((S)-7-(2-(2-(4-((1R,2S)-6-hydroxy-2- phenyl-1,2,3,4-tctrahydronaphthalen-1- yl)phenyl)-2,7-diazaspiro[3.5]nonan-7- yl)acetyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B255 | 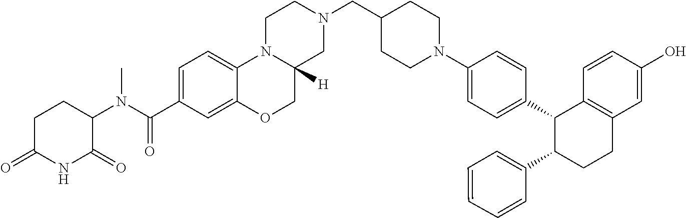 [SMILES: CN(C(=O)c1ccc2c(c1)OC[C@H]1CN(CC3CCN(c4ccc([C@@H]5c6ccc(O)cc6CC[C@@H]5c5ccccc5)cc4)CC3)CCN21)C1CCC(=O)NC1=O] | (4aR)-N-(2,6-dioxopiperidin-3-yl)-3-((1- (4-((1R,2S)-6-hydroxy-2-phenyl-1,2,3,4- tetrahydronaphthalen-1- yl)phenyl)piperidin-4-yl)methyl)-N- methyl-1,2,3,4,4a,5- hexahydrobenzo[b]pyrazino[1,2- d][1,4]oxazine-8-carboxamide |
| B256 | 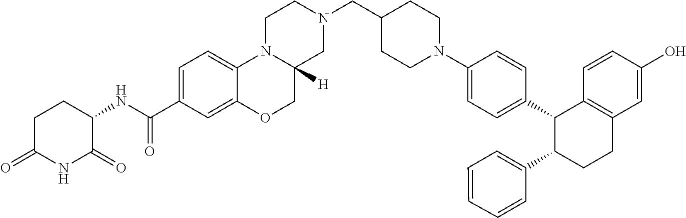 [SMILES: O=C1CC[C@H](NC(=O)c2ccc3c(c2)OC[C@H]2CN(CC4CCN(c5ccc([C@@H]6c7ccc(O)cc7CC[C@@H]6c6ccccc6)cc5)CC4)CCN32)C(=O)N1] | (R)-N-((S)-2,6-dioxopiperidin-3-yl)-3-((1- (4-((1R,2S)-6-hydroxy-2-phenyl-1,2,3,4- tetrahydronaphthalen-1- yl)phenyl)piperidin-4-yl)methyl)- 1,2,3,4,4a,5- hexahydrobenzo[b]pyrazino[1,2- d][1,4]oxazine-8-carboxamide |
| B257 | 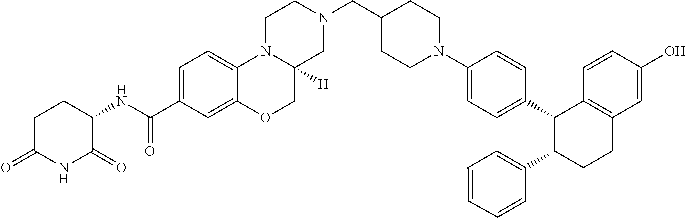 [SMILES: O=C1CC[C@H](NC(=O)c2ccc3c(c2)OC[C@@H]2CN(CC4CCN(c5ccc([C@@H]6c7ccc(O)cc7CC[C@@H]6c6ccccc6)cc5)CC4)CCN32)C(=O)N1] | (S)-N-((S)-2,6-dioxopiperidin-3-yl)-3-((1- (4-((1R,2S)-6-hydroxy-2-phenyl-1,2,3,4- tetrahydronaphthalen-1- yl)phenyl)piperidin-4-yl)methyl)- 1,2,3,4,4a,5- hexahydrobenzo[b]pyrazino[1,2- d][1,4]oxazine-8-carboxamide |
| B258 | 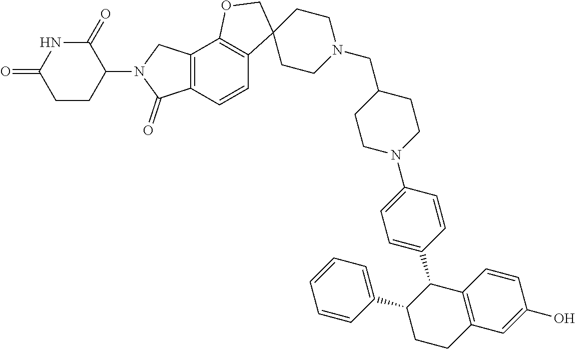 [SMILES: O=C1CCC(N2Cc3c(ccc4c3OCC43CCN(CC4CCN(c5ccc([C@@H]6c7ccc(O)cc7CC[C@@H]6c6ccccc6)cc5)CC4)CC3)C2=O)C(=O)N1] | 3-(1′-((1-(4-((1R,2S)-6-hydroxy-2-phenyl- 1,2,3,4-tetrahydronaphthalen-1- yl)phenyl)piperidin-4-yl)methyl)-6-oxo- 6,8-dihydro-2H,7H-spiro[furo[2,3- e]isoindole-3,4′-piperidin]-7-yl)piperidine- 2,6-dione |
| B295 | 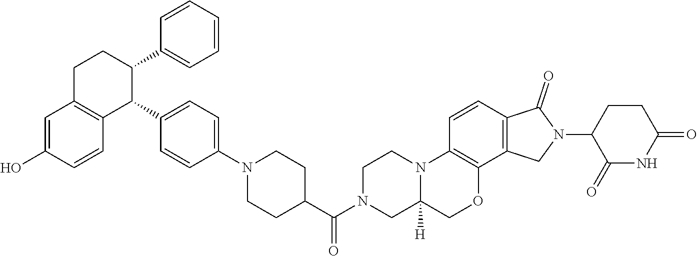 [SMILES: O=C1CCC(N2Cc3c(ccc4c3OC[C@H]3CN(C(=O)C5CCN(c6ccc([C@@H]7c8ccc(O)cc8CC[C@@H]7c7ccccc7)cc6)CC5)CCN43)C2=O)C(=O)N1] | 3-((R)-7-(1-(4-((1R,2S)-6-hydroxy-2- phenyl-1,2,3,4-tetrahydronaphthalen-1- yl)phenyl)piperidine-4-carbonyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B298 | 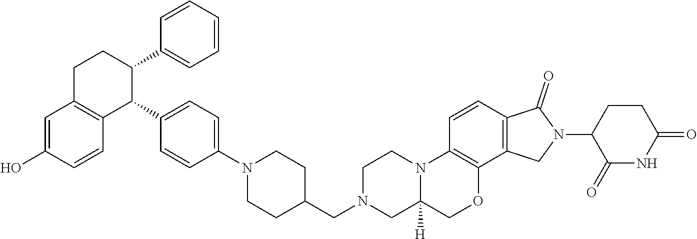 [SMILES: O=C1CCC(N2Cc3c(ccc4c3OC[C@H]3CN(CC5CCN(c6ccc([C@@H]7c8ccc(O)cc8CC[C@@H]7c7ccccc7)cc6)CC5)CCN43)C2=O)C(=O)N1] | 3-((R)-7-((1-(4-((1R,2S)-6-hydroxy-2- phenyl-1,2,3,4-tetrahydronaphthalen-1- yl)phenyl)piperidin-4-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B306 | 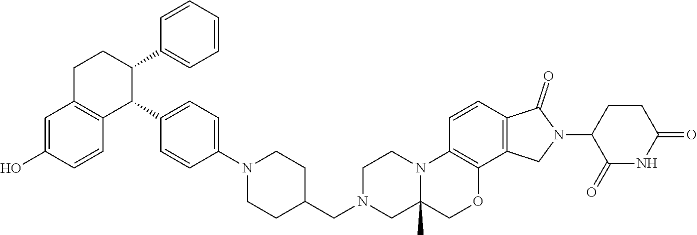 [SMILES: C[C@]12COc3c(ccc4c3CN(C3CCC(=O)NC3=O)C4=O)N1CCN(CC1CCN(c3ccc([C@@H]4c5ccc(O)cc5CC[C@@H]4c4ccccc4)cc3)CC1)C2] | 3-((S)-7-((1-(4-((1R,2S)-6-hydroxy-2- phenyl-1,2,3,4-tetrahydronaphthalen-1- yl)phenyl)piperidin-4-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B306X | 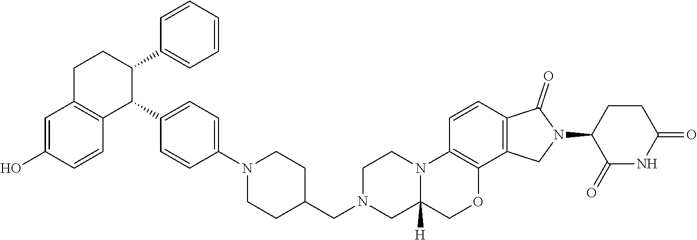 [SMILES: O=C1CC[C@H](N2Cc3c(ccc4c3OC[C@@H]3CN(CC5CCN(c6ccc([C@@H]7c8ccc(O)cc8CC[C@@H]7c7ccccc7)cc6)CC5)CCN43)C2=O)C(=O)N1] | (S)-3-((S)-7-((1-(4-((1R,2S)-6-hydroxy-2- phenyl-1,2,3,4-tetrahydronaphthalen-1- yl)phenyl)piperidin-4-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B307 | 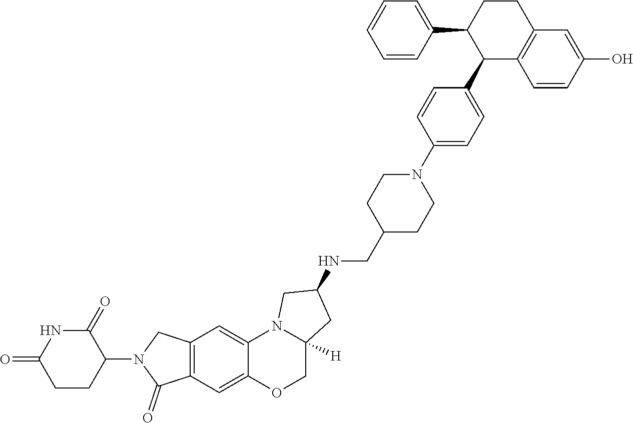 [SMILES: O=C1CCC(N2Cc3cc4c(cc3C2=O)OC[C@@H]2C[C@H](NCC3CCN(c5ccc([C@@H]6c7ccc(O)cc7CC[C@@H]6c6ccccc6)cc5)CC3)CN42)C(=O)N1] | 3-((2S,3aS)-2-(((1-(4-((1R,2S)-6-hydroxy- 2-phenyl-1,2,3,4-tetrahydronaphthalen-1- yl)phenyl)piperidin-4-yl)methyl)amino)-7- oxo-2,3,3a,4,7,9-hexahydro-1H,8H- pyrrolo[1′,2′:4,5][1,4]oxazino[2,3- f]isoindol-8-yl)piperidine-2,6-dione |
| B308 | 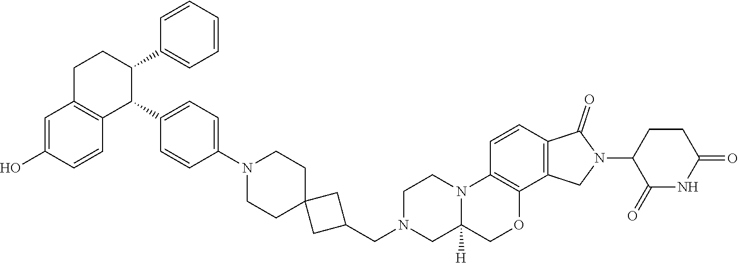 [SMILES: O=C1CCC(N2Cc3c(ccc4c3OC[C@H]3CN(CC5CC6(CCN(c7ccc([C@@H]8c9ccc(O)cc9CC[C@@H]8c8ccccc8)cc7)CC6)C5)CCN43)C2=O)C(=O)N1] | 3-((R)-7-((7-(4-((1R,2S)-6-hydroxy-2- phenyl-1,2,3,4-tetrahydronaphthalen-1- yl)phenyl)-7-azaspiro[3.5]nonan-2- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B308X | 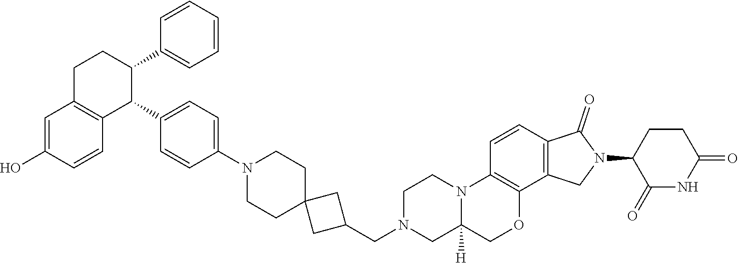 [SMILES: O=C1CC[C@H](N2Cc3c(ccc4c3OC[C@H]3CN(CC5CC6(CCN(c7ccc([C@@H]8c9ccc(O)cc9CC[C@@H]8c8ccccc8)cc7)CC6)C5)CCN43)C2=O)C(=O)N1] | (S)-3-((R)-7-((7-(4-((1R,2S)-6-hydroxy-2- phenyl-1,2,3,4-tetrahydronaphthalen-1- yl)phenyl)-7-azaspiro[3.5]nonan-2- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B309 | 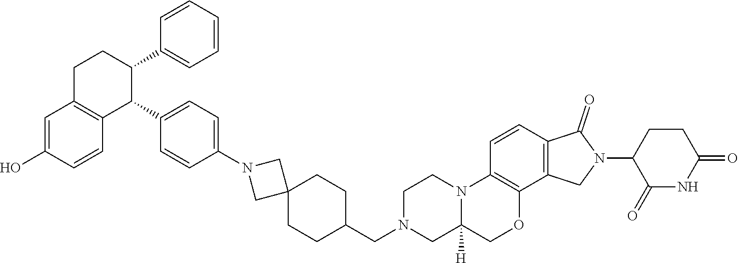 [SMILES: O=C1CCC(N2Cc3c(ccc4c3OC[C@H]3CN(CC5CCC6(CC5)CN(c5ccc([C@@H]7c8ccc(O)cc8CC[C@@H]7c7ccccc7)cc5)C6)CCN43)C2=O)C(=O)N1] | 3-((R)-7-((2-(4-((1R,2S)-6-hydroxy-2- phenyl-1,2,3,4-tetrahydronaphthalen-1- yl)phenyl)-2-azaspiro[3.5]nonan-7- yl)methyl)-1-oxo-1,3,5,5a,6,7,8,9- octahydro-2H- pyrazino[1′,2′:4,5][1,4]oxazino[2,3- c]isoindol-2-yl)piperidine-2,6-dione |
| B310 | 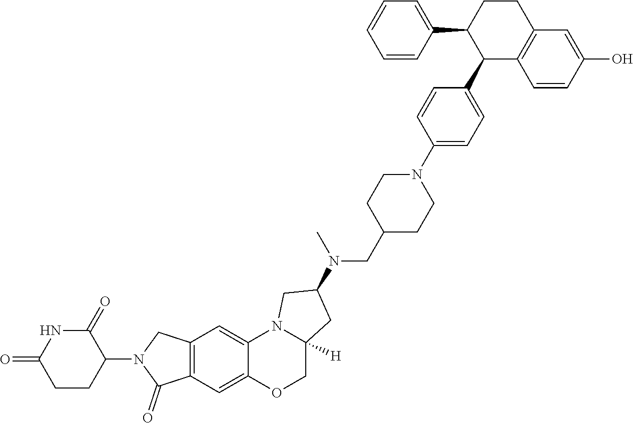 [SMILES: CN(CC1CCN(c2ccc([C@@H]3c4ccc(O)cc4CC[C@@H]3c3ccccc3)cc2)CC1)[C@H]1C[C@H]2COc3cc4c(cc3N2C1)CN(C1CCC(=O)NC1=O)C4=O] | 3-((2S,3aS)-2-(((1-(4-((1R,2S)-6-hydroxy- 2-phenyl-1,2,3,4-tetrahydronaphthalen-1- yl)phenyl)piperidin-4- yl)methyl)(methyl)amino)-7-oxo- 2,3,3a,4,7,9-hexahydro-1H,8H- pyrrolo[1′,2′:4,5][1,4]oxazino[2,3- f]isoindol-8-yl)piperidine-2,6-dione |
| B311 | 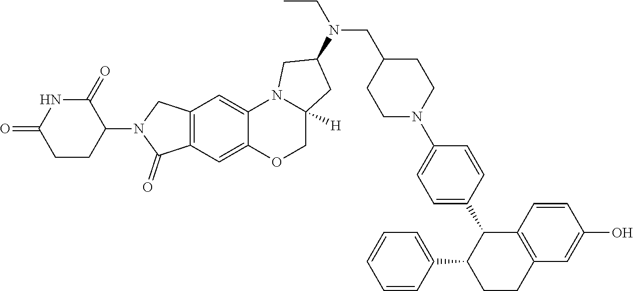 [SMILES: CCN(CC1CCN(c2ccc([C@@H]3c4ccc(O)cc4CC[C@@H]3c3ccccc3)cc2)CC1)[C@H]1C[C@H]2COc3cc4c(cc3N2C1)CN(C1CCC(=O)NC1=O)C4=O] | 3-((2S,3aS)-2-(ethyl((1-(4-((1R,2S)-6- hydroxy-2-phenyl-1,2,3,4- tetrahydronaphthalen-1- yl)phenyl)piperidin-4-yl)methyl)amino)-7- oxo-2,3,3a,4,7,9-hexahydro-1H,8H- pyrrolo[1′,2′:4,5][1,4]oxazino[2,3- f]isoindol-8-yl)piperidine-2,6-dione |
| B312 | 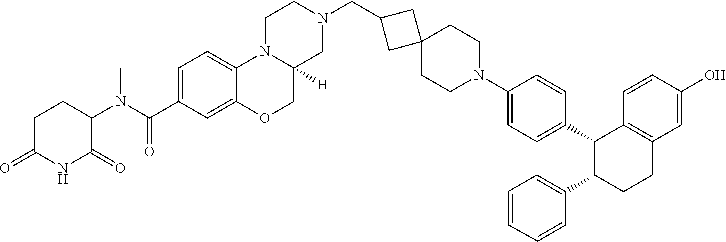 [SMILES: CN(C(=O)c1ccc2c(c1)OC[C@@H]1CN(CC3CC4(CCN(c5ccc([C@@H]6c7ccc(O)cc7CC[C@@H]6c6ccccc6)cc5)CC4)C3)CCN21)C1CCC(=O)NC1=O] | (4aS)-N-(2,6-dioxopiperidin-3-yl)-3-((7- (4-((1R,2S)-6-hydroxy-2-phenyl-1,2,3,4- tetrahydronaphthalen-1-yl)phenyl)-7- azaspiro[3.5]nonan-2-yl)methyl)-N- methyl-1,2,3,4,4a,5- hexahydrobenzo[b]pyrazino[1,2- d][1,4]oxazine-8-carboxamide |
| B313 | 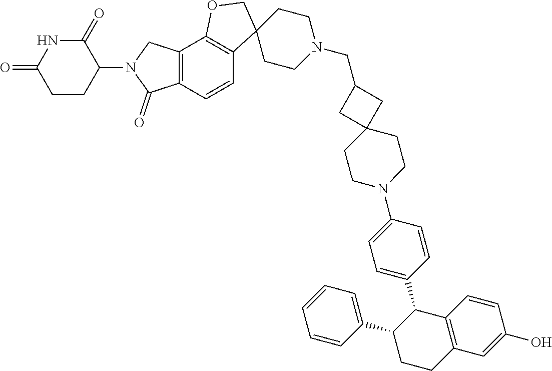 [SMILES: O=C1CCC(N2Cc3c(ccc4c3OCC43CCN(CC4CC5(CCN(c6ccc([C@@H]7c8ccc(O)cc8CC[C@@H]7c7ccccc7)cc6)CC5)C4)CC3)C2=O)C(=O)N1] | 3-(1′-((7-(4-((1R,2S)-6-hydroxy-2-phenyl- 1,2,3,4-tetrahydronaphthalen-1-yl)phenyl)- 7-azaspiro[3.5]nonan-2-yl)methyl)-6-oxo- 6,8-dihydro-2H,7H-spiro[furo[2,3- e]isoindole-3,4′-piperidin]-7-yl)piperidine- 2,6-dione |
| B314 | 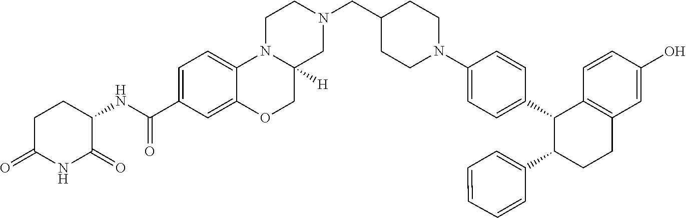 [SMILES: O=C1CC[C@H](NC(=O)c2ccc3c(c2)OC[C@@H]2CN(CC4CCN(c5ccc([C@@H]6c7ccc(O)cc7CC[C@@H]6c6ccccc6)cc5)CC4)CCN32)C(=O)N1] | (S)-N-((S)-2,6-dioxopiperidin-3-yl)-3-((1- (4-((1R,2S)-6-hydroxy-2-phenyl-1,2,3,4- tetrahydronaphthalen-1- yl)phenyl)piperidin-4-yl)methyl)- 1,2,3,4,4a,5- hexahydrobenzo[b]pyrazino[1,2- d][1,4]oxazine-8-carboxamide |
| B315 | 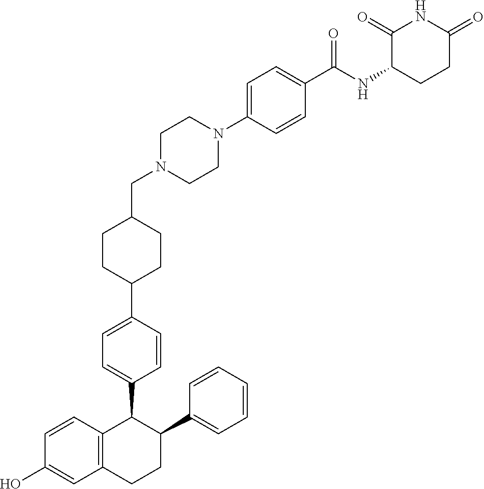 [SMILES: O=C1CC[C@H](NC(=O)c2ccc(N3CCN(CC4CCC(c5ccc([C@@H]6c7ccc(O)cc7CC[C@@H]6c6ccccc6)cc5)CC4)CC3)cc2)C(=O)N1] | N-((S)-2,6-dioxopiperidin-3-yl)-4-(4-((1- (4-((1R,2S)-6-hydroxy-2-phenyl-1,2,3,4- tetrahydronaphthalen-1- yl)phenyl)piperidin-4-yl)methyl)piperazin- 1-yl)benzamide |
| B316 | 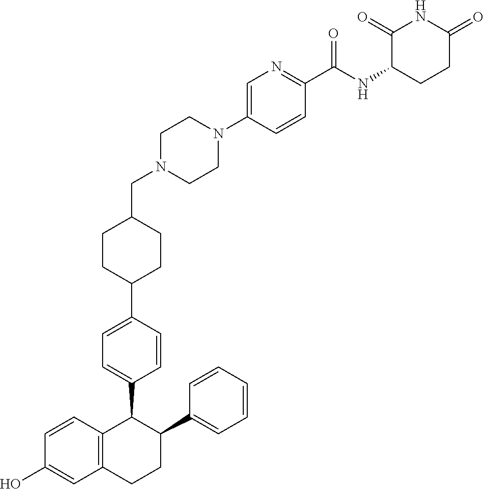 [SMILES: O=C1CC[C@H](NC(=O)c2ccc(N3CCN(CC4CCC(c5ccc([C@@H]6c7ccc(O)cc7CC[C@@H]6c6ccccc6)cc5)CC4)CC3)cn2)C(=O)N1] | N-((S)-2,6-dioxopiperidin-3-yl)-5-(4-((1- (4-((1R,2S)-6-hydroxy-2-phenyl-1,2,3,4- tetrahydronaphthalen-1- yl)phenyl)piperidin-4-yl)methyl)piperazin- 1-yl)picolinamide |
| B317 | 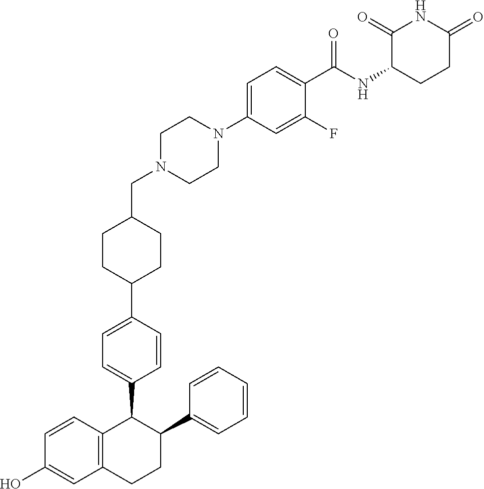 [SMILES: O=C1CC[C@H](NC(=O)c2ccc(N3CCN(CC4CCC(c5ccc([C@@H]6c7ccc(O)cc7CC[C@@H]6c6ccccc6)cc5)CC4)CC3)cc2F)C(=O)N1] | N-((S)-2,6-dioxopiperidin-3-yl)-2-fluoro- 4-(4-((1-(4-((1R,2S)-6-hydroxy-2-phenyl- 1,2,3,4-tetrahydronaphthalen-1- yl)phenyl)piperidin-4-yl)methyl)piperazin- 1-yl)benzamide |
| B318 | 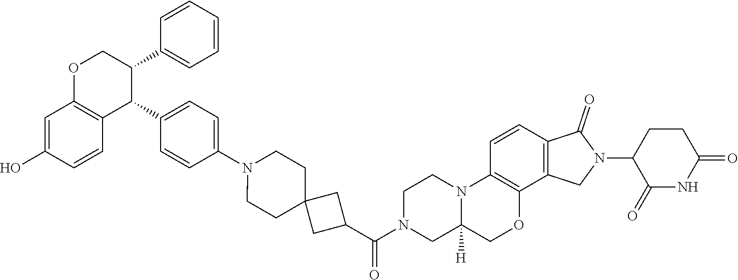 [SMILES: O=C1CCC(N2Cc3c(ccc4c3OC[C@H]3CN(C(=O)C5CC6(CCN(c7ccc([C@@H]8c9ccc(O)cc9OC[C@@H]8c8ccccc8)cc7)CC6)C5)CCN43)C2=O)C(=O)N1] | 3-((R)-7-(7-(4-((1R,2S)-6-hydroxy-2- phenyl-1,2,3,4-tetrahydronaphthalen-1- yl)phenyl)-7-azaspiro[3.5]nonane-2- carbonyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B319 | 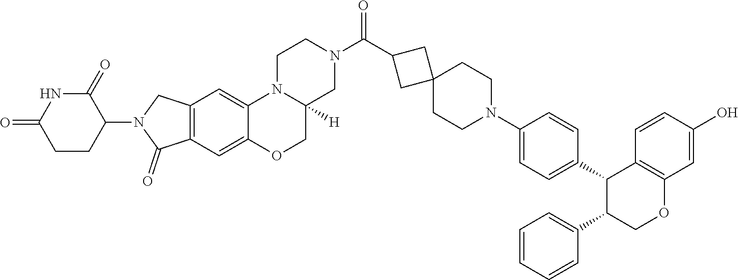 [SMILES: O=C1CCC(N2Cc3cc4c(cc3C2=O)OC[C@@H]2CN(C(=O)C3CC5(CCN(c6ccc([C@@H]7c8ccc(O)cc8OC[C@@H]7c7ccccc7)cc6)CC5)C3)CCN42)C(=O)N1] | 3-((S)-3-(7-(4-((1R,2S)-6-hydroxy-2- phenyl-1,2,3,4-tetrahydronaphthalen-1- yl)phenyl)-7-azaspiro[3.5]nonane-2- carbonyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B320 | 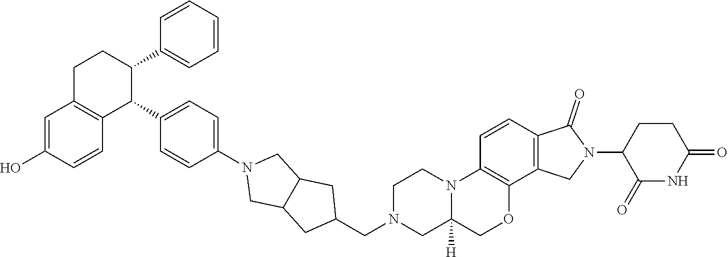 [SMILES: O=C1CCC(N2Cc3c(ccc4c3OC[C@H]3CN(CC5CC6CN(c7ccc([C@@H]8c9ccc(O)cc9CC[C@@H]8c8ccccc8)cc7)CC6C5)CCN43)C2=O)C(=O)N1] | 3-((5aR)-7-(((3aRS,6aSR)-2-(4-((1R,2S)- 6-hydroxy-2-phenyl-1,2,3,4- tetrahydronaphthalen-1- yl)phenyl)octahydrocyclopenta[c]pyrrol-5- yl)methyl)-1-oxo-1,3,5,5a,6,7,8,9- octahydro-2H- pyrazino[1′,2′:4,5][1,4]oxazino[2,3- c]isoindol-2-yl)piperidine-2,6-dione |
| B321 | 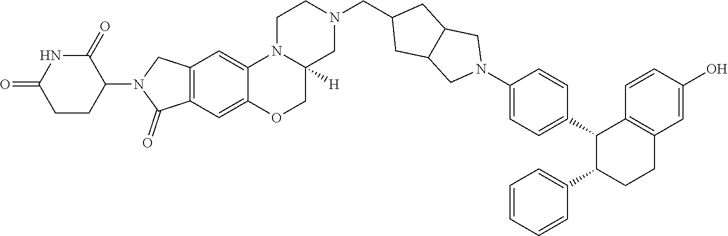 [SMILES: O=C1CCC(N2Cc3cc4c(cc3C2=O)OC[C@@H]2CN(CC3CC5CN(c6ccc([C@@H]7c8ccc(O)cc8CC[C@@H]7c7ccccc7)cc6)CC5C3)CCN42)C(=O)N1] | 3-((4aS)-3-(((3aRS,6aSR)-2-(4-((1R,2S)- 6-hydroxy-2-phenyl-1,2,3,4- tetrahydronaphthalen-1- yl)phenyl)octahydrocyclopenta[c]pyrrol-5- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B322 | 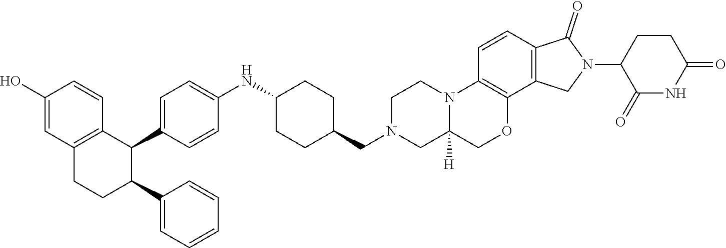 [SMILES: O=C1CCC(N2Cc3c(ccc4c3OC[C@H]3CN(C[C@H]5CC[C@H](Nc6ccc([C@@H]7c8ccc(O)cc8CC[C@@H]7c7ccccc7)cc6)CC5)CCN43)C2=O)C(=O)N1] | 3-((R)-7-(((1R,4R)-4-((4-((1R,2S)-6- hydroxy-2-phenyl-1,2,3,4- tetrahydronaphthalen-1- yl)phenyl)amino)cyclohexyl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| B326 | 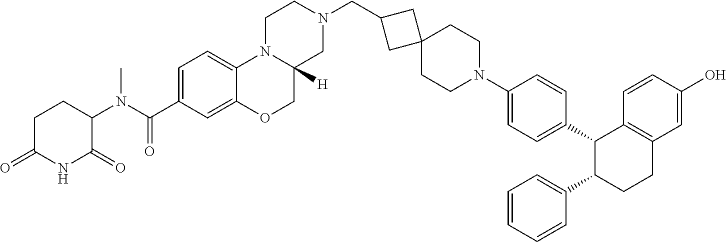 [SMILES: CN(C(=O)c1ccc2c(c1)OC[C@H]1CN(CC3CC4(CCN(c5ccc([C@@H]6c7ccc(O)cc7CC[C@@H]6c6ccccc6)cc5)CC4)C3)CCN21)C1CCC(=O)NC1=O] | (4aR)-N-(2,6-dioxopiperidin-3-yl)-3-((7- (4-((1R,2S)-6-hydroxy-2-phenyl-1,2,3,4- tetrahydronaphthalen-1-yl)phenyl)-7- azaspiro[3.5]nonan-2-yl)methyl)-N- methyl-1,2,3,4,4a,5- hexahydrobenzo[b]pyrazino[1,2- d][1,4]oxazine-8-carboxamide |
| B327 | 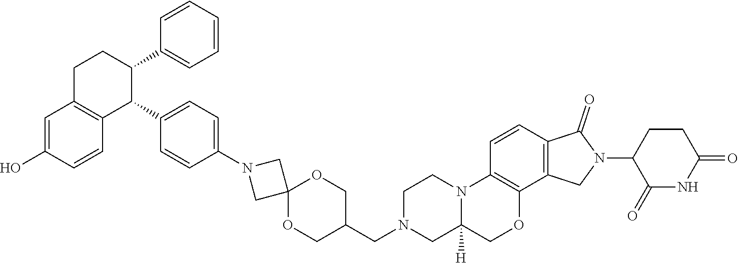 [SMILES: O=C1CCC(N2Cc3c(ccc4c3OC[C@H]3CN(CC5COC6(CN(c7ccc([C@@H]8c9ccc(O)cc9CC[C@@H]8c8ccccc8)cc7)C6)OC5)CCN43)C2=O)C(=O)N1] | 3-((R)-7-((2-(4-((1R,2S)-6-hydroxy-2- phenyl-1,2,3,4-tetrahydronaphthalen-1- yl)phenyl)-5,9-dioxa-2- azaspiro[3.5]nonan-7-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B328 | 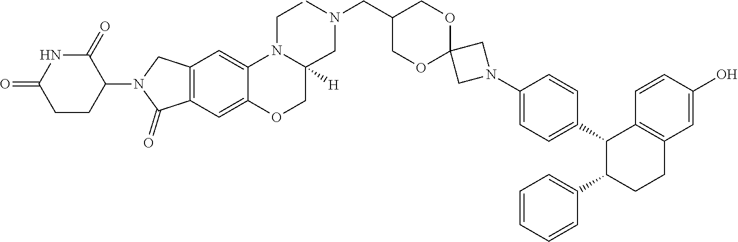 [SMILES: CCN1c2cc3c(cc2OC[C@@H]1CN(C)CC1COC2(CN(c4ccc([C@@H]5c6ccc(O)cc6CC[C@@H]5c5ccccc5)cc4)C2)OC1)C(=O)N(C1CCC(=O)NC1=O)C3] | 3-((S)-3-((2-(4-((1R,2S)-6-hydroxy-2- phenyl-1,2,3,4-tetrahydronaphthalen-1- yl)phenyl)-5,9-dioxa-2- azaspiro[3.5]nonan-7-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B329 | 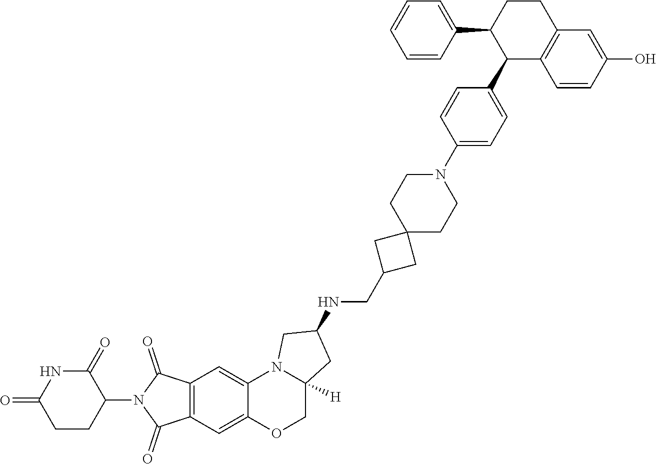 [SMILES: O=C1CCC(N2C(=O)c3cc4c(cc3C2=O)N2C[C@@H](NCC3CC5(CCN(c6ccc([C@@H]7c8ccc(O)cc8CC[C@@H]7c7ccccc7)cc6)CC5)C3)C[C@H]2CO4)C(=O)N1] | (2S,3aS)-8-(2,6-dioxopiperidin-3-yl)-2- (((7-(4-((1R,2S)-6-hydroxy-2-phenyl- 1,2,3,4-tetrahydronaphthalen-1-yl)phenyl)- 7-azaspiro[3.5]nonan-2-yl)methyl)amino)- 2,3,3a,4-tetrahydro-1H,7H- pyrrolo[1′,2′:4,5][1,4]oxazino[2,3- f]isoindole-7,9(8H)-dione |
| B330 | 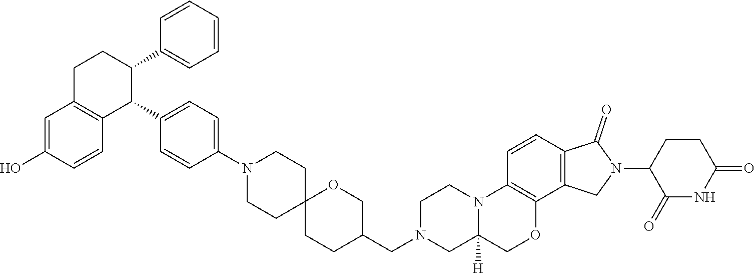 [SMILES: O=C1CCC(N2Cc3c(ccc4c3OC[C@H]3CN(CC5CCC6(CCN(c7ccc([C@@H]8c9ccc(O)cc9CC[C@@H]8c8ccccc8)cc7)CC6)OC5)CCN43)C2=O)C(=O)N1] | 3-((5aR)-7-((9-(4-((1R,2S)-6-hydroxy-2- phenyl-1,2,3,4-tetrahydronaphthalen-1- yl)phenyl)-1-oxa-9-azaspiro[5.5]undecan- 3-yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B330X | 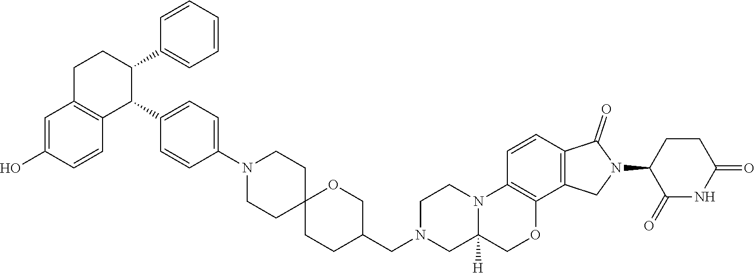 [SMILES: O=C1CC[C@H](N2Cc3c(ccc4c3OC[C@H]3CN(CC5CCC6(CCN(c7ccc([C@@H]8c9ccc(O)cc9CC[C@@H]8c8ccccc8)cc7)CC6)OC5)CCN43)C2=O)C(=O)N1] | (3S)-3-((5aR)-7-((9-(4-((1R,2S)-6- hydroxy-2-phenyl-1,2,3,4- tetrahydronaphthalen-1-yl)phenyl)-1-oxa- 9-azaspiro[5.5]undecan-3-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| B331 | 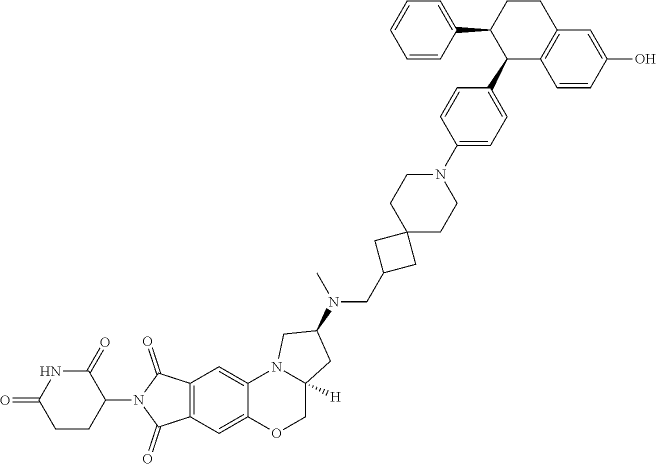 [SMILES: CN(CC1CC2(CCN(c3ccc([C@@H]4c5ccc(O)cc5CC[C@@H]4c4ccccc4)cc3)CC2)C1)[C@H]1C[C@H]2COc3cc4c(cc3N2C1)C(=O)N(C1CCC(=O)NC1=O)C4=O] | (2S,3aS)-8-(2,6-dioxopiperidin-3-yl)-2- (((7-(4-((1R,2S)-6-hydroxy-2-phenyl- 1,2,3,4-tetrahydronaphthalen-1-yl)phenyl)- 7-azaspiro[3.5]nonan-2- yl)methyl)(methyl)amino)-2,3,3a,4- tetrahydro-1H,7H- pyrrolo[1′,2′:4,5][1,4]oxazino[2,3- f]isoindole-7,9(8H)-dione |
| B332 | 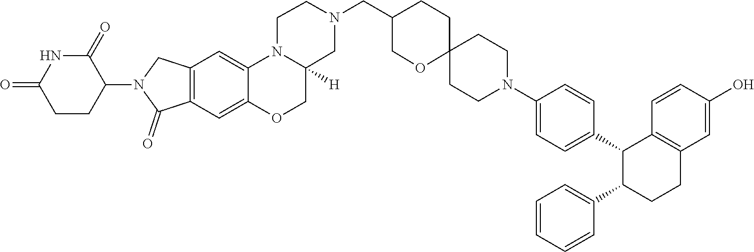 [SMILES: O=C1CCC(N2Cc3cc4c(cc3C2=O)OC[C@@H]2CN(CC3CCC5(CCN(c6ccc([C@@H]7c8ccc(O)cc8CC[C@@H]7c7ccccc7)cc6)CC5)OC3)CCN42)C(=O)N1] | 3-((4aS)-3-((9-(4-((1R,2S)-6-hydroxy-2- phenyl-1,2,3,4-tetrahydronaphthalen-1- yl)phenyl)-1-oxa-9-azaspiro[5.5]undecan- 3-yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B333 | 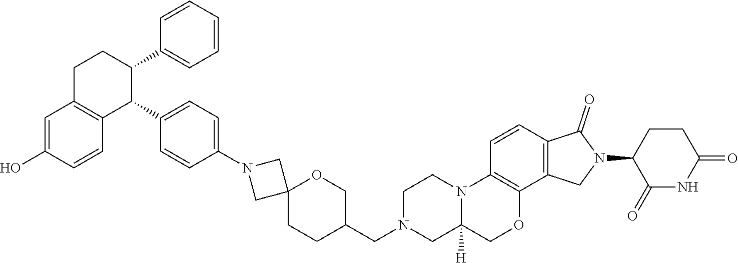 [SMILES: O=C1CC[C@H](N2Cc3c(ccc4c3OC[C@H]3CN(CC5CCC6(CN(c7ccc([C@@H]8c9ccc(O)cc9CC[C@@H]8c8ccccc8)cc7)C6)OC5)CCN43)C2=O)C(=O)N1] | (3S)-3-((5aR)-7-((2-(4-((1R,2S)-6- hydroxy-2-phenyl-1,2,3,4- tetrahydronaphthalen-1-yl)phenyl)-5-oxa- 2-azaspiro[3.5]nonan-7-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B334 | 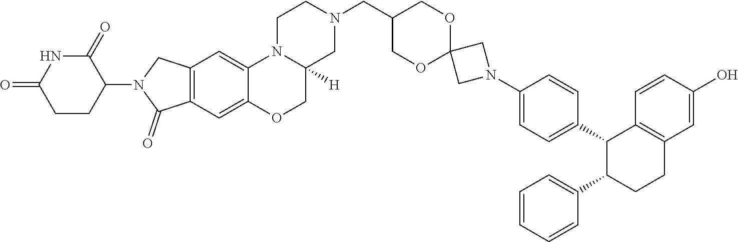 [SMILES: O=C1CCC(N2Cc3cc4c(cc3C2=O)OC[C@@H]2CN(CC3COC5(CN(c6ccc([C@@H]7c8ccc(O)cc8CC[C@@H]7c7ccccc7)cc6)C5)OC3)CCN42)C(=O)N1] | 3-((4aS)-3-((2-(4-((1R,2S)-6-hydroxy-2- phenyl-1,2,3,4-tetrahydronaphthalen-1- yl)phenyl)-5-oxa-2-azaspiro[3.5]nonan-7- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B335 | 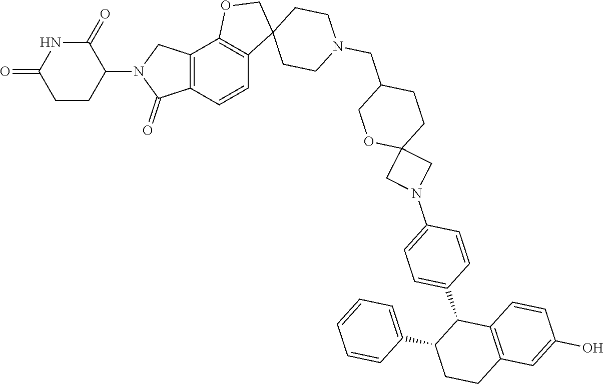 [SMILES: O=C1CCC(N2Cc3c(ccc4c3OCC43CCN(CC4CCC5(CN(c6ccc([C@@H]7c8ccc(O)cc8CC[C@@H]7c7ccccc7)cc6)C5)OC4)CC3)C2=O)C(=O)N1] | 3-(1′-((2-(4-((1R,2S)-6-hydroxy-2-phenyl- 1,2,3,4-tetrahydronaphthalen-1-yl)phenyl)- 5-oxa-2-azaspiro[3.5]nonan-7-yl)methyl)- 6-oxo-6,8-dihydro-2H,7H-spiro[furo[2,3- e]isoindole-3,4′-piperidin]-7-yl)piperidine- 2,6-dione |
| B336 | 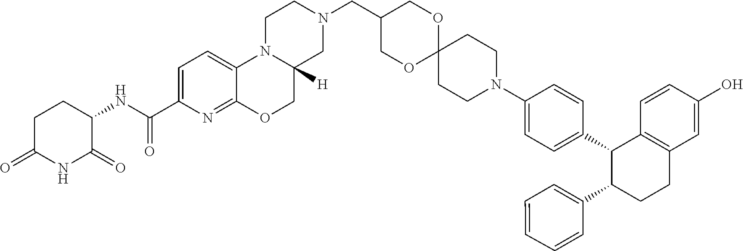 [SMILES: O=C1CC[C@H](NC(=O)c2ccc3c(n2)OC[C@H]2CN(CC4COC5(CCN(c6ccc([C@@H]7c8ccc(O)cc8CC[C@@H]7c7ccccc7)cc6)CC5)OC4)CCN32)C(=O)N1] | (R)-N-((S)-2,6-dioxopiperidin-3-yl)-3-((9- (4-((1R,2S)-6-hydroxy-2-phenyl-1,2,3,4- tetrahydronaphthalen-1-yl)phenyl)-1,5- dioxa-9-azaspiro[5.5]undecan-3- yl)methyl)-1,2,3,4,4a,5- hexahydropyrazino[1,2-d]pyrido[2,3- b][1,4]oxazine-8-carboxamide |
| B337 | 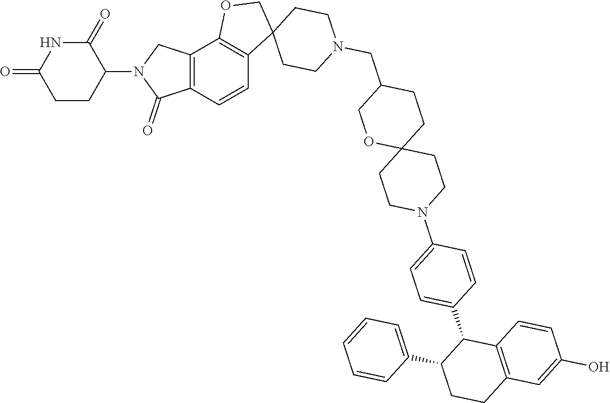 [SMILES: O=C1CCC(N2Cc3c(ccc4c3OCC43CCN(CC4CCC5(CCN(c6ccc([C@@H]7c8ccc(O)cc8CC[C@@H]7c7ccccc7)cc6)CC5)OC4)CC3)C2=O)C(=O)N1] | 3-(1′-((9-(4-((1R,2S)-6-hydroxy-2-phenyl- 1,2,3,4-tetrahydronaphthalen-1-yl)phenyl)- 1,5-dioxa-9-azaspiro[5.5]undecan-3- yl)methyl)-6-oxo-6,8-dihydro-2H,7H- spiro[furo[2,3-e]isoindole-3,4′-piperidin]- 7-yl)piperidine-2,6-dione |
| B337X | 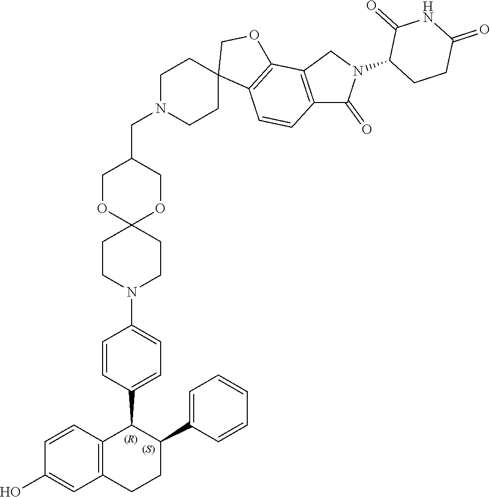 | (S)-3-(1′-((9-(4-((1R,2S)-6-hydroxy-2- phenyl-1,2,3,4-tetrahydronaphthalen-1- yl)phenyl)-1,5-dioxa-9- azaspiro[5.5]undecan-3-yl)methyl)-6-oxo- 6,8-dihydro-2H,7H-spiro[furo[2,3- e]isoindole-3,4′-piperidin]-7-yl)piperidine- 2,6-dione |
| B339 | 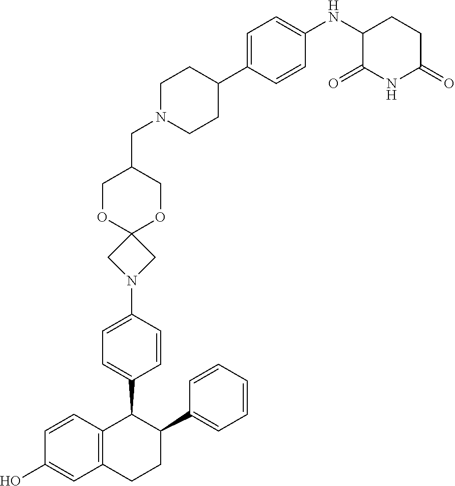 [SMILES: O=C1CCC(Nc2ccc(C3CCN(CC4COC5(CN(c6ccc([C@@H]7c8ccc(O)cc8CC[C@@H]7c7ccccc7)cc6)C5)OC4)CC3)cc2)C(=O)N1] | 3-((4-(1-((2-(4-((1R,2S)-6-hydroxy-2- phenyl-1,2,3,4-tetrahydronaphthalen-1- yl)phenyl)-5,9-dioxa-2- azaspiro[3.5]nonan-7-yl)methyl)piperidin- 4-yl)phenyl)amino)piperidine-2,6-dione |
| B340 | 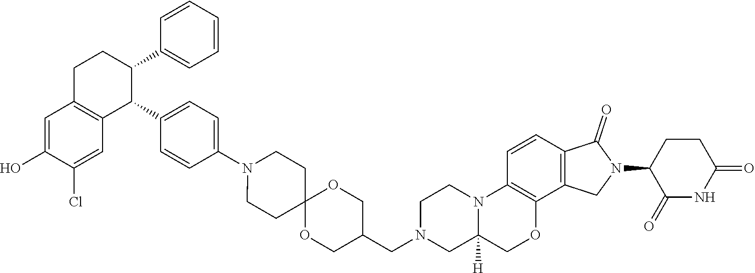 [SMILES: O=C1CC[C@H](N2Cc3c(ccc4c3OC[C@H]3CN(CC5COC6(CCN(c7ccc([C@@H]8c9cc(Cl)c(O)cc9CC[C@@H]8c8ccccc8)cc7)CC6)OC5)CCN43)C2=O)C(=O)N1] | (S)-3-((R)-7-((9-(4-((1R,2S)-7-chloro-6- hydroxy-2-phenyl-1,2,3,4- tetrahydronaphthalen-1-yl)phenyl)-1,5- dioxa-9-azaspiro[5.5]undecan-3- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B341 | 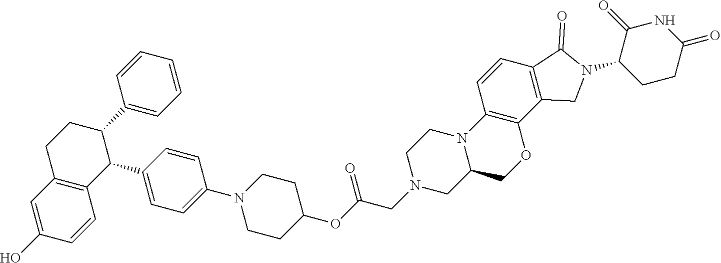 [SMILES: O=C1CC[C@H](N2Cc3c(ccc4c3OC[C@H]3CN(CC(=O)OC5CCN(c6ccc([C@@H]7c8ccc(O)cc8CC[C@@H]7c7ccccc7)cc6)CC5)CCN43)C2=O)C(=O)N1] | 1-(4-((1R,2S)-6-hydroxy-2-phenyl-1,2,3,4- tetrahydronaphthalen-1- yl)phenyl)piperidin-4-yl 2-((R)-2-((S)-2,6- dioxopiperidin-3-yl)-1-oxo-2,3,5a,6,8,9- hexahydro-1H- pyrazino[1′,2′:4,5][1,4]oxazino[2,3- elisoindol-7(5H)-yl)acetate |
| B342 | 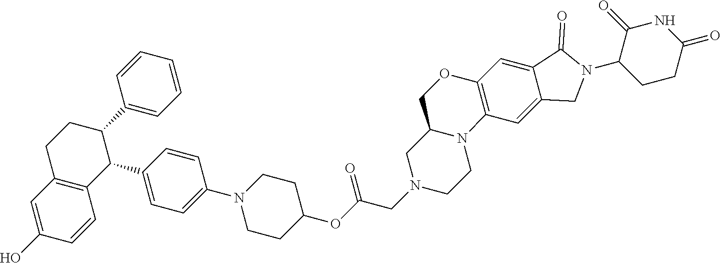 [SMILES: O=C1CCC(N2Cc3cc4c(cc3C2=O)OC[C@@H]2CN(CC(=O)OC3CCN(c5ccc([C@@H]6c7ccc(O)cc7CC[C@@H]6c6ccccc6)cc5)CC3)CCN42)C(=O)N1] | 1-(4-((1R,2S)-6-hydroxy-2-phenyl-1,2,3,4- tetrahydronaphthalen-1- yl)phenyl)piperidin-4-yl 2-((4aS)-9-(2,6- dioxopiperidin-3-yl)-8-oxo-1,2,4a,5,9,10- hexahydro-8H- pyrazino[1′,2′:4,5][1,4]oxazino[2,3- f]isoindol-3(4H)-yl)acetate |
| B343 | 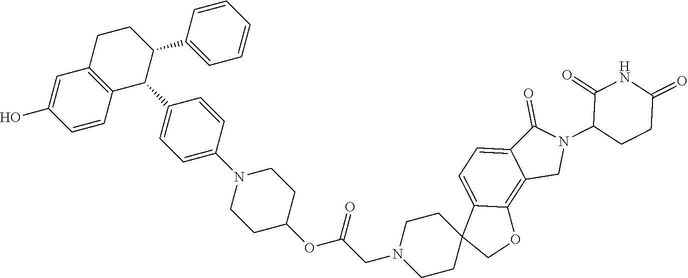 [SMILES: O=C1CCC(N2Cc3c(ccc4c3OCC43CCN(CC(=O)OC4CCN(c5ccc([C@@H]6c7ccc(O)cc7CC[C@@H]6c6ccccc6)cc5)CC4)CC3)C2=O)C(=O)N1] | 1-(4-((1R,2S)-6-hydroxy-2-phenyl-1,2,3,4- tetrahydronaphthalen-1- yl)phenyl)piperidin-4-yl 2-(7-((S)-2,6- dioxopiperidin-3-yl)-6-oxo-7,8-dihydro- 2H,6H-spiro[furo[2,3-c]isoindolc-3,4′- piperidin]-1′-yl)acetate |
| B344 | 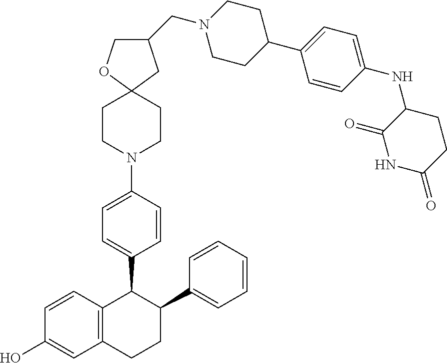 [SMILES: O=C1CCC(Nc2ccc(C3CCN(CC4COC5(CCN(c6ccc([C@@H]7c8ccc(O)cc8CC[C@@H]7c7ccccc7)cc6)CC5)C4)CC3)cc2)C(=O)N1] | 3-((4-(1-((8-(4-((1R,2S)-6-hydroxy-2- phenyl-1,2,3,4-tetrahydronaphthalen-1- yl)phenyl)-1-oxa-8-azaspiro[4.5]decan-3- yl)methyl)piperidin-4- yl)phenyl)amino)piperidine-2,6-dione |
| B345 | 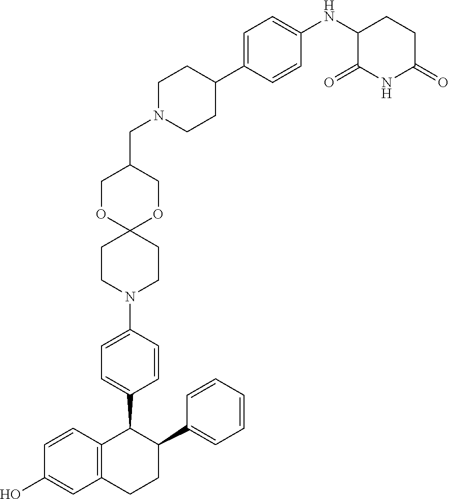 [SMILES: O=C1CCC(Nc2ccc(C3CCN(CC4COC5(CCN(c6ccc([C@@H]7c8ccc(O)cc8CC[C@@H]7c7ccccc7)cc6)CC5)OC4)CC3)cc2)C(=O)N1] | 3-((4-(1-((9-(4-((1R,2S)-6-hydroxy-2- phenyl-1,2,3,4-tetrahydronaphthalen-1- yl)phenyl)-1,5-dioxa-9- azaspiro[5.5]undecan-3- yl)methyl)piperidin-4- yl)phenyl)amino)piperidine-2,6-dione |
| B346 | 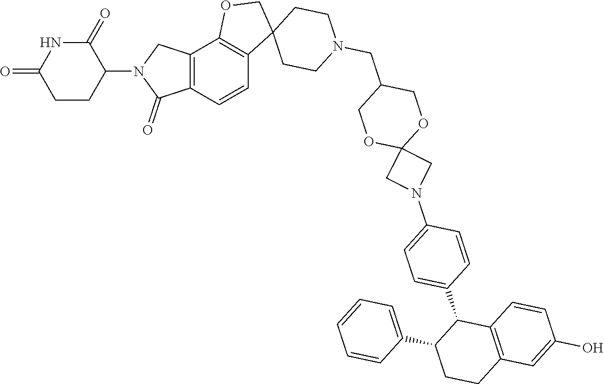 [SMILES: O=C1CCC(N2Cc3c(ccc4c3OCC43CCN(CC4COC5(CN(c6ccc([C@@H]7c8ccc(O)cc8CC[C@@H]7c7ccccc7)cc6)C5)OC4)CC3)C2=O)C(=O)N1] | 3-(1′-((2-(4-((1R,2S)-6-hydroxy-2-phenyl- 1,2,3,4-tetrahydronaphthalen-1-yl)phenyl)- 5,9-dioxa-2-azaspiro[3.5]nonan-7- yl)methyl)-6-oxo-6,8-dihydro-2H,7H- spiro[furo[2,3-e]isoindole-3,4′-piperidin]- 7-yl)piperidine-2,6-dione |
| B347 | 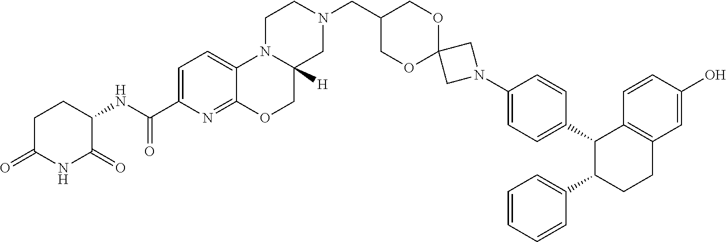 [SMILES: O=C1CC[C@H](NC(=O)c2ccc3c(n2)OC[C@H]2CN(CC4COC5(CN(c6ccc([C@@H]7c8ccc(O)cc8CC[C@@H]7c7ccccc7)cc6)C5)OC4)CCN32)C(=O)N1] | (R)-N-((S)-2,6-dioxopiperidin-3-yl)-3-((2- (4-((1R,2S)-6-hydroxy-2-phenyl-1,2,3,4- tetrahydronaphthalen-1-yl)phenyl)-5,9- dioxa-2-azaspiro[3.5]nonan-7-yl)methyl)- 1,2,3,4,4a,5-hexahydropyrazino[1,2- d]pyrido[2,3-b][1,4]oxazine-8- carboxamide |
| B348 | 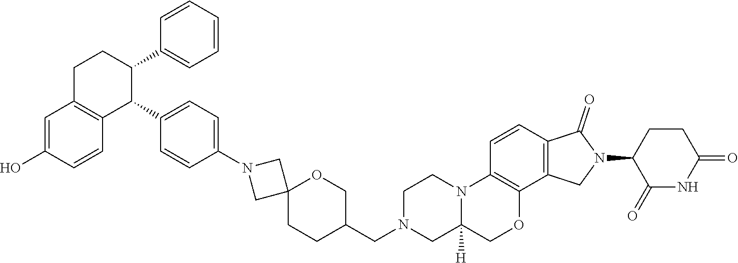 [SMILES: O=C1CC[C@H](N2Cc3c(ccc4c3OC[C@H]3CN(CC5CCC6(CN(c7ccc([C@@H]8c9ccc(O)cc9CC[C@@H]8c8ccccc8)cc7)C6)OC5)CCN43)C2=O)C(=O)N1] | (3S)-3-((5aR)-7-((2-(4-((1R,2S)-6- hydroxy-2-phenyl-1,2,3,4- tetrahydronaphthalen-1-yl)phenyl)-5-oxa- 2-azaspiro[3.5]nonan-7-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B349 | 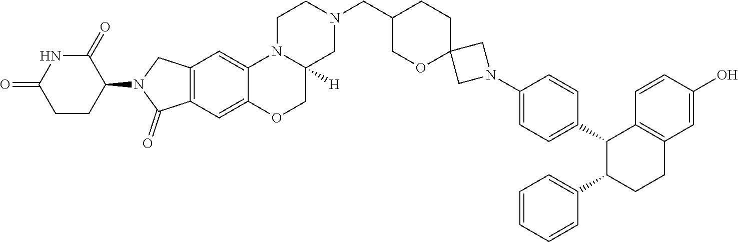 [SMILES: O=C1CC[C@H](N2Cc3cc4c(cc3C2=O)OC[C@@H]2CN(CC3CCC5(CN(c6ccc([C@@H]7c8ccc(O)cc8CC[C@@H]7c7ccccc7)cc6)C5)OC3)CCN42)C(=O)N1] | (3S)-3-((4aS)-3-((2-(4-((1R,2S)-6- hydroxy-2-phenyl-1,2,3,4- tetrahydronaphthalen-1-yl)phenyl)-5-oxa- 2-azaspiro[3.5]nonan-7-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B350 | 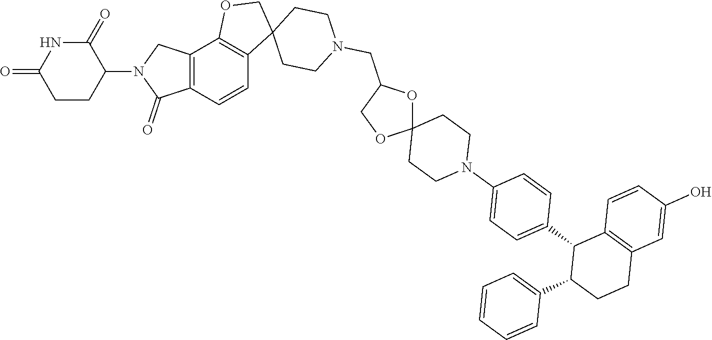 [SMILES: O=C1CCC(N2Cc3c(ccc4c3OCC43CCN(CC4COC5(CCN(c6ccc([C@@H]7c8ccc(O)cc8CC[C@@H]7c7ccccc7)cc6)CC5)O4)CC3)C2=O)C(=O)N1] | 3-(1′-((8-(4-((1R,2S)-6-hydroxy-2-phenyl- 1,2,3,4-tetrahydronaphthalen-1-yl)phenyl)- 1,4-dioxa-8-azaspiro[4.5]decan-2- yl)methyl)-6-oxo-6,8-dihydro-2H,7H- spiro[furo[2,3-e]isoindole-3,4′-piperidin]- 7-yl)piperidine-2,6-dione |
| B351 | 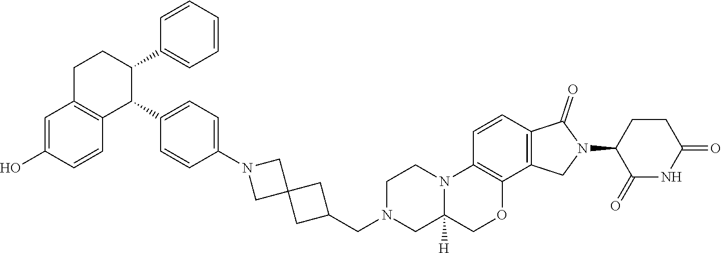 [SMILES: O=C1CC[C@H](N2Cc3c(ccc4c3OC[C@H]3CN(CC5CC6(C5)CN(c5ccc([C@@H]7c8ccc(O)cc8CC[C@@H]7c7ccccc7)cc5)C6)CCN43)C2=O)C(=O)N1] | (S)-3-((R)-7-((2-(4-((1R,2S)-6-hydroxy-2- phenyl-1,2,3,4-tetrahydronaphthalen-1- yl)phenyl)-2-azaspiro[3.3]heptan-6- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| B353 | 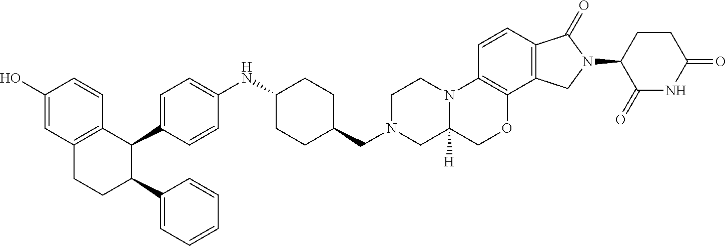 [SMILES: O=C1CC[C@H](N2Cc3c(ccc4c3OC[C@H]3CN(C[C@H]5CC[C@H](Nc6ccc([C@@H]7c8ccc(O)cc8CC[C@@H]7c7ccccc7)cc6)CC5)CCN43)C2=O)C(=O)N1] | (S)-3-((R)-7-(((1R,4R)-4-((4-((1R,2S)-6- hydroxy-2-phenyl-1,2,3,4- tetrahydronaphthalen-1- yl)phenyl)amino)cyclohexyl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| B354 | 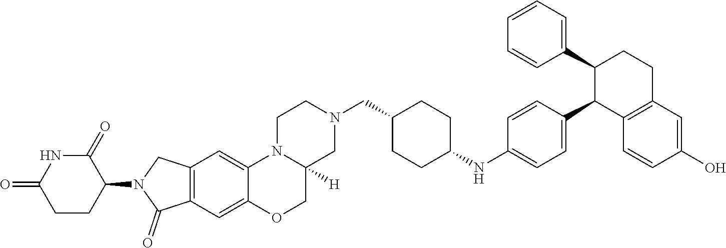 [SMILES: O=C1CC[C@H](N2Cc3cc4c(cc3C2=O)OC[C@@H]2CN(C[C@H]3CC[C@@H](Nc5ccc([C@@H]6c7ccc(O)cc7CC[C@@H]6c6ccccc6)cc5)CC3)CCN42)C(=O)N1] | (S)-3-((S)-3-(((1R,4S)-4-((4-((1R,2S)-6- hydroxy-2-phenyl-1,2,3,4- tetrahydronaphthalen-1- yl)phenyl)amino)cyclohexyl)methyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| B357 | 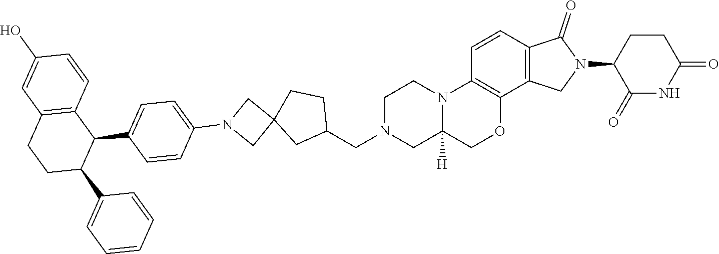 [SMILES: O=C1CC[C@H](N2Cc3c(ccc4c3OC[C@H]3CN(CC5CCC6(C5)CN(c5ccc([C@@H]7c8ccc(O)cc8CC[C@@H]7c7ccccc7)cc5)C6)CCN43)C2=O)C(=O)N1] | (3S)-3-((5aR)-7-((2-(4-((1R,2S)-6- hydroxy-2-phenyl-1,2,3,4- tetrahydronaphthalen-1-yl)phenyl)-2- azaspiro[3.4]octan-6-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B358 | 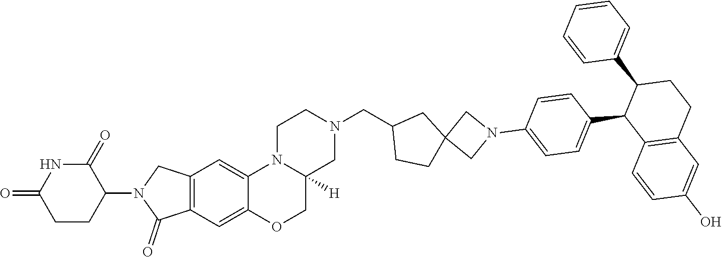 [SMILES: O=C1CCC(N2Cc3cc4c(cc3C2=O)OC[C@@H]2CN(CC3CCC5(C3)CN(c3ccc([C@@H]6c7ccc(O)cc7CC[C@@H]6c6ccccc6)cc3)C5)CCN42)C(=O)N1] | 3-((4aS)-3-((2-(4-((1R,2S)-6-hydroxy-2- phenyl-1,2,3,4-tetrahydronaphthalen-1- yl)phenyl)-2-azaspiro[3.4]octan-6- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B359 | 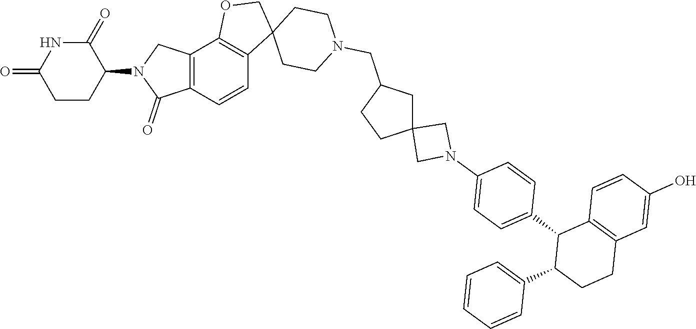 [SMILES: O=C1CC[C@H](N2Cc3c(ccc4c3OCC43CCN(CC4CCC5(C4)CN(c4ccc([C@@H]6c7ccc(O)cc7CC[C@@H]6c6ccccc6)cc4)C5)CC3)C2=O)C(=O)N1] | 3-(1′-((2-(4-((1R,2S)-6-hydroxy-2-phenyl- 1,2,3,4-tetrahydronaphthalen-1-yl)phenyl)- 2-azaspiro[3.4]octan-6-yl)methyl)-6-oxo- 6,8-dihydro-2H,7H-spiro[furo[2,3- e]isoindole-3,4′-piperidin]-7-yl)piperidine- 2,6-dione |
| B360 | 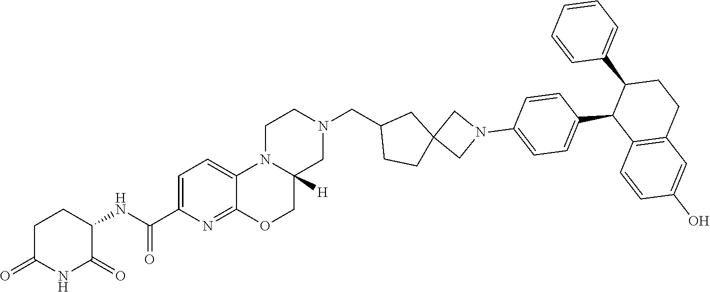 [SMILES: O=C1CC[C@H](NC(=O)c2ccc3c(n2)OC[C@H]2CN(CC4CCC5(C4)CN(c4ccc([C@@H]6c7ccc(O)cc7CC[C@@H]6c6ccccc6)cc4)C5)CCN32)C(=O)N1] | (4aR)-N-((S)-2,6-dioxopiperidin-3-yl)-3- ((2-(4-((1R,2S)-6-hydroxy-2-phenyl- 1,2,3,4-tetrahydronaphthalen-1-yl)phenyl)- 2-azaspiro[3.4]octan-6-yl)methyl)- 1,2,3,4,4a,5-hexahydropyrazino[1,2- d]pyrido[2,3-b][1,4]oxazine-8- carboxamide |
| B362 | 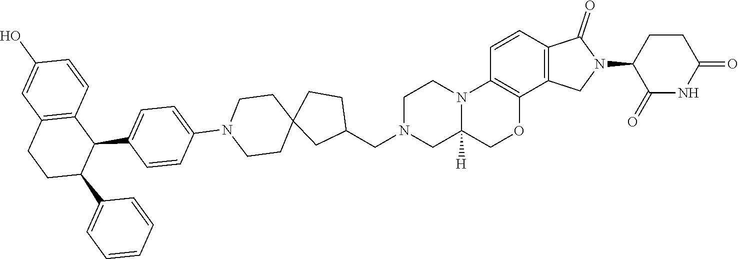 [SMILES: O=C1CC[C@H](N2Cc3c(ccc4c3OC[C@H]3CN(CC5CCC6(CCN(c7ccc([C@@H]8c9ccc(O)cc9CC[C@@H]8c8ccccc8)cc7)CC6)C5)CCN43)C2=O)C(=O)N1] | (3S)-3-((5aR)-7-((8-(4-((1R,2S)-6- hydroxy-2-phenyl-1,2,3,4- tetrahydronaphthalen-1-yl)phenyl)-8- azaspiro[4.5]decan-2-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B363 | 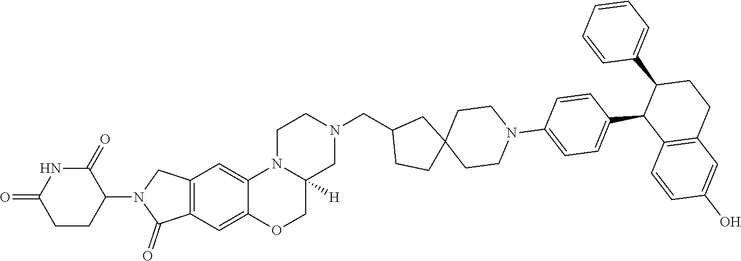 [SMILES: O=C1CCC(N2Cc3cc4c(cc3C2=O)OC[C@@H]2CN(CC3CCC5(CCN(c6ccc([C@@H]7c8ccc(O)cc8CC[C@@H]7c7ccccc7)cc6)CC5)C3)CCN42)C(=O)N1] | 3-((4aS)-3-((8-(4-((1R,2S)-6-hydroxy-2- phenyl-1,2,3,4-tetrahydronaphthalen-1- yl)phenyl)-8-azaspiro[4.5]decan-2- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| B364 | 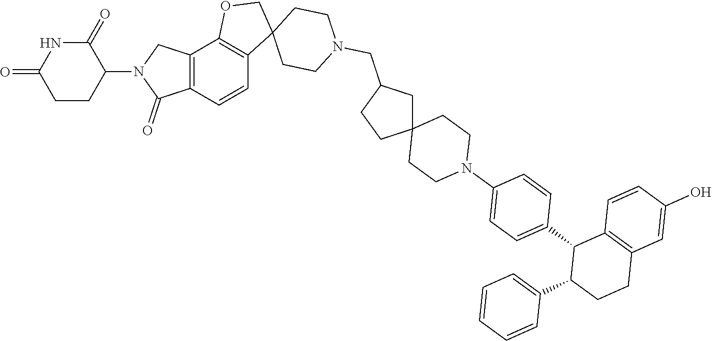 [SMILES: O=C1CCC(N2Cc3c(ccc4c3OCC43CCN(CC4CCC5(CCN(c6ccc([C@@H]7c8ccc(O)cc8CC[C@@H]7c7ccccc7)cc6)CC5)C4)CC3)C2=O)C(=O)N1] | 3-(1′-((8-(4-((1R,2S)-6-hydroxy-2-phenyl- 1,2,3,4-tetrahydronaphthalen-1-yl)phenyl)- 8-azaspiro[4.5]decan-2-yl)methyl)-6-oxo- 6,8-dihydro-2H,7H-spiro[furo[2,3- e]isoindole-3,4′-piperidin]-7-yl)piperidine- 2,6-dione |
| B365 | 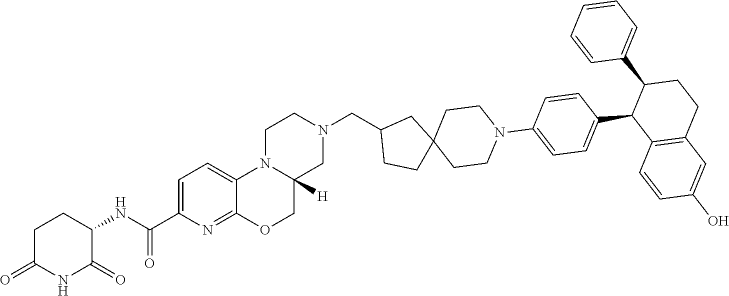 [SMILES: O=C1CC[C@H](NC(=O)c2ccc3c(n2)OC[C@H]2CN(CC4CCC5(CCN(c6ccc([C@@H]7c8ccc(O)cc8CC[C@@H]7c7ccccc7)cc6)CC5)C4)CCN32)C(=O)N1] | (4aR)-N-((S)-2,6-dioxopiperidin-3-yl)-3- ((8-(4-((1R,2S)-6-hydroxy-2-phenyl- 1,2,3,4-tetrahydronaphthalen-1-yl)phenyl)- 8-azaspiro[4.5]decan-2-yl)methyl)- 1,2,3,4,4a,5-hexahydropyrazino[1,2- d]pyrido[2,3-b][1,4]oxazine-8- carboxamide |
| B366 | 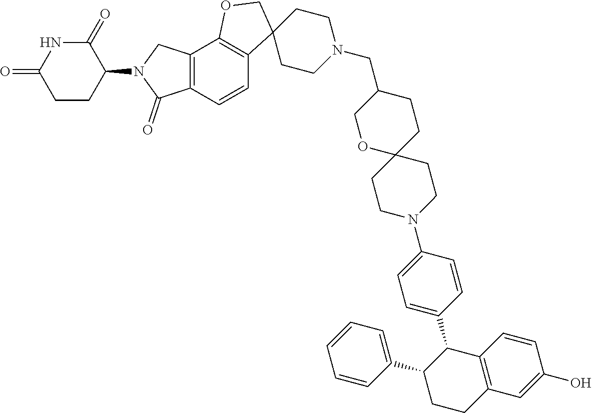 [SMILES: O=C1CC[C@H](N2Cc3c(ccc4c3OCC43CCN(CC4CCC5(CCN(c6ccc([C@@H]7c8ccc(O)cc8CC[C@@H]7c7ccccc7)cc6)CC5)OC4)CC3)C2=O)C(=O)N1] | (3S)-3-(1′-((9-(4-((1R,2S)-6-hydroxy-2- phenyl-1,2,3,4-tetrahydronaphthalen-1- yl)phenyl)-1-oxa-9-azaspiro[5.5]undecan- 3-yl)methyl)-6-oxo-6,8-dihydro-2H,7H- spiro[furo[2,3-e]isoindole-3,4′-piperidin]- 7-yl)piperidine-2,6-dione |
| B366X | 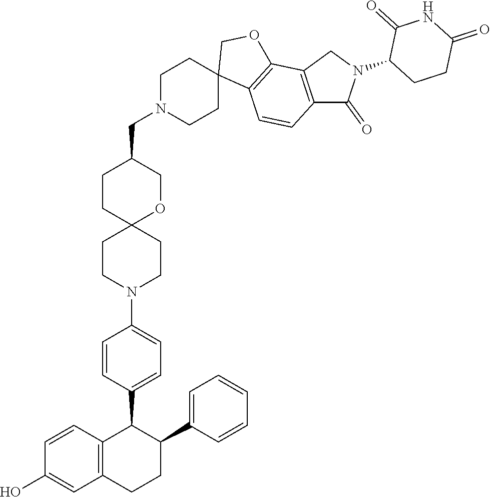 [SMILES: O=C1CC[C@H](N2Cc3c(ccc4c3OCC43CCN(C[C@@H]4CCC5(CCN(c6ccc([C@@H]7c8ccc(O)cc8CC[C@@H]7c7ccccc7)cc6)CC5)OC4)CC3)C2=O)C(=O)N1] | (S)-3-(1′-(((S)-9-(4-((1R,2S)-6-hydroxy-2- phenyl-1,2,3,4-tetrahydronaphthalen-1- yl)phenyl)-1-oxa-9-azaspiro[5.5]undecan- 3-yl)methyl)-6-oxo-6,8-dihydro-2H,7H- spiro[furo[2,3-e]isoindole-3,4′-piperidin]- 7-yl)piperidine-2,6-dione |
| B366Y | 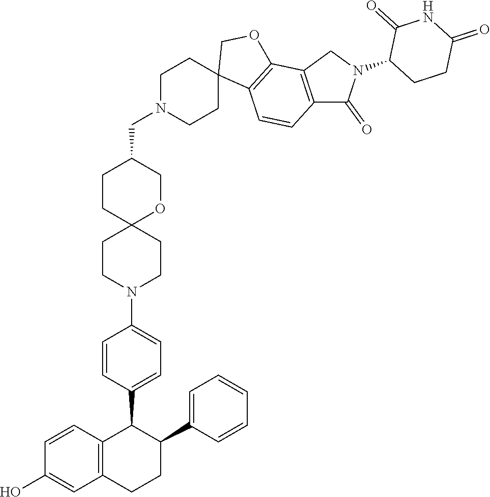 [SMILES: O=C1CC[C@H](N2Cc3c(ccc4c3OCC43CCN(C[C@H]4CCC5(CCN(c6ccc([C@@H]7c8ccc(O)cc8CC[C@@H]7c7ccccc7)cc6)CC5)OC4)CC3)C2=O)C(=O)N1] | (S)-3-(1′-(((R)-9-(4-((1R,2S)-6-hydroxy-2- phenyl-1,2,3,4-tetrahydronaphthalen-1- yl)phenyl)-1-oxa-9-azaspiro[5.5]undecan- 3-yl)methyl)-6-oxo-6,8-dihydro-2H,7H- spiro[furo[2,3-e]isoindole-3,4′-piperidin]- 7-yl)piperidine-2,6-dione |
| B367 | 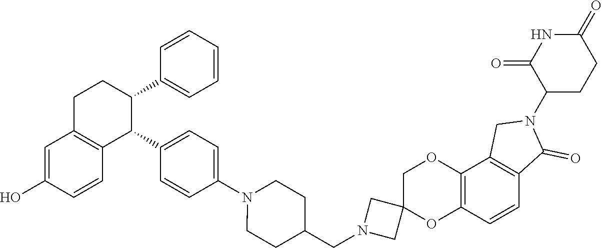 [SMILES: O=C1CCC(N2Cc3c(ccc4c3OCC3(CN(CC5CCN(c6ccc([C@@H]7c8ccc(O)cc8CC[C@@H]7c7ccccc7)cc6)CC5)C3)O4)C2=O)C(=O)N1] | 3-(1-((1-(4-((1R,2S)-6-hydroxy-2-phenyl- 1,2,3,4-tetrahydronaphthalen-1- yl)phenyl)piperidin-4-yl)methyl)-7′-oxo- 7′,9′-dihydro-2′H,8′H-spiro[azetidine-3,3′- [1,4]dioxino[2,3-e]isoindol]-8′- yl)piperidine-2,6-dione |
| B368 | 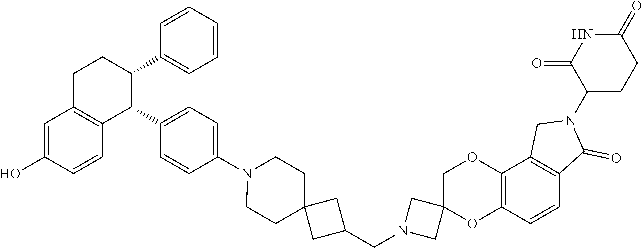 [SMILES: O=C1CCC(N2Cc3c(ccc4c3OCC3(CN(CC5CC6(CCN(c7ccc([C@@H]8c9ccc(O)cc9CC[C@@H]8c8ccccc8)cc7)CC6)C5)C3)O4)C2=O)C(=O)N1] | 3-(1-((7-(4-((1R,2S)-6-hydroxy-2-phenyl- 1,2,3,4-tetrahydronaphthalen-1-yl)phenyl)- 7-azaspiro[3.5]nonan-2-yl)methyl)-7′-oxo- 7′,9′-dihydro-2′H,8′H-spiro[azetidine-3,3′- [1,4]dioxino[2,3-e]isoindol]-8′- yl)piperidine-2,6-dione |
| B369 | 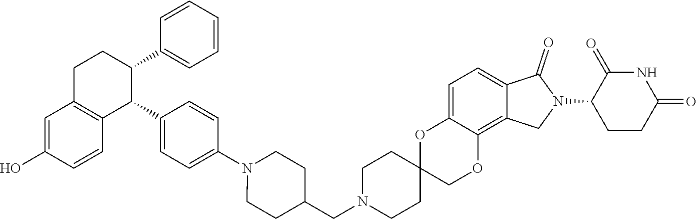 [SMILES: O=C1CC[C@H](N2Cc3c(ccc4c3OCC3(CCN(CC5CCN(c6ccc([C@@H]7c8ccc(O)cc8CC[C@@H]7c7ccccc7)cc6)CC5)CC3)O4)C2=O)C(=O)N1] | (S)-3-(1-((1-(4-((1R,2S)-6-hydroxy-2- phenyl-1,2,3,4-tetrahydronaphthalen-1- yl)phenyl)piperidin-4-yl)methyl)-7′-oxo- 7′,9′-dihydro-2′H,8′H-spiro[piperidine-4,3′- [1,4]dioxino[2,3-e]isoindol]-8′- yl)piperidine-2,6-dione |
| B370 | 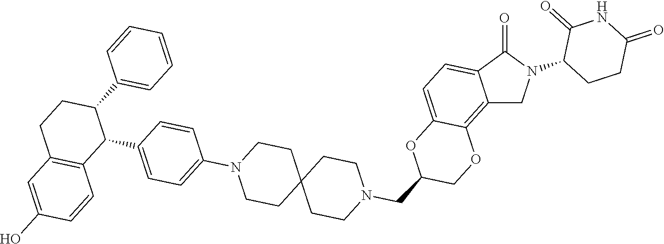 [SMILES: O=C1CC[C@H](N2Cc3c(ccc4c3OC[C@@H](CN3CCC5(CC3)CCN(c3ccc([C@@H]6c7ccc(O)cc7CC[C@@H]6c6ccccc6)cc3)CC5)O4)C2=O)C(=O)N1] | (S)-3-((R)-3-((9-(4-((1R,2S)-6-hydroxy-2- phenyl-1,2,3,4-tetrahydronaphthalen-1- yl)phenyl)-3,9-diazaspiro[5.5]undecan-3- yl)methyl)-7-oxo-2,3,7,9-tetrahydro-8H- [1,4]dioxino[2,3-e]isoindol-8- yl)piperidine-2,6-dione |
| B371 | 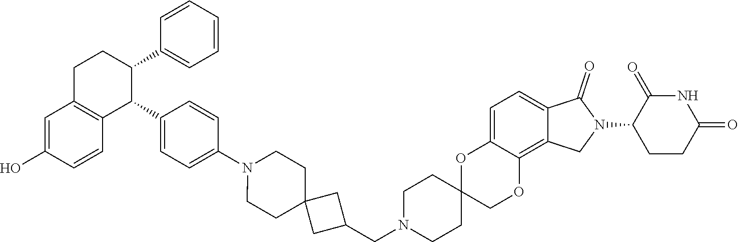 [SMILES: O=C1CC[C@H](N2Cc3c(ccc4c3OCC3(CCN(CC5CC6(CCN(c7ccc([C@@H]8c9ccc(O)cc9CC[C@@H]8c8ccccc8)cc7)CC6)C5)CC3)O4)C2=O)C(=O)N1] | (S)-3-(1-((7-(4-((1R,2S)-6-hydroxy-2- phenyl-1,2,3,4-tetrahydronaphthalen-1- yl)phenyl)-7-azaspiro[3.5]nonan-2- yl)methyl)-7′-oxo-7′,9′-dihydro-2′H,8′H- spiro[piperidine-4,3′-[1,4]dioxino[2,3- e]isoindol]-8′-yl)piperidine-2,6-dione |
| B372 | 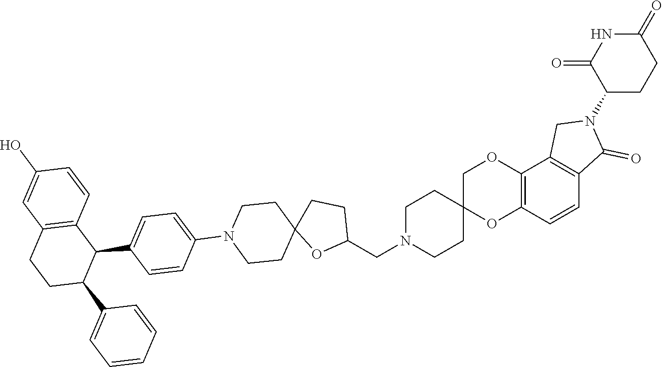 [SMILES: O=C1CC[C@H](N2Cc3c(ccc4c3OCC3(CCN(CC5CCC6(CCN(c7ccc([C@@H]8c9ccc(O)cc9CC[C@@H]8c8ccccc8)cc7)CC6)O5)CC3)O4)C2=O)C(=O)N1] | (3S)-3-(1-((8-(4-((1R,2S)-6-hydroxy-2- phenyl-1,2,3,4-tetrahydronaphthalen-1- yl)phenyl)-1-oxa-8-azaspiro[4.5]decan-2- yl)methyl)-7′-oxo-7′,9′-dihydro-2′H,8′H- spiro[piperidine-4,3′-[1,4]dioxino[2,3- e]isoindol]-8′-yl)piperidine-2,6-dione |
| B373 | 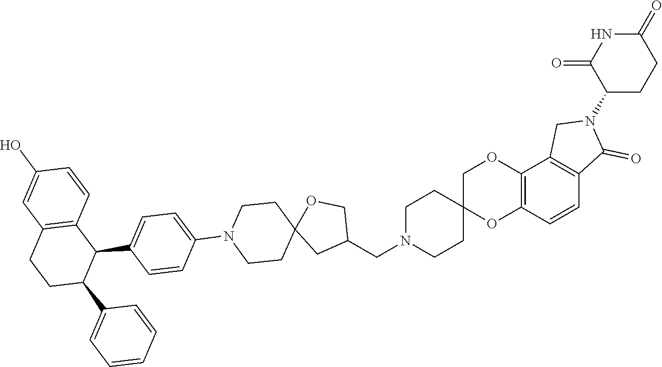 [SMILES: O=C1CC[C@H](N2Cc3c(ccc4c3OCC3(CCN(CC5COC6(CCN(c7ccc([C@@H]8c9ccc(O)cc9CC[C@@H]8c8ccccc8)cc7)CC6)C5)CC3)O4)C2=O)C(=O)N1] | (3S)-3-(1-((8-(4-((1R,2S)-6-hydroxy-2- phenyl-1,2,3,4-tetrahydronaphthalen-1- yl)phenyl)-1-oxa-8-azaspiro[4.5]decan-3- yl)methyl)-7′-oxo-7′,9′-dihydro-2′H,8′H- spiro[piperidine-4,3′-[1,4]dioxino[2,3- e]isoindol]-8′-yl)piperidine-2,6-dione |
| B374 | 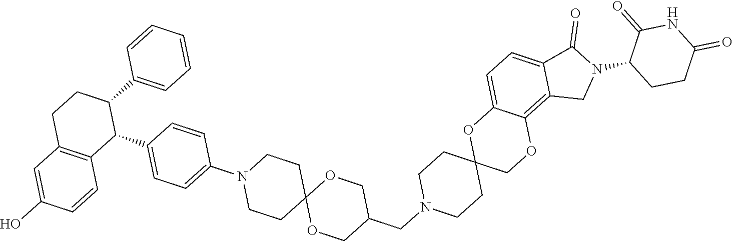 [SMILES: O=C1CC[C@H](N2Cc3c(ccc4c3OCC3(CCN(CC5COC6(CCN(c7ccc([C@@H]8c9ccc(O)cc9CC[C@@H]8c8ccccc8)cc7)CC6)OC5)CC3)O4)C2=O)C(=O)N1] | (S)-3-(1-((9-(4-((1R,2S)-6-hydroxy-2- phenyl-1,2,3,4-tetrahydronaphthalen-1- yl)phenyl)-1,5-dioxa-9- azaspiro[5.5]undecan-3-yl)methyl)-7′-oxo- 7′,9′-dihydro-2′H,8′H-spiro[piperidine-4,3′- [1,4]dioxino[2,3-e]isoindol]-8′- yl)piperidine-2,6-dione |
| B375 | 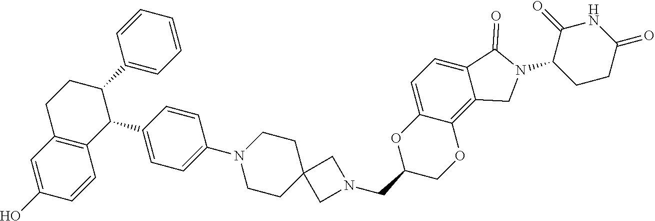 [SMILES: O=C1CC[C@H](N2Cc3c(ccc4c3OC[C@@H](CN3CC5(CCN(c6ccc([C@@H]7c8ccc(O)cc8CC[C@@H]7c7ccccc7)cc6)CC5)C3)O4)C2=O)C(=O)N1] | (S)-3-((R)-3-((7-(4-((1R,2S)-6-hydroxy-2- phenyl-1,2,3,4-tctrahydronaphthalen-1- yl)phenyl)-2,7-diazaspiro[3.5]nonan-2- yl)methyl)-7-oxo-2,3,7,9-tetrahydro-8H- [1,4]dioxino[2,3-e]isoindol-8- yl)piperidine-2,6-dione |
| B376 | 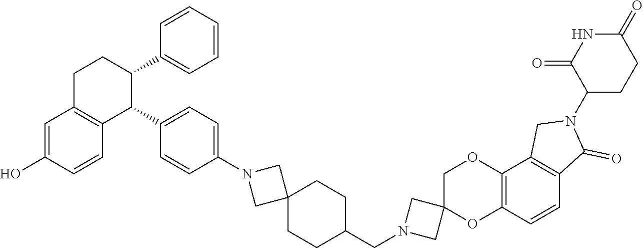 [SMILES: O=C1CCC(N2Cc3c(ccc4c3OCC3(CN(CC5CCC6(CC5)CN(c5ccc([C@@H]7c8ccc(O)cc8CC[C@@H]7c7ccccc7)cc5)C6)C3)O4)C2=O)C(=O)N1] | 3-(1-((2-(4-((1R,2S)-6-hydroxy-2-phenyl- 1,2,3,4-tetrahydronaphthalen-1-yl)phenyl)- 2-azaspiro[3.5]nonan-7-yl)methyl)-7′-oxo- 7′,9′-dihydro-2′H,8′H-spiro[azetidine-3,3′- [1,4]dioxino[2,3-e]isoindol]-8′- yl)piperidine-2,6-dione |
| B377 | 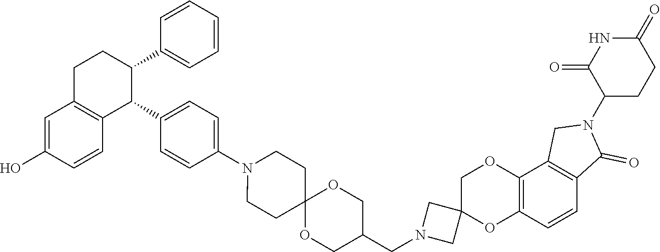 [SMILES: O=C1CCC(N2Cc3c(ccc4c3OCC3(CN(CC5COC6(CCN(c7ccc([C@@H]8c9ccc(O)cc9CC[C@@H]8c8ccccc8)cc7)CC6)OC5)C3)O4)C2=O)C(=O)N1] | 3-(1-((9-(4-((1R,2S)-6-hydroxy-2-phenyl- 1,2,3,4-tetrahydronaphthalen-1-yl)phenyl)- 1,5-dioxa-9-azaspiro[5.5]undecan-3- yl)methyl)-7′-oxo-7′,9′-dihydro-2′H,8′H- spiro[azetidine-3,3′-[1,4]dioxino[2,3- e]isoindol]-8′-yl)piperidine-2,6-dione |
| B378 | 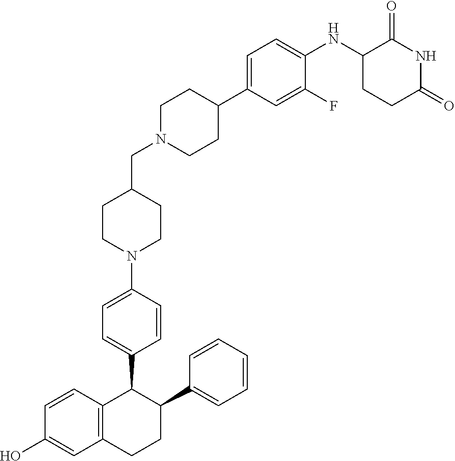 [SMILES: O=C1CCC(Nc2ccc(C3CCN(CC4CCN(c5ccc([C@@H]6c7ccc(O)cc7CC[C@@H]6c6ccccc6)cc5)CC4)CC3)cc2F)C(=O)N1] | 3-((2-fluoro-4-(1-((1-(4-((1R,2S)-6- hydroxy-2-phenyl-1,2,3,4- tetrahydronaphthalen-1- yl)phenyl)piperidin-4-yl)methyl)piperidin- 4-yl)phenyl)amino)piperidine-2,6-dione |
| B379 | 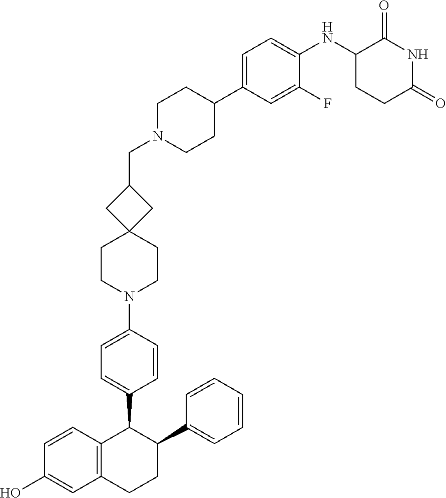 [SMILES: O=C1CCC(Nc2ccc(C3CCN(CC4CC5(CCN(c6ccc([C@@H]7c8ccc(O)cc8CC[C@@H]7c7ccccc7)cc6)CC5)C4)CC3)cc2F)C(=O)N1] | 3-((2-fluoro-4-(1-((7-(4-((1R,2S)-6- hydroxy-2-phenyl-1,2,3,4- tetrahydronaphthalen-1-yl)phenyl)-7- azaspiro[3.5]nonan-2-yl)methyl)piperidin- 4-yl)phenyl)amino)piperidine-2,6-dione |
| B380 | 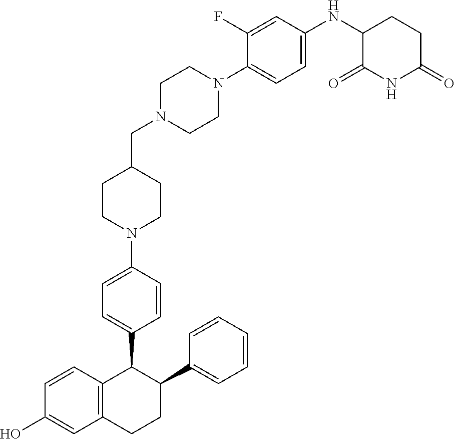 [SMILES: O=C1CCC(Nc2ccc(N3CCN(CC4CCN(c5ccc([C@@H]6c7ccc(O)cc7CC[C@@H]6c6ccccc6)cc5)CC4)CC3)c(F)c2)C(=O)N1] | 3-((3-fluoro-4-(4-((1-(4-((1R,2S)-6- hydroxy-2-phenyl-1,2,3,4- tetrahydronaphthalen-1- yl)phenyl)piperidin-4-yl)methyl)piperazin- 1-yl)phenyl)amino)piperidine-2,6-dione |
| B381 | 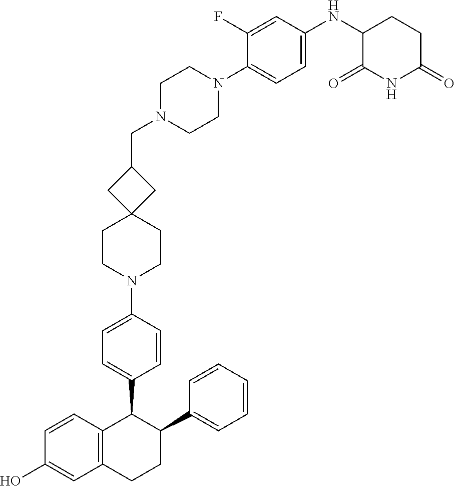 [SMILES: O=C1CCC(Nc2ccc(N3CCN(CC4CC5(CCN(c6ccc([C@@H]7c8ccc(O)cc8CC[C@@H]7c7ccccc7)cc6)CC5)C4)CC3)c(F)c2)C(=O)N1] | 3-((3-fluoro-4-(4-((7-(4-((1R,2S)-6- hydroxy-2-phenyl-1,2,3,4- tetrahydronaphthalen-1-yl)phenyl)-7- azaspiro[3.5]nonan-2-yl)methyl)piperazin- 1-yl)phenyl)amino)piperidine-2,6-dione |
| B382 | 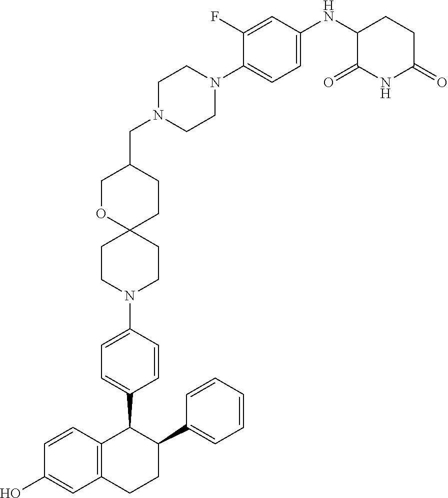 [SMILES: O=C1CCC(Nc2ccc(N3CCN(CC4CCC5(CCN(c6ccc([C@@H]7c8ccc(O)cc8CC[C@@H]7c7ccccc7)cc6)CC5)OC4)CC3)c(F)c2)C(=O)N1] | 3-((3-fluoro-4-(4-((9-(4-((1R,2S)-6- hydroxy-2-phenyl-1,2,3,4- tetrahydronaphthalen-1-yl)phenyl)-1-oxa- 9-azaspiro[5.5]undecan-3- yl)methyl)piperazin-1- yl)phenyl)amino)piperidine-2,6-dione |
| B383 | 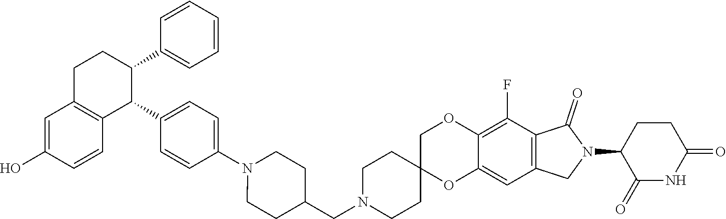 [SMILES: O=C1CC[C@H](N2Cc3cc4c(c(F)c3C2=O)OCC2(CCN(CC3CCN(c5ccc([C@@H]6c7ccc(O)cc7CC[C@@H]6c6ccccc6)cc5)CC3)CC2)O4)C(=O)N1] | (S)-3-(5′-fluoro-1-((1-(4-((1R,2S)-6- hydroxy-2-phenyl-1,2,3,4- tetrahydronaphthalen-1- yl)phenyl)piperidin-4-yl)methyl)-6′-oxo- 6′,8′-dihydro-3′H,7′H-spiro[piperidine-4,2′- [1,4]dioxino[2,3-f]isoindol]-7′- yl)piperidine-2,6-dione |
| B384 | 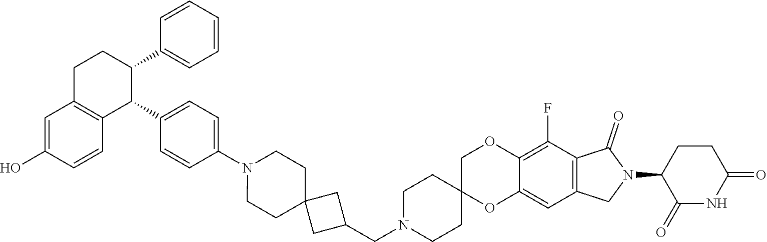 [SMILES: O=C1CC[C@H](N2Cc3cc4c(c(F)c3C2=O)OCC2(CCN(CC3CC5(CCN(c6ccc([C@@H]7c8ccc(O)cc8CC[C@@H]7c7ccccc7)cc6)CC5)C3)CC2)O4)C(=O)N1] | (S)-3-(5′-fluoro-1-((7-(4-((1R,2S)-6- hydroxy-2-phenyl-1,2,3,4- tetrahydronaphthalen-1-yl)phenyl)-7- azaspiro[3.5]nonan-2-yl)methyl)-6′-oxo- 6′,8′-dihydro-3′H,7′H-spiro[piperidine-4,2′- [1,4]dioxino[2,3-f]isoindol]-7′- yl)piperidine-2,6-dione |
| B385 | 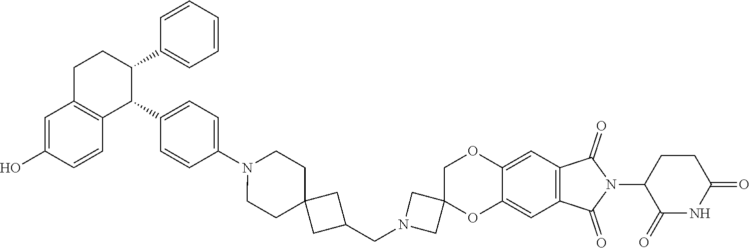 [SMILES: O=C1CCC(N2C(=O)c3cc4c(cc3C2=O)OC2(CO4)CN(CC3CC4(CCN(c5ccc([C@@H]6c7ccc(O)cc7CC[C@@H]6c6ccccc6)cc5)CC4)C3)C2)C(=O)N1] | 7′-(2,6-dioxopiperidin-3-yl)-1-((7-(4- ((1R,2S)-6-hydroxy-2-phenyl-1,2,3,4- tetrahydronaphthalen-1-yl)phenyl)-7- azaspiro[3.5]nonan-2-yl)methyl)-7′-hydro- 3′H,6′H-spiro[azetidine-3,2′- [1,4]dioxino[2,3-f]isoindole]-6′,8′-dione |
| B386 | 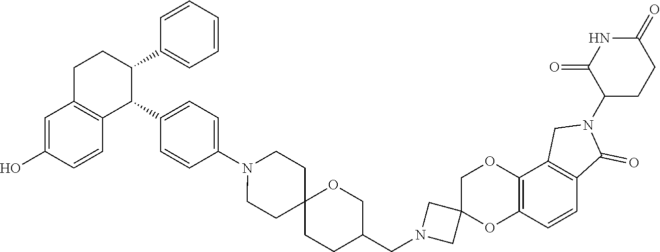 [SMILES: O=C1CCC(N2Cc3c(ccc4c3OCC3(CN(CC5CCC6(CCN(c7ccc([C@@H]8c9ccc(O)cc9CC[C@@H]8c8ccccc8)cc7)CC6)OC5)C3)O4)C2=O)C(=O)N1] | 3-(1-((9-(4-((1R,2S)-6-hydroxy-2-phenyl- 1,2,3,4-tetrahydronaphthalen-1-yl)phenyl)- 1-oxa-9-azaspiro[5.5]undecan-3- yl)methyl)-7′-oxo-7′,9′-dihydro-2′H,8′H- spiro[azetidine-3,3′-[1,4]dioxino[2,3- e]isoindol]-8′-yl)piperidine-2,6-dione |
| B387 | 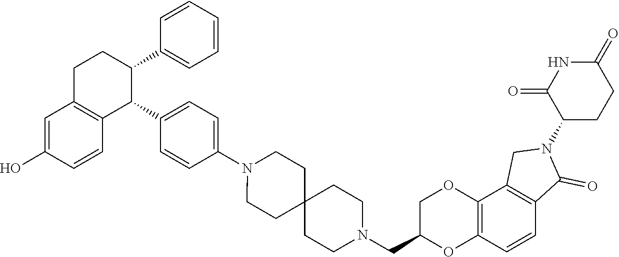 [SMILES: O=C1CC[C@H](N2Cc3c(ccc4c3OC[C@H](CN3CCC5(CC3)CCN(c3ccc([C@@H]6c7ccc(O)cc7CC[C@@H]6c6ccccc6)cc3)CC5)O4)C2=O)C(=O)N1] | (S)-3-((S)-3-((9-(4-((1R,2S)-6-hydroxy-2- phenyl-1,2,3,4-tetrahydronaphthalen-1- yl)phenyl)-3,9-diazaspiro[5.5]undecan-3- yl)methyl)-7-oxo-2,3,7,9-tetrahydro-8H- [1,4]dioxino[2,3-e]isoindol-8- yl)piperidine-2,6-dione |
| B388 | 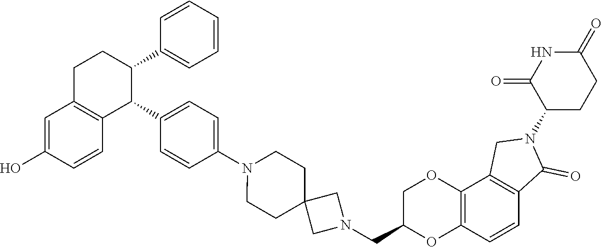 [SMILES: O=C1CC[C@H](N2Cc3c(ccc4c3OC[C@H](CN3CC5(CCN(c6ccc([C@@H]7c8ccc(O)cc8CC[C@@H]7c7ccccc7)cc6)CC5)C3)O4)C2=O)C(=O)N1] | (S)-3-((S)-3-((7-(4-((1R,2S)-6-hydroxy-2- phenyl-1,2,3,4-tetrahydronaphthalen-1- yl)phenyl)-2,7-diazaspiro[3.5]nonan-2- yl)methyl)-7-oxo-2,3,7,9-tetrahydro-8H- [1,4]dioxino[2,3-e]isoindol-8- yl)piperidine-2,6-dione |
| B391 | 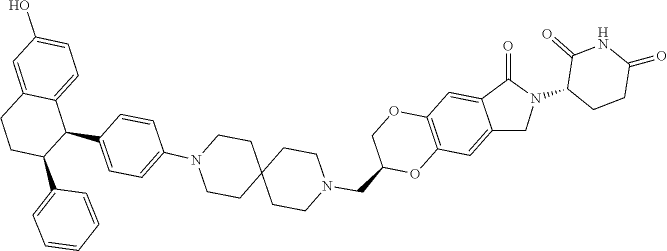 [SMILES: O=C1CC[C@H](N2Cc3cc4c(cc3C2=O)OC[C@H](CN2CCC3(CC2)CCN(c2ccc([C@@H]5c6ccc(O)cc6CC[C@@H]5c5ccccc5)cc2)CC3)O4)C(=O)N1] | (S)-3-((S)-2-((9-(4-((1R,2S)-6-hydroxy-2- phenyl-1,2,3,4-tetrahydronaphthalen-1- yl)phenyl)-3,9-diazaspiro[5.5]undecan-3- yl)methyl)-6-oxo-2,3,6,8-tetrahydro-7H- [1,4]dioxino[2,3-f]isoindol-7- yl)piperidine-2,6-dione |
| B392 | 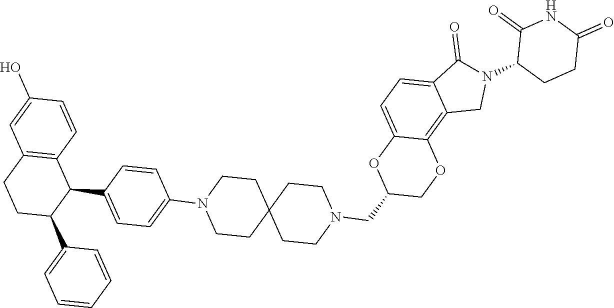 [SMILES: O=C1CC[C@H](N2Cc3c(ccc4c3OC[C@H](CN3CCC5(CC3)CCN(c3ccc([C@@H]6c7ccc(O)cc7CC[C@@H]6c6ccccc6)cc3)CC5)O4)C2=O)C(=O)N1] | (S)-3-((S)-3-((9-(4-((1R,2S)-6-hydroxy-2- phenyl-1,2,3,4-tetrahydronaphthalen-1- yl)phenyl)-3,9-diazaspiro[5.5]undecan-3- yl)methyl)-7-oxo-2,3,7,9-tetrahydro-8H- [1,4]dioxino[2,3-e]isoindol-8- yl)piperidine-2,6-dione |
| B393 | 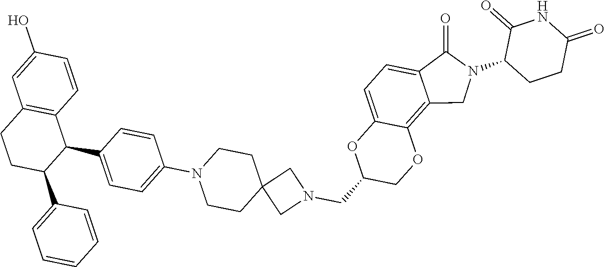 [SMILES: O=C1CC[C@H](N2Cc3c(ccc4c3OC[C@H](CN3CC5(CCN(c6ccc([C@@H]7c8ccc(O)cc8CC[C@@H]7c7ccccc7)cc6)CC5)C3)O4)C2=O)C(=O)N1] | (S)-3-((S)-3-((7-(4-((1R,2S)-6-hydroxy-2- phenyl-1,2,3,4-tetrahydronaphthalen-1- yl)phenyl)-2,7-diazaspiro[3.5]nonan-2- yl)methyl)-7-oxo-2,3,7,9-tetrahydro-8H- [1,4]dioxino[2,3-e]isoindol-8- yl)piperidine-2,6-dione |
| B394 | 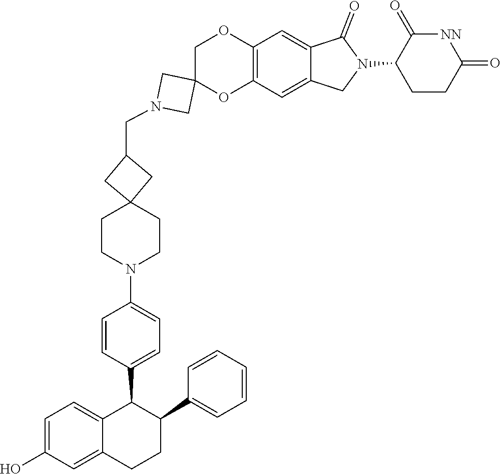 | (S)-3-(1-((7-(4-((1R,2S)-6-hydroxy-2- phenyl-1,2,3,4-tetrahydronaphthalen-1- yl)phenyl)-7-azaspiro[3.5]nonan-2- yl)methyl)-6′-oxo-6′,8′-dihydro-3′H,7′H- spiro[azetidine-3,2′-[1,4]dioxino[2,3- f]isoindol]-7′-yl)piperidine-2,6-dione |
| B395 | 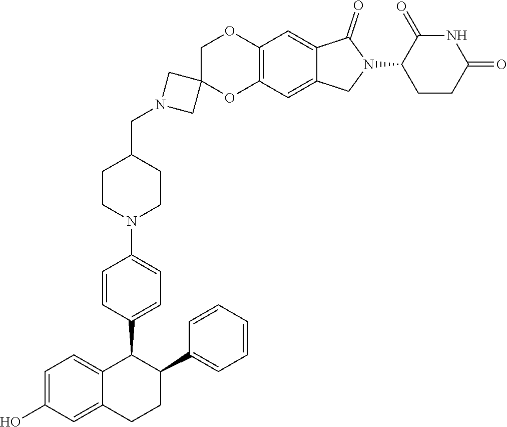 [SMILES: O=C1CC[C@H](N2Cc3cc4c(cc3C2=O)OCC2(CN(CC3CCN(c5ccc([C@@H]6c7ccc(O)cc7CC[C@@H]6c6ccccc6)cc5)CC3)C2)O4)C(=O)N1] | (S)-3-(1-((1-(4-((1R,2S)-6-hydroxy-2- phenyl-1,2,3,4-tetrahydronaphthalen-1- yl)phenyl)piperidin-4-yl)methyl)-6′-oxo- 6′,8′-dihydro-3′H,7′H-spiro[azetidine-3,2′- [1,4]dioxino[2,3-f]isoindol]-7′- yl)piperidine-2,6-dione |
| B396 | 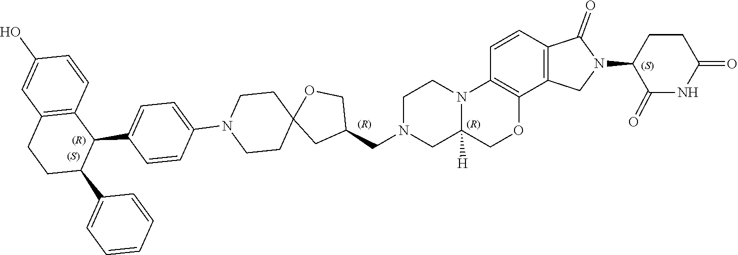 | (S)-3-((R)-7-(((R)-8-(4-((1R,2S)-6- hydroxy-2-phenyl-1,2,3,4- tetrahydronaphthalen-1-yl)phenyl)-1-oxa- 8-azaspiro[4.5]decan-3-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B397 | 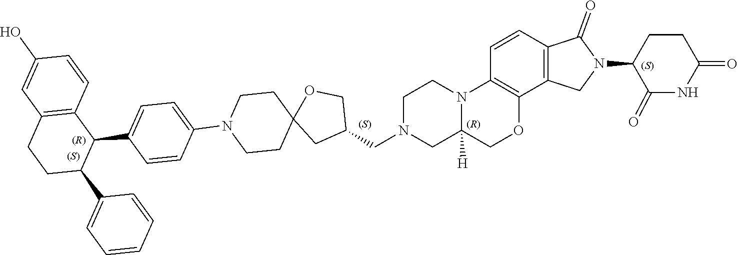 | (S)-3-((R)-7-(((S)-8-(4-((1R,2S)-6- hydroxy-2-phenyl-1,2,3,4- tetrahydronaphthalen-1-yl)phenyl)-1-oxa- 8-azaspiro[4.5]decan-3-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| B398 | 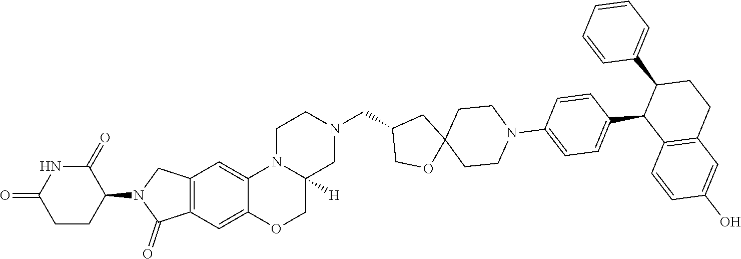 [SMILES: O=C1CC[C@H](N2Cc3cc4c(cc3C2=O)OC[C@@H]2CN(C[C@H]3COC5(CCN(c6ccc([C@@H]7c8ccc(O)cc8CC[C@@H]7c7ccccc7)cc6)CC5)C3)CCN42)C(=O)N1] | (S)-3-((S)-3-(((S)-8-(4-((1R,2S)-6- hydroxy-2-phenyl-1,2,3,4- tetrahydronaphthalen-1-yl)phenyl)-1-oxa- 8-azaspiro[4.5]decan-3-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B399 | 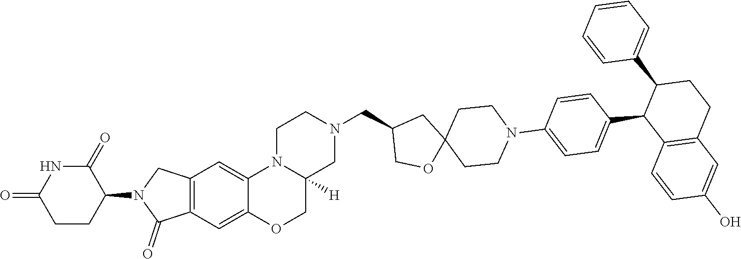 [SMILES: O=C1CC[C@H](N2Cc3cc4c(cc3C2=O)OC[C@@H]2CN(C[C@@H]3COC5(CCN(c6ccc([C@@H]7c8ccc(O)cc8CC[C@@H]7c7ccccc7)cc6)CC5)C3)CCN42)C(=O)N1] | (S)-3-((S)-3-(((R)-8-(4-((1R,2S)-6- hydroxy-2-phenyl-1,2,3,4- tetrahydronaphthalen-1-yl)phenyl)-1-oxa- 8-azaspiro[4.5]decan-3-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| B400 | 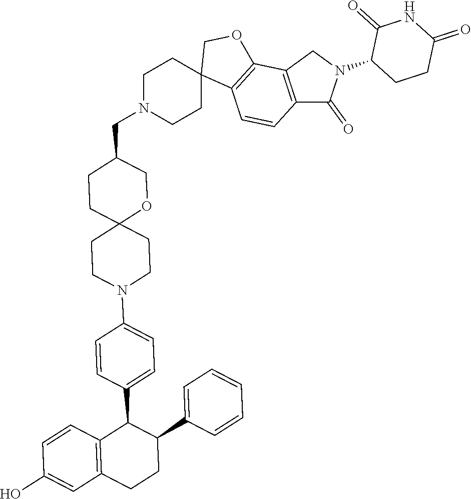 [SMILES: O=C1CC[C@H](N2Cc3c(ccc4c3OCC43CCN(C[C@@H]4CCC5(CCN(c6ccc([C@@H]7c8ccc(O)cc8CC[C@@H]7c7ccccc7)cc6)CC5)OC4)CC3)C2=O)C(=O)N1] | (S)-3-(1′-(((S)-9-(4-((1R,2S)-6-hydroxy-2- phenyl-1,2,3,4-tetrahydronaphthalen-1- yl)phenyl)-1-oxa-9-azaspiro[5.5]undecan- 3-yl)methyl)-6-oxo-6,8-dihydro-2H,7H- spiro[furo[2,3-e]isoindole-3,4′-piperidin]- 7-yl)piperidine-2,6-dione |
| B401 | 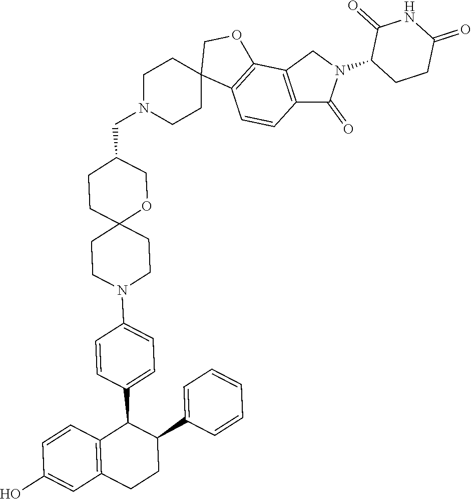 [SMILES: O=C1CC[C@H](N2Cc3c(ccc4c3OCC43CCN(C[C@H]4CCC5(CCN(c6ccc([C@@H]7c8ccc(O)cc8CC[C@@H]7c7ccccc7)cc6)CC5)OC4)CC3)C2=O)C(=O)N1] | (S)-3-(1′-(((R)-9-(4-((1R,2S)-6-hydroxy-2- phenyl-1,2,3,4-tetrahydronaphthalen-1- yl)phenyl)-1-oxa-9-azaspiro[5.5]undecan- 3-yl)methyl)-6-oxo-6,8-dihydro-2H,7H- spiro[furo[2,3-e]isoindole-3,4′-piperidin]- 7-yl)piperidine-2,6-dione |

TABLE 3
*designates that it is racemic with respect to the two stereogenic centers at the warhead (tetrahydronaphthalene); the relative
configuration of the two stereogenic center is cis.

| Compound No. | Chemical Structure | Chemical Name |
|---|---|---|
| B1* | 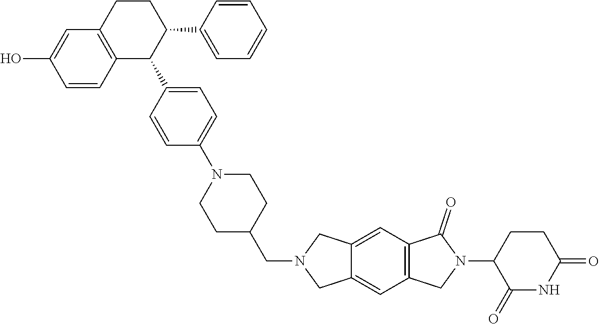 [SMILES: O=C1CCC(N2Cc3cc4c(cc3C2=O)CN(CC2CCN(c3ccc([C@@H]5c6ccc(O)cc6CC[C@@H]5c5ccccc5)cc3)CC2)C4)C(=O)N1] | 3-(6-((1-(4-((1R,2S)-6-hydroxy-2-phenyl-1,2,3,4- tetrahydronaphthalen-1-yl)phenyl)piperidin-4-yl)methyl)-1- oxo-3,5,6,7-tetrahydropyrrolo[3,4-f]isoindol-2(1H)- yl)piperidine-2,6-dione |
| B2* | 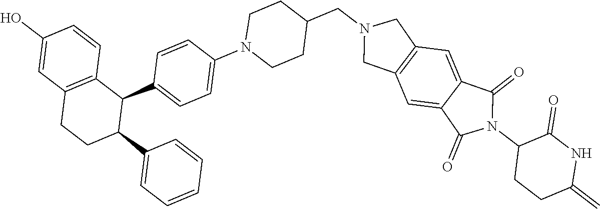 [SMILES: C=C1CCC(N2C(=O)c3cc4c(cc3C2=O)CN(CC2CCN(c3ccc([C@@H]5c6ccc(O)cc6CC[C@@H]5c5ccccc5)cc3)CC2)C4)C(=O)N1] | 2-(2,6-dioxopiperidin-3-yl)-6-((1-(4-((1R,2S)-6-hydroxy-2- phenyl-1,2,3,4-tetrahydronaphthalen-1-yl)phenyl)piperidin-4- yl)methyl)-6,7-dihydropyrrolo[3,4-f]isoindole-1,3(2H,5H)- dione |
| B3* | 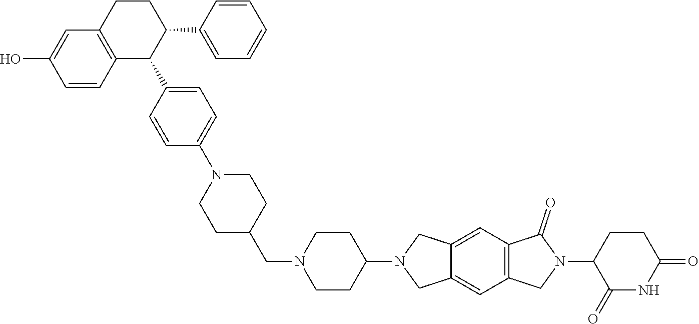 [SMILES: O=C1CCC(N2Cc3cc4c(cc3C2=O)CN(C2CCN(CC3CCN(c5ccc([C@@H]6c7ccc(O)cc7CC[C@@H]6c6ccccc6)cc5)CC3)CC2)C4)C(=O)N1] | 3-(6-(1-((1-(4-((1R,2S)-6-hydroxy-2-phenyl-1,2,3,4- tetrahydronaphthalen-1-yl)phenyl)piperidin-4- yl)methyl)piperidin-4-yl)-1-oxo-3,5,6,7-tetrahydropyrrolo[3,4- f]isoindol-2(1H)-yl)piperidine-2,6-dione |
| B4* | 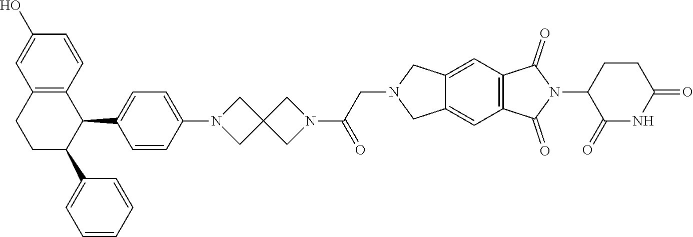 [SMILES: O=C1CCC(N2C(=O)c3cc4c(cc3C2=O)CN(CC(=O)N2CC3(C2)CN(c2ccc([C@@H]5c6ccc(O)cc6CC[C@@H]5c5ccccc5)cc2)C3)C4)C(=O)N1] | 2-(2,6-dioxopiperidin-3-yl)-6-(2-(6-(4-((1R,2S)-6-hydroxy-2- phenyl-1,2,3,4-tetrahydronaphthalen-1-yl)phenyl)-2,6- diazaspiro[3.3]heptan-2-yl)-2-oxoethyl)-6,7- dihydropyrrolo[3,4-f]isoindole-1,3(2H,5H)-dione |
| B5* | 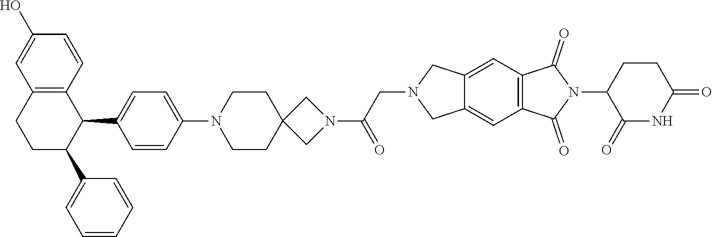 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(7-(4-((1R,2S)-6-hydroxy-2- phenyl-1,2,3,4-tetrahydronaphthalen-1-yl)phenyl)-2,7- diazaspiro[3.5]nonan-2-yl)-2-oxoethyl)-6,7- dihydropyrrolo[3,4-f]isoindole-1,3(2H,5H)-dione |
| B6* | 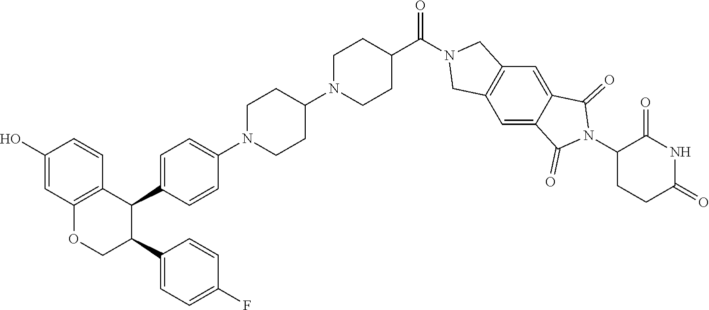 [SMILES: O=C1CCC(N2C(=O)c3cc4c(cc3C2=O)CN(C(=O)C2CCN(C3CCN(c5ccc([C@@H]6c7ccc(O)cc7OC[C@@H]6c6ccc(F)cc6)cc5)CC3)CC2)C4)C(=O)N1] | 2-(2,6-dioxopiperidin-3-yl)-6-(1′-(4-((1R,2S)-2-(4- fluorophenyl)-6-hydroxy-1,2,3,4-tetrahydronaphthalen-1- yl)phenyl)-[1,4'-bipiperidine]-4-carbonyl)-6,7- dihydropyrrolo[3,4-f]isoindole-1,3(2H,5H)-dione |
| B7 | 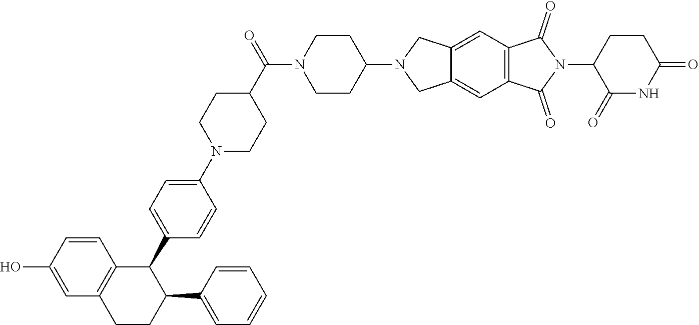 [SMILES: O=C1CCC(N2C(=O)c3cc4c(cc3C2=O)CN(C2CCN(C(=O)C3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-(1-(4-((1R,2S)-6-hydroxy-2- phenyl-1,2,3,4-tetrahydronaphthalen-1-yl)phenyl)piperidine-4- carbonyl)piperidin-4-yl)-6,7-dihydropyrrolo[3,4-f]isoindole- 1,3(2H,5H)-dione |
| B8* | 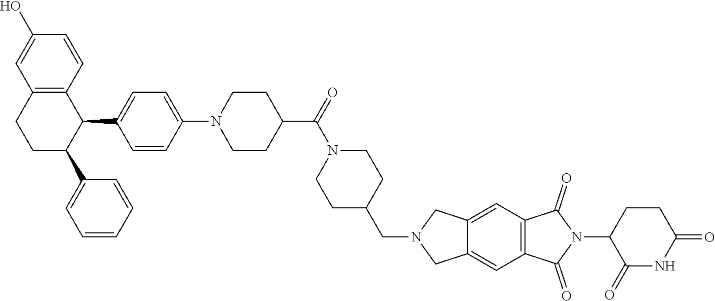 [SMILES: O=C1CCC(N2C(=O)c3cc4c(cc3C2=O)CN(CC2CCN(C(=O)C3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1-(1-(4-((1R,2S)-6-hydroxy-2- phenyl-1,2,3,4-tetrahydronaphthalen-1-yl)phenyl)piperidine-4- carbonyl)piperidin-4-yl)methyl)-6,7-dihydropyrrolo[3,4- f]isoindole-1,3(2H,5H)-dione |
| B9* | 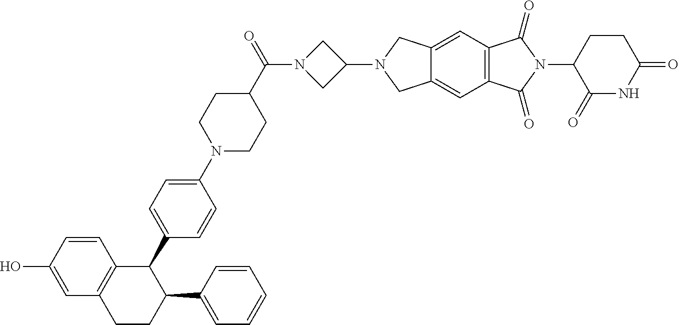 [SMILES: O=C1CCC(N2C(=O)c3cc4c(cc3C2=O)CN(C2CN(C(=O)C3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(1-(1-(4-((1R,2S)-6-hydroxy-2- phenyl-1,2,3,4-tetrahydronaphthalen-1-yl)phenyl)piperidine-4- carbonyl)azetidin-3-yl)-6,7-dihydropyrrolo[3,4-f]isoindole- 1,3(2H,5H)-dione |
| B10* | 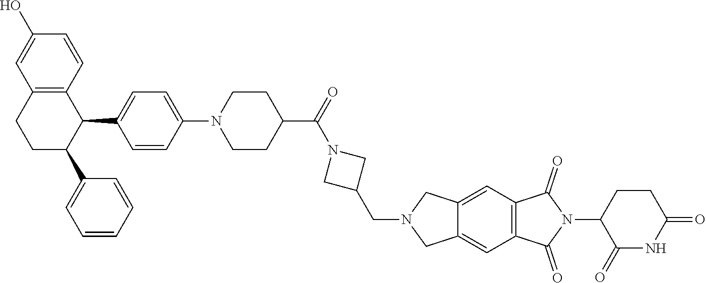 [SMILES: O=C1CCC(N2C(=O)c3cc4c(cc3C2=O)CN(CC2CN(C(=O)C3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((1-(1-(4-((1R,2S)-6-hydroxy- 2-phenyl-1,2,3,4-tetrahydronaphthalen-1- yl)phenyl)piperidine-4-carbonyl)azetidin-3-yl)methyl)-6,7- dihydropyrrolo[3,4-f]isoindole-1,3(2H,5H)-dione |
| B11* | 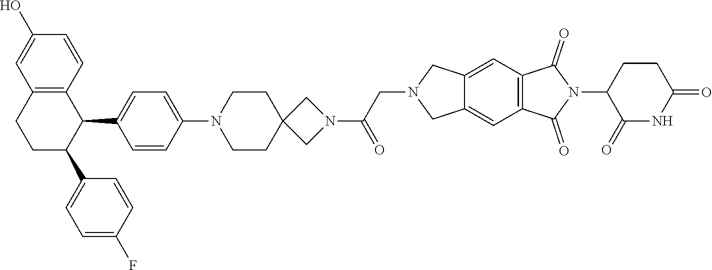 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccc(F)cc6)cc5)CC3)C2)C4)C(=O)N1] | 2-(2,6-dioxopiperidin-3-yl)-6-(2-(7-(4-((1R,2S)-2-(4- fluorophenyl)-6-hydroxy-1,2,3,4-tetrahydronaphthalen-1- yl)phenyl)-2,7-diazaspiro[3.5]nonan-2-yl)-2-oxoethyl)-6,7- dihydropyrrolo[3,4-f]isoindole-1,3(2H,5H)-dione |
| B12* | 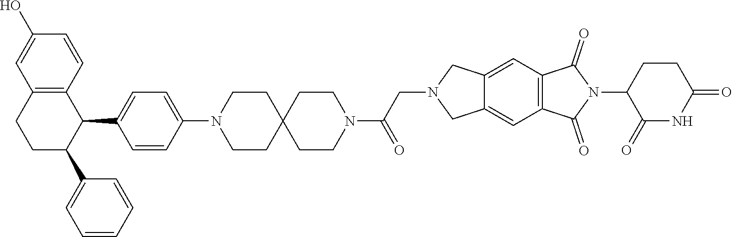 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)C4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)-2-oxoethyl)-6,7- dihydropyrrolo[3,4-f]isoindole-1,3(2H,5H)-dione |
| B13* | 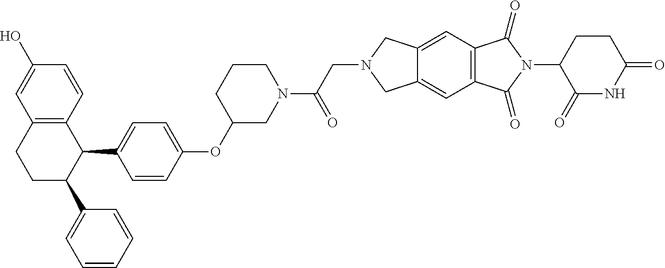 [SMILES: O=C1CCC(N2C(=O)c3cc4c(cc3C2=O)CN(CC(=O)N2CCCC(Oc3ccc([C@@H]5c6ccc(O)cc6CC[C@@H]5c5ccccc5)cc3)C2)C4)C(=O)N1] | 2-(2,6-dioxopiperidin-3-yl)-6-(2-(3-(4-((1R,2S)-6-hydroxy-2- phenyl-1,2,3,4-tetrahydronaphthalen-1-yl)phenoxy)piperidin- 1-yl)-2-oxoethyl)-6,7-dihydropyrrolo[3,4-f]isoindole- 1,3(2H,5H)-dione |
| B14* | 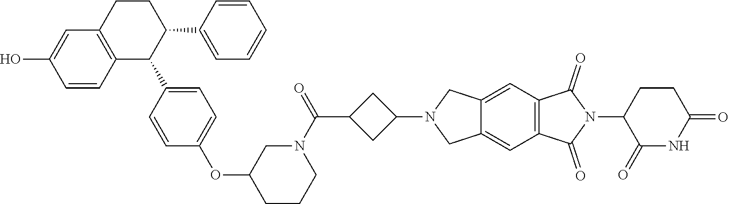 [SMILES: O=C1CCC(N2C(=O)c3cc4c(cc3C2=O)CN(C2CC(C(=O)N3CCCC(Oc5ccc([C@@H]6c7ccc(O)cc7CC[C@@H]6c6ccccc6)cc5)C3)C2)C4)C(=O)N1] | 2-(2,6-dioxopiperidin-3-yl)-6-(3-(3-(4-((1R,2S)-6-hydroxy-2- phenyl-1,2,3,4-tetrahydronaphthalen-1-yl)phenoxy)piperidine- 1-carbonyl)cyclobutyl)-6,7-dihydropyrrolo[3,4-f]isoindole- 1,3(2H,5H)-dione |
| B15* | 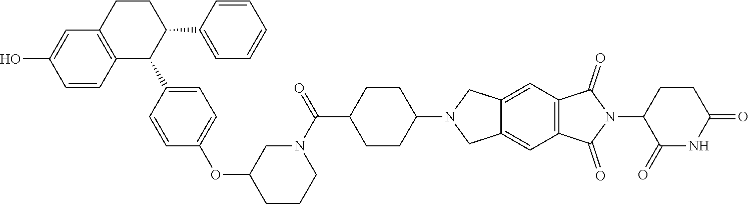 [SMILES: O=C1CCC(N2C(=O)c3cc4c(cc3C2=O)CN(C2CCC(C(=O)N3CCCC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-(4-(3-(4-((1R,2S)-6-hydroxy-2- phenyl-1,2,3,4-tetrahydronaphthalen-1-yl)phenoxy)piperidine- 1-carbonyl)cyclohexyl)-6,7-dihydropyrrolo[3,4-f]isoindole- 1,3(2H,5H)-dione |
| B16 | 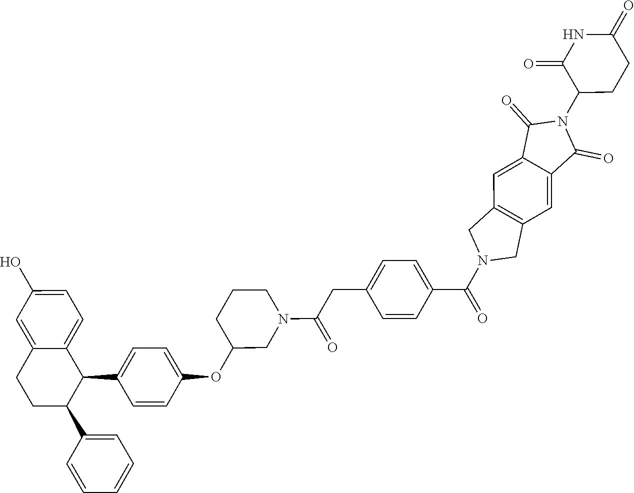 | 2-(2,6-dioxopiperidin-3-yl)-6-(4-(2-(3-(4-((1R,2S)-6-hydroxy- 2-phenyl-1,2,3,4-tetrahydronaphthalen-1-yl)phenoxy)piperidin- 1-yl)-2-oxoethyl)benzoyl)-6,7-dihydropyrrolo[3,4-f]isoindole- 1,3(2H,5H)-dione |
| B17* | 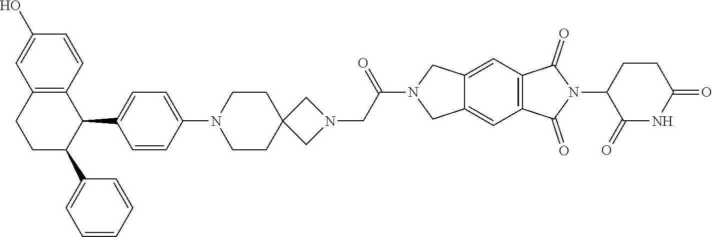 [SMILES: O=C1CCC(N2C(=O)c3cc4c(cc3C2=O)CN(C(=O)CN2CC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(7-(4-((1R,2S)-6-hydroxy-2- phenyl-1,2,3,4-tetrahydronaphthalen-1-yl)phenyl)-2,7- diazaspiro[3.5]nonan-2-yl)acetyl)-6,7-dihydropyrrolo[3,4- f]isoindole-1,3(2H,5H)-dione |
| B18* | 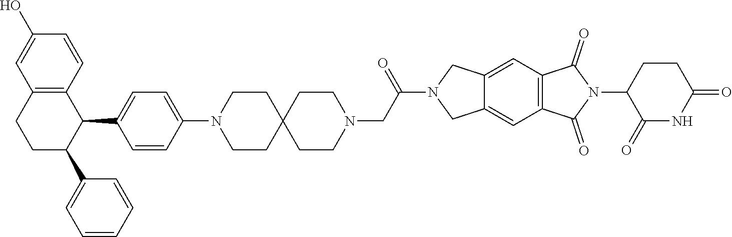 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)cc2)CC3)C4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)acetyl)-6,7-dihydropyrrolo[3,4- f]isoindole-1,3(2H,5H)-dione |
| B19* | 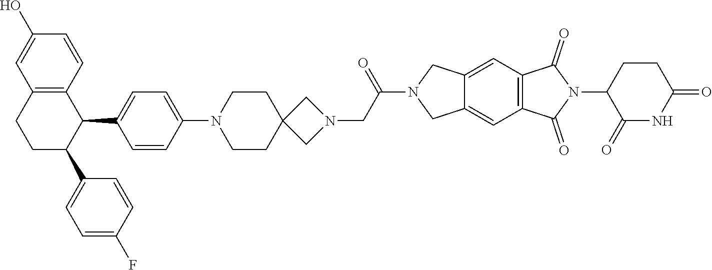 [SMILES: O=C1CCC(N2C(=O)c3cc4c(cc3C2=O)CN(C(=O)CN2CC3(CCN(c5ccc([C@@H]6c7ccc(O)cc7CC[C@@H]6c6ccc(F)cc6)cc5)CC3)C2)C4)C(=O)N1] | 2-(2,6-dioxopiperidin-3-yl)-6-(2-(7-(4-((1R,2S)-2-(4- fluorophenyl)-6-hydroxy-1,2,3,4-tetrahydronaphthalen-1- yl)phenyl)-2,7-diazaspiro[3.5]nonan-2-yl)acetyl)-6,7- dihydropyrrolo[3,4-f]isoindole-1,3(2H,5H)-dione |
| B20* | 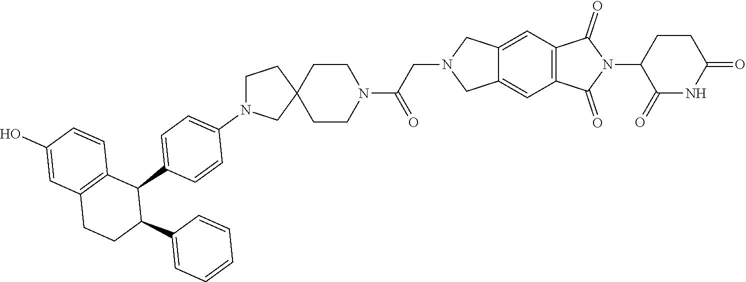 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4-((1R,2S)-6-hydroxy-2- phenyl-1,2,3,4-tetrahydronaphthalen-1-yl)phenyl)-2,8- diazaspiro[4.5]decan-8-yl)-2-oxoethyl)-6,7- dihydropyrrolo[3,4-f]isoindole-1,3(2H,5H)-dione |
| B21* | 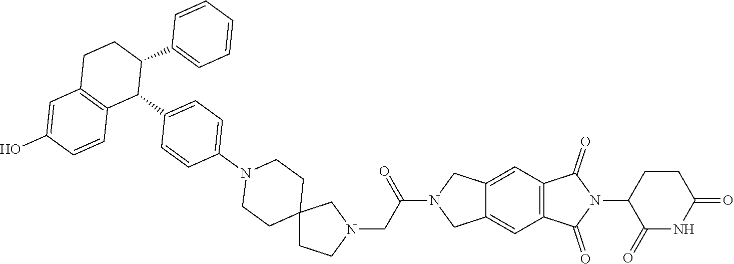 [SMILES: O=C1CCC(N2C(=O)c3cc4c(cc3C2=O)CN(C(=O)CN2CCC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(8-(4-((1R,2S)-6-hydroxy-2- phenyl-1,2,3,4-tetrahydronaphthalen-1-yl)phenyl)-2,8- diazaspiro[4.5]decan-2-yl)acetyl)-6,7-dihydropyrrolo[3,4- f]isoindole-1,3(2H,5H)-dione |
| B22* | 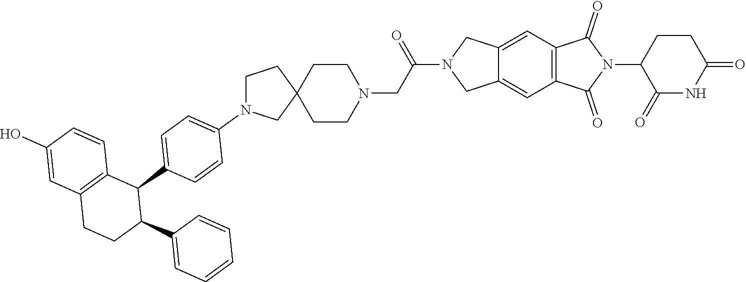 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4-((1R,2S)-6-hydroxy-2- phenyl-1,2,3,4-tetrahydronaphthalen-1-yl)phenyl)-2,8- diazaspiro[4.5]decan-8-yl)acetyl)-6,7-dihydropyrrolo[3,4- f]isoindole-1,3(2H,5H)-dione |
| B23* | 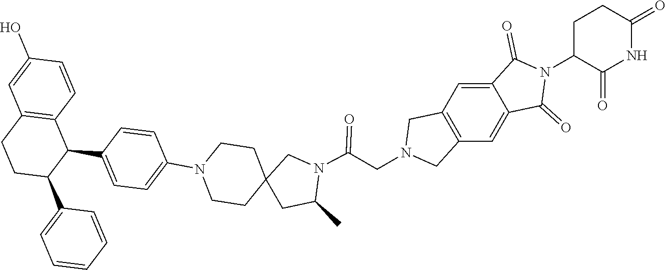 [SMILES: C[C@H]1CC2(CCN(c3ccc([C@@H]4c5ccc(O)cc5CC[C@@H]4c4ccccc4)cc3)CC2)CN1C(=O)CN1Cc2cc3c(cc2C1)C(=O)N(C1CCC(=O)NC1=O)C3=O] | 2-(2,6-dioxopiperidin-3-yl)-6-(2-((S)-8-(4-((1R,2S)-6-hydroxy- 2-phenyl-1,2,3,4-tetrahydronaphthalen-1-yl)phenyl)-3-methyl- 2,8-diazaspiro[4.5]decan-2-yl)-2-oxoethyl)-6,7- dihydropyrrolo[3,4-f]isoindole-1,3(2H,5H)-dione |
| B24* | 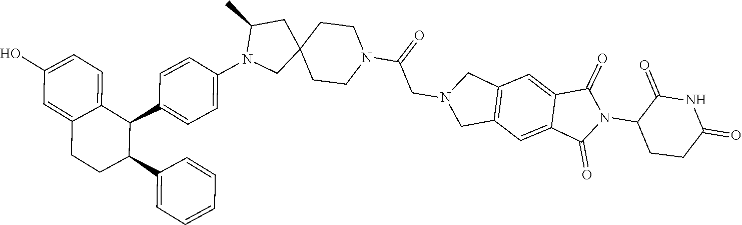 [SMILES: C[C@H]1CC2(CCN(C(=O)CN3Cc4cc5c(cc4C3)C(=O)N(C3CCC(=O)NC3=O)C5=O)CC2)CN1c1ccc([C@@H]2c3ccc(O)cc3CC[C@@H]2c2ccccc2)cc1] | 2-(2,6-dioxopiperidin-3-yl)-6-(2-((S)-2-(4-((1R,2S)-6-hydroxy- 2-phenyl-1,2,3,4-tetrahydronaphthalen-1-yl)phenyl)-3-methyl- 2,8-diazaspiro[4.5]decan-8-yl)-2-oxoethyl)-6,7- dihydropyrrolo[3,4-f]isoindole-1,3(2H,5H)-dione |
| B25* | 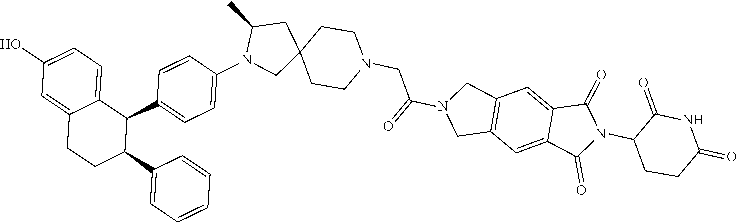 [SMILES: C[C@H]1CC2(CCN(CC(=O)N3Cc4cc5c(cc4C3)C(=O)N(C3CCC(=O)NC3=O)C5=O)CC2)CN1c1ccc([C@@H]2c3ccc(O)cc3CC[C@@H]2c2ccccc2)cc1] | 2-(2,6-dioxopiperidin-3-yl)-6-(2-((S)-2-(4-((1R,2S)-6-hydroxy- 2-phenyl-1,2,3,4-tetrahydronaphthalen-1-yl)phenyl)-3-methyl- 2,8-diazaspiro[4.5]decan-8-yl)acetyl)-6,7-dihydropyrrolo[3,4- f]isoindole-1,3(2H,5H)-dione |
| B26* | 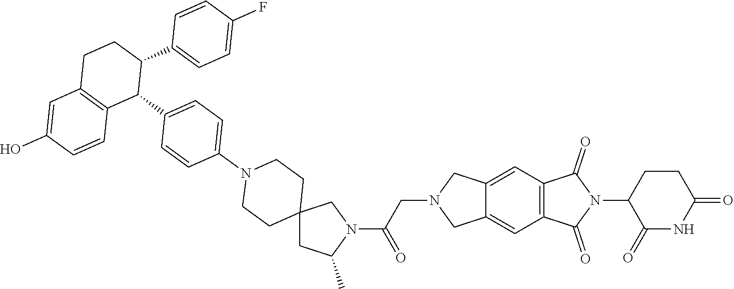 [SMILES: C[C@@H]1CC2(CCN(c3ccc([C@@H]4c5ccc(O)cc5CC[C@@H]4c4ccc(F)cc4)cc3)CC2)CN1C(=O)CN1Cc2cc3c(cc2C1)C(=O)N(C1CCC(=O)NC1=O)C3=O] | 2-(2,6-dioxopiperidin-3-yl)-6-(2-((R)-8-(4-((1R,2S)-2-(4- fluorophenyl)-6-hydroxy-1,2,3,4-tetrahydronaphthalen-1- yl)phenyl)-3-methyl-2,8-diazaspiro[4.5]decan-2-yl)-2- oxoethyl)-6,7-dihydropyrrolo[3,4-f]isoindole-1,3(2H,5H)- dione |
| B27* | 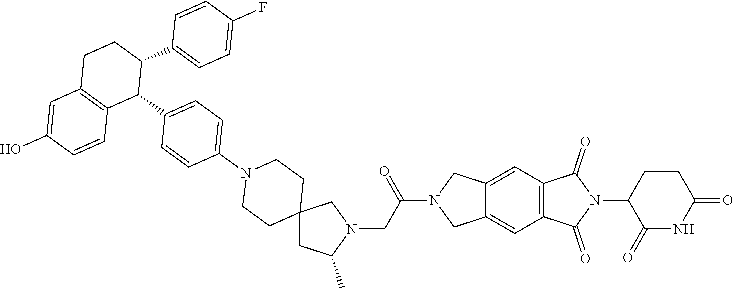 [SMILES: C[C@@H]1CC2(CCN(c3ccc([C@@H]4c5ccc(O)cc5CC[C@@H]4c4ccc(F)cc4)cc3)CC2)CN1CC(=O)N1Cc2cc3c(cc2C1)C(=O)N(C1CCC(=O)NC1=O)C3=O] | 2-(2,6-dioxopiperidin-3-yl)-6-(2-((R)-8-(4-((1R,2S)-2-(4- fluorophenyl)-6-hydroxy-1,2,3,4-tetrahydronaphthalen-1- yl)phenyl)-3-methyl-2,8-diazaspiro[4.5]decan-2-yl)acetyl)-6,7- dihydropyrrolo[3,4-f]isoindole-1,3(2H,5H)-dione |
| B28 | 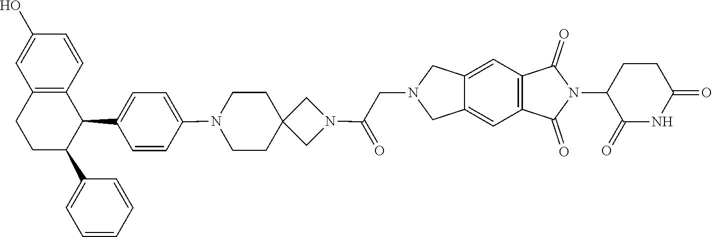 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(7-(4-((1R,2S)-6-hydroxy-2- phenyl-1,2,3,4-tetrahydronaphthalen-1-yl)phenyl)-2,7- diazaspiro[3.5]nonan-2-yl)-2-oxoethyl)-6,7- dihydropyrrolo[3,4-f]isoindole-1,3(2H,5H)-dione |
| B29* | 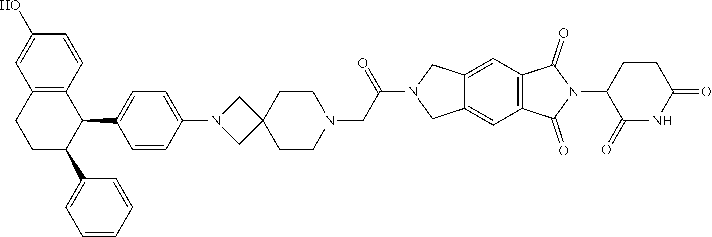 [SMILES: O=C1CCC(N2C(=O)c3cc4c(cc3C2=O)CN(C(=O)CN2CCC3(CC2)CN(c2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4-((1R,2S)-6-hydroxy-2- phenyl-1,2,3,4-tetrahydronaphthalen-1-yl)phenyl)-2,7- diazaspiro[3.5]nonan-7-yl)acetyl)-6,7-dihydropyrrolo[3,4- f]isoindole-1,3(2H,5H)-dione |
| B30* | 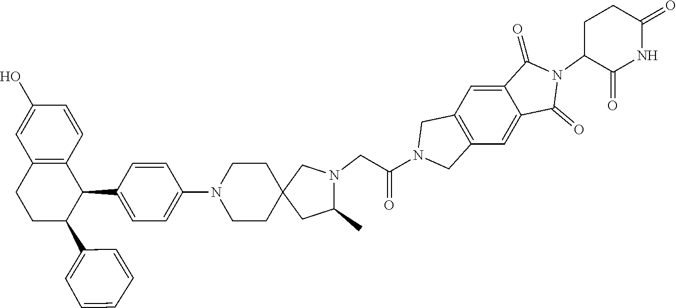 [SMILES: C[C@H]1CC2(CCN(c3ccc([C@@H]4c5ccc(O)cc5CC[C@@H]4c4ccccc4)cc3)CC2)CN1CC(=O)N1Cc2cc3c(cc2C1)C(=O)N(C1CCC(=O)NC1=O)C3=O] | 2-(2,6-dioxopiperidin-3-yl)-6-(2-((S)-8-(4-((1R,2S)-6-hydroxy- 2-phenyl-1,2,3,4-tetrahydronaphthalen-1-yl)phenyl)-3-methyl- 2,8-diazaspiro[4.5]decan-2-yl)acetyl)-6,7-dihydropyrrolo[3,4- f]isoindole-1,3(2H,5H)-dione |
| B31* | 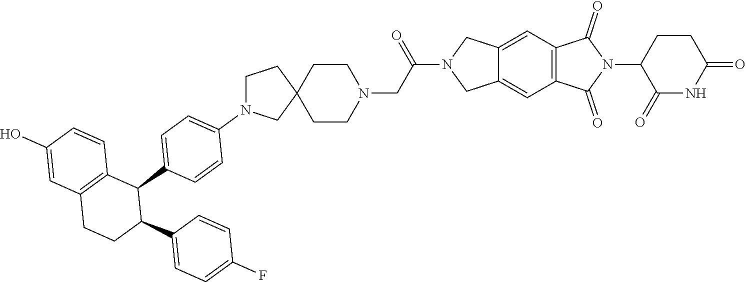 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccc(F)cc5)cc2)C3)C4)C(=O)N1] | 2-(2,6-dioxopiperidin-3-yl)-6-(2-(2-(4-((1R,2S)-2-(4- fluorophenyl)-6-hydroxy-1,2,3,4-tetrahydronaphthalen-1- yl)phenyl)-2,8-diazaspiro[4.5]decan-8-yl)acetyl)-6,7- dihydropyrrolo[3,4-f]isoindole-1,3(2H,5H)-dione |
| B32* | 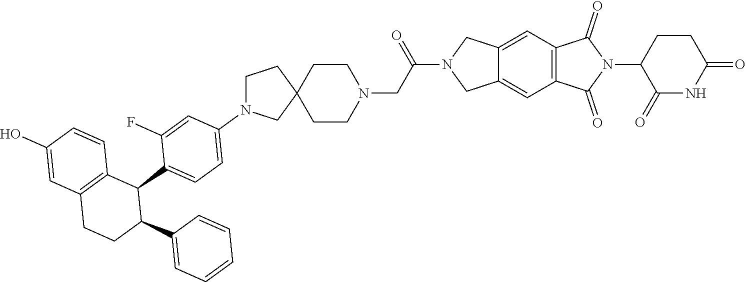 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)c(F)c2)C3)C4)C(=O)N1] | 2-(2,6-dioxopiperidin-3-yl)-6-(2-(2-(3-fluoro-4-((1S,2S)-6- hydroxy-2-phenyl-1,2,3,4-tetrahydronaphthalen-1-yl)phenyl)- 2,8-diazaspiro[4.5]decan-8-yl)acetyl)-6,7-dihydropyrrolo[3,4- f]isoindole-1,3(2H,5H)-dione |
| B33* | 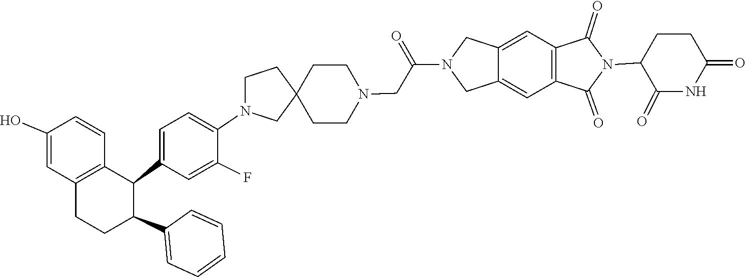 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)cc2F)C3)C4)C(=O)N1] | 2-(2,6-dioxopiperidin-3-yl)-6-(2-(2-(2-fluoro-4-((1R,2S)-6- hydroxy-2-phenyl-1,2,3,4-tetrahydronaphthalen-1-yl)phenyl)- 2,8-diazaspiro[4.5]decan-8-yl)acetyl)-6,7-dihydropyrrolo[3,4- f]isoindole-1,3(2H,5H)-dione |
| B34* | 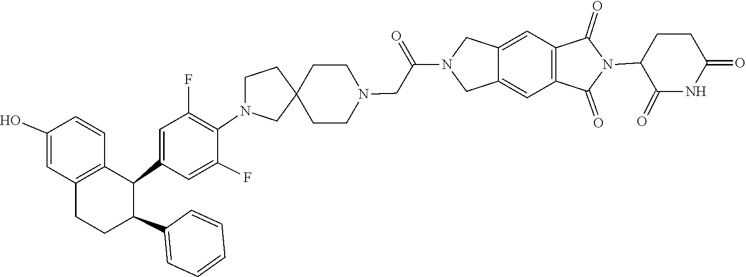 [SMILES: O=C1CCC(N2C(=O)c3cc4c(cc3C2=O)CN(C(=O)CN2CCC3(CC2)CCN(c2c(F)cc([C@@H]5c6ccc(O)cc6CC[C@@H]5c5ccccc5)cc2F)C3)C4)C(=O)N1] | 6-(2-(2-(2,6-difluoro-4-((1R,2S)-6-hydroxy-2-phenyl-1,2,3,4- tetrahydronaphthalen-1-yl)phenyl)-2,8-diazaspiro[4.5]decan-8- yl)acetyl)-2-(2,6-dioxopiperidin-3-yl)-6,7-dihydropyrrolo[3,4- f]isoindole-1,3(2H,5H)-dione |
| B35* | 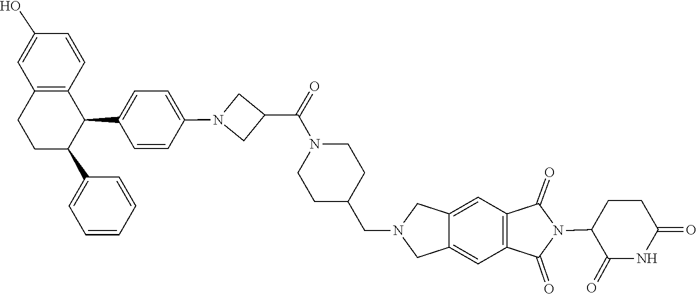 [SMILES: O=C1CCC(N2C(=O)c3cc4c(cc3C2=O)CN(CC2CCN(C(=O)C3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-(1-(4-((1R,2S)-6-hydroxy-2- phenyl-1,2,3,4-tctrahydronaphthalcn-1-yl)phcnyl)azctidinc-3- carbonyl)piperidin-4-yl)methyl)-6,7-dihydropyrrolo[3,4- f]isoindole-1,3(2H,5H)-dione |
| B36* | 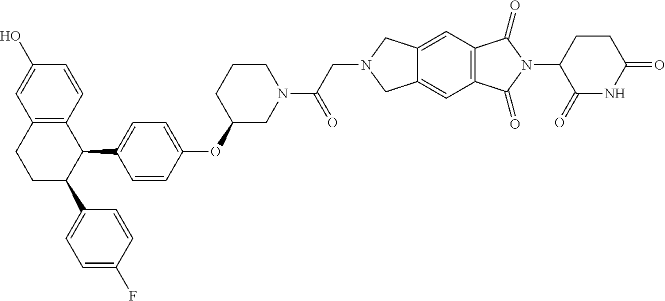 [SMILES: O=C1CCC(N2C(=O)c3cc4c(cc3C2=O)CN(CC(=O)N2CCC[C@H](Oc3ccc([C@@H]5c6ccc(O)cc6CC[C@@H]5c5ccc(F)cc5)cc3)C2)C4)C(=O)N1] | 2-(2,6-dioxopiperidin-3-yl)-6-(2-((S)-3-(4-((1R,2S)-2-(4- fluorophenyl)-6-hydroxy-1,2,3,4-tetrahydronaphthalen-1- yl)phenoxy)piperidin-1-yl)-2-oxoethyl)-6,7- dihydropyrrolo[3,4-f]isoindole-1,3(2H,5H)-dione |
| B37* | 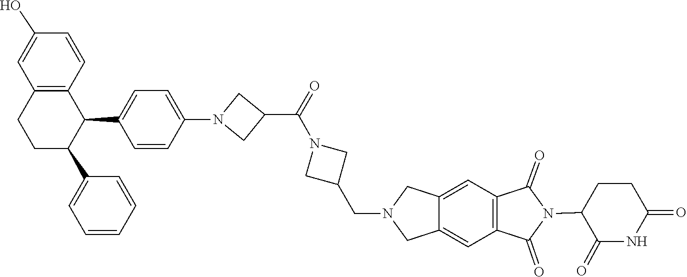 [SMILES: O=C1CCC(N2C(=O)c3cc4c(cc3C2=O)CN(CC2CN(C(=O)C3CN(c5ccc([C@@H]6c7ccc(O)cc7CC[C@@H]6c6ccccc6)cc5)C3)C2)C4)C(=O)N1] | 2-(2,6-dioxopiperidin-3-yl)-6-((1-(1-(4-((1R,2S)-6-hydroxy-2- phenyl-1,2,3,4-tetrahydronaphthalen-1-yl)phenyl)azetidine-3- carbonyl)azetidin-3-yl)methyl)-6,7-dihydropyrrolo[3,4- f]isoindole-1,3(2H,5H)-dione |
| B38* | 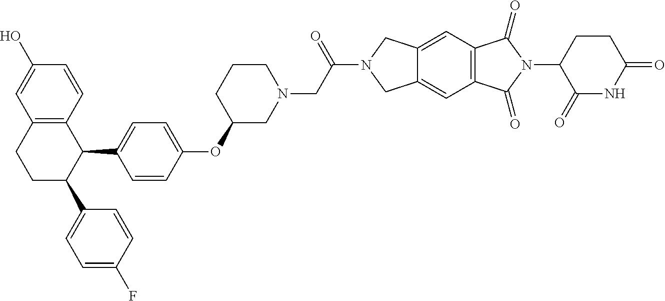 [SMILES: O=C1CCC(N2C(=O)c3cc4c(cc3C2=O)CN(C(=O)CN2CCC[C@H](Oc3ccc([C@@H]5c6ccc(O)cc6CC[C@@H]5c5ccc(F)cc5)cc3)C2)C4)C(=O)N1] | 2-(2,6-dioxopiperidin-3-yl)-6-(2-((S)-3-(4-((1R,2S)-2-(4- fluorophenyl)-6-hydroxy-1,2,3,4-tetrahydronaphthalen-1- yl)phenoxy)piperidin-1-yl)acetyl)-6,7-dihydropyrrolo[3,4- f]isoindole-1,3(2H,5H)-dione |
| B39 | 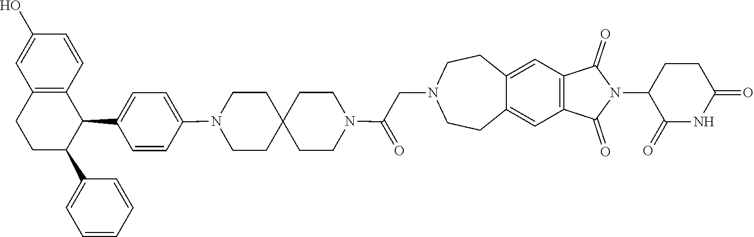 [SMILES: O=C1CCC(N2C(=O)c3cc4c(cc3C2=O)CCN(CC(=O)N2CCC3(CC2)CCN(c2ccc([C@@H]5c6ccc(O)cc6CC[C@@H]5c5ccccc5)cc2)CC3)CC4)C(=O)N1] | 2-(2,6-dioxopiperidin-3-yl)-7-(2-(9-(4-((1R,2S)-6-hydroxy-2- phenyl-1,2,3,4-tetrahydronaphthalen-1-yl)phenyl)-3,9- diazaspiro[5.5]undecan-3-yl)-2-oxoethyl)-6,7,8,9- tetrahydroazepino[4,5-f]isoindole-1,3(2H,5H)-dione |
| B40 | 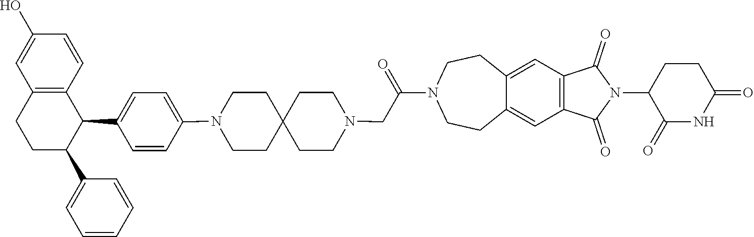 [SMILES: O=C1CCC(N2C(=O)c3cc4c(cc3C2=O)CCN(C(=O)CN2CCC3(CC2)CCN(c2ccc([C@@H]5c6ccc(O)cc6CC[C@@H]5c5ccccc5)cc2)CC3)CC4)C(=O)N1] | 2-(2,6-dioxopiperidin-3-yl)-7-(2-(9-(4-((1R,2S)-6-hydroxy-2- phenyl-1,2,3,4-tetrahydronaphthalen-1-yl)phenyl)-3,9- diazaspiro[5.5]undecan-3-yl)acetyl)-6,7,8,9- tetrahydroazepino[4,5-f]isoindole-1,3(2H,5H)-dione |
| B41 | 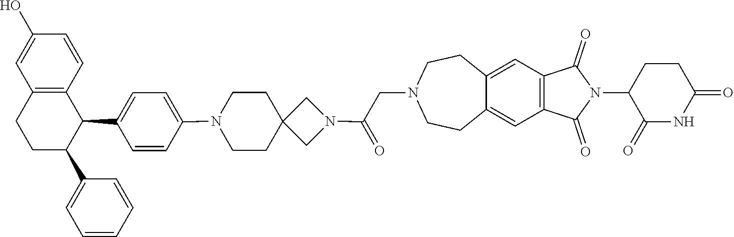 [SMILES: O=C1CCC(N2C(=O)c3cc4c(cc3C2=O)CCN(CC(=O)N2CC3(CCN(c5ccc([C@@H]6c7ccc(O)cc7CC[C@@H]6c6ccccc6)cc5)CC3)C2)CC4)C(=O)N1] | 2-(2,6-dioxopiperidin-3-yl)-7-(2-(7-(4-((1R,2S)-6-hydroxy-2- phenyl-1,2,3,4-tetrahydronaphthalen-1-yl)phenyl)-2,7- diazaspiro[3.5]nonan-2-yl)-2-oxoethyl)-6,7,8,9- tetrahydroazepino[4,5-f]isoindole-1,3(2H,5H)-dione |
| B42 | 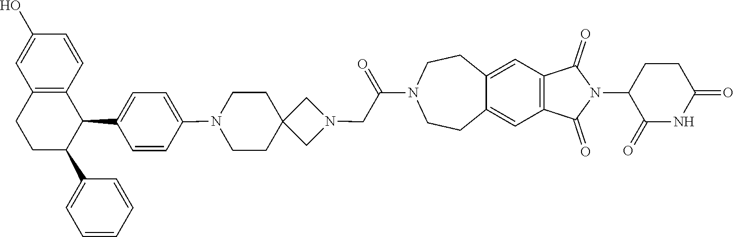 [SMILES: O=C1CCC(N2C(=O)c3cc4c(cc3C2=O)CCN(C(=O)CN2CC3(CCN(c5ccc([C@@H]6c7ccc(O)cc7CC[C@@H]6c6ccccc6)cc5)CC3)C2)CC4)C(=O)N1] | 2-(2,6-dioxopiperidin-3-yl)-7-(2-(7-(4-((1R,2S)-6-hydroxy-2- phenyl-1,2,3,4-tetrahydronaphthalen-1-yl)phenyl)-2,7- diazaspiro[3.5]nonan-2-yl)acetyl)-6,7,8,9- tetrahydroazepino[4,5-f]isoindole-1,3(2H,5H)-dione |
| B43* | 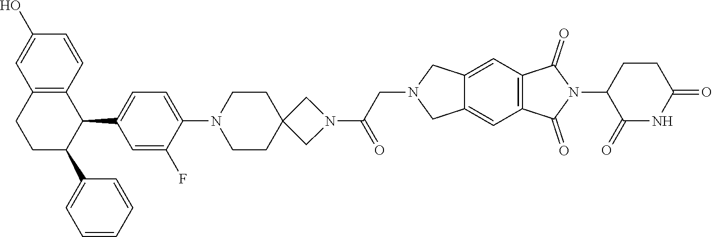 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5F)CC3)C2)C4)C(=O)N1] | 2-(2,6-dioxopiperidin-3-yl)-6-(2-(7-(2-fluoro-4-((1R,2S)-6- hydroxy-2-phenyl-1,2,3,4-tetrahydronaphthalen-1-yl)phenyl)- 2,7-diazaspiro[3.5]nonan-2-yl)-2-oxoethyl)-6,7- dihydropyrrolo[3,4-f]isoindole-1,3(2H,5H)-dione |
| B44* | 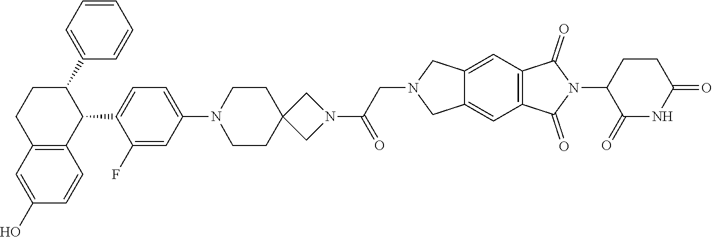 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)c(F)c5)CC3)C2)C4)C(=O)N1] | 2-(2,6-dioxopiperidin-3-yl)-6-(2-(7-(3-fluoro-4-((1S,2S)-6- hydroxy-2-phenyl-1,2,3,4-tetrahydronaphthalen-1-yl)phenyl)- 2,7-diazaspiro[3.5]nonan-2-yl)-2-oxoethyl)-6,7- dihydropyrrolo[3,4-f]isoindole-1,3(2H,5H)-dione |
| B45* | 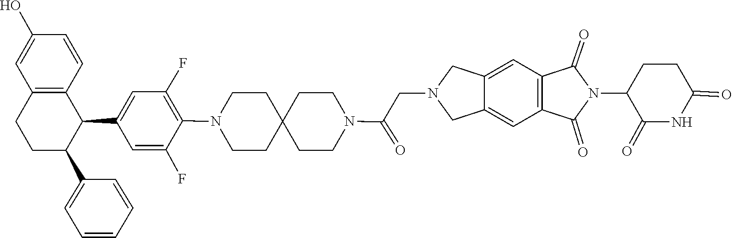 [SMILES: O=C1CCC(N2C(=O)c3cc4c(cc3C2=O)CN(CC(=O)N2CCC3(CC2)CCN(c2c(F)cc([C@@H]5c6ccc(O)cc6CC[C@@H]5c5ccccc5)cc2F)CC3)C4)C(=O)N1] | 6-(2-(9-(2,6-difluoro-4-((1R,2S)-6-hydroxy-2-phenyl-1,2,3,4- tetrahydronaphthalen-1-yl)phenyl)-3,9-diazaspiro[5.5]undecan- 3-yl)-2-oxoethyl)-2-(2,6-dioxopiperidin-3-yl)-6,7- dihydropyrrolo[3,4-f]isoindole-1,3(2H,5H)-dione |
| B46* | 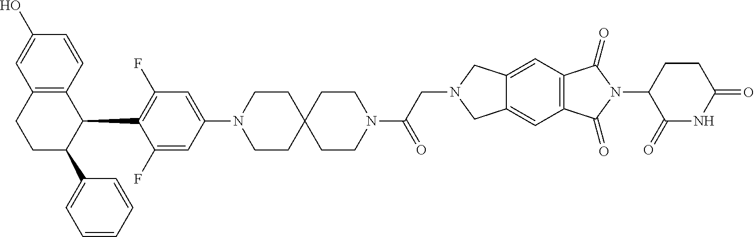 [SMILES: O=C1CCC(N2C(=O)c3cc4c(cc3C2=O)CN(CC(=O)N2CCC3(CC2)CCN(c2cc(F)c([C@@H]5c6ccc(O)cc6CC[C@@H]5c5ccccc5)c(F)c2)CC3)C4)C(=O)N1] | 6-(2-(9-(3,5-difluoro-4-((1S,2S)-6-hydroxy-2-phenyl-1,2,3,4- tetrahydronaphthalen-1-yl)phenyl)-3,9-diazaspiro[5.5]undecan- 3-yl)-2-oxoethyl)-2-(2,6-dioxopiperidin-3-yl)-6,7- dihydropyrrolo[3,4-f]isoindole-1,3(2H,5H)-dione |
| B47* | 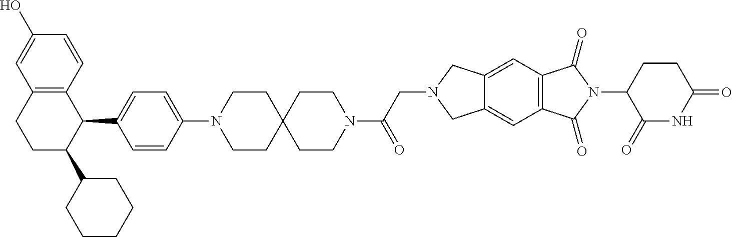 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5C5CCCCC5)cc2)CC3)C4)C(=O)N1] | 6-(2-(9-(4-((1R,2R)-2-cyclohexyl-6-hydroxy-1,2,3,4- tetrahydronaphthalen-1-yl)phenyl)-3,9-diazaspiro[5.5]undecan- 3-yl)-2-oxoethyl)-2-(2,6-dioxopiperidin-3-yl)-6,7- dihydropyrrolo[3,4-f]isoindole-1,3(2H,5H)-dione |
| B48* | 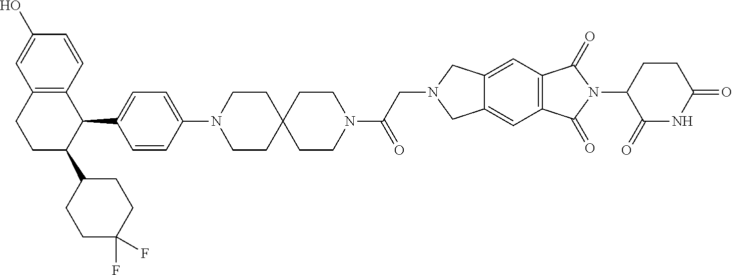 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5C5CCC(F)(F)CC5)cc2)CC3)C4)C(=O)N1] | 6-(2-(9-(4-((1R,2R)-2-(4,4-difluorocyclohexyl)-6-hydroxy- 1,2,3,4-tetrahydronaphthalen-1-yl)phenyl)-3,9- diazaspiro[5.5]undecan-3-yl)-2-oxoethyl)-2-(2,6- dioxopiperidin-3-yl)-6,7-dihydropyrrolo[3,4-f]isoindole- 1,3(2H,5H)-dione |
| B49 | 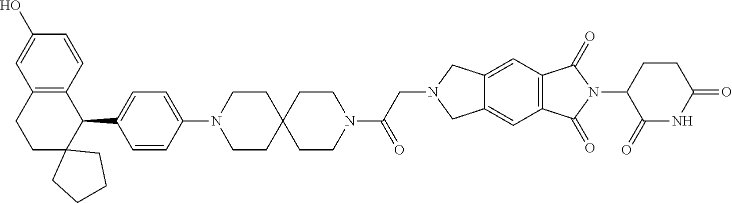 [SMILES: O=C1CCC(N2C(=O)c3cc4c(cc3C2=O)CN(CC(=O)N2CCC3(CC2)CCN(c2ccc([C@@H]5c6ccc(O)cc6CCC56CCCC6)cc2)CC3)C4)C(=O)N1] | 2-(2,6-dioxopiperidin-3-yl)-6-(2-(9-(4-(6'-hydroxy-3',4'- dihydro-1'H-spiro[cyclopentane-1,2'-naphthalen]-1'-yl)phenyl)- 3,9-diazaspiro[5.5]undecan-3-yl)-2-oxoethyl)-6,7- dihydropyrrolo[3,4-f]isoindole-1,3(2H,5H)-dione |
| B50* | 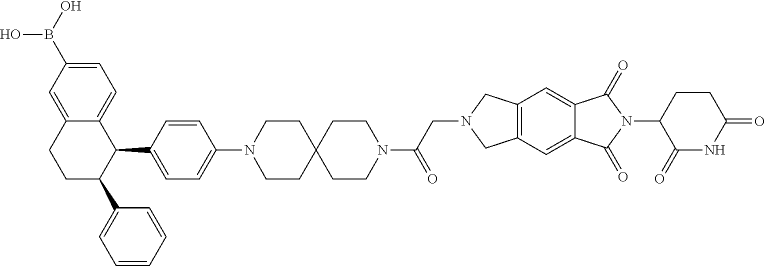 [SMILES: O=C1CCC(N2C(=O)c3cc4c(cc3C2=O)CN(CC(=O)N2CCC3(CC2)CCN(c2ccc([C@@H]5c6ccc(B(O)O)cc6CC[C@@H]5c5ccccc5)cc2)CC3)C4)C(=O)N1] | ((5R,6S)-5-(4-(9-(2-(6-(2,6-dioxopiperidin-3-yl)-5,7-dioxo- 3,5,6,7-tetrahydropyrrolo[3,4-f]isoindol-2(1H)-yl)acetyl)-3,9- diazaspiro[5.5]undecan-3-yl)phenyl)-6-phenyl-5,6,7,8- tetrahydronaphthalen-2-yl)boronic acid |
| B51* | 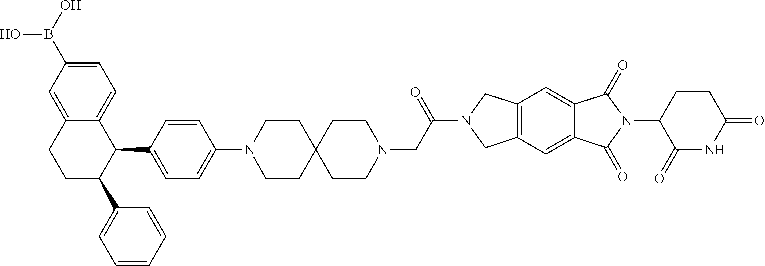 [SMILES: O=C1CCC(N2C(=O)c3cc4c(cc3C2=O)CN(C(=O)CN2CCC3(CC2)CCN(c2ccc([C@@H]5c6ccc(B(O)O)cc6CC[C@@H]5c5ccccc5)cc2)CC3)C4)C(=O)N1] | ((5R,6S)-5-(4-(9-(2-(6-(2,6-dioxopiperidin-3-yl)-5,7-dioxo- 3,5,6,7-tetrahydropyrrolo[3,4-f]isoindol-2(1H)-yl)-2-oxoethyl)- 3,9-diazaspiro[5.5]undecan-3-yl)phenyl)-6-phenyl-5,6,7,8- tetrahydronaphthalen-2-yl)boronic acid |
| B52 | 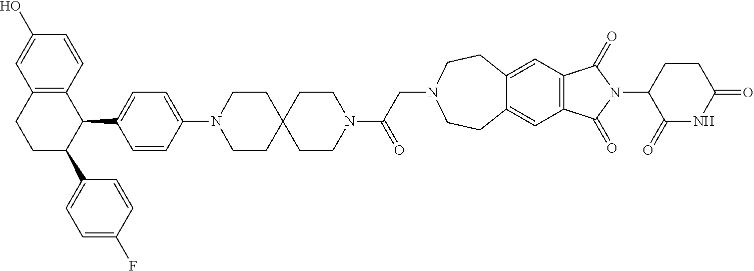 [SMILES: O=C1CCC(N2C(=O)c3cc4c(cc3C2=O)CCN(CC(=O)N2CCC3(CC2)CCN(c2ccc([C@@H]5c6ccc(O)cc6CC[C@@H]5c5ccc(F)cc5)cc2)CC3)CC4)C(=O)N1] | 2-(2,6-dioxopiperidin-3-yl)-7-(2-(9-(4-((1R,2S)-2-(4- fluorophenyl)-6-hydroxy-1,2,3,4-tetrahydronaphthalen-1- yl)phenyl)-3,9-diazaspiro[5.5]undecan-3-yl)-2-oxoethyl)- 6,7,8,9-tetrahydroazepino[4,5-f]isoindole-1,3(2H,5H)-dione |
| B53 | 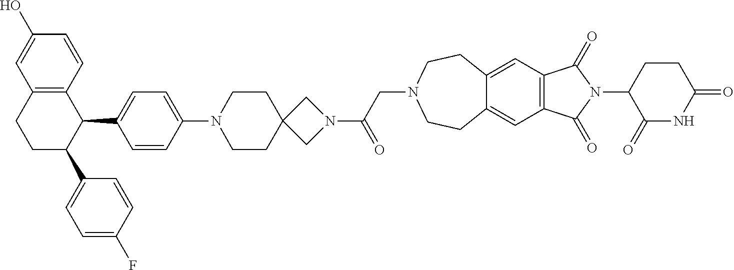 [SMILES: O=C1CCC(N2C(=O)c3cc4c(cc3C2=O)CCN(CC(=O)N2CC3(CCN(c5ccc([C@@H]6c7ccc(O)cc7CC[C@@H]6c6ccc(F)cc6)cc5)CC3)C2)CC4)C(=O)N1] | 2-(2,6-dioxopiperidin-3-yl)-7-(2-(7-(4-((1R,2S)-2-(4- fluorophenyl)-6-hydroxy-1,2,3,4-tetrahydronaphthalen-1- yl)phenyl)-2,7-diazaspiro[3.5]nonan-2-yl)-2-oxoethyl)-6,7,8,9- tetrahydroazepino[4,5-f]isoindole-1,3(2H,5H)-dione |
| B54 | 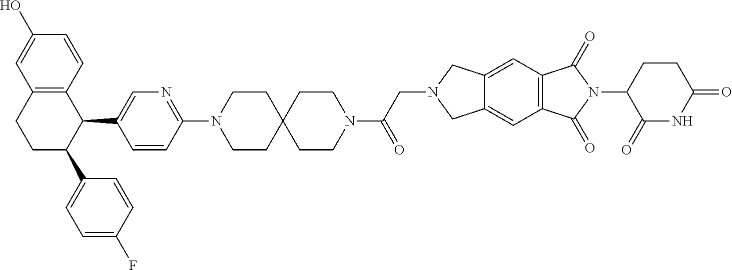 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccc(F)cc5)cn2)CC3)C4)C(=O)N1] | 2-(2,6-dioxopiperidin-3-yl)-6-(2-(9-(5-((1S,2S)-2-(4- fluorophenyl)-6-hydroxy-1,2,3,4-tetrahydronaphthalen-1- yl)pyridin-2-yl)-3,9-diazaspiro[5.5]undecan-3-yl)-2-oxoethyl)- 6,7-dihydropyrrolo[3,4-f]isoindole-1,3(2H,5H)-dione |
| B55 | 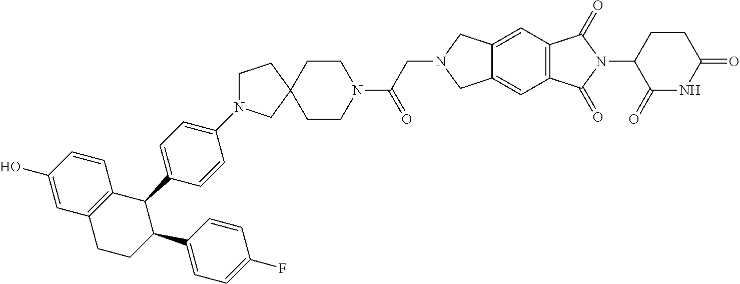 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccc(F)cc5)cc2)C3)C4)C(=O)N1] | 2-(2,6-dioxopiperidin-3-yl)-6-(2-(2-(4-((1R,2S)-2-(4- fluorophenyl)-6-hydroxy-1,2,3,4-tetrahydronaphthalen-1- yl)phenyl)-2,8-diazaspiro[4.5]decan-8-yl)-2-oxoethyl)-6,7- dihydropyrrolo[3,4-f]isoindole-1,3(2H,5H)-dione |
| B56 | 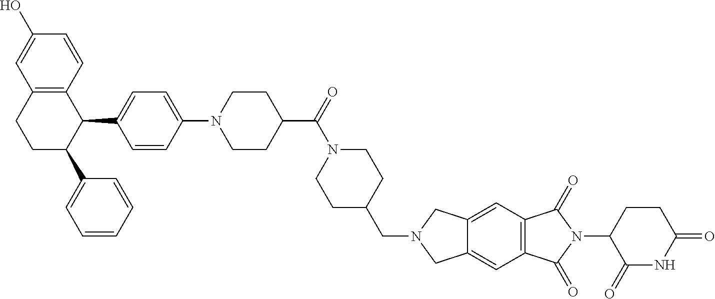 [SMILES: O=C1CCC(N2C(=O)c3cc4c(cc3C2=O)CN(CC2CCN(C(=O)C3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1-(1-(4-((1R,2S)-6-hydroxy-2- phenyl-1,2,3,4-tetrahydronaphthalen-1-yl)phenyl)piperidine-4- carbonyl)piperidin-4-yl)methyl)-6,7-dihydropyrrolo[3,4- f]isoindole-1,3(2H,5H)-dione |
| B57 | 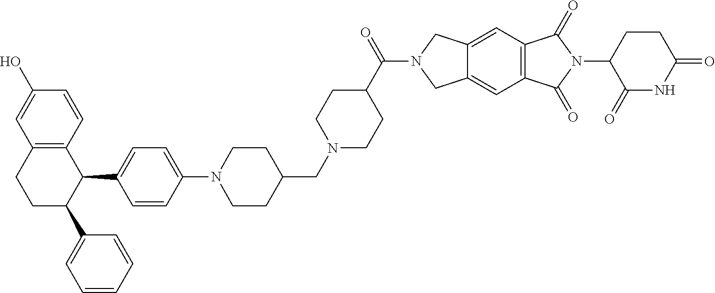 [SMILES: O=C1CCC(N2C(=O)c3cc4c(cc3C2=O)CN(C(=O)C2CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-((1-(4-((1R,2S)-6-hydroxy-2- phenyl-1,2,3,4-tetrahydronaphthalen-1-yl)phenyl)piperidin-4- yl)methyl)piperidine-4-carbonyl)-6,7-dihydropyrrolo[3,4- f]isoindole-1,3(2H,5H)-dione |
| B58 | 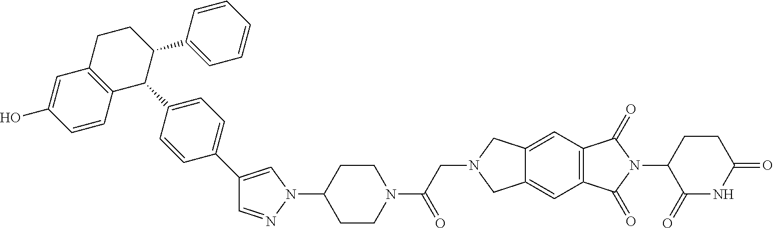 [SMILES: O=C1CCC(N2C(=O)c3cc4c(cc3C2=O)CN(CC(=O)N2CCC(n3cc(-c5ccc([C@@H]6c7ccc(O)cc7CC[C@@H]6c6ccccc6)cc5)cn3)CC2)C4)C(=O)N1] | 2-(2,6-dioxopiperidin-3-yl)-6-(2-(4-(4-(4-((1R,2S)-6-hydroxy- 2-phenyl-1,2,3,4-tetrahydronaphthalen-1-yl)phenyl)-1H- pyrazol-1-yl)piperidin-1-yl)-2-oxoethyl)-6,7- dihydropyrrolo[3,4-f]isoindole-1,3(2H,5H)-dione |
| B59 | 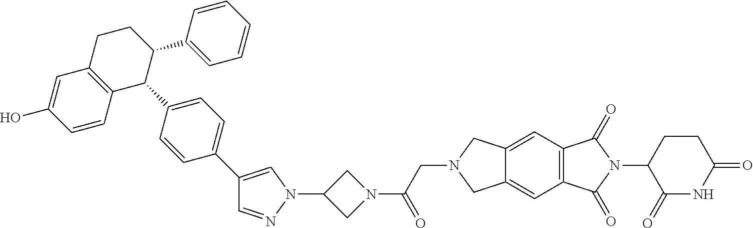 [SMILES: O=C1CCC(N2C(=O)c3cc4c(cc3C2=O)CN(CC(=O)N2CC(n3cc(-c5ccc([C@@H]6c7ccc(O)cc7CC[C@@H]6c6ccccc6)cc5)cn3)C2)C4)C(=O)N1] | 2-(2,6-dioxopiperidin-3-yl)-6-(2-(3-(4-(4-((1R,2S)-6-hydroxy- 2-phenyl-1,2,3,4-tetrahydronaphthalen-1-yl)phenyl)-1H- pyrazol-1-yl)azetidin-1-yl)-2-oxoethyl)-6,7- dihydropyrrolo[3,4-f]isoindole-1,3(2H,5H)-dione |
| B60 | 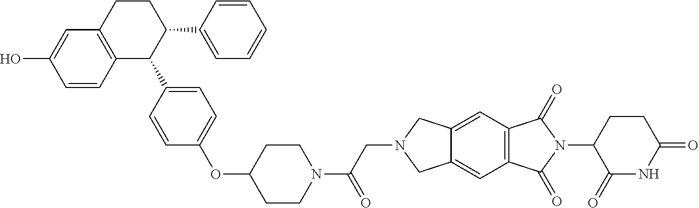 [SMILES: O=C1CCC(N2C(=O)c3cc4c(cc3C2=O)CN(CC(=O)N2CCC(Oc3ccc([C@@H]5c6ccc(O)cc6CC[C@@H]5c5ccccc5)cc3)CC2)C4)C(=O)N1] | 2-(2,6-dioxopiperidin-3-yl)-6-(2-(4-(4-((1R,2S)-6-hydroxy-2- phenyl-1,2,3,4-tetrahydronaphthalen-1-yl)phenoxy)piperidin-1- yl)-2-oxoethyl)-6,7-dihydropyrrolo[3,4-f]isoindole- 1,3(2H,5H)-dione |
| B61 | 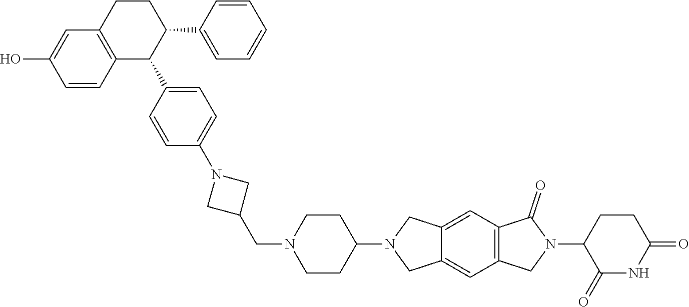 [SMILES: O=C1CCC(N2Cc3cc4c(cc3C2=O)CN(C2CCN(CC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(1-((1-(4-((1R,2S)-6-hydroxy-2- phenyl-1,2,3,4-tetrahydronaphthalen-1-yl)phenyl)azetidin-3- yl)methyl)piperidin-4-yl)-6,7-dihydropyrrolo[3,4-f]isoindole- 1,3(2H,5H)-dione |
| B62 | 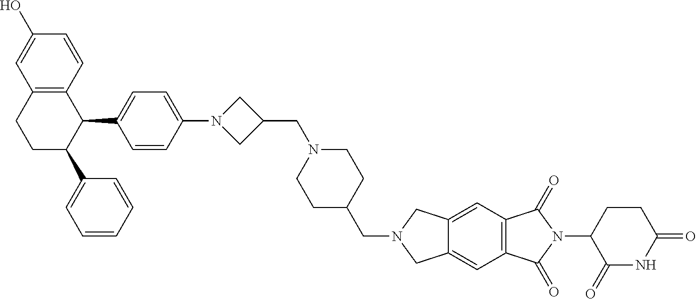 [SMILES: O=C1CCC(N2C(=O)c3cc4c(cc3C2=O)CN(CC2CCN(CC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-((1-(4-((1R,2S)-6-hydroxy-2- phenyl-1,2,3,4-tetrahydronaphthalen-1-yl)phenyl)azetidin-3- yl)methyl)piperidin-4-yl)methyl)-6,7-dihydropyrrolo[3,4- f]isoindole-1,3(2H,5H)-dione |
| B63 | 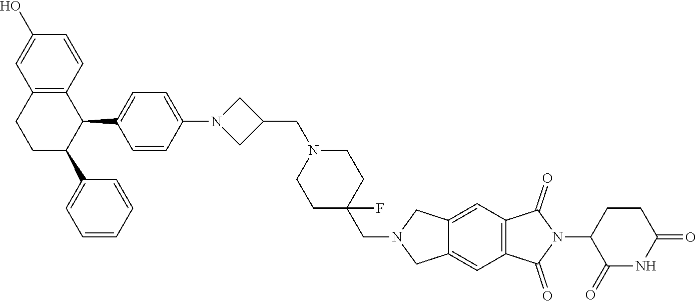 [SMILES: O=C1CCC(N2C(=O)c3cc4c(cc3C2=O)CN(CC2(F)CCN(CC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((4-fluoro-1-((1-(4-((1R,2S)-6- hydroxy-2-phenyl-1,2,3,4-tetrahydronaphthalen-1- yl)phenyl)azetidin-3-yl)methyl)piperidin-4-yl)methyl)-6,7- dihydropyrrolo[3,4-f]isoindole-1,3(2H,5H)-dione |
| B64 | 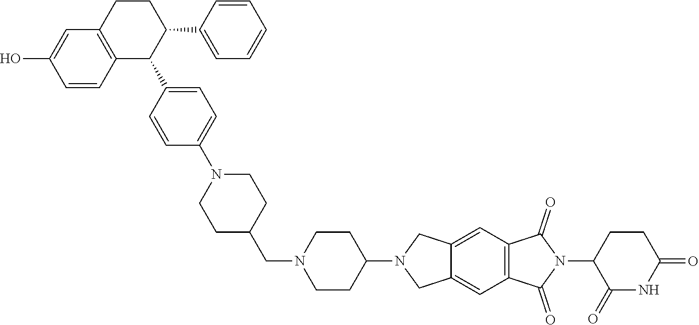 [SMILES: O=C1CCC(N2C(=O)c3cc4c(cc3C2=O)CN(C2CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-((1-(4-((1R,2S)-6-hydroxy-2- phenyl-1,2,3,4-tctrahydronaphthalcn-1-yl)phenyl)pipcridin-4- yl)methyl)piperidin-4-yl)-6,7-dihydropyrrolo[3,4-f]isoindole- 1,3(2H,5H)-dione |
| B65 | 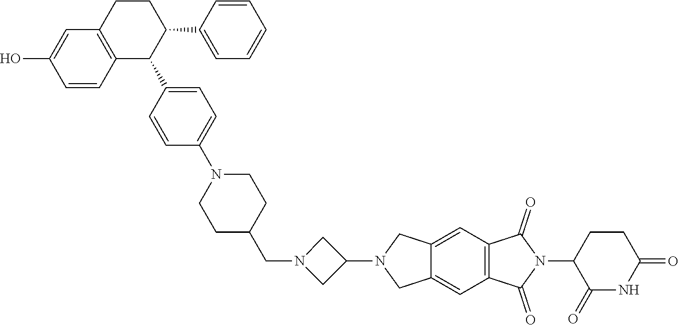 [SMILES: O=C1CCC(N2C(=O)c3cc4c(cc3C2=O)CN(C2CN(CC3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(1-((1-(4-((1R,2S)-6-hydroxy-2- phenyl-1,2,3,4-tetrahydronaphthalen-1-yl)phenyl)piperidin-4- yl)methyl)azetidin-3-yl)-6,7-dihydropyrrolo[3,4-f]isoindole- 1,3(2H,5H)-dione |
| B66 | 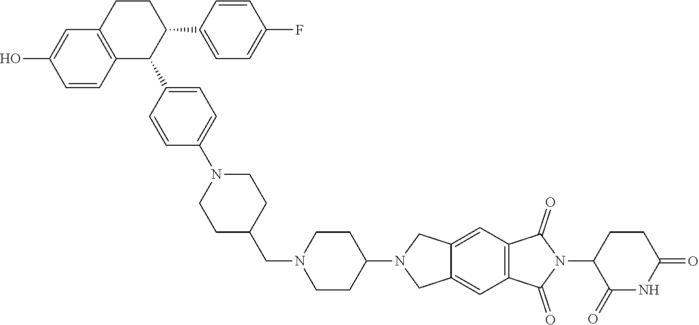 [SMILES: O=C1CCC(N2C(=O)c3cc4c(cc3C2=O)CN(C2CCN(CC3CCN(c5ccc([C@@H]6c7ccc(O)cc7CC[C@@H]6c6ccc(F)cc6)cc5)CC3)CC2)C4)C(=O)N1] | 2-(2,6-dioxopiperidin-3-yl)-6-(1-((1-(4-((1R,2S)-2-(4- fluorophenyl)-6-hydroxy-1,2,3,4-tetrahydronaphthalen-1- yl)phenyl)piperidin-4-yl)methyl)piperidin-4-yl)-6,7- dihydropyrrolo[3,4-f]isoindole-1,3(2H,5H)-dione |
| B67 | 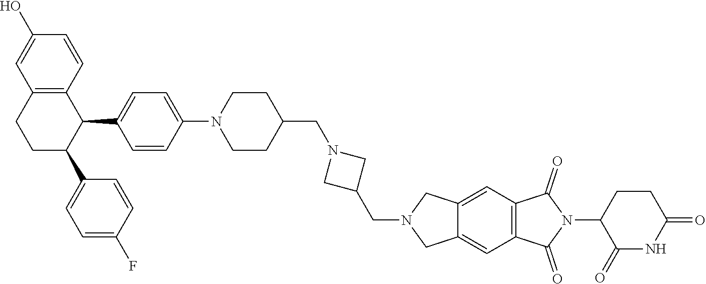 [SMILES: O=C1CCC(N2C(=O)c3cc4c(cc3C2=O)CN(CC2CN(CC3CCN(c5ccc([C@@H]6c7ccc(O)cc7CC[C@@H]6c6ccc(F)cc6)cc5)CC3)C2)C4)C(=O)N1] | 2-(2,6-dioxopiperidin-3-yl)-6-((1-((1-(4-((1R,2S)-2-(4- fluorophenyl)-6-hydroxy-1,2,3,4-tetrahydronaphthalen-1- yl)phenyl)piperidin-4-yl)methyl)azetidin-3-yl)methyl)-6,7- dihydropyrrolo[3,4-f]isoindole-1,3(2H,5H)-dione |
| B68 | 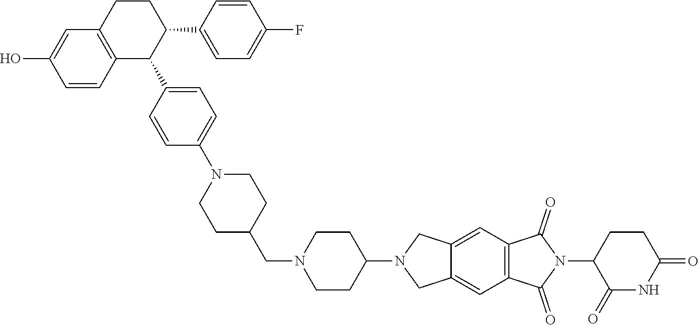 [SMILES: O=C1CCC(N2C(=O)c3cc4c(cc3C2=O)CN(C2CCN(CC3CCN(c5ccc([C@@H]6c7ccc(O)cc7CC[C@@H]6c6ccc(F)cc6)cc5)CC3)CC2)C4)C(=O)N1] | 3-(6-(1-((1-(4-((1R,2S)-2-(4-fluorophenyl)-6-hydroxy-1,2,3,4- tetrahydronaphthalen-1-yl)phenyl)piperidin-4- yl)methyl)piperidin-4-yl)-1-oxo-3,5,6,7-tetrahydropyrrolo[3,4- f]isoindol-2(1H)-yl)piperidine-2,6-dione |
| B69 | 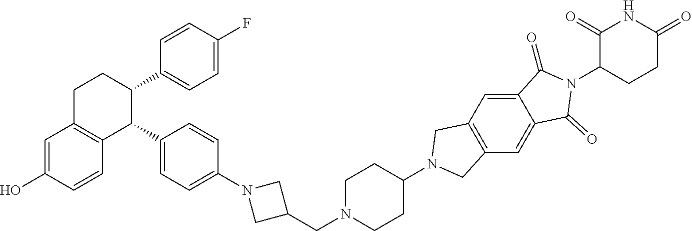 [SMILES: O=C1CCC(N2C(=O)c3cc4c(cc3C2=O)CN(C2CCN(CC3CN(c5ccc([C@@H]6c7ccc(O)cc7CC[C@@H]6c6ccc(F)cc6)cc5)C3)CC2)C4)C(=O)N1] | 2-(2,6-dioxopiperidin-3-yl)-6-(1-((1-(4-((1R,2S)-2-(4- fluorophenyl)-6-hydroxy-1,2,3,4-tetrahydronaphthalen-1- yl)phenyl)azetidin-3-yl)methyl)piperidin-4-yl)-6,7- dihydropyrrolo[3,4-f]isoindole-1,3(2H,5H)-dione |
| B70 | 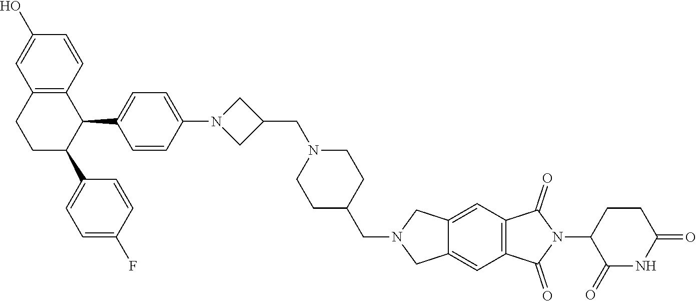 [SMILES: O=C1CCC(N2C(=O)c3cc4c(cc3C2=O)CN(CC2CCN(CC3CN(c5ccc([C@@H]6c7ccc(O)cc7CC[C@@H]6c6ccc(F)cc6)cc5)C3)CC2)C4)C(=O)N1] | 2-(2,6-dioxopiperidin-3-yl)-6-((1-((1-(4-((1R,2S)-2-(4- fluorophenyl)-6-hydroxy-1,2,3,4-tetrahydronaphthalen-1- yl)phenyl)azetidin-3-yl)methyl)piperidin-4-yl)methyl)-6,7- dihydropyrrolo[3,4-f]isoindole-1,3(2H,5H)-dione |
| B71 | 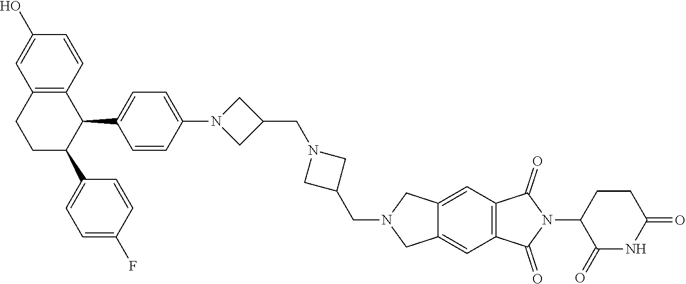 [SMILES: O=C1CCC(N2C(=O)c3cc4c(cc3C2=O)CN(CC2CN(CC3CN(c5ccc([C@@H]6c7ccc(O)cc7CC[C@@H]6c6ccc(F)cc6)cc5)C3)C2)C4)C(=O)N1] | 2-(2,6-dioxopiperidin-3-yl)-6-((1-((1-(4-((1R,2S)-2-(4- fluorophenyl)-6-hydroxy-1,2,3,4-tetrahydronaphthalen-1- yl)phenyl)azetidin-3-yl)methyl)azetidin-3-yl)methyl)-6,7- dihydropyrrolo[3,4-f]isoindole-1,3(2H,5H)-dione |
| B72 | 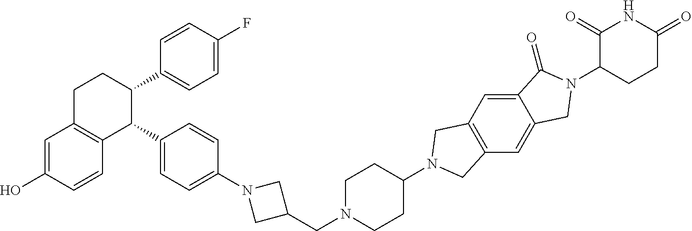 [SMILES: O=C1CCC(N2Cc3cc4c(cc3C2=O)CN(C2CCN(CC3CN(c5ccc([C@@H]6c7ccc(O)cc7CC[C@@H]6c6ccc(F)cc6)cc5)C3)CC2)C4)C(=O)N1] | 3-(6-(1-((1-(4-((1R,2S)-2-(4-fluorophenyl)-6-hydroxy-1,2,3,4- tetrahydronaphthalen-1-yl)phenyl)azetidin-3- yl)methyl)piperidin-4-yl)-1-oxo-3,5,6,7-tetrahydropyrrolo[3,4- f]isoindol-2(1H)-yl)pipcridinc-2,6-dionc |
| B73 | 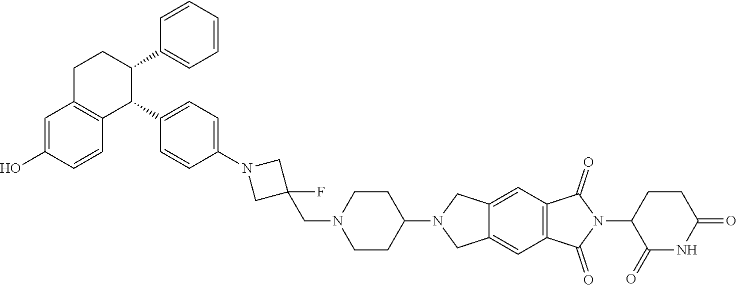 [SMILES: O=C1CCC(N2C(=O)c3cc4c(cc3C2=O)CN(C2CCN(CC3(F)CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(1-((3-fluoro-1-(4-((1R,2S)-6- hydroxy-2-phenyl-1,2,3,4-tetrahydronaphthalen-1- yl)phenyl)azetidin-3-yl)methyl)piperidin-4-yl)-6,7- dihydropyrrolo[3,4-f]isoindole-1,3(2H,5H)-dione |
| B74 | 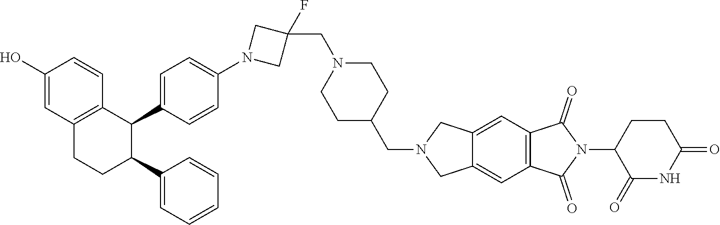 [SMILES: O=C1CCC(N2C(=O)c3cc4c(cc3C2=O)CN(CC2CCN(CC3(F)CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-((3-fluoro-1-(4-((1R,2S)-6- hydroxy-2-phenyl-1,2,3,4-tetrahydronaphthalen-1- yl)phenyl)azetidin-3-yl)methyl)piperidin-4-yl)methyl)-6,7- dihydropyrrolo[3,4-f]isoindole-1,3(2H,5H)-dione |
| B75 | 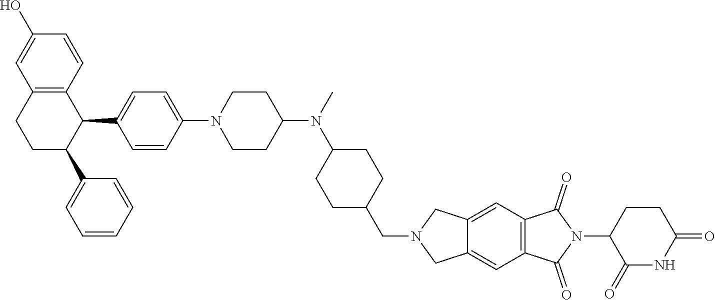 [SMILES: CN(C1CCC(CN2Cc3cc4c(cc3C2)C(=O)N(C2CCC(=O)NC2=O)C4=O)CC1)C1CCN(c2ccc([C@@H]3c4ccc(O)cc4CC[C@@H]3c3ccccc3)cc2)CC1] | 2-(2,6-dioxopiperidin-3-yl)-6-((4-((1-(4-((1R,2S)-6-hydroxy-2- phenyl-1,2,3,4-tetrahydronaphthalen-1-yl)phenyl)piperidin-4- yl)(methyl)amino)cyclohexyl)methyl)-6,7-dihydropyrrolo[3,4- f]isoindole-1,3(2H,5H)-dione |
| B76 | 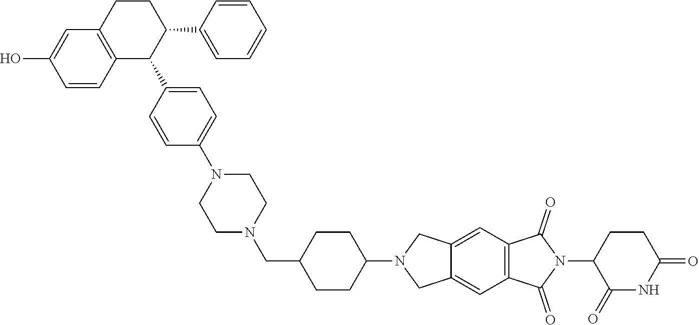 [SMILES: O=C1CCC(N2C(=O)c3cc4c(cc3C2=O)CN(C2CCC(CN3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(4-((4-(4-((1R,2S)-6-hydroxy-2- phenyl-1,2,3,4-tetrahydronaphthalen-1-yl)phenyl)piperazin-1- yl)methyl)cyclohexyl)-6,7-dihydropyrrolo[3,4-f]isoindole- 1,3(2H,5H)-dione |
| B77 | 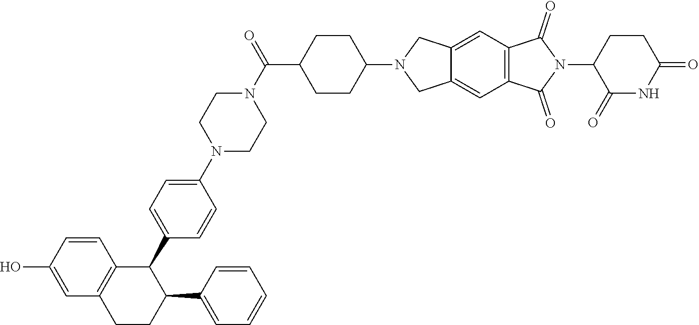 [SMILES: O=C1CCC(N2C(=O)c3cc4c(cc3C2=O)CN(C2CCC(C(=O)N3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(4-(4-(4-((1R,2S)-6-hydroxy-2- phenyl-1,2,3,4-tetrahydronaphthalen-1-yl)phenyl)piperazine-1- carbonyl)cyclohexyl)-6,7-dihydropyrrolo[3,4-f]isoindole- 1,3(2H,5H)-dione |
| B78 | 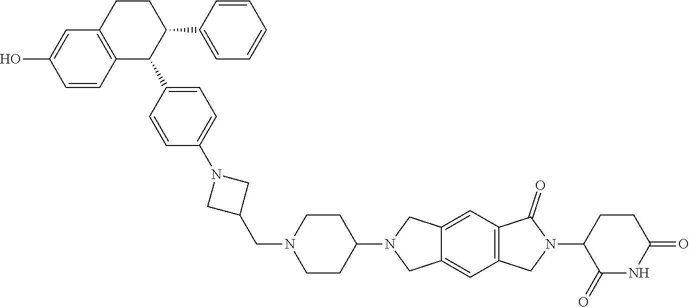 [SMILES: O=C1CCC(N2Cc3cc4c(cc3C2=O)CN(C2CCN(CC3CN(c5ccc([C@@H]6c7ccc(O)cc7CC[C@@H]6c6ccccc6)cc5)C3)CC2)C4)C(=O)N1] | 3-(6-(1-((1-(4-((1R,2S)-6-hydroxy-2-phenyl-1,2,3,4- tetrahydronaphthalen-1-yl)phenyl)azetidin-3- yl)methyl)piperidin-4-yl)-1-oxo-3,5,6,7-tetrahydropyrrolo[3,4- f]isoindol-2(1H)-yl)piperidine-2,6-dione |
| B79 | 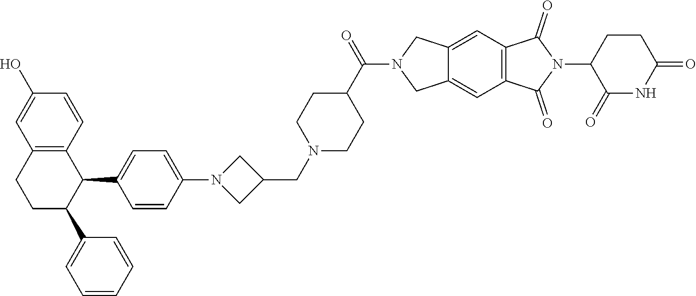 [SMILES: O=C1CCC(N2C(=O)c3cc4c(cc3C2=O)CN(C(=O)C2CCN(CC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(1-((1-(4-((1R,2S)-6-hydroxy-2- phenyl-1,2,3,4-tetrahydronaphthalen-1-yl)phenyl)azetidin-3- yl)methyl)piperidine-4-carbonyl)-6,7-dihydropyrrolo[3,4- f]isoindole-1,3(2H,5H)-dione |
| B80 | 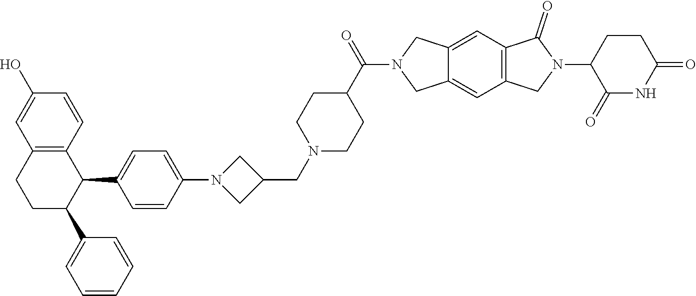 [SMILES: O=C1CCC(N2Cc3cc4c(cc3C2=O)CN(C(=O)C2CCN(CC3CN(c5ccc([C@@H]6c7ccc(O)cc7CC[C@@H]6c6ccccc6)cc5)C3)CC2)C4)C(=O)N1] | 3-(6-(1-((1-(4-((1R,2S)-6-hydroxy-2-phenyl-1,2,3,4- tetrahydronaphthalen-1-yl)phenyl)azetidin-3- yl)methyl)piperidine-4-carbonyl)-1-oxo-3,5,6,7- tetrahydropyrrolo[3,4-f]isoindol-2(1H)-yl)piperidine-2,6-dione |
| B81* | 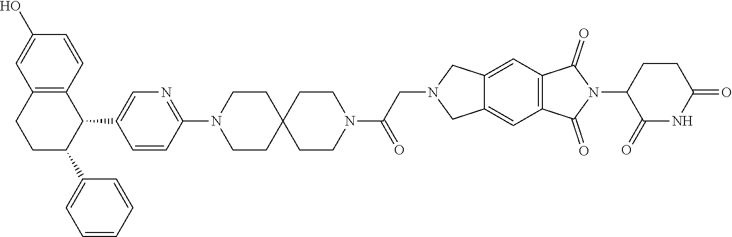 [SMILES: O=C1CCC(N2C(=O)c3cc4c(cc3C2=O)CN(CC(=O)N2CCC3(CC2)CCN(c2ccc([C@H]5c6ccc(O)cc6CC[C@H]5c5ccccc5)cn2)CC3)C4)C(=O)N1] | 2-(2,6-dioxopiperidin-3-yl)-6-(2-(9-(5-((1R,2R)-6-hydroxy-2- phenyl-1,2,3,4-tetrahydronaphthalen-1-yl)pyridin-2-yl)-3,9- diazaspiro[5.5]undecan-3-yl)-2-oxoethyl)-6,7- dihydropyrrolo[3,4-f]isoindole-1,3(2H,5H)-dione |
| B82* | 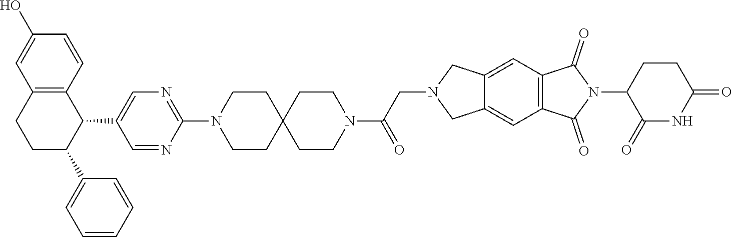 [SMILES: O=C1CCC(N2C(=O)c3cc4c(cc3C2=O)CN(CC(=O)N2CCC3(CC2)CCN(c2ncc([C@H]5c6ccc(O)cc6CC[C@H]5c5ccccc5)cn2)CC3)C4)C(=O)N1] | 2-(2,6-dioxopiperidin-3-yl)-6-(2-(9-(5-((1R,2R)-6-hydroxy-2- phenyl-1,2,3,4-tetrahydronaphthalen-1-yl)pyrimidin-2-yl)-3,9- diazaspiro[5.5]undecan-3-yl)-2-oxoethyl)-6,7- dihydropyrrolo[3,4-f]isoindole-1,3(2H,5H)-dione |
| B83 | 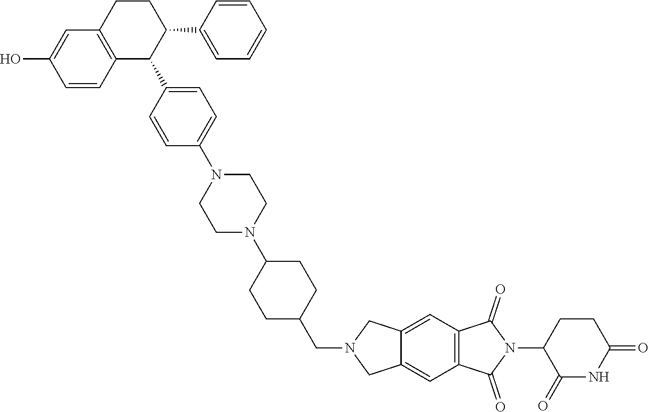 [SMILES: O=C1CCC(N2C(=O)c3cc4c(cc3C2=O)CN(CC2CCC(N3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((4-(4-(4-((1R,2S)-6-hydroxy-2- phenyl-1,2,3,4-tetrahydronaphthalen-1-yl)phenyl)piperazin-1- yl)cyclohcxyl)mcthyl)-6,7-dihydropyrrolo[3,4-f]isoindolc- 1,3(2H,5H)-dione |
| B84 | 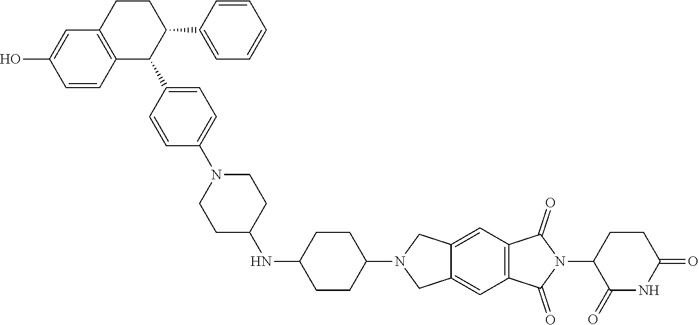 [SMILES: O=C1CCC(N2C(=O)c3cc4c(cc3C2=O)CN(C2CCC(NC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(4-((1-(4-((1R,2S)-6-hydroxy-2- phenyl-1,2,3,4-tetrahydronaphthalen-1-yl)phenyl)piperidin-4- yl)amino)cyclohexyl)-6,7-dihydropyrrolo[3,4-f]isoindole- 1,3(2H,5H)-dione |
| B85* | 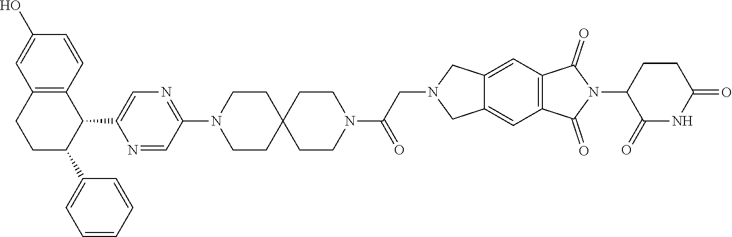 [SMILES: O=C1CCC(N2C(=O)c3cc4c(cc3C2=O)CN(CC(=O)N2CCC3(CC2)CCN(c2cnc([C@H]5c6ccc(O)cc6CC[C@H]5c5ccccc5)cn2)CC3)C4)C(=O)N1] | 2-(2,6-dioxopiperidin-3-yl)-6-(2-(9-(5-((1R,2R)-6-hydroxy-2- phenyl-1,2,3,4-tetrahydronaphthalen-1-yl)pyrazin-2-yl)-3,9- diazaspiro[5.5]undecan-3-yl)-2-oxoethyl)-6,7- dihydropyrrolo[3,4-f]isoindole-1,3(2H,5H)-dione |
| B86* | 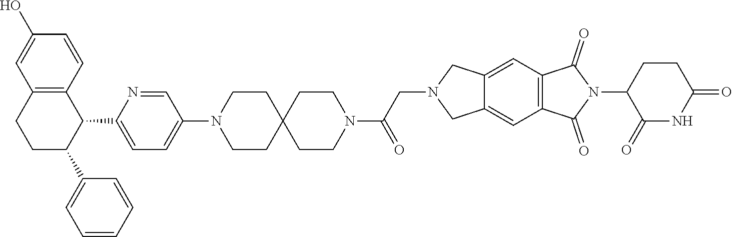 [SMILES: O=C1CCC(N2C(=O)c3cc4c(cc3C2=O)CN(CC(=O)N2CCC3(CC2)CCN(c2ccc([C@H]5c6ccc(O)cc6CC[C@H]5c5ccccc5)nc2)CC3)C4)C(=O)N1] | 2-(2,6-dioxopiperidin-3-yl)-6-(2-(9-(6-((1R,2R)-6-hydroxy-2- phenyl-1,2,3,4-tetrahydronaphthalen-1-yl)pyridin-3-yl)-3,9- diazaspiro[5.5]undecan-3-yl)-2-oxoethyl)-6,7- dihydropyrrolo[3,4-f]isoindole-1,3(2H,5H)-dione |
| B87* | 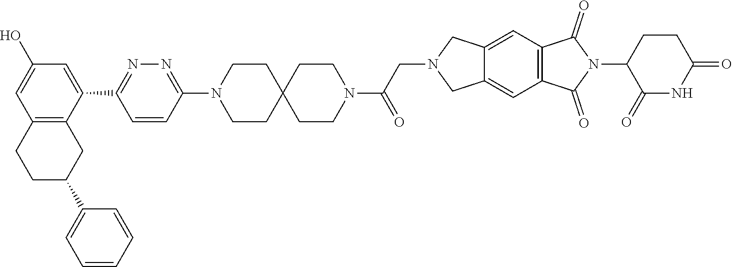 | 2-(2,6-dioxopiperidin-3-yl)-6-(2-(9-(6-((1R,2R)-6-hydroxy-2- phenyl-1,2,3,4-tetrahydronaphthalen-1-yl)pyridazin-3-yl)-3,9- diazaspiro[5.5]undecan-3-yl)-2-oxoethyl)-6,7- dihydropyrrolo[3,4-f]isoindole-1,3(2H,5H)-dione |
| B88 | 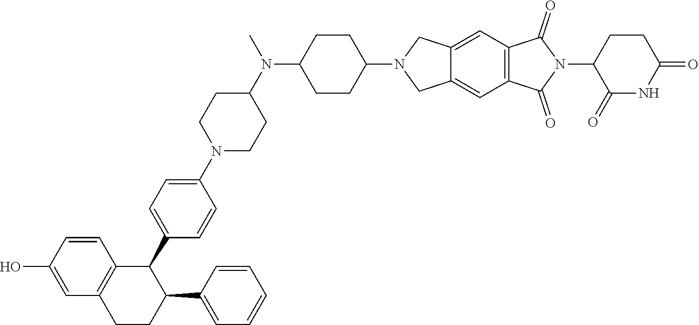 [SMILES: CN(C1CCC(N2Cc3cc4c(cc3C2)C(=O)N(C2CCC(=O)NC2=O)C4=O)CC1)C1CCN(c2ccc([C@@H]3c4ccc(O)cc4CC[C@@H]3c3ccccc3)cc2)CC1] | 2-(2,6-dioxopiperidin-3-yl)-6-(4-((1-(4-((1R,2S)-6-hydroxy-2- phenyl-1,2,3,4-tetrahydronaphthalen-1-yl)phenyl)piperidin-4- yl)(methyl)amino)cyclohexyl)-6,7-dihydropyrrolo[3,4- f]isoindole-1,3(2H,5H)-dione |
| B89 | 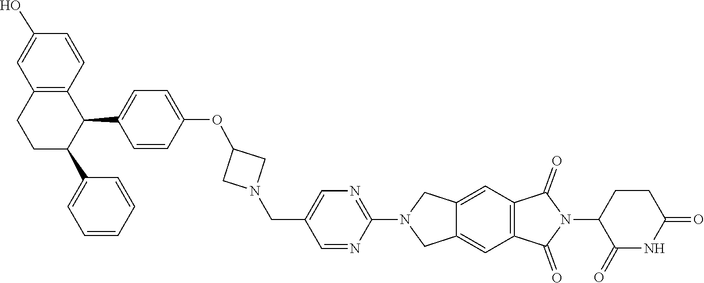 [SMILES: O=C1CCC(N2C(=O)c3cc4c(cc3C2=O)CN(c2ncc(CN3CC(Oc5ccc([C@@H]6c7ccc(O)cc7CC[C@@H]6c6ccccc6)cc5)C3)cn2)C4)C(=O)N1] | 2-(2,6-dioxopiperidin-3-yl)-6-(5-((3-(4-((1R,2S)-6-hydroxy-2- phenyl-1,2,3,4-tetrahydronaphthalen-1-yl)phenoxy)azetidin-1- yl)methyl)pyrimidin-2-yl)-6,7-dihydropyrrolo[3,4-f]isoindole- 1,3(2H,5H)-dione |
| B90 | 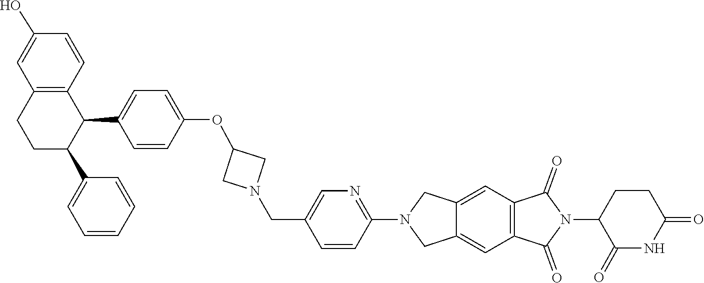 [SMILES: O=C1CCC(N2C(=O)c3cc4c(cc3C2=O)CN(c2ccc(CN3CC(Oc5ccc([C@@H]6c7ccc(O)cc7CC[C@@H]6c6ccccc6)cc5)C3)cn2)C4)C(=O)N1] | 2-(2,6-dioxopiperidin-3-yl)-6-(5-((3-(4-((1R,2S)-6-hydroxy-2- phenyl-1,2,3,4-tetrahydronaphthalen-1-yl)phenoxy)azetidin-1- yl)methyl)pyridin-2-yl)-6,7-dihydropyrrolo[3,4-f]isoindole- 1,3(2H,5H)-dione |
| B91 | 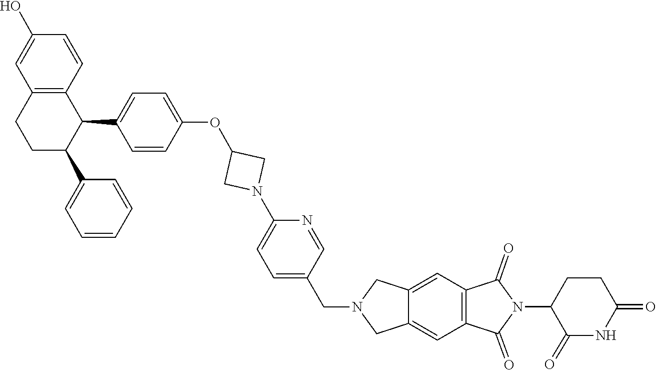 [SMILES: O=C1CCC(N2C(=O)c3cc4c(cc3C2=O)CN(Cc2ccc(N3CC(Oc5ccc([C@@H]6c7ccc(O)cc7CC[C@@H]6c6ccccc6)cc5)C3)nc2)C4)C(=O)N1] | 2-(2,6-dioxopiperidin-3-yl)-6-((6-(3-(4-((1R,2S)-6-hydroxy-2- phenyl-1,2,3,4-tetrahydronaphthalen-1-yl)phenoxy)azetidin-1- yl)pyridin-3-yl)methyl)-6,7-dihydropyrrolo[3,4-f]isoindole- 1,3(2H,5H)-dione |
| B92 | 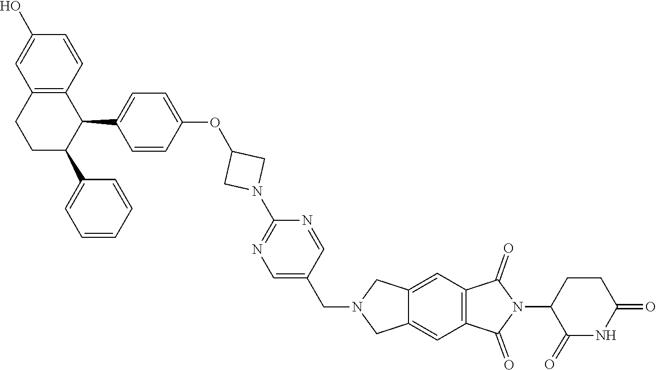 [SMILES: O=C1CCC(N2C(=O)c3cc4c(cc3C2=O)CN(Cc2cnc(N3CC(Oc5ccc([C@@H]6c7ccc(O)cc7CC[C@@H]6c6ccccc6)cc5)C3)nc2)C4)C(=O)N1] | 2-(2,6-dioxopiperidin-3-yl)-6-((2-(3-(4-((1R,2S)-6-hydroxy-2- phenyl-1,2,3,4-tetrahydronaphthalen-1-yl)phenoxy)azetidin-1- yl)pyrimidin-5-yl)methyl)-6,7-dihydropyrrolo[3,4-f]isoindole- 1,3(2H,5H)-dione |
| B93 | 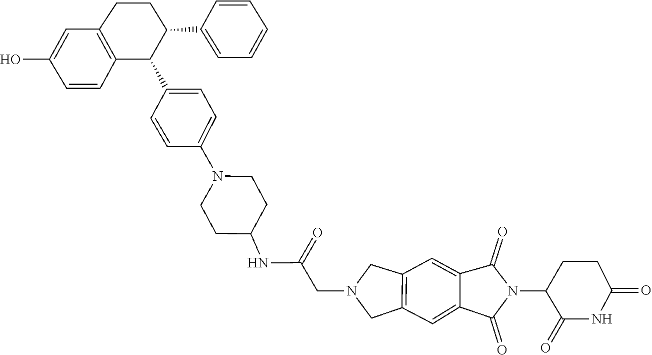 [SMILES: O=C1CCC(N2C(=O)c3cc4c(cc3C2=O)CN(CC(=O)NC2CCN(c3ccc([C@@H]5c6ccc(O)cc6CC[C@@H]5c5ccccc5)cc3)CC2)C4)C(=O)N1] | 2-(6-(2,6-dioxopiperidin-3-yl)-5,7-dioxo-3,5,6,7- tetrahydropyrrolo[3,4-f]isoindol-2(1H)-yl)-N-(1-(4-((1R,2S)-6- hydroxy-2-phenyl-1,2,3,4-tetrahydronaphthalen-1- yl)phenyl)piperidin-4-yl)acetamide |
| B94 | 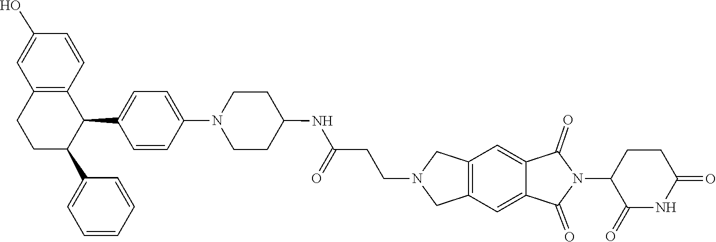 [SMILES: O=C1CCC(N2C(=O)c3cc4c(cc3C2=O)CN(CCC(=O)NC2CCN(c3ccc([C@@H]5c6ccc(O)cc6CC[C@@H]5c5ccccc5)cc3)CC2)C4)C(=O)N1] | 3-(6-(2,6-dioxopiperidin-3-yl)-5,7-dioxo-3,5,6,7- tetrahydropyrrolo[3,4-f]isoindol-2(1H)-yl)-N-(1-(4-((1R,2S)-6- hydroxy-2-phenyl-1,2,3,4-tetrahydronaphthalen-1- yl)phenyl)piperidin-4-yl)propenamide |
| B95 | 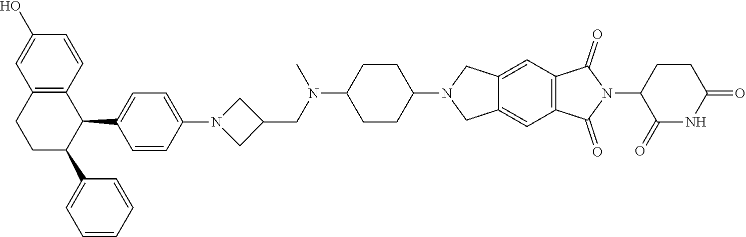 [SMILES: CN(CC1CN(c2ccc([C@@H]3c4ccc(O)cc4CC[C@@H]3c3ccccc3)cc2)C1)C1CCC(N2Cc3cc4c(cc3C2)C(=O)N(C2CCC(=O)NC2=O)C4=O)CC1] | 2-(2,6-dioxopiperidin-3-yl)-6-(4-(((1-(4-((1R,2S)-6-hydroxy-2- phenyl-1,2,3,4-tetrahydronaphthalen-1-yl)phenyl)azetidin-3- yl)methyl)(methyl)amino)cyclohexyl)-6,7-dihydropyrrolo[3,4- f]isoindole-1,3(2H,5H)-dione |
| B96 | 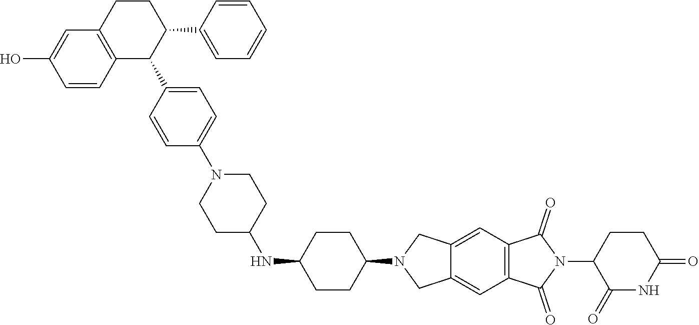 [SMILES: O=C1CCC(N2C(=O)c3cc4c(cc3C2=O)CN([C@H]2CC[C@@H](NC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1S,4s)-4-((1-(4-((1R,2S)-6- hydroxy-2-phenyl-1,2,3,4-tetrahydronaphthalen-1- yl)phenyl)piperidin-4-yl)amino)cyclohexyl)-6,7- dihydropyrrolo[3,4-f]isoindole-1,3(2H,5H)-dione |
| B97 | 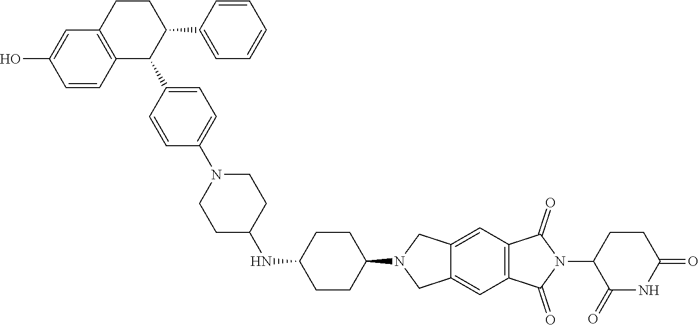 [SMILES: O=C1CCC(N2C(=O)c3cc4c(cc3C2=O)CN([C@H]2CC[C@H](NC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1R,4r)-4-((1-(4-((1R,2S)-6- hydroxy-2-phenyl-1,2,3,4-tetrahydronaphthalen-1- yl)phenyl)piperidin-4-yl)amino)cyclohexyl)-6,7- dihydropyrrolo[3,4-f]isoindole-1,3(2H,5H)-dione |
| B98 | 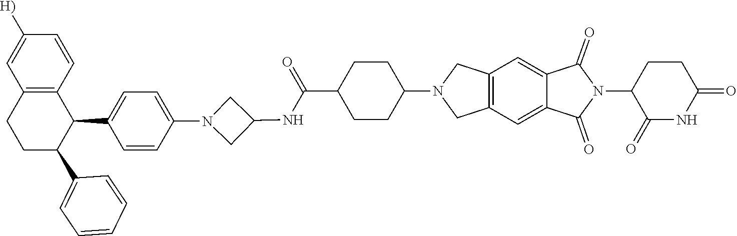 | 4-(6-(2,6-dioxopiperidin-3-yl)-5,7-dioxo-3,5,6,7- tetrahydropyrrolo[3,4-f]isoindol-2(1H)-yl)-N-(1-(4-((1R,2S)-6- hydroxy-2-phenyl-1,2,3,4-tetrahydronaphthalen-1- yl)phenyl)azetidin-3-yl)cyclohexane-1-carboxamide |
| B99 | 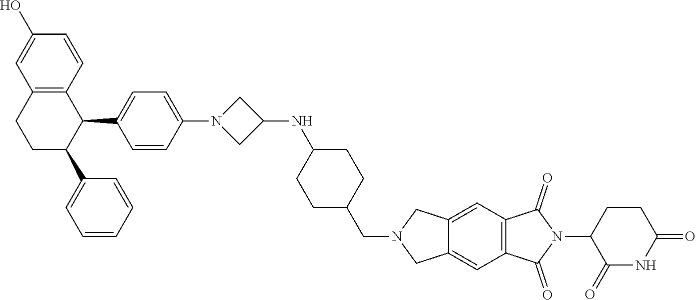 [SMILES: O=C1CCC(N2C(=O)c3cc4c(cc3C2=O)CN(CC2CCC(NC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((4-((1-(4-((1R,2S)-6-hydroxy-2- phenyl-1,2,3,4-tetrahydronaphthalen-1-yl)phenyl)azetidin-3- yl)amino)cyclohexyl)methyl)-6,7-dihydropyrrolo[3,4- f]isoindole-1,3(2H,5H)-dione |
| B100 | 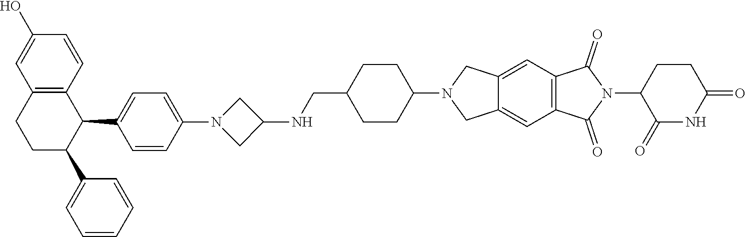 [SMILES: O=C1CCC(N2C(=O)c3cc4c(cc3C2=O)CN(C2CCC(CNC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(4-(((1-(4-((1R,2S)-6-hydroxy-2- phenyl-1,2,3,4-tetrahydronaphthalen-1-yl)phenyl)azetidin-3- yl)amino)methyl)cyclohexyl)-6,7-dihydropyrrolo[3,4- f]isoindole-1,3(2H,5H)-dione |
| B101 | 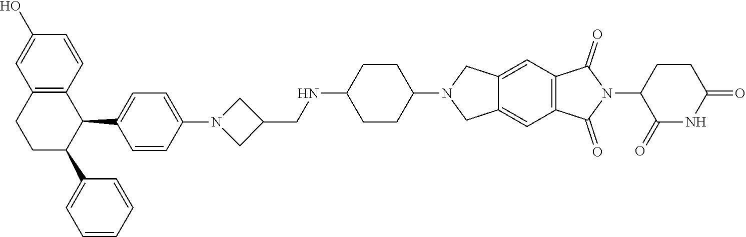 [SMILES: O=C1CCC(N2C(=O)c3cc4c(cc3C2=O)CN(C2CCC(NCC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(4-(((1-(4-((1R,2S)-6-hydroxy-2- phenyl-1,2,3,4-tetrahydronaphthalen-1-yl)phenyl)azetidin-3- yl)methyl)amino)cyclohexyl)-6,7-dihydropyrrolo[3,4- f]isoindole-1,3(2H,5H)-dione |
| B102 | 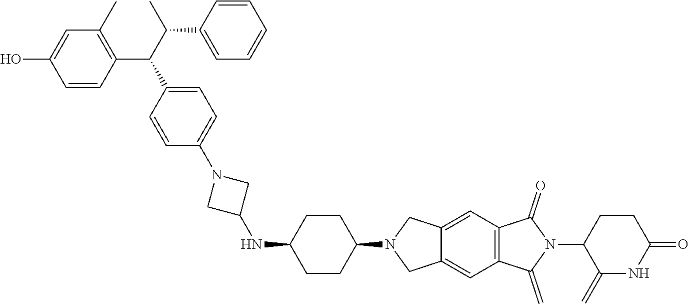 [SMILES: C=C1NC(=O)CCC1N1C(=C)c2cc3c(cc2C1=O)CN([C@H]1CC[C@@H](NC2CN(c4ccc([C@H](c5ccc(O)cc5C)[C@H](C)c5ccccc5)cc4)C2)CC1)C3] | 2-(2,6-dioxopiperidin-3-yl)-6-((1S,4s)-4-((1-(4-((1R,2S)-6- hydroxy-2-phenyl-1,2,3,4-tetrahydronaphthalen-1- yl)phenyl)azetidin-3-yl)amino)cyclohexyl)-6,7- dihydropyrrolo[3,4-f]isoindole-1,3(2H,5H)-dione |
| B103 | 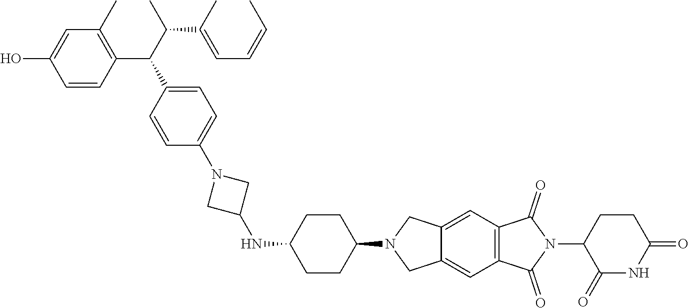 [SMILES: C/C=C\C=C(/C)[C@@H](C)[C@H](c1ccc(N2CC(N[C@H]3CC[C@H](N4Cc5cc6c(cc5C4)C(=O)N(C4CCC(=O)NC4=O)C6=O)CC3)C2)cc1)c1ccc(O)cc1C] | 2-(2,6-dioxopiperidin-3-yl)-6-((1R,4r)-4-((1-(4-((1R,2S)-6- hydroxy-2-phenyl-1,2,3,4-tctrahydronaphthalen-1- yl)phenyl)azetidin-3-yl)amino)cyclohexyl)-6,7- dihydropyrrolo[3,4-f]isoindole-1,3(2H,5H)-dione |
| B104 | 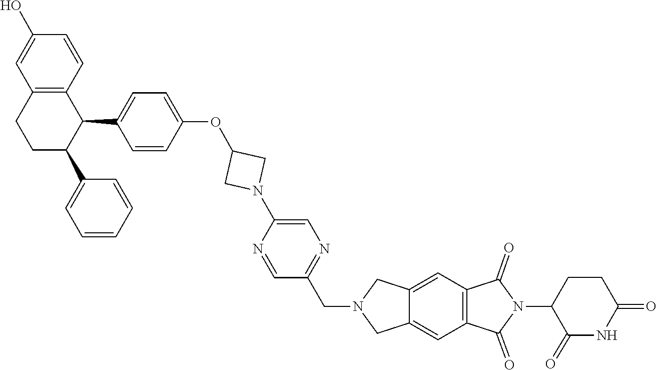 [SMILES: O=C1CCC(N2C(=O)c3cc4c(cc3C2=O)CN(Cc2cnc(N3CC(Oc5ccc([C@@H]6c7ccc(O)cc7CC[C@@H]6c6ccccc6)cc5)C3)cn2)C4)C(=O)N1] | 2-(2,6-dioxopiperidin-3-yl)-6-((5-(3-(4-((1R,2S)-6-hydroxy-2- phenyl-1,2,3,4-tetrahydronaphthalen-1-yl)phenoxy)azetidin-1- yl)pyrazin-2-yl)methyl)-6,7-dihydropyrrolo[3,4-f]isoindole- 1,3(2H,5H)-dione |
| B105 | 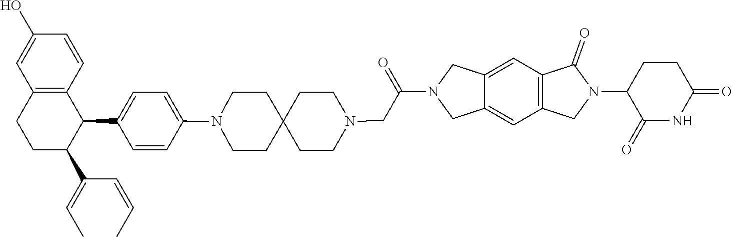 [SMILES: C/C=C\C(=C/C)[C@H]1CCc2cc(O)ccc2[C@H]1c1ccc(N2CCC3(CCN(CC(=O)N4Cc5cc6c(cc5C4)C(=O)N(C4CCC(=O)NC4=O)C6)CC3)CC2)cc1] | 3-(6-(2-(9-(4-((1R,2S)-6-hydroxy-2-phenyl-1,2,3,4- tetrahydronaphthalen-1-yl)phenyl)-3,9-diazaspiro[5.5]undecan- 3-yl)acetyl)-1-oxo-3,5,6,7-tetrahydropyrrolo[3,4-f]isoindol- 2(1H)-yl)piperidine-2,6-dione |
| B106 | 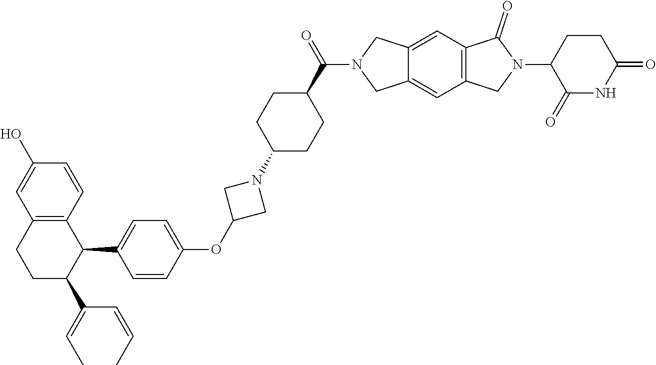 [SMILES: C/C=C\C(=C/C)[C@H]1CCc2cc(O)ccc2[C@H]1c1ccc(OC2CN([C@H]3CC[C@H](C(=O)N4Cc5cc6c(cc5C4)C(=O)N(C4CCC(=O)NC4=O)C6)CC3)C2)cc1] | 3-(6-((1R,4r)-4-(3-(4-((1R,2S)-6-hydroxy-2-phenyl-1,2,3,4- tetrahydronaphthalen-1-yl)phenoxy)azetidin-1-yl)cyclohexane- 1-carbonyl)-1-oxo-3,5,6,7-tetrahydropyrrolo[3,4-f]isoindol- 2(1H)-yl)piperidine-2,6-dione |
| B107 | 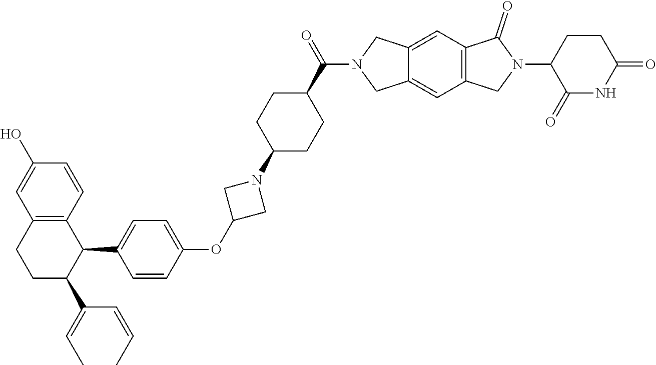 [SMILES: C/C=C\C(=C/C)[C@H]1CCc2cc(O)ccc2[C@H]1c1ccc(OC2CN([C@H]3CC[C@@H](C(=O)N4Cc5cc6c(cc5C4)C(=O)N(C4CCC(=O)NC4=O)C6)CC3)C2)cc1] | 3-(6-((1S,4s)-4-(3-(4-((1R,2S)-6-hydroxy-2-phenyl-1,2,3,4- tetrahydronaphthalen-1-yl)phenoxy)azetidin-1-yl)cyclohexane- 1-carbonyl)-1-oxo-3,5,6,7-tetrahydropyrrolo[3,4-f]isoindol- 2(1H)-yl)piperidine-2,6-dione |
| B108 | 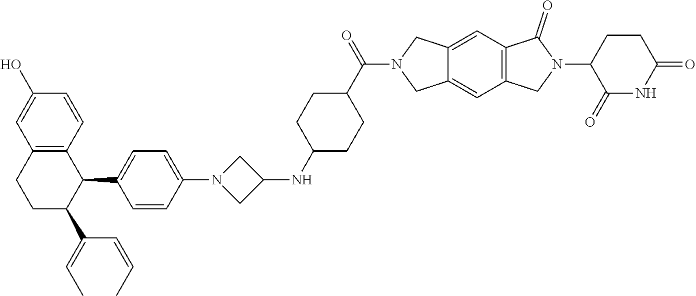 [SMILES: C/C=C\C(=C/C)[C@H]1CCc2cc(O)ccc2[C@H]1c1ccc(N2CC(NC3CCC(C(=O)N4Cc5cc6c(cc5C4)C(=O)N(C4CCC(=O)NC4=O)C6)CC3)C2)cc1] | 3-(6-(4-((1-(4-((1R,2S)-6-hydroxy-2-phenyl-1,2,3,4- tetrahydronaphthalen-1-yl)phenyl)azetidin-3- yl)amino)cyclohexane-1-carbonyl)-1-oxo-3,5,6,7- tetrahydropyrrolo[3,4-f]isoindol-2(1H)-yl)piperidine-2,6-dione |
| B109 | 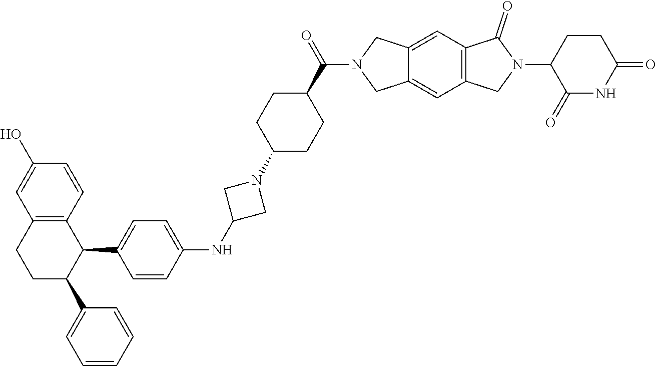 [SMILES: O=C1CCC(N2Cc3cc4c(cc3C2=O)CN(C(=O)[C@H]2CC[C@H](N3CC(Nc5ccc([C@@H]6c7ccc(O)cc7CC[C@@H]6c6ccccc6)cc5)C3)CC2)C4)C(=O)N1] | 3-(6-((1R,4r)-4-(3-((4-((1R,2S)-6-hydroxy-2-phenyl-1,2,3,4- tetrahydronaphthalen-1-yl)phenyl)amino)azetidin-1- yl)cyclohexane-1-carbonyl)-1-oxo-3,5,6,7- tetrahydropyrrolo[3,4-f]isoindol-2(1H)-yl)pipcridinc-2,6-dionc |
| B110 | 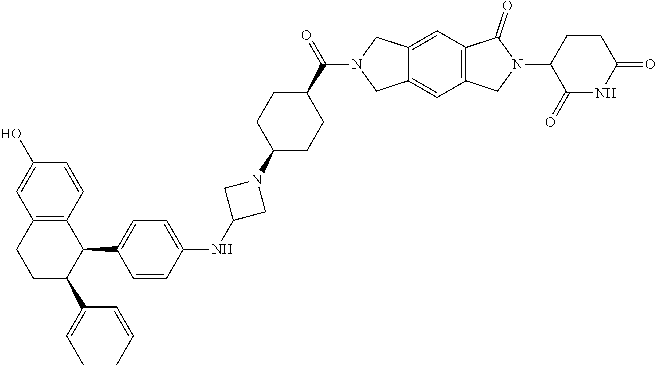 [SMILES: C/C=C\C(=C/C)[C@H]1CCc2cc(O)ccc2[C@H]1c1ccc(NC2CN([C@H]3CC[C@@H](C(=O)N4Cc5cc6c(cc5C4)C(=O)N(C4CCC(=O)NC4=O)C6)CC3)C2)cc1] | 3-(6-((1S,4s)-4-(3-((4-((1R,2S)-6-hydroxy-2-phenyl-1,2,3,4- tetrahydronaphthalcn-1-yl)phenyl)amino)azctidin-1- yl)cyclohexane-1-carbonyl)-1-oxo-3,5,6,7- tetrahydropyrrolo[3,4-f]isoindol-2(1H)-yl)piperidine-2,6-dione |
| B111 | 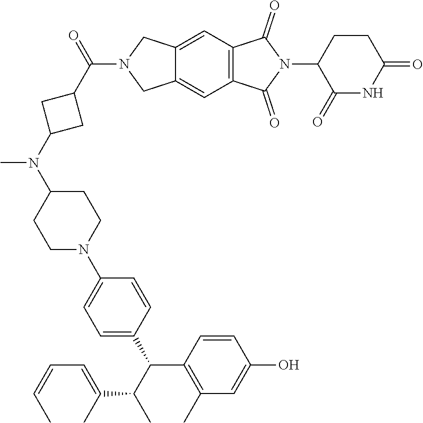 [SMILES: C/C=C\C=C(/C)[C@@H](C)[C@H](c1ccc(N2CCC(N(C)C3CC(C(=O)N4Cc5cc6c(cc5C4)C(=O)N(C4CCC(=O)NC4=O)C6=O)C3)CC2)cc1)c1ccc(O)cc1C] | 2-(2,6-dioxopiperidin-3-yl)-6-(3-((1-(4-((1R,2S)-6-hydroxy-2- phenyl-1,2,3,4-tetrahydronaphthalen-1-yl)phenyl)piperidin-4- yl)(methyl)amino)cyclobutane-1-carbonyl)-6,7- dihydropyrrolo[3,4-f]isoindole-1,3(2H,5H)-dione |
| B112 | 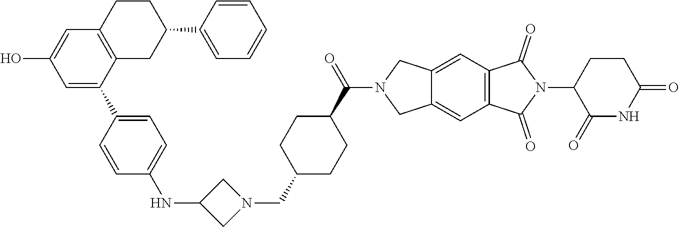 | 2-(2,6-dioxopiperidin-3-yl)-6-((1R,4r)-4-((3-((4-((1R,2S)-6- hydroxy-2-phenyl-1,2,3,4-tetrahydronaphthalen-1- yl)phenyl)amino)azetidin-1-yl)methyl)cyclohexane-1- carbonyl)-6,7-dihydropyrrolo[3,4-f]isoindole-1,3(2H,5H)- dione |
| B113 | 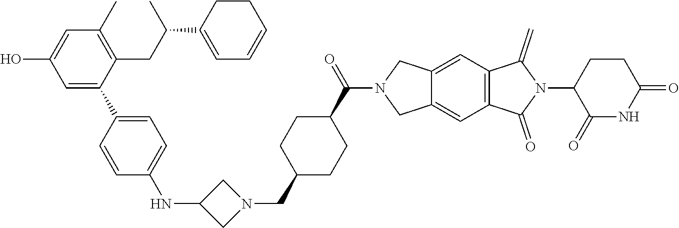 | 2-(2,6-dioxopiperidin-3-yl)-6-((1S,4s)-4-((3-((4-((1R,2S)-6- hydroxy-2-phenyl-1,2,3,4-tetrahydronaphthalen-1- yl)phenyl)amino)azetidin-1-yl)methyl)cyclohexane-1- carbonyl)-6,7-dihydropyrrolo[3,4-f]isoindole-1,3(2H,5H)- dionc |
| B114 | 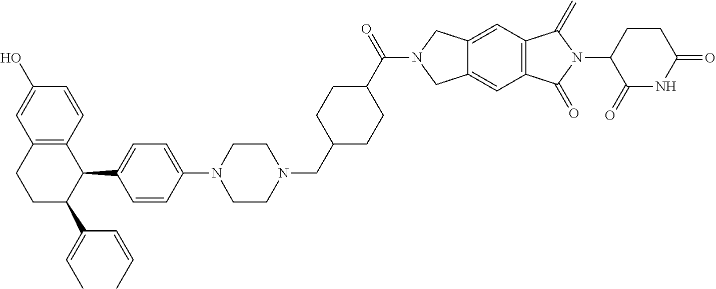 [SMILES: C=C1c2cc3c(cc2C(=O)N1C1CCC(=O)NC1=O)CN(C(=O)C1CCC(CN2CCN(c4ccc([C@@H]5c6ccc(O)cc6CC[C@@H]5C(/C=C\C)=C/C)cc4)CC2)CC1)C3] | 2-(2,6-dioxopiperidin-3-yl)-6-(4-((4-(4-((1R,2S)-6-hydroxy-2- phenyl-1,2,3,4-tetrahydronaphthalen-1-yl)phenyl)piperazin-1- yl)methyl)cyclohexane-1-carbonyl)-6,7-dihydropyrrolo[3,4- f]isoindole-1,3(2H,5H)-dione |
| B115 | 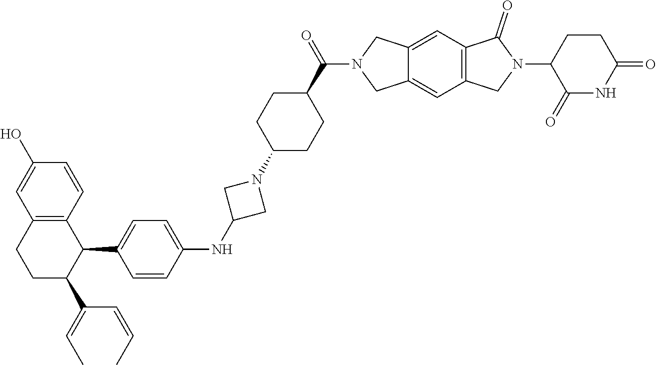 [SMILES: C/C=C\C(=C/C)[C@H]1CCc2cc(O)ccc2[C@H]1c1ccc(NC2CN([C@H]3CC[C@H](C(=O)N4Cc5cc6c(cc5C4)C(=O)N(C4CCC(=O)NC4=O)C6)CC3)C2)cc1] | 3-(6-((1R,4r)-4-(3-((4-((1R,2S)-6-hydroxy-2-phenyl-1,2,3,4- tetrahydronaphthalen-1-yl)phenyl)amino)azetidin-1- yl)cyclohexane-1-carbonyl)-1-oxo-3,5,6,7- tetrahydropyrrolo[3,4-f]isoindol-2(1H)-yl)piperidine-2,6-dione |
| B116 | 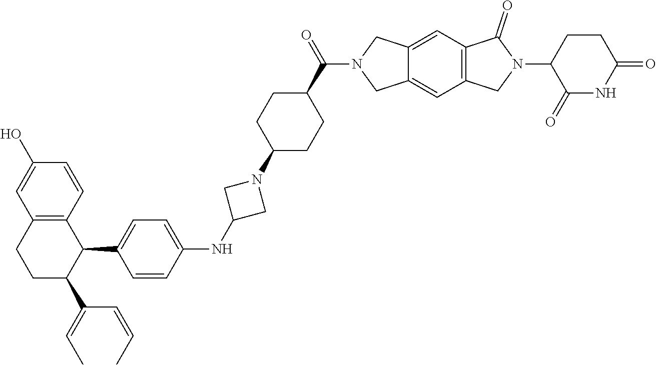 [SMILES: C/C=C\C(=C/C)[C@H]1CCc2cc(O)ccc2[C@H]1c1ccc(NC2CN([C@H]3CC[C@@H](C(=O)N4Cc5cc6c(cc5C4)C(=O)N(C4CCC(=O)NC4=O)C6)CC3)C2)cc1] | 3-(6-((1S,4s)-4-(3-((4-((1R,2S)-6-hydroxy-2-phenyl-1,2,3,4- tetrahydronaphthalen-1-yl)phenyl)amino)azetidin-1- yl)cyclohexane-1-carbonyl)-1-oxo-3,5,6,7- tetrahydropyrrolo[3,4-f]isoindol-2(1H)-yl)piperidine-2,6-dione |
| B117 | 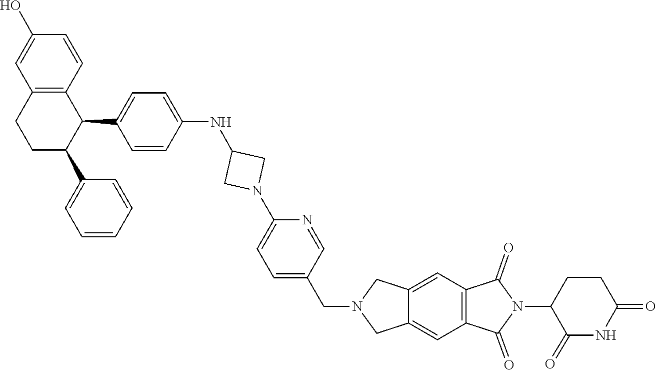 [SMILES: O=C1CCC(N2C(=O)c3cc4c(cc3C2=O)CN(Cc2ccc(N3CC(Nc5ccc([C@@H]6c7ccc(O)cc7CC[C@@H]6c6ccccc6)cc5)C3)nc2)C4)C(=O)N1] | 2-(2,6-dioxopiperidin-3-yl)-6-((6-(3-((4-((1R,2S)-6-hydroxy-2- phenyl-1,2,3,4-tetrahydronaphthalen-1- yl)phenyl)amino)azetidin-1-yl)pyridin-3-yl)methyl)-6,7- dihydropyrrolo[3,4-f]isoindole-1,3(2H,5H)-dione |
| B118 | 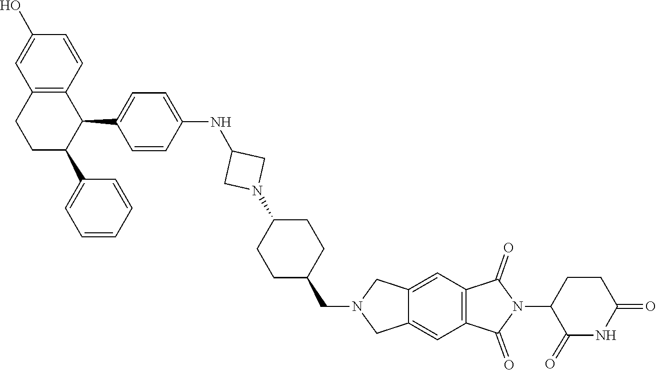 [SMILES: O=C1CCC(N2C(=O)c3cc4c(cc3C2=O)CN(C[C@H]2CC[C@H](N3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-(((1R,4r)-4-(3-((4-((1R,2S)-6- hydroxy-2-phenyl-1,2,3,4-tetrahydronaphthalen-1- yl)phenyl)amino)azetidin-1-yl)cyclohexyl)methyl)-6,7- dihydropyrrolo[3,4-f]isoindole-1,3(2H,5H)-dione |
| B119 | 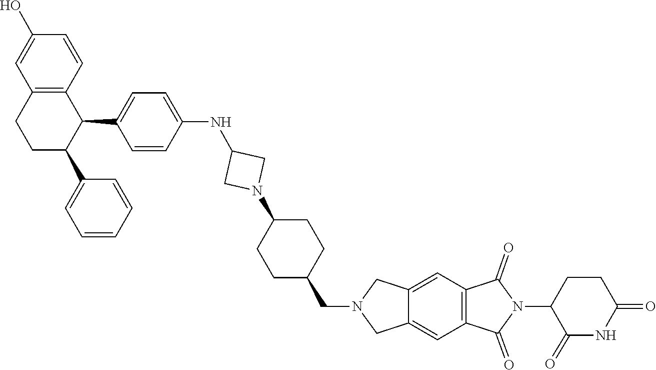 [SMILES: O=C1CCC(N2C(=O)c3cc4c(cc3C2=O)CN(C[C@H]2CC[C@@H](N3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-(((1S,4s)-4-(3-((4-((1R,2S)-6- hydroxy-2-phenyl-1,2,3,4-tetrahydronaphthalen-1- yl)phenyl)amino)azetidin-1-yl)cyclohexyl)methyl)-6,7- dihydropyrrolo[3,4-f]isoindole-1,3(2H,5H)-dione |
| B120 | 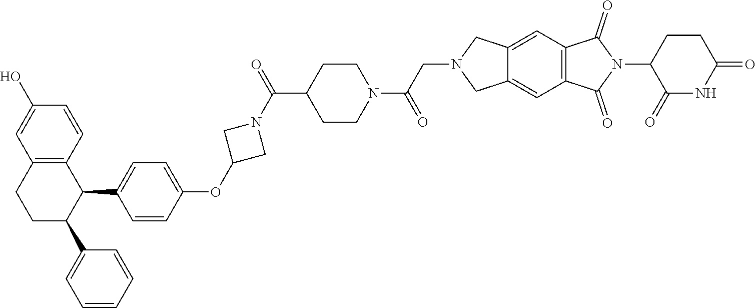 [SMILES: O=C1CCC(N2C(=O)c3cc4c(cc3C2=O)CN(CC(=O)N2CCC(C(=O)N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-(2-(4-(3-(4-((1R,2S)-6-hydroxy- 2-phenyl-1,2,3,4-tetrahydronaphthalen-1-yl)phenoxy)azetidine- 1-carbonyl)piperidin-1-yl)-2-oxoethyl)-6,7-dihydropyrrolo[3,4- f]isoindole-1,3(2H,5H)-dione |
| B121 | 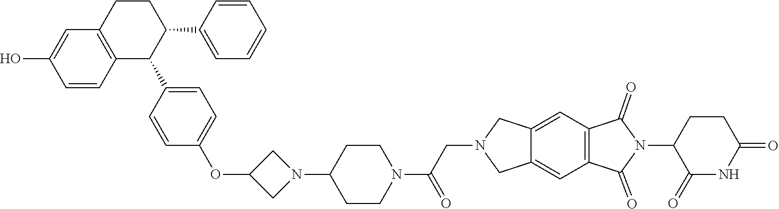 [SMILES: O=C1CCC(N2C(=O)c3cc4c(cc3C2=O)CN(CC(=O)N2CCC(N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-(2-(4-(3-(4-((1R,2S)-6-hydroxy- 2-phenyl-1,2,3,4-tetrahydronaphthalen-1-yl)phenoxy)azetidin- 1-yl)piperidin-1-yl)-2-oxoethyl)-6,7-dihydropyrrolo[3,4- f]isoindole-1,3(2H,5H)-dione |
| B122 | 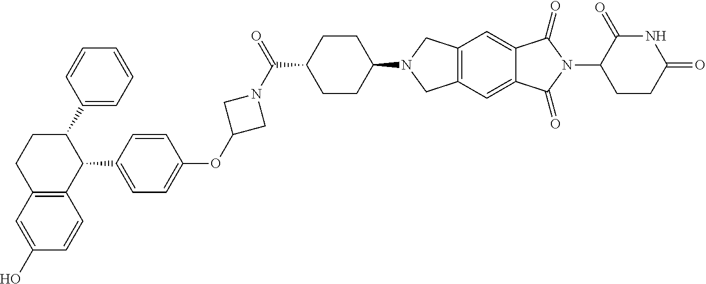 [SMILES: O=C1CCC(N2C(=O)c3cc4c(cc3C2=O)CN([C@H]2CC[C@H](C(=O)N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1S,4r)-4-(3-(4-((1R,2S)-6- hydroxy-2-phenyl-1,2,3,4-tctrahydronaphthalcn-1- yl)phenoxy)azetidine-1-carbonyl)cyclohexyl)-6,7- dihydropyrrolo[3,4-f]isoindole-1,3(2H,5H)-dione |
| B123 | 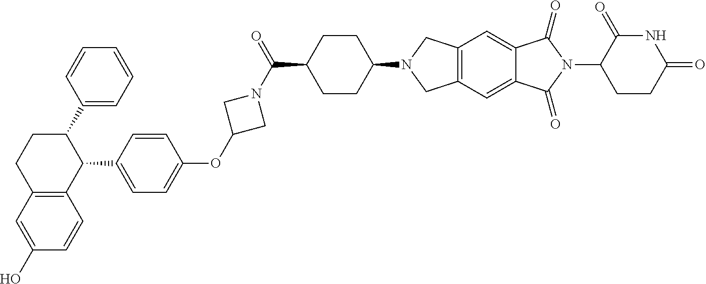 [SMILES: O=C1CCC(N2C(=O)c3cc4c(cc3C2=O)CN([C@H]2CC[C@@H](C(=O)N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1R,4s)-4-(3-(4-((1R,2S)-6- hydroxy-2-phenyl-1,2,3,4-tetrahydronaphthalen-1- yl)phenoxy)azetidine-1-carbonyl)cyclohexyl)-6,7- dihydropyrrolo[3,4-f]isoindole-1,3(2H,5H)-dione |
| B124 | 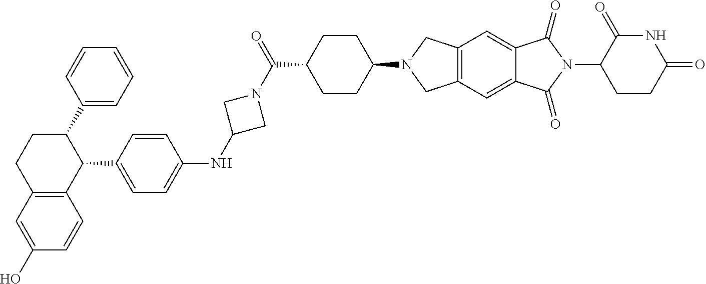 [SMILES: O=C1CCC(N2C(=O)c3cc4c(cc3C2=O)CN([C@H]2CC[C@H](C(=O)N3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1S,4r)-4-(3-((4-((1R,2S)-6- hydroxy-2-phenyl-1,2,3,4-tetrahydronaphthalen-1- yl)phenyl)amino)azetidine-1-carbonyl)cyclohexyl)-6,7- dihydropyrrolo[3,4-f]isoindole-1,3(2H,5H)-dione |
| B125 | 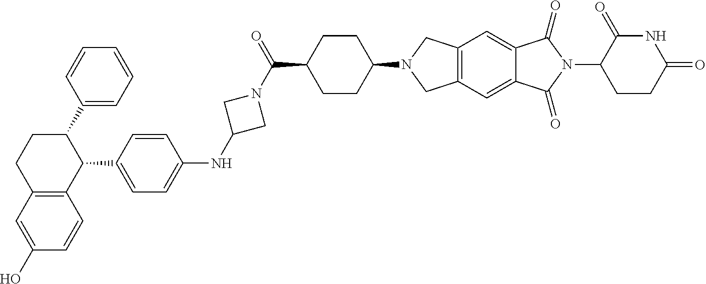 [SMILES: O=C1CCC(N2C(=O)c3cc4c(cc3C2=O)CN([C@H]2CC[C@@H](C(=O)N3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1R,4s)-4-(3-((4-((1R,2S)-6- hydroxy-2-phenyl-1,2,3,4-tetrahydronaphthalen-1- yl)phenyl)amino)azetidine-1-carbonyl)cyclohexyl)-6,7- dihydropyrrolo[3,4-f]isoindole-1,3(2H,5H)-dione |
| B126 | 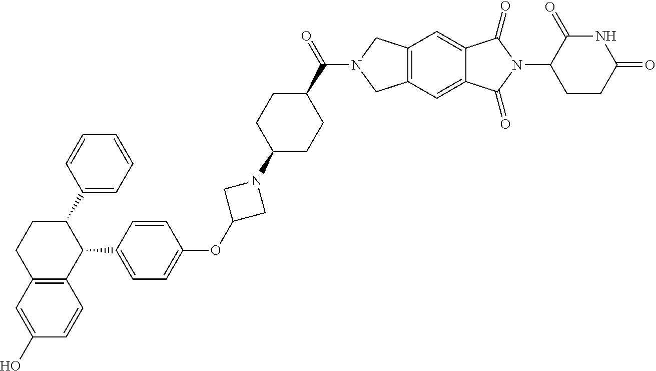 [SMILES: O=C1CCC(N2C(=O)c3cc4c(cc3C2=O)CN(C(=O)[C@H]2CC[C@@H](N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1R,4s)-4-(3-(4-((1R,2S)-6- hydroxy-2-phenyl-1,2,3,4-tetrahydronaphthalen-1- yl)phenoxy)azetidin-1-yl)cyclohexane-1-carbonyl)-6,7- dihydropyrrolo[3,4-f]isoindole-1,3(2H,5H)-dione |
| B127 | 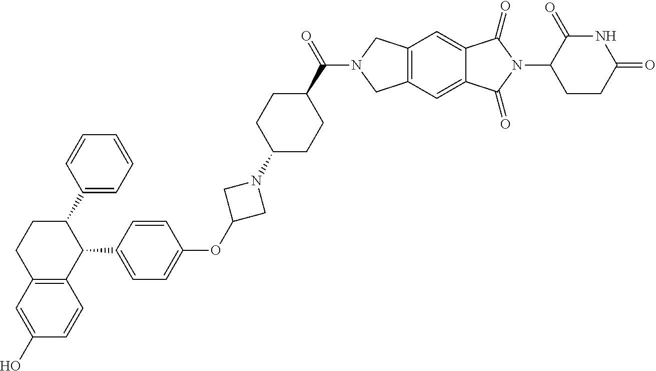 [SMILES: O=C1CCC(N2C(=O)c3cc4c(cc3C2=O)CN(C(=O)[C@H]2CC[C@H](N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1S,4r)-4-(3-(4-((1R,2S)-6- hydroxy-2-phenyl-1,2,3,4-tetrahydronaphthalen-1- yl)phenoxy)azetidin-1-yl)cyclohexane-1-carbonyl)-6,7- dihydropyrrolo[3,4-f]isoindole-1,3(2H,5H)-dione |
| B128 | 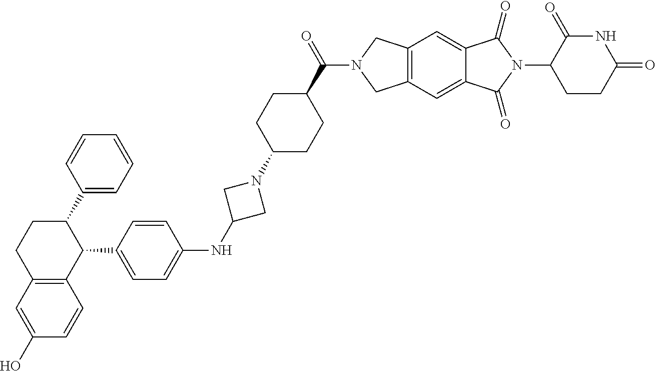 [SMILES: O=C1CCC(N2C(=O)c3cc4c(cc3C2=O)CN(C(=O)[C@H]2CC[C@H](N3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1S,4r)-4-(3-((4-((1R,2S)-6- hydroxy-2-phenyl-1,2,3,4-tetrahydronaphthalen-1- yl)phenyl)amino)azetidin-1-yl)cyclohexane-1-carbonyl)-6,7- dihydropyrrolo[3,4-f]isoindole-1,3(2H,5H)-dione |
| B129 | 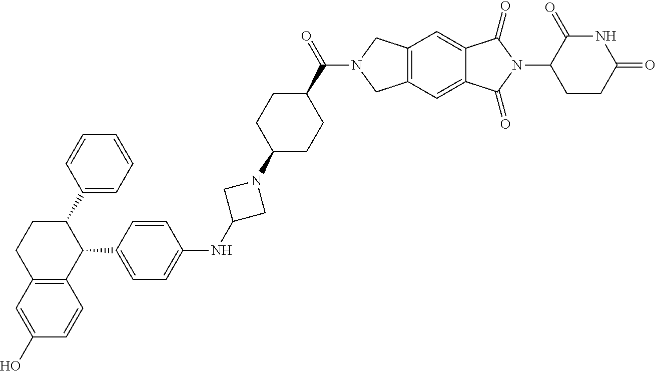 [SMILES: O=C1CCC(N2C(=O)c3cc4c(cc3C2=O)CN(C(=O)[C@H]2CC[C@@H](N3CC(Nc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1R,4s)-4-(3-((4-((1R,2S)-6- hydroxy-2-phenyl-1,2,3,4-tetrahydronaphthalen-1- yl)phenyl)amino)azetidin-1-yl)cyclohexane-1-carbonyl)-6,7- dihydropyrrolo[3,4-f]isoindole-1,3(2H,5H)-dione |
| B130 | 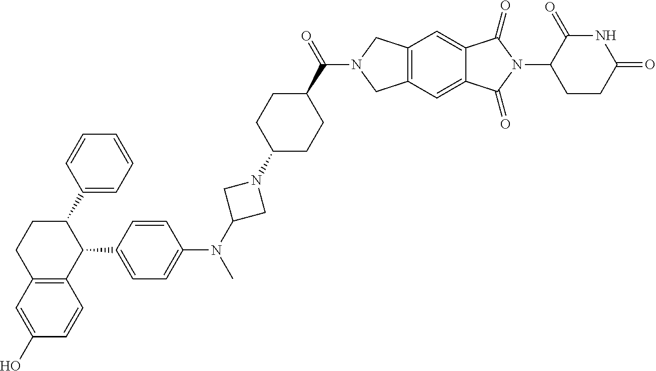 [SMILES: CN(c1ccc([C@@H]2c3ccc(O)cc3CC[C@@H]2c2ccccc2)cc1)C1CN([C@H]2CC[C@H](C(=O)N3Cc4cc5c(cc4C3)C(=O)N(C3CCC(=O)NC3=O)C5=O)CC2)C1] | 2-(2,6-dioxopiperidin-3-yl)-6-((1R,4r)-4-(3-((4-((1R,2S)-6- hydroxy-2-phenyl-1,2,3,4-tetrahydronaphthalen-1- yl)phenyl)(methyl)amino)azetidin-1-yl)cyclohexane-1- carbonyl)-6,7-dihydropyrrolo[3,4-f]isoindole-1,3(2H,5H)- dione |
| B131 | 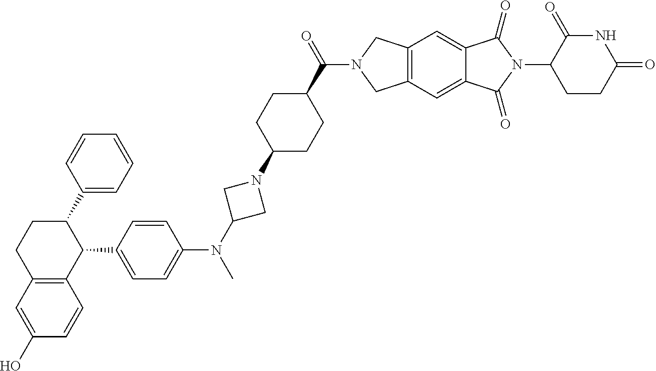 [SMILES: CN(c1ccc([C@@H]2c3ccc(O)cc3CC[C@@H]2c2ccccc2)cc1)C1CN([C@H]2CC[C@@H](C(=O)N3Cc4cc5c(cc4C3)C(=O)N(C3CCC(=O)NC3=O)C5=O)CC2)C1] | 2-(2,6-dioxopiperidin-3-yl)-6-((1S,4s)-4-(3-((4-((1R,2S)-6- hydroxy-2-phenyl-1,2,3,4-tetrahydronaphthalen-1- yl)phenyl)(methyl)amino)azetidin-1-yl)cyclohexane-1- carbonyl)-6,7-dihydropyrrolo[3,4-f]isoindole-1,3(2H,5H)- dione |
| B132 | 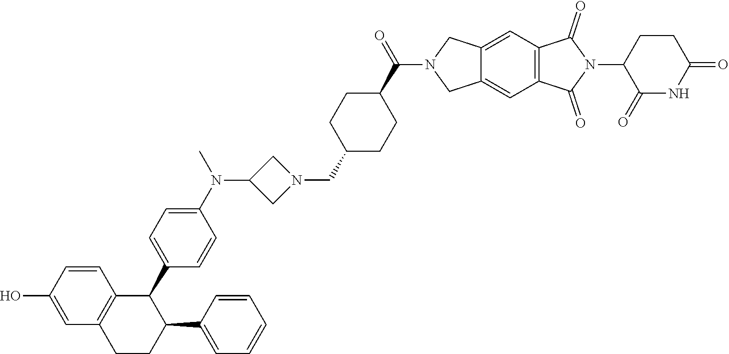 [SMILES: CN(c1ccc([C@@H]2c3ccc(O)cc3CC[C@@H]2c2ccccc2)cc1)C1CN(C[C@H]2CC[C@H](C(=O)N3Cc4cc5c(cc4C3)C(=O)N(C3CCC(=O)NC3=O)C5=O)CC2)C1] | 2-(2,6-dioxopiperidin-3-yl)-6-((1R,4r)-4-((3-((4-((1R,2S)-6- hydroxy-2-phenyl-1,2,3,4-tetrahydronaphthalen-1- yl)phcnyl)(mcthyl)amino)azctidin-1-yl)mcthyl)cyclohexanc-1- carbonyl)-6,7-dihydropyrrolo[3,4-f]isoindole-1,3(2H,5H)- dione |
| B133 | 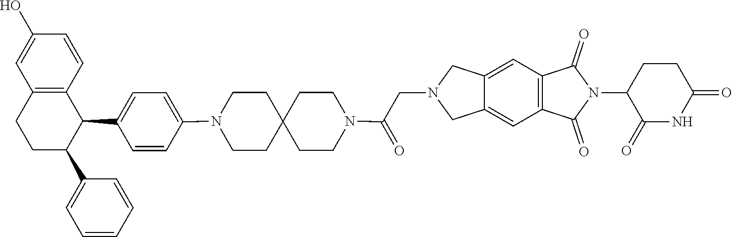 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)C4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)-2-oxoethyl)-6,7- dihydropyrrolo[3,4-f]isoindole-1,3(2H,5H)-dione |
| B136 | 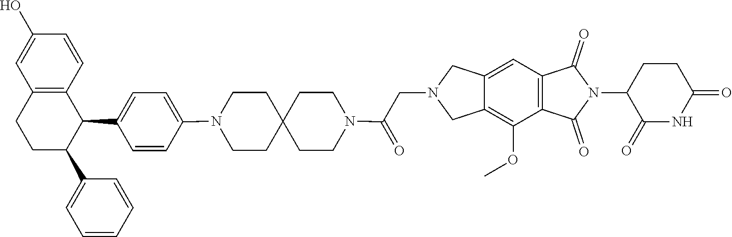 [SMILES: COc1c2c(cc3c1C(=O)N(C1CCC(=O)NC1=O)C3=O)CN(CC(=O)N1CCC3(CC1)CCN(c1ccc([C@@H]4c5ccc(O)cc5CC[C@@H]4c4ccccc4)cc1)CC3)C2] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)-2-oxoethyl)-4-methoxy-6,7- dihydropyrrolo[3,4-f]isoindole-1,3(2H,5H)-dione |
| B138 | 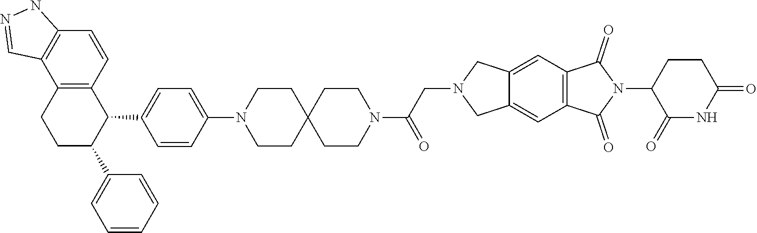 [SMILES: O=C1CCC(N2C(=O)c3cc4c(cc3C2=O)CN(CC(=O)N2CCC3(CC2)CCN(c2ccc([C@H]5c6ccc7[nH]ncc7c6CC[C@H]5c5ccccc5)cc2)CC3)C4)C(=O)N1] | 2-(2,6-dioxopiperidin-3-yl)-6-(2-oxo-2-(9-(4-((6S,7R)-7- phenyl-6,7,8,9-tetrahydro-3H-benzo[e]indazol-6-yl)phenyl)- 3,9-diazaspiro[5.5]undecan-3-yl)ethyl)-6,7-dihydropyrrolo[3,4- f]isoindole-1,3(2H,5H)-dione |
| B139 | 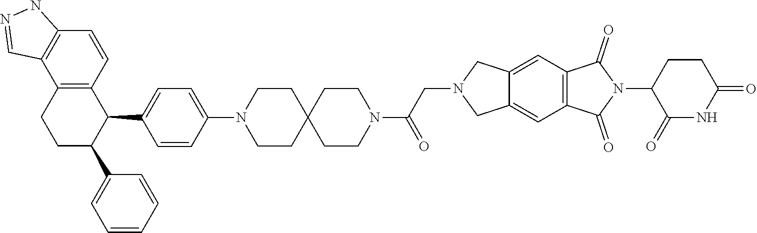 [SMILES: O=C1CCC(N2C(=O)c3cc4c(cc3C2=O)CN(CC(=O)N2CCC3(CC2)CCN(c2ccc([C@@H]5c6ccc7[nH]ncc7c6CC[C@@H]5c5ccccc5)cc2)CC3)C4)C(=O)N1] | 2-(2,6-dioxopiperidin-3-yl)-6-(2-oxo-2-(9-(4-((6R,7S)-7- phenyl-6,7,8,9-tetrahydro-3H-benzo[e]indazol-6-yl)phenyl)- 3,9-diazaspiro[5.5]undecan-3-yl)ethyl)-6,7-dihydropyrrolo[3,4- f]isoindole-1,3(2H,5H)-dione |
| B163 | 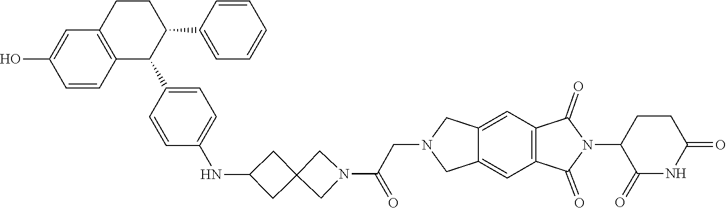 [SMILES: O=C1CCC(N2C(=O)c3cc4c(cc3C2=O)CN(CC(=O)N2CC3(CC(Nc5ccc([C@@H]6c7ccc(O)cc7CC[C@@H]6c6ccccc6)cc5)C3)C2)C4)C(=O)N1] | 2-(2,6-dioxopiperidin-3-yl)-6-(2-(6-((4-((1R,2S)-6-hydroxy-2- phenyl-1,2,3,4-tetrahydronaphthalen-1-yl)phenyl)amino)-2- azaspiro[3.3]heptan-2-yl)-2-oxoethyl)-6,7-dihydropyrrolo[3,4- f]isoindole-1,3(2H,5H)-dione |
| B166 | 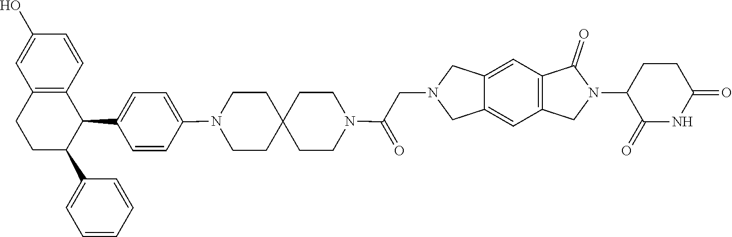 [SMILES: O=C1CCC(N2Cc3cc4c(cc3C2=O)CN(CC(=O)N2CCC3(CC2)CCN(c2ccc([C@@H]5c6ccc(O)cc6CC[C@@H]5c5ccccc5)cc2)CC3)C4)C(=O)N1] | 3-(6-(2-(9-(4-((1R,2S)-6-hydroxy-2-phenyl-1,2,3,4- tetrahydronaphthalen-1-yl)phenyl)-3,9-diazaspiro[5.5]undecan- 3-yl)-2-oxoethyl)-1-oxo-3,5,6,7-tetrahydropyrrolo[3,4- f]isoindol-2(1H)-yl)piperidine-2,6-dione |
| B179 | 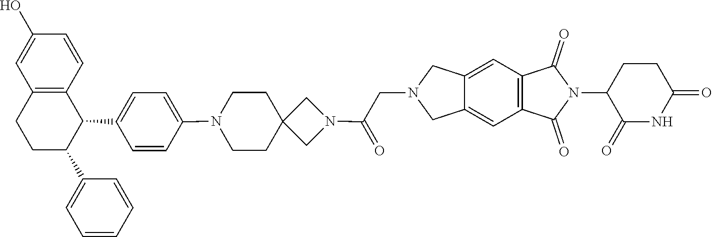 [SMILES: O=C1CCC(N2C(=O)c3cc4c(cc3C2=O)CN(CC(=O)N2CC3(CCN(c5ccc([C@H]6c7ccc(O)cc7CC[C@H]6c6ccccc6)cc5)CC3)C2)C4)C(=O)N1] | 2-(2,6-dioxopiperidin-3-yl)-6-(2-(7-(4-((1S,2R)-6-hydroxy-2- phenyl-1,2,3,4-tetrahydronaphthalen-1-yl)phenyl)-2,7- diazaspiro[3.5]nonan-2-yl)-2-oxoethyl)-6,7- dihydropyrrolo[3,4-f]isoindole-1,3(2H,5H)-dione |
| B180 | 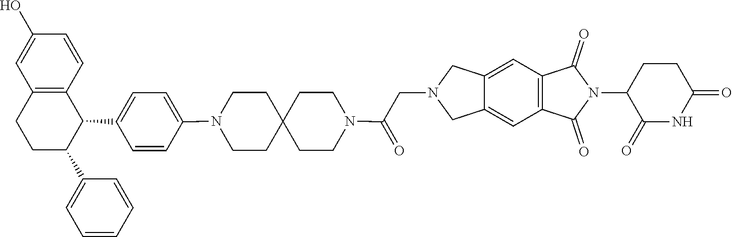 [SMILES: O=C1CCC(N2C(=O)c3cc4c(cc3C2=O)CN(CC(=O)N2CCC3(CC2)CCN(c2ccc([C@H]5c6ccc(O)cc6CC[C@H]5c5ccccc5)cc2)CC3)C4)C(=O)N1] | 2-(2,6-dioxopiperidin-3-yl)-6-(2-(9-(4-((1S,2R)-6-hydroxy-2- phenyl-1,2,3,4-tetrahydronaphthalen-1-yl)phenyl)-3,9- diazaspiro[5.5]undecan-3-yl)-2-oxoethyl)-6,7- dihydropyrrolo[3,4-f]isoindole-1,3(2H,5H)-dione |
| B181 | 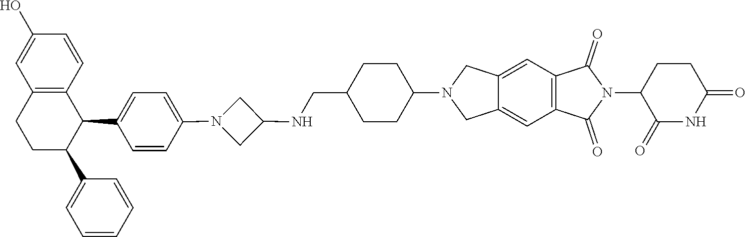 [SMILES: O=C1CCC(N2C(=O)c3cc4c(cc3C2=O)CN(C2CCC(CNC3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-(4-(((1-(4-((1R,2S)-6-hydroxy-2- phenyl-1,2,3,4-tetrahydronaphthalen-1-yl)phenyl)azetidin-3- yl)amino)methyl)cyclohexyl)-6,7-dihydropyrrolo[3,4- f]isoindole-1,3(2H,5H)-dione |
| B182* | 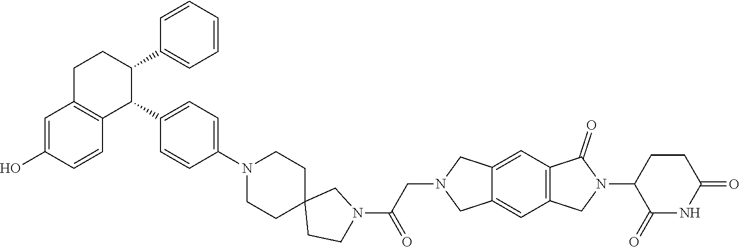 [SMILES: O=C1CCC(N2Cc3cc4c(cc3C2=O)CN(CC(=O)N2CCC3(CCN(c5ccc([C@@H]6c7ccc(O)cc7CC[C@@H]6c6ccccc6)cc5)CC3)C2)C4)C(=O)N1] | 3-(6-(2-(8-(4-((1R,2S)-6-hydroxy-2-phenyl-1,2,3,4- tetrahydronaphthalen-1-yl)phenyl)-2,8-diazaspiro[4.5]decan-2- yl)-2-oxoethyl)-1-oxo-3,5,6,7-tetrahydropyrrolo[3,4-f]isoindol- 2(1H)-yl)piperidine-2,6-dione |
| B183* | 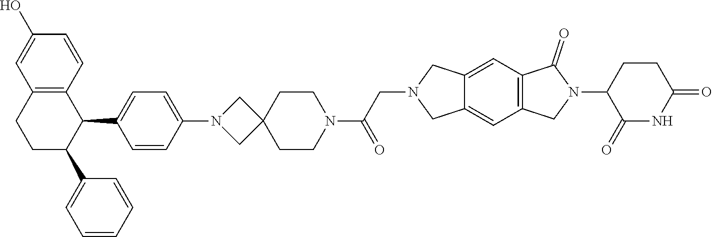 [SMILES: O=C1CCC(N2Cc3cc4c(cc3C2=O)CN(CC(=O)N2CCC3(CC2)CN(c2ccc([C@@H]5c6ccc(O)cc6CC[C@@H]5c5ccccc5)cc2)C3)C4)C(=O)N1] | 3-(6-(2-(2-(4-((1R,2S)-6-hydroxy-2-phenyl-1,2,3,4- tetrahydronaphthalen-1-yl)phenyl)-2,7-diazaspiro[3.5]nonan-7- yl)-2-oxoethyl)-1-oxo-3,5,6,7-tetrahydropyrrolo[3,4-f]isoindol- 2(1H)-yl)piperidine-2,6-dione |
| B184* | 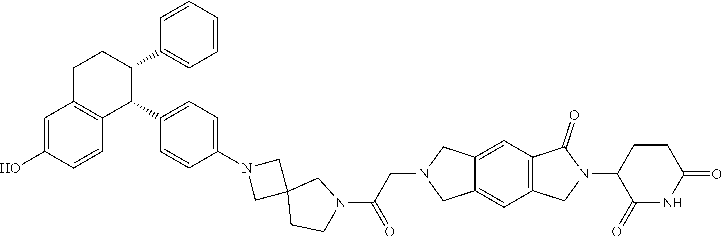 [SMILES: O=C1CCC(N2Cc3cc4c(cc3C2=O)CN(CC(=O)N2CCC3(C2)CN(c2ccc([C@@H]5c6ccc(O)cc6CC[C@@H]5c5ccccc5)cc2)C3)C4)C(=O)N1] | 3-(6-(2-(2-(4-((1R,2S)-6-hydroxy-2-phenyl-1,2,3,4- tetrahydronaphthalen-1-yl)phenyl)-2,6-diazaspiro[3.4]octan-6- yl)-2-oxoethyl)-1-oxo-3,5,6,7-tetrahydropyrrolo[3,4-f]isoindol- 2(1H)-yl)piperidine-2,6-dione |
| B185* | 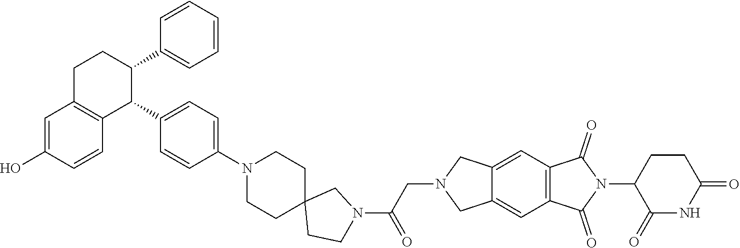 [SMILES: O=C1CCC(N2C(=O)c3cc4c(cc3C2=O)CN(CC(=O)N2CCC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(8-(4-((1R,2S)-6-hydroxy-2- phenyl-1,2,3,4-tetrahydronaphthalen-1-yl)phenyl)-2,8- diazaspiro[4.5]decan-2-yl)-2-oxoethyl)-6,7- dihydropyrrolo[3,4-f]isoindole-1,3(2H,5H)-dione |
| B186* | 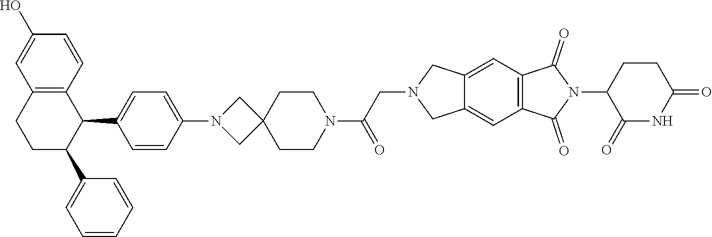 [SMILES: O=C1CCC(N2C(=O)c3cc4c(cc3C2=O)CN(CC(=O)N2CCC3(CC2)CN(c2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4-((1R,2S)-6-hydroxy-2- phenyl-1,2,3,4-tetrahydronaphthalen-1-yl)phenyl)-2,7- diazaspiro[3.5]nonan-7-yl)-2-oxoethyl)-6,7- dihydropyrrolo[3,4-f]isoindole-1,3(2H,5H)-dione |
| B187* | 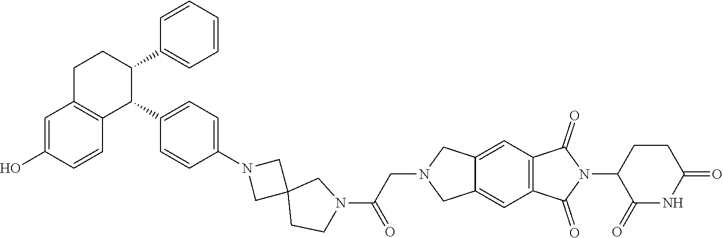 [SMILES: O=C1CCC(N2C(=O)c3cc4c(cc3C2=O)CN(CC(=O)N2CCC3(C2)CN(c2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4-((1R,2S)-6-hydroxy-2- phenyl-1,2,3,4-tetrahydronaphthalen-1-yl)phenyl)-2,6- diazaspiro[3.4]octan-6-yl)-2-oxoethyl)-6,7-dihydropyrrolo[3,4- f]isoindole-1,3(2H,5H)-dione |
| B188* | 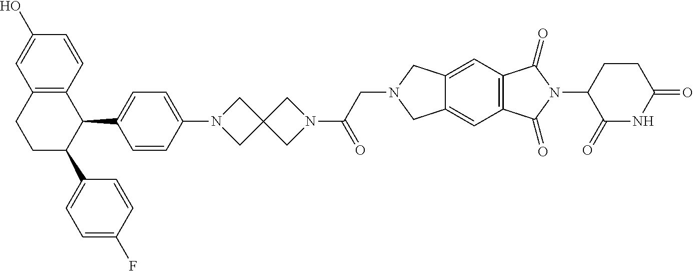 [SMILES: O=C1CCC(N2C(=O)c3cc4c(cc3C2=O)CN(CC(=O)N2CC3(C2)CN(c2ccc([C@@H]5c6ccc(O)cc6CC[C@@H]5c5ccc(F)cc5)cc2)C3)C4)C(=O)N1] | 2-(2,6-dioxopipcridin-3-yl)-6-(2-(6-(4-((1R,2S)-2-(4- fluorophenyl)-6-hydroxy-1,2,3,4-tetrahydronaphthalen-1- yl)phenyl)-2,6-diazaspiro[3.3]heptan-2-yl)-2-oxoethyl)-6,7- dihydropyrrolo[3,4-f]isoindole-1,3(2H,5H)-dione |
| B189 | 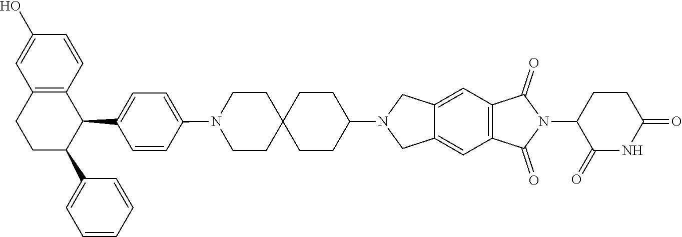 [SMILES: O=C1CCC(N2C(=O)c3cc4c(cc3C2=O)CN(C2CCC3(CC2)CCN(c2ccc([C@@H]5c6ccc(O)cc6CC[C@@H]5c5ccccc5)cc2)CC3)C4)C(=O)N1] | 2-(2,6-dioxopiperidin-3-yl)-6-(3-(4-((1R,2S)-6-hydroxy-2- phenyl-1,2,3,4-tetrahydronaphthalen-1-yl)phenyl)-3- azaspiro[5.5]undecan-9-yl)-6,7-dihydropyrrolo[3,4-f]isoindole- 1,3(2H,5H)-dione |
| B190* | 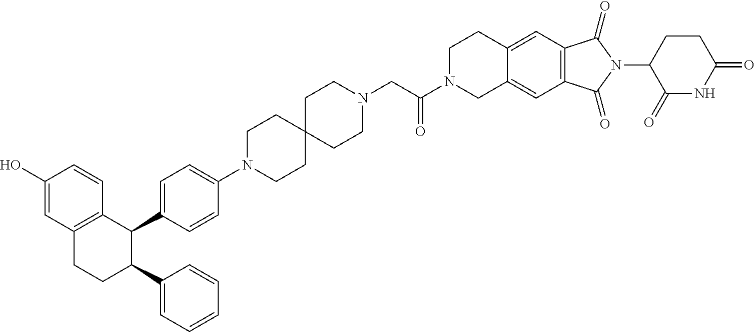 [SMILES: O=C1CCC(N2C(=O)c3cc4c(cc3C2=O)CN(C(=O)CN2CCC3(CC2)CCN(c2ccc([C@@H]5c6ccc(O)cc6CC[C@@H]5c5ccccc5)cc2)CC3)CC4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)acetyl)-5,6,7,8-tetrahydro-1H- pyrrolo[3,4-g]isoquinoline-1,3(2H)-dione |
| B191* | 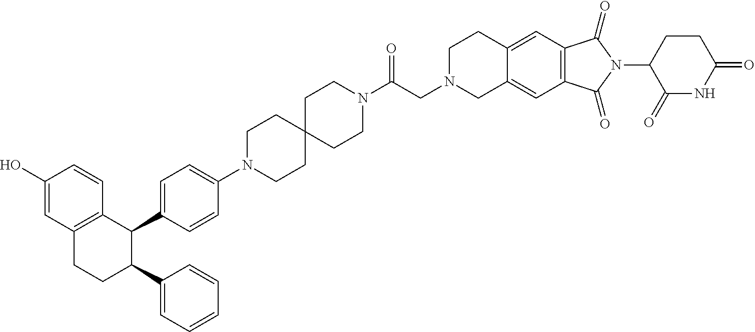 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)CC4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)-2-oxoethyl)-5,6,7,8-tetrahydro- 1H-pyrrolo[3,4-g]isoquinoline-1,3(2H)-dione |
| B192* | 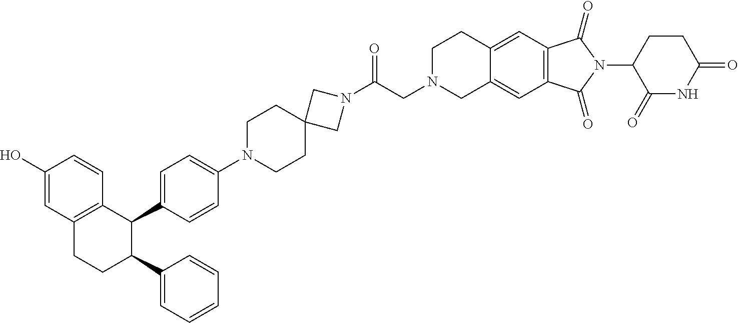 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5)CC3)C2)CC4)C(=O)N1] | 2-(2,6-dioxopiperidin-3-yl)-6-(2-(7-(4-((1R,2S)-6-hydroxy-2- phenyl-1,2,3,4-tetrahydronaphthalen-1-yl)phenyl)-2,7- diazaspiro[3.5]nonan-2-yl)-2-oxoethyl)-5,6,7,8-tetrahydro-1H- pyrrolo[3,4-g]isoquinoline-1,3(2H)-dione |
| B193* | 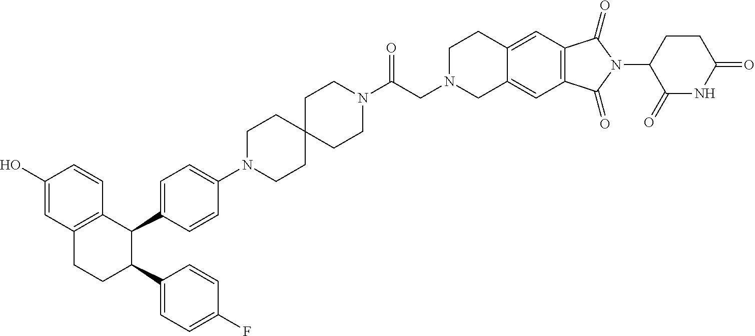 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccc(F)cc5)cc2)CC3)CC4)C(=O)N1] | 2-(2,6-dioxopiperidin-3-yl)-6-(2-(9-(4-((1R,2S)-2-(4- fluorophenyl)-6-hydroxy-1,2,3,4-tetrahydronaphthalen-1- yl)phenyl)-3,9-diazaspiro[5.5]undecan-3-yl)-2-oxoethyl)- 5,6,7,8-tetrahydro-1H-pyrrolo[3,4-g]isoquinoline-1,3(2H)- dione |
| B194* | 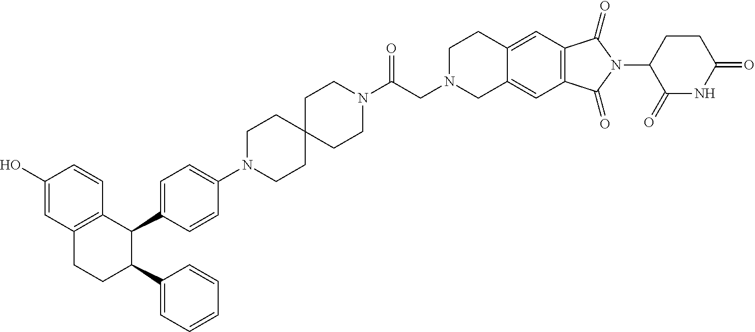 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)CC4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)-2-oxoethyl)-5,6,7,8-tetrahydro- 1H-pyrrolo[3,4-g]isoquinoline-1,3(2H)-dione |
| B195* | 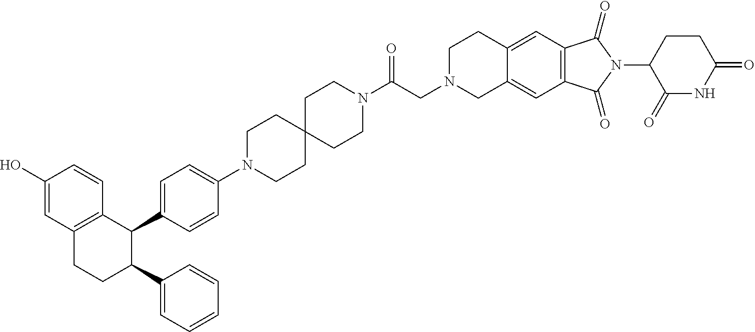 [SMILES: O=C1CCC(N2C(=O)c3cc4c(cc3C2=O)CN(CC(=O)N2CCC3(CC2)CCN(c2ccc([C@@H]5c6ccc(O)cc6CC[C@@H]5c5ccccc5)cc2)CC3)CC4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)-2-oxoethyl)-5,6,7,8-tetrahydro- 1H-pyrrolo[3,4-g]isoquinoline-1,3(2H)-dione |
| B196* | 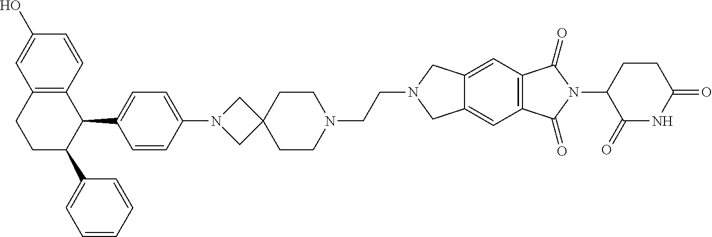 [SMILES: O=C1CCC(N2C(=O)c3cc4c(cc3C2=O)CN(CCN2CCC3(CC2)CN(c2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4-((1R,2S)-6-hydroxy-2- phenyl-1,2,3,4-tetrahydronaphthalen-1-yl)phenyl)-2,7- diazaspiro[3.5]nonan-7-yl)ethyl)-6,7-dihydropyrrolo[3,4- f]isoindole-1,3(2H,5H)-dione |
| B197* | 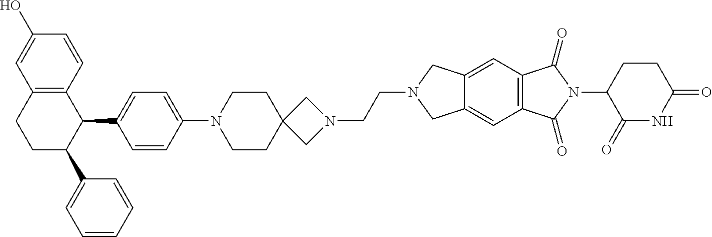 [SMILES: O=C1CCC(N2C(=O)c3cc4c(cc3C2=O)CN(CCN2CC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-(2-(7-(4-((1R,2S)-6-hydroxy-2- phenyl-1,2,3,4-tetrahydronaphthalen-1-yl)phenyl)-2,7- diazaspiro[3.5]nonan-2-yl)ethyl)-6,7-dihydropyrrolo[3,4- f]isoindole-1,3(2H,5H)-dione |
| B198* | 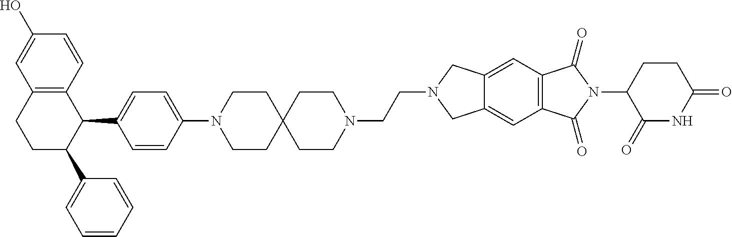 [SMILES: O=C1CCC(N2C(=O)c3cc4c(cc3C2=O)CN(CCN2CCC3(CC2)CCN(c2ccc([C@@H]5c6ccc(O)cc6CC[C@@H]5c5ccccc5)cc2)CC3)C4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-3,9- diazaspiro[5.5]undecan-3-yl)ethyl)-6,7-dihydropyrrolo[3,4- f]isoindole-1,3(2H,5H)-dione |
| B199 | 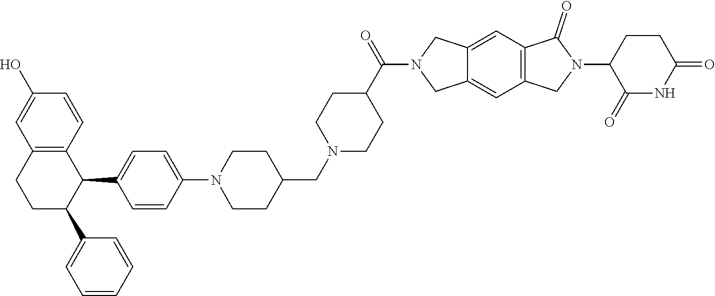 [SMILES: O=C1CCC(N2Cc3cc4c(cc3C2=O)CN(C(=O)C2CCN(CC3CCN(c5ccc([C@@H]6c7ccc(O)cc7CC[C@@H]6c6ccccc6)cc5)CC3)CC2)C4)C(=O)N1] | 3-(6-(1-((1-(4-((1R,2S)-6-hydroxy-2-phenyl-1,2,3,4- tetrahydronaphthalen-1-yl)phenyl)piperidin-4- yl)methyl)piperidine-4-carbonyl)-1-oxo-3,5,6,7- tetrahydropyrrolo[3,4-f]isoindol-2(1H)-yl)piperidine-2,6-dione |
| B200 | 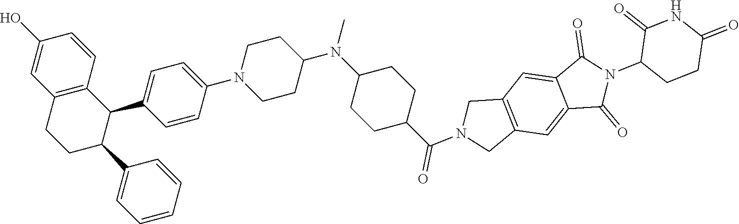 [SMILES: CN(C1CCC(C(=O)N2Cc3cc4c(cc3C2)C(=O)N(C2CCC(=O)NC2=O)C4=O)CC1)C1CCN(c2ccc([C@@H]3c4ccc(O)cc4CC[C@@H]3c3ccccc3)cc2)CC1] | 2-(2,6-dioxopiperidin-3-yl)-6-(4-((1-(4-((1R,2S)-6-hydroxy-2- phenyl-1,2,3,4-tetrahydronaphthalen-1-yl)phenyl)piperidin-4- yl)(methyl)amino)cyclohexane-1-carbonyl)-6,7- dihydropyrrolo[3,4-f]isoindole-1,3(2H,5H)-dione |
| B201 | 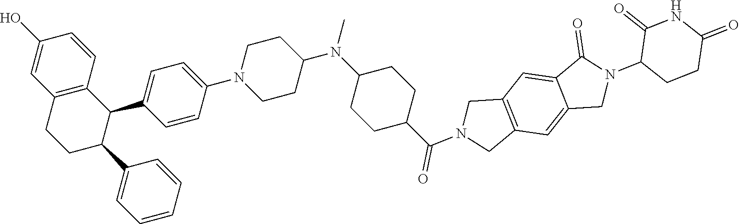 [SMILES: CN(C1CCC(C(=O)N2Cc3cc4c(cc3C2)C(=O)N(C2CCC(=O)NC2=O)C4)CC1)C1CCN(c2ccc([C@@H]3c4ccc(O)cc4CC[C@@H]3c3ccccc3)cc2)CC1] | 3-(6-(4-((1-(4-((1R,2S)-6-hydroxy-2-phenyl-1,2,3,4- tetrahydronaphthalen-1-yl)phenyl)piperidin-4- yl)(methyl)amino)cyclohexane-1-carbonyl)-1-oxo-3,5,6,7- tetrahydropyrrolo[3,4-f]isoindol-2(1H)-yl)piperidine-2,6-dione |
| B202 | 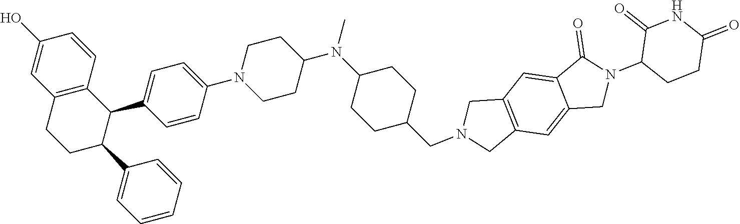 [SMILES: CN(C1CCC(CN2Cc3cc4c(cc3C2)C(=O)N(C2CCC(=O)NC2=O)C4)CC1)C1CCN(c2ccc([C@@H]3c4ccc(O)cc4CC[C@@H]3c3ccccc3)cc2)CC1] | 3-(6-((4-((1-(4-((1R,2S)-6-hydroxy-2-phenyl-1,2,3,4- tetrahydronaphthalen-1-yl)phenyl)piperidin-4- yl)(methyl)amino)cyclohexyl)methyl)-1-oxo-3,5,6,7- tetrahydropyrrolo[3,4-f]isoindol-2(1H)-yl)piperidine-2,6-dione |
| B203 | 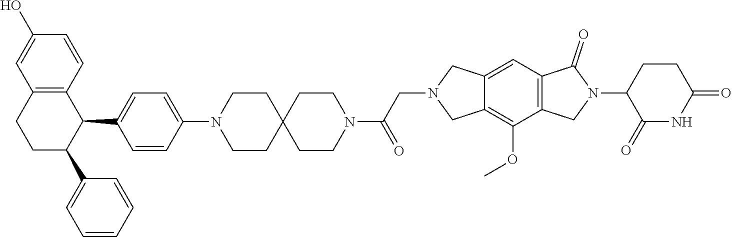 [SMILES: COc1c2c(cc3c1CN(C1CCC(=O)NC1=O)C3=O)CN(CC(=O)N1CCC3(CC1)CCN(c1ccc([C@@H]4c5ccc(O)cc5CC[C@@H]4c4ccccc4)cc1)CC3)C2] | 3-(6-(2-(9-(4-((1R,2S)-6-hydroxy-2-phenyl-1,2,3,4- tetrahydronaphthalen-1-yl)phenyl)-3,9-diazaspiro[5.5]undecan- 3-yl)-2-oxoethyl)-4-methoxy-1-oxo-3,5,6,7- tetrahydropyrrolo[3,4-f]isoindol-2(1H)-yl)piperidine-2,6-dione |
| B210 | 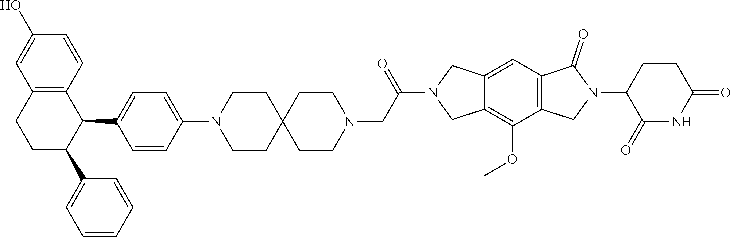 [SMILES: COc1c2c(cc3c1CN(C1CCC(=O)NC1=O)C3=O)CN(C(=O)CN1CCC3(CC1)CCN(c1ccc([C@@H]4c5ccc(O)cc5CC[C@@H]4c4ccccc4)cc1)CC3)C2] | 3-(6-(2-(9-(4-((1R,2S)-6-hydroxy-2-phenyl-1,2,3,4- tetrahydronaphthalen-1-yl)phenyl)-3,9-diazaspiro[5.5]undecan- 3-yl)acetyl)-4-methoxy-1-oxo-3,5,6,7-tetrahydropyrrolo[3,4- f]isoindol-2(1H)-yl)piperidine-2,6-dione |
| B217 | 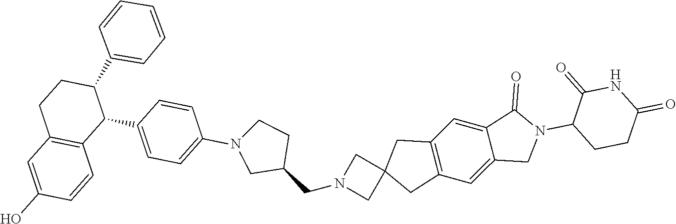 [SMILES: O=C1CCC(N2Cc3cc4c(cc3C2=O)CC2(C4)CN(C[C@@H]3CCN(c4ccc([C@@H]5c6ccc(O)cc6CC[C@@H]5c5ccccc5)cc4)C3)C2)C(=O)N1] | 3-(1-(((S)-1-(4-((1R,2S)-6-hydroxy-2-phenyl-1,2,3,4- tetrahydronaphthalen-1-yl)phenyl)pyrrolidin-3-yl)methyl)-1′- oxo-5′,7′-dihydro-1'H-spiro[azetidine-3,6′- cyclopenta[f]isoindol]-2'(3'H)-yl)piperidine-2,6-dione |
| B235 | 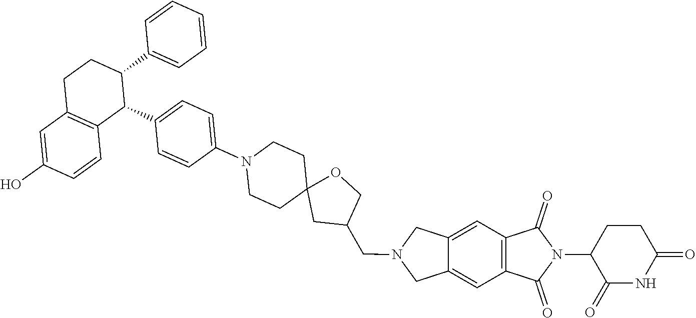 [SMILES: O=C1CCC(N2C(=O)c3cc4c(cc3C2=O)CN(CC2COC3(CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((8-(4-((1R,2S)-6-hydroxy-2- phenyl-1,2,3,4-tetrahydronaphthalen-1-yl)phenyl)-1-oxa-8- azaspiro[4.5]decan-3-yl)methyl)-6,7-dihydropyrrolo[3,4- f]isoindole-1,3(2H,5H)-dione |
| B259* | 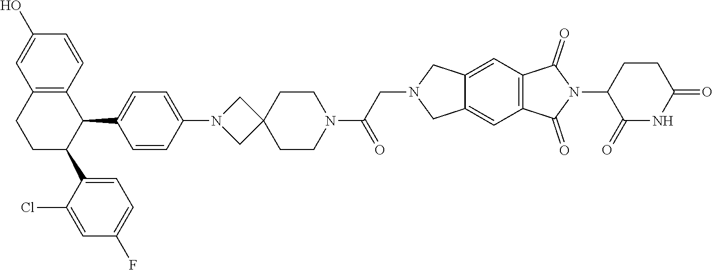 [SMILES: O=C1CCC(N2C(=O)c3cc4c(cc3C2=O)CN(CC(=O)N2CCC3(CC2)CN(c2ccc([C@@H]5c6ccc(O)cc6CC[C@@H]5c5ccc(F)cc5Cl)cc2)C3)C4)C(=O)N1] | 6-(2-(2-(4-((1R,2S)-2-(2-chloro-4-fluorophenyl)-6-hydroxy- 1,2,3,4-tetrahydronaphthalen-1-yl)phenyl)-2,7- diazaspiro[3.5]nonan-7-yl)-2-oxoethyl)-2-(2,6-dioxopiperidin- 3-yl)-6,7-dihydropyrrolo[3,4-f]isoindole-1,3(2H,5H)-dione |
| B260* | 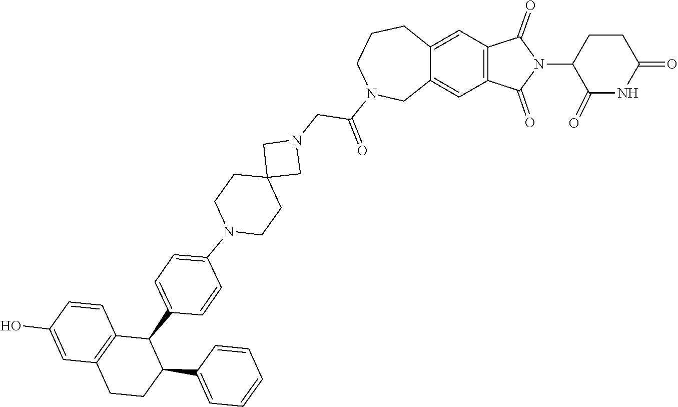 [SMILES: O=C1CCC(N2C(=O)c3cc4c(cc3C2=O)CN(C(=O)CN2CC3(CCN(c5ccc([C@@H]6c7ccc(O)cc7CC[C@@H]6c6ccccc6)cc5)CC3)C2)CCC4)C(=O)N1] | 2-(2,6-dioxopiperidin-3-yl)-6-(2-(7-(4-((1R,2S)-6-hydroxy-2- phenyl-1,2,3,4-tetrahydronaphthalen-1-yl)phenyl)-2,7- diazaspiro[3.5]nonan-2-yl)acetyl)-6,7,8,9- tetrahydroazepino[3,4-f]isoindole-1,3(2H,5H)-dione |
| B261* | 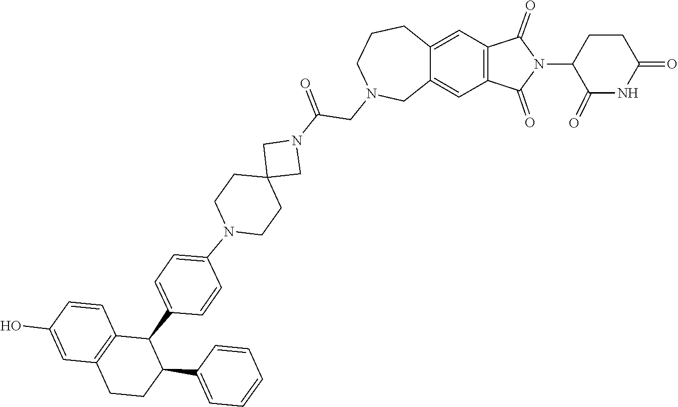 [SMILES: O=C1CCC(N2C(=O)c3cc4c(cc3C2=O)CN(CC(=O)N2CC3(CCN(c5ccc([C@@H]6c7ccc(O)cc7CC[C@@H]6c6ccccc6)cc5)CC3)C2)CCC4)C(=O)N1] | 2-(2,6-dioxopiperidin-3-yl)-6-(2-(7-(4-((1R,2S)-6-hydroxy-2- phenyl-1,2,3,4-tetrahydronaphthalen-1-yl)phenyl)-2,7- diazaspiro[3.5]nonan-2-yl)-2-oxoethyl)-6,7,8,9- tetrahydroazepino[3,4-f]isoindole-1,3(2H,5H)-dione |
| B262 | 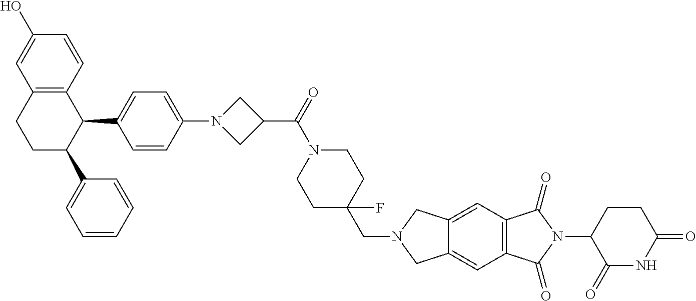 [SMILES: O=C1CCC(N2C(=O)c3cc4c(cc3C2=O)CN(CC2(F)CCN(C(=O)C3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((4-fluoro-1-(1-(4-((1R,2S)-6- hydroxy-2-phenyl-1,2,3,4-tetrahydronaphthalen-1- yl)phenyl)azetidine-3-carbonyl)piperidin-4-yl)methyl)-6,7- dihydropyrrolo[3,4-f]isoindole-1,3(2H,5H)-dione |
| B263 | 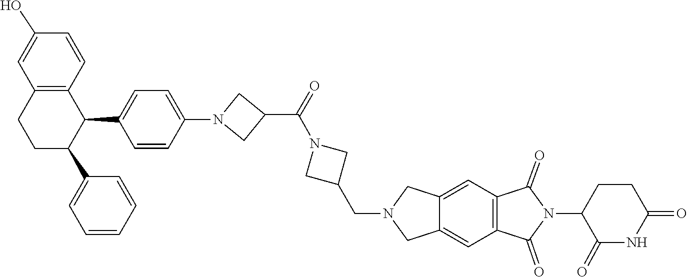 [SMILES: O=C1CCC(N2C(=O)c3cc4c(cc3C2=O)CN(CC2CN(C(=O)C3CN(c5ccc([C@@H]6c7ccc(O)cc7CC[C@@H]6c6ccccc6)cc5)C3)C2)C4)C(=O)N1] | 2-(2,6-dioxopiperidin-3-yl)-6-((1-(1-(4-((1R,2S)-6-hydroxy-2- phenyl-1,2,3,4-tetrahydronaphthalen-1-yl)phenyl)azetidine-3- carbonyl)azetidin-3-yl)methyl)-6,7-dihydropyrrolo[3,4- f]isoindole-1,3(2H,5H)-dione |
| B264 | 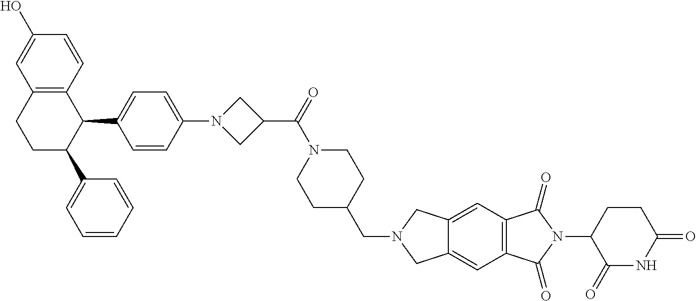 [SMILES: O=C1CCC(N2C(=O)c3cc4c(cc3C2=O)CN(CC2CCN(C(=O)C3CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-(1-(4-((1R,2S)-6-hydroxy-2- phenyl-1,2,3,4-tetrahydronaphthalen-1-yl)phenyl)azetidine-3- carbonyl)piperidin-4-yl)methyl)-6,7-dihydropyrrolo[3,4- f]isoindole-1,3(2H,5H)-dione |
| B265 | 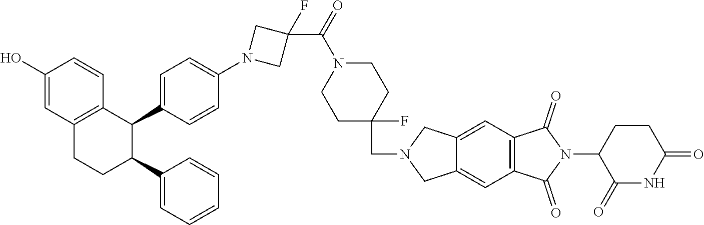 [SMILES: O=C1CCC(N2C(=O)c3cc4c(cc3C2=O)CN(CC2(F)CCN(C(=O)C3(F)CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((4-fluoro-1-(3-fluoro-1-(4- ((1R,2S)-6-hydroxy-2-phenyl-1,2,3,4-tetrahydronaphthalen-1- yl)phenyl)azetidine-3-carbonyl)piperidin-4-yl)methyl)-6,7- dihydropyrrolo[3,4-f]isoindole-1,3(2H,5H)-dione |
| B266 | 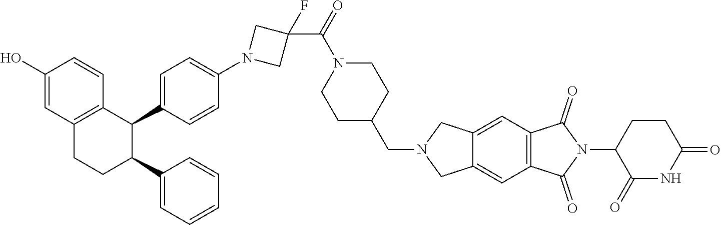 [SMILES: O=C1CCC(N2C(=O)c3cc4c(cc3C2=O)CN(CC2CCN(C(=O)C3(F)CN(c5ccc([C@@H]6c7ccc(O)cc7CC[C@@H]6c6ccccc6)cc5)C3)CC2)C4)C(=O)N1] | 2-(2,6-dioxopiperidin-3-yl)-6-((1-(3-fluoro-1-(4-((1R,2S)-6- hydroxy-2-phenyl-1,2,3,4-tetrahydronaphthalen-1- yl)phenyl)azetidine-3-carbonyl)piperidin-4-yl)methyl)-6,7- dihydropyrrolo[3,4-f]isoindolc-1,3(2H,5H)-dionc |
| B267 | 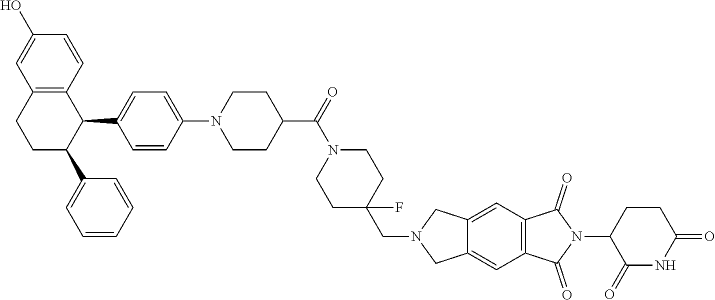 [SMILES: O=C1CCC(N2C(=O)c3cc4c(cc3C2=O)CN(CC2(F)CCN(C(=O)C3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((4-fluoro-1-(1-(4-((1R,2S)-6- hydroxy-2-phenyl-1,2,3,4-tetrahydronaphthalen-1- yl)phenyl)piperidine-4-carbonyl)piperidin-4-yl)methyl)-6,7- dihydropyrrolo[3,4-f]isoindole-1,3(2H,5H)-dione |
| B268 | 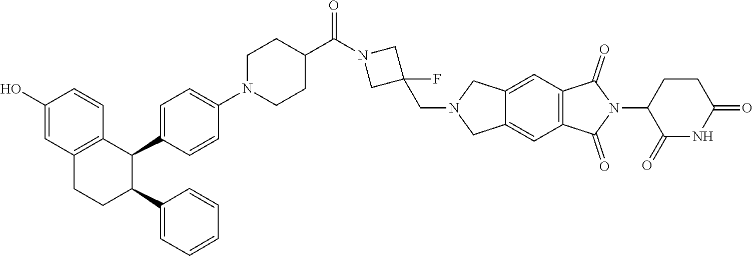 [SMILES: O=C1CCC(N2C(=O)c3cc4c(cc3C2=O)CN(CC2(F)CN(C(=O)C3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((3-fluoro-1-(1-(4-((1R,2S)-6- hydroxy-2-phenyl-1,2,3,4-tetrahydronaphthalen-1- yl)phenyl)piperidine-4-carbonyl)azetidin-3-yl)methyl)-6,7- dihydropyrrolo[3,4-f]isoindole-1,3(2H,5H)-dione |
| B269 | 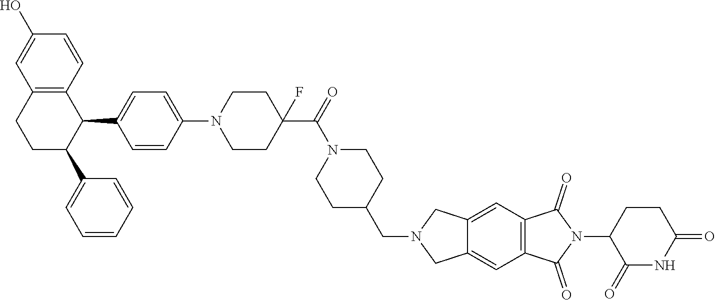 [SMILES: O=C1CCC(N2C(=O)c3cc4c(cc3C2=O)CN(CC2CCN(C(=O)C3(F)CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1-(4-fluoro-1-(4-((1R,2S)-6- hydroxy-2-phenyl-1,2,3,4-tetrahydronaphthalen-1- yl)phenyl)piperidine-4-carbonyl)piperidin-4-yl)methyl)-6,7- dihydropyrrolo[3,4-f]isoindole-1,3(2H,5H)-dione |
| B270 | 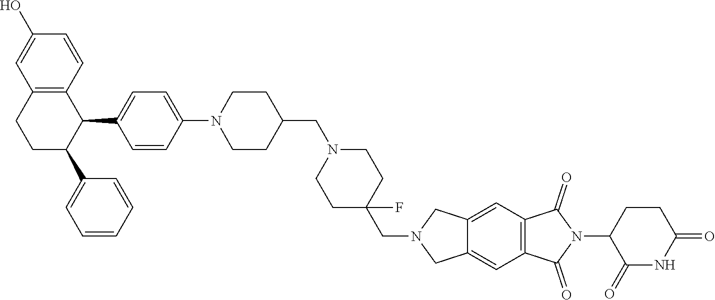 [SMILES: O=C1CCC(N2C(=O)c3cc4c(cc3C2=O)CN(CC2(F)CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((4-fluoro-1-((1-(4-((1R,2S)-6- hydroxy-2-phenyl-1,2,3,4-tetrahydronaphthalen-1- yl)phenyl)piperidin-4-yl)methyl)piperidin-4-yl)methyl)-6,7- dihydropyrrolo[3,4-f]isoindole-1,3(2H,5H)-dione |
| B271 | 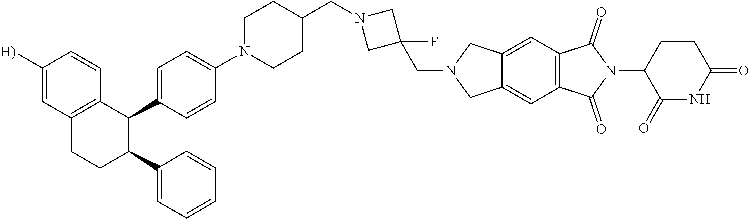 | 2-(2,6-dioxopiperidin-3-yl)-6-((3-fluoro-1-((1-(4-((1R,2S)-6- hydroxy-2-phenyl-1,2,3,4-tetrahydronaphthalen-1- yl)phenyl)piperidin-4-yl)methyl)azetidin-3-yl)methyl)-6,7- dihydropyrrolo[3,4-f]isoindole-1,3(2H,5H)-dione |
| B272 | 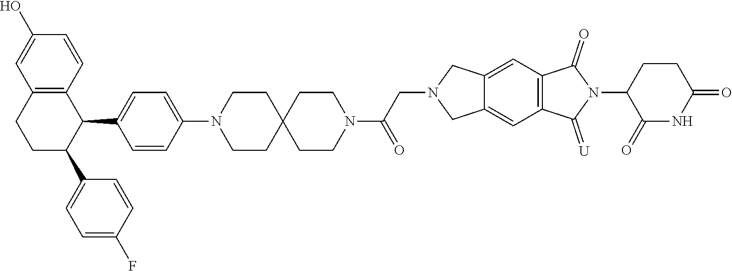 | 2-(2,6-dioxopiperidin-3-yl)-6-(2-(9-(4-((1R,2S)-2-(4- fluorophenyl)-6-hydroxy-1,2,3,4-tetrahydronaphthalen-1- yl)phenyl)-3,9-diazaspiro[5.5]undecan-3-yl)-2-oxoethyl)-6,7- dihydropyrrolo[3,4-f]isoindole-1,3(2H,5H)-dione |
| B273 | 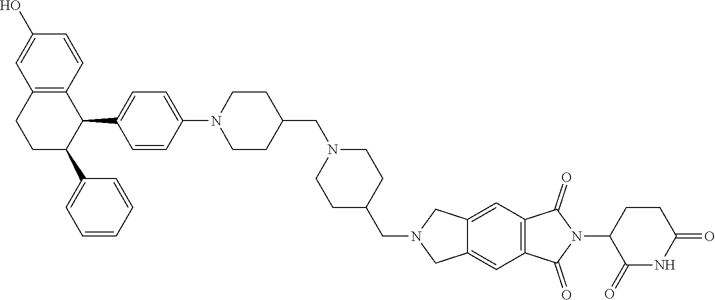 [SMILES: O=C1CCC(N2C(=O)c3cc4c(cc3C2=O)CN(CC2CCN(CC3CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-((1-((1-(4-((1R,2S)-6-hydroxy-2- phenyl-1,2,3,4-tetrahydronaphthalen-1-yl)phenyl)piperidin-4- yl)methyl)piperidin-4-yl)methyl)-6,7-dihydropyrrolo[3,4- f]isoindole-1,3(2H,5H)-dione |
| B274 | 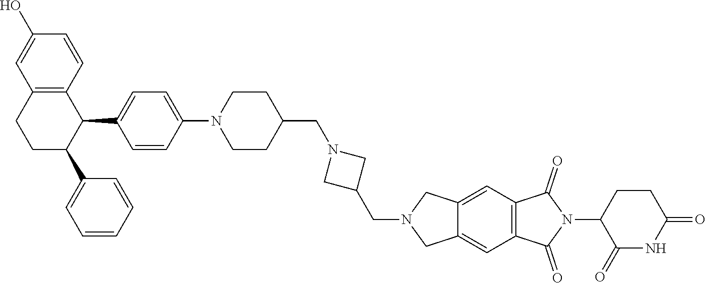 [SMILES: O=C1CCC(N2C(=O)c3cc4c(cc3C2=O)CN(CC2CN(CC3CCN(c5ccc([C@@H]6c7ccc(O)cc7CC[C@@H]6c6ccccc6)cc5)CC3)C2)C4)C(=O)N1] | 2-(2,6-dioxopiperidin-3-yl)-6-((1-((1-(4-((1R,2S)-6-hydroxy-2- phenyl-1,2,3,4-tetrahydronaphthalen-1-yl)phenyl)piperidin-4- yl)methyl)azetidin-3-yl)methyl)-6,7-dihydropyrrolo[3,4- f]isoindole-1,3(2H,5H)-dione |
| B275 | 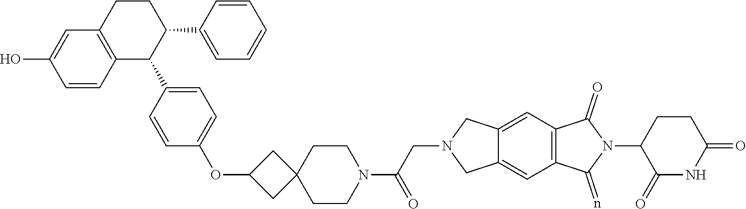 | 2-(2,6-dioxopiperidin-3-yl)-6-(2-(2-(4-((1R,2S)-6-hydroxy-2- phenyl-1,2,3,4-tetrahydronaphthalen-1-yl)phenoxy)-7- azaspiro[3.5]nonan-7-yl)-2-oxoethyl)-6,7-dihydropyrrolo[3,4- f]isoindole-1,3(2H,5H)-dione |
| B276 | 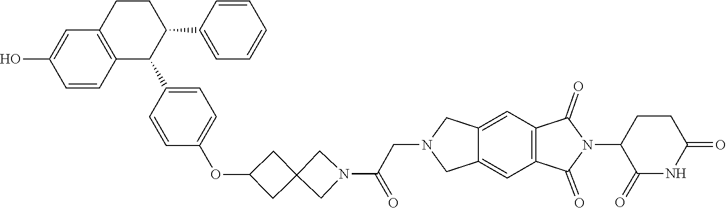 [SMILES: O=C1CCC(N2C(=O)c3cc4c(cc3C2=O)CN(CC(=O)N2CC3(CC(Oc5ccc([C@@H]6c7ccc(O)cc7CC[C@@H]6c6ccccc6)cc5)C3)C2)C4)C(=O)N1] | 2-(2,6-dioxopiperidin-3-yl)-6-(2-(6-(4-((1R,2S)-6-hydroxy-2- phenyl-1,2,3,4-tetrahydronaphthalen-1-yl)phenoxy)-2- azaspiro[3.3]heptan-2-yl)-2-oxoethyl)-6,7-dihydropyrrolo[3,4- f]isoindole-1,3(2H,5H)-dione |
| B277 | 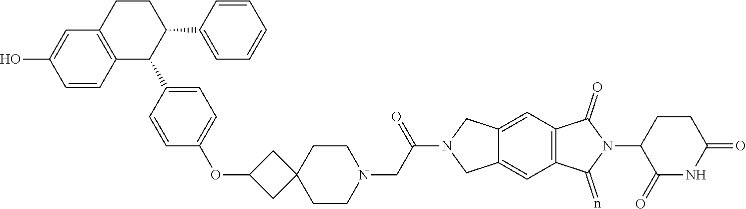 | 2-(2,6-dioxopiperidin-3-yl)-6-(2-(2-(4-((1R,2S)-6-hydroxy-2- phenyl-1,2,3,4-tetrahydronaphthalen-1-yl)phenoxy)-7- azaspiro[3.5]nonan-7-yl)acetyl)-6,7-dihydropyrrolo[3,4- f]isoindole-1,3(2H,5H)-dione |
| B278 | 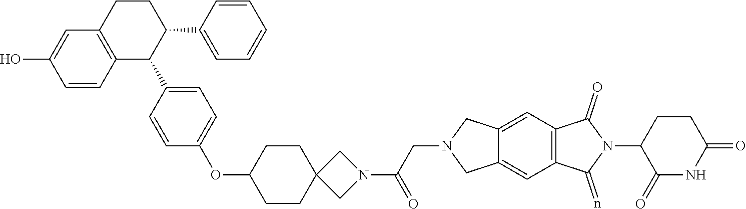 | 2-(2,6-dioxopiperidin-3-yl)-6-(2-(7-(4-((1R,2S)-6-hydroxy-2- phenyl-1,2,3,4-tetrahydronaphthalen-1-yl)phenoxy)-2- azaspiro[3.5]nonan-2-yl)-2-oxoethyl)-6,7-dihydropyrrolo[3,4- f]isoindole-1,3(2H,5H)-dione |
| B279 | 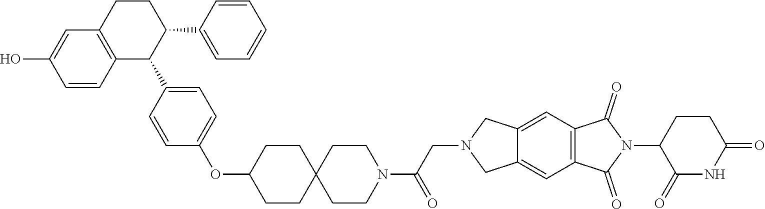 [SMILES: O=C1CCC(N2C(=O)c3cc4c(cc3C2=O)CN(CC(=O)N2CCC3(CCC(Oc5ccc([C@@H]6c7ccc(O)cc7CC[C@@H]6c6ccccc6)cc5)CC3)CC2)C4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)phenoxy)-3- azaspiro[5.5]undecan-3-yl)-2-oxoethyl)-6,7- dihydropyrrolo[3,4-f]isoindole-1,3(2H,5H)-dione |
| B280 | 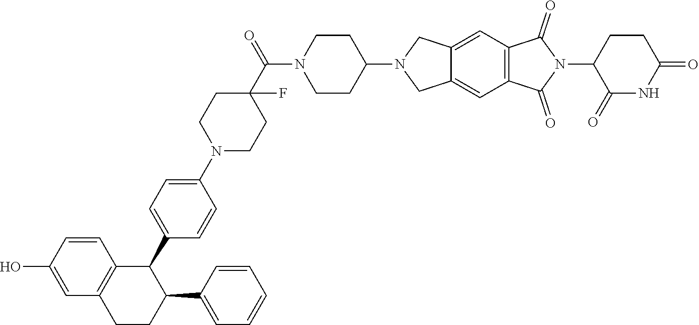 [SMILES: O=C1CCC(N2C(=O)c3cc4c(cc3C2=O)CN(C2CCN(C(=O)C3(F)CCN(c5ccc([C@@H]6c7ccc(O)cc7CC[C@@H]6c6ccccc6)cc5)CC3)CC2)C4)C(=O)N1] | 2-(2,6-dioxopiperidin-3-yl)-6-(1-(4-fluoro-1-(4-((1R,2S)-6- hydroxy-2-phenyl-1,2,3,4-tetrahydronaphthalen-1- yl)phenyl)piperidine-4-carbonyl)piperidin-4-yl)-6,7- dihydropyrrolo[3,4-f]isoindole-1,3(2H,5H)-dione |
| B281 | 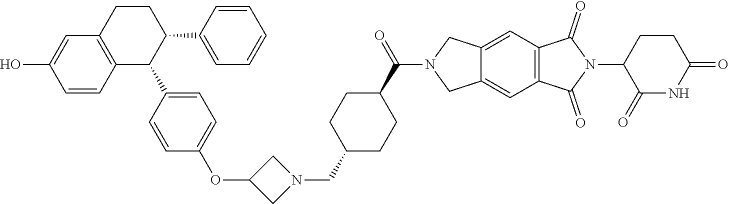 [SMILES: O=C1CCC(N2C(=O)c3cc4c(cc3C2=O)CN(C(=O)[C@H]2CC[C@H](CN3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-((1R,4r)-4-((3-(4-((1R,2S)-6- hydroxy-2-phenyl-1,2,3,4-tetrahydronaphthalen-1- yl)phenoxy)azetidin-1-yl)methyl)cyclohexane-1-carbonyl)-6,7- dihydropyrrolo[3,4-f]isoindole-1,3(2H,5H)-dione |
| B282 | 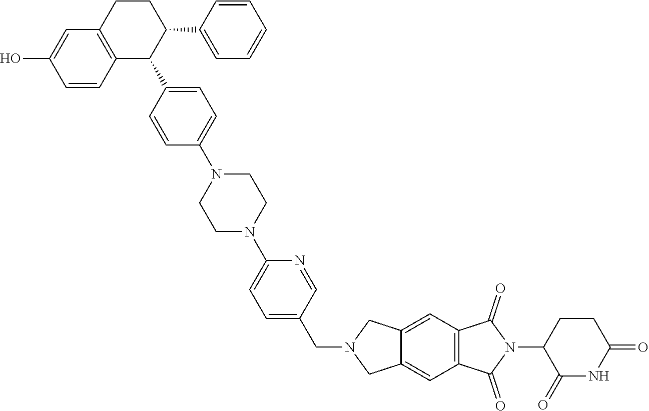 [SMILES: O=C1CCC(N2C(=O)c3cc4c(cc3C2=O)CN(Cc2ccc(N3CCN(c5ccc([C@@H]6c7ccc(O)cc7CC[C@@H]6c6ccccc6)cc5)CC3)nc2)C4)C(=O)N1] | 2-(2,6-dioxopiperidin-3-yl)-6-((6-(4-(4-((1R,2S)-6-hydroxy-2- phenyl-1,2,3,4-tetrahydronaphthalen-1-yl)phenyl)piperazin-1- yl)pyridin-3-yl)methyl)-6,7-dihydropyrrolo[3,4-f]isoindole- 1,3(2H,5H)-dione |
| B283 | 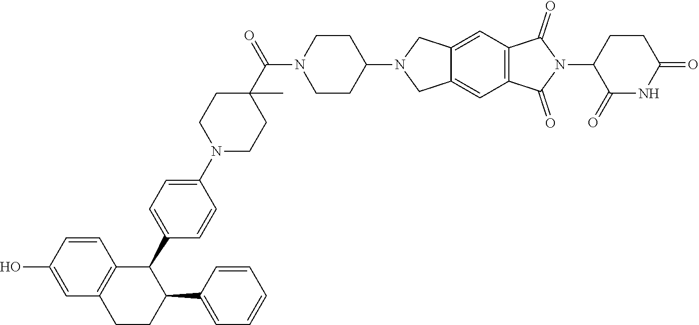 [SMILES: CC1(C(=O)N2CCC(N3Cc4cc5c(cc4C3)C(=O)N(C3CCC(=O)NC3=O)C5=O)CC2)CCN(c2ccc([C@@H]3c4ccc(O)cc4CC[C@@H]3c3ccccc3)cc2)CC1] | 2-(2,6-dioxopiperidin-3-yl)-6-(1-(1-(4-((1R,2S)-6-hydroxy-2- phenyl-1,2,3,4-tetrahydronaphthalen-1-yl)phenyl)-4- methylpiperidine-4-carbonyl)piperidin-4-yl)-6,7- dihydropyrrolo[3,4-f]isoindole-1,3(2H,5H)-dione |
| B284 | 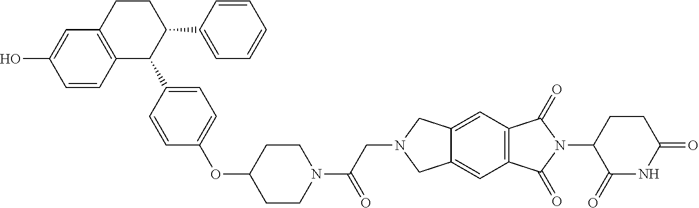 [SMILES: O=C1CCC(N2C(=O)c3cc4c(cc3C2=O)CN(CC(=O)N2CCC(Oc3ccc([C@@H]5c6ccc(O)cc6CC[C@@H]5c5ccccc5)cc3)CC2)C4)C(=O)N1] | 2-(2,6-dioxopiperidin-3-yl)-6-(2-(4-(4-((1R,2S)-6-hydroxy-2- phenyl-1,2,3,4-tetrahydronaphthalen-1-yl)phenoxy)piperidin-1- yl)-2-oxoethyl)-6,7-dihydropyrrolo[3,4-f]isoindole- 1,3(2H,5H)-dione |
| B285 | 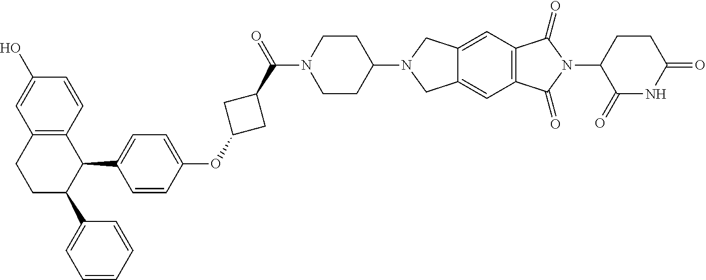 [SMILES: O=C1CCC(N2C(=O)c3cc4c(cc3C2=O)CN(C2CCN(C(=O)[C@H]3C[C@H](Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-(1-((1R,3r)-3-(4-((1R,2S)-6- hydroxy-2-phenyl-1,2,3,4-tetrahydronaphthalen-1- yl)phenoxy)cyclobutane-1-carbonyl)piperidin-4-yl)-6,7- dihydropyrrolo[3,4-f]isoindole-1,3(2H,5H)-dione |
| B286 | 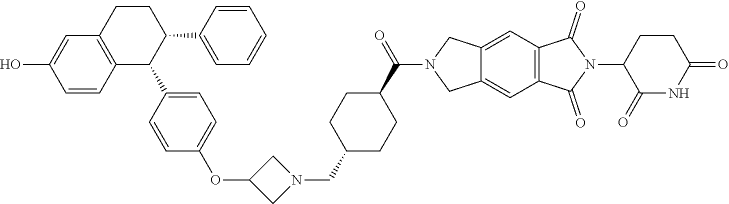 [SMILES: O=C1CCC(N2C(=O)c3cc4c(cc3C2=O)CN(C(=O)[C@H]2CC[C@H](CN3CC(Oc5ccc([C@@H]6c7ccc(O)cc7CC[C@@H]6c6ccccc6)cc5)C3)CC2)C4)C(=O)N1] | 3-(6-((1R,4r)-4-((3-(4-((1R,2S)-6-hydroxy-2-phenyl-1,2,3,4- tetrahydronaphthalen-1-yl)phenoxy)azetidin-1- yl)methyl)cyclohexane-1-carbonyl)-1-oxo-3,5,6,7- tetrahydropyrrolo[3,4-f]isoindol-2(1H)-yl)piperidine-2,6-dione |
| B287 | 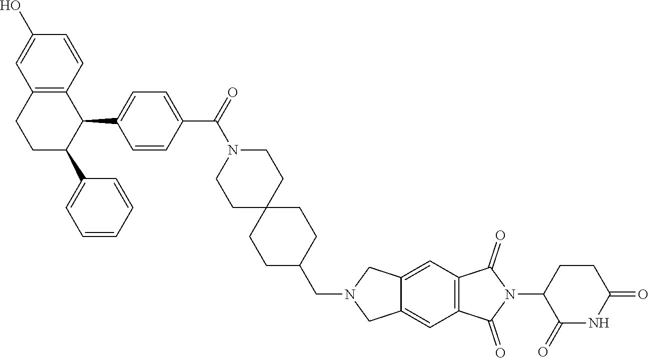 [SMILES: O=C1CCC(N2C(=O)c3cc4c(cc3C2=O)CN(CC2CCC3(CC2)CCN(C(=O)c2ccc([C@@H]5c6ccc(O)cc6CC[C@@H]5c5ccccc5)cc2)CC3)C4)C(=O)N1] | 2-(2,6-dioxopiperidin-3-yl)-6-((3-(4-((1R,2S)-6-hydroxy-2- phenyl-1,2,3,4-tetrahydronaphthalen-1-yl)benzoyl)-3- azaspiro[5.5]undecan-9-yl)methyl)-6,7-dihydropyrrolo[3,4- f]isoindole-1,3(2H,5H)-dione |
| B288 | 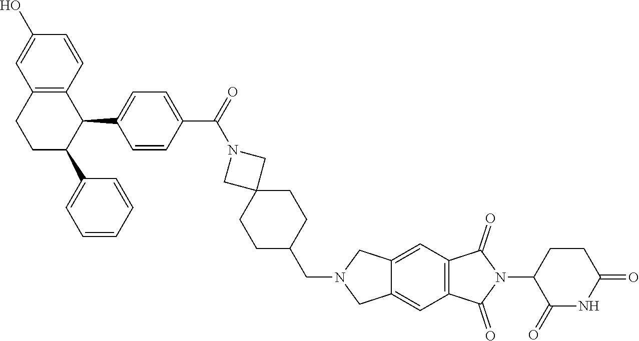 [SMILES: O=C1CCC(N2C(=O)c3cc4c(cc3C2=O)CN(CC2CCC3(CC2)CN(C(=O)c2ccc([C@@H]5c6ccc(O)cc6CC[C@@H]5c5ccccc5)cc2)C3)C4)C(=O)N1] | 2-(2,6-dioxopiperidin-3-yl)-6-((2-(4-((1R,2S)-6-hydroxy-2- phenyl-1,2,3,4-tetrahydronaphthalen-1-yl)benzoyl)-2- azaspiro[3.5]nonan-7-yl)methyl)-6,7-dihydropyrrolo[3,4- f]isoindole-1,3(2H,5H)-dione |
| B289 | 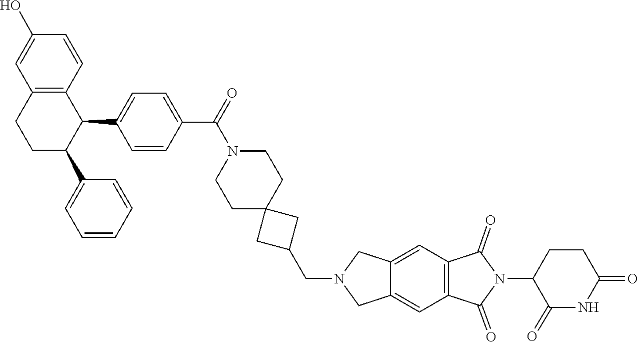 [SMILES: O=C1CCC(N2C(=O)c3cc4c(cc3C2=O)CN(CC2CC3(CCN(C(=O)c5ccc([C@@H]6c7ccc(O)cc7CC[C@@H]6c6ccccc6)cc5)CC3)C2)C4)C(=O)N1] | 2-(2,6-dioxopiperidin-3-yl)-6-((7-(4-((1R,2S)-6-hydroxy-2- phenyl-1,2,3,4-tetrahydronaphthalen-1-yl)benzoyl)-7- azaspiro[3.5]nonan-2-yl)methyl)-6,7-dihydropyrrolo[3,4- f]isoindole-1,3(2H,5H)-dione |
| B290 | 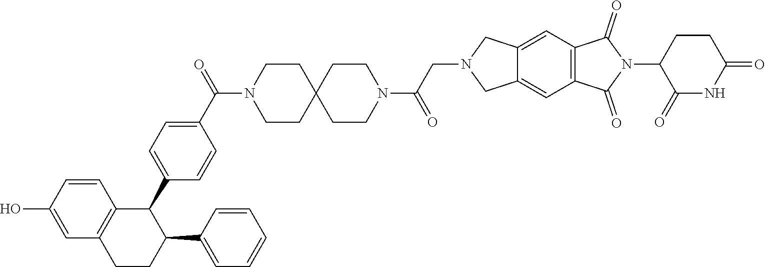 [SMILES: O=C1CCC(N2C(=O)c3cc4c(cc3C2=O)CN(CC(=O)N2CCC3(CC2)CCN(C(=O)c2ccc([C@@H]5c6ccc(O)cc6CC[C@@H]5c5ccccc5)cc2)CC3)C4)C(=O)N1] | 2-(2,6-dioxopiperidin-3-yl)-6-(2-(9-(4-((1R,2S)-6-hydroxy-2- phenyl-1,2,3,4-tetrahydronaphthalen-1-yl)benzoyl)-3,9- diazaspiro[5.5]undecan-3-yl)-2-oxoethyl)-6,7- dihydropyrrolo[3,4-f]isoindole-1,3(2H,5H)-dione |
| B291 | 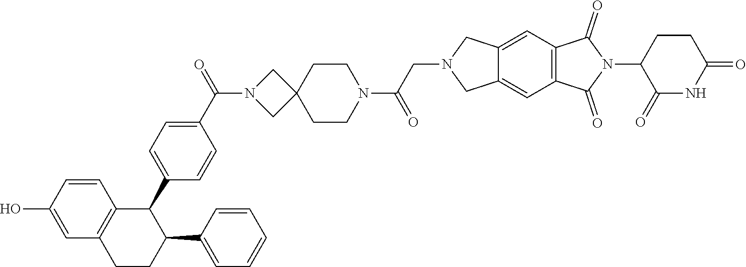 [SMILES: O=C1CCC(N2C(=O)c3cc4c(cc3C2=O)CN(CC(=O)N2CCC3(CC2)CN(C(=O)c2ccc([C@@H]5c6ccc(O)cc6CC[C@@H]5c5ccccc5)cc2)C3)C4)C(=O)N1] | 2-(2,6-dioxopiperidin-3-yl)-6-(2-(2-(4-((1R,2S)-6-hydroxy-2- phenyl-1,2,3,4-tetrahydronaphthalen-1-yl)benzoyl)-2,7- diazaspiro[3.5]nonan-7-yl)-2-oxoethyl)-6,7- dihydropyrrolo[3,4-f]isoindole-1,3(2H,5H)-dione |
| B292 | 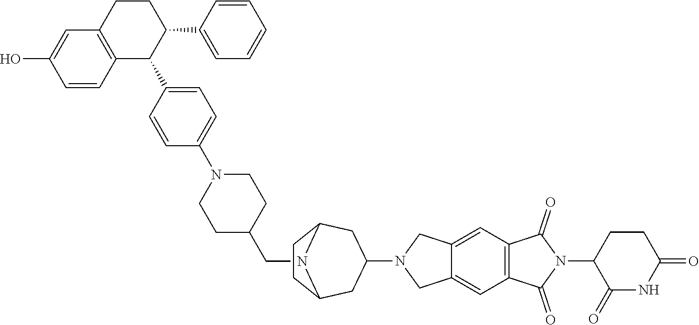 [SMILES: O=C1CCC(N2C(=O)c3cc4c(cc3C2=O)CN(C2CC3CCC(C2)N3CC2CCN(c3ccc([C@@H]5c6ccc(O)cc6CC[C@@H]5c5ccccc5)cc3)CC2)C4)C(=O)N1] | 2-(2,6-dioxopiperidin-3-yl)-6-(8-((1-(4-((1R,2S)-6-hydroxy-2- phenyl-1,2,3,4-tetrahydronaphthalen-1-yl)phenyl)piperidin-4- yl)methyl)-8-azabicyclo[3.2.1]octan-3-yl)-6,7- dihydropyrrolo[3,4-f]isoindole-1,3(2H,5H)-dione |
| B293 | 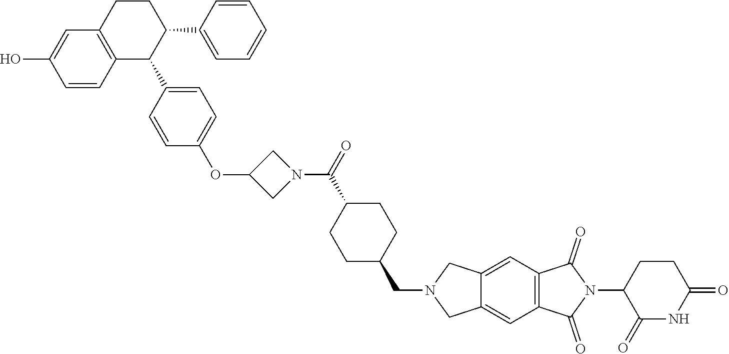 [SMILES: O=C1CCC(N2C(=O)c3cc4c(cc3C2=O)CN(C[C@H]2CC[C@H](C(=O)N3CC(Oc5ccc([C@@H]6c7ccc(O)cc7CC[C@@H]6c6ccccc6)cc5)C3)CC2)C4)C(=O)N1] | 2-(2,6-dioxopiperidin-3-yl)-6-(((1R,4r)-4-(3-(4-((1R,2S)-6- hydroxy-2-phenyl-1,2,3,4-tetrahydronaphthalen-1- yl)phenoxy)azetidine-1-carbonyl)cyclohexyl)methyl)-6,7- dihydropyrrolo[3,4-f]isoindole-1,3(2H,5H)-dione |
| B294 | 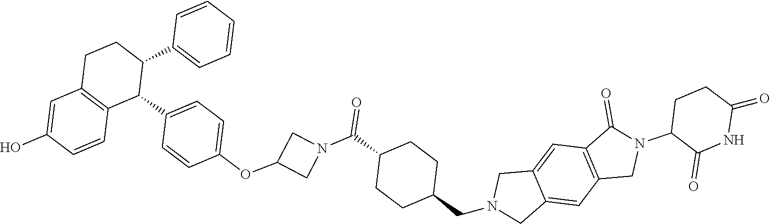 [SMILES: O=C1CCC(N2Cc3cc4c(cc3C2=O)CN(C[C@H]2CC[C@H](C(=O)N3CC(Oc5ccc([C@@H]6c7ccc(O)cc7CC[C@@H]6c6ccccc6)cc5)C3)CC2)C4)C(=O)N1] | 3-(6-(((1R,4r)-4-(3-(4-((1R,2S)-6-hydroxy-2-phenyl-1,2,3,4- tetrahydronaphthalen-1-yl)phenoxy)azetidine-1- carbonyl)cyclohexyl)methyl)-1-oxo-3,5,6,7- tetrahydropyrrolo[3,4-f]isoindol-2(1H)-yl)piperidine-2,6-dione |
| B296 | 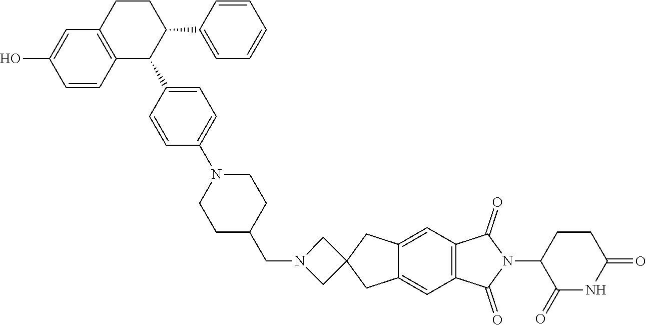 [SMILES: O=C1CCC(N2C(=O)c3cc4c(cc3C2=O)CC2(C4)CN(CC3CCN(c4ccc([C@@H]5c6ccc(O)cc6CC[C@@H]5c5ccccc5)cc4)CC3)C2)C(=O)N1] | 2′-(2,6-dioxopiperidin-3-yl)-1-((1-(4-((1R,2S)-6-hydroxy-2- phenyl-1,2,3,4-tetrahydronaphthalen-1-yl)phenyl)piperidin-4- yl)methyl)-5′,7′-dihydro-1′H-spiro[azetidine-3,6′- cyclopenta[f]isoindole]-1′,3′(2'H)-dione |
| B297 | 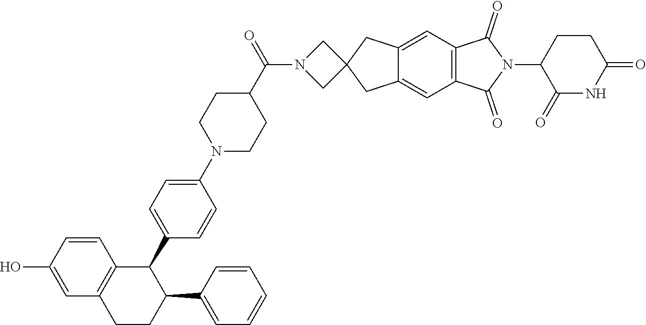 [SMILES: O=C1CCC(N2C(=O)c3cc4c(cc3C2=O)CC2(C4)CN(C(=O)C3CCN(c4ccc([C@@H]5c6ccc(O)cc6CC[C@@H]5c5ccccc5)cc4)CC3)C2)C(=O)N1] | 2′-(2,6-dioxopiperidin-3-yl)-1-(1-(4-((1R,2S)-6-hydroxy-2- phenyl-1,2,3,4-tetrahydronaphthalen-1-yl)phenyl)piperidine-4- carbonyl)-5′,7′-dihydro-1′H-spiro[azetidine-3,6′- cyclopenta[f]isoindole]-1′,3′(2'H)-dione |
| B298 | 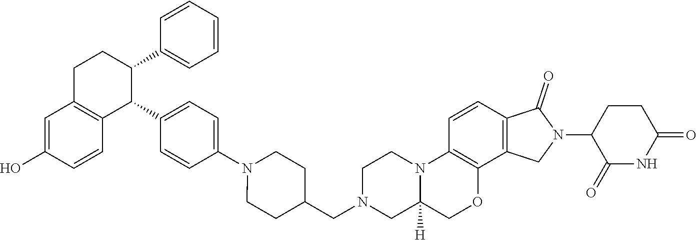 [SMILES: O=C1CCC(N2Cc3c(ccc4c3OC[C@H]3CN(CC5CCN(c6ccc([C@@H]7c8ccc(O)cc8CC[C@@H]7c7ccccc7)cc6)CC5)CCN43)C2=O)C(=O)N1] | 3-((R)-7-((1-(4-((1R,2S)-6-hydroxy-2-phenyl-1,2,3,4- tetrahydronaphthalen-1-yl)phenyl)piperidin-4-yl)methyl)-1- oxo-1,3,5,5a,6,7,8,9-octahydro-2H- pyrazino[1′,2′:4,5][1,4]oxazino[2,3-e]isoindol-2-yl)piperidine- 2,6-dione |
| B299 | 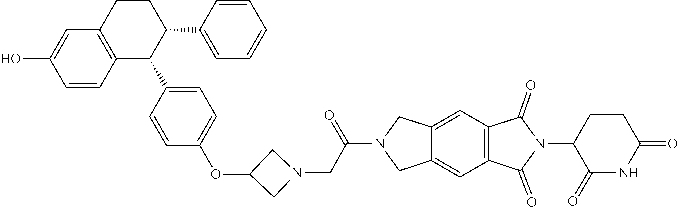 [SMILES: O=C1CCC(N2C(=O)c3cc4c(cc3C2=O)CN(C(=O)CN2CC(Oc3ccc([C@@H]5c6ccc(O)cc6CC[C@@H]5c5ccccc5)cc3)C2)C4)C(=O)N1] | 2′-(2,6-dioxopiperidin-3-yl)-1-(2-(3-(4-((1R,2S)-6-hydroxy-2- phenyl-1,2,3,4-tetrahydronaphthalen-1-yl)phenoxy)azetidin-1- yl)acetyl)-5′,7′-dihydro-1'H-spiro[azetidine-3,6′- cyclopenta[f]isoindole]-1′,3′(2'H)-dione |
| B300 | 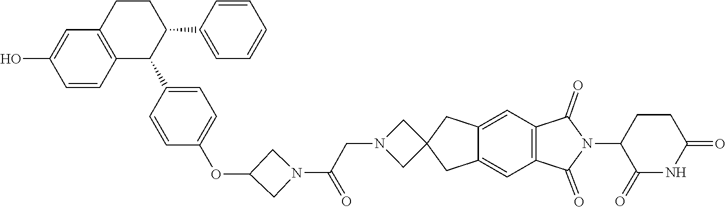 [SMILES: O=C1CCC(N2C(=O)c3cc4c(cc3C2=O)CC2(C4)CN(CC(=O)N3CC(Oc4ccc([C@@H]5c6ccc(O)cc6CC[C@@H]5c5ccccc5)cc4)C3)C2)C(=O)N1] | 2′-(2,6-dioxopiperidin-3-yl)-1-(2-(3-(4-((1R,2S)-6-hydroxy-2- phenyl-1,2,3,4-tetrahydronaphthalen-1-yl)phenoxy)azetidin-1- yl)-2-oxoethyl)-5′,7′-dihydro-1'H-spiro[azetidine-3,6′- cyclopenta[f]isoindole]-1′,3′(2′H)-dione |
| B301 | 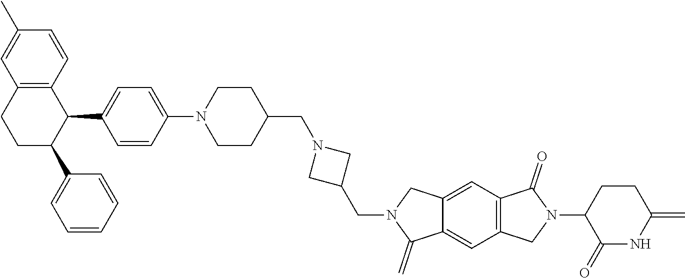 [SMILES: C=C1CCC(N2Cc3cc4c(cc3C2=O)CN(CC2CN(CC3CCN(c5ccc([C@@H]6c7ccc(C)cc7CC[C@@H]6c6ccccc6)cc5)CC3)C2)C4=C)C(=O)N1] | 2-(2,6-dioxopiperidin-3-yl)-6-((1-((1-(4-((1R,2S)-6-hydroxy-2- phenyl-1,2,3,4-tetrahydronaphthalen-1-yl)phenyl)piperidin-4- yl)methyl)azetidin-3-yl)methyl)-2,3,6,7-tetrahydropyrrolo[3,4- f]isoindole-1,5-dione |
| B302 | 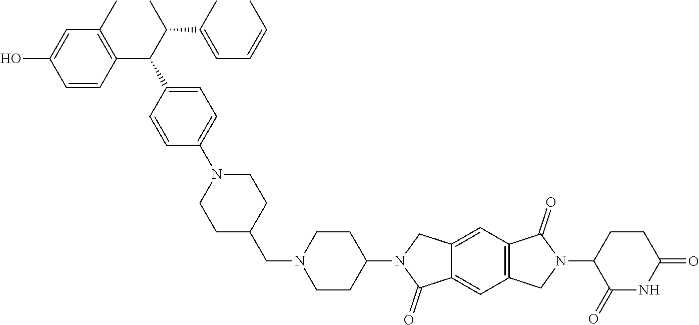 [SMILES: C/C=C\C=C(/C)[C@@H](C)[C@H](c1ccc(N2CCC(CN3CCC(N4Cc5cc6c(cc5C4=O)CN(C4CCC(=O)NC4=O)C6=O)CC3)CC2)cc1)c1ccc(O)cc1C] | 2-(2,6-dioxopiperidin-3-yl)-6-(1-((1-(4-((1R,2S)-6-hydroxy-2- phenyl-1,2,3,4-tetrahydronaphthalen-1-yl)phenyl)piperidin-4- yl)methyl)azetidin-3-yl)-2,3,6,7-tetrahydropyrrolo[3,4- f]isoindole-1,5-dione |
| B303 | 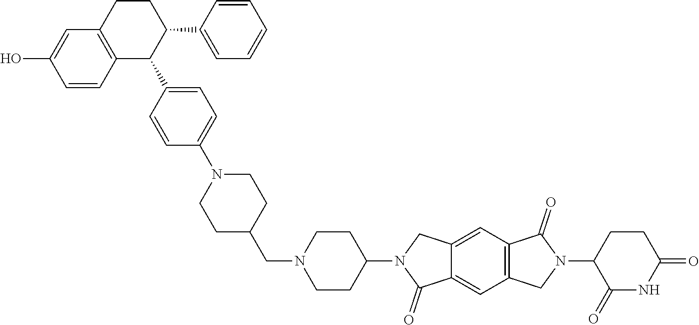 [SMILES: O=C1CCC(N2Cc3cc4c(cc3C2=O)CN(C2CCN(CC3CCN(c5ccc([C@@H]6c7ccc(O)cc7CC[C@@H]6c6ccccc6)cc5)CC3)CC2)C4=O)C(=O)N1] | 2-(2,6-dioxopiperidin-3-yl)-6-(1-((1-(4-((1R,2S)-6-hydroxy-2- phenyl-1,2,3,4-tetrahydronaphthalen-1-yl)phenyl)piperidin-4- yl)methyl)piperidin-4-yl)-2,3,6,7-tetrahydropyrrolo[3,4- f]isoindole-1,5-dione |
| B304 | 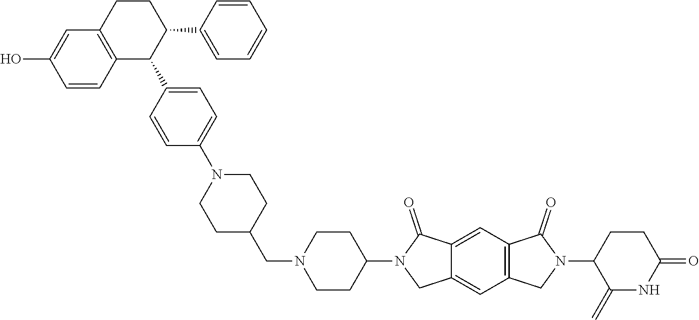 [SMILES: C=C1NC(=O)CCC1N1Cc2cc3c(cc2C1=O)C(=O)N(C1CCN(CC2CCN(c4ccc([C@@H]5c6ccc(O)cc6CC[C@@H]5c5ccccc5)cc4)CC2)CC1)C3] | 2-(2,6-dioxopiperidin-3-yl)-6-(1-((1-(4-((1R,2S)-6-hydroxy-2- phenyl-1,2,3,4-tctrahydronaphthalcn-1-yl)phenyl)pipcridin-4- yl)methyl)piperidin-4-yl)-2,3,5,6-tetrahydropyrrolo[3,4- f]isoindole-1,7-dione |
| B305 | 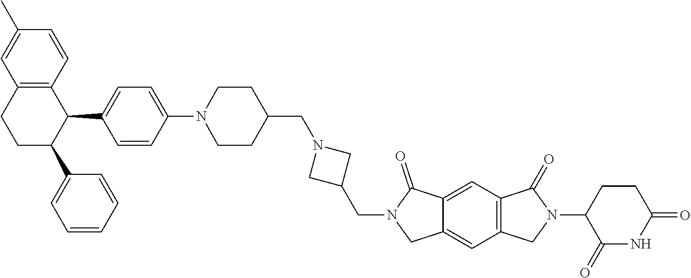 [SMILES: Cc1ccc2c(c1)CC[C@H](c1ccccc1)[C@@H]2c1ccc(N2CCC(CN3CC(CN4Cc5cc6c(cc5C4=O)C(=O)N(C4CCC(=O)NC4=O)C6)C3)CC2)cc1] | 2-(2,6-dioxopiperidin-3-yl)-6-((1-((1-(4-((1R,2S)-6-hydroxy-2- phenyl-1,2,3,4-tetrahydronaphthalen-1-yl)phenyl)piperidin-4- yl)mcthyl)azctidin-3-yl)methyl)-2,3,5,6-tctrahydropyrrolo[3,4- f]isoindole-1,7-dione |
| B323 | 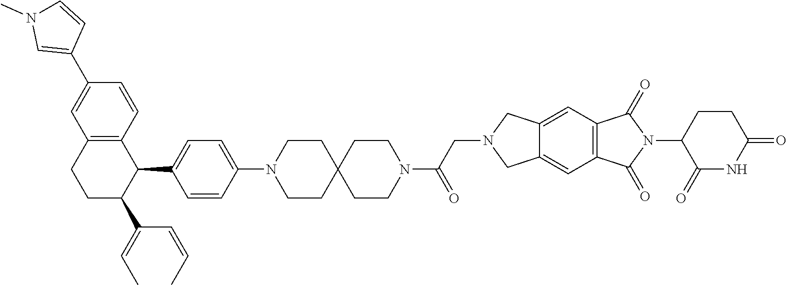 [SMILES: C/C=C\C(=C/C)[C@H]1CCc2cc(-c3ccn(C)c3)ccc2[C@H]1c1ccc(N2CCC3(CCN(C(=O)CN4Cc5cc6c(cc5C4)C(=O)N(C4CCC(=O)NC4=O)C6=O)CC3)CC2)cc1] | 2-(2,6-dioxopiperidin-3-yl)-6-(2-(9-(4-((1R,2S)-6-(1-methyl- 1H-pyrazol-4-yl)-2-phenyl-1,2,3,4-tetrahydronaphthalen-1- yl)phenyl)-3,9-diazaspiro[5.5]undecan-3-yl)-2-oxoethyl)-6,7- dihydropyrrolo[3,4-f]isoindole-1,3(2H,5H)-dione |
| B323 | 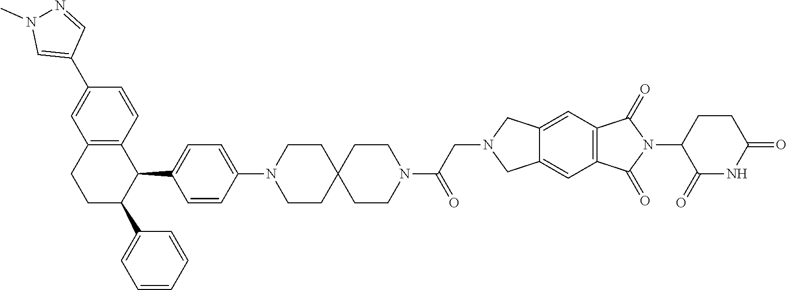 [SMILES: Cn1cc(-c2ccc3c(c2)CC[C@H](c2ccccc2)[C@@H]3c2ccc(N3CCC4(CCN(C(=O)CN5Cc6cc7c(cc6C5)C(=O)N(C5CCC(=O)NC5=O)C7=O)CC4)CC3)cc2)cn1] | 2-(2,6-dioxopiperidin-3-yl)-6-(2-(9-(4-((1R,2S)-6-(1-methyl- 1H-pyrazol-4-yl)-2-phenyl-1,2,3,4-tetrahydronaphthalen-1- yl)phenyl)-3,9-diazaspiro[5.5]undecan-3-yl)-2-oxocthyl)-6,7- dihydropyrrolo[3,4-f]isoindole-1,3(2H,5H)-dione |
| B324 | 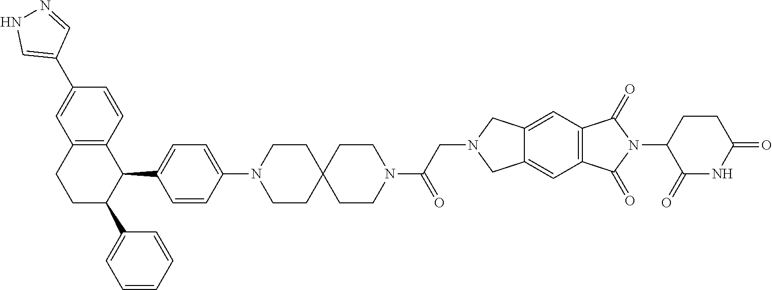 [SMILES: O=C1CCC(N2C(=O)c3cc4c(cc3C2=O)CN(CC(=O)N2CCC3(CC2)CCN(c2ccc([C@@H]5c6ccc(-c7cn[nH]c7)cc6CC[C@@H]5c5ccccc5)cc2)CC3)C4)C(=O)N1] | 2-(2,6-dioxopiperidin-3-yl)-6-(2-oxo-2-(9-(4-((1R,2S)-2- phenyl-6-(1H-pyrazol-4-yl)-1,2,3,4-tetrahydronaphthalen-1- yl)phenyl)-3,9-diazaspiro[5.5]undecan-3-yl)ethyl)-6,7- dihydropyrrolo[3,4-f]isoindole-1,3(2H,5H)-dione |
| B325 | 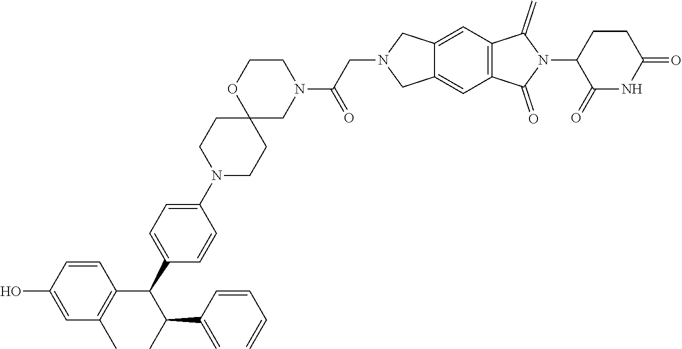 [SMILES: C=C/C=C\C(=C)[C@@H](C)[C@H](c1ccc(N2CCC3(CC2)CN(C(=O)CN2Cc4cc5c(cc4C2)C(=O)N(C2CCC(=O)NC2=O)C5=C)CCO3)cc1)c1ccc(O)cc1C] | 2-(2,6-dioxopiperidin-3-yl)-6-(2-(9-(4-((1R,2S)-6-hydroxy-2- phenyl-1,2,3,4-tetrahydronaphthalen-1-yl)phenyl)-1-oxa-4,9- diazaspiro[5.5]undecan-4-yl)-2-oxoethyl)-6,7- dihydropyrrolo[3,4-f]isoindole-1,3(2H,5H)-dione |
| B338 | 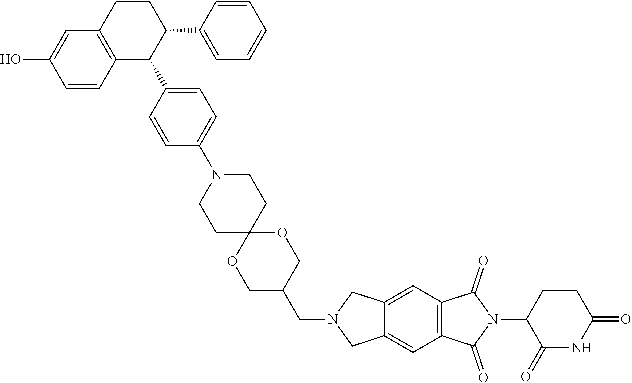 [SMILES: O=C1CCC(N2C(=O)c3cc4c(cc3C2=O)CN(CC2COC3(CCN(c5ccc([C@@H]6c7ccc(O)cc7CC[C@@H]6c6ccccc6)cc5)CC3)OC2)C4)C(=O)N1] | 2-(2,6-dioxopiperidin-3-yl)-6-((9-(4-((1R,2S)-6-hydroxy-2- phenyl-1,2,3,4-tetrahydronaphthalen-1-yl)phenyl)-1,5-dioxa-9- azaspiro[5.5]undecan-3-yl)methyl)-6,7-dihydropyrrolo[3,4- f]isoindole-1,3(2H,5H)-dione |
| B352 | 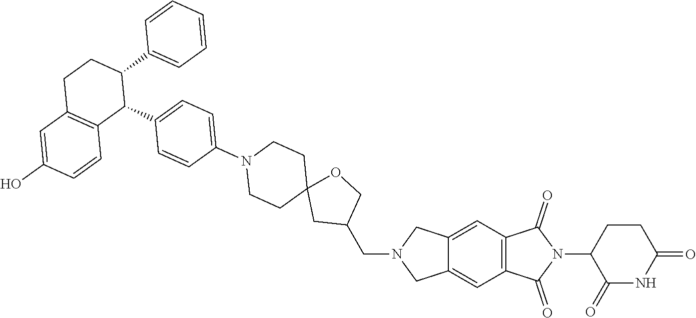 [SMILES: O=C1CCC(N2C(=O)c3cc4c(cc3C2=O)CN(CC2COC3(CCN(c5ccc([C@@H]6c7ccc(O)cc7CC[C@@H]6c6ccccc6)cc5)CC3)C2)C4)C(=O)N1] | 3-(6-((8-(4-((1R,2S)-6-hydroxy-2-phenyl-1,2,3,4- tetrahydronaphthalcn-1-yl)phcnyl)-1-oxa-8- azaspiro[4.5]decan-3-yl)methyl)-1-oxo-3,5,6,7- tetrahydropyrrolo[3,4-f]isoindol-2(1H)-yl)piperidine-2,6-dione |
| B355 | 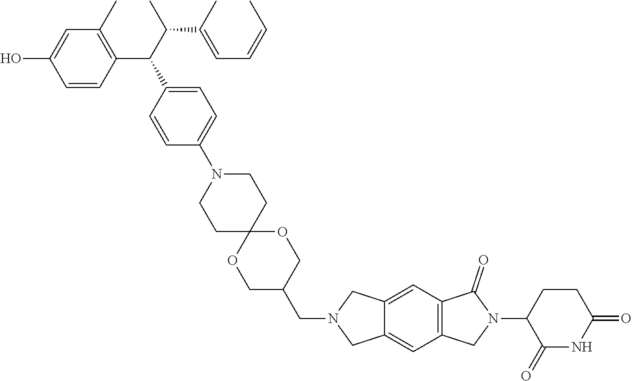 [SMILES: C/C=C\C=C(/C)[C@@H](C)[C@H](c1ccc(N2CCC3(CC2)OCC(CN2Cc4cc5c(cc4C2)C(=O)N(C2CCC(=O)NC2=O)C5)CO3)cc1)c1ccc(O)cc1C] | 3-(6-((9-(4-((1R,2S)-6-hydroxy-2-phenyl-1,2,3,4- tetrahydronaphthalen-1-yl)phenyl)-1,5-dioxa-9- azaspiro[5.5]undecan-3-yl)methyl)-1-oxo-3,5,6,7- tetrahydropyrrolo[3,4-f]isoindol-2(1H)-yl)piperidine-2,6-dione |
| B389 | 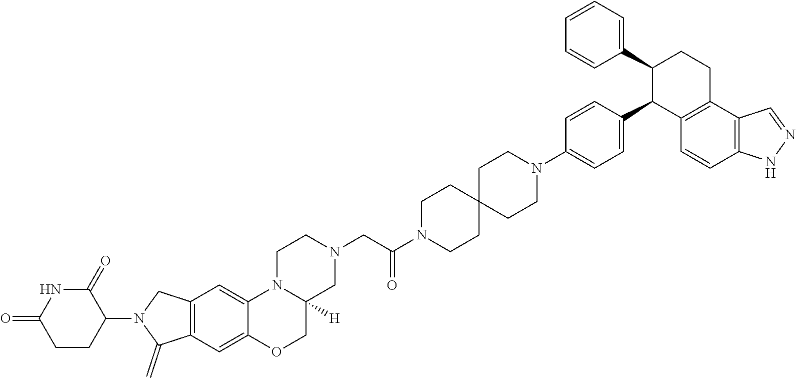 [SMILES: C=C1c2cc3c(cc2CN1C1CCC(=O)NC1=O)N1CCN(CC(=O)N2CCC4(CC2)CCN(c2ccc([C@@H]5c6ccc7[nH]ncc7c6CC[C@@H]5c5ccccc5)cc2)CC4)C[C@H]1CO3] | 3-((S)-8-oxo-3-(2-oxo-2-(9-(4-((6R,7S)-7-phenyl-6,7,8,9- tetrahydro-3H-benzo[e]indazol-6-yl)phenyl)-3,9- diazaspiro[5.5]undecan-3-yl)ethyl)-1,2,3,4,4a,5,8,10- octahydro-9H-pyrazino[1′,2′:4,5][1,4]oxazino[2,3-f]isoindol-9- yl)piperidine-2,6-dione |
| B390 | 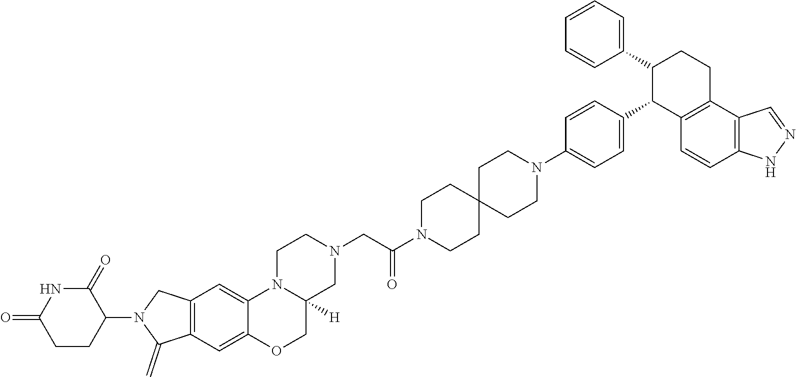 | 3-((S)-8-oxo-3-(2-oxo-2-(9-(4-((6S,7R)-7-phenyl-6,7,8,9- tetrahydro-3H-benzo[e]indazol-6-yl)phenyl)-3,9- diazaspiro[5.5]undecan-3-yl)ethyl)-1,2,3,4,4a,5,8,10- octahydro-9H-pyrazino[1′,2′:4,5][1,4]oxazino[2,3-f]isoindol-9- yl)piperidine-2,6-dione |

The compounds of the present disclosure may possess advantageous characteristics, as compared to known compounds, such as known estrogen receptor degraders. For example, the compounds of the present disclosure may display more potent estrogen receptor activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In certain embodiments, a compound of the present disclosure (e.g., a compound of any of the formulae or any individual compounds disclosed herein) is a pharmaceutically acceptable salt. In certain embodiments, a compound of the present disclosure (e.g., a compound of any of the formulae or any individual compounds disclosed herein) is a solvate. In certain embodiments, a compound of the present disclosure (e.g., a compound of any of the formulae or any individual compounds disclosed herein) is a hydrate.

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl) 4, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof arc contemplated and are within the scope of the present disclosure.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers (Stereoisomers, Geometric Isomer, Tautomer, Etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)- isomers respectively). A chiral compound can exist as either individual enantiomer or as a mixture thereof. A mixture containing equal proportions of the enantiomers is termed a “racemic mixture”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure®-compound” refers to at least about 95% by weight®-compound and at most about 5% by weight(S) -compound, at least about 99% by weight (R)-compound and at most about 1% by weight(S)-compound, or at least about 99.9% by weight (R)-compound and at most about 0.1% by weight(S) -compound. In certain embodiments, the weights are based upon total weight of compound.

As used herein and unless otherwise indicated, the term “enantiomerically pure(S)-compound” refers to at least about 95% by weight(S)-compound and at most about 5% by weight (R)-compound, at least about 99% by weight(S)-compound and at most about 1% by weight (R)-compound or at least about 99.9% by weight(S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight(S)-compound, by total weight of the compound. For example, a pharmaceutical composition comprising enantiomerically pure(S)-compound can comprise, for example, about 90% excipient and about 10% enantiomerically pure(S)-compound. In certain embodiments, the enantiomerically pure(S)-compound in such compositions can, for example, comprise, at least about 95% by weight(S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers arc well-known in the art.

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci-and nitro-forms of phenylnitromethane, that arc likewise formed by treatment with acid or base. Tautomcric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Method

The compounds of the present disclosure can generally be prepared by first preparing pools of intermediates, including a pool of cereblon ligands, a pool of linkers, and a pool of inhibitors, as detailed in the Example section, then followed by subsequent reactions to connect a linker to an inhibitor and a cereblon ligand via metal-catalyzed coupling reactions and reductive amination. Large pool of compounds can be prepared by selecting different combinations of cereblon ligands, linkers, and inhibitors from each pool, General synthetic routes for preparing inhibitor-linker conjugate via metal-catalyzed coupling reactions, which is further coupled to cerebon ligand via reductive amination, are summarize below.

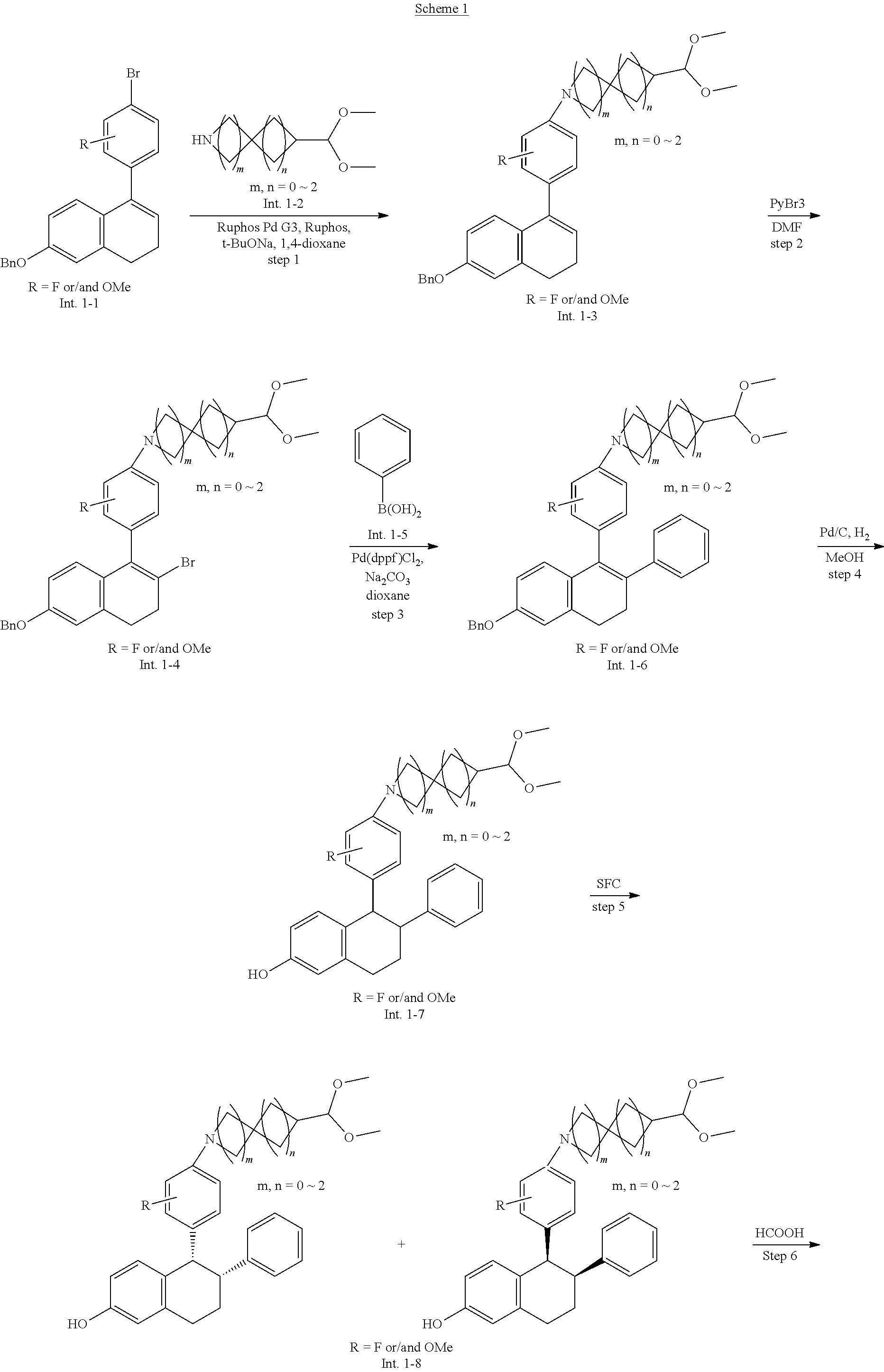

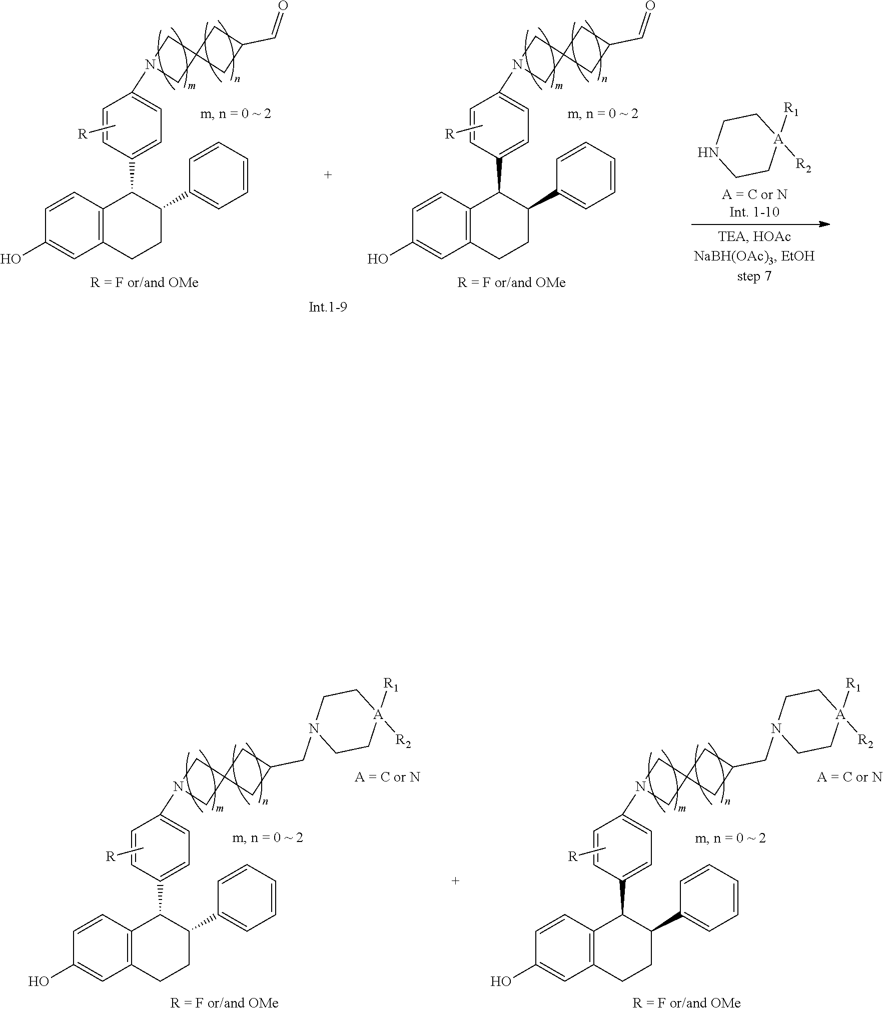

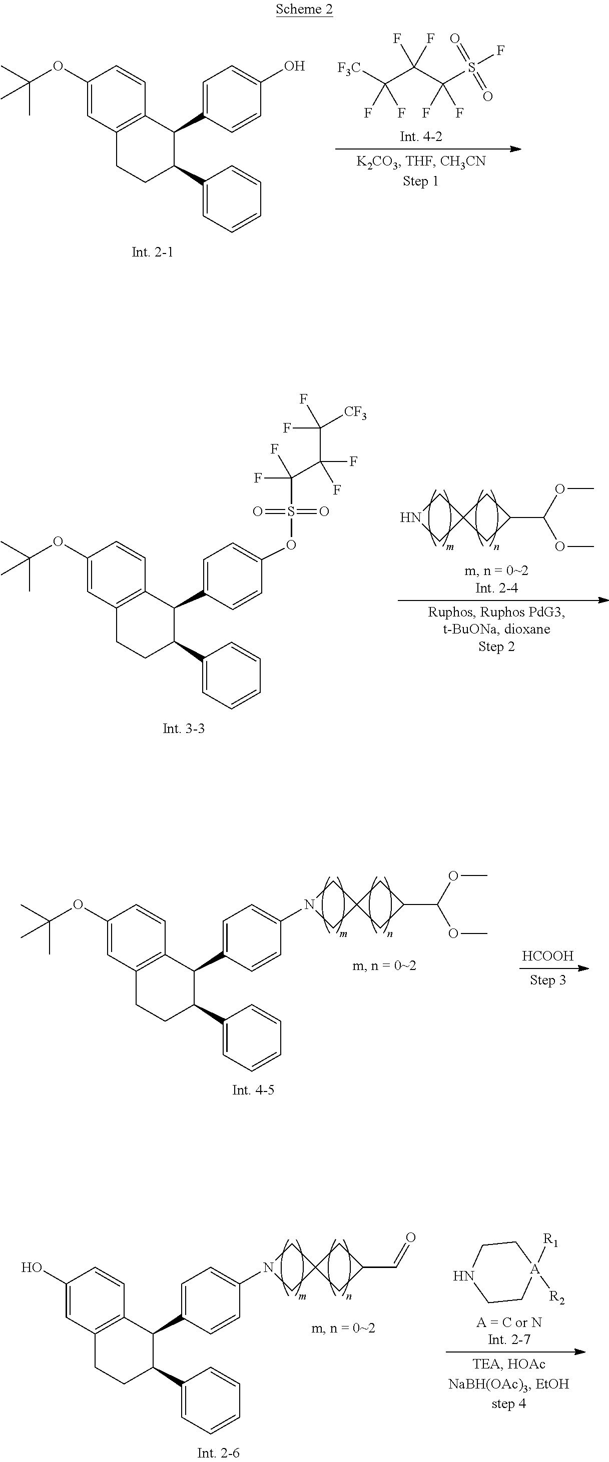

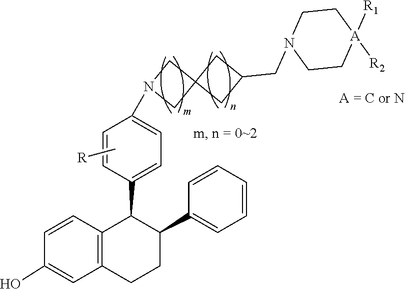

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall. U.K.), Chem Service Inc. (West Chester, PA), Crescent Chemical Co. (Hauppauge. NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windbam, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif, 1972; T. L, Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example. Fuhrhop. J, and Penzlin G. “Organic Synthesis: Concepts, Methods, Starting Materials”, Second, Revised and Enlarged Edition (1994) John Wiley & Sons ISBN: 3-527-29074-5; Hoffman. R.V. “Organic Chemistry. An Intermediate Text” (1996)Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons. T. W, G. “Organic Chemistry” 7th Edition (2000) John Wiley & Sons, ISBN: 0-471-19095-0); Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X. in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C, G. Wermuth “Handbook of Pharmaceutical Salts”. Verlag Helvetica Chimica Acta, Zurich, 2002.

### Analytical Methods, Materials, and Instrumentation

Unless otherwise noted, reagents and solvents are used as received from commercial suppliers. Proton nuclear magnetic resonance (NMR) spectra were obtained on either Bruker or Varian spectrometers at 400 MHz. Spectra are given in ppm (δ) and coupling constants. J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low-resolution mass spectral data were measured using Agilent 1260-6125B LCMS system (with Diode Array Detector, and Agilent G6125BA Mass spectrometer) or using Waters Aequity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Aequity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present application can be assessed with methods and assays known in the art.

The CRBN-DDB1 binding potency of the present disclosure is determined using HTRF assay technology (Perkin Elmer). Compounds are serially diluted and are transferred multi-well plate. The reaction is conducted with addition of His-tagged (e.g., CRBN+DDB-DLS7+CXU4) followed by addition of 60 nM fluorescent probe (e.g., Cy5-labeled Thalidomide), and MAb Anti-6HIS Tb cryptate Gold in the assay buffer. After one hour incubation at room temperature, the HTRF signals are read, e.g., on Envision reader (Perkin Elemer).

ERα degradative activity of compounds can be assessed in MCF-7 and T47D Cells. MCF-7 and T47D cell are seeded and are subsequently treated with the compounds at certain concentrations (e.g., 0.02 to 300 nM). DMSO can be used as vehicle control. Cells are fixed and are blocked with Intercept (PBS) Blocking Buffer (e.g., Li-COR, Odyssey Blocking Buffer), and are stained with ER (e.g., 1:500, Cell signaling) primary antibody for overnight in a cold room (e.g., 4° C.). Secondary Antibody (e.g., IRDye 800CW Goat anti-Rabbit IgG) and CellTag 700 Stain are added in Intercept (PBS) Blocking Buffer. Finally, cell plate is placed in incubator to dry. Image and signal were captured on Odyssey® DLx Imaging System.

An in vitro assay can be accomplished by an MCF-7 and T47D Cell Titer Glo (CTG) assay. MCF-7 and T47D cell (From HDB) are cultured in a multi-well white plate with phenol red-free RPMI1640+10% CS-FBS+1% P/S medium (e.g., at 1,000cells/well). On day 0, cells were treated with compound at certain concentrations (e.g., 0.5 to 10000 nM) (DMSO and Staurosporine as control). On day 0 and day 6 Cell Titer Glo reagent is added and read on En Vision after 30 min incubation for data generation.

For in-cell western blot analysis, cells are seeded in multi-well plates (e.g., at 40,000 or 10,000 cells/well). Diluted compounds at certain concentration are added (final 0.5% DMSO) and cells are incubated for certain period of time (e.g., 16 hours). Formaldehyde (e.g., PBS:FA=9:1) is added and followed by washing with PBS. The cells are blocked with Licor blocking buffer (Li-Cor). The relative ER percentage in treated cells is obtained by comparing the values of treated wells to those in untreated and DMSO-treated wells as 100%.

For western blot analysis, cells that are treated with the compounds are lysed in Radioimmunoprecipitation Assay Protein Lysis and Extraction Buffer (e.g., 25 mmol/L Tris.HCl, pH 7.6, 150 mmol/I. NaCl, 1% Nonidet P-40, 1% sodium deoxycholate, and 0.1% sodium dodecyl sulfate) containing proteinase inhibitor cocktail. Equal amounts of total protein are electrophoresed through 10% SDS-polyacrylamide gels after determination of protein concentration by BCA assay. The separated protein bands are transferred onto PVDF membranes and blotted against different antibodies. The blots are scanned, and the band intensities are quantified (e.g., by using GelQuant.NET software provided by biochemlabsolutions.com). The relative mean intensity of target proteins is expressed after normalization to the intensity of glyceraldehyde-3-phosphate dehydrogenase bands.

For the cell growth assay, cells are seeded at certain concentrations (e.g., at 1500/well) in multi-well plates overnight. Cells are subsequently treated with the compounds. A certain period of time (e.g., 4 days) after the compound treatment, 10% WST-8 reagent is added to the culture medium and incubated under certain condiction (e.g., in a CO₂ incubator at 37° C. for 2.5 hours). The absorbance is measured on each sample using a microplate reader at certain wavelength (e.g., 450 nm). The relative absorbance is calculated against the vehicle control from three individually repeats.

For in vivo pharmacodynamic and efficacy studies, breast cancer cell line xenografts are developed as follows: mice are given 17ß-Estradiol in drinking water for a certain period of time. A certain number (e.g., five million) of cells in 50% Matrigel are injected subcutaneously into SCID mice to induce tumor formation. When tumors reach a certain size (e.g., 100-400 mm³), mice are treated with vehicle control (e.g., 5% DMSO, 10% solutol, 85% water) or the compound, and sacrificed at various time points. Tumor tissue is harvested for analysis. Tumor sizes and animal weights are measured 2-3 times per week. Tumor volume (mm³)=(length×width2)/2. Tumor growth inhibition is calculated using TGI (%)=(Vc−Vt)/(Vc−Vo)× 100, where Vc, Vt are the median of control and treated groups at the end of the study and Vo at the start.

### Methods of Use

In certain aspects, the present disclosure provides methods of degrading an estrogen receptor in a subject, comprising administering to the subject a compound disclosed herein.

In certain aspects, the present disclosure provides uses of a compound disclosed herein in the manufacture of a medicament for degrading an estrogen receptor in a subject.

In certain aspects, the present disclosure provides compounds disclosed herein for use in degrading an estrogen receptor in a subject.

In certain aspects, the present disclosure provides methods of treating or preventing a disease or disorder in a subject in need thereof, comprising administering to the subject a compound disclosed herein (e.g., in a therapeutically effective amount).

In certain aspects, the present disclosure provides methods of treating a disease or disorder in a subject in need thereof, comprising administering to the subject a compound disclosed herein (e.g., in a therapeutically effective amount).

In certain aspects, the present disclosure provides uses of a compound disclosed herein in the manufacture of a medicament for treating or preventing a disease or disorder in a subject in need thereof.

In certain aspects, the present disclosure provides uses of a compound disclosed herein in the manufacture of a medicament for treating a disease or disorder in a subject in need thereof.

In certain aspects, the present disclosure provides compounds disclosed herein for use in treating or preventing a disease or disorder in a subject in need thereof.

In certain aspects, the present disclosure provides compounds disclosed herein for use in treating a disease or disorder in a subject in need thereof.

In certain embodiments, the disease or disorder is an estrogen receptor-mediated disease or disorder.

In certain embodiments, the disease or disorder is cancer.

In certain embodiments, the disease or disorder is breast cancer, lung cancer, ovarian cancer, endometrial cancer, prostate cancer, or esophageal cancer.

In certain embodiments, the cancer includes, but are not limited to, one or more of the cancers of Table A.

TABLE A

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal carcinoma | endocrine gland | endodermal sinus | enteropathy- |
| | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton | mantle cell |
| histiocytoma | nerve sheath tumor | tumor | lymphoma |
| marginal zone B-cell | mast cell leukemia | mediastinal germ cell | medullary carcinoma |
| lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal zone | squamous cell | synovial sarcoma | Sezary's disease |
| lymphoma | carcinoma | | |
| small intestine cancer | squamous carcinoma | stomach cancer | T-cell lymphoma |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In certain embodiments, the cancer is a solid tumor. In certain embodiments, the cancer a hematological cancer. Exemplary hematological cancers include, but are not limited to, the cancers listed in Table B. In certain embodiments, the hematological cancer is acute lymphocytic leukemia, chronic lymphocytic leukemia (including B-cell chronic lymphocytic leukemia), or acute myeloid leukemia.

TABLE B

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's macroglobulinemia | T-cell lymphoma |
| (WM) | |
| diffuse large B-cell lymphoma | mast cell leukemia |
| (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell |
| | lymphoma |
| Richter's transformation | |

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric synthescs. See, for example, Jaeques et al., Enantiomers, Raccmates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p, 268 (E.F. Eliel, Ed., Univ, of Notre Dame Press, Notre Dame, IN 1972).

The disclosure additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆. C₁₋₅, C₁₄, C₁₋₃, C₁₋₂, C₂₋₆, C₂₋₅, C₂₋₄, C₂₋₃, C₃₋₆, C₃₋₅, C₃₄, C₄₆, C₄₋₅, and C₅₋₆ alkyl.

The following terms are intended to have the meanings presented therewith below and are useful in understanding the description and intended scope of the present disclosure. When describing the disclosure, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., -CH₃). In certain embodiments, the alkyl group is substituted C₁₋₁₀ alkyl. Common alkyl abbreviations include Me (-CH₃), Et (-CH₂CH₃), i-Pr (-CH(CH₃)₂), n-Pr (-CH₂CH₂CH₃), n-Bu (-CH₂CH₂CH₂CH₃), or i-Bu (-CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (-CH₂—), ethylene (-CH₂CH₂-), propylene (-CH₂CH₂CH₂-),butylene (-CH₂CH₂CH₂CH₂-), pentylene (-CH₂CH₂CH₂CH₂CH₂-), hexylene (-CH₂CH₂CH₂CH₂CH₂CH₂—), and the like. Exemplary substituted divalent alkylene groups, e.g., substituted with one or more alkyl (methyl) groups, include but are not limited to, substituted methylene (-CH(CH₃)—. (-C(CH₃)₂—), substituted ethylene (-CH(CH₃) CH₂-, -CH₂CH(CH₃)—, —C(CH₃); CH₂-, -CH₂C(CH₃)₂—), substituted propylene (-CH(CH₃) CH₂CH₂-, -CH₂CH(CH₃) CH₂-, -CH₂CH₂CH(CH₃)—, —C(CH₃)₂CH₂CH₂-, -CH₂C(CH₃)₂CH₂-, -CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkonyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (-CH═CH—) and propenylene (e.g., -CH═CHCH₂-, -CH₂—CH═CH—). Exemplary substituted divalent alkenylene groups, e.g., substituted with one or more alkyl (methyl) groups, include but are not limited to, substituted ethylene (-C(CH₃)═CH—, -CH═C(CH₃)-), substituted propylene (e.g., —C(CH₃)═CHCH₂-, -CH═C(CH₃) CH₂-, -CH═CHCH(CH₃)—, -CH═CHC(CH₃)₂-, -CH(CH₃)-CH═CH—, —C(CH₃)₂-CH═CH—, -CH₂—C(CH₃)═CH—, -CH₂—CH═C(CH₃)-), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl docs not contain any double bonds. In certain embodiments, an alkynyl group has 2 to 10 carbon atoms (“C_{2-1 0} alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently optionally substituted, i.e., unsubstituted (an “unsubstituted alkynyl”) or substituted (a “substituted alkynyl”) with one or more substituents: e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl.” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“C₁₋₁₀ heteroalkyl”). In certain embodiments, a heteroalkyl group is a saturated group having 1 to 9 carbon atoms and 1, 2, 3, or 4 heteroatoms (“C₁₋₉ heteroalkyl”). In certain embodiments, a heteroalkyl group is a saturated group having 1 to 8 carbon atoms and 1, 2, 3, or 4 heteroatoms (“C₁₋₈ heteroalkyl”). In certain embodiments, a heteroalkyl group is a saturated group having 1 to 7 carbon atoms and 1, 2, 3, or 4 heteroatoms (“C₁₋₇ heteroalkyl”). In certain embodiments, a heteroalkyl group is a group having 1 to 6 carbon atoms and 1, 2, or 3 heteroatoms (“C₁₋₆ heteroalkyl”). In certain embodiments, a heteroalkyl group is a saturated group having 1 to 5 carbon atoms and 1 or 2 heteroatoms (“C₁₋₅ heteroalkyl”). In certain embodiments, a heteroalkyl group is a saturated group having 1 to 4 carbon atoms and/or 2 heteroatoms (“C₁₄ heteroalkyl”). In certain embodiments, a heteroalkyl group is a saturated group having 1 to 3 carbon atoms and 1 heteroatom (“C₁₋₃ heteroalkyl”). In certain embodiments, a heteroalkyl group is a saturated group having 1 to 2 carbon atoms and 1 heteroatom (“C₁₋₂ heteroalkyl”). In certain embodiments, a heteroalkyl group is a saturated group having 1 carbon atom and 1 heteroatom (“C₁ heteroalkyl”). In certain embodiments, a heteroalkyl group is a saturated group having 2 to 6 carbon atoms and 1 or 2 heteroatoms (“C₂₋₆ heteroalkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted C₁₋₁₀ heteroalkyl. In certain embodiments, the heteroalkyl group is a substituted C₁₋₁₀ heteroalkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“C₂₋₁₀ heteroalkenyl”). In certain embodiments, a heteroalkenyl group has 2 to 9 carbon atoms at least one double bond, and 1, 2, 3, or 4 heteroatoms (“C₂₋₉ heteroalkenyl”). In certain embodiments, a heteroalkenyl group has 2 to 8 carbon atoms, at least one double bond, and 1, 2, 3, or 4 heteroatoms (“C₂₋₈ heteroalkenyl”). In certain embodiments, a heteroalkenyl group has 2 to 7 carbon atoms, at least one double bond, and 1, 2, 3, or 4 heteroatoms (“C₂₋₇ heteroalkenyl”). In certain embodiments, a heteroalkanyl group has 2 to 6 carbon atoms, at least one double bond, and 1, 2, or 3 heteroatoms (“C₂₋₆ heteroalkenyl”). In certain embodiments, a heteroalkenyl group has 2 to 5 carbon atoms, at least one double bond, and 1 or 2 heteroatoms (“C₂₋₅ heteroalkenyl”). In certain embodiments, a heteroalkenyl group has 2 to 4 carbon atoms, at least one double bond, and I or 2 heteroatoms (“C₂₋₄ heteroalkenyl”). In certain embodiments, a heteroalkenyl group has 2 to 3 carbon atoms, at least one double bond, and 1 heteroatom (“C₂₋₃ heteroalkenyl”). In certain embodiments, a heteroalkenyl group has 2 to 6 carbon atoms, at least one double bond, and 1 or 2 heteroatoms (“C₂₋₆ heteroalkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted C₂₋₁₀ heteroalkenyl. In certain embodiments, the heteroalkenyl group is a substituted C₂₋₁₀ heteroalkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“C₂₋₁₀ heteroalkynyl”). In certain embodiments, a heteroalkynyl group has 2 to 9 carbon atoms, at least one triple bond, and 1, 2, 3, or 4 heteroatoms (“C₂₋₉ heteroalkynyl”). In certain embodiments, a heteroalkynyl group has 2 to 8 carbon atoms, at least one triple bond, and 1, 2, 3, or 4 heteroatoms (“C₂₋₈ heteroalkynyl”). In certain embodiments, a heteroalkynyl group has 2 to 7 carbon atoms, at least one triple bond, and 1, 2, 3, or 4 heteroatoms (“C₂₋₇ heteroalkynyl”). In certain embodiments, a heteroalkynyl group has 2 to 6 carbon atoms, at least one triple bond, and 1, 2, or 3 heteroatoms (“C₂₋₆ heteroalkynyl”). In certain embodiments, a heteroalkynyl group has 2 to 5 carbon atoms, at least one triple bond, and 1 or 2 heteroatoms (“C₂₋₅ heteroalkynyl”). In certain embodiments, a heteroalkynyl group has 2 to 4 carbon atoms, at least one triple bond, and lor 2 heteroatoms (“C₂₋₄ heteroalkynyl”). In certain embodiments, a heteroalkynyl group has 2 to 3 carbon atoms, at least one triple bond, and 1 heteroatom (“C₂₋₃ heteroalkynyl”). In certain embodiments, a heteroalkynyl group has 2 to 6 carbon atoms, at least one triple bond, and 1 or 2 heteroatoms (“C₂₋₆ heteroalkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted C₂ 10 heteroalkynyl. In certain embodiments, the heteroalkynyl group is a substituted C₂₋₁₀ heteroalkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene.” “heteroalkenylene,” or “heteroalkynylene.” group, it is understood that the range or number refers to the range or number of carbons in the linear divalent chain. “Heteroalkylene.” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 x electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5- to 14-membered monocyclic or polycyclic 4n+2 aromatic ring system (e.g., having 6, 10, or 14×electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

5-membered heteroaryl containing one heteroatom includes, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Carbocyclyl” refers to a radical of a non-aromatic cyclic hydrocarbon group having from 3 to 12 ring carbon atoms (“C_{3-1 2} carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexanyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

In certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share one common bond with, one or more carbocyclyl groups, as defined above, wherein the point of attachment is on any of the fused rings. In such instances, the number of carbons designates the total number of carbons in the fused carbocyclyl ring system. When substitution is indicated, unless otherwise specified, substitution can occur on any of the fused rings.

“Spiro carbocyclyl” or or “spiro carbocycle” refers to ring systems wherein the carbocyclyl group, as defined above, form spiro structure with, i.e., share one common atom with, one or more carbocyclyl groups, as defined above, wherein the point of attachment is on any of the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on any of the carbocyclyl rings in which the spiro structure is embedded.

“Bridged carbocyclyl” or or “bridged carbocycle” refers to ring systems wherein the carbocyclyl group, as defined above, form bridged structure with, i.e., share more than one atoms (as such, share more than one bonds) with, one or more carbocyclyl groups, as defined above,
wherein the point of attachment is on any of the carbocyclyl rings in which the bridged structure is embedded. In such instances, the number of carbons designates the total number of carbons of the bridged rings. When substitution is indicated, unless otherwise specified, substitution can occur on any of the carbocyclyl rings in which the bridged structure is embedded.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl. tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

In certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share one common bond with, one or more heterocyclyl or carbocyclyl groups, as defined above, wherein the point of attachment is on any of the fused rings. In such instances, the number of carbons designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than one atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the bridged rings.

“Hetero” when used to describe a compound or a group present on a compound means that one or more carbon atoms in the compound or group have been replaced by a nitrogen, oxygen, sulfur, boron, phosphorus, or silicon heteroatom, as valency permits. Hetero may be applied to any of the hydrocarbyl groups described above having from 1 to 5, and particularly from 1 to 3 heteroatoms.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR^{Z}, wherein each R is independently alkyl, as defined herein, C₁₋₆ alkylamino refers to the group —NHR or —NR^{Z}, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (C₁), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl. amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), B-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri- iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn). S-t-butyl ester, silyl esters, and orthoesters) and oxazoline.

These and other exemplary substituents are described in more detail in the Detailed Description, Examples, and claims. The disclosure is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age. weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of aequiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in certain embodiments, to ameliorating the disease or disorder (i.e., arresting the disease or reducing the manifestation, extent or severity of at least one of the clinical symptoms thereof). In certain embodiments, “treating” or “treatment” refers to ameliorating at least one physical parameter, which may not be discernible by the subject. In certain embodiments, “treating” or “treatment” refers to modulating the disease or disorder, either physically. (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range. In certain embodiments, the number or numerical range vary by 1%, 2%, 3%, 4%, or 5% of the stated number or numerical range. In certain embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in certain embodiments, to A only (optionally including elements other than B); in certain embodiments, to B only (optionally including elements other than A); in certain embodiments, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of.” “only one of.” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) may refer, in certain embodiments, to at least one, optionally including more than one, A, with no B present (and optionally including elements other than B); in certain embodiments, to at least one, optionally including more than one. B, with no A present (and optionally including elements other than A); in certain embodiments, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### Examples

In order that the invention described herein may be more fully understood, the following examples are set forth. The examples described in this application are offered to illustrate the compounds, pharmaceutical compositions, and methods provided herein and are not to be construed in any way as limiting their scope.

It is understood that the values presented in the examples are approximate values, and they are subject to instrumental and/or experimental variations.

The following abbreviations were used in descriptions and examples:
ACN acetonitrile; AIBN azobisisobutyronitrile; BINAP ([1,1′-Binaphthalene]-2.2′-diyl)bis(diphenylphosphane); BPO dibenzoyl peroxide; DCE 1,2-dichloroethane; DCM dichloromethane; DEAD Diethylazodicarboxylate; DIPEA N,N-diisopropylethylamine; DMF N,N-dimethylformamide; DMA N,N-dimethylacetamide; DMSO dimethylsulfoxide; EA ethyl acetate; FA formic acid; HMTA 1,3,5,7-Tetraazaadamantane; hr Hour; hrs Hours; IPA iso-propyl alcohol; IPE di- isopropyl ether; K₂CO₃ Potassium carbonate; m-CPBA 3-chlorobenzenecarboperoxoic acid; LC/MS liquid chromatography-mass spectrometry; MeOH methanol; MS mass spectrometry; mL Milliliters; NaBH₃CN Sodium cyanoborohydride; NBS N-bromosuccinimide; NCS N-chlorosuccinimide; NMP N-methyl pyrrolidinone; NMR nuclear magnetic resonance; PE petroleum ether; ppm parts per million; T3P propanephosphonic acid anhydride; TEA triethylamine; THF tetrahydrofuran

### I. Synthesis and Characterization of Intermediates and “A” Compounds

The chemical reagents were purchased from commercial sources (such as Alfa, Acros, Sigma Aldrich, TCI, and Shanghai Chemical Reagent Company), and used without further purification.

In obtaining the compounds described in the examples below and the corresponding analytical data, the following experimental and analytical protocols were followed unless otherwise indicated.

A summary of LC-MS methods is shown below.

### Method A:

Waters SunFire C18 50*4.6 mm 5 nm 2.000 ml/min 2.6 min Column Temperature: 40° C.
Gradient: 5% B hold for 0.2 min, increase to 95% B within 1,40 min, hold at 95% B for 0.9 min, then back to 5% B within 0.01 min
Pump A: 0.1% formic acid (FA) and 10% acetonitrile (ACN) in H₂O
Pump B: 0.1% FA and 10% H₂O in ACN.

### Method B:

Waters SunFire C18 50*4.6 mm 5 um 2.000 ml/min 2.6 min Column Temperature: 40° C.
Gradient: 5% B hold for 0.2 min, increase to 95% B within 1,40 min, hold at 95% B for0.9 min. then back to 5% B within 0.01 min
Pump A: 0.03% trifluoroacetic acid (TFA) in H₂O
Pump B: 0.03% TFA in ACN

### Method C:

Column: Sunfire C18 150*4.6 mm 5 um 1.00 ml/min Column Temperature: 40° C.
Gradient: 10% B hold for 1.8 min, increase to 95% B within 10.2 min, hold at 95% B for 3.0 min, then back to 10% B within 0.01 min
Pump A: 0.03% TFA in H₂O
Pump B: 0.03% TFA in ACN

### Method D:

Column: Luna C18 30*2.0 mm 3 um 1,200 ml/min 1.5 min Column Temp.: 50° C. 5% B increase to 95% B within 0.7 min, hold at 95% B for 0.4 min, back to 5% B within 0.01 min
Pump A: 0.03% TFA in H₂O
Pump B: 0.03% TFA in ACN

### Method E:

SunFire C18 50*4.6 mm 5 um 2.6 min 2.0 ml/min
Temperature: 40° C.
Gradient: 10% B increase to 30% B for 0.40 min, increase to 95% B within 1.60 min, 95% B hold for 0.90 min, back to 10% B within 0.01 min, A⁷⁰B30

### Method F:

SunFire C18 50*4.6 mm 5 um 2.6 min 2.0 ml/min
Temperature: 40° C.
Gradient: 10% B increase to 30% B for 0.40 min, increase to 95% B within 1.60 min, 95% B hold for 0.90 min, back to 10% B within 0.01 min, A⁵⁰B50.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Compound purification was carried out as needed using a variety of traditional methods including, but not limited to, preparative chromatography under acidic, neutral, or basic conditions using either normal phase or reverse phase HPLC or flash columns or Prep-TLC plates.

Flash chromatography was performed on a Biotage Isolera One via column with silica gel particles of 200-300 mesh. Analytical and preparative thin-layer chromatography was performed using silica gel 60 GF254 plates. Normal-phase silica gel chromatography (FCC) was also performed on silica gel (SiO₂) using prepacked cartridges.

Preparative reverse-phase high performance liquid chromatography (RP HPLC) was performed on either:
METHOD 1. Prep-HPLC with Waters-Sunfire C18 21,2×250 mm×10 um, and mobile phase of 10-20% ACN in water (0.1% HCOOH) over 15 min and then hold at 100% ACN for 5 min, at a flow rate of 20 mL/min, or
METHOD 2.

Preparative supercritical fluid high performance liquid chromatography (SFC) was performed either on a Waters 150 Prep-SFC system from Waters. The ABPR was set to 100 bar to keep the CO₂ in SF conditions, and the flow rate may verify according to the compound characteristics, with a flow rate ranging from 70 g/min to 140 g/min. The column temperature was ambient temperature

Nuclear magnetic resonance (NMR) spectra were recorded using Brucker AVANCE NEO 400 MHz at around 20-30° C. unless otherwise specified. The following abbreviations are used: s, singlet; d, doublet; t, triplet; q, quartet; m, multiplet; dd, doublet of doublets; ddd, doublet of doublet of doublet; dt, doublet of triplets; bs, broad signal. Chemical shifts were reported in parts per million (ppm, 8) downfield from tetramethylsilane. It will be understood that for compounds comprising an exchangeable proton, said proton may or may not be visible on an NMR spectrum depending on the choice of solvent used for running the NMR spectrum and the concentration of the compound in the solution.

Mass spectra (MS) were obtained on a SHIMADZU LC-MS-2020 MSD using electrospray ionization (ESI) in positive mode unless otherwise indicated. Calculated (calcd.) mass corresponds to the exact mass.

Chemical names were generated using ChemDraw Ultra 12.0, ChemDraw Ultra 14.0 (CambridgeSoft Corp., Cambridge, MA) or ACD/Name Version 10.01 (Advanced Chemistry).

Compounds designated as R* or S* are enantiopure compounds where the absolute configuration was not determined.

### Intermediate 1:1-(4-(6-(benzyloxy)-2-phenyl-3,4-dihydronaphthalen-1-yl)-3-methoxyphenyl)-4-(dimethoxymethyl)piperidine

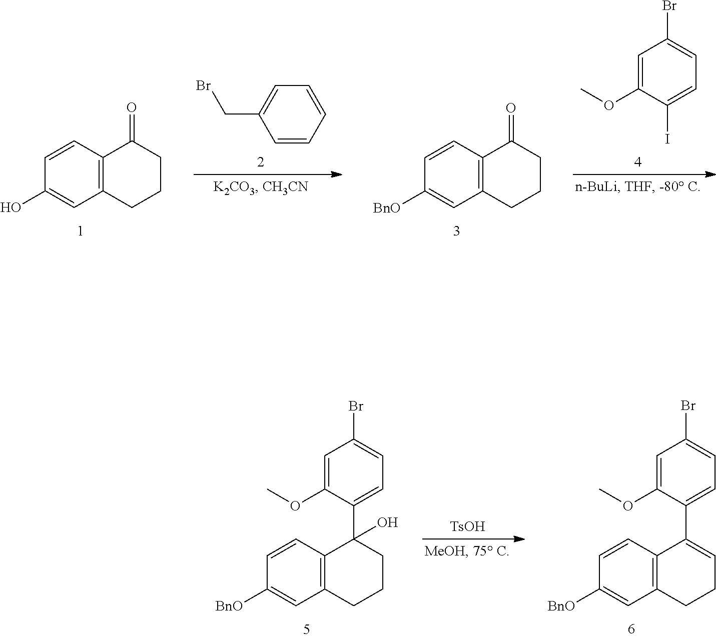

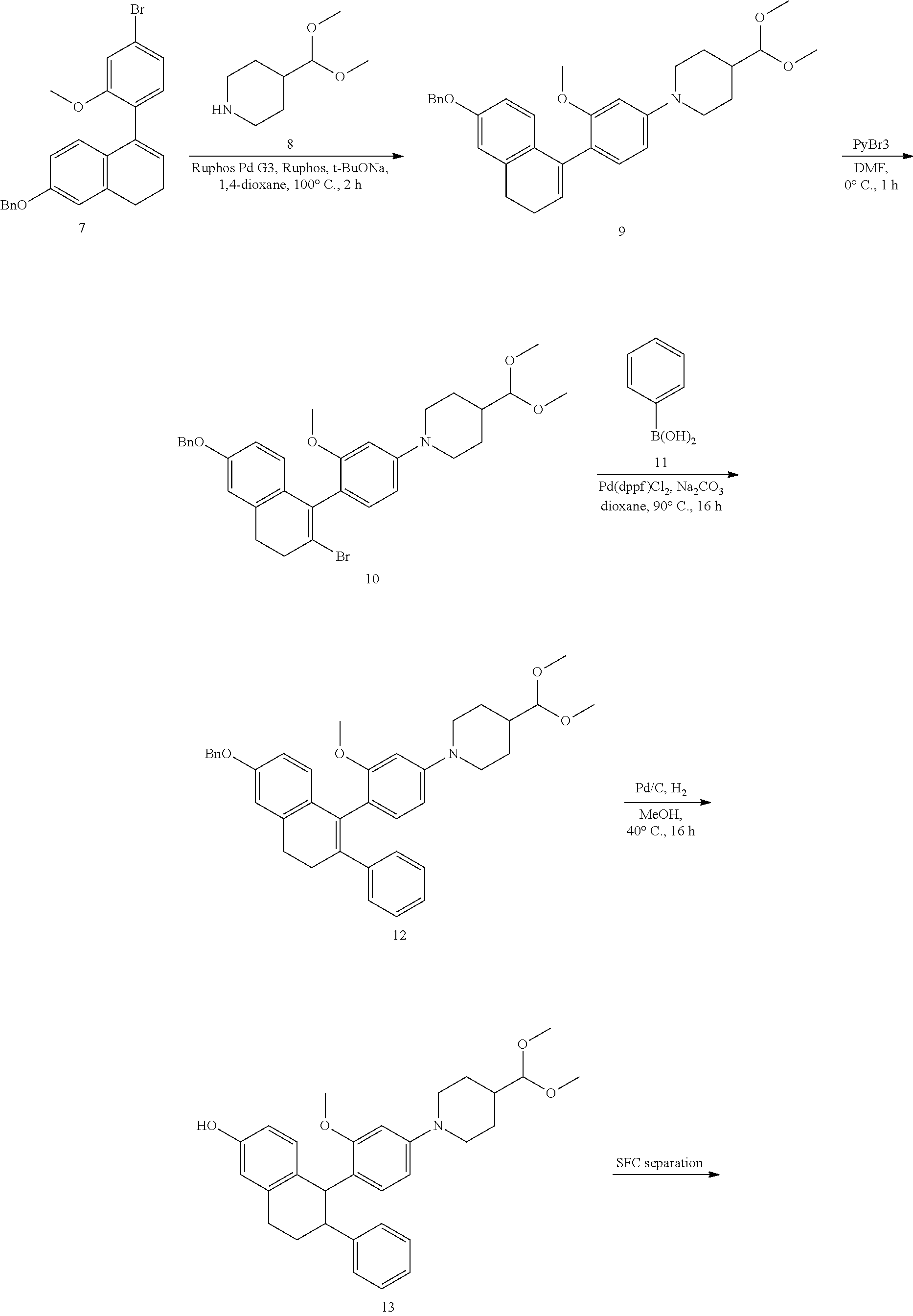

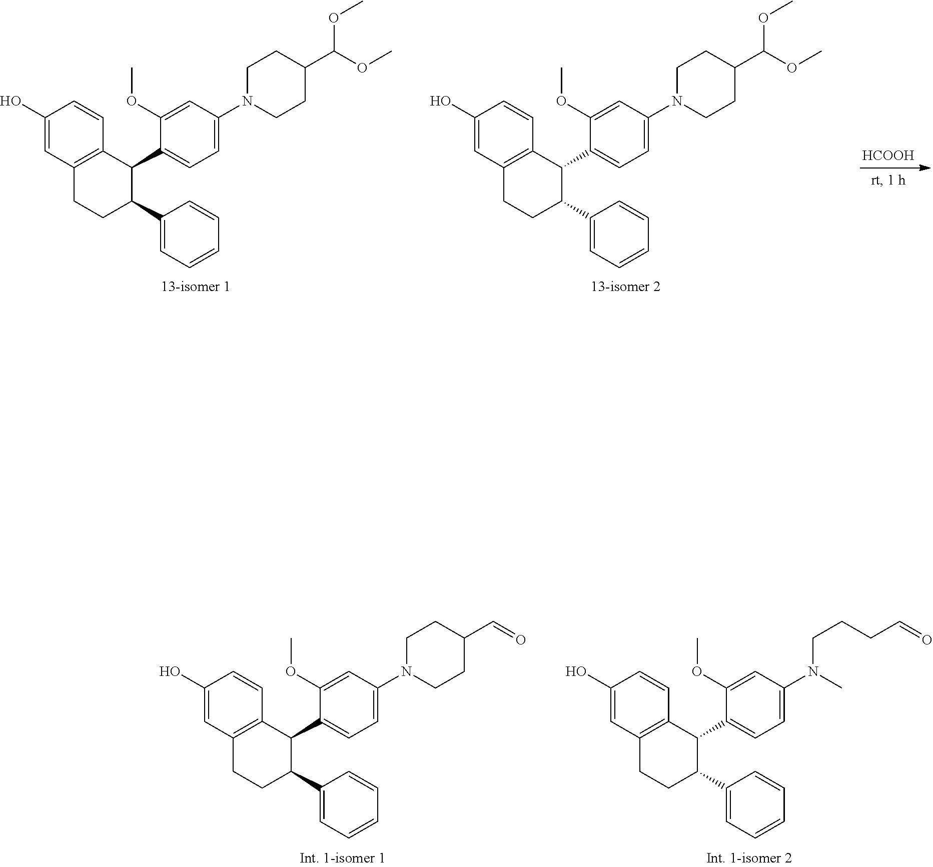 [SMILES: COc1cc(N2CCC(C(OC)OC)CC2)ccc1[C@@H]1c2ccc(O)cc2CC[C@@H]1c1ccccc1 ; COc1cc(N2CCC(C(OC)OC)CC2)ccc1[C@H]1c2ccc(O)cc2CC[C@H]1c1ccccc1 ; "HCOOH" ; COc1cc(N2CCC(C=O)CC2)ccc1[C@@H]1c2ccc(O)cc2CC[C@@H]1c1ccccc1 ; COc1cc(N(C)CCCC=O)ccc1[C@H]1c2ccc(O)cc2CC[C@H]1c1ccccc1]

### Step 1:6-(benzyloxy)-3,4-dihydronaphthalen-1 (2H)-one

To a solution of 6-hydroxy-3,4-dihydronaphthalen-1 (2H)-one (25 g, 154.14 mmol, 1 eq) and K₂CO₃ (46.2 g, 308.28 mmol, 2 eq) in CH₃CN(350 mL) was added BnBr (3.1 g, 184.96 mmol, 1,2 eq) and stirred at 50° C. for 2 hours. LCMS showed the reaction was completed. The reaction was concentrated under vacuum to afford the product 6-(benzyloxy)-3,4-dihydronaphthalen-1 (2H)-one (30.3 g,78%). LC-MS purity 100% (UV at 254 nm), 253 [M+H]⁺.

### Step 2:6-(benzyloxy)-1-(4-bromo-2-methoxyphenyl)-1,2,3,4-tetrahydronaphthalen-1-ol

To a solution of 4-bromo-1-iodo-2-methoxybenzene (14.9 g, 47.6 mmol, 1,2 eq.) in THF (100 mL) cooled to −80° C. was added at n-BuLi (2.5 M, 19.1 mL, 47.6 mmol, 1,2 eq.) and stirred at for 1 hour under N₂. Then 6-(benzyloxy)-3,4-dihydronaphthalen-1 (2H)-one (10 g, 39.6 mmol, 1,2 eq.) in THF (30 mL) was added and stirred at −80° C. for 3 hours. Once the reaction was completed, the mixture was quenched with H₂O (200 mL) and extracted with EA (400 mL) to give crude product. The residue was purified by column chromatography on silica gel (PE:EA=10:1) to afford the product 6-(benzyloxy)-1-(4-bromo-2-methoxyphenyl)-1,2,3,4-tetrahydronaphthalen-1-ol (7.3 g, 42%) as an oil.

LC-MS purity: 100% (UV at 254 nm), 439, 451 [M+H]⁺.

### Step 3:7-(benzyloxy)-4-(4-bromo-2-methoxyphenyl)-1,2-dihydronaphthalene

To a mixture of 6-(benzyloxy)-1-(4-bromo-2-methoxyphenyl)-1,2,3,4-tetrahydronaphthalen-1-ol (7 g, 15.98 mmol, 1 eq) in MeOH (70 mL) was added TsOH (60 mg, 0.32 mmol, 0.02 eq) and stirred at 50° C. for 0.5 hour. LCMS showed the reaction was completed. The mixture was concentrated to afford 7-(benzyloxy)-4-(4-bromo-2-methoxyphenyl)-1,2-dihydronaphthalene (5.8 g, 86%) as white solid. LC-MS purity: 99.26% (UV at 254 nm), 421, 423 [M+H]⁺.

### Step 4:1-(4-(6-(benzyloxy)-3,4-dihydronaphthalen-1-yl)-3-methoxyphenyl)-4-(dimethoxymethyl)piperidine

Ruphos (134.0 mg, 0.286 mmol, 0.2 eq), Ruphos Pd G3 (240.0 mg, 0.286 mmol, 0.2 eq) and t-BuONa (550.0 mg, 5.72 mmol, 4.0 eq) was added to a degassed solution of 7-(benzyloxy)-4-(4-bromo-2-methoxyphenyl)-1,2-dihydronaphthalene (600.0 mg, 1,428 mmol, 1.0 eq) and 4-(dimethoxymethyl)piperidine (340.2 mg, 2.14 mmol, 1.5 eq) in 1,4-dioxane (30 mL). The reaction mixture was heated to 100° C. for 2 hrs. LC-MS showed the reaction was completed. The reaction mixture was cooled to rt, filtered, the precipitate was washed with THF (30 mL×2), the filtrate was evaporated, the residue was purified by SiO₂ column chromatography (EtOAc:PE=1:5) to afford 1-(4-(6-(benzyloxy)-3,4-dihydronaphthalen-1-yl)-3-methoxyphenyl)-4-(dimethoxymethyl)piperidine (530 mg, 74.3%) as a yellow oil. LC-MS purity: 98.3% (UV at 254 nm), 500.1 [M+H]⁺. ¹H NMR (400 MHz, CDCl₃) δ 7.43-7.32 (m, 5H), 7.03 (d, J=8.0 Hz, 1H), 6.80 (d, J=2.4 Hz, 1H), 6.69-6.62 (m, 2H), 6.55-6.53 (m, 2H), 5.86 (t, J=4.4 Hz, 1H), 5.03 (s, 2H), 4.10 (d, J=7.6 Hz, 1H), 3.75-3.72 (m, 2H), 3.68 (s, 3H), 3.39 (s, 6H), 2.87-2.83 (m, 2H), 2.75-2.68 (m, 2H), 2.42-2.36 (m, 2H), 1.89-1.86 (m, 2H), 1.80-1.75 (m, 1H), 1.53-1.43 (m, 2H),

### Step 5:11-(4-(6-(benzyloxy)-2-bromo-3,4-dihydronaphthalen-1-yl)-3-methoxyphenyl)-4-(dimethoxymethyl)piperidine

To a mixture of 1-(4-(6-(benzyloxy)-3,4-dihydronaphthalen-1-yl)-3-methoxyphenyl)-4-(dimethoxymethyl)piperidine (1.85 g, 3.7 mmol, 1 eq) in DMF (20 mL) was added PyBr₃ (1,18 g. 3.7 mmol, 1 eq) in DMF (10 mL) dropwise slowly at 0° C. over 30 minutes, then stirred at 0° C. for 1 hour. LC-MS showed the reaction was completed, 10 mL of Sat. NH₄Cl solution was added, followed by 110 mL of water, extracted with EtOAc (120 mL×3). The combined organic layers were washed with water (120 mL×2), brine (120 mL), dried over Na₂SO₄, filtered, the filtrate was evaporated. The residue was purified by Chem-flash to afford 1-(4-(6-(benzyloxy)-2-bromo-3,4-dihydronaphthalen-1-yl)-3-methoxyphenyl)-4-(dimethoxymethyl)piperidine (1.51 g, 70.1%) as a yellow solid.

LC-MS purity: 88.4% (UV at 254 nm), 580.2 [M+H]⁺.

### Step 6:1-(4-(6-(benzyloxy)-2-phenyl-3,4-dihydronaphthalen-1-yl)-3-methoxyphenyl)-4-(dimethoxymethyl)piperidine

Pd(dppf)Cl₂ (227 mg, 0.311 mmol, 0.1 eq), and Na₂CO₃ (660 mg, 6.22 mmol, 2.0 eq) was added to a degassed solution of 1-(4-(6-(benzyloxy)-2-bromo-3,4-dihydronaphthalen-1-yl)-3-methoxyphenyl)-4-(dimethoxymethyl)piperidine (1.79 g, 3.11 mmol, 1.0 eq) and phenylboronic acid (532 mg, 4.35 mmol, 1,4 eq) in 1,4-dioxane/H₂O (40 mL/4 mL). The reaction mixture was heated to 90° C. for 16 hours. LC-MS showed the reaction was completed. The reaction mixture was cooled to room temperature, filtered, the precipitate was washed with THF (40 mL×2), the filtrate was evaporated, the residue was purified by Chemflash to afford 1-(4-(6-(benzyloxy)-2-phenyl-3,4-dihydronaphthalen-1-yl)-3-methoxyphenyl)-4-(dimethoxymethyl)piperidine (1,48 g, 82.8%) as a yellow oil.

LC-MS purity: 99.4% (UV at 254 nm), 576.1 [M+H]⁺. ¹H NMR (400 MHz, CDCl₃) δ 7.43-7.32 (m, 5H), 7.10-6.99 (m, 5H), 6.83 (d, J=2.4 Hz, 1H), 6.74 (d, J=8.0 Hz, 1H), 6.68-6.61 (m, 2H), 6.40-6.37 (m, 2H), 5.05 (s, 2H), 4.08 (d, J=7.6 Hz, 1H), 3.68-3.64 (m, 2H), 3.51 (s, 3H), 3.37 (s, 6H), 2.98-2.92 (m, 2H), 2.80-2,76 (m, 2H), 2.68-2.62 (m, 2H), 1.87-1.84 (m, 2H), 1.77-1.73 (m, 1H), 1.51-1.44 (m, 2H),

### Step 7: 5-(4-(4-(dimethoxymethyl)piperidin-1-yl)-2-methoxyphenyl)-6-phenyl-5,6,7,8-tetrahydronaphthalen-2-ol

A mixture of 1-(4-(6-(benzyloxy)-2-phenyl-3,4-dihydronaphthalen-1-yl)-3-methoxyphenyl)-4-(dimethoxymethyl)piperidine (1,48 g, 2.57 mmol, 1.0 eq) and Pd/C(700 mg) in MeOH (150 mL) was degassed under reduced pressure, purged with H₂ atmosphere, The reaction mixture was heated to 40° C. for 16 hours. LC-MS showed the reaction was completed. The reaction mixture was cooled to room temperature, filtered, the precipitate was washed with EtOAc (20 mL×2), the filtrate was evaporated, the residue was purified by Chemflash to afford 5-(4-(4-(dimethoxymethyl)piperidin-1-yl)-2-methoxyphenyl)-6-phenyl-5,6,7,8-tetrahydronaphthalen-2-ol (1,12 g, 89.4%) as a yellow solid.

LC-MS purity: 99.4% (UV at 254 nm), 488.3 [M+H]⁺. ¹H NMR (400 MHz, CDCl₃) δ 7.10-7.03 (m, 3H), 6.79-6.76 (m, 3H), 6.65 (s, 1H), 6.51-6.48 (m, 2H), 6.34 (d, J=8.0 Hz, 1H), 6.10 (s, 1H), 4.77 (d, J=4.8 Hz, 1H), 4.07 (d, J=7.2 Hz, 1H), 3.60-3.57 (m, 2H), 3.36 (s, 6H), 3.29-3.25 (m, 1H), 3.00-2.97 (m, 5H), 2.59 (t, J=11,2 Hz, 2H), 2.32-2.21 (m, 1H), 1.83-1.80 (m, 2H), 1.73-1.68 (m, 3H), 1.49-1.43 (m, 2H),

### Step 8: (5S,6S)-5-(4-(4-(dimethoxymethyl)piperidin-1-yl)-2-methoxyphenyl)-6-phenyl-5,6,7,8-tetrahydronaphthalen-2-ol & (5R,6R)-5-(4-(4-(dimethoxymethyl)piperidin-1-yl)-2-methoxyphenyl)-6-phenyl-5,6,7,8-tetrahydronaphthalen-2-ol

5-(4-(4-(dimethoxymethyl)piperidin-1-yl)-2-methoxyphenyl)-6-phenyl-5,6,7,8-tetrahydronaphthalen-2-ol (1,12 g, 2.3 mmol) was separated by SFC to afford (5S,6S)-5-(4-(4-(dimethoxymethyl)piperidin-1-yl)-2-methoxyphenyl)-6-phenyl-5,6,7,8-tetrahydronaphthalen-2-ol (550 mg) & (5R,6R)-5-(4-(4-(dimethoxymethyl)piperidin-1-yl)-2-methoxyphenyl)-6-phenyl-5,6,7,8-tetrahydronaphthalen-2-ol (550 mg).

### Step 9:1-(4-((1S,2S)-6-hydroxy-2-phenyl-1,2,3,4-tetrahydronaphthalen-1-yl)-3-methoxyphenyl)piperidine-4-carbaldehyde & 1-(4-((1R,2R)-6-hydroxy-2-phenyl-1,2,3,4-tetrahydronaphthalen-1-yl)-3-methoxyphenyl)piperidine-4-carbaldehyde

A mixture of (5S,6S)-5-(4-(4-(dimethoxymethyl)piperidin-1-yl)-2-methoxyphenyl)-6-phenyl-5,6,7,8-tetrahydronaphthalen-2-ol (100 mg) or (5R,6R)-5-(4-(4-(dimethoxymethyl)piperidin-1-yl)-2-methoxyphenyl)-6-phenyl-5,6,7,8-tetrahydronaphthalen-2-ol (100 mg) and formic acid (5 mL) was stirred at room temperature for 1 h. The mixture was concentrated under reduced pressure to afford 1-(4-((1S,2S)-6-hydroxy-2-phenyl-1,2,3,4-tetrahydronaphthalen-1-yl)-3-methoxyphenyl)piperidine-4-carbaldehyde & 1-(4-((1R,2R)-6-hydroxy-2-phenyl-1,2,3,4-tetrahydronaphthalen-1-yl)-3-methoxyphenyl)piperidine-4-carbaldehyde (105 mg, crude) as dark red oil, which was used directly without further purification.

LC-MS purity: 99.5% (UV at 254 nm), 442.2 [M+H]⁺.

### Intermediate 2:5-(4-(3-(dimethoxymethyl)-1-oxa-8-azaspiro[4.5]decan-8-yl)-2-methoxyphenyl)-6-phenyl-5,6,7,8-tetrahydronaphthalen-2-ol

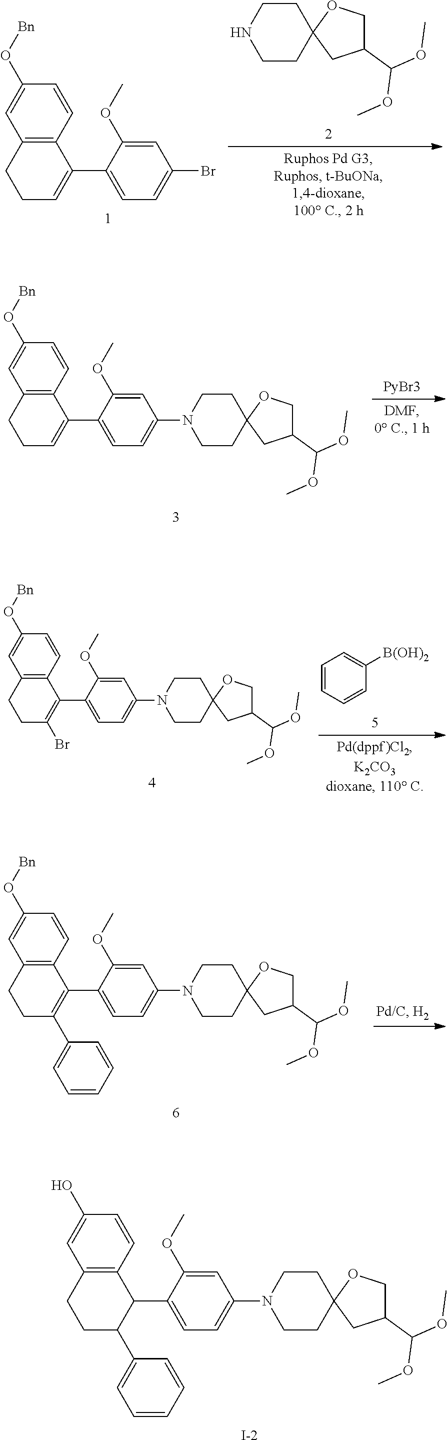

### Step 2-1

### 8-(4-(6-(benzyloxy)-3,4-dihydronaphthalen-1-yl)-3-methoxyphenyl)-3-(dimethoxymethyl)-1-oxa-8-azaspiro[4.5]decane

To a solution of 7-(benzyloxy)-4-(4-bromo-2-methoxyphenyl)-1,2-dihydronaphthalene (1,1 g, 2.61 mmol, 1 eq), 3-(dimethoxymethyl)-1-oxa-8-azaspiro[4.5]decane (562.1 mg, 2.61 mmol, 1 eq), t-BuONa (752.7 mg, 7.83 mmol, 3.0 eq) and Ruphos (121.6 mg, 0.26 mmol, 0.1 eq) in 1,4-dioxane (20 mL) was added Ruphos Pd G3 (218.2 mg, 0.26 mmol, 0.1 eq) under N₂ atmosphere. The mixture was heated to 100° C. for 2 hrs. LC-MS showed the reaction was completed. The reaction mixture was cooled to rt, filtered, the precipitate was washed with THF (30 mL×2), the filtrate was evaporated, the residue was purified by SiO₂ column chromatography (EtOAc:PE=1:5) to afford 8-(4-(6-(benzyloxy)-3,4-dihydronaphthalen-1-yl)-3-methoxyphenyl)-3-(dimethoxymethyl)-1-oxa-8-azaspiro[4.5]decane (1,2 g, 82.7%) as a yellow oil.

LC-MS purity: 100% (UV at 254 nm), LC-MS: 556.2 [M+H]⁺.

### Step 2-2

### 8-(4-(6-(benzyloxy)-2-bromo-3,4-dihydronaphthalen-1-yl)-3-methoxyphenyl)-3-(dimethoxymethyl)-1-oxa-8-azaspiro[4.5]decane

To a mixture of 8-(4-(6-(benzyloxy)-3,4-dihydronaphthalen-1-yl)-3-methoxyphenyl)-3-(dimethoxymethyl)-1-oxa-8-azaspiro[4.5]decane (1,2 g, 2.16 mmol, 1 eq) in DMF (20 mL) was added PyBr₃ (686.3 mg, 2.16 mmol, 1 eq) in DMF (10 mL) dropwise slowly at 0° C. over 30 minutes, then stirred at 0° C. for 1 hour. LC-MS showed the reaction was completed, 10 mL of Sat. NH₄Cl solution was added, followed by 110 mL of water, extracted with EtOAc (120 mL×3). The combined organic layers was washed with water (120 mL×2), brine (120 mL), dried over Na₂SO₄, filtered, the filtrate was evaporated. The residue was purified by Chem-flash to afford 8-(4-(6-(benzyloxy)-2-bromo-3,4-dihydronaphthalen-1-yl)-3-methoxyphenyl)-3-(dimethoxymethyl)-1-oxa-8-azaspiro[4.5]decane (900 mg, 65.7%) as a yellow solid.

LC-MS purity: 100% (UV at 254 nm), LC-MS: 634.1, 636.3 [M+H]⁺.

### Step 2-3

### 8-(4-(6-(benzyloxy)-2-phenyl-3,4-dihydronaphthalen-1-yl)-3-methoxyphenyl)-3-(dimethoxymethyl)-1-oxa-8-azaspiro[4.5]decane

To a solution of compound 1 (900 mg, 1,42 mmol, 1 eq) in dioxane (20 mL) was added compound 2 (207.98 mg, 1.70 mmol, 1,2 eq), H₂O (2 mL), K₂CO₃ (386.73 mg, 2.84 mmol, 2 eq) and Pd(dppf)Cl₂ (51.9 mg, 0.07 mmol, 0.05 eq) under N₂ atmosphere. The mixture was heated to 100° C. for 2 hrs. The solution is red and turbid. LC-MS showed the starting material was consumed completely and desired compound was detected. The mixture was concentrated to give a residue. The residue was purified by Chemflash to give 8-(4-(6-(benzyloxy)-2-phenyl-3,4-dihydronaphthalen-1-yl)-3-methoxyphenyl)-3-(dimethoxymethyl)-1-oxa-8-azaspiro[4.5]decane (815 mg, yellow, oil, yield 90.96%). LC-MS purity: 100% (UV at 254 nm), LC-MS: 632.1 [M+H]⁺.

### Step 2-4

5-(4-(3-(dimethoxymethyl)-1-oxa-8-azaspiro[4.5]decan-8-yl)-2-methoxyphenyl)-6-phenyl-5,6,7,8-tetrahydronaphthalen-2-ol

To a solution of 8-(4-(6-(benzyloxy)-2-phenyl-3,4-dihydronaphthalen-1-yl)-3-methoxyphenyl)-3-(dimethoxymethyl)-1-oxa-8-azaspiro[4.5]decane (815 mg, 1,29 mmol, 1 eq) in MeOH (15 mL) was added Pd/C(82 mg, 0.38 mmol, 0.3 eq) under H₂ (15 Psi) atmosphere. The mixture was heated to 40° C. for 16 hrs. The solution is black and turbid. LC-MS showed the starting material was consumed completely and desired compound was detected. The mixture was filtered and concentrated to give a residue. The residue was purificd by Chemflash to give 5-(4-(3-(dimethoxymethyl)-1-oxa-8-azaspiro[4.5]decan-8-yl)-2-methoxyphenyl)-6-phenyl-5,6,7,8-tetrahydronaphthalen-2-ol (640 mg, yellow, oil, yield 91,25%).

LC-MS purity: 100% (UV at 254 nm), LC-MS: 544.2 [M+H]⁺.

### Intermediate 3:5-(4-(2-(dimethoxymethyl)-7-azaspiro[3.5]nonan-7-yl)-2-methoxyphenyl)-6-phenyl-5,6,7,8-tetrahydronaphthalen-2-ol

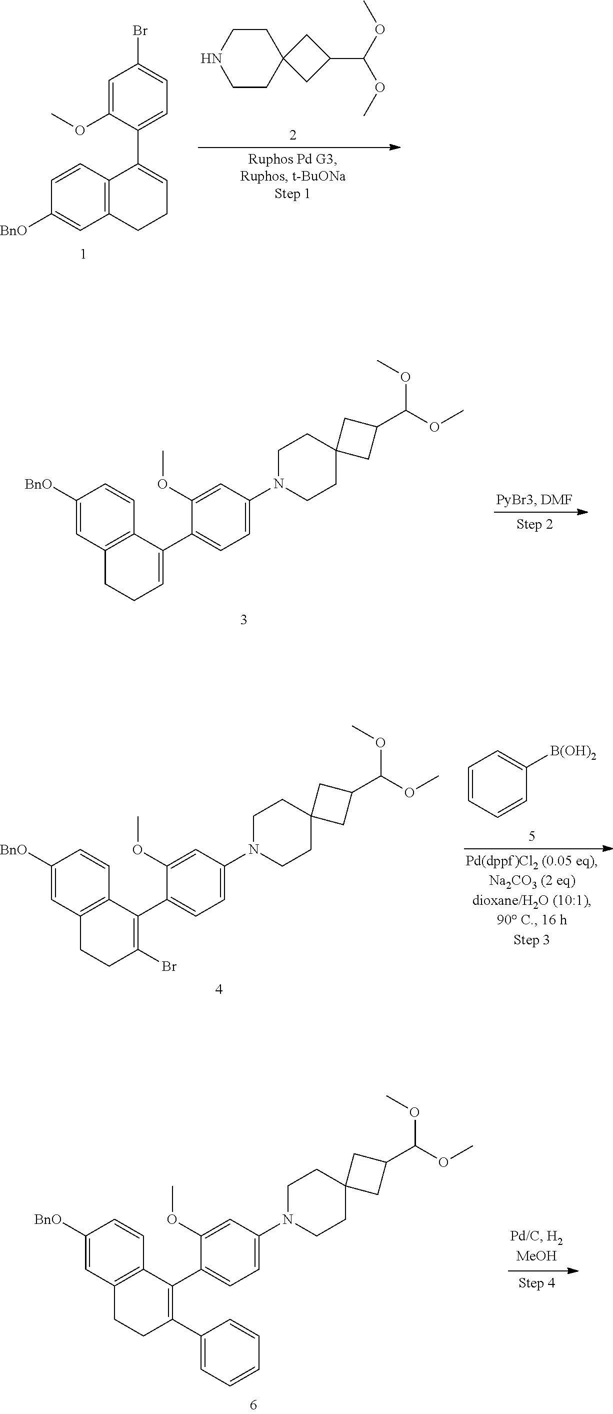

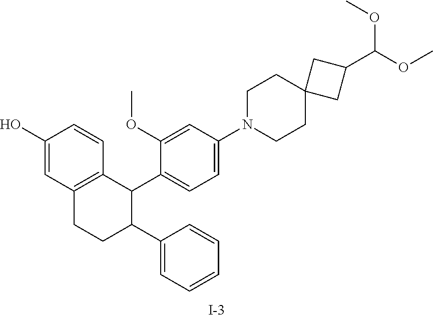

### Step 3-1:7

### (4-(6-(benzyloxy)-3,4-dihydronaphthalen-1-yl)-3-methoxyphenyl)-2-(dimethoxymethyl)-7-azaspiro[3.5]nonane

Ruphos (332.0 mg, 0.712 mmol, 0.2 eq), Ruphos Pd G3 (596.0 mg, 0.712 mmol, 0.2 eq) and t-BuONa (1.37 g, 14.2 mmol, 4.0 eq) was added to a degassed solution of 7-(benzyloxy)-4-(4-bromo-2-methoxyphenyl)-1,2-dihydronaphthalene (1.5 g, 3.56 mmol, 1.0 eq) and 2-(dimethoxymethyl)-7-azaspiro[3.5]nonane (710.0 mg, 3.56 mmol, 1.0 eq) in 1,4-dioxane (20 mL). The reaction mixture was heated to 100° C. and stirred for 16 hrs. LC-MS showed the reaction was completed. The reaction mixture was cooled to rt, the mixture was diluted with water and washed with EA, the organic phase was dried with Na₂SO₄ and concentrated under vacuum. The residue was purified by SiO₂ column chromatography (EtOAc:PE=1:5) to afford 7-(4-(6-(benzyloxy)-3,4-dihydronaphthalen-1-yl)-3-methoxyphenyl)-2-(dimethoxymethyl)-7-azaspiro[3.5]nonane (856 mg, 45%) as a yellow oil.

LC-MS purity: 100% (UV at 254 nm), MS: 540.3 [M+H]⁺.

### Step 3-2

7-(4-(6-(benzyloxy)-2-bromo-3,4-dihydronaphthalen-1-yl)-3-methoxyphenyl)-2-(dimethoxymethyl)-7-azaspiro[3.5]nonane

To a mixture of 7-(4-(6-(benzyloxy)-3,4-dihydronaphthalen-1-yl)-3-methoxyphenyl)-2-(dimethoxymethyl)-7-azaspiro[3.5]nonane (856 mg, 1.59 mmol, 1 eq) in DMF (20 mL) was added PyBr₃ (507.8 mg, 1.59 mmol, 1 eq) in DMF (10 mL) dropwise slowly at 0° C. over 30 minutes, then stirred at 0° C. for 1 hour. LC-MS showed the reaction was completed, 10 mL of Sat. NH₄Cl solution was added, followed by water, extracted with EtOAc. The combined organic layers was washed with water, brine, dried over Na₂SO₄, filtered, the filtrate was evaporated. The residue was purified by Chem-flash to afford 7-(4-(6-(benzyloxy)-2-bromo-3,4-dihydronaphthalen-1-yl)-3-methoxyphenyl)-2-(dimethoxymethyl)-7-azaspiro[3.5]nonane (750 mg, 76%) as a yellow solid. LC-MS purity: 100% (UV at 254 nm), MS: 618.2 [M+H]⁺.

### Step 3-3

### 7-(4-(6-(benzyloxy)-2-phenyl-3,4-dihydronaphthalen-1-yl)-3-methoxyphenyl)-2-(dimethoxymethyl)-7-azaspiro[3.5]nonane

Pd(dppf)Cl₂ (88.4 mg, 0.121 mmol, 0.1 eq), and Na₂CO₃ (384.7 mg, 3.63 mmol, 3.0 eq) was added to a degassed solution of 7-(4-(6-(benzyloxy)-2-bromo-3,4-dihydronaphthalen-1-yl)-3-methoxyphenyl)-2-(dimethoxymethyl)-7-azaspiro[3.5]nonane (750 mg, 1,21 mmol, 1.0 eq) and phenylboronic acid (177 mg, 1,452 mmol, 1,2 eq) in 1,4-dioxane/H₂O (30 mL/3 mL). The reaction mixture was heated to 90° C. for 16 hours. LC-MS showed the reaction was completed. The reaction mixture was cooled to rt, the mixture was diluted with water and washed with EtOAc, the organic phase was dried with Na₂SO₄ and concentrated under vacuum. The residue was purified by SiO₂ column chromatography (EtOAc:PE=1:5) to afford 7-(4-(6-(benzyloxy)-2-phenyl-3,4-dihydronaphthalen-1-yl)-3-methoxyphenyl)-2-(dimethoxymethyl)-7-azaspiro[3.5]nonane (605 mg, 81%) as a yellow oil.

LC-MS purity: 100% (UV at 254 nm), MS: 616.4 [M+H]⁺.

### Step 3-4

### 5-(4-(2-(dimethoxymethyl)-7-azaspiro[3.5]nonan-7-yl)-2-methoxyphenyl)-6-phenyl-5,6,7,8-tetrahydronaphthalen-2-ol

A mixture of 7-(4-(6-(benzyloxy)-2-phenyl-3,4-dihydronaphthalen-1-yl)-3-methoxyphenyl)-2-(dimethoxymethyl)-7-azaspiro[3.5]nonane (605 mg, 0.98 mmol, 1.0 eq) and Pd/C(182 mg) in MeOH (20 mL) was degassed under reduced pressure, purged with H₂ atmosphere, The reaction mixture was heated to 40° C. for 16 hours. LC-MS showed the reaction was completed. The reaction mixture was cooled to room temperature, filtered, the precipitate was washed with MeOH, the filtrate was evaporated, the residue was purified by Chemflash to afford 5-(4-(2-(dimethoxymethyl)-7-azaspiro[3.5]nonan-7-yl)-2-methoxyphenyl)-6-phenyl-5,6,7,8-tetrahydronaphthalen-2-ol (387.7 mg, 74.8%) as a yellow solid.

LC-MS purity: 100% (UV at 254 nm), LC-MS: 528.4 [M+H]⁺.

¹H NMR (400M Hz, DMSO-d6) δ 9.04 (s, 1H), 7.06 (d, J=6.8 Hz, 3H), 6.76-6.70 (m, 2H), 6.55 (dd, J=8.1, 5.3 Hz, 2H), 6.44 (dd, J=8.3, 2.4 Hz, 1H), 6.35 (d, J=8.4 Hz, 1H), 6.29 (dd, J=8.3, 1,1 Hz, 1H), 6.10 (s, 1H), 4.64 (d, J=5.1 Hz, 1H), 4.28 (d, J=7.0 Hz, 1H), 3.20 (s, 8H), 2.97 (d, J=4.2 Hz, 2H), 2.93 (s, 3H), 2.89 (dd, J=11,2, 6.0 Hz, 3H), 2.22-2.11 (m, 1H), 1.78 (dd, J=11.0, 10.1 Hz, 2H), 1.56 (ddd, J=19.8, 9.2, 2.8 Hz, 8H),

### Intermediate 4:5-(4-(4-(dimethoxymethyl)piperidin-1-yl)-5-fluoro-2-methoxyphenyl)-6-phenyl-5,6,7,8-tetrahydronaphthalen-2-ol (two isomers)

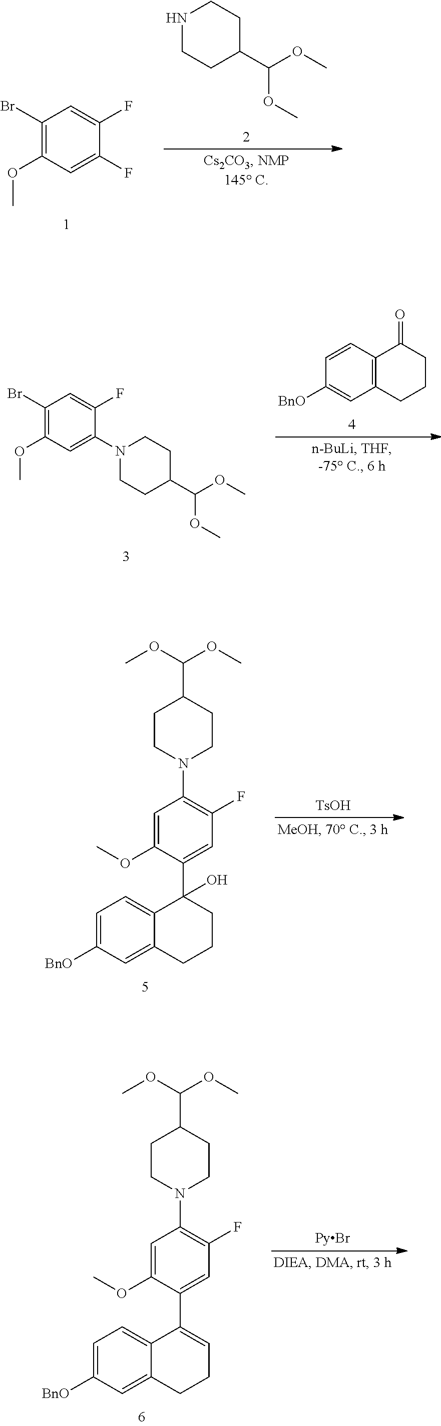

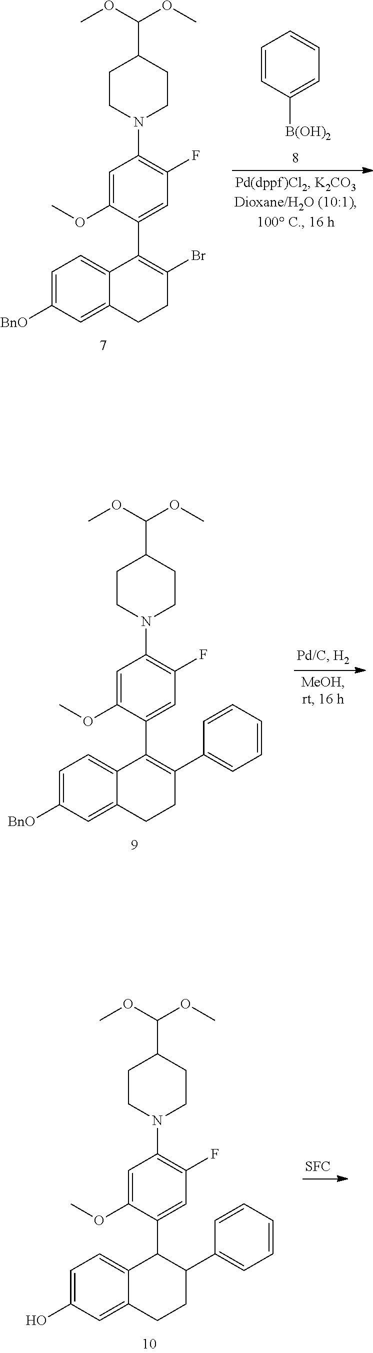 [SMILES: COc1cc(N2CCC(C(OC)OC)CC2)c(F)cc1C1=C(Br)CCc2cc(OCc3ccccc3)ccc21 ; OB(O)c1ccccc1 ; COc1cc(N2CCC(C(OC)OC)CC2)c(F)cc1C1=C(c2ccccc2)CCc2cc(OCc3ccccc3)ccc21 ; COc1cc(N2CCC(C(OC)OC)CC2)c(F)cc1C1c2ccc(O)cc2CCC1c1ccccc1 ; "SFC"]

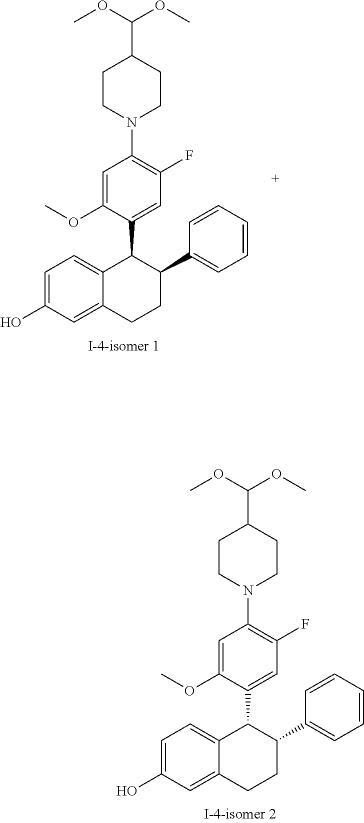 [SMILES: COc1cc(N2CCC(C(OC)OC)CC2)c(F)cc1[C@@H]1c2ccc(O)cc2CC[C@@H]1c1ccccc1 ; COc1cc(N2CCC(C(OC)OC)CC2)c(F)cc1[C@H]1c2ccc(O)cc2CC[C@H]1c1ccccc1]

### Step 1:1-(4-bromo-2-fluoro-5-methoxyphenyl)-4-(dimethoxymethyl)piperidine

To a mixture of 1-bromo-4,5-difluoro-2-methoxybenzene (8.5 g, 38.1 mmol, 1 eq.) and 4-(dimethoxymethyl)piperidine (6.1 g, 38.1 mmol, 1 eq.) in NMP (50 mL) was added Cs₂CO₃ (37.3 g, 114 mmol, 3 eq.). The mixture was purged with nitrogen and stirred at 145° C. overnight. The mixture was cooled to room temperature and then poured into H₂O (500 mL). The mixture was extracted with EtOAc (150 mL). The organic phase was dried over Na₂SO₄ and concentrated. The residue was purified by column chromatography on silica gel eluted with 0-20% EtOAc/hexane to afford 1-(4-bromo-2-fluoro-5-methoxyphenyl)-4-(dimethoxymethyl)piperidine as yellow oil. (2.5 g, 18% yield).

LC-MS purity: 100% (UV at 254 nm), 362.1/364.1 [M+H]⁺.

### Step 2:6-(benzyloxy)-1-(4-(4-(dimethoxymethyl)piperidin-1-yl)-5-fluoro-2-methoxyphenyl)-1,2,3,4-tetrahydronaphthalen-1-ol

To a mixture of 1-(4-bromo-2-fluoro-5-methoxyphenyl)-4-(dimethoxymethyl)piperidine (5.0 g, 13.8 mmol, 1 eq.) in dry THF (25 mL) under Argon was added dropwise n-BuLi (2.50 M, 6.6 mL, 1,2 eq.). The mixture was stirred at −75° C. for 1.5h, and 6-(benzyloxy)-3,4-dihydronaphthalen-1 (2H)-one (4.2 g, 16.6 mmol, 1,1 eq.) in dry THF (10 mL) was added dropwise. The mixture was stirred at −75° C. for 3 h. The mixture was quenched by the addition of the saturated aqueous NH₄Cl. The mixture was poured into H₂O (40 mL) and extracted with EtOAc (2×30 mL). The combined organic layer was washed with brine (40 mL), dried over Na₂SO₄, filtered and concentrated. The residue was purified by column chromatography on silica gel eluted with 0-30% EtOAc/hexane to afford 6-(benzyloxy)-1-(4-(4-(dimethoxymethyl)piperidin-1-yl)-5-fluoro-2-methoxyphenyl)-1,2,3,4-tetrahydronaphthalen-1-ol (2.0 g, 26.8% yield) as white solid. LC-MS purity: 100% (UV at 254 nm), 536.1 [M+H]⁺.

### Step 3:1-(4-(6-(benzyloxy)-3,4-dihydronaphthalen-1-yl)-2-fluoro-5-methoxyphenyl)-4-(dimethoxymethyl)piperidine

To a mixture of 6-(benzyloxy)-1-(4-(4-(dimethoxymethyl)piperidin-1-yl)-5-fluoro-2-methoxyphenyl)-1,2,3,4-tetrahydronaphthalen-1-ol (2.5 g, 4.7 mmol, 1 eq.) in MeOH (8 mL) was added TsOH (171 mg, 0.9 mmol, 0.2 eq.). The mixture was stirred at 70° C. for 3 h and concentrated. The residue was purified by column chromatography on silica gel eluted with 0-30% EtOAc/hexane to afford 1-(4-(6-(benzyloxy)-3,4-dihydronaphthalen-1-yl)-2-fluoro-5-methoxyphenyl)-4-(dimethoxymethyl)piperidine (1.6 g, 65.7% yield) as yellow solid. LC-MS purity: 100% (UV at 254 nm), 518.3 [M+H]⁺.

### Step 4:1-(4-(6-(benzyloxy)-2-bromo-3,4-dihydronaphthalen-1-yl)-2-fluoro-5-methoxyphenyl)-4-(dimethoxymethyl)piperidine

To a mixture of 1-(4-(6-(benzyloxy)-3,4-dihydronaphthalen-1-yl)-2-fluoro-5-methoxyphenyl)-4-(dimethoxymethyl)piperidine (1.6 g, 3.1 mmol, 1 eq.) and DIEA (0.8 g, 6.2 mmol, 2 eq.) in DMA (10 mL), was added pyridinium tribromide (1,2 g, 3.7 mmol, 1,2 eq.) at 0° C. The mixture was stirred at room temperature for 3 h. The mixture was poured into H₂O (50 mL) and extracted with EtOAc (2×20 mL). The combined organic layer was washed with brine (40 mL), dried over Na₂SO₄, filtered and concentrated. The residue was purified by column chromatography on silica gel eluted with 0-20% EtOAc/hexane to afford 1-(4-(6-(benzyloxy)-2-bromo-3,4-dihydronaphthalen-1-yl)-2-fluoro-5-methoxyphenyl)-4-(dimethoxymethyl)piperidine (1.6 g, 86.6% yield) as yellow solid.

LC-MS purity: 100% (UV at 254 nm), 598.2 [M+H]⁺.

### Steps 5:1-(4-(6-(benzyloxy)-2-phenyl-3,4-dihydronaphthalen-1-yl)-2-fluoro-5-methoxyphenyl)-4-(dimethoxymethyl)piperidine

To a mixture of 1-(4-(6-(benzyloxy)-2-bromo-3,4-dihydronaphthalen-1-yl)-2-fluoro-5-methoxyphenyl)-4-(dimethoxymethyl)piperidine (1.8 g, 3.0 mmol, 1 eq.) in dioxane (16 mL) and H₂O (2 mL), was added phenylboronic acid (522 mg, 4.5 mmol, 1.5 eq.). K₂CO₃ (636 mg, 3 mmol, 2 eq.) followed by Pd(dppf)Cl₂ (137 mg, 0.15 mmol, 0.05 eq.). The mixture was stirred at 100° C. for 16 hours under Argon. The mixture was cooled to room temperature, poured into H₂O (50 mL) and extracted with EtOAc (2×20 mL). The combined organic layer was washed with brine (40 mL), dried over Na₂SO₄, filtered and concentrated. The residue was purified by column chromatography on silica gel eluted with 0-20% EtOAc/hexane to afford 1-(4-(6-(benzyloxy)-2-phenyl-3,4-dihydronaphthalen-1-yl)-2-fluoro-5-methoxyphenyl)-4-(dimethoxymethyl)piperidine (1.3 g, 72.2% yield) as yellow solid.

LC-MS purity: 100% (UV at 254 nm), 594.3 [M+H]⁺.

### Steps 6:5-(4-(4-(dimethoxymethyl)piperidin-1-yl)-5-fluoro-2-methoxyphenyl)-6-phenyl-5,6,7,8-tetrahydronaphthalen-2-ol

To a mixture of 1-(4-(6-(benzyloxy)-2-phenyl-3,4-dihydronaphthalen-1-yl)-2-fluoro-5-methoxyphenyl)-4-(dimethoxymethyl)piperidine (440 mg, 0.7 mmol, 1 eq) in MeOH (10 mL) was added Pd/C(100 mg, 10% on Carbon, wetted with c.a,55% water). The mixture was stirred at room temperature overnight under H₂. The catalyst was removed by filtration and the filtrate was concentrated to afford 5-(4-(4-(dimethoxymethyl)piperidin-1-yl)-5-fluoro-2-methoxyphenyl)-6-phenyl-5,6,7,8-tetrahydronaphthalen-2-ol (200 mg, 53.3% yield) as white solid.

LC-MS purity: 68.0% (UV at 254 nm), 506.5 [M+H]⁺.

### Steps 7: (55,6S)-5-(4-(4-(dimethoxymethyl)piperidin-1-yl)-5-fluoro-2-methoxyphenyl)-6-phenyl-5,6,7,8-tetrahydronaphthalen-2-ol & (5R,6R)-5-(4-(4-(dimethoxymethyl)piperidin-1-yl)-5-fluoro-2-methoxyphenyl)-6-phenyl-5,6,7,8-tetrahydronaphthalen-2-ol

5-(4-(4-(dimethoxymethyl)piperidin-1-yl)-5-fluoro-2-methoxyphenyl)-6-phenyl-5,6,7,8-tetrahydronaphthalen-2-ol (200 mg, 0.4 mmol) was separated by SFC to afford (55,6S)-5-(4-(4-(dimethoxymethyl)piperidin-1-yl)-5-fluoro-2-methoxyphenyl)-6-phenyl-5,6,7,8-tetrahydronaphthalen-2-ol (90 mg) and (5R,6R)-5-(4-(4-(dimethoxymethyl)piperidin-1-yl)-5-fluoro-2-methoxyphenyl)-6-phenyl-5,6,7,8-tetrahydronaphthalen-2-ol (90 mg) with both structures being tentatively assigned.

### Intermediate 5:7-(4-((1R,2S)-6-hydroxy-2-phenyl-1,2,3,4-tetrahydronaphthalen-1-yl)phenyl)-7-azaspiro[3.5]nonane-2-carbaldehyde

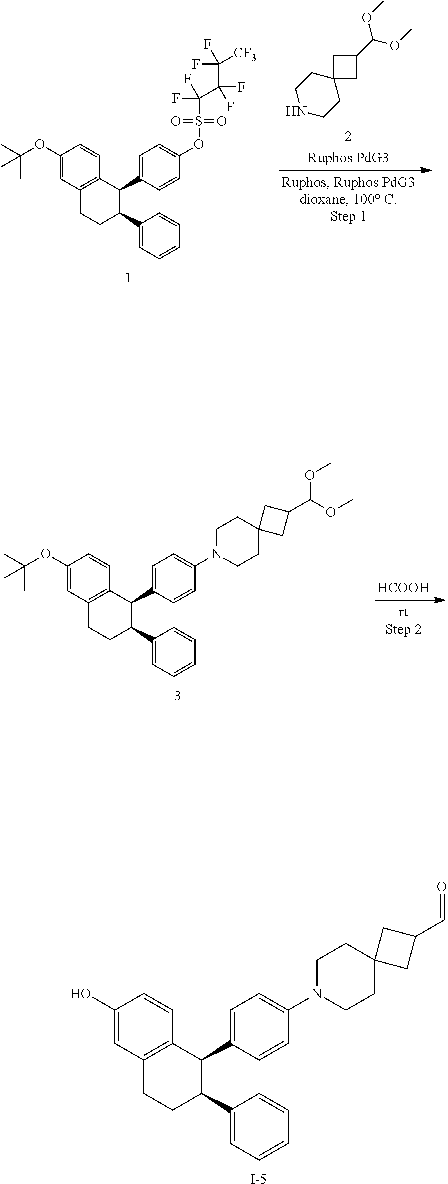

### Step 5-1

### 7-(4-((1R,2S)-6-(tert-butoxy)-2-phenyl-1,2,3,4-tetrahydronaphthalen-1-yl)phenyl)-2-(dimethoxymethyl)-7-azaspiro[3.5]nonane

To a mixture of 4-((1R,2S)-6-(tert-butoxy)-2-phenyl-1,2,3,4-tetrahydronaphthalen-1-yl) phenyl 1,1,2,2,3,3,4,4,4-nonafluorobutane-1-sulfonate (100 mg, 0.15 mmol, 1.0 eq), 2-(dimethoxymethyl)-7-azaspiro[3.5]nonane (30 mg, 0.15 mmol, 1.0 eq), t-BuONa (44 mg, 0.46 mmol, 3.0 eq) and Ruphos (7 mg, 0.02 mmol, 0.1 eq) in 1,4-dioxane (5 mL) was added Ruphos PdG₃ (13 mg, 0.02 mmol, 0.1 eq), then stirred at 100° C. for 16 hours. LC-MS showed the reaction was completed. The reaction mixture was cooled to rt, the mixture was diluted with water and washed with EtOAc, the organic phase was dried with Na₂SO₄ and concentrated under vacuum. the residue was purified by SiO₂ column chromatography (EtOAc:PE=1:20) to afford 7-(4-((1R,2S)-6-(tert-butoxy)-2-phenyl-1,2,3,4-tetrahydronaphthalen-1-yl)phenyl)-2-(dimethoxymethyl)-7-azaspiro[3.5]nonane (26 mg, 31%) as a yellow oil.

LC-MS purity: 62.1% (UV at 254 nm), LC-MS: 554.3 [M+H]⁺.

### Step 5-2

### 7-(4-((1R,2S)-6-hydroxy-2-phenyl-1,2,3,4-tetrahydronaphthalen-1-yl)phenyl)-7-azaspiro[3.5]nonane-2-carbaldehyde

To a mixture of 7-(4-((1R,2S)-6-(tert-butoxy)-2-phenyl-1,2,3,4-tetrahydronaphthalen-1-yl)phenyl)-2-(dimethoxymethyl)-7-azaspiro[3.5]nonane (26 mg, 0.04 mmol, 1 eq) in HCOOH (3 mL) was stirred at 25° C. for 16 hours. The mixture was concentrated to give crude product 7-(4-((1R,2S)-6-hydroxy-2-phenyl-1,2,3,4-tetrahydronaphthalen-1-yl)phenyl)-7-azaspiro[3.5]nonane-2-carbaldehyde (13 mg, 69%) as a colorless liquid.

LC-MS purity: 100% (UV at 254 nm), LC-MS: 452.2 [M+H]⁺.

### Intermediate 6: (R)-N-((S)-2,6-dioxopiperidin-3-yl)-1,2,3,4,4a,5-hexahydropyrazino[1,2-d]pyrido[2,3-b][1,4]oxazine-8-carboxamide hydrochloride salt

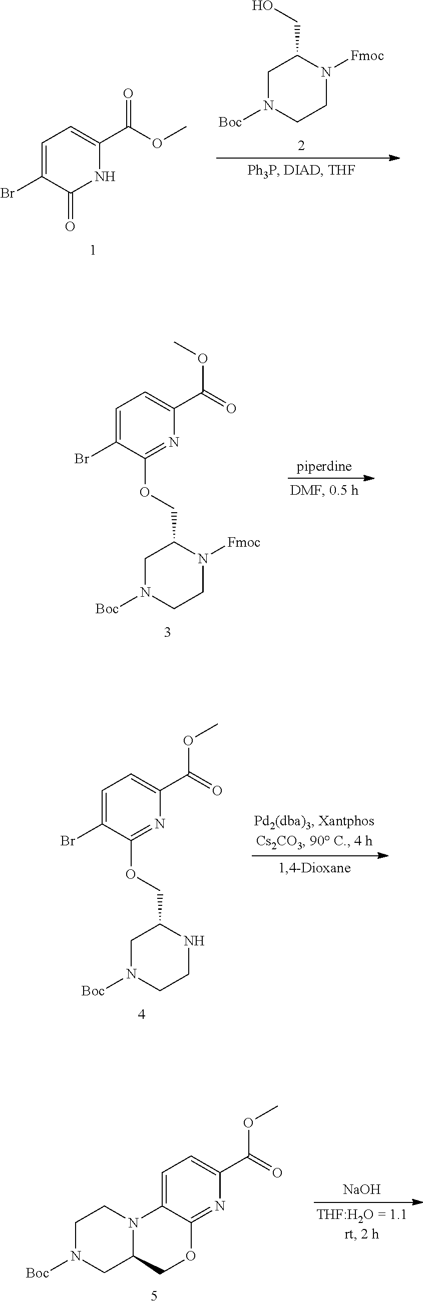

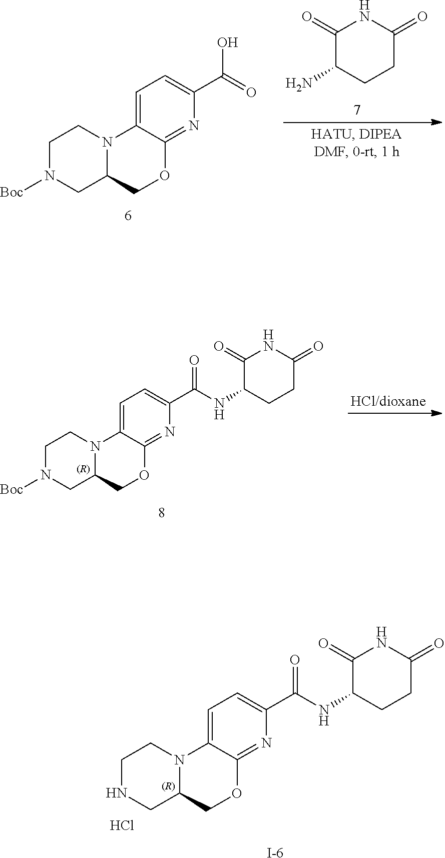

### Step 1: (R)-1-((9H-fluoren-9-yl)methyl) 4-tert-butyl 2-(((3-bromo-6-(methoxycarbonyl)pyridin-2-yl)oxy)methyl)piperazine-1,4-dicarboxylate

To a mixture of methyl 5-bromo-6-oxo-1,6-dihydropyridine-2-carboxylate (2.5 g, 10.7 mmol, 1 eq.) in THF (50 mL) was added (R)-1-((9H-fluoren-9-yl)methyl) 4-tert-butyl 2-(hydroxymethyl)piperazine-1,4-dicarboxylate (5.7 g, 12.9 mmol, 1,2 eq.) and PPh₃ (8.4 g, 32.1 mmol, 3 eq.) and the mixture was stirred at 60° C. To the mixture was added DIAD (6.5 g, 32.1 mmol, 3 eq.) dropwise and the mixture was stirred at room temperature for 12 h. The mixture was evaporated in vacuo and the residue was purified by column chromatography on silica gel eluted with 0-30% EtOAc/hexane to afford (R)-1-((9H-fluoren-9-yl)methyl) 4-tert-butyl 2-(((3-bromo-6-(methoxycarbonyl)pyridin-2-yl)oxy)methyl)piperazine-1,4-dicarboxylate (5.0 g, 70% yield) as yellow solid.

### Step 2: (R)-tert-butyl 3-(((3-bromo-6-(methoxycarbonyl)pyridin-2-yl)oxy)methyl)piperazine-1-carboxylate

To a mixture of (R)-1-((9H-fluoren-9-yl)methyl) 4-tert-butyl 2-(((3-bromo-6-(methoxycarbonyl)pyridin-2-yl)oxy)methyl)piperazine-1,4-dicarboxylate (5 g, 7.6 mmol 1 eq.) in DMF (50 mL) was added piperidine (1,1 g, 15.2 mmol, 2 eq.). The mixture was stirred at room temperature for 1 h, diluted with ethyl acetate (100 mL) and washed with water (50 mL). The organic phase was washed with brine, dried over Na₂SO₄ and filtered. The filtrate was evaporated in vacuo. The crude was purified by column chromatography on silica gel eluted with 0-5% DCM in methanol to give (R)-tert-butyl 3-(((3-bromo-6-(methoxycarbonyl)pyridin-2-yl)oxy)methyl)piperazine-1-carboxylate (2.4 g, 75% yield). LC-MS purity: 100% (UV at 254 nm), ms: 430.2 [M+1]⁺.

### Step 3: (R)-3-tert-butyl 8-methyl 1,2,4a,5-tetrahydropyrazino[1,2-d]pyrido[2,3-b][1,4]oxazine-3,8 (4H)-dicarboxylate

To a mixture of (R)-tert-butyl 3-(((3-bromo-6-(methoxycarbonyl)pyridin-2-yl)oxy)methyl)piperazine-1-carboxylate (2.4 g, 5,6 mmol, 1 eq.), XantPhos (486 mg, 0.84 mmol, 0.15 eq.), and Cs₂CO₃ (5.4 g, 16.8 mmol, 3 eq.) in dioxane (50 mL) was added Pd₂ (dba) 3 (511 mg, 0.56 mmol, 0.1 eq.) under Ar flow and the mixture was stirred at 100° C. for 16 h. The mixture was diluted with ethyl acetate (100 mL) and washed with water (50 mL). The organic phase was washed with brine, dried over Na₂SO₄ and filtered. The filtrate was evaporated in vacuo and the residue was purified by column chromatography on silica gel eluted with using 0-50% EtOAc/hexane to give (R)-3-tert-butyl 8-methyl 1,2,4a,5-tetrahydropyrazino[1,2-d]pyrido[2,3-b][1,4]oxazine-3.8 (4H)-dicarboxylate (1.3 g, 68% yield) as white solid. LC-MS purity: 100% (UV at 254 nm), 350.4 [M+H]⁺.

### Step 4: (R)-3-(tert-butoxycarbonyl)-1,2,3,4,4a,5-hexahydropyrazino[1,2-d]pyrido[2,3-b][1,4]oxazine-8-carboxylic acid

To a mixture of (R)-3-tert-butyl 8-methyl 1,2,4a,5-tetrahydropyrazino[1,2-d]pyrido[2,3-b][1,4]oxazine-3.8 (4H)-dicarboxylate (1.3 g, 3.7 mmol, 1 eq.) in THF (10 mL) and water (10 mL) was added sodium hydroxide (590 mg, 14.8 mmol, 4 eq) and the mixture was stirred at room temperature for 2 h. The mixture was adjusted to pH 5-6 with aq. HCl (1 M) and extracted with ethyl acetate (20 mL). The organic layer was washed with brine, dried over sodium sulfate and filtered. The filtrate was evaporated to afford (R)-3-(tert-butoxycarbonyl)-1,2,3,4,4a,5-hexahydropyrazino[1,2-d]pyrido[2,3-b][1,4]oxazine-8-carboxylic acid (1.3 g, crude) as white solid. LC-MS purity: 100% (UV at 254 nm), 336.3 [M+H]⁺.

### Step 5: tert-butyl (R)-8-(((S)-2,6-dioxopiperidin-3-yl) carbamoyl)-1,2,4a,5-tetrahydropyrazino[1,2-d]pyrido[2,3-b][1,4]oxazine-3 (4H)-carboxylate

To a mixture of (R)-3-(tert-butoxycarbonyl)-1,2,3,4,4a,5-hexahydropyrazino[1,2-d]pyrido[2,3-b][1,4]oxazine-8-carboxylic acid (1.3 g, 3.8 mmol, 1 eq) in DMF (10 mL) was added HATU (1.7 g, 4.6 mmol, 1,2 eq) and DIPEA (980 mg, 7.6 mmol, 2 eq) and the mixture was stirred at room temperature for 1 h. The mixture was purified directly by reverse phase column chromatography (0-90% acetonitrile/0.05% formic acid)) to afford (R)-tert-butyl 8-(((S)-2,6-dioxopiperidin-3-yl) carbamoyl)-1,2,4a,5-tetrahydropyrazino[1,2-d]pyrido[2,3-b][1,4]oxazine-3 (4H)-carboxylate (1.3 g, 76% yield) as white solid. LC-MS purity: 100% (UV at 254 nm), 446.2 [M+H]⁺.

### Step 6: (R)-N-((S)-2,6-dioxopiperidin-3-yl)-1,2,3,4,4a,5-hexahydropyrazino[1,2-d]pyrido[2,3-b][1,4]oxazine-8-carboxamide hydrochloride

A mixture of (R)-tert-butyl 8-(((S)-2,6-dioxopiperidin-3-yl) carbamoyl)-1,2,4a,5-tetrahydropyrazino[1,2-d]pyrido[2,3-b][1,4]oxazine-3 (4H)-carboxylate (1.3 g, 2.9 mmol, 1 eq.) in HCl/dioxane (10 mL) was stirred at room temperature for 2 h. The reaction mixture was concentrated to afford (R)-N-((S)-2,6-dioxopiperidin-3-yl)-1,2,3,4,4a,5-hexahydropyrazino[1,2-d]pyrido[2,3-b][1,4]oxazine-8-carboxamide hydrochloride (1.0 g, 91% yield) as white solid.

LC-MS purity: 100% (UV at 254 nm), ms: 346.2 [M+1]⁺.

¹H NMR (400 MHZ, DMSO): δ 10.84 (s, 1H), 9.63-9.33 (m, 2H), 8.56 (d, J=8.4 Hz, 1H), 7.62 (d, J=8.2 Hz, 1H), 7.44 (d, J=8.4 Hz, 1H), 4.77-4.70 (m, 1H), 4.51-4.49 (m, 2H), 4.20-4.05 (m, 2H), 3.66-3.55 (m, 1H), 3.47-3.39 (m, 2H), 3.22-2.98 (m, 2H), 2.89-2.67 (m, 2H), 2.26-2.11 (m, 1H), 2.02-1.90 (m, 1H).

### Intermediate 7: (S)-N-((S)-2,6-dioxopiperidin-3-yl)-1,2,3,4,4a,5-hexahydropyrazino[1,2-d]pyrido[2,3-b][1,4]oxazine-8-carboxamide hydrochloride salt

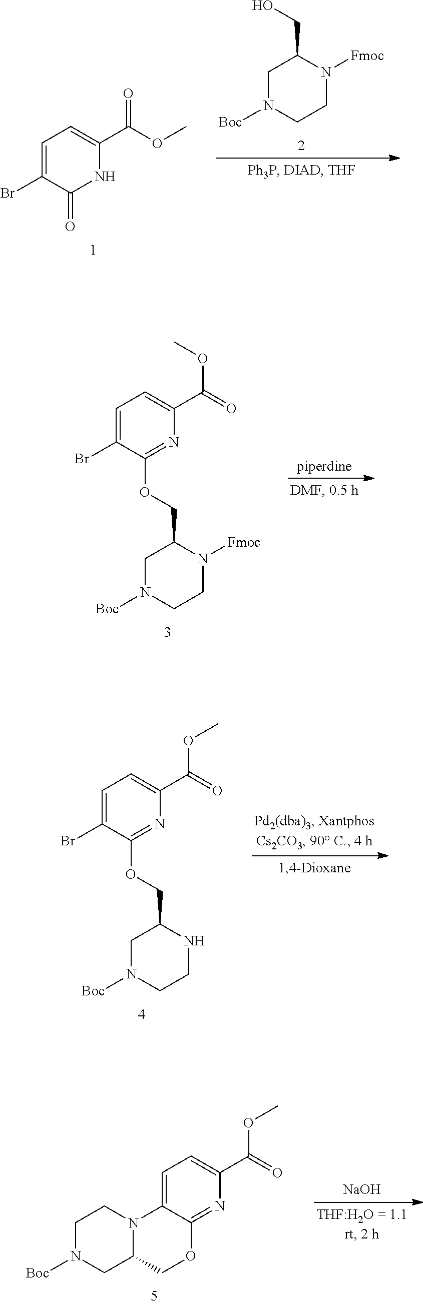

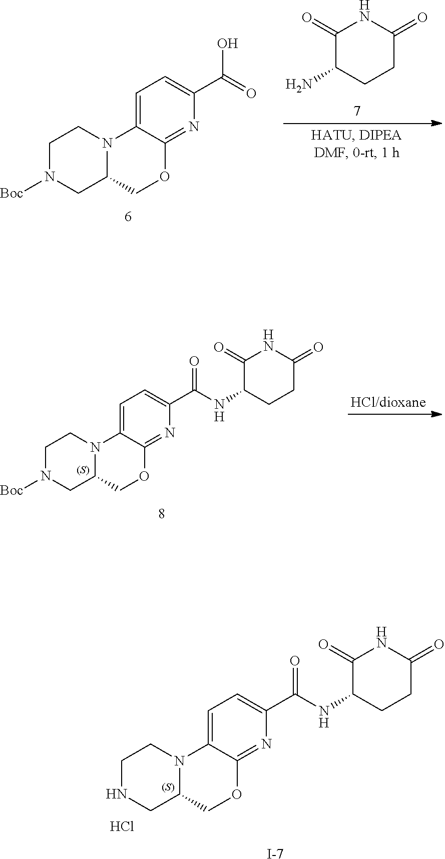

### Step 1: (S)-1-((9H-fluoren-9-yl)methyl) 4-tert-butyl 2-(((3-bromo-6-(methoxycarbonyl)pyridin-2-yl)oxy)methyl)piperazine-1,4-dicarboxylate

To a mixture of methyl 5-bromo-6-oxo-1,6-dihydropyridine-2-carboxylate (2.5 g, 10.7 mmol, 1 eq.) in THF (50 mL) was added (R)-1-((9H-fluoren-9-yl)methyl) 4-tert-butyl 2-(hydroxymethyl)piperazine-1,4-dicarboxylate (5.7 g, 12.9 mmol, 1,2 eq.) and PPh₃ (8.4 g, 32.1 mmol, 3 eq.) and the mixture was stirred at 60° C. To the mixture was added DIAD (6.5 g, 32.1 mmol, 3 eq.) dropwise and the mixture was stirred at room temperature for 12 h. The mixture was evaporated in vacuo and the residue was purified by column chromatography on silica gel eluted with 0-30% EtOAc/hexane to afford (R)-1-((9H-fluoren-9-yl)methyl) 4-tert-butyl 2-(((3-bromo-6-(methoxycarbonyl)pyridin-2-yl)oxy)methyl)piperazine-1,4-dicarboxylate (3.0 g, 50% yield) as yellow solid.

### Step 2: tert-butyl(S)-3-(((3-bromo-6-(methoxycarbonyl)pyridin-2-yl)oxy)methyl)piperazine-1-carboxylate

To a mixture of (R)-1-((9H-fluoren-9-yl)methyl) 4-tert-butyl 2-(((3-bromo-6-(methoxycarbonyl)pyridin-2-yl)oxy)methyl)piperazine-1,4-dicarboxylate (3 g, 5,6 mmol 1 eq.) in DMF (50 mL) was added piperidine (1,1 g, 15.2 mmol, 3 eq.). The mixture was stirred at room temperature for 1 h, diluted with ethyl acetate (100 mL) and washed with water (50 mL). The organic phase was washed with brine, dried over Na₂SO₄ and filtered. The filtrate was evaporated in vacuo and the residue was purified by column chromatography on silica gel eluted with 0-5% DCM in methanol to give (R)-tert-butyl 3-(((3-bromo-6-(methoxycarbonyl)pyridin-2-yl)oxy)methyl)piperazine-1-carboxylate (2.4 g, 95% yield). LC-MS purity: 100% (UV at 254 nm), ms: 430.2 [M+H]⁺.

### Step 3: (S)-3-tert-butyl 8-methyl 1,2,4a,5-tetrahydropyrazino[1,2-d]pyrido[2,3-b][1,4]oxazine-3,8 (4H)-dicarboxylate

To a mixture of (R)-tert-butyl 3-(((3-bromo-6-(methoxycarbonyl)pyridin-2-yl)oxy)methyl)piperazine-1-carboxylate (2.4 g, 5,6 mmol, 1 eq.), XantPhos (486 mg, 0.84 mmol, 0.15 eq.), and Cs₂CO₃ (5.4 g, 16.8 mmol, 3 eq.) in dioxane (50 mL) was added Pd₂ (dba) 3 (511 mg, 0.56 mmol, 0.1 eq.) under Ar flow and the mixture was stirred at 100° C. for 16 h. The mixture was diluted with ethyl acetate (100 mL) and washed with water (50 mL). The organic phase was washed with brine, dried over Na₂SO₄ and filtered. The filtrate was evaporated in vacuo and the residue was purified by column chromatography on silica gel eluted with 0-50% EtOAc/hexane to give (R)-3-tert-butyl 8-methyl 1,2,4a,5-tetrahydropyrazino[1,2-d]pyrido[2,3-b][1,4]oxazine-3,8 (4H)-dicarboxylate (1.3 g, 68% yield) as white solid. LC-MS purity: 100% (UV at 254 nm), ms: 350.4 [M+H]⁺.

### Step 4: (S)-3-(tert-butoxycarbonyl)-1,2,3,4,4a,5-hexahydropyrazino[1,2-d]pyrido[2,3-b][1,4]oxazine-8-carboxylic acid

To a mixture of (R)-3-tert-butyl 8-methyl 1,2,4a,5-tetrahydropyrazino[1,2-d]pyrido[2,3-b][1,4]oxazine-3.8 (4H)-dicarboxylate (1.3 g, 3.7 mmol, 1 eq.) in THF (10 mL) and water (10 mL) was added sodium hydroxide (590 mg, 14.8 mmol, 4 eq) and the mixture was stirred at room temperature for 2 h. The mixture was adjusted to pH 5-6 with aq. HCl (1 M) and extracted with ethyl acetate (20 mL). The organic layer was washed with brine, dried over sodium sulfate and filtered. The filtrate was evaporated to afford (R)-3-(tert-butoxycarbonyl)-1,2,3,4,4a,5-hexahydropyrazino[1,2-d]pyrido[2,3-b][1,4]oxazine-8-carboxylic acid (1.3 g, crude) as white solid. LC-MS purity: 100% (UV at 254 nm), 336.3 [M+H]⁺.

### Steps 5: (S)-tert-butyl 8-(((S)-2,6-dioxopiperidin-3-yl) carbamoyl)-1,2,4a,5-tetrahydropyrazino[1,2-d]pyrido[2,3-b][1,4]oxuzine-3 (4H)-carboxylate

To a mixture of(S)-3-(tert-butoxycarbonyl)-1,2,3,4,4a,5-hexahydropyrazino[1,2-d]pyrido[2,3-b][1,4]oxazine-8-carboxylic acid (880 mg, 2.6 mmol, 1 eq.) in DMF (10 mL) was added T₃P (3.2 mL, 5.2 mmol, 2 eq.) and DIPEA (0.64 mL, 5.2 mmol, 2 eq). The mixture was stirred at room temperature for 1 h, quenched with water (10 mL) and purified directly by reverse phase column chromatography (0-90% Acetonitrile/0.05% Formic acid)) to afford (S)-tert-butyl 8-(((S)-2,6-dioxopiperidin-3-yl) carbamoyl)-1,2,4a,5-tetrahydropyrazino[1,2-d]pyrido[2,3-b][1,4]oxazine-3 (4H)-carboxylate (620 mg, 76% yield) as a white solid. LC-MS purity: 100% (UV at 254 nm), ms: 446.2 [M+H]⁺.

### Steps 6: (S)-N-((S)-2,6-dioxopiperidin-3-yl)-1,2,3,4,4a,5-hexahydropyrazino[1,2-d]pyrido[2,3-b][1,4]oxazine-8-carboxamide hydrochloride

A mixture of (R)-tert-butyl 8-(((S)-2,6-dioxopiperidin-3-yl) carbamoyl)-1,2,4a,5-tetrahydropyrazino[1,2-d]pyrido[2,3-b][1,4]oxazine-3 (4H)-carboxylate (620 mg, 1.4 mmol, 1 eq.) in HCl/dioxane (10 mL) was stirred at room temperature for 2 h. The reaction mixture was concentrated to afford (R)-N-((S)-2,6-dioxopiperidin-3-yl)-1,2,3,4,4a,5-hexahydropyrazino[1,2-d]pyrido[2,3-b][1,4]oxazine-8-carboxamide hydrochloride (520 mg, crude) as white solid. LC-MS purity: 100% (UV at 254 nm), 346.2 [M+H]⁺.

¹H NMR (400 MHZ, DMSO): δ 10.84 (s, 1H), 9.63-9.33 (m, 2H), 8.56 (d, J=8.4 Hz, 1H), 7.62 (d, J=8.2 Hz, 1H), 7.44 (d, J=8.4 Hz, 1H), 4.77-4.70 (m, 1H), 4.51-4,49 (m, 2H), 4.20-4.05 (m, 2H), 3.66-3.55 (m, 1H), 3.47-3.39 (m, 2H), 3.22-2.98 (m, 2H), 2.89-2.67 (m, 2H), 2.26-2.11 (m, 1H), 2.02-1.90 (m, 1H),

### Intermediate 8:3-((4-(piperidin-4-yl)phenyl)amino)piperidine-2,6-dione hydrochloride salt

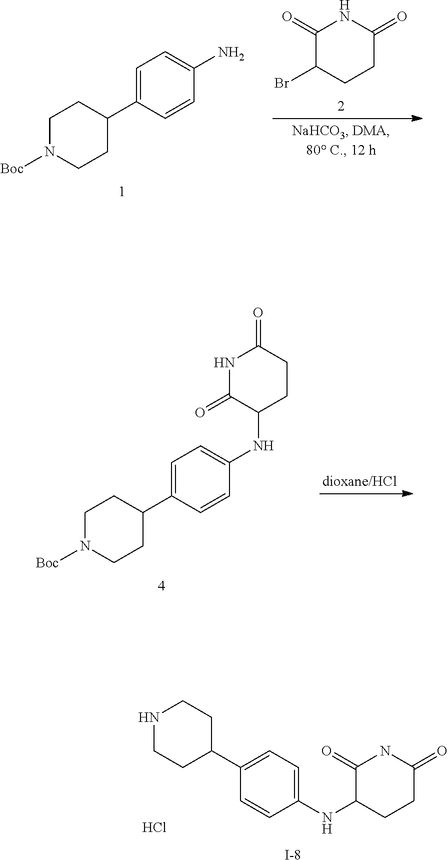

### Step 1: tert-butyl 4-(4-((2,6-dioxopiperidin-3-yl)amino)phenyl)piperidine-1-carboxylate

To a mixture of tert-butyl 4-(4-aminophenyl)piperidine-1-carboxylate (1.5 g, 5.4 mmol 1.0 eq.) in DMA (8 mL) was added 3-bromopiperidine-2,6-dione (1.0 g, 5.428 mmol 1.0 eq) and NaHCO₃ (456 mg, 5.4 mmol 1.0 eq.). The mixture was stirred at 80° C. overnight and cooled to room temperature. The mixture was concentrated and the residue was purified by column chromatography on silica gel eluted with 0-100% EtOAc/hexane to afford tert-butyl 4-(4-((2,6-dioxopiperidin-3-yl)amino)phenyl)piperidine-1-carboxylate as light blue solid (1.6 g, 76.0% yield). LC-MS purity: 100% (UV at 254 nm), 388.0 [M+H]⁺.

### Step 2:3-((4-(piperidin-4-yl)phenyl)amino) piperidine-2,6-dione hydrochloride salt

A mixture of tert-butyl 4-(4-((2,6-dioxopiperidin-3-yl)amino)phenyl)piperidine-1-carboxylate (1.6 g, 4.1 mmol, 1.0 eq.) in HCl/dioxane (10 mL) was stirred at room temperature for 2 h. The mixture was concentrated to afford 3-((4-(piperidin-4-yl)phenyl)amino) piperidine-2,6-dione (1.5 g, crude),. LC-MS purity: 100% (UV at 254 nm), 288.0 [M+H]⁺, ¹H NMR (400 MHZ, DMSO) δ 10.83 (s, 1H), 9.11-8.76 (m, 2H), 6.98 (d, J=8.4 Hz, 2H), 6.72 (d, J=8.4 Hz, 2H), 4.33 (dd, J=11.6, 4.8 Hz, 1H), 3.35-3,25 (m, 2H), 3.00-2.84 (m, 2H), 2.79-2.56 (m, 3H), 2.15-2.01 (m, 1H), 1.92-1.73 (m, 5H),

### Intermediate 9: (R or S)-3-((4-(piperidin-4-yl)phenyl)amino) piperidine-2,6-dione hydrochloride salt

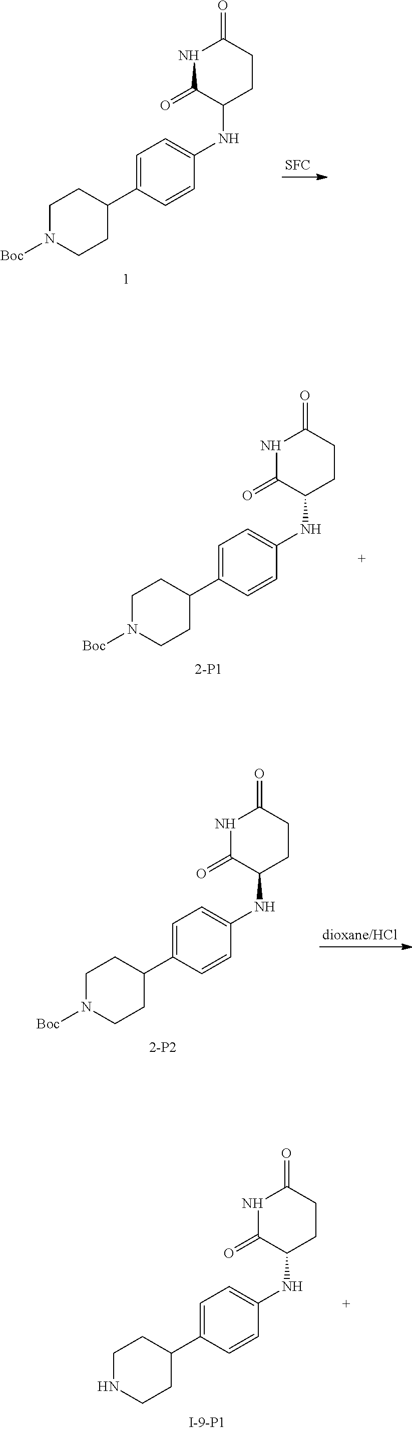

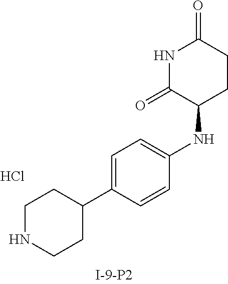 [SMILES: Cl ; O=C1CC[C@@H](Nc2ccc(C3CCNCC3)cc2)C(=O)N1]

### Step 1: (R/S)-tert-butyl 4-(4-((2,6-dioxopiperidin-3-yl)amino)phenyl)piperidine-1-carboxylate

tert-Butyl 4-(4-((2,6-dioxopiperidin-3-yl)amino)phenyl)piperidine-1-carboxylate (1.9 g, 5 mmol) was purified via SFC to afford (R/S)-tert-butyl 4-(4-((2,6-dioxopiperidin-3-yl)amino)phenyl)piperidine-1-carboxylate (P1:450 mg, P2:480 mg), LC-MS purity: 100% (UV at 254 nm), 388.0 [M+H]⁺.

### Step 2: (R/S)-3-((4-(piperidin-4-yl)phenyl)amino) piperidine-2,6-dione hydrochloride salt

A mixture of (R/S)-tert-butyl 4-(4-((2,6-dioxopiperidin-3-yl)amino)phenyl)piperidine-1-carboxylate (100 mg, 0.25 mmol, 1.0 eq) in HCl/dioxane (2 mL) was stirred at room temperature for 2 h. The mixture was concentrated to afford (R/S)-3-((4-(piperidin-4-yl)phenyl)amino) piperidine-2,6-dione hydrochloride salt (90 mg, 100% crude yield),. LC-MS purity: 100% (UV at 254 nm), 288.0 [M+H]⁺, ¹H NMR (400 MHZ, DMSO) δ 10.83 (s, 1H), 9.11-8.76 (m, 2H), 6.98 (d, J=8.4 Hz, 2H), 6.72 (d, J=8.4 Hz, 2H), 4.33 (dd, J=11.6, 4.8 Hz, 1H), 3.35-3,25 (m, 2H), 3.00-2.84 (m, 2H), 2.79-2.56 (m, 3H), 2.15-2.01 (m, 1H), 1.92-1.73 (m, 5H).

### Intermediate 10:3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione hydrochloride salt

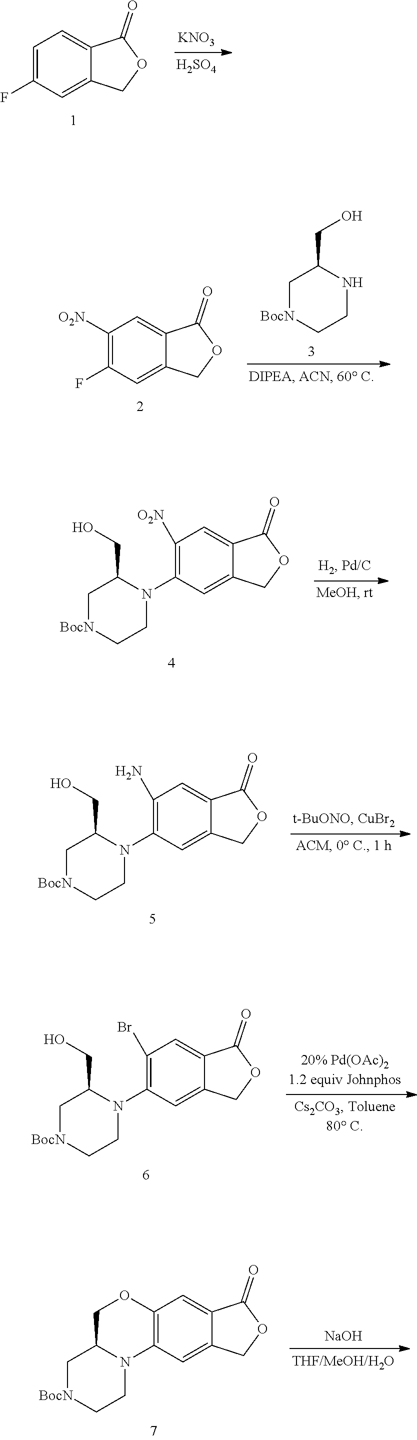

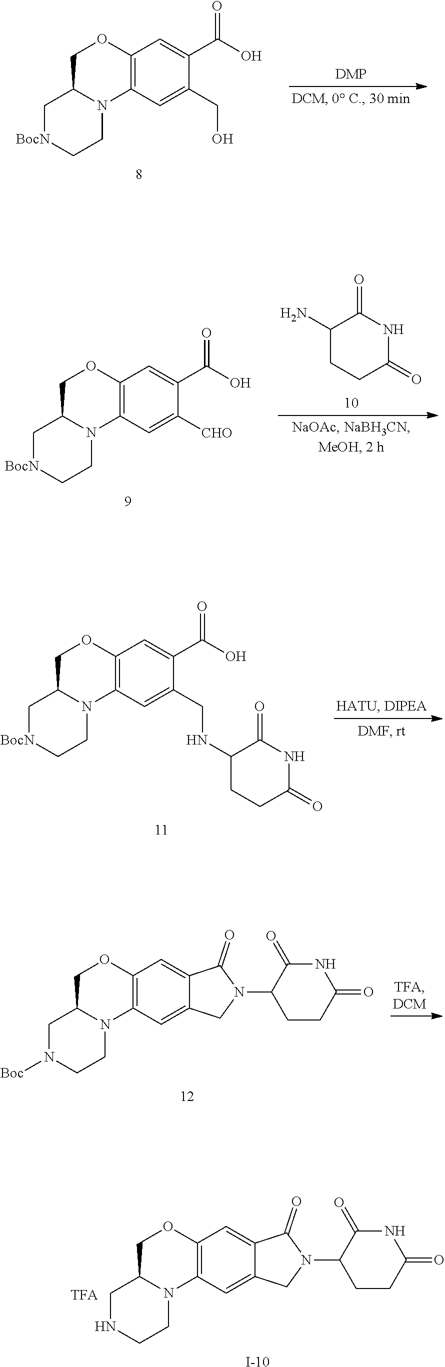

### Step 1:5-fluoro-6-nitroisobenzofuran-1 (3H)-one

To a solution of 5-fluoroisobenzofuran-1 (3H)-one (10 g, 65.8 mmol, 1.0 eq.) in H₂SO₄ (50 mL) was added KNO₃ (9.97 g, 98.7 mmol, 1.5 eq.) in portions. The reaction mixture was stirred at room temperature for 3 h and slowly poured into ice water. The organic phase was washed with brine, dried over Na₂SO₄ and filtered. The filtrate was evaporated in vacuo and the residue was purified by column chromatography on silica gel eluted with 0-50% EtOAc/hexane to afford 5-fluoro-6-nitroisobenzofuran-1 (3H)-one as white solid (10.4 g, 80% yield).

### Step 2, tert-butyl(S)-3-(hydroxymethyl)-4-(6-nitro-1-oxo-1,3-dihydroisobenzofuran-5-yl)piperazine-1-carboxylate

To a solution of 5-fluoro-6-nitroisobenzofuran-1 (3H)-one (1 g, 5.0 mmol, 1 eq.) and tert-butyl(S)-3-(hydroxymethyl)piperazine-1-carboxylate (1.7 g, 7.5 mmol, 1.5 eq.) in acetonitrile (10 mL) was added DIPEA (2.2 mL, 12.5 mmol, 2.5 eq.) and the mixture was stirred at 60° C. for 6 h. The mixture was concentrated and the residue was purified by column chromatography on silica gel eluted with 0-5% MeOH/DCM to afford tert-butyl(S)-3-(hydroxymethyl)-4-(6-nitro-1-oxo-1,3-dihydroisobenzofuran-5-yl)piperazine-1-carboxylate as yellow foam (1.3 g, 66% yield).

### Step 3: tert-butyl(S)-4-(6-amino-1-oxo-1,3-dihydroisobenzofuran-5-yl)-3-(hydroxymethyl)piperazine-1-carboxylate

To a solution of tert-butyl(S)-3-(hydroxymethyl)-4-(6-nitro-1-oxo-1,3-dihydroisobenzofuran-5-yl)piperazine-1-carboxylate (1.0 g, 2.8 mmol, 1 eq.) in McOH (15 mL) was added Pd/C(300 mg, 10% on carbon, wetted with ca, 55% water). The mixture was degassed and purged with H₂ three times and stirred at room temperature for 4 h. The catalyst was removed by filtration and the filtrate was evaporated to afford tert-butyl(S)-4-(6-amino-1-oxo-1,3-dihydroisobenzofuran-5-yl)-3-(hydroxymethyl)piperazine-1-carboxylate as light yellow foam (860 mg, 93% yield).

### Step 4: tert-butyl(S)-4-(6-bromo-1-oxo-1,3-dihydroisobenzofuran-5-yl)-3-(hydroxymethyl)piperazine-1-carboxylate

To a solution of tert-butyl tert-butyl(S)-4-(6-amino-1-oxo-1,3-dihydroisobenzofuran-5-yl)-3-(hydroxymethyl)piperazine-1-carboxylate (468 mg, 1.3 mmol, 1 eq.) in acetonitrile (25 mL) cooled in ice bath was added 1-BuONO (0.2 mL, 1.7 mmol, 1.3 eq.) and the mixture was stirred for 30 min. Then a solution of CuBr₂ (300 mg, 1.3 mmol, 1 eq.) in acetonitrile (6 mL) was added to the solution dropwise and the mixture was stirred at room temperature for 3 h. Then the mixture was diluted with EA (120 mL) and water (120 mL). The organic phase was washed with brine, dried over Na₂SO₄ and filtered. The filtrate was evaporated in vacuo and the residue was purified by column chromatography on silica gel eluted with 0-5% MeOH/DCM to afford tert-butyl(S)-4-(6-bromo-1-oxo-1,3-dihydroisobenzofuran-5-yl)-3-(hydroxymethyl)piperazine-1-carboxylate as brown oil (415 mg, 75% yield).

### Step 5: tert-butyl(S)-8-oxo-1,2,4a,5,8,10-hexahydroisobenzofuro[5,6-b]pyrazino[1,2-d][1,4]oxazine-3 (4H)-carboxylate

A mixture of tert-butyl(S)-4-(6-bromo-1-oxo-1,3-dihydroisobenzofuran-5-yl)-3-(hydroxymethyl)piperazine-1-carboxylate (140 mg, 0.3 mmol, 1 eq.), Pd(OAc)₂ (36.8 mg, 0.15 mmol, 0.5 eq.). JohnPhos (118 mg, 0.36 mmol, 1,2 eq.) and Cs₂CO₃ (214 mg, 0.7 mmol, 2 eq.) in toluene was degassed and purged with N₂ three times, and then the mixture was stirred at 90° C. for 3 h. The mixtures was cooled to room temperature, filtered through Celite, and the filtrate was concentrated. The residue was triturated with MeOH, and the solid was collected by filtration to afford tert-butyl(S)-8-oxo-1,2,4a,5,8,10-hexahydroisobenzofuro[5,6-b]pyrazino[1,2-d][1,4]oxazine-3 (4H)-carboxylate as yellow solid (90 mg, 80% yield).

### Step 6: (S)-3-(tert-butoxycarbonyl)-9-(hydroxymethyl)-1,2,3,4,4a,5-hexahydrobenzo[b]pyrazino[1,2-d][1,4]oxazine-8-carboxylic acid

To a solution of tert-butyl(S)-8-oxo-1,2,4a,5,8,10-hexahydroisobenzofuro[5,6-b]pyrazino[1,2-d][1,4]oxazine-3 (4H)-carboxylate (87 mg, 0.25 mmol, 1 eq.) in THF (3 mL) was added a solution of NaOH (60 mg, 1.3 mmol, 6 eq.) in H₂O (1 mL) and the mixture was stirred at 40° C. for 6 h. Then the mixture was concentrated and the residue was diluted with water (4 mL) and acidified to PH 3-4 with 2 N HCl. The mixture was extracted with DCM (10 mL) and the organic phase was washed with brine, dried over Na₂SO₄ and filtered. The filtrate was evaporated in vacuo to afford (S)-3-(tert-butoxycarbonyl)-9-(hydroxymethyl)-1,2,3,4,4a,5-hexahydrobenzo[b]pyrazino[1,2-d][1,4]oxazine-8-carboxylic acid as white powder (76 mg, 83% yield).

### Step 7: (S)-3-(tert-butoxycarbonyl)-9-formyl-1,2,3,4,4a,5-hexahydrobenzo[b]pyrazino[1,2-d][1,4]oxazine-8-carboxylic acid

To a solution of(S)-3-(tert-butoxycarbonyl)-9-(hydroxymethyl)-1,2,3,4,4a,5-hexahydrobenzo[b]pyrazino[1,2-d][1,4]oxazine-8-carboxylic acid (54 mg, 0.15 mmol, 1 eq.) in DCM (10 mL) cooled at 0° C. was added DMP (93.7 mg, 0.23 mmol, 1.5 eq.) in small portions and the mixture was stirred at 0° C. for 30 min. Then the mixture was diluted with DCM and washed with brine. The organic phase was dried over Na₂SO₄ and filtered. The filtrate was evaporated in vacuo to afford (S)-3-(tert-butoxycarbonyl)-9-formyl-1,2,3,4,4a,5-hexahydrobenzo[b]pyrazino[1,2-d][1,4]oxazine-8-carboxylic acid as yellow solid (50 mg, crude).

### Step 7: (4aS)-3-(tert-butoxycarbonyl)-9-(((2,6-dioxopiperidin-3-yl)amino)methyl)-1,2,3,4,4a,5-hexahydrobenzo[b]pyrazino[1,2-d][1,4]oxazine-8-carboxylic acid

To a mixture of(S)-3-(tert-butoxycarbonyl)-9-formyl-1,2,3,4,4a,5-hexahydrobenzo[b]pyrazino[1,2-d][1,4]oxazine-8-carboxylic acid (70 mg, 0.2 mmol, 1 eq.), 3-aminopiperidine-2,6-dione (47.6 mg, 0.3 mmol, 1.5 eq.) and NaOAc (23.7 mg, 0.3 mmol, 1.5 eq.) dissolved in MeOH (6 mL) was added NaBH₃CN(36 mg, 0.6 mmol, 3 eq.) and the mixture was stirred at room temperature for 1 h. Then the reaction was quenched with water and the mixture was purified by reverse phase column chromatography (0-50% Acetonitrile/0.05% formic acid) to afford (4aS)-3-(tert-butoxycarbonyl)-9-(((2,6-dioxopiperidin-3-yl)amino)methyl)-1,2,3,4,4a,5-hexahydrobenzo[b]pyrazino[1,2-d][1,4]oxazine-8-carboxylic acid as white powder (35 mg, 38% yield) after lyophilized.

### Step 8: tert-butyl (4aS)-9-(2,6-dioxopiperidin-3-yl)-8-oxo-1,2,4a,5,9,10-hexahydro-8H-pyrazino[1′,2′: 4,5][1,4]oxazino[2,3-f]isoindole-3 (4H)-carboxylate

To a solution of (4aS)-3-(tert-butoxycarbonyl)-9-(((2,6-dioxopiperidin-3-yl)amino)methyl)-1,2,3,4,4a,5-hexahydrobenzo[b]pyrazino[1,2-d][1,4]oxazine-8-carboxylic acid (47 mg, 0.1 mmol, 1 eq.) in DMF (2.5 mL) was added HATU (54 mg, 0.15 mmol, 1.5 eq.) followed by DIPEA (40 mg, 0.3 mmol, 3 eq.) and the mixture was stirred at room temperature for 1 h. Then the reaction was quenched with water and the mixture was purified by reverse phase column chromatography (0-50% acetonitrile/0.05% formic acid) to afford tert-butyl (4aS)-9-(2,6-dioxopiperidin-3-yl)-8-oxo-1,2,4a,5,9,10-hexahydro-8H-pyrazino[1′,2′: 4,5][1,4]oxazino[2,3-f]isoindole-3 (4H)-carboxylate (30 mg, 66% yield) as white powder.

### Step 9:3-((S)-8-oxo-1,2,3,4,4a,5,8,10-octahydro-9H-pyrazino[1′,2′: 4,5][1,4]oxazino[2,3-flisoindol-9-yl)piperidine-2,6-dione hydrochloride salt trifluoroacetate salt

A mixture of tert-butyl (4aS)-9-(2,6-dioxopiperidin-3-yl)-8-oxo-1,2,4a,5,9,10-hexahydro-8H-pyrazino[11,2′: 4,5][1,4]oxazino[2,3-f]isoindole-3 (4H)-carboxylate (30 mg, 1.0 eq) and HCl/dioxane (2 mL) was stirred at room temperature for 2 h. The mixture was concentrated to afford 3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione trifluoroacetate salt as white solid (26 mg, crude). LC-MS: [M+H]⁺=356.90, 1H NMR (400 MHZ, Methanol-d₄) δ 7.15 (s, 1H), 7.11 (d, J=5.7 Hz, 1H), 5.13-5.02 (m, 1H), 4.41-4,27 (m, 3H), 4.20 (d, J=13.6 Hz, 1H), 4.12-4.01 (m, 1H), 3.62-3.43 (m, 3H), 3.30-3.10 (m, 2H), 3.03-2.94 (m, 1H), 2.94-2.82 (m, 1H), 2.82-2,71 (m, 1H), 2.52-2.38 (m, 1H), 2.20-2.09 (m, 1H), 13C NMR (101 MHZ, MeOD) δ 174.68, 172.52, 172.49, 171.63, 171.59, 146.42, 146.37, 139.22, 139.20, 138.12, 138.08, 123.85, 123.78, 111.88, 108.70, 108.65, 66.94, 53.72, 53.57, 50.79, 50.75, 48.90, 48.68, 44.36, 44.17, 44.10, 43.63, 43.61, 32,35, 24.08.

### Intermediate 11: N-(2,6-dioxopiperidin-3-yl)-5-(piperazin-1-yl)picolinamide hydrochloride

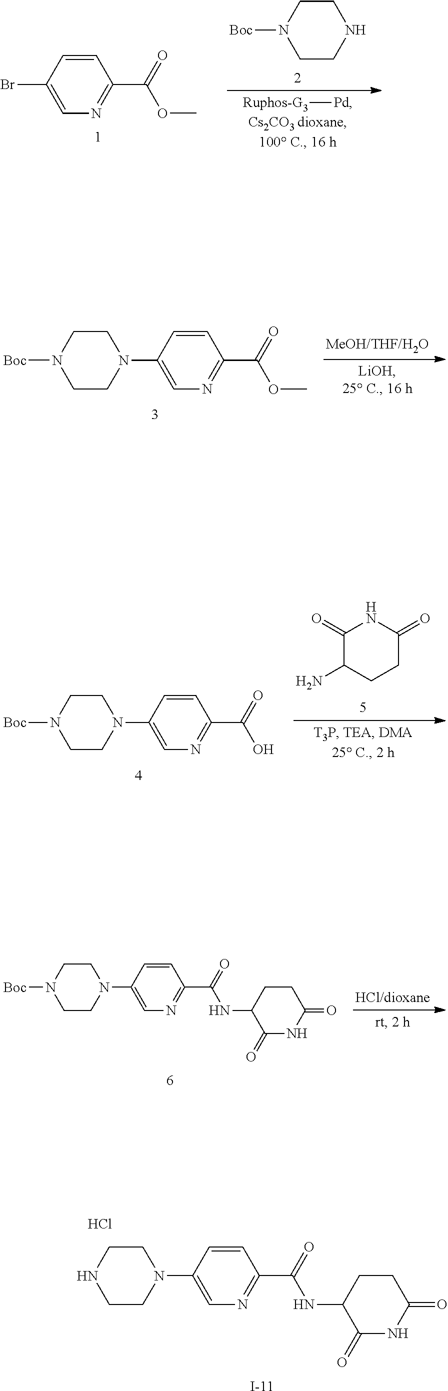

### Step 1: tert-butyl 4-(6-(methoxycarbonyl)pyridin-3-yl)piperazine-1-carboxylate

To a mixture of methyl 5-bromopicolinate (15 g, 69.4 mmol 1 eq.), tert-butyl piperazine-1-carboxylate (12.9 g, 69.4 mmol, 1 eq.) and Cs₂CO₃ (45 g, 139 mmol, 2 eq.) in dioxane (150 mL) was added Ruphos-G3-Pd(2.2 g, 3.5 mmol, 0.05 eq.) under Ar flow. The mixture was stirred at 100° C. for 16 h and cooled to room temperature. The residue was purified by column chromatography on silica gel eluted with 0-50% EtOAc/hexane to afford tert-butyl 4-(6-(methoxycarbonyl)pyridin-3-yl)piperazine-1-carboxylate (22 g, crude).

### Step 2:5-(4-(tert-butoxycarbonyl)piperazin-1-yl)picolinic acid

To a mixture of tert-butyl 4-(6-(methoxycarbonyl)pyridin-3-yl)piperazine-1-carboxylate (22 g, 68.5 mmol, 1 eq.) in MeOH (40 mL)/THF (100 mL)/H₂O (40 mL) was added LiOH (5.5 g, 137 mmol, 2 eq.) and the mixture was stirred at room temperature for 16 h. The mixture was concentrated and the residue was adjusted to pH 6 with 1N HCl. The precipitate was collected by filtration and dried in vacuo to afford 5-(4-(tert-butoxycarbonyl)piperazin-1-yl)picolinic acid (16.3 g, 76.4% yield).

### Step 3: tert-butyl 4-(6-((2,6-dioxopiperidin-3-yl) carbamoyl)pyridin-3-yl)piperazine-1-carboxylate

To a mixture of 5-(4-(tert-butoxycarbonyl)piperazin-1-yl)picolinic acid (1 g, 3.2 mmol, 1 eq.), 3-aminopiperidine-2,6-dione (537 mg, 3.2 mmol, 1 eq.) in DMA (5 ml) was added TEA (0.8 mL, 6.4 mmol, 2 eq.) and T₃P (3 mL, 4.8 mmol, 1.5 eq.). The reaction mixture was stirred at room temperature for 2 h, poured into water (50 mL) and extracted with EtOAc (20 mL). The organic phase was dried over Na₂SO₄ and filtered. The filtrate was evaporated in vacuo and the residue was purified by column chromatography on silica gel eluted with 0-100% EtOAc/hexane to afford tert-butyl 4-(6-((2,6-dioxopiperidin-3-yl) carbamoyl)pyridin-3-yl)piperazine-1-carboxylate (1.0 g, 78%) as white solid.

### Step 4: N-(2,6-dioxopiperidin-3-yl)-5-(piperazin-1-yl)picolinamide hydrochloride salt

A mixture of tert-butyl 4-(6-((2,6-dioxopiperidin-3-yl) carbamoyl)pyridin-3-yl)piperazine-1-carboxylate (1 g, 2.5 mmol,1.0 eq) in HCl/dioxane (5 mL) was stirred at room temperature for 2 h. The mixture was concentrated to afford N-(2,6-dioxopiperidin-3-yl)-5-(piperazin-1-yl)picolinamide hydrochloride salt as white solid (950 mg, crude).

### Intermediate 12: N-(2,6-dioxopiperidin-3-yl)-6-methoxy-5-(piperazin-1-yl)picolinamide hydrochloride

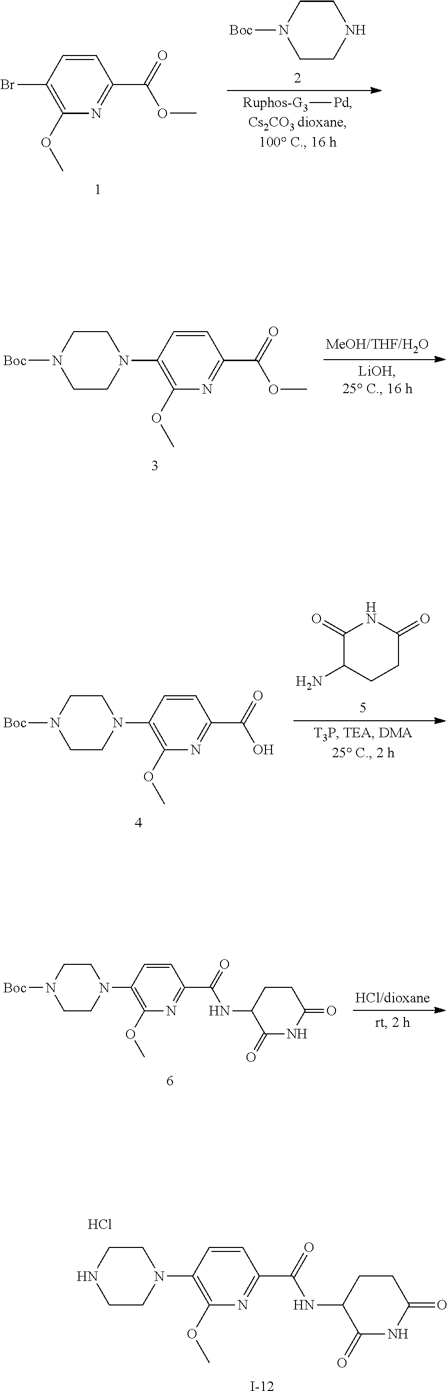

### Step 1: tert-butyl 4-(2-methoxy-6-(methoxycarbonyl)pyridin-3-yl)piperazine-1-carboxylate

To a mixture of methyl 5-bromo-6-methoxypicolinate (900 mg, 3.7 mmol, 1 eq.), tert-butyl piperazine-1-carboxylate (818 mg, 4,4 mmol, 1,2 eq.) and Cs₂CO₃ (1,4 g, 4,4 mmol, 1,2 eq.) in dioxane (15 mL) was added Ruphos-G3-Pd(153 mg, 0.18 mmol, 0.05 eq.) under Ar flow. The mixture was stirred at 100° C. for 16 h and cooled to room temperature. The residue was purified by column chromatography on silica gel eluted with 0-50% EtOAc/hexane to afford tert-butyl 4-(2-methoxy-6-(methoxycarbonyl)pyridin-3-yl)piperazine-1-carboxylate (770 mg, 50%).

### Step 2:5-(4-(tert-butoxycarbonyl)piperazin-1-yl)-6-methoxypicolinic acid

To a mixture of tert-butyl 4-(2-methoxy-6-(methoxycarbonyl)pyridin-3-yl)piperazine-1-carboxylate (70 mg, 0.2 mmol, 1 eq.) in MeOH (1 mL)/THF (1 mL)/H₂O (1 mL) was added LiOH (14 mg, 0.6 mmol, 3 eq.) and the mixture was stirred at room temperature for 16 h. The mixture was concentrated and the residue was adjusted to pH=6 with 1N HCl. The precipitate was collected by filtration and dried in vacuo to afford 5-(4-(tert-butoxycarbonyl)piperazin-1-yl)-6-methoxypicolinic acid (65 mg, crude).

### Step 3: tert-butyl 4-(6-((2,6-dioxopiperidin-3-yl) carbamoyl)-2-methoxypyridin-3-yl)piperazine-1-carboxylate

To a mixture of 5-(4-(tert-butoxycarbonyl)piperazin-1-yl)-6-methoxypicolinic acid (80 mg, 0.24 mmol, 1 eq.), 3-aminopiperidine-2,6-dione (46 mg, 0.28 mmol, 1,2 eq.) in DMA (3 ml) was added TEA (48 mg, 0.48 mmol, 2 eq.) and T₃P (152 mg, 0.48 mmol, 2 eq.). The reaction mixture was stirred at room temperature for 2 h, poured into water (30 mL) and extracted with EtOAc (10 mL). The organic phase was dried over Na₂SO₄ and filtered. The filtrate was evaporated in vacuo and the residue was purified by column chromatography on silica gel eluted with 0-100% EtOAc/hexane to afford tert-butyl 4-(6-((2,6-dioxopiperidin-3-yl) carbamoyl)-2-methoxypyridin-3-yl)piperazine-1-carboxylate (101 mg, 97%) as white solid.

### Step 4: N-(2,6-dioxopiperidin-3-yl)-6-methoxy-5-(piperazin-1-yl)picolinamide hydrochloride salt

A mixture of tert-butyl 4-(6-((2,6-dioxopiperidin-3-yl) carbamoyl)-2-methoxypyridin-3-yl)piperazine-1-carboxylate (450 mg, 1 mmol, 1.0 eq.) in HCl/dioxane (5 mL) was stirred at room temperature for 2 h. The mixture was concentrated to afford N-(2,6-dioxopiperidin-3-yl)-6-methoxy-5-(piperazin-1-yl)picolinamide hydrochloride salt as white solid (950 mg, crude).

### Intermediate 13: N-(2,6-dioxopiperidin-3-yl)-4-methoxy-5-(piperazin-1-yl)picolinamide hydrochloride salt

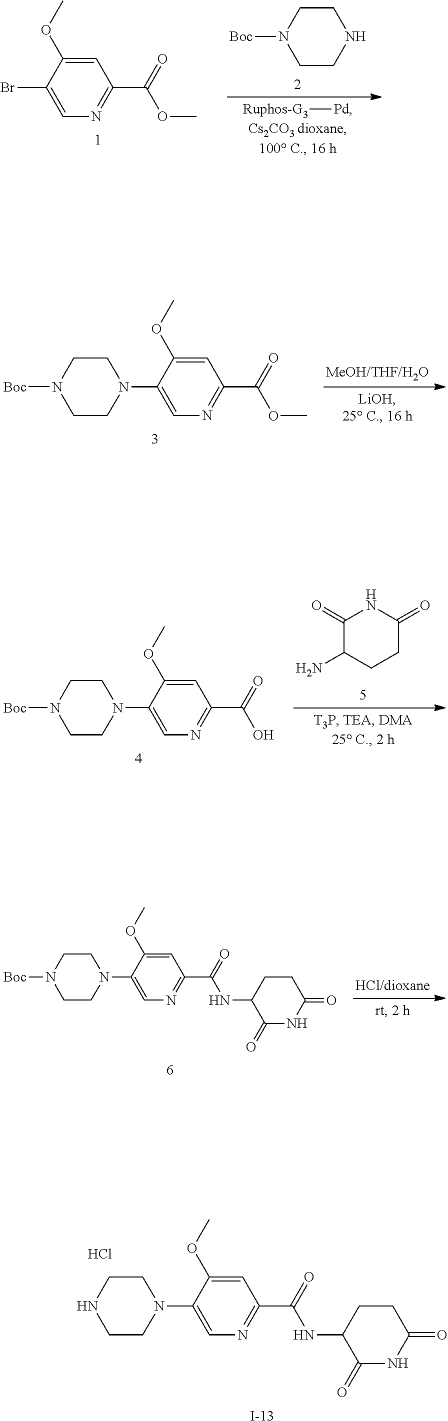

### Step 1: tert-butyl 4-(4-methoxy-6-(methoxycarbonyl)pyridin-3-yl)piperazine-1-carboxylate

To a mixture of methyl 5-bromo-4-methoxypicolinate (1 g, 4.0 mmol, 1 eq.), tert-butyl piperazine-1-carboxylate (818 mg, 4,4 mmol, 1,2 eq.) and Cs₂CO₃ (1,4 g, 4,4 mmol, 1,2 eq.) in dioxane (15 mL) was added Ruphos-G3-Pd(153 mg, 0.18 mmol, 0.05 eq.) under Ar flow. The mixture was stirred at 100° C. for 16 h and cooled to room temperature. The residue was purified by column chromatography on silica gel eluted with 0-50% EtOAc/hexane to afford tert-butyl 4-(4-methoxy-6-(methoxycarbonyl)pyridin-3-yl)piperazine-1-carboxylate (370 mg, 23% yield).

### Step 2:5-(4-(tert-butoxycarbonyl)piperazin-1-yl)-4-methoxypicolinic acid

To a mixture of tert-butyl 4-(2-methoxy-6-(methoxycarbonyl)pyridin-3-yl)piperazine-1-carboxylate (70 mg, 0.2 mmol, 1 eq.) in MeOH (1 mL)/THF (1 mL)/H₂O (1 mL) was added LiOH (14 mg, 0.6 mmol, 3 eq.) and the mixture was stirred at room temperature for 16 h. The mixture was concentrated and the residue was adjusted to pH=6 with 1N HCl. The precipitate was collected by filtration and dried in vacuo to afford 5-(4-(tert-butoxycarbonyl)piperazin-1-yl)-4-methoxypicolinic acid (65 mg, crude).

### Step 3: tert-butyl 4-(6-((2,6-dioxopiperidin-3-yl) carbamoyl)-4-methoxypyridin-3-yl)piperazine-1-carboxylate

To a mixture of 5-(4-(tert-butoxycarbonyl)piperazin-1-yl)-4-methoxypicolinic acid (160 mg, 0.48 mmol, 1 eq.), 3-aminopiperidine-2,6-dione (92 mg, 0.56 mmol, 1,2 eq.) in DMA (3 ml) was added TEA (97 mg, 0.97 mmol, 2 eq.) and T₃P (152 mg, 0.48 mmol, 1 eq.). The reaction mixture was stirred at room temperature for 2 h, poured into water (30 mL) and extracted with EtOAc (10 mL). The organic phase was dried over Na₂SO₄ and filtered. The filtrate was evaporated in vacuo and the residue was purified by column chromatography on silica gel eluted with 0-100% EtOAc/hexane to afford tert-butyl 4-(6-((2,6-dioxopiperidin-3-yl) carbamoyl)-4-methoxypyridin-3-yl)piperazine-1-carboxylate (97 mg, 95%) as white solid.

### Step 4: N-(2,6-dioxopiperidin-3-yl)-4-methoxy-5-(piperazin-1-yl)picolinamide hydrochloride salt

A mixture of tert-butyl 4-(6-((2,6-dioxopiperidin-3-yl) carbamoyl)-4-methoxypyridin-3-yl)piperazine-1-carboxylate (100 mg,0.22 mmol, 1.0 eq.) in HCl/dioxane (5 mL) was stirred at room temperature for 2 h. The mixture was concentrated to afford N-(2,6-dioxopiperidin-3-yl)-4-methoxy-5-(piperazin-1-yl)picolinamide hydrochloride salt as white solid (90 mg, crude).

### Intermediate 14. 3-(6-oxo-6,8-dihydro-2H,7H-spiro[furo[2,3-e]isoindole-3,4′-piperidin]-7-yl)piperidine-2,6-dione trifluoroacetate salt

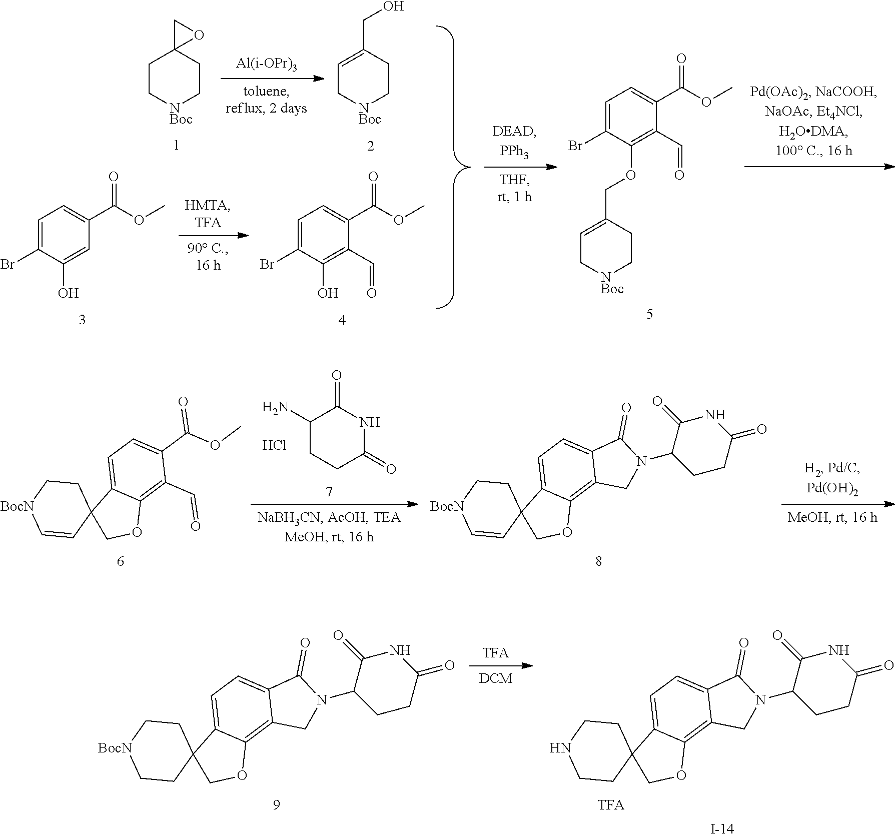

### Step 1: tert-butyl 4-(hydroxymethyl)-3,6-dihydropyridine-1 (2H)-carboxylate

A mixture of the tert-butyl 1-oxa-6-azaspiro[2.5]octane-6-carboxylate 1 (25 g, 117.4 mmol) and aluminium isopropoxide (35.9 g, 176 mmol) in anhydrous toluene (300 mL) was heated under reflux for 36 h. The reaction was allowed to cool and then poured into aqueous hydrogen chloride (1 M). The aqueous phase was extracted into EA and the organic extracts were dried (Na₂SO₄) then concentrated under reduced pressure. Chromatography of the residue gave the title compound 2 as a colorless oil (12 g, 48%)

### Step 2: methyl 4-bromo-2-formyl-3-hydroxybenzoate

To a solution of methyl 4-bromo-3-hydroxybenzoate 3 (18 g, 77.9 mmol) in TFA (150 mL) was added HMTA (41.5 g, 296 mmol). The solution was stirred at 90° C. overnight, 2N HCl was added, and a yellow solid formed. The mixture was stirred for 10 min and then additional 1 L water was added and stirred for 1 h. The mixture was filtered. The filter cake was dissolved in DCM and filtered on celite, dried, and then remove most of solvent in vacuo. The result mixture was triturated with MeOH and filtered to afford methyl 4-bromo-2-formyl-3-hydroxybenzoate 4 as a yellow solid (12 g, 59%).

### Step 3: tert-butyl 4-((6-bromo-2-formyl-3-(methoxycarbonyl)phenoxy)methyl)-3,6-dihydropyridine-1 (2H)-carboxylate

To a solution of compound 2 (6 g, 23.2 mmol, 1.0 eq.) in dry THF (50 ml), compound 4 (5.9 g, 27.8 mmol, 1,2 eq.) and PPh₃ (7.9 g, 30.1 mmol, 1.3 eq.) was added. The reaction mixture was cooled to 0° C. and DIAD (6.6 g, 32.4 mmol, 1,4 eq.) was added dropwise. The resultant mixture was then stirred 1h at room temperature. The solvent was evaporated at reduced pressure and the crude product was purified by silica gel column chromatography using 0-20% EtOAc/hexane. The desired product 5 was obtained as a yellow oil (4 g, 38%).

### Step 4: l′-(tert-butyl) 6-methyl 7-formyl-2′,3′-dihydro-1′H,2H-spiro[benzofuran-3,4′-pyridine]-1′,6-dicarboxylate

To a solution of compound 5 (4 g, 8.8 mmol, 1.0 eq.) in DMA (30 mL) was added NaCOOH (0.72 g, 10.6 mmol, 1,2 eq.), Et₄NCl·H₂O (1.95 g, 10.6 mmol, 1,2 eq), Pd(OAc)₂ (0.2 g, 0.88 mmol, 0.1 eq) and NaOAc (1,44 g, 17.6 mmol, 2 eq.). The mixture was purged with nitrogen and heated to 100° C. overnight. The mixture was diluted with ethyl acetate and washed with water. The organic layer was washed with brine and dried over sodium sulfate. The crude product was purified by silica gel column chromatography using 0-30% EtOAc/hexane to give compound 6 as a yellow oil (720 mg, yield 24%).

### Step 5: tert-butyl 7-(2,6-dioxopiperidin-3-yl)-6-oxo-2′,3′,7,8-tetrahydro-1′H,2H,6H-spiro[furo[2,3-e]isoindole-3,4′-pyridine]-1′-carboxylate

To a solution of compound 6 (780 mg, 2.09 mmol, 1 eq.) and compound 6 (344 mg, 2.09 mmol, 1 eq.) in MeOH (10 mL) was added TEA (211 mg, 2.09 mmol, 1 eq.) and AcOH (627 mg, 10.5 mmol, 5 eq.) followed by NaBH₃CN(395 mg, 6.27 mmol, 3 eq.). The mixture was stirred at room temperature for 16 h, diluted with EA, and washed with brine, then dried over sodium sulfate. The solvent was removed under reduced pressure. The crude product was purified by silica gel column chromatography using 0-100% EtOAc/hexane to give compound 8 as a white solid (400 mg, 42%).

### Step 6: lert-butyl 7-(2,6-dioxopiperidin-3-yl)-6-oxo-7,8-dihydro-2H,6H-spiro[furo[2,3-e]isoindole-3,4′-piperidine]-1′-carboxylate

To a solution of compound 8 (400 mg, 0.88 mmol, 1 eq.) in MeOH was added Pd/C(200 mg, 10% on Carbon, wetted with c.a,55% water) and Pd(OH)₂ (200 mg). The mixture was purged with H₂ and stirred at rt overnight under H₂. The mixture was filtered through Celite and the filtrate was concentrated. The crude product was purified by silica gel chromatography. The desired compound tcrt-butyl 7-(2,6-dioxopiperidin-3-yl)-6-oxo-7,8-dihydro-2H,6H-spiro[furo[2,3-e]isoindole-3,4′-piperidine]-1′-carboxylate was obtained as white solid (220 mg, 55%). LC/MS (ESI) m/z: 356.2 [M+H]⁺, ¹H NMR (400 MHZ, DMSO) δ 10.97 (s, 1H), 7.42 (d, J=7.2 Hz, 1H), 7.26 (d, J=7.6 Hz, 1H), 5.11-5.06 (m, 1H), 4.62-4.57 (m, 2H), 4.38 (d, J=17.2 Hz, 1H), 4.21 (d, J=17.2 Hz, 1H), 3.95-3.92 (m, 2H), 2.95-2.83 (m, 3H), 2.61-2.56 (m, 1H), 2.47-2.39 (m, 1H), 1.98-1.96 (m, 1H), 1.83-1.77 (m, 2H), 1.71-1.65 (m, 2H), 1.42 (s, 9H),

### Step 7:3-(6-oxo-6,8-dihydro-2H,7H-spiro[furo[2,3-e]isoindole-3,4′-piperidin]-7-yl)piperidine-2,6-dione trifluoroacetate salt

Compound 9 was treated with TFA in DCM at room temperature to de-protect the N-Boc group to provide intermediate I-14. LC/MS(ESI) m/z: 356.15. 1H NMR (400 MHZ, CDCl₃) δ 8.00 (s, 1H), 7.50 (d, J=7.7 Hz, 1H), 7.28 (s, 1H), 5.23 (dd, J=13.3, 5.1 Hz, 1H), 4.55 (d, J=1.4 Hz, 2H), 4.46 (d, J=16.0 Hz, 1H), 4.32 (d, J=16.0 Hz, 1H), 4.15 (s, 2H), 3.01-2,77 (m, 4H), 2.38 (dd, J=13.1, 5.0 Hz, 1H), 2.29-2.17 (m, 1H), 1.92 (t, J=12.5 Hz, 2H), 1.83-1.72 (m, 2H),

### Intermediate 15:2-(dimethoxymethyl)-7-azaspiro[3.5]nonane

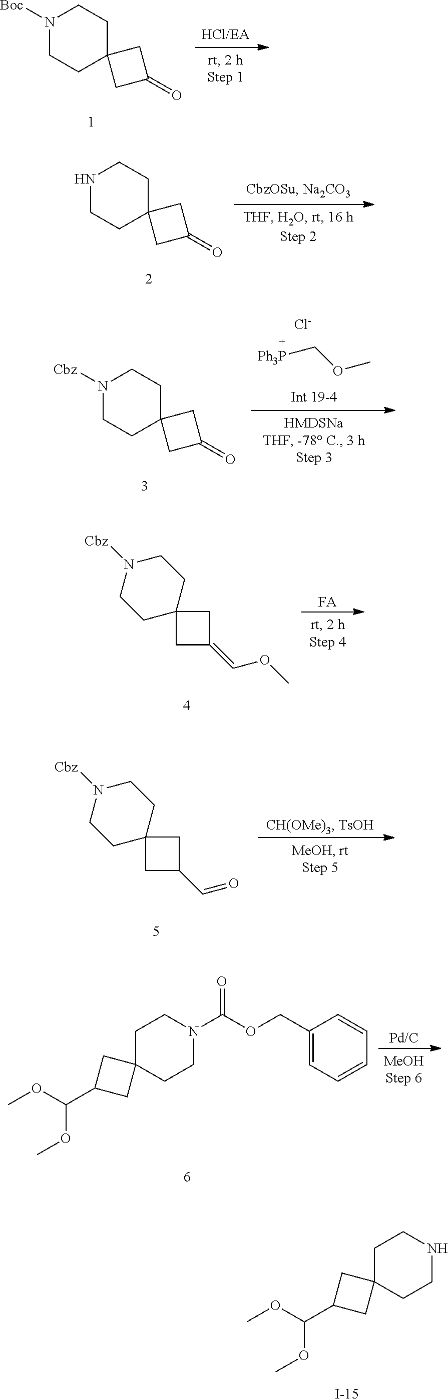

### Step 1: benzyl 2-oxo-7-azaspiro[3.5]nonane-7-carboxylate

To a stirred solution of tert-butyl 2-oxo-7-azaspiro[3.5]nonane-7-carboxylate (24 g, 0.1 mol, 1 eq.) in EA (50 mL) at room temperature was added conc. HCl (45 mL, 0.5 mol, 5 eq.) slowly and the reaction mixture was stirred at rt for 1 hour. Once the reaction was completed, the mixture was diluted with EA (150 mL), poured into Na₂CO₃ suspension (106 g, 1 mol, 10 eq., in 500 mL of water) and the mixture was stirred for 20 min. To the mixture was added CbzOSu (25 g, 0.1 mmol, 1 eq.) and the mixture was stirred for 1 h. The organic phase was separated, washed with brine, dried, concentrated and the residue was purified by silica column chromatography eluting with 50% EA in PE to give compound benzyl 2-oxo-7-azaspiro[3.5]nonane-7-carboxylate (27 g, 0.1 mol, 100%) as a light yellow oil.

### Step 2: benzyl 2-(methoxymethylene)-7-azaspiro[3.5]nonane-7-carboxylate

To a stirred solution of (methoxymethyl)triphenylphosphonium chloride (68 g, 0.2 mol, 2 eq) in dried THF (300 mL) cooled at −70° C. was added NaHMDS(100 mL, 0.2 mol, 2 eq.) dropwise and the mixture was warmed to 0° C. slowly and stirred for 2 h. Then the mixture was cooled at −70° C. and a solution of benzyl 2-oxo-7-azaspiro[3.5]nonane-7-carboxylate (27 g, 0.1 mol, 1 eq.) in THF (50 mL) was added. The mixture was warmed to rt slowly and stirred for 2 h. TLC was done to detect the process of the reaction. Once no starting material was left, the mixture was quenched by NH₄Cl solution (500 mL) and diluted with EA (200 mL). The organic phase was separated, washed with brine, dried, concentrated and the residue was purified by silica column chromatography eluting with 30% EA in PE to give compound benzyl 2-(methoxymethylene)-7-azaspiro[3.5]nonane-7-carboxylate (20 g, 0.067 mol, 67%) as a light yellow oil. Step 3: benzyl 2-(dimethoxymethyl)-7-azaspiro[3.5]nonane-7-carboxylate

A solution of benzyl 2-(methoxymethylene)-7-azaspiro[3.5]nonane-7-carboxylate (24 g. 0.67 mol, 1 eq.) in FA (50 mL) was stirred at rt for 4 hours. TLC were done to detect the process of the reaction. Once the reaction was completed, the mixture concentrated and the residue was dissolved in MeOH (120 mL). To the mixture was added CH(OMe)₃ (10.6 g, 0.1 mol, 1.5 eq.) followed by TsOH. H₂O (1.5 g, 0.07 mol, 0.1 eq.) and the mixture was stirred at 70° C. for 12 h. TLC were done to detect the process of the reaction. Once the reaction was completed, the mixture was concentrated and the residue was purified by silica column chromatography eluting with 20% EA in PE to give compound benzyl 2-(dimethoxymethyl)-7-azaspiro[3.5]nonane-7-carboxylate (14.6 g, 0.44 mol, 67%) as light yellow oil.

### Step 4:2-(dimethoxymethyl)-7-azaspiro[3.5]nonane

To a solution of benzyl 2-(dimethoxymethyl)-7-azaspiro[3.5]nonane-7-carboxylate (14.6 g, 0.44 mol, 1 eq.) in MeOH (100 mL) was added Pd/C(4 g, 10% on Carbon, wetted with ca, 55% water) and the mixture was stirred at rt for 12 hours under H₂ (balloon). TLC were done to detect the process of the reaction. Once the reaction was completed, the catalyst was removed by filtration and the filtrate was concentrated to give compound 2-(dimethoxymethyl)-7-azaspiro[3.5]nonane (8.9 g, 0.44 mol, 100%) as a white paste.

LCMS [M+H]: 200.0. 1H NMR (400 MHZ, DMSO-d₆) δ 4.57 (d, J=6.8 Hz, 1H), 3.20 (m, 6H), 2.61 (s, 2H), 2.47-2.43 (m, 1H), 1.74 (t, 2H), 1.54-1.44 (m, 4H), 1.34 (t, 2H),

### Intermediate 16:4-(dimethoxymethyl)piperidine

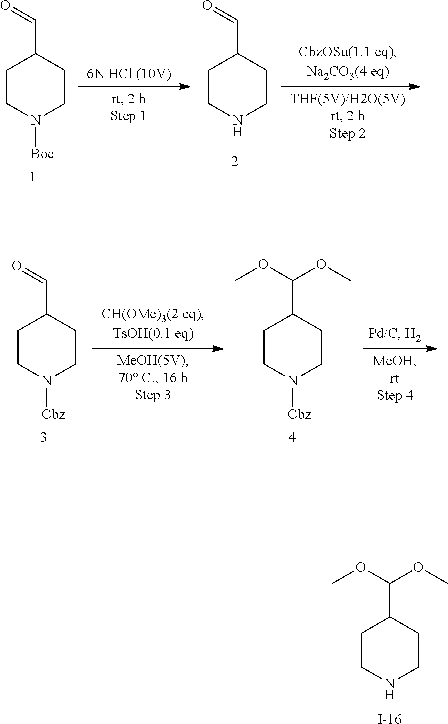

### Step 1 and step 2: benzyl 4-formylpiperidine-1-carboxylate

To a stirred solution of compound tert-butyl 4-formylpiperidine-1-carboxylate (500 g, 2.2 mol, 1 eq.) in EA (500 mL) at room temperature was added conc. HCl (600 mL, 6.6 mol, 3 eq.) slowly and the reaction mixture was stirred at rt for 1 hour. Once the reaction was completed, the mixture was diluted with EA (500 mL), poured into Na₂CO₃ suspension (1160 g, 11 mol, 5 eq., in 3000 ml of water) and the mixture was stirred for 20 min. To the mixture was added CbzOSu (550 g, 2.2 mmol, 1 eq.) and the mixture was stirred for 1 h. The organic phase was separated, washed with brine, dried, concentrated and the residue was purified by silica column chromatography eluting with 50% EA in PE to give compound benzyl 4-formylpiperidine-1-carboxylate (550 g, 2.1 mol, 95%) as a light yellow oil.

### Step 3: benzyl 4-(dimethoxymethyl)piperidine-1-carboxylate

To a solution of benzyl 4-formylpiperidine-1-carboxylate (150 g, 0.5 mol, 1 eq.) in MeOH (500 mL) was added CH(OMe)₃ (212 g, 1 mol, 2 eq.) followed by TsOH. H₂O (19 g, 0.1 mol, 0.1 eq.) and the mixture was stirred at 70° C. for 12 h. Once the reaction was completed, the mixture was concentrated and the residue was purified by silica column chromatography eluting with 20% EA in PE to give compound benzyl 4-(dimethoxymethyl)piperidine-1-carboxylate (120 g, 0.41 mol, 82%) as light yellow oil.

### Step 4:4-(dimethoxymethyl)piperidine

To a solution of compound benzyl 4-(dimethoxymethyl)piperidine-1-carboxylate (120 g. 0.44 mol, 1 eq.) in MeOH (400 mL) was added Pd/C(20 g, 10% on Carbon, wetted with ca, 55% water) and the mixture was stirred at rt for 12 hours under H₂ (balloon). Once the reaction was completed, the catalyst was removed by filtration and the filtrate was concentrated to give compound 4-(dimethoxymethyl)piperidine (65 g, 0.41 mol, 100%) as a white paste.

### Intermediate 17:7-(dimethoxymethyl)-2-azaspiro[3.5]nonane

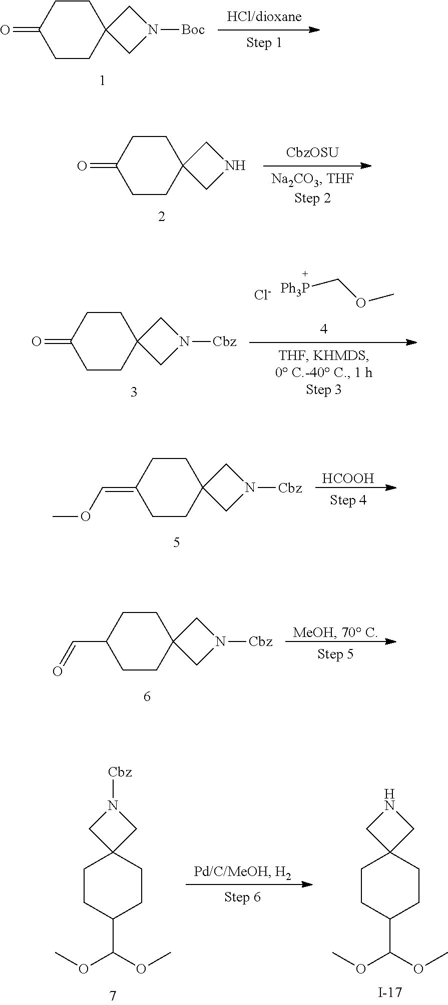

### Step 1: benzyl 7-oxo-2-azaspiro[3.5]nonane-2-carboxylate

To a stirred solution of compound tert-butyl 7-oxo-2-azaspiro[3.5]nonane-2-carboxylate (2.4 g, 10 mmol, 1 eq.) in EA (5 mL) at room temperature was added conc. HCl (4.5 mL, 50 mol, 5 eq.) slowly and the reaction mixture was stirred at rt for 1 hour. Once the reaction was completed, the mixture was diluted with EA (15 mL), poured into Na₂CO₃ suspension (10.6 g, 0.1 mol, 10 eq., in 50 mL of water) and the mixture was stirred for 20 min. To the mixture was added CbzOSu (2.5 g, 10 mmol, 1 eq.) and the mixture was stirred for 1 h. The organic phase was separated, washed with brine, dried, concentrated and the residue was purified by silica column chromatography eluting with 50% EA in PE to give compound benzyl 7-oxo-2-azaspiro[3.5]nonane-2-carboxylate (2.7 g, 0.1 mol, 100%) as a light yellow oil. Step 2: benzyl 7-(methoxymethylene)-2-azaspiro[3.5]nonane-2-carboxylate

To a stirred solution of (methoxymethyl)triphenylphosphonium chloride (6.8 g, 20 mol, 2 eq) in dried THF (30 mL) cooled at −70° C. was added NaHMDS(10 mL, 20 mol, 2 eq.) dropwise and the mixture was warmed to 0° C. slowly and stirred for 2 h. Then the mixture was cooled at-70° C. and a solution of benzyl 7-oxo-2-azaspiro[3.5]nonane-2-carboxylate (2.7 g, 0.1 mol, 1eq.) in THF (5 mL) was added. The mixture was warmed to rt slowly and stirred for 2 h. TLC was done to detect the process of the reaction. Once no starting material was left, the mixture was quenched by NH₄Cl solution (50 mL) and diluted with EA (20 mL). The organic phase was separated, washed with brine, dried, concentrated and the residue was purified by silica column chromatography eluting with 30% EA in PE to give compound benzyl 7-(methoxymethylene)-2-azaspiro[3.5]nonane-2-carboxylate (2.2 g, 7.3 mmol, 73%) as a light yellow oil.

### Step 3: benzyl 7-(dimethoxymethyl)-2-azaspiro[3.5]nonane-2-carboxylate

A solution of 7-(methoxymethylene)-2-azaspiro[3.5]nonane-2-carboxylate (2.2 g, 7.3 mol, 1 eq.) in FA (5 mL) was stirred at rt for 4 hours. TLC were done to detect the process of the reaction. Once the reaction was completed, the mixture concentrated and the residue was dissolved in MeOH (12 mL). To the mixture was added CH(OMe)₃ (1.06 g, 10 mol, 1.5 eq.) followed by TsOH. H₂O (190 mg, 1 mol, 0.1 eq.) and the mixture was stirred at 70° C. for 12 h. TLC were done to detect the process of the reaction. Once the reaction was completed, the mixture was concentrated and the residue was purified by silica column chromatography eluting with 20% EA in PE to give compound benzyl 7-(dimethoxymethyl)-2-azaspiro[3.5]nonane-2-carboxylate (1.5 g. 4,4 mmol, 67%) as light yellow oil.

### Step 4:7-(dimethoxymethyl)-2-azaspiro[3.5]nonane

To a solution of compound benzyl 7-(dimethoxymethyl)-2-azaspiro[3.5]nonane-2-carboxylate (1.5 g, 4,4 mol, 1 eq.) in MeOH (10 mL) was added Pd/C(400 mg, 10% on Carbon, wetted with ca, 55% water) and the mixture was stirred at rt for 12 hours under H₂ (balloon). TLC were done to detect the process of the reaction. Once the reaction was completed, the catalyst was removed by filtration and the filtrate was concentrated to give compound 7-(dimethoxymethyl)-2-azaspiro[3.5]nonane (810 mg, 4 mol, 90%) as a white paste. LCMS: 200 [M+H]⁺.

### Intermediate 18. 2-(dimethoxymethyl)-7-azaspiro[3.5]nonane

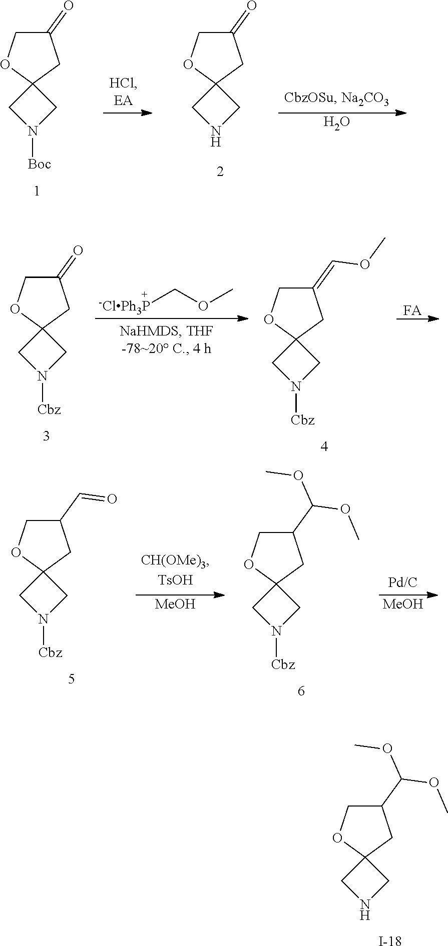

### Step 1: benzyl 7-oxo-5-oxa-2-azaspiro[3,4]octane-2-carboxylate

To a stirred solution of tert-butyl 7-oxo-5-oxa-2-azaspiro[3,4]octane-2-carboxylate (10 g, 40 mmol, 1 eq.) in EA (50 mL) at room temperature was added conc. HCl (20 mL, 0.2 mol, 5 eq.) slowly and the reaction mixture was stirred at room temperature for 1 h. Then the mixture was diluted with EA (150 mL), poured into Na₂CO₃ suspension (40 g, 0.4 mol, 10 eq, in 500 mL of water) and the mixture was stirred for 20 min. To the mixture was added CbzOSu (10 g, 40 mmol, 1 eq.) and the mixture was stirred for 1 h. The organic phase was separated, washed with brine, dried, concentrated and the residue was purified by column chromatography on silica gel eluted with 0-40% EtOAc/hexane to afford benzyl 7-oxo-5-oxa-2-azaspiro[3,4]octane-2-carboxylate (10.8 g, 100% yield) as light yellow oil.

### Step 2: benzyl (E)-7-(methoxymethylene)-5-oxa-2-azaspiro[3,4]octane-2-carboxylate

To a stirred solution of (methoxymethyl)triphenylphosphonium chloride (28.3 g, 80 mmol, 2 eq.) in dried THF (300 mL) cooled at −70° C. was added NaHMDS(40 mL, 160 mmol, 2 eq.) dropwise and the mixture was warmed to 0° C. slowly and stirred for 2 h. Then the mixture was cooled at −70° C. and a solution of benzyl 2-oxo-7-azaspiro[3.5]nonane-7-carboxylate (10.8 g, 40 mmol, 1 eq.) in THF (20 mL) was added. The mixture was warmed to room temperature slowly and stirred for 2 h. The mixture was quenched by NH₄Cl solution (200 mL) and diluted with EA (100 mL). The organic phase was separated, washed with brine, dried, concentrated and the residue was purified by column chromatography on silica gel eluted with 0-40% EtOAc/hexane to afford benzyl (E)-7-(methoxymethylene)-5-oxa-2-azaspiro[3,4]octane-2-carboxylate (4.5 g, 16 mmol, 40% yield) as light yellow oil.

### Step 3: benzyl 7-(dimethoxymethyl)-5-oxa-2-azaspiro[3,4]octane-2-carboxylate

A solution of benzyl (E)-7-(methoxymethylene)-5-oxa-2-azaspiro[3,4]octane-2-carboxylate (4.5 g, 16 mmol, 1 eq.) in FA (20 mL) was stirred at room temperature for 4 h. The mixture was concentrated, and the residue was dissolved in MeOH (20 mL). To the mixture was added CH(OMe)₃ (2.5 g, 24 mol, 1.5 eq.) followed by TsOH-H₂O (3.1 g, 1.6 mmol, 0.1 eq.) and the mixture was stirred at 70° C. for 12 h. The mixture was concentrated and the residue was purified by column chromatography on silica gel eluted with 0-40% EtOAc/hexane to afford benzyl 7-(dimethoxymethyl)-5-oxa-2-azaspiro[3,4]octane-2-carboxylate (2.5 g, 49% yield) as light yellow oil.

### Step 4:2-(dimethoxymethyl)-7-azaspiro[3.5]nonane

To a solution of benzyl 7-(dimethoxymethyl)-5-oxa-2-azaspiro[3,4]octane-2-carboxylate (2.5 g, 7.8 mmol, 1 eq.) in MeOH (20 mL) was added Pd/C(1 g, 10% on carbon, wetted with ca. 55% water) and the mixture was stirred at room temperature for 12 h under H₂ (balloon). The catalyst was removed by filtration and the filtrate was concentrated to afford 2-(dimethoxymethyl)-7-azaspiro[3.5]nonane (1.5 g, crude) as white paste.

### Intermediate 19. 3-(dimethoxymethyl)-1-oxa-8-azaspiro[4.5]decane

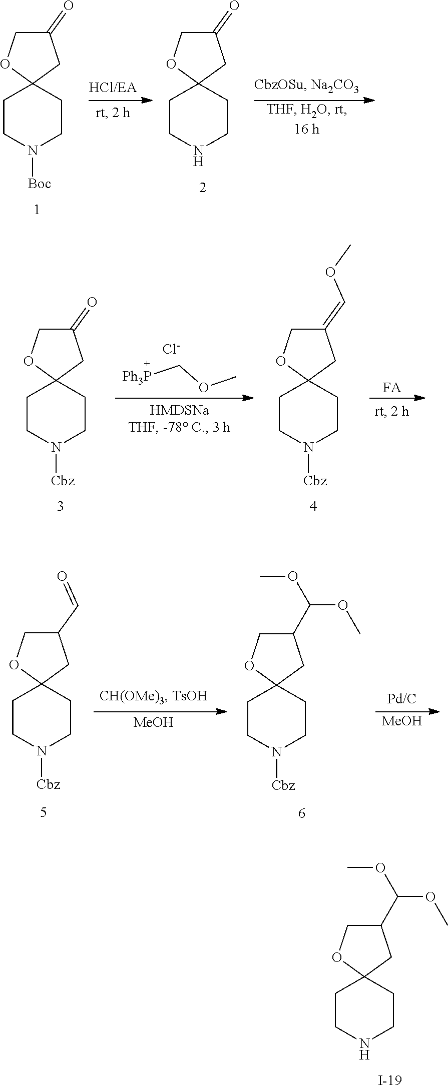

### Step 1: benzyl 3-oxo-1-oxa-8-azaspiro[4.5]decane-8-carboxylate

To a stirred solution of tert-butyl 3-oxo-1-oxa-8-azaspiro[4.5]decane-8-carboxylate (11 g, 40 mmol, 1 eq.) in EA (50 mL) at room temperature was added conc. HCl (20 mL, 0.2 mol, 5 eq.) slowly and the reaction mixture was stirred at room temperature for 1 h. Then the mixture was diluted with EA (150 mL), poured into Na₂CO₃ suspension (40 g, 0.4 mol, 10 eq, in 500 mL of water) and the mixture was stirred for 20 min. To the mixture was added CbzOSu (10 g, 40 mmol, 1 eq.) and the mixture was stirred for 1 h. The organic phase was separated, washed with brine, dried, concentrated and the residue was purified by column chromatography on silica gel eluted with 0-40% EtOAc/hexane to afford benzyl 7-oxo-5-oxa-2-azaspiro[3,4]octane-2-carboxylate (11 g, 95% yield) as light yellow oil.

### Step 2: benzyl (Z)-3-(methoxymethylene)-1-oxa-8-azaspiro[4.5]decane-8-carboxylate

To a stirred solution of (methoxymethyl)triphenylphosphonium chloride (28.3 g, 80 mmol, 2 eq.) in dried THF (300 mL) cooled at −70° C. was added NaHMDS(40 mL, 160 mmol, 2 eq.) dropwise and the mixture was warmed to 0° C. slowly and stirred for 2 h. Then the mixture was cooled at −70° C. and a solution of benzyl 3-oxo-1-oxa-8-azaspiro[4.5]decane-8-carboxylate (11 g, 40 mmol, 1 eq.) in THF (20 mL) was added. The mixture was warmed to room temperature slowly and stirred for 2 h. The mixture was quenched by NH₄Cl solution (200 mL) and diluted with EA (100 mL). The organic phase was separated, washed with brine, dried, concentrated and the residue was purified by column chromatography on silica gel eluted with 0-40% EtOAc/hexane to afford benzyl (Z)-3-(methoxymethylene)-1-oxa-8-azaspiro[4.5]decane-8-carboxylate (5.4 g, 17 mmol, 44% yield) as light yellow oil.

### Step 3: benzyl 3-(dimethoxymethyl)-1-oxa-8-azaspiro[4.5]decane-8-carboxylate

A solution of benzyl (E)-7-(methoxymethylene)-5-oxa-2-azaspiro[3,4]octane-2-carboxylate (5.4 g, 17 mmol, 1 eq.) in FA (20 mL) was stirred at room temperature for 4 h. The mixture was concentrated, and the residue was dissolved in MeOH (20 mL). To the mixture was added CH(OMe)₃ (2.5 g, 24 mol, 1.5 eq.) followed by TsOH.H₂O (3.1 g, 1.6 mmol, 0.1 eq.) and the mixture was stirred at 70° C. for 12 h. The mixture was concentrated and the residue was purified by column chromatography on silica gel eluted with 0-40% EtOAc/hexane to afford benzyl 3-(dimethoxymethyl)-1-oxa-8-azaspiro[4.5]decane-8-carboxylate (3.2 g, 50% yield) as light yellow oil.

### Step 4:3-(dimethoxymethyl)-1-oxa-8-azaspiro[4.5]decane

To a solution of benzyl 7-(dimethoxymethyl)-5-oxa-2-azaspiro[3,4]octane-2-carboxylate (3.5 g, 10 mmol, 1 eq.) in McOH (30 mL) was added Pd/C(1 g, 10% on Carbon, wetted with ca. 55% water) and the mixture was stirred at room temperature for 12 h under H₂ (balloon). The catalyst was removed by filtration and the filtrate was concentrated to afford 3-(dimethoxymethyl)-1-oxa-8-azaspiro[4.5]decane (2.1 g, crude) as white paste.

### Intermediate 20 5-(4-(2-(dimethoxymethyl)-7-azaspiro[3.5]nonan-7-yl)-5-fluoro-2-methoxyphenyl)-6-phenyl-5,6,7,8-tetrahydronaphthalen-2-ol

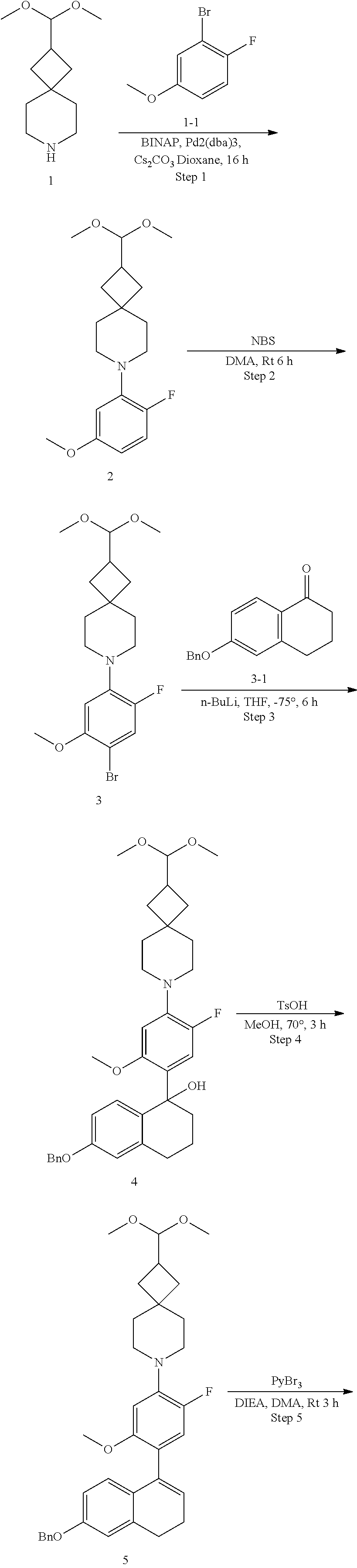

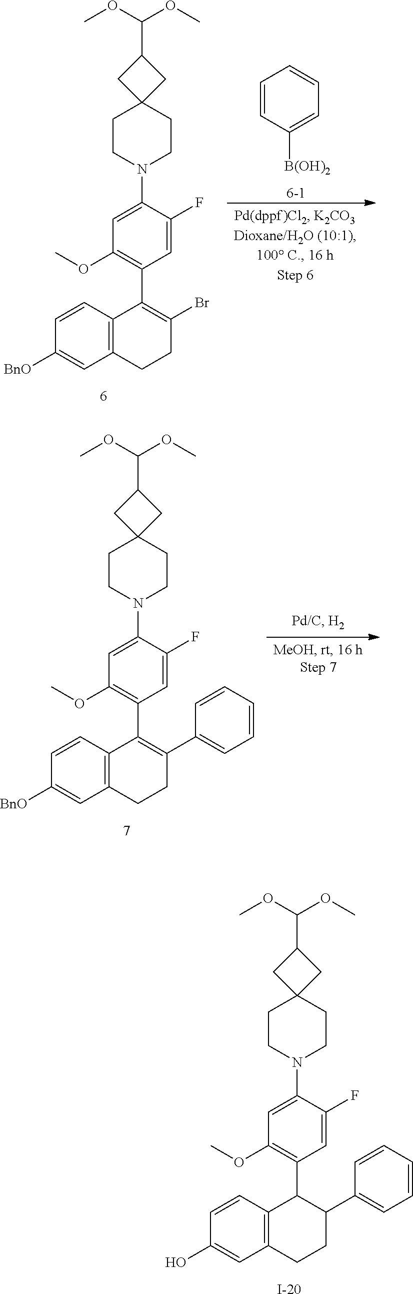

### Step 1:2-(dimethoxymethyl)-7-(2-fluoro-5-meth2-(dimethoxymethyl)-7-(2-fluoro-5-methoxyphenyl)-7-azaspiro[3.5]nonaneoxyphenyl)-7-azaspiro[3.5]nonane

To a mixture of 2-(dimethoxymethyl)-7-azaspiro[3.5]nonane (10.0 g, 50.18 mmol, 1 eq.), 2-bromo-1-fluoro-4-methoxybenzene (11.0 g, 55.19 mmol, 0.1 eq.) and Cs₂CO₃ (32.7 g, 100.35 mmol, 2 eq.) in dioxane (60 ml) were added BINAP (3.12 g, 5.02 mmol, 0.1 eq.) and Pd₂(dba) 3 (4.59 g, 5.02 mmol, 0.1 eq.). The whole mixture was stirred at 100° C. for 16 hours under Ar. Once finished, the mixture was cooled to room temperature followed by dilution with ethyl acetate (150 mL). The mixture was washed with water (100 mL). The organic layers were collected, washed with brine (2×100 mL), dried over Na₂SO₄, and filtered. The filtrate was evaporated in vacuo. The residue was purified by column chromatography on silica gel eluted with 0-40% EtOAc/hexane to afford 2-(dimethoxymethyl)-7-(2-fluoro-5-methoxyphenyl)-7-azaspiro[3.5]nonane (10.20 g, 62.86% yield) as yellow oil.

LC-MS purity: 100% (UV at 254 nm), 324.5 [M+H]⁺.

### Step 2:7-(4-bromo-2-fluoro-5-methoxyphenyl)-2-(dimethoxymethyl)-7-azaspiro[3.5]nonane

To a mixture of 2-(dimethoxymethyl)-7-(2-fluoro-5-methoxyphenyl)-7-azaspiro[3.5]nonane (10.20 g, 31.54 mmol, 1.0 eq.) in DMA (50 ml) was added N-bromosuccinimide (6.74 g, 37.85 mmol, 1,2 eq.) at 10° C. The whole mixture was then stirred at room temperature for 6 hours. Once finished, the mixture was diluted with ethyl acetate (150 mL) and washed with water (200 mL). The organic layer was collected, washed with brine (2×100 mL), dried over Na₂SO₄, and filtered. The filtrate was evaporated in vacuo. The residue was purified by column chromatography on silica gel eluted with 0-45% EtOAc/hexane to afford 7-(4-bromo-2-fluoro-5-methoxyphenyl)-2-(dimethoxymethyl)-7-azaspiro[3.5]nonane (10.00 g, 78.81% yield) as yellow solid.

LC-MS purity: 97.2% (UV at 254 nm), 402.2 [M+H]⁺.

### Step 3:6-(benzyloxy)-1-(4-(2-(dimethoxymethyl)-7-azaspiro[3.5]nonan-7-yl)-5-fluoro-2-methoxyphenyl)-1,2,3,4-tetrahydronaphthalen-1-ol

To a mixture of 7-(4-bromo-2-fluoro-5-methoxyphenyl)-2-(dimethoxymethyl)-7-azaspiro[3.5]nonane (2.70 g, 6.71 mmol, 1 eq) in dry THF (10 mL) under Argon was added dropwise n-BuLi (2.50 M, 3.22 mL, 1,2 eq). The mixture was stirred at −78° C. for 1.5 h followed by the dropwise addition of 6-(benzyloxy)-3,4-dihydronaphthalen-1 (2H)-one (1.86 g, 7.38 mmol, 1,1 eq) in dry THF (10 mL). The mixture was then stirred at −78° C. for 3 h. Once TLC (PE:EA=5:1) showed the starting material was consumed completely, the mixture was quenched by the addition of the saturated aqueous NH₄Cl at 0° C. The mixture was poured into H₂O (40 mL), extracted with EtOAc (30.0 mL×2), and the combined organic layer was washed with brine, dried over Na₂SO₄, filtered, and concentrated in vacuum. The crude product was purified by silica gel column chromatography using 0-40% EtOAc/hexane to afford 6-(benzyloxy)-1-(4-(2-(dimethoxymethyl)-7-azaspiro[3.5]nonan-7-yl)-5-fluoro-2-methoxyphenyl)-1,2,3,4-tetrahydronaphthalen-1-ol (1.30 g, 33.65% yield) as a white solid.

LC-MS purity: 96.2% (UV at 254 nm), 575.3 [M+H]⁺.

### Step 4:7-(4-(6-(benzyloxy)-3,4-dihydronaphthalen-1-yl)-2-fluoro-5-methoxyphenyl)-2-(dimethoxymethyl)-7-azaspiro[3.5]nonane

To a mixture of 6-(benzyloxy)-1-(4-(2-(dimethoxymethyl)-7-azaspiro[3.5]nonan-7-yl)-5-fluoro-2-methoxyphenyl)-1,2,3,4-tetrahydronaphthalen-1-ol (1.30 g, 2.26 mmol, 1.00 eq) in MeOH (8 mL) was added TsOH (7.78 mg, 0.05 mmol, 0.02 eq). The mixture was stirred at 75° C. for 2 min. Once TLC (PE:EA=5:1) showed the starting material was consumed completely, the mixture was concentrated in vacuum. The crude product was purified by silica gel column chromatography using 0-20% EtOAc/hexane to afford 7-(4-(6-(benzyloxy)-3,4-dihydronaphthalen-1-yl)-2-fluoro-5-methoxyphenyl)-2-(dimethoxymethyl)-7-azaspiro[3.5]nonane (1,20 g, 98.29% yield) as a yellow solid.

LC-MS purity: 96.2% (UV at 254 nm), 558.3 [M+H]⁺.

### Step 5:7-(4-(6-(benzyloxy)-2-bromo-3,4-dihydronaphthalen-1-yl)-2-fluoro-5-methoxyphenyl)-2-(dimethoxymethyl)-7-azaspiro[3.5]nonane

To a mixture of 7-(4-(6-(benzyloxy)-3,4-dihydronaphthalen-1-yl)-2-fluoro-5-methoxyphenyl)-2-(dimethoxymethyl)-7-azaspiro[3.5]nonane (1,20 g, 2.15 mmol, 1.00 eq) and DIEA (0.56 g, 4.30 mmol, 2.00 eq) in DMA (10 mL), was added pyridinium tribromide (0.83 g, 2.58 mmol, 1,2 eq) at 0° C. The mixture was stirred at 25° C. for 2 h. Once LC-MS showed the starting material was consumed completely, the mixture was poured into H₂O. The mixture was then extracted with EtOAc (3×50 mL), and the organic layer was washed with brine (3×50 mL), dried over Na₂SO₄, filtered and concentrated in vacuum. The residue was purified by silica gel column chromatography using 0-20% EtOAc/hexane to afford 7-(4-(7-(benzyloxy)-3-bromo-2H-chromen-4-yl)-2-fluoro-5-methoxyphenyl)-2-(dimethoxymethyl)-7-azaspiro[3.5]nonane (1.00 g, 73.01% yield) as a yellow solid.

LC-MS purity: 95,6% (UV at 254 nm), 637.2 [M+H]⁺.

### Steps 6:7-(4-(6-(benzyloxy)-2-phenyl-3,4-dihydronaphthalen-1-yl)-2-fluoro-5-methoxyphenyl)-2-(dimethoxymethyl)-7-azaspiro[3.5]nonane

To a mixture 7-(4-(7-(benzyloxy)-3-bromo-2H-chromen-4-yl)-2-fluoro-5-methoxyphenyl)-2-(dimethoxymethyl)-7-azaspiro[3.5]nonane (1.00 g, 1.57 mmol, 1.00 eq) in dioxane (16 mL) and H₂O (2 mL), were added phenylboronic acid (0.23 g, 1.89 mmol, 1,2 eq), K₂CO₃ (0.43 g, 3.14 mmol, 2.00 eq) and Pd(dppf)Cl₂ (0.11 g, 0.16 mmol, 0.1 eq). The mixture was stirred at 90° C. for 12 hours under Argon. Once LC-MS showed the starting material was consumed completely, the mixture was poured into H₂O, and extracted with EtOAc (3×50 mL). The combined organic layer was washed with brine (3×50 mL), dried over Na₂SO₄, filtered, and concentrated in vacuum. The crude product was purified by silica gel column chromatography using 0-30% EtOAc/hexane to afford 7-(4-(6-(benzyloxy)-2-phenyl-3,4-dihydronaphthalen-1-yl)-2-fluoro-5-methoxyphenyl)-2-(dimethoxymethyl)-7-azaspiro[3.5]nonane (0.95 g, 95.42% yield) as a yellow solid. LC-MS purity: 98.0% (UV at 254 nm), 634.3 [M+H]⁺.

### Steps 7:5-(4-(2-(dimethoxymethyl)-7-azaspiro[3.5]nonan-7-yl)-5-fluoro-2-methoxyphenyl)-6-phenyl-5,6,7,8-tetrahydronaphthalen-2-ol

To a mixture of 7-(4-(7-(benzyloxy)-3-phenyl-2H-chromen-4-yl)-2-fluoro-5-methoxyphenyl)-2-(dimethoxymethyl)-7-azaspiro[3.5]nonane (0.70 g, 1.5 mmol, 1.00 eq) in MeOH (10 mL) was added Pd/C(100 mg, 10% on crbon, wetted with c.a,55% water). The whole mixture was then stirred at room temperature overnight under H₂. Once the reaction finished, Pd/C was filtered, and the filtrate was concentrated in vacuum to afford 5-(4-(2-(dimethoxymethyl)-7-azaspiro[3.5]nonan-7-yl)-5-fluoro-2-methoxyphenyl)-6-phenyl-5,6,7,8-tetrahydronaphthalen-2-ol (0.70 g, 85.58% yield) as a white solid.

LC-MS purity: 97.2% (UV at 254 nm), 546.5 [M+H]⁺.

### Intermediate 21. 3-(7-oxo-5,7-dihydro-2H,6H-spiro[furo[2,3-f]isoindole-3,4′-piperidin]-6-yl)piperidine-2,6-dione hydrochloride

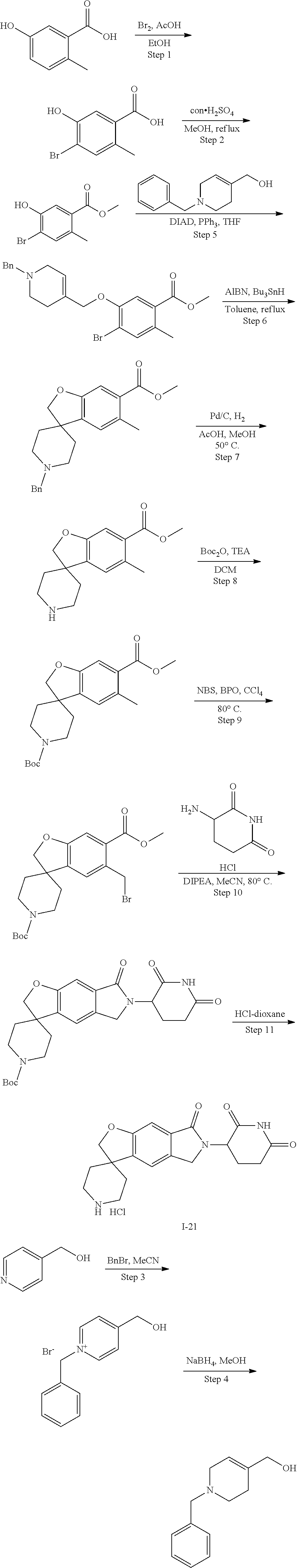

### Step 1:4-Bromo-5-Hydroxy-2-Methylbenzoic Acid

To a solution of 5-hydroxy-2-methylbenzoic acid (5.0 g, 32.9 mmol, 1.0 eq) in a mixture of ethanol (20 mL) and acetic acid (10 mL) was added dropwise bromine (3,4 mL, 65.7 mmol, 2.0 eq.). The reaction mixture was stirred for 10 h at room temperature, quenched with aqueous sodium thiosulfate solution (50 mL), and concentrated. The aqueous layer was extracted with ethyl acetate (50 mL× 3). The organic layer was dried over magnesium sulfate, filtered, and concentrated under reduced pressure to get crude 4-bromo-5-hydroxy-2-methylbenzoic acid (7.6 g, yield 100%) as a white solid. The crude product was directly used in next step without further purification. LC-MS (ESI): mass calcd, for C₈H₇BrO₃, 229.96; m/z found, 231,2 [M+H]⁺.

### Step 2: methyl 4-bromo-5-hydroxy-2-methylbenzoate

Con. H₂SO₄ (12 mL) was added to a suspension of 4-bromo-5-hydroxy-2-methylbenzoic acid (15 g, 65.72 mmol) in methanol (100 mL). The mixture was refluxed for 16 h. After evaporation, the residue was diluted with water (100 mL) and extracted with EA (100 mL×3). The organic layer was washed with H₂O (100 mL×2), saturated aqueous NaHCO₃ solution (100 mL×2) and brine (100 mL). The organic layer was dried over anhydrous Na₂SO₄, filtered and concentrated under reduced pressure. The residue purified by flash column chromatography on silica gel (PE/EA=4/1) to afford methyl 4-bromo-5-hydroxy-2-methylbenzoate (7.5 g, yield 47%) as a colorless solid. LC-MS(ESI): mass calcd, for C₉H₉BrO₃, 243.97; m/z found, 245.2 [M+H]⁺.

1HNMR (400 MHZ, CDCl₃) δ 7.56 (s, 1H), 7.36 (s, 1H), 5.52 (s, 1H), 3.88 (s, 3H), 2.50 (s. 3H),

### Step 3:1-benzyl-4-(hydroxymethyl)pyridin-1-ium bromide

To a solution of (pyridin-4-yl)methanol (8.9 g, 81.6 mmol, 1.0 eq) in CH₃CN(80 mL) was added a solution of (bromomethyl)benzene (11.705 mL, 97.9 mmol, 1,2 eq) in CH₃CN(40 mL). The reaction mixture was refluxed stirred at 90° C. for 3 h. After evaporation, the residue was washed with methyl tert-butyl ether, filtered, and dried to afford 1-benzyl-4-(hydroxymethyl)pyridin-1-ium bromide (16.33 g, yield 100%) as a yellow solid. LC-MS(ESI): mass calcd, for C₁₃H₁₄NO, 200.11; m/z found, 200.3 [M]⁺.

### Step 4: (1-benzyl-1,2,3,6-tetrahydropyridin-4-yl)methanol

To a solution of 1-benzyl-4-(hydroxymethyl)pyridin-1-ium bromide (16.3 g, 81,4 mmol, 1.0 eq) in CH₃OH (150 mL) was added NaBH₄ (9.3 g, 244.2 mmol, 3.0 eq) in portions at −20° C. The mixture was stirred at −20° C. for 1 h. The reaction was quenched with brine (100 mL) and extracted with EtOAc (200 mL×3). The organic layer was washed with brine (100 mL×3), dried over anhydrous Na₂SO₄, filtered and concentrated under reduced pressure. The residue was purified by flash column chromatography on silica gel (CH₃OH in DCM, from 0% to 10%) to afford (1-benzyl-1,2,3,6-tetrahydropyridin-4-yl)methanol (15 g, yield 91%) as a red oil. LC-MS (ESI): mass calcd, for C₁₃H₁₇NO, 203.13; m/z found, 204,4 [M+H]⁺.

¹H NMR (400 MHZ, DMSO-d₆) δ 7.24-7.18 (m, 4H), 7.16-7.12 (m, 1H), 5.43 (s, 1H), 4.61 (s, 1H), 3.71 (s, 2H), 3.42 (s, 2H), 2.76 (s, 2H), 2.39 (t. J=5,6 Hz, 2H), 1.91 (s, 2H), Step 5: methyl 5-[(1-benzyl-1,2,3,6-tetrahydropyridin-4-yl)methoxy]-4-bromo-2-methylbenzoate

To a solution of methyl 4-bromo-5-hydroxy-2-methylbenzoate (200 mg, 0.82 mmol, 1.0 eq), (1-benzyl-1,2,3,6-tetrahydropyridin-4-yl)methanol (166 mg, 0.82 mmol, 1.0 eq), and PPh₃ (321 mg, 1,22 mmol, 1.5 eq) in dry THF (10 mL) was added dropwise DIAD (0.25 mL, 1,22 mmol, 1.5 eq) at 0° C. under the N₂ atmosphere. The solution was stirred for 2 h. After evaporation, the residue was purified by flash column chromatography on silica gel (PE/EA=2/1 to 1/1) to afford methyl 5-[(1-benzyl-1,2,3,6-tetrahydropyridin-4-yl)methoxy]-4-bromo-2-methylbenzoate (300 mg, yield 85%) as a white solid. LC-MS(ESI): mass calcd, for C₂₂H₂₄BrNO₃, 429.09; m/z found, 431.30 [M+H]⁺.

### Step 6: methyl l′-(cyclohexylmethyl)-5-methyl-2H-spiro[1-benzofuran-3,4′-piperidine]-6-carboxylate

Tributyl tin hydride (0.5 mL, 1.84 mmol, 4.0 equiv) was added to a solution of methyl 5-[(1-benzyl-1,2,3,6-tetrahydropyridin-4-yl)methoxy]-4-bromo-2-methylbenzoate (200 mg, 0.46 mmol, 1.0 eq) and AIBN(15 mg, 0.09 mmol, 0.2 eq) in toluene (10 mL). The solution was refluxed in a sealed tube for 6 h. After cooled down to room temperature. The solution was quenched with saturated potassium fluoride solution (40 mL) and stirred at room temperature for 0.5 h. The mixture was extracted with EA (40 mL×3). The organic layer was washed brine (40 mL), dried over anhydrous Na₂SO₄, filtered and concentrated under reduced pressure. The residue was purified by prep-TLC (EA/PE=1/1) to afford methyl 1′-(cyclohexylmethyl)-5-methyl-2H-spiro[1-benzofuran-3,4′-piperidine]-6-carboxylate (20 mg, yield 43%) as a yellow solid. LC-MS (ESI): mass calcd, for C₂₂H₂₅NO₃, 351,18; m/z found, 352,30 [M+H]⁺.

¹H NMR (400 MHZ, CDCl₃) δ 7.37-7.27 (m, 6H), 6.99 (s, 1H), 4.37 (s, 2H), 3.85 (s, 3H), 3.54 (s, 2H), 2.89 (d, J=10.2 Hz, 2H), 2.52 (s, 3H), 2.10-1.95 (m, 4H), 1.70 (d, J=11.4 Hz, 2H), Step 7: methyl 5-methyl-2H-spiro[benzofuran-3,4′-piperidine]-6-carboxylate

A mixture of methyl l′-benzyl-5-methyl-2H-spiro[1-benzofuran-3,4′-piperidine]-6-carboxylate (1.0 g, 2.845 mmol, 1.0 eq), acetic acid (1 mL, 5.7 mmol, 6.1 eq), and 10% Pd/C(200 mg) in MeOH (20 mL) was stirred at 50° C. under H₂ (1 atm) for 3 h. After filtration, the filtrate was concentrated to get methyl 5-methyl-2H-spiro[benzofuran-3,4′-piperidine]-6-carboxylate (970 mg, yield 100%) as a colorless oil, which was directly used in the next step without further purification. LC-MS(ESI): mass calcd, for C₁₋₅H₁₉NO₃, 261,14; m/z found, 262.40 (M+H)⁺. Step 8: l′-(tert-butyl) 6-methyl 5-methyl-2H-spiro[benzofuran-3,4′-piperidine]-1′,6-dicarboxylate

To a stirred solution of methyl 5-methyl-2H-spiro[1-benzofuran-3,4′-piperidine]-6-carboxylate (970 mg, 3.7 mmol, 1.0 eq) and TEA (1 mL, 7.4 mmol, 2.0 eq) in DCM (10 mL) was added dropwise Boc₂O (0.8 mL, 3.7 mmol, 2.0 eq) at 0° C. The mixture was stirred at room temperature for 2 h. The reaction mixture was poured into water (10 mL) and extracted with DCM (30 mL× 2). The organic phase was dried over anhydrous Na₂SO₄ and concentrated under reduced pressure to afford 1′-(tert-butyl) 6-methyl 5-methyl-2H-spiro[benzofuran-3,4′-piperidine]-1′0.6-dicarboxylate (1,28 g, yield 100%) as a white solid. LC-MS(ESI): mass calcd, for C₂₀H₂₇NO₅, 361,19; m/z found, 306.4 [M+H-56]⁺.

### Step 9: l′-(tert-butyl) 6-methyl 5-(bromomethyl)-2H-spiro[benzofuran-3,4′-piperidine]-1′,6-dicarboxylate

A mixture of methyl 1′-benzyl-5-methyl-2H-spiro[1-benzofuran-3,4′-piperidine]-6-carboxylate (220 mg, 0.609 mmol, 1 eq), NBS(130 mg, 0.73 mmol, 1,2 eq), and BPO (60 mg, 0.243 mmol, 0.4 eq) in CCl₄ (10 mL) was refluxed for 4 h. After cooled to room temperature, the mixture was filtered, then the filtration was concentrated and to give l′-tert-butyl 6-methyl 5-(bromomethyl)-2H-spiro[1-benzofuran-3,4′-piperidine]-1′,6-dicarboxylate (100 mg, yield 37%) as a light-yellow solid. LC-MS(ESI): mass calcd, for C₂₀H₂₆BrNO₅, 439.10; m/z found, 462.20, [M+Na]⁺.

### Step 10: tert-butyl 6-(2,6-dioxopiperidin-3-yl)-7-oxo-6,7-dihydro-2H,5H-spiro[furo[2,3-flisoindole-3,4′-piperidine]-1′-carboxylate

DIPEA (0.12 mL, 0.681 mmol, 3.0 eq) was added to l′-tert-butyl 6-methyl 5-(bromomethyl)-2H-spiro[1-benzofuran-3,4′-piperidine]-1′,6-dicarboxylate (100 mg, 0.227 mmol, 1.0 eq) and 3-aminopiperidine-2,6-dione hydrochloride (56 mg, 0.341 mmol, 1.5 eq) in MeCN (5 mL) under nitrogen. The resulting suspension was stirred at 80° C. for 24 h. The reaction mixture was cooled to room temperature and filtered. The solid was washed with MeCN and purified by prep-TLC (100% EtOAc) to afford tert-butyl 6-(2,6-dioxopiperidin-3-yl)-7-oxo-6,7-dihydro-2H,5H-spiro[furo[2,3-f]isoindole-3,4′-piperidine]-1′-carboxylate (50 mg, yield 48%) as a white solid. LC-MS(ESI): mass calcd, for C₂₄H₂₉N₃O₆, 455.51; m/z found, 456.50. (M+H)⁺. Step 11:3-(7-oxo-5,7-dihydro-2H,6H-spiro[furo[2,3-f]isoindole-3,4′-piperidin]-6-yl)piperidine-2,6-dione

To a solution of tert-butyl 6-(2,6-dioxopiperidin-3-yl)-7-oxo-2,5,6,7-tetrahydrospiro[furo[2,3-f]isoindole-3,4′-piperidine]-1′-carboxylate (50 mg, 0.11 mmol, 1.0 eq) in DCM (1 mL) was added HCl-dioxane solution (4 M, 1 mL, 4 mmol, 36 eq) and the mixture was stirred for 30 min. After evaporation, the residue was purified by prep-HPLC with YMC-TA C18 (5 um, 20×250 mm), and mobile phase of 5-95% ACN in water over 10 min, and then hold at 100% ACN for 2 min, at a flow rate of 25 mL/min to get 3-{7-oxo-2,5,6,7-tetrahydrospiro[furo[2,3-f]isoindole-3,4′-piperidine]-6-yl}piperidine-2,6-dione hydrochloride (30 mg, yield 70%) as a white solid. LC-MS(ESI): mass calcd, for C₁₉H₂₁N₃O₄, 355.19; m/z, found, 356.20 [M+H]⁺.

¹H NMR (400 MHZ, DMSO-d₆) δ 10.98 (s, 1H), 8.78 (s, 2H), 7.36 (s, 1H), 7.06 (s, 1H), 5.11-5.06 (m, 1H), 4.58 (s, 2H), 4.38 (d, J=17.0 Hz, 1H), 4.25 (d, J=17.0 Hz, 1H), 3.30-3,27 (m, 2H), 3.04-2.92 (m, 2H), 2.93-2.84 (m, 1H), 2.62-2.56 (m, 1H), 2.44-2.29 (m, 1H), 2.09-1.97 (m, 3H), 1.90-1.79 (m, 2H),

### Intermediate 22, 3-((4-(7,7-difluoro-3-azabicyclo[4.1.0]heptan-6-yl)phenyl)amino)piperidine-2,6-dione hydrochloride salt

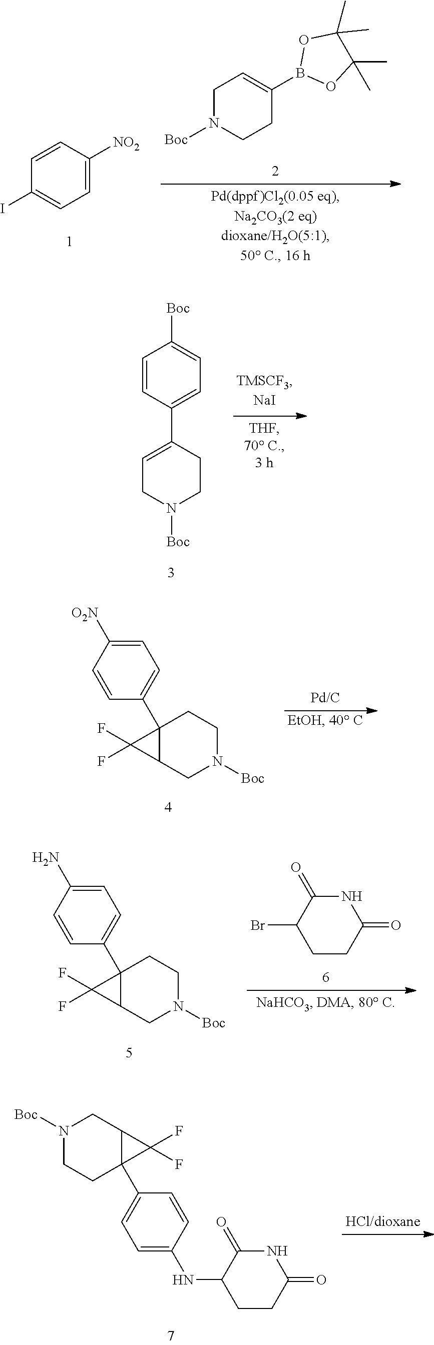

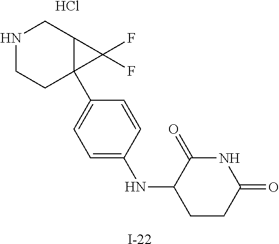

### Step 1: tert-butyl 4-(4-nitrophenyl)-3,6-dihydropyridine-1 (2H)-carboxylate

To a mixture of 1-iodo-4-nitrobenzene (10 g, 40.1 mmol 1 eq.) and tert-butyl 4-(4,4,5,5-tetramethyl-1,3,2-dioxaborolan-2-yl)-3,6-dihydropyridine-1 (2H)-carboxylate (14.9 g, 48.2 mmol, 1,2 eq.) in dioxane (100 mL)/H₂O (20 ml) was added Pd(pddf)Cl₂ (1.9 g, 2.0 mmol, 0.05 eq.) and Na₂CO₃ (12.8 g, 120.5 mmol, 3 eq.) under Ar. The reaction mixture was stirred at 50° C. under N₂ for 16 h. The mixture was concentrated and purified by column chromatography on silica gel eluted with 0-20% EtOAc/hexane to afford tert-butyl 4-(4-nitrophenyl)-3,6-dihydropyridine-1 (2H)-carboxylate (6.7 g, 54% yield) as white solid.

### Step 2: tert-butyl 7,7-difluoro-6-(4-nitrophenyl)-3-azabicyclo[4.1.0]heptane-3-carboxylate

To a mixture of tert-butyl 4-(4-nitrophenyl)-3,6-dihydropyridine-1 (2H)-carboxylate (600 mg, 2 mmol 1 eq.) and Nal (14.9 g, 48.2 mmol, 1,2 eq.) in THF (10 mL) was added Pd(pddf)Cl₂ (150 mg, 1 mmol, 0.5 eq.) followed by TMSCF3 (1,4 g, 10 mmol, 5 eq.). The reaction mixture was stirred at 70° C. under N₂ for 3 h. The mixture was concentrated and purified by column chromatography on silica gel eluted with 0-20% EtOAc/hexane to afford tert-butyl 7,7-difluoro-6-(4-nitrophenyl)-3-azabicyclo[4.1.0]heptane-3-carboxylate (700 mg, crude) as brown oil. Step 3: tert-butyl 6-(4-aminophenyl)-7,7-difluoro-3-azabicyclo[4.1.0]heptane-3-carboxylate

To a mixture of tert-butyl 7,7-difluoro-6-(4-nitrophenyl)-3-azabicyclo[4.1.0]heptane-3-carboxylate (400 mg, 1,1 mmol, 1 eq.) in EtOH (10 mL) was added Pd/C(100 mg). The reaction mixture was stirred at 40° C. for 16 h under H₂. The catalyst was removed by filtration and the filtrate was concentrated to afford tert-butyl 6-(4-aminophenyl)-7,7-difluoro-3-azabicyclo[4.1.0]heptane-3-carboxylate (360 mg, crude) as yellow oil.

LC-MS purity: 98.1% (UV at 254 nm), 269.1 [M+H-56]⁺.

### Step 4: tert-butyl 6-(4-((2,6-dioxopiperidin-3-yl)amino)phenyl)-7,7-difluoro-3-azabicyclo[4.1.0]heptane-3-carboxylate

To a mixture of tert-butyl 6-(4-aminophenyl)-7,7-difluoro-3-azabicyclo[4.1.0]heptane-3-carboxylate (970 mg, 3 mmol 1.0 eq.) in DMA (8 mL) was added 3-bromopiperidine-2,6-dione (570 mg, 3 mmol 1.0 eq.) and NaHCO₃ (251 mg, 3 mmol, 1.0 eq.). The mixture was stirred at 80° C. overnight and cooled to room temperature. The mixture was concentrated and the residue was purified by column chromatography on silica gel eluted with 0-100% EtOAc/hexane to afford tert-butyl 6-(4-((2,6-dioxopiperidin-3-yl)amino)phenyl)-7,7-difluoro-3-azabicyclo[4.1.0]heptane-3-carboxylate as yellow solid (1 g, 65% yield).

LC-MS purity: 100% (UV at 254 nm), 436.0 [M+H]⁺.

### Step 5:3-((4-(7,7-difluoro-3-azabicyclo[4.1.0]heptan-6-yl)phenyl)amino)piperidine-2,6-dione hydrochloride salt

A mixture of tert-butyl 6-(4-((2,6-dioxopiperidin-3-yl)amino)phenyl)-7,7-difluoro-3-azabicyclo[4.1.0]heptane-3-carboxylate (40 mg, 1.0 eq) in HCl/dioxane (2 mL) was stirred at 20° C. for 2 h. The after reaction was direct concentration as to give 3-((4-(7,7-difluoro-3-azabicyclo[4.1.0]heptan-6-yl)phenyl)amino)piperidine-2,6-dione hydrochloride salt as white solid (40 mg, crude).

LC-MS purity: 100% (UV at 254 nm), 336.1 [M+H]⁺.

### Intermediate 23: (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione hydrochloride salt

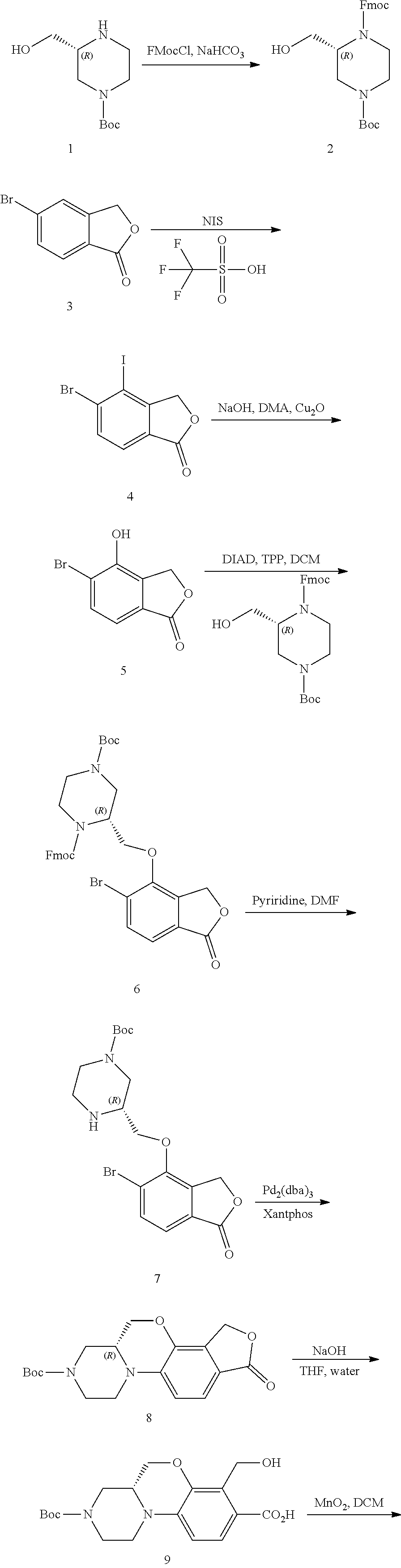

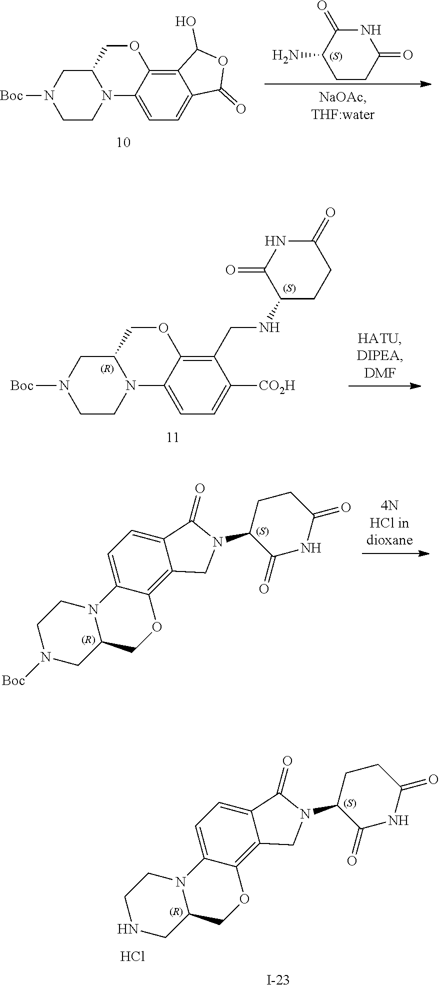

### Step 1:1-((9H-fluoren-9-yl)methyl) 4-(tert-butyl) (R)-2-(hydroxymethyl)piperazine-1,4-dicarboxylate

(R)-1-Boc-3-(Hydroxymethyl)piperazine (1, 10 g, 46.2 mmol) was dissolved in a mixture of DCM (180 mL) and sat. NaHCO₃ (180 mL). FMocCl (46.2 mmol) was dissolved in DCM (15 mL) and added dropwise with vigorous stirring. The mixture was stirred for 1 hour. The layers were separated and the aqueous phase was extracted with DCM. The combined organic phases were washed with brine, dried over Na₂SO₄ and the solvent was removed in vacuo to give an oil. The crude product was purified by silica gel column chromatography using 0-80% EtOAc/hexane (80% yield).

### Step 2:5-bromo-4-iodoisobenzofuran-1 (3H)-one

To a solution of 5-Bromo-3H- isobenzofuran-1-one (3, 5 g, 23,4 mmol, 1 eq.) in trifluoromethancsulfonic acid (68 g, 40 mL, 19.30 eq) was added NIS(5.5 g, 24.6 mmol, 1.05 eq.) at 0° C. in portions. The mixture was allowed to warm to room temperature and stirred overnight. TLC (hexane:ethyl acetate=5:1) showed no starting material remained and two new spots (R_{f}=0.4, 0.5) formed. The reaction mixture was poured into ice-water (100 mL) and yellow solid precipitated. The mixture was filtered and the filter cake was washed with ice cold water. The filter cake was dissolved in DCM (500 mL) and washed with 1 (M)Na₂S203 followed by dried over sodium sulfate. The mixture was filtered and the filtrate was concentrated to afford a yellow solid. The crude product was purified on a 120 g silica column running a 0-10% EtOAc/hexane gradient over 70 min, ¹H NMR (400 MHZ, CDCl₃): δ 7.83 (d, J=8.0 Hz, 1H), 7.77 (d, J=8.0 Hz, 1H), 5.10 (s, 2H),

### Step 3:5-bromo-4-hydroxyisobenzofuran-1 (3H)-one

To a mixture of 5-Bromo-4-iodo-3H- isobenzofuran-1-one (4, 4 g, 1 eq), sodium hydroxide (2,3 g, 5 eq) in water (40 mL, 1.5 M) and N,N-dimethylacetamide (20 ml) was added cuprous oxide (0.338 g, 0.2 eq). The reaction mixture was heated to 80° C. and held for 12 h. TLC (Hexane; ethyl acetate=1:1, R_{f}=0.3) showed the reaction was completed. The reaction mixture neutralized using 1 (N) hydrochloride solution and extracted with ethyl acetate (40 mL×2), washed with brine (150 mL), and then dried over sodium sulfate. The crude product was purified by silica gel column chromatography using 0-100% EtOAc/hexane, 5-Bromo-4-hydroxy-3H-isobenzofuran-1-one (5, 50% yield) was obtained as a white solid, ¹H NMR (400 MHZ, DMSO) 8 10.90 (s, 1H), 7.72 (d, J=8.0 Hz, 1H), 7.23 (d, J=8.0 Hz, 1H), 5.35 (s, 2H),

### Step 4. 1-((9H-fluoren-9-yl)methyl) 4-(tert-butyl) (R)-2-(((5-bromo-1-oxo-1,3 dihydroisobenzofuran-4-yl)oxy)methyl)piperazine-1,4-dicarboxylate

To a solution of 5-Bromo-4-hydroxyisobenzofuran-1 (3H)-one (5, 700 mg, 3 mmol, 1 eq.) in 12 mL of THF/DCM, 1-((9H-fluoren-9-yl)methyl) 4-(tert-butyl) (R)-2-(hydroxymethyl)piperazine-1,4-dicarboxylate (2 gm, 4.5 mmol, 1.5 eq.) and PPh₃ (1,17 gm, 4.5 mmol, 1.5 eq.) was added. The reaction mixture was cooled to 0° C. and DIAD (0.9 mL, 4.5 mmol, 1.5 eq.) was added dropwise. The resultant mixture was then stirred overnight at room temperature. The solvent was evaporated at reduced pressure; the crude product was purified by silica gel column chromatography using 0-100% EtOAc/hexane. LC/MS(ESI) m/z: 649.15

### Step 5: tert-butyl (R)-3-(((5-bromo-1-oxo-1,3-dihydroisobenzofuran-4-yl)oxy)methyl)piperazine-1-carboxylate

To a solution of 1-((9H-fluoren-9-yl)methyl) 4-(tert-butyl) (R)-2-(((5-Bromo-1-oxo-1,3-dihydroisobenzofuran-4-yl)oxy)methyl)piperazine-1,4-dicarboxylate (6, 1 gm) was added 20% (v/v)piperidine in DMF (5 mL/gm of SM). The resulting mixture was stirred at room temperature overnight. The mixture was diluted with ethyl acetate and washed with water. The combined organic phases were washed with brine, dried over Na₂SO₄ and the solvent was removed in vacuo to give an oil. The crude product was purified by silica gel column chromatography using 0-5% DCM in methanol. Yield 70%. LC/MS(ESI) m/z: 426.08 [M+1]⁺.

### Step 6: tert-butyl (R)-1-oxo-1,3,5a,6,8,9-hexahydroisobenzofuro[4,5-b]pyrazino[1,2-d][1,4]oxazine-7 (5H)-carboxylate

A vial was charged with tert-butyl (R)-3-(((5-bromo-1-oxo-1,3-dihydroisobenzofuran-4-yl)oxy)methyl)piperazine-1-carboxylate (7, 170 mg, 0.38 mmol, 1 eq.), Pd₂(dba) 3 (0.1 eq.), XantPhos (0.2 eq.), Cs₂CO₃ (3 eq.) and dioxane (5 mL). The mixture was purged with nitrogen and heated to 100° C. for 6 h. TLC (ethyl acetate:petroleum ether=1:2) showed reaction was complete. The mixture was diluted with ethyl acetate and washed with water. The organic layer was washed with brine and dried over sodium sulfate. The crude product was purified by silica gel column chromatography using 0-50% EtOAc/hexane. LC/MS(ESI) m/z: 347.15 [M+1]⁺. Yield 60%

### Step 7: (R)-3-(tert-butoxycarbonyl)-7-(hydroxymethyl)-1,2,3,4,4a,5-hexahydrobenzo[b]pyrazino[1,2-d][1,4]oxazine-8-carboxylic acid

To a solution of tert-butyl (R)-1-oxo-1,3,5a,6,8,9-hexahydroisobenzofuro[4,5-b]pyrazino[1,2-d][1,4]oxazine-7 (5H)-carboxylate (8, 346 mg, 1 mmol, 1 eq) in tetrahydrofuran (4 mL) and water (4 mL) was added sodium hydroxide (200 mg, 5 eq). The mixture was stirred at 20° C. for 16 h. TLC (ethyl acetate: hexane=1:1) showed reaction was complete. The mixture was adjusted to pH=5 with aq, hydrochloric acid (1 M) and extracted with ethyl acetate (10 mL×3). The organic layer was washed with brine (10×2 mL) and dried over sodium sulfate. The crude material was not further purified and used as crude for the next steps. LC/MS(ESI) m/z: 365.16

### Step 8: tert-butyl (5aR)-3-hydroxy-1-oxo-1,3,5a,6,8,9-hexahydroisobenzofuro[4,5-b]pyrazino[1,2-d][1,4]oxazine-7 (5H)-carboxylate

To a solution of (R)-3-(tert-butoxycarbonyl)-7-(hydroxymethyl)-1,2,3,4,4a,5-hexahydrobenzo[b]pyrazino[1,2-d][1,4]oxazine-8-carboxylic acid (9, 1 eq.) in dichloromethane (10 mL) was added manganese dioxide (15 eq.). The mixture was stirred at 20° C. for overnight. TLC (ethyl acetate: hexane=1:1) showed reaction was complete. The mixture was diluted with dichloromethane (10 mL) and filtered through a pad of Celite. The filtrate was concentrated in vacuum. The crude product was purified by silica gel column chromatography. LC/MS(ESI) m/z: 363.16. 1H NMR (400 MHZ, CD₃OD) δ 7.32 (d, J=8.3 Hz, 1H), 7.14 (d, J=8.4 Hz, 1H), 6.64-6.40 (m, 1H), 4.42 (dd, J=11.0, 3.0 Hz, 1H), 4.23-4.01 (m, 3H), 3.95 (d, J=12.4 Hz, 1H), 3.34-3,23 (m, 1H), 3.08 (brs, 1H), 2.87 (td, J=12.2, 3.5 Hz, 1H), 2.74 (s, 1H) 1.50 (S, 9H),

### Step 9: (R)-3-(tert-butoxycarbonyl)-7-((((S)-2,6-dioxopiperidin-3-yl)amino)methyl)-1,2,3,4,4a,5-hexahydrobenzo[b]pyrazino[1,2-d][1,4]oxazine-8-carboxylic acid

To a mixture of(S) 3-aminopiperidine-2,6-dione (10, 1.5 eq., HCl salt) in methanol (2 ml) and dichloromethane (4 ml) was added sodium acetate (4 eq.). The mixture was stirred at 20° C. for 15 min, then tert-butyl (5aR)-3-hydroxy-1-oxo-1,3,5a,6,8,9-hexahydroisobenzofuro[4,5-b]pyrazino[1,2-d][1,4]oxazine-7 (5H)-carboxylate (1 eq.) was added and the mixture was stirred for 30 mins. Sodium cyanoborohydride (2 eq.) was added and the mixture was further stirred for 1 hour. LCMS showed the reaction was complete. The mixture was adjusted to pH=4-5 with an aqueous hydrochloric acid solution (1 M) and extracted with ethyl acetate (10 mL×3). The crude material was not further purified and used as crude for the next steps. LC/MS(ESI) m/z: 475.21 Step 10: tert-butyl (R)-2-((S)-2,6-dioxopiperidin-3-yl)-1-oxo-2,3,5a,6,8,9-hexahydro-1H-pyrazino[1′,2′: 4,5][1,4]oxazino[2,3-e]isoindole-7 (5H)-carboxylate

To a solution of (R)-3-(tert-butoxycarbonyl)-7-((((S)-2,6-dioxopiperidin-3-yl)amino)methyl)-1,2,3,4,4a,5-hexahydrobenzo[b]pyrazino[1,2-d][1,4]oxazine-8-carboxylic acid (11, 90 mg, 0.18 mmol, 1 eq.) in dimethylformamide (5 mL) was added HATU (72 mg, 1.0 eq.) followed by addition of DIPEA (3 eq.). The solution was stirred for 15 mins, at 0° C. The residue was purified by reverse phase HPLC to get the desired compound 12. LC/MS(ESI) m/z: 457.20.

¹H NMR (400 MHZ, Methanol-d₄) δ 7.32 (d, J=8.3 Hz, 1H), 7.08 (d, J=8.4 Hz, 1H), 5.10 (dd, J=13.3. 5.2 Hz, 1H), 4.46-4.30 (m, 3H), 4.23-3.98 (m, 3H), 3.93 (d, J=12.4 Hz, 1H), 3.22 (ddd, J=11,2, 8.2, 3.0 Hz, 1H), 3.07 (s, 1H), 2.99-2.61 (m, 4H), 2.59-2.42 (m, 1H), 2.21-2.07 (m, 1H), 1.51 (s, 9H).

### Step 11: (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione hydrochloride

A mixture of tert-butyl (R)-2-((S)-2,6-dioxopiperidin-3-yl)-1-oxo-2,3,5a,6,8,9-hexahydro-1H-pyrazino[1′,2′: 4,5][1,4]oxazino[2,3-e]isoindole-7 (5H)-carboxylate (456 mg, 1.0 mmol, 1 eq.) in HCl/dioxane (10 mL) was stirred at room temperature for 2 h. The reaction mixture was concentrated to afford (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione hydrochloride (1-23, 400 mg, crude) as white solid.

### Intermediate 24:3-((R)-8-oxo-1,2,3,4,4a,5,8,10-octahydro-9H-pyrazino[1′,2′: 4,5][1,4]oxazino[2,3-f]isoindol-9-yl)piperidine-2,6-dione trifluoroacetat salt

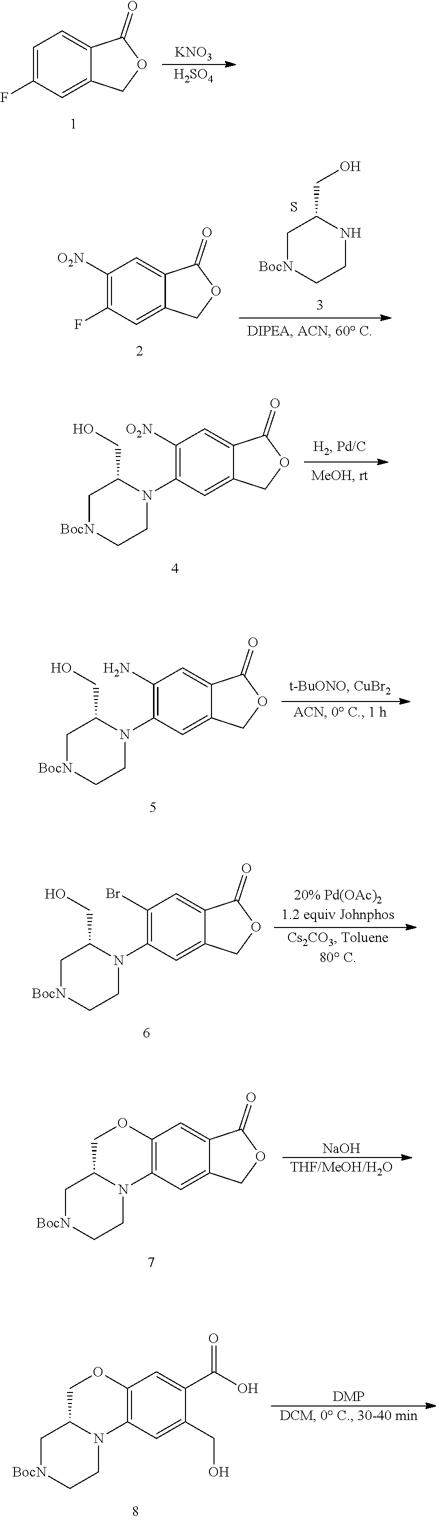

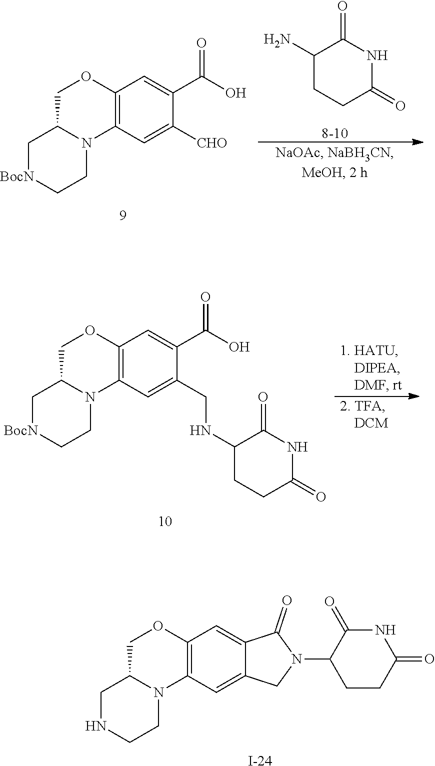

Intermediate I-24 was made using the similar procedure for making intermediate I-10. LC-MS: [M+H]⁺=356.91. 1H NMR (400 MHZ, Methanol-d₄) δ 7.17 (s, 1H), 7.13 (d, J=1.7 Hz, 1H), 5.14-5.03 (m, 1H), 4.44-4,28 (m, 3H), 4.27-4.17 (m, 1H), 4.12-4.03 (m, 1H), 3.61-3.42 (m, 3H), 3.30-3,21 (m, 1H), 3.21-3.11 (m, 1H), 3.04-2.95 (m, 1H), 2.95-2.83 (m, 1H), 2.81 -2,72 (m, 1H), 2.52-2.38 (m, 1H), 2.19-2.10 (m, 1H), 13C NMR (101 MHZ, MeOD) δ 174.66, 172.46, 172.44, 171.64, 171.61, 146.51, 146.47, 139.24, 139.20, 138.14, 123.98, 123.91, 111.93, 108.73, 108.70, 66.97, 53.71, 53.60, 50.88, 50.86, 44.38, 44.24, 44.20, 43.62, 32,38, 24.13.

### Intermediate 25:3-((S)-6-methyl-8-oxo-2,3,4,4a,5,6,8,10-octahydropyrazino[1,2-a]pyrrolo[3,4-g]quinoxalin-9 (1H)-yl)piperidine-2,6-dione trifluoroacetat salt

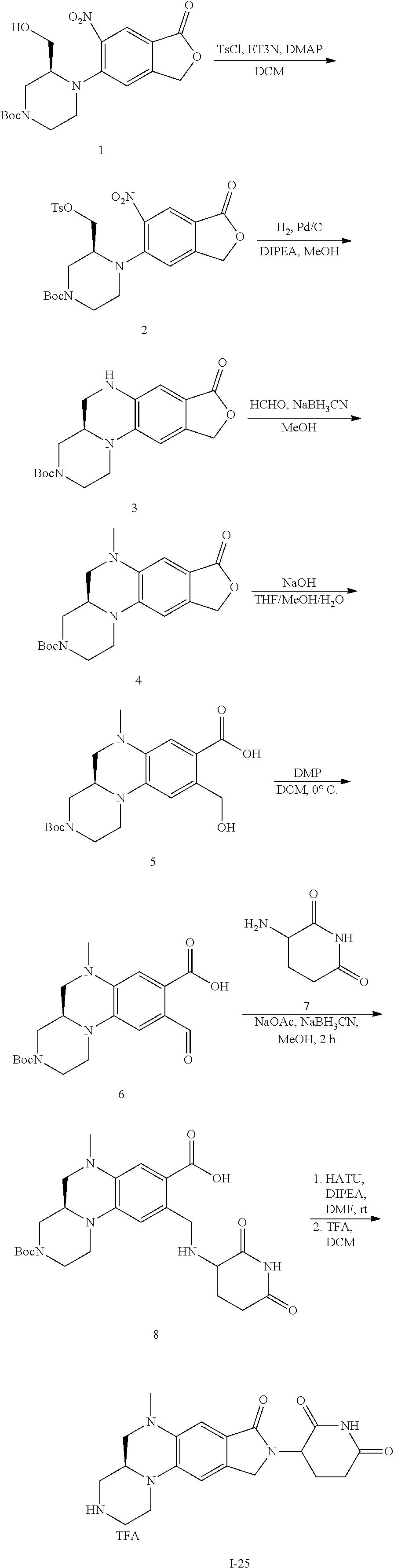

### Step 1: tert-butyl(S)-4-(6-nitro-1-oxo-1,3-dihydroisobenzofuran-5-yl)-3-((tosyloxy)methyl)piperazine-1-carboxylate

To a solution of 1 (1 equiv, 1,49 g) in DCM (30 mL) was TsCl (2.0 equiv, 1,44 g), Et₃N (4.0 equiv, 2.11 mL) and DMAP (0.2 equiv, 92 mg), and the mixture was stirred at rt overnight. TLC (n-Hexane: EA=1:1) indicated the starting material 1 was completely conversion and an new spot detected. Then the reaction mixture was diluted with DCM, washed with brine, dried over Na₂SO₄, and concentrated under reduced pressure to give a residue which was purified by silica gel flash chromatography (n-Hexane: EA=100:0 to 60:40). The desired product 2 was obtained as a yellow foam (1.67 g, yield=81%).

### Step 2, tert-butyl (R)-8-oxo-1,2,4,4a,5,6,8,10-octahydro-3H-furo[3,4-g]pyrazino[1,2-a]quinoxaline-3-carboxylate

To a solution of 2 (1.0 equiv, 1.67 g) in MeOH (20 mL) was added DIPEA (2.0 equiv, 1.06 mL), followed by Pd/C(0.5 equiv, 835 mg). The reaction mixture was degassed and purged with H₂ three times and keep stirred at rt overnight. UPLC-MS showed the starting material completely conversed to desired product 3. Then the reaction mixture was filtered through Celite, and the filtrate was concentrated under reduced pressure to give a residue which was purified by silica gel flash chromatography (DCM: McOH=100:0 to 95:5). The desired product 3 was obtained as a yellow solid (957 mg, yield=91%).

### Step 3: tert-butyl (R)-6-methyl-8-oxo-1,2,4,4a,5,6,8,10-octahydro-3H-furo[3,4-g]pyrazino[1,2-a]quinoxaline-3-carboxylate

To a solution of 3 (1.0 equiv, 410 mg) in MeOH/AcOH/DCM (10 mL/1 mL/3 mL) was added HCHO (5.0 equiv, 470 mg), and the mixture was kept stirring for 2 h. Then NaBH₃CN(5.0 equiv, 361 mg) was added, 15 min Later, UPLC-MS showed the starting material 3 all converted to desired product 4. The reaction mixture was concentrated under reduced pressure, diluted with DCM, washed with brine, dried over Na₂SO₄ and concentrated to give a yellow powder which is directly used in the next step.

### Step 4: (R)-3-(tert-butoxycarbonyl)-9-(hydroxymethyl)-6-methyl-2,3,4,4a,5,6-hexahydro-1H-pyrazino[1,2-a]quinoxaline-8-carboxylic acid

4 (1.0 equiv, 427 mg) was dissolved in THE/MeOH/H₂O (3 mL/3 mL/1 mL), and NaOH (5.0 equiv, 238 mg) was added. The reaction was kept stirring at 40° C. overnight. Then the reaction mixture was concentrated under reduced pressure to remove the solvent. The residue was diluted with 3-4 mL H₂₀, followed by acidified with 2 N aq. HCl to PH 3-4. White solid was precipitated, which was collected and dried to give desired product 5 as a white powder 358 mg (yield=80% in two steps).

### Step 5: (R)-3-(tert-butoxycarbonyl)-9-formyl-6-methyl-2,3,4,4a,5,6-hexahydro-1H-pyrazino[1,2-a]quinoxaline-8-carboxylic acid

To a solution of 5 (1.0 equiv, 305 mg) in DCM (20 mL) was added DMP (1.65 equiv, 565 mg) into 3 potions at 0° C. 30 min Later, UPLC-MS indicated that 5 was completely conversion and a new main peak with desired MS formed, then the reaction was immediately diluted with DCM, washed with brine, dried over and concentrated under reduced pressure to give a crude product 6 which is directly used in the next step.

### Step 6: (4uR)-3-(tert-butoxycarbonyl)-9-(((2,6-dioxopiperidin-3-yl)amino)methyl)-6-methyl-2,3,4,4a,5,6-hexahydro-1H-pyrazino[1,2-a]quinoxaline-8-carboxylic acid

A mixture of 6 (1.0 equiv, 303 mg), 7 (1.5 equiv, 199.5 mg) and NaOAc (1.5 equiv, 99.4 mg) was dissolved in MeOH (20 mL), and kept stirring at rt for 20 min. Then NaBH₃CN(3.0 equiv, 151 mg) was added in 3 potions, 2 h Later, UPLC-MS showed the starting material 6 was completely conversion and a new main peak with desired MS formed. Next, the reaction mixture was quenched with 4 mL water and concentrated under reduced pressure to give a residue which was purified by pre-HPLC(20% to 100% acetonitrile (0.1% HCOOH, not TFA) in 80 min, 60 mL/min, 27% acetonitrile come out). The desired product 8 was obtained as a dark solid 138 mg (yield=35% in two steps) after lyophilization.

### Step 7:3-((S)-6-methyl-8-oxo-2,3,4,4a,5,6,8,10-octahydropyrazino[1,2-a]pyrrolo[3,4-g]quinoxalin-9 (1H)-yl)piperidine-2,6-dione trifluoroacetate salt

To a solution of 8 (1.0 equiv, 138 mg) in DMF (5 mL) was added HATU (1,1 equiv, 118 mg) and DIPEA (3.0 equiv, 148 uL), and the reaction was stirred at rt for 20-30 min. UPLC-MS indicated a new main peak with desired MS formed, then quenched with 3 mL water and purified by HPLC-MS(acetonitrile 35% to 100% in 65 min, 60 mL/min, 44% acetonitrile come out). Collected the solution and concentrated to give a solid which was dissolved into TFA/DCM to deprotect the Boc group. The title compound I-25 was obtained as a light purple solid 40 mg (yield is much higher than here because much product was lost when purified) after removed the solvent and lyophilized. LC-MS: [M+H]⁺=370.02. 1H NMR (400 MHZ, Methanol-d₄) δ 6.99-6.93 (m, 2H), 5.12-5.04 (m, 1H), 4.34-4,29 (m, 1H), 4.27-4.18 (m, 1H), 3.69-3.60 (m, 1H), 3.51-3.40 (m, 2H), 3.39-3.33 (m, 1H), 3.27-3.12 (m, 4H), 3.07-3.00 (m, 1H), 2.92 (s, 3H), 2.88-2.85 (m, 1H), 2.80-2,73 (m, 1H), 2.51-2.38 (m, 1H), 2.18-2.10 (m, 1H),

### Intermediate 26: (3S)-3-(4-methyl-1-oxo-3,4,5,5a,6,7,8,9-octahydropyrazino[1,2-a]pyrrolo[3,4-f]quinoxalin-2 (1H)-yl)piperidine-2,6-dione trifluoroacetate salt

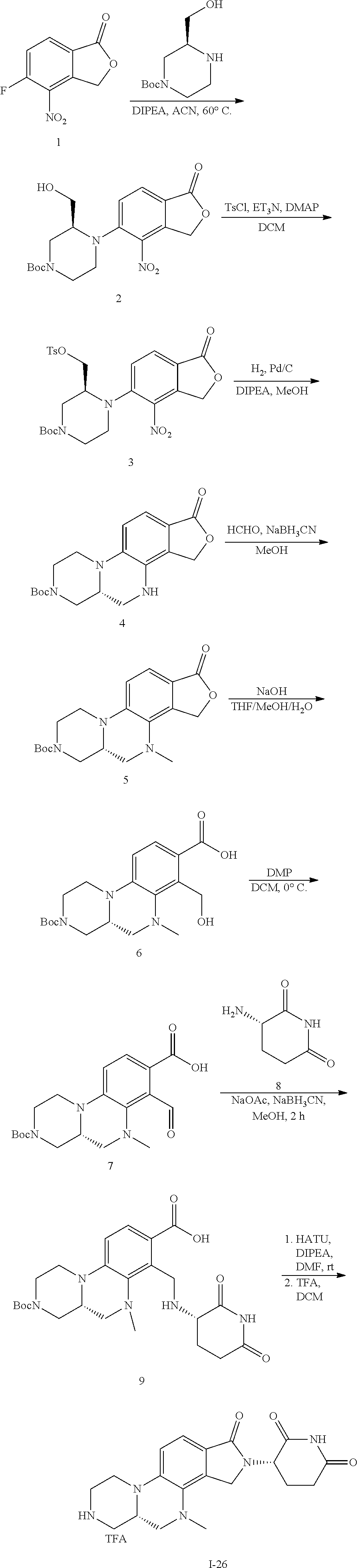

Intermediate I-26 was made using the similar procedure for making intermediate I-25. LC-MS: [M+H]⁺=370.28. 1H NMR (400 MHZ, Methanol-d₄) δ 7.32 (dd, J=8.4, 2,3 Hz, 1H), 7.09 (dd, J=8.6, 2.5 Hz, 1H), 5.14-5.04 (m, 1H), 4.63-4,44 (m, 2H), 4.30-4.16 (m, 1H), 3.60-3.51 (m, 1H), 3.49-3.39 (m, 2H), 3.29-3,21 (m, 2H), 3.16-3.02 (m, 2H), 2.98-2.83 (m, 5H), 2.82-2,72 (m, 1H), 2.58-2.45 (m, 1H), 2.21-2.10 (m, 1H), 13C NMR (101 MHZ, MeOD) δ 174.70, 172.53, 172.50, 171.69, 171.63, 141.77, 141.73, 133.94, 133.82, 133.63, 124.97, 118.36, 118.32, 115.17, 53.85, 53.82, 53.67, 53.63, 47.98, 47.86, 46.28, 44.83, 44.39, 43.81, 43.75, 32,37, 24.03, 23.99.

### Intermediate 27:3-(7′-oxo-2′,3′,7′,9′-tetrahydro-8′H-spiro[piperidine-4,4′-pyrano[2,3-e]isoindol]-8′-yl)piperidine-2,6-dione trifuluoroacetate salt

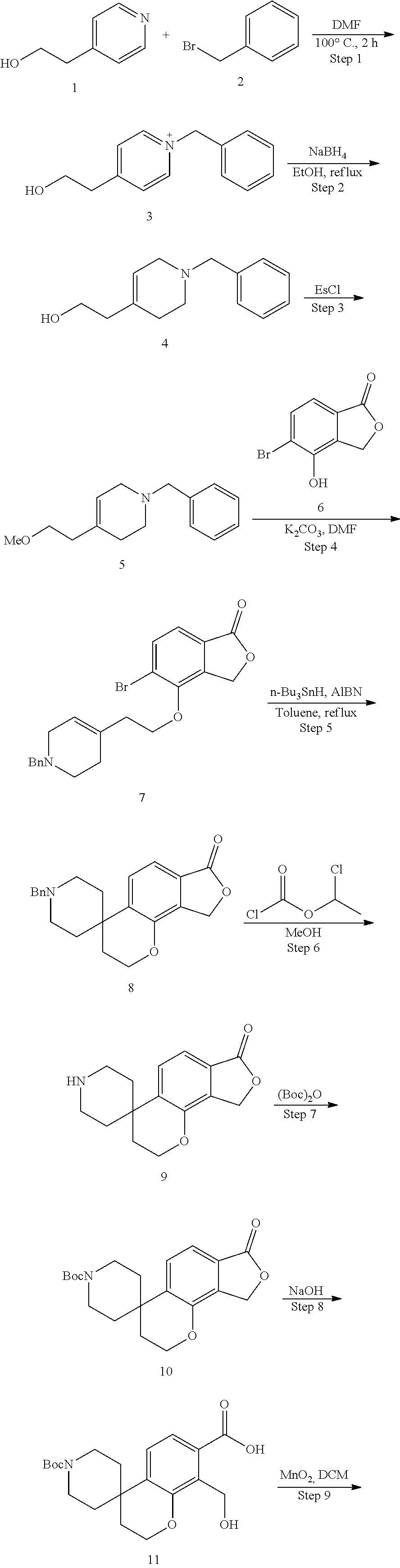

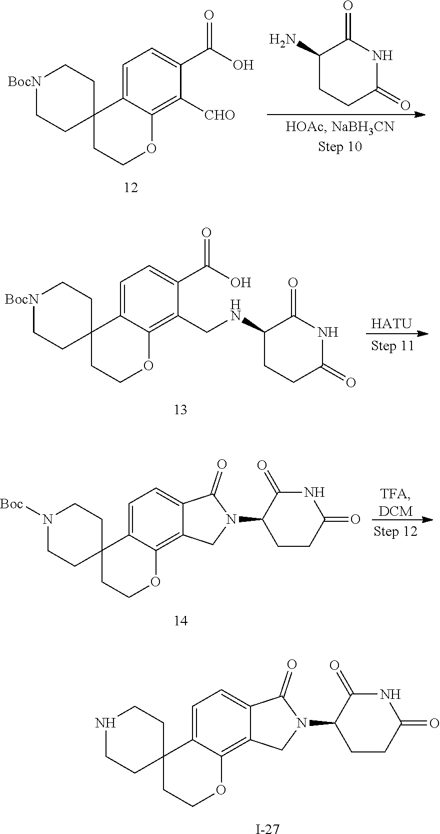

### Step 1-2

To a solution of 2-(pyridin-4-yl) ethan-1-ol (1, 10 g, 91.6 mmol, 1.0 eq.) in DMF (40 mL) was added BnBr (15.3 g, 108 mmol, 1,1 eq.). The mixture was allowed to heat to 100° C. and stirred 3 h. TLC showed no starting material remained and a new spot formed. The residue was dissolved in EtOH (150 mL), then 4.0 g of sodium borohydride (119.1 mmol, 1.3 eq.) was added portionwise at 0° C. The mixture was continued to stir at 0° C. for 1 h and then at reflux for 2 h. The solvent was evaporated under reduced pressure, then water was added, and the mixture was extracted with EA. The combined organic phases were dried over Na₂SO₄ and evaporated. The residue was purified by flash chromatograph (DCM:MeOH=100:0-30:1) to afford 10 g of product 4 (Viscous oil, 2 steps, yield 56%). LC-MS: 218 [M+H]⁺.

### Step 3

To a solution of compound 4 (10 g, 1 eq.) in DCM (200.0 mL) was added DMAP (0.1 eq.) and TEA (2 eq.) at 0° C. Then EsCl (1.5 eq.) was slowly added into and the mixture was stirred at R.T, for 1 h. The reaction was partitioned between EtOAc and water. The combined organic layers were dried over anhydrous Na₂SO₄, filtered, and concentrated in vacuo. The crude residue was purified by flash chromatograph to give compound 5 as a yellow solid (10 g, yield 70%). LC-MS: 310 [M+H]⁺.

### Step 4

To a solution of compound 6 (10 g, 1.0 eq.) in 100 mL of DMF, compound 5 (16.2 g, 1,2 eq.) and K₂CO₃ (1.6 eq.) was added. The reaction mixture was heated to 70° C. and stirred overnight. The reaction mixture was poured into ice-water and extracted with ethyl acetate, washed with brine, and then dried over sodium sulfate. The solvent was evaporated at reduced pressure and the crude product was purified by silica gel column chromatography using 0-100% EtOAc/hexane. The desired product 7 was obtained as a yellow foam (11 g, yield 60%), 428/430 [M+H]⁺.

### Step 5

To a solution of 7 (5 g, 1.0 eq.) in toluene (50 mL) was added n-BusSnH (13.6 g, 4.0 eq.) and AIBN(0.4 g, 0.1 eq.). The mixture was heated to reflux and stirred overnight. TLC (PE:EA=1:1) showed no starting material remained and new spots formed. The reaction mixture was poured into saturated aq. KF solution (100 mL) and stirred overnight. Then, the reaction mixture was extracted with ethyl acetate, washed with brine, and then dried over sodium sulfate. The crude product was purified by silica gel column chromatography (DCM:MeOH=50:1) to give compound 8 was obtained as a white solid (2 g, 50% yield). LC-MS: 350 [M+H]⁺. Step 6-7

To a solution of 8 (3.0 g, 1.0 eq.) in DCE (100 mL) was added a-chloroethyl chloroformate (ACE-Cl, 1,2 eq.) at 0° C. and then refluxing the mixture for 15 h. The intermediate ACE-piperidine formed and is usually deACEylated directly to 9 by evaporating the reaction mixture in vacuo and then heating the residue in MeOH. The residue was dissolved in THF (100 mL), then trimethylamine (3.0 eq.) and Boc₂O (1.3 eq.) was added. The mixture was continued to stir for 3 h at room temperature. The solvent was evaporated under reduced pressure, then water was added, and the mixture was extracted with EA. The combined organic phases were dried over Na₂SO₄ and evaporated. The residue was purified by flash chromatograph to afford 10 (1.5 g, 2 steps, yield 50%). LC-MS: 360 [M+H]⁺, ¹H NMR (600 MHZ, Chloroform-d) 8 7.47 (d, J=7.6 Hz, 1H), 7.08 (d, J=7.6 Hz, 1H), 5.24 (s, 2H), 4.16 (t, J=6.7 Hz, 2H), 3.88 (m, 2H), 3.51 (m, 2H), 2.52 (t. J=6.8 Hz, 2H), 2.13 (m, 2H), 1.61 (m, 2H), 1.46 (s, 9H).

### Step 8

To a solution of compound 10 (2 g, 1 eq.) in tetrahydrofuran (10 mL) and water (10 mL) was added sodium hydroxide (1,2 g, 5 eq.). The mixture was stirred at 20° C. for 16 h. TLC (ethyl acetate: hexane=1:1) showed reaction was complete. The mixture was adjusted to pH=5-6 with aq, hydrochloric acid (1 M) and extracted with ethyl acetate. The organic layer was washed with brine and dried over sodium sulfate. The crude product 11 was not further purificd and used as crude for the next step.

### Step 9

To a solution of compound 11 (2 g, crude, 1 eq.) in dichloromethane (30 mL) was added manganese dioxide (20 eq.). The mixture was stirred at 20° C. for about 1 h. TLC showed reaction was complete. The mixture was diluted with dichloromethane and filtered through a pad of Celite. The filtrate was concentrated in vacuum. The crude product was purified by silica gel column chromatography (DCM:MeOH=10:1). The desired compound 12 was obtained as yellow solid. (1,2 g, 2 steps, 60%). LC-MS: 376 [M+H]⁺.

### Step 10

To a mixture of compound 12 (532 mg, 1.0 eq.) in methanol (5 mL) and dichloromethane (5 mL) was added 3-aminopiperidine-2,6-dione (698 mg, 3 eq. HCl salt), AcONa (698 mg, 6.0 eq.) and AcOH (0.85 mL, 10.0 eq.). The mixture was stirred at 25° C. for 1 h, then sodium cyanoborohydride (268 mg, 3.0 eq.) was added and the mixture was further stirred for 30 min. LCMS showed the reaction was complete. Next, the reaction mixture was quenched with water and concentrated under reduced pressure to give a residue which was purified by pre-HPLC(20%˜50% ACN, neutral). The desired product 13 as a solid (415 mg, yield=60%) after lyophilization.

LC-MS: 488 [M+H]⁺.

### Step 11

To a solution of compound 13 (300 mg 1.0 equiv) in DMF (5 mL) was added HATU (300 mg, 1.3 equiv) and DIPEA (0.35 mL, 3.0 equiv), and the reaction was stirred at rt for 30 min. UPLC-MS indicated a new main peak with desired MS formed, then quenched with water and the mixture was extracted with ethyl acetate, washed with brine, and then dried over sodium sulfate. Compound 14 was obtained as a brown solid (230 mg, 75% yield). LC-MS: 470 [M+H]⁺.

### Step 12

Compound 14 was treated with TFA in DCM at room temperature to de-protect the N-Boc group to provide the cereblon ligand I-27. LC/MS(ESI) m/z: 370.17. 1H NMR (400 MHZ, CDCl₃) δ 8.03 (s, 1H), 7.45 (d, J=1,2 Hz, 2H), 5.22 (dd, J=13.2, 5.1 Hz, 1H), 4.40 (d, J=16.4 Hz, 1H), 4.31-4.20 (m, 3H), 4.11 (brs, 1H), 3.06-2,78 (m, 4H), 2.37 (qd, J=13.1, 5.0 Hz, 1H), 2.22 (dtd, J=13.1, 5.3, 2.7 Hz, 1H), 2.17-2.04 (m, 3H), 1.60-1.50 (s, 4H).

### Intermediate 28: (S)-3-(7′-oxo-7′,9′-dihydro-2′H,8′H-spiro[piperidine-4,3′-[1,4]dioxino[2,3-e]isoindol]-8′-yl)piperidine-2,6-dione

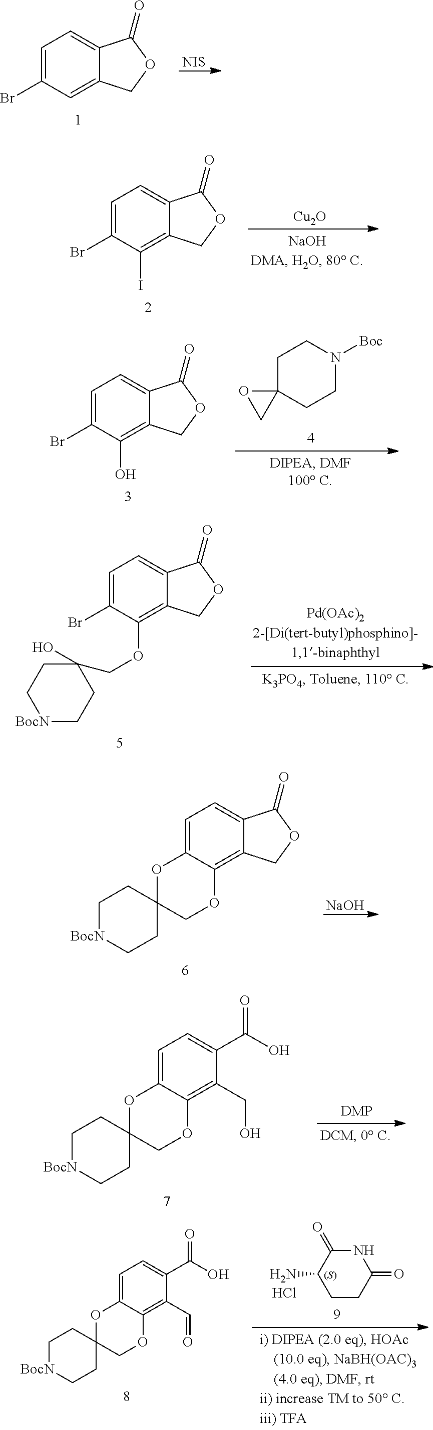

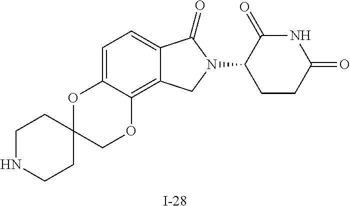

### Step1:5-bromo-4-iodoisobenzofuran-1 (3H)-one

To a solution of 1 (10 g, 1.0 equiv) in CF₃SO₃H (50 mL) was added NIS(1.5 equiv) portionwise at 0° C. The reaction was stirred at rt overnight. Then the reaction mixture was poured into ice-water, and gray solid was precipitated, which is collected by filtration and washed with water. The filter cake was dissolved in DCM, washed with aqeuous Na₂S203, brine, dried over Na₂SO₄ and concentrated to afford a crude product. Further purification by silica gel column chromatography to give the desired product as a white solid 6.55 g.

### Step 2:5-bromo-4-hydroxyisobenzofuran-1 (3H)-one

A mixture of 2 (6.55 g, 1.0 equiv), Cu₂O (553 mg, 0.2 equiv) and NaOH (3.86 g, 5.0 equiv) in DMA/H₂O (40 mL/20 mL) was degassed with N₂ and stirred at 80° C. under N₂ atmosphere overnight. Then the reaction mixture was cooled to rt, neutralized with 2N aq. HCl, extracted with EA, washed with brine, dried over Na₂SO₄, and concentrated to give the crude product, which is purified by silica gel column chromatography to provide compound 3 as a yellow solid 3.67 g (yield=83%), ¹H NMR (400 MHZ, DMSO-d₆) δ 10.89 (s, 1H), 7.72 (d, J=8.0 Hz, 1H), 7.23 (d, J=8.0 Hz, 1H), 5.34 (s, 2H).

### Step3: tert-butyl 4-(((5-bromo-1-oxo-1,3-dihydroisobenzofuran-4-yl)oxy)methyl)-4-hydroxypiperidine-1-carboxylate

To a solution of 3 (500 mg, 1.0 equiv) and 4 (931 mg, 2.0 equiv) in DMF (15 mL) was added DIPEA (3.8 mL, 10.0 equiv), which was stirred at 100° C. for 2 days. Then the reaction mixture was cooled to rt, diluted with EA, washed with brine, dried over Na₂SO₄ and concentrated. The crude product was purified by silica gel column chromatography to give compound 5 as a brown oil 1.03 g, yield>95%. LC-MS: 344.01 [M+H]⁺.

### Step 4: tert-butyl 7′-oxo-7′,9′-dihydro-2′H-spiro[piperidine-4,3′-[1,4]dioxino[2,3-e]isobenzofuran]-1-carboxylate

A mixture of 5 (870 mg, 1.0 equiv), Pd(OAc)₂ (51 mg, 0.2 equiv), 2-[Di (tert-butyl)phosphino]-1,1′-binaphthyl (135 mg, 0.3 equiv) and K₃PO₄ (719 mg, 3.0 equiv) in Toluene (12 mL) was degassed with N₂ and then was stirred at 110° C. under N₂ atmosphere overnight. The reaction mixture was filtered through celite, and the filtration was concentrated under reduced pressure. The result mixture was purified by silica gel column chromatography to give compound 6 as a white solid 540 mg, yield=73%. LC-MS: 362.21 [M+H]⁺.

### Step 5: l′-(tert-butoxycarbonyl)-5-(hydroxymethyl)-3H-spiro[benzo[b][1,4]dioxine-2.4′-piperidine]-6-carboxylic acid

To a solution of 6 (298 mg, 1.0 equiv) in THE/MeOH/H₂O (5 mL/5 mL/3 mL) was added NaOH (330 mg, 10 equiv). The reaction was stirred at rt for 8 h, then concentrated to remove most of the THF/MeOH. The residue was diluted with 4 mL water, followed by neutralization with 2 N aq HCl to PH 4-6, then extracted with DCM. Then combined organic layer was washed with brine, dried with Na₂SO₄, and concentrated to give the desired product 7 as a white solid 284 mg, which was directly used in the next step.

### Step 6: l′-(tert-butoxycarbonyl)-5-formyl-3H-spiro[benzo[b][1,4]dioxine-2.4′-piperidine]-6-carboxylic acid

To a solution of 7 (284 mg, 1.0 equiv) in DCM (15 mL) was added DMP (475 mg, 1.5 equiv) potionwise at 0° C. 5 h Later, the reaction mixture was washed with brine, dried over Na₂SO₄, and concentrated to give the crude product 8, which was directly used in the next step.

### Step 7: (S)-3-(7′-oxo-7′,9′-dihydro-2′H,8′H-spiro[piperidine-4,3′-[1,4]dioxino[2,3-e]isoindol]-8′-yl)piperidine-2,6-dione (1-28)

To a suspension of 9 (46 mg, 2.0 equiv) in DMF (4 mL) was added DIPEA (49 uL, 2.0 equiv), which was stirred at rt for 10 min, followed by addition of AcOH (423 uL, 10.0 equiv), 10 min Later, crude compound 8 (53 mg, 1.0 equiv) was added, and the resulted mixture was stirred at rt for 15 min. Subsequently, NaBH (OAc) 3 (119 mg, 4.0 equiv) was added, and the reaction mixture was stirred overnight. Then the reaction mixture was heated to 50° C. and kept stirring for 12 h. Next, the reaction mixture was concentrated to remove AcOH, and purified by pre-HPLC to give a light purple solid 33 mg. LC-MS: 472.17 [M+H]⁺. Finally, intermediate I-28 was obtained after treatment with TFA. LC-MS: 372.17 [M+H]⁺, ¹H NMR (400 MHZ, Methanol-d₄) δ 7.37 (d, J=8.2 Hz, 1H), 7.13 (d, J=8.2 Hz, 1H), 5.11 (dd, J=13.3, 5.1 Hz, 1H), 4.51-4.35 (m, 2H), 4.17 (d, J=2.0 Hz, 2H), 3.42-3.35 (m, 4H), 2.96-2.84 (m, 1H), 2.82-2,72 (m, 1H), 2.56-2.42 (m, 1H), 2.21-2.06 (m, 3H), 2.01-1.88 (m, 2H), 13C NMR (101 MHZ, MeOD) δ 174.64, 172.26, 171,11, 145.75, 138.95, 131.85, 126.85, 119.67, 118.32, 71.56, 53.74, 46.27, 40.52, 40.49, 32,37, 28.91, 28.85, 24.05.

### Intermediate 29: (S)-3-((S)-3-(hydroxymethyl)-7-oxo-2,3,7,9-tetrahydro-8H-[1,4]dioxino[2,3-e]isoindol-8-yl)piperidine-2,6-dione

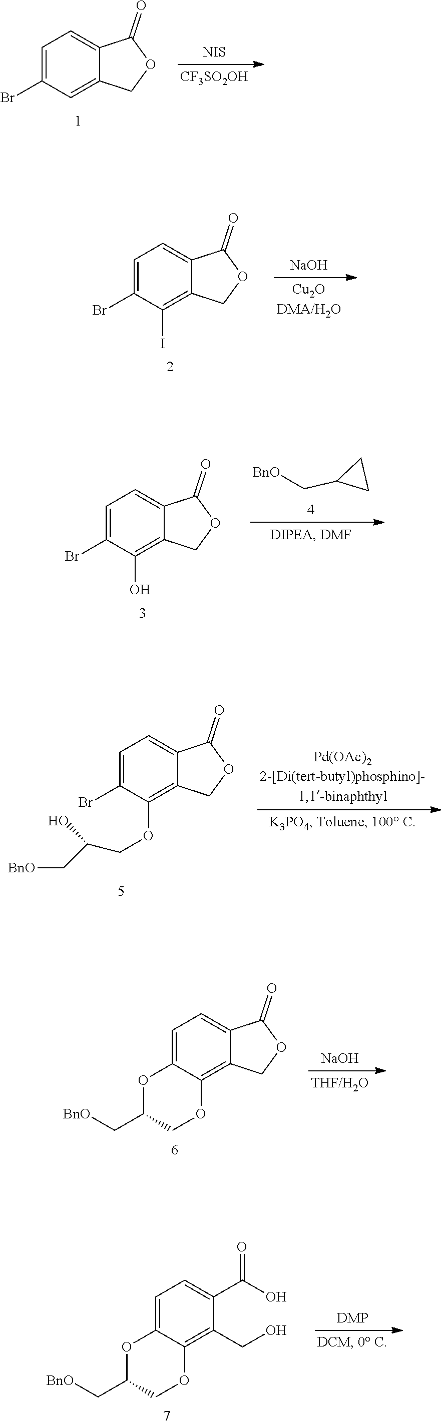

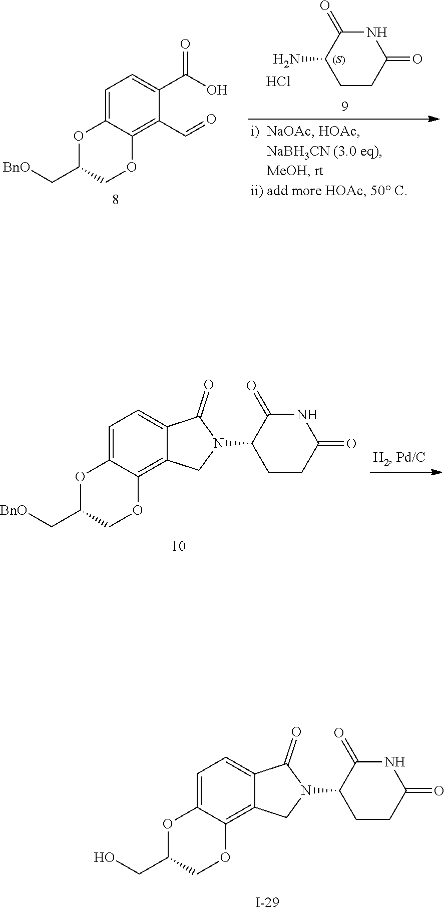

### Step1:5-bromo-4-iodoisobenzofuran-1 (3H)-one

To a solution of 1 (10 g, 1.0 equiv) in CF₃SO₃H (50 mL) was added NIS(1.5 equiv) potionwise at 0° C. The reaction was stirred at rt overnight. Then the reaction mixture was poured into ice-water, and gray solid was precipitated, which is collected by filtration and washed with water. The filter cake was dissolved in DCM, washed with aqeuous Na₂S203, brine, dried over Na₂SO₄ and concentrated to afford a crude product. Further purification by silica gel column chromatography to give the desired product as a white solid 6.55 g.

### Step 2:5-bromo-4-hydroxyisobenzofuran-1 (3H)-one

A mixture of 2 (6.55 g, 1.0 equiv), Cu₂O (553 mg, 0.2 equiv) and NaOH (3.86 g, 5.0 equiv) in DMA/H₂O (40 mL/20 mL) was degassed with N₂ and stirred at 80° C. under N₂ atmosphere overnight. Then the reaction mixture was cooled to rt, neutralized with 2N aq. HCl, extracted with EA, washed with brine, dried over Na₂SO₄, and concentrated to give the crude product, which is purified by silica gel column chromatography to provide compound 3 as a yellow solid 3.67 g (yield=83%), ¹H NMR (400 MHZ, DMSO-d₆) δ 10.89 (s, 1H), 7.72 (d, J=8.0 Hz, 1H), 7.23 (d, J=8.0 Hz, 1H), 5.34 (s, 2H),

### Step3: (S)-4-(3-(benzyloxy)-2-hydroxypropoxy)-5-bromoisobenzofuran-1 (3H)-one

To a solution of 3 (500 mg, 1.0 equiv) and 4 (1015 uL, 3.0 equiv) in DMF (10 mL) was added DIPEA (1.9 mL, 5.0 equiv), which was stirred at 100° C. for 2 days. Then the reaction mixture was cooled to rt, diluted with EA, washed with brine, dried over Na₂SO₄ and concentrated. The crude product was purified by silica gel column chromatography to give compound 5 as a light yellow oil 785 mg, yield=91.5%.

### Step 4: tert-butyl 7′-oxo-7′,9′-dihydro-2′H-spiro[piperidine-4,3′-[1,4]dioxino[2,3-e]isobenzofuran]-1-carboxylate

A mixture of 5 (785 mg, 1.0 equiv), Pd(OAc)₂ (0.2 equiv, 90 mg), 2-[Di (tert-butyl)phosphino]-1,1′-binaphthyl (0.3 equiv, 238 mg) and K₃PO₄ (1270 mg, 3.0 equiv) in Toluene (12 mL) was degassed with N₂ and then was stirred at 100° C. under N₂ atmosphere overnight. The reaction mixture was filtered through celite, and the filtration was concentrated under reduced pressure. The result mixture was purified by silica gel column chromatography to give compound 6 as a light yellow solid 465 mg, yield=74%. LC-MS: 313.01 [M+H]⁺.

### Step 5: (S)-2-((benzyloxy)methyl)-5-(hydroxymethyl)-2,3-dihydrobenzo[b][1,4]dioxine-6-carboxylic acid

To a solution of 6 (465 mg, 1.0 equiv) in THE/MeOH/H₂O (6 mL/6 mL/6 mL) was added NaOH (358 mg, 6.0 equiv). The reaction was stirred at rt overnight, then concentrated to remove most of the THF/MeOH. The residue was diluted with 4 mL water, followed by neutralization with 2 N aq HCl to PH 4-6, then extracted with DCM. Then combined organic layer was washed with brine, dried with Na₂SO₄, and concentrated to give the desired product 7 as a light yellow foam 376 mg, which was directly used in the next step.

### Step 6: (S)-2-((benzyloxy)methyl)-5-formyl-2,3-dihydrobenzo[b][1,4]dioxine-6-carboxylic acid

To a solution of 7 (376 mg, 1.0 equiv) in DCM (15 mL) was added DMP (870 mg, 1.8 equiv) potionwise at 0° C. 1 h Later, the reaction mixture was washed with brine, dried over Na₂SO₄, and concentrated to give the crude product 8, which was directly used in the next step.

### Step 7: (S)-3-((S)-3-((benzyloxy)methyl)-7-oxo-2,3,7,9-tetrahydro-8H-[1,4]dioxino[2,3-e]isoindol-8-yl)piperidine-2,6-dione (1-29)

To a solution of 9 (279 mg, 2.0 equiv) and NaOAc (139 mg, 1.5 equiv) in MeOH (8 mL) was added 8 (370 mg, 1.0 equiv) and AcOH (322 uL, 5.0 equiv), 15 min Later, NaBH₃CN(211 mg, 3.0 equiv) was added in potionwise, and the resulted mixture was stirred at rt for 6 h. Then more AcOH (3.22 mL, 50 equiv) was added, and the reaction mixture was stirred at 50° C. for 3 h. Next, the reaction mixture was concentrated to remove AcOH, and purified by pre-HPLC to give product 10 as a light-yellow oil 222 mg (yield=35%). LC-MS: 423.16 [M+H]⁺. Step 8: (S)-3-((S)-3-(hydroxymethyl)-7-oxo-2,3,7,9-tetrahydro-8H-[1,4]dioxino[2,3-e]isoindol-8-yl)piperidine-2,6-dione

A suspension of 10 (212 mg, 1.0 equiv), Pd/C(10% Pd in C powder, 212 mg) in MeOH was gassed with H₂ and stirred under H₂ atmosphere for 3 h. The reaction mixture was filtered, and the filtration was concentrated to give intermediate I-29 as a white solid 127 mg. LC-MS: 333.11 [M+H]⁺, ¹H NMR (400 MHZ, Methanol-d₄) δ 7.31 (d, J=8.2 Hz, 1H), 7.04 (d, J=8.2 Hz, 1H), 5.10 (dd, J=13.3, 5.2 Hz, 1H), 4.47-4.36 (m, 3H), 4.32-4,24 (m, 1H), 4.21-4.12 (m, 1H), 3.83-3.78 (m, 2H), 2.95-2.82 (m, 1H), 2.81-2,72 (m, 1H), 2.56-2.41 (m, 1H), 2.20-2.10 (m, 1H), 13C NMR (101 MHz, MeOD) δ 174.69, 172.28, 171.35, 148.12, 139.73, 131.67, 126.17, 119.25, 117.68, 75.72, 66.76, 61.72, 53.70, 46.25, 32,39, 24.08.

### Intermediate 30: (S)-3-((R)-3-(hydroxymethyl)-7-oxo-2,3,7,9-tetrahydro-8H-[1,4]dioxino[2,3-e]isoindol-8-yl)piperidine-2,6-dione

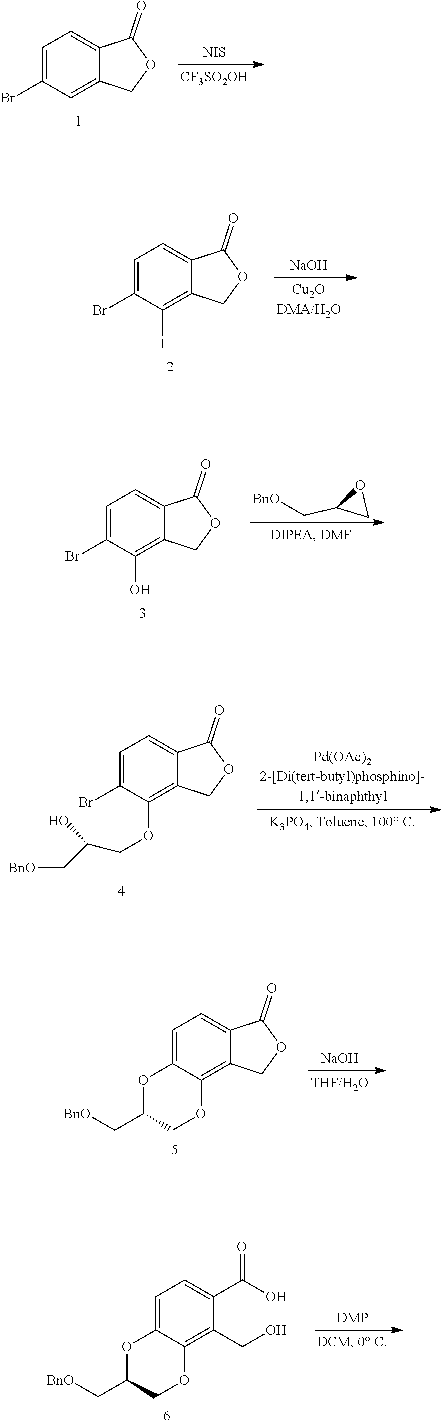

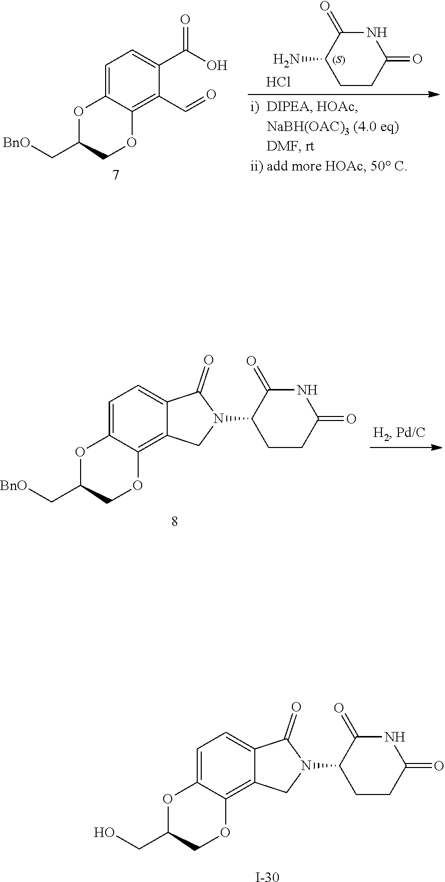

The procedure for making intermediate I-30 is same as that for making intermediate I-29. Intermediate I-30 was obtained as a white solid 193 mg. LC-MS: 333.12 [M+H]⁺, ¹H NMR (400 MHz, Methanol-d₄) δ 7.31 (d, J=8.2 Hz, 1H), 7.04 (d, J=8.2 Hz, 1H), 5.10 (dd, J=13.3, 5.2 Hz, 1H), 4.48-4.32 (m, 3H), 4.32-4,25 (m, 1H), 4.21-4.13 (m, 1H), 3.81 (dd, J=5.1, 1.5 Hz, 2H), 2.95-2.83 (m, 1H), 2.82-2,72 (m, 1H), 2.56-2.42 (m, 1H), 2.20-2.10 (m, 1H),

### Intermediate 31:3-(7′-oxo-7′,9′-dihydro-2′H,8′H-spiro[azetidine-3.3′-[1,4]dioxino[2,3-e]isoindol]-8′-yl)piperidine-2,6-dione

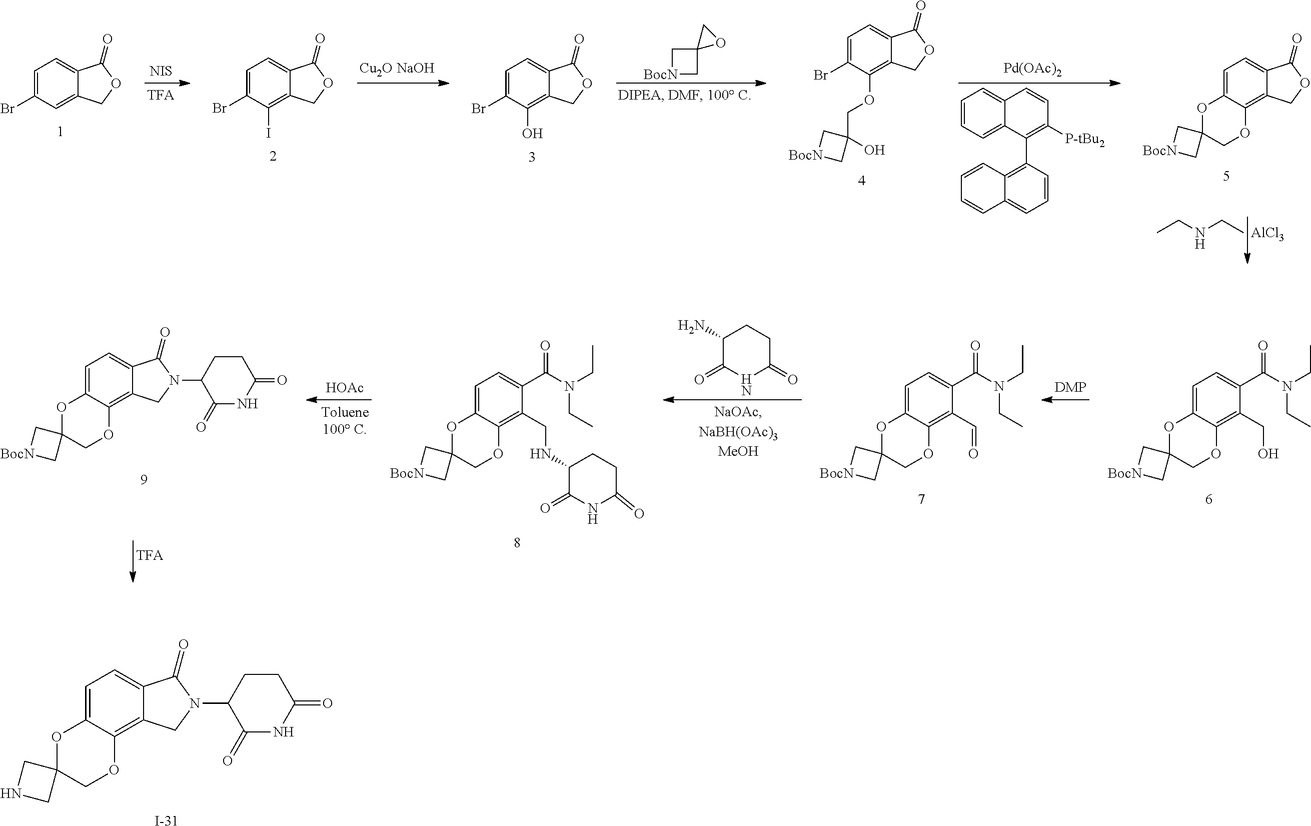

### Step 1 and Step 2 are same as Intermediate 28

### Step3: tert-butyl 3-(((5-bromo-1-oxo-1,3-dihydroisobenzofuran-4-yl)oxy)methyl)-3-hydroxyazetidine-1-carboxylate

To a solution of 3 (1 equiv) in DMF (c1=0.2 mol/L). DIPEA (10 equiv) and epoxide (1.5 equiv) was added into the flask. The reaction was heated to 100° C. The reaction was detected by UPLC-MS. Concentrated directly and purify by silica gel chromatography to give 4 (96% yield).

### Step 4: tert-butyl 7′-oxo-7′,9′-dihydro-2′H-spiro[azetidine-3.3′-[1,4]dioxino[2,3-e]isobenzofuran]-1-carboxylate

To a solution of 4 (1 equiv) in toluene (c1=0.1 mol/L). Pd(OAc)₂ (0.1 equiv), Ligand (0.11 equiv) and K₃PO₄ (3 equiv) was added into the flask under N₂. The reaction was heated to 140° C. under N₂ for 4h. TLC showed reaction was completed. Quenched with saturated NaHCO₃, The organic phase was separated. EA was added to the mixture, the resulting mixture was washed by brine. The conbined organic phase was dried by MgSO₄. Filtered and concentrated in vacuum. The residue was purified by silica gel chromatography (90% yield).

### Step 5: tert-butyl 6′-(diethylcarbamoyl)-5′-(hydroxymethyl)-3′H-spiro[azetidine-3,2′-benzo[b][1,4]dioxine]-1-carboxylate

To a suspension of aluminium trichloride (1.3 equiv.) in DCM (CAICI3=0.5 mol/L), diethylamine (2.5 equiv.) was added at 0° C. and the mixture was stirred for additional 30 min. A solution of 5 (1.0 eq.) in DCM (c10=1 mol/L), was added and the resulting mixture was stirred at 25° C. for 1 h. The reaction mixture was poured into 300 mL saturated aqueous NH₄Cl. The organic layers were combined and washed with 200 mL saturated aqueous NH₄Cl, dried over anhydrous MgSO₄, and concentrated in vacuum. The obtained residue was purified by silica gel chromatography to give 6 (85% yield).

### Step 6: tert-butyl 6′-(diethylcarbamoyl)-5′-formyl-3′H-spiro[azetidine-3,2′-benzo[b][1,4]dioxine]-1-carboxylate

To a solution of 6 (1 equiv) in DCM (c1=0.1 mol/L). DMP (1,1 equiv) was added into the flask at room temperature. Reaction was detected by UPLM-MS. Quenched with saturated NaHCO₃. EA was added to the mixture. The organic phase was separated, the resulting mixture was washed by brine. The combined organic phase was dried by MgSO₄. Filtered and concentrated in vacuum. The residue was purified by silica gel chromatography to give 7 (90%).

### Step 7 to Step 9:3-(7′-oxo-7′,9′-dihydro-2′H,8′H-spiro[azetidine-3.3′-[1,4]dioxino[2,3-e]isoindol]-8′-yl)piperidine-2,6-dione

To a solution of 7 (1 equiv) in MeOH (c1=0.2 mol/L). NaOAc (1.0 equiv) and(S)-3-Amino-piperidine-2,6-dione hydrochloride NaOAc (1.0 equiv), NaCNBH₃ (1.0 equiv) was added into the flask at room temperature. Reaction was detected by UPLM-MS(about 3 hours). Remove solvent under vacuum. The residue was dissolved in toluene. HOAc (15 equiv) was added into flask. The rection was heated at 110° C. and stirred for 12 hour, 9 was purified by HPLC(TFA condition), 1.0 equiv TFA was added and concentrated 9 to get de-Boc I-31 (70% yield in three steps).

### Intermediate 32: (R)-3-(3-((benzyloxy)methyl)-7-oxo-3,4,7,9-tetrahydro-313-[1,4]oxazino[6,5-e]isoindol-8 (2H)-yl)piperidine-2,6-dione

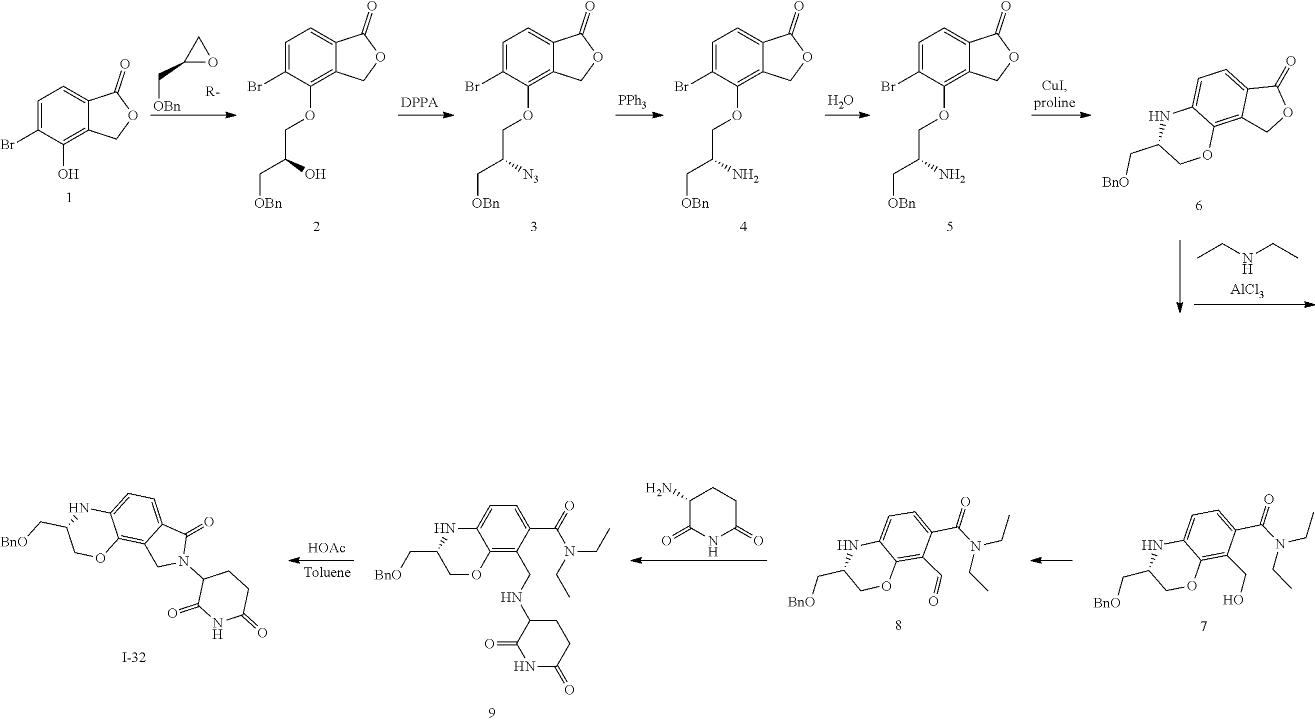

### Step1: (S)-4-(3-(benzyloxy)-2-hydroxypropoxy)-5-bromoisobenzofuran-1 (3H)-one

To a solution of 1 (1 equiv) in DMF (c1=0.2 mol/L). DIPEA (10 equiv) and epoxide (1.5 equiv) was added into the flask. The reaction was heated to 100° C. The reaction was detected by UPLC-MS. Concentrated directly and purify by silica gel chromatography to give 2 (89% yield).

### Step 2: (R)-4-(2-azido-3-(benzyloxy) propoxy)-5-bromoisobenzofuran-1 (3H)-one

To a solution of 2 (1 equiv) in THF (c2=0.1 mol/L). PPh₃ (2 equiv) and DPPA (2 equiv) was added into the flask. Then dropped DIAD (2 equiv). The reaction was quenched with saturated NaHCO₃. EA was added to the mixture, the organic phase was separated. The resulting mixture was washed by brine. The combined organic phase was dried by MgSO₄. Filtered and concentrated in vacuum. The residue was purified by silica gel chromatography to give 7 (80%) Step 3 to 4: (R)-4-(2-amino-3-(benzyloxy) propoxy)-5-bromoisobenzofuran-1 (3H)-one

To a solution of 3 (1 equiv.) in THF (c3=0.2 mol/L). PPh₃ (2 equiv.) was added into the flask. The reaction was heated to 80° C. for 8 h. Then added H₂O (20 equiv.) and heated for 24h. Concentrated directly and purify by silica gel chromatography to give 4 (78% yield).

### Step 5: (S)-3-((benzyloxy)methyl)-3,4-dihydro-2H- isobenzofuro[4,5-b][1,4]oxazin-7 (9H)-one

To a solution of 5 (1 equiv.) in DMF (c5=0.2 mol/L). Cul (0.1 equiv.) and proline (0.1 equiv.) was added into the flask. The reaction was heated under H₂ at 100° C. for 3 h. The reaction was quenched with saturated NaHCO₃. EA was added to the mixture, the organic phase was separated. The resulting mixture was washed by brine. The combined organic phase was dried by MgSO₄. Filtered and concentrated in vacuum. The residue was purified by silica gel chromatography to give 6 (45%)

### Step 6 to 10 are same as 1-31

### Intermediate 33

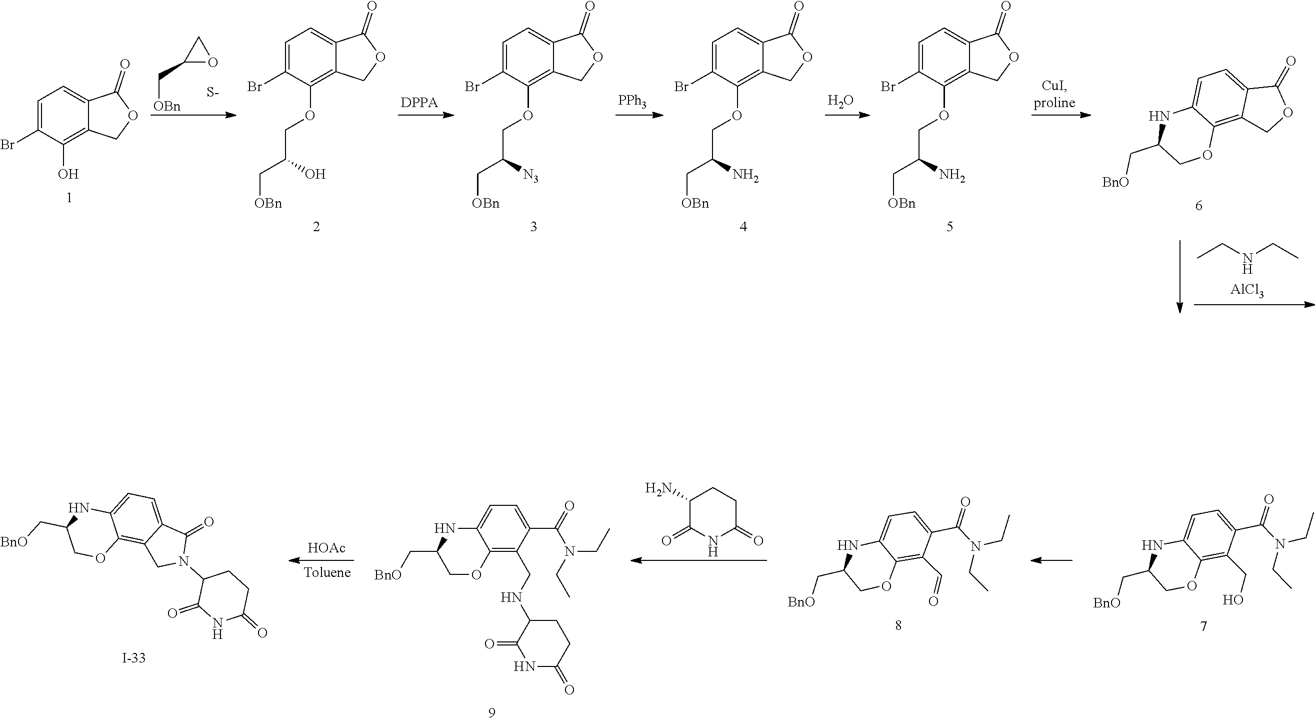

The procedure for making intermediate I-33 is same as that for making intermediate I-32.

### Intermediate 34. (S)-3-((S)-2-((benzyloxy)methyl)-6-oxo-2,3,6,8-tetrahydro-7H-[1,4]dioxino[2,3-f]isoindol-7-yl)piperidine-2,6-dione Intermediate 35: (2S)-2-((benzyloxy)methyl)-7-(2,6-dioxopiperidin-3-yl)-2,3-dihydro-6H-[1,4]dioxino[2,3-f]isoindole-6,8 (7H)-dione

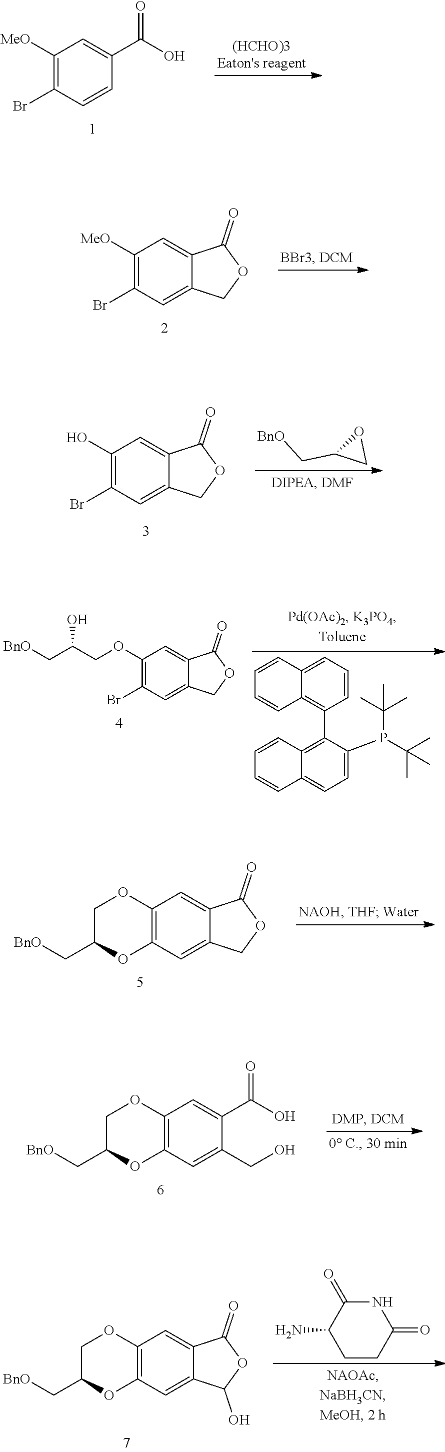

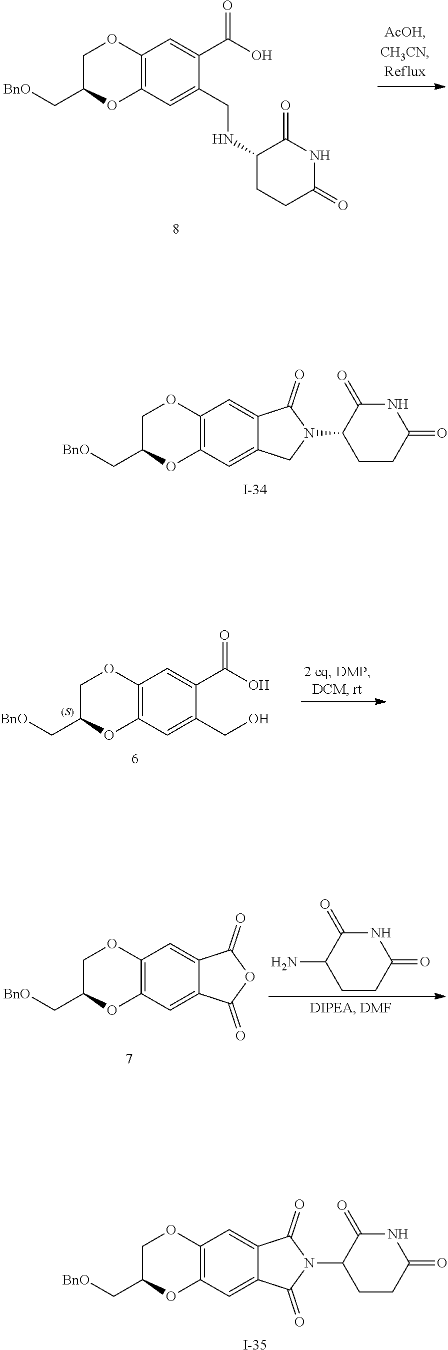

### Step 1:5-bromo-6-methoxyisobenzofuran-1 (3H)-one

To a 100 mL round-bottom flask, Eaton's reagent (30 mL), compound 4-bromo-3-methoxybenzoic acid (1, 5 gm, 21.83 mmol) and Paraformaldehyde (1.96 g, 65 mmol) were added in an ice bath. The resulting mixture was stirred and heated to 50° C. for overnight. After being cooled to room temperature, the reaction mixture was poured into ice-cold water (100 mL) and extracted with dichloromethane (3×60 mL). The combined organic layer was washed with water, saturated NaHCO₃ and brine, dried over anhydrous Na₂SO₄ and concentrated in vacuo, followed purification by silica gel chromatography to give compound 2 in 70% yield.

### Step 2:5-bromo-6-hydroxyisobenzofuran-1 (3H)-one

Over a solution of 5-bromo-6-methoxyisobenzofuran-1 (3H)-one (2 g, 8.29 mmol) in dry CH₂Cl₂ (36 mL) under N₂ atmosphere at −20° C. was added boron tribromide (16.6 mL 1M DCM, 16.6 mmol). Then the solution was stirred at rt for overnight. Next, the reaction was quenched adding a saturated solution of NaHCO₃ (15 mL). The aqueous phase was extracted with CH₂Cl₂ (3×30 mL), and the organic phases were combined, dried over sodium sulfate, filtered and the solvent was removed under reduced pressure. The reaction crude was purified by flash chromatography (20% EtOAc/hexane) affording 3 as a white solid (1.32 g, 70% yield). Step 3: (S)-6-(3-(benzyloxy)-2-hydroxypropoxy)-5-bromoisobenzofuran-1 (3H)-one

To a solution of 3 (1 eq.) in DMF (5 mL/mmol), 10 eq, of DIPEA and 1.5 eq, of epoxide were added into the flask. The reaction was heated to 100° C. The reaction was monitored by UPLC-MS. Concentrated directly and purify by silica gel chromatography to give 4 (96% yield). Step 4: (S)-2-((benzyloxy)methyl)-2,3-dihydro-[1,4]dioxino[2,3-f]isobenzofuran-6 (8H)-one

To a solution of 4 (1 eq.) in toluene (5 mL/mmol). Pd(OAc)₂ (0.1 eq.). [1,1′-binaphthalen]-2-yldi-tert-butylphosphane (0.1 eq.) and K₃PO₄ (3 eq.) was added into the flask under N₂. The reaction was heated to 140° C. under N₂ for 4h. TLC showed reaction was completed. Quenched with saturated NaHCO₃, the organic phase was separated. Ethyl acetate was added to the mixture, the resulting mixture was washed by brine. The combined organic phase was dried by MgSO₄. Filtered and concentrated in vacuum. The residue was purified by silica gel chromatography to give 5 in (70% yield), ¹H NMR (400 MHZ, Chloroform-d) 8 7.45-7.31 (m, 6H), 6.96 (d, J=1.0 Hz, 1H), 5.21 (d, J=0.9 Hz, 2H), 4.63 (d, J=1.8 Hz, 2H), 4.50-4.42 (m, 1H), 4.38 (dd, J=11.6, 2.5 Hz, 1H), 4.22-4.11 (m, 1H), 3.79 (dd, J=10.4, 5.0 Hz, 1H), 3.72 (dd, J=10.4, 5,6 Hz, 1H), Step 5: (S)-2-((benzyloxy)methyl)-7-(hydroxymethyl)-2,3-dihydrobenzo[b][1,4]dioxine-6-carboxylic acid

To a solution of(S)-2-((benzyloxy)methyl)-2,3-dihydro-[1,4]dioxino[2,3-f]isobenzofuran-6 (8H)-one (5, 312 mg, 1 mmol, 1 eq.) in tetrahydrofuran (4 mL) and water (4 mL) was added sodium hydroxide (200 mg, 5 eq.). The mixture was stirred at 20° C. for 16 h. TLC (ethyl acetate: hexane=1:1) showed reaction was complete. The mixture was adjusted to pH=5 with aq, hydrochloric acid (1 M) and extracted with ethyl acetate (10 mL×3). The organic layer was washed with brine (10×2 mL) and dried over sodium sulfate. The crude material (6) was not further purified and used as crude for the next steps. LC/MS (ESI) m/z: 331,12 (M+H)

### Step 6: (2S)-2-((benzyloxy)methyl)-8-hydroxy-2,3-dihydro-[1,4]dioxino[2,3-f]isobenzofuran-6 (8H)-one

To a solution of 6 (1.0 eq . . , 330 mg) in DCM (10 mL) was added DMP (1,2 eq.) at 0° C. and stirred it for 30 mins. TLC (ethyl acetate: hexane=1:1) showed reaction was complete. The reaction mixture was then diluted with DCM, washed with brine, dried over and concentrated under reduced pressure. The residue was purified by silica gel chromatography to give 7 in (70% yield). LC/MS (ESI) m/z: 346.12 (M+H₂O).

### Step 7&8: (S)-3-((S)-2-((benzyloxy)methyl)-6-oxo-2,3,6,8-tetrahydro-7H-[1,4]dioxino[2,3-f]isoindol-7-yl)piperidine-2,6-dione

A mixture of 7 (1.0 eq., 303 mg), (S)-3-aminopiperidine-2,6-dione (1.5 e eq., 199.5 mg) and NaOAc (3.0 eq., 198 mg) was dissolved in methanol: DCM (1:1, 20 mL), and kept stirring at rt for 20 min. Then NaBH₃CN(2.0 eq . . , 124 mg) was added, 2 h Later, UPLC-MS showed the starting material 7 was completely conversion and a new main peak 8 with desired MS formed. LC/MS (ESI) m/z: 441,16 (M+H). The crude compound 8 (1.0 eq., 440 mg) was dissolved in CH₃CN(4 mL) and was treated with HOAc (15 eq.). The rection was heated at 60° C. and stirred for 2h. Next, the reaction mixture was quenched with water and concentrated under reduced pressure to give a residue which was purified by pre-HPLC to give I-34 in (70% yield). LC/MS (ESI) m/z: 423.15 (M+H), ¹H NMR (400 MHZ, Chloroform-d) δ 8.59 (d, J=3.4 Hz, 1H), 7.48-7.30 (m, 6H), 6.97 (s, 1H), 5.21 (dd, J=13.2, 5.2 Hz, 1H), 4.62 (s, 2H), 4.48-4.31 (m, 3H), 4.24 (dd, J=15.9, 5,6 Hz, 1H), 4.14 (ddd, J=11.6, 7.1, 2.0 Hz, 1H), 3.84-3.62 (m, 2H), 2.99-2,72 (m, 2H), 2.30 (td, J=12.7, 5,6 Hz, 1H), 2.20 (dq. J=8.0, 4.1, 3,4 Hz, 1H), 13C NMR (101 MHZ, Chloroform-d) 8 171.65, 169.81, 143.89. 137.44, 135.12, 128.55, 128.01, 127.80, 127.77, 124.14, 112.61, 111.68. 73.76, 73.74, 72.59, 72.55, 68.33, 68.30, 65.33, 52.05, 46.79, 31,47, 23.33. Step 9: (S)-2-((benzyloxy)methyl)-2,3-dihydro-[1,4]dioxino[2,3-f]isobenzofuran-6,8-dione

To a solution of 6 (1.0 eq., 330 mg) in DCM (10 mL) was added DMP (2.0 eq.) at 0° C. and stirred it for 2 h at room temperature. TLC (ethyl acetate: hexane=1:1) showed reaction was complete. The reaction mixture was then diluted with DCM, washed with brine, dried over and concentrated under reduced pressure. The residue was purified by silica gel chromatography to give 9 in (70% yield). LC/MS (ESI) m/z: 327.10 (M+H),

### Step 10: (2S)-2-((benzyloxy)methyl)-7-(2,6-dioxopiperidin-3-yl)-2,3-dihydro-6H-[1,4]dioxino[2,3-f]isoindole-6,8 (7H)-dione

Compound 9 (50 mg, 0.15 mmol) and DIPEA (10 eq.) were dissolved in dry DMF (4 mL), and the reaction mixture was stirred at 60° C. for 2h. TLC showed the reaction was complete. The crude product which was purified by flash chromatography on silica gel (ethyl acetate: hexane=1:1) to give compound I-35 in 80% yield. LC/MS (ESI) m/z: 437.15 (M+H), ¹H NMR (400 MHZ, Chloroform-d): δ 7.97 (s, 1H), 7.46-7.30 (m, 7H), 4.95 (dd, J=12.2, 5.3 Hz, 1H), 4.63 (d, J=2.1 Hz., 2H), 4.51-4.37 (m, 2H), 4.22 (dd, J=12.0, 7.6 Hz, 1H), 3.85-3.68 (m, 2H), 2.98-2.69 (m, 3H), 2.23-2.08 (m, 1H),

### Intermediate 36. (S)-3-((S)-1-oxo-1,3,5,5a,6,7,8,9-octahydro-2H-pyrazino[1′,2′: 4,5][1,4]oxazino[2,3-e]isoindol-2-yl)piperidine-2,6-dione

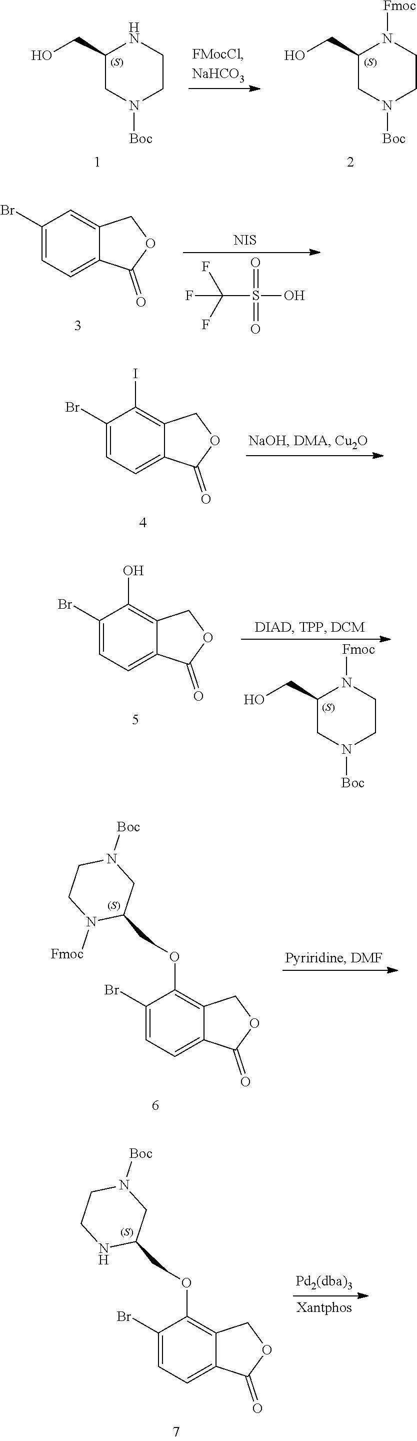

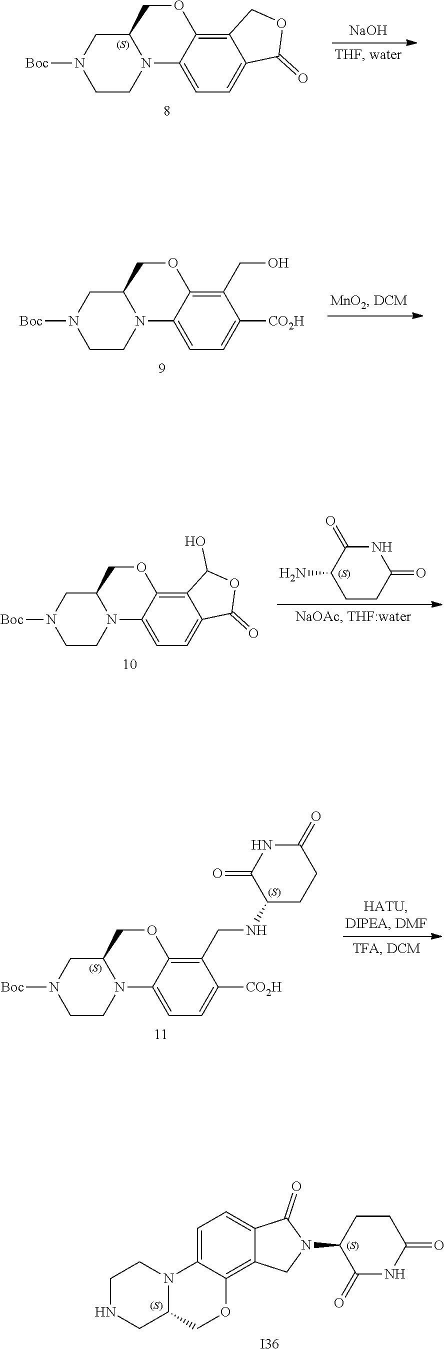

Intermediate I-36 was made using the similar procedure for making intermediate I-23.

¹H NMR of compound I-36 (400 MHZ, Methanol-d₄) δ 7.37 (d, J=8.3 Hz, 1H), 7.14 (d, J=8.5 Hz, 1H), 5.11 (ddd, J=13.3, 5.2, 2.2 Hz, 1H), 4.50-4.34 (m, 3H), 4.34-4.10 (m, 3H), 3.67-3.42 (m, 4H), 3.30-3,22 (m, 1H), 3.22-3.09 (m, 1H), 3.02 (td, J=12.2, 5.7 Hz, 1H), 2.91 (ddd, J=18.5, 13,4, 5.4 Hz, 1H), 2.79 (ddd, J=17.6, 4.7, 2.4 Hz, 1H), 2.57-2.41 (m, 1H), 2.16 (dtd, J=12.9, 5.3, 2.5 Hz, 1H).

### Intermediate 37. (S)-3-((2S,3aS)-2-amino-7-oxo-2,3,3a,4,7,9-hexahydro-1H,8H-pyrrolo[1′,2′: 4,5][1,4]oxazino[2,3-f]isoindol-8-yl)piperidine-2,6-dione

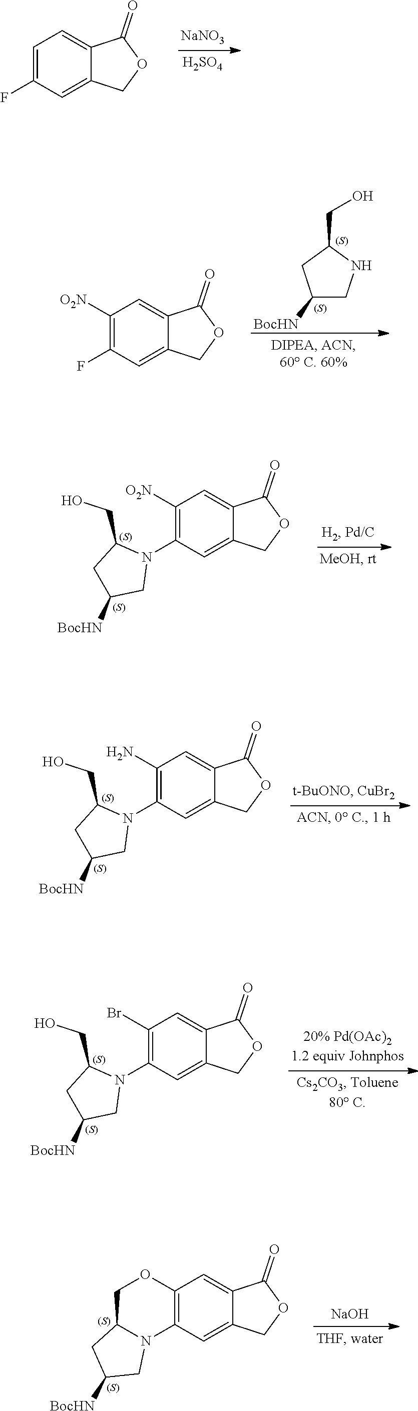

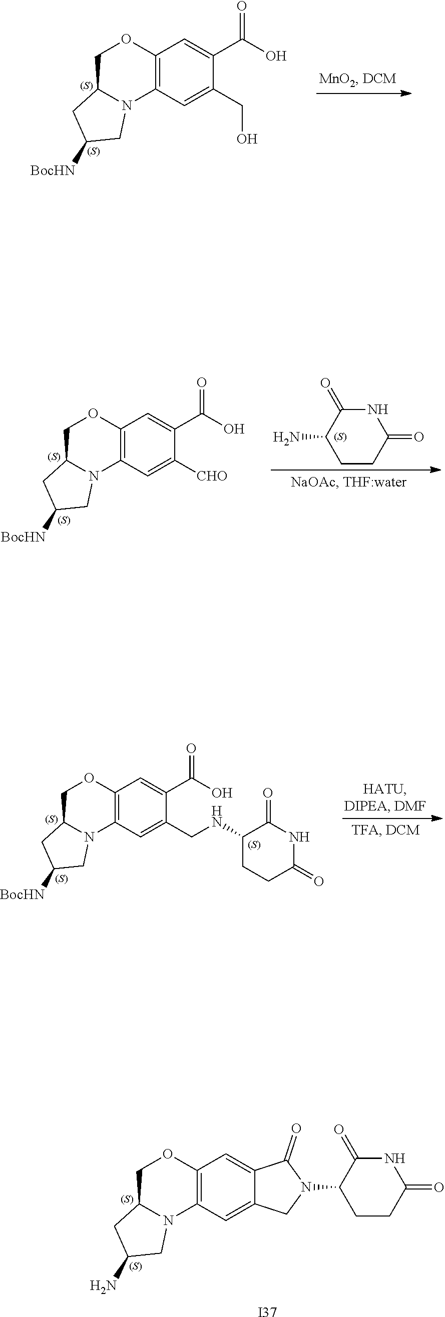

Intermediate I-37 was made using the similar procedure for making intermediate I-24.

¹H NMR of compound I-37 (400 MHZ, Methanol-d₄) d 7.16 (s, 1H), 6.72 (s, 1H), 5.09 (dt, J=13.3, 5.1 Hz, 1H), 4.58 (d, J=7.1 Hz, 2H), 4.36 (d, J=6.7 Hz, 2H), 4.15 (d, J=3.6 Hz, 1H), 3.79 (dd, J=10.4, 7.9 Hz, 1H), 3.72-3.62 (m, 2H), 3.53-3.40 (m, 1H), 2.96-2.84 (m, 1H), 2.78 (ddd, J=17.4, 4.8, 2.5 Hz, 1H), 2.61 (ddd, J=12.5, 8.6, 4.1 Hz, 1H), 2.55-2.37 (m, 1H), 2.16 (ddq, J=10.4, 5.3, 2.7 Hz, 1H), 1.79-1.59 (m, 1H),

### Intermediate 38. (R)-3-((S)-3-oxo-1,3,7,7a,8,9,10,11-octahydro-2H-pyrazino[1′,2′: 4,5][1,4]oxazino[3,2-e]isoindol-2-yl)piperidine-2,6-dione

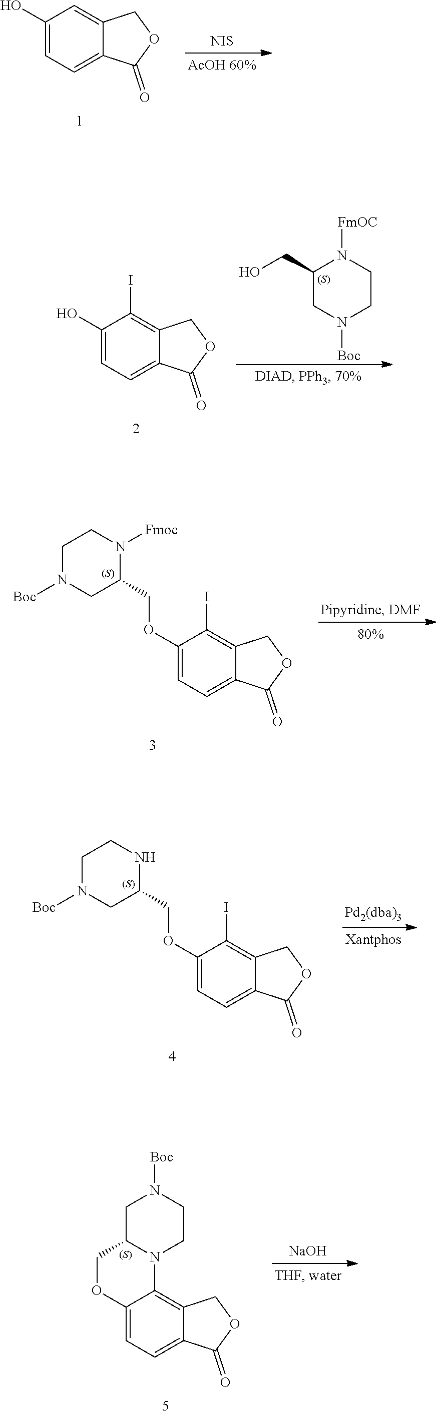

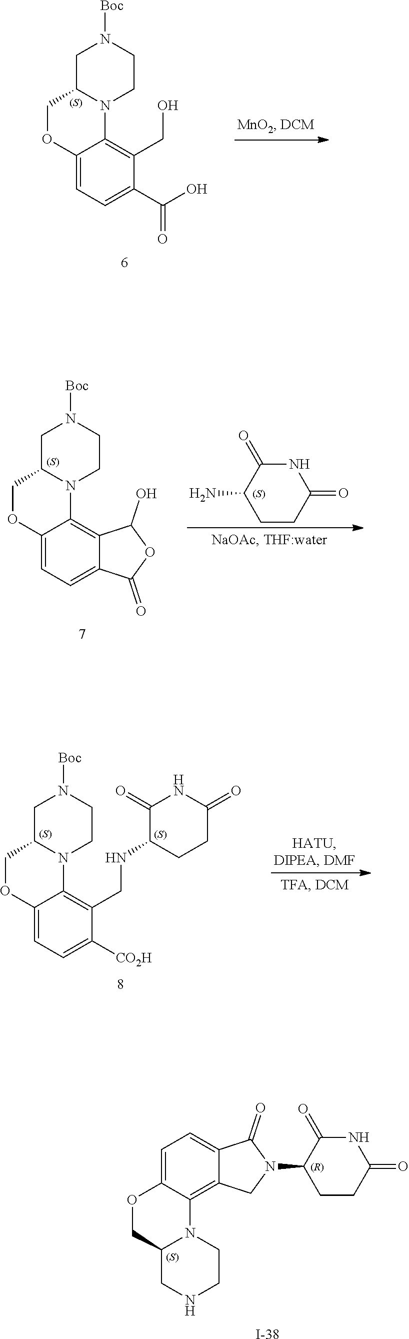

Intermediate I-38 was made using the similar procedure for making intermediate I-23.

¹H NMR of compound I-38 (400 MHZ, Methanol-d₄) δ 7.32 (d, J=8.1 Hz, 1H), 7.02 (dd, J=8.1, 0.9 Hz, 1H), 5.15 (dd, J=13,4, 5.2 Hz, 1H), 4.70-4.47 (m, 2H), 4.34 (ddd, J=11.3, 4.2, 2.8 Hz, 1H), 4.14 (ddd, J=11.3, 9.8, 7.2 Hz, 1H), 4.04-3.91 (m, 1H), 3.65 (ddq, J=10.4, 7.1, 3,4, 2.8 Hz, 1H), 3.54-3.39 (m, 2H), 3.30-3,22 (m, 2H), 3.14 (dt, J=12.8, 10.6 Hz, 1H), 2.94 (ddd, J=17.6, 13.5, 5.4 Hz, 1H), 2.80 (ddd, J=17.6, 4.7, 2.4 Hz, 1H), 2.60-2.42 (m, 1H), 2.19 (ddq, J=10.5, 5.4, 2.8 Hz, 1H),

### Intermediates 39 and 40: (5S,6S)-5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol & (5R,6R)-5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol

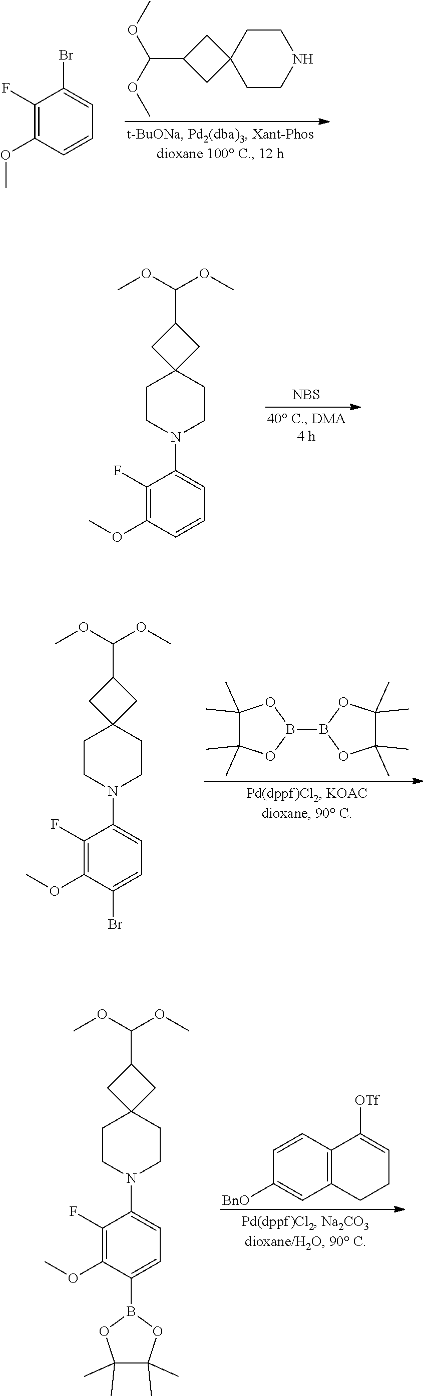

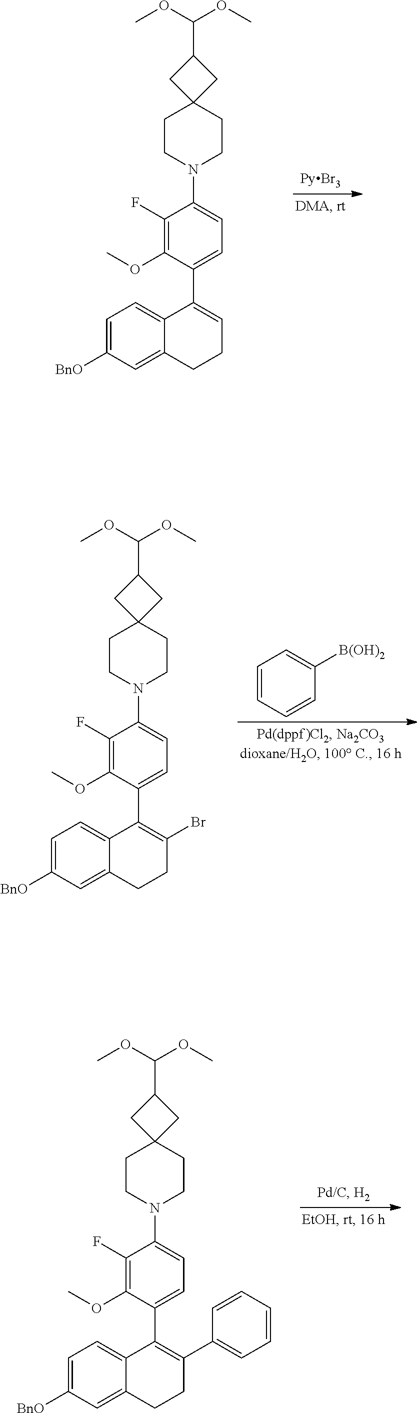 [SMILES: COc1c(C2=CCCc3cc(OCc4ccccc4)ccc32)ccc(N2CCC3(CC2)CC(C(OC)OC)C3)c1F ; COc1c(C2=C(Br)CCc3cc(OCc4ccccc4)ccc32)ccc(N2CCC3(CC2)CC(C(OC)OC)C3)c1F ; OB(O)c1ccccc1 ; COc1c(C2=C(c3ccccc3)CCc3cc(OCc4ccccc4)ccc32)ccc(N2CCC3(CC2)CC(C(OC)OC)C3)c1F]

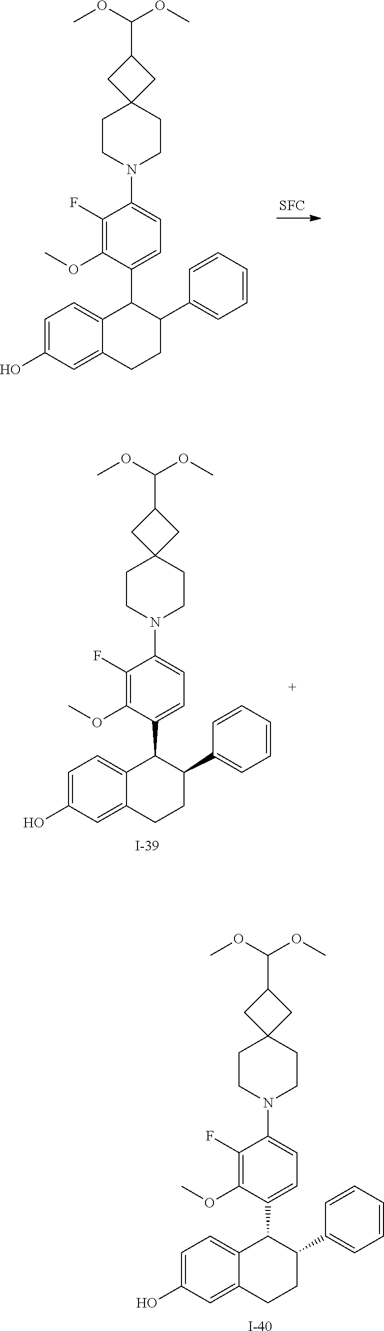

### Step 1:2-(dimethoxymethyl)-7-(2-fluoro-3-methoxyphenyl)-7-azaspiro[3.5]nonane

A mixture of 1-bromo-2-fluoro-3-methoxybenzene (7.00 g, 1 eq, 34.1 mmol), Pd₂(dba) 3 (1.56 g, 0.05 eq, 1.71 mmol), t-BuONa (3.28 g, 1 eq, 34.1 mmol), 2-(dimethoxymethyl)-7-azaspiro[3.5]nonane (7.48 g, 1,1 eq, 37.6 mmol), xantphos (1.98 g, 0.1 eq, 3.41 mmol) in 1,4-Dioxane (70.0 mL) was purged with nitrogen and heated to 100° C. for 16h. TLC showed the reaction was complete. The mixture was diluted with ethyl acetate (50 mL) and washed with water (50 mL). The organic layer was washed with brine (50 mL), dried over anhydrous Na₂SO₄, filtered and evaporated to dryness. The crude product was purified by silica gel column chromatography using 0-50% EtOAc/hexane to give 2-(dimethoxymethyl)-7-(2-fluoro-3-methoxyphenyl)-7-azaspiro[3.5]nonane (2.20 g, 19.9%), LC-MS (ESI, m/z): mass calcd. For C18H₂₆FNO₃, 323,4; found, 324.1 [M+H]⁺.

### Step 2:7-(4-bromo-2-fluoro-3-methoxyphenyl)-2-(dimethoxymethyl)-7-azaspiro[3.5]nonane

To a mixture of 2-(dimethoxymethyl)-7-(2-fluoro-3-methoxyphenyl)-7-azaspiro[3.5]nonane (2.80 g, 1 eq, 8.66 mmol) in DMA (30.0 mL) was added NBS (1,46 g, 0.95 eq, 8.22 mmol) slowly at 0° C. over 30 minutes. The mixture was stirred at 0° C. for 1 hour. LC-MS showed the reaction was completed. The reaction was added with 10 mL of Sat. NH₄Cl solution, followed by 110 mL of water, then extracted with EtOAc (120 mL×3). The combined organic layers were washed with water (120 mL×2) and brine (120 mL) successively, then dried over anhydrous Na₂SO₄, filtered and evaporated to dryness. The residue was purified by silica gel column chromatography using 0-50% EtOAc/hexane to afford 7-(4-bromo-2-fluoro-3-methoxyphenyl)-2-(dimethoxymethyl)-7-azaspiro[3.5]nonane (2.70 g, 77.5%) as a yellow solid. LC-MS (ESI, m/z): mass calcd. For C18H₂₅BrFNO₃, 401.30; found, 402.0 [M+H]⁺.

### Step 3:2-(dimethoxymethyl)-7-(2-fluoro-3-methoxy-4-(4,4,5,5-tetramethyl-1,3,2-dioxaborolan-2-yl)phenyl)-7-azaspiro[3.5]nonane

To a mixture of 7-(4-bromo-2-fluoro-3-methoxyphenyl)-2-(dimethoxymethyl)-7-azaspiro[3.5]nonane (1.00 g, 1 eq, 2.49 mmol), Bis (pinacolato)diboron (1,26 g, 2 eq, 4.97 mmol) in 1,4-Dioxane (10.0 mL) was added PdCl₂(dppf) (182 mg, 0.1 eq, 249 μmol) and KOAC(732 mg, 3 eq, 7.46 mmol). The mixture was stirred at 90° C. for 16 hours under Ar, then cooled to rt and diluted with ethyl acetate (100 mL) and washed with water (50 mL). The organic phase was washed with brine (50 mL), dried over Na₂SO₄ and filtered. The filtrate was evaporated in vacuo and the residue was purified by column chromatography on silica gel eluted with 0-50% EtOAc/hexane to afford 2-(dimethoxymethyl)-7-(2-fluoro-3-methoxy-4-(4,4,5,5-tetramethyl-1,3,2-dioxaborolan-2-yl)phenyl)-7-azaspiro[3.5]nonane (600 mg, 53.7%) as a white solid. LC-MS (ESI, m/z): mass calcd. For C₂₄H₃₇BFNO₅, 449.3; found, 450.4 [M+H]⁺.

### Step 4:7-(4-(6-(benzyloxy)-3,4-dihydronaphthalen-1-yl)-2-fluoro-3-methoxyphenyl)-2-(dimethoxymethyl)-7-azaspiro[3.5]nonane

To a mixture of 2-(dimethoxymethyl)-7-(2-fluoro-3-methoxy-4-(4,4,5,5-tetramethyl-1,3,2-dioxaborolan-2-yl)phenyl)-7-azaspiro[3.5]nonane (200 mg, 1 eq, 445 μmol), 6-(benzyloxy)-3,4-dihydronaphthalen-1-yl trifluoromethanesulfonate (188 mg, 1,1 eq, 490 μmol). Na₂CO₃ (94.3 mg, 2 eq, 890 μmol) in 1,4-Dioxane (10.0 mL) and H₂O (1.00 mL) was added PdCl₂(dppf) (32.6 mg, 0.1 eq, 44.5 μmol) and the mixture was stirred at 90° C. for 16 hours under Ar. The mixture was diluted with ethyl acetate (100 mL) and washed with water (50 mL). The organic phase was washed with brine (50 mL), then dried over Na₂SO₄ and filtered. The filtrate was evaporated in vacuo and the residue was purified by column chromatography on silica gel eluted with 0-50% EtOAc/hexane to afford 7-(4-(6-(benzyloxy)-3,4-dihydronaphthalen-1-yl)-2-fluoro-3-methoxyphenyl)-2-(dimethoxymethyl)-7-azaspiro[3.5]nonane (200 mg, 80.6%) as a white solid. LC-MS (ESI, m/z): mass calcd. For C₃₅H₄₀FNO₄, 557.7; found, 558.4 [M+H]⁺.

### Step 5:7-(4-(6-(benzyloxy)-2-bromo-3,4-dihydronaphthalen-1-yl)-2-fluoro-3-methoxyphenyl)-2-(dimethoxymethyl)-7-azaspiro[3.5]nonane

To a mixture of 7-(4-(6-(benzyloxy)-3,4-dihydronaphthalen-1-yl)-2-fluoro-3-methoxyphenyl)-2-(dimethoxymethyl)-7-azaspiro[3.5]nonane (900 mg, 1 eq, 1.61 mmol) in DMA (10.0 mL) was added PyBr₃ (515 mg, 1 eq, 1.61 mmol) slowly at 0° C. over 30 minutes. The mixture was stirred at 0° C. for 1 hour. LC-MS showed the reaction was completed, 10 mL of Sat. NH₄Cl solution was added, followed by 110 mL of water, and the resulting mixture was extracted with EtOAc (120 mL×3). The combined organic layers were washed with water (120 mL×2) and brine (120 mL) successively, then dried over Na₂SO₄, filtered. The filtrate was evaporated to afford 7-(4-(6-(benzyloxy)-2-bromo-3,4-dihydronaphthalen-1-yl)-2-fluoro-3-methoxyphenyl)-2-(dimethoxymethyl)-7-azaspiro[3.5]nonane (1.00 g, 1.57 mmol, 97.3%) as a yellow solid. LC-MS (ESI, m/z): mass calcd. For C₃₅H₃₉BrFNO₄, 635.3; found, 636.4 [M+H]⁺.

### Step 6:7-(4-(6-(benzyloxy)-2-phenyl-3,4-dihydronaphthalen-1-yl)-2-fluoro-3-methoxyphenyl)-2-(dimethoxymethyl)-7-azaspiro[3.5]nonane

A mixture of 7-(4-(6-(benzyloxy)-2-bromo-3,4-dihydronaphthalen-1-yl)-2-fluoro-3-methoxyphenyl)-2-(dimethoxymethyl)-7-azaspiro[3.5]nonane (1.00 g, 1 eq, 1.57 mmol), phenylboronic acid (383 mg, 2 eq, 3.14 mmol), Na₂CO₃ (333 mg, 2 eq, 3.14 mmol), PdCl₂(dppf) (115 mg, 0.1 eq, 157 μmol) in 1,4-dioxane (10.0 mL) and H₂O (2.00 mL) was stirred at 100° C. for 16 hours under Ar. The mixture was diluted with ethyl acetate (100 mL) and washed with water (50 mL). The organic phase was washed with brine (50 mL), then dried over Na₂SO₄ and filtered. The filtrate was evaporated in vacuo and the residue was purified by column chromatography on silica gel eluted with 0-50% EtOAc/hexane to afford 7-(4-(6-(benzyloxy)-2-phenyl-3,4-dihydronaphthalen-1-yl)-2-fluoro-3-methoxyphenyl)-2-(dimethoxymethyl)-7-azaspiro[3.5]nonane (500 mg, 50.2%) as a yellow solid. LC-MS (ESI, m/z): mass calcd. For C₄₁H₄₄FNO₄, 633.8; found, 634,4 [M+H]⁺.

### Step 7:5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol

A mixture of 7-(4-(6-(benzyloxy)-2-phenyl-3,4-dihydronaphthalen-1-yl)-2-fluoro-3-methoxyphenyl)-2-(dimethoxymethyl)-7-azaspiro[3.5]nonane (400 mg, 1 eq, 631 μmol), Pd/C(67 mg, 10% on Carbon, wetted with ca, 55% water) in MeOH (10.0 mL) was heated to 40° C. for 16 hours under H₂ atmosphere (1 atm). LC-MS showed the reaction was completed. The reaction mixture was cooled to room temperature, filtered. The filter cake was washed with EtOAc (20 mL×2). The filtrate was evaporated and the residue purified by column chromatography on silica gel eluted with 0-50% EtOAc/hexane to to afford 5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol (200 mg, 367 μmol, 58.1%) as a yellow solid. LC-MS (ESI, m/z): mass calcd. For C₃₄H₄₀FNO₄, 545.29; found, 546.4 [M+H]⁺.

### Step 8: (5S,6S)-5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol & (5R,6R)-5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol

The tert-butyl 5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol (200 mg) was separated by SFC to afford compound (5S,6S)-5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol (80 mg) as a white solid and (5R,6R)-5-(4-(2-(dimethoxymethyl)-7-azaspiro[3.5]nonan-7-yl)-3-fluoro-2-methoxyphenyl)-6-phenyl-5,6,7,8-tetrahydronaphthalen-2-ol (80 mg) as a white solid. LC-MS (ESI, m/z): mass calcd. For C₃₄H₄₀FNO₄, 545.29; found, 546.4 [M+H]⁺.

### Intermediate 41:4-(2,2-dimethoxyethyl)piperidine

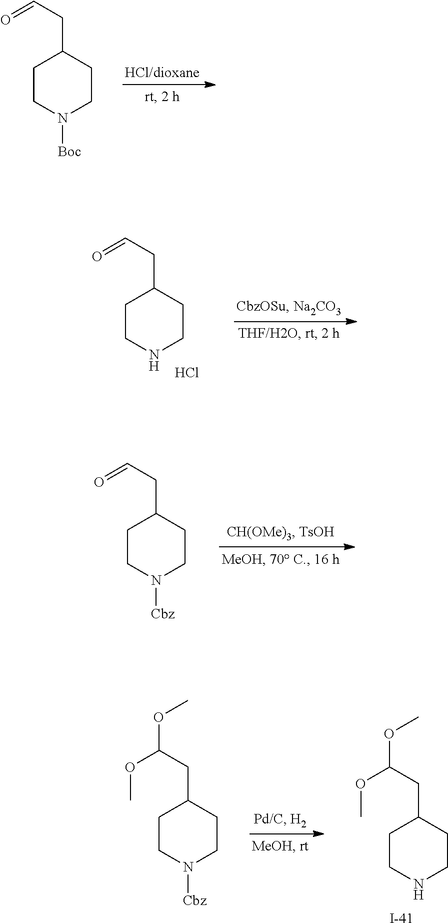

### Step 1:2-(piperidin-4-yl)acetaldehyde hydrochloride

A solution of tert-butyl 4-formylpiperidine-1-carboxylate (3.00 g, 13.2 mmol, 1 eq) in HCl/dioxane (15 mL) was stirred at room temperature for 2 h. The mixture was concentrated to afford 2-(piperidin-4-yl)acetaldehyde hydrochloride (2.4 g, crude) as a yellow solid. LC-MS (ESI, m/z): mass calcd. For C₇H₁₃NO, 127.1; found, 128.3 [M+H]⁺.

### Step 2: benzyl 4-(2-oxoethyl)piperidine-1-carboxylate

To a mixture of 2-(piperidin-4-yl)acetaldehyde hydrochloride (2.4 g, 1 eq, 14.7 mmol) in THF (10 mL) and H₂O (5 mL) was added Na₂CO₃ (7.8 g, 5 eq, 73.6 mmol), CbzOSu (4,4 g, 1,2 eq, 17.7 mmol). The mixture was stirred at room temperature for 1 hour. The reaction solution was diluted with H₂O (20 mL), extracted with EtOAc (20 mL×2). The combined organic layer was washed with brine (50 mL), dried over Na₂SO₄, filtered and concentrated in vacuum. The residue was purified by column chromatography on silica gel eluting with (PE:EA=2:1) to give benzyl 4-(2-oxoethyl)piperidine-1-carboxylate (2,30 g, 59.9%) as light yellow oil. LC-MS (ESI, m/z): mass calcd. For C₁₅H₁₉NO₃, 261,1; found, 262.2 [M+H]⁺.

### Step 3: benzyl 4-(2,2-dimethoxyethyl)piperidine-1-carboxylate

To a solution of benzyl 4-(2-oxoethyl)piperidine-1-carboxylate (2.10 g, 1 eq, 8.04 mmol) in MeOH (20 mL) was added CH(OMe)₃ (2.56 g, 3 eq, 24.1 mmol) followed by TsOH.H₂O (138 mg, 0.1 eq, 804 μmol) and the mixture was stirred at 70° C. for 12 h. The mixture was poured into Na₂CO₃ aqueous solution (20 mL), extracted with EtOAc (20 mL×2). The combined organic layer was washed with brine (50 mL), dried over Na₂SO₄, filtered and concentrated in vacuum. The crude product was purified by column chromatography on silica gel eluting with 20% EA in PE to afford benzyl 4-(2,2-dimethoxyethyl)piperidine-1-carboxylate (960 mg, 38.9%) as light yellow oil. LC-MS (ESI, m/z): mass calcd. For C₁₇H₂₅NO₄, 307.2; found, 308.2 [M+H]⁺.

### Step 4:4-(2,2-dimethoxyethyl)piperidine

To a solution of compound benzyl 4-(2,2-dimethoxyethyl)piperidine-1-carboxylate (6.30 g, 1 eq, 20.5 mmol) in MeOH (30 mL) was added Pd/C(1.97 g, 10% on Carbon, wetted with ca. 55% water) and the mixture was stirred at room temperature for 16 h under H₂. The mixture was filtered and filtrate was concentrated to give 4-(2,2-dimethoxyethyl)piperidine (3.30 g, 92.9%) as a white paste. LC-MS (ESI, m/z): mass calcd. For C₉H₁₉NO₂, 173.1; found, 174.3 [M+H]⁺.

### Intermediate 42:2-(1-(2-fluoro-4-((1S,2S)-6-hydroxy-2-phenyl-1,2,3,4-tetrahydronaphthalen-1-yl)-5-methoxyphenyl)piperidin-4-yl)acetaldehyde

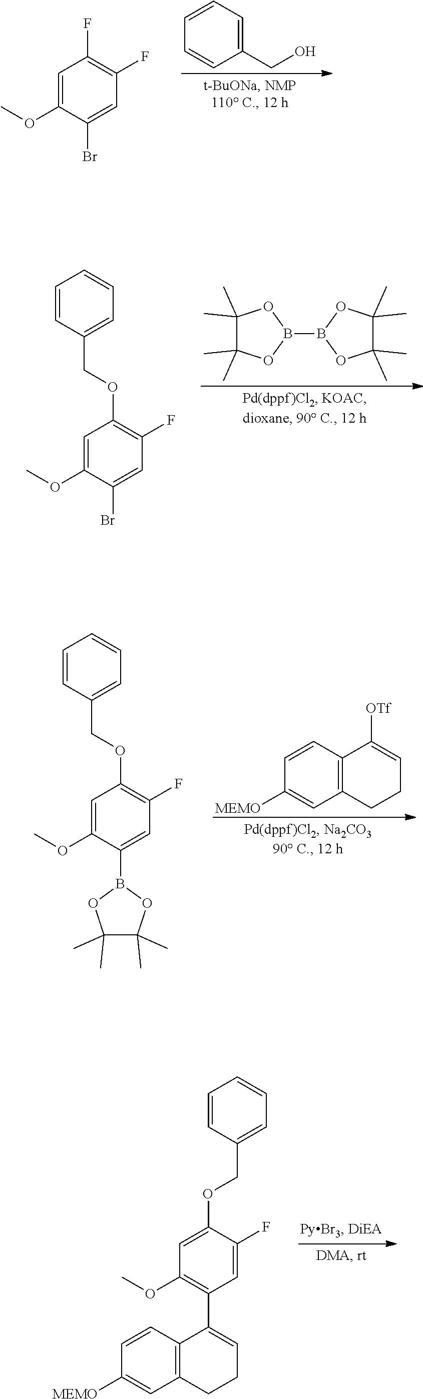

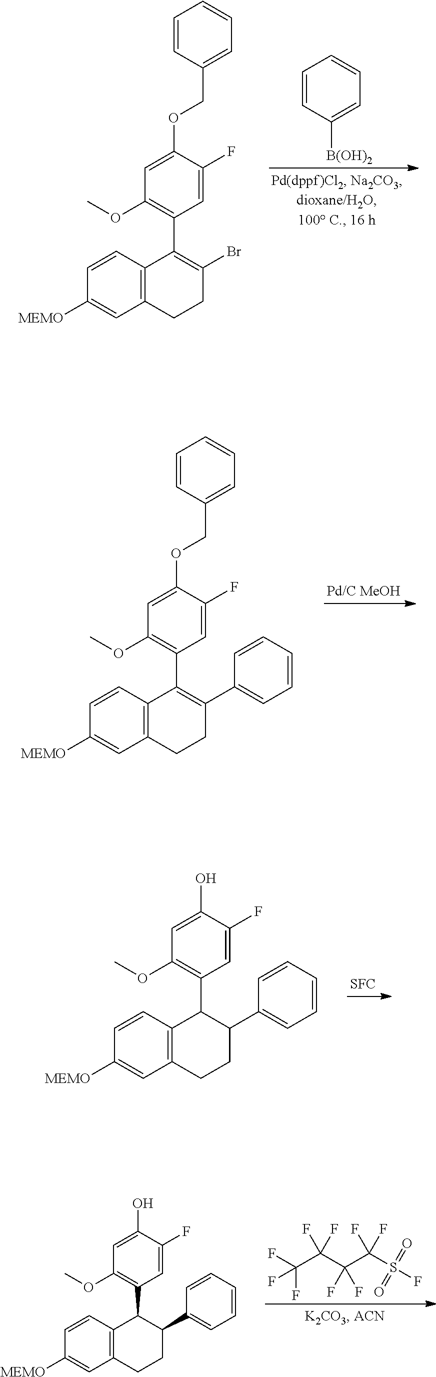

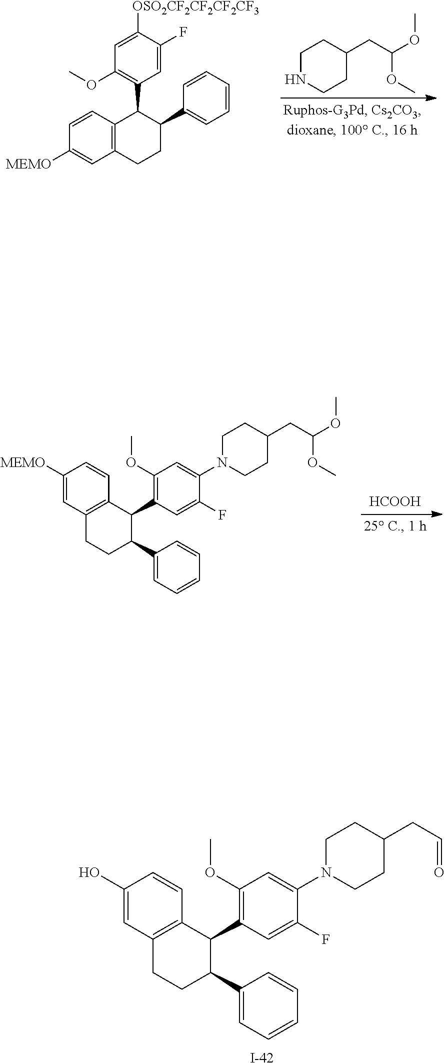

### Step 1:1-(benzyloxy)-4-bromo-2-fluoro-5-methoxybenzene

To a mixture of 1-bromo-4,5-difluoro-2-methoxybenzene (55.1 g, 1 eq, 247.1 mmol), phenylmethanol (29.4 g, 1,1 eq, 271.8 mmol) in NMP (200 mL) was added sodium tert-butoxide (28.5 g, 1,2 eq, 296.5 mmol). The reaction solution was stirred at 110° C. for 4 h under N₂. The reaction solution was quenched with NH₄Cl solution (1M, 300 mL), extracted with EA (400 mL×2). The combined organic layer was washed with brine (300 mL×3), dried over anhydrous Na₂SO₄, filtered and the filtrate was evaporated in vacuum. The residue was purified by column chromatography on silica gel eluted with (EA/PE=1:10) to give 1-(benzyloxy)-4-bromo-2-fluoro-5-methoxybenzene (65.0 g, 84.86%) as yellow oil. LC-MS (ESI, m/z): mass calcd. For C₁₅H₁₄BrFO₂, 310.0; found, 311,1 [M+H]⁺.

### Step 2:2-(4-(benzyloxy)-5-fluoro-2-methoxyphenyl)-4,4,5,5-tetramethyl-1,3,2-dioxaborolane

To a mixture of 1-(benzyloxy)-4-bromo-2-fluoro-5-methoxybenzene (65.52 g, 1 eq, 210.6 mmol), bis(pinacolato)diboron (80.21 g, 1.5 eq, 315.9 mmol), potassium acetate (41.33 g, 2 eq, 421,1 mmol) in 1,4-dioxane (300 mL) was added Pd(dppf)Cl₂ (3.1 g, 0.02 eq, 4.21 mmol) and the mixture was stirred at 100° C. for 12 h under N₂. The reaction solution was diluted with H₂O (300 mL), extracted with EA (200 mL×2). The combined organic layer was washed with brine (200 mL×3), dried over anhydrous Na₂SO₄, filtered and the filtrate was evaporated in vacuum.

The residue was purified by column chromatography on silica gel eluted with (PE:EA=10:1) to give 2-(4-(benzyloxy)-5-fluoro-2-methoxyphenyl)-4,4,5,5-tetramethyl-1,3,2-dioxaborolane (33.9 g, 44.9%) as a yellow solid. LC-MS (ESI, m/z): mass calcd. For C₂₀H₂₄BFO₄, 358.2; found, 359.4 [M+H]⁺.

### Step 3:4-(4-(benzyloxy)-5-fluoro-2-methoxyphenyl)-7-((2-methoxyethoxy)methoxy)-1,2-dihydronaphthalene

To a mixture of 2-(4-(benzyloxy)-5-fluoro-2-methoxyphenyl)-4,4,5,5-tetramethyl-1,3,2-dioxaborolane (3.50 g, 1 eq, 9.77 mmol), 6-((2-methoxyethoxy)methoxy)-3,4-dihydronaphthalen-1-yl trifluoromethanesulfonate (4,48 g, 1,2 eq, 11.7 mmol), Na₂CO₃ (2.07 g, 2 eq, 19.5 mmol) in 1,4-dioxane (35.0 mL)/H₂O (2.0 mL) was added Pd(dppf)Cl₂ (0.36 g, 0.05 eq, 488 μmol) and the mixture was stirred at 90° C. for 12 hours under N₂. The mixture was poured into H₂O (30 mL), extracted with EtOAc (20 mL×2). The combined organic layer was washed with brine (50 mL), dried over Na₂SO₄, filtered and concentrated in vacuum. The residue was purified by column chromatography on silica gel eluted with (PE:EA=5:1) to give 4-(4-(benzyloxy)-5-fluoro-2-methoxyphenyl)-7-((2-methoxyethoxy)methoxy)-1,2-dihydronaphthalene (1.70 g, 37.5%) as a yellow solid. LC-MS (ESI, m/z): mass calcd. For C₂₈H₂₉FO₅, 464.2; found, 465.3 [M+H]⁺. Step 4:4-(4-(benzyloxy)-5-fluoro-2-methoxyphenyl)-3-bromo-7-((2-methoxyethoxy)methoxy)-1,2-dihydronaphthalene

To a mixture of 4-(4-(benzyloxy)-5-fluoro-2-methoxyphenyl)-7-((2-methoxyethoxy)methoxy)-1,2-dihydronaphthalene (1.6 g, 3.5 mmol, 1 eq.) and DIEA (0.89 g, 6.9 mmol, 2 eq.) in DMA (10 mL), was added pyridinium tribromide (1.3 g, 4.2 mmol, 1,2 eq.) at 0° C. The mixture was stirred at room temperature for 3 h. The mixture was poured into H₂O (50 mL) and extracted with EtOAc (20 mL×2). The combined organic layer was washed with brine (40 mL), dried over Na₂SO₄, filtered and concentrated. The residue was purified by column chromatography on silica gel eluted with 0-20% EtOAc/hexane to afford 4-(4-(benzyloxy)-5-fluoro-2-methoxyphenyl)-3-bromo-7-((2-methoxyethoxy)methoxy)-1,2-dihydronaphthalene (1.5 g, 2.77 mmol, 791%) as a yellow solid. LC-MS (ESI, m/z): mass calcd. For C₂₈H₂₈BrFO₅, 542.1; found, 543.2 [M+H]⁺.

### Step 5:4-(4-(benzyloxy)-5-fluoro-2-methoxyphenyl)-7-((2-methoxyethoxy)methoxy)-3-phenyl-1,2-dihydronaphthalene

To a mixture of 4-(4-(benzyloxy)-5-fluoro-2-methoxyphenyl)-3-bromo-7-((2-methoxyethoxy)methoxy)-1,2-dihydronaphthalen (21.0 g, 1.0 eq, 38.6 mmol), phenylboronic acid (5,65 g, 1,2 eq, 46.4 mmol) in 1,4-Dioxane (100.0 mL)/H₂O (10.0 mL) was added Na₂CO₃ (8.19 g, 2.0 eq, 77.3 mmol) and PdCl₂(dppf) (1,41 g, 0.05 eq, 1.93 mmol). The reaction was stirred at 100° C. for 16 hours under N₂. The mixture was poured into H₂O (200 mL) and extracted with EtOAc (100 mL×2). The combined organic layer was washed with brine (150 mL), dried over Na₂SO₄, filtered and concentrated. The residue was purified by column chromatography on silica gel eluted with 0-30% EtOAc/hexane to afford 4-(4-(benzyloxy)-5-fluoro-2-methoxyphenyl)-7-((2-methoxyethoxy)methoxy)-3-phenyl-1,2-dihydronaphthalene (17.0 g, 31.4 mmol, 81.4%) as a yellow solid. LC-MS (ESI, m/z): mass calcd. For C₃₄H₃₃FO₅, 540.2: found, 541,4 [M+H]⁺.

### Step 6:2-fluoro-5-methoxy-4-(6-((2-methoxyethoxy)methoxy)-2-phenyl-1,2,3,4-tetrahydronaphthalen-1-yl)phenol

To a mixture of 4-(4-(benzyloxy)-5-fluoro-2-methoxyphenyl)-7-((2-methoxyethoxy)methoxy)-3-phenyl-1,2-dihydronaphthalene (17.0 g, 1 eq, 31.4 mmol) in MeOH (200 mL) was added Pd/C(5 g, 10% on Carbon, wetted with c.a,55% water). The mixture was stirred at room temperature overnight under H₂. The mixture was filtered and filtrate was concentrated. The residue was purified by column chromatography on silica gel eluted with 0-40% EtOAc/hexane to afford 2-fluoro-5-methoxy-4-(6-((2-methoxyethoxy)methoxy)-2-phenyl-1,2,3,4-tetrahydronaphthalen-1-yl)phenol (7.8 g, 17.3 mmol, 55%) as a yellow solid. LC-MS (ESI, m/z): mass calcd. For C₂₇H₂₉FO₅, 452.2; found, 453.3 [M+H]⁺.

2-fluoro-5-methoxy-4-(6-((2-methoxyethoxy)methoxy)-2-phenyl-1,2,3,4-tetrahydronaphthalen-1-yl)phenol (7.8 g) was purified by SFC to afford 2-fluoro-5-methoxy-4-((1R,2R)-6-((2-methoxyethoxy)methoxy)-2-phenyl-1,2,3,4-tetrahydronaphthalen-1-yl)phenyl 1,1,2,2,3,3,4,4,4-nonafluorobutane-1-sulfonate (3.5 g) as yellow solid and 2-fluoro-5-methoxy-4-((1S,2S)-6-((2-methoxyethoxy)methoxy)-2-phenyl-1,2,3,4-tetrahydronaphthalen-1-yl)phenyl 1,1,2,2,3,3,4,4,4-nonafluorobutane-1-sulfonate (3.5 g) as yellow solid. LC-MS (ESI, m/z): mass calcd. For C₂₇H₂₉FO₅, 452.2; found, 453.3 [M+H]⁺.

### Step 8:2-fluoro-5-methoxy-4-((1S,2S)-6-((2-methoxyethoxy)methoxy)-2-phenyl-1,2,3,4-tetrahydronaphthalen-1-yl)phenyl 1,1,2,2,3,3,4,4,4-nonafluorobutane-1-sulfonate

To a mixture of 2-fluoro-5-methoxy-4-((1S,2S)-6-((2-methoxyethoxy)methoxy)-2-phenyl-1,2,3,4-tetrahydronaphthalen-1-yl)phenol (0.50 g, 1 eq, 1,1 mmol) in MeCN (10.0 mL) was added K₂CO₃ (0.31 g, 2 eq, 2.2 mmol), 1,1,2,2,3,3,4,4,4-nonafluorobutane-1-sulfonyl fluoridc (0.43 g, 1.3 eq, 1.4 mmol) at 0° C. The mixture was stirred at room temperature for 16 hours. The mixture was concentrated under reduced pressure. The residue was purified by column chromatography on silica gel eluted with (PE:EA=5:1) to afford 2-fluoro-5-methoxy-4-((1S,2S)-6-((2-methoxyethoxy)methoxy)-2-phenyl-1,2,3,4-tetrahydronaphthalen-1-yl)phenyl 1,1,2,2,3,3,4,4,4-nonafluorobutane-1-sulfonate (0.81 g, 100%) as yellow oil. LC-MS (ESI, m/z): mass calcd. For C₃₁H₂₈F10O₇S, 734.1; found, 735.2 [M+H]⁺.

### Step 9:4-(2,2-dimethoxyethyl)-1-(2-fluoro-5-methoxy-4-((1S,2S)-6-((2-methoxyethoxy)methoxy)-2-phenyl-1,2,3,4-tetrahydronaphthalen-1-yl)phenyl)piperidine

To a mixture of 2-fluoro-5-methoxy-4-((1S,2S)-6-((2-methoxyethoxy)methoxy)-2-phenyl-1,2,3,4-tetrahydronaphthalen-1-yl)phenyl 1,1,2,2,3,3,4,4,4-nonafluorobutane-1-sulfonate (0.50 g, 1 eq, 0.68 mmol), 4-(2,2-dimethoxyethyl)piperidine (0.14 g, 1,2 eq, 0.82 mmol), Cs₂CO₃ (0.44 g, 2 eq, 0.68 mmol) in 1,4-dioxane (10.0 mL) was added RuPhos-Pd-G3 (57 mg, 0.1 eq, 0.068 mmol). The mixture was stirred at 100° C. for 16 h. The mixture was poured into H₂O (50 mL), extracted with EtOAc (20 mL×2). The combined organic layer was washed with brine (50 mL), dried over Na₂SO₄, filtered and concentrated in vacuum. The crude product was purified by column chromatography on silica gel using 0-30% EtOAc/hexane to afford 4-(2,2-dimethoxyethyl)-1-(2-fluoro-5-methoxy-4-((1S,2S)-6-((2-methoxyethoxy)methoxy)-2-phenyl-1,2,3,4-tetrahydronaphthalen-1-yl)phenyl)piperidine (0.15 g, 0.25 mmol, 36%) as a yellow solid. LC-MS (ESI, m/z): mass calcd. For C₃₋₆H₄₆FNO₆, 607.3; found, 608.4 [M+H]⁺.

### Step 10:2-(1-(2-fluoro-4-((1,2S)-6-hydroxy-2-phenyl-1,2,3,4-tetrahydronaphthalen-1-yl)-5-methoxyphenyl)piperidin-4-yl)acetaldehyde

To a mixture of 4-(2,2-dimethoxyethyl)-1-(2-fluoro-5-methoxy-4-((1S,2S)-6-((2-methoxyethoxy)methoxy)-2-phenyl-1,2,3,4-tetrahydronaphthalen-1-yl)phenyl)piperidine (0.03 g, 1 eq, 0.05 mmol) in HCOOH (2.00 mL). The mixture was stirred at room temperature for 1 h, the mixture was concentrated to afford 2-(1-(2-fluoro-4-((1S,2S)-6-hydroxy-2-phenyl-1,2,3,4-tetrahydronaphthalen-1-yl)-5-methoxyphenyl)piperidin-4-yl)acetaldehyde (30.0 mg, crude) as yellow oil. LC-MS (ESI, m/z): mass calcd. For C₃₀H₃₂FNO₃, 473.2; found, 474.3 [M+H]⁺.

### Intermediate 43:1-(4-((1R,2S)-6-(tert-butoxy)-2-phenyl-1,2,3,4-tetrahydronaphthalen-1-yl)phenyl)-4-(2,2-dimethoxyethyl)-4-methoxypiperidine

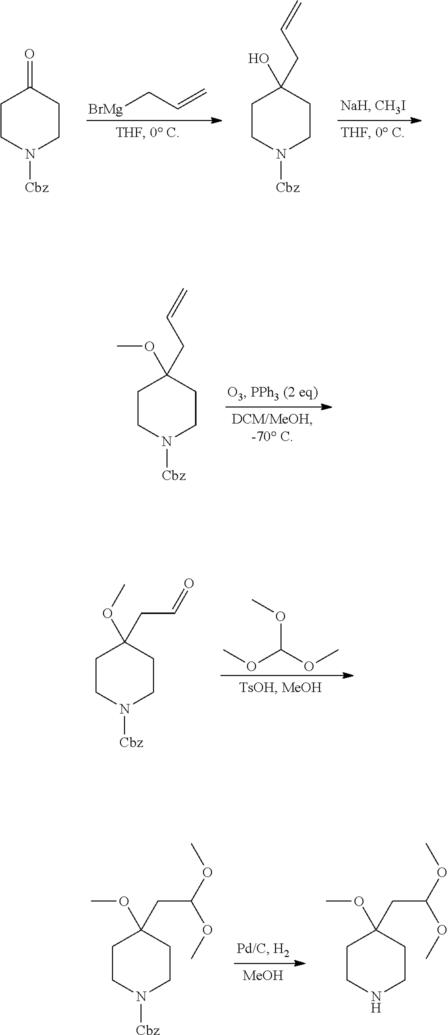

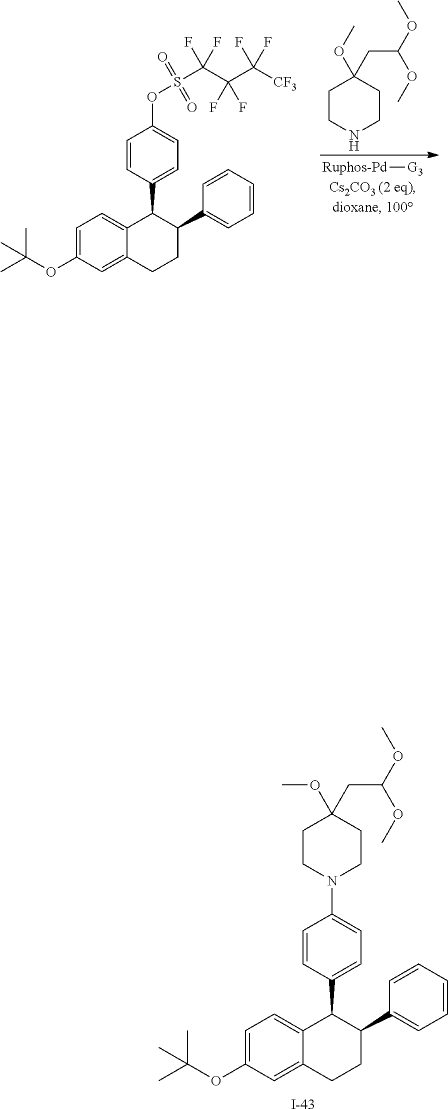

### Step 1: benzyl 4-allyl-4-hydroxypiperidine-1-carboxylate

To a mixture of allylmagnesium bromide (171,4 mL, 1 M in THF, 2 eq, 171,4 mmol) in THF (120.0 mL) at 0° C. was added benzyl 4-oxopiperidine-1-carboxylate (20.00 g, 1 eq, 85.8 mmol) in THF (40.0 mL) dropwise slowly at 0° C. over 30 minutes. The mixture was stirred at 0° C. for 5 hours, then quenched with Sat. NH₄Cl solution (40 mL) and water (200 mL), extracted with EtOAc (120 mL×3). The combined organic layers were washed with water (100 mL×2) and brine (200 mL) successively, then dried over anhydrous Na₂SO₄, filtered and concentrated under reduced pressure. The residue was purified by reversed Chem-flash to afford benzyl 4-allyl-4-hydroxypiperidine-1-carboxylate (9.2 g, 39.0%) as yellow oil. LC-MS (ESI, m/z): mass calcd. For C₁₋₆H₂₁NO₃, 275.15; found, 276.3 [M+H]⁺.

### Step 2: benzyl 4-allyl-4-methoxypiperidine-1-carboxylate

To a mixture of benzyl 4-allyl-4-hydroxypiperidine-1-carboxylate (7.00 g, 1 eq, 25.4 mmol) in THF (40.0 mL) was added sodium hydride (3.05 g, 60% wt, 3 eq, 76.3 mmol) slowly at 0° C. under N₂. The mixture was stirred for 1.5 hours, then added with iodomethane (5.41 g, 1.5 eq, 38.1 mmol) dropwise slowly at 0° C. The resulting mixture was stirred at rt for 6 hours, then quenched with Sat. NH₄Cl solution (50 mL) and water (50 mL), extracted with EtOAc (100 mL×3). The combined organic layers were washed with water (60 mL×2) and brine (100 mL) successively, dried over anhydrous Na₂SO₄, filtered and concentrated under reduced pressure. The crude product was purified by column chromatography on silica gel eluted with (PE:EtOAc=5:1) to afford benzyl 4-allyl-4-methoxypiperidine-1-carboxylate (6.80 g, 23.5 mmol, 92.4%) as a white oil. LC-MS (ESI, m/z): mass calcd. For C₁₇H₂₃NO₃, 289.17; found, 290.2 [M+H]⁺.

### Step 3: benzyl 4-methoxy-4-(2-oxoethyl)piperidine-1-carboxylate

An ozone-enriched steam of oxygen was bubbled through the solution of benzyl 4-allyl-4-methoxypiperidine-1-carboxylate (6.80 g, 1 eq, 23.5 mmol) in DCM (32.0 mL) and MeOH (6.00 mL) at −40° C., until the solution became light blue. Then the solution was purged with argon at-40° C. for 10 minutes to remove excess O₃, then added with triphenylphosphane (6.16 g, 1 eq, 23.5 mmol) slowly at −20° C. The resulting mixture was stirred at 25° C. for 1 hour, then poured into ice water (100 mL), extracted with EtOAc (100 mL×3). The organic layer was washed with brine, dried over anhydrous Na₂SO₄, filtered and concentrated. The crude product was purified by column chromatography on silica gel eluted with (PE:EtOAc=5:1) to afford benzyl 4-methoxy-4-(2-oxoethyl)piperidine-1-carboxylate (5.00 g, 17.2 mmol, 73.0%) as a yellow oil. LC-MS (ESI, m/z): mass calcd. For C₁₋₆H₂₁NO₄, 291,15; found, 292.2 [M+H]⁺.

### Step 4: benzyl 4-(2,2-dimethoxyethyl)-4-methoxypiperidine-1-carboxylate

A mixture of benzyl 4-methoxy-4-(2-oxoethyl)piperidine-1-carboxylate (5.00 g, 1 eq, 17.2 mmol), trimethoxymethane (3.64 g, 2 eq, 34.3 mmol) and 4-methylbenzenesulfonic acid hydrate (163 mg, 0.05 eq, 858 μmol) in MeOH (50.0 mL) was stirred at 60° C. for 12 hours. LC-MS showed the reaction was completed. The mixture was poured into ice water (100 mL), extracted with EtOAc (100 mL×2). The organic layer was washed with brine (100 mL), then dried over anhydrous Na₂SO₄, filtered and concentrated. The crude product was purified by column chromatography on silica gel eluted with (PE:EtOAc=5:1) to afford benzyl 4-(2,2-dimethoxyethyl)-4-methoxypiperidine-1-carboxylate (4.50 g, 13.3 mmol, 77.7%) as a white viscous solid. LC-MS (ESI, m/z): mass calcd. For C18H₂₇NO₅, 337.19; found, 338.2 [M+H]⁺.

### Step 5:4-(2,2-dimethoxyethyl)-4-methoxypiperidine

To a mixture of benzyl 4-(2,2-dimethoxyethyl)-4-methoxypiperidine-1-carboxylate (4.50 g, 1 Eq, 13.3 mmol) in MeOH (50.0 mL) was added Pd/C(946 mg, 10% on Carbon, wetted with ca, 55% water)

The suspension was degassed and charged with H₂ three times. The mixture was stirred at 25° C. for 14 hours. TLC showed was completed. The mixture was filtered and the filtered concentrated under reduced pressure to afford the 4-(2,2-dimethoxyethyl)-4-methoxypiperidine (2.50 g, 12,3 mmol, 92.2%) as gray oil. LC-MS (ESI, m/z): mass calcd. For C₁₀H₂₁NO₃, 203.15; found.204.2 [M+H]⁺.

### Step 6:1-(4-((1R,2S)-6-(tert-butoxy)-2-phenyl-1,2,3,4-tetrahydronaphthalen-1-yl)phenyl)-4-(2,2-dimethoxyethyl)-4-methoxypiperidine

To a mixture of 4-((1R,2S)-6-(tert-butoxy)-2-phenyl-1,2,3,4-tetrahydronaphthalen-1-yl)phenyl 1,1,2,2,3,3,4,4,4-nonafluorobutanc-1-sulfonate (0.30 g, 1 eq, 0.46 mmol), 4-(2,2-dimethoxyethyl)-4-methoxypiperidine (0.14 g, 1.5 eq, 0.69 mmol) and K₂CO₃ (63 mg, 1 eq, 0.46 mmol) in 1,4-Dioxane (4.00 mL) was added Ruphos-Pd-G3 (38 mg, 0.1 eq, 46 μmol). The resulting mixture was stirred at 100° C. under N₂ for 12 hours. LCMS showed compound the desired product was formed. The mixture was poured into ice water (10 mL), extracted with EA (20 mL×2). The organic layer was washed with brine (20 mL), dried over anhydrous Na₂SO₄, filtered and concentrated. The crude product was purified by column chromatography on silica gel eluted with (PE:EtOAc=5:1) to afford 1-(4-((1R,2S)-6-(tert-butoxy)-2-phenyl-1,2,3,4-tetrahydronaphthalen-1-yl)phenyl)-4-(2,2-dimethoxyethyl)-4-methoxypiperidine (0.14 g, 55%) as a white solid. LC-MS (ESI, m/z): mass calcd. For C₃₋₆H₄₇NO₄, 557.35; found, 558.4 [M+H]⁺.

### Intermediate 44:1-(4-((1R,2S)-6-(tert-butoxy)-2-phenyl-1,2,3,4-tetrahydronaphthalen-1-yl)phenyl)-4-(dimethoxymethyl)-4-methoxypiperidine

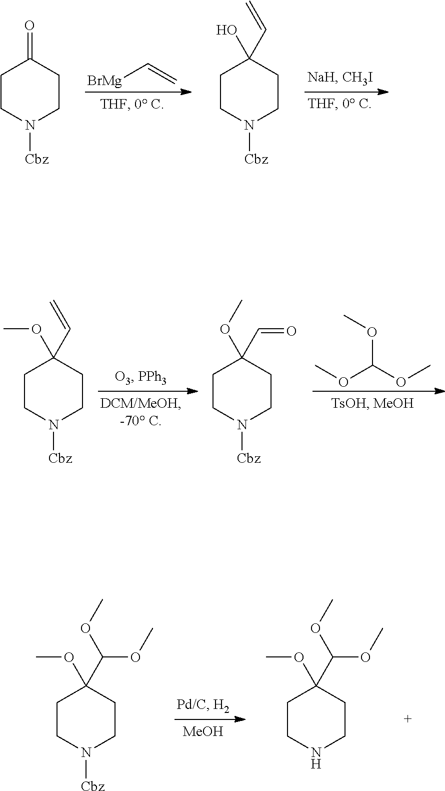

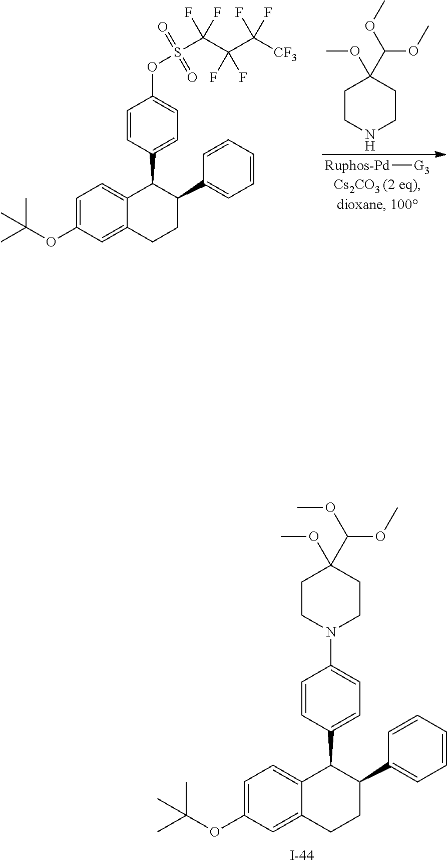

The synthesis of 4-(dimethoxymethyl)-4-methoxypiperidine is similar to that of 4-(2,2-dimethoxyethyl)-4-methoxypiperidine.

### Step 1:1-(4-((1R,2S)-6-(tert-butoxy)-2-phenyl-1,2,3,4-tetrahydronaphthalen-1-yl)phenyl)-4-(dimethoxymethyl)-4-methoxypiperidine

A mixture of 4-((1R,2S)-6-(tert-butoxy)-2-phenyl-1,2,3,4-tetrahydronaphthalen-1-yl)phenyl 1,1,2,2,3,3,4,4,4-nonafluorobutane-1-sulfonate (250 mg, 1 eq, 382 μmol), 4-(dimethoxymethyl)-4-methoxypiperidine (108 mg, 1.5 Eq, 573 μmol), Cs₂CO₃ (249 mg, 2 eq, 764 μmol) and Ruphos-Pd-G3 (320 mg, 1 eq, 382 μmol) in 1,4-Dioxane (6.00 mL) was stirred at 100° C. under N₂ for 12 hour. LCMS showed compound the desired product was formed. The mixture was poured into ice water (10 mL), extracted with EA (20 mL×2). The organic layer was washed with brine (20 mL), dried over anhydrous Na₂SO₄, filtered and concentrated. The crude product was purified by column chromatography on silica gel eluted with (PE:EtOAc=5:1) to afford 1-(4-((1R,2S)-6-(tert-butoxy)-2-phenyl-1,2,3,4-tetrahydronaphthalen-1-yl)phenyl)-4-(dimethoxymethyl)-4-methoxypiperidine (120 mg, 57.8%) as a white solid. LC-MS (ESI, m/z): mass calcd. For C₃₅H₄₅NO₄, 543.33; found, 544,4 [M+H]⁺.

### Intermediate 45:2-(1-(2-fluoro-4-((1S,2S)-6-hydroxy-2-phenyl-1,2,3,4-tetrahydronaphthalen-1-yl)-5-methoxyphenyl) azetidin-3-yl)acetaldehyde

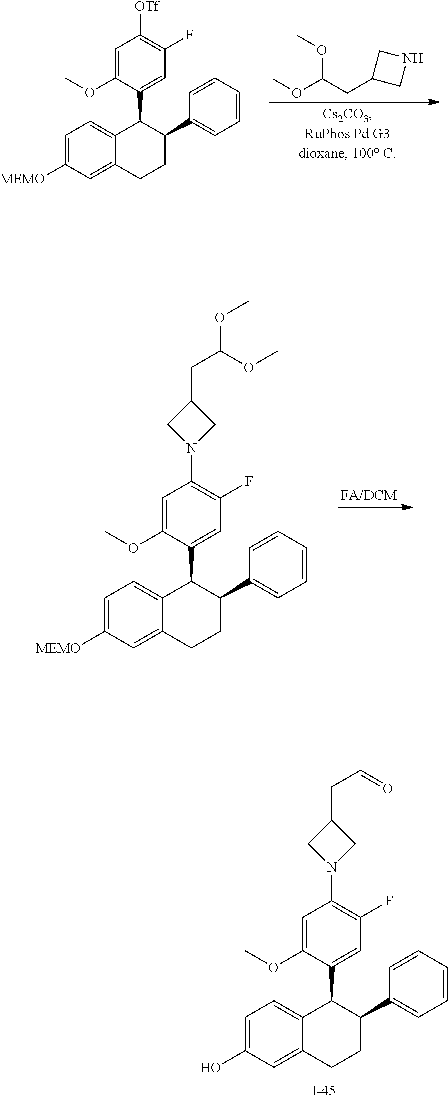

### Step 1:3-(2,2-dimethoxyethyl)-1-(2-fluoro-5-methoxy-4-((1S,2S)-6-((2-methoxyethoxy)methoxy)-2-phenyl-1,2,3,4-tetrahydronaphthalen-1-yl)phenyl) azetidine

To a solution of 2-fluoro-5-methoxy-4-((1S,2S)-6-((2-methoxyethoxy)methoxy)-2-phenyl-1,2,3,4-tetrahydronaphthalen-1-yl)phenyl trifluoromethanesulfonate (639 mg, 1.09 mmol, 1 eq) and 3-(2,2-dimethoxyethyl) azetidine (175 mg, 1,20 mmol, 1,1 eq) in 1,4-Dioxane (10.0 mL) was added Cs₂CO₃ (1.78 g, 5.47 mmol, 5 eq) and RuPhos Pd G3 (7.15 mg, 1.09 mmol, 1 eq). The solution was stirred at 100° C. for 16 hours. The reaction mixture was quenched with addition H₂O (30 mL), and extracted with DCM (10 mL×3). The combined organic layers were washed with brine (10 mL×3), dried over Na₂SO₄, filtered and concentrated under reduced pressure. The residue was purified by flash silica gel chromatography (0˜30% Ethyl acetate/Petroleum ether) to afford 3-(2,2-dimethoxyethyl)-1-(2-fluoro-5-methoxy-4-((1S,2S)-6-((2-methoxyethoxy)methoxy)-2-phenyl-1,2,3,4-tetrahydronaphthalen-1-yl)phenyl) azetidine (568 mg, 89.6%) was obtained as yellow liquid. LC-MS (ESI, m/z): mass calcd. For C₃₄H₄₂FNO₆, 579.3; found, 580.1 [M+H]⁺.

### Step 2:2-(1-(2-fluoro-4-((1S,2S)-6-hydroxy-2-phenyl-1,2,3,4-tetrahydronaphthalen-1-yl)-5-methoxyphenyl) azetidin-3-yl)acetaldehyde

To a solution of 3-(2,2-dimethoxyethyl)-1-(2-fluoro-5-methoxy-4-((1S,2S)-6-((2-methoxyethoxy)methoxy)-2-phenyl-1,2,3,4-tetrahydronaphthalen-1-yl)phenyl) azetidine (568 mg, 980 μmol, 1 eq) in DCM (5.00 mL) was added formic acid (90.2 mg, 1.96 mmol, 2 eq). The solution was stirred at 25° C. for 2 hours. The reaction mixture was quenched with H₂O (5.0 mL×3), and extracted with DCM (5.0 mL×3). The combined organic layers were washed with brine (5.0 mL×3), dried over Na₂SO₄, filtered and concentrated under reduced pressure. The residue was purified by flash silica gel chromatography (0˜30% Ethyl acetate/Petroleum ether) to afford 2-(1-(2-fluoro-4-((1S,2S)-6-hydroxy-2-phenyl-1,2,3,4-tetrahydronaphthalen-1-yl)-5-methoxyphenyl) azetidin-3-yl)acetaldehyde (460 mg, 88.0%) was obtained as a yellow solid. LC-MS (ESI, m/z): mass calcd. For C₃₂H₃₆FNO₅, 533.26; found, 534.3 [M+H]⁺.

### Intermediate 46: (S)-3-(5-methyl-6-oxo-6,8-dihydro-2H,7H-spiro[furo[2,3-e]isoindole-3,4′-piperidin]-7-yl)piperidine-2,6-dione

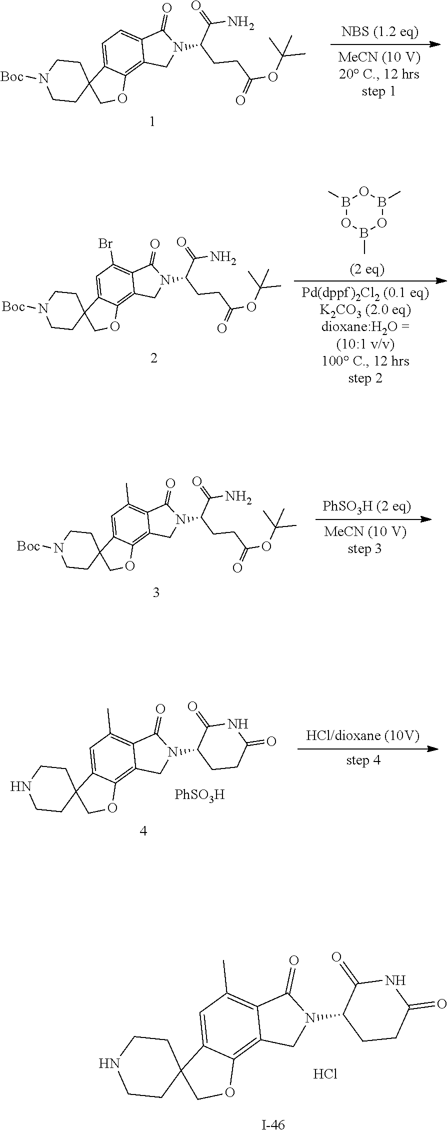

### Step 1: tert-butyl(S)-7-(1-amino-5-(tert-butoxy)-1,5-dioxopentan-2-yl)-5-bromo-6-oxo-7,8-dihydro-2H,6H-spiro[furo[2,3-e]isoindole-3,4′-piperidine]-1′-carboxylate

To a solution of tert-butyl(S)-7-(1-amino-5-(tert-butoxy)-1,5-dioxopentan-2-yl)-6-oxo-7,8-dihydro-2H,6H-spiro[furo[2,3-e]isoindole-3,4′-piperidine]-1′-carboxylate (20 g, 37.8 mmol, 1 eq) in MeCN (200 mL) at 20° C. was added NBS (8.07 g, 45.3 mmol, 1,2 eq) in portions at 10° C. and the resulting mixture was kept stirring at 20° C. for 12 hrs. TLC analysis (Petroleum ether: Ethyl acetate=0:1, R_{f}=0.51) showed tert-butyl(S)-7-(1-amino-5-(tert-butoxy)-1,5-dioxopentan-2-yl)-6-oxo-7,8-dihydro-2H,6H-spiro[furo[2,3-e]isoindole-3,4′-piperidine]-1′-carboxylate was completely consumed and the mixture was concentrated in vacuum. The residue was purified by silica gel chromatography (column height: 250 mm, diameter: 100 mm, 100-200 mesh silica gel, Petroleum ether/Ethyl acetate=100/1, 1/1) to yield tert-butyl(S)-7-(1-amino-5-(tert-butoxy)-1,5-dioxopentan-2-yl)-5-bromo-6-oxo-7,8-dihydro-2H,6H-spiro[furo[2,3-e]isoindole-3,4′-piperidine]-1′-carboxylate (18 g, 25.1 mmol, 66.6% yield) was obtained as a yellow solid.

¹H NMR: (400 MHZ, CDCl₃) δ 8.68-8.54 (m, 1H), 7.30 (s, 1H), 6.51 (br s, 1H), 5,68 (br s, 1H), 4.90 (dd, J=6.2, 8.8 Hz, 1H), 4.54 (s, 2H), 4.51-4,29 (m, 2H), 4.12 (q. J=7.1 Hz, 2H), 2.87 (br t, J=12.2 Hz, 2H), 2.35-2.23 (m, 2H), 2.21-2.09 (m, 1H), 1.92-1.81 (m, 2H), 1.80-1.68 (m, 3H), 1.49 (s, 9H), 1.42 (s, 9H). LC/MS (MH⁺): 608.2.

### Step 2: tert-butyl(S)-7-(1-amino-5-(tert-butoxy)-1,5-dioxopentan-2-yl)-5-methyl-6-oxo-7,8-dihydro-2H,6H-spiro[furo[2,3-e]isoindole-3,4′-piperidine]-1′-carboxylate

(S)-7-(1-amino-5-(tert-butoxy)-1,5-dioxopentan-2-yl)-5-bromo-6-oxo-7,8-dihydro-2H,6H-spiro[furo[2,3-e]isoindole-3,4′-piperidine]-1′-carboxylate (18 g, 29.6 mmol, 1 eq) was dissolved in dioxane (150 mL) and H₂O (15 mL) at 20° C. To which was added 2,4,6-trimethyl-1,3,5,2,4,6-trioxatriborinane (7.43 g, 59.2 mmol, 8.27 mL, 2 eq), K₂CO₃ (12,3 g, 88.7 mmol, 3 eq) and Pd(dppf)Cl₂ (4.33 g, 5.92 mmol, 0.2 eq) in portions at 20° C. under N₂. The reaction mixture was stirred at 80° C. for 12 hrs under N₂. LCMS showed that(S)-7-(1-amino-5-(tert-butoxy)-1,5-dioxopentan-2-yl)-5-bromo-6-oxo-7,8-dihydro-2H,6H-spiro[furo[2,3-e]isoindole-3,4′-piperidine]-1′-carboxylate was consumed completely and the mixture was diluted with water (100 mL) and extracted with EtOAc (50 mL×2). The combined organic layers were washed with brine (100 mL), dried over anhydrous Na₂SO₄, and concentrated under reduced pressure. The residue was purified by silica gel chromatography (column height: 250 mm, diameter: 100 mm, 100-200 mesh silica gel, Petroleum ether/Ethyl acetate=100/1, 1/1) to yield tert-butyl(S)-7-(1-amino-5-(tert-butoxy)-1,5-dioxopentan-2-yl)-5-methyl-6-oxo-7,8-dihydro-2H,6H-spiro[furo[2,3-e]isoindole-3,4′-piperidine]-1′-carboxylate as a yellow solid (9.5 g, 15.7 mmol, 53.2% yield).

¹H NMR (400 MHZ, CDCl₃) δ 6.95 (s, 1H), 6.30 (br s, 1H), 5.40 (br s, 1H), 4.94-4.79 (m, 1H), 4.49 (s, 2H), 4.43-4,28 (m, 2H), 4.16-4.08 (m, 2H), 2.88 (br t, J=12,3 Hz, 2H), 2.64 (s, 3H), 2.43-2.20 (m, 3H), 2.18-2.08 (m, 1H), 1.94-1.81 (m, 2H), 1.72 (br d. J=13,4 Hz, 2H), 1.50 (s, 8H), 1.42 (s, 8H). LC/MS (MH⁺): 544,4.

### Step 3: (S)-3-(5-methyl-6-oxo-6,8-dihydro-2H,7H-spiro[furo[2,3-e]isoindole-3,4′-piperidin]-7-yl)piperidine-2,6-dione benzenesulfonate

PhSO₃H (5.24 g, 33.1 mmol, 2 eq) was dissolved in MeCN (100 mL) at 20° C. To which was add a solution of tert-butyl(S)-7-(1-amino-5-(tert-butoxy)-1,5-dioxopentan-2-yl)-5-methyl-6-oxo-7,8-dihydro-2H,6H-spiro[furo[2,3-e]isoindole-3,4′-piperidine]-1′-carboxylate (9 g, 16.6 mmol, 1 eq) in MeCN (50 mL) drop-wise. The reaction mixture was stirred at 100° C. for 12 hrs. Upon the reaction is completed, the mixture was filtered, and the cake was concentrated under reduced pressure. (S)-3-(5-methyl-6-oxo-6,8-dihydro-2H,7H-spiro[furo[2,3-e]isoindole-3,4′-piperidin]-7-yl)piperidine-2,6-dione benzenesulfonate (6.6 g, 10.0 mmol, 60.5% yield, PhSO₃H) was obtained as a white solid.

¹H NMR: (400 MHZ, D₂O) δ 7.75 (br d, J=6.8 Hz, 2H), 7.58-7.40 (m, 3H), 7.19 (s, 1H), 5.14-4.96 (m, 1H), 4.59 (s, 2H), 4.48-4,27 (m, 2H), 3.48 (br d, J=13.0 Hz, 2H), 3.12 (br t, J=12,3 Hz, 2H), 2.98-2,77 (m, 2H), 2.53 (s, 3H), 2.48 (br dd, J=5.4, 13.1 Hz, 1H), 2.25-2.05 (m, 3H), 2.01-1.90 (m, 2H). LC/MS (MH⁺): 370.2.

### Step 4: (S)-3-(5-methyl-6-oxo-6,8-dihydro-2H,7H-spiro[furo[2,3-e]isoindole-3,4′-piperidin]-7-yl)piperidine-2,6-dione HCl salt

(S)-3-(5-methyl-6-oxo-6,8-dihydro-2H,7H-spiro[furo[2,3-e]isoindole-3,4′-piperidin]-7-yl)piperidine-2,6-dione benzenesulfonate (6.6 g, 12.5 mmol, 1 eq, PhSO₃H) was dissolved in HCl/dioxane (60 mL) at 20° C. and the mixture was stirred at 20° C. for 12 hrs. The mixture was filtered, and the cake was washed by MeCN (50 mL×2) and concentrated under reduced pressure. (S)-3-(5-methyl-6-oxo-6,8-dihydro-2H,7H-spiro[furo[2,3-e]isoindole-3,4′-piperidin]-7-yl)piperidine-2,6-dione HCl salt (4.8 g, 10.6 mmol, 85.1% yield) was obtained as a white solid.

¹H NMR (400 MHZ, D₂O) δ 7.26-7.10 (m, 1H), 5.15-5.03 (m, 1H), 4.61 (s, 2H), 4.41 (br d, J=16.5 Hz, 2H), 3.50 (br d, J=12.8 Hz, 2H), 3.13 (br t, J=12.5 Hz, 2H), 2.95-2,78 (m, 2H), 2.54 (s, 3H), 2.51-2.42 (m, 1H), 2.26-2.09 (m, 3H), 2.04-1.94 (m, 2H),

### Intermediate 47: (R)-9-(4-((1S,2S)-6-(tert-butoxy)-2-phenyl-1,2,3,4-tetrahydronaphthalen-1-yl)-2-fluoro-5-methoxyphenyl)-3-(dimethoxymethyl)-1-oxa-9-azaspiro[5.5]undecane

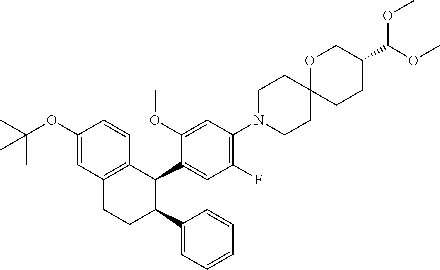 [SMILES: COc1cc(N2CCC3(CC[C@@H](C(OC)OC)CO3)CC2)c(F)cc1[C@@H]1c2ccc(OC(C)(C)C)cc2CC[C@@H]1c1ccccc1]

To a solution of 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutane-1-sulfonate (1.50 g, 1 Eq, 2.13 mmol), (R)-3-(dimethoxymethyl)-1-oxa-9-azaspiro[5.5]undecane (636 mg, 1.3 Eq, 2.78 mmol), Xphos (102 mg, 0.1 Eq, 213 μmol) and Cs₂CO₃ (1.39 g, 2 Eq, 4.27 mmol) in dioxane (15 mL) was added Pd₂(dba) 3 (196 mg, 0.1 Eq, 213 μmol). The mixture was stirred at 100° C. for 24 hours. TLC (Petroleum ether: Ethyl acetate=5:1, R_{f}=0.68) indicated 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutane-1-sulfonate was consumed completely, and one new spot formed. The reaction mixture was filtered and concentrated under reduced pressure to give a residue. The residue was purified by silica gel chromatography (column height: 250 mm, diameter: 100 mm, 100-200 mesh silica gel, Petroleum ether/Ethyl acetate=100/1, 1/1). (R)-9-(4-((1S,2S)-6-(tert-butoxy)-2-phenyl-1,2,3,4-tetrahydronaphthalen-1-yl)-2-fluoro-5-methoxyphenyl)-3-(dimethoxymethyl)-1-oxa-9-azaspiro[5.5]undecane (0.80 g, 1,2 mmol, 57% yield) was obtained as a yellow solid, ¹H NMR (400 MHZ, CHLOROFORM-d) δ =7.13-7.03 (m, 3H), 6.86-6.75 (m, 4H), 6.74-6.68 (m, 1H), 6.31 (d, J=13.7 Hz, 1H), 6.13 (d, J=7.5 Hz, 1H), 4.83 (br d, J=5.4 Hz, 1H), 4.17 (d, J=7.5 Hz, 1H), 3.74 (br dd, J=4.0, 12.1 Hz, 1H), 3.49-3.40 (m, 1H), 3.34 (d, J=1.5 Hz, 7H), 3.11-2.93 (m, 8H), 2.87-2,77 (m, 1H), 2.33-2.13 (m, 2H), 1.98-1.87 (m, 1H), 1.81-1.69 (m, 4H), 1.63-1.57 (m, 1H), 1.53-1.42 (m, 3H), 1.36 (s, 9H), m/z+1=631.8

### Intermediate 48: (S)-9-(4-((1S,2S)-6-(tert-butoxy)-2-phenyl-1,2,3,4-tetrahydronaphthalen-1-yl)-2-fluoro-5-methoxyphenyl)-3-(dimethoxymethyl)-1-oxa-9-azaspiro[5.5]undecane

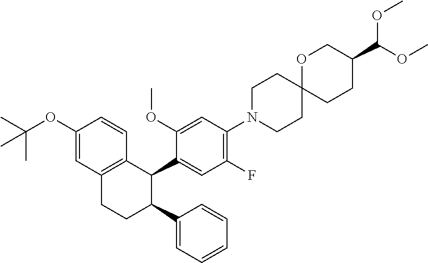 [SMILES: COc1cc(N2CCC3(CC[C@H](C(OC)OC)CO3)CC2)c(F)cc1[C@@H]1c2ccc(OC(C)(C)C)cc2CC[C@@H]1c1ccccc1]

To a solution of 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutanc-1-sulfonatc (1.50 g, 1 Eq, 2.13 mmol), (S)-3-(dimethoxymethyl)-1-oxa-9-azaspiro[5.5]undecane (636 mg, 1.3 Eq, 2.78 mmol), Xphos (102 mg, 0.1 Eq, 213 μmol) and Cs₂CO₃ (1.39 g, 2 Eq, 4.27 mmol) in dioxane (15 mL) was added Pd₂(dba) 3 (196 mg, 0.1 Eq, 213 μmol). The mixture was stirred at 100° C. for 24 hours. TLC (Petroleum ether: Ethyl acetate=5:1, R_{f}=0.68) indicated 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutane-1-sulfonate was consumed completely, and one new spot formed. The reaction mixture was filtered and concentrated under reduced pressure to give a residue. The residue was purified by silica gel chromatography (column height: 250 mm, diameter: 100 mm, 100-200 mesh silica gel, Petroleum ether/Ethyl acetate=100/1, 1/1). (S)-9-(4-((1S,2S)-6-(tert-butoxy)-2-phenyl-1,2,3,4-tetrahydronaphthalen-1-yl)-2-fluoro-5-methoxyphenyl)-3-(dimethoxymethyl)-1-oxa-9-azaspiro[5.5]undecane (0.70 g, 1,1 mmol, 51% yield) was obtained as a yellow solid, ¹H NMR (400 MHZ, CHLOROFORM-d) δ =7.14-7.03 (m, 3H), 6.88-6.68 (m, 5H), 6.31 (d, J=13.7 Hz, 1H), 6.12 (d, J=7.5 Hz, 1H), 4.83 (br d, J=5.3 Hz, 1H), 4.19 (d, J=7.5 Hz, 1H), 3.75 (br dd, J=3.7, 12.1 Hz, 1H), 3.47 (dd, J=9.8, 11.7 Hz, 1H), 3.39-3,25 (m, 7H), 3.11-2.93 (m, 8H), 2.92-2.85 (m, 1H), 2.33-2.14 (m, 2H), 1.98-1.86 (m, 1H), 1.80-1.68 (m, 4H), 1.63-1.57 (m, 2H), 1.54-1.43 (m, 2H), 1.36 (s, 9H), m/z+1-631.8

### Intermediate 49: (R)-9-(4-((1R,2S)-6-(tert-butoxy)-2-phenyl-1,2,3,4-tetrahydronaphthalen-1-yl)phenyl)-3-(dimethoxymethyl)-1-oxa-9-azaspiro[5.5]undecane

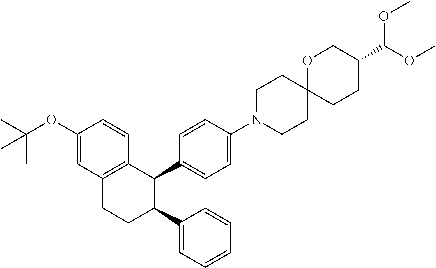 [SMILES: COC(OC)[C@@H]1CCC2(CCN(c3ccc([C@@H]4c5ccc(OC(C)(C)C)cc5CC[C@@H]4c4ccccc4)cc3)CC2)OC1]

To a solution of 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutane-1-sulfonate (3.00 g, 1 Eq, 4.58 mmol), (R)-3-(dimethoxymethyl)-1-oxa-9-azaspiro[5.5]undecane (1.37 g, 1.3 Eq, 5.96 mmol), Xphos (218 mg, 0.1 Eq, 458 μmol) and Cs₂CO₃ (2.99 g, 2 Eq, 9.17 mmol) in dioxane (30 mL) was added Pd₂(dba) 3 (420 mg, 0.1 Eq, 458 μmol). The mixture was stirred at 100° C. for 24 hours. TLC (Petroleum ether: Ethyl acetate=5:1, R_{f}=0.68) indicated 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutanc-1-sulfonate was consumed completely, and one new spot formed. The reaction mixture was filtered and concentrated under reduced pressure to give a residue. The residue was purified by silica gel chromatography (column height: 250 mm, diameter: 100 mm, 100-200 mesh silica gel, Petroleum ether/Ethyl acetate=100/1, 1/1). (R)-9-(4-((1R,2S)-6-(tert-butoxy)-2-phenyl-1,2,3,4-tetrahydronaphthalen-1-yl)phenyl)-3-(dimethoxymethyl)-1-oxa-9-azaspiro[5.5]undecane (1.60 g, 2.7 mmol, 59%) was obtained as a yellow solid, ¹H NMR (400 MHZ, CHLOROFORM-d) δ =7.21-7.09 (m, 3H), 6.89-6.77 (m, 4H), 6.73 (dd, J=2.2. 8.3 Hz, 1H), 6.58 (d, J=8.7 Hz, 2H), 6.27 (d, J=8.6 Hz, 2H), 4.23 (br d. J=4.8 Hz, 1H), 4.16 (d, J=7.3 Hz, 1H), 3.72 (br dd, J=4.0, 11.9 Hz, 1H), 3.44 (dd, J=10.1, 11.8 Hz, 1H), 3.38 (br d, J=4.8 Hz, 7H), 3.24-3.14 (m, 2H), 3.11-2.98 (m, 3H), 2.97-2.87 (m, 1H), 2.25-2.08 (m, 2H), 1.98-1.86 (m, 1H), 1.84-1.76 (m, 1H), 1.76-1.66 (m, 3H), 1.59 (br d, J=4.5 Hz, 1H), 1.53-1.41 (m, 3H), 1.37 (s, 9H), m/z+1=583.8

### Intermediate 50: (S)-9-(4-((1R,2S)-6-(tert-butoxy)-2-phenyl-1,2,3,4-tetrahydronaphthalen-1-yl)phenyl)-3-(dimethoxymethyl)-1-oxa-9-azaspiro[5.5]undecane

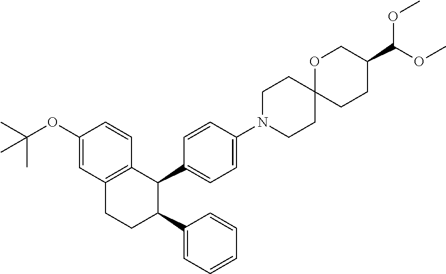 [SMILES: COC(OC)[C@H]1CCC2(CCN(c3ccc([C@@H]4c5ccc(OC(C)(C)C)cc5CC[C@@H]4c4ccccc4)cc3)CC2)OC1]

To a solution of 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutane-1-sulfonate (3.50 g, 1 Eq, 5.35 mmol), (S)-3-(dimethoxymethyl)-1-oxa-9-azaspiro[5.5]undecane (1.59 g, 1.3 Eq, 6.95 mmol), Xphos (255 mg, 0.1 Eq, 535 μmol) and Cs₂CO₃ (3,48 g, 2 Eq, 10.7 mmol) in dioxane (35 mL) was added Pd₂(dba) 3 (490 mg, 0.1 Eq, 535 μmol). The mixture was stirred at 100° C. for 24 hours. TLC (Petroleum ether: Ethyl acetate=5:1, R_{f}=0.68) indicated 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutanc-1-sulfonate was consumed completely, and one new spot formed. The reaction mixture was filtered and concentrated under reduced pressure to give a residue. The residue was purified by silica gel chromatography (column height: 250 mm, diameter: 100 mm, 100-200 mesh silica gel, Petroleum ether/Ethyl acetate=100/1, 1/1). (S)-9-(4-((1R,2S)-6-(tert-butoxy)-2-phenyl-1,2,3,4-tetrahydronaphthalen-1-yl)phenyl)-3-(dimethoxymethyl)-1-oxa-9-azaspiro[5.5]undecane (2.00 g, 3.35 mmol, 62.7% yield) was obtained as a yellow solid, ¹H NMR (400 MHZ, CHLOROFORM-d) δ =7.20-7.11 (m, 3H), 6.88-6.78 (m, 4H), 6.73 (dd, J=2,3, 8.3 Hz, 1H), 6.58 (d, J=8.7 Hz, 2H), 6.27 (d, J=8.6 Hz, 2H), 4.23 (br d, J=4.8 Hz, 1H), 4.17 (d, J=7.5 Hz, 1H), 3.72 (br dd, J=4.0, 11.7 Hz, 1H), 3.44 (dd, J=10.0, 11.8 Hz, 1H), 3.34 (d, J=1.3 Hz, 7H), 3.25-3.13 (m, 2H), 3.09-2.99 (m, 3H), 2.93 (dt, J=2.7, 11.6 Hz, 1H), 2.26-2.08 (m, 2H), 1.98-1.86 (m, 1H), 1.85-1.76 (m, 1H), 1.76-1.64 (m, 3H), 1.59 (br d, J=4.8 Hz, 1H), 1.55-1.41 (m, 4H), 1.37 (s, 9H), m/z+1=583.8.

### Compound A8. (3R)-3-((4aR)-3-((2-(4-((1S,2R)-6-hydroxy-2-phenyl-1,2,3,4-tetrahydronaphthalen-1-yl)phenyl)-5-oxa-2-azaspiro[3,4]octan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

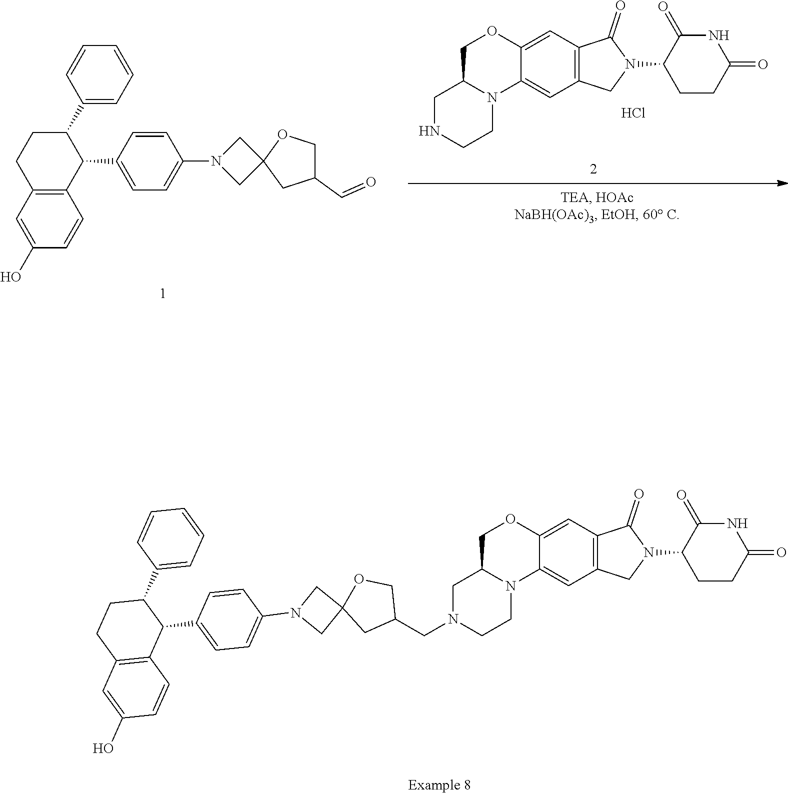 [SMILES: O=CC1COC2(C1)CN(c1ccc([C@@H]3c4ccc(O)cc4CC[C@@H]3c3ccccc3)cc1)C2 ; O=C1CC[C@H](N2Cc3cc4c(cc3C2=O)OC[C@@H]2CNCCN42)C(=O)N1 ; Cl ; O=C1CC[C@H](N2Cc3cc4c(cc3C2=O)OC[C@@H]2CN(CC3COC5(C3)CN(c3ccc([C@@H]6c7ccc(O)cc7CC[C@@H]6c6ccccc6)cc3)C5)CCN42)C(=O)N1]

To a mixture of 2-(4-((1R,2S)-6-hydroxy-2-phenyl-1,2,3,4-tetrahydronaphthalen-1-yl)phenyl)-5-oxa-2-azaspiro[3,4]octane-7-carbaldehyde (30 mg, 0.07 mmol, 1.0 eq) and rac-(R)-3-((R)-8-oxo-1,2,3,4,4a,5,8,10-octahydro-9H-pyrazino[1′,2′: 4,5][1,4]oxazino[2,3-f]isoindol-9-yl)piperidine-2,6-dione hydrochloride (27 mg, 0.07 mmol, 1.0 eq) in EtOH (5 mL) was added triethylamine (27.5 mg, 0.27 mmol, 4.0 eq), followed by the addition of AcOH (163 mg, 2.72 mmol, 40 eq) and stirred at 25° C. for 0.5 hour. The mixture was added NaBH (OAc) 3 (58 mg, 0.27 mmol, 4.0 eq) and stirred at 50° C. for 2 hours. LCMS showed the reaction was completed. The reaction was cooled to 20° C. and concentrated under vacuum. The residue was purified by Prep-HPLC(ACN/0.05% FA) to afford (3R)-3-((4aR)-3-((2-(4-((1S,2R)-6-hydroxy-2-phenyl-1,2,3,4-tetrahydronaphthalen-1-yl)phenyl)-5-oxa-2-azaspiro[3,4]octan-7-yl)methyl)-8-oxo-1,2,3,4,4a,5,8,10-octahydro-9H-pyrazino[11,2′: 4,5][1,4]oxazino[2,3-f]isoindol-9-yl)piperidine-2,6-dione (5.77 mg, 19%) as a white solid. LC-MS purity: 100% (UV at 254 nm), 780.2 [M+H]⁺. ¹H NMR (400 MHZ, DMSO) δ 10.94 (s, 1H), 9.12 (s, 1H), 7.19-7.07 (m, 4H), 7.04-6.95 (m, 2H), 6.80 (dd, J=16.7, 7.5 Hz, 2H), 6.66-6.57 (m, 2H), 6.47 (dd, J=8.3, 2.6 Hz, 1H), 6.31 (d, J=8.3 Hz, 1H), 6.18 (d, J=8.4 Hz, 1H), 6.06 (d, J=8.3 Hz, 1H), 5.03 (dd, J=13.1, 4.9 Hz, 1H), 4.31 (d, J=9.6 Hz, 1H), 4.21 (d, J=22.7 Hz, 2H), 4.15-4.10 (m, 1H), 4.00-3.87 (m, 3H), 3.87-3.71 (m, 3H), 3.70-3.60 (m, 2H), 3.59-3.49 (m, 3H), 3.03-2,76 (m, 6H), 2.71-2.58 (m, 3H), 2.44-2.29 (m, 3H), 2.09 (dd, J=14.0, 8.4 Hz, 1H), 2.01-1.82 (m, 3H), 1.69 (d, J=11.3, 8 Hz, 1H),

### Compound A21. (S)-3-((S)-3-((1-(4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

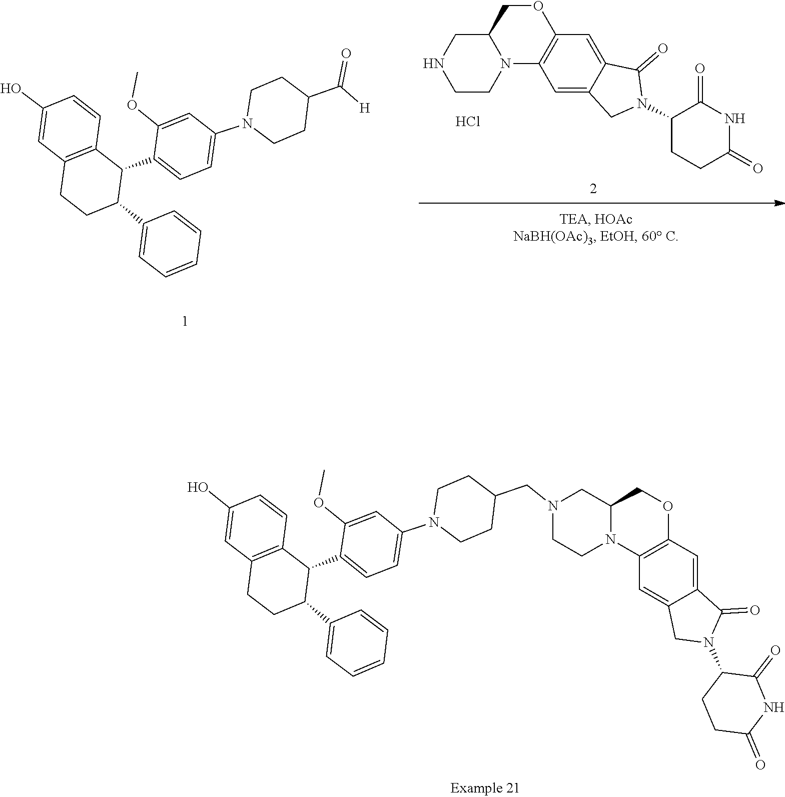 [SMILES: COc1cc(N2CCC(C=O)CC2)ccc1[C@H]1c2ccc(O)cc2CC[C@H]1c1ccccc1 ; O=C1CC[C@H](N2Cc3cc4c(cc3C2=O)OC[C@@H]2CNCCN42)C(=O)N1 ; Cl ; COc1cc(N2CCC(CN3CCN4c5cc6c(cc5OC[C@@H]4C3)C(=O)N([C@H]3CCC(=O)NC3=O)C6)CC2)ccc1[C@H]1c2ccc(O)cc2CC[C@H]1c1ccccc1]

To a mixture of 1-(4-((1R,2R)-6-hydroxy-2-phenyl-1,2,3,4-tetrahydronaphthalen-1-yl)-3-methoxyphenyl)piperidine-4-carbaldehyde (45.4 mg, 0.10 mmol, 1.0 eq) and(S)-3-((S)-8-oxo-1,2,3,4,4a,5,8,10-octahydro-9H-pyrazino[11,2′: 4,5][1,4]oxazino[2,3-f]isoindol-9-yl)piperidine-2,6-dione hydrochloride (40.4 mg, 0.10 mmol, 1.0 eq) in EtOH (5 mL) was added triethylamine (0.06 mL, 0.41 mmol, 4.0 eq), followed by the addition of AcOH (0.32 mL, 4.11 mmol, 40 eq) and stirred at 25° C. for 0.5 hour. The mixture was added NaBH (OAc) 3 (87.2 mg, 0.41 mmol, 4.0 eq) and stirred at 60° C. for 2 hours. LCMS showed the reaction was completed. The reaction was cooled to 20° C. and concentrated under vacuum. The residue was purified by prep-HPLC(ACN/2 afford 0.05% FA) (S)-3-((S)-3-((1-(4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (37.96 mg,47%) as a white solid. LCMS purity: 100% (UV at 254 nm), MS: 782.2 [M+H]⁺; Retention time R_{f}=5.256 min, ¹H NMR (400M Hz, MeOD-d₄) δ 7.15 (s, 1H), 7.07 (dd, J=7.4, 4.0 Hz, 4H), 6.80-6.73 (m, 2H), 6.67-6.61 (m, 2H), 6.57 (d, J=8.3 Hz, 1H), 6.51 (dd, J=8.3, 2.6 Hz, 2H), 6.28 (s, 1H), 5.51 (s, 1H), 5.10 (dd, J=13.3, 5.2 Hz, 1H), 4.81 (d, J=5.3 Hz, 1H), 4.41-4,27 (m, 3H), 4.03 (dd, J=11.0, 7.9 Hz, 2H), 3.62 (d, J=9.2 Hz, 2H), 3.46 (dd, J=17.5, 12.1 Hz, 2H), 3.26 (d, J=7.3 Hz, 2H), 3.05 (s, 4H), 2.94 (ddd, J=18.3, 15.7, 5.4 Hz, 3H), 2.80 (dd, J=11.9, 7.6 Hz, 3H), 2.68 (s, 2H), 2.53-2.41 (m, 1H), 2.38-2.25 (m, 2H), 2.16 (dd, J=11,2, 6.2 Hz, 1H), 1.95 (t, J=10.1 Hz, 3H), 1.68 (dd, J=15.3, 5.5 Hz, 1H), 1.52-1.42 (m, 2H),

### Compound A25. (S)-3-(1′-(((R)-8-(4-((1S,2R)-6-hydroxy-2-phenyl-1,2,3,4-tetrahydronaphthalen-1-yl)phenyl)-1-oxa-8-azaspiro[4.5]decan-3-yl)methyl)-7-oxo-5,7-dihydro-2H,6H-spiro[furo[2,3-f]isoindole-3,4′-piperidin]-6-yl)piperidine-2,6-dione

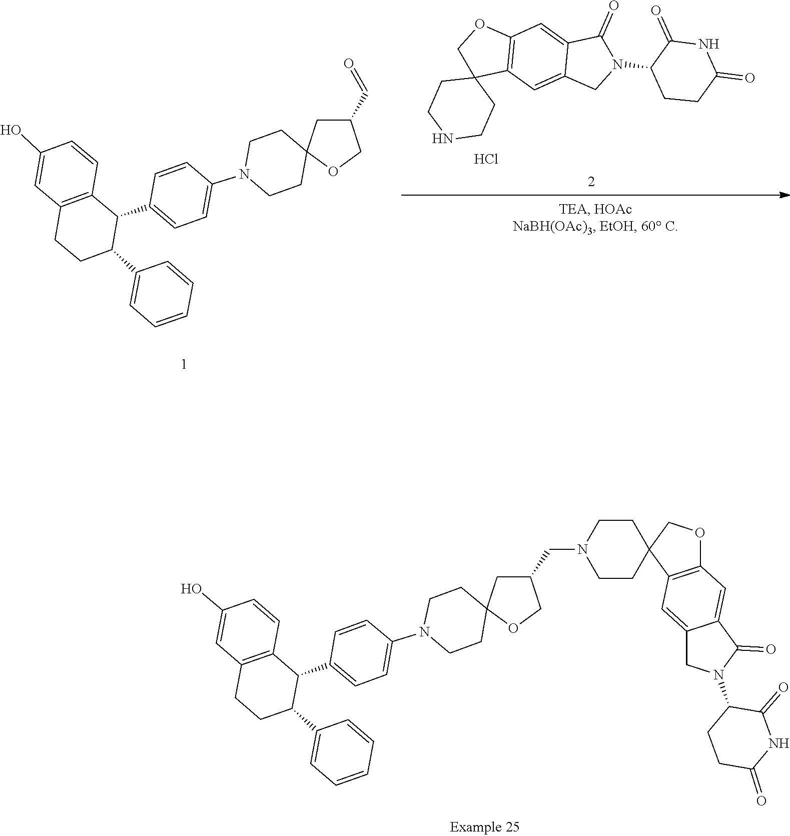 [SMILES: O=C[C@@H]1COC2(CCN(c3ccc([C@H]4c5ccc(O)cc5CC[C@H]4c4ccccc4)cc3)CC2)C1 ; O=C1CC[C@H](N2Cc3cc4c(cc3C2=O)OCC42CCNCC2)C(=O)N1 ; Cl ; O=C1CC[C@H](N2Cc3cc4c(cc3C2=O)OCC42CCN(C[C@@H]3COC4(CCN(c5ccc([C@H]6c7ccc(O)cc7CC[C@H]6c6ccccc6)cc5)CC4)C3)CC2)C(=O)N1]

To a mixture of(S)-8-(4-((1S,2R)-6-hydroxy-2-phenyl-1,2,3,4-tetrahydronaphthalen-1-yl)phenyl)-1-oxa-8-azaspiro[4.5]decane-3-carbaldehyde (40 mg, 0.09 mmol, 1.0 eq) and rac-(R)-3-(7-oxo-5,7-dihydro-2H,6H-spiro[furo[2,3-f]isoindole-3,4′-piperidin]-6-yl)piperidine-2,6-dione hydrochloride (32 mg, 0.09 mmol, 1.0 eq) in EtOH (5 mL) was added triethylamine (37 mg, 0.36 mmol, 4.0 eq), followed by the addition of AcOH (219 mg, 3.6 mmol, 40 eq) and stirred at 25° C. for 0.5 hour. The mixture was added NaBH (OAc) 3 (77 mg, 0.36 mmol, 4.0 eq) and stirred at 50° C. for 2 hours. LCMS showed the reaction was completed. The reaction was cooled to 20° C. and concentrated under vacuum. The residue was purified by prep-HPLC(ACN/0.05% FA) to afford (3S)-3-(1′-((8-(4-((1S,2R)-6-hydroxy-2-phenyl-1,2,3,4-tetrahydronaphthalen-1-yl)phenyl)-1-oxa-8-azaspiro[4.5]decan-3-yl)methyl)-7-oxo-5,7-dihydro-2H,6H-spiro[furo[2,3-f]isoindole-3,4′-piperidin]-6-yl)piperidine-2,6-dione (4.9 mg,12%) as a white solid. LC-MS purity: 100% (UV at 254 nm), 807.2 [M+H]⁺. ¹H NMR (400 MHZ, DMSO) δ 10.96 (s, 1H), 9.13 (s, 1H), 8.19 (s, 1H), 7.46 (s, 1H), 7.13 (t, J=7.6 Hz, 3H), 7.00 (s, 1H), 6.82 (d, J=6.7 Hz, 2H), 6.65 (d, J=8.3 Hz, 1H), 6.59 (s, 1H), 6.53 (d, J=8.5 Hz, 2H), 6.48 (d, J=8.2 Hz, 1H), 6.20 (d, J=8.5 Hz, 2H), 5.07 (dd, J=13.0, 5.2 Hz, 1H), 4.45 (s, 2H), 4.33 (d, J=16.9 Hz, 1H), 4.20 (d, J=17.0 Hz, 1H), 4.12 (d, J=4.7 Hz, 1H), 3.86 (t, J=7.6 Hz, 1H), 3.46-3.41 (m, 3H), 3.02 (s, 4H), 2.98-2,78 (m, 6H), 2.64 (d, J=26.2 Hz, 1H), 2.34 (t, J=16.7 Hz, 3H), 2.17-2.05 (m, 1H), 1.93 (ddd, J=22.8, 14.6, 8.6 Hz, 6H), 1.71-1.57 (m, 6H), 1.33 (dd, J=12.6, 7.2 Hz, 1H),

### Compound A34. (R)-3-((R)-3-((1-(4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

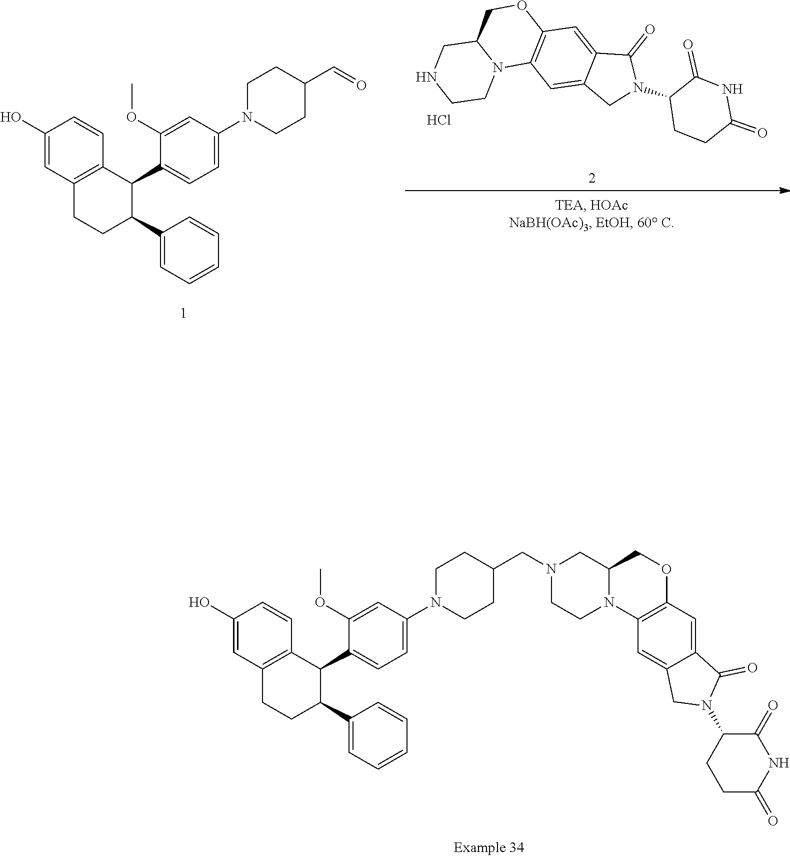 [SMILES: COc1cc(N2CCC(C=O)CC2)ccc1[C@@H]1c2ccc(O)cc2CC[C@@H]1c1ccccc1 ; O=C1CC[C@H](N2Cc3cc4c(cc3C2=O)OC[C@@H]2CNCCN42)C(=O)N1 ; Cl ; COc1cc(N2CCC(CN3CCN4c5cc6c(cc5OC[C@@H]4C3)C(=O)N([C@H]3CCC(=O)NC3=O)C6)CC2)ccc1[C@@H]1c2ccc(O)cc2CC[C@@H]1c1ccccc1]

To a mixture of 1-(4-((1R,2R)-6-hydroxy-2-phenyl-1,2,3,4-tetrahydronaphthalen-1-yl)-3-methoxyphenyl)piperidine-4-carbaldehyde with structure being tentatively assigned (40 mg, 0.09 mmol, 1.0 eq) and (R)-3-((R)-8-oxo-1,2,3,4,4a,5,8,10-octahydro-9H-pyrazino[1′,2′: 4,5][1,4]oxazino[2,3-f]isoindol-9-yl)piperidine-2,6-dione hydrochloride (32 mg, 0.09 mmol, 1.0 eq) in EtOH (5 mL) was added triethylamine (37 mg, 0.36 mmol, 4.0 eq), followed by the addition of AcOH (219 mg, 3.64 mmol, 40 eq) and stirred at 25° C. for 0.5 hour. The mixture was added NaBH (OAc) 3 (77 mg, 0.36 mmol, 4.0 eq) and stirred at 50° C. for 2 hours. LCMS showed the reaction was completed. The reaction was cooled to 20° C. and concentrated under vacuum. The residue was purified by Prep-HPLC(ACN/0.05% FA) to afford (R)-3-((R)-3-((1-(4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (11,29 mg, 28%) as a white solid. LC-MS purity: 100% (UV at 254 nm), 782.2 [M+H]⁺, ¹H NMR (400 MHZ, DMSO) δ 10.94 (s, 1H), 9.06 (s, 1H), 7.22-6.92 (m, 5H), 6.73 (d, J=5.8 Hz, 2H), 6.60-6.51 (m, 2H), 6.49-6.42 (m, 1H), 6.40-6.28 (m, 2H), 6.13 (t, J=6.0 Hz, 1H), 5.03 (d, J=13.0 Hz, 1H), 4.65 (d, J=5.2 Hz, 1H), 4.28-4.16 (m, 2H), 4.03-3.85 (m, 1H), 3.61 (dd, J=21.7, 12.2 Hz, 3H), 3.15 (d, J=35.1 Hz, 4H), 3.00-2.85 (m, 8H), 2.63 (d, J=29.1 Hz, 2H), 2.33 (s, 1H), 2.23-2.10 (m, 3H), 1.96 (dd, J=10.7, 5.9 Hz, 2H), 1.85-1.73 (m, 3H), 1.58 (dd, J=11,1, 8.2 Hz, 3H), 1.24 (s, 2H),

### Compound A35. (S)-3-((S)-7-((1-(2-fluoro-4-((1S,2S)-6-hydroxy-2-phenyl-1,2,3,4-tetrahydronaphthalen-1-yl)-5-methoxyphenyl)piperidin-4-yl)methyl)-1-oxo-1,3,5,5a,6,7,8,9-oCtahydro-2H-pyrazino[1′,2′: 4,5][1,4]oxazino[2,3-e]isoindol-2-yl)piperidine-2,6-dione

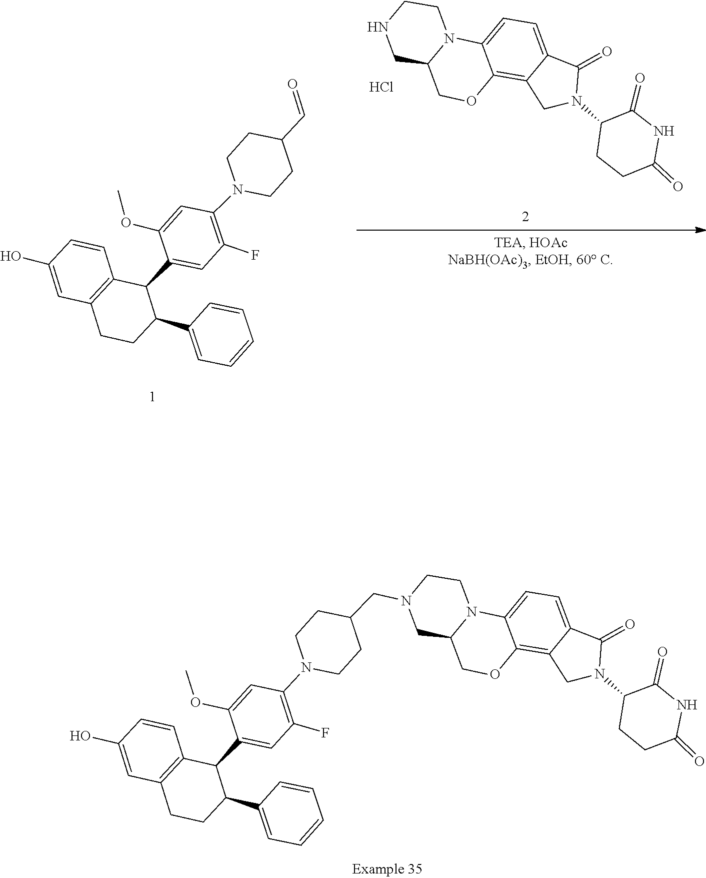 [SMILES: COc1cc(N2CCC(C=O)CC2)c(F)cc1[C@@H]1c2ccc(O)cc2CC[C@@H]1c1ccccc1 ; Cl ; O=C1CC[C@H](N2Cc3c(ccc4c3OC[C@H]3CNCCN43)C2=O)C(=O)N1 ; COc1cc(N2CCC(CN3CCN4c5ccc6c(c5OC[C@H]4C3)CN([C@H]3CCC(=O)NC3=O)C6=O)CC2)c(F)cc1[C@@H]1c2ccc(O)cc2CC[C@@H]1c1ccccc1]

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (19 mg, 0.05 mmol, 1.0 eq) and rac-(R)-3-((S)-1-oxo-1,3,5,5a,6,7,8,9-octahydro-2H-pyrazino[11,2′: 4,5][1,4]oxazino[2,3-e]isoindol-2-yl)piperidine-2,6-dione hydrochloride (15 mg, 0.05 mmol, 1.0 eq) in EtOH (5 mL) was added triethylamine (26 mg, 0.2 mmol, 4.0 eq), followed by the addition of AcOH (150 mg, 2 mmol, 40 eq) and stirred at 25° C. for 0.5 hour. The mixture was added NaBH (OAc) 3 (59 mg, 0.2 mmol, 4.0 eq) and stirred at 50° C. for 2 hours. LCMS showed the reaction was completed. The reaction was cooled to 20° C. and concentrated under vacuum. The residue was purified by prep-HPLC(ACN/0.05% FA) to afford (S)-3-((R)-7-((1-(2-fluoro-4-((1S,2S)-6-hydroxy-2-phenyl-1,2,3,4-tetrahydronaphthalen-1-yl)-5-methoxyphenyl)piperidin-4-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (5.79 mg,30%) as a white solid. LC-MS purity: 100% (UV at 254 nm), 800.1 [M+H]⁺. ¹H NMR (400 MHz, MeOD) δ 7.32 (d, J=8.3 Hz, 1H), 7.09 (d, J=7.1 Hz, 3H), 7.03 (d, J=8.5 Hz, 1H), 6.81-6.76 (m, 2H), 6.65 (dd, J=8.2, 5.4 Hz, 2H), 6.54 (dd, J=8.4, 2.4 Hz, 1H), 6.29 (d, J=13.6 Hz, 1H), 6.24 (d, J=7.5 Hz, 1H), 5.10 (dd, J=13.3, 5.0 Hz, 1H), 4.80 (d, J=5.0 Hz, 1H), 4.41-4,27 (m, 3H), 4.10-4.01 (m, 1H), 3.86 (d, J=12.1 Hz, 1H), 3.19-3.05 (m, 2H), 3.01 (d, J=22.5 Hz, 6H), 2.89 (dd, J=13.1, 5.4 Hz, 2H), 2.82-2,75 (m, 1H), 2.72-2.58 (m, 2H), 2.50 (dd, J=13.2, 4.7 Hz, 1H), 2.35 (d, J=7.0 Hz, 2H), 2.28 (dd, J=22,3, 10.0 Hz, 2H), 2.20-2.11 (m, 1H), 1.88 (dd, J=7.6, 3.8 Hz, 3H), 1.73 (dt, J=14.3, 7.0 Hz, 2H), 1.38 (ddd, J=25.5, 18.7, 13.9 Hz, 5H).

### Compound A52. (3S)-3-((5aR)-7-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

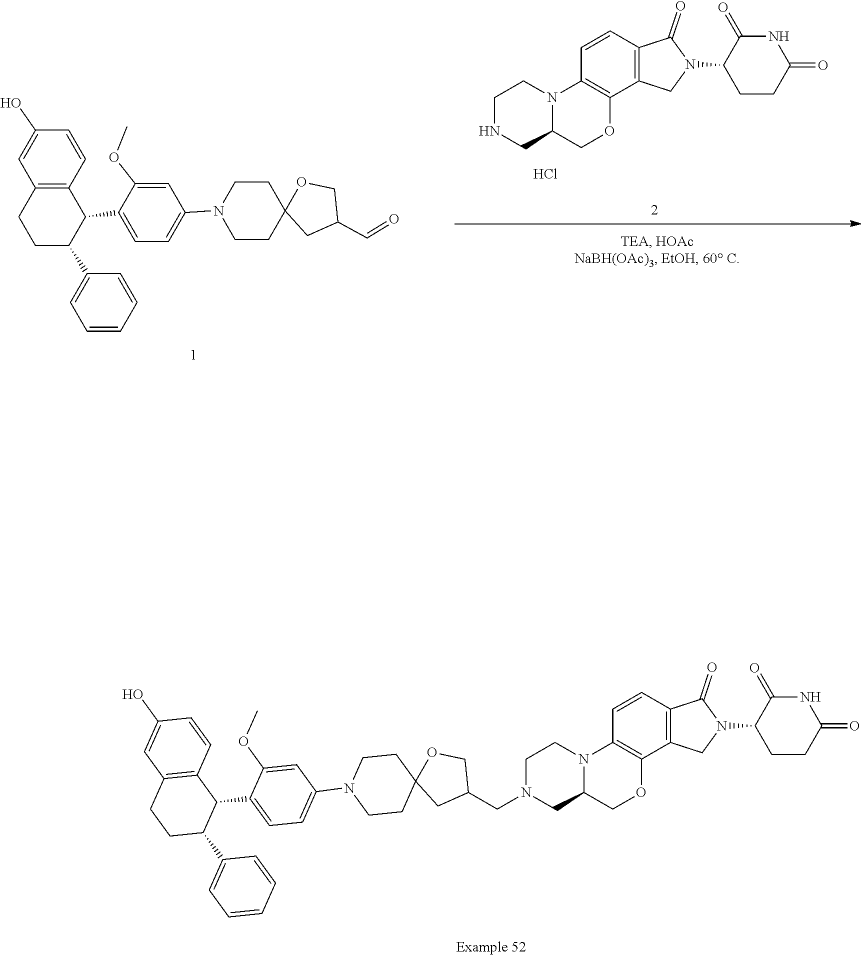 [SMILES: COc1cc(N2CCC3(CC2)CC(C=O)CO3)ccc1[C@H]1c2ccc(O)cc2CC[C@H]1c1ccccc1 ; O=C1CC[C@H](N2Cc3c(ccc4c3OC[C@H]3CNCCN43)C2=O)C(=O)N1 ; Cl ; COc1cc(N2CCC3(CC2)CC(CN2CCN4c5ccc6c(c5OC[C@H]4C2)CN([C@H]2CCC(=O)NC2=O)C6=O)CO3)ccc1[C@H]1c2ccc(O)cc2CC[C@H]1c1ccccc1]

To a mixture of 8-(4-((1R,2R)-6-hydroxy-2-phenyl-1,2,3,4-tetrahydronaphthalen-1-yl)-3-methoxyphenyl)-1-oxa-8-azaspiro[4.5]decane-3-carbaldehyde (60 mg, 0.14 mmol, 1.0 eq) and rac-(R)-3-((S)-1-oxo-1,3,5,5a,6,7,8,9-octahydro-2H-pyrazino[1′,2′: 4,5][1,4]oxazino[2,3-e]isoindol-2-yl)piperidine-2,6-dione (48.3 mg, 0.14 mmol, 1.0 eq) in EtOH (5 mL) was added triethylamine (55 mg, 0.54 mmol, 4.0 eq), followed by the addition of AcOH (327 mg, 5.4 mmol, 40 eq) and stirred at 25° C. for 0.5 hour. The mixture was added NaBH (OAc) 3 (115 mg, 0.54 mmol, 4.0 eq) and stirred at 50° C. for 2 hours. LCMS showed the reaction was completed. The reaction was cooled to 20° C. and concentrated under vacuum. The residue was purified by prep-HPLC(ACN/0.05% FA) to afford (3S)-3-((5aR)-7-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-1-oxo-1,3,5,5a,6,7,8,9-octahydro-2H-pyrazino[1′0.2′: 4,5][1,4]oxazino[2,3-e]isoindol-2-yl)piperidine-2,6-dione (18 mg, 13%) as a white solid. LC-MS purity: 100% (UV at 254 nm), 800.1 [M+H]⁺, 1H NMR (400 MHZ, MeOD) δ 7.31 (d, J=8.3 Hz, 1H), 7.05 (dt, J=12.0, 6.0 Hz, 4H), 6.78-6.72 (m, 2H), 6.64 (d, J=8.3 Hz, 2H), 6.54-6.48 (m, 2H), 6.43 (dd, J=8.5, 2.1 Hz, 1H), 6.21 (d, J=2.1 Hz, 1H), 5.10 (dd, J=13.2, 5.1 Hz, 1H), 4.78 (d, J=5.3 Hz, 1H), 4.40-4.34 (m, 2H), 4.05 (dd, J=12.5, 6.7 Hz, 2H), 3.86 (d, J=11.8 Hz, 1H), 3.61 (t, J=8.0 Hz, 1H), 3.23 (s, 1H), 3.15 (s, 3H), 3.03 (s, 3H), 3.00 (d, J=5.5 Hz, 2H), 2.89 (dd, J=12.8, 4.9 Hz, 2H), 2.82-2,76 (m, 1H), 2.74-2.66 (m, 1H), 2.48 (d, J=7.2 Hz, 2H), 2.30 (s, 1H), 2.20-2.05 (m, 3H), 1.89 (d, J=18.7 Hz, 1H), 1.80 (s, 4H), 1.65 (s, 2H), 1.49 (dd, J=12.6, 8.3 Hz, 1H), 1.33 (d, J=18.0 Hz, 5H).

### Compound A54. (3S)-3-(1′-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-6-oxo-6,8-dihydro-2H,7H-spiro[furo[2,3-e]isoindole-3,4′-piperidin]-7-yl)piperidine-2,6-dione

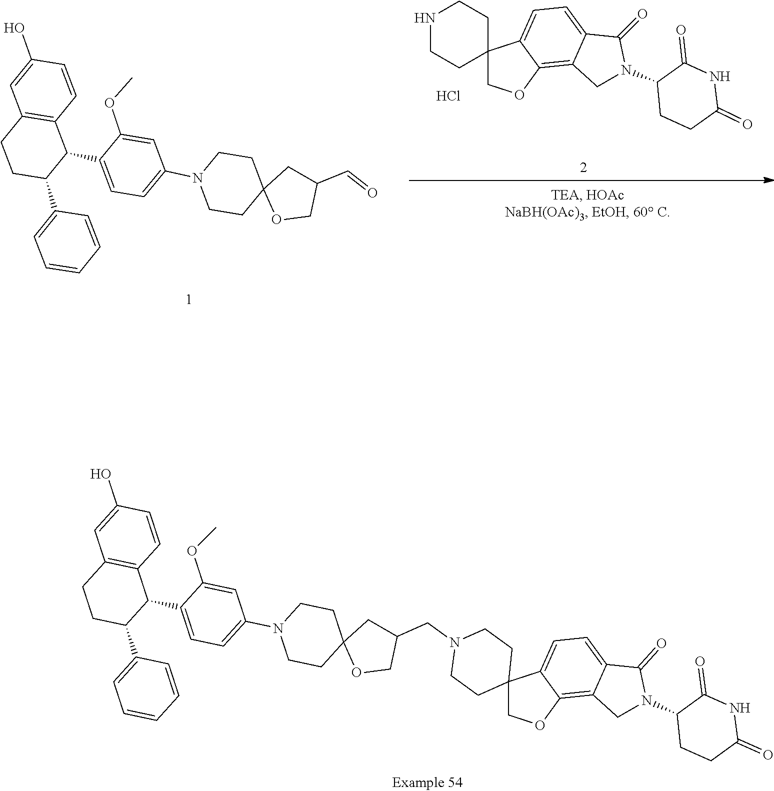 [SMILES: COc1cc(N2CCC3(CC2)CC(C=O)CO3)ccc1[C@H]1c2ccc(O)cc2CC[C@H]1c1ccccc1 ; O=C1CC[C@H](N2Cc3c(ccc4c3OCC43CCNCC3)C2=O)C(=O)N1 ; Cl ; COc1cc(N2CCC3(CC2)CC(CN2CCC4(CC2)COc2c4ccc4c2CN([C@H]2CCC(=O)NC2=O)C4=O)CO3)ccc1[C@H]1c2ccc(O)cc2CC[C@H]1c1ccccc1]

To a mixture of 8-(4-((1R,2R)-6-hydroxy-2-phenyl-1,2,3,4-tetrahydronaphthalen-1-yl)-3-methoxyphenyl)-1-oxa-8-azaspiro[4.5]decane-3-carbaldehyde (30 mg, 0.06 mmol, 1.0 eq) and (R)-3-(6-oxo-6,8-dihydro-2H,7H-spiro[furo[2,3-e]isoindole-3,4′-piperidin]-7-yl)piperidine-2,6-dione hydrochloride (23 mg, 0.06 mmol, 1.0 eq) in EtOH (5 mL) was added triethylamine (26 mg, 0.26 mmol, 4.0 eq), followed by the addition of AcOH (154 mg, 2.56 mmol, 40 eq) and stirred at 25° C. for 0.5 hour. The mixture was added NaBH (OAc) 3 (54.3 mg, 0.26 mmol, 4.0 eq) and stirred at 50° C. for 2 hours. LCMS showed the reaction was completed. The reaction was cooled to 20° C. and concentrated under vacuum. The residue was purified by Prep-HPLC(ACN/0.05% FA) to afford (3S)-3-(1′-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-6-oxo-6,8-dihydro-2H,7H-spiro[furo[2,3-e]isoindole-3,4′-piperidin]-7-yl)piperidine-2,6-dione (8.4 mg,28%) as a white solid. LC-MS purity: 100% (UV at 254 nm), 837.6 [M+H]⁺. ¹H NMR (400 MHz, DMSO) δ 10.97 (s, 1H), 9.05 (s, 1H), 7.40 (d, J=7.6 Hz, 1H), 7.26 (d, J=7.6 Hz, 1H), 7.07 (d, J=6.6 Hz, 3H), 6.72 (d, J=9.1 Hz, 2H), 6.56 (d, J=8.5 Hz, 2H), 6.45 (d, J=10.7 Hz, 1H), 6.37 (d, J=8.5 Hz, 1H), 6.31 (d, J=6.3 Hz, 1H), 6.11 (s, 1H), 5.08 (dd, J=12.0, 4.2 Hz, 1H), 4.64 (d, J=5.3 Hz, 1H), 4.52 (s, 2H), 4.37 (d, J=17.0 Hz, 1H), 4.21 (d, J=16.9 Hz, 1H), 3.88 (t, J=7.8 Hz, 1H), 3.47-3.42 (m, 1H), 3.20 (dd, J=12.9, 6.6 Hz, 1H), 3.08 (s, 4H), 2.94 (s, 3H), 2.88 (dd, J=19.0, 11,1 Hz, 4H), 2.64 (d, J=25.1 Hz, 2H), 2.34 (d, J=7.0 Hz, 2H), 2.22-2.14 (m, 1H), 2.00 (d, J=15.9 Hz, 2H), 1.89 (dd, J=27.6, 14.3 Hz, 4H), 1.71-1.64 (m, 4H), 1.60 (d, J=9.6 Hz, 4H), 1.39-1.22 (m, 2H),

### Compound A56. (3S)-3-((4aS)-3-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

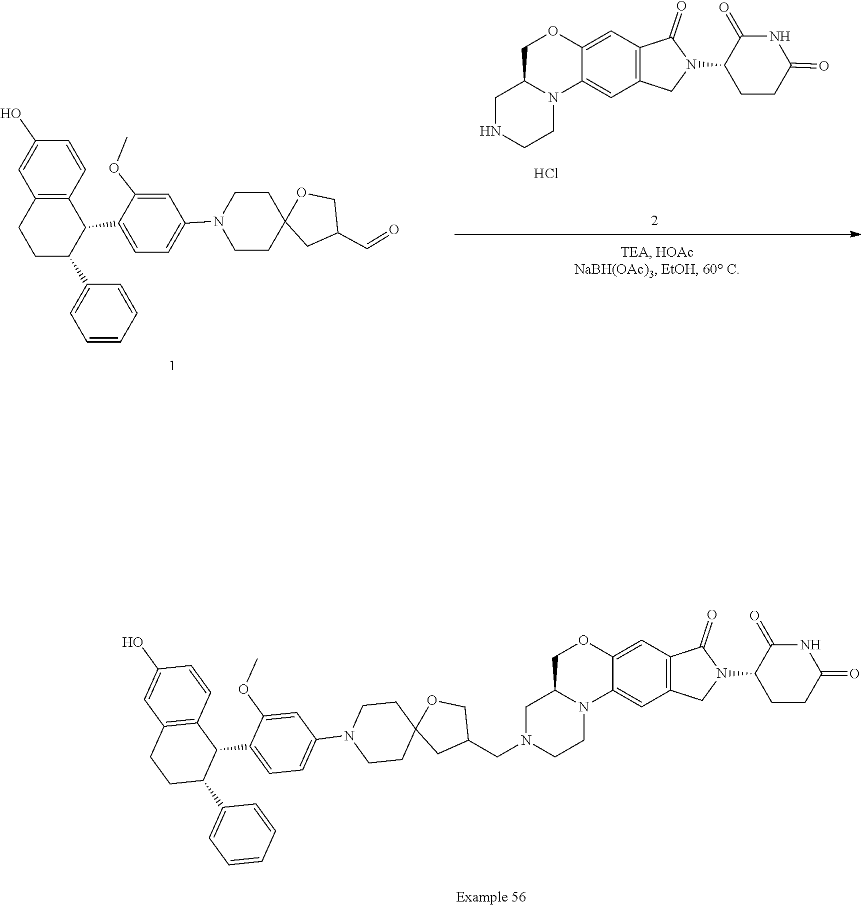 [SMILES: COc1cc(N2CCC3(CC2)CC(C=O)CO3)ccc1[C@H]1c2ccc(O)cc2CC[C@H]1c1ccccc1 ; O=C1CC[C@H](N2Cc3cc4c(cc3C2=O)OC[C@@H]2CNCCN42)C(=O)N1 ; Cl ; COc1cc(N2CCC3(CC2)CC(CN2CCN4c5cc6c(cc5OC[C@@H]4C2)C(=O)N([C@H]2CCC(=O)NC2=O)C6)CO3)ccc1[C@H]1c2ccc(O)cc2CC[C@H]1c1ccccc1]

To a mixture of 8-(4-((1R,2R)-6-hydroxy-2-phenyl-1,2,3,4-tetrahydronaphthalen-1-yl)-3-methoxyphenyl)-1-oxa-8-azaspiro[4.5]decane-3-carbaldehyde (60 mg, 0.12 mmol, 1.0 eq) and (R)-3-((R)-8-oxo-1,2,3,4,4a,5,8,10-octahydro-9H-pyrazino[1′,2′:4.5][1,4]oxazino[2,3-f]isoindol-9-yl)piperidine-2,6-dione hydrochloride (43 mg, 0.12 mmol, 1.0 eq) in EtOH (5 mL) was added triethylamine (48.6 mg, 0.48 mmol, 4.0 eq), followed by the addition of AcOH (288 mg, 4.11 mmol, 40 eq) and stirred at 25° C. for 0.5 hour. The mixture was added NaBH (OAc) 3 (102 mg, 0.48 mmol, 4.0 eq) and stirred at 50° C. for 2 hours. LCMS showed the reaction was completed. The reaction was cooled to 20° C. and concentrated under vacuum. The residue was purified by Prep-HPLC(ACN/0.05% FA) to afford (3S)-3-((4aS)-3-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (17.43 mg,29%) as a white solid. LC-MS purity: 100% (UV at 254 nm), 838.6 [M+H]⁺. ¹H NMR (400 MHZ, DMSO) δ 10.93 (s, 1H), 9.05 (s, 1H), 7.06 (t. J=5.8 Hz, 4H), 6.93 (s, 1H), 6.74-6.70 (m, 2H), 6.55 (d, J=8.2 Hz, 2H), 6.45 (dd, J=8.4, 2.5 Hz, 1H), 6.36 (d, J=8.4 Hz, 1H), 6.31 (d, J=7.9 Hz, 1H), 6.11 (s, 1H), 5.07-4.99 (m, 1H), 4.64 (d, J=4.9 Hz, 1H), 4.35-3.99 (m, 4H), 3.90 (s, 3H), 3.45 (s, 1H), 3.20 (d, J=14.6 Hz, 2H), 3.08 (s, 4H), 2.94 (s, 3H), 2.67 (d, J=1.8 Hz, 2H), 2.33 (d, J=1.8 Hz, 3H), 2.11 (d, J=28.9 Hz, 3H), 1.96 (d, J=5.5 Hz, 3H), 1.65 (s, 4H), 1.60 (s, 4H), 1.35 (s, 1H), 1.24 (s, 1H),

### Compound A74. (S)-3-((R)-7-(((S)-2-(4-((1R,2S)-6-hydroxy-2-phenyl-1,2,3,4-tetrahydronaphthalen-1-yl)phenyl)-5-oxa-2-azaspiro[3,4]octan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-Compound A75. (S)-3-((R)-7-(((R)-2-(4-((1R,2S)-6-hydroxy-2-phenyl-1,2,3,4-tetrahydronaphthalen-1-yl)phenyl)-5-oxa-2-azaspiro[3,4]octan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

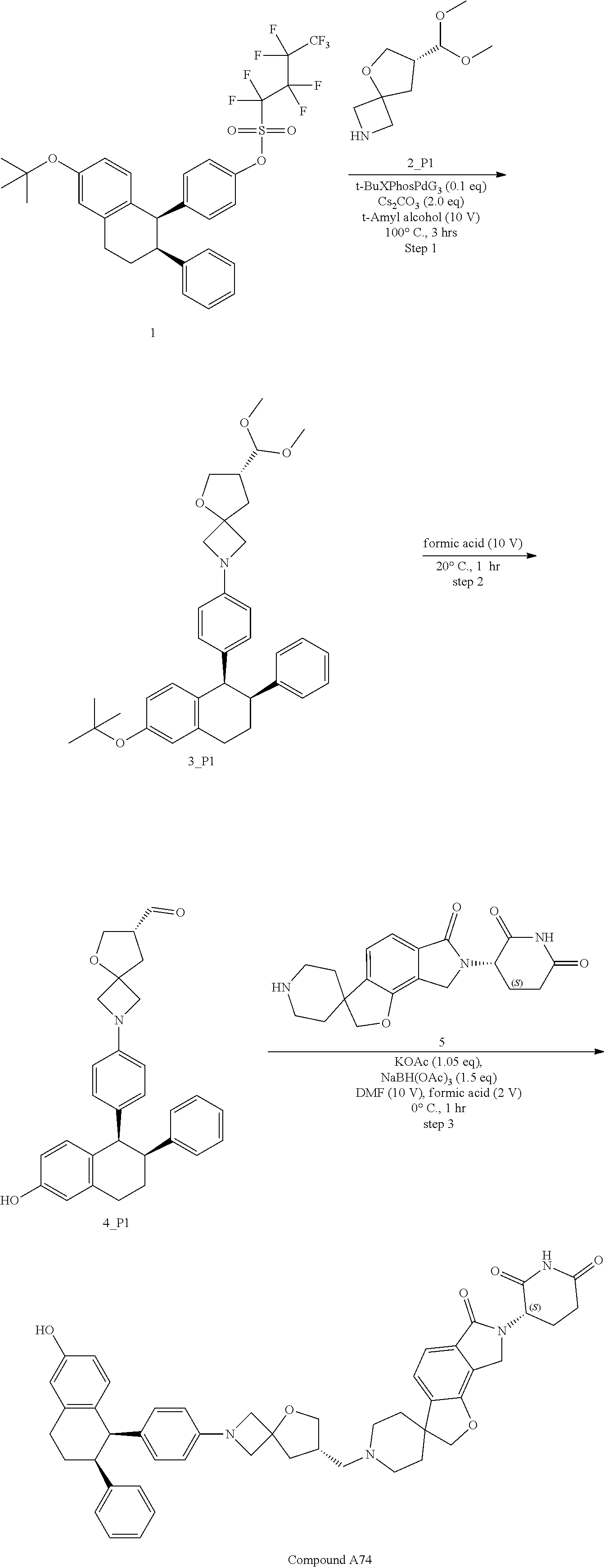

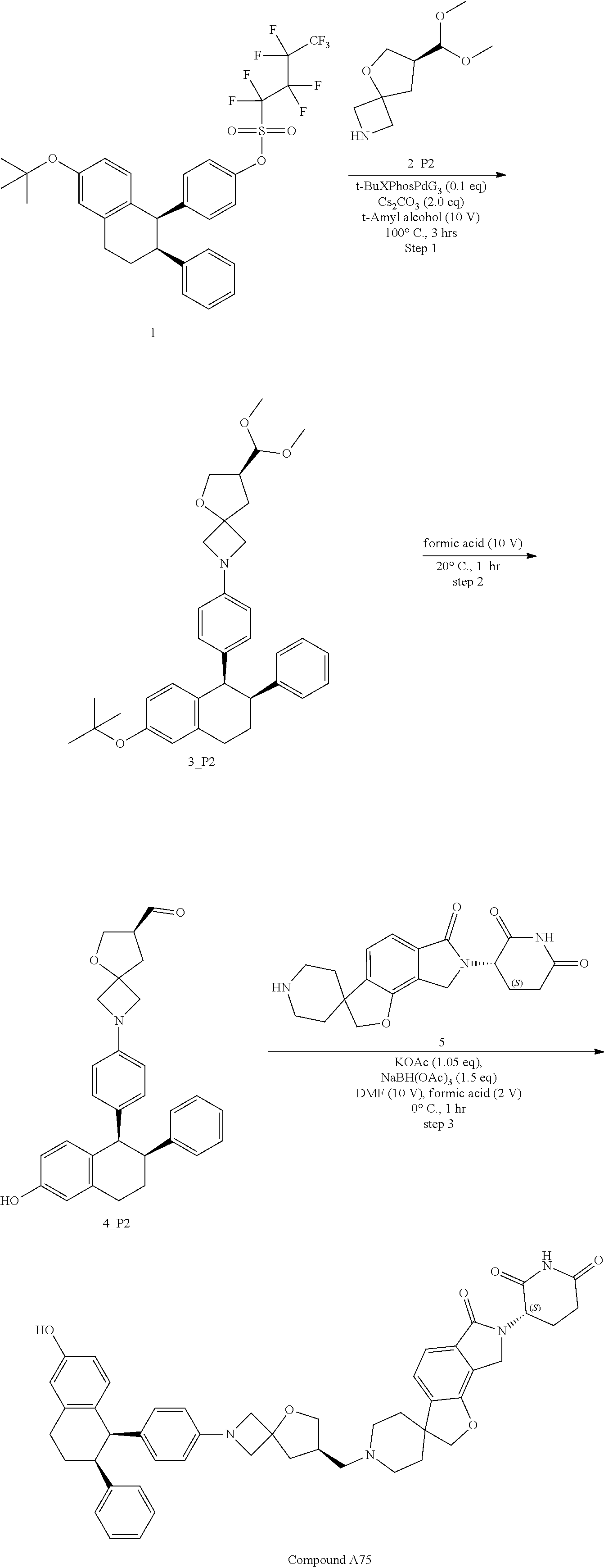

### Step 1: Synthesis of (R)-2-(4-((1R,2S)-6-(tert-butoxy)-2-phenyl-1,2,3,4-tetrahydronaphthalen-1-yl)phenyl)-7-(dimethoxymethyl)-5-oxa-2-azaspiro[3,4]octane

To the solution of compound 1 (1 g, 1.53 mmol, 1.00 eq) in 2-methyl-2-butanol (10 mL) at 20° C. was add Cs₂CO₃ (995 mg, 3.06 mmol, 2.00 eq) and compound 2_P1 (536 mg, 2.29 mmol, 80% purity, 1.50 eq), 1-BuXPhosPdG₃ (121 mg, 152 μmol, 0.10 eq) was added to the mixture at 20° C. under N₂ atmosphere. The mixture was stirred at 100° C. for 12 hrs. LCMS showed that the desired product was detected. The mixture was filtered, and the organic layer was dried over anhydrous Na₂SO₄ and concentrated under reduced pressure. The residue was purified by silica gel column chromatography (petroleum ether/EtOAc=50:1 to 5:1). The desired product compound 3_P1 (0.63 g, 1,16 mmol) was obtained as yellow oil, m/z (M+1): 542.4

### Step 2: Synthesis of (R)-2-(4-((1R,2S)-6-hydroxy-2-phenyl-1,2,3,4-tetrahydro-naphthalen-1-yl)phenyl)-5-oxa-2-azaspiro[3,4]octane-7-carbaldehyde

The solution of compound 3_P1 (0.1 g, 184 μmol, 1 eq) in formic acid (2 mL) was stirred at 20° C. for 1 hr. LCMS showed that compound 3_P1 was consumed, and the desired product was detected. The mixture was concentrated under reduced pressure. The desired product compound 4_P1 (0.08 g, 182. μmol, 98.6% yield) was obtained as a yellow oil, m/z (M+1): 440.2

### Step 3: Synthesis of(S)-3-(1′-(((S)-2-(4-((1R,2S)-6-hydroxy-2-phenyl-1,2,3,4-tetrahydronaphthalen-1-yl)phenyl)-5-oxa-2-azaspiro[3,4]octan-7-yl)methyl)-6-oxo-6,8-dihydro-2H,7H-spiro[furo[2,3-e]isoindole-3,4′-piperidin]-7-yl)piperidine-2,6-dione

To the solution of compound 5 (71.3 mg, 182 μmol, 1 eq. HCl) in DMF (2 mL) was add KOAc (18.7 mg, 191 μmol, 1.05 eq) at 20° C. After 10 min, the reaction mixture was cooled to 0° C. and NaBH (OAc) 3 (57.8 mg, 273 μmol, 1.5 eq) was added to the mixture; The solution of compound 4_P1 (0.08 g, 182 μmol, 1 eq) in formic acid (1 mL) was added at 0° C., and the mixture was stirred at 0° C. for 1 hr; LCMS showed that compound 4_P1 was consumed and the desired product was detected. The mixture was quenched with water (0.1 mL), and purified by pre-HPLC (column: Phenomenex Luna C18 75*30 mm*3 um; mobile phase: [water (FA)-ACN]; B %: 30%-60%, 8 min). The desired product Compound A74 (75,6 mg, 53.3% yield) was obtained as a white solid.

¹H NMR (400 MHZ, DMSO-d₆) 10.98 (s, 1H), 9.10 (s, 1H), 7.40 (d, J-7.5 Hz, 1H), 7.27 (d, J=7.6 Hz, 1H), 7.19-7.09 (m, 3H), 6.83 (br d, J=6.7 Hz, 2H), 6.70-6.57 (m, 2H), 6.48 (dd, J=2.4, 8.3 Hz, 1H), 6.18 (d, J=8.6 Hz, 2H), 6.06 (d, J=8.6 Hz, 2H), 5.09 (dd, J=5.1, 13.2 Hz, 1H), 4.60-4.49 (m, 2H), 4.38 (d, J=17.2 Hz, 1H), 4.22 (d, J=17.2 Hz, 1H), 4.12 (d, J=4.9 Hz, 1H), 3.88 (br t, J=7.6 Hz, 1H), 3.82-3.70 (m, 2H), 3.62 (d, J=7.6 Hz, 1H), 3.58-3.46 (m, 2H), 3.31-3,24 (m, 2H), 3.02-2.81 (m, 5H), 2.64-2.53 (m, 2H), 2.45-2.34 (m, 2H), 2.27 (br dd, J=7.4, 12.5 Hz, 1H), 2.19-1.82 (m, 7H), 1.71 (br d, J=10.6 Hz, 3H), m/z (M+1): 779.3.

### Step 4: Synthesis of(S)-2-(4-((1R,2S)-6-(tert-butoxy)-2-phenyl-1,2,3,4-tetrahydronaphthalen-1-yl)phenyl)-7-(dimethoxymethyl)-5-oxa-2-azaspiro[3,4]octane

To the solution of compound 1 (1 g, 1.53 mmol, 1.00 eq) in 2-methyl-2-butanol (10 mL) was add Cs₂CO₃ (997 mg, 3.06 mmol, 2 eq) and compound 2_P2 (537 mg, 2.30 mmol, 80% purity, 1.5 eq) at 20° C. t-BuXPhosPdG₃ (121 mg, 153 μmol, 0.1 eq) was added to the mixture at 20° C. under N₂ atmosphere, before the mixture was stirred at 100° C. for 12 hrs. LCMS showed that the desired product was detected. The mixture was filtered, and the organic layer was dried over anhydrous Na₂SO₄ and concentrated under reduced pressure. The residue was purified by silica gel column chromatography (petroleum ether:EtOAc=10:1 to 5:1). The desired product compound 3_P2 (0.62 g, 1,14 mmol, 74.8% yield) was obtained as yellow oil, m/z (M+1)=542.4

### Step 5: Synthesis of(S)-2-(4-((1R,2S)-6-hydroxy-2-phenyl-1,2,3,4-tetrahydronaphthalen-1-yl)phenyl)-5-oxa-2-azaspiro[3,4]octane-7-carbaldehyde

The solution of compound 3_P2 (0.1 g, 184 μmol, 1 eq) in formic acid (2 mL) was stirred for 1 hr at 20° C. LCMS showed that compound 3_P2 was consumed, and the desired product was detected. The mixture was concentrated under reduced pressure. The desired product compound 4 P2 (0.08 g, 98.6% yield) was obtained as a yellow oil, m/z (M+1)=440.2

### Step 6: Synthesis of(S)-3-(1′-(((R)-2-(4-((1R,2S)-6-hydroxy-2-phenyl-1,2,3,4-tetrahydronaphthalen-1-yl)phenyl)-5-oxa-2-azaspiro[3,4]octan-7-yl)methyl)-6-oxo-6,8-dihydro-2H,7H-spiro[furo[2,3-e]isoindole-3,4′-piperidin]-7-yl)piperidine-2,6-dione

To the suspension of compound 5 (71.3 mg, 182 μmol, 1 eq. HCl) in DMF (2 mL) was saturated KOAc (18.7 mg, 191 μmol, 1.05 eq) at 20° C. After 10 min, the reaction mixture was cooled to 0° C., and NaBH (OAc) 3 (57.86 mg, 273 μmol, 1.5 eq) was added to the mixture. The solution of compound 4_P2 (0.08 g, 182 μmol, 1 eq) in formic acid (1 mL) was added at 0° C., and stirred for 1 hr at 0° C. LCMS showed that compound 4_P2 was consumed and the desired product was detected. The mixture was quenched with water (0.1 mL), and purified by pre-HPLC(column: Phenomenex Luna C18 75*30 mm*3 um; mobile phase: [water (FA)-ACN]; B %: 30%- 60%, 8 min). The desired product Compound A75, (0.064 g, 44.7% yield, 99.2% purity) was obtained as a white solid.

¹H NMR (400 MHZ, DMSO-d₆) δ 10.98 (s, 1H), 9.10 (s, 1H), 7.40 (d, J=7.6 Hz, 1H), 7.27 (d, J=7.6 Hz, 1H), 7.20-7.05 (m, 3H), 6.83 (br d, J=6.8 Hz, 2H), 6.69-6.58 (m, 2H), 6.48 (dd. J=2.4, 8.3 Hz, 1H), 6.18 (d, J=8.5 Hz, 2H), 6.06 (d, J=8.6 Hz, 2H), 5.09 (dd, J=4.9, 13.4 Hz, 1H), 4.57-4.48 (m, 2H), 4.38 (d, J=17.2 Hz, 1H), 4.22 (d, J=17.2 Hz, 1H), 4.12 (br d, J=4.9 Hz, 1H), 3.87 (t. J=7.5 Hz, 1H), 3.75 (dd, J=7.7, 16.0 Hz, 2H), 3.64 (d, J=7.6 Hz, 1H), 3.58-3.45 (m, 2H), 3.26 (br s, 1H), 3.06-2.82 (m, 5H), 2.64-2.53 (m, 2H), 2.46-2.21 (m, 4H), 2.15-1.82 (m, 7H), 1.70 (br d, J=9.1 Hz, 3H), m/z (M+1)=779.3.

### Compound A91: (S)-3-((S)-3-((1-(2-fluoro-4-((1S,2S)-6-hydroxy-2-phenyl-1,2,3,4-tetrahydronaphthalen-1-yl)-5-methoxyphenyl)piperidin-4-yl)methyl)-8-oxo-3,4,4a,5-tetrahydro-1H-pyrazino[1′,2′: 4,5][1,4]oxazino[2,3-f]isoindol-9 (2H,8H,10H)-yl)piperidine-2,6-dione

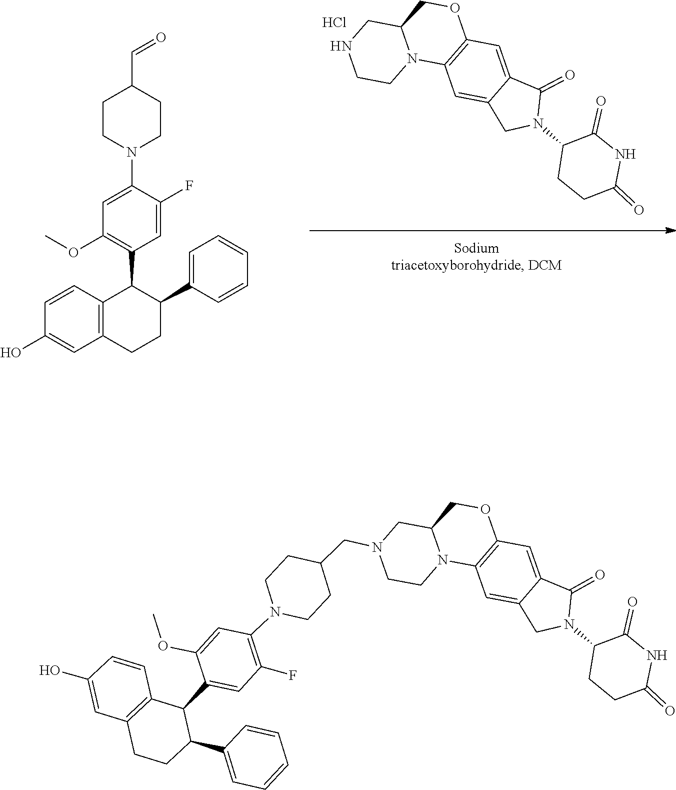 [SMILES: COc1cc(N2CCC(C=O)CC2)c(F)cc1[C@@H]1c2ccc(O)cc2CC[C@@H]1c1ccccc1 ; Cl ; O=C1CC[C@H](N2Cc3cc4c(cc3C2=O)OC[C@@H]2CNCCN42)C(=O)N1 ; COc1cc(N2CCC(CN3CCN4c5cc6c(cc5OC[C@@H]4C3)C(=O)N([C@H]3CCC(=O)NC3=O)C6)CC2)c(F)cc1[C@@H]1c2ccc(O)cc2CC[C@@H]1c1ccccc1]

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (55 mg, 0.12 mmol 1 eq.), (S)-3-((S)-8-oxo-3,4,4a,5-tetrahydro-1H-pyrazino[1′,2′: 4,5][1,4]oxazino[2,3-f]isoindol-9 (2H,8H,10H)-yl)piperidine-2,6-dione hydrochloride (70 mg, 0.17 mmol 1.5 eq.) in DCM (2 mL) was added sodium triacetoxyborohydride (51 mg, 0.24 mmol 2.0 eq.) The resultant mixture was then stirred at 25° C. for 2h. LCMS showed the reaction was completed. The reaction was cooled to 20° C. and concentrated under vacuum. The residue was purified by Prep-HPLC(acetonitril/0.05% formic acid) to afford (S)-3-((S)-3-((1-(2-fluoro-4-((1S,2S)-6-hydroxy-2-phenyl-1,2,3,4-tetrahydronaphthalen-1-yl)-5-methoxyphenyl)piperidin-4-yl)methyl)-8-oxo-3,4,4a,5-tetrahydro-1H-pyrazino[11,2′: 4,5][1,4]oxazino[2,3-f]isoindol-9 (2H,8H,10H)-yl)piperidine-2,6-dione (46.68 mg, 48.7% yield) as a white solid. LC-MS (ESI, m/z): mass calcd. For C₄₇H₅₀FN₅O₆, 799.37; found, 800.5 [M+H]⁺, ¹H NMR (400 MHZ, DMSO) δ 10.92 (s, 1H), 8.34 (s, 1H), 7.11-7.02 (m, 4H), 6.93 (s, 1H), 6.76 (d, J=7.8 Hz, 2H), 6.60-6.52 (m, 2H), 6.50-6.43 (m, 1H), 6.22-6.14 (m, 2H), 5.09-4.98 (m, 1H), 4.67 (d, J=5.0 Hz, 1H), 4.33-4.15 (m, 3H), 3.93-3.81 (m, 2H), 3.29-3,22 (m, 4H), 2.98-2.88 (m, 8H), 2.80-2,72 (m, 1H), 2.64-2.56 (m, 3H), 2.38-2.25 (m, 2H), 2.23-2.05 (m, 4H), 1.98-1.92 (m, 1H), 1.77-1.60 (m, 4H), 1.29-1.20 (m, 2H),

### Compound A93: (S)-3-(1′-((1-(2-fluoro-4-((1S,2S)-6-hydroxy-2-phenyl-1,2,3,4-tetrahydronaphthalen-1-yl)-5-methoxyphenyl)piperidin-4-yl)methyl)-6-oxo-6,8-dihydro-2H,7H-spiro[furo[2,3-e]isoindole-3,4′-piperidin]-7-yl)piperidine-2,6-dione

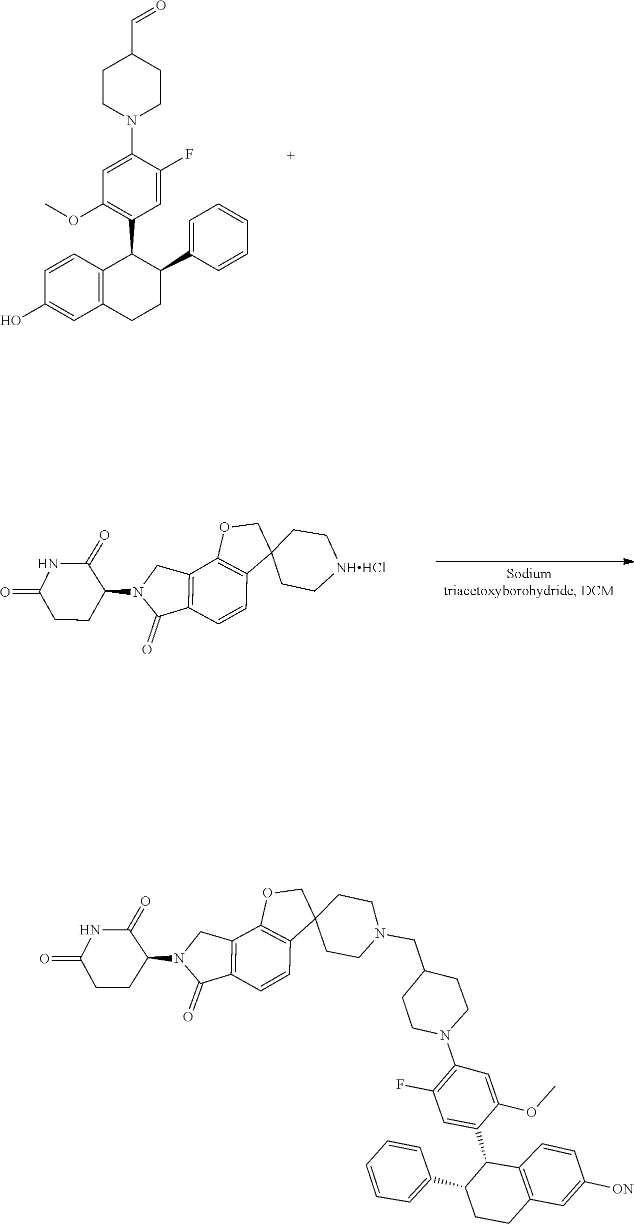

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (50.0 mg, 109 μmol, 1 eq.), (S)-3-(6-oxo-6,8-dihydro-2H,7H-spiro[furo[2,3-e]isoindole-3,4′-piperidin]-7-yl)piperidine-2,6-dione hydrochloride (61,4 mg,, 157 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (55 mg, 0.26 mmol, 2 eq.). The mixture was stirred at room temperature for 30 minutes and concentrated. The residue was by Prep-HPLC(acetonitrile/0.05% formic acid) to afford (S)-3-(1′-((1-(2-fluoro-4-((1S,2S)-6-hydroxy-2-phenyl-1,2,3,4-tetrahydronaphthalen-1-yl)-5-methoxyphenyl)piperidin-4-yl)methyl)-6-oxo-6,8-dihydro-2H,7H-spiro[furo[2,3-e]isoindole-3,4′-piperidin]-7-yl)piperidine-2,6-dione (45.13 mg, 56.49 μmol, 43.3%) as a white solid. LC-MS purity: 98.8% (UV at 254 nm), 799.5 [M+H]⁺. ¹H NMR (400 MHZ, DMSO) δ 10.97 (s, 1H), 8.20 (s, 1H), 7.40 (d, J=7.6 Hz, 1H), 7.26 (d, J=7.6 Hz, 1H), 7.13-7.05 (m, 3H), 6.76 (d, J=6.2 Hz, 2H), 6.62-6.54 (m, 2H), 6.49-6.44 (m, 1H), 6.23-6.15 (m, 2H), 5.12-4.98 (m, 1H), 4.67 (d, J=5.4 Hz, 1H), 4.51 (t, J=10.0 Hz, 2H), 4.40-4.33 (m, 1H), 4.26-4.16 (m, 1H), 3.00-2.81 (m, 9H), 2.68-2.54 (m, 3H), 2.47-2.31 (m, 2H), 2.24-2.14 (m, 3H), 2.05-1.88 (m, 5H), 1.82-1.74 (m, 2H), 1.70-1,54 (m, 4H), 1.31-1,17 (m, 2H),

### Compound A94: (R)-N-((S)-2,6-dioxopiperidin-3-yl)-3-((1-(2-fluoro-4-((1S,2S)-6-hydroxy-2-phenyl-1,2,3,4-tetrahydronaphthalen-1-yl)-5-methoxyphenyl)piperidin-4-yl)methyl)-1,2,3,4,4a,5-hexahydropyrazino[1,2-d]pyrido[2,3-b][1,4]oxazine-8-carboxamide

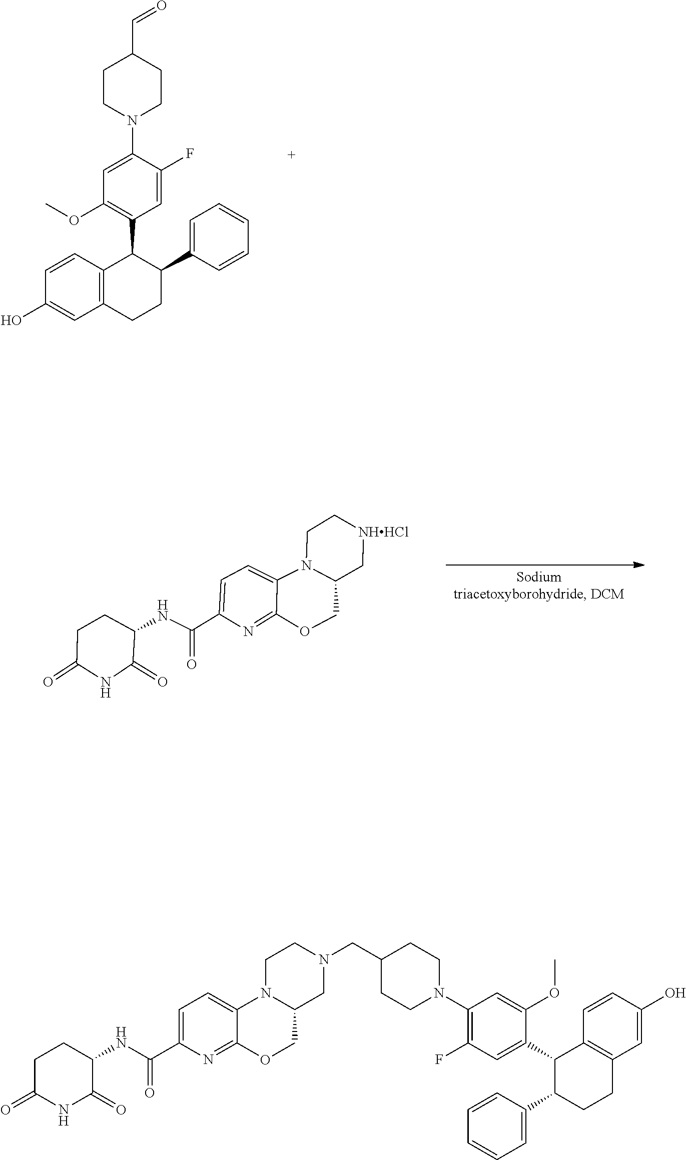

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (50.0 mg, 109 μmol, 1 eq.), (R)-N-((S)-2,6-dioxopiperidin-3-yl)-1,2,3,4,4a,5-hexahydropyrazino[1,2-d]pyrido[2,3-b][1,4]oxazine-8-carboxamide hydrochloride (59.8 mg., 153 μmol, 1.5 eq.) TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (55 mg, 0.26 mmol, 2 eq.). The mixture was stirred at room temperature for 30 minutes and concentrated. The residue was by Prep-HPLC(acetonitrile/0.05% formic acid) to afford (R)-N-((S)-2,6-dioxopiperidin-3-yl)-3-((1-(2-fluoro-4-((1S,2S)-6-hydroxy-2-phenyl-1,2,3,4-tetrahydronaphthalen-1-yl)-5-methoxyphenyl)piperidin-4-yl)methyl)-1,2,3,4,4a,5-hexahydropyrazino[1,2-d]pyrido[2,3-b][1,4]oxazine-8-carboxamide (63.87 mg, 80.96 μmol, 62.0%) as a white solid. LC-MS purity: 95.5% (UV at 254 nm), 789.5 [M+H]⁺, ¹H NMR (400 MHZ. DMSO) δ 10.84 (s, 1H), 8.50 (d, J=8.2 Hz, 1H), 7.57 (d, J=8.2 Hz, 1H), 7.31 (d, J=8.4 Hz, 1H), 7.09 (d, J=6.6 Hz, 3H), 6.76 (d, J=6.2 Hz, 2H), 6.61-6.53 (m, 2H), 6.49 (d, J=8.2 Hz, 1H), 6.25-6.13 (m, 2H), 4.81-4.63 (m, 2H), 4.44 (d, J=8.4 Hz, 1H), 4.13-3.98 (m, 1H), 3.84-3.76 (m, 1H), 3.26-3,20 (m, 4H), 2.99-2.93 (m, 6H), 2.81-2,72 (m, 2H), 2.65-2.54 (m, 2H), 2.30-2.07 (m, 6H), 2.02-1.95 (m, 1H), 1.83-1,59 (m, 6H), 1.32-1,18 (m, 3H),

### Compound A97: (S)-3-((R)-7-((1-(5-fluoro-4-((1R,2R)-6-hydroxy-2-phenyl-1,2,3,4-tetrahydronaphthalen-1-yl)-2-methoxyphenyl)piperidin-4-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

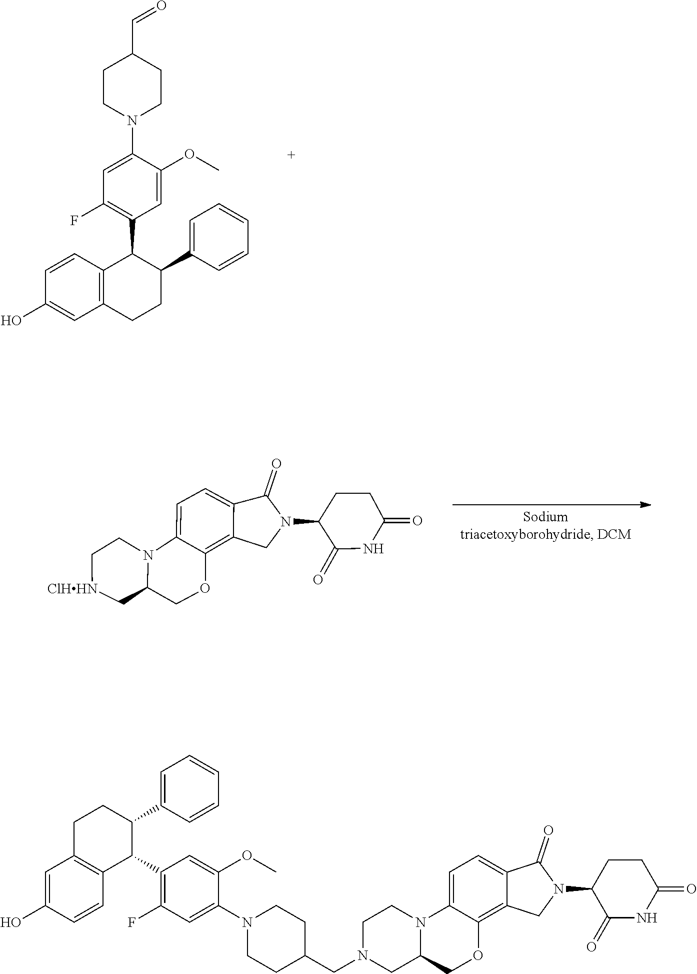

To a mixture of 1-(5-fluoro-4-((1R,2R)-6-hydroxy-2-phenyl-1,2,3,4-tetrahydronaphthalen-1-yl)-2-methoxyphenyl)piperidine-4-carbaldehyde (30.0 mg, 65.3 μmol, 1 eq.) (S)-3-((R)-1-oxo-1,3,5,5a,6,7,8,9-octahydro-2H-pyrazino[1′0.2′: 4,5][1,4]oxazino[2,3-e]isoindol-2-yl)piperidine-2,6-dione hydrochloride (49.8 mg,, 163 μmol, 1.5 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (46 mg, 0.22 mmol, 2 eq.). The mixture was stirred at room temperature for 30 minutes and concentrated. The residue was by Prep-HPLC(acetonitrile/0.05% formic acid) to afford (S)-3-((R)-7-((1-(5-fluoro-4-((1R,2R)-6-hydroxy-2-phenyl-1,2,3,4-tetrahydronaphthalen-1-yl)-2-methoxyphenyl)piperidin-4-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (24.57 mg, 30.71 μmol, 47.1%) as a white solid. LC-MS purity: 98.2% (UV at 254 nm), 800.5 [M+H]⁺. ¹H NMR (400 MHZ, DMSO) δ 10.93 (s, 1H), 7.17 (d, J=8.2 Hz, 1H), 7.14-7.07 (m, 3H), 7.01 (d, J=8.6 Hz, 1H), 6.87-6.81 (m, 2H), 6.69-6.58 (m, 2H), 6.55-6.47 (m, 1H), 6.26 (d, J=12.0 Hz, 1H), 6.06 (d, J=7.0 Hz, 1H), 5.07-4.98 (m, 1H), 4.45 (d, J=5.2 Hz, 1H), 4.40-4.33 (m, 1H), 4.30-4,22 (m, 1H), 4.14-4.07 (m, 1H), 4.02-3.94 (m, 1H), 3.86-3.78 (m, 1H), 3.47 (s, 3H), 3.28-3.16 (m, 4H), 3.04-2.88 (m, 5H), 2.77-2.65 (m, 1H), 2.62-2.55 (m, 1H), 2.46-2.28 (m, 4H), 2.27-2.16 (m, 3H), 2.14-2.05 (m, 1H), 1.98-1.92 (m, 1H), 1.80-1.69 (m, 4H), 1.65-1,56 (m, 1H), 1.29-1,14 (m, 2H) . . .

### Compound A98: (S)-3-((R)-7-((1-(5-fluoro-4-((1S,2S)-6-hydroxy-2-phenyl-1,2,3,4-tetrahydronaphthalen-1-yl)-2-methoxyphenyl)piperidin-4-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

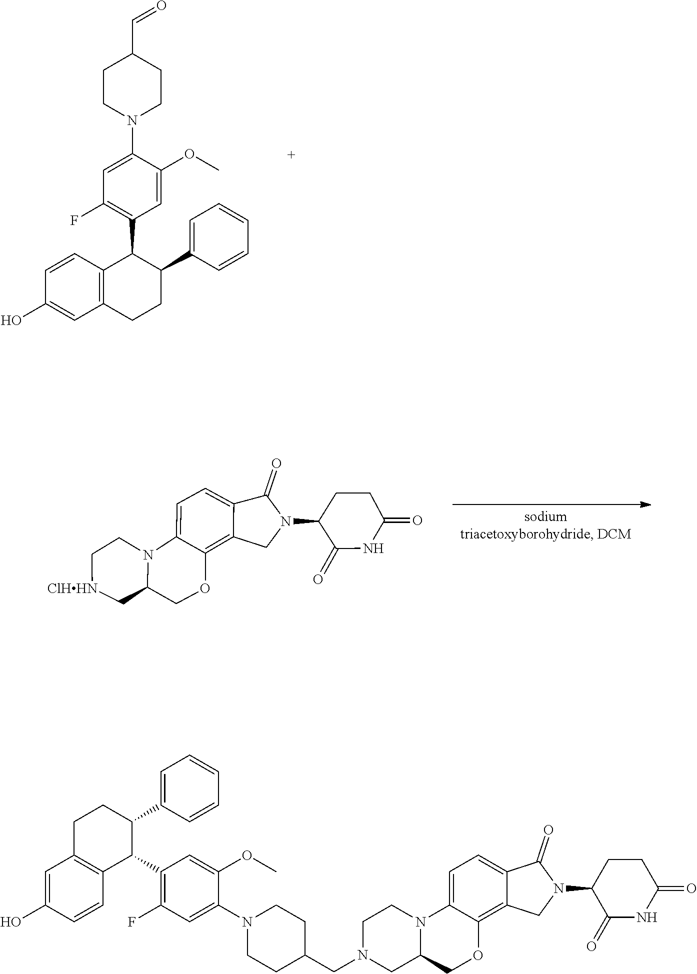

To a mixture of 1-(5-fluoro-4-((1S,2S)-6-hydroxy-2-phenyl-1,2,3,4-tetrahydronaphthalen-1-yl)-2-methoxyphenyl)piperidine-4-carbaldehyde (30.0 mg, 65.3 μmol, 1 eq.), (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione hydrochloride (30.8 mg, 78.3 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (28 mg, 0.13 mmol, 2 eq.). The mixture was stirred at room temperature for 30 minutes and concentrated. The residue was by Prep-HPLC(acetonitrile/0.05% formic acid) afford (S)-3-((R)-7-((1-(5-fluoro-4-((1S,2S)-6-hydroxy-2-phenyl-1,2,3,4-tetrahydronaphthalen-1-yl)-2-methoxyphenyl)piperidin-4-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (24.03 mg, 30.04 μmol, 46.0%) as a white solid. LC-MS purity: 97.8% (UV at 254 nm), 800.5 [M+H]⁺. ¹H NMR (400 MHZ, DMSO) δ 10.93 (s, 1H), 7.17 (d, J=8.2 Hz, 1H), 7.13-7.07 (m, 3H), 7.04-6.98 (m, 1H), 6.91-6.82 (m, 2H), 6.67-6.58 (m, 2H), 6.53-6.46 (m, 1H), 6.29-6.22 (m, 1H), 6.06 (d, J=7.0 Hz, 1H), 5.08-4.98 (m, 1H), 4.45 (d, J=5.2 Hz, 1H), 4.40-4.33 (m, 1H), 4.29-4,21 (m, 1H), 4.16-4.05 (m, 1H), 4.00-3.94 (m, 1H), 3.86-3.76 (m, 1H), 3.47 (s, 3H), 3.36-3,28 (m, 4H), 3.04-2.81 (m, 6H), 2.77-2.66 (m, 1H), 2.61-2.54 (m, 1H), 2.46-2.33 (m, 3H), 2.28-2.16 (m, 3H), 2.13-2.06 (m, 1H), 1.98-1.91 (m, 1H), 1.80-1.69 (m, 4H), 1.64-1,52 (m, 1H), 1.28-1,13 (m, 2H).

### Compound A99: (S)-3-((R)-7-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

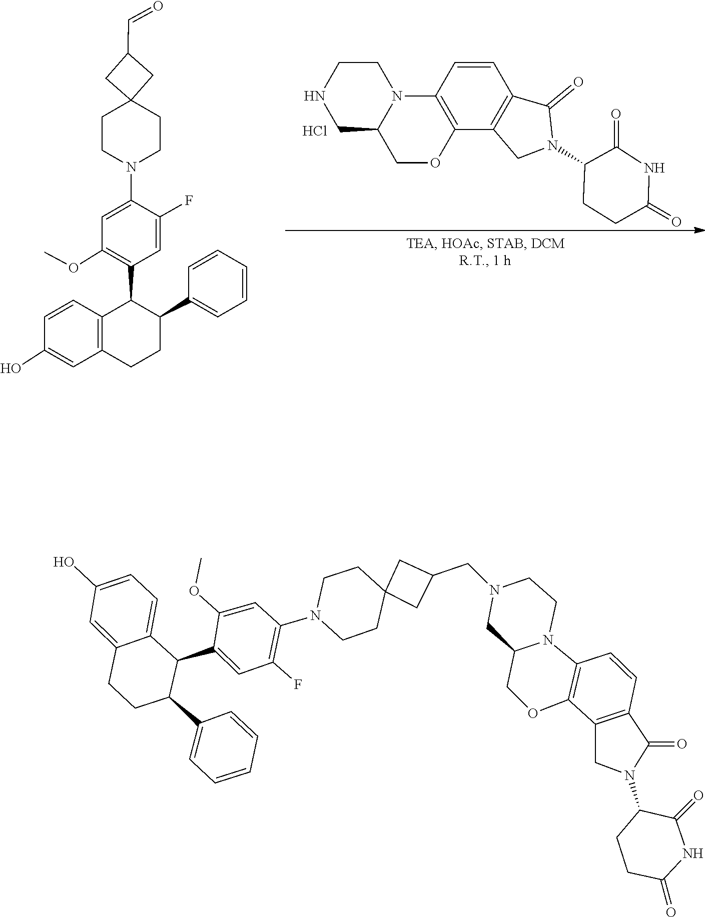 [SMILES: COc1cc(N2CCC3(CC2)CC(C=O)C3)c(F)cc1[C@@H]1c2ccc(O)cc2CC[C@@H]1c1ccccc1 ; Cl ; O=C1CC[C@H](N2Cc3c(ccc4c3OC[C@H]3CNCCN43)C2=O)C(=O)N1 ; COc1cc(N2CCC3(CC2)CC(CN2CCN4c5ccc6c(c5OC[C@H]4C2)CN([C@H]2CCC(=O)NC2=O)C6=O)C3)c(F)cc1[C@@H]1c2ccc(O)cc2CC[C@@H]1c1ccccc1]

To a mixture of 7-(2-fluoro-4-((1S,2S)-6-hydroxy-2-phenyl-1,2,3,4-tetrahydronaphthalen-1-yl)-5-methoxyphenyl)-7-azaspiro[3.5]nonane-2-carbaldehyde (60 mg, 0.12 mmol, 1 eq.), (S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione hydrochloride (57 mg, 0.144 mmol, 1,2 eq.), TEA (18 mg, 0.18 mmol, 1.5 eq.) in DCM (5.0 mL) was added acetic acid (12 mg, 0.2 mmol, 1.7 eq.) followed by sodium triacetoxyborohydride (51 mg, 0.24 mmol, 2 eq.). The mixture was stirred at room temperature for 30 minutes and concentrated. The residue was purified by reverse-phase chromatography (acetonitrile/0.05% formic acid) to afford (S)-3-((R)-7-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-1-oxo-1,3,5,5a,6,7,8,9-octahydro-2H-pyrazino[1′0.2′: 4,5][1,4]oxazino[2,3-e]isoindol-2-yl)piperidine-2,6-dione (54.58 mg, 54.1% yield) as white solid. LC-MS purity: 100% (UV at 254 nm), 840.9 [M+H]⁺

¹H NMR (400 MHz, DMSO) δ 10.93 (s, 1H), 7.21-6.98 (m, 5H), 6.75 (d, J=6.4 Hz, 2H), 6.63-6.45 (m, 3H), 6.18 (dd, J=10.4, 6.8 Hz, 2H), 5.03 (dd, J=13.2, 4.8 Hz, 1H), 4.67 (d, J=4.8 Hz, 1H), 4.30 (dd, J=34,4, 13.6 Hz, 2H), 4.10 (d, J=16.8 Hz, 1H), 3.95 (dd, J=8.4, 6.8 Hz, 1H), 3.80 (d, J=11,2 Hz, 1H), 3.25-3.03 (m, 4H), 2.95 (s, 3H), 2.92-2.69 (m, 9H), 2.57 (d. J=16.8 Hz, 1H), 2.43-1.92 (m, 8H), 1.77-1,40 (m, 8H).

### Compound A100: (S)-3-(1′-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione

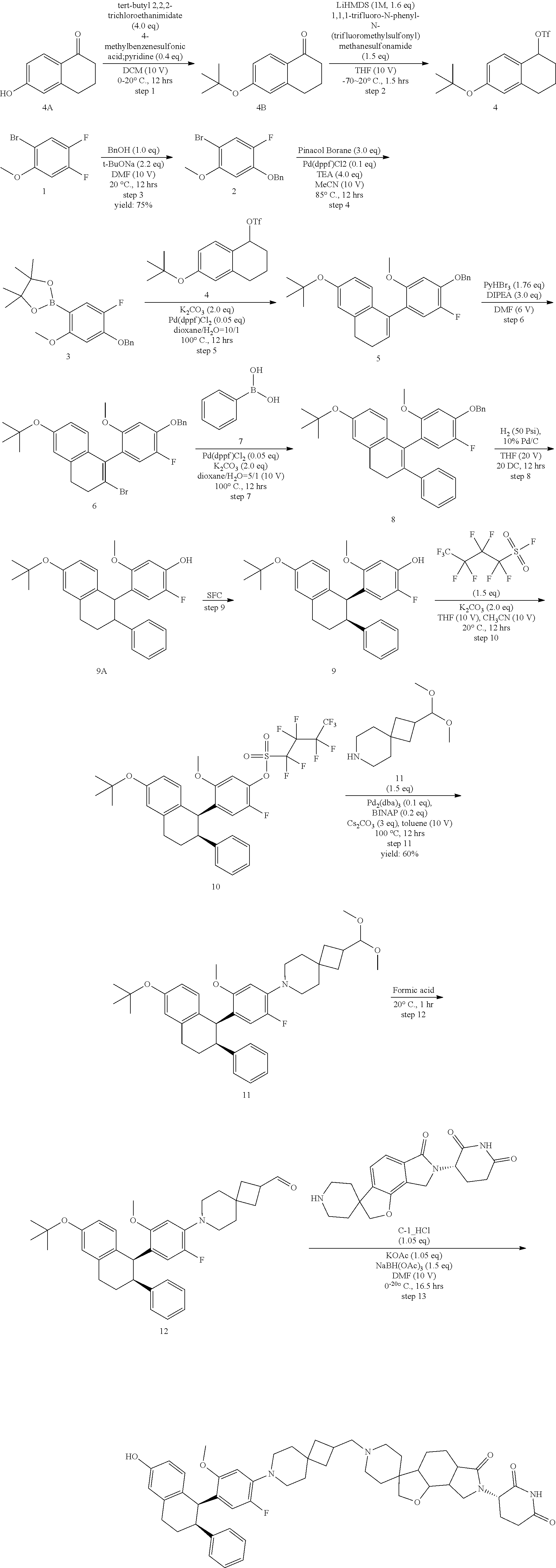

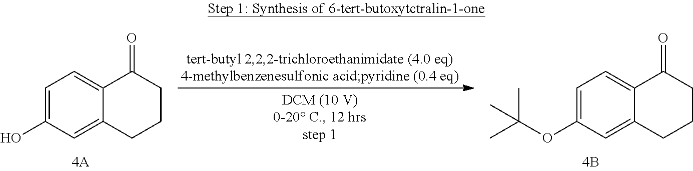 [SMILES: O=C1CCCc2cc(O)ccc21 ; CC(C)(C)Oc1ccc2c(c1)CCCC2=O]

To the solution of compound 4A (200 g, 1,23 mol, 1.00 eq) in DCM (2 L) was added 4-methylbenzenesulfonic acid; pyridine (124 g, 493 mmol, 0.40 eq) at 0° C., before tert-butyl 2,2,2-trichloroethanimidate (1.08 kg, 4.93 mol, 883 mL, 4.00 eq) was added dropwise at 0° C. under N₂ atmosphere. During which the temperature was maintained below 5° C. The reaction mixture was warmed to 20° C. and stirred at 20° C. for 12 hrs. The mixture was filtered, and the filter cake was washed with DCM (500 mL*2). The residue was added H₂O (500 mL) and stirred for 15 min, before the aqueous phase was extracted with DCM (500 mL*2). The combined organic phase was washed with brine (300 mL), dried over anhydrous Na₂SO₄, filtered, and concentrated in vacuum. The residue was purified by column chromatography (SiO₂, Petroleum ether/EtOAc=100/1 to 1/1). Compound 4B (297 g, 1,27 mol, 51.3% yield, 93.0% purity) was obtained as a red oil, which was indicated by HNMR.

¹H NMR (400 MHZ, CDCl₃) δ 7.88 (d, J=8.6 Hz, 1H), 6.82 (dd, J=2.0, 8.6 Hz, 1H), 6.73 (s, 1H), 2.83 (t, J=6.1 Hz, 2H), 2.53 (t, J=6.6 Hz, 2H), 2.04 (quin, J=6.3 Hz, 2H), 1.34 (s, 9H), Step 2: Synthesis of (6-tert-butoxy-3,4-dihydronaphthalen-1-yl)trifluoromethanesulfonate

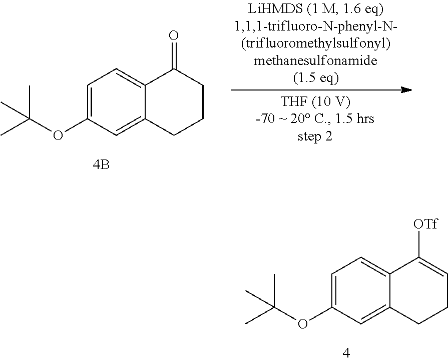 [SMILES: CC(C)(C)Oc1ccc2c(c1)CCCC2=O ; CC(C)(C)Oc1ccc2c(c1)CCC=C2OS(=O)(=O)C(F)(F)F]

To the solution of compound 4B (100 g, 458 mmol, 1.00 eq) in THF (1 L) was added LiHMDS (1 M, 733 mL, 1.60 eq) dropwise at −70° C. under N₂ atmosphere. The reaction mixture was stirred at −70° C. for 30 min, before 1,1,1-trifluoro-N-phenyl-N-(trifluoromethylsulfonyl)methanesulfonamide (245 g, 687 mmol, 1.50 eq) was added at −70° C. The reaction mixture was slowly warmed to 20° C. and stirred for 1 hour. The mixture was added into H₂O (500 mL) and stirred for further 15 min. The aqueous phase was extracted with EtOAc (500 mL*2). The combined organic layer was washed with brine (500 mL), dried with anhydrous Na₂SO₄, filtered and concentrated in vacuum. The residue was purified by silica gel column chromatography (column height: 250 mm, diameter: 100 mm, 100-200 mesh silica gel, Petroleum ether/EtOAc=100/1 to 1/1). Compound 4 (136 g, 365 mmol, 79.6% yield, 94.0% purity) was obtained as a yellow solid, which was indicated by HNMR.

¹H NMR (400 MHZ, CDCl₃) δ 7.19-7.14 (m, 1H), 6.79 (dd, J=2,3, 8.4 Hz, 1H), 6.74-6.70 (m, 1H), 5.83 (t, J=4.8 Hz, 1H), 2.74 (t, J=8.2 Hz, 2H), 2.41 (dt, J=4.8, 8.1 Hz, 2H), 1.29 (s, 9H),

### Step 1: Synthesis of 1-benzyloxy-4-bromo-2-fluoro-5-methoxy-benzene

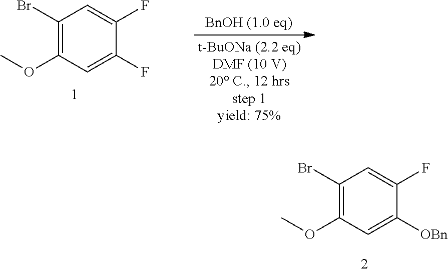 [SMILES: COc1cc(F)c(F)cc1Br ; COc1cc(OCc2ccccc2)c(F)cc1Br]

To the solution of compound 1 (74.0 g, 332 mmol, 1.00 eq) in DMF (740 mL) was added BnOH (43.1 g, 398 mmol, 41,4 mL, 1,20 eq) dropwise at 10˜20° C. t-BuONa (70.2 g, 730 mmol, 2.20 eq) was added to the mixture in portions at 10˜20° C., and the mixture was stirred at 20° C. for 12 hrs and turned to a green solution. The reaction mixture was concentrated under reduced pressure to give a residue. The residue was dissolved in MTBE (1 L) and H₂O (500 mL) and extracted with MTBE (300 mL*3). The combined organic layer was washed with brine (500 mL*3), dried over anhydrous Na₂SO₄, filtered and concentrated under reduced pressure to give a residue. The residue was purified by column chromatography (SiO₂, Petroleum ether/EtOAc=50/1 to 5/1). Compound 2 (100 g, 321 mmol, 96.8% yield) was obtained as an off-white solid, which was indicated by HNMR.

¹H NMR (400 MHZ, CDCl₃) δ 7.14-7.44 (m, 6H), 6.50 (d, J=7.25 Hz, 1H), 5.06 (s, 2H), 3.70 (s, 3H),

### Step 2: Synthesis of 2-(4-benzyloxy-5-fluoro-2-methoxy-phenyl)-4,4,5,5-tetramethyl-1,3,2-dioxaborolane

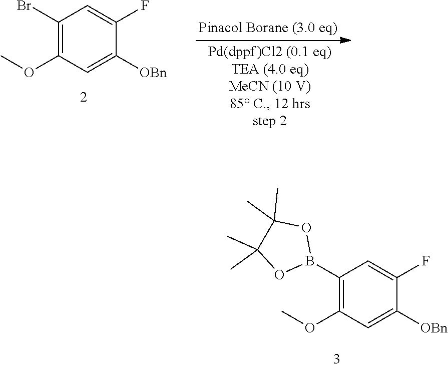 [SMILES: COc1cc(OCc2ccccc2)c(F)cc1Br ; COc1cc(OCc2ccccc2)c(F)cc1B1OC(C)(C)C(C)(C)O1]

The mixture of compound 2 (110 g, 353 mmol, 1.00 eq), 4,4,5,5-tetramethyl-1,3,2-dioxaborolane (136 g, 1.06 mol, 154 mL, 3.00 eq), Pd(dppf)Cl₂ (25.8 g, 35.4 mmol, 0.10 eq) and TEA (143 g, 1,41 mol, 197 mL, 4.00 eq) in MeCN (2.2 L) was degassed and purged with N₂ for 3 times at 20° C., before the reaction mixture was stirred at 85° C. for 5 hrs under N₂ atmosphere. The reaction mixture was filtered and concentrated under reduced pressure to give a residue. The residue was diluted with EtOAc (500 mL) and extracted with water (500 mL*2). The combined organic layer was washed with brine (400 mL), dried over anhydrous Na₂SO₄, filtered and concentrated under reduced pressure to give a residue. The residue was purified by column chromatography (SiO₂, Petroleum ether/EtOAc=100/1 to 5/1). The residue was stirred in petroleum ether (200 mL) at 20° C. for 1 hr and precipitation was observed. The resulting suspension was filtered and concentrated under reduced pressure to give the product. Compound 3 (151 g, 379 mmol, 53.7% yield, 90.0% purity) was obtained as a white solid, which was indicated by HNMR.

¹H NMR (400 MHZ, CDCl₃) δ 7.38-7.20 (m, 6H), 6.42 (d, J=6.5 Hz, 1H), 5.09 (s, 2H), 3.66 (s, 3H), 1.25 (s, 11H), m/e+1=359.

### Step 3: Synthesis of 4-(4-benzyloxy-5-fluoro-2-methoxy-phenyl)-7-tert-butoxy-1,2-dihydronaphthalene

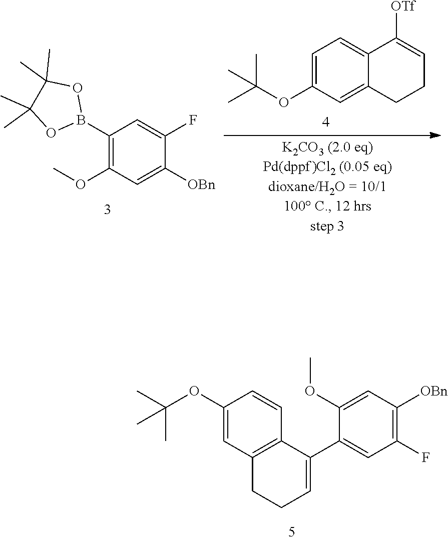

The mixture of compound 4 (112 g, 321 mmol, 1 eq), compound 3 (115 g, 321 mmol, 1 eq), Pd(dppf)Cl₂ (11.8 g, 16.0 mmol, 0.05 eq) and K₂CO₃ (68.1 g, 642 mmol, 2.00 eq) in dioxane (1120 mL) and H₂O (224 mL) was degassed and purged with N₂ for 3 times at 20° C., before the mixture was stirred at 100° C. for 12 hrs under N₂ atmosphere. The reaction mixture was filtered and concentrated under reduced pressure to give a residue. The residue was purified by column chromatography (SiO₂, Petroleum ether/EtOAc=100/1 to 10/1). Compound 5 (57.0 g, 98.3 mmol, 30.6% yield, 74.6% purity) was obtained as a yellow oil, m/e+1=433.

### Step 4: Synthesis of 4-(4-benzyloxy-5-fluoro-2-methoxy-phenyl)-3-bromo-7-tert-butoxy-1,2-dihydronaphthalene

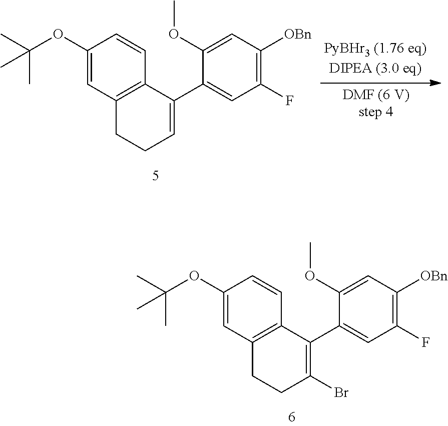 [SMILES: COc1cc(OCc2ccccc2)c(F)cc1C1=CCCc2cc(OC(C)(C)C)ccc21 ; COc1cc(OCc2ccccc2)c(F)cc1C1=C(Br)CCc2cc(OC(C)(C)C)ccc21]

To the solution of compound 5 (81.0 g, 187 mmol, 1.00 eq) in DMF (486 mL) was added DIEA (72.6 g, 562 mmol, 97.9 mL, 3.00 eq) at 20° C. PyBr₃ (105 g, 329 mmol, 1.76 eq) was added dropwise to the solution at 5˜10° C. under N₂ atmosphere. The mixture was stirred at 5˜10° C. for 18 hrs under N₂ and turned to a yellow solution. The reaction mixture was concentrated under reduced pressure to give a residue. The residue was dissolved in EtOAc (400 mL) and H₂O (200 mL), and the resulting mixture was extracted with EtOAc (200 mL*5). The combined organic layer was washed with brine (400 mL*3), dried over anhydrous Na₂SO₄, filtered and concentrated under reduced pressure to give a residue. The residue was purified by column chromatography (SiO₂, Petroleum ether/EtOAc=100/1 to 10/1). Compound 6 (72.0 g, 114.04 mmol, 60.9% yield, 81.0% purity) was obtained as a yellow oil, which was detected by HNMR.

¹H NMR (400 MHZ, CDCl₃) δ 7.50 (br d, J=7.03 Hz, 2H), 7.33-7.44 (m, 3H), 6.88 (br d, J=11.32 Hz, 1H), 6.76 (br d, J=1.79 Hz, 1H), 6.60-6.70 (m, 2H), 6.50 (br d, J=8.46 Hz, 1H), 5.12-5.28 (m, 2H), 3.68 (s, 3H), 2.84-3.09 (m, 4H), 1.34 (s, 9H), m/e+1=511. Step 5: Synthesis of 4-(4-benzyloxy-5-fluoro-2-methoxy-phenyl)-7-tert-butoxy-3-phenyl-1,2-dihydronaphthalene

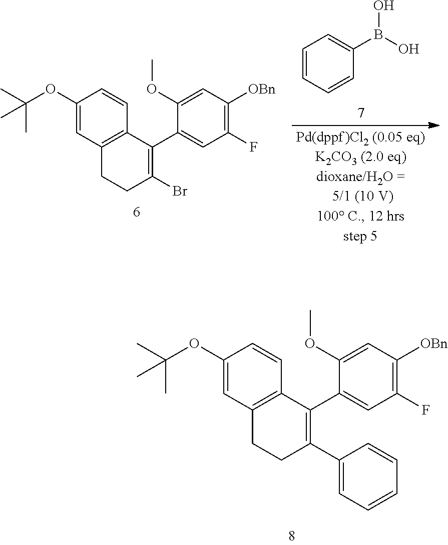 [SMILES: COc1cc(OCc2ccccc2)c(F)cc1C1=C(Br)CCc2cc(OC(C)(C)C)ccc21 ; OB(O)c1ccccc1 ; COc1cc(OCc2ccccc2)c(F)cc1C1=C(c2ccccc2)CCc2cc(OC(C)(C)C)ccc21]

The mixture of compound 6 (72.0 g, 141 mmol, 1.00 eq), phenylboronic acid (18.9 g, 155 mmol, 1,10 eq), Pd(dppf)Cl₂ (5.15 g, 7.04 mmol, 0.05 eq) and K₂CO₃ (38.9 g, 282 mmol, 2.00 eq) in dioxane (720 mL) was degassed and purged with N₂ for 3 times at 20° C., before the mixture was stirred at 100° C. for 12 hrs under N₂ atmosphere. The reaction mixture was filtered and concentrated under reduced pressure to give a residue. The residue was purified by column chromatography (SiO₂, Petroleum ether/EtOAc=100/1 to 10/1). The residue was stirred in MeOH (350 mL) at 20° C. for 3 hrs and then filtered to give the product. Compound 8 (80.0 g, crude) was obtained as a light yellow solid, which was detected by HNMR.

¹H NMR (400 MHZ, CDCl₃) δ 7.22-7.38 (m, 5H), 6.91-7.07 (m, 5H), 6.74 (d, J=2.25 Hz, 1H), 6.56-6.69 (m, 2H), 6.48-6.56 (m, 1H), 6.38 (d, J=7.13 Hz, 1H), 5.04 (s, 2H), 3.33 (s, 3H), 2.81-2.90 (m, 2H), 2.63-2,78 (m, 2H), 1.28 (s, 9H), m/e+1=509.

### Step 6: Synthesis of 4-(6-tert-butoxy-2-phenyl-tetralin-1-yl)-2-fluoro-5-methoxy-phenol

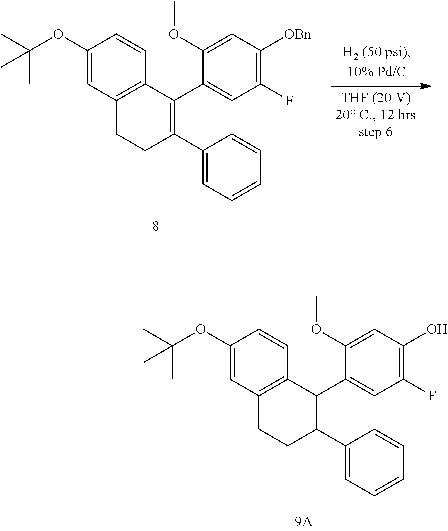 [SMILES: COc1cc(OCc2ccccc2)c(F)cc1C1=C(c2ccccc2)CCc2cc(OC(C)(C)C)ccc21 ; COc1cc(O)c(F)cc1C1c2ccc(OC(C)(C)C)cc2CCC1c1ccccc1]

The mixture of compound 8 (20.0 g, 39.3 mmol, 1.00 eq) and Pd/C(15.0 g, 39.3 mmol, 10% purity, 1.00 eq) in THF (420 mL) was degassed and purged with H₂ for 3 times, before the mixture was stirred at 20° C. for 12 hrs under H₂ atmosphere (50 Psi). The reaction mixture was filtered and concentrated under reduced pressure to give a residue. The residue was stirred in petroleum ether (200 mL) at 20° C. for 3 hrs and the mixture was filtered to give the product. Compound 9A (15.0 g, 31.7 mmol, 80.6% yield, 88.8% purity) was obtained as a white solid.

### Step 7: Synthesis of 4-[(1S,2S)-6-tert-butoxy-2-phenyl-tetralin-1-yl]-2-fluoro-5-methoxy-phenol

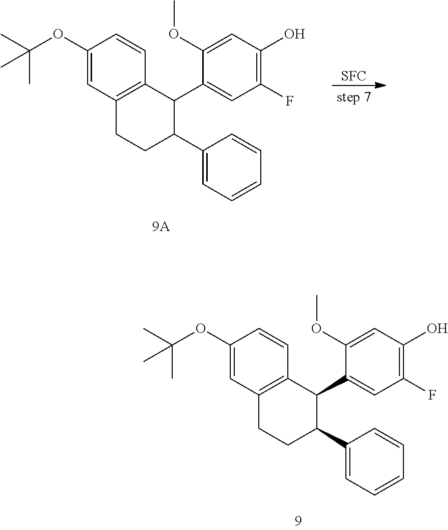 [SMILES: COc1cc(O)c(F)cc1C1c2ccc(OC(C)(C)C)cc2CCC1c1ccccc1 ; "SFC" ; COc1cc(O)c(F)cc1[C@@H]1c2ccc(OC(C)(C)C)cc2CC[C@@H]1c1ccccc1]

Compound 9A (10.0 g) was purified by chiral SFC(column: DAICEL CHIRALPAK AD (250 mm*50 mm, 10 um); mobile phase: [0.1% NH₃H₂O IPA]; B %: 50%-50%, 6 min).

Compound 9 (4.00 g, 7.1 mmol, 76.7% yield) was obtained as a yellow solid, which was indicated by HNMR.

¹H NMR (400 MHZ, CDCl₃) δ6.95-7.09 (m, 3H), 6.60-6.81 (m, 5H), 6.26 (d, J=11.76 Hz, 1H), 6.08 (d, J=7.50 Hz, 1H), 4.94 (d, J=3.88 Hz, 1H), 4.75 (d, J=5.25 Hz, 1H), 3.24 (ddd, J=13.20, 5.32, 2.13 Hz, 1H), 2.84-3.03 (m, 5H), 2.06-2.25 (m, 1H), 1.64-1.74 (m, 1H), 1.29 (s, 9H),

### Step 8: Synthesis of [4-[(1S,2S)-6-tert-butoxy-2-phenyl-tetralin-1-yl]-2-fluoro-5-methoxy-phenyl] 1,1,2,2,3,3,4,4,4-nonafluorobutane-1-sulfonate

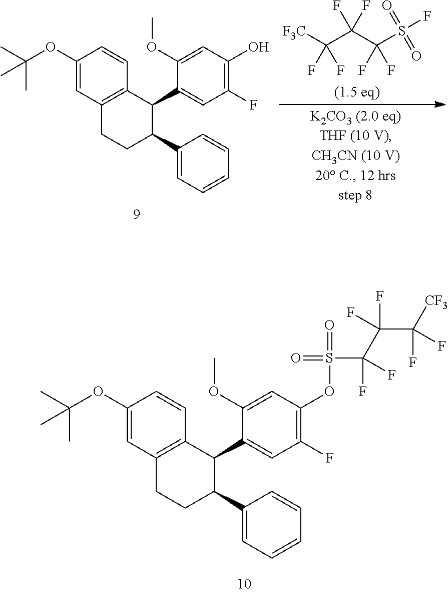 [SMILES: COc1cc(O)c(F)cc1[C@@H]1c2ccc(OC(C)(C)C)cc2CC[C@@H]1c1ccccc1 ; O=S(=O)(F)C(F)(F)C(F)(F)C(F)(F)C(F)(F)F ; COc1cc(OS(=O)(=O)C(F)(F)C(F)(F)C(F)(F)C(F)(F)F)c(F)cc1[C@@H]1c2ccc(OC(C)(C)C)cc2CC[C@@H]1c1ccccc1]

To the solution of compound 9 (4.50 g, 10.7 mmol, 1.00 eq) in MeCN (45 mL) and THF (45 mL) was added K₂CO₃ (2.22 g, 16.05 mmol, 1.50 eq) and 1,1,2,2,3,3,4,4,4-nonafluorobutane-1-sulfonyl fluoride (4.85 g, 16.1 mmol, 2.82 mL, 1.50 eq) at 20° C. The mixture was stirred at 20° C. for 12 hrs and turned to a light yellow suspension. The reaction mixture was filtered and concentrated under reduced pressure to give the crude product. Compound 10 (8.00 g, crude) was obtained as a light yellow solid.

### Step 9: Synthesis of 7-[4-[(1S,2S)-6-tert-butoxy-2-phenyl-tetralin-1-yl]-2-fluoro-5-methoxy-phenyl]-2-(dimethoxymethyl)-7-azaspiro[3.5]nonane

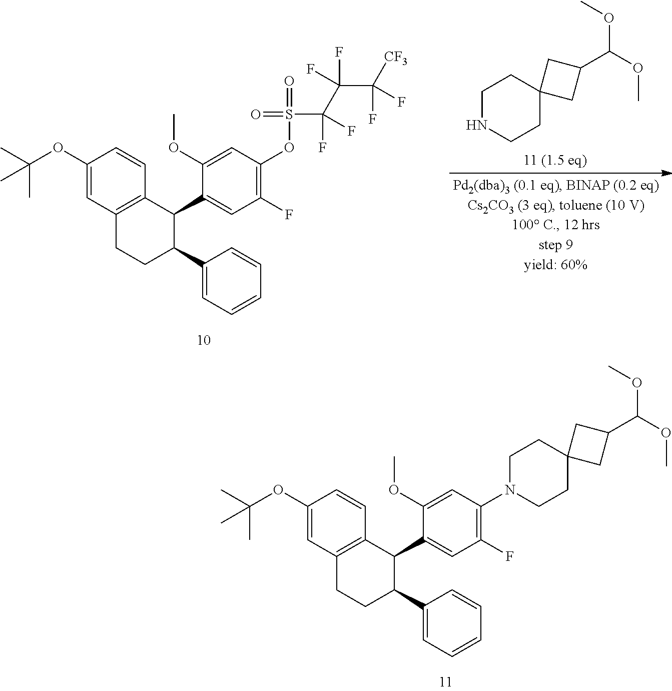 [SMILES: COc1cc(OS(=O)(=O)C(F)(F)C(F)(F)C(F)(F)C(F)(F)F)c(F)cc1[C@@H]1c2ccc(OC(C)(C)C)cc2CC[C@@H]1c1ccccc1 ; COC(OC)C1CC2(CCNCC2)C1 ; COc1cc(N2CCC3(CC2)CC(C(OC)OC)C3)c(F)cc1[C@@H]1c2ccc(OC(C)(C)C)cc2CC[C@@H]1c1ccccc1]

The mixture of compound 10 (5.20 g, 7.40 mmol, 1.00 eq), compound 11 (2.21 g, 11,1 mmol, 1.50 eq), Pd₂(dba) 3 (678 mg, 740 μmol, 0.10 eq), Cs₂CO₃ (7.23 g, 22.2 mmol, 3.00 eq) and BINAP (921 mg, 1,48 mmol, 0.20 eq) in toluene (52 mL) was degassed and purged with N₂ for 3 times at 20° C., before the mixture was stirred at 100° C. for 12 hrs under N₂ atmosphere. The reaction mixture was filtered and concentrated under reduced pressure to give a residue. The residue was purified by column chromatography (SiO₂, Petroleum ether/EtOAc=50/1 to 5/1). Compound 11 (3.00 g, 4.99 mmol, 67.3% yield) was obtained as a white solid.

### Step 10: Synthesis of 7-[2-fluoro-4-[(1S,2S)-6-hydroxy-2-phenyl-tetralin-1-yl]-5-methoxy-phenyl]-7-azaspiro[3.5]nonane-2-carbaldehyde

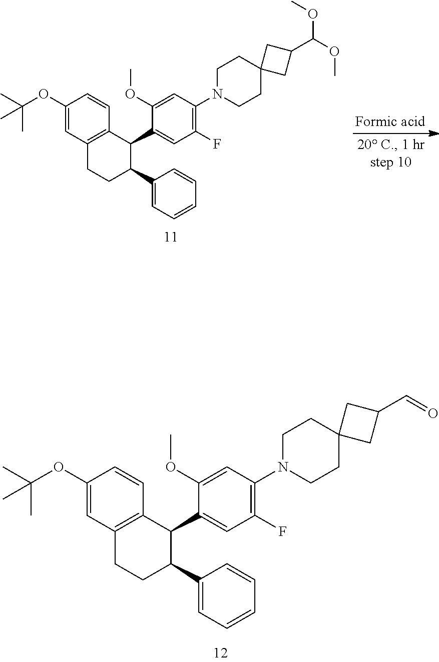

Compound 11 (500 mg, 831 μmol, 1.00 eq) was stirred in formic acid (5 mL) at 20° C. for 1 hr, and the mixture turned to a yellow solution. The mixture was concentrated under reduced pressure to give the product (˜ 2 mL solution). Compound 12 was obtained as a yellow solution. Step 11: Synthesis of (3S)-3-[1′-[[7-[2-fluoro-4-[(1S,2S)-6-hydroxy-2-phenyl-tetralin-1-yl]-5-methoxy-phenyl]-7-azaspiro[3.5]nonan-2-yl]methyl]-6-oxo-spiro[2,8-dihydrofuro[2,3-e]isoindole-3,4′-piperidine]-7-yl]piperidine-2,6-dione

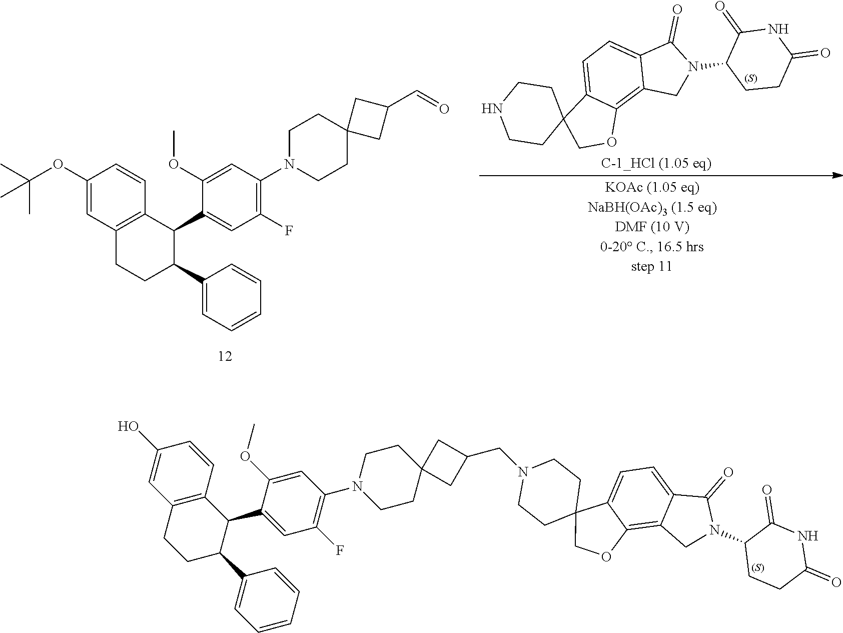

To the suspension of C-1_HCl (342 mg, 872 μmol, 1.05 eq, HCl) in DMF (5 mL) was added KOAc (85,6 mg, 872.17 μmol, 1.05 eq) in one portion at 20° C., and stirred for 30 min, before being cooled to 0° C. NaBH (OAc) 3 (264 mg, 1,25 mmol, 1.50 eq) and the solution of compound 12 (415 mg, 830 μmol, 1.00 eq) in formic acid (2 mL) was added in portions at 0° C. The reaction mixture was stirred at 0° C. for 4 hrs and stirred at 20° C. for 12 hrs, the mixture turned to a brown solution. The reaction mixture was poured into ice-water (100 mL) at 0° C., before saturated NaHCO₃ solution was added to the mixture dropwise to adjust pH=6. White precipitate was observed, and the mixture was filtered to give the crude product. The aqueous layer was extracted with EtOAc (50 mL*5), and the combined organic layer was washed with brine (100 mL*2), dried over anhydrous Na₂SO₄, filtered and concentrated under reduced pressure to give the crude product at 40° C. The crude product was combined and stirred in MTBE (25 mL) for 30 minutes at 20° C., then the mixture was filtered to give (3S)-3-[1′-[7-[2-fluoro-4-[(1S,2S)-6-hydroxy-2-phenyl-tetralin-1-yl]-5-methoxy-phenyl]-7-azaspiro[3.5]nonan-2-yl]methyl]-6-oxo-spiro[2,8-dihydrofuro[2,3-e]isoindole-3,4′-piperidine]-7-yl]piperidine-2,6-dione (580 mg, 681 μmol, 82.1% yield, 98.6% purity) was obtained as a white solid. LC-MS: 840.9 [M+H]⁺.

¹H NMR (400 MHZ, DMSO-d₆) § 10.97 (s, 1H), 9.13 (br s, 1H), 7.22-7.43 (m, 2H), 7.00-7.15 (m, 3H), 6.68-6.81 (m, 2H), 6.41-6.62 (m, 3H), 6.11-6.24 (m, 2H), 5.08 (dd, J=13.32, 4.94 Hz, 1H), 4.66 (br d, J=5.00 Hz, 1H), 4.52 (s, 2H), 4.35-4.36 (m, 1H), 4.37 (d, J=17.13 Hz, 1H), 4.21 (d, J=17.13 Hz, 1H), 3.24 (br s, 1H), 2.72-2.95 (m, 12H), 2.55-2.64 (m, 2H), 2.32-2.46 (m, 2H), 2.05-2.21 (m, 3H), 1.83-2.03 (m, 6H), 1.51-1.77 (m, 7H), 1.44 (br dd, J=9.26, 5,63 Hz, 2H),

### Compound A101: (S)-3-((S)-3-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

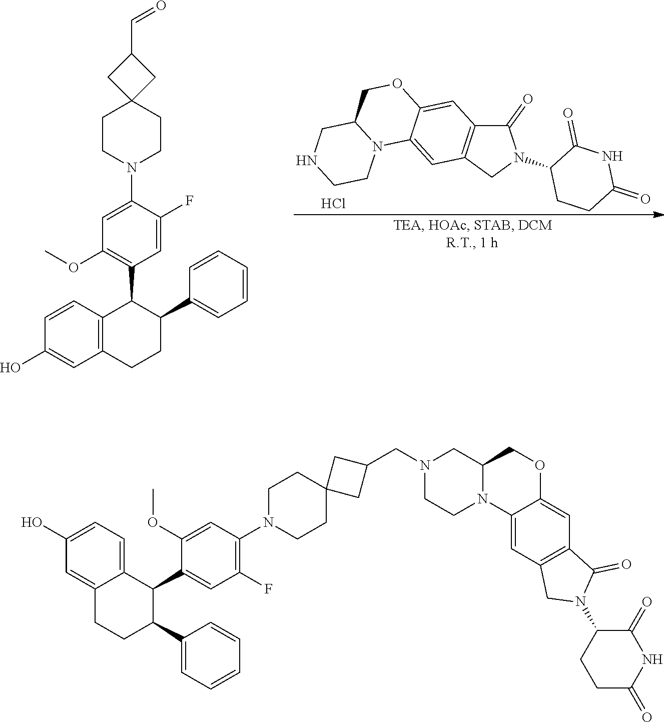 [SMILES: COc1cc(N2CCC3(CC2)CC(C=O)C3)c(F)cc1[C@@H]1c2ccc(O)cc2CC[C@@H]1c1ccccc1 ; O=C1CC[C@H](N2Cc3cc4c(cc3C2=O)OC[C@@H]2CNCCN42)C(=O)N1 ; Cl ; COc1cc(N2CCC3(CC2)CC(CN2CCN4c5cc6c(cc5OC[C@@H]4C2)C(=O)N([C@H]2CCC(=O)NC2=O)C6)C3)c(F)cc1[C@@H]1c2ccc(O)cc2CC[C@@H]1c1ccccc1]

To a mixture of 7-(2-fluoro-4-((1S,2S)-6-hydroxy-2-phenyl-1,2,3,4-tetrahydronaphthalen-1-yl)-5-methoxyphenyl)-7-azaspiro[3.5]nonane-2-carbaldehyde (60 mg, 0.12 mmol, 1 eq.), (S)-3-((S)-8-oxo-1,2,3,4,4a,5,8,10-octahydro-9H-pyrazino[11,2′: 4,5][1,4]oxazino[2,3-f]isoindol-9-yl)piperidine-2,6-dione hydrochloride (57 mg, 0.144 mmol, 1,2 eq.), TEA (18 mg, 0.18 mmol, 1.5 eq.) in DCM (5.0 mL) was added acetic acid (12 mg, 0.2 mmol, 1.7 eq.) followed by sodium triacetoxyborohydride (51 mg, 0.24 mmol, 2 eq.). The mixture was stirred at room temperature for 30 minutes and concentrated. The residue was purified by reverse-phase chromatography (acetonitrile/0.05% formic acid) to afford (S)-3-((S)-3-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (57.92 mg, 57.5% yield) as white solid. LC-MS purity: 100% (UV at 254 nm), 840.7 [M+H]⁺.

¹H NMR (400 MHZ, DMSO) δ 10.97 (s, 1H), 8.20 (s, 1H), 7.40 (d, J=7.6 Hz, 1H), 7.26 (d, J=7.6 Hz, 1H), 7.11-7.07 (m, 3H), 6.77 (d, J=7.2 Hz, 2H), 6.59-6.46 (m, 3H), 6.22-6.18 (m, 2H), 5.08 (dd, J=13.2, 5.2 Hz, 1H), 4.67 (d, J=5,6 Hz, 1H), 4.55-4.50 (m, 2H), 4.09-4.06 (m, 1H), 4.29 (dd, J=68.4, 17.2 Hz, 2H), 3.23-2.82 (m, 9H), 2.67-2.53 (m, 3H), 2.44-2.14 (m, 5H), 2.00-1.62 (m, 11H), 1.25-1,22 (m, 2H),

### Compound A102: (S)-3-((R)-7-((7-(5-fluoro-4-((1R,2R)-6-hydroxy-2-phenyl-1,2,3,4-tetrahydronaphthalen-1-yl)-2-methoxyphenyl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

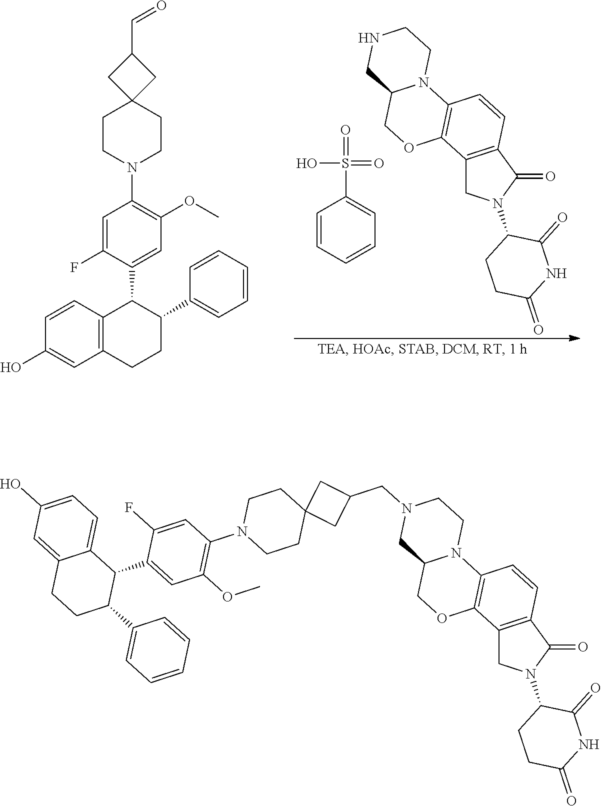

To a mixture of 7-(5-fluoro-4-((1R,2R)-6-hydroxy-2-phenyl-1,2,3,4-tetrahydronaphthalen-1-yl)-2-methoxyphenyl)-7-azaspiro[3.5]nonane-2-carbaldehyde (45.8 mg, 91.6 μmoll eq), (S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione benzenesulfonic acid (61.3 mg, 119 μmol, 1.3 eq) in DCM/DMA (2 mL) was added TEA (18.5 mg, 183 μmol, 2 eq), AcOH (16.5 mg, 275 μmol, 3 eq), sodium triacetoxyborohydride (38.8 mg, 183 μmol, 3 eq) at rt. then the mixture was stirred at rt for 1 h. The resulting mixture was concentrated under reduced pressure. The residue was purified by reverse flash chromatography with the following conditions: column, C18 silica gel; mobile phase, ACN in water, 0% to 50% gradient in 20 min; detector, UV 214 nm to afford (S)-3-((R)-7-((7-(5-fluoro-4-((1R,2R)-6-hydroxy-2-phenyl-1,2,3,4-tetrahydronaphthalen-1-yl)-2-methoxyphenyl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (25 mg, yield: 32.5%) as a white solid. LC-MS purity: 100% (UV at 254 nm), 840.6 [M+H]⁺.

¹H NMR (400 MHZ, DMSO) & 10.93 (s, 1H), 9.14 (s, 1H), 7.17 (d, J=8.4 Hz, 1H), 7.13-7.05 (m, 3H), 7.00 (d, J=8.4 Hz, 1H), 6.88-6.78 (m, 2H), 6.68-6.58 (m, 2H), 6.54-6.45 (m, 1H), 6.24 (d, J=11.6 Hz, 1H), 6.05 (d, J=7.2 Hz, 1H), 5.03 (dd, J=13.2, 5.2 Hz, 1H), 4.44 (d, J=5.2 Hz, 1H), 4.38-4.31 (m, 1H), 4.26 (d, J=16.8 Hz, 1H), 4.10 (d, J=16.8 Hz, 1H), 4.00-3.90 (m, 1H), 3.80 (d, J=11.6 Hz, 1H), 3.34-3.31 (m, 1H), 3.18-3.11 (m, 1H), 3.06-2.54 (m, 12H), 2.47-2.36 (m, 3H), 2.28-2.07 (m, 2H), 2.00-1.87 (m, 3H), 1.73 (t, J=10.4 Hz, 2H), 1.68-1.60 (m, 2H), 1.57-1,48 (m, 2H), 1.46-1.35 (m, 2H),

### Compound A103: (S)-3-((R)-7-((7-(5-fluoro-4-((1S,2S)-6-hydroxy-2-phenyl-1,2,3,4-tetrahydronaphthalen-1-yl)-2-methoxyphenyl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

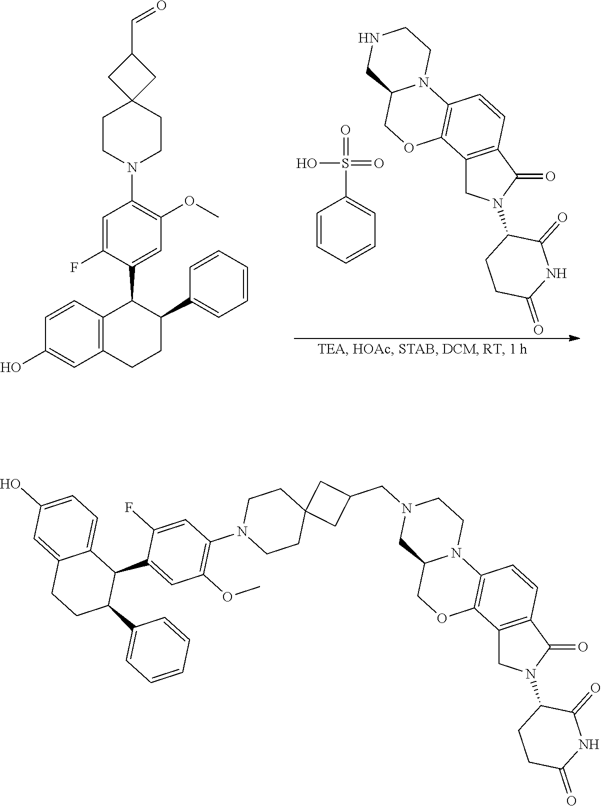

To a mixture of 7-(5-fluoro-4-((1S,2S)-6-hydroxy-2-phenyl-1,2,3,4-tetrahydronaphthalen-1-yl)-2-methoxyphenyl)-7-azaspiro[3.5]nonane-2-carbaldehyde (45.8 mg, 91.6 μmoll eq), (S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione benzenesulfonic acid (61.3 mg, 119 μmol, 1.3 eq) in DCM/DMA (2 mL) was added TEA (18.5 mg, 183 μmol, 2 eq), AcOH (16.5 mg, 275 μmol, 3 eq), sodium triacetoxyborohydride (38.8 mg, 183 μmol, 3 eq) at rt, then the mixture was stirred at rt for 1h. The resulting mixture was concentrated under reduced pressure. The residue was purified by reverse flash chromatography with the following conditions: column, C18 silica gel; mobile phase, ACN in water (0.1% FA), 0% to 50% gradient in 20 min; detector, UV 214 nm to afford (S)-3-((R)-7-((7-(5-fluoro-4-((1S,2S)-6-hydroxy-2-phenyl-1,2,3,4-tetrahydronaphthalen-1-yl)-2-methoxyphenyl)-7-azaspiro[3.5]nonan-2-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (25 mg, yield: 32.5%) as a white solid.

LC-MS purity: 100% (UV at 254 nm), 840.6 [M+H]⁺, ¹H NMR (400 MHZ, DMSO) δ 10.92 (s, 1H), 9.13 (s, 1H), 7.17 (d, J=8.2 Hz, 1H), 7.13-7.05 (m, 3H), 7.00 (d, J=8.4 Hz, 1H), 6.88-6.77 (m, 2H), 6.68-6.57 (m, 2H), 6.49 (dd, J=8.2. 2.4 Hz, 1H), 6.24 (d, J=11.6 Hz, 1H), 6.05 (d, J=7.2 Hz, 1H), 5.03 (dd, J=13.2. 4.8 Hz, 1H), 4.44 (d, J=5.2 Hz, 1H), 4.34 (dd, J=10.8. 2.4 Hz, 1H), 4.26 (d, J=16.8 Hz, 1H), 4.09 (d, J=16.8 Hz, 1H), 4.01-3.89 (m, 1H), 3.80 (d, J=11,2 Hz, 1H), 3.47 (s, 3H), 3.19-3.10 (m, 1H), 3.08-2.52 (m, 12H), 2.48-2.30 (m, 4H), 2.30-2.17 (m, 1H), 2.16-2.05 (m, 1H), 2.01-1.87 (m, 3H), 1.79-1.69 (m, 2H), 1.68-1,59 (m, 2H), 1.57-1,47 (m, 2H), 1.45-1.33 (m, 2H),

### Compound A104: (S)-N-((S)-2,6-dioxopiperidin-3-yl)-3-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-1,2,3,4,4a,5-hexahydropyrazino[1,2-d]pyrido[2,3-b][1,4]oxazine-8-carboxamide

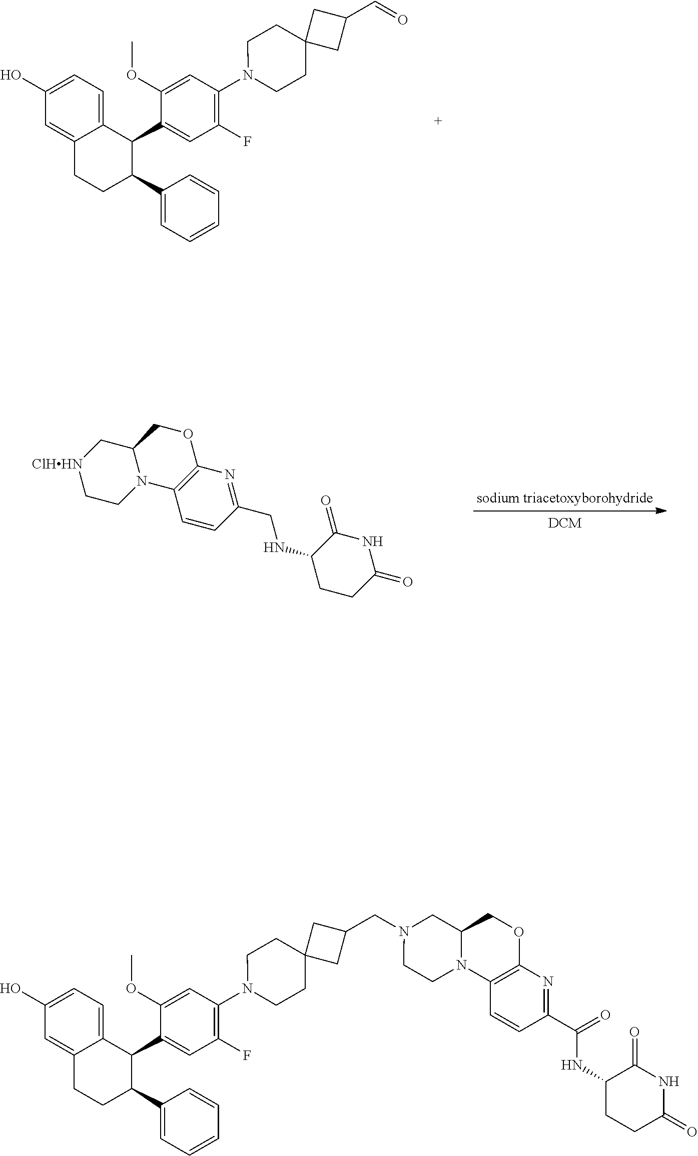

To a mixture of 7-(2-fluoro-4-((1S,2S)-6-hydroxy-2-phenyl-1,2,3,4-tetrahydronaphthalen-1-yl)-5-methoxyphenyl)-7-azaspiro[3.5]nonane-2-carbaldehyde (0.03 g, 1.0 eq, 0.06 mmol), (S)-N-((S)-2,6-dioxopiperidin-3-yl)-1,2,3,4,4a,5-hexahydropyrazino[1,2-d]pyrido[2,3-b][1,4]oxazine-8-carboxamide (0.02 g, 1,1 eq, 0.07 mmol) in DCM (3.00 mL) was added sodium triacetoxyhydroborate (0.03 g, 2.0 eq, 0.1 mmol). The resultant mixture was stirred at 25° C. for 1 h. The residue was purified by Prep-HPLC(Acetonitrile/0.05% Formate acid) to afford (S)-N-((S)-2,6-dioxopiperidin-3-yl)-3-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-1,2,3,4,4a,5-hexahydropyrazino[1,2-d]pyrido[2,3-b][1,4]oxazine-8-carboxamide (24.63 mg, 50% yield) as a white solid. LC-MS purity: 97.4% (UV at 254 nm), 829.5. [M+H]⁺.

¹H NMR (400 MHZ, DMSO) δ 10.83 (s, 1H), 9.15 (s, 1H), 8.49 (d, J=8.2 Hz, 1H), 7.56 (d, J=8.1 Hz, 1H), 7.30 (d, J=8.4 Hz, 1H), 7.16-7.00 (m, 3H), 6.75 (d, J=6.0 Hz, 2H), 6.65-6.42 (m, 3H), 6.26-6.10 (m, 2H), 4.78-4.61 (m, 2H), 4.43 (d, J=8.4 Hz, 1H), 4.13-4.03 (m, 1H), 3.76 (d, J=11.6 Hz, 1H), 3.30-3.11 (m, 4H), 3.04-2.93 (m, 5H), 2.92-2.66 (m, 10H), 2.45-2.33 (m, 2H), 2.21-2.06 (m, 3H), 2.01-1.89 (m, 3H), 1.79-1.62 (m, 4H), 1.59-1,53 (m, 2H), 1.48-1.37 (m, 2H),

### Compound A111: (R)-N-((S)-2,6-dioxopiperidin-3-yl)-3-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-1,2,3,4,4a,5-hexahydropyrazino[1,2-d]pyrido[2,3-b][1,4]oxazine-8-carboxamide

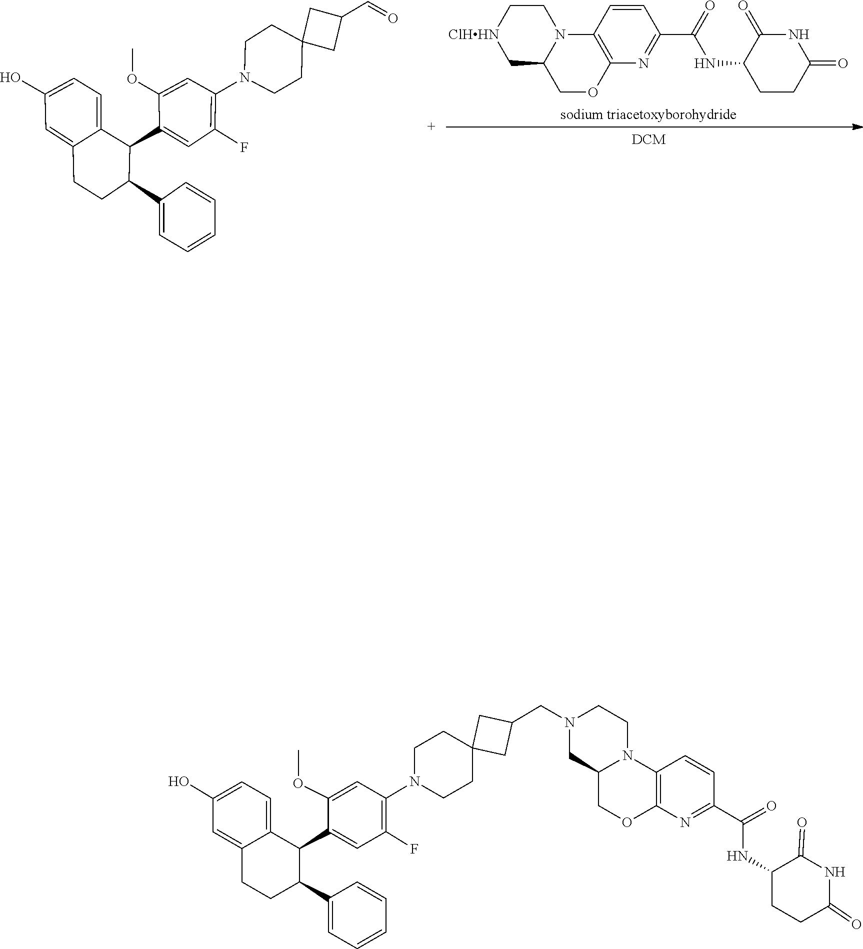

To a mixture of 7-(2-fluoro-4-((1S,2S)-6-hydroxy-2-phenyl-1,2,3,4-tetrahydronaphthalen-1-yl)-5-methoxyphenyl)-7-azaspiro[3.5]nonane-2-carbaldehyde (0.06 g, 1 eq, 0.1 mmol), (R)-N-((S)-2,6-dioxopiperidin-3-yl)-1,2,3,4,4a,5-hexahydropyrazino[1,2-d]pyrido[2,3-b][1,4]oxazine-8-carboxamide (0.05 g, 1,1 eq, 0.1 mmol) in DCM (3.00 mL) was added sodium triacetoxyhydroborate (0.05 g, 2 eq, 0.2 mmol). The resultant mixture was then stirred at 25° C. for 1 h. The residue was purified by Prep-HPLC(acetonitrile/0.05% formic acid) to afford (R)-N-((S)-2,6-dioxopiperidin-3-yl)-3-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-1,2,3,4,4a,5-hexahydropyrazino[1,2-d]pyrido[2,3-b][1,4]oxazine-8-carboxamide (57.52 mg, 60% yield) as yellow solid. LC-MS purity: 95.2% (UV at 254 nm), 829.5 [M+H]⁺.

¹H NMR (400 MHZ, DMSO) § 10.82 (s, 1H), 9.11 (s, 1H), 8.48 (d, J=8.2 Hz, 1H), 7.56 (d, J=8.0 Hz, 1H), 7.29 (d, J=8.2 Hz, 1H), 7.08 (M, 3H), 6.75 (d, J=6.8 Hz, 2H), 6.62-6.51 (m, 2H), 6.51-6.45 (m, 1H), 6.26-6.11 (m, 2H), 4.77-4.63 (m, 2H), 4.43 (d, J=10.8 Hz, 1H), 4.12-4.01 (m, 1H), 3.76 (d, J=11.6 Hz, 1H), 3.27-3.12 (m, 3H), 3.01-2.92 (m, 5H), 2.92-2.69 (m, 9H), 2.46-2.36 (m, 2H), 2.22-2.06 (m, 3H), 2.01-1.90 (m, 3H), 1.75-1,53 (m, 6H), 1.47-1.38 (m, 2H).

### Compound A119: (S)-3-((2-fluoro-4-(1-((1-(2-fluoro-4-((1S,2S)-6-hydroxy-2-phenyl-1,2,3,4-tetrahydronaphthalen-1-yl)-5-methoxyphenyl)piperidin-4-yl)methyl)piperidin-4-yl)phenyl)amino)piperidine-2,6-dione

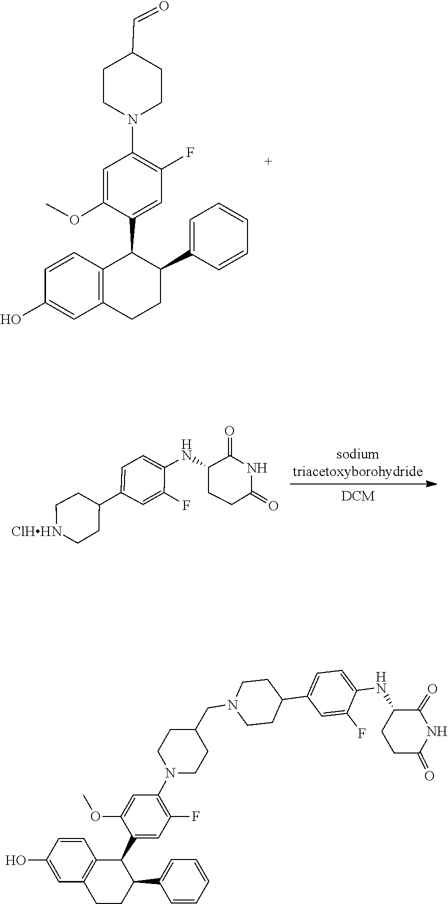

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (50.0 mg, 109 μmol, 1 eq.), (S)-3-((3-fluoro-2-(piperidin-4-yl)phenyl)amino)piperidine-2,6-dione hydrochloride (49.8 mg,, 163 μmol, 1.5 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (46 mg, 0.22 mmol, 2 eq.). The mixture was stirred at room temperature for 30 minutes and concentrated. The residue was by Prep-HPLC (acetonitrile/0.05% formic acid) to afford (S)-3-((2-fluoro-4-(1-((1-(2-fluoro-4-((1S,2S)-6-hydroxy-2-phenyl-1,2,3,4-tetrahydronaphthalen-1-yl)-5-methoxyphenyl)piperidin-4-yl)methyl)piperidin-4-yl)phenyl)amino)piperidine-2,6-dione (30.38 mg, 40.57 μmol, 37.3%) as a white solid. LC-MS purity: 95.7% (UV at 254 nm), 749.7 [M+H]⁺.

¹H NMR (400 MHZ, DMSO) δ 10.81 (s, 1H), 8.31 (s, 1H), 7.09 (d, J=6.8 Hz, 3H), 6.95-6.89 (m, 1H), 6.87-6.80 (m, 1H), 6.75 (t, J=8.8 Hz, 3H), 6.60-6.53 (m, 2H), 6.50-6.45 (m, 1H), 6.24-6.15 (m, 2H), 5.38 (d, J=6.7 Hz, 1H), 4.67 (d, J=4.8 Hz, 1H), 4.41-4.32 (m, 1H), 3.30-3,22 (m, 4H), 2.98-2.90 (m, 6H), 2.80-2,72 (m, 1H), 2.64-2.56 (m, 2H), 2.39-2.33 (m, 1H), 2.21-2.14 (m, 3H), 2.09-1.92 (m, 4H), 1.82-1.68 (m, 4H), 1.65-1,55 (m, 4H), 1.30-1,19 (m, 2H),

### Compound A120: (S)-3-((3-fluoro-4-(1-((1-(2-fluoro-4-((1S,2S)-6-hydroxy-2-phenyl-1,2,3,4-tetrahydronaphthalen-1-yl)-5-methoxyphenyl)piperidin-4-yl)methyl)piperidin-4-yl)phenyl)amino)piperidine-2,6-dione

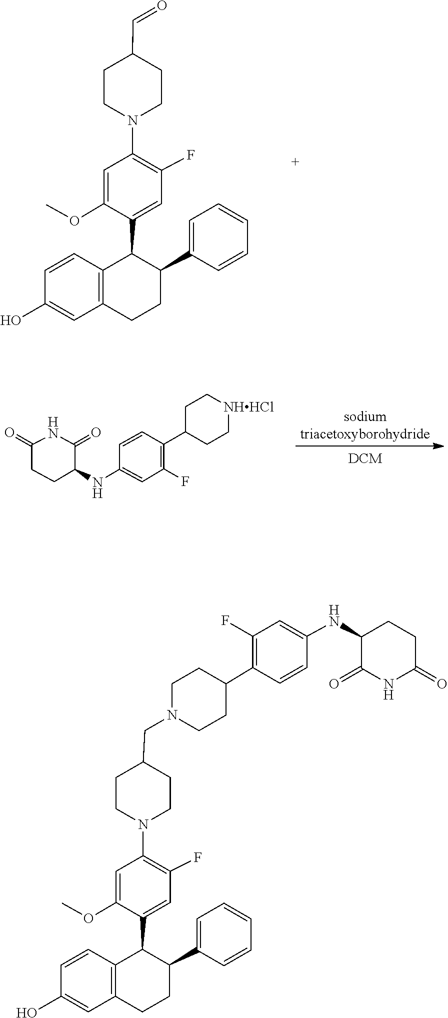

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (50.0 mg, 109 μmol, 1 eq.), (S)-3-((3-fluoro-4-(piperidin-4-yl)phenyl)amino)piperidine-2,6-dione hydrochloride (49.8 mg,, 163 μmol, 1.5 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (46 mg, 0.22 mmol, 2 eq.). The mixture was stirred at room temperature for 30 minutes and concentrated. The residue was by Prep-HPLC (acetonitrile/0.05% formic acid) to afford (S)-3-((3-fluoro-4-(1-((1-(2-fluoro-4-((1S,2S)-6-hydroxy-2-phenyl-1,2,3,4-tetrahydronaphthalen-1-yl)-5-methoxyphenyl)piperidin-4-yl)methyl)piperidin-4-yl)phenyl)amino)piperidine-2,6-dione (40.74 mg, 54,40 μmol, 50.0%) as a white solid. LC-MS purity: 92.2% (UV at 254 nm), 749.2 [M+H]⁺.

¹H NMR (400 MHZ, DMSO) δ 10.78 (s, 1H), 8.23 (s, 1H), 7.12-7.06 (m, 3H), 6.99 (t, J=8.8 Hz, 1H), 6.78-6.73 (m, 2H), 6.60-6.53 (m, 2H), 6.49-6.39 (m, 3H), 6.22-6.15 (m, 2H), 5.99 (d, J=7.6 Hz, 1H), 4.67 (d, J=5.2 Hz, 1H), 4.34-4,26 (m, 1H), 3.31-3,22 (m, 4H), 2.97-2.91 (m, 6H), 2.76-2.69 (m, 1H), 2.64-2.56 (m, 3H), 2.21-2.14 (m, 3H), 2.12-2.04 (m, 1H), 2.00-1.85 (m, 3H), 1.81-1.72 (m, 2H), 1.67-1,56 (m, 7H), 1.29-1,17 (m, 2H),

### Compound A122: (S)-3-(1′-((1-(2-fluoro-4-((1S,2S)-6-hydroxy-2-phenyl-1,2,3,4-tetrahydronaphthalen-1-yl)-5-methoxyphenyl)piperidin-4-yl)methyl)-7-oxo-5,7-dihydro-2H,6H-spiro[furo[2,3-f]isoindole-3,4′-piperidin]-6-yl)piperidine-2,6-dione

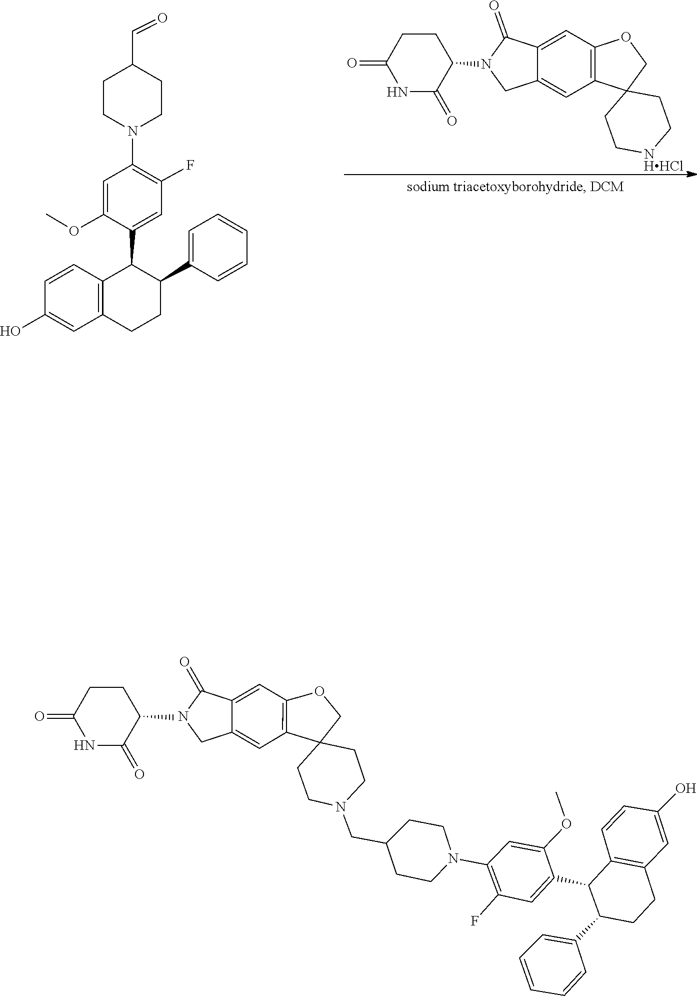

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (30.0 mg, 66.1 μmol, 1 eq.), (S)-3-(7-oxo-5,7-dihydro-2H,6H-spiro[furo[2,3-f]isoindole-3,4′-piperidin]-6-yl)piperidine-2,6-dione hydrochloride (31,1 mg,, 79.4 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was by Prep-HPLC(acetonitrile/0.05% formic acid) to afford (S)-3-(1′-((1-(2-fluoro-4-((1S,2S)-6-hydroxy-2-phenyl-1,2,3,4-tetrahydronaphthalen-1-yl)-5-methoxyphenyl)piperidin-4-yl)methyl)-7-oxo-5,7-dihydro-2H,6H-spiro[furo[2,3-f]isoindole-3,4′-piperidin]-6-yl)piperidine-2,6-dione (29.8 mg, 37.3 μmol, 57.1%) as a white solid. LC-MS purity: 92.2% (UV at 254 nm), 799.3 [M+H]⁺.

¹H NMR (400 MHZ, DMSO) δ 10.97 (s, 1H), 8.32 (s, 1H), 7.46 (s, 1H), 7.14-7.06 (m, 3H), 6.99 (d, J=9.6 Hz, 1H), 6.76 (d, J=6.2 Hz, 2H), 6.62-6.53 (m, 2H), 6.50-6.42 (m, 1H), 6.23-6.15 (m, 2H), 5.12-5.03 (m, 1H), 4.67 (d, J=5.2 Hz, 1H), 4.50-4.44 (m, 2H), 4.36-4.30 (m, 1H), 4.24-4.18 (m, 1H), 3.29-3.19 (m, 4H), 2.97-2.82 (m, 8H), 2.62-2.54 (m, 2H), 2.42-2.34 (m, 1H), 2.23-2.14 (m, 3H), 2.01-1.88 (m, 5H), 1.81-1.74 (m, 2H), 1.70-1,59 (m, 4H), 1.30-1,18 (m, 2H),

### Compound A123: (S)-3-((R)-7-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

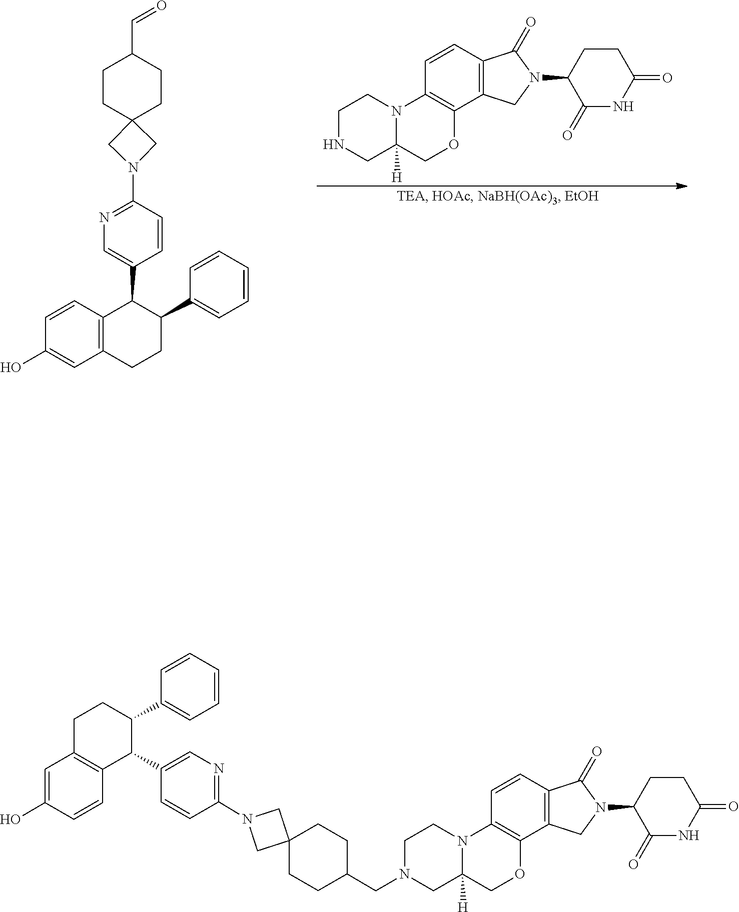 [SMILES: O=CC1CCC2(CC1)CN(c1ccc([C@@H]3c4ccc(O)cc4CC[C@@H]3c3ccccc3)cn1)C2 ; O=C1CC[C@H](N2Cc3c(ccc4c3OC[C@H]3CNCCN43)C2=O)C(=O)N1 ; O=C1CC[C@H](N2Cc3c(ccc4c3OC[C@H]3CN(CC5CCC6(CC5)CN(c5ccc([C@@H]7c8ccc(O)cc8CC[C@@H]7c7ccccc7)cn5)C6)CCN43)C2=O)C(=O)N1]

To a mixture of 2-(5-((1S,2S)-6-hydroxy-2-phenyl-1,2,3,4-tetrahydronaphthalen-1-yl)pyridin-2-yl)-2-azaspiro[3.5]nonane-7-carbaldehyde (30 mg, 0.063 mmol, 1 eq.), (S)-3-((R)-1-oxo-1,3,5,5a,6,7,8,9-octahydro-2H-pyrazino[1′,2′:4.5][1,4]oxazino[2,3-e]isoindol-2-yl)piperidine-2,6-dione (30 mg, 0.07 mmol, 1,2 eq.), and TEA (9.6 mg, 0.095 mmol, 1.5 eq.) in DCM (2.0 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was purified by reverse-phase chromatography (acetonitrile/0.05% formic acid) to afford (S)-3-((R)-7-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (12.78 mg, 39.7% yield) as white solid. LC-MS purity: 99.8% (UV at 254 nm), 793,4 [M+H]⁺.

¹H NMR (400 MHZ, DMSO) δ 10.92 (s, 1H), 9.13 (s, 1H), 7.25-7.11 (m, 4H), 7.09-6.85 (m, 4H), 6.75-6.42 (m, 4H), 6.00 (d, J=8.6 Hz, 1H), 5.12-4.91 (m, 1H), 4.47-3.74 (m, 7H), 2.99-2.86 (m, 5H), 2.80-2.54 (m, 3H), 2.47-2.36 (m, 1H), 2.20-1.94 (m, 5H), 1.87-1.62 (m, 6H), 1.57-1.38 (m, 3H), 0.98-0.84 (m, 2H),

### Compound A124: (S)-3-((S)-3-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

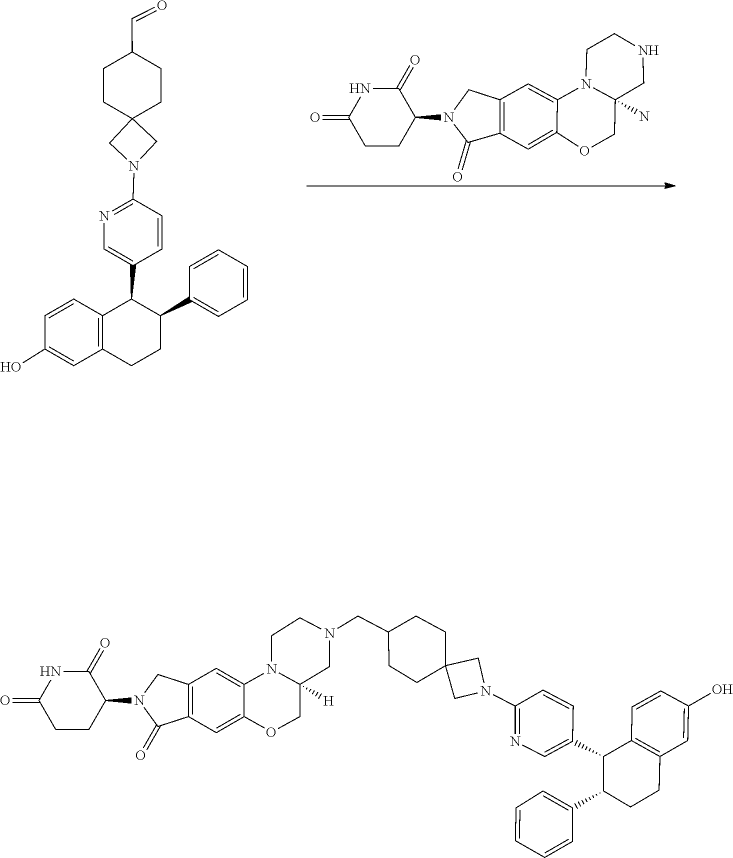

To a mixture of 2-(5-((1S,2S)-6-hydroxy-2-phenyl-1,2,3,4-tetrahydronaphthalen-1-yl)pyridin-2-yl)-2-azaspiro[3.5]nonane-7-carbaldehyde (30 mg, 0.063 mmol, 1 eq.), (S)-3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione (30 mg, 0.07 mmol, 1,2 eq.), TEA (9.6 mg, 0.095 mmol, 1.5 eq.) in DCM (2.0 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was purified by reverse-phase chromatography (acetonitrile/0.05% formic acid) to afford (S)-3-((S)-3-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-8-oxo-1,2,3,4,4a,5,8,10-octahydro-9H-pyrazino[11,2′: 4,5][1,4]oxazino[2,3-f]isoindol-9-yl)piperidine-2,6-dione (12.78 mg, 39.7% yield) as white solid. LC-MS purity: 99.8% (UV at 254 nm), 793,4 [M+H]⁺.

¹H NMR (400 MHZ, DMSO) δ 10.93 (s, 1H), 8.35 (s, 1H), 7.26-7.08 (m, 3H), 7.06-6.85 (m, 5H), 6.68-6.48 (m, 4H), 6.00 (d, J=8.6 Hz, 1H), 5.08-4.97 (m, 1H), 4.32-4.15 (m, 3H), 3.95-3.77 (m, 3H), 3.55-3,45 (m, 4H), 3.33-3.14 (m, 3H), 2.92 (d, J=11.4 Hz, 4H), 2.16-1.94 (m, 6H), 1.85-1.66 (m, 6H), 1.42 (t, J=12.4 Hz, 3H), 1.24 (s, 2H), 0.97-0.86 (m, 2H),

### Compound A127: (R)-N-((S)-2,6-dioxopiperidin-3-yl)-3-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-1,2,3,4,4a,5-hexahydropyrazino[1,2-d]pyrido[2,3-b][1,4]oxazine-8-carboxamide

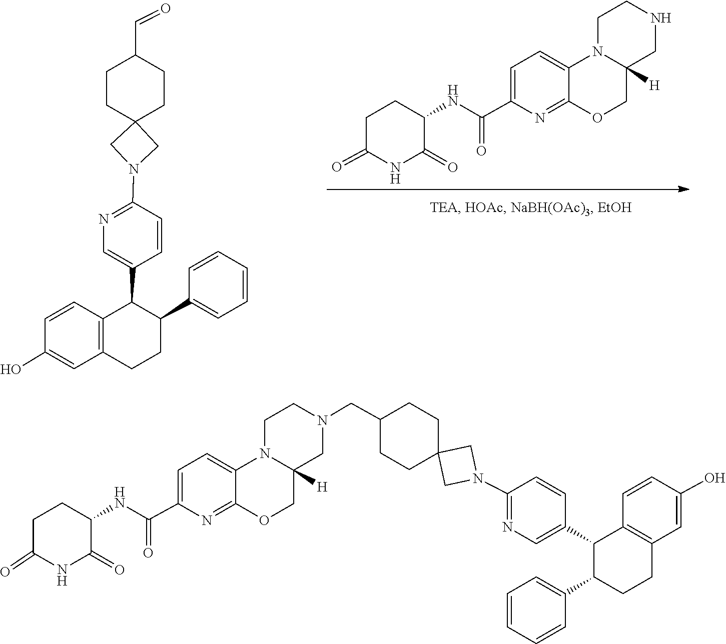 [SMILES: O=CC1CCC2(CC1)CN(c1ccc([C@@H]3c4ccc(O)cc4CC[C@@H]3c3ccccc3)cn1)C2 ; O=C1CC[C@H](NC(=O)c2ccc3c(n2)OC[C@H]2CNCCN32)C(=O)N1 ; O=C1CC[C@H](NC(=O)c2ccc3c(n2)OC[C@H]2CN(CC4CCC5(CC4)CN(c4ccc([C@@H]6c7ccc(O)cc7CC[C@@H]6c6ccccc6)cn4)C5)CCN32)C(=O)N1]

To a mixture of 2-(5-((1S,2S)-6-hydroxy-2-phenyl-1,2,3,4-tetrahydronaphthalen-1-yl)pyridin-2-yl)-2-azaspiro[3.5]nonane-7-carbaldehyde (30 mg, 0.063 mmol, 1 eq.). (R)-N-((S)-2,6-dioxopiperidin-3-yl)-1,2,3,4,4a,5-hexahydropyrazino[1,2-d]pyrido[2,3-b][1,4]oxazine-8-carboxamide (30 mg, 0.07 mmol, 1,2 eq.), TEA (9.6 mg, 0.095 mmol, 1.5 eq.) in DCM (2.0 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was purified by reverse-phase chromatography (acetonitrile/0.05% formic acid) to afford (R)-N-((S)-2,6-dioxopiperidin-3-yl)-3-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-1,2,3,4,4a,5-hexahydropyrazino[1,2-d]pyrido[2,3-b][1,4]oxazine-8-carboxamide (12.78 mg, 39.7% yield) as white solid. LC-MS purity: 99.8% (UV at 254 nm), 782.4 [M+H]⁺.

¹H NMR (400 MHZ, DMSO) δ 10.83 (s, 1H), 8.49 (d, J=8.2 Hz, 1H), 8.31 (s, 1H), 7.56 (d, J=8.2 Hz, 1H), 7.29 (d, J=8.4 Hz, 1H), 7.22-7.12 (m, 3H), 7.01 (d, J=11.8 Hz, 1H), 6.88 (d, J=7.2 Hz, 2H), 6.70-6.59 (m, 2H), 6.49 (d, J=8.2 Hz, 2H), 6.00 (d, J=8.4 Hz, 1H), 4.78-4.67 (m, 1H), 4.44 (d, J=8.6 Hz, 1H), 4.15-4.04 (m, 2H), 3.77 (d, J=10.4 Hz, 1H), 3.30 (d, J=10.8 Hz, 3H), 3.19 (s, 1H), 2.92 (d, J=10.6 Hz, 3H), 2.75 (d, J=8.8 Hz, 2H), 2.19-1.96 (m, 7H), 1.85-1.65 (m, 7H), 1.42 (t, J=11.6 Hz, 4H), 1.24 (s, 1H), 0.99-0.86 (m, 2H),

### Compound A128: (S)-3-(1′-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-7-oxo-5,7-dihydro-2H,6H-spiro[furo[2,3-f]isoindole-3,4′-piperidin]-6-yl)piperidine-2,6-dione

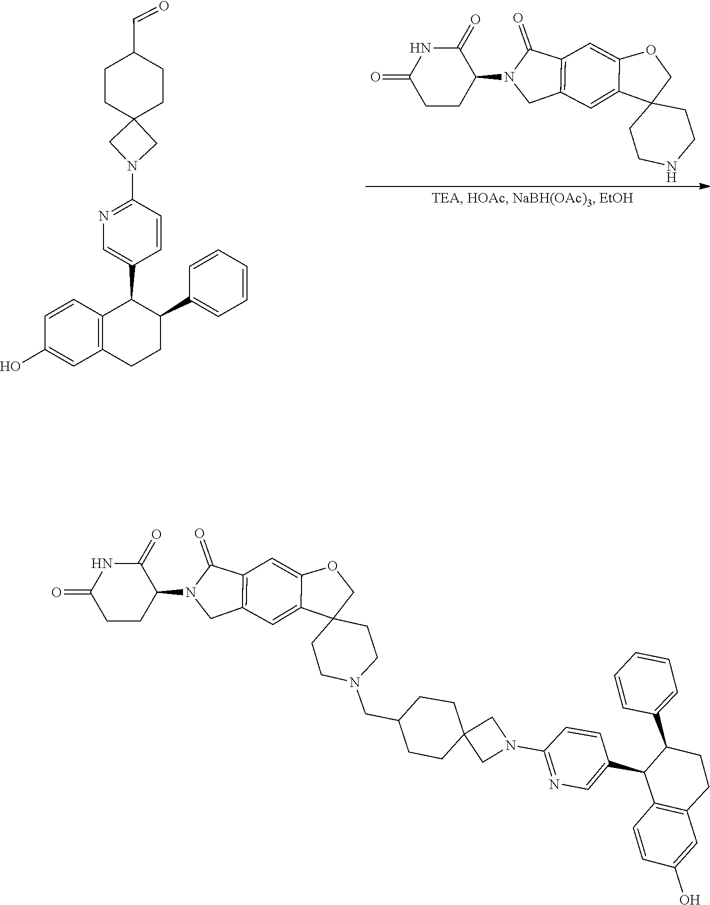 [SMILES: O=CC1CCC2(CC1)CN(c1ccc([C@@H]3c4ccc(O)cc4CC[C@@H]3c3ccccc3)cn1)C2 ; O=C1CC[C@H](N2Cc3cc4c(cc3C2=O)OCC42CCNCC2)C(=O)N1 ; O=C1CC[C@H](N2Cc3cc4c(cc3C2=O)OCC42CCN(CC3CCC4(CC3)CN(c3ccc([C@@H]5c6ccc(O)cc6CC[C@@H]5c5ccccc5)cn3)C4)CC2)C(=O)N1]

To a mixture of 2-(5-((1S,2S)-6-hydroxy-2-phenyl-1,2,3,4-tetrahydronaphthalen-1-yl)pyridin-2-yl)-2-azaspiro[3.5]nonane-7-carbaldehyde (30 mg, 0.063 mmol, 1 eq.), (S)-3-(7-oxo-5,7-dihydro-2H,6H-spiro[furo[2,3-f]isoindole-3,4′-piperidin]-6-yl)piperidine-2,6-dione (30 mg. 0.07 mmol, 1,2 eq.), TEA (9.6 mg, 0.095 mmol, 1.5 eq.) in DCM (2.0 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was purified by reverse-phase chromatography (0-50% Acetonitrile/0.05% Formic acid)) to afford (S)-3-(1′-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-7-oxo-5,7-dihydro-2H,6H-spiro[furo[2,3-f]isoindole-3,4′-piperidin]-6-yl)piperidine-2,6-dione (12.78 mg, 39.7% yield) as white solid. LC-MS purity: 99.8% (UV at 254 nm), 792.4 [M+H]⁺.

¹H NMR (400 MHZ, DMSO) δ 10.97 (s, 1H), 8.30 (s, 1H), 7.45 (s, 1H), 7.22-7.13 (m, 3H), 7.00 (s, 2H), 6.88 (d, J=7.2 Hz, 2H), 6.71-6.56 (m, 2H), 6.50 (d, J=8.4 Hz, 2H), 6.00 (d, J=8.6 Hz, 1H), 5.13-5.04 (m, 1H), 4.45 (s, 2H), 4.39-4.10 (m, 4H), 3.31 (d, J=13.0 Hz, 5H), 3.00-2,79 (m, 5H), 2.10 (d, J=6.6 Hz, 2H), 2.00-1.79 (m, 8H), 1.67 (s, 4H), 1.49-1.38 (m, 3H), 1.24 (s, 2H), 0.89 (d, J=12.2 Hz, 2H).

### Compound A129: (S)-3-(1′-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-7-oxo-5,7-dihydro-2H,6H-spiro[furo[2,3-f]isoindole-3,4′-piperidin]-6-yl)piperidine-2,6-dione

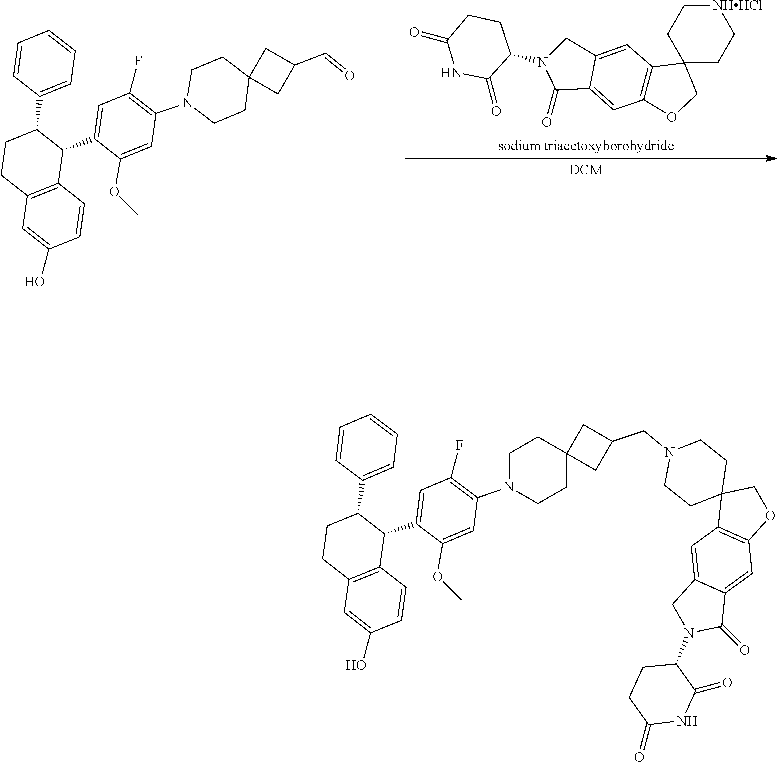

To a mixture of 7-(2-fluoro-4-((1S,2S)-6-hydroxy-2-phenyl-1,2,3,4-tetrahydronaphthalen-1-yl)-5-methoxyphenyl)-7-azaspiro[3.5]nonane-2-carbaldehyde (0.03 g, 1 eq, 0.06 mmol), (S)-3-(7-oxo-5,7-dihydro-2H,6H-spiro[furo[2,3-f]isoindole-3,4′-piperidin]-6-yl)piperidine-2,6-dione (0.02 g, 1,1 eq, 0.07 mmol) in DCM was added sodium triacetoxyborohydride (0.03 g, 0.02 mL, 2.0 eq, 0.1 mmol). The resultant mixture was then stirred at 25° C. for 1 h. The residue was purified by Prep-HPLC(acetonitrile/0.05% formic acid) to afford (S)-3-(1′-((7-(2-fluoro-4-((1S,2S)-6-hydroxy-2-phenyl-1,2,3,4-tetrahydronaphthalen-1-yl)-5-methoxyphenyl)-7-azaspiro[3.5]nonan-2-yl)methyl)-7-oxo-5,7-dihydro-2H,6H-spiro[furo[2,3-f]isoindole-3,4′-piperidin]-6-yl)piperidine-2,6-dione (27.75 mg, 60% yield) as a white solid. LC-MS purity: 97.4% (UV at 254 nm), 839.4 [M+H]⁺.

¹H NMR (400 MHZ, DMSO) δ 10.97 (s, 1H), 8.30 (s, 1H), 7.44 (s, 1H), 7.12-6.97 (m. 4H), 6.75 (d, J=6.4 Hz, 2H), 6.60-6.44 (m, 3H), 6.22-6.12 (m, 2H), 5.07 (dd, J=13.2, 4.8 Hz, 1H), 4.67 (d, J=4.8 Hz, 1H), 4.45 (s, 2H), 4.36-4.18 (m, 2H), 3.25 (d, J=9.2 Hz, 1H), 2.98-2.75 (m, 12H), 2.64-2.55 (m, 1H), 2.46-2.29 (m, 4H), 2.19-2.10 (m, 1H), 2.03-1.85 (m, 7H), 1.70-1,54 (m, 7H), 1.46-1.38 (m, 2H)

### Compound A130: (S)-3-((S)-3-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

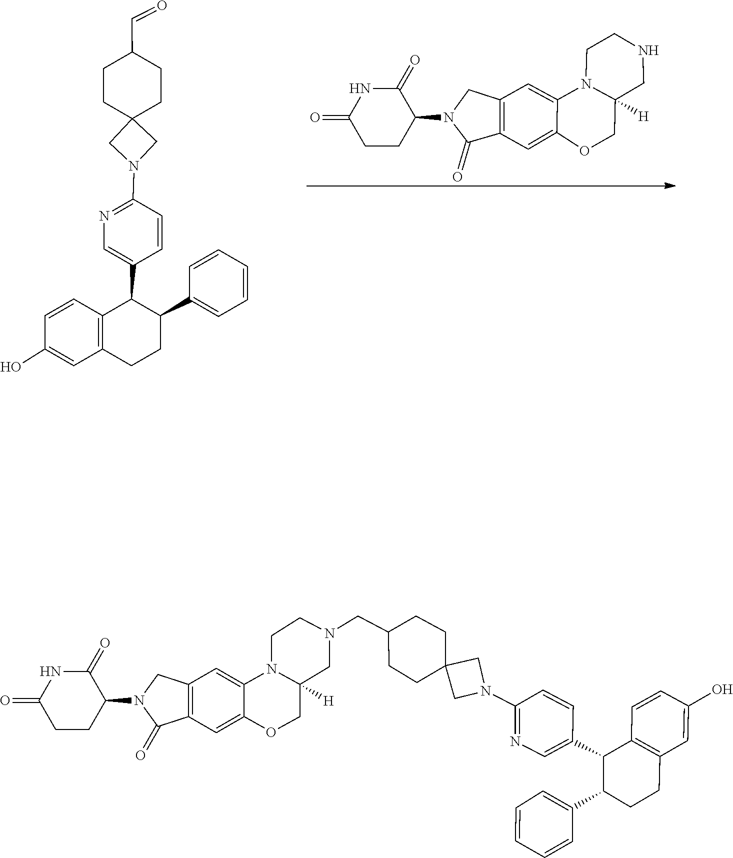 [SMILES: O=CC1CCC2(CC1)CN(c1ccc([C@@H]3c4ccc(O)cc4CC[C@@H]3c3ccccc3)cn1)C2 ; O=C1CC[C@H](N2Cc3cc4c(cc3C2=O)OC[C@@H]2CNCCN42)C(=O)N1 ; O=C1CC[C@H](N2Cc3cc4c(cc3C2=O)OC[C@@H]2CN(CC3CCC5(CC3)CN(c3ccc([C@@H]6c7ccc(O)cc7CC[C@@H]6c6ccccc6)cn3)C5)CCN42)C(=O)N1]

To a mixture of 2-(5-((1S,2S)-6-hydroxy-2-phenyl-1,2,3,4-tetrahydronaphthalen-1-yl)pyridin-2-yl)-2-azaspiro[3.5]nonane-7-carbaldehyde (30 mg, 0.063 mmol, 1 eq.), (S)-3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione (30 mg, 0.07 mmol, 1,2 eq.), TEA (9.6 mg, 0.095 mmol, 1.5 eq.) in DCM (2.0 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was purified by reverse-phase chromatography (acetonitrile/0.05% formic acid) to afford (S)-3-((S)-3-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (12.78 mg, 39.7% yield) as white solid. LC-MS purity: 99.8% (UV at 254 nm), 793,4 [M+H]⁺.

¹H NMR (400 MHZ, DMSO) δ 10.93 (s, 1H), 8.41 (s, 1H), 7.23-7.13 (m, 3H), 7.07-6.83 (m, 5H), 6.70-6.47 (m, 4H), 6.00 (d, J=8.6 Hz, 1H), 5.07-4.94 (m, 1H), 4.31-4.09 (m, 4H), 3.92-3.78 (m, 2H), 3.51 (s, 2H), 3.18 (s, 3H), 2.98-2.87 (m, 5H), 2.77-2.67 (m, 1H), 2.34 (d, J=12.0 Hz, 1H), 2.12-1.95 (m, 6H), 1.74 (d, J=11.4 Hz, 4H), 1.51-1.38 (m, 3H), 1.24 (s, 3H), 0.97-0.87 (m, 2H),

### Compound A131: (S)-3-(1′-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-6-oxo-6,8-dihydro-2H,7H-spiro[furo[2,3-e]isoindole-3,4′-piperidin]-7-yl)piperidine-2,6-dione

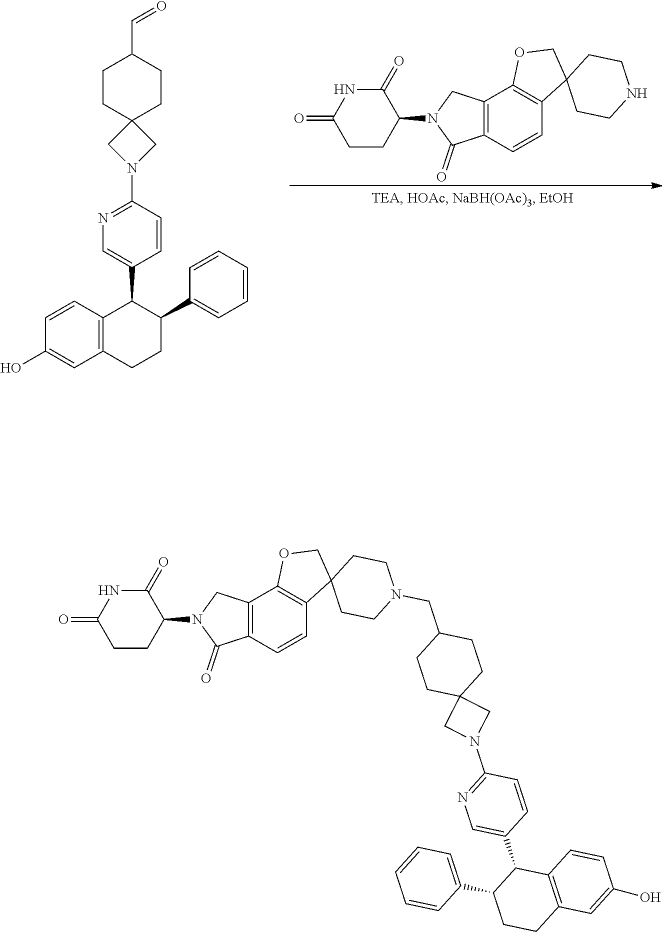 [SMILES: O=CC1CCC2(CC1)CN(c1ccc([C@@H]3c4ccc(O)cc4CC[C@@H]3c3ccccc3)cn1)C2 ; O=C1CC[C@H](N2Cc3c(ccc4c3OCC43CCNCC3)C2=O)C(=O)N1 ; O=C1CC[C@H](N2Cc3c(ccc4c3OCC43CCN(CC4CCC5(CC4)CN(c4ccc([C@@H]6c7ccc(O)cc7CC[C@@H]6c6ccccc6)cn4)C5)CC3)C2=O)C(=O)N1]

To a mixture of 2-(5-((1S,2S)-6-hydroxy-2-phenyl-1,2,3,4-tetrahydronaphthalen-1-yl)pyridin-2-yl)-2-azaspiro[3.5]nonane-7-carbaldehyde (30 mg, 0.063 mmol, 1 eq.), (S)-3-(6-oxo-6,8-dihydro-2H,7H-spiro[furo[2,3-e]isoindole-3,4′-piperidin]-7-yl)piperidine-2,6-dione (30 mg, 0.07 mmol, 1,2 eq.), TEA (9.6 mg, 0.095 mmol, 1.5 eq.) in DCM (2.0 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was purified by reverse-phase chromatography (acetonitrile/0.05% formic acid) to afford (S)-3-(1′-((2-(5-((1S,2S)-6-hydroxy-2-phenyl-1,2,3,4-tetrahydronaphthalen-1-yl)pyridin-2-yl)-2-azaspiro[3.5]nonan-7-yl)methyl)-6-oxo-6,8-dihydro-2H,7H-spiro[furo[2,3-e]isoindole-3,4′-piperidin]-7-yl)piperidine-2,6-dione (12.78 mg, 39.7% yield) as white solid. LC-MS purity: 99.8% (UV at 254 nm), 792.4 [M+H]⁺.

¹H NMR (400 MHZ, DMSO) δ 10.97 (s, 1H), 8.31 (s, 2H), 7.39 (d, J=7.4 Hz, 1H), 7.31-7.12 (m, 4H), 6.99 (s, 1H), 6.88 (d, J=7.4 Hz, 2H), 6.68-6.59 (m, 2H), 6.50 (d, J=8.2 Hz, 2H), 6.00 (d, J=8.4 Hz, 1H), 5.09-5.00 (m, 1H), 4.52 (d, J=9.6 Hz, 2H), 4.37 (d, J=11,2 Hz, 1H), 4.25-4.10 (m, 2H), 3.04-2.88 (m, 4H), 2.79 (d, J=7.0 Hz, 3H), 2.12-2.00 (m, 3H), 1.98 (d, J=6.8 Hz, 2H), 1.81-1.64 (m, 7H), 1.49-1.36 (m, 4H), 1.24 (s, 5H), 1.00-0.83 (m, 3H).

Compound A132: (S)-3-((R)-7-((7-(4-((1S,2S)-6-hydroxy-2-phenyl-1,2,3,4-tetrahydronaphthalen-1-yl)-3-methoxyphenyl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)-1-methylpiperidine-2,6-dione

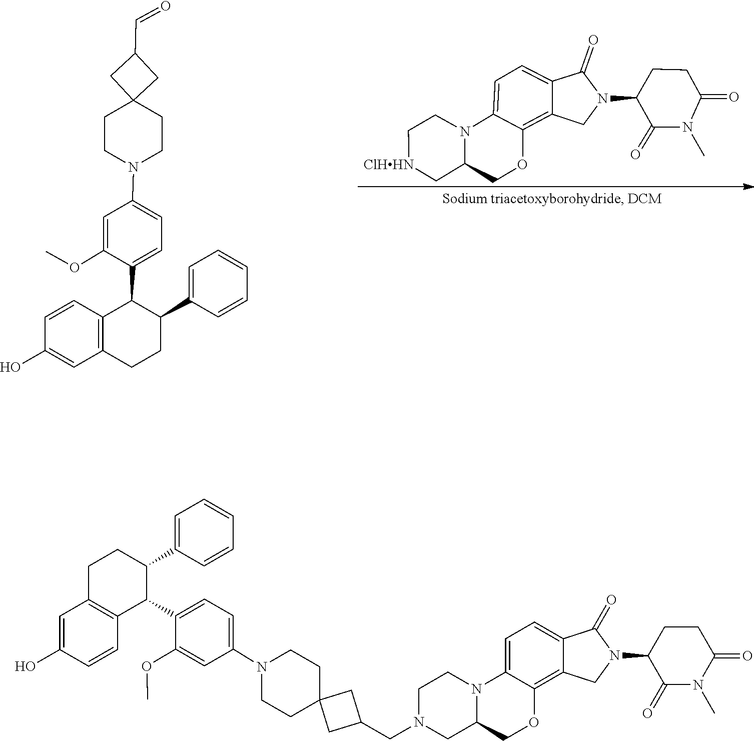

To a mixture of 7-(4-((1S,2S)-6-hydroxy-2-phenyl-1,2,3,4-tetrahydronaphthalen-1-yl)-3-methoxyphenyl)-7-azaspiro[3.5]nonane-2-carbaldehyde (30.0 mg, 66.1 μmol, 1 eq.), (S)-1-methyl-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione hydrochloride (23.7 mg,, 58.1 μmol, 1.0 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was by Prep-HPLC(acetonitrile/0.05% formic acid) to afford (S)-3-((R)-7-((7-(4-((1S,2S)-6-hydroxy-2-phenyl-1,2,3,4-tetrahydronaphthalen-1-yl)-3-methoxyphenyl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)-1-methylpiperidine-2,6-dione (26.14 mg, 31,27 μmol, 53.8%) as a white solid. LC-MS purity: 93.6% (UV at 254 nm), 836.8 [M+H]⁺.

¹H NMR (400 MHZ, DMSO) δ 8.31 (s, 1H), 7.18 (d, J=8.2 Hz, 1H), 7.09-7.05 (m, 3H), 7.01 (d, J=8.4 Hz, 1H), 6.76-6.70 (m, 2H), 6.57-6.52 (m, 2H), 6.47-6.43 (m, 1H), 6.36 (d, J=8.4 Hz, 1H), 6.32-6.27 (m, 1H), 6.10 (d, J=1.8 Hz, 1H), 5.14-5.06 (m, 1H), 4.64 (d, J=5.2 Hz, 1H), 4.37-4.30 (m, 1H), 4.29-4,21 (m, 1H), 4.15-4.06 (m, 1H), 4.00-3.93 (m, 1H), 3.83-3.79 (m, 1H), 3.22-3.14 (m, 2H), 3.02-2.97 (m, 6H), 2.94-2.88 (m, 9H), 2.77-2,70 (m, 2H), 2.45-2.37 (m, 3H), 2.18-2.08 (m, 2H), 1.97-1.91 (m, 3H), 1.79-1.70 (m, 1H), 1.66-1.57 (m, 3H), 1.54-1,48 (m, 2H), 1.46-1.38 (m, 2H),

### Compound A133: (3S)-3-[(7R)-5-[[(3R)-8-[4-[(1S,2S)-6-hydroxy-2-phenyl-tetralin-1-yl]-3-methoxy-phenyl]-1-oxa-8-azaspiro[4.5]decan-3-yl]methyl]-14-oxo-9-oxa-2,5,13-triazatetracyclo[8.7.0,02,7,011,15]heptadeca-1 (10),11 (15),16-trien-13-yl]piperidine-2,6-dione

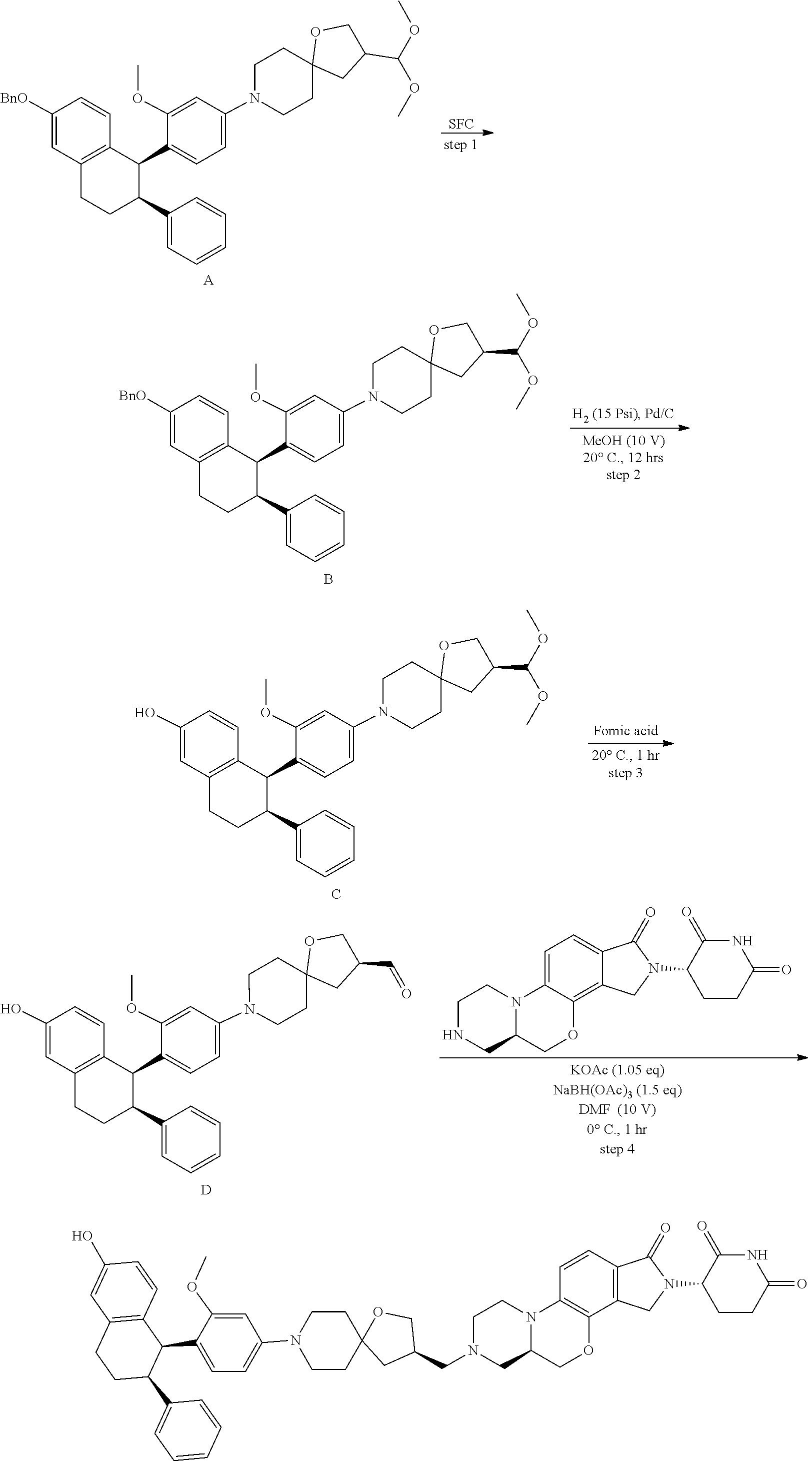

### Step 1: Synthesis of (3S)-8-[4-[(1S,2S)-6-benzyloxy-2-phenyl-tetralin-1-yl]-3-methoxy-phenyl]-3-(dimethoxymethyl)-1-oxa-8-azaspiro[4.5]decane

Compound A (350 mg) was purified by SFC(column: DAICEL CHIRALPAK AD (250 mm*30 mm, 10 um); mobile phase: [0.1% NH₃H₂O EtOH]; B %: 50%-50%, 15 min). Compound B (150 mg, 233 μmol, 84.6% yield, 98.7% purity) was obtained as a light-yellow oil.

### Step 2: Synthesis of (1S,2S)-1-[4-[(3S)-3-(dimethoxymethyl)-1-oxa-8-azaspiro[4.5]decan-8-yl]-2-methoxy-phenyl]-2-phenyl-tetralin-6-ol

The mixture of compound B (150 mg, 237 μmol, 1.00 eq), Pd/C(0.10 g, 2.37 mmol, 10% purity) in MeOH (5 mL) was degassed and purged with H₂ for 3 times, and the mixture was stirred at 20° C. for 12 hrs under H₂ atmosphere (15 Psi). The reaction mixture was filtered and concentrated under reduced pressure to give the product. Compound C(120 mg, crude) was obtained as a colorless oil.

### Step 3: Synthesis of (3S)-8-[4-[(1S,2S)-6-hydroxy-2-phenyl-tetralin-1-yl]-3-methoxy-phenyl]-1-oxa-8-azaspiro[4.5]decane-3-carbaldehyde

Compound C(60.0 mg, 110 μmol, 1.00 eq) in formic acid (1 mL) was stirred at 20° C. for 1 hr and the mixture turned to a yellow solution. Acetone (1.5 mL) was added, and the reaction mixture was concentrated under reduced pressure (no water bath) to ˜0.5 mL of solution. Compound D was obtained in a light-yellow solution and used directly in the next step, m/z (M+1): 498.

### Step 4: Synthesis of (3S)-3-[(7R)-5-[[(3R)-8-[4-[(1S,2S)-6-hydroxy-2-phenyl-tetralin-1-yl]-3-methoxy-phenyl]-1-oxa-8-azaspiro[4.5]decan-3-yl]methyl]-14-oxo-9-oxa-2,5,13-triazatetracyclo[8.7.0.02, 7.011, 15]heptadeca-1 (10), 11 (15), 16-trien-13-yl]piperidine-2,6-dione

To the suspension of compound(S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione (44.8 mg, 114 μmol, 1.05 eq. HCl) in DMF (1 mL) was added KOAc (11,2 mg, 114 μmol, 1.05 eq) in one portion at 20° C. and stirred for 10 minutes, before being cooled to 0° C. NaBH (OAc) 3 (34.5 mg, 163 μmol, 1.50 eq) and the solution of compound D (54.0 mg, 109 μmol, 1.00 eq) in formic acid (0.5 mL) was added in one portion at 0° C. The reaction mixture was stirred at 0° C. for 1 hr and to a turned brown solution. The reaction mixture was quenched by the addition of H₂O (5 mL) at 0° C., and the reaction mixture was poured into saturated NaHCO₃ solution (10 mL) at 0° C. White precipitation was observed and the mixture turned to a white suspension. The mixture was filtered to give the crude product. The crude product was purified by prep-TLC (SiO₂, EtOAc: MeOH=5:1). (3S)-3-[(7R)-5-[[(3R)-8-[4-[(1S,2S)-6-hydroxy-2-phenyl-tetralin-1-yl]-3-methoxy-phenyl]-1-oxa-8-azaspiro[4.5]decan-3-yl]methyl]-14-oxo-9-oxa-2,5,13-triazatetracyclo[8.7.0.02.7.011,15]heptadeca-1 (10),11 (15),16-trien-13-yl]piperidine-2,6-dione (90.4 mg, 107.9 μmol, 99.4% yield, 100% purity) was obtained as a white solid.

¹H NMR (400 MHZ, DMSO-d₆) δ 10.98 (s, 1H), 9.10 (s, 1H), 7.01-7.30 (m, 5H), 6.77 (br d, J=6.63 Hz, 2H), 6.57-6.65 (m, 2H), 6.50 (br d, J=7.63 Hz, 1H), 6.31-6.45 (m, 2H), 6.16 (s, 1H), 5.08 (br dd, J=13,45, 4.82 Hz, 1H), 4.69 (br d, J=4.75 Hz, 1H), 4.41 (br d, J=9.01 Hz, 1H), 4.31 (br d, J=16.76 Hz, 1H), 4.15 (br d, J=16.88 Hz, 1H), 3.81-4.07 (m, 3H), 3.46-3.53 (m, 1H), 3.09-3,29 (m, 6H), 2.87-3.08 (m, 8H), 2.78 (br d, J=9.13 Hz, 1H), 2.58-2.67 (m, 2H), 2.34-2.49 (m, 3H), 2.14-2.28 (m, 2H), 1.99 (br dd, J=11,44, 7.44 Hz, 2H), 1.56-1.84 (m, 6H), 1.40 (br dd, J=12.26, 8.38 Hz, 1H), m/z (M+1): 838.

### Compound A134: (3S)-3-[(7R)-5-[[(3S)-8-[4-[(1S,2S)-6-hydroxy-2-phenyl-tetralin-1-yl]-3-methoxy-phenyl]-1-oxa-8-azaspiro[4.5]decan-3-yl]methyl]-14-oxo-9-oxa-2,5,13-triazatetracyclo[8.7.0.02,7,011,15]heptadeca-1 (10),11 (15),16-trien-13-yl]piperidine-2,6-dione

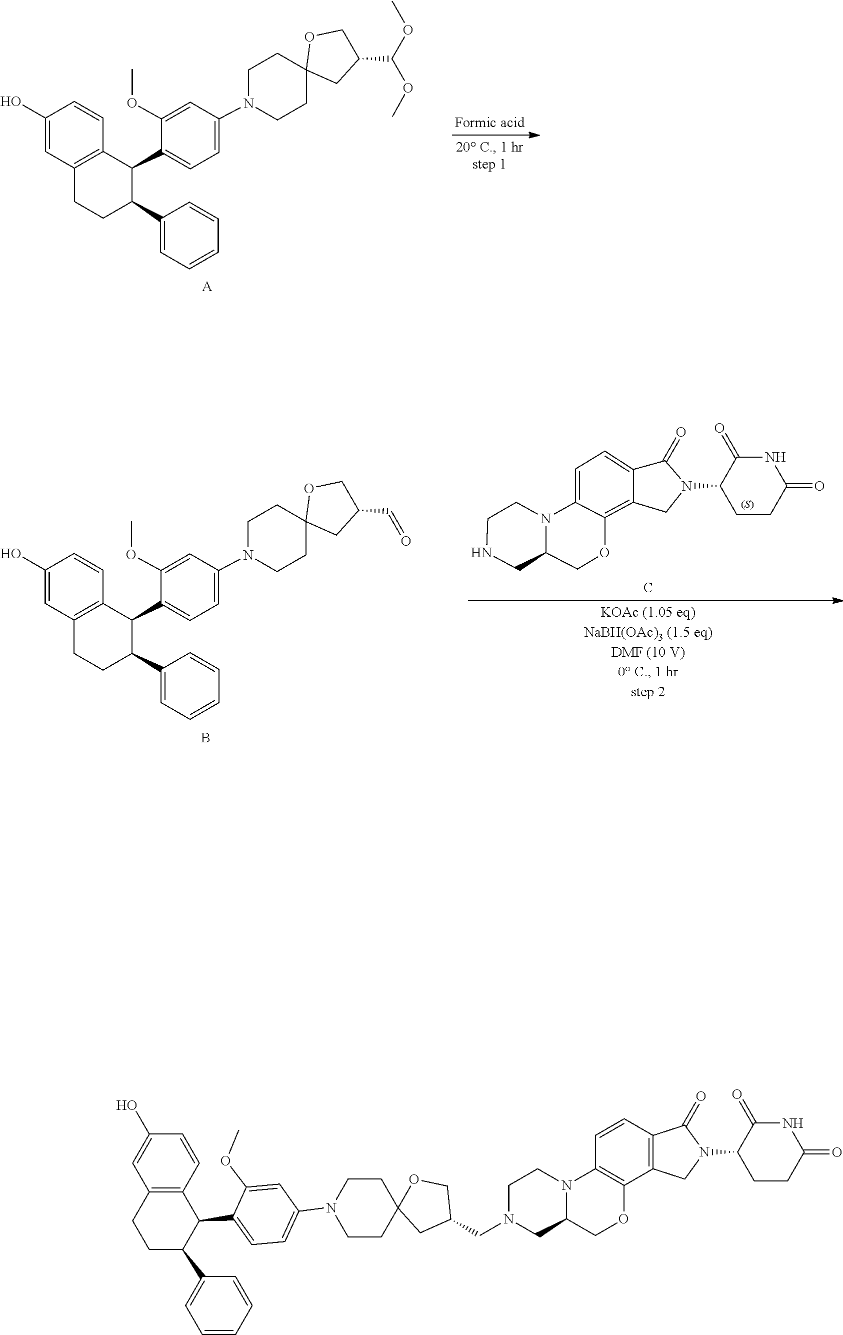

### Step 1: Synthesis of (3R)-8-[4-[(1S,2S)-6-hydroxy-2-phenyl-tetralin-1-yl]-3-methoxy-phenyl]-1-oxa-8-azaspiro[4.5]decane-3-carbaldehyde

Compound A (60.0 mg, 110 μmol, 1.00 eq) in formic acid (1 mL) was stirred at 20° C. for 1 hour and the mixture turned to a yellow solution. Acetone (1.5 mL) was added, and the reaction mixture was concentrated under reduced pressure (no water bath) to ˜0.5 mL of solution. Compound B was obtained in a light-yellow solution and used directly in the next step.

### Step 2: Synthesis of (3S)-3-[(7R)-5-[[(3S)-8-[4-[(1S,2S)-6-hydroxy-2-phenyl-tetralin-1-yl]-3-methoxy-phenyl]-1-oxa-8-azaspiro[4.5]decan-3-yl]methyl]-14-oxo-9-oxa-2.5,13-triazatetracyclo[8.7.0.02,7,011,15]heptadeca-1 (10), 11 (15), 16-trien-13-yl]piperidine-2,6-dione

To the suspension of Compound HCl of C(44.7 mg, 114 μmol, 1.05 eq, HCl) in DMF (1 mL) was added KOAc (11,2 mg, 114 μmol, 1.05 eq) in one portion at 20° C., and stirred for 10 min, before being cooled to 0° C. NaBH (OAc) 3 (34.5 mg, 163 μmol, 1.50 eq) and the solution of compound B (54.0 mg, 109 μmol, 1.00 eq) in formic acid (0.5 mL) was added in one portion at 0° C. The reaction mixture was stirred at 0° C. for 1 hr and turned to a brown solution. The reaction mixture was quenched by the addition of H₂O (5 mL) at 0° C., and the reaction mixture was poured into saturated NaHCO₃ solution (10 mL) at 0° C. White precipitate was observed and the mixture turned white suspension. The mixture was filtered to give the crude product. The residue was purified by prep-TLC (SiO₂, EtOAc: MeCN=5:1). Compound PVT-0004711 (14.8 mg, 15.2% yield, 93.5% purity) was obtained as a white solid, m/z (M+1): 838.

### Compound A135: (S)-3-((S)-3-(((R)-8-(4-((1S,2S)-6-hydroxy-2-phenyl-1,2,3,4-tetrahydronaphthalen-1-yl)-3-methoxyphenyl)-1-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

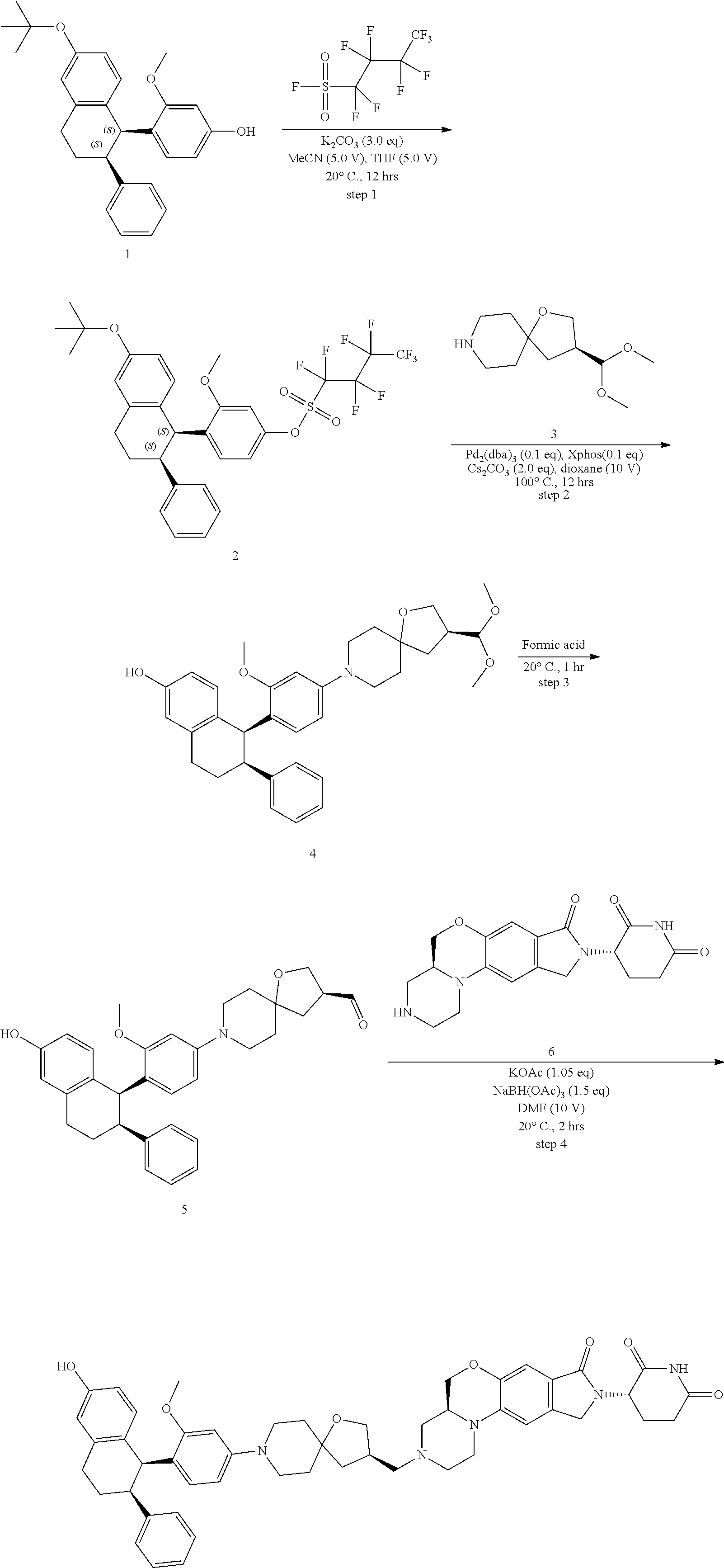

### Step 1: Synthesis of 4-((1S,2S)-6-(tert-butoxy)-2-phenyl-1,2,3,4-tetrahydronaphthalen-1-yl)-3-methoxyphenyl 1,1,2,2,3,3,4,4,4-nonafluorobutane-1-sulfonate

To a mixture of THF (15 mL) and MeCN (15 mL) was add compound 1 (1.55 g, 11,18 mmol, 3 eq) at 20° C., then 1,1,2,2,3,3,4,4,4-nonafluorobutane-1-sulfonyl fluoride (3.38 g, 11,18 mmol, 1.96 mL, 3 eq) was add to the mixture. The mixture was stirred at 20° C. for 12 hrs. TLC showed that compound 1 was consumed and the desired product was detected. The mixture was filtered, and the organic layers were concentrated under reduced pressure to give a residue. The desired product compound 2 (2.5 g, 3.65 mmol, 97.99% yield) was obtained as a white solid. Step 2: Synthesis of (5S,6S)-5-(4-((S)-3-(dimethoxymethyl)-1-oxa-8-azaspiro[4.5]decan-8-yl)-2-methoxyphenyl)-6-phenyl-5,6,7,8-tetrahydronaphthalen-2-ol

Compound 2 (2.0 g, 2.92 mmol, 1 eq) was added to dioxane (20 mL) at 20° C.; then compound 3 (943,41 mg, 4.38 mmol, 1.5 eq), Pd₂(dba) 3 (267.52 mg, 292.14 μmol, 0.1 eq), XPhos (139.27 mg, 292.14 μmol, 0.1 eq) and Cs₂CO₃ (1.90 g, 5.84 mmol, 2 eq) were added to the mixture. The mixture was stirred at 100° C. for 12 hrs. TLC showed that a little of compound 2 was remained and the desired product was detected. The mixture was filtered, and the organic layers were concentrated under reduced pressure to give a residue. The residue was purified by column chromatography on silica gel (petroleum ether/EtOAc=50:1 to 5:1). The desired product compound 4 (1,4 g, 2.33 mmol, 79.90% yield) was obtained as a yellow solid.

### Step 3: Synthesis of(S)-8-(4-((1S,2S)-6-hydroxy-2-phenyl-1,2,3,4-tetrahydronaphthalen-1-yl)-3-methoxyphenyl)-1-oxa-8-azaspiro[4.5]decane-3-carbaldehyde

A solution of compound 4 (0.7 g, 1,17 mmol, 1 eq) in formic acid (20 mL) was stirred at 20° C. for 1 hr. LCMS showed that compound 4 was consumed and the desired product was detected. The mixture was concentrated under reduced pressure to give a residue. The desired product compound 5 (0.58 g, crude) was obtained as yellow oil, m/z (M+1): 498.2

### Step 4: Synthesis of(S)-3-((S)-3-(((R)-8-(4-((1S,2S)-6-hydroxy-2-phenyl-1,2,3,4-tetrahydronaphthalen-1-yl)-3-methoxyphenyl)-1-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

To a solution of compound 6 (457 mg, 1,17 mmol, 1 eq. HCl) in DMF (2 mL) at 20° C. was add KOAc (120 mg, 1,22 mmol, 1.05 eq). The mixture was cooled to 0° C. and then NaBH (OAc) 3 (370.54 mg, 1.75 mmol, 1.5 eq) was added to the mixture. Then a solution of compound 5 (0.58 g, 1,17 mmol, 1 eq) in formic acid (0.6 mL) was added to the mixture at 0° C. The mixture was stirred at 0° C. for 5 hrs. LCMS showed that compound 5 was consumed and the desired product was detected. The mixture was quenched with water (0.2 mL). To the product was added aq.NaHCO₃ and pH was adjusted to 7. The product was extracted with EtOAc (100 mL×3). The organic layers were dried over Na₂SO₄ and concentrated under reduced pressure to give a residue. The residue was washed with MTBE/MeCN=10:1 (30 mL). The product was filtered, and the cake was concentrated under reduced pressure. (S)-3-((S)-3-(((R)-8-(4-((1S,2S)-6-hydroxy-2-phenyl-1,2,3,4-tetrahydronaphthalen-1-yl)-3-methoxyphenyl)-1-oxa-8-azaspiro[4.5]decan-3-yl)methyl)-8-oxo-1,2,3,4,4a,5,8,10-octahydro-9H-pyrazino[1′0.2′:4.5][1,4]oxazino[2,3-f]isoindol-9-yl)piperidine-2,6-dione (0.63 g, 751 μmol, 64.5% yield) was obtained as a white solid.

¹H NMR (400 MHZ, DMSO-d₆) 10.94 (s, 1H), 9.06 (s, 1H), 7.11-7.02 (m, 4H), 6.94 (s, 1H), 6.73 (br d, J=6.3 Hz, 2H), 6.60-6.53 (m, 2H), 6.46 (br d, J=8.3 Hz, 1H), 6.40-6.28 (m, 2H). 6.12 (s, 1H), 5.03 (br dd, J=4.8, 13.2 Hz, 1H), 4.65 (br d, J=4.9 Hz, 1H), 4.33-4,21 (m, 2H), 4.19-4.10 (m, 1H), 3.95-3.78 (m, 3H), 3.45 (br t, J=7.9 Hz, 1H), 3.24-3.16 (m, 2H), 3.12-3.00 (m, 5H), 2.99-2.88 (m, 7H), 2.82-2,74 (m, 1H), 2.58 (br d, J=14.8 Hz, 2H), 2.42-2.28 (m, 3H), 2.18 (br dd, J=6.5, 11.3 Hz, 1H), 2.12-2.03 (m, 1H), 2.01-1.90 (m, 2H), 1.75 (br t, J=10.9 Hz, 1H), 1.63 (br dd, J=5.0, 13.4 Hz, 5H), 1.35 (br dd, J=8.0, 12.5 Hz, 1H), m/z (M+1): 838.4

### Compound A136: (3S)-3-[(7S)-5-[[(3S)-8-[4-[(1S,2S)-6-hydroxy-2-phenyl-tetralin-1-yl]-3-methoxy-phenyl]-1-oxa-8-azaspiro[4.5]decan-3-yl]methyl]-13-oxo-9-oxa-2,5,14-triazatetracyclo[8.7.0.02,7,012,16]heptadeca-1 (17),10,12 (16)-trien-14-yl]piperidine-2,6-dione

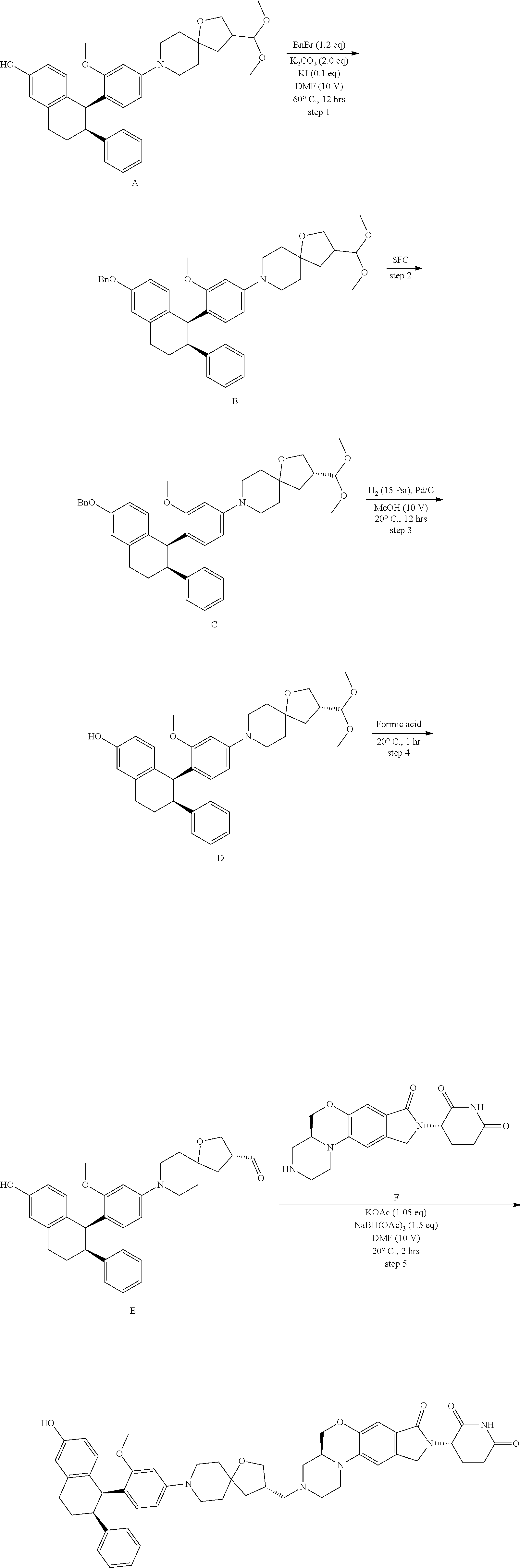

### Step 1: Synthesis of 8-[4-[(1S,2S)-6-benzyloxy-2-phenyl-tetralin-1-yl]-3-methoxy-phenyl]-3-(dimethoxymethyl)-1-oxa-8-azaspiro[4.5]decane

To a solution of compound A (370 mg, 681 μmol, 1.00 eq) in DMF (3.7 mL) was added K₂CO₃ (188 mg, 1.36 mmol, 2.00 eq), benzyl bromide (145 mg, 851 μmol, 101 μL, 1,25 eq) and KI (11.3 mg, 68.1 μmol, 0.10 eq) at 20° C. The mixture was stirred at 60° C. for 12 hours and turned yellow suspension. The reaction mixture was quenched by addition H₂O 2 mL at 20° C. and then diluted with EtOAc 5 mL and extracted with EtOAc (5 mL*4). The combined organic layers were washed with brine (10 mL*2), dried over Na₂SO₄, filtered, and concentrated under reduced pressure to give a residue. The residue was purified by prep-TLC (SiO₂, Petroleum ether: Ethyl acetate=1:1). Compound B (350 mg, 552 μmol, 81.1% yield) was obtained as a yellow oil.

### Step 2: Synthesis of (3R)-8-[4-[(1S,2S)-6-benzyloxy-2-phenyl-tetralin-1-yl]-3-methoxy-phenyl]-3-(dimethoxymethyl)-1-oxa-8-azaspiro[4.5]decane

Compound B (350 mg) was purified by SFC(column: DAICEL CHIRALPAK AD (250 mm*30 mm, 10 um); mobile phase: [0.1% NH₃H₂O ETOH]: B %: 50%-50%, 15 min). Compound C(150 mg, 236 μmol, 84.6% yield, 98.7% purity) was obtained as a light-yellow oil. Step 3: Synthesis of (1S,2S)-1-[4-[(3R)-3-(dimethoxymethyl)-1-oxa-8-azaspiro[4.5]decan-8-yl]-2-methoxy-phenyl]-2-phenyl-tetralin-6-ol

A mixture of compound C(150 mg, 237 μmol, 1.00 eq), Pd/C(0.10 g, 2.37 mmol, 10% purity) in MeOH (1 mL) was degassed and purged with H₂ for 3 times, and then the mixture was stirred at 20° C. for 12 hours under H₂ atmosphere (15 Psi). The reaction mixture was filtered and concentrated under reduced pressure to give the product. Compound D (120 mg, crude) was obtained as a colorless oil.

### Step 4: Synthesis of (3R)-8-[4-[(1S,2S)-6-hydroxy-2-phenyl-tetralin-1-yl]-3-methoxy-phenyl]-1-oxa-8-azaspiro[4.5]decane-3-carbaldehyde

Compound D (40.0 mg, 73.6 μmol, 1.00 eq) in formic acid (1 mL) was stirred at 20° C. for 1 hour and turned yellow solution. Acetone (1.5 mL) was added, and the reaction mixture was concentrated under reduced pressure (no water bath) to ˜0.5 mL of solution. Compound E was obtained in a light-yellow solution and used directly in the next step, m/z (M+1): 498.

### Step 5: Synthesis of (3S)-3-[(7S)-5-[[(3S)-8-[4-[(1S,2S)-6-hydroxy-2-phenyl-tetralin-1-yl]-3-methoxy-phenyl]-1-oxa-8-azaspiro[4.5]decan-3-yl]methyl]-13-oxo-9-oxa-2.5, 14-triazatetracyclo[8.7.0.02,7.012,16]heptadeca-1 (17), 10,12 (16)-trien-14-yl]piperidine-2,6-dione

To the suspension of compound F (29.8 mg, 75.9 μmol, 1.05 eq, HCl) in DMF (1 mL) was added KOAc (7.45 mg, 75.9 μmol, 1.05 eq) in one portion at 20° C., and stirred for 10 minutes, before cooled to 0° C. NaBH (OAc) 3 (23.0 mg, 109 μmol, 1.50 eq) and the solution of compound E (36.0 mg, 72,3 μmol, 1.00 eq) in formic acid (0.5 mL) was added in one portion at 0° C. The reaction mixture was stirred at 0° C. for 1 hour and turned brown solution. The reaction mixture was quenched by addition H₂O 5 mL at 0° C., and then the reaction mixture was poured into saturated NaHCO₃ solution (10 mL) at 0° C. White solid formed and the mixture turned white suspension. The mixture was filtered to give the crude product. The crude product was purified by prep-TLC (SiO₂, EtOAc: MeOH=5:1). (3S)-3-[(7S)-5-[[(3S)-8-[4-[(1S,2S)-6-hydroxy-2-phenyl-tetralin-1-yl]-3-methoxy-phenyl]-1-oxa-8-azaspiro[4.5]decan-3-yl]methyl]-13-oxo-9-oxa-2,5,14-triazatetracyclo[8.7.0.02,7.012,16]heptadeca-1 (17), 10,12 (16)-trien-14-yl]piperidine-2,6-dione (33,4 mg, 39.8 μmol, 55.0% yield, 100% purity) was obtained as a white solid, ¹H NMR (400 MHz, DMSO-d₆) δ 10.93 (s, 1H), 9.06 (s, 1H), 6.88-7.14 (m, 5H), 6.72 (br d, J=6.25 Hz, 2H), 6.51-6.62 (m, 2H), 6.45 (dd, J=8.25, 1.88 Hz, 1H), 6.24-6.39 (m, 2H), 6.11 (s, 1H), 5.02 (br dd, J=13.13, 5.00 Hz, 1H), 4.64 (br d, J=4.88 Hz, 1H), 4.07-4.33 (m, 3H), 3.73-3.96 (m, 3H), 3.43-3,47 (m, 1H), 3.04-3,25 (m, 7H), 2.87-3.01 (m, 7H), 2.76 (br t, J=10.69 Hz, 1H), 2.53-2.63 (m, 2H), 2.26-2.42 (m, 3H), 2.10-2.25 (m, 2H), 1.86-2.00 (m, 2H), 1.53-1.72 (m, 6H), 1.34 (br dd, J=12.63, 8.38 Hz, 1H), m/z (M+1): 838.

### Compound A137: (3S)-3-((5aR)-7-((8-(5-((1R,2R)-6-hydroxy-2-phenyl-1,2,3,4-tetrahydronaphthalen-1-yl)pyridin-2-yl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

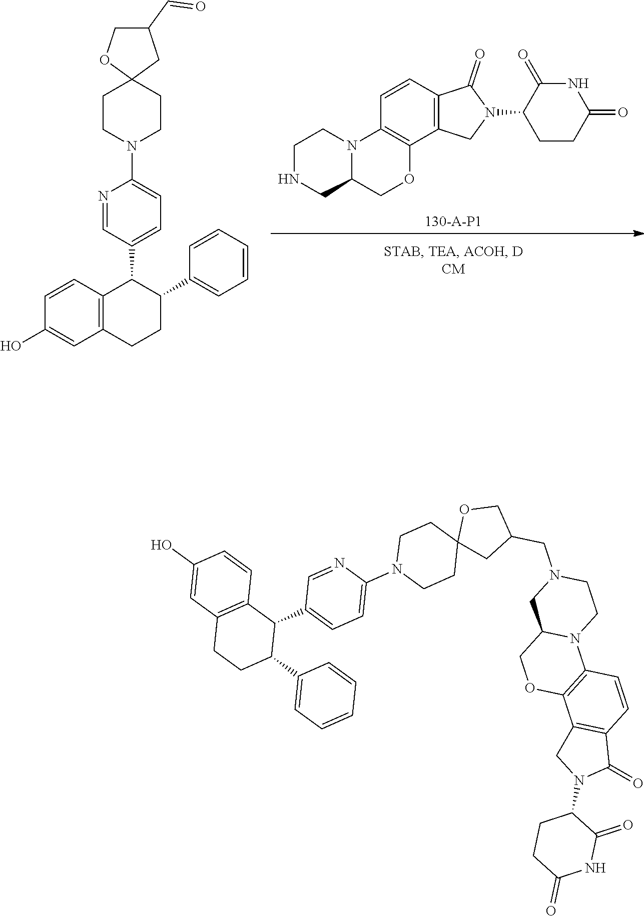 [SMILES: O=CC1COC2(CCN(c3ccc([C@H]4c5ccc(O)cc5CC[C@H]4c4ccccc4)cn3)CC2)C1 ; O=C1CC[C@H](N2Cc3c(ccc4c3OC[C@H]3CNCCN43)C2=O)C(=O)N1 ; O=C1CC[C@H](N2Cc3c(ccc4c3OC[C@H]3CN(CC5COC6(CCN(c7ccc([C@H]8c9ccc(O)cc9CC[C@H]8c8ccccc8)cn7)CC6)C5)CCN43)C2=O)C(=O)N1]

To a mixture of 8-(5-((1R,2R)-6-hydroxy-2-phenyl-1,2,3,4-tetrahydronaphthalen-1-yl)pyridin-2-yl)-1-oxa-8-azaspiro[4.5]decane-3-carbaldehyde (30 m g, 0.064 mmol 1 eq.), (S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione (25.1 mg, 0.071 mmol 1.1 eq.) TEA (9.7 mg, 0.096 mmol, 1.5 eq.) in DCM (2 mL),) was added acetic acid (6.5 mg, 0.11 mmol, 1.7 eq.) followed by sodium triacetoxyborohydride (26.2 mg, 0.12 mmol 2.0 eq.) The resultant mixture was then stirred at 25° C. for 2 h. The reaction was cooled to 20° C. and concentrated under vacuum. The residue was purified by Prep-HPLC(acetonitrile/0.05% formic acid) to afford (3S)-3-((5aR)-7-((8-(5-((1R,2R)-6-hydroxy-2-phenyl-1,2,3,4-tetrahydronaphthalen-1-yl)pyridin-2-yl)-1-oxa-8-azaspiro[4.5] decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (23.9 mg, 46.1% yield) as a white solid. LC-MS (ESI, m/z): mass calcd. For C₅₀H₅₅N₅O₆, 821.42; found, 822.4 [M+H]⁺, ¹H NMR (400 MHZ, DMSO) δ 10.94 (s, 1H), 8.31 (s, 1H), 7.22-7.10 (m, 4H), 7.05-6.97 (m, 2H), 6.88 (d, J=7.2 Hz, 2H), 6.69-6.56 (m, 2H), 6.54-6.44 (m, 3H), 5.07-4.98 (m, 1H), 4.35 (d, J=9.8 Hz, 1H), 4.30-4,20 (m, 1H), 4.13-4.05 (m, 2H), 4.00-3.77 (m, 3H), 3.53-3.39 (m, 4H), 3.22-3.06 (m, 2H), 3.04-2.84 (m, 5H), 2.77-2.66 (m, 1H), 2.63-2.52 (m, 2H), 2.45-2.25 (m, 4H), 2.15-1.90 (m, 4H), 1.81-1.62 (m, 2H), 1.59-1.39 (m, 4H), 1.38-1,24 (m, 1H),

### Compound A138: (3S)-3-((5aR)-7-((8-(5-((1S,2S)-6-hydroxy-2-phenyl-1,2,3,4-tetrahydronaphthalen-1-yl)pyridin-2-yl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

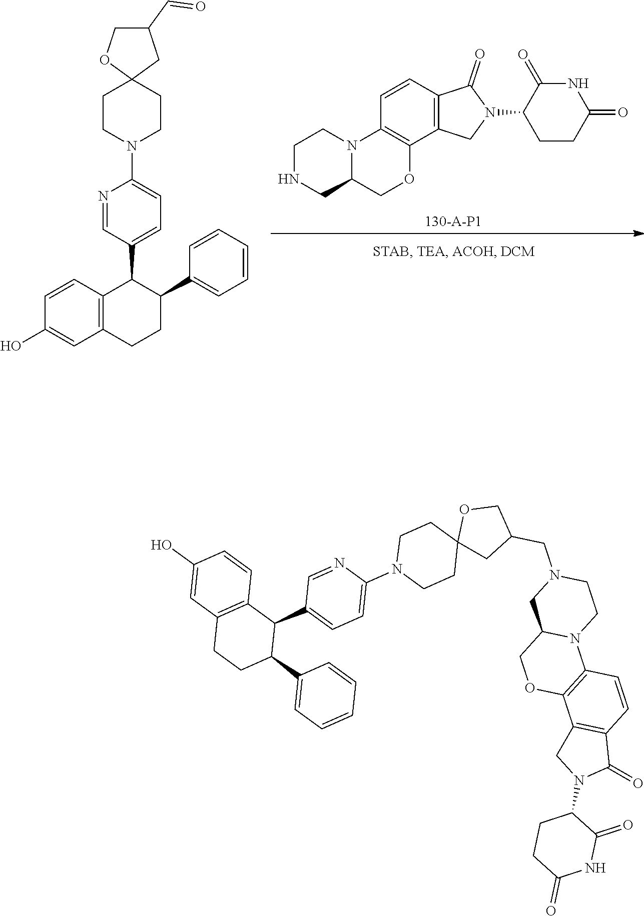 [SMILES: O=CC1COC2(CCN(c3ccc([C@@H]4c5ccc(O)cc5CC[C@@H]4c4ccccc4)cn3)CC2)C1 ; O=C1CC[C@H](N2Cc3c(ccc4c3OC[C@H]3CNCCN43)C2=O)C(=O)N1 ; O=C1CC[C@H](N2Cc3c(ccc4c3OC[C@H]3CN(CC5COC6(CCN(c7ccc([C@@H]8c9ccc(O)cc9CC[C@@H]8c8ccccc8)cn7)CC6)C5)CCN43)C2=O)C(=O)N1]

To a mixture of 8-(5-((1S,2S)-6-hydroxy-2-phenyl-1,2,3,4-tetrahydronaphthalen-1-yl)pyridin-2-yl)-1-oxa-8-azaspiro[4.5]decane-3-carbaldehyde (30 m g, 0.064 mmol 1 eq.), (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione (25.1 mg, 0.071 mmol 1.1 eq.) TEA (9.7 mg, 0.096 mmol, 1.5 eq.) in DCM (2 mL) was added acetic acid (6.5 mg, 0.11 mmol, 1.7 eq.) followed by sodium triacetoxyborohydride (26.2 mg, 0.12 mmol 2.0 eq.) The resultant mixture was then stirred at 25° C. for 2 h. The reaction was cooled to 20° C. The residue was purified by Prep-HPLC(acetonitrile/0.05% formic acid) to afford (3S)-3-((5aR)-7-((8-(5-((1S,2S)-6-hydroxy-2-phenyl-1,2,3,4-tetrahydronaphthalen-1-yl)pyridin-2-yl)-1-oxa-8-azaspiro[4.5]decan-3-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (14.78 mg, 28.7% yield) as a white solid. LC-MS (ESI, m/z): mass calcd. For C₅₀H₅₅N₅O₆, 821.42; found, 822.4 [M+H]⁺, ¹H NMR (400 MHz, DMSO) δ 10.94 (s, 1H), 8.43 (s, 1H), 7.22-7.09 (m, 4H), 7.07-6.98 (m, 2H), 6.88 (d, J=6.6 Hz, 2H), 6.67 (d, J=8.6 Hz, 1H), 6.60 (s, 1H), 6.54-6.44 (m, 3H), 5.07-4.98 (m, 1H), 4.39-4,20 (m, 2H), 4.16-4.04 (m, 2H), 4.00-3.77 (m, 3H), 3.50-3,41 (m, 4H), 3.21-3.05 (m, 2H), 3.03-2.83 (m, 5H), 2.79-2.54 (m, 2H), 2.41-2.31 (m, 4H), 2.16-1.86 (m, 5H), 1.82-1.60 (m, 3H), 1.56-1,42 (m, 4H), 1.35-1,20 (m, 2H),

### Compound A139: (S)-3-((R)-7-((7-(5-((1R,2R)-6-hydroxy-2-phenyl-1,2,3,4-tetrahydronaphthalen-1-yl)pyrimidin-2-yl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

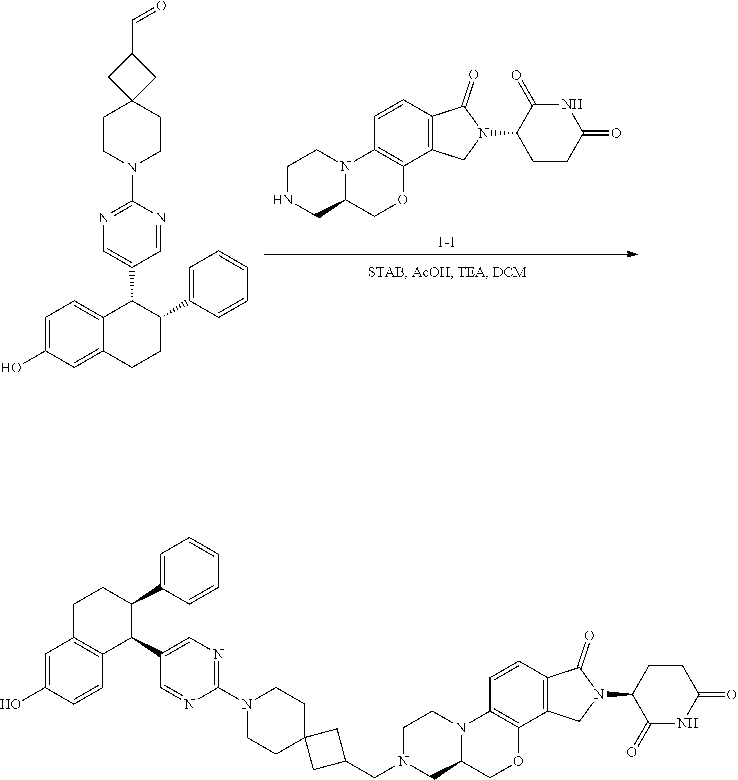 [SMILES: O=CC1CC2(CCN(c3ncc([C@H]4c5ccc(O)cc5CC[C@H]4c4ccccc4)cn3)CC2)C1 ; O=C1CC[C@H](N2Cc3c(ccc4c3OC[C@H]3CNCCN43)C2=O)C(=O)N1 ; O=C1CC[C@H](N2Cc3c(ccc4c3OC[C@H]3CN(CC5CC6(CCN(c7ncc([C@H]8c9ccc(O)cc9CC[C@H]8c8ccccc8)cn7)CC6)C5)CCN43)C2=O)C(=O)N1]

To a mixture of 7-(5-((1R,2R)-6-hydroxy-2-phenyl-1,2,3,4-tetrahydronaphthalen-1-yl)pyrimidin-2-yl)-7-azaspiro[3.5]nonane-2-carbaldehyde (25.0 mg, 1 Eq, 55.1 μmol), (S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione (23.6 mg, 1,2 Eq, 66.1 μmol), TEA (10.6 mg, 0.13 mmol, 1.5 eq.) in DCM (8.0 mL) was added acetic acid (7.6 mg, 0.11 mmol, 1.8 eq.) followed by sodium triacetoxyborohydride (23,4 mg, 16.3 μL, 2 Eq, 110 μmol). The mixture was stirred at room temperature for 50 minutes and concentrated. The residue was purified by reverse-phase chromatography (0-50% Acetonitrile/0.05% Formic acid)) to afford (S)-3-((R)-7-((7-(5-((1R,2R)-6-hydroxy-2-phenyl-1,2,3,4-tetrahydronaphthalen-1-yl)pyrimidin-2-yl)-7-azaspiro[3.5]nonan-2-yl)methyl)-1-oxo-1,3,5,5a,6,7,8,9-octahydro-2H-pyrazino[1′0.2′: 4,5][1,4]oxazino[2,3-e]isoindol-2-yl)piperidine-2,6-dione (30.71 38.68 μmol, 70.2%) as white solid.

LC-MS purity: 100% (UV at 254 nm), 794.6 [M+H]⁺.

¹H NMR (400 MHZ, DMSO) δ 10.94 (s, 1H), 8.38 (s, 1H), 7.25-7.15 (m, 6H), 6.98 (dd, J=19.6, 7.6 Hz, 3H), 6.70 (d, J=8.4 Hz, 1H), 6.60 (s, 1H), 6.52 (d, J=8.0 Hz, 1H), 5.03 (dd, J=13.2, 4.8 Hz, 1H), 4.30 (dd, J=34,4, 13.2 Hz, 2H), 4.09 (d, J=16.8 Hz, 2H), 3.95 (t, J=9.6 Hz, 1H), 3.80 (d, J=12.4 Hz, 1H), 3.55-3,43 (m, 6H), 3.24-3.08 (m, 2H), 2.96-2.85 (m, 4H), 2.75-2.56 (m, 2H), 2.42-2.37 (m, 2H), 2.16-1.86 (m, 6H), 1.75 (dd, J=26.2, 15.1 Hz, 2H), 1.43 (dd, J=31.0, 21.4 Hz, 6H),

### Compound A140: (S)-3-((R)-7-((7-(5-((1S,2S)-6-hydroxy-2-phenyl-1,2,3,4-tetrahydronaphthalen-1-yl)pyrimidin-2-yl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

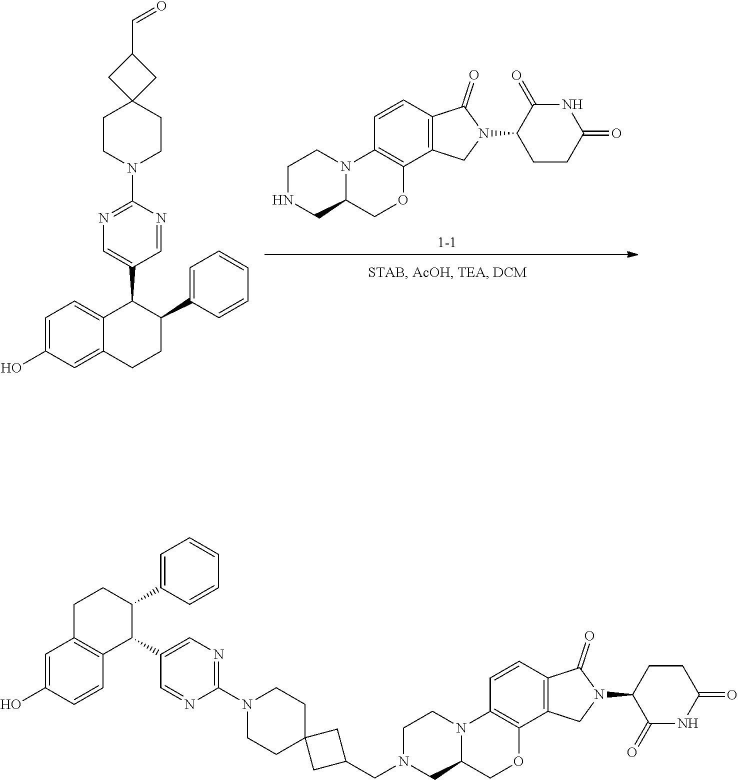 [SMILES: O=CC1CC2(CCN(c3ncc([C@@H]4c5ccc(O)cc5CC[C@@H]4c4ccccc4)cn3)CC2)C1 ; O=C1CC[C@H](N2Cc3c(ccc4c3OC[C@H]3CNCCN43)C2=O)C(=O)N1 ; O=C1CC[C@H](N2Cc3c(ccc4c3OC[C@H]3CN(CC5CC6(CCN(c7ncc([C@@H]8c9ccc(O)cc9CC[C@@H]8c8ccccc8)cn7)CC6)C5)CCN43)C2=O)C(=O)N1]

To a mixture of 7-(5-((1S,2S)-6-hydroxy-2-phenyl-1,2,3,4-tetrahydronaphthalen-1-yl)pyrimidin-2-yl)-7-azaspiro[3.5]nonane-2-carbaldehyde (25.0 mg, 1 Eq, 55.1 μmol), (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione (23.6 mg, 1,2 Eq, 66.1 μmol), TEA (10.6 mg, 0.13 mmol, 1.5 eq.) in DCM (8.0 mL) was added acetic acid (7.6 mg, 0.11 mmol, 1.8 eq.) followed by sodium triacetoxyborohydride (23,4 mg, 16.3 μL, 2 Eq, 110 μmol). The mixture was stirred at room temperature for 50 minutes and concentrated. The residue was purified by reverse-phase chromatography (acetonitrile/0.05% formic acid) to afford (S)-3-((R)-7-((7-(5-((1S,2S)-6-hydroxy-2-phenyl-1,2,3,4-tetrahydronaphthalen-1-yl)pyrimidin-2-yl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (24.66 31.06 solid, mg, μmol, 56.4%) as white LC-MS purity: 100% (UV at 254 nm), 794.5 [M+H]⁺.

¹H NMR (400 MHZ, DMSO) δ 10.94 (s, 1H), 8.31 (s, 1H), 7.25-7.16 (m, 6H), 6.97 (dd, J=19.6, 7.6 Hz, 3H), 6.70 (d, J=8.4 Hz, 1H), 6.61 (s, 1H), 6.52 (dd, J=8.4, 2.4 Hz, 1H), 5.03 (dd, J=13.2, 5.2 Hz, 1H), 4.30 (dd, J=33.6, 12.8 Hz, 2H), 4.13-4.07 (m, 2H), 3.98-3.82 (m, 2H), 3.50 (d, J=36.4 Hz, 6H), 2.94-2.84 (m, 4H), 2.68 (dd, J=34,4, 23.4 Hz, 2H), 2.42 (dd, J=26.7, 9.0 Hz, 4H), 2.13-1.69 (m, 8H), 1.43 (dd, J=30.5, 20.8 Hz, 6H),

### Compound A141: (S)-3-(1′-((7-(5-((1R,2R)-6-hydroxy-2-phenyl-1,2,3,4-tetrahydronaphthalen-1-yl)pyrimidin-2-yl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione

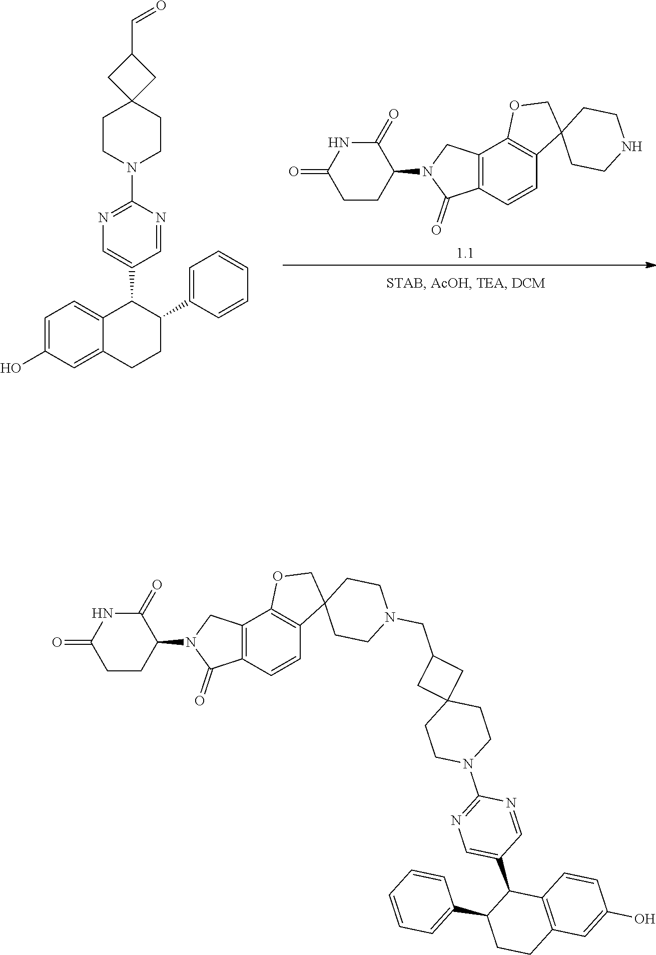 [SMILES: O=CC1CC2(CCN(c3ncc([C@H]4c5ccc(O)cc5CC[C@H]4c4ccccc4)cn3)CC2)C1 ; O=C1CC[C@H](N2Cc3c(ccc4c3OCC43CCNCC3)C2=O)C(=O)N1 ; O=C1CC[C@H](N2Cc3c(ccc4c3OCC43CCN(CC4CC5(CCN(c6ncc([C@H]7c8ccc(O)cc8CC[C@H]7c7ccccc7)cn6)CC5)C4)CC3)C2=O)C(=O)N1]

To a mixture of 7-(5-((1R,2R)-6-hydroxy-2-phenyl-1,2,3,4-tetrahydronaphthalen-1-yl)pyrimidin-2-yl)-7-azaspiro[3.5]nonane-2-carbaldehyde (25.0 mg, 1 Eq, 55.1 μmol). (S)-3-(6-oxo-6,8-dihydro-2H,7H-spiro[furo[2,3-e]isoindole-3,4′-piperidin]-7-yl)piperidine-2,6-dione (23.5 mg, 1,2 Eq, 66.1 μmol), TEA (10.6 mg, 0.13 mmol, 1.5 eq.) in DCM (8.0 mL) was added acetic acid (7.6 mg, 0.11 mmol, 1.8 eq.) followed by sodium triacetoxyhydroborate (23,4 mg, 2 Eq, 110 μmol). The mixture was stirred at room temperature for 50 minutes and concentrated. The residue was purified by reverse-phase chromatography (acetonitrile/0.05% formic acid) to afford (S) -3-(1′-((7-(5-((1R,2R)-6-hydroxy-2-phenyl-1,2,3,4-tetrahydronaphthalen-1-yl)pyrimidin-2-yl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione (24.06 mg, 30.34 μmol, 55.0%) as white solid. LC-MS purity: 100% (UV at 254 nm), 793.6 [M+H]⁺.

¹H NMR (400 MHZ, DMSO) δ 10.98 (s, 1H), 8.25 (s, 1H), 7.38 (d, J=7.6 Hz, 1H), 7.21 (dt, J=14.8, 8.2 Hz, 6H), 6.95 (d, J=7.2 Hz, 2H), 6.70 (d, J=8.4 Hz, 1H), 6.60 (s, 1H), 6.52 (d. J=8.0 Hz, 1H), 5.08 (dd, J=13.2, 4.8 Hz, 1H), 4.51 (s, 2H), 4.37 (d, J=17.2 Hz, 1H), 4.20 (d, J=17.2 Hz, 1H), 4.11 (d, J=4.8 Hz, 1H), 3.56-3.44 (m, 6H), 3.04-2.66 (m, 6H), 2.46-2.32 (m, 4H), 2.02-1.85 (m, 8H), 1.65 (t, J=10.4 Hz, 2H), 1.52-1.34 (m, 6H).

### Compound A142: (S)-3-(1′-((7-(5-((1S,2S)-6-hydroxy-2-phenyl-1,2,3,4-tetrahydronaphthalen-1-yl)pyrimidin-2-yl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione

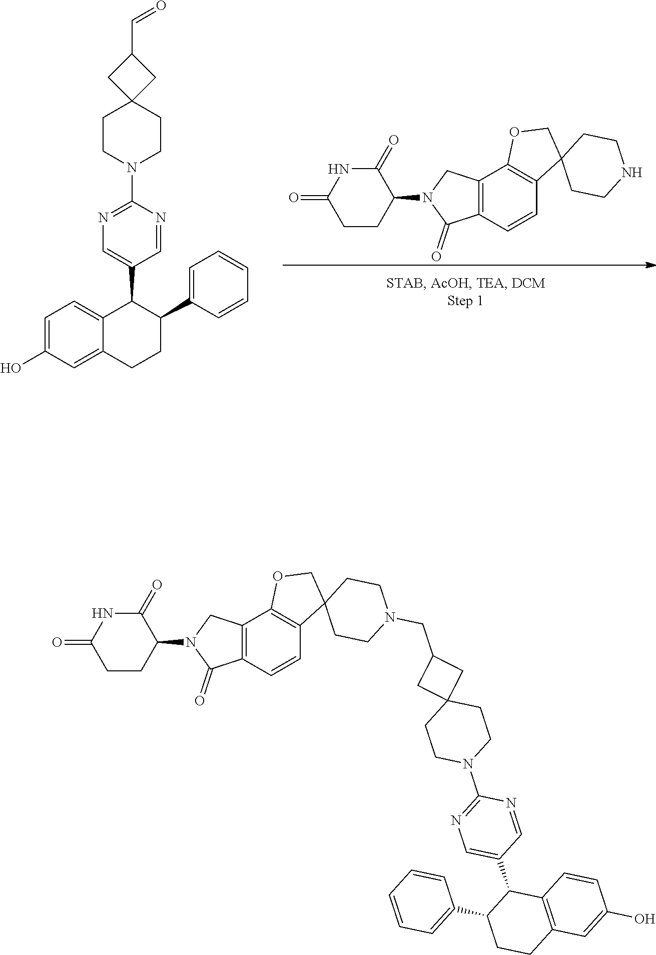 [SMILES: O=CC1CC2(CCN(c3ncc([C@@H]4c5ccc(O)cc5CC[C@@H]4c4ccccc4)cn3)CC2)C1 ; O=C1CC[C@H](N2Cc3c(ccc4c3OCC43CCNCC3)C2=O)C(=O)N1 ; O=C1CC[C@H](N2Cc3c(ccc4c3OCC43CCN(CC4CC5(CCN(c6ncc([C@@H]7c8ccc(O)cc8CC[C@@H]7c7ccccc7)cn6)CC5)C4)CC3)C2=O)C(=O)N1]

To a mixture of 7-(5-((1S,2S)-6-hydroxy-2-phenyl-1,2,3,4-tetrahydronaphthalen-1-yl)pyrimidin-2-yl)-7-azaspiro[3.5]nonane-2-carbaldehyde (25.0 mg, 1 Eq, 55.1 μmol), (S)-3-(6-oxo-6,8-dihydro-2H,7H-spiro[furo[2,3-e]isoindole-3,4′-piperidin]-7-yl)piperidine-2,6-dione (23.5 mg, 1,2 Eq, 66.1 μmol), TEA (10.6 mg, 0.13 mmol, 1.5 eq.) in DCM (8.0 mL) was added acetic acid (7.6 mg, 0.11 mmol, 1.8 eq.) followed by Sodium triacetoxyborohydride (23,4 mg, 16.3 μL, 2 Eq, 110 μmol). The mixture was stirred at room temperature for 50 minutes and concentrated. The residue was purified by reverse-phase chromatography (acetonitrile/0.05% formic acid) to afford (S)-3-(1′-((7-(5-((1S,2S)-6-hydroxy-2-phenyl-1,2,3,4-tetrahydronaphthalen-1-yl)pyrimidin-2-yl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione (32.89 mg, 41,48 μmol, 75.3%) as white solid.

LC-MS purity: 100% (UV at 254 nm), 793.6 [M+H]⁺.

¹H NMR (400 MHZ, DMSO) δ 10.94 (s, 1H), 8.38 (s, 1H), 7.25-7.15 (m, 6H), 6.98 (dd, J=19.6. 7.6 Hz, 3H), 6.70 (d, J=8.4 Hz, 1H), 6.60 (s, 1H), 6.52 (d, J=8.0 Hz, 1H), 5.03 (dd, J=13.2, 5.2 Hz, 1H), 4.30 (dd, J=34.0, 13.2 Hz, 2H), 4.09 (d, J=16.8 Hz, 2H), 3.95 (t, J=9.6 Hz, 1H), 3.83-3.77 (m, 1H), 3.56-3.47 (m, 4H), 3.37-3.31 (m, 2H), 3.15 (s, 2H), 2.94-2.83 (m, 4H), 2.75-2.56 (m, 2H), 2.45-2.36 (m, 3H), 2.18-1.85 (m, 6H), 1.82-1.69 (m, 2H), 1.51-1.34 (m, 6H),

### Compound A143: (S)-3-((S)-3-((7-(5-((1R,2R)-6-hydroxy-2-phenyl-1,2,3,4-tetrahydronaphthalen-1-yl)pyrimidin-2-yl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

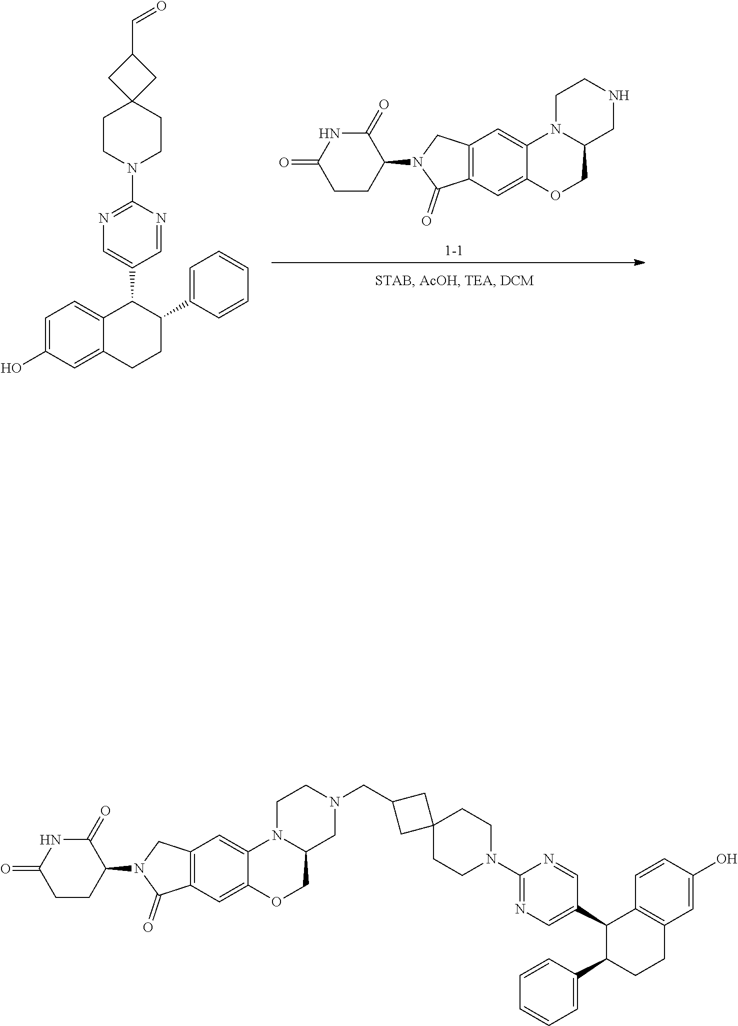 [SMILES: O=CC1CC2(CCN(c3ncc([C@H]4c5ccc(O)cc5CC[C@H]4c4ccccc4)cn3)CC2)C1 ; O=C1CC[C@H](N2Cc3cc4c(cc3C2=O)OC[C@@H]2CNCCN42)C(=O)N1 ; O=C1CC[C@H](N2Cc3cc4c(cc3C2=O)OC[C@@H]2CN(CC3CC5(CCN(c6ncc([C@H]7c8ccc(O)cc8CC[C@H]7c7ccccc7)cn6)CC5)C3)CCN42)C(=O)N1]

To a mixture of 7-(5-((1R,2R)-6-hydroxy-2-phenyl-1,2,3,4-tetrahydronaphthalen-1-yl)pyrimidin-2-yl)-7-azaspiro[3.5]nonane-2-carbaldehyde (25.0 mg, 1 Eq, 55.1 μmol), (S)-3-((S)-8-oxo-1,2,3,4,4a,5,8,10-octahydro-9H-pyrazino[11,2′: 4,5][1,4]oxazino[2,3-f]isoindol-9-yl)piperidine-2,6-dione (23.6 mg, 1,2 Eq, 66.1 μmol), TEA (10.6 mg, 0.13 mmol, 1.5 eq.) in DCM (8.0 mL) was added acetic acid (7.6 mg, 0.11 mmol, 1.8 eq.) followed by sodium triacetoxyhydroborate (23,4 mg, 2 Eq, 110 μmol). The mixture was stirred at room temperature for 50 minutes and concentrated. The residue was purified by reverse-phase chromatography (acetonitrile/0.05% formic acid) to afford (S)-3-((S)-3-((7-(5-((1R,2R)-6-hydroxy-2-phenyl-1,2,3,4-tetrahydronaphthalen-1-yl)pyrimidin-2-yl)-7-azaspiro[3.5]nonan-2-yl)methyl)-8-oxo-1,2,3,4,4a,5,8,10-octahydro-9H-pyrazino[11,2′: 4,5][1,4]oxazino[2,3-f]isoindol-9-yl)piperidine-2,6-dione (22.97 mg, 28.93 solid. μmol, 52.5%) as white

LC-MS purity: 100% (UV at 254 nm), 794.5 [M+H]⁺.

¹H NMR (400 MHZ, DMSO) δ 10.94 (s, 1H), 7.26-7.16 (m, 5H), 7.03 (s, 1H), 6.98-6.92 (m, 3H), 6.70 (d, J=8.4 Hz, 1H), 6.61 (s, 1H), 6.52 (d, J=8.0 Hz, 1H), 5.02 (dd, J=13.2, 4.8 Hz, 1H), 4.38-4.04 (m, 5H), 3.94-3.74 (m, 3H), 3.14 (s, 2H), 3.04-2.82 (m, 6H), 2.78-2.54 (m. 3H), 2.47-2.29 (m, 4H), 2.14-1.68 (m, 8H), 1.53-1.35 (m, 6H),

### Compound A144: (S)-3-((S)-3-((7-(5-((1S,2S)-6-hydroxy-2-phenyl-1,2,3,4-tetrahydronaphthalen-1-yl)pyrimidin-2-yl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

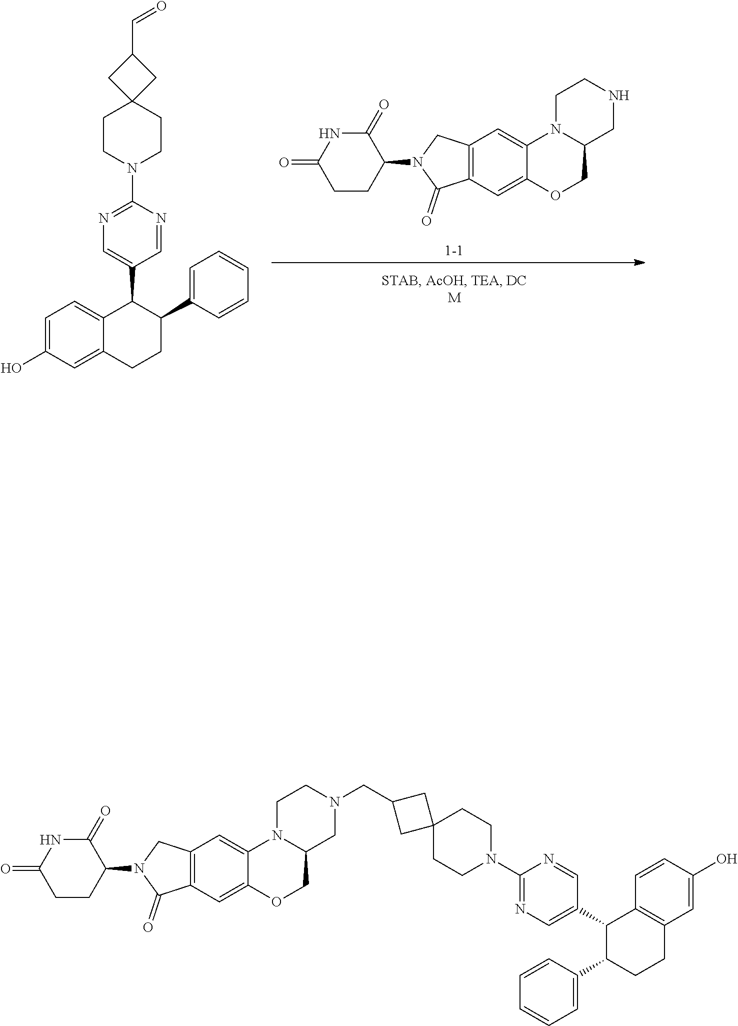 [SMILES: O=CC1CC2(CCN(c3ncc([C@@H]4c5ccc(O)cc5CC[C@@H]4c4ccccc4)cn3)CC2)C1 ; O=C1CC[C@H](N2Cc3cc4c(cc3C2=O)OC[C@@H]2CNCCN42)C(=O)N1 ; O=C1CC[C@H](N2Cc3cc4c(cc3C2=O)OC[C@@H]2CN(CC3CC5(CCN(c6ncc([C@@H]7c8ccc(O)cc8CC[C@@H]7c7ccccc7)cn6)CC5)C3)CCN42)C(=O)N1]

To a mixture of 7-(5-((1S,2S)-6-hydroxy-2-phenyl-1,2,3,4-tetrahydronaphthalen-1-yl)pyrimidin-2-yl)-7-azaspiro[3.5]nonane-2-carbaldehyde (25.0 mg, 1 Eq, 55.1 μmol), (S)-3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione (19.6 mg, 1 Eq, 55.1 μmol), TEA (10.6 mg, 0.13 mmol, 1.5 eq.) in DCM (8.0 mL) was added acetic acid (7.6 mg, 0.11 mmol, 1.8 eq.) followed by sodium triacetoxyhydroborate (23,4 mg, 2 Eq, 110 μmol). The mixture was stirred at room temperature for 50 minutes and concentrated. The residue was purified by reverse-phase chromatography (0 acetonitrile/0.05% formic acid) to afford (S)-3-((S)-3-((7-(5-((1S,2S)-6-hydroxy-2-phenyl-1,2,3,4-tetrahydronaphthalen-1-yl)pyrimidin-2-yl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)pipcridinc-2,6-dione (26.58 mg, 33,48 μmol, 60.7%) as white solid.

LC-MS purity: 100% (UV at 254 nm), 794.7 [M+H]⁺.

¹H NMR (400 MHZ, DMSO) δ 10.93 (s, 1H), 9.18 (s, 1H), 7.26-7.16 (m, 5H), 7.03 (s, 1H), 6.98-6.91 (m, 3H), 6.70 (d, J=8.4 Hz, 1H), 6.61 (d, J=2.4 Hz, 1H), 6.52 (dd, J=8.4, 2.4 Hz, 1H), 5.02 (dd, J=13.2, 4.8 Hz, 1H), 4.29-4.09 (m, 4H), 3.92-3.86 (m, 1H), 3.78 (d, J=11,2 Hz, 1H), 3.58-3.43 (m, 6H), 3.24-3.05 (m, 2H), 2.99-2.84 (m, 5H), 2.78-2.69 (m, 1H), 2.44-2.29 (m, 3H), 2.13-1.91 (m, 5H), 1.82-1.66 (m, 2H), 1.51-1.34 (m, 6H).

Compound A145: (S)-3-((R)-7-((7-(2-fluoro-4-((1R,2S)-6-hydroxy-2-phenyl-1,2,3,4-tetrahydronaphthalen-1-yl)phenyl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

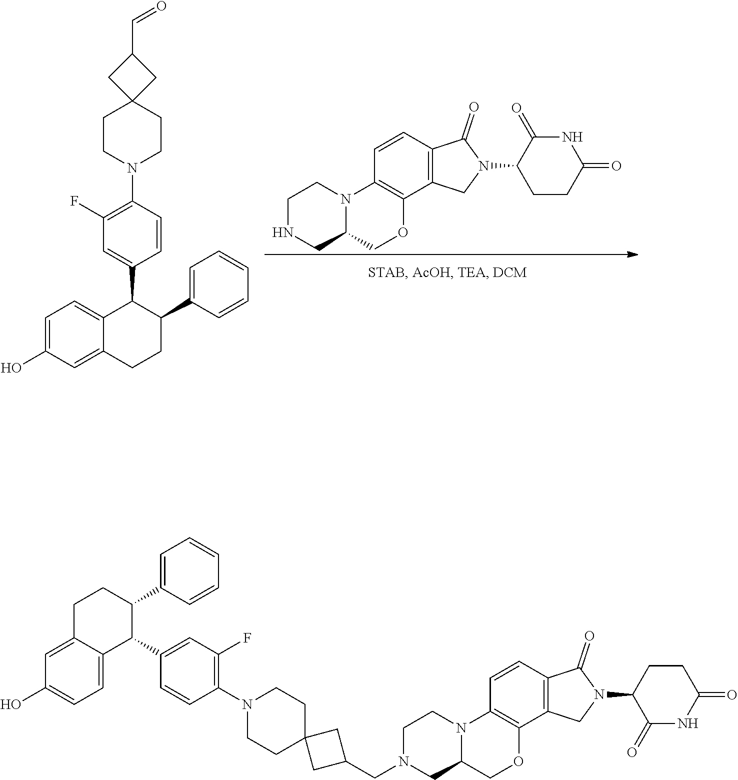

To a mixture of 7-(2-fluoro-4-((1R,2S)-6-hydroxy-2-phenyl-1,2,3,4-tetrahydronaphthalen-1-yl)phenyl)-7-azaspiro[3.5]nonane-2-carbaldehyde (28.0 mg, 1 Eq, 59.6 μmol), (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione (25.5 mg, 1,2 Eq, 71.6 μmol), TEA (10.6 mg, 0.13 mmol, 1.5 eq.) in DCM (8.0 mL) was added acetic acid (7.6 mg, 0.11 mmol, 1.8 eq.) followed by sodium triacetoxyhydroborate (25.3 mg, 2 Eq, 119 μmol). The mixture was stirred at room temperature for 50 minutes and concentrated. The residue was purified by reverse-phase chromatography (0-50% Acetonitrile/0.05% Formic acid)) to afford (S)-3-((R)-7-((7-(2-fluoro-4-((1R,2S)-6-hydroxy-2-phenyl-1,2,3,4-tetrahydronaphthalen-1-yl)phenyl)-7-azaspiro[3.5]nonan-2-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (40.82 mg, 50.40 μmol, 84.5%) as white solid.

LC-MS purity: 100% (UV at 254 nm),810.4 [M+H]⁺.

¹H NMR (400 MHZ, DMSO) δ 10.94 (s, 1H), 8.26 (s, 1H), 7.20-6.98 (m, 5H), 6.86-6.78 (m, 2H), 6.61 (d, J=8.8 Hz, 2H), 6.55-6.42 (m, 3H), 6.25 (d, J=13.6 Hz, 1H), 5.02 (dd, J=13.2, 5.2 Hz, 1H), 4.47-4,22 (m, 3H), 4.10 (d, J=16.8 Hz, 1H), 3.98-3.91 (m, 1H), 3.80 (d, J=11,2 Hz, 1H), 3.22-3.10 (m, 2H), 3.03-2.84 (m, 9H), 2.76-2.58 (m, 2H), 2.49-2.33 (m, 4H), 2.22-2.06 (m, 2H), 1.99-1.86 (m, 3H), 1.76-1,57 (m, 4H), 1.52-1.37 (m, 4H),

### Compound A146: (S)-3-(1′-((7-(2-fluoro-4-((1R,2S)-6-hydroxy-2-phenyl-1,2,3,4-tetrahydronaphthalen-1-yl)phenyl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione

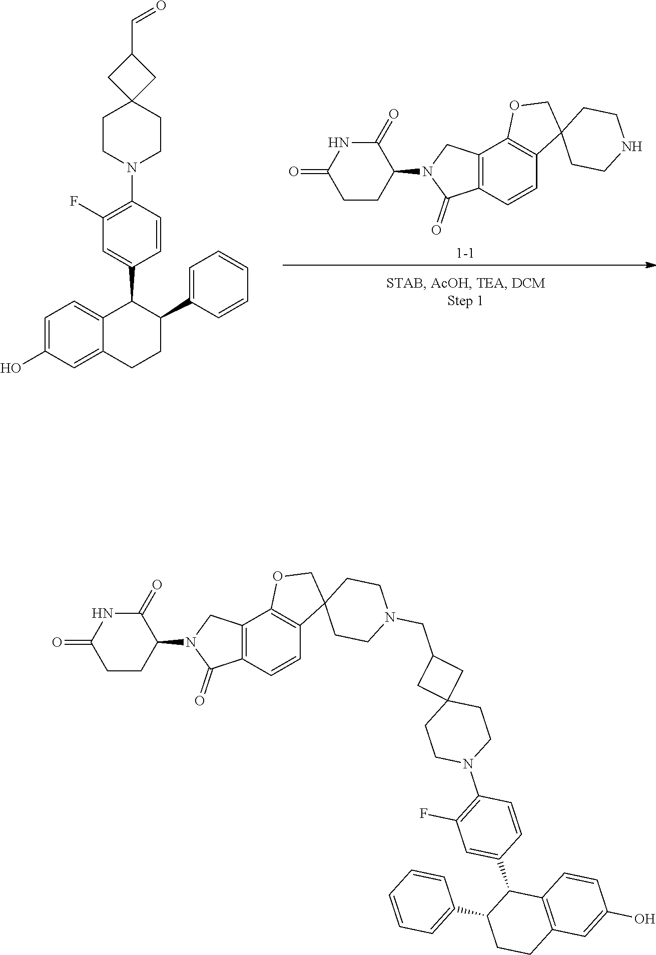 [SMILES: O=CC1CC2(CCN(c3ccc([C@@H]4c5ccc(O)cc5CC[C@@H]4c4ccccc4)cc3F)CC2)C1 ; O=C1CC[C@H](N2Cc3c(ccc4c3OCC43CCNCC3)C2=O)C(=O)N1 ; O=C1CC[C@H](N2Cc3c(ccc4c3OCC43CCN(CC4CC5(CCN(c6ccc([C@@H]7c8ccc(O)cc8CC[C@@H]7c7ccccc7)cc6F)CC5)C4)CC3)C2=O)C(=O)N1]

To a mixture of 7-(2-fluoro-4-((1R,2S)-6-hydroxy-2-phenyl-1,2,3,4-tetrahydronaphthalen-1-yl)phenyl)-7-azaspiro[3.5]nonane-2-carbaldehyde (30.0 mg, 1 Eq, 63.9 μmol), (S)-3-(6-oxo-6,8-dihydro-2H,7H-spiro[furo[2,3-e]isoindole-3,4′-piperidin]-7-yl)piperidine-2,6-dione (27.2 mg. 1,2 Eq, 76.7 μmol), TEA (10.6 mg, 0.13 mmol, 1.5 eq.) in DCM (8.0 mL) was added acetic acid (7.6 mg, 0.11 mmol, 1.8 eq.) followed by sodium triacetoxyhydroborate (27.1 mg, 2 Eq, 128 μmol). The mixture was stirred at room temperature for 50 minutes and concentrated. The residue was purified by reverse-phase chromatography (acetonitrile/0.05% formic acid) to afford (S)-3-(1′-((7-(2-fluoro-4-((1R,2S)-6-hydroxy-2-phenyl-1,2,3,4-tetrahydronaphthalen-1-yl)phenyl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione (47.69 mg, 58.95 μmol, 92,3%) as white solid. LC-MS purity: 99.5% (UV at 254 nm), 809.8 [M+H]⁺.

¹H NMR (400 MHZ, DMSO) δ 10.98 (s, 1H), 8.31 (s, 1H), 7.32 (dd, J=47.6, 7.6 Hz, 2H), 7.13-7.07 (m, 3H), 6.85-6.79 (m, 2H), 6.63-6.45 (m, 5H), 6.25 (d, J=12.8 Hz, 1H), 5.08 (dd, J=13.2, 4.8 Hz, 1H), 4.57-4.33 (m, 4H), 4.21 (d, J=17.2 Hz, 1H), 3.05-2.85 (m, 8H), 2.65-2.53 (m, 2H), 2.47-2.33 (m, 4H), 2.10-1.81 (m, 8H), 1.74-1.36 (m, 10H),

### Compound A147: (S)-3-((R)-7-((7-(2-fluoro-4-((1S,2R)-6-hydroxy-2-phenyl-1,2,3,4-tetrahydronaphthalen-1-yl)phenyl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

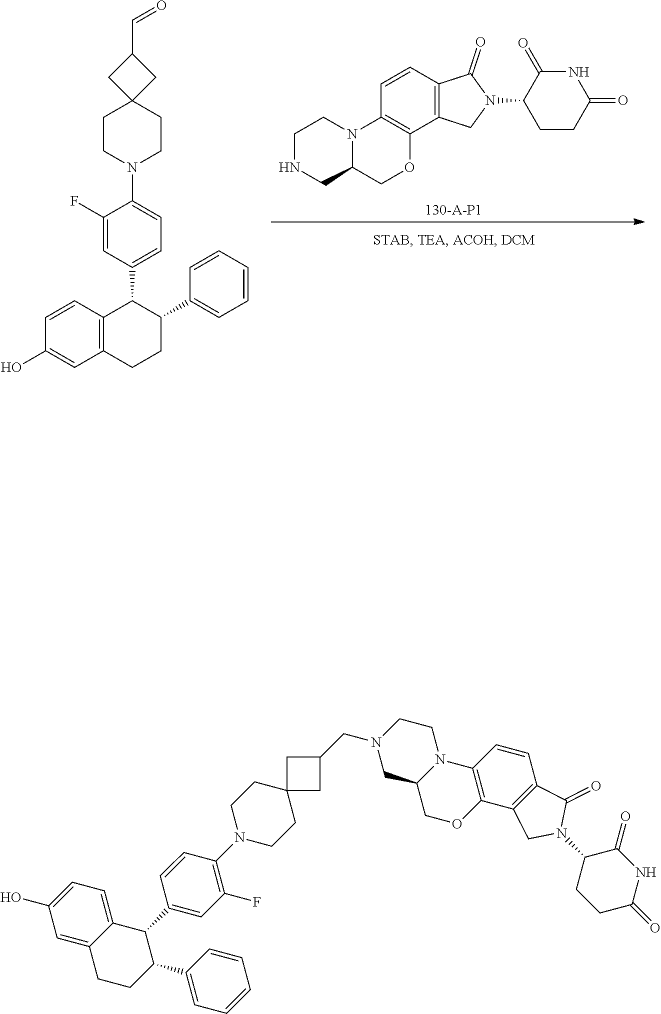 [SMILES: O=CC1CC2(CCN(c3ccc([C@H]4c5ccc(O)cc5CC[C@H]4c4ccccc4)cc3F)CC2)C1 ; O=C1CC[C@H](N2Cc3c(ccc4c3OC[C@H]3CNCCN43)C2=O)C(=O)N1 ; O=C1CC[C@H](N2Cc3c(ccc4c3OC[C@H]3CN(CC5CC6(CCN(c7ccc([C@H]8c9ccc(O)cc9CC[C@H]8c8ccccc8)cc7F)CC6)C5)CCN43)C2=O)C(=O)N1]

To a mixture of 7-(2-fluoro-4-((1S,2R)-6-hydroxy-2-phenyl-1,2,3,4-tetrahydronaphthalen-1-yl)phenyl)-7-azaspiro[3.5]nonane-2-carbaldehyde (33.5 m g, 0.071 mmol 1 eq.), (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione (27.72 mg, 0.078 mmol 1.1 eq.) TEA (10.7 mg, 0.106 mmol, 1.5 eq.) in DCM (2 mL),) was added acetic acid (7.2 mg, 0.12 mmol, 1.7 eq.) followed by sodium triacetoxyborohydride (30.0 mg, 0.14 mmol 2.0 eq.) The resultant mixture was then stirred at 25° C. for 2 h. The reaction was cooled to 20° C. and concentrated under vacuum. The residue was purified by Prep-HPLC(acetonitrile/0.05% formic acid) to afford (S)-3-((R)-7-((7-(2-fluoro-4-((1S,2R)-6-hydroxy-2-phenyl-1,2,3,4-tetrahydronaphthalen-1-yl)phenyl)-7-azaspiro[3.5]nonan-2-yl)methyl)-1-oxo-1,3,5,5a,6,7,8,9-octahydro-2H-pyrazino[1′0.2′: 4,5][1,4]oxazino[2,3-e]isoindol-2-yl)piperidine-2,6-dione (32.01 mg, 55.9% yield) as a white solid. LC-MS (ESI, m/z): mass calcd. For C₅₀H₅₅N₅O₆, 821.42; found, 822.4 [M+H]⁺, ¹H NMR (400 MHZ, DMSO) δ 10.93 (s, 1H), 9.14 (s, 1H), 7.17 (d, J=8.2 Hz, 1H), 7.11-7.07 (m, 3H), 7.00 (d, J=8.5 Hz, 1H), 6.84-6.78 (m, 2H), 6.64-6.56 (m, 2H), 6.55-6.41 (m, 3H), 6.25 (d, J=13.6 Hz, 1H), 5.07-4.99 (m, 1H), 4.47-4.40 (m, 1H), 4.38-4.30 (m, 1H), 4.30-4,20 (m, 1H), 4.13-4.04 (m, 1H), 4.00-3.89 (m, 1H), 3.84-3.76 (m, 1H), 3.19-3.09 (m, 2H), 3.06-2.82 (m, 10H), 2.75-2.67 (m, 1H), 2.63-2.54 (m, 1H), 2.43-2.36 (m, 3H), 2.22-2.05 (m, 2H), 1.98-1.88 (m, 3H), 1.79-1,58 (m, 4H), 1.52-1.38 (m, 4H).

Compound A148: (S)-3-(1′-((7-(2-fluoro-4-((1S,2R)-6-hydroxy-2-phenyl-1,2,3,4-tetrahydronaphthalen-1-yl)phenyl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione

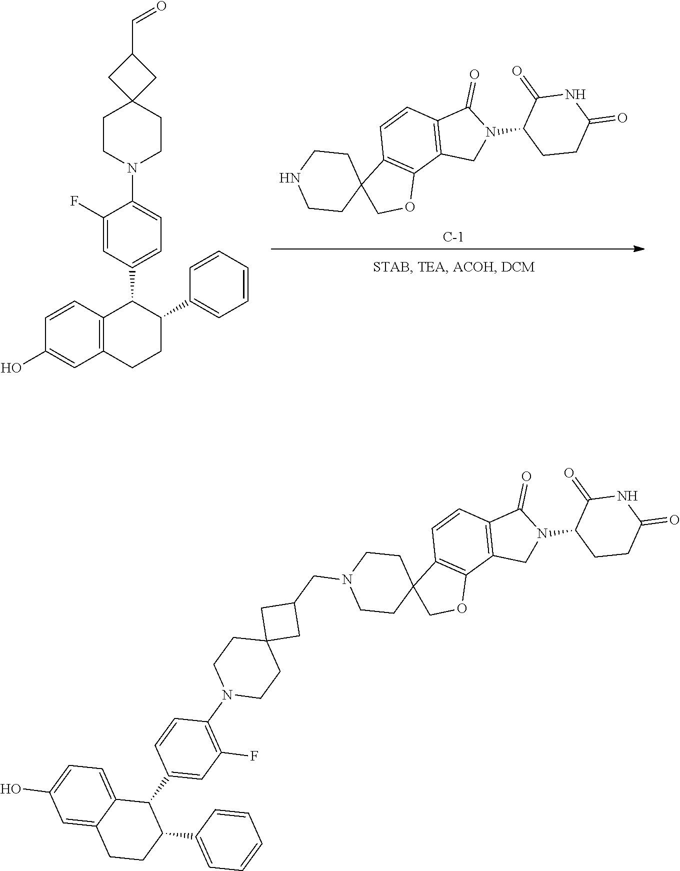

To a mixture of 7-(2-fluoro-4-((1S,2R)-6-hydroxy-2-phenyl-1,2,3,4-tetrahydronaphthalen-1-yl)phenyl)-7-azaspiro[3.5]nonane-2-carbaldehyde (28.2 mg, 0.060 mmol 1 eq.), (S)-3-(6-oxo-6,8-dihydro-2H,7H-spiro[furo[2,3-e]isoindole-3,4′-piperidin]-7-yl)piperidine-2,6-dione (23,43 mg, 0.066 mmol 1.1 eq.) TEA (9.1 mg, 0.09 mmol, 1.5 eq.) in DCM (2 mL) was added acetic acid (6.12 mg, 0.10 mmol, 1.7 eq.) followed by sodium triacetoxyborohydride (25.44 mg,0.12 mmol 2.0 eq.) The resultant mixture was then stirred at 25° C. for 2 h. The reaction was cooled to 20° C. and concentrated under vacuum. The residue was purified by Prep-HPLC(acetonitrile/0.05% formic acid) to afford (S)-3-(1′-((7-(2-fluoro-4-((1S,2R)-6-hydroxy-2-phenyl-1,2,3,4-tetrahydronaphthalen-1-yl)phenyl)-7-azaspiro[3.5]nonan-2-yl)methyl)-6-oxo-6,8-dihydro-2H,7H-spiro[furo[2,3-e]isoindole-3,4′-piperidin]-7-yl)piperidine-2,6-dione (28.81 mg, 59.4% yield) as a white solid. LC-MS (ESI, m/z): mass calcd. For C₅₀H₅₅N₅O₆, 821.42; found, 822.4 [M+H]⁺, ¹H NMR (400 MHZ, DMSO) δ 10.97 (s, 1H), 9.13 (s, 1H), 7.38 (d, J=7.4 Hz, 1H), 7.26 (d, J=7.6 Hz, 1H), 7.13-7.06 (m, 3H), 6.84-6.79 (m, 2H), 6.64-6.57 (m, 2H), 6.55-6.42 (m, 3H), 6.26 (d, J=13.6 Hz, 1H), 5.12-5.04 (m, 1H), 4.56-4.48 (m, 2H), 4.47-4.42 (m, 1H), 4.41-4.33 (m, 1H), 4.24-4.16 (m, 1H), 3.04-2.80 (m, 10H), 2.68-2.55 (m, 2H), 2.47-2.36 (m, 3H), 2.23-2.13 (m, 1H), 2.09-1.83 (m, 7H), 1.74-1,56 (m, 5H), 1.54-1.36 (m, 4H),

### Compound A149: (S)-3-((S)-3-((7-(2-fluoro-4-((1S,2R)-6-hydroxy-2-phenyl-1,2,3,4-tetrahydronaphthalen-1-yl)phenyl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

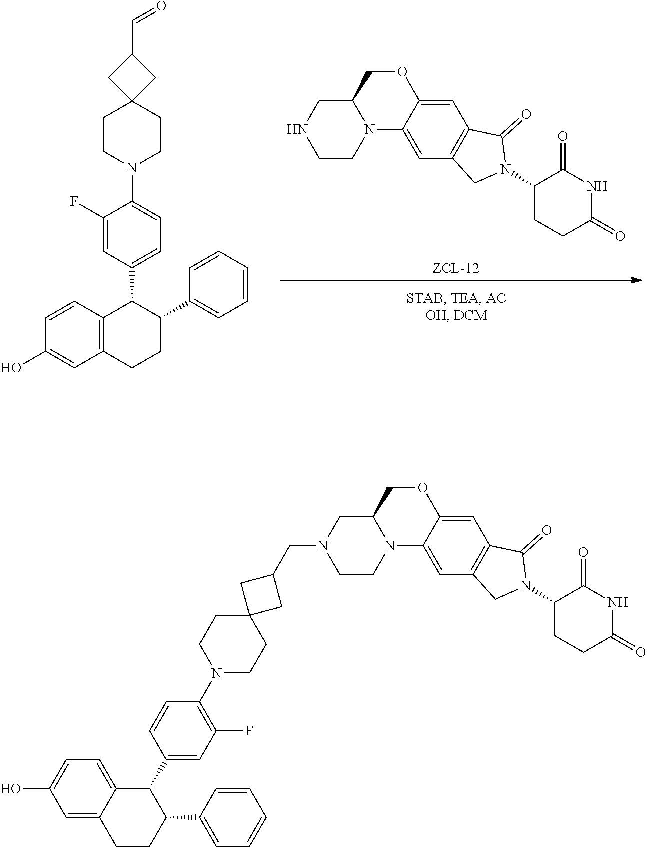 [SMILES: O=CC1CC2(CCN(c3ccc([C@H]4c5ccc(O)cc5CC[C@H]4c4ccccc4)cc3F)CC2)C1 ; O=C1CC[C@H](N2Cc3cc4c(cc3C2=O)OC[C@@H]2CNCCN42)C(=O)N1 ; "ZCL-12" ; O=C1CC[C@H](N2Cc3cc4c(cc3C2=O)OC[C@@H]2CN(CC3CC5(CCN(c6ccc([C@H]7c8ccc(O)cc8CC[C@H]7c7ccccc7)cc6F)CC5)C3)CCN42)C(=O)N1]

To a mixture of 7-(2-fluoro-4-((1S,2R)-6-hydroxy-2-phenyl-1,2,3,4-tetrahydronaphthalen-1-yl)phenyl)-7-azaspiro[3.5]nonane-2-carbaldehyde (26.5 mg, 0.057 mmol 1 eq.), (S)-3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione (22.13 mg, 0.062 mmol 1.1 eq.) TEA (8.6 mg, 0.085 mmol, 1.5 eq.) in DCM (2 mL) was added acetic acid (5.8 mg, 0.09 mmol, 1.7 eq.) followed by sodium triacetoxyborohydride (23.96 mg, 0.11 mmol 2.0 eq.) The resultant mixture was then stirred at 25° C. The reaction was cooled to 20° C. and concentrated under vacuum. The residue was purified by Prep-HPLC(acetonitrile/0.05% formic acid) to afford (S)-3-((S)-3-((7-(2-fluoro-4-((1S,2R)-6-hydroxy-2-phenyl-1,2,3,4-tetrahydronaphthalen-1-yl)phenyl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (28.81 mg, 59.4% yield) as a white solid. LC-MS (ESI, m/z): mass calcd. For C₅₀H₅₅N₅O₆, 821.42; found, 822.4 [M+H]⁺.

¹H NMR (400 MHZ, DMSO) § 10.93 (s, 1H), 9.24-9.09 (m, 1H), 7.15-7.00 (m, 4H), 6.93 (s, 1H), 6.85-6.76 (m, 2H), 6.64-6.57 (m, 2H), 6.56-6.41 (m, 3H), 6.31-6.21 (m, 1H), 5.07-4.98 (m, 1H), 4.44 (d, J=5.2 Hz, 1H), 4.32-4.09 (m, 3H), 3.92-3.75 (m, 2H), 3.18-3.08 (m, 2H), 3.07-2.81 (m, 10H), 2.79-2.63 (m, 1H), 2.63-2.56 (m, 1H), 2.43-2.27 (m, 3H), 2.25-2.03 (m, 2H), 2.02-1.86 (m, 3H), 1.76-1,55 (m, 4H), 1.52-1.34 (m, 4H),

### Compound A153: (S)-3-(1′-((2-(5-((1R,2R)-6-hydroxy-2-phenyl-1,2,3,4-tetrahydronaphthalen-1-yl)pyrimidin-2-yl)-2-azaspiro[3.5]nonan-7-yl)methyl)-6-oxo-6,8-dihydro-2H,7H-spiro[furo[2,3-e]isoindole-3,4′-piperidin]-7-yl)piperidine-2,6-dione

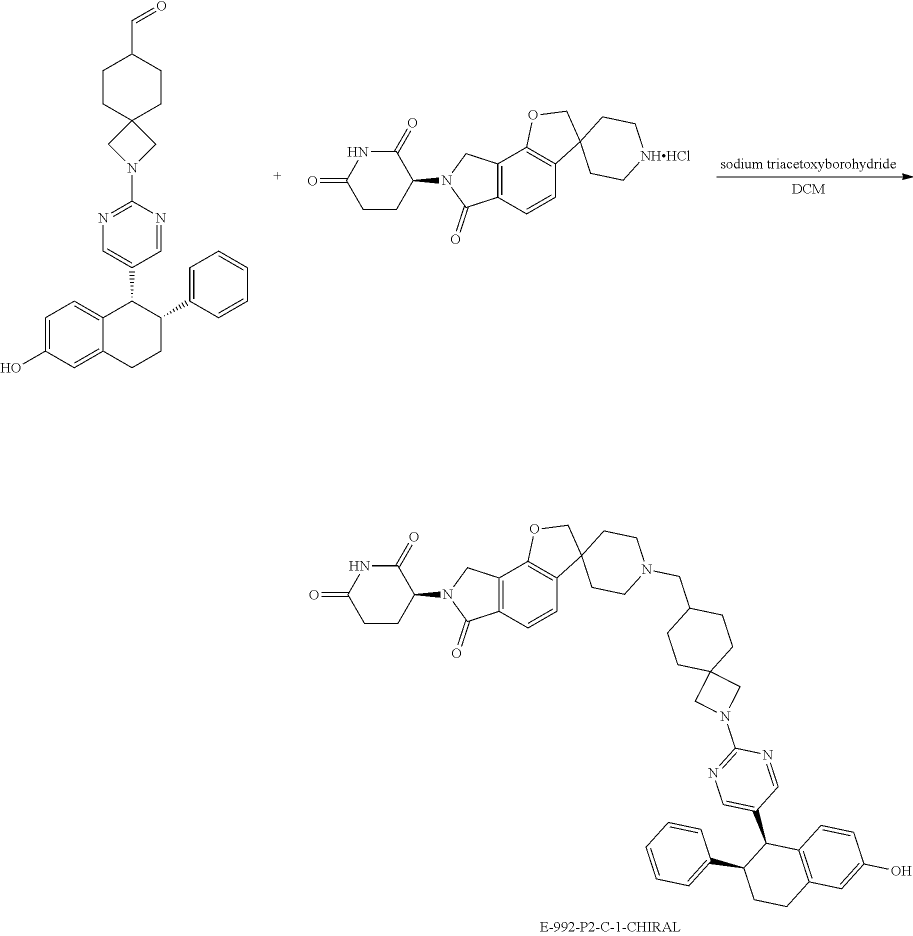

To a mixture of 2-(5-((1R,2R)-6-hydroxy-2-phenyl-1,2,3,4-tetrahydronaphthalen-1-yl)pyrimidin-2-yl)-2-azaspiro[3.5]nonane-7-carbaldehyde (30.0 mg, 66.1 μmol, 1 eq.), (S)-3-(6-oxo-6,8-dihydro-2H,7H-spiro[furo[2,3-e]isoindole-3,4′-piperidin]-7-yl)piperidine-2,6-dione hydrochloride (31,1 mg,, 79.4 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was by Prep-HPLC(acetonitrile/0.05% formic acid) to afford (S)-3-(1′-((2-(5-((1R,2R)-6-hydroxy-2-phenyl-1,2,3,4-tetrahydronaphthalen-1-yl)pyrimidin-2-yl)-2-azaspiro[3.5]nonan-7-yl)methyl)-6-oxo-6,8-dihydro-2H,7H-spiro[furo[2,3-e]isoindole-3,4′-piperidin]-7-yl)piperidine-2,6-dione (22.64 mg, 28.55 μmol, 43.2%) as a white solid. LC-MS purity: 99.8% (UV at 254 nm), 793.3 [M+H]⁺.

¹H NMR (400 MHZ, DMSO) δ 10.97 (s, 1H), 8.30 (s, 1H), 7.39 (d, J=7.6 Hz, 1H), 7.28-7.15 (m, 6H), 6.95 (d, J=7.2 Hz, 2H), 6.69 (d, J=8.4 Hz, 1H), 6.61 (s, 1H), 6.53-6.48 (m, 1H), 5.12-5.05 (m, 1H), 4.54-4.48 (m, 2H), 4.41-4.34 (m, 1H), 4.24-4.17 (m, 1H), 4.12 (d, J=4.8 Hz, 1H), 3.57 (s, 2H), 3.52 (s, 2H), 3.37-3.31 (m, 2H), 3.01-2.87 (m, 3H), 2.82-2,76 (m, 2H), 2.63-2.53 (m, 1H), 2.44-2.33 (m, 1H), 2.10 (d, J=6.8 Hz, 2H), 2.01-1.86 (m, 6H), 1.81 (d, J=12.0 Hz, 3H), 1.72-1.62 (m, 4H), 1.52-1.35 (m, 3H), 0.98-0.83 (m, 2H).

Compound A154: (S)-3-(1′-((2-(5-((1S,2S)-6-hydroxy-2-phenyl-1,2,3,4-tetrahydronaphthalen-1-yl)pyrimidin-2-yl)-2-azaspiro[3.5]nonan-7-yl)methyl)-6-oxo-6,8-dihydro-2H,7H-spiro[furo[2,3-e]isoindole-3,4′-piperidine-2,6-dione

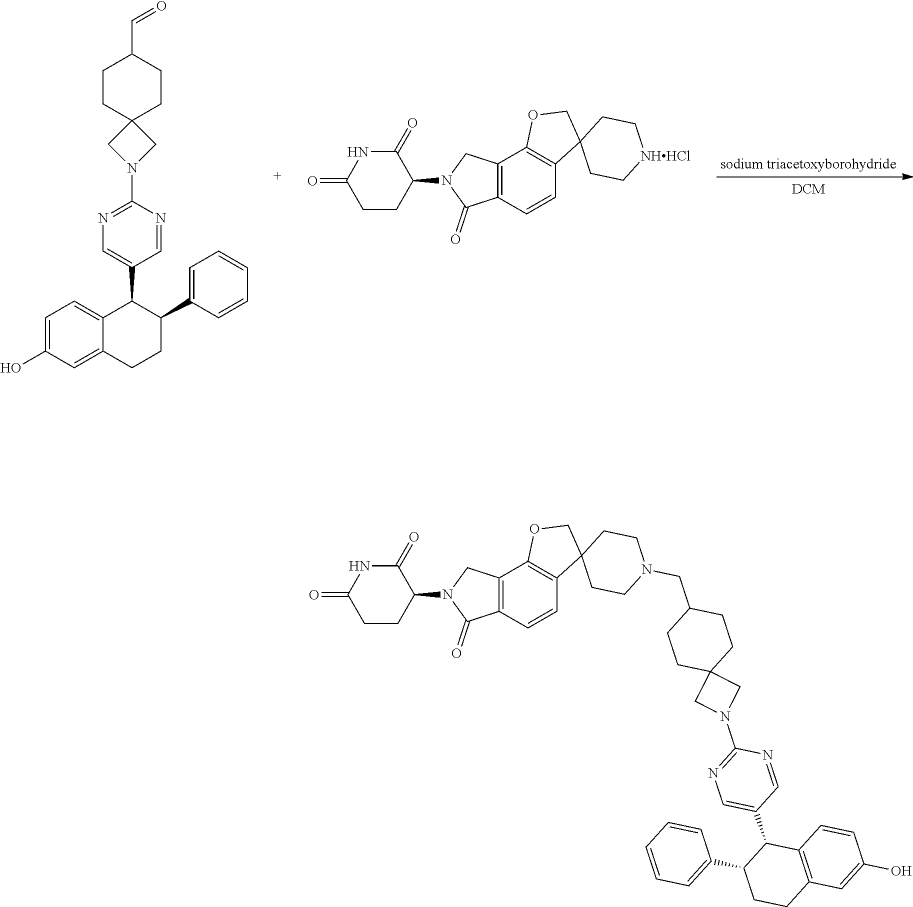

To a mixture of 2-(5-((1S,2S)-6-hydroxy-2-phenyl-1,2,3,4-tetrahydronaphthalen-1-yl)pyrimidin-2-yl)-2-azaspiro[3.5]nonane-7-carbaldehyde (30.0 mg, 66.1 μmol, 1 eq.), (S)-3-(6-oxo-6,8-dihydro-2H,7H-spiro[furo[2,3-e]isoindole-3,4′-piperidin]-7-yl)piperidine-2,6-dione hydrochloride (31,1 mg,, 79.4 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was by Prep-HPLC(acetonitrile/0.05% formic acid) to afford (S)-3-(1′-((2-(5-((1S,2S)-6-hydroxy-2-phenyl-1,2,3,4-tetrahydronaphthalen-1-yl)pyrimidin-2-yl)-2-azaspiro[3.5]nonan-7-yl)methyl)-6-oxo-6,8-dihydro-2H,7H-spiro[furo[2,3-e]isoindole-3,4′-piperidin]-7-yl)piperidine-2,6-dione (22.64 mg, 28.55 μmol, 43.2%) as a white solid. LC-MS purity: 99.8% (UV at 254 nm), 793.3 [M+H]⁺, ¹H NMR (400 MHZ, DMSO) δ 10.97 (s, 1H), 8.26 (s, 1H), 7.44-7.36 (m, 1H), 7.26-7.15 (m, 6H), 6.95 (d, J=7.2 Hz, 2H), 6.69 (d, J=8.4 Hz, 1H), 6.61 (d, J=2.0 Hz, 1H), 6.54-6.48 (m, 1H), 5.13-5.04 (m, 1H), 4.50 (t, J=10.2 Hz, 2H), 4.40-4.34 (m, 1H), 4.24-4.17 (m, 1H), 4.12 (d, J=4.8 Hz, 1H), 3.57 (s, 2H), 3.52 (s, 2H), 3.36-3,29 (m, 2H), 3.00-2.86 (m, 3H), 2.82-2,75 (m, 2H), 2.66-2.54 (m, 1H), 2.47-2.34 (m, 1H), 2.10 (d, J=6.8 Hz, 2H), 2.00-1.86 (m, 6H), 1.84-1.75 (m, 3H), 1.72-1.61 (m, 4H), 1.51-1.36 (m, 3H), 0.95-0.82 (m, 2H),

### Compound A155: (S)-3-((R)-7-((2-(5-((1R,2R)-6-hydroxy-2-phenyl-1,2,3,4-tetrahydronaphthalen-1-yl)pyrimidin-2-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

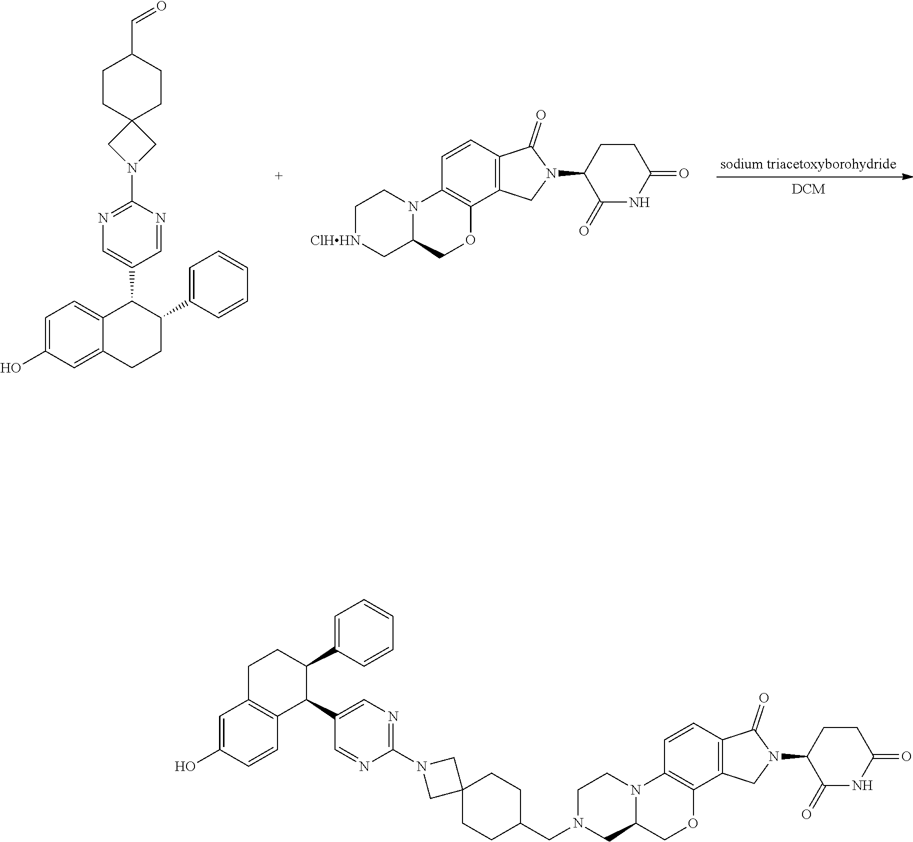

To a mixture of 2-(5-((1R,2R)-6-hydroxy-2-phenyl-1,2,3,4-tetrahydronaphthalen-1-yl)pyrimidin-2-yl)-2-azaspiro[3.5]nonane-7-carbaldehyde (30.0 mg, 66.1 μmol, 1 eq.), (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione hydrochloride (31,1 mg,, 79.4 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was by Prep-HPLC(acetonitrile/0.05% formic acid) to afford (S)-3-((R)-7-((2-(5-((1R,2R)-6-hydroxy-2-phenyl-1,2,3,4-tetrahydronaphthalen-1-yl)pyrimidin-2-yl)-2-azaspiro[3.5]nonan-7-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (25.32 mg, 31.89 μmol, 48.2%) as a white solid. LC-MS purity: 92.2% (UV at 254 nm), 794,4 [M+H]⁺.

¹H NMR (400 MHZ, DMSO) δ 10.93 (s, 1H), 7.28-7.15 (m, 6H), 7.02-6.91 (m, 3H), 6.68 (d, J=8.4 Hz, 1H), 6.61 (d, J=2.0 Hz, 1H), 6.53-6.46 (m, 1H), 5.08-4.96 (m, 1H), 4.39-4.33 (m, 1H), 4.29-4,21 (m, 1H), 4.15-4.06 (m, 2H), 3.99-3.91 (m, 1H), 3.81 (d, J=10.8 Hz, 1H), 3.56-3.51 (m, 3H), 3.36-3.30 (m, 1H), 3.19-3.14 (m, 1H), 3.00-2.84 (m, 5H), 2.76-2.59 (m, 2H), 2.59-2.53 (m, 1H), 2.43-2.34 (m, 1H), 2.17-2.02 (m, 3H), 1.99-1.91 (m, 2H), 1.83-1.76 (m, 3H), 1.73-1.62 (m, 3H), 1.53-1.36 (m, 3H), 1.01 (t, J=7.2 Hz, 1H), 0.97-0.82 (m, 2H),

### Compound A156: (S)-3-((R)-7-((2-(5-((1S,2S)-6-hydroxy-2-phenyl-1,2,3,4-tetrahydronaphthalen-1-yl)pyrimidin-2-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

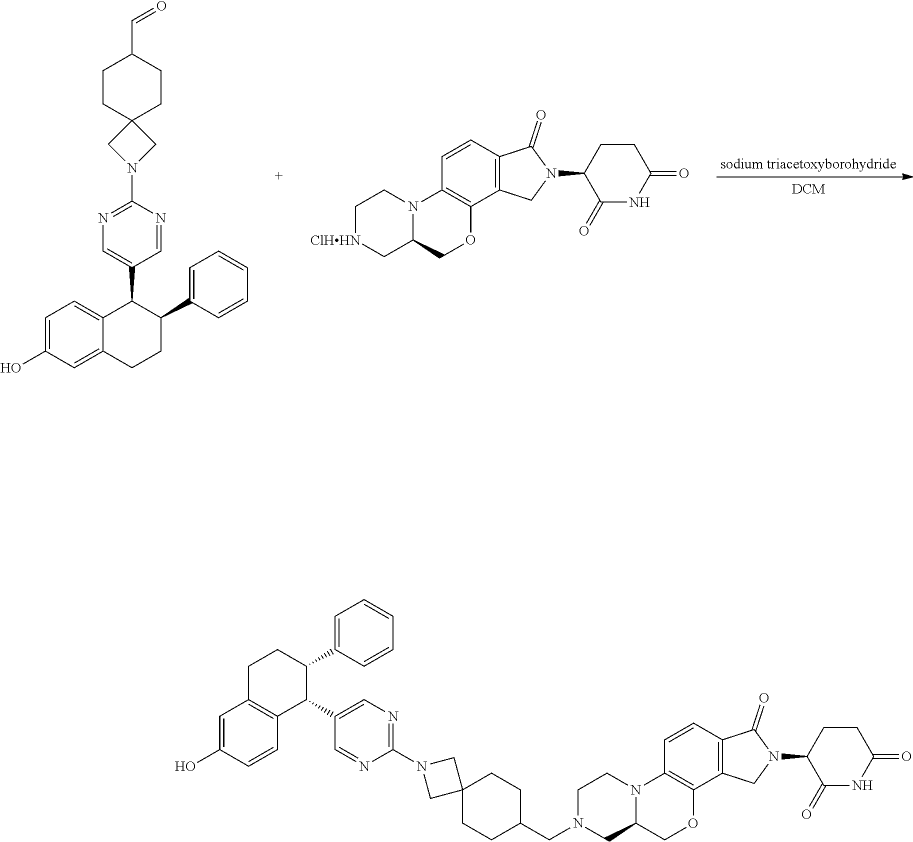

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (30.0 mg, 66.1 μmol, 1 eq.), (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione hydrochloride (31,1 mg., 79.4 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was by Prep-HPLC(acetonitrile/0.05% formic acid) to afford (S)-3-((R)-7-((2-(5-((1S,2S)-6-hydroxy-2-phenyl-1,2,3,4-tetrahydronaphthalen-1-yl)pyrimidin-2-yl)-2-azaspiro[3.5]nonan-7-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (25.32 mg, 31.89 μmol, 48.2%) as a white solid. LC-MS purity: 92.2% (UV at 254 nm), 794.3 [M+H]⁺.

¹H NMR (400 MHZ, DMSO) δ 11.00 (s, 1H), 7.31-7.22 (m, 6H), 7.07-6.94 (m, 3H), 6.74 (d, J=8.4 Hz, 1H), 6.67 (d, J=2.0 Hz, 1H), 6.59-6.53 (m, 1H), 5.13-5.03 (m, 1H), 4.45-4.37 (m, 1H), 4.34-4,27 (m, 1H), 4.22-4.11 (m, 2H), 4.05-3.98 (m, 1H), 3.89-3.84 (m, 1H), 3.63-3.60 (m, 2H), 3.58 (s, 3H), 3.24-3.19 (m, 1H), 3.13-2.86 (m, 6H), 2.79 (t, J=10.4 Hz, 1H), 2.68-2.58 (m, 1H), 2.51-2.38 (m, 1H), 2.24-2.08 (m, 3H), 2.05-1.95 (m, 2H), 1.89-1.81 (m, 3H), 1.79-1.69 (m, 3H), 1.58-1.42 (m, 3H), 1.03-0.87 (m, 2H),

### Compound A157: (S)-3-((S)-3-((2-(5-((1R,2R)-6-hydroxy-2-phenyl-1,2,3,4-tetrahydronaphthalen-1-yl)pyrimidin-2-yl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

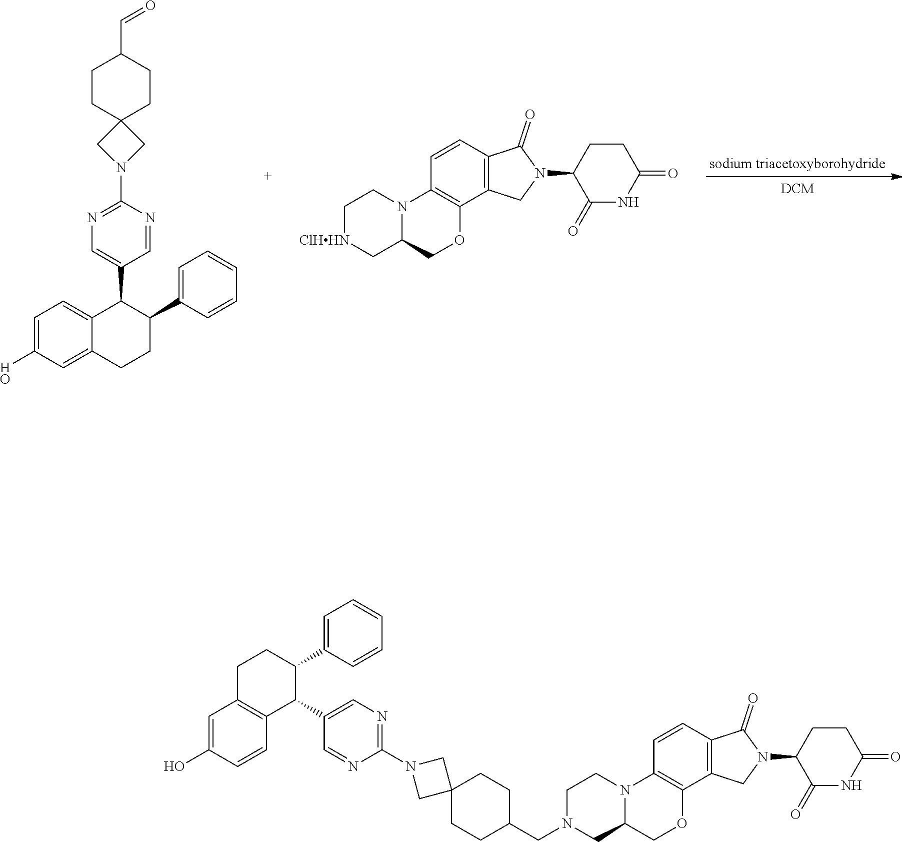

To a mixture of 2-(5-((1R,2R)-6-hydroxy-2-phenyl-1,2,3,4-tetrahydronaphthalen-1-yl)pyrimidin-2-yl)-2-azaspiro[3.5]nonane-7-carbaldehyde (30.0 mg, 66.1 μmol, 1 eq.), (S)-3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione hydrochloride (31,1 mg,, 79.4 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was by Prep-HPLC(acetonitrile/0.05% formic acid) to afford (S)-3-((S)-3-((2-(5-((1R,2R)-6-hydroxy-2-phenyl-1,2,3,4-tetrahydronaphthalen-1-yl)pyrimidin-2-yl)-2-azaspiro[3.5]nonan-7-yl)methyl)-8-oxo-1,2,3,4,4a,5,8,10-octahydro-9H-pyrazino[11,2′: 4,5][1,4]oxazino[2,3-f]isoindol-9-yl)piperidine-2,6-dione (20.53 mg, 25.86 μmol, 39.1%) as a white solid. LC-MS purity: 92.2% (UV at 254 nm), 794,4 [M+H]⁺.

¹H NMR (400 MHZ, DMSO) δ 10.94 (s, 1H), 7.26-7.15 (m, 5H), 7.03 (s, 1H), 6.94 (d, J=6.6 Hz, 3H), 6.68 (d, J=8.4 Hz, 1H), 6.61 (s, 1H), 6.55-6.47 (m, 1H), 5.06-4.97 (m, 1H), 4.32-4,20 (m, 2H), 4.17-4.07 (m, 2H), 3.97-3.82 (m, 2H), 3.81-3.72 (m, 2H), 3.37-3.30 (m, 2H), 3.19-3.01 (m, 2H), 3.02-2.80 (m, 6H), 2.75 (t, J=10.4 Hz, 1H), 2.61-2.54 (m, 1H), 2.41-2.28 (m, 1H), 2.16-2.03 (m, 3H), 2.00-1.90 (m, 2H), 1.84-1.74 (m, 3H), 1.72-1.61 (m, 3H), 1.53-1.36 (m, 3H), 0.98-0.84 (m, 2H),

### Compound A158: (S)-3-((S)-3-((2-(5-((1S,2S)-6-hydroxy-2-phenyl-1,2,3,4-tetrahydronaphthalen-1-yl)pyrimidin-2-yl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

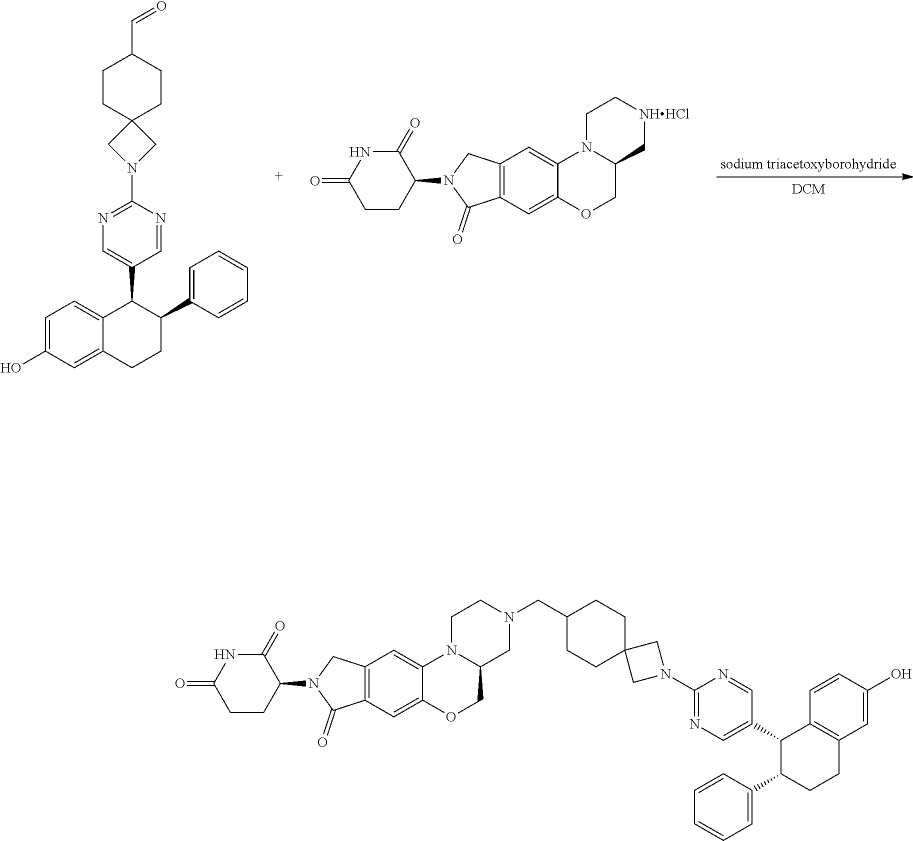

To a mixture of 2-(5-((1S,2S)-6-hydroxy-2-phenyl-1,2,3,4-tetrahydronaphthalen-1-yl)pyrimidin-2-yl)-2-azaspiro[3.5]nonane-7-carbaldehyde (30.0 mg, 66.1 μmol, 1 eq.) (S)-3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione hydrochloride (31,1 mg,, 79.4 μmol, 1,2 eq.) and TEA (9.6 mg, 0.095 mmol, 1.5 eq.) in DCM (2.00 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was by Prep-HPLC(Acetonitrile/0.05% Formate acid) to afford (S)-3-((S)-3-((2-(5-((1S,2S)-6-hydroxy-2-phenyl-1,2,3,4-tetrahydronaphthalen-1-yl)pyrimidin-2-yl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (17.82 mg, 22.44 μmol, 33.9%) as a white solid. LC-MS purity: 99.8% (UV at 254 nm), 794.3 [M+H]⁺.

¹H NMR (400 MHZ, DMSO) δ 10.93 (s, 1H), 8.31 (s, 1H), 7.25-7.12 (m, 5H), 7.03 (s, 1H), 6.97-6.88 (m, 3H), 6.68 (d, J=8.4 Hz, 1H), 6.61 (s, 1H), 6.53-6.44 (m, 1H), 5.06-4.98 (m, 1H), 4.33-4.19 (m, 2H), 4.18-4.08 (m, 2H), 3.92-3.75 (m, 3H), 3.58-3.55 (m, 2H), 3.36-3.32 (m, 1H), 3.17-3.12 (m, 1H), 3.00-2.85 (m, 5H), 2.79-2,70 (m, 1H), 2.66-2.51 (m, 2H), 2.40-2.30 (m, 1H), 2.19-2.02 (m, 3H), 1.99-1.89 (m, 2H), 1.84-1.75 (m, 3H), 1.74-1.63 (m, 3H), 1.53-1.36 (m, 3H), 1.06-0.97 (m, 1H), 0.95-0.83 (m, 2H),

### Compound A163: (S)-3-((R)-7-((2-(2-fluoro-4-((1S,2S)-6-hydroxy-2-phenyl-1,2,3,4-tetrahydronaphthalen-1-yl)-5-methoxyphenyl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

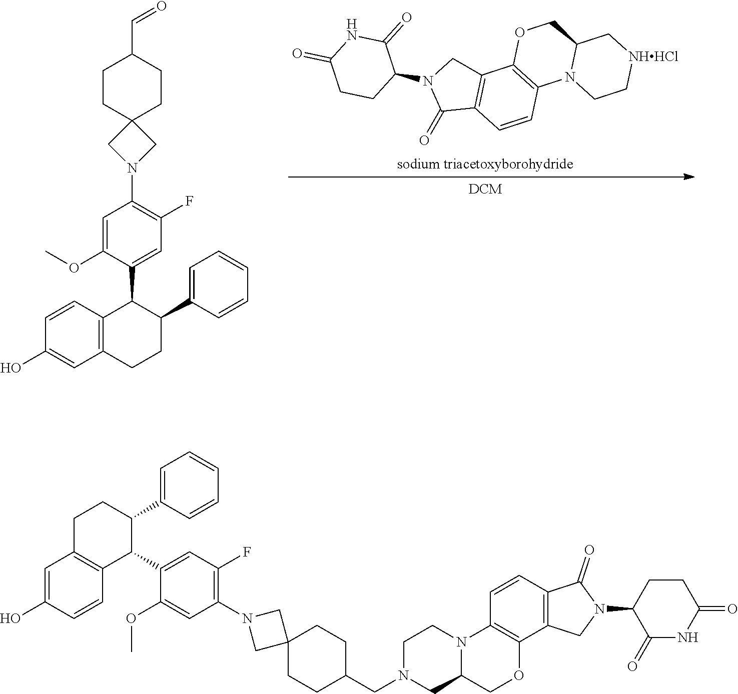

To a mixture of 2-(2-fluoro-4-((1S,2S)-6-hydroxy-2-phenyl-1,2,3,4-tetrahydronaphthalen-1-yl)-5-methoxyphenyl)-2-azaspiro[3.5]nonane-7-carbaldehyde (0.03 g, 1.0 eq, 0.06 mmol), (S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione (0.02 g, 1,1 eq, 0.07 mmol) in DCM (5.00 mL) was added sodium triacetoxyborohydride (0.03 g, 0.02 mL, 2.0 eq, 0.1 mmol). The resultant mixture was then stirred at 25° C. for 1 h. The residue was purified by Prep-HPLC(acetonitrile/0.05% formic acid) to afford (S)-3-((R)-7-((2-(2-fluoro-4-((1S,2S)-6-hydroxy-2-phenyl-1,2,3,4-tetrahydronaphthalen-1-yl)-5-methoxyphenyl)-2-azaspiro[3.5]nonan-7-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (28.88 mg, 60% yield) as a white solid. LC-MS purity: 99.7% (UV at 254 nm), 840.4 [M+H]⁺.

¹H NMR (400 MHZ, DMSO) δ 10.92 (s, 1H), 9.11 (s, 1H), 7.22-6.95 (m, 5H), 6.78 (d, J=6.8 Hz, 2H), 6.65-6.39 (m, 3H), 6.10 (d, J=13.6 Hz, 1H), 5,67 (d, J=8.0 Hz, 1H), 5.03 (dd, J=13.2, 5.2 Hz, 1H), 4.62 (d, J=5.2 Hz, 1H), 4.40-4.31 (m, 1H), 4.18 (dd, J=62.8, 16.8 Hz, 2H), 4.01-3.89 (m, 1H), 3.81 (d, J=11.6 Hz, 1H), 3.57-3.45 (m, 4H), 3.23-3.14 (m, 2H), 3.02-2.82 (m, 8H), 2.74 (d, J=10.4 Hz, 1H), 2.62-2.54 (m, 1H), 2.43-2.31 (m, 1H), 2.20-2.01 (m, 4H), 1.99-1.84 (m, 3H), 1.76-1.58 (m, 4H), 1.55-1.37 (m, 3H), 1.00-0.82 (m, 2H),

### Compound A164: (S)-3-((S)-3-((2-(2-fluoro-4-((1S,2S)-6-hydroxy-2-phenyl-1,2,3,4-tetrahydronaphthalen-1-yl)-5-methoxyphenyl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

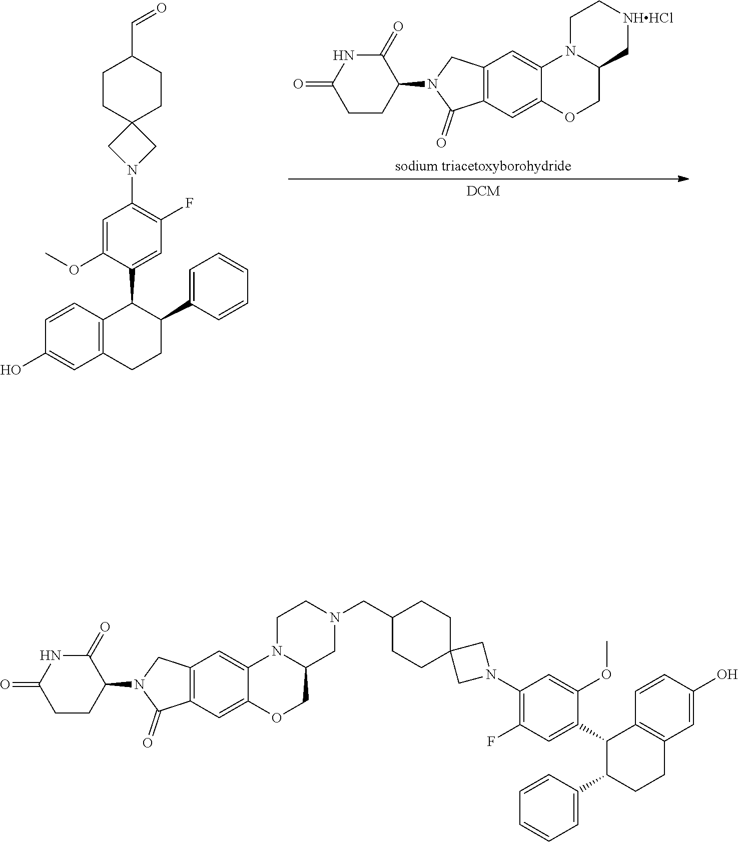

To a mixture of 2-(2-fluoro-4-((1S,2S)-6-hydroxy-2-phenyl-1,2,3,4-tetrahydronaphthalen-1-yl)-5-methoxyphenyl)-2-azaspiro[3.5]nonane-7-carbaldehyde (0.03 g, 1 eq, 0.06 mmol), (S)-3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione (0.02 g, 1,1 eq, 0.07 mmol) in DCM (5.00 mL) was added sodium triacetoxyborohydride (0.03 g, 0.02 mL, 2.0 eq, 0.1 mmol). The resultant mixture was then stirred at 25° C. for 1 h. The residue was purified by Prep-HPLC(acetonitrile/0.05% formic acid) to afford (S)-3-((S)-3-((2-(2-fluoro-4-((1S,2S)-6-hydroxy-2-phenyl-1,2,3,4-tetrahydronaphthalen-1-yl)-5-methoxyphenyl)-2-azaspiro[3.5]nonan-7-yl)methyl)-8-oxo-1,2,3,4,4a,5,8,10-octahydro-9H-pyrazino[11,2′: 4,5][1,4]oxazino[2,3-f]isoindol-9-yl)piperidine-2,6-dione (21.04 mg, 40% yield) as a white solid. LC-MS purity: 99.7% (UV at 254 nm), 840.4 [M+H]⁺.

¹H NMR (400 MHz, DMSO) δ 10.92 (s, 1H), 9.11 (s, 1H), 7.14-7.00 (m, 4H), 6.93 (s, 1H), 6.78 (d, J=6.8 Hz, 2H), 6.60-6.43 (m, 3H), 6.10 (d, J=13.6 Hz, 1H), 5,67 (d, J=8.0 Hz, 1H), 5.02 (dd, J=13.2, 4.8 Hz, 1H), 4.62 (d, J=5.2 Hz, 1H), 4.32-4.11 (m, 3H), 3.93-3.76 (m, 2H), 3.57-3.45 (m, 4H), 3.24-3.14 (m, 2H), 2.98-2.84 (m, 8H), 2.80-2,72 (m, 1H), 2.64-2.57 (m, 1H), 2.39-2.30 (m, 1H), 2.17-2.07 (m, 3H), 1.98-1.84 (m, 3H), 1.75-1.59 (m, 4H), 1.54-1.38 (m, 3H), 1.24 (s, 1H), 0.98-0.85 (m, 2H),

### Compound A165: (S)-3-((R)-7-((2-(2-fluoro-4-((1R,2R)-6-hydroxy-2-phenyl-1,2,3,4-tetrahydronaphthalen-1-yl)-5-methoxyphenyl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

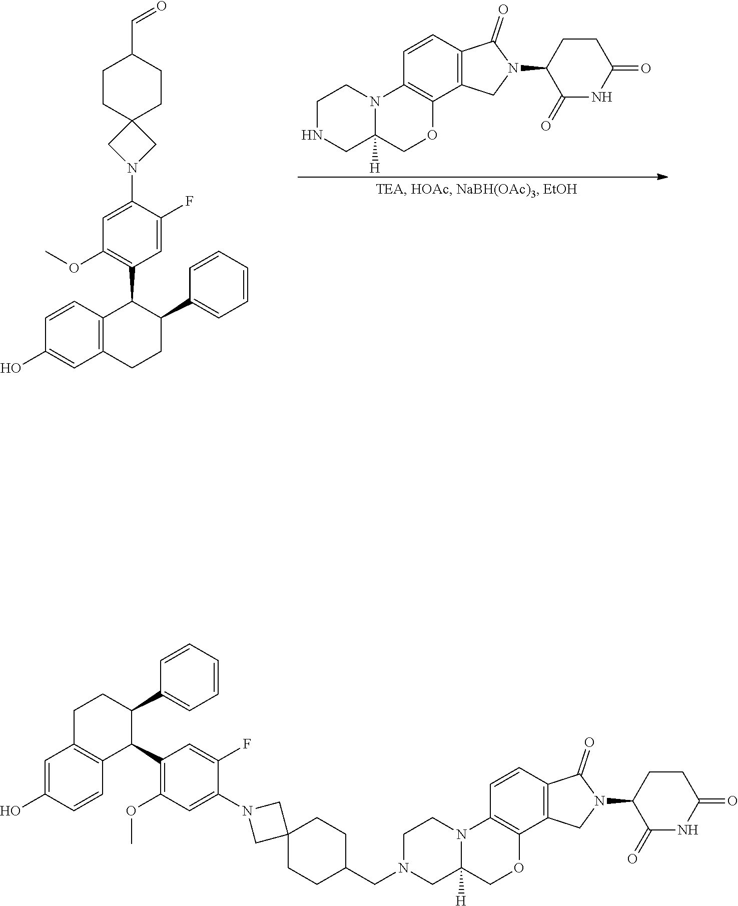 [SMILES: COc1cc(N2CC3(CCC(C=O)CC3)C2)c(F)cc1[C@@H]1c2ccc(O)cc2CC[C@@H]1c1ccccc1 ; O=C1CC[C@H](N2Cc3c(ccc4c3OC[C@H]3CNCCN43)C2=O)C(=O)N1 ; COc1cc(N2CC3(CCC(CN4CCN5c6ccc7c(c6OC[C@H]5C4)CN([C@H]4CCC(=O)NC4=O)C7=O)CC3)C2)c(F)cc1[C@H]1c2ccc(O)cc2CC[C@H]1c1ccccc1]

To a mixture of 72-(5-((1S,2S)-6-hydroxy-2-phenyl-1,2,3,4-tetrahydronaphthalen-1-yl)pyridin-2-yl)-2-azaspiro[3.5]nonane-7-carbaldehyde (30 mg, 0.063 mmol, 1 eq.), (S)-2-(2,6-dioxopiperidin-3-yl)-5-(piperidin-4-yl)-3,5-dihydro-1H-pyrrolo[3,4-c]pyridine-1,4 (2H)-dione (30 mg, 0.07 mmol, 1,2 eq.), TEA (9.6 mg, 0.095 mmol, 1.5 eq.) in DCM (2.0 mL) was added acetic acid (6.4 mg, 0.11 mmol, 1.7 eq.) followed by sodium triacetoxyborohydride (27 mg, 0.13 mmol, 2 eq.). The mixture was stirred at room temperature for 30 minutes and concentrated. The residue was purified by reverse-phase chromatography (acetonitrile/0.05% formic acid) to afford (S)-3-((R)-7-((2-(2-fluoro-4-((1R,2R)-6-hydroxy-2-phenyl-1,2,3,4-tetrahydronaphthalen-1-yl)-5-methoxyphenyl)-2-azaspiro[3.5]nonan-7-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (12.78 mg, 39.7% yield) as white solid. LC-MS purity: 99.8% (UV at 254 nm), 840.4 [M+H]⁺.

¹H NMR (400 MHZ, DMSO) δ 10.92 (s, 1H), 7.19-6.97 (m, 5H), 6.78 (d, J=6.8 Hz, 2H), 6.64-6.46 (m, 3H), 6.10 (d, J=13.6 Hz, 1H), 5,67 (d, J=8.0 Hz, 1H), 5.07-4.93 (m, 1H), 4.62 (d, J=5.2 Hz, 1H), 4.40-4.34 (m, 1H), 4.31-4.09 (m, 2H), 3.97 (d, J=8.8 Hz, 1H), 3.86-3.79 (m, 1H), 3.62-3.46 (m, 5H), 3.29-3.07 (m, 5H), 2.91 (d, J=10.6 Hz, 6H), 2.59 (s, 1H), 2.42-2.34 (m, 1H), 2.17-1.88 (m, 6H), 1.72-1.36 (m, 7H), 1.24 (s, 1H), 0.98-0.84 (m, 2H),

### Compound A166: (S)-3-(1′-((2-(2-fluoro-4-((1S,2S)-6-hydroxy-2-phenyl-1,2,3,4-tetrahydronaphthalen-1-yl)-5-methoxyphenyl)-2-azaspiro[3.5]nonan-7-yl)methyl)-7-oxo-5,7-dihydro-2H,6H-spiro[furo[2,3-f]isoindole-3,4′-piperidin]-6-yl)piperidine-2,6-dione

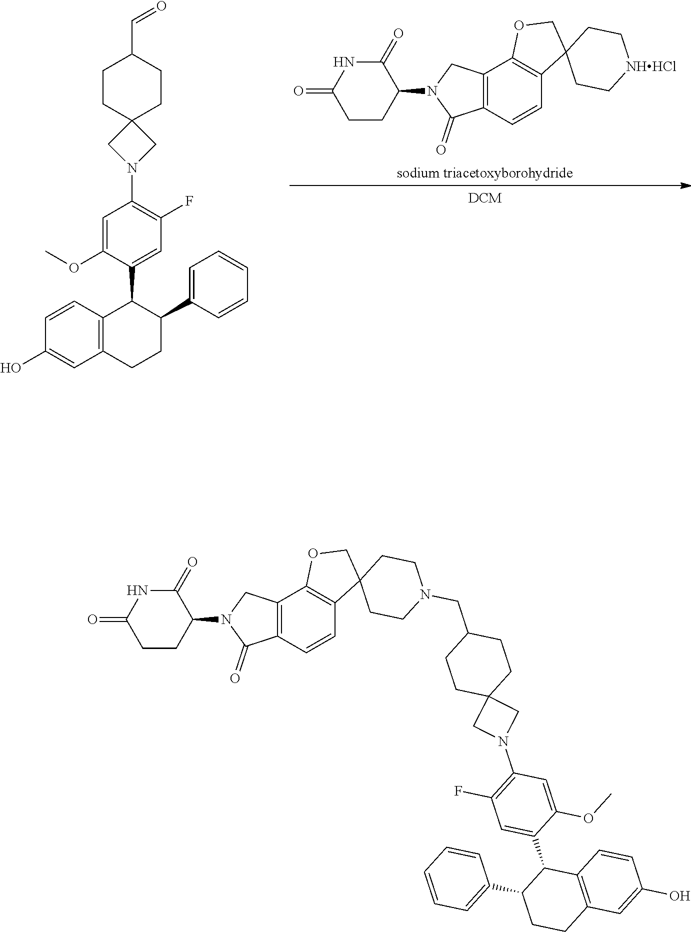

To a mixture of 2-(2-fluoro-4-((1S,2S)-6-hydroxy-2-phenyl-1,2,3,4-tetrahydronaphthalen-1-yl)-5-methoxyphenyl)-2-azaspiro[3.5]nonane-7-carbaldehyde (0.03 g, 1 eq, 0.06 mmol), (S)-3-(6-oxo-6,8-dihydro-2H,7H-spiro[furo[2,3-e]isoindole-3,4′-piperidin]-7-yl)piperidine-2,6-dione hydrochloride (0.02 g, 1,1 eq, 0.07 mmol) in DCM (5.00 mL) was added sodium triacetoxyborohydride (0.03 g, 0.02 mL, 2.0 eq, 0.1 mmol). The resultant mixture was then stirred at 25° C. for 1 h. The residue was purified by Prep-HPLC(acetonitrile/0.05% formic acid) to afford (S)-3-(1′-((2-(2-fluoro-4-((1S,2S)-6-hydroxy-2-phenyl-1,2,3,4-tetrahydronaphthalen-1-yl)-5-methoxyphenyl)-2-azaspiro[3.5]nonan-7-yl)methyl)-6-oxo-6,8-dihydro-2H,7H-spiro[furo[2,3-e]isoindole-3,4′-piperidin]-7-yl)piperidine-2,6-dione (25.5 mg, 50% yield) as a white solid. LC-MS purity: 99.4% (UV at 254 nm), 839.4 [M+H]⁺.

¹H NMR (400 MHZ, DMSO-d₆) δ 10.96 (s, 1H), 8.26 (s, 1H), 7.45 (s, 1H), 7.12-7.02 (m, 3H), 7.00 (s, 1H), 6.78 (d, J=6.8 Hz, 2H), 6.58-6.54 (m, 2H), 6.49-6.46 (m, 1H), 6.10 (d, J=13.6 Hz, 1H), 5,67 (d, J-8.4 Hz, 1H), 5.09-5.05 (m, 1H), 4.63-4.62 (m, 1H), 4.55 (s, 2H), 4.34 (d, J=16.8 Hz, 1H), 4.21 (d, J=17.2 Hz, 1H), 3.57-3.46 (m, 4H), 3.24-3,20 (m, 2H), 2.93-2,78 (m, 8H), 2.63-2.57 (m, 1H), 2.43-2.31 (m, 1H), 2.18-2.09 (m, 2H), 2.00-1.86 (m, 7H), 1.71-1.60 (m, 5H), 1.52-1.40 (m, 3H), 0.97-0.85 (m, 2H) . . .

### Compound A168: (S)-3-(1′-((2-(2-fluoro-4-((1R,2R)-6-hydroxy-2-phenyl-1,2,3,4-tetrahydronaphthalen-1-yl)-5-methoxyphenyl)-2-azaspiro[3.5]nonan-7-yl)methyl)-6-oxo-6,8-dihydro-2H,7H-spiro[furo[2,3-e]isoindole-3,4′-piperidin]-7-yl)piperidine-2,6-dione

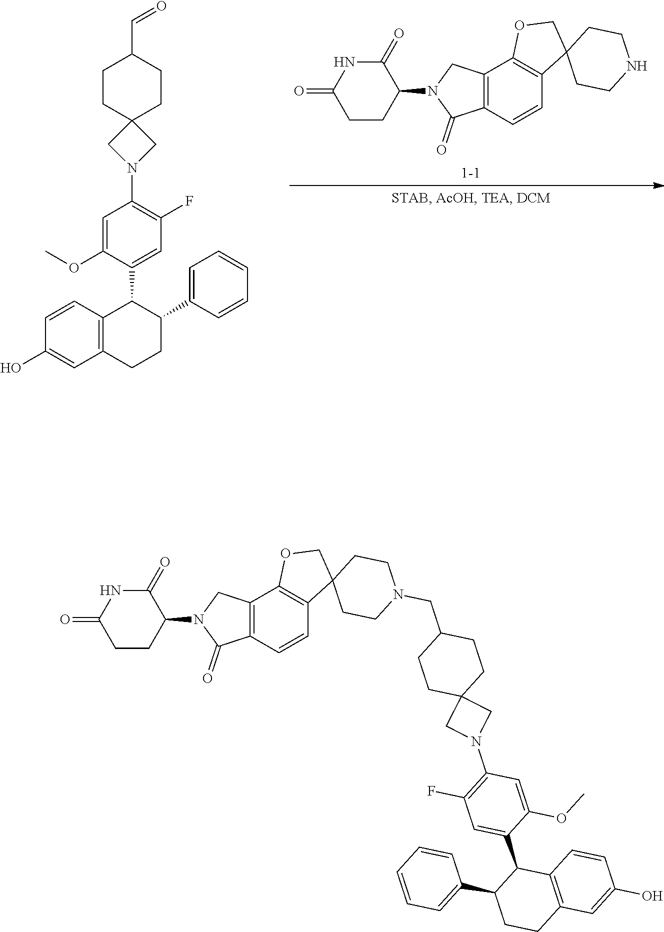 [SMILES: COc1cc(N2CC3(CCC(C=O)CC3)C2)c(F)cc1[C@H]1c2ccc(O)cc2CC[C@H]1c1ccccc1 ; O=C1CC[C@H](N2Cc3c(ccc4c3OCC43CCNCC3)C2=O)C(=O)N1 ; COc1cc(N2CC3(CCC(CN4CCC5(CC4)COc4c5ccc5c4CN([C@H]4CCC(=O)NC4=O)C5=O)CC3)C2)c(F)cc1[C@H]1c2ccc(O)cc2CC[C@H]1c1ccccc1]

To a mixture of 2-(2-fluoro-4-((1R,2R)-6-hydroxy-2-phenyl-1,2,3,4-tetrahydronaphthalen-1-yl)-5-methoxyphenyl)-2-azaspiro[3.5]nonane-7-carbaldehyde (35.0 mg, 1 Eq, 70.1 μmol), (S)-3-(6-oxo-6,8-dihydro-2H,7H-spiro[furo[2,3-e]isoindole-3,4′-piperidin]-7-yl)piperidine-2,6-dione (29.9 mg, 1,2 Eq, 84.1 μmol), TEA (10.6 mg, 0.13 mmol, 1.5 eq.) in DCM (4.0 mL) was added acetic acid (7.6 mg, 0.11 mmol, 1.8 eq.) followed by sodium triacetoxyborohydride (29.7 mg, 20.8 L, 2 Eq, 140 μmol). The mixture was stirred at room temperature for 50 minutes and concentrated. The residue was purified by reverse-phase chromatography (acetonitrile/0.05% formic acid) to afford (S)-3-(1′-((2-(2-fluoro-4-((1R,2R)-6-hydroxy-2-phenyl-1,2,3,4-tetrahydronaphthalen-1-yl)-5-methoxyphenyl)-2-azaspiro[3.5]nonan-7-yl)methyl)-6-oxo-6,8-dihydro-2H,7H-spiro[furo[2,3-e]isoindole-3,4′-piperidin]-7-yl)piperidine-2,6-dione (25.84 mg, 30.80 μmol, 44.0%) as white solid.

LC-MS purity: 100% (UV at 254 nm), 839.5 [M+H]⁺.

¹H NMR (400 MHZ, DMSO) δ 10.96 (s, 1H), 9.56-8.58 (m, 1H), 7.39 (d, J=7.6 Hz, 1H), 7.26 (d, J=7.6 Hz, 1H), 7.13-7.05 (m, 3H), 6.78 (d, J=7.2 Hz, 2H), 6.59-6.53 (m, 2H), 6.50-6.45 (m, 1H), 6.10 (d, J=13.2 Hz, 1H), 5,68 (d, J=8.0 Hz, 1H), 5.08 (dd, J=13.2, 4.8 Hz, 1H), 4.62 (d, J=4.8 Hz, 1H), 4.51 (s, 2H), 4.38 (d, J=17.2 Hz, 1H), 4.21 (d, J=17.2 Hz, 1H), 3.22 (d, J=14.4 Hz, 2H), 2.93 (s, 3H), 2.84-2,78 (m, 2H), 2.69-2.55 (m, 2H), 2.49-2.34 (m, 2H), 2.25-2.04 (m, 4H), 2.00-1.84 (m, 8H), 1.76-1.58 (m, 6H), 1.56-1.33 (m, 4H), 1.25-1.15 (m, 1H), 0.95-0.85 (m, 2H),

### Compound A169: (S)-3-((S)-3-((2-(2-fluoro-4-((1R,2R)-6-hydroxy-2-phenyl-1,2,3,4-tetrahydronaphthalen-1-yl)-5-methoxyphenyl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

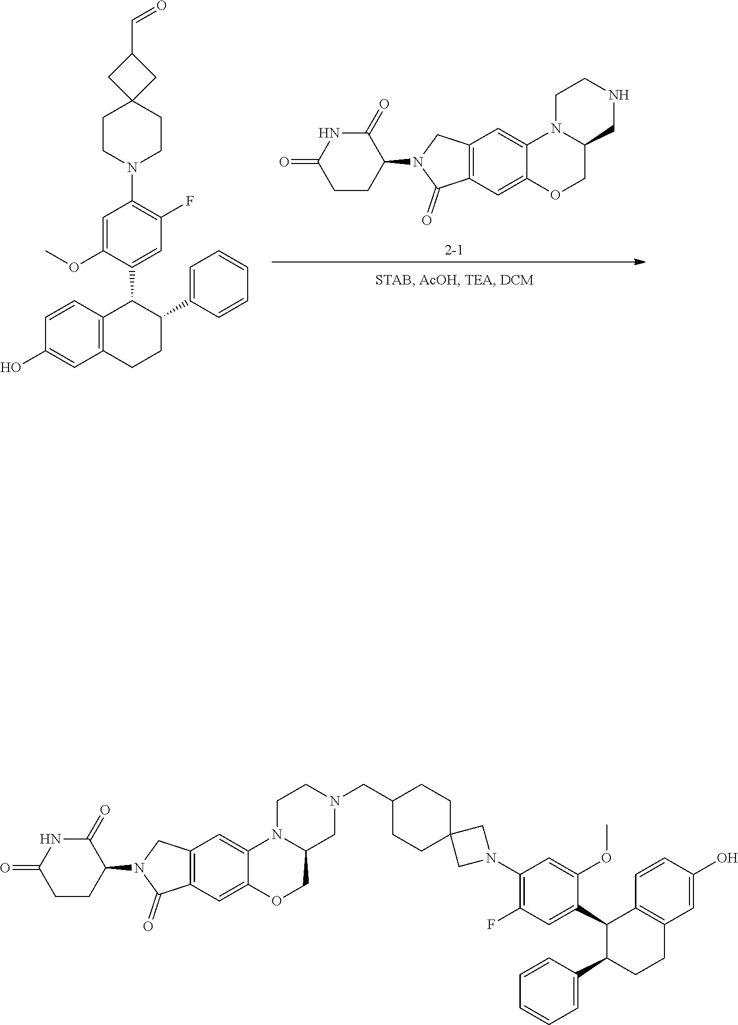 [SMILES: COc1cc(N2CCC3(CC2)CC(C=O)C3)c(F)cc1[C@H]1c2ccc(O)cc2CC[C@H]1c1ccccc1 ; O=C1CC[C@H](N2Cc3cc4c(cc3C2=O)OC[C@@H]2CNCCN42)C(=O)N1 ; COc1cc(N2CC3(CCC(CN4CCN5c6cc7c(cc6OC[C@@H]5C4)C(=O)N([C@H]4CCC(=O)NC4=O)C7)CC3)C2)c(F)cc1[C@H]1c2ccc(O)cc2CC[C@H]1c1ccccc1]

To a mixture of 2-(2-fluoro-4-((1R,2R)-6-hydroxy-2-phenyl-1,2,3,4-tetrahydronaphthalen-1-yl)-5-methoxyphenyl)-2-azaspiro[3.5]nonane-7-carbaldehyde (35.0 mg, 1 Eq, 70.1 μmol), (S)-3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione (30.0 mg, 1,2 Eq, 84.1 μmol), TEA (10.6 mg, 0.13 mmol, 1.5 eq.) in DCM (4.0 mL) was added acetic acid (7.6 mg, 0.11 mmol, 1.8 eq.) followed by sodium triacetoxyborohydride (29.7 mg, 20.8 μL, 2 Eq, 140 μmol).

The mixture was stirred at room temperature for 50 minutes and concentrated. The residue was purified by reverse-phase chromatography (acetonitrile/0.05% formic acid) to afford (S)-3-((S)-3-((2-(2-fluoro-4-((1R,2R)-6-hydroxy-2-phenyl-1,2,3,4-tetrahydronaphthalen-1-yl)-5-methoxyphenyl)-2-azaspiro[3.5]nonan-7-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (24.24 mg, 28.86 μmol, 41,2%) as white solid.

LC-MS purity: 100% (UV at 254 nm),840.5 [M+H]⁺.

¹H NMR (400 MHZ, DMSO) δ 10.93 (s, 1H), 8.37 (s, 1H), 7.12-7.02 (m, 4H), 6.93 (s, 1H), 6.78 (d, J=6.8 Hz, 2H), 6.60-6.53 (m, 2H), 6.48 (d, J=8.4 Hz, 1H), 6.10 (d, J=13.6 Hz, 1H), 5,67 (d, J=8.0 Hz, 1H), 5.02 (dd, J=13.2, 5.2 Hz, 1H), 4.62 (d, J=4.8 Hz, 1H), 4.32-4.11 (m, 3H), 3.94-3.77 (m, 2H), 3.55-3.48 (m, 4H), 3.28-3.13 (m, 4H), 2.92 (s, 3H), 2.90-2,72 (m, 4H), 2.68-2.52 (m, 2H), 2.38-1.97 (m, 6H), 1.87 (d, J=11.6 Hz, 2H), 1.74-1.59 (m, 4H), 1.47-1.39 (m, 2H), 0.97-0.85 (m, 2H),

### Compound A170: (S)-3-((S)-3-((1-(2-fluoro-4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

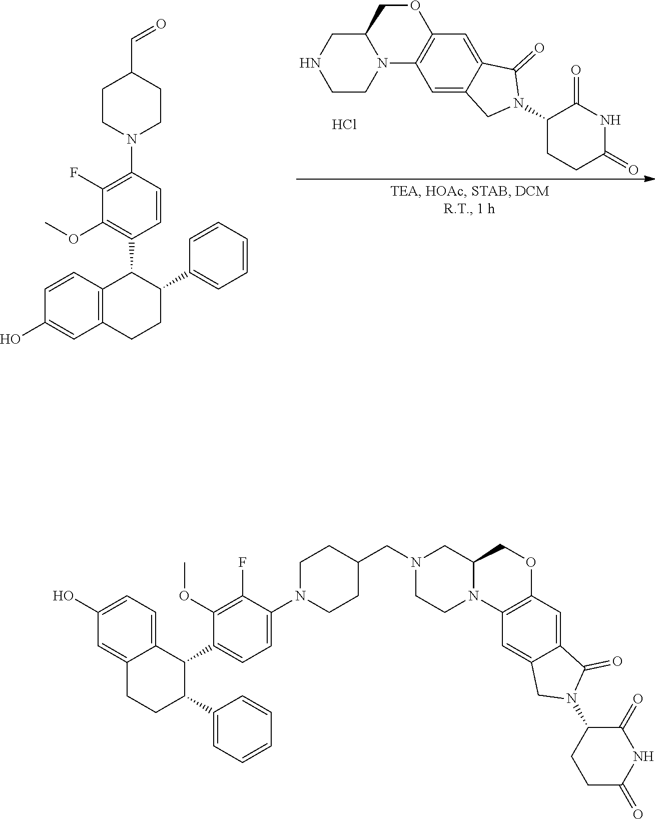 [SMILES: COc1c([C@H]2c3ccc(O)cc3CC[C@H]2c2ccccc2)ccc(N2CCC(C=O)CC2)c1F ; O=C1CC[C@H](N2Cc3cc4c(cc3C2=O)OC[C@@H]2CNCCN42)C(=O)N1 ; Cl ; COc1c([C@H]2c3ccc(O)cc3CC[C@H]2c2ccccc2)ccc(N2CCC(CN3CCN4c5cc6c(cc5OC[C@@H]4C3)C(=O)N([C@H]3CCC(=O)NC3=O)C6)CC2)c1F]

To a mixture of 1-(2-fluoro-4-((1R,2R)-6-hydroxy-2-phenyl-1,2,3,4-tetrahydronaphthalen-1-yl)-3-methoxyphenyl)piperidine-4-carbaldehyde (30 mg, 0.065 mmol, 1 eq.), (S)-3-((S)-8-oxo-1,2,3,4,4a,5,8,10-octahydro-9H-pyrazino[11,2′: 4,5][1,4]oxazino[2,3-f]isoindol-9-yl)piperidine-2,6-dione hydrochloride (31 mg, 0.078 mmol, 1,2 eq.), TEA (10 mg, 0.097 mmol, 1.5 eq.) in DCM (5.0 mL) was added acetic acid (7 mg, 0.11 mmol, 1.7 eq.) followed by sodium triacetoxyborohydride (28 mg, 0.13 mmol, 2 eq.). The mixture was stirred at room temperature for 30 minutes and concentrated. The residue was purified by reverse-phase chromatography (acetonitrile/0.05% formic acid) to afford (S)-3-((S)-3-((1-(2-fluoro-4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (22.77 mg, 43.8% yield) as white solid. LC-MS purity: 100% (UV at 254 nm), 800.7 [M+H]⁺.

¹H NMR (400 MHZ, DMSO) δ 10.94 (s, 1H), 7.15-7.02 (m, 4H), 6.94 (s, 1H), 6.77 (d, J=6.6 Hz, 2H), 6.61-6.53 (m, 3H), 6.49-6.37 (m, 2H), 5.10-4.96 (m, 1H), 4.60 (d, J=5.2 Hz, 1H), 4.33-4.10 (m, 3H), 3.93-3.76 (m, 2H), 3.31-3.12 (m, 5H), 3.02-2.85 (m, 8H), 2.82-2.73 (m, 1H), 2.64-2.52 (m, 3H), 2.41-2.16 (m, 4H), 2.14-2.05 (m, 1H), 2.00-1.91 (m, 1H), 1.84-1.59 (m, 5H), 1.32-1.17 (m, 2H),

### Compound A171: (S)-3-((S)-3-((1-(2-fluoro-4-((1S,2S)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

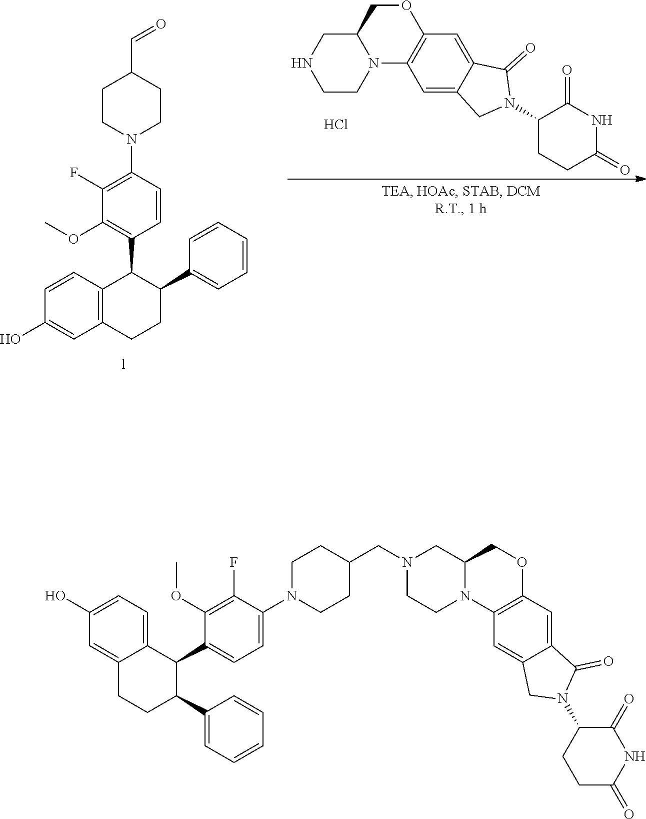 [SMILES: COc1c([C@@H]2c3ccc(O)cc3CC[C@@H]2c2ccccc2)ccc(N2CCC(C=O)CC2)c1F ; O=C1CC[C@H](N2Cc3cc4c(cc3C2=O)OC[C@@H]2CNCCN42)C(=O)N1 ; Cl ; COc1c([C@@H]2c3ccc(O)cc3CC[C@@H]2c2ccccc2)ccc(N2CCC(CN3CCN4c5cc6c(cc5OC[C@@H]4C3)C(=O)N([C@H]3CCC(=O)NC3=O)C6)CC2)c1F]

To a mixture of 1-(2-fluoro-4-((1S,2S)-6-hydroxy-2-phenyl-1,2,3,4-tetrahydronaphthalen-1-yl)-3-methoxyphenyl)piperidine-4-carbaldehyde (30 mg, 0.065 mmol, 1 eq.), (S)-3-((S)-8-oxo-1,2,3,4,4a,5,8,10-octahydro-9H-pyrazino[11,2′: 4,5][1,4]oxazino[2,3-f]isoindol-9-yl)piperidine-2,6-dione hydrochloride (31 mg, 0.078 mmol, 1,2 eq.), TEA (10 mg, 0.097 mmol, 1.5 eq.) in DCM (5.0 mL) was added acetic acid (7 mg, 0.11 mmol, 1.7 eq.) followed by sodium triacetoxyborohydride (28 mg, 0.13 mmol, 2 eq.). The mixture was stirred at room temperature for 30 minutes and concentrated. The residue was purified by reverse-phase chromatography (acetonitrile/0.05% formic acid) to afford (S)-3-((S)-3-((1-(2-fluoro-4-((1S,2S)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (25.01 mg, 48.1% yield) as white solid. LC-MS purity: 100% (UV at 254 nm), 800.7 [M+H]⁺.

¹H NMR (400 MHZ, DMSO) § 10.94 (s, 1H), 7.14-7.01 (m, 4H), 6.94 (s, 1H), 6.77 (d, J=6.6 Hz, 2H), 6.61-6.53 (m, 3H), 6.49-6.38 (m, 2H), 5.08-4.96 (m, 1H), 4.60 (d, J=5.4 Hz, 1H), 4.34-4.08 (m, 3H), 3.95-3.75 (m, 2H), 3.31-3.13 (m, 5H), 3.03-2.84 (m, 8H), 2.82-2.73 (m, 1H), 2.63-2.52 (m, 3H), 2.41-2.15 (m, 4H), 2.13-2.03 (m, 1H), 2.01-1.91 (m, 1H), 1.84-1.58 (m, 5H), 1.32-1.16 (m, 2H),

### Compound A172: (S)-3-(1′-((2-(2-fluoro-4-((1R,2R)-6-hydroxy-2-phenyl-1,2,3,4-tetrahydronaphthalen-1-yl)-5-methoxyphenyl)-2-azaspiro[3.5]nonan-7-yl)methyl)-7-oxo-5,7-dihydro-2H,6H-spiro[furo[2,3-f]isoindole-3,4′-piperidin]-6-yl)piperidine-2,6-dione

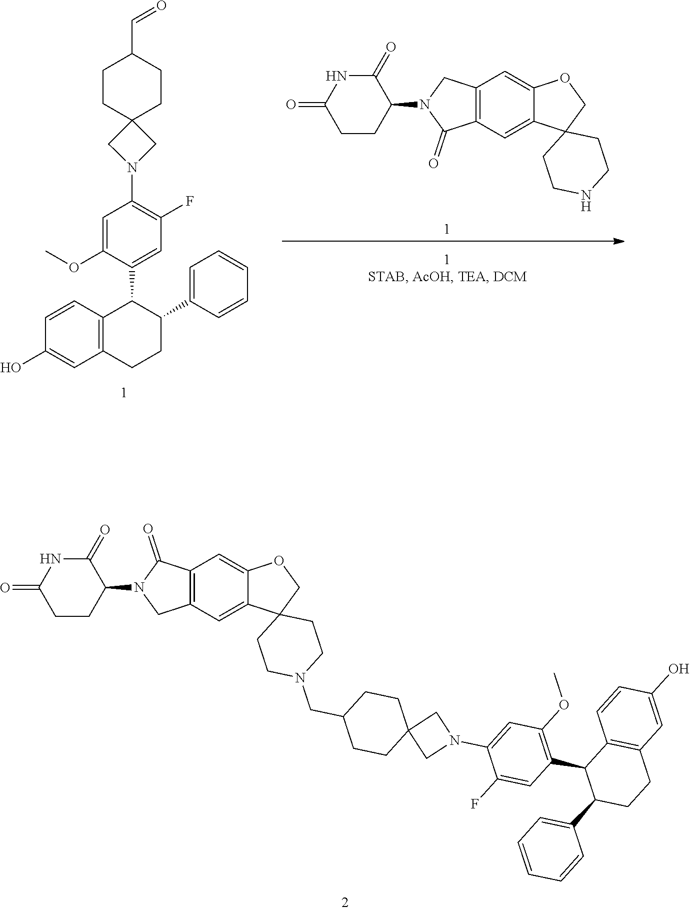 [SMILES: COc1cc(N2CC3(CCC(C=O)CC3)C2)c(F)cc1[C@H]1c2ccc(O)cc2CC[C@H]1c1ccccc1 ; O=C1CC[C@H](N2Cc3cc4c(cc3C2=O)C2(CCNCC2)CO4)C(=O)N1 ; COc1cc(N2CC3(CCC(CN4CCC5(CC4)COc4cc6c(cc45)CN([C@H]4CCC(=O)NC4=O)C6=O)CC3)C2)c(F)cc1[C@H]1c2ccc(O)cc2CC[C@H]1c1ccccc1]

To a mixture of 2-(2-fluoro-4-((1R,2R)-6-hydroxy-2-phenyl-1,2,3,4-tetrahydronaphthalen-1-yl)-5-methoxyphenyl)-2-azaspiro[3.5]nonane-7-carbaldehyde (0.025 g, 1 Eq, 50 μmol), (S)-3-(7-oxo-5,7-dihydro-2H,6H-spiro[furo[2,3-f]isoindole-3,4′-piperidin]-6-yl)piperidine-2,6-dione (21 mg, 1,2 Eq, 60 μmol), TEA (7.6 mg, 0.075 mmol, 1.5 eq.) in DCM (4.0 mL) was added acetic acid (5.4 mg, 0.09 mmol, 1.8 eq.) followed by sodium triacetoxyborohydride (21 mg, 15 μL, 2 Eq, 0.10 mmol). The mixture was stirred at room temperature for 50 minutes and concentrated. The residue was purified by reverse-phase chromatography (acetonitrile/0.05% formic acid) to afford (S)-3-(1′-((2-(2-fluoro-4-((1R,2R)-6-hydroxy-2-phenyl-1,2,3,4-tetrahydronaphthalen-1-yl)-5-methoxyphenyl)-2-azaspiro[3.5]nonan-7-yl)methyl)-7-oxo-5,7-dihydro-2H,6H-spiro[furo[2,3-f]isoindole-3,4′-piperidine]-6-yl)piperidine-2,6-dione (13.88 mg, 16.54 μmol, 33%) (13.88 mg, 33% yield) as white solid. LC-MS purity: 100% (UV at 254 nm), 839.6 [M+H]⁺.

¹H NMR (400 MHZ, DMSO) δ 10.96 (s, 1H), 8.32 (s, 1H), 7.45 (s, 1H), 7.16-6.96 (m, 5H), 6.78 (d, J=6.8 Hz, 2H), 6.58-6.46 (m, 3H), 6.10 (d, J=13.6 Hz, 1H), 5,68 (d, J=8.0 Hz, 1H), 5.07 (dd, J=13.2, 4.8 Hz, 1H), 4.62 (d, J=4.8 Hz, 1H), 4.45 (s, 2H), 4.34 (d, J=17.2 Hz, 1H), 4.21 (d, J=16.8 Hz, 1H), 2.93 (s, 3H), 2.83-2,78 (m, 2H), 2.70-2.54 (m, 2H), 2.48-2.24 (m, 2H), 2.21-2.06 (m, 4H), 2.00-1.85 (m, 8H), 1.73-1.59 (m, 6H), 1.54-1.34 (m, 4H), 0.95-0.87 (m, 2H).

### Compounds A¹⁷³, A¹⁷⁴, A¹⁷⁵, and A¹⁷⁶ were prepared according to the following reaction schemes

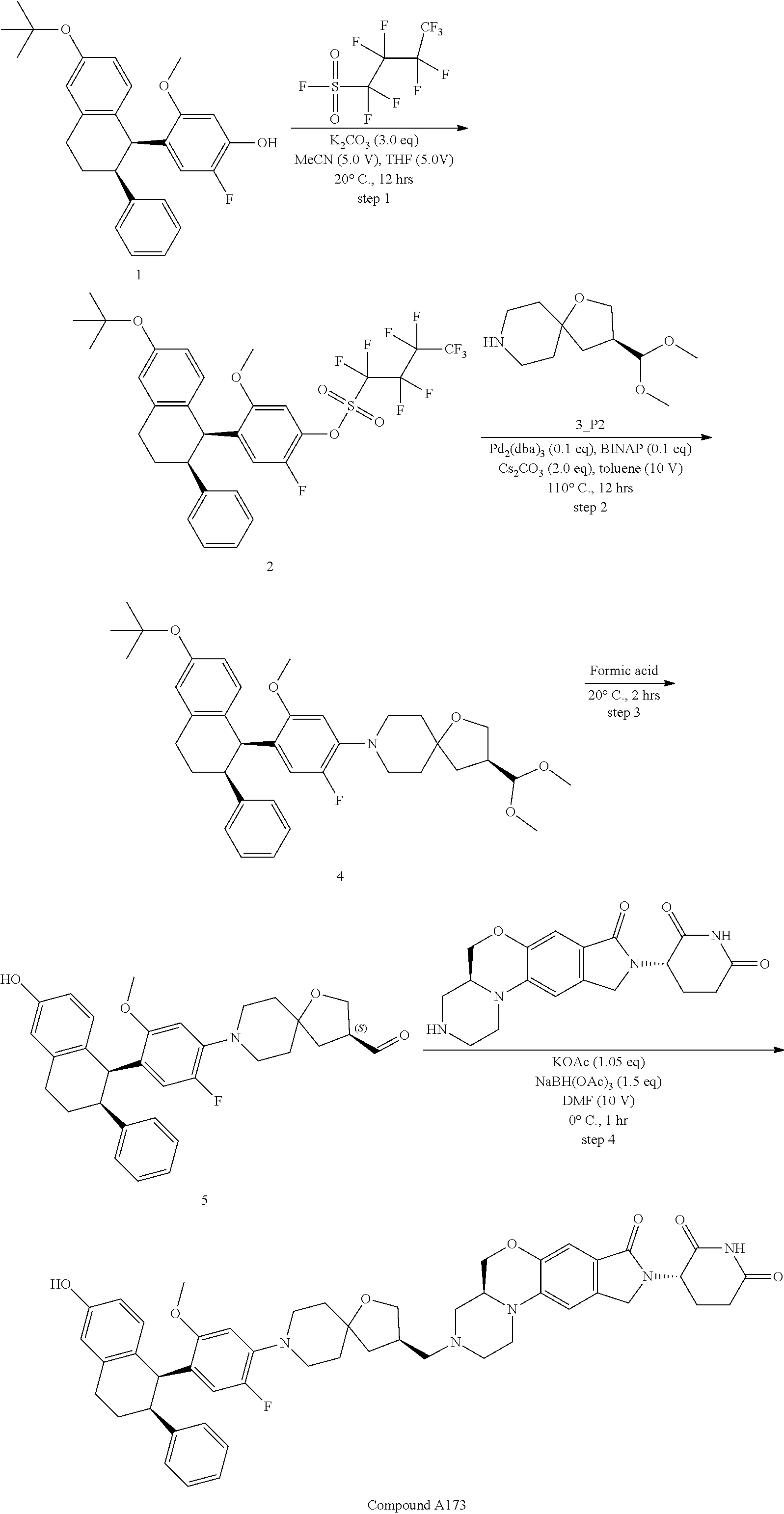

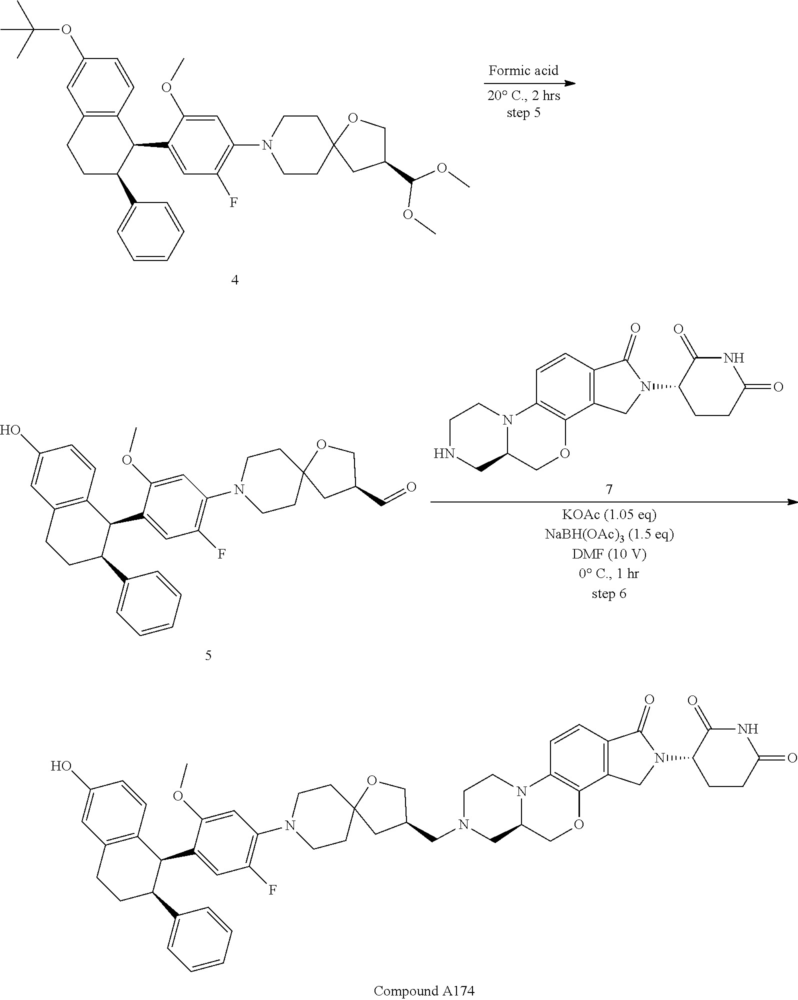 [SMILES: COc1cc(N2CCC3(CC2)C[C@H](C(OC)OC)CO3)c(F)cc1[C@@H]1c2ccc(OC(C)(C)C)cc2CC[C@@H]1c1ccccc1 ; COc1cc(N2CCC3(CC2)C[C@H](C=O)CO3)c(F)cc1[C@@H]1c2ccc(O)cc2CC[C@@H]1c1ccccc1 ; O=C1CC[C@H](N2Cc3c(ccc4c3OC[C@H]3CNCCN43)C2=O)C(=O)N1 ; COc1cc(N2CCC3(CC2)C[C@H](CN2CCN4c5ccc6c(c5OC[C@H]4C2)CN([C@H]2CCC(=O)NC2=O)C6=O)CO3)c(F)cc1[C@@H]1c2ccc(O)cc2CC[C@@H]1c1ccccc1]

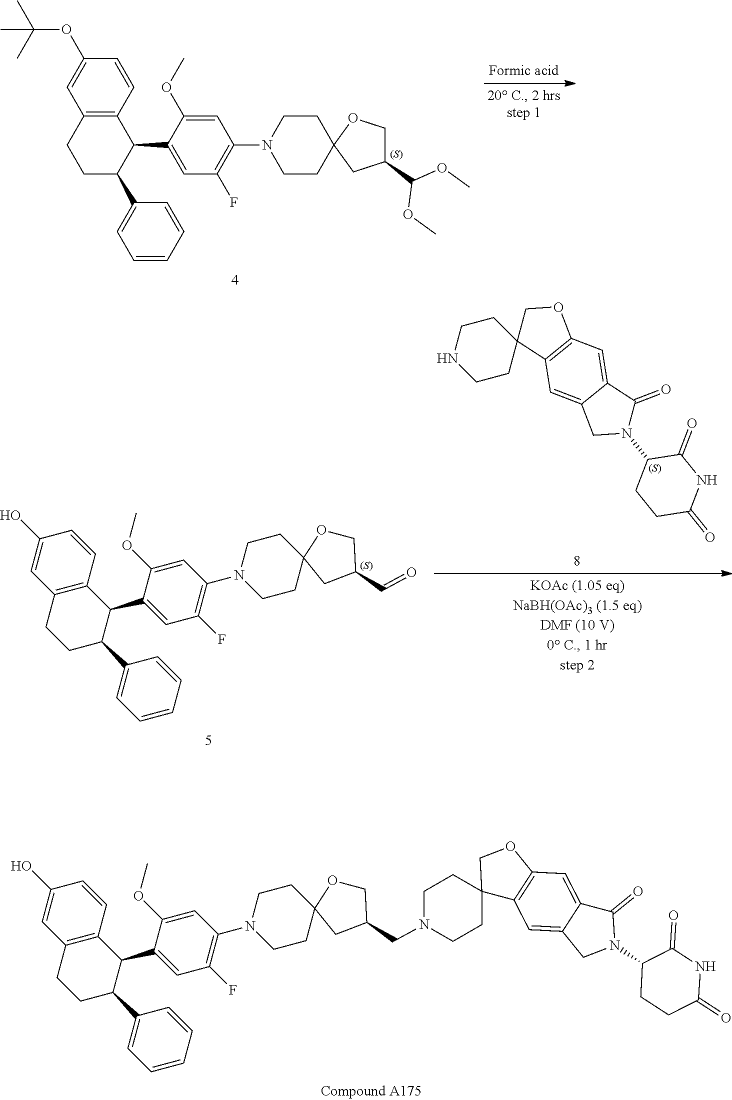

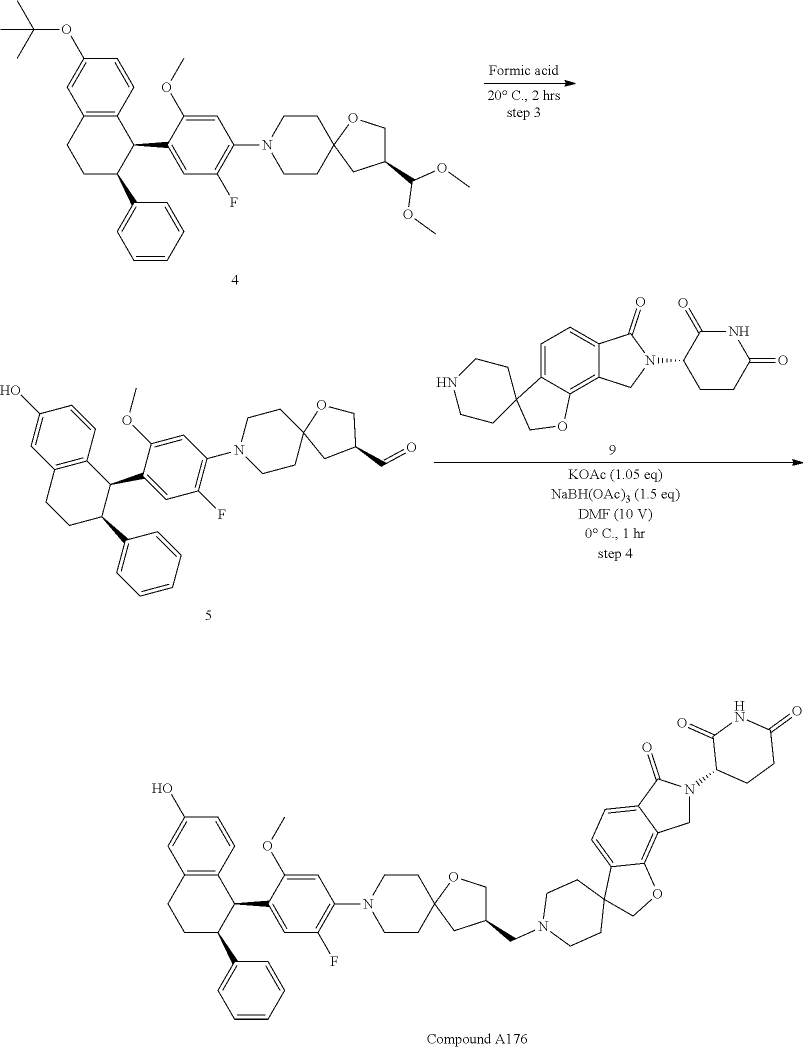 [SMILES: COc1cc(N2CCC3(CC2)C[C@H](C(OC)OC)CO3)c(F)cc1[C@@H]1c2ccc(OC(C)(C)C)cc2CC[C@@H]1c1ccccc1 ; COc1cc(N2CCC3(CC2)C[C@H](C=O)CO3)c(F)cc1[C@@H]1c2ccc(O)cc2CC[C@@H]1c1ccccc1 ; O=C1CC[C@H](N2Cc3c(ccc4c3OCC43CCNCC3)C2=O)C(=O)N1 ; COc1cc(N2CCC3(CC2)C[C@H](CN2CCC4(CC2)COc2c4ccc4c2CN([C@H]2CCC(=O)NC2=O)C4=O)CO3)c(F)cc1[C@@H]1c2ccc(O)cc2CC[C@@H]1c1ccccc1]

### Step 1: Synthesis of 4-((1S,2S)-6-(tert-butoxy)-2-phenyl-1,2,3,4-tetrahydronaphthalen-1-yl)-2-fluoro-5-methoxyphenyl 1,1,2,2,3,3,4,4,4-nonafluorobutane-1-sulfonate

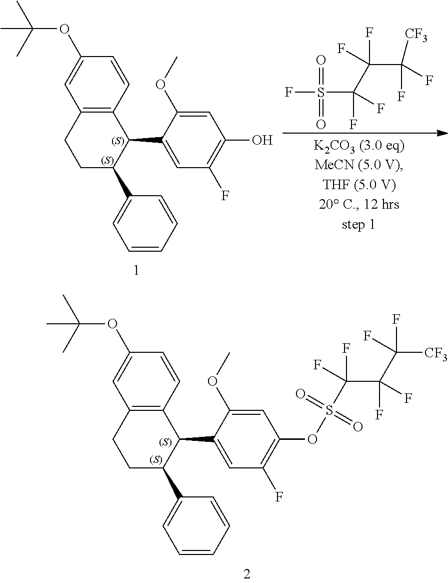

To a solution of compound 1 (4.5 g, 10.70 mmol, 1 eq) in MeCN (45 mL) and THF (45 mL) was added K₂CO₃ (2.22 g, 16.05 mmol, 1.5 eq) and 1,1,2,2,3,3,4,4,4-nonafluorobutane-1-sulfonyl fluoride (4.85 g, 16.05 mmol, 2.82 mL, 1.5 eq) at 20° C. The mixture was stirred at 20° C. for 12 hours and turned to a light yellow suspension. TLC (Petroleum ether/Ethyl acetate=10/1, R_{f}=0.20) indicated compound 1 was consumed completely and one new spot formed. The reaction mixture was filtered and concentrated under reduced pressure to give the crude product. Compound 2 (8 g, crude) was obtained as a light yellow solid.

### Step 2: Synthesis of(S)-8-(4-((1S,2S)-6-(tert-butoxy)-2-phenyl-1,2,3,4-tetrahydronaphthalen-1-yl)-2-fluoro-5-methoxyphenyl)-3-(dimethoxymethyl)-1-oxa-8-azaspiro[4.5]decane

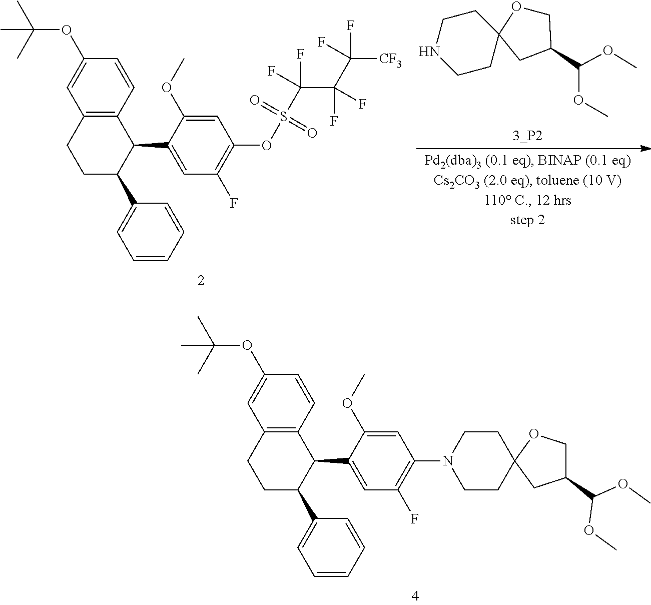 [SMILES: COc1cc(OS(=O)(=O)C(F)(F)C(F)(F)C(F)(F)C(F)(F)F)c(F)cc1[C@@H]1c2ccc(OC(C)(C)C)cc2CC[C@@H]1c1ccccc1 ; COC(OC)[C@@H]1COC2(CCNCC2)C1 ; COc1cc(N2CCC3(CC2)C[C@H](C(OC)OC)CO3)c(F)cc1[C@@H]1c2ccc(OC(C)(C)C)cc2CC[C@@H]1c1ccccc1]

To a solution of compound 2 (2 g, 2.85 mmol, 1 eq) in toluene (20 mL) was added compound 3_P2 (919.25 mg, 4.27 mmol, 1.5 eq), Pd₂(dba) 3 (260.67 mg, 284.66 μmol, 0.1 eq), BINAP (177.25 mg, 284.66 μmol, 0.1 eq) and Cs₂CO₃ (1.85 g, 5,69 mmol, 2 eq). Then the mixture was stirred at 110° C. for 12 hrs. TLC (Petroleum ether/Ethyl acetate=5/1) showed that a little of compound 2 was consumed and the desired product was detected. The mixture was filtered, and the organic layers was concentrated under reduced pressure to give a residue. The residue was purified by column chromatography on silica gel (petroleum ether/EtOAc=30/1 to 5/1). The desired product compound 4 (0.8 g, 1,29 mmol, 45.49% yield, 100% purity) was obtained as yellow oil.

### Step 3: Synthesis of(S)-8-(2-fluoro-4-((1S,2S)-6-hydroxy-2-phenyl-1,2,3,4-tetrahydronaphthalen-1-yl)-5-methoxyphenyl)-1-oxa-8-azaspiro[4.5]decane-3-carbaldehyde

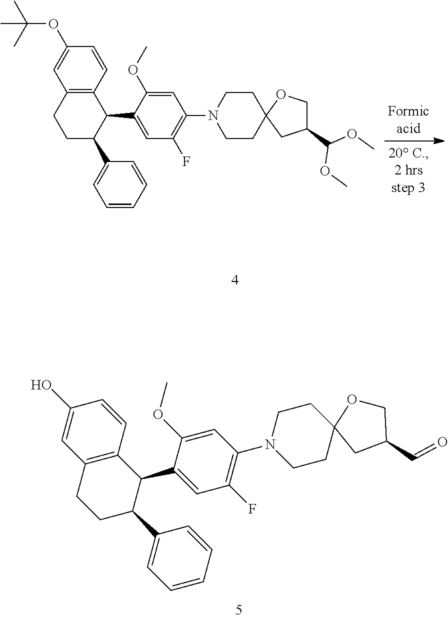 [SMILES: COc1cc(N2CCC3(CC2)C[C@H](C(OC)OC)CO3)c(F)cc1[C@@H]1c2ccc(OC(C)(C)C)cc2CC[C@@H]1c1ccccc1 ; COc1cc(N2CCC3(CC2)C[C@H](C=O)CO3)c(F)cc1[C@@H]1c2ccc(O)cc2CC[C@@H]1c1ccccc1]

A solution of compound 4 (0.1 g, 161.87 μmol, 1 eq) in formic acid (2 mL) was stirred at 20° C. for 1 hr. LCMS showed that compound 4 was consumed and the desired product was detected. The mixture was concentrated under reduced pressure to give a residue. The desired product compound 5 (0.35 g, crude) was obtained as a yellow oil, m/z+1=516.4

### Step 3: Synthesis of(S)-3-((S)-3-(((R)-8-(2-fluoro-4-((1S,2S)-6-hydroxy-2-phenyl-1,2,3,4-tetrahydronaphthalen-1-yl)-5-methoxyphenyl)-1-oxa-8-azaspiro[4.5]decan-3-yl)methyl)-8-oxo-1,2,3,4,4a,5,8,10-octahydro-9H-pyrazino[11,2′: 4,5][1,4]oxazino[2,3-f]isoindol-9-yl)piperidine-2,6-dione (compound A173)

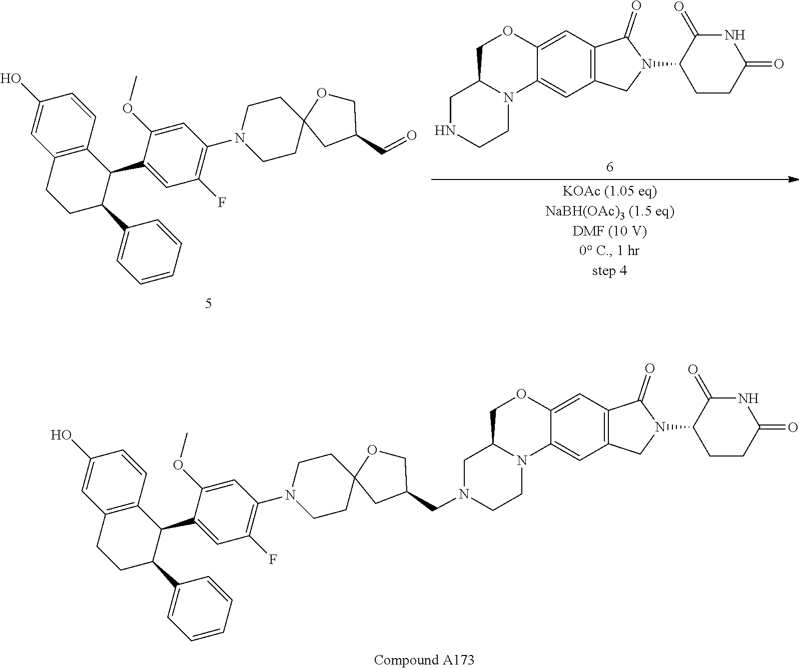

To a solution of compound 6 (64.76 mg, 164.85 μmol, 1.0 eq, HCl) in DMF (1 mL) was added KOAc (16.99 mg, 173.09 μmol, 1.05 eq) to the mixture at 20° C. Then the mixture was cooled to 0° C., and NaBH (OAc) 3 (52.41 mg, 247.28 μmol, 1.5 eq) was added to the mixture. A solution compound 5 (0.085 g, 164.85 μmol, 1 eq) in formic acid (0.3 mL) was added at 0° C., and then the mixture was stirred at 0° C. for 5 hrs. LCMS showed that compound 5 was consumed and the desired product was detected. The mixture was quenched with water (0.2 mL). The residue was purified by pre-HPLC(column: Phenomenex C18 75*30 mm*3 um; mobile phase: [water (FA)-ACN]; B %: 35%-60%, 8 min). The desired product (0.073 g, 84.08 μmol, 51.00% yield, 98.59% purity) was obtained as a white solid, ¹H NMR (400 MHZ, DMSO-d₆) 10.93 (br s, 1H), 9.15 (br s, 1H), 7.13-7.02 (m, 4H), 6.94 (s, 1H), 6.78-6.72 (m, 2H), 6.61-6.53 (m, 2H), 6.48 (dd, J=2,3, 8.4 Hz, 1H), 6.24-6.14 (m, 2H), 5.03 (dd, J=5.2, 13.3 Hz, 1H), 4.67 (br d, J=5.1 Hz, 1H), 4.32-4,22 (m, 2H), 4.19-4.11 (m, 1H), 3.95-3.86 (m, 2H), 3.82 (br d, J=11.6 Hz, 1H), 3.19 (br d, J=9.1 Hz, 2H), 3.05-2.84 (m, 13H), 2.81-2,73 (m, 1H), 2.62-2.54 (m, 2H), 2.41-2.30 (m, 3H), 2.21-2.05 (m, 2H), 1.97 (br dd, J=7.9, 11.8 Hz, 2H), 1.77-1.57 (m, 6H), 1.37 (br dd, J=8.3, 12,3 Hz, 1H), m/z+1=856.3

### Step 4: Synthesis of(S)-3-((R)-7-(((R)-8-(2-fluoro-4-((1S,2S)-6-hydroxy-2-phenyl-1,2,3,4-tetrahydronaphthalen-1-yl)-5-methoxyphenyl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (compound A174)

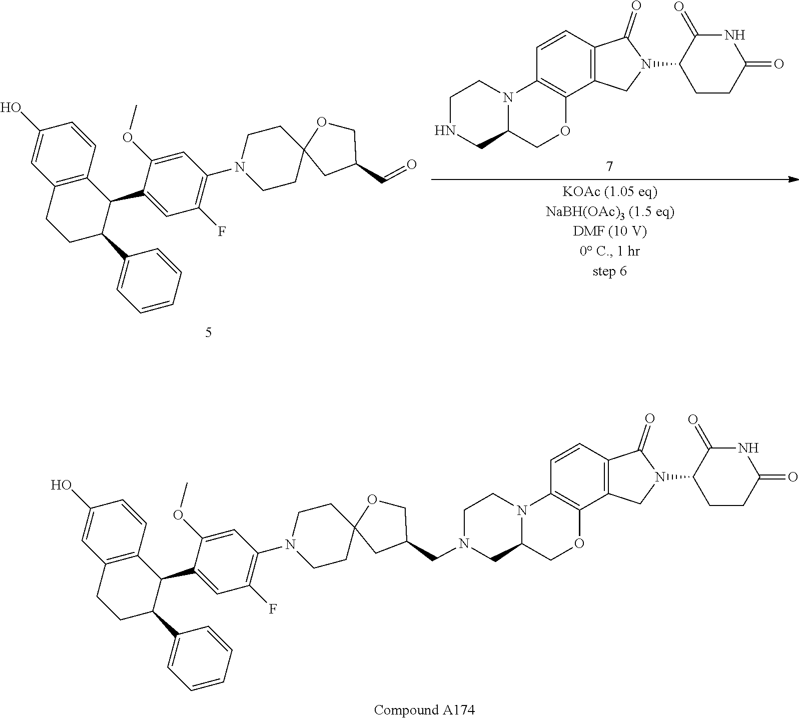 [SMILES: COc1cc(N2CCC3(CC2)C[C@H](C=O)CO3)c(F)cc1[C@@H]1c2ccc(O)cc2CC[C@@H]1c1ccccc1 ; O=C1CC[C@H](N2Cc3c(ccc4c3OC[C@H]3CNCCN43)C2=O)C(=O)N1 ; COc1cc(N2CCC3(CC2)C[C@H](CN2CCN4c5ccc6c(c5OC[C@H]4C2)CN([C@H]2CCC(=O)NC2=O)C6=O)CO3)c(F)cc1[C@@H]1c2ccc(O)cc2CC[C@@H]1c1ccccc1]

To a solution of compound 7 (64.76 mg, 164.85 μmol, 1.0 eq, HCl) in DMF (1 mL) was added KOAc (16.99 mg, 173.09 μmol, 1.05 eq) at 20° C. The mixture was cooled to 0° C., and then NaBH (OAc) 3 (52.41 mg, 247.28 μmol, 1.5 eq) was added to the mixture. Then a solution of compound 5 (0.085 g, 164.85 μmol, 1 eq) in formic acid (0.3 mL) was added to the mixture at 0° C. The mixture was stirred at 0° C. for 2 hrs. LCMS showed that compound 5 was consumed and the desired product was detected. The mixture was quenched with water (0.5 mL). The mixture was purified by pre-HPLC(column: Phenomenex C18 75*30 mm*3 um; mobile phase: [water (FA)-ACN]; B %: 35%-60%, 8 min). The desired product (0.083 g, 96.96 μmol, 58.82% yield) was obtained as a white solid, ¹H NMR (400 MHZ, DMSO-d₆) 10.94 (br s, 1H), 9.16 (br s, 1H), 7.18 (d, J=8.3 Hz, 1H), 7.14-6.98 (m, 4H), 6.75 (br d, J=6.3 Hz, 2H), 6.63-6.54 (m, 2H), 6.52-6.44 (m, 1H), 6.28-6.14 (m, 2H), 5.03 (dd, J=5.0, 13.3 Hz, 1H), 4.67 (br d, J=5.1 Hz, 1H), 4.36 (br d, J=9.3 Hz, 1H), 4.31-4.19 (m, 1H), 4.11 (br d, J=16.9 Hz, 1H), 4.01-3.78 (m, 3H), 3.19 (br d, J=8.5 Hz, 3H), 3.05-2.83 (m, 13H), 2.80-2,71 (m, 1H), 2.63-2.55 (m, 2H), 2.41-2.27 (m, 2H). 2.13 (br t, J=11.3 Hz, 2H), 1.96 (br dd, J=7.3, 12.0 Hz, 2H), 1.77-1.56 (m, 6H), 1.37 (br dd, J=8.3, 12.4 Hz, 1H), m/z+1=856.3

### Step 5: Synthesis of(S)-3-(1′-(((R)-8-(2-fluoro-4-((1S,2S)-6-hydroxy-2-phenyl-1,2,3,4-tetrahydronaphthalen-1-yl)-5-methoxyphenyl)-1-oxa-8-azaspiro[4.5]decan-3-yl)methyl)-7-oxo-5,7-dihydro-2H,6H-spiro[furo[2,3-f]isoindole-3,4′-piperidin]-6-yl)piperidine-2,6-dione (Compound A175)

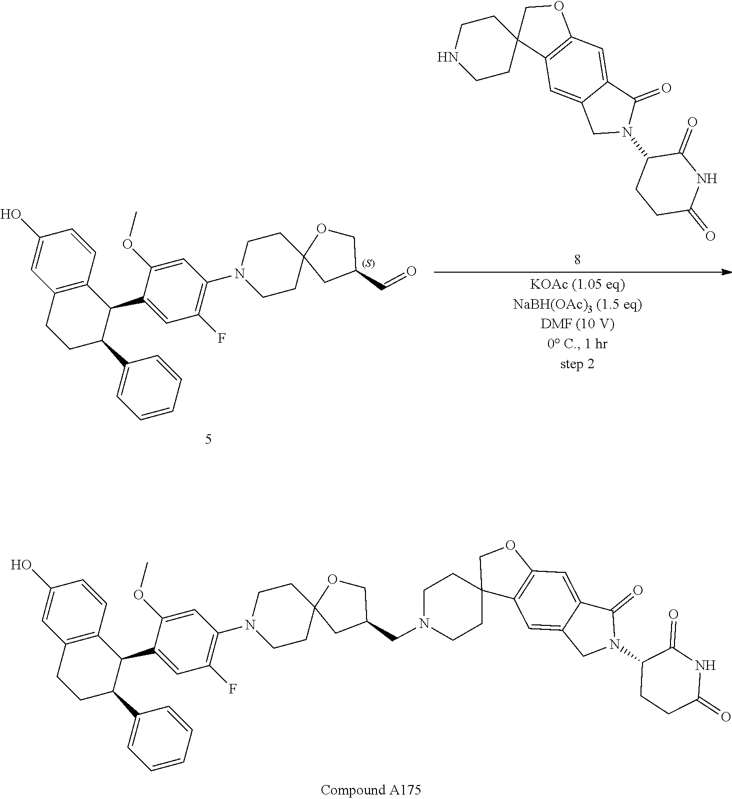

To a solution of compound 8 (76.00 mg, 193.94 μmol, 1.0 eq, HCl) in DMF (2 mL) was added KOAc (19.99 mg, 203.64 μmol, 1.05 eq) at 20° C. The mixture was cooled to 0° C., and then NaBH (OAc) 3 (61.66 mg, 290.92 μmol, 1.5 eq) was added to the mixture. Then a solution of compound 5 (0.1 g, 193.94 μmol, 1 eq) in formic acid (0.5 mL) was added at 0° C. The mixture was stirred at 0° C. for 5 hrs. LCMS showed that compound 5 was consumed and the desired product was detected. The mixture was quenched with water (0.2 mL). The mixture was purified by pre-HPLC(column: Phenomenex Luna C18 75*30 mm*3 um; mobile phase: [water (FA)-ACN]; B %: 50%-90%, 8 min). The desired product (0.057 g, 66.67 μmol, 34.37% yield) was obtained as a white solid, ¹H NMR (400 MHZ, DMSO-d₆) 10.97 (br s, 1H), 9.74-8.67 (m, 1H), 7.46 (br s, 1H), 7.20-6.99 (m, 4H), 6.76 (br d, J=5.8 Hz, 2H), 6.65-6.40 (m, 3H), 6.20 (br d, J=10.3 Hz, 2H), 5.08 (br d, J-8.8 Hz, 1H), 4.68 (br s, 1H), 4.46 (br s, 2H), 4.37-4.30 (m, 1H), 4.28-4.14 (m, 1H), 3.88 (br s, 1H), 3.28 (br s, 1H), 3.05-2,78 (m, 12H), 2.59 (br d, J=16.0 Hz, 2H), 2.42-2.28 (m, 3H), 2.16 (br d, J=7.3 Hz, 1H), 2.08-1.82 (m, 7H), 1.66 (br s, 7H), 1.38 (br d, J=8.4 Hz, 1H), m/z+1=855.3

### Step 6: Synthesis of(S)-3-(1′-(((R)-8-(2-fluoro-4-((1S,2S)-6-hydroxy-2-phenyl-1,2,3,4-tetrahydronaphthalen-1-yl)-5-methoxyphenyl)-1-oxa-8-azaspiro[4.5]decan-3-yl)methyl)-6-oxo-6,8-dihydro-2H,7H-spiro[furo[2,3-e]isoindole-3,4′-piperidin]-7-yl)piperidine-2,6-dione (compound A176)

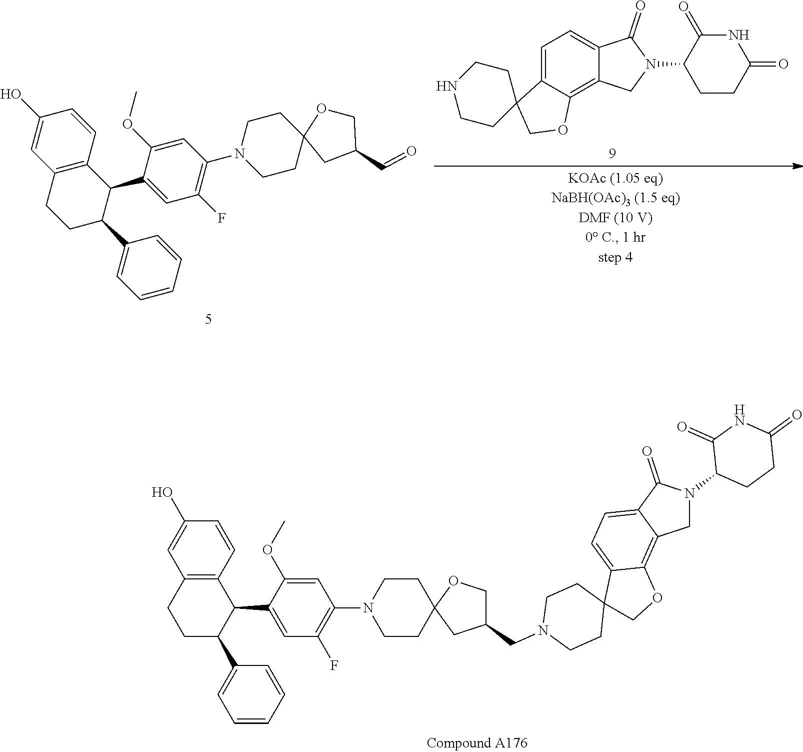 [SMILES: COc1cc(N2CCC3(CC2)C[C@H](C=O)CO3)c(F)cc1[C@@H]1c2ccc(O)cc2CC[C@@H]1c1ccccc1 ; O=C1CC[C@H](N2Cc3c(ccc4c3OCC43CCNCC3)C2=O)C(=O)N1 ; COc1cc(N2CCC3(CC2)C[C@H](CN2CCC4(CC2)COc2c4ccc4c2CN([C@H]2CCC(=O)NC2=O)C4=O)CO3)c(F)cc1[C@@H]1c2ccc(O)cc2CC[C@@H]1c1ccccc1]

To a mixture of compound 9 (64.6 mg, 164.85 μmol, 1.0 eq, HCl) in DMF (2 mL) was added KOAc (16.9 mg, 173 μmol, 1.05 eq) at 20° C. The mixture was cooled to 0° C., and then NaBH (OAc) 3 (52.4 mg, 247 μmol, 1.5 eq) was added to the mixture. Then a solution of compound 5 (0.085 g, 164 μmol, 1 eq) in formic acid (0.6 mL) was added at 0° C. The mixture was stirred at 0° C. for 5 hrs. LCMS showed that compound 5 was consumed and the desired product was detected. The mixture was quenched with water (0.5 mL). The mixture was purified by pre-HPLC (column: Phenomenex Luna C18 75*30 mm*3 um; mobile phase: [water (FA)-ACN]; B %: 50%-90%, 8 min). The desired product (0.053 g, 61.39 μmol, 37.24% yield, 99.04% purity) was obtained as a white solid, ¹H NMR (400 MHZ, DMSO-d₆) 10.98 (br s, 1H), 9.54-8.54 (m, 1H), 7.48-7.20 (m, 2H), 7.09 (br s, 3H), 6.76 (br d, J=4.0 Hz, 2H), 6.65-6.41 (m, 3H), 6.20 (br d, J=9.8 Hz, 2H), 5.09 (br d, J=8.6 Hz, 1H), 4.68 (br s, 1H), 4.52 (br s, 2H), 4.38 (br d, J=17.0 Hz, 1H), 4.22 (br d, J=16.8 Hz, 1H), 3.89 (br s, 1H), 3.28 (br s, 1H), 3.02-2,78 (m, 13H), 2.59 (br d, J=16.5 Hz, 2H), 2.45-2.29 (m, 3H), 2.15 (br s, 1H), 1.97 (br d, J=11.9 Hz, 6H), 1.67 (br s, 7H), 1.37 (br s, 1H), m/z+1=855.3

### Compound A208. (3S)-3-[1′-[[(3R)-8-[2-fluoro-4-[(1S,2S)-6-hydroxy-2-phenyl-tetralin-1-yl]-5-methoxy-phenyl]-1-oxa-8-azaspiro[4.5]decan-3-yl]methyl]-5-methyl-6-oxo-spiro[2,8-dihydrofuro[2,3-e]isoindole-3,4′-piperidine]-7-yl]piperidine-2,6-dione

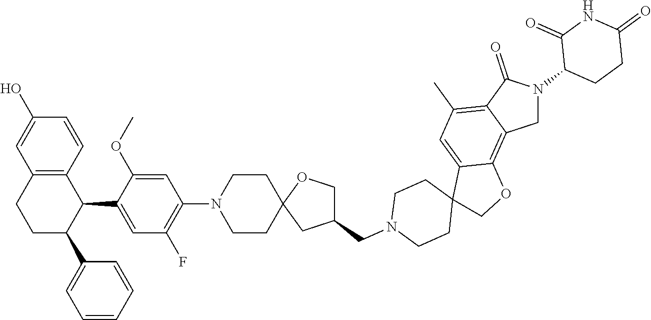 [SMILES: COc1cc(N2CCC3(CC2)C[C@H](CN2CCC4(CC2)COc2c4cc(C)c4c2CN([C@H]2CCC(=O)NC2=O)C4=O)CO3)c(F)cc1[C@@H]1c2ccc(O)cc2CC[C@@H]1c1ccccc1]

### Step 1: Synthesis of (3S)-8-[2-fluoro-4-[(1S,2S)-6-hydroxy-2-phenyl-tetralin-1-yl]-5-methoxy-phenyl]-1-oxa-8-azaspiro[4.5]decane-3-carbaldehyde

(S)-8-(4-((1S,2S)-6-(tert-butoxy)-2-phenyl-1,2,3,4-tetrahydronaphthalen-1-yl)-2-fluoro-5-methoxyphenyl)-3-(dimethoxymethyl)-1-oxa-8-azaspiro[4.5]decane (50.0 mg, 80.9 μmol, 1.00 eq) was stirred in formic acid (1 mL) at 20° C. for 1 hour and turned yellow solution. The mixture was concentrated under reduced pressure to give the product (0.5 mL solution) at 20° C. (3S)-8-[2-fluoro-4-[(1S,2S)-6-hydroxy-2-phenyl-tetralin-1-yl]-5-methoxy-phenyl]-1-oxa-8-azaspiro[4.5]decane-3-carbaldehyde was obtained as a yellow solution, m/c+1=516.3

### Step 2: Synthesis of (3S)-3-[ ]′-[[(3R)-8-[2-fluoro-4-[(1S,2S)-6-hydroxy-2-phenyl-tetralin-1-yl]-5-methoxy-phenyl]-1-oxa-8-azaspiro[4.5]decan-3-yl]methyl]-5-methyl-6-oxo-spiro[2,8-dihydrofuro[2,3-e]isoindole-3,4′-piperidine]-7-yl]piperidine-2,6-dione

To the suspension of(S)-3-(5-methyl-6-oxo-6,8-dihydro-2H,7H-spiro[furo[2,3-c]isoindole-3,4′-piperidin]-7-yl)piperidine-2,6-dione (38.1 mg, 84.5 μmol, 1.05 eq. HCl) in DMF (1 ml.) was added KOAc (8.29 mg, 84.5 μmol, 1.05 eq) in portions at 20° C., and stirred for 10 minutes. The mixture was cooled to 0° C. NaBH (OAc) 3 (25,6 mg, 121 μmol, 1.50 eq) and the solution of (3S)-8-[2-fluoro-4-[(1S,2S)-6-hydroxy-2-phenyl-tetralin-1-yl]-5-methoxy-phenyl]-1-oxa-8-azaspiro[4.5]decane-3-carbaldehyde (41.5 mg, 80.5 μmol, 1.00 eq) in formic acid (0.5 mL) was added in portions at 0° C. The reaction mixture was stirred at 0° C. for 1 hour and turned brown solution. The reaction mixture was purified by prep-HPLC(column: Phenomenex Luna C18 200*40 mm*10 um; mobile phase: [water (FA)-ACN]; B %: 25%-60%, 8 min). (3S)-3-[1′-[[(3R)-8-[2-fluoro-4-[(1S,2S)-6-hydroxy-2-phenyl-tetralin-1-yl]-5-methoxy-phenyl]-1-oxa-8-azaspiro[4.5]decan-3-yl]methyl]-5-methyl-6-oxo-spiro[2,8-dihydrofuro[2,3-e]isoindole-3,4′-piperidine]-7-yl]piperidine-2,6-dione (37.6 mg, 43.3 μmol, 53.8% yield, 100% purity) was obtained as a white solid, which was indicated by HNMR, ¹H NMR (400 MHZ, DMSO-d₆) δ 10.95 (s, 1H), 9.13 (s, 1H), 7.21-7.03 (m, 4H), 6.75 (br d, J=7.8 Hz, 2H), 6.64-6.54 (m, 2H), 6.50-6.41 (m, 1H), 6.23-6.11 (m, 2H), 5.04 (dd, J=5.2, 13.0 Hz, 1H), 4.67 (br d, J=5.3 Hz, 1H), 4.52-4.41 (m, 2H), 4.35-4,25 (m, 1H), 4.14 (d, J=17.0 Hz, 1H), 3.88 (br t. J=7.6 Hz, 1H), 3.45 (br t. J=7.8 Hz, 1H), 3.24 (br s, 1H), 3.03-2,77 (m, 13H), 2.60 (br s, 2H), 2.54 (s, 3H), 2.33 (br s, 2H), 2.15 (br dd, J=6.9, 12.7 Hz, 1H), 2.03-1.82 (m, 7H), 1.64 (br s, 7H), m/c+1-869.4.

### Compound A209. (3S)-3-[1′-[(3S)-9-[4-[(1R,2S)-6-Hydroxy-2-phenyl-tetralin-1-yl]phenyl]-1-oxa-9-azaspiro[5.5]undecan-3-yl]methyl]-5-methyl-6-oxo-spiro[2,8-dihydrofuro[2,3-e]isoindole-3,4′-piperidine]-7-yl]piperidine-2,6-dione

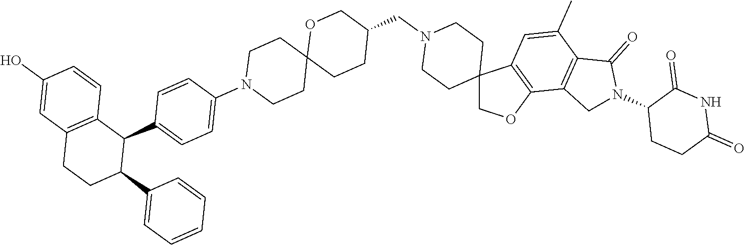 [SMILES: Cc1cc2c(c3c1C(=O)N([C@H]1CCC(=O)NC1=O)C3)OCC21CCN(C[C@@H]2CCC3(CCN(c4ccc([C@@H]5c6ccc(O)cc6CC[C@@H]5c5ccccc5)cc4)CC3)OC2)CC1]

### Step 1: Synthesis of (3R)-9-[4-[(1R,2S)-6-hydroxy-2-phenyl-tetralin-1-yl]phenyl]-1-oxa-9-azaspiro[5.5]undecane-3-carbaldehyde

(R)-9-(4-((1R,2S)-6-(tert-Butoxy)-2-phenyl-1,2,3,4-tetrahydronaphthalen-1-yl)phenyl)-3-(dimethoxymethyl)-1-oxa-9-azaspiro[5.5]undecane (40.0 mg, 68.5 μmol, 1.00 eq) was stirred in formic acid (1 mL) at 20° C. for 1 hour and turned yellow solution. The mixture was concentrated under reduced pressure to give the product (0.5 mL solution) at 20° C. (R)-9-(4-((1R,2S)-6-hydroxy-2-phenyl-1,2,3,4-tetrahydronaphthalen-1-yl)phenyl)-1-oxa-9-azaspiro[5.5]undecane-3-carbaldehyde was obtained as a yellow solution, m/e+1=482.4

### Step 2: Synthesis of (3S)-3-[1′-[[(3S)-9-[4-[(1R,2S)-6-hydroxy-2-phenyl-tetralin-1-yl]phenyl]-1-oxa-9-azaspiro[5.5]undecan-3-yl]methyl]-5-methyl-6-oxo-spiro[2,8-dihydrofuro[2,3-e]isoindole-3,4′-piperidine]-7-yl]piperidine-2,6-dione

To a suspension of(S)-3-(5-methyl-6-oxo-6,8-dihydro-2H,7H-spiro[furo[2,3-e]isoindole-3,4′-piperidin]-7-yl)piperidine-2,6-dione (32.4 mg, 71.9 μmol, 1.05 eq, HCl) in DMF (1 mL) was added KOAc (7.06 mg, 71.9 μmol, 1.05 eq) in portions at 20° C., and stirred for 10 minutes. The mixture was cooled to 0° C. NaBH (OAc) 3 (21.8 mg, 103 μmol, 1.5 eq) and the solution of (R)-9-(4-((1R,2S)-6-hydroxy-2-phenyl-1,2,3,4-tetrahydronaphthalen-1-yl)phenyl)-1-oxa-9-azaspiro[5.5]undecane-3-carbaldehyde (33.0 mg, 68.5 μmol, 1.00 eq) in formic acid (0.5 mL) was added in portions at 0° C. The reaction mixture was stirred at 0° C. for 1 hour and turned brown solution. The reaction mixture was purified by prep-HPLC(column: Phenomenex Luna C18 200*40 mm*10 um; mobile phase: [water (FA)-ACN]; B %: 20%-60%, 8 min). (3S)-3-[1′-[(3S)-9-[4-[(1R,2S)-6-Hydroxy-2-phenyl-tetralin-1-yl]phenyl]-1-oxa-9-azaspiro[5.5]undecan-3-yl]methyl]-5-methyl-6-oxo-spiro[2,8-dihydrofuro[2,3-e]isoindole-3,4′-piperidine]-7-yl]piperidine-2,6-dione (38.1 mg, 45,6 μmol, 66.5% yield, 100% purity) was obtained as a white solid, which was indicated by HNMR, ¹H NMR (400 MHZ, DMSO-d₆) δ 10.95 (s, 1H), 9.10 (br s, 1H), 7.02-7.22 (m, 4H), 6.82 (br d, J=7.13 Hz, 2H), 6.42-6.71 (m, 5H), 6.14-6.26 (m, 2H), 5.03 (br dd, J=13.32, 4.94 Hz, 1H), 4.42-4.50 (m, 2H), 4.24-4.33 (m, 1H), 4.09-4.18 (m, 2H), 3.66 (br dd, J=10.76, 2.63 Hz, 1H), 3.10-3.16 (m, 2H), 2.73-3.03 (m, 9H), 2.56-2.66 (m, 3H), 2.37-2.44 (m, 1H), 1.82-2.26 (m, 10H), 1.47-1.78 (m, 8H), 1.28-1.46 (m, 3H), m/c+1=835.4

### Compound A210. (3S)-3-[1′-[[(3R)-9-[4-[(1R,2S)-6-hydroxy-2-phenyl-tetralin-1-yl]phenyl]-1-oxa-9-azaspiro[5.5]undecan-3-yl]methyl]-5-methyl-6-oxo-spiro[2,8-dihydrofuro[2,3-e]isoindole-3,4′-piperidine]-7-yl]piperidine-2,6-dione

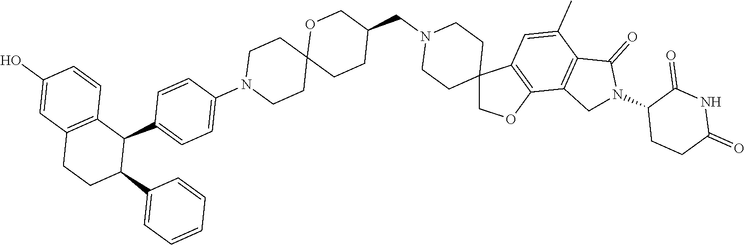 [SMILES: Cc1cc2c(c3c1C(=O)N([C@H]1CCC(=O)NC1=O)C3)OCC21CCN(C[C@H]2CCC3(CCN(c4ccc([C@@H]5c6ccc(O)cc6CC[C@@H]5c5ccccc5)cc4)CC3)OC2)CC1]

### Step 1: Synthesis of (3S)-9-[4-[(1R,2S)-6-hydroxy-2-phenyl-tetralin-1-yl]phenyl]-1-oxa-9-azaspiro[5.5]undecane-3-carbaldehyde

(S)-9-(4-((1R,2S)-6-(tert-butoxy)-2-phenyl-1,2,3,4-tetrahydronaphthalen-1-yl)phenyl)-3-(dimethoxymethyl)-1-oxa-9-azaspiro[5.5]undecane (40.0 mg, 68.5 μmol, 1.00 eq) was stirred in formic acid (1 mL) at 20° C. for 1 hour and turned yellow solution. The mixture was concentrated under reduced pressure to give the product (0.5 mL solution) at 20° C. (3S)-9-[4-[(1R,2S)-6-hydroxy-2-phenyl-tetralin-1-yl]phenyl]-1-oxa-9-azaspiro[5.5]undecane-3-carbaldehyde was obtained as a yellow solution, m/e+1=482.4

### Step 2: Synthesis of (3S)-3-[ ]′-[[(3R)-9-[4-[(1R,2S)-6-hydroxy-2-phenyl-tetralin-1-yl]phenyl]-1-oxa-9-azaspiro[5.5]undecan-3-yl]methyl]-5-methyl-6-oxo-spiro[2,8-dihydrofuro[2,3-e]isoindole-3,4′-piperidine]-7-yl]piperidine-2,6-dione

To the suspension of(S)-3-(5-methyl-6-oxo-6,8-dihydro-2H,7H-spiro[furo[2,3-e]isoindole-3,4′-piperidin]-7-yl)piperidine-2,6-dione (32.4 mg, 71.9 μmol, 1.05 eq, HCl) in DMF (1 mL) was added KOAc (7.06 mg, 71.9 μmol, 1.05 eq) in portions at 20° C., and stirred for 10 minutes. The mixture was cooled to 0° C., NaBH (OAc) 3 (21.8 mg, 103 μmol, 1.50 eq) and the solution of (3S)-9-[4-[(1R,2S)-6-hydroxy-2-phenyl-tetralin-1-yl]phenyl]-1-oxa-9-azaspiro[5.5]undecane-3-carbaldehyde (33.0 mg, 68.5 μmol, 1.00 eq) in formic acid (0.5 mL) was added in portions at 0° C. The reaction mixture was stirred at 0° C. for 1 hour and turned brown solution. The reaction mixture was purified by prep-HPLC(column: Phenomenex Luna C18 200*40 mm*10 um; mobile phase: [water (FA)-ACN]; B %: 20%-60%, 8 min). (3S)-3-[1′-[[(3R)-9-[4-[(1R,2S)-6-hydroxy-2-phenyl-tetralin-1-yl]phenyl]-1-oxa-9-azaspiro[5.5]undecan-3-yl]methyl]-5-methyl-6-oxo-spiro[2,8-dihydrofuro[2,3-e]isoindole-3,4′-piperidine]-7-yl]piperidine-2,6-dione (21.0 mg, 24.7 μmol, 36.0% yield, 98.1% purity) was obtained as a white solid, which was indicated by HNMR, ¹H NMR (400 MHZ, DMSO-d₆) § 10.87-11,13 (m, 1H), 8.97-9.31 (m, 1H), 7.06-7.32 (m, 4H), 6.79-6.97 (m, 2H), 6.44-6.69 (m, 5H), 6.12-6.38 (m, 2H), 4.96-5.15 (m, 1H), 4.41-4.57 (m, 2H), 4.26-4.39 (m, 1H), 4.07-4,22 (m, 2H), 3.66 (br d, J=11,26 Hz, 1H), 3.15 (br dd, J=4.63, 1.88 Hz, 2H), 2.72-3.07 (m, 9H), 2.60-2.68 (m, 2H), 2.31-2.44 (m, 1H), 1.86-2.30 (m, 10H), 1.47-1.79 (m, 9H), 1.24-1.46 (m, 3H), m/e+1=835.4

### Compound A211. (3S)-3-[1′-[[(3S)-8-[2-fluoro-4-[(1S,2S)-6-hydroxy-2-phenyl-tetralin-1-yl]-5-methoxy-phenyl]-1-oxa-8-azaspiro[4.5]decan-3-yl]methyl]-5-methyl-6-oxo-spiro[2,8-dihydrofuro[2,3-e]isoindole-3,4′-piperidine]-7-yl]piperidine-2,6-dione

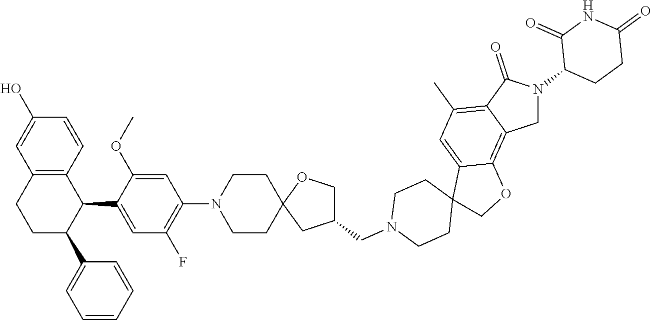 [SMILES: COc1cc(N2CCC3(CC2)C[C@@H](CN2CCC4(CC2)COc2c4cc(C)c4c2CN([C@H]2CCC(=O)NC2=O)C4=O)CO3)c(F)cc1[C@@H]1c2ccc(O)cc2CC[C@@H]1c1ccccc1]

### Step 1: Synthesis of (3R)-8-[2-fluoro-4-[(1S,2S)-6-hydroxy-2-phenyl-tetralin-1-yl]-5-methoxy-phenyl]-1-oxa-8-azaspiro[4.5]decane-3-carbaldehyde

(R)-8-(4-((1S,2S)-6-(tert-butoxy)-2-phenyl-1,2,3,4-tetrahydronaphthalen-1-yl)-2-fluoro-5-methoxyphenyl)-3-(dimethoxymethyl)-1-oxa-8-azaspiro[4.5]decane (50.0 mg, 80.9 μmol, 1.00 eq) was stirred in formic acid (1 mL) at 20° C. for 1 hour and turned yellow solution. The mixture was concentrated under reduced pressure to give the product (0.5 mL solution) at 20° C. (R)-8-(2-fluoro-4-((1S,2S)-6-hydroxy-2-phenyl-1,2,3,4-tetrahydronaphthalen-1-yl)-5-methoxyphenyl)-1-oxa-8-azaspiro[4.5]decane-3-carbaldehyde was obtained as a yellow solution, m/e+1=516.3

### Step 2: Synthesis of (3S)-3-[1′-[[(3S)-8-[2-fluoro-4-[(1S,2S)-6-hydroxy-2-phenyl-tetralin-1-yl]-5-methoxy-phenyl]-1-oxa-8-azaspiro[4.5]decan-3-yl]methyl]-5-methyl-6-oxo-spiro[2,8-dihydrofuro[2,3-e]isoindole-3,4′-piperidine]-7-yl]piperidine-2,6-dione

To the suspension of(S)-3-(5-methyl-6-oxo-6,8-dihydro-2H,7H-spiro[furo[2,3-e]isoindole-3,4′-piperidin]-7-yl)piperidine-2,6-dione (38.1 mg, 84.5 μmol, 1.05 eq, HCl) in DMF (1 mL) was added KOAc (8.29 mg, 84.5 μmol, 1.05 eq) in portions at 20° C., and stirred for 10 minutes. The mixture was cooled to 0° C., NaBH (OAc) 3 (25,6 mg, 121 μmol, 1.50 eq) and the solution of (R)-8-(2-fluoro-4-((1S,2S)-6-hydroxy-2-phenyl-1,2,3,4-tetrahydronaphthalen-1-yl)-5-methoxyphenyl)-1-oxa-8-azaspiro[4.5]decane-3-carbaldehyde (41.5 mg, 80.5 μmol, 1.00 eq) in formic acid (0.5 mL) was added in portions at 0° C. The reaction mixture was stirred at 0° C. for 1 hour and turned brown solution. The reaction mixture was purified by prep-HPLC(column: Phenomenex Luna C18 200*40 mm*10 μm; mobile phase: [water (FA)-ACN]; B %: 25%-60%, 8 min). (3S)-3-[1′-[[(3S)-8-[2-fluoro-4-[(1S,2S)-6-hydroxy-2-phenyl-tetralin-1-yl]-5-methoxy-phenyl]-1-oxa-8-azaspiro[4.5]decan-3-yl]methyl]-5-methyl-6-oxo-spiro[2,8-dihydrofuro[2,3-e]isoindole-3,4′-piperidine]-7-yl]piperidine-2,6-dione (35.46 mg, 40.80 μmol, 50.70% yield. 100% purity) was obtained as a white solid, which was indicated by HNMR, ¹H NMR (400 MHZ, DMSO-d₆) δ 10.95 (s, 1H), 8.98-9.26 (m, 1H), 6.96-7.21 (m, 3H), 6.75 (br d, J=6.32 Hz, 2H), 6.44-6.61 (m, 3H), 6.13-6.25 (m, 2H), 5.03 (br dd, J=13.35, 5.01 Hz, 1H), 4.67 (br d, J=5.01 Hz, 1H), 4.43-4.52 (m, 2H), 4.29 (br d, J=17.17 Hz, 1H), 4.14 (br d, J=16.93 Hz, 1H), 3.87 (br t, J=7.81 Hz, 1H), 3.47 (br s, 1H), 2.81-3.02 (m, 13H), 2.56-2.67 (m, 5H), 2.38-2.43 (m, 1H), 2.32 (br d, J=6.44 Hz, 2H), 2.15 (br dd, J=12.58, 6.02 Hz, 1H), 1.82-2.04 (m, 6H), 1.58-1.78 (m, 7H), 1.31-1.39 (m, 1H), m/e+1=869.4.

### Compound A227: (S)-3-(1′-(((S)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-5-methyl-6-oxo-6,8-dihydro-2H,7H-spiro[furo[2,3-e]isoindole-3,4′-piperidin]-7-yl)piperidine-2,6-dione

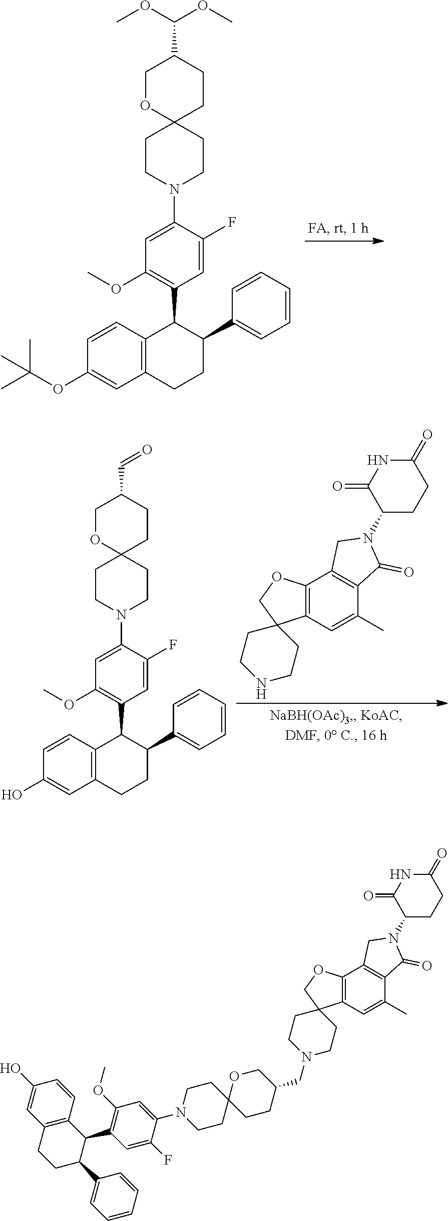

### Step 1: (R)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecane-3-carbaldehyde

To a mixture of(S)-9-(4-((1S,2S)-6-(tert-butoxy)-2-phenyl-1,2,3,4-tetrahydronaphthalen-1-yl)-2-fluoro-5-methoxyphenyl)-3-(dimethoxymethyl)-1-oxa-9-azaspiro[5.5]undecane (20.0 mg. 1.0 Eq, 31.7 μmol) in HCOOH (1.50 mL) was stirred at rt for 1 h. The mixture was concentrated to give (S)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecane-3-carbaldehyde (20.0 mg) crude as yellow oil.

LC-MS (ESI, m/z): mass calcd.529.26; found, 530.3 [M+H]⁺. Step 2: (S)-3-(1′-(((S)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-5-methyl-6-oxo-6,8-dihydro-2H,7H-spiro[furo[2,3-e]isoindole-3,4′-piperidin]-7-yl)piperidine-2,6-dione

To a mixture of(S)-3-(5-methyl-6-oxo-6,8-dihydro-2H,7H-spiro[furo[2,3-e]isoindole-3,4′-piperidin]-7-yl)piperidine-2,6-dione HCl salt (20 mg, 0.049 mmol, 1.3 eq) in DMF (1.50 mL) was added potassium acetate (3.89 mg, 0.040 mmol, 1.05 eq) at rt, then followed by the addition of Sodium triacetoxyborohydride (12 mg, 0.057 mmol, 1.5 eq) and(S)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecane-3-carbaldehyde (20.0 mg, 0.038 mmol, 1.0 eq) in HCOOH (0.30 mL) successively at 0° C. The resulting mixture was stirred at rt for 16 hours, concentrated. The residue was purified by reverse-phase chromatography (0-50% Acetonitrile/0.05% Formic acid) to afford (S) -3-(1′-(((R)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-5-methyl-6-oxo-6,8-dihydro-2H,7H-spiro[furo[2,3-e]isoindole-3,4′-piperidin]-7-yl)piperidine-2,6-dione (4.57 mg, yield: 13.3%) as a white solid.

LC-MS (ESI, m/z): mass calcd. For C₅₃H₅₉FN407. 882.44; found, 883.1 [M+H]⁺.

¹H NMR (400 MHZ, DMSO-d₆): δ 10.96 (s, 1H), 9.13 (s, 1H), 7.06-7.16 (m, 4H), 6.73-6.79 (m, 2H), 6.51-6.61 (m, 2H), 6.47 (dd, J=8.4 Hz, 2.0 Hz, 1H), 6.19 (dd, J=10.0 Hz, 2.0 Hz, 2H), 5.00-5.08 (m, 1H), 4.65-4.69 (m, 1H), 4.43-4.51 (m, 2H), 4.29 (d, J=16.8 Hz, 1H), 4.14 (d, J=16.8 Hz, 1H), 3.65-3.72 (m, 1H), 3.20-3,28 (m, 3H), 2.74-3.03 (m, 13H), 2.59-2.63 (m, 1H), 2.27-2.45 (m, 2H), 2.05-2.22 (m, 4H), 1.83-2.03 (m, 5H), 1.53-1.81 (m, 8H), 1.43-1.52 (m, 1H), 1.29-1.41 (m, 2H).

### Compound A231: (S)-3-(5-chloro-1′-(((S)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione

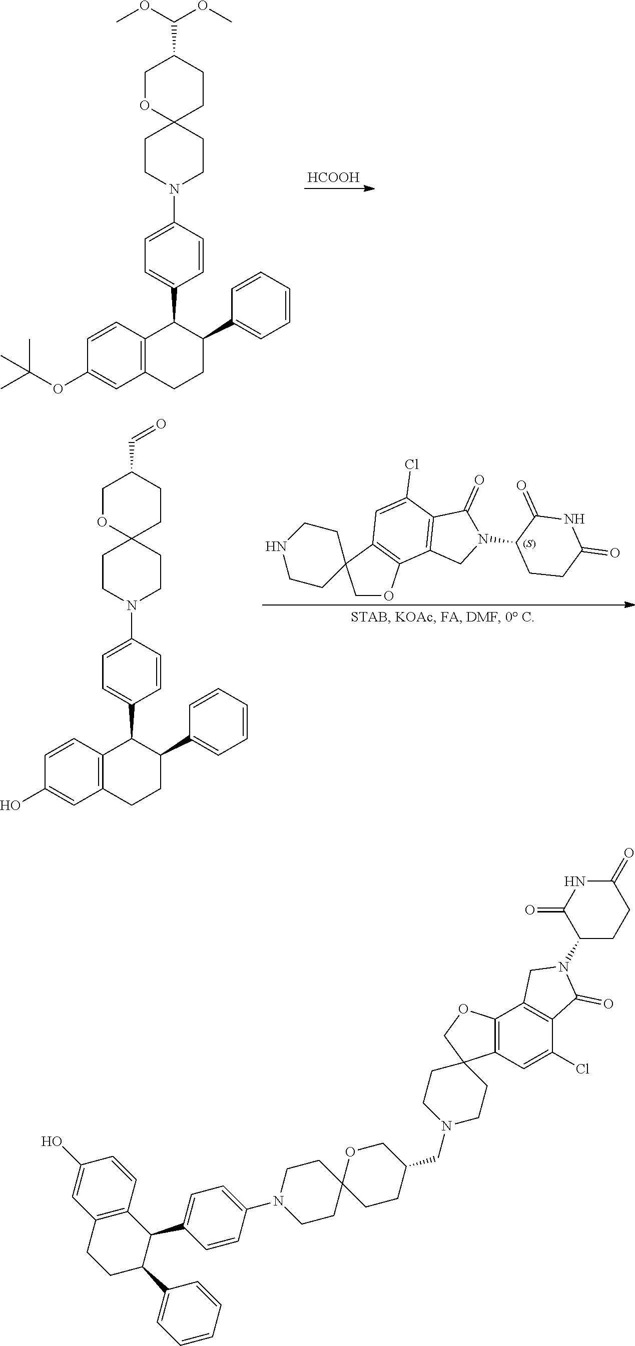

The synthesis of(S)-3-(5-chloro-1′-(((S)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione was similar to Compound A227 and finally to give(S)-3-(5-chloro-1′-(((R)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione (14.71 mg, 40%) as a white solid.

LC-MS (ESI, m/z): mass calcd. For C₅₁H₅₅ClN₄O₆, 854.38; found, 855.4 [M+H]⁺. ¹H NMR (400 MHZ, DMSO-d₆): δ 10.98 (s, 1H), 9.11 (s, 1H), 7.43 (s, 1H), 7.16-7.11 (m, 3H), 6.83 (d, J=6.4 Hz, 2H), 6.66-6.60 (m, 2H), 6.54-6.47 (m, 3H), 6.21 (d, J=8.4 Hz, 2H), 5.07-5.03 (m, 1H), 4.54 (s, 2H), 4.36-4.16 (m, 2H), 4.13-4.12 (m, 1H), 3.68-3.65 (m, 2H), 3.17 (s, 6H), 3.01-2,75 (m, 6H), 2.19-2.13 (m, 2H), 2.07-1.90 (m, 6H), 1.72-1.62 (m, 5H), 1.59-1.52 (m, 3H), 1.34-1.24 (m, 4H).

### Compound A232: (S)-3-(5-chloro-1′-(((S)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione

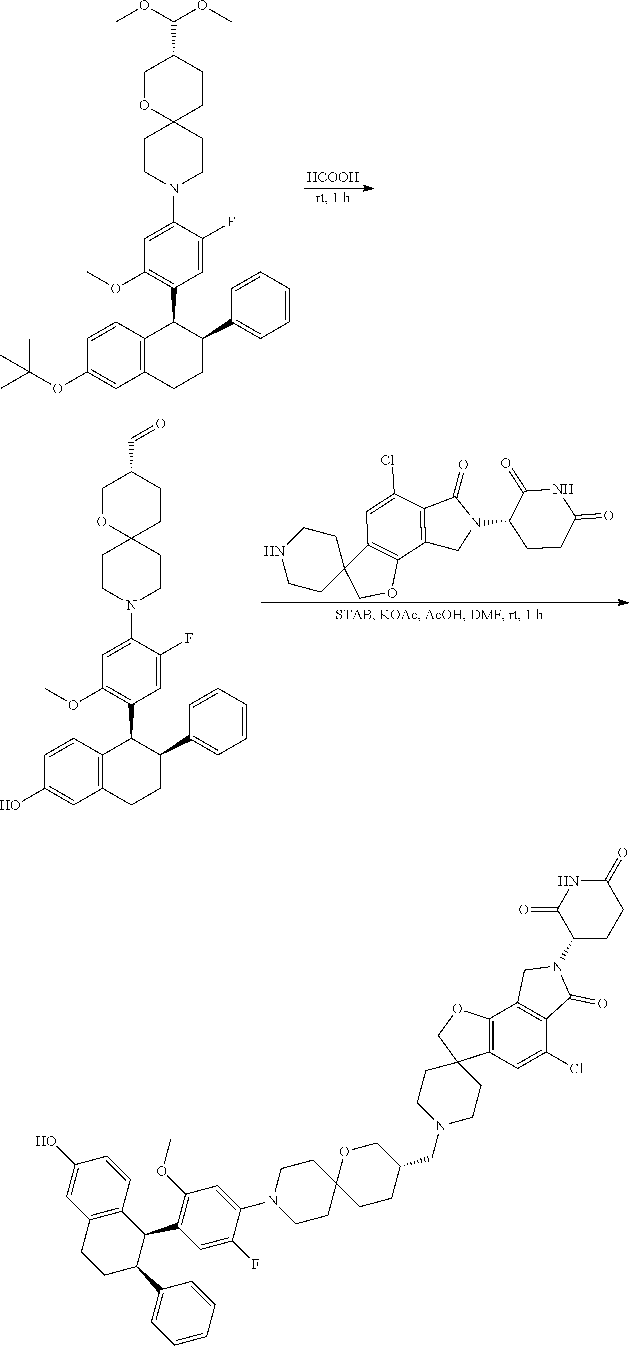

The synthesis of(S)-3-(5-chloro-1′-(((S)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione was similar to Compound A227 and finally to give(S)-3-(5-chloro-1′-(((S)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione (21.7 mg, 43.0%) as a white solid.

LC-MS (ESI, m/2); mass calcd. For C₅₁H₅₅ClN₄O₆, 854.4; found, 855.2 [M+H]⁺.

¹H NMR (400 MHZ, DMSO-d₆) δ 10.98 (s, 1H), 9.10 (s, 1H), 7.43 (s, 1H), 7.20-7.06 (m. 3H), 6.83 (d, J=6.4 Hz, 2H), 6.67-6.58 (m, 2H), 6.57-6.43 (m, 3H), 6.20 (d, J=8.4 Hz, 2H), 5.05 (dd, J=13.2, 5.2 Hz, 1H), 4.59-4.48 (m, 2H), 4.37-4,28 (m, 1H), 4.23-4.10 (m, 2H), 3.70-3.63 (m, 1H), 3.17-3.10 (m, 2H), 3.03-2,74 (m, 8H), 2.63-2.55 (m, 2H), 2.43-2.34 (m, 1H), 2.22-2.08 (m, 3H), 2.04-1.86 (m, 6H), 1.76-1.52 (m, 8H), 1.44-1.28 (m, 3H).

### Compound A233: (S)-3-(5-chloro-1′-(((R)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione

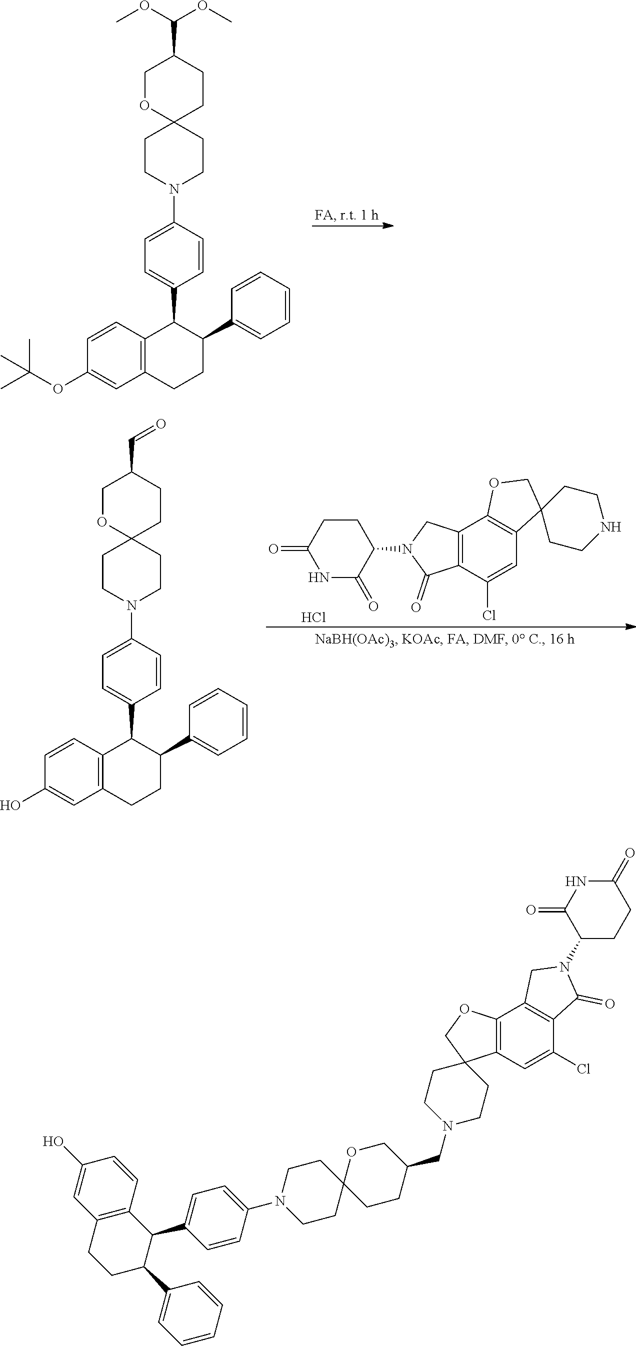

The synthesis of(S)-3-(5-chloro-1′-(((R)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione was similar to Compound A227 and finally to give(S)-3-(5-chloro-1′-(((S)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione (28.42 mg, 37.9%) as a white solid.

LC-MS (ESI, m/z): mass calcd. For C₅₁H₅₅ClN₄O₆, 854.38; found, 855.2 [M+H]⁺.

¹H NMR (400 MHZ, DMSO-d₆): δ 10.98 (s, 1H), 9.09 (s, 1H), 7.43 (s, 1H), 7.08-7.18 (m, 3H), 6.79-6.86 (m, 2H), 6.65 (d, J=8.8 Hz, 1H), 6.60 (d, J=2.4 Hz, 1H), 6.53 (d, J=8.8 Hz, 2H), 6.48 (dd, J=8.4 Hz, 2.4 Hz, 1H), 6.20 (d, J=8.8 Hz, 2H), 5.01-5.09 (m, 1H), 4.50-4.58 (m, 2H), 4.34 (d, J=17.6 Hz, 1H), 4.17 (d, J=17.2 Hz, 1H), 4.12 (d, J=5.2 Hz, 1H), 3.36-3.71 (m, 1H), 3.20-3,27 (m, 1H), 3.09-3.19 (m, 2H), 2.83-3.03 (m, 5H), 2.73-2.83 (m, 2H), 2.55-2.62 (m, 2H), 2.31-2.44 (m, 2H), 1.86-2.24 (m, 9H), 1.28-1.78 (m, 10H).

### Compound A234: (S)-3-(5-chloro-1′-(((R)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione

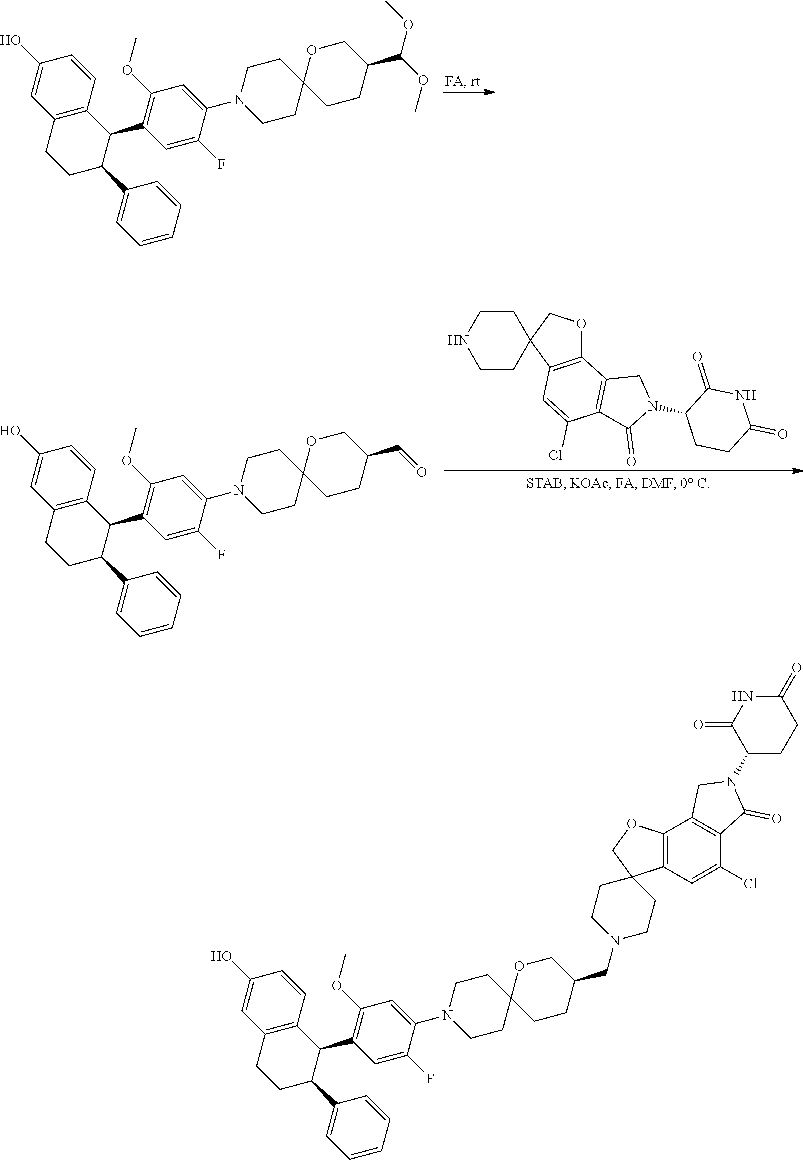

The synthesis of(S)-3-(5-chloro-1′-(((R)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione was similar to Compound A227 and finally to give(S)-3-(5-chloro-1′-(((S)-9-(2-fluoro-4-((1S,2S)-6-hydroxy-2-phenyl-1,2,3,4-tetrahydronaphthalen-1-yl)-5-methoxyphenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione (9.54 mg, 25.3%) as a white solid.

LC-MS (ESI, m/z): mass calcd. For C₅₂H₅₆ClFN407, 902.38; found, 903.2 [M+H]⁺.

¹H NMR (400 MHZ, DMSO-d₆): δ 10.98 (s, 1H), 8.19 (s, 1H), 7.46-7.37 (m, 1H), 7.14-7.03 (m, 3H), 6.80-6.62 (m, 2H), 6.62-6.52 (m, 2H), 6.51-6.45 (m, 1H), 6.27-6.15 (m, 2H), 5.05 (dd, J=13.2 Hz, 5.2 Hz, 1H), 4.67 (d, J=5.2 Hz, 1H), 4.58-4.49 (m, 2H), 4.40-4.30 (m, 1H), 4.25-4.15 (m, 1H), 3.71-3.66 (m, 1H), 3.28-3,23 (m, 2H), 3.02-2.83 (m, 11H), 2.81-2,72 (m, 2H), 2.64-2.57 (m, 1H), 2.45-2.36 (m, 1H), 2.22-2.10 (m, 3H), 2.01-1.87 (m, 5H), 1.77-1.55 (m, 8H), 1.50-1.29 (m, 3H).

### Compound A235: (S)-3-(1′-(((R)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-5-methoxy-6-oxo-6,8-dihydro-2H,7H-spiro[furo[2,3-e]isoindole-3,4′-piperidin]-7-yl)piperidine-2,6-dione

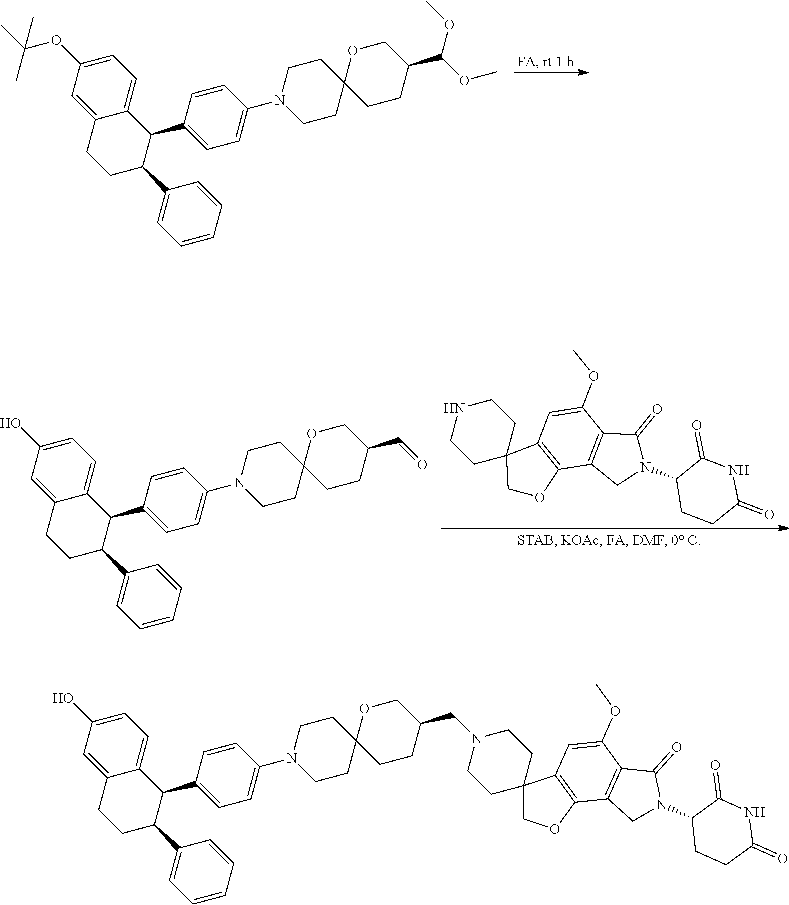

The synthesis of(S)-3-(1′-(((R)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-5-methoxy-6-oxo-6,8-dihydro-2H,7H-spiro[furo[2,3-e]isoindole-3,4′-piperidin]-7-yl)piperidine-2,6-dione was similar to Compound A227 and finally to give(S)-3-(1′-(((S)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-5-methoxy-6-oxo-6,8-dihydro-2H,7H-spiro[furo[2,3-e]isoindole-3,4′-piperidin]-7-yl)piperidine-2,6-dione (17.43 mg, 49.8%) as a white solid.

LC-MS (ESI, m/z): mass calcd. For C₅₂H₅₈N₄O₇, 850.43; found, 851.7 [M+H]⁺.

¹H NMR (400 MHZ, DMSO-d₆): δ 10.92 (s, 1H), 8.31 (s, 1H), 7.19-7.08 (m, 3H), 7.01 (s, 1H), 6.83 (d. J=6.8 Hz, 2H), 6.68-6.59 (m, 2H), 6.56-6.43 (m, 3H), 6.20 (d. J=8.4 Hz, 2H), 4.99 (dd, J=13.2 Hz, 4.8 Hz, 1H), 4.52-4.40 (m, 2H), 4.31-4,20 (m, 1H), 4.15-4.06 (m, 2H), 3.82 (s, 3H), 3.69-3.65 (m, 1H), 3.31-3,22 (m, 3H), 3.16-3.10 (m, 2H), 2.97-2.83 (m, 5H), 2.81-2,71 (m, 2H), 2.64-2.55 (m, 1H), 2.40-2.31 (m, 1H), 2.20-2.12 (m, 2H), 2.04-1.87 (m, 6H), 1.75-1.50 (m, 8H), 1.45-1.37 (m, 1H), 1.37-1.24 (m, 2H).

### Compound A236: (S)-3-(1′-(((S)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-5-methoxy-6-oxo-6,8-dihydro-2H,7H-spiro[furo[2,3-e]isoindole-3,4′-piperidin]-7-yl)piperidine-2,6-dione

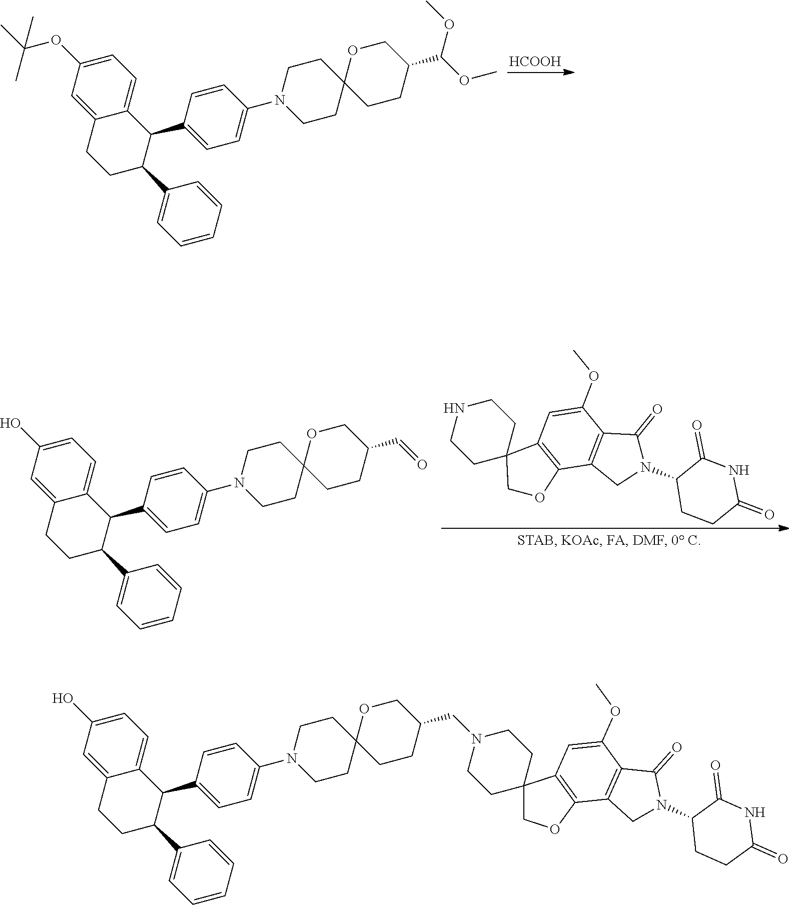

The synthesis of(S)-3-(1′-(((S)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-5-methoxy-6-oxo-6,8-dihydro-2H,7H-spiro[furo[2,3-c]isoindole-3,4′-piperidine]-7-yl)piperidinic-2,6-dione was similar to Compound A227 and finally to give(S)-3-(1′-(((R)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-5-methoxy-6-oxo-6,8-dihydro-2H,7H-spiro[furo[2,3-e]isoindole-3,4′-piperidin]-7-yl)piperidine-2,6-dione (20.81 mg, 58.9%) as a white solid.

LC-MS (ESI, m/z): mass calcd. For C₅₂H₅₈N₄O₇, 850.43; found, 851.7 [M+H]⁺.

¹H NMR (400 MHZ, DMSO-d₆): δ 10.92 (s, 1H), 8.25 (s, 1H), 7.18-7.08 (m, 3H), 7.01 (s, 1H), 6.83 (d, J=6.8 Hz, 2H), 6.73-6.58 (m, 2H), 6.56-6.43 (m, 3H), 6.20 (d, J=8.4 Hz, 2H), 4.99 (dd, J=13.2 Hz, 5.2 Hz, 1H), 4.50-4.38 (m, 2H), 4.32-4,23 (m, 1H), 4.14-4.07 (m, 2H), 3.82 (s, 3H), 3.66-3.65 (m, 1H), 3.30-3,21 (m, 3H), 3.17-3.11 (m, 2H), 2.98-2.83 (m, 5H), 2.80-2,73 (m, 2H), 2.62-2.54 (m, 1H), 2.39-2.31 (m, 1H), 2.20-2.13 (m, 2H), 2.05-1.87 (m, 6H), 1.77-1.51 (m, 8H), 1.45-1.38 (m, 1H), 1.36-1.26 (m, 2H).

The rest of examples were prepared in a manner analogous to Compound A208 by reductive amination using same Method.

The following examples were prepared in a manner analogous to compound A8 or A208 by reductive amination.

TABLE 4
Characterization Data for “A” Compounds

| Compound | | Calcd. | Found | LCMS |
|---|---|---|---|---|
| No. | ¹H NMR | (M + H)⁺ | (M + H)⁺ | method |
| A1 | ¹H NMR (400 M Hz, DMSO-d₆) δ 10.87 (s, | 781.4 | 781.4 | F |
| | 1H), 8.61 (d, J = 8.2 Hz, 1H), 8.21 (s, 1H), | | | |
| | 7.59 (d, J = 8.0 Hz, 1H), 7.26 (d, J = 8.0 Hz, | | | |
| | 1H), 7.17-7.09 (m, 3H), 6.83 (d, J = 6.6 Hz, | | | |
| | 2H), 6.66-6.58 (m, 2H), 6.55-6.45 (m, | | | |
| | 3H), 6.20 (d, J = 8.6 Hz, 2H), 4.78-4.68 (m, | | | |
| | 1H), 4.12 (d, J = 4.8 Hz, 1H), 4.01 (s, 3H), | | | |
| | 3.53-3.23 (m, 6H), 3.10 (s, 4H), 2.97-2.75 | | | |
| | (m, 4H), 2.57-2.52 (m, 2H), 2.27-1.98 (m, | | | |
| | 6H), 1.78-1.61 (m, 4H), 1.21-1.09 (m, 2H) | | | |
| A2 | ¹H NMR (400 MHz, DMSO) δ 10.83 (s, 1H), | 741.4 | 741.4 | F |
| | 8.49 (d, J = 8.4 Hz, 1H), 7.56 (d, J = 8.0 Hz, | | | |
| | 1H), 7.30 (d, J = 8.4 Hz, 1H), 7.20-7.06 (m, | | | |
| | 3H), 6.83 (d, J = 6.4 Hz, 2H), 6.69-6.44 (m, | | | |
| | 5H), 6.20 (d, J = 8.4 Hz, 2H), 4.76-4.66 (m, | | | |
| | 1H), 4.47-4.39 (m, 1H), 4.16-4.02 (m, | | | |
| | 2H), 3.77 (d, J = 11.2 Hz, 1H), 3.56-3.18 | | | |
| | (m, 8H), 3.02-2.88 (m, 4H), 2.82-2.70 (m, | | | |
| | 2H), 2.22-1.94 (m, 6H), 1.81-1.61 (m, | | | |
| | 5H), 1.24-1.08 (m, 2H). | | | |
| A3 | ¹H NMR (400 MHz, DMSO) δ 10.86 (s, 1H), | 712.4 | 712.4 | F |
| | 9.01 (d, J = 8.4 Hz, 1H), 8.57 (d, J = 1.8 Hz, | | | |
| | 1H), 8.27 (s, 1H), 7.98 (d, J = 8.2 Hz, 1H), | | | |
| | 7.93-7.84 (m, 1H), 7.19-7.06 (m, 3H), | | | |
| | 6.83 (d, J = 6.6 Hz, 2H), 6.67-6.44 (m, 5H), | | | |
| | 6.20 (d, J = 8.6 Hz, 2H), 4.83-4.72 (m, 1H), | | | |
| | 4.13 (d, J = 5.0 Hz, 1H), 3.51 (d, J = 9.8 Hz, | | | |
| | 2H), 3.30-3.27 (m, 2H), 3.03-2.88 (m, | | | |
| | 4H), 2.85-2.75 (m, 1H), 2.70-2.63 (m, | | | |
| | 1H), 2.57-2.52 (m, 1H), 2.48-2.44 (m, | | | |
| | 1H), 2.26-2.07 (m, 4H), 1.99 (t, J = 10.2 Hz, | | | |
| | 3H), 1.82-1.55 (m, 8H), 1.21-1.07 (m, 2H). | | | |
| A4 | ¹H NMR (400 MHz, DMSO) δ 10.83 (s, 1H), | 741.4 | 741.3 | E |
| | 8.49 (d, J = 8.2 Hz, 1H), 7.56 (d, J = 8.2 Hz, | | | |
| | 1H), 7.30 (d, J = 8.4 Hz, 1H), 7.20-7.07 (m, | | | |
| | 3H), 6.83 (d, J = 6.6 Hz, 2H), 6.66-6.44 (m, | | | |
| | 5H), 6.20 (d, J = 8.6 Hz, 2H), 4.75-4.67 (m, | | | |
| | 1H), 4.48-4.38 (m, 1H), 4.16-4.03 (m, | | | |
| | 2H), 3.77 (d, J = 12.0 Hz, 1H), 3.51 (d, J = | | | |
| | 9.6 Hz, 2H), 3.31-3.24 (m, 2H), 3.23-3.15 | | | |
| | (m, 2H), 3.01-2.87 (m, 4H), 2.83-2.70 (m, | | | |
| | 2H), 2.56-2.52 (m, 1H), 2.48-2.44 (m, | | | |
| | 1H), 2.25-2.04 (m, 5H), 2.02-1.93 (m, | | | |
| | 1H), 1.81-1.58 (m, 5H), 1.22-1.07 (m, 2H). | | | |
| A5 | ¹H NMR (400 MHz, DMSO) δ 10.85 (s, 1H), | 743.9 | 743.9 | E |
| | 8.86 (d, J = 8.4 Hz, 1H), 8.31 (s, 1H), 8.06 (s, | | | |
| | 1H), 7.58 (s, 1H), 7.21-7.05 (m, 3H), 6.83 | | | |
| | (d, J = 6.6 Hz, 2H), 6.68-6.44 (m, 5H), 6.20 | | | |
| | (d, J = 8.6 Hz, 2H), 4.81-4.69 (m, 1H), 4.12 | | | |
| | (d, J = 5.0 Hz, 1H), 3.93 (s, 3H), 3.51 (d, J = | | | |
| | 12.4 Hz, 4H), 3.32-3.24 (m, 2H), 3.12 (s, | | | |
| | 4H), 3.06-2.86 (m, 3H), 2.85-2.74 (m, | | | |
| | 1H), 2.60-2.53 (m, 1H), 2.47-2.41 (m, | | | |
| | 1H), 2.36-1.93 (m, 6H), 1.80-1.54 (m, | | | |
| | 4H), 1.23-1.07 (m, 2H). | | | |
| A6 | ¹H NMR (400 MHz, DMSO) δ 10.84 (s, 1H), | 741.9 | 741.9 | C |
| | 8.50 (d, J = 8.2 Hz, 1H), 7.57 (d, J = 8.2 Hz, | | | |
| | 1H), 7.30 (d, J = 8.4 Hz, 1H), 7.24-7.06 (m, | | | |
| | 3H), 6.83 (d, J = 6.4 Hz, 2H), 6.72-6.57 (m, | | | |
| | 2H), 6.56-6.40 (m, 3H), 6.21 (d, J = 8.6 Hz, | | | |
| | 2H), 4.79-4.65 (m, 1H), 4.51-4.40 (m, | | | |
| | 1H), 4.20-4.03 (m, 2H), 3.77 (d, J = 11.4 | | | |
| | Hz, 2H), 3.35-3.12 (m, 5H), 3.05-2.80 (m, | | | |
| | 5H), 2.80-2.66 (m, 2H), 2.48-2.36 (m, | | | |
| | 1H), 2.24-2.03 (m, 5H), 2.03-1.94 (m, | | | |
| | 1H), 1.81-1.59 (m, 5H), 1.25-1.07 (m, 2H). | | | |
| A7 | ¹H NMR (400 MHz, DMSO) δ 10.93 (s, 1H), | 780.9 | 780.9 | C |
| | 8.37 (s, 1H), 7.17-7.08 (m, 3H), 7.00 (t, J = | | | |
| | 8.1 Hz, 2H), 6.79 (dd, J = 19.5, 7.5 Hz, 2H), | | | |
| | 6.65-6.55 (m, 2H), 6.47 (d, J = 10.1 Hz, | | | |
| | 1H), 6.30 (d, J = 8.2 Hz, 1H), 6.17 (d, J = 8.4 | | | |
| | Hz, 1H), 6.05 (d, J = 8.4 Hz, 1H), 5.03 (dd, | | | |
| | J = 12.9, 4.9 Hz, 1H), 4.35 (d, J = 9.9 Hz, 1H), | | | |
| | 4.26 (d, J = 16.7 Hz, 1H), 4.15-4.05 (m, | | | |
| | 2H), 4.00-3.92 (m, 1H), 3.91-3.78 (m, | | | |
| | 3H), 3.72 (d, J = 7.1 Hz, 1H), 3.69-3.59 (m, | | | |
| | 2H), 3.57-3.47 (m, 3H), 3.21-3.14 (m, | | | |
| | 2H), 3.06-2.87 (m, 5H), 2.76 (dd, J = 32.0, | | | |
| | 8.0 Hz, 2H), 2.69-2.58 (m, 2H), 2.39-2.24 | | | |
| | (m, 4H), 2.19-2.04 (m, 2H), 1.98-1.86 (m, | | | |
| | 2H), 1.75-1.68 (m, 1H). | | | |
| A8 | ¹H NMR (400 MHz, DMSO) δ 10.94 (s, 1H), | 780.9 | 780.9 | C |
| | 9.12 (s, 1H), 7.19-7.07 (m, 4H), 7.04-6.95 | | | |
| | (m, 2H), 6.80 (dd, J = 16.7, 7.5 Hz, 2H), 6.66- | | | |
| | 6.57 (m, 2H), 6.47 (dd, J = 8.3, 2.6 Hz, 1H), | | | |
| | 6.31 (d, J = 8.3 Hz, 1H), 6.18 (d, J = 8.4 Hz, | | | |
| | 1H), 6.06 (d, J = 8.3 Hz, 1H), 5.03 (dd, J = | | | |
| | 13.1, 4.9 Hz, 1H), 4.31 (d, J = 9.6 Hz, 1H), | | | |
| | 4.21 (d, J = 22.7 Hz, 2H), 4.15-4.10 (m, | | | |
| | 1H), 4.00-3.87 (m, 3H), 3.87-3.71 (m, | | | |
| | 3H), 3.70-3.60 (m, 2H), 3.59-3.49 (m, | | | |
| | 3H), 3.03-2.76 (m, 6H), 2.71-2.58 (m, | | | |
| | 3H), 2.44-2.29 (m, 3H), 2.09 (dd, J = 14.0, | | | |
| | 8.4 Hz, 1H), 2.01-1.82 (m, 3H), 1.69 (d, J = | | | |
| | 11.3 Hz, 1H). | | | |
| A9 | ¹H NMR (400 MHz, DMSO) δ 10.93 (s, 1H), | 823.0 | 823.1 | E |
| | 8.34 (s, 1H), 7.17 (d, J = 8.2 Hz, 1H), 7.09- | | | |
| | 6.94 (m, 4H), 6.78-6.68 (m, 2H), 6.59- | | | |
| | 6.49 (m, 2H), 6.46-6.41 (m, 1H), 6.39- | | | |
| | 6.25 (m, 2H), 6.10 (s, 1H), 5.12-4.96 (m, | | | |
| | 1H), 4.64 (d, J = 5.0 Hz, 1H), 4.43-4.23 (m, | | | |
| | 2H), 4.15-4.05 (m, 1H), 4.00-3.90 (m, | | | |
| | 1H), 3.80 (d, J = 11.2 Hz, 1H), 3.25-3.10 | | | |
| | (m, 3H), 3.00 (s, 2H), 2.94-2.82 (m, 10H), | | | |
| | 2.72 (t, J = 10.6 Hz, 1H), 2.55 (s, 1H), 2.48- | | | |
| | 2.34 (m, 4H), 2.22-2.05 (m, 2H), 2.01- | | | |
| | 1.91 (m, 3H), 1.73 (t, J = 10.2 Hz, 1H), 1.68- | | | |
| | 1.50 (m, 5H), 1.44-1.34 (m, 2H). | | | |
| A10 | ¹H NMR (400 MHz, DMSO) δ 10.93 (s, 1H), | 823.0 | 823.1 | C |
| | 8.28 (s, 1H), 7.17 (d, J = 8.2 Hz, 1H), 7.11- | | | |
| | 6.94 (m, 4H), 6.76-6.67 (m, 2H), 6.59- | | | |
| | 6.51 (m, 2H), 6.48-6.40 (m, 1H), 6.39- | | | |
| | 6.23 (m, 2H), 6.10 (s, 1H), 5.08-4.94 (m, | | | |
| | 1H), 4.64 (d, J = 5.2 Hz, 1H), 4.37-4.19 (m, | | | |
| | 2H), 4.16-4.04 (m, 1H), 4.02-3.92 (m, | | | |
| | 1H), 3.85-3.78 (m, 1H), 3.27-3.11 (m, | | | |
| | 3H), 3.00 (s, 2H), 2.95-2.82 (m, 11H), 2.76 | | | |
| | -2.65 (m, 1H), 2.63-2.54 (m, 1H), 2.48- | | | |
| | 2.34 (m, 4H), 2.21-2.06 (m, 2H), 2.01- | | | |
| | 1.88 (m, 3H), 1.82-1.69 (m, 1H), 1.67- | | | |
| | 1.50 (m, 5H), 1.46-1.36 (m, 2H). | | | |
| A11 | ¹H NMR (400 MHz, DMSO) δ 10.93 (s, 1H), | 823.0 | 823.1 | C |
| | 8.23 (s, 1H), 7.05 (t, J = 7.8 Hz, 4H), 6.93 (s, | | | |
| | 1H), 6.72 (d, J = 5.8 Hz, 2H), 6.55 (d, J = | | | |
| | 10.8 Hz, 2H), 6.48-6.41 (m, 1H), 6.38- | | | |
| | 6.25 (m, 2H), 6.10 (s, 1H), 5.11-4.94 (m, | | | |
| | 1H), 4.64 (d, J = 5.0 Hz, 1H), 4.28-4.10 (m, | | | |
| | 3H), 3.94-3.76 (m, 3H), 3.26-3.10 (m, | | | |
| | 3H), 3.00 (s, 2H), 2.96-2.85 (m, 10H), 2.79- | | | |
| | 2.72 (m, 1H), 2.62-2.54 (m, 1H), 2.45- | | | |
| | 2.36 (m, 3H), 2.24-2.08 (m, 2H), 1.98- | | | |
| | 1.88 (m, 3H), 1.76-1.66 (m, 1H), 1.65- | | | |
| | 1.44 (m, 5H), 1.47-1.36 (m, 2H). | | | |
| A12 | ¹H NMR (400 MHz, DMSO) δ 10.93 (s, 1H), | 823.0 | 823.1 | C |
| | 8.32 (s, 2H), 7.09-6.98 (m, 4H), 6.93 (s, | | | |
| | 1H), 6.77-6.67 (m, 2H), 6.61-6.50 (m, | | | |
| | 2H), 6.47-6.40 (m, 1H), 6.36-6.21 (m, | | | |
| | 2H), 6.10 (s, 1H), 5.09-4.97 (m, 1H), 4.64 | | | |
| | (d, J = 5.0 Hz, 1H), 4.32-4.09 (m, 4H), 3.94- | | | |
| | 3.74 (m, 3H), 3.24-3.12 (m, 2H), 3.00 (s, | | | |
| | 2H), 2.94-2.82 (m, 10H), 2.78-2.67 (m, | | | |
| | 1H), 2.63-2.52 (m, 2H), 2.45-2.33 (m, | | | |
| | 3H), 2.20-2.06 (m, 2H), 1.95 (d, J = 5.2 Hz, | | | |
| | 3H), 1.66-1.51 (m, 5H), 1.46-1.36 (m, 2H). | | | |
| A13 | ¹H NMR (400 MHz, DMSO) δ 10.82 (s, 1H), | 797.9 | 797.8 | F |
| | 8.49 (d, J = 8.0 Hz, 1H), 7.56 (d, J = 8.0 Hz, | | | |
| | 1H), 7.30 (d, J = 8.4 Hz, 1H), 7.19-7.05 (m, | | | |
| | 3H), 6.82 (d, J = 6.8 Hz, 2H), 6.67-6.44 (m, | | | |
| | 5H), 6.20 (d, J = 8.0 Hz, 2H), 4.76-4.66 (m, | | | |
| | 1H), 4.43 (d, J = 10.8 Hz, 1H), 4.15-4.02 | | | |
| | (m, 2H), 3.87 (t, J = 7.4 Hz, 1H), 3.77 (d, J = | | | |
| | 11.2 Hz, 1H), 3.44-3.43 (m, 1H), 3.30- | | | |
| | 3.26 (m, 1H), 3.22-3.17 (m, 1H), 3.08- | | | |
| | 2.88 (m, 9H), 2.82-2.72 (m, 2H), 2.65- | | | |
| | 2.54 (m, 2H), 2.35 (d, J = 6.4 Hz, 2H), 2.17- | | | |
| | 1.91 (m, 5H), 1.77-1.53 (m, 6H), 1.38- | | | |
| | 1.28 (m, 1H). | | | |
| A14 | ¹H NMR (400 MHz, DMSO) δ 10.82 (s, 1H), | 797.9 | 797.8 | F |
| | 8.49 (d, J = 8.2 Hz, 1H), 7.56 (d, J = 8.2 Hz, | | | |
| | 1H), 7.29 (d, J = 8.3 Hz, 1H), 7.18-7.06 (m, | | | |
| | 3H), 6.82 (d, J = 6.6 Hz, 2H), 6.67-6.57 (m, | | | |
| | 2H), 6.56-6.45 (m, 3H), 6.20 (d, J = 8.6 Hz, | | | |
| | 2H), 4.76-4.66 (m, 1H), 4.47-4.39 (m, | | | |
| | 1H), 4.14-4.02 (m, 2H), 3.87 (t, J = 7.8 Hz, | | | |
| | 1H), 3.76 (d, J = 11.6 Hz, 1H), 3.43-3.42 | | | |
| | (m, 1H), 3.30-3.27 (m, 1H), 3.21-3.16 (m, | | | |
| | 1H), 3.08-2.90 (m, 9H), 2.82-2.71 (m, | | | |
| | 2H), 2.61-2.53 (m, 2H), 2.41-2.30 (m, | | | |
| | 2H), 2.20-2.05 (m, 3H), 2.03-1.88 (m, | | | |
| | 2H), 1.73-1.55 (m, 6H), 1.37-1.27 (m, 1H). | | | |
| A15 | ¹H NMR (400 MHz, DMSO) δ 10.80 (s, 1H), | 797.9 | 797.8 | E |
| | 8.49 (d, J = 8.4 Hz, 1H), 7.56 (d, J = 8.2 Hz, | | | |
| | 1H), 7.30 (d, J = 8.4 Hz, 1H), 7.18-7.07 (m, | | | |
| | 3H), 6.82 (d, J = 6.6 Hz, 2H), 6.66-6.44 (m, | | | |
| | 5H), 6.20 (d, J = 8.6 Hz, 2H), 4.76-4.66 (m, | | | |
| | 1H), 4.46-4.39 (m, 1H), 4.16-4.02 (m, | | | |
| | 2H), 3.87 (t, J = 7.8 Hz, 1H), 3.77 (d, J = 11.6 | | | |
| | Hz, 1H), 3.45-3.43 (m, 1H), 3.27-3.21 (m, | | | |
| | 2H), 3.10-2.85 (m, 9H), 2.81-2.71 (m, | | | |
| | 2H), 2.68-2.52 (m, 2H), 2.39-2.30 (m, | | | |
| | 2H), 2.19-2.03 (m, 3H), 2.01-1.87 (m, | | | |
| | 2H), 1.75-1.52 (m, 6H), 1.37-1.28 (m, 1H). | | | |
| A16 | ¹H NMR (400 MHz, DMSO) δ 10.83 (s, 1H), | 797.9 | 797.8 | E |
| | 8.49 (d, J = 8.2 Hz, 1H), 7.56 (d, J = 8.2 Hz, | | | |
| | 1H), 7.30 (d, J = 8.4 Hz, 1H), 7.17-7.08 (m, | | | |
| | 3H), 6.82 (d, J = 6.6 Hz, 2H), 6.67-6.57 (m, | | | |
| | 2H), 6.57-6.45 (m, 3H), 6.20 (d, J = 8.6 Hz, | | | |
| | 2H), 4.75-4.66 (m, 1H), 4.48-4.38 (m, | | | |
| | 1H), 4.15-4.01 (m, 2H), 3.87 (t, J = 7.8 Hz, | | | |
| | 1H), 3.77 (d, J = 11.4 Hz, 1H), 3.46-3.44 | | | |
| | (m, 1H), 3.24-3.15 (m, 2H), 3.09-2.89 (m, | | | |
| | 9H), 2.81-2.71 (m, 2H), 2.62-2.52 (m, | | | |
| | 2H), 2.40-2.29 (m, 2H), 2.20-2.03 (m, | | | |
| | 3H), 2.02-1.87 (m, 2H), 1.74-1.54 (m, | | | |
| | 6H), 1.37-1.28 (m, 1H). | | | |
| A17 | ¹H NMR (400 MHz, DMSO) δ 10.92 (s, 1H), | 808.9 | 808.8 | F |
| | 8.26 (s, 1H), 7.18-7.07 (m, 3H), 7.04 (s, | | | |
| | 1H), 6.92 (s, 1H), 6.82 (d, J = 6.6 Hz, 2H), | | | |
| | 6.67-6.44 (m, 5H), 6.20 (d, J = 8.6 Hz, 2H), | | | |
| | 5.06-4.99 (m, 1H), 4.31-4.23 (m, 2H), 4.17- | | | |
| | 4.08 (m, 2H), 3.93-3.81 (m, 3H), 3.43 (t, J = | | | |
| | 7.8 Hz, 1H), 3.31-3.23 (m, 1H), 3.19- | | | |
| | 3.11 (m, 1H), 3.07-2.85 (m, 9H), 2.78- | | | |
| | 2.70 (m, 1H), 2.62-2.53 (m, 2H), 2.40- | | | |
| | 2.29 (m, 3H), 2.14-2.03 (m, 2H), 1.98- | | | |
| | 1.87 (m, 2H), 1.78-1.53 (m, 6H), 1.33 (m, 1H). | | | |
| A18 | ¹H NMR (400 MHz, DMSO) δ 10.92 (s, 1H), | 808.9 | 808.8 | F |
| | 8.30 (s, 1H), 7.18-7.07 (m, 3H), 7.04 (s, | | | |
| | 1H), 6.93 (s, 1H), 6.82 (d, J = 6.6 Hz, 2H), | | | |
| | 6.68-6.44 (m, 5H), 6.20 (d, J = 8.6 Hz, 2H), | | | |
| | 5.06-4.99 (m, 1H), 4.31-4.23 (m, 2H), 4.17- | | | |
| | 4.09 (m, 2H), 3.93-3.79 (m, 3H), 3.44 (t, J = | | | |
| | 7.8 Hz, 1H), 3.31-3.23 (m, 1H), 3.18- | | | |
| | 3.10 (m, 1H), 3.07-2.87 (m, 9H), 2.79- | | | |
| | 2.70 (m, 1H), 2.61-2.53 (m, 2H), 2.41- | | | |
| | 2.27 (m, 3H), 2.17-2.04 (m, 2H), 1.98- | | | |
| | 1.88 (m, 2H), 1.74-1.52 (m, 6H), 1.36- | | | |
| | 1.28 (m, 1H). | | | |
| A19 | ¹H NMR (400 MHz, DMSO) δ 10.93 (s, 1H), | 808.9 | 808.8 | E |
| | 8.35 (s, 1H), 7.18-7.07 (m, 3H), 7.04 (s, | | | |
| | 1H), 6.93 (s, 1H), 6.82 (d, J = 6.6 Hz, 2H), | | | |
| | 6.67-6.44 (m, 5H), 6.19 (d, J = 8.6 Hz, 2H), | | | |
| | 5.06-4.99 (m, 1H), 4.31-4.22 (m, 2H), 4.17- | | | |
| | 4.08 (m, 2H), 3.94-3.77 (m, 3H), 3.43 (t, J = | | | |
| | 8.0 Hz, 1H), 3.30-3.25 (m, 1H), 3.17- | | | |
| | 3.12 (m, 1H), 3.07-2.85 (m, 9H), 2.79- | | | |
| | 2.69 (m, 1H), 2.62-2.53 (m, 2H), 2.40- | | | |
| | 2.29 (m, 3H), 2.15-2.03 (m, 2H), 1.98- | | | |
| | 1.88 (m, 2H), 1.78-1.50 (m, 6H), 1.37- | | | |
| | 1.28 (m, 1H). | | | |
| A20 | ¹H NMR (400 MHz, DMSO) δ 10.93 (s, 1H), | 808.9 | 808.8 | E |
| | 8.33 (s, 1H), 7.18-7.07 (m, 3H), 7.04 (s, | | | |
| | 1H), 6.93 (s, 1H), 6.82 (d, J = 6.6 Hz, 2H), | | | |
| | 6.67-6.45 (m, 5H), 6.19 (d, J = 8.6 Hz, 2H), | | | |
| | 5.06-4.99 (m, 1H), 4.32-4.22 (m, 2H), 4.16- | | | |
| | 4.08 (m, 2H), 3.94-3.78 (m, 3H), 3.43 (t, J = | | | |
| | 8.0 Hz, 1H), 3.31-3.24 (m, 1H), 3.18- | | | |
| | 3.11 (m, 1H), 3.06-2.86 (m, 9H), 2.79- | | | |
| | 2.71 (m, 1H), 2.62-2.53 (m, 2H), 2.40- | | | |
| | 2.28 (m, 3H), 2.16-2.03 (m, 2H), 1.98- | | | |
| | 1.88 (m, 2H), 1.75-1.51 (m, 6H), 1.37- | | | |
| | 1.28 (m, 1H). | | | |
| A21 | ¹H NMR (400 MHz, MeOD) δ 7.15 (s, 1H), | 782.9 | 782.8 | C |
| | 7.07 (dd, J = 7.4, 4.0 Hz, 4H), 6.80-6.73 (m, | | | |
| | 2H), 6.67-6.61 (m, 2H), 6.57 (d, J = 8.3 Hz, | | | |
| | 1H), 6.51 (dd, J = 8.3, 2.6 Hz, 2H), 6.28 (s, | | | |
| | 1H), 5.51 (s, 1H), 5.10 (dd, J = 13.3, 5.2 Hz, | | | |
| | 1H), 4.81 (d, J = 5.3 Hz, 1H), 4.41-4.27 (m, | | | |
| | 3H), 4.03 (dd, J = 11.0, 7.9 Hz, 2H), 3.62 (d, J = | | | |
| | 9.2 Hz, 2H), 3.46 (dd, J = 17.5, 12.1 Hz, | | | |
| | 2H), 3.26 (d, J = 7.3 Hz, 2H), 3.05 (s, 4H), | | | |
| | 2.94 (ddd, J = 18.3, 15.7, 5.4 Hz, 3H), 2.80 | | | |
| | (dd, J = 11.9, 7.6 Hz, 3H), 2.68 (s, 2H), 2.53- | | | |
| | 2.41 (m, 1H), 2.38-2.25 (m, 2H), 2.16 (dd, J = | | | |
| | 11.2, 6.2 Hz, 1H), 1.95 (t, J = 10.1 Hz, 3H), | | | |
| | 1.68 (dd, J = 15.3, 5.5 Hz, 1H), 1.52-1.42 | | | |
| | (m, 2H). | | | |
| A22 | ¹H NMR (400 MHz, MeOD) δ 8.29 (s, 1H), | 808.0 | 808.0 | C |
| | 7.29 (s, 1H), 7.12-6.87 (m, 4H), 6.74-6.66 | | | |
| | (m, 2H), 6.59 (d, J = 8.4 Hz, 1H), 6.54 (dd, J = | | | |
| | 9.7, 5.5 Hz, 3H), 6.42 (dd, J = 8.3, 2.4 Hz, | | | |
| | 1H), 6.21 (d, J = 8.6 Hz, 2H), 5.02 (dd, J = | | | |
| | 13.3, 5.1 Hz, 2H), 4.45 (s, 2H), 4.31 (q, J = | | | |
| | 16.8 Hz, 2H), 4.08 (d, J = 5.0 Hz, 1H), 4.02- | | | |
| | 3.92 (m, 1H), 3.52-3.44 (m, 1H), 3.00 (dd, J = | | | |
| | 12.0, 6.4 Hz, 4H), 2.96-2.78 (m, 3H), 2.77- | | | |
| | 2.59 (m, 4H), 2.56-2.31 (m, 3H), 2.07 | | | |
| | (ddd, J = 23.2, 14.8, 7.1 Hz, 5H), 1.82 (d, J = | | | |
| | 13.5 Hz, 2H), 1.64 (ddd, J = 24.5, 12.9, 7.0 | | | |
| | Hz, 5H), 1.40 (dd, J = 12.4, 8.3 Hz, 1H), 1.19 | | | |
| | (s, 2H). | | | |
| A23 | ¹H NMR (400 MHz, DMSO) δ 10.93 (s, 1H), | 782.9 | 782.8 | C |
| | 7.18 (d, J = 8.3 Hz, 1H), 7.06 (t, J = 5.9 Hz, | | | |
| | 3H), 7.01 (d, J = 8.5 Hz, 1H), 6.76-6.70 (m, | | | |
| | 2H), 6.55 (d, J = 8.8 Hz, 2H), 6.45 (dd, J = | | | |
| | 8.3, 2.3 Hz, 1H), 6.37 (d, J = 8.4 Hz, 1H), | | | |
| | 6.30 (d, J = 8.7 Hz, 1H), 6.11 (s, 1H), 5.03 | | | |
| | (dd, J = 13.3, 5.0 Hz, 1H), 4.64 (d, J = 5.1 Hz, | | | |
| | 1H), 4.40-4.33 (m, 1H), 4.26 (d, J = 16.9 | | | |
| | Hz, 1H), 4.10 (d, J = 16.9 Hz, 1H), 4.00- | | | |
| | 3.92 (m, 1H), 3.83 (d, J = 11.4 Hz, 1H), 3.56 | | | |
| | (s, 2H), 3.19 (t, J = 10.8 Hz, 2H), 2.99-2.87 | | | |
| | (m, 8H), 2.75 (t, J = 10.4 Hz, 1H), 2.57 (d, J = | | | |
| | 14.0 Hz, 3H), 2.43-2.32 (m, 1H), 2.15 (dt, J = | | | |
| | 19.4, 8.4 Hz, 4H), 1.99-1.91 (m, 1H), 1.75 | | | |
| | (t, J = 11.4 Hz, 3H), 1.68-1.54 (m, 2H), 1.25- | | | |
| | 1.12 (m, 2H). | | | |
| A24 | ¹H NMR (400 MHz, DMSO) δ 10.93 (s, 1H), | 782.9 | 782.8 | C |
| | 7.18 (d, J = 8.2 Hz, 1H), 7.07 (d, J = 6.8 Hz, | | | |
| | 3H), 7.01 (d, J = 8.5 Hz, 1H), 6.76-6.70 (m, | | | |
| | 2H), 6.55 (d, J = 8.7 Hz, 2H), 6.45 (dd, J = | | | |
| | 8.3, 2.2 Hz, 1H), 6.37 (d, J = 8.4 Hz, 1H), | | | |
| | 6.30 (d, J = 7.0 Hz, 1H), 6.11 (s, 1H), 5.03 | | | |
| | (dd, J = 13.2, 5.0 Hz, 1H), 4.64 (d, J = 5.1 Hz, | | | |
| | 1H), 4.36 (d, J = 8.3 Hz, 1H), 4.26 (d, J = | | | |
| | 16.9 Hz, 1H), 4.10 (d, J = 16.9 Hz, 1H), 4.01- | | | |
| | 3.91 (m, 1H), 3.82 (d, J = 11.5 Hz, 1H), | | | |
| | 3.56 (t, J = 10.1 Hz, 2H), 3.33 (s, 4H), 3.26- | | | |
| | 3.13 (m, 3H), 2.92-2.82 (m, 3H), 2.75 (t, J = | | | |
| | 10.4 Hz, 1H), 2.57 (d, J = 14.7 Hz, 3H), 2.45- | | | |
| | 2.31 (m, 1H), 2.26-2.04 (m, 4H), 2.01- | | | |
| | 1.89 (m, 1H), 1.69 (ddd, J = 46.1, 28.1, 9.8 | | | |
| | Hz, 5H), 1.27-1.11 (m, 2H). | | | |
| A25 | ¹H NMR (400 MHz, DMSO) δ 10.96 (s, 1H), | 808.0 | 808.0 | C |
| | 9.13 (s, 1H), 8.19 (s, 1H), 7.46 (s, 1H), 7.13 | | | |
| | (t, J = 7.6 Hz, 3H), 7.00 (s, 1H), 6.82 (d, J = | | | |
| | 6.7 Hz, 2H), 6.65 (d, J = 8.3 Hz, 1H), 6.59 (s, | | | |
| | 1H), 6.53 (d, J = 8.5 Hz, 2H), 6.48 (d, J = 8.2 | | | |
| | Hz, 1H), 6.20 (d, J = 8.5 Hz, 2H), 5.07 (dd, J = | | | |
| | 13.0, 5.2 Hz, 1H), 4.45 (s, 2H), 4.33 (d, J = | | | |
| | 16.9 Hz, 1H), 4.20 (d, J = 17.0 Hz, 1H), 4.12 | | | |
| | (d, J = 4.7 Hz, 1H), 3.86 (t, J = 7.6 Hz, 1H), | | | |
| | 3.46-3.41 (m, 3H), 3.02 (s, 4H), 2.98-2.78 | | | |
| | (m, 6H), 2.64 (d, J = 26.2 Hz, 1H), 2.34 (t, J = | | | |
| | 16.7 Hz, 3H), 2.17-2.05 (m, 1H), 1.93 (ddd, | | | |
| | J = 22.8, 14.6, 8.6 Hz, 6H), 1.71-1.57 (m, | | | |
| | 6H), 1.33 (dd, J = 12.6, 7.2 Hz, 1H). | | | |
| A26 | ¹H NMR (400 MHz, DMSO) δ 10.96 (s, 1H), | 808.0 | 808.0 | C |
| | 9.11 (s, 1H), 7.46 (s, 1H), 7.19-7.07 (m, | | | |
| | 3H), 7.00 (s, 1H), 6.82 (d, J = 6.4 Hz, 2H), | | | |
| | 6.65 (d, J = 8.4 Hz, 1H), 6.59 (s, 1H), 6.53 (d, | | | |
| | J = 8.8 Hz, 2H), 6.48 (dd, J = 8.4, 2.4 Hz, | | | |
| | 1H), 6.20 (d, J = 8.4 Hz, 2H), 5.07 (dd, J = | | | |
| | 13.2, 4.8 Hz, 1H), 4.49-4.42 (m, 2H), 4.33 | | | |
| | (d, J = 16.8 Hz, 1H), 4.20 (d, J = 17.2 Hz, | | | |
| | 1H), 4.12 (d, J = 4.8 Hz, 1H), 3.86 (d, J = 7.2 | | | |
| | Hz, 1H), 3.45 (d, J = 8.0 Hz, 2H), 3.03 (s, | | | |
| | 4H), 2.96-2.78 (m, 5H), 2.58 (d, J = 19.2 | | | |
| | Hz, 2H), 2.34 (dd, J = 21.6, 8.0 Hz, 3H), 2.10 | | | |
| | (dd, J = 12.0, 6.4 Hz, 1H), 2.01-1.84 (m, | | | |
| | 6H), 1.73-1.54 (m, 7H), 1.33 (dd, J = 12.4, | | | |
| | 8.4 Hz, 1H). | | | |
| A27 | ¹H NMR (400 MHz, DMSO) δ 10.76 (s, 1H), | 739.9 | 739.8 | C |
| | 7.17-7.09 (m, 3H), 6.94 (d, J = 8.4 Hz, 2H), | | | |
| | 6.82 (d, J = 6.4 Hz, 2H), 6.66-6.56 (m, 4H), | | | |
| | 6.55-6.45 (m, 3H), 6.19 (d, J = 8.8 Hz, 2H), | | | |
| | 5.64 (d, J = 7.6 Hz, 1H), 4.29-4.20 (m, 1H), | | | |
| | 4.12 (d, J = 4.8 Hz, 1H), 3.89-3.82 (m, 1H), | | | |
| | 3.45-3.38 (m, 1H), 3.31-3.23 (m, 2H), | | | |
| | 3.05-2.88 (m, 8H), 2.78-2.68 (m, 1H), | | | |
| | 2.61-2.53 (m, 2H), 2.37-2.29 (m, 3H), | | | |
| | 2.13-1.83 (m, 6H), 1.72-1.51 (m, 9H), | | | |
| | 1.32 (dd, J = 12.4, 8.4 Hz, 1H). | | | |
| A28 | ¹H NMR (400 MHz, DMSO) δ 10.76 (s, 1H), | 739.9 | 739.8 | C |
| | 7.17-7.08 (m, 3H), 6.94 (d, J = 8.4 Hz, 2H), | | | |
| | 6.82 (d, J = 6.4 Hz, 2H), 6.66-6.45 (m, 7H), | | | |
| | 6.19 (d, J = 8.8 Hz, 2H), 5.63 (d, J = 7.6 Hz, | | | |
| | 1H), 4.29-4.19 (m, 1H), 4.12 (d, J = 4.8 Hz, | | | |
| | 1H), 3.85 (t, J = 7.6 Hz, 1H), 3.45-3.39 (m, | | | |
| | 2H), 3.31-3.25 (m, 2H), 3.06-2.88 (m, | | | |
| | 8H), 2.78-2.66 (m, 1H), 2.63-2.52 (m, | | | |
| | 2H), 2.34-2.24 (m, 3H), 2.13-2.05 (m, | | | |
| | 2H), 2.01-1.79 (m, 4H), 1.69-1.52 (m, | | | |
| | 8H), 1.31 (dd, J = 12.4, 8.4 Hz, 1H). | | | |
| A29 | ¹H NMR (400 MHz, DMSO) δ 10.75 (s, 1H), | 739.9 | 739.8 | C |
| | 7.19-7.06 (m, 3H), 6.94 (d, J = 8.4 Hz, 2H), | | | |
| | 6.82 (d, J = 7.2 Hz, 2H), 6.66-6.56 (m, 4H), | | | |
| | 6.54-6.45 (m, 3H), 6.20 (d, J = 8.4 Hz, 2H), | | | |
| | 5.62 (d, J = 7.2 Hz, 1H), 4.28-4.22 (m, 1H), | | | |
| | 4.12 (d, J = 4.8 Hz, 1H), 3.84 (t, J = 7.6 Hz, | | | |
| | 1H), 3.44-3.40 (m, 2H), 3.30-3.25 (m, | | | |
| | 2H), 3.05-2.99 (m, 4H), 2.97-2.87 (m, | | | |
| | 4H), 2.79-2.66 (m, 2H), 2.62-2.58 (m, | | | |
| | 1H), 2.32-2.25 (m, 3H), 2.13-2.04 (m, | | | |
| | 2H), 1.98-1.81 (m, 4H), 1.71-1.68 (m, | | | |
| | 1H), 1.64-1.51 (m, 7H), 1.34-1.26 (m, 1H). | | | |
| A30 | ¹H NMR (400 MHz, DMSO) δ 10.76 (s, 1H), | 771.9 | 771.8 | D |
| | 8.20 (s, 1H), 7.08 (d, J = 6.8 Hz, 3H), 6.94 (d, | | | |
| | J = 8.4 Hz, 2H), 6.76 (d, J = 7.4 Hz, 2H), 6.62- | | | |
| | 6.54 (m, 4H), 6.47 (d, J = 8.2 Hz, 1H), 6.22- | | | |
| | 6.13 (m, 2H), 5.64 (d, J = 7.6 Hz, 1H), 4.66 | | | |
| | (d, J = 5.6 Hz, 1H), 4.25 (s, 1H), 3.27-3.23 | | | |
| | (m, 2H), 2.95 (s, 4H), 2.88 (s, 3H), 2.73 (s, | | | |
| | 3H), 2.59 (s, 1H), 2.41-2.39 (m, 1H), 2.31 | | | |
| | (d, J = 12.2 Hz, 1H), 2.11 (s, 1H), 1.98 (d, J = | | | |
| | 7.8 Hz, 4H), 1.85 (d, J = 8.0 Hz, 1H), 1.65 (d, | | | |
| | J = 11.0 Hz, 4H), 1.55 (s, 3H), 1.43 (d, J = 9.8 | | | |
| | Hz, 2H), 1.24 (s, 4H). | | | |
| A31 | ¹H NMR (400 MHz, DMSO) δ 10.77 (s, 1H), | 771.9 | 771.8 | D |
| | 8.21 (s, 1H), 7.08 (d, J = 7.2 Hz, 3H), 6.94 (d, | | | |
| | J = 8.2 Hz, 2H), 6.75 (d, J = 6.0 Hz, 2H), 6.60 | | | |
| | (d, J = 8.6 Hz, 3H), 6.54 (s, 1H), 6.47 (d, J = | | | |
| | 8.6 Hz, 1H), 6.25-6.10 (m, 2H), 5.64 (d, J = | | | |
| | 7.2 Hz, 1H), 4.66 (d, J = 5.4 Hz, 1H), 4.25 (s, | | | |
| | 1H), 3.26-3.22 (m, 3H), 2.95 (s, 2H), 2.88 | | | |
| | (s, 3H), 2.81-2.69 (m, 4H), 2.58 (d, J = 12.2 | | | |
| | Hz, 1H), 2.41 (s, 2H), 2.30 (s, 1H), 2.10 (s, | | | |
| | 2H), 2.02-1.96 (m, 3H), 1.85 (d, J = 7.8 Hz, | | | |
| | 1H), 1.64 (s, 4H), 1.54 (d, J = 6.2 Hz, 3H), | | | |
| | 1.41 (d, J = 10.8 Hz, 2H), 1.24 (s, 3H), 1.06- | | | |
| | 0.77 (m, 1H). | | | |
| A32 | ¹H NMR (400 MHz, DMSO) δ 10.76 (s, 1H), | 771.9 | 771.8 | D |
| | 9.13 (s, 1H), 8.16 (s, 1H), 7.08 (d, J = 6.6 Hz, | | | |
| | 3H), 6.94 (d, J = 10.0 Hz, 2H), 6.76 (d, J = | | | |
| | 7.6 Hz, 2H), 6.60 (d, J = 8.4 Hz, 3H), 6.54 (s, | | | |
| | 1H), 6.48 (s, 1H), 6.23-6.15 (m, 2H), 5.65 | | | |
| | (d, J = 7.6 Hz, 1H), 5.32 (s, 2H), 4.67 (s, 1H), | | | |
| | 4.26 (s, 1H), 2.95 (s, 3H), 2.89-2.84 (m, | | | |
| | 2H), 2.70 (d, J = 10.2 Hz, 6H), 2.33 (s, 2H), | | | |
| | 2.02 (d, J = 7.4 Hz, 4H), 1.87-1.80 (m, 1H), | | | |
| | 1.67 (s, 3H), 1.57 (s, 3H), 1.46-1.41 (m, | | | |
| | 3H), 1.26 (s, 4H), 0.85 (s, 2H). | | | |
| A33 | ¹H NMR (400 MHz, DMSO) δ 10.98 (s, 1H), | 781.9 | 781.8 | C |
| | 9.06 (s, 1H), 7.35 (s, 1H), 7.07 (d, J = 6.8 Hz, | | | |
| | 4H), 6.76-6.71 (m, 2H), 6.59-6.53 (m, | | | |
| | 2H), 6.45 (dd, J = 8.3, 2.3 Hz, 1H), 6.38 (d, J = | | | |
| | 8.5 Hz, 1H), 6.32 (d, J = 8.6 Hz, 1H), 6.13 | | | |
| | (s, 1H), 5.09 (dd, J = 13.2, 5.2 Hz, 1H), 4.65 | | | |
| | (d, J = 4.9 Hz, 1H), 4.55 (s, 2H), 4.37 (d, J = | | | |
| | 17.1 Hz, 1H), 4.25 (d, J = 17.0 Hz, 1H), 3.60 | | | |
| | (s, 2H), 3.26-3.18 (m, 2H), 3.00-2.85 (m, | | | |
| | 8H), 2.61 (dd, J = 21.4, 17.9 Hz, 4H), 2.37 | | | |
| | (dd, J = 19.3, 7.2 Hz, 2H), 2.24-2.09 (m, | | | |
| | 3H), 1.98 (dd, J = 6.8, 3.7 Hz, 2H), 1.89 (dd, J = | | | |
| | 13.0, 6.8 Hz, 1H), 1.79 (d, J = 9.7 Hz, 3H), | | | |
| | 1.63-1.56 (m, 1H), 1.33-1.20 (m, 3H). | | | |
| A34 | ¹H NMR (400 MHz, DMSO) δ 10.94 (s, 1H), | 782.9 | 782.2 | C |
| | 9.06 (s, 1H), 7.22-6.92 (m, 5H), 6.73 (d, J = | | | |
| | 5.8 Hz, 2H), 6.60-6.51 (m, 2H), 6.49-6.42 | | | |
| | (m, 1H), 6.40-6.28 (m, 2H), 6.13 (t, J = 6.0 | | | |
| | Hz, 1H), 5.03 (d, J = 13.0 Hz, 1H), 4.65 (d, J = | | | |
| | 5.2 Hz, 1H), 4.28-4.16 (m, 2H), 4.03- | | | |
| | 3.85 (m, 1H), 3.61 (dd, J = 21.7, 12.2 Hz, | | | |
| | 3H), 3.15 (d, J = 35.1 Hz, 4H), 3.00-2.85 | | | |
| | (m, 8H), 2.63 (d, J = 29.1 Hz, 2H), 2.33 (s, | | | |
| | 1H), 2.23-2.10 (m, 3H), 1.96 (dd, J = 10.7, | | | |
| | 5.9 Hz, 2H), 1.85-1.73 (m, 3H), 1.58 (dd, J = | | | |
| | 11.1, 8.2 Hz, 3H), 1.24 (s, 2H). | | | |
| A35 | ¹H NMR (400 MHz, MeOD) δ 7.32 (d, J = | 800.9 | 800.9 | D |
| | 8.3 Hz, 1H), 7.09 (d, J = 7.1 Hz, 3H), 7.03 (d, | | | |
| | J = 8.5 Hz, 1H), 6.81-6.76 (m, 2H), 6.65 | | | |
| | (dd, J = 8.2, 5.4 Hz, 2H), 6.54 (dd, J = 8.4, 2.4 | | | |
| | Hz, 1H), 6.29 (d, J = 13.6 Hz, 1H), 6.24 (d, J = | | | |
| | 7.5 Hz, 1H), 5.10 (dd, J = 13.3, 5.0 Hz, 1H), | | | |
| | 4.80 (d, J = 5.0 Hz, 1H), 4.41-4.27 (m, 3H), | | | |
| | 4.10-4.01 (m, 1H), 3.86 (d, J = 12.1 Hz, | | | |
| | 1H), 3.19-3.05 (m, 2H), 3.01 (d, J = 22.5 | | | |
| | Hz, 6H), 2.89 (dd, J = 13.1, 5.4 Hz, 2H), 2.82- | | | |
| | 2.75 (m, 1H), 2.72-2.58 (m, 2H), 2.50 (dd, | | | |
| | J = 13.2, 4.7 Hz, 1H), 2.35 (d, J = 7.0 Hz, | | | |
| | 2H), 2.28 (dd, J = 22.3, 10.0 Hz, 2H), 2.20- | | | |
| | 2.11 (m, 1H), 1.88 (dd, J = 7.6, 3.8 Hz, 3H), | | | |
| | 1.73 (dt, J = 14.3, 7.0 Hz, 2H), 1.38 (ddd, J = | | | |
| | 25.5, 18.7, 13.9 Hz, 5H). | | | |
| A36 | ¹H NMR (400 MHz, DMSO) δ 10.97 (s, 1H), | 781.9 | 781.8 | C |
| | 9.05 (s, 1H), 7.30 (t, J = 7.5 Hz, 2H), 7.07 (d, | | | |
| | J = 11.5 Hz, 3H), 6.74 (d, J = 7.6 Hz, 2H), | | | |
| | 6.55 (d, J = 11.7 Hz, 2H), 6.45 (d, J = 10.5 | | | |
| | Hz, 1H), 6.34 (dd, J = 25.3, 8.3 Hz, 2H), 6.12 | | | |
| | (d, J = 1.1 Hz, 1H), 5.09 (dd, J = 13.2, 4.8 Hz, | | | |
| | 1H), 4.65 (d, J = 5.0 Hz, 1H), 4.56 (s, 2H), | | | |
| | 4.38 (d, J = 17.1 Hz, 1H), 4.22 (d, J = 17.1 | | | |
| | Hz, 1H), 3.57 (d, J = 8.0 Hz, 2H), 3.25-3.19 | | | |
| | (m, 1H), 2.98-2.81 (m, 7H), 2.57 (t, J = 11.5 | | | |
| | Hz, 4H), 2.45-2.35 (m, 2H), 2.18 (dd, J = | | | |
| | 24.8, 14.4 Hz, 2H), 1.99 (dd, J = 18.0, 12.5 | | | |
| | Hz, 3H), 1.84-1.68 (m, 5H), 1.66-1.49 (m, | | | |
| | 2H), 1.33-1.14 (m, 3H). | | | |
| A37 | ¹H NMR (400 MHz, DMSO) δ 10.98 (s, 1H), | 781.9 | 781.8 | C |
| | 9.06 (s, 1H), 7.35-7.27 (m, 2H), 7.08 (d, J = | | | |
| | 6.7 Hz, 3H), 6.74 (d, J = 7.7 Hz, 2H), 6.56 | | | |
| | (dd, J = 9.3, 5.3 Hz, 2H), 6.49-6.43 (m, 1H), | | | |
| | 6.39 (d, J = 8.4 Hz, 1H), 6.32 (d, J = 9.2 Hz, | | | |
| | 1H), 6.14 (d, J = 1.4 Hz, 1H), 5.09 (dd, J = | | | |
| | 13.3, 5.1 Hz, 1H), 4.65 (d, J = 4.3 Hz, 2H), | | | |
| | 4.39 (d, J = 17.1 Hz, 1H), 4.23 (d, J = 17.1 | | | |
| | Hz, 1H), 3.64-3.54 (m, 3H), 3.22 (dd, J = | | | |
| | 10.9, 5.9 Hz, 2H), 3.08 (dd, J = 23.1, 8.9 Hz, | | | |
| | 3H), 2.96 (s, 3H), 2.88 (dd, J = 17.9, 11.8 Hz, | | | |
| | 3H), 2.63 (dd, J = 21.2, 11.2 Hz, 3H), 2.45- | | | |
| | 2.29 (m, 2H), 2.18 (ddd, J = 19.6, 15.1, 6.9 | | | |
| | Hz, 3H), 1.97 (dd, J = 18.0, 10.9 Hz, 4H), | | | |
| | 1.80 (dd, J = 17.0, 4.3 Hz, 2H), 1.64-1.49 | | | |
| | (m, 2H), 1.24 (s, 2H). | | | |
| A38 | ¹H NMR (400 MHz, DMSO) δ 10.97 (s, 1H), | 781.9 | 781.8 | C |
| | 9.05 (s, 1H), 7.43 (s, 1H), 7.06 (dd, J = 12.8, | | | |
| | 7.9 Hz, 4H), 6.77-6.69 (m, 2H), 6.56 (dd, J = | | | |
| | 7.2, 5.5 Hz, 2H), 6.45 (dd, J = 8.2, 2.4 Hz, | | | |
| | 1H), 6.33 (dt, J = 8.4, 5.0 Hz, 2H), 6.12 (d, J = | | | |
| | 1.7 Hz, 1H), 5.08 (dd, J = 13.3, 5.1 Hz, 1H), | | | |
| | 4.65 (d, J = 5.0 Hz, 1H), 4.50 (s, 2H), 4.36 (d, | | | |
| | J = 17.1 Hz, 1H), 4.23 (d, J = 17.0 Hz, 1H), | | | |
| | 3.57 (dd, J = 13.3, 5.1 Hz, 2H), 3.21 (dd, J = | | | |
| | 12.5, 6.8 Hz, 2H), 2.95 (s, 3H), 2.93-2.79 | | | |
| | (m, 3H), 2.60 (dd, J = 30.8, 17.5 Hz, 4H), | | | |
| | 2.43-2.32 (m, 2H), 2.16 (tdd, J = 23.5, 12.7, | | | |
| | 7.4 Hz, 2H), 2.05-1.94 (m, 3H), 1.91-1.63 | | | |
| | (m, 6H), 1.59 (dd, J = 15.2, 6.2 Hz, 1H), 1.27- | | | |
| | 1.17 (m, 3H). | | | |
| A39 | ¹H NMR (400 MHz, MeOD) δ 7.31 (d, J = | 800.9 | 800.9 | D |
| | 8.4 Hz, 1H), 7.08 (d, J = 6.8 Hz, 3H), 7.03 (d, | | | |
| | J = 8.5 Hz, 1H), 6.81-6.76 (m, 2H), 6.65 | | | |
| | (dd, J = 8.3, 5.3 Hz, 2H), 6.54 (dd, J = 8.3, 2.4 | | | |
| | Hz, 1H), 6.29 (d, J = 13.6 Hz, 1H), 6.23 (d, J = | | | |
| | 7.4 Hz, 1H), 5.10 (dd, J = 13.3, 5.0 Hz, 1H), | | | |
| | 4.80 (d, J = 5.6 Hz, 1H), 4.42-4.25 (m, 3H), | | | |
| | 4.05 (dd, J = 10.4, 8.9 Hz, 1H), 3.91-3.75 | | | |
| | (m, 1H), 3.28 (s, 1H), 3.15-2.94 (m, 8H), | | | |
| | 2.93-2.83 (m, 2H), 2.83-2.75 (m, 1H), | | | |
| | 2.71-2.58 (m, 2H), 2.49 (dd, J = 13.1, 4.6 | | | |
| | Hz, 1H), 2.35 (d, J = 6.9 Hz, 2H), 2.31-2.22 | | | |
| | (m, 2H), 2.18-2.11 (m, 1H), 1.88 (dd, J = | | | |
| | 16.5, 5.6 Hz, 3H), 1.72 (dd, J = 16.4, 7.1 Hz, | | | |
| | 2H), 1.37 (ddd, J = 24.1, 18.3, 6.1 Hz, 4H). | | | |
| A40 | ¹H NMR (400 MHz, DMSO) δ 10.93 (s, 1H), | 780.9 | 780.9 | A |
| | 8.24 (s, 1H), 7.20-7.08 (m, 4H), 7.01 (d, J = | | | |
| | 8.2 Hz, 1H), 6.82 (d, J = 6.6 Hz, 2H), 6.65- | | | |
| | 6.57 (m, 2H), 6.49-6.44 (m, 1H), 6.17 (d, J = | | | |
| | 8.4 Hz, 2H), 6.05 (d, J = 8.4 Hz, 2H), 5.08- | | | |
| | 4.97 (m, 1H), 4.35 (d, J = 10.2 Hz, 1H), 4.28 | | | |
| | (s, 1H), 4.24 (s, 1H), 4.14-4.06 (m, 2H), | | | |
| | 3.99-3.92 (m, 1H), 3.90-3.74 (m, 3H), | | | |
| | 3.74-3.70 (m, 1H), 3.65-3.59 (m, 1H), | | | |
| | 3.58-3.53 (m, 1H), 3.53-3.46 (m, 1H), | | | |
| | 3.03-2.83 (m, 5H), 2.81-2.63 (m, 2H), | | | |
| | 2.42-2.22 (m, 5H), 2.16-2.02 (m, 2H), | | | |
| | 2.00-1.61 (m, 5H). | | | |
| A41 | ¹H NMR (400 MHz, DMSO) δ 10.93 (s, 1H), | 780.9 | 780.9 | A |
| | 8.24 (s, 1H), 7.20-7.08 (m, 4H), 7.01 (d, J = | | | |
| | 8.2 Hz, 1H), 6.82 (d, J = 6.6 Hz, 2H), 6.65- | | | |
| | 6.57 (m, 2H), 6.49-6.44 (m, 1H), 6.17 (d, J = | | | |
| | 8.4 Hz, 2H), 6.05 (d, J = 8.4 Hz, 2H), 5.08- | | | |
| | 4.97 (m, 1H), 4.35 (d, J = 10.2 Hz, 1H), 4.28 | | | |
| | (s, 1H), 4.24 (s, 1H), 4.14-4.06 (m, 2H), | | | |
| | 3.99-3.92 (m, 1H), 3.90-3.74 (m, 3H), | | | |
| | 3.74-3.70 (m, 1H), 3.65-3.59 (m, 1H), | | | |
| | 3.58-3.53 (m, 1H), 3.53-3.46 (m, 1H), | | | |
| | 3.03-2.83 (m, 5H), 2.81-2.63 (m, 2H), | | | |
| | 2.42-2.22 (m, 5H), 2.16-2.02 (m, 2H), | | | |
| | 2.00-1.61 (m, 5H). | | | |
| A42 | ¹H NMR (400 MHz, DMSO) δ 10.93 (s, 1H), | 780.9 | 780.9 | D |
| | 8.24 (s, 1H), 7.20-7.08 (m, 4H), 7.01 (d, J = | | | |
| | 8.2 Hz, 1H), 6.82 (d, J = 6.6 Hz, 2H), 6.65- | | | |
| | 6.57 (m, 2H), 6.49-6.44 (m, 1H), 6.17 (d, J = | | | |
| | 8.4 Hz, 2H), 6.05 (d, J = 8.4 Hz, 2H), 5.08- | | | |
| | 4.97 (m, 1H), 4.35 (d, J = 10.2 Hz, 1H), 4.28 | | | |
| | (s, 1H), 4.24 (s, 1H), 4+J6.14-4.06 (m, 2H), | | | |
| | 3.99-3.92 (m, 1H), 3.90-3.74 (m, 3H), | | | |
| | 3.74-3.70 (m, 1H), 3.65-3.59 (m, 1H), | | | |
| | 3.58-+K:X 3.53 (m, 1H), 3.53-3.46 (m, | | | |
| | 1H), 3.03-2.83 (m, 5H), 2.81-2.63 (m, | | | |
| | 2H), 2.42-2.22 (m, 5H), 2.16-2.02 (m, | | | |
| | 2H), 2.00-1.61 (m, 5H). | | | |
| A43 | ¹H NMR (400 MHz, DMSO) δ 10.93 (s, 1H), | 780.9 | 780.9 | A |
| | 8.25 (s, 1H), 7.17-7.07 (m, 3H), 7.04 (s, | | | |
| | 1H), 6.93 (s, 1H), 6.82 (d, J = 6.6 Hz, 2H), | | | |
| | 6.65-6.57 (m, 2H), 6.50-6.45 (m, 1H), | | | |
| | 6.17 (d, J = 8.4 Hz, 2H), 6.05 (d, J = 8.4 Hz, | | | |
| | 2H), 5.06-4.98 (m, 1H), 4.32-4.19 (m, | | | |
| | 2H), 4.19-4.07 (m, 2H), 3.94-3.68 (m, | | | |
| | 5H), 3.62 (t, J = 7.4 Hz, 2H), 3.58-3.45 (m, | | | |
| | 3H), 3.04-2.82 (m, 6H), 2.82-2.71 (m, | | | |
| | 1H), 2.69-2.65 (m, 1H), 2.42-2.21 (m, | | | |
| | 4H), 2.16-2.01 (m, 2H), 2.01-1.91 (m, | | | |
| | 1H), 1.90-1.80 (m, 1H), 1.79-1.64 (m, 2H). | | | |
| A44 | ¹H NMR (400 MHz, DMSO) δ 10.78 (s, 1H), | 787.9 | 787.8 | E |
| | 8.17 (s, 1H), 7.20-7.06 (m, 3H), 7.01 (d, J = | | | |
| | 8.2 Hz, 2H), 6.82 (d, J = 6.8 Hz, 2H), 6.70- | | | |
| | 6.57 (m, 4H), 6.57-6.44 (m, 3H), 6.19 (d, J = | | | |
| | 8.4 Hz, 2H), 5.85 (d, J = 7.6 Hz, 1H), 4.35- | | | |
| | 4.24 (m, 1H), 4.12 (d, J = 4.8 Hz, 1H), 3.86- | | | |
| | 3.78 (m, 1H), 3.42 (t, J = 7.8 Hz, 1H), 3.30- | | | |
| | 3.24 (m, 1H), 3.06-2.90 (m, 7H), 2.78- | | | |
| | 2.69 (m, 1H), 2.62-2.52 (m, 3H), 2.45- | | | |
| | 2.26 (m, 3H), 2.17-2.04 (m, 5H), 1.92- | | | |
| | 1.55 (m, 8H), 1.36-1.26 (m, 1H). | | | |
| A45 | ¹H NMR (400 MHz, DMSO) δ 10.78 (s, 1H), | 787.9 | 787.8 | E |
| | 8.21 (s, 1H), 7.20-7.05 (m, 3H), 7.01 (d, J = | | | |
| | 8.0 Hz, 2H), 6.82 (d, J = 6.8 Hz, 2H), 6.68- | | | |
| | 6.56 (m, 4H), 6.56-6.43 (m, 3H), 6.19 (d, J = | | | |
| | 8.2 Hz, 2H), 5.85 (d, J = 7.2 Hz, 1H), 4.33- | | | |
| | 4.25 (m, 1H), 4.12 (d, J = 4.2 Hz, 1H), 3.86- | | | |
| | 3.78 (m, 1H), 3.42 (t, J = 7.6 Hz, 1H), 3.27 (d, | | | |
| | J = 10.0 Hz, 1H), 3.07-2.88 (m, 7H), 2.78 | | | |
| | 2.68 (m, 1H), 2.63-2.52 (m, 3H), 2.44- | | | |
| | 2.26 (m, 3H), 2.19-2.03 (m, 5H), 1.92- | | | |
| | 1.54 (m, 8H), 1.35-1.26 (m, 1H). | | | |
| A46 | ¹H NMR (400 MHz, DMSO) δ 10.76 (s, 1H), | 771.4 | 771.4 | D |
| | 8.21 (s, 1H), 7.08 (t, J = 6.6 Hz, 3H), 6.94 (d, | | | |
| | J = 8.6 Hz, 2H), 6.76 (d, J = 7.6 Hz, 2H), 6.60 | | | |
| | (d, J = 8.6 Hz, 3H), 6.55 (d, J = 8.4 Hz, 1H), | | | |
| | 6.49-6.44 (m, 1H), 6.22-6.13 (m, 2H), | | | |
| | 5.64 (d, J = 7.4 Hz, 1H), 4.66 (d, J = 5.2 Hz, | | | |
| | 1H), 4.25 (s, 1H), 2.95 (s, 4H), 2.89 (d, J = | | | |
| | 11.8 Hz, 4H), 2.80 (s, 1H), 2.76-2.68 (m, | | | |
| | 2H), 2.62-2.54 (m, 1H), 2.40 (d, J = 6.4 Hz, | | | |
| | 2H), 2.29 (s, 1H), 2.20-2.06 (m, 2H), 2.02- | | | |
| | 1.91 (m, 5H), 1.87-1.77 (m, 1H), 1.65 (d, J = | | | |
| | 10.6 Hz, 5H), 1.55 (s, 2H), 1.43 (d, J = 9.4 | | | |
| | Hz, 2H), 1.24 (s, 4H). | | | |
| A47 | ¹H NMR (400 MHz, DMSO) δ 10.93 (s, 1H), | 810.4 | 810.3 | C |
| | 8.97 (s, 1H), 7.29 (t, J = 7.4 Hz, 2H), 7.25- | | | |
| | 7.07 (m, 5H), 7.01 (dd, J = 8.3, 3.8 Hz, 1H), | | | |
| | 6.55 (d, J = 8.0 Hz, 1H), 6.42 (d, J = 11.9 Hz, | | | |
| | 2H), 6.29 (s, 1H), 6.17 (s, 1H), 5.03 (dd, J = | | | |
| | 13.3, 5.1 Hz, 1H), 4.36-4.24 (m, 3H), 4.10 | | | |
| | (d, J = 17.0 Hz, 1H), 3.96 (dd, J = 17.1, 7.8 | | | |
| | Hz, 2H), 3.84 (s, 3H), 3.49 (dd, J = 13.1, 6.0 | | | |
| | Hz, 2H), 3.39 (d, J = 5.5 Hz, 3H), 3.13 (s, | | | |
| | 1H), 2.87 (d, J = 12.5 Hz, 3H), 2.75-2.60 | | | |
| | (m, 4H), 2.26 (dd, J = 15.2, 8.1 Hz, 3H), 1.93 | | | |
| | (s, 2H), 1.72 (d, J = 8.8 Hz, 4H), 1.29 (s, 2H), | | | |
| | 0.85 (t, J = 6.6 Hz, 2H). | | | |
| A48 | ¹H NMR (400 MHz, DMSO) δ 10.93 (s, 1H), | 810.4 | 810.3 | C |
| | 8.35 (s, 1H), 7.43-7.22 (m, 3H), 7.17 (td, J = | | | |
| | 7.3, 3.7 Hz, 4H), 7.01 (dd, J = 8.3, 4.3 Hz, | | | |
| | 1H), 6.55 (d, J = 8.2 Hz, 1H), 6.45-6.39 (m, | | | |
| | 2H), 6.29 (s, 1H), 6.17 (s, 1H), 5.03 (dd, J = | | | |
| | 13.2, 5.0 Hz, 1H), 4.30 (dd, J = 25.0, 8.7 Hz, | | | |
| | 3H), 4.10 (d, J = 16.9 Hz, 1H), 3.98-3.92 | | | |
| | (m, 1H), 3.78 (d, J = 6.9 Hz, 2H), 3.49-3.43 | | | |
| | (m, 4H), 3.14 (d, J = 8.5 Hz, 2H), 2.89 (dd, J = | | | |
| | 10.5, 7.0 Hz, 3H), 2.66 (ddd, J = 31.2, 17.1, | | | |
| | 10.5 Hz, 4H), 2.45-2.27 (m, 3H), 2.22 (d, J = | | | |
| | 7.3 Hz, 2H), 2.09 (dd, J = 15.9, 8.7 Hz, 2H), | | | |
| | 1.91 (dd, J = 27.4, 5.9 Hz, 3H), 1.76-1.59 | | | |
| | (m, 5H). | | | |
| A49 | ¹H NMR (400 MHz, MeOD) δ 7.14 (d, J = | 739.4 | 739.3 | C |
| | 7.0 Hz, 3H), 7.12-7.03 (m, 4H), 6.88-6.81 | | | |
| | (m, 2H), 6.75 (d, J = 8.5 Hz, 2H), 6.72-6.66 | | | |
| | (m, 2H), 6.62-6.51 (m, 3H), 4.35 (d, J = 5.3 | | | |
| | Hz, 1H), 4.29 (dd, J = 11.8, 4.9 Hz, 1H), 4.20- | | | |
| | 4.14 (m, 1H), 3.76-3.63 (m, 3H), 3.60- | | | |
| | 3.50 (m, 2H), 3.46-3.41 (m, 2H), 3.29 (d, J = | | | |
| | 6.8 Hz, 2H), 3.17-2.99 (m, 4H), 2.96- | | | |
| | 2.87 (m, 1H), 2.82-2.75 (m, 2H), 2.38- | | | |
| | 2.19 (m, 3H), 2.10 (d, J = 13.4 Hz, 3H), 2.02- | | | |
| | 1.92 (m, 5H), 1.84 (d, J = 8.4 Hz, 1H), 1.67 | | | |
| | (dd, J = 12.5, 9.1 Hz, 1H), 1.39-1.29 (m, 3H). | | | |
| A50 | ¹H NMR (400 MHz, MeOD) δ 7.16-7.04 | 739.4 | 739.3 | C |
| | (m, 5H), 6.82 (dd, J = 7.3, 2.0 Hz, 2H), 6.73 | | | |
| | (dd, J = 11.8, 8.5 Hz, 3H), 6.66 (dd, J = 9.1, | | | |
| | 5.6 Hz, 3H), 6.54 (dd, J = 8.3, 2.5 Hz, 1H), | | | |
| | 6.33 (d, J = 8.6 Hz, 2H), 4.29 (dd, J = 11.8, | | | |
| | 4.9 Hz, 1H), 4.20 (d, J = 5.0 Hz, 1H), 4.14- | | | |
| | 4.09 (m, 1H), 3.62 (dd, J = 19.5, 11.2 Hz, | | | |
| | 3H), 3.25 (d, J = 6.7 Hz, 2H), 3.12 (d, J = 4.7 | | | |
| | Hz, 5H), 3.04 (d, J = 5.7 Hz, 2H), 2.90-2.72 | | | |
| | (m, 4H), 2.38-2.30 (m, 1H), 2.27-2.17 (m, | | | |
| | 2H), 2.08 (d, J = 15.6 Hz, 2H), 1.96 (d, J = | | | |
| | 13.0 Hz, 3H), 1.78 (tt, J = 13.4, 6.7 Hz, 5H), | | | |
| | 1.55 (dd, J = 12.6, 8.8 Hz, 1H), 1.33 (d, J = | | | |
| | 16.2 Hz, 2H). | | | |
| A51 | ¹H NMR (400 MHz, DMSO) δ 10.75 (s, 1H), | 739.4 | 739.3 | C |
| | 7.16-7.10 (m, 3H), 6.95-6.89 (m, 2H), | | | |
| | 6.82 (d, J = 7.2 Hz, 2H), 6.67-6.58 (m, 4H), | | | |
| | 6.54-6.46 (m, 3H), 6.20 (d, J = 8.4 Hz, 2H), | | | |
| | 5.62 (d, J = 7.6 Hz, 1H), 4.29-4.20 (m, 1H), | | | |
| | 4.12 (d, J = 4.8 Hz, 1H), 3.84 (d, J = 7.6 Hz, | | | |
| | 1H), 3.44-3.40 (m, 2H), 3.30-3.26 (m, | | | |
| | 2H), 3.05-2.92 (m, 8H), 2.77-2.67 (m, | | | |
| | 1H), 2.62-2.56 (m, 1H), 2.33-2.26 (m, | | | |
| | 3H), 2.14-2.06 (m, 2H), 2.00-1.84 (m, | | | |
| | 4H), 1.68-1.52 (m, 9H), 1.31 (dd, J = 12.4, | | | |
| | 8.4 Hz, 1H). | | | |
| A52 | ¹H NMR (400 MHz, MeOD) δ 7.31 (d, J = | 838.4 | 838.4 | C |
| | 8.3 Hz, 1H), 7.05 (dt, J = 12.0, 6.0 Hz, 4H), | | | |
| | 6.78-6.72 (m, 2H), 6.64 (d, J = 8.3 Hz, 2H), | | | |
| | 6.54-6.48 (m, 2H), 6.43 (dd, J = 8.5, 2.1 Hz, | | | |
| | 1H), 6.21 (d, J = 2.1 Hz, 1H), 5.10 (dd, J = | | | |
| | 13.2, 5.1 Hz, 1H), 4.78 (d, J = 5.3 Hz, 1H), | | | |
| | 4.40-4.34 (m, 2H), 4.05 (dd, J = 12.5, 6.7 | | | |
| | Hz, 2H), 3.86 (d, J = 11.8 Hz, 1H), 3.61 (t, J = | | | |
| | 8.0 Hz, 1H), 3.23 (s, 1H), 3.15 (s, 3H), 3.03 | | | |
| | (s, 3H), 3.00 (d, J = 5.5 Hz, 2H), 2.89 (dd, J = | | | |
| | 12.8, 4.9 Hz, 2H), 2.82-2.76 (m, 1H), 2.74- | | | |
| | 2.66 (m, 1H), 2.48 (d, J = 7.2 Hz, 2H), 2.30 | | | |
| | (s, 1H), 2.20-2.05 (m, 3H), 1.89 (d, J = 18.7 | | | |
| | Hz, 1H), 1.80 (s, 4H), 1.65 (s, 2H), 1.49 (dd, J = | | | |
| | 12.6, 8.3 Hz, 1H), 1.33 (d, J = 18.0 Hz, 5H). | | | |
| A53 | ¹H NMR (400 MHz, MeOD) δ 7.35 (d, J = | 838.4 | 838.4 | C |
| | 8.3 Hz, 1H), 7.12-7.04 (m, 4H), 6.75 (dd, J = | | | |
| | 6.6, 2.8 Hz, 2H), 6.69-6.55 (m, 4H), 6.51 | | | |
| | (dd, J = 8.4, 2.6 Hz, 1H), 6.32 (s, 1H), 5.10 | | | |
| | (dd, J = 13.3, 5.0 Hz, 1H), 4.44-4.39 (m, | | | |
| | 1H), 4.36 (d, J = 9.1 Hz, 1H), 4.09 (d, J = 6.3 | | | |
| | Hz, 2H), 3.63 (t, J = 7.8 Hz, 1H), 3.48 (d, J = | | | |
| | 16.7 Hz, 1H), 3.26 (s, 4H), 3.06 (s, 3H), 3.03- | | | |
| | 2.86 (m, 5H), 2.85-2.72 (m, 3H), 2.56- | | | |
| | 2.36 (m, 2H), 2.30 (dd, J = 11.2, 6.6 Hz, 1H), | | | |
| | 2.24-2.15 (m, 2H), 2.05 (s, 1H), 1.87 (s, | | | |
| | 4H), 1.73-1.51 (m, 3H), 1.38-1.29 (m, 5H). | | | |
| A54 | ¹H NMR (400 MHz, DMSO) δ 10.97 (s, 1H), | 837.4 | 837.4 | D |
| | 9.05 (s, 1H), 7.40 (d, J = 7.6 Hz, 1H), 7.26 (d, | | | |
| | J = 7.6 Hz, 1H), 7.07 (d, J = 6.6 Hz, 3H), 6.72 | | | |
| | (d, J = 9.1 Hz, 2H), 6.56 (d, J = 8.5 Hz, 2H), | | | |
| | 6.45 (d, J = 10.7 Hz, 1H), 6.37 (d, J = 8.5 Hz, | | | |
| | 1H), 6.31 (d, J = 6.3 Hz, 1H), 6.11 (s, 1H), | | | |
| | 5.08 (dd, J = 12.0, 4.2 Hz, 1H), 4.64 (d, J = | | | |
| | 5.3 Hz, 1H), 4.52 (s, 2H), 4.37 (d, J = 17.0 | | | |
| | Hz, 1H), 4.21 (d, J = 16.9 Hz, 1H), 3.88 (t, J = | | | |
| | 7.8 Hz, 1H), 3.47-3.42 (m, 1H), 3.20 (dd, J = | | | |
| | 12.9, 6.6 Hz, 1H), 3.08 (s, 4H), 2.94 (s, | | | |
| | 3H), 2.88 (dd, J = 19.0, 11.1 Hz, 4H), 2.64 (d, | | | |
| | J = 25.1 Hz, 2H), 2.34 (d, J = 7.0 Hz, 2H), | | | |
| | 2.22-2.14 (m, 1H), 2.00 (d, J = 15.9 Hz, | | | |
| | 2H), 1.89 (dd, J = 27.6, 14.3 Hz, 4H), 1.71- | | | |
| | 1.64 (m, 4H), 1.60 (d, J = 9.6 Hz, 4H), 1.39- | | | |
| | 1.22 (m, 2H). | | | |
| A55 | ¹H NMR (400 MHz, DMSO) δ 10.97 (s, 1H), | 837.4 | 837.4 | D |
| | 8.41 (s, 1H), 7.40 (d, J = 7.6 Hz, 1H), 7.26 (d, | | | |
| | J = 7.5 Hz, 1H), 7.06 (s, 3H), 6.72 (d, J = 6.2 | | | |
| | Hz, 2H), 6.55 (d, J = 8.2 Hz, 2H), 6.45 (d, J = | | | |
| | 8.6 Hz, 1H), 6.36 (d, J = 8.7 Hz, 1H), 6.32 (s, | | | |
| | 1H), 6.11 (s, 1H), 5.06 (s, 1H), 4.64 (d, J = | | | |
| | 4.8 Hz, 1H), 4.52 (s, 2H), 4.37 (d, J = 17.6 | | | |
| | Hz, 1H), 4.21 (d, J = 16.8 Hz, 1H), 3.88 (s, | | | |
| | 1H), 3.08 (s, 4H), 2.94 (s, 3H), 2.88-2.76 | | | |
| | (m, 4H), 2.67 (s, 2H), 2.32 (s, 3H), 2.20 (s, | | | |
| | 2H), 1.99 (s, 2H), 1.97-1.90 (m, 4H), 1.65 | | | |
| | (s, 4H), 1.61 (s, 4H), 1.35 (s, 1H), 1.24 (s, 1H). | | | |
| A56 | ¹H NMR (400 MHz, DMSO) δ 10.93 (s, 1H), | 838.4 | 838.4 | C |
| | 9.05 (s, 1H), 7.06 (t, J = 5.8 Hz, 4H), 6.93 (s, | | | |
| | 1H), 6.74-6.70 (m, 2H), 6.55 (d, J = 8.2 Hz, | | | |
| | 2H), 6.45 (dd, J = 8.4, 2.5 Hz, 1H), 6.36 (d, J = | | | |
| | 8.4 Hz, 1H), 6.31 (d, J = 7.9 Hz, 1H), 6.11 | | | |
| | (s, 1H), 5.07-4.99 (m, 1H), 4.64 (d, J = 4.9 | | | |
| | Hz, 1H), 4.35-3.99 (m, 4H), 3.90 (s, 3H), | | | |
| | 3.45 (s, 1H), 3.20 (d, J = 14.6 Hz, 2H), 3.08 | | | |
| | (s, 4H), 2.94 (s, 3H), 2.67 (d, J = 1.8 Hz, 2H), | | | |
| | 2.33 (d, J = 1.8 Hz, 3H), 2.11 (d, J = 28.9 Hz, | | | |
| | 3H), 1.96 (d, J = 5.5 Hz, 3H), 1.65 (s, 4H), | | | |
| | 1.60 (s, 4H), 1.35 (s, 1H), 1.24 (s, 1H). | | | |
| A57 | ¹H NMR (400 MHz, DMSO) δ 10.94 (s, 1H), | 838.4 | 838.4 | C |
| | 9.06 (s, 1H), 7.19 (s, 1H), 7.07 (d, J = 6.6 Hz, | | | |
| | 4H), 6.75-6.69 (m, 2H), 6.58-6.53 (m, | | | |
| | 2H), 6.45 (dd, J = 8.3, 2.4 Hz, 1H), 6.36 (t, J = | | | |
| | 10.9 Hz, 2H), 6.13 (s, 1H), 5.04 (dd, J = | | | |
| | 13.3, 4.9 Hz, 1H), 4.65 (d, J = 5.1 Hz, 1H), | | | |
| | 4.31 (d, J = 14.0 Hz, 2H), 4.22 (d, J = 20.4 | | | |
| | Hz, 3H), 3.98 (s, 2H), 3.53 (d, J = 53.1 Hz, | | | |
| | 5H), 3.22 (d, J = 13.1 Hz, 3H), 3.10 (s, 5H), | | | |
| | 2.94 (s, 3H), 2.89 (d, J = 11.8 Hz, 3H), 2.64 | | | |
| | (d, J = 27.3 Hz, 2H), 2.34 (d, J = 12.2 Hz, | | | |
| | 1H), 2.22-2.06 (m, 2H), 1.96 (d, J = 5.3 Hz, | | | |
| | 1H), 1.65 (d, J = 13.9 Hz, 6H). | | | |
| A58 | ¹H NMR (400 M Hz, DMSO-d₆) δ 10.85 (s, | 713.4 | 713.4 | |
| | 1H), 8.72 (d, J = 8.4 Hz, 1H), 8.42-8.06 (m, | | | |
| | 2H), 7.86 (d, J = 8.8 Hz, 1H), 7.40 (dd, J = | | | |
| | 8.8, 2.8 Hz, 1H), 7.22-7.05 (m, 3H), 6.83 (d, | | | |
| | J = 6.4 Hz, 2H), 6.67-6.42 (m, 5H), 6.20 (d, | | | |
| | J = 8.8 Hz, 2H), 4.82-4.68 (m, 1H), 4.12 (d, | | | |
| | J = 4.8 Hz, 1H), 3.70-3.13 (m, 9H), 3.01- | | | |
| | 2.73 (m, 3H), 2.61-2.52 (m, 1H), 2.49- | | | |
| | 2.42 (m, 4H), 2.24-1.92 (m, 5H), 1.80- | | | |
| | 1.55 (m, 4H), 1.25-1.06 (m, 2H). | | | |
| A59 | ¹H NMR (400 M Hz, DMSO-d₆) δ 10.78 (s, | 757.4 | 757.4 | |
| | 1H), 8.35 (s, 1H), 7.18-7.06 (m, 3H), 6.98 | | | |
| | (t, J = 8.8 Hz, 1H), 6.82 (d, J = 6.6 Hz, 2H), | | | |
| | 6.67-6.58 (m, 2H), 6.55-6.37 (m, 5H), | | | |
| | 6.19 (d, J = 8.6 Hz, 2H), 5.99 (d, J = 7.8 Hz, | | | |
| | 1H), 4.36-4.25 (m, 1H), 4.12 (d, J = 4.6 Hz, | | | |
| | 1H), 3.87-3.80 (m, 1H), 3.43-3.40 (m, | | | |
| | 1H), 3.29-3.25 (m, 1H), 3.12-2.78 (m, | | | |
| | 9H), 2.77-2.65 (m, 1H), 2.62-2.53 (m, | | | |
| | 2H), 2.32-2.25 (m, 2H), 2.14-2.03 (m, | | | |
| | 2H), 2.01-1.79 (m, 4H), 1.73-1.65 (m, | | | |
| | 1H), 1.64-1.50 (m, 8H), 1.36-1.26 (m, 1H). | | | |
| A60 | ¹H NMR (400 M Hz, DMSO-d₆) δ 10.78 (s, | 757.4 | 757.4 | |
| | 1H), 8.35 (s, 1H), 7.18-7.06 (m, 3H), 6.98 | | | |
| | (t, J = 8.8 Hz, 1H), 6.82 (d, J = 6.6 Hz, 2H), | | | |
| | 6.67-6.58 (m, 2H), 6.55-6.37 (m, 5H), | | | |
| | 6.19 (d, J = 8.6 Hz, 2H), 5.99 (d, J = 7.8 Hz, | | | |
| | 1H), 4.36-4.25 (m, 1H), 4.12 (d, J = 4.6 Hz, | | | |
| | 1H), 3.87-3.80 (m, 1H), 3.43-3.40 (m, | | | |
| | 1H), 3.29-3.25 (m, 1H), 3.12-2.78 (m, | | | |
| | 9H), 2.77-2.65 (m, 1H), 2.62-2.53 (m, | | | |
| | 2H), 2.32-2.25 (m, 2H), 2.14-2.03 (m, | | | |
| | 2H), 2.01-1.79 (m, 4H), 1.73-1.65 (m, | | | |
| | 1H), 1.64-1.50 (m, 8H), 1.36-1.26 (m, 1H). | | | |
| A61 | ¹H NMR (400 M Hz, DMSO-d₆) δ 10.78 (s, | 757.4 | 757.4 | |
| | 1H), 8.35 (s, 1H), 7.18-7.06 (m, 3H), 6.98 | | | |
| | (t, J = 8.8 Hz, 1H), 6.82 (d, J = 6.6 Hz, 2H), | | | |
| | 6.67-6.58 (m, 2H), 6.55-6.37 (m, 5H), | | | |
| | 6.19 (d, J = 8.6 Hz, 2H), 5.99 (d, J = 7.8 Hz, | | | |
| | 1H), 4.36-4.25 (m, 1H), 4.12 (d, J = 4.6 Hz, | | | |
| | 1H), 3.87-3.80 (m, 1H), 3.43-3.40 (m, | | | |
| | 1H), 3.29-3.25 (m, 1H), 3.12-2.78 (m, | | | |
| | 9H), 2.77-2.65 (m, 1H), 2.62-2.53 (m, | | | |
| | 2H), 2.32-2.25 (m, 2H), 2.14-2.03 (m, | | | |
| | 2H), 2.01-1.79 (m, 4H), 1.73-1.65 (m, | | | |
| | 1H), 1.64-1.50 (m, 8H), 1.36-1.26 (m, 1H). | | | |
| A62 | ¹H NMR (400 M Hz, DMSO-d₆) δ 10.78 (s, | 757.4 | 757.4 | |
| | 1H), 8.35 (s, 1H), 7.18-7.06 (m, 3H), 6.98 | | | |
| | (t, J = 8.8 Hz, 1H), 6.82 (d, J = 6.6 Hz, 2H), | | | |
| | 6.67-6.58 (m, 2H), 6.55-6.37 (m, 5H), | | | |
| | 6.19 (d, J = 8.6 Hz, 2H), 5.99 (d, J = 7.8 Hz, | | | |
| | 1H), 4.36-4.25 (m, 1H), 4.12 (d, J = 4.6 Hz, | | | |
| | 1H), 3.87-3.80 (m, 1H), 3.43-3.40 (m, | | | |
| | 1H), 3.29-3.25 (m, 1H), 3.12-2.78 (m, | | | |
| | 9H), 2.77-2.65 (m, 1H), 2.62-2.53 (m, | | | |
| | 2H), 2.32-2.25 (m, 2H), 2.14-2.03 (m, | | | |
| | 2H), 2.01-1.79 (m, 4H), 1.73-1.65 (m, | | | |
| | 1H), 1.64-1.50 (m, 8H), 1.36-1.26 (m, 1H). | | | |
| A63 | ¹H NMR (400 M Hz, MeOD) δ 7.15-7.09 (m, | 792.5 | 792.5 | |
| | 4H), 7.02 (s, 1H), 6.82 (d, J = 7.9 Hz, 2H), | | | |
| | 6.71 (d, J = 8.3 Hz, 1H), 6.65 (s, 1H), 6.53 (d, | | | |
| | J = 10.9 Hz, 1H), 6.26 (d, J = 8.5 Hz, 2H), | | | |
| | 6.15 (d, J = 8.5 Hz, 2H), 5.09 (dd, J = 13.0, | | | |
| | 4.9 Hz, 1H), 4.35 (s, 1H), 4.29 (d, J = 13.6 | | | |
| | Hz, 1H), 4.17 (d, J = 4.5 Hz, 1H), 3.98 (d, J = | | | |
| | 9.2 Hz, 1H), 3.83 (s, 1H), 3.49 (s, 2H), 3.42 | | | |
| | (s, 2H), 3.09-2.89 (m, 6H), 2.82-2.76 (m, 1H), | | | |
| | 2.48 (dd, J = 17.3, 9.2 Hz, 1H), 2.24 (dd, J = | | | |
| | 14.4, 6.3 Hz, 4H), 1.94 (dd, J = 9.8, 6.8 Hz, | | | |
| | 2H), 1.87-1.76 (m, 4H), 1.57 (ddd, J = 29.1, | | | |
| | 16.6, 6.1 Hz, 4H), 1.38-1.30 (m, 3H), 1.10- | | | |
| | 0.99 (m, 2H). | | | |
| A64 | ¹H NMR (400 M Hz, MeOD) δ 7.14-7.07 (m, | 771.5 | 771.5 | |
| | 5H), 6.82-6.77 (m, 2H), 6.74-6.62 (m, 4H), | | | |
| | 6.50 (dd, J = 8.4, 2.6 Hz, 1H), 6.23 (d, J = 8.5 | | | |
| | Hz, 2H), 6.12 (d, J = 8.5 Hz, 2H), 4.28 (dd, J = | | | |
| | 11.8, 4.9 Hz, 1H), 4.14 (d, J = 4.8 Hz, 1H), | | | |
| | 3.49-3.43 (m, 2H), 3.39 (q, J = 7.0 Hz, 2H), | | | |
| | 3.27 (s, 1H), 3.04-2.93 (m, 2H), 2.78 (dd, J = | | | |
| | 12.3, 5.3 Hz, 1H), 2.75-2.64 (m, 3H), 2.40- | | | |
| | 2.31 (m, 3H), 2.27-2.16 (m, 2H), 2.10-1.99 | | | |
| | (m, 2H), 1.92 (dd, J = 12.6, 4.2 Hz, 3H), 1.81- | | | |
| | 1.70 (m, 3H), 1.63-1.47 (m, 3H), 1.31 (dd, J = | | | |
| | 17.4, 4.5 Hz, 2H), 1.09-0.97 (m, 2H). | | | |
| A65 | ¹H NMR (400 M Hz, MeOD) δ 8.55 (s, 1H), | 723.5 | 723.5 | |
| | 7.13 (d, J = 6.9 Hz, 3H), 7.06 (d, J = 8.5 Hz, | | | |
| | 2H), 6.83 (d, J = 7.4 Hz, 2H), 6.70 (dd, J = | | | |
| | 21.1, 12.3 Hz, 4H), 6.53 (d, J = 8.3 Hz, 1H), | | | |
| | 6.27 (d, J = 8.3 Hz, 2H), 6.15 (d, J = 8.5 Hz, | | | |
| | 2H), 5.37 (s, 1H), 4.28 (dd, J = 11.7, 5.0 Hz, | | | |
| | 1H), 4.17 (d, J = 4.7 Hz, 1H), 3.54-3.46 (m, | | | |
| | 2H), 3.44 (d, J = 8.9 Hz, 2H), 3.05-3.00 (m, | | | |
| | 1H), 2.85-2.74 (m, 2H), 2.72-2.63 (m, 3H), | | | |
| | 2.38-2.31 (m, 1H), 2.21 (t, J = 7.4 Hz, 1H), | | | |
| | 2.05 (d, J = 4.9 Hz, 1H), 1.98 (d, J = 14.5 Hz, | | | |
| | 2H), 1.95-1.90 (m, 3H), 1.78 (t, J = 9.6 Hz, | | | |
| | 3H), 1.61-1.53 (m, 2H), 1.33 (d, J = 17.9 Hz, | | | |
| | 7H), 1.12 (dd, J = 24.0, 9.7 Hz, 2H), 0.92 (t, J = | | | |
| | 6.1 Hz, 1H). | | | |
| A66 | ¹H NMR (400 M Hz, MeOD) δ 7.11 (t, J = 5.9 | 723.5 | 723.5 | |
| | Hz, 5H), 7.04 (d, J = 8.5 Hz, 2H), 6.83 (d, J = | | | |
| | 7.7 Hz, 2H), 6.72 (d, J = 8.5 Hz, 2H), 6.66 (d, | | | |
| | J = 8.6 Hz, 2H), 6.58 (d, J = 8.4 Hz, 2H), 6.52 | | | |
| | (dd, J = 8.3, 2.3 Hz, 1H), 4.34 (d, J = 5.0 Hz, | | | |
| | 1H), 4.27 (dd, J = 11.9, 4.9 Hz, 1H), 3.56 (t, J = | | | |
| | 10.2 Hz, 2H), 3.45 (d, J = 25.0 Hz, 4H), | | | |
| | 3.25 (d, J = 6.9 Hz, 2H), 3.11-3.03 (m, 3H), | | | |
| | 2.86-2.68 (m, 4H), 2.37-2.16 (m, 4H), 2.05 | | | |
| | (d, J = 11.0 Hz, 4H), 1.92 (dd, J = 13.7, 9.6 | | | |
| | Hz, 4H), 1.85-1.74 (m, 3H), 1.36-1.27 (m, 3H). | | | |
| A67 | ¹H NMR (400 M Hz, MeOD) δ 7.17 (d, J = | 771.5 | 771.5 | |
| | 8.6 Hz, 2H), 7.12 (d, J = 7.0 Hz, 3H), 7.06 (d, | | | |
| | J = 8.4 Hz, 2H), 6.83 (d, J = 7.7 Hz, 2H), 6.74 | | | |
| | (d, J = 8.5 Hz, 2H), 6.66 (d, J = 8.5 Hz, 2H), | | | |
| | 6.58-6.50 (m, 3H), 4.31 (dd, J = 12.3, 4.9 Hz, | | | |
| | 2H), 3.49-3.35 (m, 4H), 3.09-2.98 (m, 2H), | | | |
| | 2.89-2.65 (m, 4H), 2.58 (d, J = 14.1 Hz, 1H), | | | |
| | 2.41-2.11 (m, 7H), 2.02 (d, J = 3.9 Hz, 2H), | | | |
| | 1.95 (dd, J = 12.6, 5.0 Hz, 1H), 1.91-1.86 (m, | | | |
| | 2H), 1.84-1.72 (m, 3H), 1.60 (d, J = 9.3 Hz, | | | |
| | 1H), 1.34-1.27 (m, 4H). | | | |
| A68 | ¹H NMR (400 MHz, DMSO-d₆) δ ppm 1.67- | 780.4 | 780.4 | |
| | 1.77 (m, 2 H) 1.81-1.82 (m, 1 H) 1.82-2.01 | | | |
| | (m, 2 H) 2.03-2.47 (m, 5 H) 2.45-2.48 (m, | | | |
| | 1 H) 2.72-2.82 (m, 2 H) 2.87-3.07 (m, 6 H) | | | |
| | 3.12-3.22 (m, 1 H) 3.46-3.66 (m, 4 H) 3.70- | | | |
| | 4.02 (m, 6 H) 4.07-4.15 (m, 2 H) 4.26- | | | |
| | 4.42 (m, 2 H) 4.96-5.10 (m, 1 H) 6.06 (d, | | | |
| | J = 8.56 Hz, 2 H) 6.18 (d, J = 8.44 Hz, 2 H) 6.48 | | | |
| | (dd, J = 8.25, 2.38 Hz, 1 H) 6.59-6.66 (m, 2 | | | |
| | H) 6.80-6.86 (m, 2 H) 7.02 (d, J = 8.56 Hz, 1 | | | |
| | H) 7.10-7.21 (m, 4 H) 7.96 (s, 1 H) 9.10 (s, 1 | | | |
| | H) 10.90-10.99 (m, 1 H). | | | |
| A69 | ¹H NMR (400 MHz, DMSO-d₆) δ ppm 1.65- | 780.4 | 780.4 | |
| | 2.14 (m, 4 H) 2.17-2.19 (m, 1 H) 2.34 (br d, | | | |
| | J = 1.22 Hz, 1 H) 3.46-3.67 (m, 7 H) 3.69- | | | |
| | 3.92 (m, 7 H) 3.93-4.01 (m, 2 H) 4.04-4.18 | | | |
| | (m, 4 H) 4.19-4.45 (m, 5 H) 5.04 (br dd, J = | | | |
| | 9.29, 4.65 Hz, 2 H) 6.04-6.12 (m, 2 H) 6.14- | | | |
| | 6.21 (m, 2 H) 6.44-6.51 (m, 1 H) 6.52-6.73 | | | |
| | (m, 3 H) 6.76-6.86 (m, 2 H) 7.02 (br d, J = | | | |
| | 7.09 Hz, 1 H) 7.09-7.23 (m, 5 H) 7.22-7.29 | | | |
| | (m, 1 H) 9.03-9.18 (m, 1 H) 10.87-11.05 | | | |
| | (m, 1 H). | | | |
| A70 | 1H NMR (400 MHz, DMSO) δ 10.97 (s, 1H), | 751.3 | 751.3 | |
| | 9.10 (s, 1H), 7.39 (d, J = 7.6 Hz, 1H), 7.26 (d, | | | |
| | J = 7.6 Hz, 1H), 7.19-7.07 (m, 3H), 6.83 (d, | | | |
| | J = 6.6 Hz, 2H), 6.68-6.58 (m, 2H), 6.56- | | | |
| | 6.43 (m, 3H), 6.20 (d, J = 8.5 Hz, 2H), 5.08 | | | |
| | (dd, J = 13.2, 5.0 Hz, 1H), 4.59-4.46 (m, | | | |
| | 2H), 4.37 (d, J = 16.4 Hz, 1H), 4.27-4.07 | | | |
| | (m, 2H), 3.57-3.47 (m, 3H), 3.07-2.73 (m, | | | |
| | 6H), 2.66-2.55 (m, 1H), 2.44-2.30 (m, | | | |
| | 1H), 2.27-1.52 (m, 15H), 1.27-1.04 (m, 2H). | | | |
| A71 | ¹H NMR (400 M Hz, DMSO-d₆) δ 10.94 (s, | 752.4 | 752.4 | |
| | 1H), 9.10 (s, 1H), 7.20-7.08 (m, 4H), 7.01 | | | |
| | (d, J = 8.4 Hz, 1H), 6.83 (d, J = 6.4 Hz, 2H), | | | |
| | 6.67-6.57 (m, 2H), 6.56-6.43 (m, 3H), | | | |
| | 6.20 (d, J = 8.4 Hz, 2H), 5.03 (dd, J = 13.2, | | | |
| | 5.0 Hz, 1H), 4.40-4.32 (m, 1H), 4.26 (d, J = | | | |
| | 16.8 Hz, 1H), 4.15-4.05 (m, 2H), 4.00- | | | |
| | 3.92 (m, 1H), 3.88-3.75 (m, 1H), 3.58- | | | |
| | 3.44 (m, 2H), 3.29-3.23 (m, 1H), 3.23- | | | |
| | 3.12 (m, 1H), 3.03-2.82 (m, 5H), 2.79- | | | |
| | 2.64 (m, 1H), 2.62-2.54 (m, 1H), 2.47- | | | |
| | 2.28 (m, 3H), 2.25-2.01 (m, 4H), 1.99- | | | |
| | 1.85 (m, 1H), 1.83-1.54 (m, 5H), 1.26- | | | |
| | 1.04 (m, 2H). | | | |
| A72 | ¹H NMR (400 MHz, DMSO-d6) 10.94 (br s, | 780.4 | 780.4 | |
| | 1H), 8.39 (s, 1H), 7.22-7.08 (m, 3H), 7.07- | | | |
| | 7.00 (m, 1H), 6.94 (s, 1H), 6.88-6.77 (m, | | | |
| | 2H), 6.66-6.56 (m, 2H), 6.48 (dd, J = 2.3, | | | |
| | 8.3 Hz, 1H), 6.34-6.15 (m, 2H), 6.13-6.01 | | | |
| | (m, 2H), 5.11-4.97 (m, 1H), 4.32-3.98 (m, | | | |
| | 5H), 3.95-3.70 (m, 5H), 3.62 (br d, J = 7.6 | | | |
| | Hz, 1H), 3.56 (br d, J = 7.5 Hz, 1H), 3.49 (br | | | |
| | s, 1H), 3.26 (br s, 1H), 3.21-3.11 (m, 2H), | | | |
| | 3.05-2.85 (m, 5H), 2.76 (br t, J = 10.2 Hz, | | | |
| | 1H), 2.64-2.52 (m, 2H), 2.38-2.24 (m, 3H), | | | |
| | 2.16-2.04 (m, 2H), 2.00-1.92 (m, 1H), 1.86 | | | |
| | (br dd, J = 7.1, 12.7 Hz, 1H), 1.81-1.63 (m, 2H). | | | |
| A73 | ¹H NMR (400 MHz, DMSO-d6) 10.94 (br s, | 780.4 | 780.4 | |
| | 1H), 8.39 (s, 1H), 7.25-7.09 (m, 3H), 7.05 (s, | | | |
| | 1H), 6.94 (s, 1H), 6.82 (br d, J = 7.1 Hz, 2H), | | | |
| | 6.69-6.56 (m, 2H), 6.48 (br d, J = 8.0 Hz, | | | |
| | 1H), 6.35-6.15 (m, 2H), 6.13-5.97 (m, 2H), | | | |
| | 5.03 (br dd, J = 4.9, 13.1 Hz, 1H), 4.39-4.01 | | | |
| | (m, 5H), 3.96-3.70 (m, 5H), 3.64 (br d, J = | | | |
| | 7.5 Hz, 1H), 3.54 (br d, J = 7.1 Hz, 1H), 3.49 | | | |
| | (br s, 1H), 3.27-3.24 (m, 1H), 3.18 (br s, | | | |
| | 2H), 3.04-2.86 (m, 5H), 2.81-2.72 (m, 1H), | | | |
| | 2.58 (br d, J = 16.5 Hz, 2H), 2.39-2.24 (m, | | | |
| | 3H), 2.19-2.05 (m, 2H), 1.97 (br d, J = 5.4 | | | |
| | Hz, 1H), 1.85 (br dd, J = 6.9, 12.2 Hz, 1H), | | | |
| | 1.69 (br d, J = 10.0 Hz, 2H). | | | |
| A74 | ¹H NMR (400 MHz, DMSO-d6) 10.98 (s, 1H), | 779.3 | 779.3 | |
| | 9.10 (s, 1H), 7.40 (d, J = 7.5 Hz, 1H), 7.27 (d, J = | | | |
| | 7.6 Hz, 1H), 7.19-7.09 (m, 3H), 6.83 (br d, | | | |
| | J = 6.7 Hz, 2H), 6.70-6.57 (m, 2H), 6.48 (dd, | | | |
| | J = 2.4, 8.3 Hz, 1H), 6.18 (d, J = 8.6 Hz, 2H), | | | |
| | 6.06 (d, J = 8.6 Hz, 2H), 5.09 (dd, J = 5.1, | | | |
| | 13.2 Hz, 1H), 4.60-4.49 (m, 2H), 4.38 (d, J = | | | |
| | 17.2 Hz, 1H), 4.22 (d, J = 17.2 Hz, 1H), 4.12 | | | |
| | (d, J = 4.9 Hz, 1H), 3.88 (br t, J = 7.6 Hz, | | | |
| | 1H), 3.82-3.70 (m, 2H), 3.62 (d, J = 7.6 Hz, | | | |
| | 1H), 3.58-3.46 (m, 2H), 3.31-3.24 (m, 2H), | | | |
| | 3.02-2.81 (m, 5H), 2.64-2.53 (m, 2H), 2.45- | | | |
| | 2.34 (m, 2H), 2.27 (br dd, J = 7.4, 12.5 Hz, | | | |
| | 1H), 2.19-1.82 (m, 7H), 1.71 (br d, J = 10.6 | | | |
| | Hz, 3H). | | | |
| A75 | ¹H NMR (400 MHz, DMSO-d6) δ10.98 (s, | 779.3 | 779.3 | |
| | 1H), 9.10 (s, 1H), 7.40 (d, J = 7.6 Hz, 1H), | | | |
| | 7.27 (d, J = 7.6 Hz, 1H), 7.20-7.05 (m, 3H), | | | |
| | 6.83 (br d, J = 6.8 Hz, 2H), 6.69-6.58 (m, | | | |
| | 2H), 6.48 (dd, J = 2.4, 8.3 Hz, 1H), 6.18 (d, | | | |
| | J = 8.5 Hz, 2H), 6.06 (d, J = 8.6 Hz, 2H), 5.09 | | | |
| | (dd, J = 4.9, 13.4 Hz, 1H), 4.57-4.48 (m, 2H), | | | |
| | 4.38 (d, J = 17.2 Hz, 1H), 4.22 (d, J = 17.2 Hz, | | | |
| | 1H), 4.12 (br d, J = 4.9 Hz, 1H), 3.87 (t, J = 7.5 | | | |
| | Hz, 1H), 3.75 (dd, J = 7.7, 16.0 Hz, 2H), 3.64 | | | |
| | (d, J = 7.6 Hz, 1H), 3.58-3.45 (m, 2H), 3.26 | | | |
| | (br s, 1H), 3.06-2.82 (m, 5H), 2.64-2.53 (m, | | | |
| | 2H), 2.46-2.21 (m, 4H), 2.15-1.82 (m, 7H), | | | |
| | 1.70 (br d, J = 9.1 Hz, 3H). | | | |
| A76 | ¹H NMR (400 MHz, DMSO-d6) δ 11.01 (s, | 807.4 | 807.4 | |
| | 1H), 9.13 (br s, 1H), 8.32 (d, J = 5.1 Hz, 1H), | | | |
| | 8.17 (s, 1H), 7.30 (d, J = 5.1 Hz, 1H), 7.19- | | | |
| | 7.07 (m, 3H), 6.82 (br d, J = 6.4 Hz, 2H), 6.68- | | | |
| | 6.58 (m, 2H), 6.56-6.44 (m, 3H), 6.20 (d, J = | | | |
| | 8.6 Hz, 2H), 5.14 (br dd, J = 5.0, 13.2 Hz, | | | |
| | 2H), 4.49-4.39 (m, 1H), 4.33-4.24 (m, 1H), | | | |
| | 4.12 (br d, J = 4.8 Hz, 1H), 3.85 (br t, J = 7.8 | | | |
| | Hz, 1H), 3.21 (br s, 2H), 3.08-2.85 (m, 6H), | | | |
| | 2.77-2.53 (m, 4H), 2.49-2.40 (m, 2H), 2.36 - | | | |
| | 2.17 (m, 4H), 2.14-2.06 (m, 1H), 2.04- | | | |
| | 1.84 (m, 4H), 1.75-1.49 (m, 7H), 1.31 (br | | | |
| | dd, J = 8.1, 12.3 Hz, 1H) | | | |
| A77 | ¹H NMR (400 MHz, DMSO-d₆) δ ppm 1.34 | 807.4 | 807.4 | |
| | (br dd, J = 12.44, 8.19 Hz, 1 H) 1.54-1.77 (m, | | | |
| | 7 H) 1.85-2.13 (m, 7 H) 2.30-2.44 (m, 3 H) | | | |
| | 2.75-3.15 (m, 10 H) 3.21-3.58 (m, 6 H) | | | |
| | 3.87 (t, J = 7.69 Hz, 1 H) 4.13 (d, J = 4.88 Hz, 1 | | | |
| | H) 4.16-4.27 (m, 1 H) 4.34 (d, J = 17.01 Hz, 1 | | | |
| | H) 4.42-4.51 (m, 2 H) 5.08 (dd, J = 13.19, | | | |
| | 5.07 Hz, 1 H) 6.21 (d, J = 8.63 Hz, 2 H) 6.47- | | | |
| | 6.57 (m, 3 H) 6.59-6.68 (m, 2 H) 6.83 (br d, | | | |
| | J = 6.50 Hz, 2 H) 7.01 (s, 1 H) 7.09-7.18 (m, | | | |
| | 3 H) 7.46 (s, 1 H) 8.15 (s, 1 H) 9.09 (br s, 1 | | | |
| | H) 10.89-11.04 (m, 1 H) | | | |
| A78 | ¹H NMR (400 M Hz, DMSO-d₆) δ 10.94 (s, | 768.4 | 768.4 | |
| | 1H), 9.07 (s, 1H), 7.19-7.07 (m, 4H), 7.01 | | | |
| | (d, J = 8.4 Hz, 1H), 6.82 (d, J = 6.8 Hz, 2H), | | | |
| | 6.66-6.56 (m, 2H), 6.50-6.44 (m, 1H), | | | |
| | 6.17 (d, J = 8.4 Hz, 2H), 6.05 (d, J = 8.4 Hz, | | | |
| | 2H), 5.03 (dd, J = 13.4, 4.9 Hz, 1H), 4.40- | | | |
| | 4.21 (m, 3H), 4.15-4.06 (m, 2H), 4.01- | | | |
| | 3.75 (m, 4H), 3.50-3.39 (m, 4H), 3.20- | | | |
| | 3.06 (m, 2H), 3.02-2.84 (m, 5H), 2.79- | | | |
| | 2.54 (m, 2H), 2.39-2.29 (m, 3H), 2.15- | | | |
| | 1.88 (m, 4H), 1.77-1.59 (m, 4H). | | | |
| A79 | ¹H NMR (400 M Hz, DMSO-d₆) δ 10.93 (s, | 754.4 | 754.4 | |
| | 1H), 8.17 (s, 1H), 7.18-7.10 (m, 4H), 7.01 | | | |
| | (d, J = 8.6 Hz, 1H), 6.83 (d, J = 6.6 Hz, 2H), | | | |
| | 6.65-6.57 (m, 2H), 6.53-6.46 (m, 3H), | | | |
| | 6.26 (d, J = 8.6 Hz, 2H), 5.06-4.99 (m, 1H), | | | |
| | 4.43-4.33 (m, 1H), 4.21 (d, J = 11.2 Hz, | | | |
| | 1H), 4.10 (d, J = 12.4 Hz, 1H), 3.98-3.91 | | | |
| | (m, 1H), 3.87-3.75 (m, 3H), 2.92 (s, 2H), | | | |
| | 2.80-2.70 (m, 3H), 2.42-2.35 (m, 1H), | | | |
| | 2.09-1.89 (m, 3H), 1.74-1.59 (m, 2H), | | | |
| | 1.24 (s, 2H). | | | |
| A80 | ¹H NMR (400 M Hz, DMSO-d₆) δ 10.97 (s, | 753.4 | 753.4 | |
| | 1H), 8.36 (s, 2H), 7.40 (d, J = 7.6 Hz, 1H), | | | |
| | 7.26 (d, J = 7.6 Hz, 1H), 7.21-7.08 (m, 4H), | | | |
| | 6.83 (d, J = 7.0 Hz, 3H), 6.67-6.59 (m, 3H), | | | |
| | 6.53-6.44 (m, 4H), 6.26 (d, J = 8.6 Hz, 3H), | | | |
| | 5.14-5.03 (m, 1H), 4.54-4.46 (m, 2H), | | | |
| | 4.37 (d, J = 12.0 Hz, 1H), 4.23-4.08 (m, | | | |
| | 3H), 2.99-2.90 (m, 4H), 2.64 (d, J = 14.2 | | | |
| | Hz, 4H), 2.10-1.92 (m, 5H), 1.81 (d, J = | | | |
| | 12.0 Hz, 2H), 1.73-1.62 (m, 4H). | | | |
| A81 | ¹H NMR (400 M Hz, DMSO-d₆) δ 10.97 (s, | 793.4 | 793.4 | |
| | 1H), 8.19 (s, 1H), 7.38 (d, J = 7.6 Hz, 1H), | | | |
| | 7.26 (d, J = 7.6 Hz, 1H), 7.15-7.04 (m, 3H), | | | |
| | 6.84-6.75 (m, 2H), 6.65-6.56 (m, 2H), | | | |
| | 6.52-6.39 (m, 3H), 6.18 (d, J = 8.4 Hz, 2H), | | | |
| | 5.12-5.04 (m, 1H), 4.54-4.44 (m, 2H), | | | |
| | 4.41-4.31 (m, 1H), 4.24-4.16 (m, 1H), | | | |
| | 4.08 (d, J = 4.8 Hz, 1H), 3.84 (s, 2H), 3.55- | | | |
| | 3.48 (m, 4H), 3.27-3.14 (m, 2H), 2.96- | | | |
| | 2.85 (m, 3H), 2.69-2.55 (m, 3H), 2.45- | | | |
| | 2.30 (m, 1H), 2.19-2.04 (m, 5H), 2.00- | | | |
| | 1.93 (m, 1H), 1.87 (d, J = 8.6 Hz, 3H), 1.69- | | | |
| | 1.57 (m, 3H), 1.49-1.37 (m, 1H), 1.29- | | | |
| | 1.17 (m, 2H). | | | |
| A82 | ¹H NMR (400 M Hz, DMSO-d₆) δ 10.92 (s, | 782.5 | 782.5 | |
| | 1H), 9.04 (S, 1H), 7.17-7.11 (m, 4H), 6.99 | | | |
| | (d, J = 8.4 Hz, 1H), 6.83 (d, J = 7.2 Hz, 2H), | | | |
| | 6.63-6.50 (m, 5H), 6.20 (d, J = 8.4 Hz, 2H), | | | |
| | 5.02 (dd, J = 13.2, 5.2 Hz, 1H), 4.29 (dd, J = | | | |
| | 29.6, 13.6 Hz, 2H), 4.15-3.91 (m, 4H), 3.78 | | | |
| | (d, J = 11.6 Hz, 1H), 3.20 (M, 4H), 3.12 (s, | | | |
| | 3H), 2.88 (M, 6H), 2.59 (M, 1H), 2.42-2.30 | | | |
| | (m, 4H), 2.10 (M, 1H), 1.95 (M, 2H), 1.68 | | | |
| | (M, 6H). | | | |
| A83 | 1HNMR (400 MHz, DMSO) δ 10.93 (s, 1H), | 794.8 | 794.8 | |
| | 9.10 (s, 1H), 7.21-7.08 (m, 4H), 7.00 (d, J = | | | |
| | 8.2 Hz, 1H), 6.82 (d, J = 5.4 Hz, 2H), 6.69- | | | |
| | 6.58 (m, 2H), 6.50 (t, J = 8.0 Hz, 3H), 6.20 (d, | | | |
| | J = 8.2 Hz, 2H), 5.08-4.99 (m, 1H), 4.31- | | | |
| | 4.19 (m, 2H), 4.16-4.06 (m, 2H), 3.96- | | | |
| | 3.88 (m, 1H), 3.88-3.74 (m, 3H), 3.52 (s, | | | |
| | 4H), 3.19-3.07 (m, 1H), 3.05-2.88 (m, | | | |
| | 3H), 2.77-2.65 (m, 3H), 2.63-2.55 (m, | | | |
| | 1H), 2.44-2.31 (m, 1H), 2.23-2.06 (m, | | | |
| | 5H), 2.03-1.88 (m, 2H), 1.78-1.63 (m, | | | |
| | 2H), 1.54-1.42 (m, 1H), 1.28-1.15 (m, 3H). | | | |
| A84 | 1H NMR (400 MHz, DMSO) δ 10.78 (s, 1H), | 731.4 | 731.4 | |
| | 8.16 (s, 1H), 7.07 (d, J = 6.9 Hz, 3H), 6.98 (t, | | | |
| | J = 8.8 Hz, 1H), 6.73 (d, J = 8.1 Hz, 2H), 6.57- | | | |
| | 6.54 (m, 2H), 6.44 (dd, J = 12.2, 6.1 Hz, | | | |
| | 3H), 6.36 (d, J = 8.5 Hz, 1H), 6.32-6.27 (m, | | | |
| | 1H), 6.11 (s, 1H), 5.99 (d, J = 7.6 Hz, 1H), | | | |
| | 4.64 (d, J = 4.9 Hz, 1H), 4.29 (q, J = 12.2 Hz, | | | |
| | 1H), 3.55 (dd, J = 15.6, 9.2 Hz, 2H), 3.21 (dd, | | | |
| | J = 17.2, 5.9 Hz, 2H), 2.94 (s, 3H), 2.89 (d, J = | | | |
| | 7.2 Hz, 1H), 2.75-2.67 (m, 1H), 2.61- | | | |
| | 2.54 (m, 4H), 2.19 (d, J = 6.8 Hz, 3H), 2.08 | | | |
| | (dd, J = 13.9, 7.1 Hz, 1H), 1.96 (d, J = 15.9 | | | |
| | Hz, 2H), 1.91-1.84 (m, 1H), 1.75 (dd, J = | | | |
| | 13.7, 6.9 Hz, 2H), 1.62 (dd, J = 16.6, 8.9 Hz, | | | |
| | 6H), 1.26-1.15 (m, 4H). | | | |
| A85 | 1H NMR (400 MHz, DMSO) δ 10.78 (s, 1H), | 731.4 | 731.4 | |
| | 8.18 (s, 1H), 7.07 (d, J = 7.0 Hz, 3H), 6.99 (t, | | | |
| | J = 8.8 Hz, 1H), 6.73 (d, J = 7.7 Hz, 2H), 6.55 | | | |
| | (d, J = 8.6 Hz, 2H), 6.46-6.41 (m, 3H), 6.36 | | | |
| | (d, J = 8.4 Hz, 1H), 6.30 (dd, J = 8.7, 1.8 Hz, | | | |
| | 1H), 6.11 (d, J = 1.8 Hz, 1H), 5.99 (d, J = 7.8 | | | |
| | Hz, 1H), 4.64 (d, J = 5.0 Hz, 1H), 4.36-4.26 | | | |
| | (m, 1H), 3.60-3.45 (m, 3H), 3.24-3.17 (m, | | | |
| | 2H), 2.94 (s, 3H), 2.87 (s, 1H), 2.74-2.67 | | | |
| | (m, 1H), 2.59-2.53 (m, 4H), 2.37 (d, J = | | | |
| | 36.9 Hz, 1H), 2.17 (d, J = 7.3 Hz, 2H), 2.08 | | | |
| | (dd, J = 13.8, 3.5 Hz, 1H), 1.97 (d, J = 15.2 | | | |
| | Hz, 2H), 1.89-1.84 (m, 1H), 1.77-1.71 (m, | | | |
| | 2H), 1.65-1.57 (m, 6H), 1.25-1.12 (m, 3H). | | | |
| A86 | 1H NMR (400 MHz, MeOD) δ 7.06 (d, J = | 713.4 | 713.4 | |
| | 8.6 Hz, 5H), 6.77-6.73 (m, 4H), 6.70 (d, J = | | | |
| | 9.6 Hz, 2H), 6.64 (d, J = 2.5 Hz, 1H), 6.59 (d, | | | |
| | J = 8.5 Hz, 1H), 6.54-6.47 (m, 2H), 4.27 | | | |
| | (dd, J = 11.8, 4.8 Hz, 1H), 3.69 (t, J = 14.9 | | | |
| | Hz, 4H), 3.22 (d, J = 13.2 Hz, 2H), 3.13 (dd, J = | | | |
| | 13.4, 5.1 Hz, 4H), 3.09 (s, 3H), 3.05-2.95 | | | |
| | (m, 2H), 2.84-2.68 (m, 3H), 2.36-2.20 (m, | | | |
| | 3H), 2.07 (d, J = 12.7 Hz, 4H), 2.01-1.87 | | | |
| | (m, 3H), 1.75-1.62 (m, 3H), 1.29 (s, 2H). | | | |
| A87 | 1H NMR (400 MHz, MeOD) δ 7.07-7.02 | 713.4 | 713.4 | |
| | (m, 5H), 6.73 (t, J = 7.1 Hz, 4H), 6.61 (dd, J = | | | |
| | 5.3, 3.0 Hz, 2H), 6.49 (dd, J = 13.2, 5.3 Hz, | | | |
| | 2H), 6.41 (dd, J = 8.5, 2.3 Hz, 1H), 6.18 (d, J = | | | |
| | 1.9 Hz, 1H), 4.76 (d, J = 5.4 Hz, 1H), 4.27 | | | |
| | (dd, J = 11.2, 4.3 Hz, 1H), 3.62 (d, J = 12.6 | | | |
| | Hz, 4H), 3.29-3.19 (m, 2H), 3.16-3.03 (m, | | | |
| | 2H), 3.01 (s, 3H), 2.98 (d, J = 5.2 Hz, 2H), | | | |
| | 2.77 (dd, J = 11.1, 5.8 Hz, 2H), 2.67 (d, J = | | | |
| | 13.1 Hz, 2H), 2.36-2.27 (m, 2H), 2.03- | | | |
| | 1.86 (m, 7H), 1.69-1.61 (m, 1H), 1.51- | | | |
| | 1.41 (m, 2H), 1.31 (s, 3H). | | | |
| A88 | 1H NMR (400 MHz, MeOD) δ 7.12 (d, J = | 761.4 | 761.4 | |
| | 8.6 Hz, 2H), 7.03 (dd, J = 5.1, 1.8 Hz, 3H), | | | |
| | 6.72 (dd, J = 8.7, 6.3 Hz, 4H), 6.64-6.59 (m, | | | |
| | 2H), 6.51-6.46 (m, 2H), 6.40 (dd, J = 8.4, | | | |
| | 2.3 Hz, 1H), 6.18 (d, J = 2.2 Hz, 1H), 4.76 (d, | | | |
| | J = 5.3 Hz, 1H), 4.28 (dd, J = 11.9, 4.9 Hz, | | | |
| | 1H), 3.56 (d, J = 12.2 Hz, 2H), 3.21 (dt, J = | | | |
| | 13.4, 8.6 Hz, 2H), 3.00 (s, 3H), 2.96 (dd, J = | | | |
| | 11.4, 5.9 Hz, 2H), 2.84-2.76 (m, 1H), 2.74- | | | |
| | 2.65 (m, 2H), 2.60 (dd, J = 9.2, 2.6 Hz, 2H), | | | |
| | 2.41-2.17 (m, 6H), 2.04-1.94 (m, 2H), | | | |
| | 1.88 (dd, J = 21.4, 9.0 Hz, 2H), 1.66 (dt, J = | | | |
| | 17.7, 11.4 Hz, 2H), 1.32 (dd, J = 16.9, 6.0 Hz, 4H). | | | |
| A89 | 1H NMR (400 MHz, MeOD) δ 7.19 (d, J = | 761.4 | 761.4 | |
| | 8.5 Hz, 2H), 7.05 (dd, J = 5.0, 1.7 Hz, 3H), | | | |
| | 6.74 (dd, J = 9.2, 5.2 Hz, 4H), 6.64 (dd, J = | | | |
| | 9.2, 2.1 Hz, 3H), 6.59 (d, J = 8.4 Hz, 1H), | | | |
| | 6.49 (dd, J = 8.2, 2.5 Hz, 1H), 6.42 (d, J = 4.4 | | | |
| | Hz, 1H), 4.82 (d, J = 6.0 Hz, 2H), 4.35-4.27 | | | |
| | (m, 1H), 3.65 (d, J = 13.1 Hz, 2H), 3.19- | | | |
| | 3.09 (m, 4H), 3.07 (s, 3H), 2.98 (dd, J = 12.7, | | | |
| | 6.6 Hz, 3H), 2.82-2.71 (m, 2H), 2.61 (dd, J = | | | |
| | 18.2, 8.0 Hz, 1H), 2.45-2.27 (m, 4H), 2.20- | | | |
| | 2.14 (m, 1H), 2.03-1.91 (m, 3H), 1.68- | | | |
| | 1.58 (m, 3H), 1.30 (d, J = 8.1 Hz, 3H). | | | |
| A90 | 1H NMR (400 MHz, DMSO) δ 10.79 (s, 1H), | 761.4 | 761.4 | |
| | 9.07 (s, 1H), 7.09 (t, J = 16.3 Hz, 5H), 6.70 | | | |
| | (dd, J = 26.6, 5.6 Hz, 4H), 6.60-6.53 (m, | | | |
| | 2H), 6.45 (d, J = 9.4 Hz, 1H), 6.34 (dd, J = | | | |
| | 24.7, 8.3 Hz, 2H), 6.12 (s, 1H), 6.02-5.87 | | | |
| | (m, 1H), 4.65 (d, J = 5.1 Hz, 1H), 4.33 (d, J = | | | |
| | 12.9 Hz, 1H), 4.02 (t, J = 19.2 Hz, 1H), 3.59 | | | |
| | (t, J = 12.6 Hz, 2H), 3.22 (d, J = 12.7 Hz, 2H), | | | |
| | 3.13-3.05 (m, 3H), 2.95 (s, 3H), 2.87 (d, J = | | | |
| | 6.7 Hz, 1H), 2.78-2.60 (m, 4H), 2.35-2.24 | | | |
| | (m, 3H), 2.20 (dd, J = 12.5, 8.7 Hz, 2H), 2.14- | | | |
| | 2.04 (m, 2H), 1.97-1.86 (m, 2H), 1.82- | | | |
| | 1.74 (m, 2H), 1.59 (d, J = 9.4 Hz, 1H), 1.33 | | | |
| | 1.24 (m, 2H). | | | |
| A91 | 1H NMR (400 MHz, DMSO) δ 10.92 (s, 1H), | 800.4 | 800.4 | |
| | 8.34 (s, 1H), 7.11-7.02 (m, 4H), 6.93 (s, | | | |
| | 1H), 6.76 (d, J = 7.8 Hz, 2H), 6.60-6.52 (m, | | | |
| | 2H), 6.50-6.43 (m, 1H), 6.22-6.14 (m, | | | |
| | 2H), 5.09-4.98 (m, 1H), 4.67 (d, J = 5.0 Hz, | | | |
| | 1H), 4.33-4.15 (m, 3H), 3.93-3.81 (m, | | | |
| | 2H), 3.29-3.22 (m, 4H), 2.98-2.88 (m, | | | |
| | 8H), 2.80-2.72 (m, 1H), 2.64-2.56 (m, | | | |
| | 3H), 2.38-2.25 (m, 2H), 2.23-2.05 (m, | | | |
| | 4H), 1.98-1.92 (m, 1H), 1.77-1.60 (m, | | | |
| | 4H), 1.29-1.20 (m, 2H). | | | |
| A92 | 1H NMR (400 MHz, DMSO) δ 10.79 (s, 1H), | 761.4 | 761.4 | |
| | 9.06 (s, 1H), 7.16 (d, J = 7.9 Hz, 2H), 7.07 (d, | | | |
| | J = 6.6 Hz, 3H), 6.76-6.70 (m, 2H), 6.67 (d, | | | |
| | J = 8.4 Hz, 2H), 6.56 (dd, J = 10.3, 5.2 Hz, | | | |
| | 2H), 6.45 (dd, J = 9.3, 3.2 Hz, 1H), 6.41- | | | |
| | 6.36 (m, 1H), 6.32 (dd, J = 9.4, 3.9 Hz, 1H), | | | |
| | 6.14 (d, J = 1.2 Hz, 1H), 5.95 (d, J = 7.3 Hz, | | | |
| | 1H), 4.66 (t, J = 4.4 Hz, 1H), 4.38-4.28 (m, | | | |
| | 1H), 4.03 (t, J = 11.8 Hz, 1H), 3.67-3.50 (m, | | | |
| | 3H), 3.25-3.16 (m, 2H), 3.14-3.00 (m, | | | |
| | 3H), 2.95 (s, 3H), 2.93-2.78 (m, 2H), 2.78- | | | |
| | 2.64 (m, 3H), 2.63-2.56 (m, 3H), 2.24- | | | |
| | 2.06 (m, 3H), 1.99-1.85 (m, 2H), 1.83- | | | |
| | 1.74 (m, 2H), 1.64-1.56 (m, 1H), 1.27 (dd, J = | | | |
| | 19.9, 7.8 Hz, 2H). | | | |
| A93 | 1H NMR (400 MHz, DMSO) δ 10.97 (s, 1H), | 799.4 | 799.4 | |
| | 8.20 (s, 1H), 7.40 (d, J = 7.6 Hz, 1H), 7.26 (d, | | | |
| | J = 7.6 Hz, 1H), 7.13-7.05 (m, 3H), 6.76 (d, | | | |
| | J = 6.2 Hz, 2H), 6.62-6.54 (m, 2H), 6.49- | | | |
| | 6.44 (m, 1H), 6.23-6.15 (m, 2H), 5.12- | | | |
| | 4.98 (m, 1H), 4.67 (d, J = 5.4 Hz, 1H), 4.51 (t, | | | |
| | J = 10.0 Hz, 2H), 4.40-4.33 (m, 1H), 4.26- | | | |
| | 4.16 (m, 1H), 3.00-2.81 (m, 9H), 2.68- | | | |
| | 2.54 (m, 3H), 2.47-2.31 (m, 2H), 2.24- | | | |
| | 2.14 (m, 3H), 2.05-1.88 (m, 5H), 1.82- | | | |
| | 1.74 (m, 2H), 1.70-1.54 (m, 4H), 1.31- | | | |
| | 1.17 (m, 2H). | | | |
| A94 | 1H NMR (400 MHz, DMSO) δ 10.84 (s, 1H), | 789.4 | 789.4 | |
| | 8.50 (d, J = 8.2 Hz, 1H), 7.57 (d, J = 8.2 Hz, | | | |
| | 1H), 7.31 (d, J = 8.4 Hz, 1H), 7.09 (d, J = 6.6 | | | |
| | Hz, 3H), 6.76 (d, J = 6.2 Hz, 2H), 6.61-6.53 | | | |
| | (m, 2H), 6.49 (d, J = 8.2 Hz, 1H), 6.25-6.13 | | | |
| | (m, 2H), 4.81-4.63 (m, 2H), 4.44 (d, J = 8.4 | | | |
| | Hz, 1H), 4.13-3.98 (m, 1H), 3.84-3.76 (m, | | | |
| | 1H), 3.26-3.20 (m, 4H), 2.99-2.93 (m, | | | |
| | 6H), 2.81-2.72 (m, 2H), 2.65-2.54 (m, | | | |
| | 2H), 2.30-2.07 (m, 6H), 2.02-1.95 (m, | | | |
| | 1H), 1.83-1.59 (m, 6H), 1.32-1.18 (m, 3H). | | | |
| A95 | ¹H NMR (400 MHz, DMSO) δ 10.97 (s, 1H), | 821.4 | 821.9 | |
| | 9.05 (s, 1H), 8.14 (s, 1H), 7.37 (d, J = 7.6 Hz, | | | |
| | 1H), 7.28 (d, J = 7.6 Hz, 1H), 7.07 (d, J = 6.8 | | | |
| | Hz, 3H), 6.85-6.69 (m, 2H), 6.63-6.53 (m, | | | |
| | 2H), 6.45 (dd, J = 8.3, 2.2 Hz, 1H), 6.36 (d, J = | | | |
| | 8.4 Hz, 1H), 6.30 (d, J = 10.0 Hz, 1H), 6.11 | | | |
| | (s, 1H), 5.08 (dd, J = 13.2, 5.0 Hz, 1H), 4.64 | | | |
| | (d, J = 5.1 Hz, 1H), 4.55 (s, 2H), 4.38 (d, J = | | | |
| | 17.2 Hz, 1H), 4.21 (d, J = 17.0 Hz, 1H), 3.20 | | | |
| | (dd, J = 12.7, 4.5 Hz, 3H), 3.00 (s, 4H), 2.94 | | | |
| | (s, 4H), 2.92 (s, 3H), 2.84 (s, 1H), 2.67 (s, | | | |
| | 1H), 2.62 (s, 1H), 2.57 (s, 1H), 2.43 (d, J = | | | |
| | 4.8 Hz, 1H), 2.41-2.37 (m, 1H), 2.34 (d, J = | | | |
| | 14.2 Hz, 1H), 1.97 (d, J = 11.6 Hz, 6H), 1.79- | | | |
| | 1.69 (m, 2H), 1.65 (s, 2H), 1.59 (s, 1H), | | | |
| | 1.53 (s, 1H), 1.50-1.37 (m, 2H) | | | |
| A96 | 1H NMR (400 M Hz, DMSO-d6) δ 7.08-7.02 | 753.4 | 753.4 | |
| | (m, 5H), 6.79-6.74 (m, 4H), 6.72-6.70 (m, | | | |
| | 2H), 6.63 (s, 1H), 6.59-6.57 (m, 1H), 6.49- | | | |
| | 6.47 (m, 2H), 4.28-4.24 (m, 1H), 3.58-3.55 | | | |
| | (m, 2H), 3.48-3.46 (m, 1H), 3.26-3.24 (m, | | | |
| | 4H), 3.13-3.12 (m, 2H), 3.08-3.06 (m, 3H), | | | |
| | 3.05-2.99 (m, 3H), 2.81-2.73 (m, 4H), 2.32- | | | |
| | 2.22 (m, 4H), 2.07-2.00 (m, 2H), 1.95-1.87 | | | |
| | (m, 4H), 1.85-1.76 (m, 4H), 1.72-1.66 (m, 4H) | | | |
| A97 | 1H NMR (400 MHz, DMSO) δ 10.93 (s, 1H), | 800.4 | 800.4 | |
| | 7.17 (d, J = 8.2 Hz, 1H), 7.14-7.07 (m, 3H), | | | |
| | 7.01 (d, J = 8.6 Hz, 1H), 6.87-6.81 (m, 2H), | | | |
| | 6.69-6.58 (m, 2H), 6.55-6.47 (m, 1H), | | | |
| | 6.26 (d, J = 12.0 Hz, 1H), 6.06 (d, J = 7.0 Hz, | | | |
| | 1H), 5.07-4.98 (m, 1H), 4.45 (d, J = 5.2 Hz, | | | |
| | 1H), 4.40-4.33 (m, 1H), 4.30-4.22 (m, | | | |
| | 1H), 4.14-4.07 (m, 1H), 4.02-3.94 (m, | | | |
| | 1H), 3.86-3.78 (m, 1H), 3.47 (s, 3H), 3.28- | | | |
| | 3.16 (m, 4H), 3.04-2.88 (m, 5H), 2.77- | | | |
| | 2.65 (m, 1H), 2.62-2.55 (m, 1H), 2.46- | | | |
| | 2.28 (m, 4H), 2.27-2.16 (m, 3H), 2.14- | | | |
| | 2.05 (m, 1H), 1.98-1.92 (m, 1H), 1.80- | | | |
| | 1.69 (m, 4H), 1.65-1.56 (m, 1H), 1.29- | | | |
| | 1.14 (m, 2H).. | | | |
| A98 | 1H NMR (400 MHz, DMSO) δ 10.93 (s, 1H), | 800.4 | 800.4 | |
| | 7.17 (d, J = 8.2 Hz, 1H), 7.13-7.07 (m, 3H), | | | |
| | 7.04-6.98 (m, 1H), 6.91-6.82 (m, 2H), | | | |
| | 6.67-6.58 (m, 2H), 6.53-6.46 (m, 1H), | | | |
| | 6.29-6.22 (m, 1H), 6.06 (d, J = 7.0 Hz, 1H), | | | |
| | 5.08-4.98 (m, 1H), 4.45 (d, J = 5.2 Hz, 1H), | | | |
| | 4.40-4.33 (m, 1H), 4.29-4.21 (m, 1H), | | | |
| | 4.16-4.05 (m, 1H), 4.00-3.94 (m, 1H), | | | |
| | 3.86-3.76 (m, 1H), 3.47 (s, 3H), 3.36-3.28 | | | |
| | (m, 4H), 3.04-2.81 (m, 6H), 2.77-2.66 (m, | | | |
| | 1H), 2.61-2.54 (m, 1H), 2.46-2.33 (m, | | | |
| | 3H), 2.28-2.16 (m, 3H), 2.13-2.06 (m, | | | |
| | 1H), 1.98-1.91 (m, 1H), 1.80-1.69 (m, | | | |
| | 4H), 1.64-1.52 (m, 1H), 1.28-1.13 (m, 2H). | | | |
| A99 | 1H NMR (400 MHz, DMSO) δ 10.93 (s, 1H), | 840.4 | 840.4 | |
| | 7.21-6.98 (m, 5H), 6.75 (d, J = 6.4 Hz, 2H), | | | |
| | 6.63-6.45 (m, 3H), 6.18 (dd, J = 10.4, 6.8 | | | |
| | Hz, 2H), 5.03 (dd, J = 13.2, 4.8 Hz, 1H), 4.67 | | | |
| | (d, J = 4.8 Hz, 1H), 4.30 (dd, J = 34.4, 13.6 | | | |
| | Hz, 2H), 4.10 (d, J = 16.8 Hz, 1H), 3.95 (dd, J = | | | |
| | 8.4, 6.8 Hz, 1H), 3.80 (d, J = 11.2 Hz, 1H), | | | |
| | 3.25-3.03 (m, 4H), 2.95 (s, 3H), 2.92-2.69 | | | |
| | (m, 9H), 2.57 (d, J = 16.8 Hz, 1H), 2.43- | | | |
| | 1.92 (m, 8H), 1.77-1.40 (m, 8H). | | | |
| A100 | ¹H NMR (400 MHz, DMSO-d₆) δ 10.97 (s, 1 | 839.4 | 839.4 | |
| | H), 9.13 (br s, 1 H), 7.22-7.43 (m, 2 H), 7.00- | | | |
| | 7.15 (m, 3 H), 6.68-6.81 (m, 2 H), 6.41- | | | |
| | 6.62 (m, 3 H), 6.11-6.24 (m, 2 H), 5.08 (dd, | | | |
| | J = 13.32, 4.94 Hz, 1 H), 4.66 (br d, J = 5.00 | | | |
| | Hz, 1 H), 4.52 (s, 2 H), 4.35-4.36 (m, 1 H), | | | |
| | 4.37 (d, J = 17.13 Hz, 1 H), 4.21 (d, J = 17.13 | | | |
| | Hz, 1 H), 3.24 (br s, 1 H), 2.72-2.95 (m, 12 | | | |
| | H), 2.55-2.64 (m, 2 H), 2.32-2.46 (m, 2 H), | | | |
| | 2.05-2.21 (m, 3 H), 1.83-2.03 (m, 6 H), | | | |
| | 1.51-1.77 (m, 7 H), 1.44 (br dd, J = 9.26, | | | |
| | 5.63 Hz, 2 H). | | | |
| A101 | 1H NMR (400 MHz, DMSO) δ 10.97 (s, 1H), | 840.4 | 840.4 | |
| | 8.20 (s, 1H), 7.40 (d, J = 7.6 Hz, 1H), 7.26 | | | |
| | (d, J = 7.6 Hz, 1H), 7.11-7.07 (m, 3H), 6.77 | | | |
| | (d, J = 7.2 Hz, 2H), 6.59-6.46 (m, 3H), 6.22- | | | |
| | 6.18 (m, 2H), 5.08 (dd, J = 13.2, 5.2 Hz, 1H), | | | |
| | 4.67 (d, J = 5.6 Hz, 1H), 4.55-4.50 (m, 2H), | | | |
| | 4.09-4.06 (m, 1H), 4.29(dd, J = 68.4, 17.2 Hz, | | | |
| | 2H), 3.23-2.82 (m, 9H), 2.67-2.53 (m, 3H), | | | |
| | 2.44-2.14 (m, 5H), 2.00-1.62 (m, 11H), | | | |
| | 1.25-1.22 (m, 2H). | | | |
| A102 | 1H NMR (400 MHz, DMSO) δ 10.93 (s, 1H), | 840.4 | 840.4 | |
| | 9.14 (s, 1H), 7.17 (d, J = 8.4 Hz, 1H), 7.13- | | | |
| | 7.05 (m, 3H), 7.00 (d, J = 8.4 Hz, 1H), 6.88- | | | |
| | 6.78 (m, 2H), 6.68-6.58 (m, 2H), 6.54- | | | |
| | 6.45 (m, 1H), 6.24 (d, J = 11.6Hz, 1H), 6.05 | | | |
| | (d, J = 7.2 Hz, 1H), 5.03 (dd, J = 13.2, 5.2 Hz, | | | |
| | 1H), 4.44 (d, J = 5.2 Hz, 1H), 4.38-4.31 (m, | | | |
| | 1H), 4.26 (d, J = 16.8 Hz, 1H), 4.10 (d, J = | | | |
| | 16.8 Hz, 1H), 4.00-3.90 (m, 1H), 3.80 (d, J = | | | |
| | 11.6 Hz, 1H), 3.34-3.31 (m, 1H), 3.18- | | | |
| | 3.11 (m, 1H), 3.06-2.54 (m, 12H), 2.47- | | | |
| | 2.36 (m, 3H), 2.28-2.07 (m, 2H), 2.00- | | | |
| | 1.87 (m, 3H), 1.73 (t, J = 10.4 Hz, 2H), 1.68- | | | |
| | 1.60 (m, 2H), 1.57-1.48 (m, 2H), 1.46- | | | |
| | 1.35 (m, 2H). | | | |
| A103 | 1H NMR (400 MHz, DMSO) δ 10.92 (s, 1H), | 840.4 | 840.4 | |
| | 9.13 (s, 1H), 7.17 (d, J = 8.2 Hz, 1H), 7.13- | | | |
| | 7.05 (m, 3H), 7.00 (d, J = 8.4 Hz, 1H), 6.88- | | | |
| | 6.77 (m, 2H), 6.68-6.57 (m, 2H), 6.49 (dd, J = | | | |
| | 8.2, 2.4 Hz, 1H), 6.24 (d, J = 11.6 Hz, 1H), | | | |
| | 6.05 (d, J = 7.2 Hz, 1H), 5.03 (dd, J = 13.2, | | | |
| | 4.8 Hz, 1H), 4.44 (d, J = 5.2 Hz, 1H), 4.34 | | | |
| | (dd, J = 10.8, 2.4 Hz, 1H), 4.26 (d, J = 16.8 | | | |
| | Hz, 1H), 4.09 (d, J = 16.8 Hz, 1H), 4.01- | | | |
| | 3.89 (m, 1H), 3.80 (d, J = 11.2 Hz, 1H), 3.47 | | | |
| | (s, 3H), 3.19-3.10 (m, 1H), 3.08-2.52 (m, | | | |
| | 12H), 2.48-2.30 (m, 4H), 2.30-2.17 (m, | | | |
| | 1H), 2.16-2.05 (m, 1H), 2.01-1.87 (m, | | | |
| | 3H), 1.79-1.69 (m, 2H), 1.68-1.59 (m, | | | |
| | 2H), 1.57-1.47 (m, 2H), 1.45-1.33 (m, 2H). | | | |
| A104 | 1H NMR (400 MHz, DMSO) δ 10.83 (s, 1H), | 829.4 | 829.5 | |
| | 9.15 (s, 1H), 8.49 (d, J = 8.2 Hz, 1H), 7.56 (d, | | | |
| | J = 8.1 Hz, 1H), 7.30 (d, J = 8.4 Hz, 1H), 7.16- | | | |
| | 7.00 (m, 3H), 6.75 (d, J = 6.0 Hz, 2H), 6.65- | | | |
| | 6.42 (m, 3H), 6.26-6.10 (m, 2H), 4.78- | | | |
| | 4.61 (m, 2H), 4.43 (d, J = 8.4 Hz, 1H), 4.13- | | | |
| | 4.03 (m, 1H), 3.76 (d, J = 11.6 Hz, 1H), 3.30- | | | |
| | 3.11 (m, 4H), 3.04-2.93 (m, 5H), 2.92- | | | |
| | 2.66 (m, 10H), 2.45-2.33 (m, 2H), 2.21- | | | |
| | 2.06 (m, 3H), 2.01-1.89 (m, 3H), 1.79- | | | |
| | 1.62 (m, 4H), 1.59-1.53 (m, 2H), 1.48- | | | |
| | 1.37 (m, 2H). | | | |
| A105 | ¹H NMR (400 MHz, DMSO) δ 10.79 (s, 1H), | 771.4 | 771.4 | |
| | 9.10 (d, J = 16.1 Hz, 2H), 7.07 (d, J = 6.8 Hz, | | | |
| | 3H), 6.93 (t, J = 8.8 Hz, 1H), 6.72 (d, J = 2.9 | | | |
| | Hz, 2H), 6.62-6.51 (m, 2H), 6.50-6.43 (m, | | | |
| | 3H), 6.39 (s, 2H), 6.14 (d, J = 16.4 Hz, 1H), | | | |
| | 4.66 (d, J = 5.1 Hz, 1H), 4.31 (s, 1H), 3.21 | | | |
| | (dd, J = 22.7, 2.3 Hz, 4H), 3.05 (d, J = 4.8 Hz, | | | |
| | 4H), 2.96 (s, 3H), 2.91 (d, J = 11.8 Hz, 2H), | | | |
| | 2.82-2.66 (m, 3H), 2.54 (s, 3H), 2.35-2.31 | | | |
| | (m, 1H), 2.18-1.99 (m, 4H), 1.93-1.81 (m, | | | |
| | 5H), 1.71 (s, 2H), 1.65-1.53 (m, 5H) | | | |
| A106 | ¹H NMR (400 MHz, DMSO) δ 10.80 (s, 1H), | 771.4 | 771.4 | |
| | 9.07 (s, 2H), 7.07 (d, J = 6.6 Hz, 3H), 6.93 (t, | | | |
| | J = 8.7 Hz, 1H), 6.72 (d, J = 5.9 Hz, 2H), 6.53 | | | |
| | (dd, J = 19.7, 11.2 Hz, 3H), 6.49-6.42 (m, | | | |
| | 3H), 6.39 (s, 1H), 6.22-6.04 (m, 1H), 4.65 | | | |
| | (d, J = 5.2 Hz, 1H), 4.31 (dd, J = 9.1, 4.6 Hz, | | | |
| | 1H), 3.25-3.15 (m, 4H), 3.05 (dd, J = 12.3, | | | |
| | 6.2 Hz, 4H), 2.95 (s, 3H), 2.91-2.83 (m, | | | |
| | 2H), 2.80-2.67 (m, 3H), 2.63-2.53 (m, | | | |
| | 3H), 2.33 (s, 1H), 2.04 (dd, J = 11.7, 7.7 Hz, | | | |
| | 4H), 1.94-1.77 (m, 5H), 1.75-1.67 (m, | | | |
| | 2H), 1.63-1.51 (m, 5H) | | | |
| A107 | 1H NMR (400 M Hz, DMSO-d6) δ 10.83 (s, | 811.4 | 811.4 | |
| | 1H), 9.88 (brs, 1H), 9.06 (s, 1H), 8.54 (d, J = | | | |
| | 8.8 Hz, 1H), 7.63 (d, J = 8.0 Hz, 1H), 7.49- | | | |
| | 7.46 (m, 1H), 7.09-7.05 (m, 3H), 6.73-6.71 | | | |
| | (m, 2H), 6.57-6.55 (m, 2H), 6.52 (s, 1H), | | | |
| | 6.39-6.32 (m, 2H), 6.13-6.12 (m, 1H), 4.76- | | | |
| | 4.69 (m, 1H), 4.65 (d, J = 4.0 Hz, 1H), 4.50 | | | |
| | (d, J = 10.4 Hz, 1H), 4.22-4.14 (m, 2H), 3.33- | | | |
| | 3.20 (m, 6H), 3.13-3.10 (m, 2H), 3.06-3.02 | | | |
| | (m, 4H), 2.94 (s, 3H), 2.79-2.73 (m, 2H), | | | |
| | 2.69-2.67 (m, 1H), 2.22-2.14 (m, 3H), 2.05- | | | |
| | 1.96 (m, 3H), 1.71-1.67 (m, 2H), 1.59-1.54 | | | |
| | (m, 6H) | | | |
| A108 | 1H NMR (400 MHz, MeOD) δ 10.84 (s, | 811.4 | 811.5 | |
| | 1H), 9.94 (brs, 1H), 9.06 (brs, 1H), 8.55 (d, J = | | | |
| | 8.4 Hz, 1H), 7.63 (d, J = 7.8 Hz, 1H), 7.47 | | | |
| | (d, J = 6.4 Hz, 1H), 7.11-7.05 (m, 4H), 6.72 | | | |
| | (d, J = 7.8 Hz, 2H), 6.58-6.53 (m, 2H), 6.52 (s, | | | |
| | 1H), 6.46-6.43 (m, 1H), 6.17-6.15 (m, 1H), | | | |
| | 4.76-4.73 (m, 1H), 4.68-4.65 (m, 1H), 4.53- | | | |
| | 4.50 (m, 1H), 4.20-4.16 (m, 2H), 3.24-3.20 | | | |
| | (m, 4H), 3.11-3.06 (m, 2H), 2.95 (s, 3H), | | | |
| | 2.77-2.67 (m, 1H), 2.20-2.16 (m, 2H), 2.08- | | | |
| | 1.98 (m, 4H), 1.73-1.68 (m, 2H), 1.60-1.55 | | | |
| | (m, 6H), 1.29-1.21 (m, 8H), 0.87-0.82 (m, 1H) | | | |
| A109 | ¹H NMR (400 MHz, DMSO) δ 10.81 (s, 1H), | 771.4 | 771.5 | |
| | 9.07 (s, 2H), 7.20-7.00 (m, 4H), 6.97-6.88 | | | |
| | (m, 1H), 6.80 (d, J = 10.8 Hz, 2H), 6.73 (d, J = | | | |
| | 7.9 Hz, 2H), 6.60-6.50 (m, 2H), 6.45 (d, J = | | | |
| | 8.6 Hz, 1H), 6.39 (s, 1H), 6.16 (s, 1H), 5.51 | | | |
| | (s, 1H), 4.65 (d, J = 5.2 Hz, 1H), 4.37 (s, 1H), | | | |
| | 3.19 (s, 4H), 3.08 (dd, J = 17.8, 11.0 Hz, 3H), | | | |
| | 2.94-2.85 (m, 3H), 2.67 (s, 3H), 2.59 (s, | | | |
| | 1H), 2.33 (s, 1H), 2.22-2.09 (m, 1H), 2.05 | | | |
| | (dd, J = 14.6, 7.5 Hz, 4H), 2.00-1.85 (m, | | | |
| | 3H), 1.83-1.69 (m, 4H), 1.62-1.53 (m, | | | |
| | 5H), 1.24 (s, 2H), 1.18 (t, J = 7.3 Hz, 1H) | | | |
| A110 | 1H NMR (400 M Hz, DMSO-d6) δ 7.08-7.02 | 753.4 | 753.4 | |
| | (m, 5H), 6.79-6.74 (m, 4H), 6.72-6.70 (m, | | | |
| | 2H), 6.63 (s, 1H), 6.59-6.57 (m, 1H), 6.49- | | | |
| | 6.47 (m, 2H), 4.28-4.24 (m, 1H), 3.58-3.55 | | | |
| | (m, 2H), 3.48-3.46 (m, 1H), 3.26-3.24 (m, | | | |
| | 4H), 3.13-3.12 (m, 2H), 3.08-3.06 (m, 3H), | | | |
| | 3.05-2.99 (m, 3H), 2.81-2.73 (m, 4H), 2.32- | | | |
| | 2.22 (m, 4H), 2.07-2.00 (m, 2H), 1.95-1.87 | | | |
| | (m, 4H), 1.85-1.76 (m, 4H), 1.72-1.66 (m, 4H) | | | |
| A111 | 1H NMR (400 MHz, DMSO) δ 10.82 (s, 1H), | 829.4 | 829.5 | |
| | 9.11 (s, 1H), 8.48 (d, J = 8.2 Hz, 1H), 7.56 (d, | | | |
| | J = 8.0 Hz, 1H), 7.29 (d, J = 8.2 Hz, 1H), 7.08 | | | |
| | (M, 3H), 6.75 (d, J = 6.8 Hz, 2H), 6.62-6.51 | | | |
| | (m, 2H), 6.51-6.45 (m, 1H), 6.26-6.11 (m, | | | |
| | 2H), 4.77-4.63 (m, 2H), 4.43 (d, J = 10.8 | | | |
| | Hz, 1H), 4.12-4.01 (m, 1H), 3.76 (d, J = | | | |
| | 11.6 Hz, 1H), 3.27-3.12 (m, 3H), 3.01- | | | |
| | 2.92 (m, 5H), 2.92-2.69 (m, 9H), 2.46- | | | |
| | 2.36 (m, 2H), 2.22-2.06 (m, 3H), 2.01- | | | |
| | 1.90 (m, 3H), 1.75-1.53 (m, 6H), 1.47- | | | |
| | 1.38 (m, 2H). | | | |
| A112 | ¹H NMR (400 MHz, DMSO) δ 10.95 (s, 1H), | 793.4 | 793.4 | |
| | 9.16 (s, 1H), 7.24 (d, J = 8.2 Hz, 1H), 7.18 | | | |
| | (dd, J = 13.0, 5.7 Hz, 3H), 7.11 (s, 1H), 7.02 | | | |
| | (s, 1H), 6.90 (d, J = 7.2 Hz, 2H), 6.66 (d, J = | | | |
| | 8.4 Hz, 1H), 6.61 (d, J = 1.8 Hz, 1H), 6.58- | | | |
| | 6.45 (m, 3H), 5.04 (dd, J = 13.0, 5.0 Hz, 1H), | | | |
| | 4.41 (d, J = 10.0 Hz, 1H), 4.28 (d, J = 17.0 | | | |
| | Hz, 1H), 4.14 (dd, J = 11.0, 6.1 Hz, 2H), 4.06 | | | |
| | (dd, J = 10.0, 5.6 Hz, 1H), 3.52 (s, 3H), 3.41 | | | |
| | (d, J = 9.9 Hz, 2H), 3.29 (s, 1H), 3.23 (s, 3H), | | | |
| | 3.13-3.00 (m, 2H), 2.95 (dd, J = 12.8, 6.0 | | | |
| | Hz, 2H), 2.88 (dd, J = 17.6, 5.4 Hz, 2H), 2.67 | | | |
| | (s, 1H), 2.60 (s, 1H), 2.56 (s, 1H), 2.49-2.28 | | | |
| | (m, 2H), 2.02 (dd, J = 11.8, 5.5 Hz, 3H), 1.96- | | | |
| | 1.89 (m, 1H), 1.76 (dd, J = 13.6, 6.1 Hz, | | | |
| | 1H), 1.56 (s, 4H), 1.44 (s, 2H) | | | |
| A113 | ¹H NMR (400 MHz, DMSO) δ 10.95 (s, 1H), | 793.4 | 793.4 | |
| | 9.20 (s, 1H), 7.23 (dd, J = 17.6, 6.2 Hz, 3H), | | | |
| | 7.17 (d, J = 15.6 Hz, 3H), 7.02 (d, J = 51.1 | | | |
| | Hz, 2H), 6.93 (d, J = 7.0 Hz, 2H), 6.67 (d, J = | | | |
| | 8.6 Hz, 1H), 6.62 (s, 1H), 6.51 (d, J = 9.1 Hz, | | | |
| | 1H), 5.04 (dd, J = 13.1, 5.1 Hz, 1H), 4.42 (d, | | | |
| | J = 10.3 Hz, 1H), 4.30 (s, 1H), 4.25 (s, 1H), | | | |
| | 4.18 (d, J = 15.1 Hz, 3H), 4.13-4.02 (m, | | | |
| | 2H), 3.27 (s, 4H), 3.17 (d, J = 13.8 Hz, 1H), | | | |
| | 3.08 (d, J = 10.0 Hz, 2H), 3.02 (d, J = 3.9 Hz, | | | |
| | 1H), 3.00-2.75 (m, 5H), 2.72 (dd, J = 12.0, | | | |
| | 3.4 Hz, 1H), 2.67 (s, 1H), 2.60 (s, 1H), 2.41 | | | |
| | (dd, J = 12.7, 2.5 Hz, 1H), 1.99 (dd, J = 28.3, | | | |
| | 8.8 Hz, 5H), 1.78 (d, J = 13.5 Hz, 1H), 1.59 | | | |
| | (d, J = 6.5 Hz, 4H), 1.46 (s, 2H), 1.19 (dd, J = | | | |
| | 19.1, 11.5 Hz, 1H) | | | |
| A114 | 1H NMR (400 MHz, MeOD) δ 10.83 (s, | 782.4 | 782.5 | |
| | 1H), 9.15 (s, 1H), 8.55 (d, J = 12.0 Hz, 1H), | | | |
| | 7.62 (d, J = 8.0 Hz, 1H), 7.43 (brs, 1H), 7.20- | | | |
| | 7.13 (m, 3H), 7.02 (s, 1H), 6.89 (d, J = 6.8 Hz, | | | |
| | 2H), 6.65 (d, J = 7.6 Hz, 1H), 6.61 (s, 1H), | | | |
| | 6.52-6.48 (m, 3H), 4.75-4.68 (m, 1H), 4.50- | | | |
| | 4.46 (m, 1H), 4.19-4.12 (m, 2H), 3.60-3.49 | | | |
| | (m, 2H), 3.24-3.22 (m, 4H), 3.12-3.07 (m, | | | |
| | 1H), 3.02-3.00 (m, 1H), 2.97-2.96 (m, 2H), | | | |
| | 2.94-2.91 (m, 1H), 2.88-2.82 (m, 2H), 2.68- | | | |
| | 2.65 (m, 1H), 2.35-2.30 (m, 1H), 2.21-2.15 | | | |
| | (m, 1H), 2.05-1.96 (m, 5H), 1.79-1.73 (m, | | | |
| | 1H), 1.59-1.53 (m, 4H), 1.46-1.42 (m, 3H), | | | |
| | 1.26-1.20 (m, 2H) | | | |
| A115 | 1H NMR (400 M Hz, MeOD) δ 10.83 (s, | 782.4 | 782.5 | |
| | 1H), 9.15 (s, 1H), 8.56-8.51 (m, 1H), 7.64- | | | |
| | 7.60 (m, 1H), 7.43 (brs, 1H), 7.20-7.13 (m, | | | |
| | 3H), 7.02 (s, 1H), 6.89 (d, J = 6.8 Hz, 2H), | | | |
| | 6.65 (d, J = 7.6 Hz, 1H), 6.61 (s, 1H), 6.52- | | | |
| | 6.48 (m, 3H), 4.75-4.68 (m, 1H), 4.50-4.46 | | | |
| | (m, 1H), 4.19-4.12 (m, 2H), 3.60-3.49 (m, | | | |
| | 2H), 3.24-3.22 (m, 4H), 3.12-3.07 (m, 1H), | | | |
| | 3.02-3.00 (m, 1H), 2.97-2.96 (m, 2H), 2.94- | | | |
| | 2.91 (m, 1H), 2.88-2.82 (m, 2H), 2.68-2.65 | | | |
| | (m, 1H), 2.35-2.30 (m, 1H), 2.21-2.15 (m, | | | |
| | 1H), 2.05-1.96 (m, 5H), 1.79-1.73 (m, 1H), | | | |
| | 1.59-1.53 (m, 4H), 1.46-1.42 (m, 3H), 1.26- | | | |
| | 1.20 (m, 2H) | | | |
| A116 | ¹H NMR (400 MHz, DMSO) δ 10.98 (s, 1H), | 792.4 | 792.5 | |
| | 9.14 (s, 1H), 7.30 (d, J = 21.4 Hz, 2H), 7.13 | | | |
| | (dd, J = 32.7, 21.2 Hz, 4H), 6.89 (d, J = 6.3 | | | |
| | Hz, 2H), 6.72-6.58 (m, 2H), 6.50 (d, J = 9.6 | | | |
| | Hz, 3H), 5.09 (d, J = 7.7 Hz, 1H), 4.64 (s, | | | |
| | 2H), 4.39 (d, J = 17.4 Hz, 1H), 4.23 (d, J = | | | |
| | 17.5 Hz, 1H), 4.13 (s, 1H), 3.45 (dd, J = 11.0, | | | |
| | 4.3 Hz, 4H), 3.24 (s, 4H), 3.12-2.98 (m, | | | |
| | 2H), 2.98-2.80 (m, 3H), 2.71 (dd, J = 18.6, | | | |
| | 11.0 Hz, 2H), 2.60 (dd, J = 15.5, 4.0 Hz, 2H), | | | |
| | 2.46-2.30 (m, 2H), 2.14-1.92 (m, 7H), | | | |
| | 1.82-1.70 (m, 1H), 1.56 (s, 3H), 1.44 (dd, J = | | | |
| | 6.0, 2.0 Hz, 2H) | | | |
| A117 | ¹H NMR (400 MHz, DMSO) δ 10.98 (s, 1H), | 792.4 | 792.5 | |
| | 9.14 (s, 1H), 8.14 (s, 1H), 7.33 (s, 2H), 7.25- | | | |
| | 7.11 (m, 3H), 7.05 (s, 1H), 6.89 (d, J = 6.9 | | | |
| | Hz, 2H), 6.66 (d, J = 8.5 Hz, 1H), 6.60 (s, | | | |
| | 1H), 6.49 (dt, J = 18.3, 9.0 Hz, 3H), 5.09 (dd, | | | |
| | J = 13.2, 5.3 Hz, 1H), 4.60 (s, 2H), 4.39 (d, J = | | | |
| | 17.1 Hz, 1H), 4.22 (d, J = 17.1 Hz, 1H), | | | |
| | 4.12 (d, J = 4.8 Hz, 1H), 3.23 (s, 6H), 2.94 | | | |
| | (ddd, J = 28.2, 16.6, 4.1 Hz, 6H), 2.62 (t, J = | | | |
| | 19.9 Hz, 3H), 2.42 (dd, J = 13.9, 4.2 Hz, 2H), | | | |
| | 2.13-1.94 (m, 6H), 1.91-1.80 (m, 2H), | | | |
| | 1.80-1.69 (m, 1H), 1.53 (d, J = 19.3 Hz, | | | |
| | 4H), 1.44 (s, 2H) | | | |
| A118 | 1H NMR (400 MHz, DMSO) δ 10.81 (s, 1H), | 749.4 | 749.4 | |
| | 8.31 (s, 1H), 7.09 (d, J = 6.8 Hz, 3H), 6.95- | | | |
| | 6.89 (m, 1H), 6.87-6.80 (m, 1H), 6.75 (t, J = | | | |
| | 8.8 Hz, 3H), 6.60-6.53 (m, 2H), 6.50-6.45 | | | |
| | (m, 1H), 6.24-6.15 (m, 2H), 5.38 (d, J = 6.7 | | | |
| | Hz, 1H), 4.67 (d, J = 4.8 Hz, 1H), 4.41-4.32 | | | |
| | (m, 1H), 3.30-3.22 (m, 4H), 2.98-2.90 (m, | | | |
| | 6H), 2.80-2.72 (m, 1H), 2.64-2.56 (m, | | | |
| | 2H), 2.39-2.33 (m, 1H), 2.21-2.14 (m, | | | |
| | 3H), 2.09-1.92 (m, 4H), 1.82-1.68 (m, | | | |
| | 4H), 1.65-1.55 (m, 4H), 1.30-1.19 (m, 2H). | | | |
| A119 | 1H NMR (400 MHz, DMSO) δ 10.78 (s, 1H), | 749.4 | 749.4 | |
| | 8.23 (s, 1H), 7.12-7.06 (m, 3H), 6.99 (t, J = | | | |
| | 8.8 Hz, 1H), 6.78-6.73 (m, 2H), 6.60-6.53 | | | |
| | (m, 2H), 6.49-6.39 (m, 3H), 6.22-6.15 (m, | | | |
| | 2H), 5.99 (d, J = 7.6 Hz, 1H), 4.67 (d, J = 5.2 | | | |
| | Hz, 1H), 4.34-4.26 (m, 1H), 3.31-3.22 (m, | | | |
| | 4H), 2.97-2.91 (m, 6H), 2.76-2.69 (m, | | | |
| | 1H), 2.64-2.56 (m, 3H), 2.21-2.14 (m, | | | |
| | 3H), 2.12-2.04 (m, 1H), 2.00-1.85 (m, | | | |
| | 3H), 1.81-1.72 (m, 2H), 1.67-1.56 (m, | | | |
| | 7H), 1.29-1.17 (m, 2H). | | | |
| A120 | 1H NMR (400 M Hz, MeOD) δ 10.94 (s, | 793.4 | 793.5 | |
| | 1H), 9.14 (s, 1H), 7.19-7.13 (m, 4H), 7.03 (s, | | | |
| | 2H), 6.89 (d, J = 7.2 Hz, 1H), 6.61 (s, 1H), | | | |
| | 6.53-6.48 (m, 3H), 5.06-5.00 (m, 1H), 4.35- | | | |
| | 4.19 (m, 5H), 4.13-4.12 (m, 1H), 3.56-3.51 | | | |
| | (m, 3H), 3.24-3.21 (m, 3H), 3.11-3.08 (m, | | | |
| | 1H), 2.97-2.88 (m, 4H), 2.87-2.85 (m, 1H), | | | |
| | 2.69-2.67 (m, 1H), 2.38-2.31 (m, 1H), 2.07- | | | |
| | 1.96 (m, 5H), 1.77-1.73 (m, 1H), 1.55 (s, 3H), | | | |
| | 1.43 (s, 2H), 1.29-1.23 (m, 5H) | | | |
| A121 | ¹H NMR (400 MHz, DMSO) δ 10.94 (s, 1H), | 793.4 | 793.5 | |
| | 9.21 (s, 1H), 7.23-7.17 (m, 4H), 7.03 (s, | | | |
| | 1H), 6.93 (d, J = 6.8 Hz, 3H), 6.68 (d, J = 8.4 | | | |
| | Hz, 2H), 6.62 (s, 1H), 6.52 (d, J = 8.1 Hz, | | | |
| | 1H), 5.04 (dd, J = 13.1, 5.1 Hz, 1H), 4.34 (d, | | | |
| | J = 11.7 Hz, 1H), 4.18 (d, J = 16.4 Hz, 3H), | | | |
| | 4.07-3.95 (m, 2H), 3.38 (s, 5H), 3.29 (s, | | | |
| | 3H), 3.09 (s, 2H), 2.98-2.85 (m, 4H), 2.75- | | | |
| | 2.59 (m, 3H), 2.40-2.30 (m, 1H), 2.05- | | | |
| | 1.92 (m, 4H), 1.80 (s, 1H), 1.59 (d, J = 6.9 | | | |
| | Hz, 4H), 1.47 (s, 2H) | | | |
| A122 | 1H NMR (400 MHz, DMSO) δ 10.97 (s, 1H), | 799.4 | 799.4 | |
| | 8.32 (s, 1H), 7.46 (s, 1H), 7.14-7.06 (m, | | | |
| | 3H), 6.99 (d, J = 9.6 Hz, 1H), 6.76 (d, J = 6.2 | | | |
| | Hz, 2H), 6.62-6.53 (m, 2H), 6.50-6.42 (m, | | | |
| | 1H), 6.23-6.15 (m, 2H), 5.12-5.03 (m, | | | |
| | 1H), 4.67 (d, J = 5.2 Hz, 1H), 4.50-4.44 (m, | | | |
| | 2H), 4.36-4.30 (m, 1H), 4.24-4.18 (m, | | | |
| | 1H), 3.29-3.19 (m, 4H), 2.97-2.82 (m, | | | |
| | 8H), 2.62-2.54 (m, 2H), 2.42-2.34 (m, | | | |
| | 1H), 2.23-2.14 (m, 3H), 2.01-1.88 (m, | | | |
| | 5H), 1.81-1.74 (m, 2H), 1.70-1.59 (m, | | | |
| | 4H), 1.30-1.18 (m, 2H). | | | |
| A123 | ¹H NMR (400 MHz, DMSO) δ 10.92 (s, 1H), | 793.4 | 793.4 | |
| | 9.13 (s, 1H), 7.25-7.11 (m, 4H), 7.09-6.85 | | | |
| | (m, 4H), 6.75-6.42 (m, 4H), 6.00 (d, J = 8.6 | | | |
| | Hz, 1H), 5.12-4.91 (m, 1H), 4.47-3.74 (m, | | | |
| | 7H), 2.99-2.86 (m, 5H), 2.80-2.54 (m, | | | |
| | 3H), 2.47-2.36 (m, 1H), 2.20-1.94 (m, | | | |
| | 5H), 1.87-1.62 (m, 6H), 1.57-1.38 (m, | | | |
| | 3H), 0.98-0.84 (m, 2H). | | | |
| A124 | ¹H NMR (400 MHz, DMSO) δ 10.93 (s, 1H), | 793.4 | 793.4 | |
| | 8.35 (s, 1H), 7.26-7.08 (m, 3H), 7.06-6.85 | | | |
| | (m, 5H), 6.68-6.48 (m, 4H), 6.00 (d, J = 8.6 | | | |
| | Hz, 1H), 5.08-4.97 (m, 1H), 4.32-4.15 (m, | | | |
| | 3H), 3.95-3.77 (m, 3H), 3.55-3.45 (m, | | | |
| | 4H), 3.33-3.14 (m, 3H), 2.92 (d, J = 11.4 | | | |
| | Hz, 4H), 2.16-1.94 (m, 6H), 1.85-1.66 (m, | | | |
| | 6H), 1.42 (t, J = 12.4 Hz, 3H), 1.24 (s, 2H), | | | |
| | 0.97-0.86 (m, 2H). | | | |
| A125 | ¹H NMR (400 MHz, MeOD) δ 7.38 (s, 1H), | 792.4 | 792.4 | |
| | 7.14 (dd, J = 14.2, 5.6 Hz, 4H), 7.06 (d, J = | | | |
| | 2.4 Hz, 1H), 6.88 (d, J = 6.8 Hz, 2H), 6.71 (d, | | | |
| | J = 8.4 Hz, 1H), 6.64 (dd, J = 8.3, 2.3 Hz, | | | |
| | 2H), 6.55 (s, 1H), 6.49 (d, J = 8.8 Hz, 1H), | | | |
| | 5.12 (dd, J = 13.3, 5.2 Hz, 1H), 4.56 (s, 2H), | | | |
| | 4.39 (t, J = 12.0 Hz, 2H), 4.17 (d, J = 4.7 Hz, | | | |
| | 1H), 3.35 (dd, J = 11.8, 5.4 Hz, 5H), 3.25 (d, | | | |
| | J = 7.6 Hz, 3H), 3.03 (d, J = 6.8 Hz, 4H), 2.88 | | | |
| | (dd, J = 19.0, 5.9 Hz, 2H), 2.77 (d, J = 13.6 | | | |
| | Hz, 3H), 2.68 (d, J = 11.4 Hz, 1H), 2.47 (dd, J = | | | |
| | 13.5, 4.7 Hz, 1H), 2.14 (t, J = 12.1 Hz, 7H), | | | |
| | 1.97 (d, J = 14.7 Hz, 2H), 1.85 (d, J = 5.3 Hz, | | | |
| | 1H), 1.70 (s, 2H), 1.62 (t, J = 9.0 Hz, 2H), | | | |
| | 1.56 (d, J = 3.8 Hz, 2H) | | | |
| A126 | ¹H NMR (400 MHz, MeOD) δ 7.39 (s, 1H), | 792.4 | 792.4 | |
| | 7.20-7.10 (m, 4H), 7.06 (d, J = 2.2 Hz, 1H), | | | |
| | 6.88 (d, J = 6.9 Hz, 2H), 6.71 (d, J = 8.3 Hz, | | | |
| | 1H), 6.65 (d, J = 6.6 Hz, 2H), 6.58-6.51 (m, | | | |
| | 1H), 6.49 (d, J = 8.6 Hz, 1H), 5.12 (dd, J = | | | |
| | 13.3, 5.1 Hz, 1H), 4.57 (s, 4H), 4.47-4.32 | | | |
| | (m, 2H), 4.17 (d, J = 4.8 Hz, 1H), 3.34 (s, | | | |
| | 6H), 3.28-3.23 (m, 3H), 3.04 (dd, J = 12.8, | | | |
| | 5.6 Hz, 4H), 2.96-2.82 (m, 3H), 2.82-2.63 | | | |
| | (m, 3H), 2.47 (dd, J = 12.7, 3.8 Hz, 1H), 2.31- | | | |
| | 2.05 (m, 7H), 1.98 (d, J = 16.6 Hz, 2H), | | | |
| | 1.82 (d, J = 16.1 Hz, 1H), 1.69 (s, 2H), 1.63 | | | |
| | (t, J = 9.5 Hz, 2H), 1.56 (s, 2H) | | | |
| A127 | 1H NMR (400 MHz, DMSO) δ 10.83 (s, 1H), | 782.4 | 782.4 | |
| | 8.49 (d, J = 8.2 Hz, 1H), 8.31 (s, 1H), 7.56 (d, | | | |
| | J = 8.2 Hz, 1H), 7.29 (d, J = 8.4 Hz, 1H), 7.22- | | | |
| | 7.12 (m, 3H), 7.01 (d, J = 11.8 Hz, 1H), | | | |
| | 6.88 (d, J = 7.2 Hz, 2H), 6.70-6.59 (m, 2H), | | | |
| | 6.49 (d, J = 8.2 Hz, 2H), 6.00 (d, J = 8.4 Hz, | | | |
| | 1H), 4.78-4.67 (m, 1H), 4.44 (d, J = 8.6 Hz, | | | |
| | 1H), 4.15-4.04 (m, 2H), 3.77 (d, J = 10.4 | | | |
| | Hz, 1H), 3.30 (d, J = 10.8 Hz, 3H), 3.19 (s, | | | |
| | 1H), 2.92 (d, J = 10.6 Hz, 3H), 2.75 (d, J = | | | |
| | 8.8 Hz, 2H), 2.19-1.96 (m, 7H), 1.85-1.65 | | | |
| | (m, 7H), 1.42 (t, J = 11.6 Hz, 4H), 1.24 (s, | | | |
| | 1H), 0.99-0.86 (m, 2H). | | | |
| A128 | ¹H NMR (400 MHz, DMSO) δ 10.97 (s, 1H), | 792.4 | 792.4 | |
| | 8.30 (s, 1H), 7.45 (s, 1H), 7.22-7.13 (m, | | | |
| | 3H), 7.00 (s, 2H), 6.88 (d, J = 7.2 Hz, 2H), | | | |
| | 6.71-6.56 (m, 2H), 6.50 (d, J = 8.4 Hz, 2H), | | | |
| | 6.00 (d, J = 8.6 Hz, 1H), 5.13-5.04 (m, 1H), | | | |
| | 4.45 (s, 2H), 4.39-4.10 (m, 4H), 3.31 (d, J = | | | |
| | 13.0 Hz, 5H), 3.00-2.79 (m, 5H), 2.10 (d, J = | | | |
| | 6.6 Hz, 2H), 2.00-1.79 (m, 8H), 1.67 (s, | | | |
| | 4H), 1.49-1.38 (m, 3H), 1.24 (s, 2H), 0.89 | | | |
| | (d, J = 12.2 Hz, 2H). | | | |
| A129 | 1H NMR (400 MHz, DMSO) δ 10.97 (s, 1H), | 839.4 | 839.4 | |
| | 8.30 (s, 1H), 7.44 (s, 1H), 7.12-6.97 (m, | | | |
| | 4H), 6.75 (d, J = 6.4 Hz, 2H), 6.60-6.44 (m, | | | |
| | 3H), 6.22-6.12 (m, 2H), 5.07 (dd, J = 13.2, | | | |
| | 4.8 Hz, 1H), 4.67 (d, J = 4.8 Hz, 1H), 4.45 (s, | | | |
| | 2H), 4.36-4.18 (m, 2H), 3.25 (d, J = 9.2 Hz, | | | |
| | 1H), 2.98-2.75 (m, 12H), 2.64-2.55 (m, | | | |
| | 1H), 2.46-2.29 (m, 4H), 2.19-2.10 (m, | | | |
| | 1H), 2.03-1.85 (m, 7H), 1.70-1.54 (m, | | | |
| | 7H), 1.46-1.38 (m, 2H) | | | |
| A130 | 1H NMR (400 MHz, DMSO) δ 10.93 (s, 1H), | 793.4 | 793.4 | |
| | 8.41 (s, 1H), 7.23-7.13 (m, 3H), 7.07-6.83 | | | |
| | (m, 5H), 6.70-6.47 (m, 4H), 6.00 (d, J = 8.6 | | | |
| | Hz, 1H), 5.07-4.94 (m, 1H), 4.31-4.09 (m, | | | |
| | 4H), 3.92-3.78 (m, 2H), 3.51 (s, 2H), 3.18 | | | |
| | (s, 3H), 2.98-2.87 (m, 5H), 2.77-2.67 (m, | | | |
| | 1H), 2.34 (d, J = 12.0 Hz, 1H), 2.12-1.95 | | | |
| | (m, 6H), 1.74 (d, J = 11.4 Hz, 4H), 1.51- | | | |
| | 1.38 (m, 3H), 1.24 (s, 3H), 0.97-0.87 (m, 2H). | | | |
| A131 | ¹H NMR (400 MHz, DMSO) δ 10.97 (s, 1H), | 792.4 | 792.4 | |
| | 8.31 (s, 2H), 7.39 (d, J = 7.4 Hz, 1H), 7.31- | | | |
| | 7.12 (m, 4H), 6.99 (s, 1H), 6.88 (d, J = 7.4 | | | |
| | Hz, 2H), 6.68-6.59 (m, 2H), 6.50 (d, J = 8.2 | | | |
| | Hz, 2H), 6.00 (d, J = 8.4 Hz, 1H), 5.09-5.00 | | | |
| | (m, 1H), 4.52 (d, J = 9.6 Hz, 2H), 4.37 (d, J = | | | |
| | 11.2 Hz, 1H), 4.25-4.10 (m, 2H), 3.04- | | | |
| | 2.88 (m, 4H), 2.79 (d, J = 7.0 Hz, 3H), 2.12- | | | |
| | 2.00 (m, 3H), 1.98 (d, J = 6.8 Hz, 2H), 1.81- | | | |
| | 1.64 (m, 7H), 1.49-1.36 (m, 4H), 1.24 (s, | | | |
| | 5H), 1.00-0.83 (m, 3H). | | | |
| A132 | 1H NMR (400 MHz, DMSO) δ 8.31 (s, 1H), | 836.4 | 836.4 | |
| | 7.18 (d, J = 8.2 Hz, 1H), 7.09-7.05 (m, 3H), | | | |
| | 7.01 (d, J = 8.4 Hz, 1H), 6.76-6.70 (m, 2H), | | | |
| | 6.57-6.52 (m, 2H), 6.47-6.43 (m, 1H), | | | |
| | 6.36 (d, J = 8.4 Hz, 1H), 6.32-6.27 (m, 1H), | | | |
| | 6.10 (d, J = 1.8 Hz, 1H), 5.14-5.06 (m, 1H), | | | |
| | 4.64 (d, J = 5.2 Hz, 1H), 4.37-4.30 (m, 1H), | | | |
| | 4.29-4.21 (m, 1H), 4.15-4.06 (m, 1H), | | | |
| | 4.00-3.93 (m, 1H), 3.83-3.79 (m, 1H), | | | |
| | 3.22-3.14 (m, 2H), 3.02-2.97 (m, 6H), | | | |
| | 2.94-2.88 (m, 9H), 2.77-2.70 (m, 2H), | | | |
| | 2.45-2.37 (m, 3H), 2.18-2.08 (m, 2H), | | | |
| | 1.97-1.91 (m, 3H), 1.79-1.70 (m, 1H), | | | |
| | 1.66-1.57 (m, 3H), 1.54-1.48 (m, 2H), | | | |
| | 1.46-1.38 (m, 2H). | | | |
| A133 | ¹H NMR (400 MHz, DMSO-d6) δ 10.98 (s, 1 | 838.0 | 838.0 | |
| | H), 9.10 (s, 1 H), 7.01-7.30 (m, 5 H), 6.77 | | | |
| | (br d, J = 6.63 Hz, 2 H), 6.57-6.65 (m, 2 H), | | | |
| | 6.50 (br d, J = 7.63 Hz, 1 H), 6.31-6.45 (m, 2 | | | |
| | H), 6.16 (s, 1 H), 5.08 (br dd, J = 13.45, 4.82 | | | |
| | Hz, 1 H), 4.69 (br d, J = 4.75 Hz, 1 H), 4.41 | | | |
| | (br d, J = 9.01 Hz, 1 H), 4.31 (br d, J = 16.76 | | | |
| | Hz, 1 H), 4.15 (br d, J = 16.88 Hz, 1 H), 3.81- | | | |
| | 4.07 (m, 3 H), 3.46-3.53 (m, 1 H), 3.09- | | | |
| | 3.29 (m, 6 H), 2.87-3.08 (m, 8 H), 2.78 (br | | | |
| | d, J = 9.13 Hz, 1 H), 2.58-2.67 (m, 2 H), | | | |
| | 2.34-2.49 (m, 3 H), 2.14-2.28 (m, 2 H), | | | |
| | 1.99 (br dd, J = 11.44, 7.44 Hz, 2 H), 1.56- | | | |
| | 1.84 (m, 6 H), 1.40 (br dd, J = 12.26, 8.38 Hz, 1 H). | | | |
| A134 | | 838.0 | 838.0 | |
| A135 | ¹H NMR (400 MHz, DMSO-d6) 10.94 (s, 1H), | 838.4 | 838.4 | |
| | 9.06 (s, 1H), 7.11-7.02 (m, 4H), 6.94 (s, 1H), | | | |
| | 6.73 (br d, J = 6.3 Hz, 2H), 6.60-6.53 (m, | | | |
| | 2H), 6.46 (br d, J = 8.3 Hz, 1H), 6.40-6.28 | | | |
| | (m, 2H), 6.12 (s, 1H), 5.03 (br dd, J = 4.8, 13.2 | | | |
| | Hz, 1H), 4.65 (br d, J = 4.9 Hz, 1H), 4.33- | | | |
| | 4.21 (m, 2H), 4.19-4.10 (m, 1H), 3.95-3.78 | | | |
| | (m, 3H), 3.45 (br t, J = 7.9 Hz, 1H), 3.24-3.16 | | | |
| | (m, 2H), 3.12-3.00 (m, 5H), 2.99-2.88 (m, | | | |
| | 7H), 2.82-2.74 (m, 1H), 2.58 (br d, J = 14.8 | | | |
| | Hz, 2H), 2.42-2.28 (m, 3H), 2.18 (br dd, | | | |
| | J = 6.5, 11.3 Hz, 1H), 2.12-2.03 (m, 1H), 2.01- | | | |
| | 1.90 (m, 2H), 1.75 (br t, J = 10.9 Hz, 1H), | | | |
| | 1.63 (br dd, J = 5.0, 13.4 Hz, 5H), 1.35 (br dd, | | | |
| | J = 8.0, 12.5 Hz, 1H). | | | |
| A136 | ¹H NMR (400 MHz, DMSO-d₆) δ 10.93 (s, 1 | 838.0 | 838.0 | |
| | H), 9.06 (s, 1 H), 6.88-7.14 (m, 5 H), 6.72 | | | |
| | (br d, J = 6.25 Hz, 2 H), 6.51-6.62 (m, 2 H), | | | |
| | 6.45 (dd, J = 8.25, 1.88 Hz, 1 H), 6.24-6.39 | | | |
| | (m, 2 H), 6.11 (s, 1 H), 5.02 (br dd, J = 13.13, | | | |
| | 5.00 Hz, 1 H), 4.64 (br d, J = 4.88 Hz, 1 H), | | | |
| | 4.07-4.33 (m, 3 H), 3.73-3.96 (m, 3 H), | | | |
| | 3.43-3.47 (m, 1 H), 3.04-3.25 (m, 7 H), | | | |
| | 2.87-3.01 (m, 7 H), 2.76 (br t, J = 10.69 Hz, | | | |
| | 1 H), 2.53-2.63 (m, 2 H), 2.26-2.42 (m, 3 | | | |
| | H), 2.10-2.25 (m, 2 H), 1.86-2.00 (m, 2 H), | | | |
| | 1.53-1.72 (m, 6 H), 1.34 (br dd, J = 12.63, | | | |
| | 8.38 Hz, 1 H). | | | |
| A137 | 1H NMR (400 MHz, DMSO) δ 10.94 (s, 1H), | 809.4 | 809.4 | |
| | 8.43 (s, 1H), 7.22-7.09 (m, 4H), 7.07-6.98 | | | |
| | (m, 2H), 6.88 (d, J = 6.6 Hz, 2H), 6.67 (d, J = | | | |
| | 8.6 Hz, 1H), 6.60 (s, 1H), 6.54-6.44 (m, | | | |
| | 3H), 5.07-4.98 (m, 1H), 4.39-4.20 (m, | | | |
| | 2H), 4.16-4.04 (m, 2H), 4.00-3.77 (m, | | | |
| | 3H), 3.50-3.41 (m, 4H), 3.21-3.05 (m, | | | |
| | 2H), 3.03-2.83 (m, 5H), 2.79-2.54 (m, | | | |
| | 2H), 2.41-2.31 (m, 4H), 2.16-1.86 (m, | | | |
| | 5H), 1.82-1.60 (m, 3H), 1.56-1.42 (m, | | | |
| | 4H), 1.35-1.20 (m, 2H). | | | |
| A138 | 1H NMR (400 MHz, DMSO) δ 10.94 (s, 1H), | 809.4 | 809.4 | |
| | 8.31 (s, 1H), 7.22-7.10 (m, 4H), 7.05-6.97 | | | |
| | (m, 2H), 6.88 (d, J = 7.2 Hz, 2H), 6.69-6.56 | | | |
| | (m, 2H), 6.54-6.44 (m, 3H), 5.07-4.98 (m, | | | |
| | 1H), 4.35 (d, J = 9.8 Hz, 1H), 4.30-4.20 (m, | | | |
| | 1H), 4.13-4.05 (m, 2H), 4.00-3.77 (m, | | | |
| | 3H), 3.53-3.39 (m, 4H), 3.22-3.06 (m, | | | |
| | 2H), 3.04-2.84 (m, 5H), 2.77-2.66 (m, | | | |
| | 1H), 2.63-2.52 (m, 2H), 2.45-2.25 (m, | | | |
| | 4H), 2.15-1.90 (m, 4H), 1.81-1.62 (m, | | | |
| | 2H), 1.59-1.39 (m, 4H), 1.38-1.24 (m, 1H). | | | |
| A139 | 1H NMR (400 MHz, DMSO) δ 10.94 (s, 1H), | 794.4 | 794.5 | |
| | 8.31 (s, 1H), 7.25-7.16 (m, 6H), 6.97 (dd, J = | | | |
| | 19.6, 7.6 Hz, 3H), 6.70 (d, J = 8.4 Hz, 1H), | | | |
| | 6.61 (s, 1H), 6.52 (dd, J = 8.4, 2.4 Hz, 1H), | | | |
| | 5.03 (dd, J = 13.2, 5.2 Hz, 1H), 4.30 (dd, J = | | | |
| | 33.6, 12.8 Hz, 2H), 4.13-4.07 (m, 2H), 3.98- | | | |
| | 3.82 (m, 2H), 3.50 (d, J = 36.4 Hz, 6H), | | | |
| | 2.94-2.84 (m, 4H), 2.68 (dd, J = 34.4, 23.4 | | | |
| | Hz, 2H), 2.42 (dd, J = 26.7, 9.0 Hz, 4H), 2.13- | | | |
| | 1.69 (m, 8H), 1.43 (dd, J = 30.5, 20.8 Hz, 6H). | | | |
| A140 | 1H NMR (400 MHz, DMSO) δ 10.94 (s, 1H), | 794.4 | 794.6 | |
| | 8.38 (s, 1H), 7.25-7.15 (m, 6H), 6.98 (dd, J = | | | |
| | 19.6, 7.6 Hz, 3H), 6.70 (d, J = 8.4 Hz, 1H), | | | |
| | 6.60 (s, 1H), 6.52 (d, J = 8.0 Hz, 1H), 5.03 | | | |
| | (dd, J = 13.2, 4.8 Hz, 1H), 4.30 (dd, J = 34.4, | | | |
| | 13.2 Hz, 2H), 4.09 (d, J = 16.8 Hz, 2H), 3.95 | | | |
| | (t, J = 9.6 Hz, 1H), 3.80 (d, J = 12.4 Hz, 1H), | | | |
| | 3.55-3.43 (m, 6H), 3.24-3.08 (m, 2H), | | | |
| | 2.96-2.85 (m, 4H), 2.75-2.56 (m, 2H), | | | |
| | 2.42-2.37 (m, 2H), 2.16-1.86 (m, 6H), | | | |
| | 1.75 (dd, J = 26.2, 15.1 Hz, 2H), 1.43 (dd, J = | | | |
| | 31.0, 21.4 Hz, 6H). | | | |
| A141 | 1H NMR (400 MHz, DMSO) δ 10.94 (s, 1H), | 793.4 | 793.6 | |
| | 8.38 (s, 1H), 7.25-7.15 (m, 6H), 6.98 (dd, J = | | | |
| | 19.6, 7.6 Hz, 3H), 6.70 (d, J = 8.4 Hz, 1H), | | | |
| | 6.60 (s, 1H), 6.52 (d, J = 8.0 Hz, 1H), 5.03 | | | |
| | (dd, J = 13.2, 5.2 Hz, 1H), 4.30 (dd, J = 34.0, | | | |
| | 13.2 Hz, 2H), 4.09 (d, J = 16.8 Hz, 2H), 3.95 | | | |
| | (t, J = 9.6 Hz, 1H), 3.83-3.77 (m, 1H), 3.56- | | | |
| | 3.47 (m, 4H), 3.37-3.31 (m, 2H), 3.15 (s, | | | |
| | 2H), 2.94-2.83 (m, 4H), 2.75-2.56 (m, | | | |
| | 2H), 2.45-2.36 (m, 3H), 2.18-1.85 (m, | | | |
| | 6H), 1.82-1.69 (m, 2H), 1.51-1.34 (m, 6H).. | | | |
| A142 | 1H NMR (400 MHz, DMSO) δ 10.93 (s, 1H), | 793.4 | 793.6 | |
| | 9.18 (s, 1H), 7.26-7.16 (m, 5H), 7.03 (s, | | | |
| | 1H), 6.98-6.91 (m, 3H), 6.70 (d, J = 8.4 Hz, | | | |
| | 1H), 6.61 (d, J = 2.4 Hz, 1H), 6.52 (dd, J = | | | |
| | 8.4, 2.4 Hz, 1H), 5.02 (dd, J = 13.2, 4.8 Hz, | | | |
| | 1H), 4.29-4.09 (m, 4H), 3.92-3.86 (m, | | | |
| | 1H), 3.78 (d, J = 11.2 Hz, 1H), 3.58-3.43 | | | |
| | (m, 6H), 3.24-3.05 (m, 2H), 2.99-2.84 (m, | | | |
| | 5H), 2.78-2.69 (m, 1H), 2.44-2.29 (m, | | | |
| | 3H), 2.13-1.91 (m, 5H), 1.82-1.66 (m, | | | |
| | 2H), 1.51-1.34 (m, 6H). | | | |
| A143 | 1H NMR (400 MHz, DMSO) δ 10.93 (s, 1H), | 794.4 | 794.7 | |
| | 9.18 (s, 1H), 7.26-7.16 (m, 5H), 7.03 (s, | | | |
| | 1H), 6.98-6.91 (m, 3H), 6.70 (d, J = 8.4 Hz, | | | |
| | 1H), 6.61 (d, J = 2.4 Hz, 1H), 6.52 (dd, J = | | | |
| | 8.4, 2.4 Hz, 1H), 5.02 (dd, J = 13.2, 4.8 Hz, | | | |
| | 1H), 4.29-4.09 (m, 4H), 3.92-3.86 (m, | | | |
| | 1H), 3.78 (d, J = 11.2 Hz, 1H), 3.58-3.43 | | | |
| | (m, 6H), 3.24-3.05 (m, 2H), 2.99-2.84 (m, | | | |
| | 5H), 2.78-2.69 (m, 1H), 2.44-2.29 (m, | | | |
| | 3H), 2.13-1.91 (m, 5H), 1.82-1.66 (m, | | | |
| | 2H), 1.51-1.34 (m, 6H). | | | |
| A1448 | 1H NMR (400 MHz, DMSO) δ 10.94 (s, 1H), | 794.4 | 794.5 | |
| | 7.26-7.16 (m, 5H), 7.03 (s, 1H), 6.98-6.92 | | | |
| | (m, 3H), 6.70 (d, J = 8.4 Hz, 1H), 6.61 (s, | | | |
| | 1H), 6.52 (d, J = 8.0 Hz, 1H), 5.02 (dd, J = | | | |
| | 13.2, 4.8 Hz, 1H), 4.38-4.04 (m, 5H), 3.94- | | | |
| | 3.74 (m, 3H), 3.14 (s, 2H), 3.04-2.82 (m, | | | |
| | 6H), 2.78-2.54 (m, 3H), 2.47-2.29 (m, | | | |
| | 4H), 2.14-1.68 (m, 8H), 1.53-1.35 (m, 6H). | | | |
| A145 | 1H NMR (400 MHz, DMSO) δ 10.94 (s, 1H), | 810.4 | 810.4 | |
| | 8.26 (s, 1H), 7.20-6.98 (m, 5H), 6.86-6.78 | | | |
| | (m, 2H), 6.61 (d, J = 8.8 Hz, 2H), 6.55-6.42 | | | |
| | (m, 3H), 6.25 (d, J = 13.6 Hz, 1H), 5.02 (dd, J = | | | |
| | 13.2, 5.2 Hz, 1H), 4.47-4.22 (m, 3H), 4.10 | | | |
| | (d, J = 16.8 Hz, 1H), 3.98-3.91 (m, 1H), | | | |
| | 3.80 (d, J = 11.2 Hz, 1H), 3.22-3.10 (m, | | | |
| | 2H), 3.03-2.84 (m, 9H), 2.76-2.58 (m, | | | |
| | 2H), 2.49-2.33 (m, 4H), 2.22-2.06 (m, | | | |
| | 2H), 1.99-1.86 (m, 3H), 1.76-1.57 (m, | | | |
| | 4H), 1.52-1.37 (m, 4H). | | | |
| A146 | 1H NMR (400 MHz, DMSO) δ 10.98 (s, 1H), | 809.4 | 809.8 | |
| | 8.31 (s, 1H), 7.32 (dd, J = 47.6, 7.6 Hz, 2H), | | | |
| | 7.13-7.07 (m, 3H), 6.85-6.79 (m, 2H), | | | |
| | 6.63-6.45 (m, 5H), 6.25 (d, J = 12.8 Hz, | | | |
| | 1H), 5.08 (dd, J = 13.2, 4.8 Hz, 1H), 4.57- | | | |
| | 4.33 (m, 4H), 4.21 (d, J = 17.2 Hz, 1H), 3.05- | | | |
| | 2.85 (m, 8H), 2.65-2.53 (m, 2H), 2.47- | | | |
| | 2.33 (m, 4H), 2.10-1.81 (m, 8H), 1.74- | | | |
| | 1.36 (m, 10H). | | | |
| A147 | ¹H NMR (400 MHz, DMSO) δ 10.93 (s, 1H), | 810.4 | 810.4 | |
| | 9.14 (s, 1H), 7.17 (d, J = 8.2 Hz, 1H), 7.11- | | | |
| | 7.07 (m, 3H), 7.00 (d, J = 8.5 Hz, 1H), 6.84- | | | |
| | 6.78 (m, 2H), 6.64-6.56 (m, 2H), 6.55- | | | |
| | 6.41 (m, 3H), 6.25 (d, J = 13.6 Hz, 1H), 5.07- | | | |
| | 4.99 (m, 1H), 4.47-4.40 (m, 1H), 4.38- | | | |
| | 4.30 (m, 1H), 4.30-4.20 (m, 1H), 4.13- | | | |
| | 4.04 (m, 1H), 4.00-3.89 (m, 1H), 3.84- | | | |
| | 3.76 (m, 1H), 3.19-3.09 (m, 2H), 3.06- | | | |
| | 2.82 (m, 10H), 2.75-2.67 (m, 1H), 2.63- | | | |
| | 2.54 (m, 1H), 2.43-2.36 (m, 3H), 2.22- | | | |
| | 2.05 (m, 2H), 1.98-1.88 (m, 3H), 1.79- | | | |
| | 1.58 (m, 4H), 1.52-1.38 (m, 4H). | | | |
| A148 | 1H NMR (400 MHz, DMSO) δ 10.97 (s, 1H), | 809.4 | 809.4 | |
| | 9.13 (s, 1H), 7.38 (d, J = 7.4 Hz, 1H), 7.26 (d, | | | |
| | J = 7.6 Hz, 1H), 7.13-7.06 (m, 3H), 6.84- | | | |
| | 6.79 (m, 2H), 6.64-6.57 (m, 2H), 6.55- | | | |
| | 6.42 (m, 3H), 6.26 (d, J = 13.6 Hz, 1H), 5.12- | | | |
| | 5.04 (m, 1H), 4.56-4.48 (m, 2H), 4.47- | | | |
| | 4.42 (m, 1H), 4.41-4.33 (m, 1H), 4.24- | | | |
| | 4.16 (m, 1H), 3.04-2.80 (m, 10H), 2.68- | | | |
| | 2.55 (m, 2H), 2.47-2.36 (m, 3H), 2.23- | | | |
| | 2.13 (m, 1H), 2.09-1.83 (m, 7H), 1.74- | | | |
| | 1.56 (m, 5H), 1.54-1.36 (m, 4H). | | | |
| A149 | 1H NMR (400 MHz, DMSO) δ 10.93 (s, 1H), | 810.4 | 810.4 | |
| | 9.24-9.09 (m, 1H), 7.15-7.00 (m, 4H), | | | |
| | 6.93 (s, 1H), 6.85-6.76 (m, 2H), 6.64-6.57 | | | |
| | (m, 2H), 6.56-6.41 (m, 3H), 6.31-6.21 (m, | | | |
| | 1H), 5.07-4.98 (m, 1H), 4.44 (d, J = 5.2 Hz, | | | |
| | 1H), 4.32-4.09 (m, 3H), 3.92-3.75 (m, | | | |
| | 2H), 3.18-3.08 (m, 2H), 3.07-2.81 (m, | | | |
| | 10H), 2.79-2.63 (m, 1H), 2.63-2.56 (m, | | | |
| | 1H), 2.43-2.27 (m, 3H), 2.25-2.03 (m, | | | |
| | 2H), 2.02-1.86 (m, 3H), 1.76-1.55 (m, | | | |
| | 4H), 1.52-1.34 (m, 4H). | | | |
| A150 | ¹H NMR (400 MHz, DMSO) δ 10.93 (s, 1H), | 740.6 | 740.6 | |
| | 9.15 (s, 1H), 7.12-6.78 (m, 7H), 6.64-6.44 | | | |
| | (m, 5H), 6.25 (d, J = 14.0 Hz, 1H), 5.02 (d, J = | | | |
| | 12.8 Hz, 1H), 4.44 (s, 1H), 4.32-4.09 (m, | | | |
| | 3H), 3.93-3.75 (m, 2H), 3.03-2.86 (m, | | | |
| | 10H), 2.79-2.70 (m, 2H), 2.43-2.30 (m, | | | |
| | 4H), 2.22-2.05 (m, 3H), 1.92 (s, 3H), 1.73- | | | |
| | 1.58 (m, 4H), 1.51-1.38 (m, 4H). | | | |
| A151 | ¹H NMR (400 MHz, MeOD) δ 7.16 (s, 1H), | 810.4 | 810.3 | |
| | 7.12 (s, 1H), 7.09-7.03 (m, 3H), 6.84-6.77 | | | |
| | (m, 2H), 6.63 (dd, J = 8.8, 5.4 Hz, 2H), 6.50 | | | |
| | (ddd, J = 8.2, 5.4, 2.7 Hz, 2H), 6.07 (dd, J = | | | |
| | 8.4, 2.1 Hz, 1H), 5.75 (dd, J = 12.0, 2.2 Hz, | | | |
| | 1H), 5.08 (dd, J = 13.3, 5.1 Hz, 1H), 4.52 (d, | | | |
| | J = 5.2 Hz, 1H), 4.36 (d, J = 2.4 Hz, 3H), 4.15 | | | |
| | (s, 1H), 4.10-4.01 (m, 1H), 3.55 (d, J = 27.4 | | | |
| | Hz, 3H), 3.49 (d, J = 7.2 Hz, 2H), 3.42 (t, J = | | | |
| | 4.7 Hz, 2H), 3.21 (d, J = 7.3 Hz, 1H), 3.03- | | | |
| | 2.94 (m, 3H), 2.93-2.83 (m, 2H), 2.81- | | | |
| | 2.76 (m, 1H), 2.51-2.38 (m, 1H), 2.35- | | | |
| | 2.24 (m, 1H), 2.23-2.08 (m, 2H), 1.99 (d, J = | | | |
| | 13.1 Hz, 3H), 1.78 (dd, J = 29.6, 13.5 Hz, | | | |
| | 4H), 1.59 (t, J = 11.9 Hz, 3H), 1.15 (dd, J = | | | |
| | 24.1, 10.5 Hz, 2H) | | | |
| A152 | ¹H NMR (400 MHz, MeOD) δ 7.10 (s, 1H), | 810.4 | 810.3 | |
| | 7.08-7.04 (m, 3H), 7.02 (s, 1H), 6.80 (dd, J = | | | |
| | 6.3, 3.1 Hz, 2H), 6.63 (dd, J = 10.7, 5.4 Hz, | | | |
| | 2H), 6.50 (t, J = 8.6 Hz, 2H), 6.06 (dd, J = | | | |
| | 8.4, 2.2 Hz, 1H), 5.74 (dd, J = 12.0, 2.1 Hz, | | | |
| | 1H), 5.07 (dd, J = 13.3, 5.1 Hz, 1H), 4.52 (d, | | | |
| | J = 5.3 Hz, 1H), 4.33 (d, J = 2.0 Hz, 2H), 4.29 | | | |
| | (dd, J = 10.8, 2.7 Hz, 1H), 3.97 (dd, J = 10.8, | | | |
| | 8.4 Hz, 1H), 3.90 (d, J = 12.3 Hz, 1H), 3.52- | | | |
| | 3.45 (m, 2H), 3.41 (q, J = 7.1 Hz, 2H), 3.13 | | | |
| | (dd, J = 27.4, 10.6 Hz, 2H), 3.02-2.91 (m, | | | |
| | 3H), 2.86 (dd, J = 13.2, 5.2 Hz, 1H), 2.81- | | | |
| | 2.71 (m, 1H), 2.52-2.36 (m, 4H), 2.34- | | | |
| | 2.23 (m, 1H), 2.22-2.09 (m, 2H), 2.06- | | | |
| | 2.00 (m, 1H), 1.94 (d, J = 12.5 Hz, 2H), 1.84- | | | |
| | 1.71 (m, 3H), 1.68-1.57 (m, 2H), 1.53 (t, J = | | | |
| | 12.0 Hz, 2H), 1.05 (dd, J = 21.7, 12.0 Hz, 2H). | | | |
| A153 | 1H NMR (400 MHz, DMSO) δ 10.97 (s, 1H), | 793.4 | 793.4 | |
| | 8.26 (s, 1H), 7.44-7.36 (m, 1H), 7.26-7.15 | | | |
| | (m, 6H), 6.95 (d, J = 7.2 Hz, 2H), 6.69 (d, J = | | | |
| | 8.4 Hz, 1H), 6.61 (d, J = 2.0 Hz, 1H), 6.54- | | | |
| | 6.48 (m, 1H), 5.13-5.04 (m, 1H), 4.50 (t, J = | | | |
| | 10.2 Hz, 2H), 4.40-4.34 (m, 1H), 4.24- | | | |
| | 4.17 (m, 1H), 4.12 (d, J = 4.8 Hz, 1H), 3.57 | | | |
| | (s, 2H), 3.52 (s, 2H), 3.36-3.29 (m, 2H), | | | |
| | 3.00-2.86 (m, 3H), 2.82-2.75 (m, 2H), | | | |
| | 2.66-2.54 (m, 1H), 2.47-2.34 (m, 1H), | | | |
| | 2.10 (d, J = 6.8 Hz, 2H), 2.00-1.86 (m, 6H), | | | |
| | 1.84-1.75 (m, 3H), 1.72-1.61 (m, 4H), | | | |
| | 1.51-1.36 (m, 3H), 0.95-0.82 (m, 2H). | | | |
| A154 | 1H NMR (400 MHz, DMSO) δ 10.97 (s, 1H), | 793.4 | 793.4 | |
| | 8.30 (s, 1H), 7.39 (d, J = 7.6 Hz, 1H), 7.28- | | | |
| | 7.15 (m, 6H), 6.95 (d, J = 7.2 Hz, 2H), 6.69 | | | |
| | (d, J = 8.4 Hz, 1H), 6.61 (s, 1H), 6.53-6.48 | | | |
| | (m, 1H), 5.12-5.05 (m, 1H), 4.54-4.48 (m, | | | |
| | 2H), 4.41-4.34 (m, 1H), 4.24-4.17 (m, | | | |
| | 1H), 4.12 (d, J = 4.8 Hz, 1H), 3.57 (s, 2H), | | | |
| | 3.52 (s, 2H), 3.37-3.31 (m, 2H), 3.01-2.87 | | | |
| | (m, 3H), 2.82-2.76 (m, 2H), 2.63-2.53 (m, | | | |
| | 1H), 2.44-2.33 (m, 1H), 2.10 (d, J = 6.8 Hz, | | | |
| | 2H), 2.01-1.86 (m, 6H), 1.81 (d, J = 12.0 | | | |
| | Hz, 3H), 1.72-1.62 (m, 4H), 1.52-1.35 (m, | | | |
| | 3H), 0.98-0.83 (m, 2H). | | | |
| A155 | 1H NMR (400 MHz, DMSO) δ 10.93 (s, 1H), | 794.4 | 794.4 | |
| | 7.28-7.15 (m, 6H), 7.02-6.91 (m, 3H), | | | |
| | 6.68 (d, J = 8.4 Hz, 1H), 6.61 (d, J = 2.0 Hz, | | | |
| | 1H), 6.53-6.46 (m, 1H), 5.08-4.96 (m, | | | |
| | 1H), 4.39-4.33 (m, 1H), 4.29-4.21 (m, | | | |
| | 1H), 4.15-4.06 (m, 2H), 3.99-3.91 (m, | | | |
| | 1H), 3.81 (d, J = 10.8 Hz, 1H), 3.56-3.51 | | | |
| | (m, 3H), 3.36-3.30 (m, 1H), 3.19-3.14 (m, | | | |
| | 1H), 3.00-2.84 (m, 5H), 2.76-2.59 (m, | | | |
| | 2H), 2.59-2.53 (m, 1H), 2.43-2.34 (m, | | | |
| | 1H), 2.17-2.02 (m, 3H), 1.99-1.91 (m, | | | |
| | 2H), 1.83-1.76 (m, 3H), 1.73-1.62 (m, | | | |
| | 3H), 1.53-1.36 (m, 3H), 1.01 (t, J = 7.2 Hz, | | | |
| | 1H), 0.97-0.82 (m, 2H). | | | |
| A156 | 1H NMR (400 MHz, DMSO) δ 11.00 (s, 1H), | 794.4 | 794.4 | |
| | 7.31-7.22 (m, 6H), 7.07-6.94 (m, 3H), | | | |
| | 6.74 (d, J = 8.4 Hz, 1H), 6.67 (d, J = 2.0 Hz, | | | |
| | 1H), 6.59-6.53 (m, 1H), 5.13-5.03 (m, | | | |
| | 1H), 4.45-4.37 (m, 1H), 4.34-4.27 (m, | | | |
| | 1H), 4.22-4.11 (m, 2H), 4.05-3.98 (m, | | | |
| | 1H), 3.89-3.84 (m, 1H), 3.63-3.60 (m, | | | |
| | 2H), 3.58 (s, 3H), 3.24-3.19 (m, 1H), 3.13- | | | |
| | 2.86 (m, 6H), 2.79 (t, J = 10.4 Hz, 1H), 2.68- | | | |
| | 2.58 (m, 1H), 2.51-2.38 (m, 1H), 2.24- | | | |
| | 2.08 (m, 3H), 2.05-1.95 (m, 2H), 1.89- | | | |
| | 1.81 (m, 3H), 1.79-1.69 (m, 3H), 1.58- | | | |
| | 1.42 (m, 3H), 1.03-0.87 (m, 2H). | | | |
| A157 | ¹H NMR (400 MHz, DMSO) δ 10.94 (s, 1H), | 794.4 | 794.4 | |
| | 7.26-7.15 (m, 5H), 7.03 (s, 1H), 6.94 (d, J = | | | |
| | 6.6 Hz, 3H), 6.68 (d, J = 8.4 Hz, 1H), 6.61 (s, | | | |
| | 1H), 6.55-6.47 (m, 1H), 5.06-4.97 (m, | | | |
| | 1H), 4.32-4.20 (m, 2H), 4.17-4.07 (m, | | | |
| | 2H), 3.97-3.82 (m, 2H), 3.81-3.72 (m, | | | |
| | 2H), 3.37-3.30 (m, 2H), 3.19-3.01 (m, | | | |
| | 2H), 3.02-2.80 (m, 6H), 2.75 (t, J = 10.4 Hz, | | | |
| | 1H), 2.61-2.54 (m, 1H), 2.41-2.28 (m, | | | |
| | 1H), 2.16-2.03 (m, 3H), 2.00-1.90 (m, | | | |
| | 2H), 1.84-1.74 (m, 3H), 1.72-1.61 (m, | | | |
| | 3H), 1.53-1.36 (m, 3H), 0.98-0.84 (m, 2H). | | | |
| A158 | ¹H NMR (400 MHz, DMSO) δ 10.93 (s, 1H), | 794.4 | 794.4 | |
| | 8.31 (s, 1H), 7.25-7.12 (m, 5H), 7.03 (s, | | | |
| | 1H), 6.97-6.88 (m, 3H), 6.68 (d, J = 8.4 Hz, | | | |
| | 1H), 6.61 (s, 1H), 6.53-6.44 (m, 1H), 5.06- | | | |
| | 4.98 (m, 1H), 4.33-4.19 (m, 2H), 4.18 | | | |
| | 4.08 (m, 2H), 3.92-3.75 (m, 3H), 3.58- | | | |
| | 3.55 (m, 2H), 3.36-3.32 (m, 1H), 3.17- | | | |
| | 3.12 (m, 1H), 3.00-2.85 (m, 5H), 2.79- | | | |
| | 2.70 (m, 1H), 2.66-2.51 (m, 2H), 2.40- | | | |
| | 2.30 (m, 1H), 2.19-2.02 (m, 3H), 1.99- | | | |
| | 1.89 (m, 2H), 1.84-1.75 (m, 3H), 1.74- | | | |
| | 1.63 (m, 3H), 1.53-1.36 (m, 3H), 1.06- | | | |
| | 0.97 (m, 1H), 0.95-0.83 (m, 2H). | | | |
| A159 | ¹H NMR (400 M Hz, MeOD) δ 10.92 (s, | 810.4 | 810.3 | |
| | 1H), 9.11 (s, 1H), 7.17 (d, J = 8.4 Hz, 1H), | | | |
| | 7.11-7.08 (m, 3H), 7.01 (d, J = 8.1 Hz, 1H), | | | |
| | 6.82-6.80 (m, 2H), 6.60-6.58 (m, 2H), 6.48- | | | |
| | 6.40 (m, 2H), 6.01 (d, J = 10.0 Hz, 1H), 5.74 | | | |
| | (d, J = 12.0 Hz, 1H), 5.05-5.00 (m, 1H), 4.43 | | | |
| | (d, J = 4.8 Hz, 2H), 4.37-4.34 (m, 1H), 4.25 | | | |
| | (d, J = 17.2 Hz, 1H), 4.10 (d, J = 17.2 Hz, 1H), | | | |
| | 3.98-3.92 (m, 1H), 3.83-3.79 (m, 1H), 3.41 (s, | | | |
| | 2H), 3.35 (s, 2H), 3.25-3.18 (m, 1H), 2.99- | | | |
| | 2.85 (m, 5H), 2.73-2.67 (m, 1H), 2.40-2.33 | | | |
| | (m, 1H), 2.19-2.08 (m, 4H), 2.07-2.00 (m, | | | |
| | 2H), 1.84-1.81 (m, 2H), 1.72-1.66 (m, 4H), | | | |
| | 1.49-1.39 (m, 3H), 1.23 (s, 1H) | | | |
| A160 | ¹H NMR (400 MHz, DMSO-d₆) δ 10.97 (s, | 809.4 | 809.5 | |
| | 1H), 9.14 (s, 1H), 7.39-7.37 (m, 1H), 7.30- | | | |
| | 7.28(m, 1H), 7.14-7.10 (m, 3H), 6.84-6.81 | | | |
| | (m, 2H), 6.61-6.59 (m, 2H), 6.50-6.42 (m, | | | |
| | 2H), 6.04-6.01 (m, 1H), 5.77-5.73 (m, 1H), | | | |
| | 5.11-5.06 (m, 1H) , 4.55 (s, 2H) , 4.44-4.36 | | | |
| | (m, 2H), 4.20 (d, J = 13.2 Hz, 1H), 3.00-2.86 | | | |
| | (m, 5H), 2.62-2.57 (m, 1H), 2.44-2.40 (m, | | | |
| | 2H), 2.20-2.16 (m, 2H), 2.08-1.96 (m, 4H), | | | |
| | 1.87-1.83 (m, 2H), 1.72-1.69 (m, 5H), 1.48- | | | |
| | 1.42 (m, 3H), 1.24 (s, 6H), 0.96-0.93 (m, 2H) | | | |
| A161 | ¹H NMR (400 MHz, DMSO-d₆) δ 10.97 (s, | 809.4 | 809.5 | |
| | 1H), 9.16 (s, 1H), 7.40-7.37 (m, 1H), 7.28- | | | |
| | 7.26(m, 1H), 7.11-7.10 (m, 3H), 6.83-6.81 | | | |
| | (m, 2H), 6.62-6.59 (m, 2H), 6.50-6.42 (m, | | | |
| | 2H), 6.04-6.01 (m, 1H), 5.76-5.73 (m, 1H), | | | |
| | 5.11-5.06 (m, 1H) , 4.52 (s, 2H) , 4.44-4.36 | | | |
| | (m, 2H), 4.24-4.19 (m, 1H), 2.95-2.80 (m, | | | |
| | 5H), 2.67-2.61 (m, 1H), 2.44-2.33 (m, 2H), | | | |
| | 2.12-2.11 (m, 2H), 2.02-1.96 (m, 4H), 1.85- | | | |
| | 1.82 (m, 2H), 1.70-1.67 (m, 5H), 1.46- | | | |
| | 1.40 (m, 3H), 1.24 (s, 6H), 0.92-0.86 (m, 2H) | | | |
| A162 | ¹H NMR (400 MHz, MeOD) δ 7.37 (s, 1H), | 821.4 | 821.5 | |
| | 7.15 (d, J = 7.6 Hz, 1H), 7.07-7.03 (m, 3H), | | | |
| | 6.80 (s, 1H), 6.74 (dd, J = 6.3, 2.9 Hz, 3H), | | | |
| | 6.64 (d, J = 2.2 Hz, 1H), 6.58 (d, J = 8.4 Hz, | | | |
| | 2H), 6.49 (dd, J = 8.4, 2.5 Hz, 1H), 5.12 (dd, | | | |
| | J = 13.2, 5.1 Hz, 1H), 4.62 (s, 2H), 4.43 (t, J = | | | |
| | 13.7 Hz, 2H), 3.65-3.54 (m, 3H), 3.39 (s, | | | |
| | 2H), 3.10 (s, 5H), 2.99 (dd, J = 9.7, 5.5 Hz, | | | |
| | 2H), 2.88 (dd, J = 12.7, 4.7 Hz, 1H), 2.80 (dd, | | | |
| | J = 6.5, 5.0 Hz, 1H), 2.47 (dd, J = 13.2, 4.6 | | | |
| | Hz, 1H), 2.32-2.21 (m, 6H), 2.06 (dd, J = | | | |
| | 21.6, 10.0 Hz, 5H), 1.89 (s, 2H), 1.83-1.75 | | | |
| | (m, 2H), 1.70 (d, J = 9.8 Hz, 1H), 1.31 (d, J = | | | |
| | 18.0 Hz, 5H) | | | |
| A163 | ¹H NMR (400 MHz, DMSO) δ 10.92 (s, 1H), | 840.4 | 840.4 | |
| | 9.11 (s, 1H), 7.22-6.95 (m, 5H), 6.78 (d, J = | | | |
| | 6.8 Hz, 2H), 6.65-6.39 (m, 3H), 6.10 (d, J = | | | |
| | 13.6 Hz, 1H), 5.67 (d, J = 8.0 Hz, 1H), 5.03 | | | |
| | (dd, J = 13.2, 5.2 Hz, 1H), 4.62 (d, J = 5.2 Hz, | | | |
| | 1H), 4.40-4.31 (m, 1H), 4.18 (dd, J = 62.8, | | | |
| | 16.8 Hz, 2H), 4.01-3.89 (m, 1H), 3.81 (d, J = | | | |
| | 11.6 Hz, 1H), 3.57-3.45 (m, 4H), 3.23- | | | |
| | 3.14 (m, 2H), 3.02-2.82 (m, 8H), 2.74 (d, J = | | | |
| | 10.4 Hz, 1H), 2.62-2.54 (m, 1H), 2.43- | | | |
| | 2.31 (m, 1H), 2.20-2.01 (m, 4H), 1.99- | | | |
| | 1.84 (m, 3H), 1.76-1.58 (m, 4H), 1.55- | | | |
| | 1.37 (m, 3H), 1.00-0.82 (m, 2H). | | | |
| A164 | ¹H NMR (400 MHz, DMSO) δ 10.92 (s, 1H), | 840.4 | 840.4 | |
| | 9.11 (s, 1H), 7.14-7.00 (m, 4H), 6.93 (s, | | | |
| | 1H), 6.78 (d, J = 6.8 Hz, 2H), 6.60-6.43 (m, | | | |
| | 3H), 6.10 (d, J = 13.6 Hz, 1H), 5.67 (d, J = | | | |
| | 8.0 Hz, 1H), 5.02 (dd, J = 13.2, 4.8 Hz, 1H), | | | |
| | 4.62 (d, J = 5.2 Hz, 1H), 4.32-4.11 (m, 3H), | | | |
| | 3.93-3.76 (m, 2H), 3.57-3.45 (m, 4H), | | | |
| | 3.24-3.14 (m, 2H), 2.98-2.84 (m, 8H), | | | |
| | 2.80-2.72 (m, 1H), 2.64-2.57 (m, 1H), | | | |
| | 2.39-2.30 (m, 1H), 2.17-2.07 (m, 3H), | | | |
| | 1.98-1.84 (m, 3H), 1.75-1.59 (m, 4H), | | | |
| | 1.54-1.38 (m, 3H), 1.24 (s, 1H), 0.98-0.85 | | | |
| | (m, 2H). | | | |
| A165 | ¹H NMR (400 MHz, DMSO) δ 10.92 (s, 1H), | 840.4 | 840.4 | |
| | 7.19-6.97 (m, 5H), 6.78 (d, J = 6.8 Hz, 2H), | | | |
| | 6.64-6.46 (m, 3H), 6.10 (d, J = 13.6 Hz, | | | |
| | 1H), 5.67 (d, J = 8.0 Hz, 1H), 5.07-4.93 (m, | | | |
| | 1H), 4.62 (d, J = 5.2 Hz, 1H), 4.40-4.34 (m, | | | |
| | 1H), 4.31-4.09 (m, 2H), 3.97 (d, J = 8.8 Hz, | | | |
| | 1H), 3.86-3.79 (m, 1H), 3.62-3.46 (m, | | | |
| | 5H), 3.29-3.07 (m, 5H), 2.91 (d, J = 10.6 | | | |
| | Hz, 6H), 2.59 (s, 1H), 2.42-2.34 (m, 1H), | | | |
| | 2.17-1.88 (m, 6H), 1.72-1.36 (m, 7H), | | | |
| | 1.24 (s, 1H), 0.98-0.84 (m, 2H). | | | |
| A166 | 1H NMR (400 MHz, DMSO-d6) δ 10.96 (s, 1 | 840.4 | 840.4 | |
| | H), 8.26 (s, 1 H), 7.45 (s, 1 H), 7.12-7.02 (m, | | | |
| | 3 H), 7.00 (s, 1 H), 6.78 (d, J = 6.8 Hz, 2 H), | | | |
| | 6.58-6.54 (m, 2 H), 6.49-6.46 (m, 1 H), 6.10 | | | |
| | (d, J = 13.6 Hz, 1 H), 5.67 (d, J = 8.4 Hz, 1 H), | | | |
| | 5.09-5.05 (m, 1 H), 4.63-4.62 (m, 1 H), 4.55 | | | |
| | (s, 2 H), 4.34 (d, J = 16.8 Hz, 1 H), 4.21 (d, | | | |
| | J = 17.2 Hz, 1 H), 3.57-3.46 (m, 4 H), 3.24- | | | |
| | 3.20 (m, 2 H), 2.93-2.78 (m, 8 H), 2.63-2.57 | | | |
| | (m, 1 H), 2.43-2.31 (m, 1 H), 2.18-2.09 (m, 2 | | | |
| | H), 2.00-1.86 (m, 7 H), 1.71-1.60 (m, 5 H), | | | |
| | 1.52-1.40 (m, 3 H), 0.97-0.85 (m, 2 H). | | | |
| A167 | 1H NMR (400 MHz, DMSO) δ 10.96 (s, 1H), | 839.4 | 839.5 | |
| | 7.45 (s, 1H), 7.14-7.03 (m, 3H), 7.00 (s, | | | |
| | 1H), 6.78 (d, J = 6.8 Hz, 2H), 6.59-6.43 (m, | | | |
| | 3H), 6.10 (d, J = 13.6 Hz, 1H), 5.68 (d, J = | | | |
| | 8.0 Hz, 1H), 5.07 (dd, J = 13.2, 5.2 Hz, 1H), | | | |
| | 4.62 (d, J = 4.8 Hz, 1H), 4.48-4.41 (m, 2H), | | | |
| | 4.27 (dd, J = 52.4, 17.2 Hz, 2H), 3.56-3.46 | | | |
| | (m, 5H), 3.26-3.19 (m, 2H), 2.95-2.77 (m, | | | |
| | 8H), 2.62-2.56 (m, 1H), 2.40-2.31 (m, | | | |
| | 1H), 2.14-2.08 (m, 2H), 1.98-1.84 (m, | | | |
| | 7H), 1.74-1.61 (m, 5H), 1.51-1.38 (m, | | | |
| | 3H), 0.96-0.85 (m, 2H). | | | |
| A168 | 1H NMR (400 MHz, DMSO) δ 10.96 (s, 1H), | 839.4 | 839.5 | |
| | 9.56-8.58 (m, 1H), 7.39 (d, J = 7.6 Hz, 1H), | | | |
| | 7.26 (d, J = 7.6 Hz, 1H), 7.13-7.05 (m, 3H), | | | |
| | 6.78 (d, J = 7.2 Hz, 2H), 6.59-6.53 (m, 2H), | | | |
| | 6.50-6.45 (m, 1H), 6.10 (d, J = 13.2 Hz, | | | |
| | 1H), 5.68 (d, J = 8.0 Hz, 1H), 5.08 (dd, J = | | | |
| | 13.2, 4.8 Hz, 1H), 4.62 (d, J = 4.8 Hz, 1H), | | | |
| | 4.51 (s, 2H), 4.38 (d, J = 17.2 Hz, 1H), 4.21 | | | |
| | (d, J = 17.2 Hz, 1H), 3.22 (d, J = 14.4 Hz, | | | |
| | 2H), 2.93 (s, 3H), 2.84-2.78 (m, 2H), 2.69- | | | |
| | 2.55 (m, 2H), 2.49-2.34 (m, 2H), 2.25- | | | |
| | 2.04 (m, 4H), 2.00-1.84 (m, 8H), 1.76- | | | |
| | 1.58 (m, 6H), 1.56-1.33 (m, 4H), 1.25- | | | |
| | 1.15 (m, 1H), 0.95-0.85 (m, 2H). | | | |
| A169 | 1H NMR (400 MHz, DMSO) δ 10.93 (s, 1H), | 840.4 | 840.5 | |
| | 8.37 (s, 1H), 7.12-7.02 (m, 4H), 6.93 (s, | | | |
| | 1H), 6.78 (d, J = 6.8 Hz, 2H), 6.60-6.53 (m, | | | |
| | 2H), 6.48 (d, J = 8.4 Hz, 1H), 6.10 (d, J = | | | |
| | 13.6 Hz, 1H), 5.67 (d, J = 8.0 Hz, 1H), 5.02 | | | |
| | (dd, J = 13.2, 5.2 Hz, 1H), 4.62 (d, J = 4.8 Hz, | | | |
| | 1H), 4.32-4.11 (m, 3H), 3.94-3.77 (m, | | | |
| | 2H), 3.55-3.48 (m, 4H), 3.28-3.13 (m, | | | |
| | 4H), 2.92 (s, 3H), 2.90-2.72 (m, 4H), 2.68- | | | |
| | 2.52 (m, 2H), 2.38-1.97 (m, 6H), 1.87 (d, J = | | | |
| | 11.6 Hz, 2H), 1.74-1.59 (m, 4H), 1.47- | | | |
| | 1.39 (m, 2H), 0.97-0.85 (m, 2H). | | | |
| A170 | 1H NMR (400 MHz, DMSO) δ 10.94 (s, 1H), | 800.4 | 800.4 | |
| | 7.15-7.02 (m, 4H), 6.94 (s, 1H), 6.77 (d, J = | | | |
| | 6.6 Hz, 2H), 6.61-6.53 (m, 3H), 6.49-6.37 | | | |
| | (m, 2H), 5.10-4.96 (m, 1H), 4.60 (d, J = 5.2 | | | |
| | Hz, 1H), 4.33-4.10 (m, 3H), 3.93-3.76 (m, | | | |
| | 2H), 3.31-3.12 (m, 5H), 3.02-2.85 (m, | | | |
| | 8H), 2.82-2.73 (m, 1H), 2.64-2.52 (m, | | | |
| | 3H), 2.41-2.16 (m, 4H), 2.14-2.05 (m, | | | |
| | 1H), 2.00-1.91 (m, 1H), 1.84-1.59 (m, | | | |
| | 5H), 1.32-1.17 (m, 2H). | | | |
| A171 | 1H NMR (400 MHz, DMSO) δ 10.94 (s, 1H), | 800.4 | 800.4 | |
| | 7.14-7.01 (m, 4H), 6.94 (s, 1H), 6.77 (d, J = | | | |
| | 6.6 Hz, 2H), 6.61-6.53 (m, 3H), 6.49-6.38 | | | |
| | (m, 2H), 5.08-4.96 (m, 1H), 4.60 (d, J = 5.4 | | | |
| | Hz, 1H), 4.34-4.08 (m, 3H), 3.95-3.75 (m, | | | |
| | 2H), 3.31-3.13 (m, 5H), 3.03-2.84 (m, | | | |
| | 8H), 2.82-2.73 (m, 1H), 2.63-2.52 (m, | | | |
| | 3H), 2.41-2.15 (m, 4H), 2.13-2.03 (m, | | | |
| | 1H), 2.01-1.91 (m, 1H), 1.84-1.58 (m, | | | |
| | 5H), 1.32-1.16 (m, 2H). | | | |
| A172 | 1H NMR (400 MHz, DMSO) δ 10.96 (s, 1H), | 839.4 | 839.6 | |
| | 8.32 (s, 1H), 7.45 (s, 1H), 7.16-6.96 (m, | | | |
| | 5H), 6.78 (d, J = 6.8 Hz, 2H), 6.58-6.46 (m, | | | |
| | 3H), 6.10 (d, J = 13.6 Hz, 1H), 5.68 (d, J = | | | |
| | 8.0 Hz, 1H), 5.07 (dd, J = 13.2, 4.8 Hz, 1H), | | | |
| | 4.62 (d, J = 4.8 Hz, 1H), 4.45 (s, 2H), 4.34 (d, | | | |
| | J = 17.2 Hz, 1H), 4.21 (d, J = 16.8 Hz, 1H), | | | |
| | 2.93 (s, 3H), 2.83-2.78 (m, 2H), 2.70-2.54 | | | |
| | (m, 2H), 2.48-2.24 (m, 2H), 2.21-2.06 (m, | | | |
| | 4H), 2.00-1.85 (m, 8H), 1.73-1.59 (m, | | | |
| | 6H), 1.54-1.34 (m, 4H), 0.95-0.87 (m, 2H). | | | |
| A173 | ¹H NMR (400 MHz, DMSO-d6) 10.93 (br s, | 856.4 | 856.3 | |
| | 1H), 9.15 (br s, 1H), 7.13-7.02 (m, 4H), 6.94 | | | |
| | (s, 1H), 6.78-6.72 (m, 2H), 6.61-6.53 (m, | | | |
| | 2H), 6.48 (dd, J = 2.3, 8.4 Hz, 1H), 6.24-6.14 | | | |
| | (m, 2H), 5.03 (dd, J = 5.2, 13.3 Hz, 1H), 4.67 | | | |
| | (br d, J = 5.1 Hz, 1H), 4.32-4.22 (m, 2H), | | | |
| | 4.19-4.11 (m, 1H), 3.95-3.86 (m, 2H), 3.82 | | | |
| | (br d, J = 11.6 Hz, 1H), 3.19 (br d, J = 9.1 Hz, | | | |
| | 2H), 3.05-2.84 (m, 13H), 2.81-2.73 (m, | | | |
| | 1H), 2.62-2.54 (m, 2H), 2.41-2.30 (m, 3H), | | | |
| | 2.21-2.05 (m, 2H), 1.97 (br dd, J = 7.9, 11.8 | | | |
| | Hz, 2H), 1.77-1.57 (m, 6H), 1.37 (br dd, | | | |
| | J = 8.3, 12.3 Hz, 1H). | | | |
| A174 | ¹H NMR (400 MHz, DMSO-d6) 10.94 (br s, | 856.4 | 856.3 | |
| | 1H), 9.16 (br s, 1H), 7.18 (d, J = 8.3 Hz, 1H), | | | |
| | 7.14-6.98 (m, 4H), 6.75 (br d, J = 6.3 Hz, | | | |
| | 2H), 6.63-6.54 (m, 2H), 6.52-6.44 (m, 1H), | | | |
| | 6.28-6.14 (m, 2H), 5.03 (dd, J = 5.0, 13.3 Hz, | | | |
| | 1H), 4.67 (br d, J = 5.1 Hz, 1H), 4.36 (br d, | | | |
| | J = 9.3 Hz, 1H), 4.31-4.19 (m, 1H), 4.11 (br | | | |
| | d, J = 16.9 Hz, 1H), 4.01-3.78 (m, 3H), 3.19 | | | |
| | (br d, J = 8.5 Hz, 3H), 3.05-2.83 (m, 13H), | | | |
| | 2.80-2.71 (m, 1H), 2.63-2.55 (m, 2H), 2.41- | | | |
| | 2.27 (m, 2H), 2.13 (br t, J = 11.3 Hz, 2H), | | | |
| | 1.96 (br dd, J = 7.3, 12.0 Hz, 2H), 1.77-1.56 | | | |
| | (m, 6H), 1.37 (br dd, J = 8.3, 12.4 Hz, 1H). | | | |
| A175 | ¹H NMR (400 MHz, DMSO-d6) 10.97 (br s, | 855.4 | 855.3 | |
| | 1H), 9.74-8.67 (m, 1H), 7.46 (br s, 1H), 7.20- | | | |
| | 6.99 (m, 4H), 6.76 (br d, J = 5.8 Hz, 2H), | | | |
| | 6.65-6.40 (m, 3H), 6.20 (br d, J = 10.3 Hz, | | | |
| | 2H), 5.08 (br d, J = 8.8 Hz, 1H), 4.68 (br s, | | | |
| | 1H), 4.46 (br s, 2H), 4.37-4.30 (m, 1H), 4.28- | | | |
| | 4.14 (m, 1H), 3.88 (br s, 1H), 3.28 (br s, | | | |
| | 1H), 3.05-2.78 (m, 12H), 2.59 (br d, J = 16.0 | | | |
| | Hz, 2H), 2.42-2.28 (m, 3H), 2.16 (br d, | | | |
| | J = 7.3 Hz, 1H), 2.08-1.82 (m, 7H), 1.66 (br s, | | | |
| | 7H), 1.38 (br d, J = 8.4 Hz, 1H). | | | |
| A176 | ¹H NMR (400 MHz, DMSO-d6) 10.98 (br s, | 855.4 | 855.3 | |
| | 1H), 9.54-8.54 (m, 1H), 7.48-7.20 (m, 2H), | | | |
| | 7.09 (br s, 3H), 6.76 (br d, J = 4.0 Hz, 2H), | | | |
| | 6.65-6.41 (m, 3H), 6.20 (br d, J = 9.8 Hz, | | | |
| | 2H), 5.09 (br d, J = 8.6 Hz, 1H), 4.68 (br s, | | | |
| | 1H), 4.52 (br s, 2H), 4.38 (br d, J = 17.0 Hz, | | | |
| | 1H), 4.22 (br d, J = 16.8 Hz, 1H), 3.89 (br s, | | | |
| | 1H), 3.28 (br s, 1H), 3.02-2.78 (m, 13H), | | | |
| | 2.59 (br d, J = 16.5 Hz, 2H), 2.45-2.29 (m, | | | |
| | 3H), 2.15 (br s, 1H), 1.97 (br d, J = 11.9 Hz, | | | |
| | 6H), 1.67 (br s, 7H), 1.37 (br s, 1H). | | | |
| A177 | ¹H NMR (400 MHz, MeOD) δ 7.43 (d, J = | 790.4 | 791.5 | |
| | 7.6 Hz, 1H), 7.34 (d, J = 8.1 Hz, 1H), 7.11 (d, | | | |
| | J = 6.8 Hz, 3H), 6.80 (d, J = 6.0 Hz, 2H), 6.68 | | | |
| | (d, J = 8.4 Hz, 1H), 6.64 (s, 1H), 6.51 (d, J = | | | |
| | 8.1 Hz, 1H), 6.25 (d, J = 8.3 Hz, 2H), 6.14 (d, | | | |
| | J = 8.4 Hz, 2H), 5.14 (dd, J = 13.2, 5.0 Hz, | | | |
| | 1H), 4.69 (s, 2H), 4.43 (q, J = 17.1 Hz, 2H), | | | |
| | 4.15 (d, J = 4.8 Hz, 1H), 3.67 (d, J = 11.4 Hz, | | | |
| | 2H), 3.49 (s, 2H), 3.43 (s, 2H), 3.17-2.95 | | | |
| | (m, 6H), 2.89 (dd, J = 21.9, 9.2 Hz, 1H), 2.78 | | | |
| | (d, J = 15.5 Hz, 1H), 2.50 (dt, J = 13.5, 8.6 | | | |
| | Hz, 1H), 2.28 (d, J = 13.0 Hz, 2H), 2.03 (t, J = | | | |
| | 22.0 Hz, 6H), 1.80 (d, J = 15.6 Hz, 3H), | | | |
| | 1.59 (t, J = 11.1 Hz, 4H), 1.18 (d, J = 12.0 | | | |
| | Hz, 2H). | | | |
| A178 | ¹H NMR (400 MHz, MeOD) δ 7.37 (s, 1H), | 790.4 | 791.5 | |
| | 7.13 (dd, J = 16.9, 7.4 Hz, 4H), 6.81 (d, J = | | | |
| | 7.4 Hz, 2H), 6.71-6.60 (m, 2H), 6.51 (d, J = | | | |
| | 8.3 Hz, 1H), 6.26 (d, J = 8.4 Hz, 2H), 6.16 (d, | | | |
| | J = 8.4 Hz, 2H), 5.12 (dd, J = 13.5, 5.3 Hz, | | | |
| | 1H), 4.62 (s, 2H), 4.42 (d, J = 7.7 Hz, 1H), | | | |
| | 4.16 (d, J = 4.7 Hz, 1H), 3.66 (d, J = 12.4 Hz, | | | |
| | 2H), 3.51 (s, 2H), 3.44 (s, 2H), 3.17-2.99 | | | |
| | (m, 6H), 2.88 (d, J = 12.8 Hz, 1H), 2.77 (d, J = | | | |
| | 14.4 Hz, 1H), 2.39-2.23 (m, 4H), 2.01 (d, | | | |
| | J = 13.1 Hz, 6H), 1.80 (d, J = 14.7 Hz, 3H), | | | |
| | 1.60 (t, J = 11.2 Hz, 4H), 1.18 (d, J = 11.2 | | | |
| | Hz, 2H). | | | |
| A179 | 1H NMR (400 MHz, DMSO) δ 10.83 (s, 1H), | 827.4 | 828.3 | |
| | 8.82 (d, J = 8.4 Hz, 1H), 8.27 (s, 1H), 7.87 (d, | | | |
| | J = 7.6 Hz, 1H), 7.60 (d, J = 7.6 Hz, 1H), 7.15- | | | |
| | 7.01 (m, 3H), 6.78 (d, J = 6.8 Hz, 2H), 6.61- | | | |
| | 6.52 (m, 2H), 6.48 (d, J = 8.0 Hz, 1H), 6.14- | | | |
| | 6.02 (m, 1H), 5.68 (d, J = 8.0 Hz, 1H), 4.80- | | | |
| | 4.70 (m, 1H), 4.62 (d, J = 4.8 Hz, 1H), 4.48 | | | |
| | (s, 2H), 3.55-3.49 (m, 3H), 3.27-3.17 (m, | | | |
| | 1H), 3.01-2.82 (m, 6H), 2.79-2.71 (m, | | | |
| | 3H), 2.23-2.08 (m, 4H), 2.03-1.79 (m, | | | |
| | 8H), 1.75-1.61 (m, 5H), 1.51-1.37 (m, | | | |
| | 3H), 1.00-0.85 (m, 2H). | | | |
| A180 | 1 H NMR (400 MHz, DMSO-d6) δ 10.92 (s, | 813.9 | 814.5 | |
| | 1 H), 7.18 (d, J = 8.4 Hz, 1 H), 7.14-6.98 (m, | | | |
| | 4 H), 6.75 (d, J = 6.0 Hz, 2 H), 6.60-6.54 (m, | | | |
| | 2 H), 6.51-6.45 (m, 1 H), 6.26-6.14 (m, 2 H), | | | |
| | 5.03 (dd, J = 13.2, 4.8 Hz, 1 H), 4.67 (d, J = | | | |
| | 5.6 Hz, 1 H), 4.36 (d, J = 8.0 Hz, 1 H), 4.30- | | | |
| | 4.07 (m, 2 H), 4.00-3.93 (m, 1 H), 3.82 (d, J = | | | |
| | 11.6 Hz, 1 H), 3.27-3.12 (m, 6 H), 3.01-2.84 | | | |
| | (m, 8 H), 2.78-2.67 (m, 1 H), 2.62-2.54 (m, 2 | | | |
| | H), 2.42-2.29 (m, 3 H), 2.19-2.04 (m, 2 H), | | | |
| | 1.98-1.89 (m, 1 H), 1.78-1.59 (m, 4 H), 1.48- | | | |
| | 1.22 (m, 5 H). | | | |
| A181 | 1H NMR (400 MHz, MeOD-d₄) 88.52 (s, | 790.4 | 791.4 | |
| | 1H), 7.40-7.33 (m, 2H), 7.10-7.08 (m, 3H), | | | |
| | 6.80-6.78 (m, 2H), 6.67(d, J = 8.4, 1H), 6.63- | | | |
| | 6.59 (m, 3H), 6.52-6.49 (m, 1H), 6.28 (d, J = | | | |
| | 8.4, 2H), 5.14-5.10 (m, 1H), 5.14-5.10 (m, | | | |
| | 1H), 4.58 (s, 2H), 4.46-4.35 (m, 2H), 4.16 | | | |
| | (d, J = 4.0, 1H), 3.12-3.09 (m, 2H), 3.10- | | | |
| | 2.97 (m, 4H), 2.89-2.86 (m, 3H), 2.78- | | | |
| | 2.74 (m, 3H), 2.64-2.42 (m, 4H) , 2.18- | | | |
| | 2.12 (m, 2H), 2.08-2.05 (m, 4H), 1.87- | | | |
| | 1.83 (m, 2H) , 1.76-1.73 (m, 3H), 1.64- | | | |
| | 1.60 (m, 2H) , 1.58-1.53 (m, 2H), 1.34- | | | |
| | 1.28 (m, 1H). | | | |
| A182 | 1H NMR (400 MHz, MeOD-d₄) 87.36 (s, | 790.4 | 791.4 | |
| | 1H), 7.15-7.08 (m, 4H), 6.80-6.78 (m, 2H), | | | |
| | 6.69-6.63 (m, 4H), 6.52-6.49 (m, 1H), 6.31 | | | |
| | (d, J = 8.8, 2H), 5.14-5.09 (m, 1H), 4.94- | | | |
| | 4.91 (m, 1H) , 4.63-4.60 (m, 2H), 4.49- | | | |
| | 4.36 (m, 2H), 4.18 (d, J = 4.8, 1H), 3.57- | | | |
| | 3.47 (m, 3H), 3.12-2.97 (m, 6H), 2.95- | | | |
| | 2.85 (m, 3H), 2.79-2.71 (m, 2H), 2.49-2.42 | | | |
| | (m, 1H) , 2.26-2.14 (m, 7H), 2.09-2.05 (m, | | | |
| | 2H), 1.82-1.80 (m, 2H) , 1.78-1.74 (m, | | | |
| | 1H), 1.69-1.64 (m, 4H). | | | |
| A183 | 1 H NMR (400 MHz, DMSO-d6) δ 10.96 (s, | 766.4 | 767.5 | |
| | 1 H), 7.44-7.32 (m, 1 H), 7.30-7.19 (m, 1 H), | | | |
| | 7.19-7.08 (m, 3 H), 6.82 (d, J = 6.8 Hz, 2 H), | | | |
| | 6.66-6.56 (m, 2 H), 6.54-6.39 (m, 1 H), 6.18 | | | |
| | (d, J = 8.4 Hz, 2 H), 6.06 (d, J = 8.4 Hz, 2 H), | | | |
| | 5.13-5.03 (m, 1 H), 4.56-4.45 (m, 2 H), 4.41- | | | |
| | 4.29 (m, 2 H), 4.25-4.08 (m, 2 H), 3.96-3.88 | | | |
| | (m, 2 H), 3.32-3.19 (m, 5 H), 3.03-2.78 (m, 6 | | | |
| | H), 2.64-2.55 (m, 1 H), 2.45-2.31 (m, 3 H), | | | |
| | 2.15-2.05 (m, 1 H), 2.00-1.84 (m, 5 H), 1.73- | | | |
| | 1.62 (m, 5 H). | | | |
| A184 | 1H NMR (400 MHz, DMSO) δ 10.96 (s, 1H), | 810.4 | 811.4 | |
| | 7.39 (d, J = 7.6 Hz, 1H), 7.26 (d, J = 7.6 Hz, | | | |
| | 1H), 7.12-7.05 (m, 3H), 6.77 (d, J = 6.4 Hz, | | | |
| | 2H), 6.60-6.51 (m, 2H), 6.50-6.44 (m, | | | |
| | 1H), 6.10 (d, J = 13.2 Hz, 1H), 5.66 (d, J = | | | |
| | 8.0 Hz, 1H), 5.12-5.04 (m, 1H), 4.62 (d, J = | | | |
| | 5.2 Hz, 1H), 4.54-4.48 (m, 2H), 4.39-4.32 | | | |
| | (m, 1H), 4.27-4.17 (m, 1H), 3.88-3.80 (m, | | | |
| | 2H), 3.73-3.68 (m, 2H), 3.24-3.20 (m, | | | |
| | 1H), 2.99-2.84 (m, 7H), 2.82-2.74 (m, | | | |
| | 2H), 2.68-2.55 (m, 1H), 2.42-2.32 (m, | | | |
| | 4H), 2.30-2.23 (m, 2H), 2.18-2.10 (m, | | | |
| | 1H), 2.03-1.77 (m, 8H), 1.69-1.57 (m, 3H). | | | |
| A185 | 1H NMR (400 MHz, DMSO) δ 10.92 (s, 1H), | 811.4 | 812.3 | |
| | 7.16 (d, J = 10.4 Hz, 1H), 7.13-7.04 (m, | | | |
| | 3H), 7.00 (d, J = 8.4 Hz, 1H), 6.76 (d, J = 6.8 | | | |
| | Hz, 2H), 6.60-6.52 (m, 2H), 6.49-6.43 (m, | | | |
| | 1H), 6.10 (d, J = 13.2 Hz, 1H), 5.65 (d, J = | | | |
| | 8.4 Hz, 1H), 5.07-4.99 (m, 1H), 4.62 (d, J = | | | |
| | 5.2 Hz, 1H), 4.37-4.31 (m, 1H), 4.26 (d, J = | | | |
| | 16.8 Hz, 1H), 4.10 (d, J = 16.8 Hz, 1H), 3.99- | | | |
| | 3.91 (m, 1H), 3.86-3.77 (m, 3H), 3.73- | | | |
| | 3.65 (m, 2H), 3.24-3.10 (m, 3H), 2.97- | | | |
| | 2.82 (m, 8H), 2.77-2.65 (m, 1H), 2.62- | | | |
| | 2.54 (m, 1H), 2.44-2.32 (m, 4H), 2.31- | | | |
| | 2.24 (m, 2H), 2.21-2.06 (m, 2H), 2.00- | | | |
| | 1.90 (m, 1H), 1.89-1.80 (m, 2H), 1.74 (t, J = | | | |
| | 10.8 Hz, 1H), 1.67-1.57 (m, 1H). | | | |
| A186 | ¹H NMR (400 MHz, MeOD) δ 7.39 (s, 1H), | 750.4 | 751.3 | |
| | 7.11 (dd, J = 10.7, 4.8 Hz, 4H), 6.88-6.77 | | | |
| | (m, 2H), 6.69 (d, J = 8.4 Hz, 1H), 6.66-6.57 | | | |
| | (m, 3H), 6.51 (dd, J = 8.3, 2.6 Hz, 1H), 6.32 | | | |
| | (d, J = 8.6 Hz, 2H), 5.12 (dd, J = 13.3, 5.1 Hz, | | | |
| | 1H), 4.57 (s, 2H), 4.43 (t, J = 13.4 Hz, 2H), | | | |
| | 4.18 (d, J = 5.0 Hz, 1H), 3.56 (d, J = 13.2 Hz, | | | |
| | 3H), 3.38 (s, 2H), 3.00 (dd, J = 11.7, 5.9 Hz, | | | |
| | 2H), 2.90 (dd, J = 15.5, 10.6 Hz, 1H), 2.77 (d, | | | |
| | J = 15.4 Hz, 3H), 2.62 (t, J = 11.6 Hz, 2H), | | | |
| | 2.47 (dt, J = 12.9, 8.3 Hz, 1H), 2.18 (dd, J = | | | |
| | 13.2, 5.6 Hz, 5H), 1.99 (t, J = 17.1 Hz, 3H), | | | |
| | 1.87 (d, J = 10.8 Hz, 3H), 1.76 (d, J = 7.2 Hz, | | | |
| | 1H), 1.40 (d, J = 10.7 Hz, 2H). | | | |
| A187 | 1 H NMR (400 MHz, DMSO-d6) δ 10.96 (s, | 812.4 | 813.5 | |
| | 1 H), 7.39 (d, J = 7.6 Hz, 1 H), 7.27 (d, J = | | | |
| | 7.6 Hz, 1 H), 7.16-7.03 (m, 3 H), 6.76 (d, J = | | | |
| | 6.0 Hz, 2 H), 6.64-6.52 (m, 2 H), 6.51-6.44 | | | |
| | (m, 1 H), 6.25-6.14 (m, 2 H), 5.08 (dd, J = | | | |
| | 13.2, 5.2 Hz, 1 H), 4.67 (d, J = 5.2 Hz, 1 H), | | | |
| | 4.59-4.46 (m, 2 H), 4.43-4.17 (m, 2 H), 3.28- | | | |
| | 3.18 (m, 5 H), 3.00-2.83 (m, 8 H), 2.62-2.54 | | | |
| | (m, 2 H), 2.43-2.31 (m, 3 H), 2.20-2.09 (m, 1 | | | |
| | H), 2.03-1.85 (m, 5 H), 1.76-1.59 (m, 5 H), | | | |
| | 1.47-1.24 (m, 5 H). | | | |
| A188 | 1 H NMR (400 MHz, DMSO-d6) δ 10.93 (s, | 813.4 | 814.5 | |
| | 1 H), 9.15 (s, 1 H), 7.17-7.01 (m, 4 H), 6.93 | | | |
| | (s, 1 H), 6.81-6.69 (m, 2 H), 6.64-6.51 (m, 2 | | | |
| | H), 6.51-6.43 (m, 1 H), 6.25-6.14 (m, 2 H), | | | |
| | 5.02 (dd, J = 13.2, 4.8 Hz, 1 H), 4.67 (d, J = | | | |
| | 5.2 Hz, 1 H), 4.33-4.11 (m, 3 H), 3.95-3.87 | | | |
| | (m, 1 H), 3.81 (d, J = 10.4 Hz, 1 H), 3.26-3.13 | | | |
| | (m, 5 H), 3.00-2.84 (m, 8 H), 2.80-2.72 (m, 1 | | | |
| | H), 2.62-2.54 (m, 2 H), 2.41-2.30 (m, 3 H), | | | |
| | 2.21-2.02 (m, 2 H), 2.00-1.92 (m, 1 H), 1.77- | | | |
| | 1.59 (m, 4 H), 1.50-1.34 (m, 3 H), 1.33-1.21 | | | |
| | (m, 2 H). | | | |
| A189 | 1 H NMR (400 MHz, DMSO-d6) δ 10.96 (s, | 812.4 | 813.4 | |
| | 1 H), 8.29-8.14 (m, 1 H), 7.45 (s, 1 H), 7.13- | | | |
| | 6.98 (m, 4 H), 6.76 (d, J = 6.0 Hz, 2 H), 6.62- | | | |
| | 6.44 (m, 3 H), 6.26-6.14 (m, 2 H), 5.13-5.02 | | | |
| | (m, 1 H), 4.67 (d, J = 5.2 Hz, 1 H), 4.46 (s, 2 | | | |
| | H), 4.36-4.18 (m, 2 H), 3.28-3.19 (m, 5 H), | | | |
| | 2.99-2.84 (m, 8 H), 2.63-2.56 (m, 2 H), 2.41- | | | |
| | 2.31 (m, 3 H), 2.20-2.09 (m, 1 H), 2.03-1.85 | | | |
| | (m, 5 H), 1.77-1.60 (m, 5 H), 1.48-1.36 (m, 3 | | | |
| | H), 1.32-1.21 (m, 2 H). | | | |
| A190 | 1 H NMR (400 MHz, DMSO-d6) δ 10.84 (s, | 827.4 | 828.5 | |
| | 1 H), 8.83 (d, J = 8.4 Hz, 1 H), 8.37-8.17 (m, | | | |
| | 1 H), 7.86 (d, J = 7.6 Hz, 1 H), 7.60 (d, J = | | | |
| | 7.6 Hz, 1 H), 7.12-7.03 (m, 3 H), 6.75 (d, J = | | | |
| | 6.0 Hz, 2 H), 6.64-6.43 (m, 3 H), 6.22-6.11 | | | |
| | (m, 2 H), 4.79-4.64 (m, 2 H), 4.48 (s, 2 H), | | | |
| | 3.27-3.21 (m, 2 H), 2.98-2.72 (m, 13 H), | | | |
| | 2.41-2.36 (m, 2 H), 2.23-2.11 (m, 2 H), 2.04- | | | |
| | 1.82 (m, 7 H), 1.71-1.53 (m, 7 H), 1.46-1.38 | | | |
| | (m, 2 H). | | | |
| A191 | 1 H NMR (400 MHz, DMSO-d6) δ 10.96 (s, | 798.4 | 799.5 | |
| | 1 H), 7.46 (s, 1 H), 7.18-7.03 (m, 3 H), 6.98 | | | |
| | (S, 1 H), 6.77 (d, J = 6.8 Hz, 2 H), 6.63-6.52 | | | |
| | (m, 3 H), 6.51-6.34 (m, 2 H), 5.07 (dd, J = | | | |
| | 13.2, 4.8 Hz, 1 H), 4.60 (d, J = 5.2 Hz, 1 H), | | | |
| | 4.51-4.43 (m, 2 H), 4.37-4.18 (m, 2 H), 3.26- | | | |
| | 3.17 (m, 4 H), 3.00 (s, 3 H), 2.96-2.78 (m, 5 | | | |
| | H), 2.65-2.54 (m, 3 H), 2.40-2.28 (m, 2 H), | | | |
| | 2.20 (d, J = 6.4 Hz, 2 H), 2.04-1.85 (m, 5 H), | | | |
| | 1.81-1.59 (m, 6 H), 1.30-1.17 (m, 2 H). | | | |
| A192 | 1H NMR (400 MHz, DMSO-d6)ppm: δ 10.94 | 795.4 | 796.4 | |
| | (s, 1 H), 8.42 (s, 1 H), 7.13-7.19 (m, 4 H), | | | |
| | 7.10 (d, J = 8.4 Hz, 1 H), 6.83 (d, J = 6.8 Hz, | | | |
| | 2 H), 6.46-6.65 (m, 5 H), 6.20 (d, J = 8.4 Hz, | | | |
| | 2 H), 5.00-5.05 (m, 1 H), 4.34-4.37 (m, 1 H), | | | |
| | 4.24-4.28 (m, 1 H), 4.08-4.15 (m, 2 H), 3.94- | | | |
| | 3.99 (m, 1 H), 3.78-3.85 (m, 1 H), 3.14-3.23 | | | |
| | (m, 4 H), 3.07 (s, 3 H), 2.89-2.99 (m, 5 H), | | | |
| | 2.71-2.82 (m, 3 H), 2.64-2.69 (m, 1 H), 2.28- | | | |
| | 2.41 (m, 4 H), 2.04-2.16 (m, 2 H), 1.91-1.97 | | | |
| | (m, 1 H), 1.66-1.75 (m, 5 H), 1.46-1.75 (m, 2 H). | | | |
| A193 | 1H NMR (400 MHz, DMSO-d6) δ 10.97 (s, 1 | 794.4 | 795.5 | |
| | H), 8.40 (s, 1 H), 7.38 (d, J = 7.6 Hz, 1 H), | | | |
| | 7.26 (d, J = 7.6 Hz, 1 H), 7.11-7.17 (m, 3 H), | | | |
| | 6.82-6.83 (m, 2 H), 6.60-6.65 (m, 2 H), 6.53- | | | |
| | 6.56 (m, 2 H), 6.46-6.49 (m, 1 H), 6.20 (d, J = | | | |
| | 8.8 Hz, 2 H), 5.06-5.10 (m, 1 H), 4.52 (s, 2 | | | |
| | H), 4.38 (d, J = 17.2 Hz, 1 H), 4.21 (d, J = | | | |
| | 17.2 Hz, 1 H), 4.10-4.13 (m, 1 H), 3.17-3.20 | | | |
| | (m, 3 H), 3.07 (m, 3 H), 2.84-2.97 (m, 5 H), | | | |
| | 2.30-2.35 (m, 2 H), 1.86-2.00 (m, 6 H), 1.64- | | | |
| | 1.75 (m, 6 H), 1.44-1.55 (m, 2 H), 1.20-1.29 | | | |
| | (m, 5 H). | | | |
| A194 | ¹H NMR (400 MHz, MeOD) δ 7.84 (s, 1H), | 793.4 | 794.3 | |
| | 7.25 (s, 1H), 7.15-7.06 (m, 4H), 7.03 (s, | | | |
| | 1H), 6.84 (d, J = 6.8 Hz, 2H), 6.67 (dd, J = | | | |
| | 14.5, 5.3 Hz, 2H), 6.52 (dd, J = 8.4, 2.5 Hz, | | | |
| | 1H), 5.06 (dd, J = 13.3, 5.1 Hz, 1H), 4.35- | | | |
| | 4.26 (m, 4H), 4.05-3.91 (m, 2H), 3.51- | | | |
| | 3.42 (m, 2H), 3.40-3.33 (m, 4H), 3.17 (dd, J = | | | |
| | 29.9, 11.1 Hz, 2H), 2.99 (ddd, J = 21.5, | | | |
| | 13.9, 5.7 Hz, 3H), 2.90-2.83 (m, 1H), 2.76 | | | |
| | (dd, J = 16.8, 3.5 Hz, 3H), 2.69-2.53 (m, | | | |
| | 2H), 2.45 (ddd, J = 21.6, 12.7, 7.2 Hz, 2H), | | | |
| | 2.22 (dd, J = 14.7, 9.2 Hz, 1H), 2.11 (dd, J = | | | |
| | 17.3, 8.7 Hz, 3H), 1.84 (d, J = 7.3 Hz, 1H), | | | |
| | 1.71-1.64 (m, 2H), 1.62-1.51 (m, 4H). | | | |
| A195 | ¹H NMR (400 MHz, MeOD) δ 7.85 (s, 1H), | 793.4 | 794.3 | |
| | 7.35 (d, J = 8.3 Hz, 1H), 7.27 (s, 1H), 7.16- | | | |
| | 7.06 (m, 4H), 6.85 (d, J = 6.9 Hz, 2H), 6.68 | | | |
| | (dd, J = 13.1, 5.3 Hz, 2H), 6.52 (dd, J = 8.4, | | | |
| | 2.5 Hz, 1H), 5.08 (dd, J = 13.3, 5.1 Hz, 1H), | | | |
| | 4.46-4.40 (m, 1H), 4.33 (dd, J = 19.1, 7.1 | | | |
| | Hz, 3H), 4.25 (s, 1H), 4.14 (dd, J = 11.0, 7.1 | | | |
| | Hz, 1H), 3.67-3.55 (m, 3H), 3.49-3.43 (m, | | | |
| | 2H), 3.37 (d, J = 5.8 Hz, 3H), 3.19 (d, J = 7.6 | | | |
| | Hz, 2H), 3.01 (ddd, J = 23.1, 17.1, 12.3 Hz, | | | |
| | 3H), 2.90-2.71 (m, 3H), 2.55-2.41 (m, | | | |
| | 2H), 2.22-2.10 (m, 3H), 1.85 (d, J = 7.4 Hz, | | | |
| | 1H), 1.74-1.64 (m, 4H), 1.60-1.51 (m, | | | |
| | 2H), 1.37-1.25 (m, 2H). | | | |
| A196 | 1H NMR (400 MHz, DMSO-d6) δ 10.92 (s, 1 | 839.4 | 840.4 | |
| | H), 9.11 (s, 1 H), 7.07-7.11 (m, 3 H), 7.03 (s, | | | |
| | 1 H), 6.93 (s, 1 H), 6.76 (d, J = 6.8 Hz, 2 H), | | | |
| | 6.54-6.57 (m, 3 H), 6.44-6.47 (m, 1 H), 6.39 | | | |
| | (d, J = 8.8 Hz, 1 H), 4.99-5.04 (m, 1 H), 4.59 | | | |
| | (d, J = 5.2 Hz, 1 H), 4.22-4.29 (m, 2 H), 4.12- | | | |
| | 4.16 (m, 1 H), 3.86-3.91 (m, 1 H), 3.77-3.80 | | | |
| | (m, 1 H), 3.12-3.16 (m, 1 H), 2.99 (s, 3 H), | | | |
| | 2.83-2.89 (m, 5 H), 2.70-2.74 (m, 3 H), 2.55- | | | |
| | 2.60 (m, 1 H), 2.36-2.42 (m, 3 H), 2.27-2.34 | | | |
| | (m, 2 H), 1.93-2.13 (m, 5 H), 1.62-1.74 (m, 5 | | | |
| | H), 1.55-1.57 (m, 2 H), 1.41-1.45 (s, 3 H). | | | |
| A197 | 1H NMR (400 MHz, DMSO-d6) δ 10.92 (s, 1 | 839.4 | 840.4 | |
| | H), 9.13 (s, 1 H), 7.17 (d, J = 8.0 Hz, 1 H), | | | |
| | 7.07-7.11 (m, 3 H), 7.00 (d, J = 8.0 Hz, 1 H), | | | |
| | 6.76 (d, J = 6.4 Hz, 2 H), 6.54-6.57 (m, 3 H), | | | |
| | 6.38-6.47 (m, 2 H), 5.00-5.05 (m, 1 H), 4.59 | | | |
| | (d, J = 4.8 Hz, 1 H), 4.24-4.36 (m, 2 H), 3.93- | | | |
| | 4.12 (m, 2 H), 3.80 (d, J = 11.2 Hz, 1 H), | | | |
| | 3.13-3.21 (m, 2 H), 2.99 (s, 3 H), 2.83-2.92 | | | |
| | (m, 6 H), 2.70-2.78 (m, 3 H), 2.33-2.45 (m, 4 | | | |
| | H), 2.08-2.13 (m, 1 H), 1.88-2.01 (m, 4 H), | | | |
| | 1.62-1.76 (m, 5 H), 1.56 (s, 2 H), 1.41-1.45 | | | |
| | (m, 2 H), 1.24 (s, 1 H). | | | |
| A198 | 1 H NMR (400 MHz, DMSO-d6) δ 10.96 (s, | 838.4 | 839.8 | |
| | 1 H), 7.39 (d, J = 8.0 Hz, 1 H), 7.26 (d, J = | | | |
| | 7.6 Hz, 1 H), 7.17-7.04 (m, 3 H), 6.76 (d, J = | | | |
| | 6.8 Hz, 2 H), 6.60-6.51 (m, 3 H), 6.47-6.35 | | | |
| | (m, 2 H), 5.08 (dd, J = 13.2, 4.8 Hz, 1 H), | | | |
| | 4.59 (d, J = 5.2 Hz, 1 H), 4.55-4.47 (m, 2 H), | | | |
| | 4.41-4.33 (m, 1 H), 4.25-4.16 (m, 1 H), 3.29- | | | |
| | 3.23 (m, 2 H), 3.04-2.69 (m, 14 H), 2.62-2.54 | | | |
| | (m, 1 H), 2.43-2.34 (m, 3 H), 2.32-2.24 (m, 1 | | | |
| | H), 2.02-1.86 (m, 7 H), 1.72-1.61 (m, 5 H), | | | |
| | 1.59-1.52 (m, 2 H), 1.46-1.38 (m, 2 H). | | | |
| A199 | 1 H NMR (400 MHz, DMSO-d6) δ 10.96 (s, | 838.4 | 839.8 | |
| | 1 H), 7.45 (s, 1 H), 7.16-7.05 (m, 3 H), 7.00 | | | |
| | (s, 1 H), 6.76 (d, J = 6.8 Hz, 2 H), 6.60-6.52 | | | |
| | (m, 3 H), 6.48-6.36 (m, 2 H), 5.07 (dd, J = | | | |
| | 13.2, 4.8 Hz, 1 H), 4.59 (d, J = 5.2 Hz, 1 H), | | | |
| | 4.48-4.40 (m, 2 H), 4.36-4.18 (m, 2 H), 3.30- | | | |
| | 3.21 (m, 2 H), 3.01-2.72 (m, 13 H), 2.64-2.56 | | | |
| | (m, 1 H), 2.42-2.34 (m, 3 H), 2.33-2.24 (m, 1 | | | |
| | H), 2.00-1.83 (m, 7 H), 1.71-1.61 (m, 5 H), | | | |
| | 1.59-1.52 (m, 2 H), 1.47-1.38 (m, 2 H). | | | |
| A200 | 1 H NMR (400 MHz, DMSO-d6) δ 10.98 (s, | 872.4 | 873.7 | |
| | 1 H), 8.23 (s, 1 H), 7.42 (s, 1 H), 7.13-7.02 | | | |
| | (m, 3 H), 6.75 (d, J = 6.4 Hz, 2 H), 6.62-6.53 | | | |
| | (m, 2 H), 6.50-6.44 (m, 1 H), 6.25-6.12 (m, 2 | | | |
| | H), 5.11-4.99 (m, 1 H), 4.70-4.63 (m, 1 H), | | | |
| | 4.58-4.45 (m, 2 H), 4.37-4.15 (m, 2 H), 3.27- | | | |
| | 3.22 (m, 1 H), 3.01-2.70 (m, 13 H), 2.64-2.56 | | | |
| | (m, 1 H), 2.43-2.36 (m, 3 H), 2.21-2.10 (m, 1 | | | |
| | H), 2.01-1.87 (m, 7 H), 1.71-1.52 (m, 7 H), | | | |
| | 1.46-1.38 (m, 2 H). | | | |
| A201 | ¹H NMR (400 MHz, MeOD) δ 7.84 (s, 1H), | 792.4 | 793.4 | |
| | 7.37 (dd, J = 17.5, 7.7 Hz, 2H), 7.25 (d, J = | | | |
| | 1.2 Hz, 1H), 7.10 (dt, J = 6.8, 4.6 Hz, 3H), | | | |
| | 6.84 (d, J = 6.7 Hz, 2H), 6.67 (dd, J = 15.4, | | | |
| | 5.4 Hz, 2H), 6.51 (dd, J = 8.3, 2.5 Hz, 1H), | | | |
| | 5.13 (dd, J = 13.3, 5.2 Hz, 1H), 4.59 (s, 2H), | | | |
| | 4.41 (q, J = 17.0 Hz, 2H), 4.29 (d, J = 5.6 Hz, | | | |
| | 1H), 3.50-3.42 (m, 2H), 3.38-3.34 (m, | | | |
| | 3H), 3.11 (d, J = 13.4 Hz, 2H), 3.06-2.96 | | | |
| | (m, 2H), 2.92-2.83 (m, 1H), 2.81-2.72 (m, | | | |
| | 3H), 2.70-2.58 (m, 1H), 2.55-2.38 (m, | | | |
| | 4H), 2.18-2.07 (m, 5H), 1.86 (d, J = 8.4 Hz, | | | |
| | 3H), 1.73-1.66 (m, 2H), 1.62-1.52 (m, 4H). | | | |
| A202 | ¹H NMR (400 MHz, MeOD) δ 7.84 (s, 1H), | 792.4 | 793.3 | |
| | 7.37 (s, 1H), 7.25 (d, J = 1.2 Hz, 1H), 7.13- | | | |
| | 7.05 (m, 4H), 6.85 (d, J = 6.7 Hz, 2H), 6.67 | | | |
| | (dd, J = 15.2, 5.4 Hz, 2H), 6.51 (dd, J = 8.3, | | | |
| | 2.6 Hz, 1H), 5.11 (dd, J = 13.3, 5.1 Hz, 1H), | | | |
| | 4.55 (d, J = 16.4 Hz, 2H), 4.42 (t, J = 12.1 | | | |
| | Hz, 2H), 4.29 (d, J = 5.8 Hz, 1H), 3.45 (s, | | | |
| | 2H), 3.37-3.34 (m, 3H), 3.13 (s, 2H), 3.06- | | | |
| | 3.01 (m, 1H), 2.79 (s, 3H), 2.65 (d, J = 7.1 | | | |
| | Hz, 1H), 2.55-2.42 (m, 4H), 2.09 (dd, J = | | | |
| | 25.6, 14.7 Hz, 6H), 1.88 (d, J = 13.5 Hz, 3H), | | | |
| | 1.69 (s, 2H), 1.58 (dd, J = 22.7, 7.5 Hz, 5H). | | | |
| A203 | 1 H NMR (400 MHz, DMSO-d6) δ 10.95 (s, | 852.4 | 853.8 | |
| | 1 H), 7.17-7.03 (m, 4 H), 6.75 (d, J = 6.4 Hz, | | | |
| | 2 H), 6.61-6.44 (m, 3 H), 6.23-6.13 (m, 2 H), | | | |
| | 5.03 (dd, J = 13.2, 5.2 Hz, 1 H), 4.67 (d, J = | | | |
| | 5.2 Hz, 1 H), 4.49-4.41 (m, 2 H), 4.31-4.11 | | | |
| | (m, 2 H), 3.27-3.21 (m, 1 H), 3.03-2.66 (m, | | | |
| | 13 H), 2.62-2.52 (m, 4 H), 2.48-2.32 (m, 4 H), | | | |
| | 2.19-2.10 (m, 1 H), 2.04-1.82 (m, 7 H), 1.70- | | | |
| | 1.52 (m, 7 H), 1.47-1.39 (m, 2 H). | | | |
| A204 | ¹H NMR (400 MHz, MeOD) δ 8.51 (s, 2H), | 785.4 | 786.3 | |
| | 7.37 (d, J = 8.3 Hz, 1H), 7.12 (d, J = 8.4 Hz, | | | |
| | 1H), 7.09-6.95 (m, 3H), 6.79 (d, J = 7.8 Hz, | | | |
| | 2H), 6.60 (dd, J = 10.0, 5.9 Hz, 2H), 6.50 (d, | | | |
| | J = 8.2 Hz, 1H), 6.21 (d, J = 12.8 Hz, 1H), | | | |
| | 6.02 (d, J = 7.5 Hz, 1H), 5.09 (dd, J = 13.4, | | | |
| | 5.1 Hz, 1H), 4.73 (d, J = 5.1 Hz, 1H), 4.42 (d, | | | |
| | J = 9.1 Hz, 1H), 4.34 (t, J = 13.8 Hz, 2H), | | | |
| | 4.24-4.10 (m, 2H), 3.68 (d, J = 32.3 Hz, | | | |
| | 5H), 3.48-3.37 (m, 2H), 3.23 (s, 2H), 2.98 | | | |
| | (dd, J = 17.8, 10.6 Hz, 5H), 2.91-2.69 (m, | | | |
| | 2H), 2.54-2.36 (m, 2H), 2.34-2.15 (m, | | | |
| | 2H), 2.08 (d, J = 32.4 Hz, 2H), 1.69 (s, 1H), | | | |
| | 1.31 (d, J = 17.8 Hz, 4H). | | | |
| A205 | ¹H NMR (400 MHz, MeOD) δ 8.53 (s, 2H), | 784.4 | 785.3 | |
| | 7.60-7.43 (m, 1H), 7.16 (d, J = 3.8 Hz, 1H), | | | |
| | 7.07 (d, J = 7.5 Hz, 3H), 6.79 (d, J = 6.6 Hz, | | | |
| | 2H), 6.61 (d, J = 11.1 Hz, 2H), 6.53-6.37 | | | |
| | (m, 1H), 6.26-6.15 (m, 1H), 6.10-5.97 (m, | | | |
| | 1H), 5.34 (t, J = 4.6 Hz, 1H), 5.13 (dd, J = | | | |
| | 13.7, 5.1 Hz, 1H), 4.71 (d, J = 14.9 Hz, 1H), | | | |
| | 4.62 (s, 2H), 4.50-4.36 (m, 2H), 3.70 (s, | | | |
| | 2H), 3.62 (d, J = 13.5 Hz, 2H), 3.48 (s, 2H), | | | |
| | 3.07-3.01 (m, 3H), 2.98-2.92 (m, 2H), | | | |
| | 2.81 (t, J = 22.2 Hz, 2H), 2.39 (s, 3H), 2.22- | | | |
| | 2.17 (m, 2H), 2.03 (s, 4H), 1.64 (d, J = 26.2 | | | |
| | Hz, 3H), 1.34 (s, 3H). | | | |
| A206 | ¹H NMR (400 MHz, MeOD) δ 8.54 (s, 1H), | 784.4 | 785.3 | |
| | 7.57-7.41 (m, 2H), 7.12-7.00 (m, 3H), | | | |
| | 6.79 (d, J = 7.2 Hz, 2H), 6.62 (dd, J = 7.6, 5.4 | | | |
| | Hz, 2H), 6.47 (ddd, J = 17.5, 6.5, 2.4 Hz, 1H), | | | |
| | 6.26-6.17 (m, 1H), 6.04 (dt, J = 14.0, 6.9 | | | |
| | Hz, 1H), 5.19-5.09 (m, 1H), 4.77-4.65 (m, | | | |
| | 3H), 4.43 (q, J = 17.1 Hz, 2H), 4.29-3.98 | | | |
| | (m, 1H), 3.67 (dd, J = 38.2, 16.0 Hz, 5H), | | | |
| | 3.47 (dd, J = 27.4, 13.8 Hz, 2H), 3.28-3.18 | | | |
| | (m, 2H), 3.08-2.84 (m, 7H), 2.82-2.73 (m, | | | |
| | 1H), 2.56-2.33 (m, 3H), 2.32-2.10 (m, | | | |
| | 3H), 2.10-1.92 (m, 3H), 1.68 (d, J = 11.3 | | | |
| | Hz, 1H), 1.36-1.22 (m, 1H). | | | |
| A212 | 1H NMR (400 MHz, DMSO-d6) δ 10.98 (s, 1 | 846.4 | 847.4 | |
| | H), 9.13 (s, 1 H), 8.15 (s, 1 H), 7.50-7.35 (m, | | | |
| | 1 H), 7.15-7.03 (m, 3 H), 6.80-6.71 (m, 2 H), | | | |
| | 6.61-6.53 (m, 2 H), 6.51-6.43 (m, 1 H), 6.24- | | | |
| | 6.14 (m, 2 H), 5.12-5.00 (m, 1 H), 4.67 (d, J = | | | |
| | 5.2 Hz, 1 H), 4.62-4.50 (m, 2 H), 4.37-4.14 | | | |
| | (m, 2 H), 3.29-3.14 (m, 5 H), 3.01-2.83 (m, 8 | | | |
| | H), 2.64-2.55 (m, 2 H), 2.45-2.30 (m, 3 H), | | | |
| | 2.22-1.89 (m, 6 H), 1.79-1.58 (m, 5 H), 1.50- | | | |
| | 1.35 (m, 3 H), 1.33-1.21 (m, 2 H). | | | |
| A213 | 1 H NMR (400 MHz, DMSO-d6) δ 10.96 (s, | 826.4 | 827.8 | |
| | 1 H), 9.14 (s, 1 H), 7.15-7.04 (m, 4 H), 6.82- | | | |
| | 6.71 (m, 2 H), 6.62-6.53 (m, 2 H), 6.52-6.46 | | | |
| | (m, 1 H), 6.25-6.15 (m, 2 H), 5.04 (dd, J = | | | |
| | 13.2, 5.2 Hz, 1 H), 4.67 (d, J = 4.8 Hz, 1 H), | | | |
| | 4.53-4.44 (m, 2 H), 4.34-4.10 (m, 2 H), 3.28- | | | |
| | 3.20 (m, 4 H), 3.03-2.83 (m, 9 H), 2.63-2.53 | | | |
| | (m, 6 H), 2.43-2.34 (m, 1 H), 2.21-2.07 (m, 3 | | | |
| | H), 1.99-1.86 (m, 3 H), 1.77-1.60 (m, 5 H), | | | |
| | 1.51-1.36 (m, 3 H), 1.33-1.21 (m, 2 H). | | | |
| A214 | 1H NMR (400 MHz, DMSO-d6) δ 10.94 (s, 1 | 804.4 | 805.8 | |
| | H), 8.16 (s, 1 H), 7.17-7.10 (m, 4 H), 6.83 (d, | | | |
| | J = 7.2 Hz, 2 H), 6.65-6.59 (m, 2 H), 6.54- | | | |
| | 6.46 (m, 3 H), 6.19 (d, J = 8.4 Hz, 2 H), 5.03 | | | |
| | (dd, J = 13.2, 5.2 Hz, 1 H), 4.49-4.41 (m, 2 | | | |
| | H), 4.31-4.10 (m, 3 H), 2.98-2.91 (m, 4 H), | | | |
| | 2.89-2.78 (m, 6 H), 2.61-2.53 (m, 5 H), 2.44- | | | |
| | 2.37 (m, 3 H), 2.01-1.84 (m, 7 H), 1.71-1.58 | | | |
| | (m, 5 H), 1.51-1.38 (m, 4 H), 1.10 (s, 1 H). | | | |
| A216 | 1 H NMR (400 MHz, DMSO-d6) δ 10.95 (s, | 852.4 | 853.8 | |
| | 1 H), 8.27 (s, 1 H), 7.67-7.48 (m, 1 H), 7.36- | | | |
| | 7.23 (m, 1 H), 7.15-7.05 (m, 5 H), 6.78 (d, J = | | | |
| | 6.8 Hz, 3 H), 6.62-6.40 (m, 4 H), 6.10 (d, J = | | | |
| | 13.6 Hz, 1 H), 5.68 (d, J = 8.4 Hz, 1 H), 5.09- | | | |
| | 4.96 (m, 1 H), 4.64-4.59 (m, 1 H), 4.50-4.42 | | | |
| | (m, 2 H), 4.33-4.25 (m, 1 H), 4.19-4.11 (m, 1 | | | |
| | H), 3.55-3.48 (m, 5 H), 3.24-3.20 (m, 2 H), | | | |
| | 2.93-2.92 (m, 3 H), 2.88-2.84 (m, 1 H), 2.83- | | | |
| | 2.75 (m, 3 H), 2.55-2.53 (m, 3 H), 2.12-2.08 | | | |
| | (m, 2 H), 1.93-1.85 (m, 6 H), 1.71-1.62 (m, 5 | | | |
| | H), 1.46-1.40 (m, 2 H), 0.92-0.87 (m, 1 H). | | | |
| A217 | 1 H NMR (400 MHz, DMSO-d6) δ 10.97 (s, | 846.4 | 847.3 | |
| | 1 H), 8.23 (s, 1 H), 7.42 (s, 1 H), 7.13-7.04 | | | |
| | (m, 3 H), 6.79-6.71 (m, 2 H), 6.63-6.53 (m, 2 | | | |
| | H), 6.51-6.42 (m, 1 H), 6.25-6.14 (m, 2 H), | | | |
| | 5.04 (dd, J = 13.2, 5.2 Hz, 1 H), 4.67 (d, J = | | | |
| | 5.2 Hz, 1 H), 4.62-4.53 (m, 2 H), 4.36-4.28 | | | |
| | (m, 1 H), 4.22-4.15 (m, 1 H), 3.28-3.20 (m, 3 | | | |
| | H), 3.05-2.80 (m, 9 H), 2.63-2.55 (m, 2 H), | | | |
| | 2.42-2.31 (m, 3 H), 2.20-2.10 (m, 1 H), 2.02- | | | |
| | 1.85 (m, 5 H), 1.78-1.60 (m, 5 H), 1.47-1.24 | | | |
| | (m, 5 H). | | | |
| A218 | 1 H NMR (400 MHz, DMSO-d6) δ 10.97 (s, | 872.4 | 873.8 | |
| | 1 H), 8.21 (s, 1 H), 7.41 (s, 1 H), 7.11-7.03 | | | |
| | (m, 3 H), 6.75 (d, J = 6.0 Hz, 2 H), 6.61-6.52 | | | |
| | (m, 2 H), 6.50-6.44 (m, 1 H), 6.23-6.13 (m, 2 | | | |
| | H), 5.09-4.99 (m, 1 H), 4.67 (d, J = 5.2 Hz, 1 | | | |
| | H), 4.59-4.50 (m, 2 H), 4.35-4.29 (m, 1 H), | | | |
| | 4.22-4.16 (m, 1 H), 3.28-3.21 (m, 1 H), 3.04- | | | |
| | 2.66 (m, 13 H), 2.63-2.55 (m, 1 H), 2.47-2.29 | | | |
| | (m, 4 H), 2.19-2.09 (m, 1 H), 2.05-1.84 (m, 7 | | | |
| | H), 1.74-1.63 (m, 4 H), 1.59-1.52 (m, 2 H), | | | |
| | 1.48-1.38 (m, 2 H). | | | |
| A219 | 1 H NMR (400 MHz, DMSO-d6) δ 10.95 (s, | 852.4 | 853.8 | |
| | 1 H), 7.23 (s, 1 H), 7.13-7.00 (m, 3 H), 6.75 | | | |
| | (d, J = 6.4 Hz, 2 H), 6.62-6.51 (m, 2 H), 6.50- | | | |
| | 6.40 (m, 1 H), 6.25-6.10 (m, 2 H), 5.02 (dd, J = | | | |
| | 13.2, 5.2 Hz, 1 H), 4.67 (d, J = 4.8 Hz, 1 H), | | | |
| | 4.43 (s, 2 H), 4.29-4.10 (m, 2 H), 3.27-3.21 | | | |
| | (m, 2 H), 3.00-2.71 (m, 13 H), 2.64-2.56 (m, | | | |
| | 1 H), 2.45-2.34 (m, 6 H), 2.19-2.10 (m, 1 H), | | | |
| | 2.03-1.82 (m, 7 H), 1.70-1.54 (m, 7 H), 1.46- | | | |
| | 1.38 (m, 2 H). | | | |
| A220 | 1 H NMR (400 MHz, DMSO-d6) δ 10.95 (s, | 852.4 | 853.8 | |
| | 1 H), 8.23 (s, 1 H), 7.23 (s, 1 H), 7.16-7.01 | | | |
| | (m, 3 H), 6.75 (d, J = 6.4 Hz, 2 H), 6.61-6.52 | | | |
| | (m, 2 H), 6.50-6.45 (m, 1 H), 6.24-6.11 (m, 2 | | | |
| | H), 5.02 (dd, J = 13.2, 5.2 Hz, 1 H), 4.66 (d, J = | | | |
| | 5.2 Hz, 1 H), 4.47-4.39 (m, 2 H), 4.29-4.12 | | | |
| | (m, 2 H), 3.27-3.22 (m, 1 H), 3.00-2.71 (m, | | | |
| | 13 H), 2.61-2.55 (m, 1 H), 2.44-2.33 (m, 6 H), | | | |
| | 2.21-2.11 (m, 1 H), 2.03-1.82 (m, 7 H), 1.69- | | | |
| | 1.52 (m, 7 H), 1.47-1.38 (m, 2 H). | | | |
| A221 | 1H NMR (400 MHz, DMSO-d6) δ 10.97 (s, | 873.3 | 874.2 | |
| | 1H), 8.24 (s, 1H), 7.29 (s, 1H), 7.11-7.02 | | | |
| | (m, 3H), 6.78-6.71 (m, 2H), 6.59-6.53 (m, | | | |
| | 2H), 6.50-6.43 (m, 1H), 6.24-6.13 (m, | | | |
| | 2H), 5.11-5.01 (m, 1H), 4.66 (d, J = 5.6 Hz, | | | |
| | 1H), 4.43 (t, J = 10.4 Hz, 1H), 4.36-4.24 (m, | | | |
| | 2H), 4.18 (d, J = 17.6 Hz, 1H), 3.45-3.43 | | | |
| | (m, 1H), 3.26-3.16 (m, 3H), 3.00-2.92 (m, | | | |
| | 5H), 2.90-2.81 (m, 5H), 2.80-2.69 (m, | | | |
| | 3H), 2.61-2.53 (m, 1H), 2.47-2.42 (m, | | | |
| | 2H), 2.39-2.28 (m, 3H), 2.19-2.11 (m, | | | |
| | 1H), 2.01-1.91 (m, 3H), 1.72-1.54 (m, | | | |
| | 5H), 1.45-1.40 (m, 2H). | | | |
| A222 | 1 H NMR (400 MHz, DMSO-d6) δ 10.93 (s, | 868.4 | 869.8 | |
| | 1 H), 8.18 (s, 1 H), 7.12-7.00 (m, 4 H), 6.79- | | | |
| | 6.71 (m, 2 H), 6.61-6.54 (m, 2 H), 6.49-6.45 | | | |
| | (m, 1 H), 6.24-6.14 (m, 2 H), 4.99 (dd, J = | | | |
| | 13.2, 5.2 Hz, 1 H), 4.67 (d, J = 5.2 Hz, 1 H), | | | |
| | 4.49-4.40 (m, 2 H), 4.29-4.21 (m, 1 H), 4.16- | | | |
| | 4.05 (m, 1 H), 3.83-3.79 (m, 3 H), 2.97-2.71 | | | |
| | (m, 13 H), 2.43-2.31 (m, 4 H), 2.19-2.09 (m, | | | |
| | 1 H), 1.99-1.89 (m, 7 H), 1.72-1.51 (m, 8 H), | | | |
| | 1.49-1.36 (m, 3 H). | | | |
| A223 | 1 H NMR (400 MHz, DMSO-d6) δ 10.93 (s, | 868.4 | 869.8 | |
| | 1 H), 8.24 (s, 1 H), 7.13-7.00 (m, 4 H), 6.82- | | | |
| | 6.70 (m, 2 H), 6.61-6.45 (m, 3 H), 6.25-6.11 | | | |
| | (m, 2 H), 4.99 (dd, J = 13.2, 5.2 Hz, 1 H), | | | |
| | 4.67 (d, J = 5.2 Hz, 1 H), 4.49-4.41 (m, 2 H), | | | |
| | 4.29-4.23 (m, 1 H), 4.12-4.06 (m, 1 H), 3.82 | | | |
| | (s, 3 H), 3.27-3.21 (m, 1 H), 2.98-2.75 (m, 13 | | | |
| | H), 2.44-2.35 (m, 3 H), 2.19-2.09 (m, 1 H), | | | |
| | 2.01-1.91 (m, 7 H), 1.72-1.52 (m, 8 H), 1.49- | | | |
| | 1.37 (m, 2 H). | | | |
| A224 | 1H NMR (400 MHz, DMSO) δ 10.97 (s, 1H), | 847.4 | 848.3 | |
| | 8.29-8.28 (m, 2H), 7.30 (s, 1H), 7.11-7.06 | | | |
| | (m, 3H), 6.77-6.75 (m, 2H), 6.60-6.55 (m, | | | |
| | 2H), 6.49-6.47 (m, 1H), 6.21-6.17 (m, | | | |
| | 2H), 5.08-5.03 (m, 1H), 4.68-4.66 (m, | | | |
| | 1H), 4.48-4.42 (m, 1H), 4.36-4.29 (m, | | | |
| | 2H), 4.21-4.20 (m, 1H), 3.69-3.64 (m, | | | |
| | 2H), 3.02-2.98 (m, 2H), 2.95 (s, 3H), 2.91- | | | |
| | 2.81 (m, 3H), 2.67-2.60 (m, 3H), 2.46- | | | |
| | 2.40 (m, 2H), 2.36-2.24 (m, 3H), 2.18- | | | |
| | 2.13 (m, 1H), 1.75-1.70 (m, 2H), 1.63- | | | |
| | 1.62 (m, 1H), 1.45-1.37 (m, 3H), 1.32- | | | |
| | 1.24 (m, 3H). | | | |
| A225 | 1H NMR (400 MHz, DMSO-d6) δ 10.92 (s, 1 | 842.4 | 843.3 | |
| | H), 9.13 (s, 1 H), 8.15-7.07 (m, 3 H), 7.02 (s, | | | |
| | 1 H), 6.77-6.75 (d, J = 8 Hz, 2 H), 6.59-6.55 | | | |
| | (m, 2 H), 6.49-6.46 (m, 1 H), 6.21-6.18 (m, 2 | | | |
| | H), 5.02-4.97 (dd, J = 4 Hz, J = 12 Hz, 1 H), | | | |
| | 4.68-4.67 (d, J = 4 Hz 1 H), 4.49-4.44 (m, 2 | | | |
| | H), 4.29-4.08 (m, 2H), 3.82 (s, 3 H), 3.24 (s, 5 | | | |
| | H), 2.96 (s, 3 H), 2.90-2.85 (m, 4 H), 2.62- | | | |
| | 2.55 (m, 2 H), 2.42-2.32 (m, 3 H), 2.18-2.13 | | | |
| | (m, 1 H), 2.01-1.92 (m, 5 H), 1.75-1.62 (m, 5 | | | |
| | H), 1.43-1.40 (m, 3 H), 1.27-1.24 (m, 2 H). | | | |
| A226 | 1H NMR (400 MHz, MeOD-d4) δ 7.05-7.08 | 842.4 | 843.5 | |
| | (m, 3 H), 6.94 (s, 1 H), 6.75-6.77 (d, J = 7.6 | | | |
| | Hz, 2 H), 6.60-6.64 (m, 2 H), 6.50-6.53 (d, J = | | | |
| | 10.4 Hz, 1 H), 6.19-6.29 (m, 3 H), 5.05- | | | |
| | 5.10 (dd, J = 13.2 Hz, 5.2 Hz, 1 H), 4.76-4.78 | | | |
| | (d, J = 8.8 Hz, 2 H), 4.57 (s, 2 H), 4.28-4.39 | | | |
| | (m, 2 H), 3.91 (s, 3 H), 3.24-3.35 (m, 3 H), | | | |
| | 3.01 (s, 5 H), 2.85-2.96 (m, 4 H), 2.58-2.83 | | | |
| | (m, 5 H), 2.11-2.20 (m, 5 H), 1.91-1.95 (d, J = | | | |
| | 13.2 Hz, 2 H), 1.79-1.84 (m, 2 H), 1.67-1.70 | | | |
| | (m, 3 H), 1.46 (s, 3 H). | | | |
| A227 | 1H NMR (400 MHz, DMSO-d6) δ 10.96 (s, 1 | 882.4 | 883.1 | |
| | H), 9.13 (s, 1 H), 7.06-7.16 (m, 4 H), 6.73- | | | |
| | 6.79 (m, 2 H), 6.51-6.61 (m, 2 H), 6.47 (dd, J = | | | |
| | 8.4 Hz, 2.0 Hz, 1 H), 6.19 (dd, J = 10.0 Hz, | | | |
| | 2.0 Hz, 2 H), 5.00-5.08 (m, 1 H), 4.65-4.69 | | | |
| | (m, 1 H), 4.43-4.51 (m, 2 H), 4.29 (d, J = 16.8 | | | |
| | Hz, 1 H), 4.14 (d, J = 16.8 Hz, 1 H), 3.65-3.72 | | | |
| | (m, 1 H), 3.20-3.28 (m, 3 H), 2.74-3.03 (m, | | | |
| | 13 H), 2.59-2.63 (m, 1 H), 2.27-2.45 (m, 2 H), | | | |
| | 2.05-2.22 (m, 4 H), 1.83-2.03 (m, 5 H), 1.53- | | | |
| | 1.81 (m, 8 H), 1.43-1.52 (m, 1 H), 1.29-1.41 | | | |
| | (m, 2 H). | | | |
| A228 | 1H NMR (400 MHz, DMSO) δ 10.94 (s, 1H), | 853.4 | 854.4 | |
| | 9.27-8.99 (m, 1H), 7.16-7.02 (m, 4H), | | | |
| | 6.82-6.71 (m, 2H), 6.62-6.53 (m, 2H), | | | |
| | 6.49-6.44 (m, 1H), 6.22-6.14 (m, 2H), | | | |
| | 5.08-4.99 (m, 1H), 4.66 (d, J = 5.2 Hz, 1H), | | | |
| | 4.44 (t, J = 10.4 Hz, 1H), 4.31-4.25 (m, 1H), | | | |
| | 4.21-4.08 (m, 2H), 3.28-3.20 (m, 2H), | | | |
| | 3.16-3.06 (m, 2H), 3.01-2.94 (m, 4H), | | | |
| | 2.92-2.81 (m, 5H), 2.79-2.68 (m, 4H), | | | |
| | 2.62-2.55 (m, 1H), 2.47-2.36 (m, 4H), | | | |
| | 2.33-2.24 (m, 4H), 2.18-2.10 (m, 1H), | | | |
| | 2.00-1.89 (m, 3H), 1.72-1.53 (m, 5H), | | | |
| | 1.48-1.33 (m, 2H). | | | |
| A229 | 1H NMR (400 MHz, DMSO) δ 10.95 (s, 1H), | 827.4 | 828.3 | |
| | 9.13 (s, 1H), 7.12-7.04 (m, 4H), 6.81-6.71 | | | |
| | (m, 2H), 6.60-6.54 (m, 2H), 6.50-6.44 (m, | | | |
| | 1H), 6.25-6.11 (m, 2H), 5.11-5.00 (m, | | | |
| | 1H), 4.67 (d, J = 5.2 Hz, 1H), 4.46 (t, J = 10.4 | | | |
| | Hz, 1H), 4.32-4.25 (m, 1H), 4.24-4.18 (m, | | | |
| | 1H), 4.16-4.08 (m, 1H), 3.29-3.07 (m, | | | |
| | 6H), 3.03-2.94 (m, 5H), 2.91-2.70 (m, | | | |
| | 4H), 2.62-2.54 (m, 2H), 2.47-2.35 (m, | | | |
| | 3H), 2.33-2.13 (m, 6H), 1.99-1.93 (m, | | | |
| | 1H), 1.77-1.57 (m, 3H), 1.46-1.21 (m, 5H). | | | |
| A230 | 1H NMR (400 MHz, DMSO-d6) δ 10.95 (s, | 882.4 | 883.3 | |
| | 1H), 8.30 (s, 1H), 7.14-7.07 (m, 4H), 6.75 | | | |
| | (d, J = 7.60 Hz, 2H), 6.59-6.55 (m, 2H), 6.46 | | | |
| | (d, J = 10.40 Hz, 1H), 6.21-6.18 (m, 2H), | | | |
| | 5.04-5.01 (m, 1H), 4.68-4.67 (m, 1H), | | | |
| | 4.49-4.44 (m, 2H), 4.29 (d, J = 17.20 Hz, | | | |
| | 1H), 4.14 (d, J = 16.80 Hz, 1H), 3.70-3.67 | | | |
| | (m, 1H), 3.28-3.23 (m, 3H), 2.99 (s, 5H), | | | |
| | 2.91-2.84 (m, 3H), 2.77-2.74 (m, 2H), | | | |
| | 2.67-2.60 (m, 1H), 2.54 (s, 3H), 2.45-2.33 | | | |
| | (m, 2H), 2.19-2.11 (m, 4H), 1.96-1.88 (m, | | | |
| | 5H), 1.81-1.76 (m, 1H), 1.67-1.60 (m, | | | |
| | 7H), 1.50-1.43 (m, 1H), 1.36-1.34 (m, 2H). | | | |
| A231 | 1 H NMR (400 MHz, DMSO-d6) δ 10.98 (s, | 854.4 | 855.2 | |
| | 1 H), 9.10 (s, 1 H), 7.43 (s, 1 H), 7.20-7.06 | | | |
| | (m, 3 H), 6.83 (d, J = 6.4 Hz, 2 H), 6.67-6.58 | | | |
| | (m, 2 H), 6.57-6.43 (m, 3 H), 6.20 (d, J = 8.4 | | | |
| | Hz, 2 H), 5.05 (dd, J = 13.2, 5.2 Hz, 1 H), | | | |
| | 4.59-4.48 (m, 2 H), 4.37-4.28 (m, 1 H), 4.23- | | | |
| | 4.10 (m, 2 H), 3.70-3.63 (m, 1 H), 3.17-3.10 | | | |
| | (m, 2 H), 3.03-2.74 (m, 8 H), 2.63-2.55 (m, 2 | | | |
| | H), 2.43-2.34 (m, 1 H), 2.22-2.08 (m, 3 H), | | | |
| | 2.04-1.86 (m, 6 H), 1.76-1.52 (m, 8 H), 1.44- | | | |
| | 1.28 (m, 3 H). | | | |
| A232 | 1 H NMR (400 MHz, DMSO-d6) δ 10.98 (s, | 902.4 | 903.2 | |
| | 1 H), 8.19 (s, 1 H), 7.46-7.37 (m, 1 H), 7.14- | | | |
| | 7.03 (m, 3 H), 6.80-6.62 (m, 2 H), 6.62-6.52 | | | |
| | (m, 2 H), 6.51-6.45 (m, 1 H), 6.27-6.15 (m, 2 | | | |
| | H), 5.05 (dd, J = 13.2, 5.2 Hz, 1 H), 4.67 (d, J = | | | |
| | 5.2 Hz, 1 H), 4.58-4.49 (m, 2 H), 4.40-4.30 | | | |
| | (m, 1 H), 4.25-4.15 (m, 1 H), 3.71-3.66 (m, 1 | | | |
| | H), 3.28-3.23 (m, 2 H), 3.02-2.83 (m, 11 H), | | | |
| | 2.81-2.72 (m, 2 H), 2.64-2.57 (m, 1 H), 2.45- | | | |
| | 2.36 (m, 1 H), 2.22-2.10 (m, 3 H), 2.01-1.87 | | | |
| | (m, 5 H), 1.77-1.55 (m, 8 H), 1.50-1.29 (m, 3 H). | | | |
| A233 | 1H NMR (400 MHz, DMSO-d6) δ 10.98 (s, 1 | 854.4 | 855.4 | |
| | H), 9.11 (s, 1 H), 7.43 (s, 1 H), 7.16-7.11 (m, | | | |
| | 3 H), 6.83-6.82 (d, J = 4 Hz, 2 H), 6.66-6.60 | | | |
| | (m, 2 H), 6.54-6.47 (m, 3 H), 6.21-6.19 (d, 2 | | | |
| | H), 5.07-5.03 (dd, J = 4 Hz, 12 Hz, 1 H), 4.54 | | | |
| | (s, 2 H), 4.36-4.16 (m, 2 H), 4.13-4.12 (m, 1 | | | |
| | H), 3.68-3.65 (m, 2 H), 3.17 (s, 6 H), 3.01- | | | |
| | 2.75 (m, 6 H), 2.19-2.13 (m, 2 H), 2.07-1.90 | | | |
| | (m, 6 H), 1.72-1.62 (m, 5 H), 1.59-1.52 (m, 3 | | | |
| | H), 1.34-1.24 (m, 4 H). | | | |
| A234 | 1H NMR (400 MHz, DMSO-d6) δ 10.96 (s, 1 | 902.4 | 903.3 | |
| | H), 9.11 (s, 1 H), 7.42 (s, 1 H), 7.11-7.05 (m, | | | |
| | 3 H), 6.77 (d, J = 8.0 Hz, 2 H), 6.59-6.55 (m, | | | |
| | 2 H), 6.49-6.46 (m, 1 H), 6.21-6.18 (m, 2 H), | | | |
| | 5.07-5.03 (m, 1 H), 4.68 (, 1 H), 4.57-4.52 | | | |
| | (m, 2 H), 4.36-4.15 (m, 2 H), 3.69-3.67 (m, 1 | | | |
| | H), 3.96 (s, 3 H), 2.83-2.75 (m, 8 H), 2.67- | | | |
| | 2.56 (m, 1 H), 2.36-2.23 (m, 3 H), 2.18-2.13 | | | |
| | (m, 5 H), 1.94-1.89 (m, 5 H), 1.76-1.56 (m, 9 | | | |
| | H), 1.39-1.30 (m, 2 H). | | | |
| A235 | 1 H NMR (400 MHz, DMSO-d6) δ 10.92 (s, | 850.4 | 851.7 | |
| | 1 H), 8.31 (s, 1 H), 7.19-7.08 (m, 3 H), 7.01 | | | |
| | (s, 1 H), 6.83 (d, J = 6.8 Hz, 2 H), 6.68-6.59 | | | |
| | (m, 2 H), 6.56-6.43 (m, 3 H), 6.20 (d, J = 8.4 | | | |
| | Hz, 2 H), 4.99 (dd, J = 13.2, 4.8 Hz, 1 H), | | | |
| | 4.52-4.40 (m, 2 H), 4.31-4.20 (m, 1 H), 4.15- | | | |
| | 4.06 (m, 2 H), 3.82 (s, 3 H), 3.69-3.65 (m, 1 | | | |
| | H), 3.31-3.22 (m, 3 H), 3.16-3.10 (m, 2 H), | | | |
| | 2.97-2.83 (m, 5 H), 2.81-2.71 (m, 2 H), 2.64- | | | |
| | 2.55 (m, 1 H), 2.40-2.31 (m, 1 H), 2.20-2.12 | | | |
| | (m, 2 H), 2.04-1.87 (m, 6 H), 1.75-1.50 (m, 8 | | | |
| | H), 1.45-1.37 (m, 1 H), 1.37-1.24 (m, 2 H). | | | |
| A236 | 1 H NMR (400 MHz, DMSO-d6) δ 10.92 (s, | 850.4 | 851.7 | |
| | 1 H), 8.25 (s, 1 H), 7.18-7.08 (m, 3 H), 7.01 | | | |
| | (s, 1 H), 6.83 (d, J = 6.8 Hz, 2 H), 6.73-6.58 | | | |
| | (m, 2 H), 6.56-6.43 (m, 3 H), 6.20 (d, J = 8.4 | | | |
| | Hz, 2 H), 4.99 (dd, J = 13.2, 5.2 Hz, 1 H), | | | |
| | 4.50-4.38 (m, 2 H), 4.32-4.23 (m, 1 H), 4.14- | | | |
| | 4.07 (m, 2 H), 3.82 (s, 3 H), 3.66-3.65 (m, 1 | | | |
| | H), 3.30-3.21 (m, 3 H), 3.17-3.11 (m, 2 H), | | | |
| | 2.98-2.83 (m, 5 H), 2.80-2.73 (m, 2 H), 2.62- | | | |
| | 2.54 (m, 1 H), 2.39-2.31 (m, 1 H), 2.20-2.13 | | | |
| | (m, 2 H), 2.05-1.87 (m, 6 H), 1.77-1.51 (m, 8 | | | |
| | H), 1.45-1.38 (m, 1 H), 1.36-1.26 (m, 2 H). | | | |

### For “B” Compounds

Compound B142:3-((S)-3-(2-(7-(4-((1R,2S)-6-hydroxy-2-phenyl-1,2,3,4-tetrahydronaphthalen-1-yl)phenyl)-2,7-diazaspiro[3.5]nonan-2-yl)acetyl)-8-oxo-1,2,3,4,4a,5,8,10-octahydro-9H-pyrazino[1′,2′: 4,5][1,4]oxazino[2,3-f]isoindol-9-yl)piperidine-2,6-dione

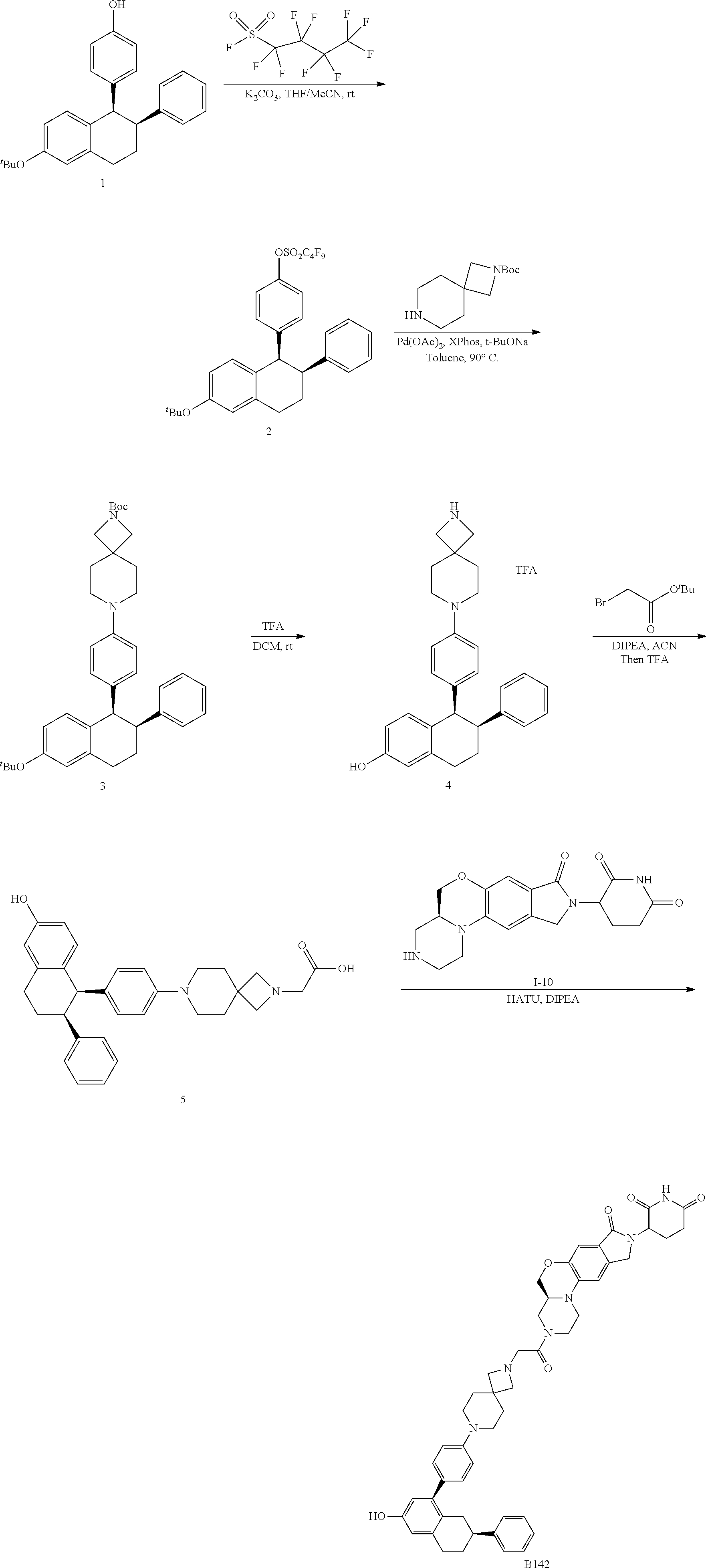

### Step 1:4-((1R,2S)-6-(tert-butoxy)-2-phenyl-1,2,3,4-tetrahydronaphthalen-1-yl)phenyl 4,4,4,4,4,4,4,4,4-nonafluoro-4112-buta-1,3-diyne-1-sulfonate

To a solution of 1 (2.0 g, 1 eq) and 1,1,2,2,3,3,4,4,4-nonafluorobutane-1-sulfonyl fluoride (3.24 g, 2.0 eq) in tetrahydrofuran (10 mL) and acetonitrile (10 mL) was added potassium carbonate (2.6 g, 3.5 eq). The reaction mixture was stirred at rt for 16 hours. TLC (petroleum ether: ethyl acetate=10:1) indicated the starting material was consumed completely and one new spot formed. The reaction mixture was concentrated under reduced pressure. The residue was purified by silica gel flash chromatography (petroleum ether:ethyl acetate=100:0 to 95:5). The desired compound 2 (3.5 g.>99% yield) was obtained as a colorless oil, ¹H NMR (400 MHZ, CDCl₃) δ 7.21-7.11 (m, 3H), 6.94-6.86 (m, 3H), 6.84-6.73 (m, 4H), 6.46 (d, J=8.8 Hz, 2H), 4.33 (d, J=5.2 Hz, 1H), 3.50-3.40 (m, 1H), 3.16-2.95 (m, 2H), 2.20-2.02 (m, 1H), 1.91-1.79 (m, 1H), 1.38 (s. 9H),

### Step 2: tert-butyl 9-(4-((1R,2S)-6-(tert-butoxy)-2-phenyl-1,2,3,4-tetrahydronaphthalen-1-yl)phenyl)-3,9-diazaspiro[3.5]undecane-3-carboxylate

A mixture of 2 (500 mg, 1.0 eq), tert-butyl 3,9-diazaspiro[3.5]undecane-3-carboxylate (345 mg, 2.0 eq), Pd(OAc)₂ (73 mg, 0.15 eq), XPhos (73 mg, 0.2 eq) and t-BuONa (257 mg, 3.5 eq) in tolune (10 mL) was degassed and purged with N_{2 3} times, and then the mixture was stirred at 90° C. for 16 h under N₂ atmosphere. LC-MS showed one main peak with desired MS was detected. TLC (PE:EA=10:1) indicated the starting material was consumed completely, and a new spot formed. The mixture was cooled, diluted with EA, filtered through Celite, the filter cake was washed with EA. The filtrate was concentrated. The residue was purified by silica gel flash chromatography (PE:EA=100:0 to 85:15). The desired product 3 (350 mg, 79% yield) was obtained as a colorless oil. LCMS (ESI) m/z: 581,17 [M+1].

### Step 3: (5R,6S)-5-(4-(3,9-diazaspiro[3.5]undecan-3-yl)phenyl)-6-phenyl-5,6,7,8-tetrahydronaphthalen-2-ol trifluoroacetate salt

To a solution of 3 (350 mg) in DCM (5 mL) was added TFA (2.5 mL). The reaction mixture was stirred overnight, then concentrated under reduced pressure. The residue was lyophilized, and compound 4 (333 mg) was obtained as a white solid. LCMS (ESI) m/z: 425.10 [M+1].

### Step 4:2-(7-(4-((1R,2S)-6-hydroxy-2-phenyl-1,2,3,4-tetrahydronaphthalen-1-yl)phenyl)-2,7-diazaspiro[3.5]nonan-2-yl)acetic acid

To a solution of 4 (30 mg, 1.0 equiv) in acetonitrile (6 mL) was added DIPEA (77.5 μL, 8.0 equiv) and tert-butyl 2-bromoacetate (9 μL, 1,1 equiv). The reaction mixture was stirred overnight. Then the solvent was removed, and the residue was purified by pre-HPLC. The fraction containing product was concentrated, followed by adding TFA, 2 h Later, TFA was removed under reduced pressure and compound 5 was obtained as a white solid after lyophilization. LCMS (ESI) m/z: 483.31 [M+1].

### Step 5:3-((S)-3-(2-(7-(4-((1R,2S)-6-hydroxy-2-phenyl-1,2,3,4-tetrahydronaphthalen-1-yl)phenyl)-2,7-diazaspiro[3.5]nonan-2-yl)acetyl)-8-oxo-1,2,3,4,4a,5,8,10-octahydro-9H-pyrazino[1′,2′: 4,5][1,4]oxazino[2,3-f]isoindol-9-yl)piperidine-2,6-dione (B142)

To a solution of 5 (12 mg, 1.0 equiv) in DMF (2.0 mL) was added DIPEA (21 μL, 6.0 equiv) and HATU (7.6 mg, 1.0 equiv), 10 Min later, intermediate 1-10 (11.3 mg, 1,2 equiv) was added and the reaction mixture was stirred for 10-15 min. Then the reaction mixture was immediately purified by pre-HPLC to obtain the title compound B142 as a white solid 14.2 mg (yield=69.6%). LC-MS (ESI) m/z: 821.36 [M+1].

### Compound B143:3-((S)-3-(2-(7-(4-((1R,2S)-6-hydroxy-2-phenyl-1,2,3,4-tetrahydronaphthalen-1-yl)phenyl)-2,7-diazaspiro[3.5]nonan-2-yl)-2-oxoethyl)-8-oxo-1,2,3,4,4a,5,8,10-octahydro-9H-pyrazino[1′,2′: 4,5][1,4]oxazino[2,3-f]isoindol-9-yl)piperidine-2,6-dione

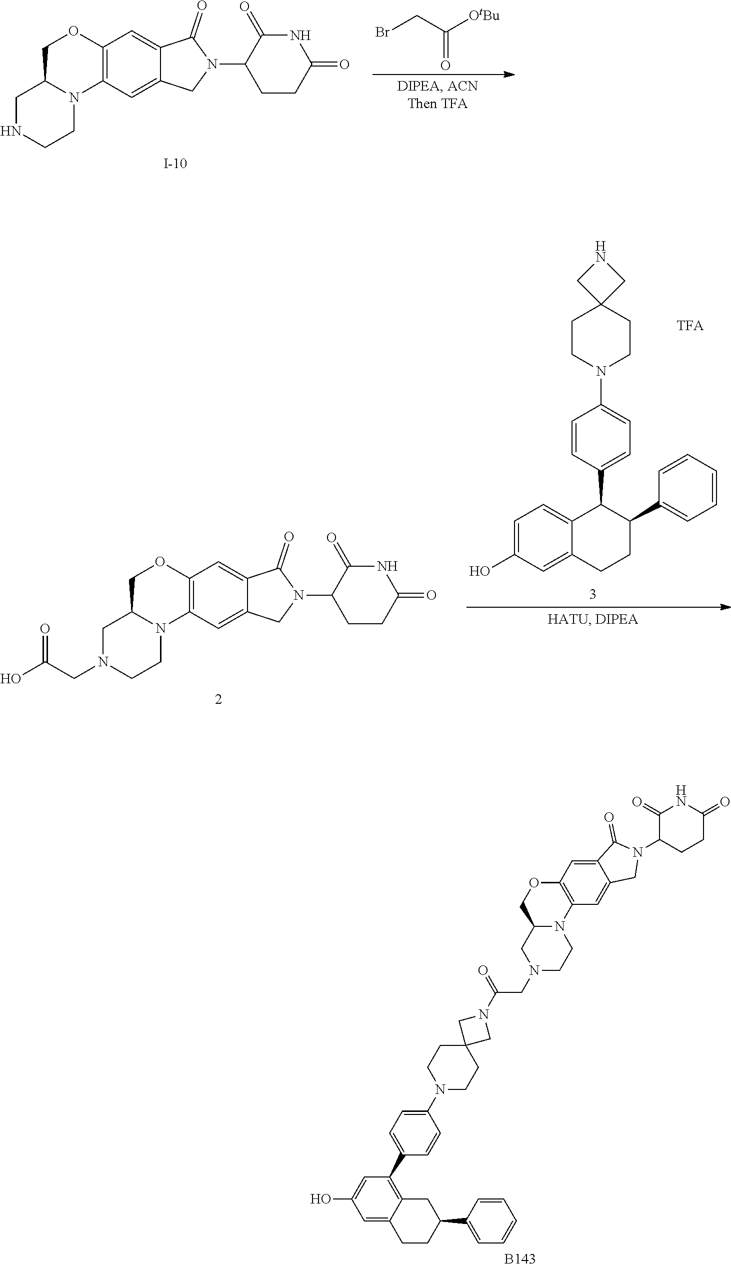

### Step 1:2-((4aS)-9-(2,6-dioxopiperidin-3-yl)-8-oxo-1,2,4a,5,9,10-hexahydro-8H-pyrazino[1′,2′: 4,5][1,4]oxazino[2,3-f]isoindol-3 (4H)-yl)acetic acid

To a solution of 1 (50 mg, 1.0 equiv) in acetonitrile (6 mL) was added DIPEA (148 μL, 8.0 equiv) and tert-butyl 2-bromoacetate (17.2 μL, 1,1 equiv). The reaction mixture was stirred overnight. Then the solvent was removed, and the residue was purified by pre-HPLC. The fraction containing product was concentrated, followed by adding TFA, 2 h Later, TFA was removed under reduced pressure and compound 2 was obtained as a white solid (45 mg, yield=80.4%) after lyophilization. LC-MS (ESI) m/z: 415.16 [M+1].

### Step 2:3-((S)-3-(2-(7-(4-((1R,2S)-6-hydroxy-2-phenyl-1,2,3,4-tetrahydronaphthalen-1-yl)phenyl)-2,7-diazaspiro[3.5]nonan-2-yl)-2-oxoethyl)-8-oxo-1,2,3,4,4a,5,8,10-octahydro-9H-pyrazino[1′,2′: 4,5][1,4]oxazino[2,3-f]isoindol-9-yl)piperidine-2,6-dione

To a solution of 2 (14 mg, 1.0 equiv) in DMF (2.0 mL) was added DIPEA (27.7 μL, 6.0 equiv) and HATU (10.1 mg, 1.0 equiv), 10 Min later, compound 2 (17.1 mg, 1,2 equiv) was added and the reaction mixture was stirred for 10-15 min. Then the reaction mixture was immediately purified by pre-HPLC to obtain the title compound B143 as a white solid 24 mg (yield=97%). LCMS (ESI) m/z: 821.36 [M+1].

The following compounds were prepared in a manner analogous to compounds B142 and B143.

TABLE 5
Compounds Prepared According to Compounds B142 and B143.

| Compound No. | LC-MS (M + H)⁺ |
|---|---|
| B142 | 821.36 |
| B143 | 821.51 |
| B144 | 781.47 |
| B145 | 781.27 |
| B146 | 849.46 |
| B147 | 849.45 |
| B152 | 807.35 |
| B153 | 807.38 |
| B154 | 807.40 |
| B155 | 807.34 |
| B156 | 807.36 |
| B157 | 807.34 |
| B160 | 821.35 |
| B161 | 821.37 |
| B162 | 834.41 |
| B168 | 806.20 |
| B169 | 806.22 |
| B170 | 806.34 |
| B171 | 820.18 |
| B172 | 808.15 |
| B204 | 767.3 |
| B205 | 793.32 |
| B206 | 807.38 |
| B207 | 767.31 |
| B208 | 793.41 |
| B209 | 807.4 |
| B212 | 806.4 |
| B213 | 820.49 |
| B214 | 820.33 |
| B218 | 821.39 |
| B219 | 821.39 |
| B220 | 767.35 |
| B221 | 821.39 |
| B222 | 793.36 |
| B223 | 823.41 |
| B244 | 781.3 |
| B245 | 767.9 |
| B246 | 768.3 |
| B247 | 780.8 |
| B248 | 768.3 |
| B249 | 781.3 |
| B250 | 767.30 |
| B251 | 793.20 |
| B252 | 821.20 |
| B253 | 820.30 |
| B254 | 821.30 |

### Compound B148:3-((S)-3-(2-(9-(5-(cis-6-hydroxy-2-phenyl-1,2,3,4-tetrahydronaphthalen-1-yl)pyridin-2-yl)-3,9-diazaspiro[5.5]undecan-3-yl)-2-oxoethyl)-8-oxo-1,2,3,4,4a,5,8,10-octahydro-9H-pyrazino[1′,2′: 4,5][1,4]oxazino[2,3-f]isoindol-9-yl)piperidine-2,6-dione

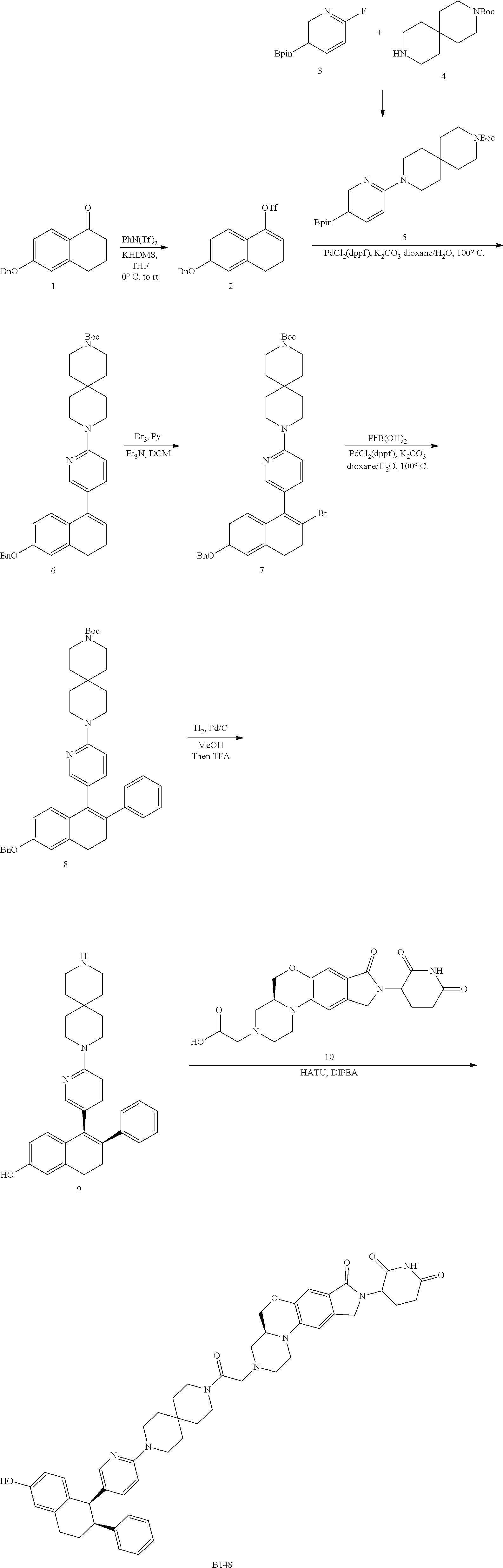

### Step 1:6-(benzyloxy)-3,4-dihydronaphthalen-1-yl trifluoromethanesulfonate

To a solution of 1 (12.6 g, 1.0 equiv) and PhN(Tf)₂ (21,4 g, 1,2 equiv) in THF (180 mL) was added KHDMS (0.5 M in toluene, 150 mL, 1.5 equiv) dropwise at 0° C. After the addition was completed, the reaction was warmed to rt and kept stirring for 4 h. Then quenched with ice water (150 mL) under ice bath. The result mixture was extracted with EA, and the combined EA layers was washed with brine, dried over Na₂SO₄, and concentrated. The crude product was purified by silica gel flash chromatography to give compound 2 as a light-yellow oil (15.1 g, yield=79%).

### Step 2: tert-butyl 9-(5-(4,4,5,5-tetramethyl-1,3,2-dioxaborolan-2-yl)pyridin-2-yl)-3,9-diazaspiro[5.5]undecane-3-carboxylate

To a solution of 3 (1.0 g, 1.0 equiv) and 4 (1.37 g, 1,2 equiv) in DMSO (10 mL) was added DIPEA (2.0 mL, 2.5 equiv). The reaction mixture was stirred at 100° C. overnight. Then the reaction mixture was cooled to rt, diluted with EA, washed with brine and concentrated to give a crude product, which was purified by silica gel flash chromatography to give compound 5 as a white powder (1.04 g, yield=51%). LC-MS (ESI) m/z: 376.32 [M+1].

### Step 3: tert-butyl 9-(5-(6-(benzyloxy)-3,4-dihydronaphthalen-1-yl)pyridin-2-yl)-3,9-diazaspiro[5.5]undecane-3-carboxylate

A mixture of 2 (381 mg, 1.0 equiv), 5 (500 mg, 1,1 equiv), PdCldppf (160 mg, 0.2 equiv) and K₂CO₃ (377 mg, 2.5 equiv) in dioxane/H₂O (8 mL/2 mL) was degassed and purged with N_{2 3} times, and then the mixture was stirred at 100° C. for 3 h under N₂ atmosphere. LC-MS showed one main peak with desired MS was detected. The reaction mixture was cooled to rt, extracted with EA, washed with brine, dried over Na₂SO₄ and concentrated. The result residue was purified by silica gel flash chromatography to give compound 6 as a white powder (1.04 g, yield=51%). LC-MS (ESI) m/z: 376.32 [M+1].

### Step 4: tert-butyl 9-(5-(6-(benzyloxy)-2-bromo-3,4-dihydronaphthalen-1-yl)pyridin-2-yl)-3,9-diazaspiro[5.5]undecane-3-carboxylate

To a solution of 6 (270 mg, 1.0 equiv) in DCM (25 mL) was added Et₃N (134 μL, 2.0 equiv), followed by adding Br3.Py (137 mg, 0.9 equiv) in potionwise at 0° C. The reaction mixture was kept stirring for 1 h, then diluted with DCM, washed with brine, dried over Na₂SO₄ and concentrated. The crude product was purified by silica gel flash chromatography to give compound 7 as a colorless oil (194 mg, yield=63%). LC-MS (ESI) m/z: 644.05 [M+1].

### Step 5: tert-butyl 9-(5-(6-(benzyloxy)-2-phenyl-3,4-dihydronaphthalen-1-yl)pyridin-2-yl)-3,9-diazaspiro[5.5]undecane-3-carboxylate

A mixture of 7 (194 mg, 1.0 equiv), PhB (OH)₂ (73.5 mg, 2.0 equiv), PdCl₂dppf (44 mg, 0.2 equiv) and K₂CO₃ (104 mg, 2.5 equiv) in dioxane/H₂O (10 mL/2 mL) was degassed and purged with N₂3 times, and then the mixture was stirred at 100° C. overnight under N₂ atmosphere. LC-MS showed one main peak with desired MS was detected. The reaction mixture was cooled to rt, extracted with EA, washed with brine, dried over Na₂SO₄ and concentrated. The result residue was purified by silica gel flash chromatography to give compound 8 as a white solid (175 mg, yield=91%). LC-MS (ESI) m/z: 641.93 [M+1].

### Step 6: (5S,6S)-5-(6-(3,9-diazaspiro[5.5]undecan-3-yl)pyridin-3-yl)-6-phenyl-5,6,7,8-tetrahydronaphthalen-2-ol

A mixture of 8 (50 mg, 1.0 equiv) and Pd/C(10% Pd/C powder, 20 mg, 40%) in MeOH was degassed and purged with N_{2 3} times, and then the mixture was stirred at rt overnight. The mixture was filtered throught Celite, washed with DCM, and the filtration was concentrated. The result residue was dissolved in DCM (4 mL) and TFA (2 mL) was added, 30 Min later, concentrated under reduced pressure to remove the solvent and the desired product 9 was obtained, which was further lyophilized to give a gray solid. LC-MS (ESI) m/z: 454.09 [M+1]. Step 7:3-((S)-3-(2-(9-(5-((1S,2S)-6-hydroxy-2-phenyl-1,2,3,4-tetrahydronaphthalen-1-yl)pyridin-2-yl)-3,9-diazaspiro[5.5]undecan-3-yl)-2-oxoethyl)-8-oxo-1,2,3,4,4a,5,8,10-octahydro-9H-pyrazino[1′,2′: 4,5][1,4]oxazino[2,3-f]isoindol-9-yl)piperidine-2,6-dione

To a solution of 10 (5.0 mg, 1.0 equiv) in DMF (2.0 mL) was added DIPEA (9.9 μL, 6.0 equiv) and HATU (3.6 mg, 1.0 equiv), 10 Min later, compound 9 (6.5 mg, 1,2 equiv) was added and the reaction mixture was stirred for 10-15 min. Then the reaction mixture was immediately purified by pre-HPLC to obtain the title compound B148 as a white solid 6.9 mg (yield=76%). LCMS (ESI) m/z: 850.42 [M+1].

### Compound B149:3-((S)-3-(2-(9-(5-(cis-6-hydroxy-2-phenyl-1,2,3,4-tetrahydronaphthalen-1-yl)pyrimidin-2-yl)-3,9-diazaspiro[5.5]undecan-3-yl)-2-oxoethyl)-8-oxo-1,2,3,4,4a,5,8,10-octahydro-9H-pyrazino[1′,2′: 4,5][1,4]oxazino[2,3-f]isoindol-9-yl)piperidine-2,6-dione

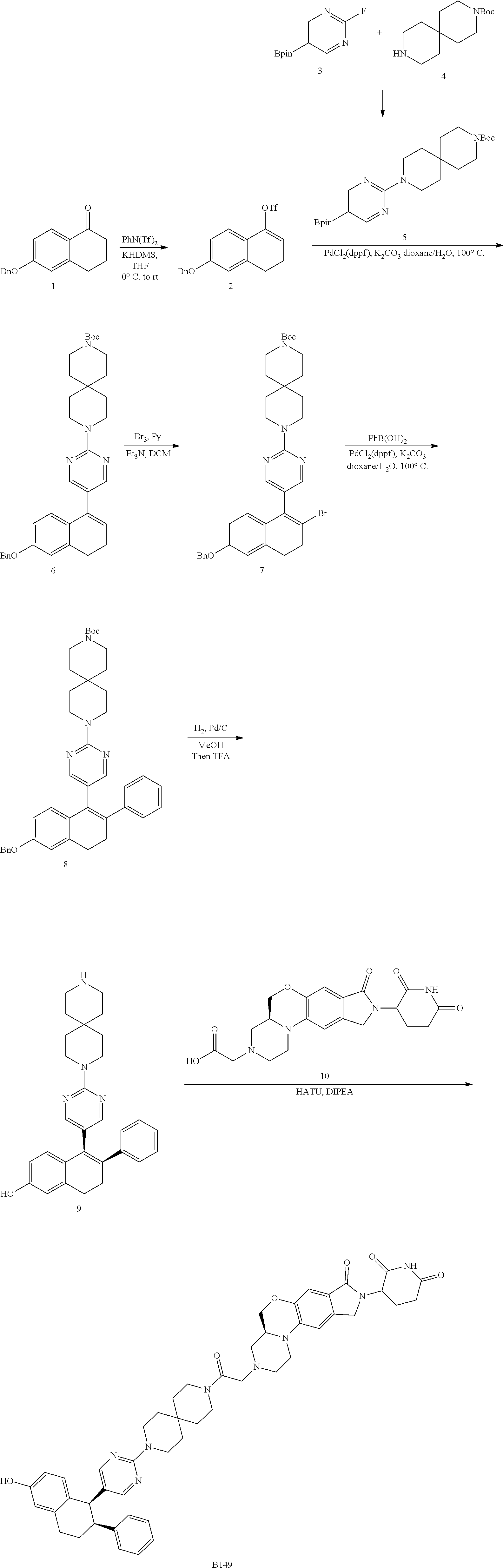

Compound B149 was made using a similar procedure for making compound B148. LC-MS (ESI) m/z: 851.38 [M+1].

### Compound B173: 3-(4aS)-3-(8-(4-(1R,2S)-6-hydroxy-2-phenyl-1,2,3,4 tetrahydronaphthalen-1-yl)phenyl)-2-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

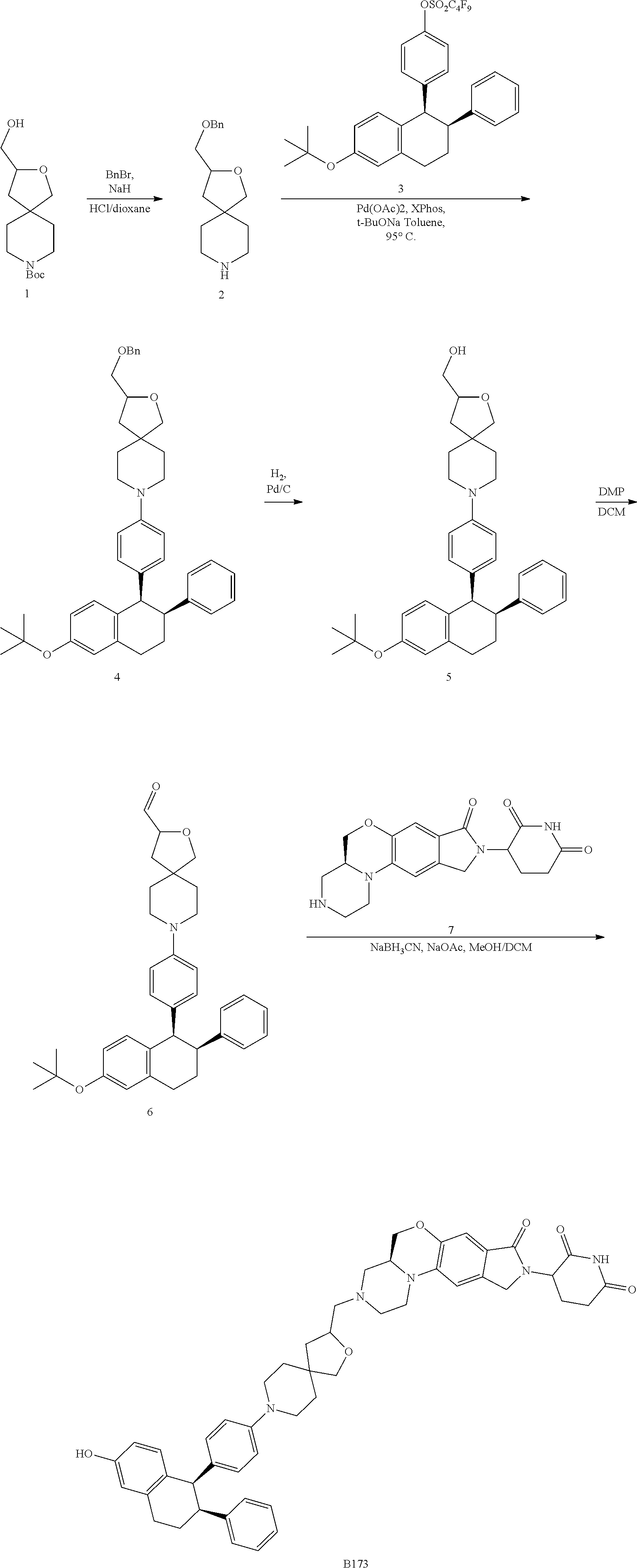

### Step 1:3-((benzyloxy)methyl)-2-oxa-8-azaspiro[4.5]decane

To a solution of 1 (2 g, 1.0 equiv) in DMF was added slowly added NaH (60%, 590 mg, 2.0 equiv) at 0° C. 30 min Later, BnBr (1.05 mL, 1,2 equiv) was added and the reaction was kept stirring overnight. The next day, quenched with ice-water and extracted with EA. The combined organic layer was washed with brine, dried over Na₂SO₄, and concentrated under reduced pressure. The result mixture was purified by silica gel flash chromatography to give a colorless oil 2.0 g. which was treated with 4M HCl/dioxane to give compound 2. LC-MS (ESI) m/z: 262.06 [M+1].

### Step 2:3-((benzyloxy)methyl)-8-(4-(1R,2S)-6-(tert-butoxy)-2-phenyl-1,2,3,4-tetrahydronaphthalen-1-yl)phenyl)-2-oxa-8-azaspiro[4.5]decane

A mixture of 2 (341 mg, 1.5 equiv), 3 (500 mg, 1.0 equiv), Pd(OAc)₂ (34.3 mg, 0.2 equiv), XPhos (73 mg, 0.2 equiv) and t-BuONa (330 mg, 4.5 equiv) in toluene (18 mL) was degassed with N₂ and then kept stirred at 95° C. overnight. Next, the reaction was cooled to rt, filtered through Celite and the filter cake was washed with DCM. The filtration was concentrated under reduced pressure and the result residue was purified by silica gel flash chromatography to give product 4 (382 mg) as a sticky white silid. LC-MS (ESI) m/z: 616.25 [M+1].

### Step 3: (8-(4-((1R,2S)-6-(tert-butoxy)-2-phenyl-1,2,3,4-tetrahydronaphthalen-1-yl)phenyl)-2-oxa-8-azaspiro[4.5]decan-3-yl)methanol

A mixture of 4 (382 mg, 1.0 equiv) and Pd/C(10%, 382 mg) in MeOH/EA was degassed with H₂ and stirred under H₂ atmosphere for 2 days. Then the reaction mixture was filtered and the filtration was concentrated under reduced pressure. The result residue was purified by silica gel flash chromatography to give product 5 as a colorless oil. LC-MS (ESI) m/z: 526.21 [M+1].

### Step 4:8-(4-((1R,2S)-6-(tert-butoxy)-2-phenyl-1,2,3,4-tetrahydronaphthalen-1-yl)phenyl)-2-oxa-8-azaspiro[4.5]decane-3-carbaldehyde

To a solution of 5 (50 mg, 1.0 equiv) in DCM (8 mL) was added DMP (168.5 mg, 1.7 equiv). The reaction mixture was stirred at rt for 2 h, then diluted with DCM, washed with brine, dried over Na₂SO₄, and concentrated under reduced pressure. The result residue was purified by silica gel flash chromatography to provide compound 6 as a purple solid 44 mg. LC-MS (ESI) 542.29 m/z: [M+H₂O+1].

### Step 5:3-((4aS)-3-((8-(4-((1R,2S)-6-hydroxy-2-phenyl-1,2,3,4-tetrahydronaphthalen-1-yl)phenyl)-2-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (B173)

To a solution of 6 (14 mg, 1.5 equiv) and 7 (6.4 mg, 1.0 equiv) in MeOH/DCM (3 mL/1 mL) was added NaBH₃CN in potions (6.6 mg, 6.0 equiv), 24 h Later, the solvent was removed, and the result residue was purified by pre-HPLC, which was further treated with TFA togive the final compound B173 as a white solid. LC-MS (ESI) m/z: 808.15 [M+1].

Compounds B172, B174. B175 were prepared using the similar procedure for making B173.

### Compound B177:3-((4aS)-3-((8-(4-((1R,2S)-6-hydroxy-2-phenyl-1,2,3,4-tetrahydronaphthalen-1-yl)phenyl)-1-oxa-8-azaspiro[4.5]decan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

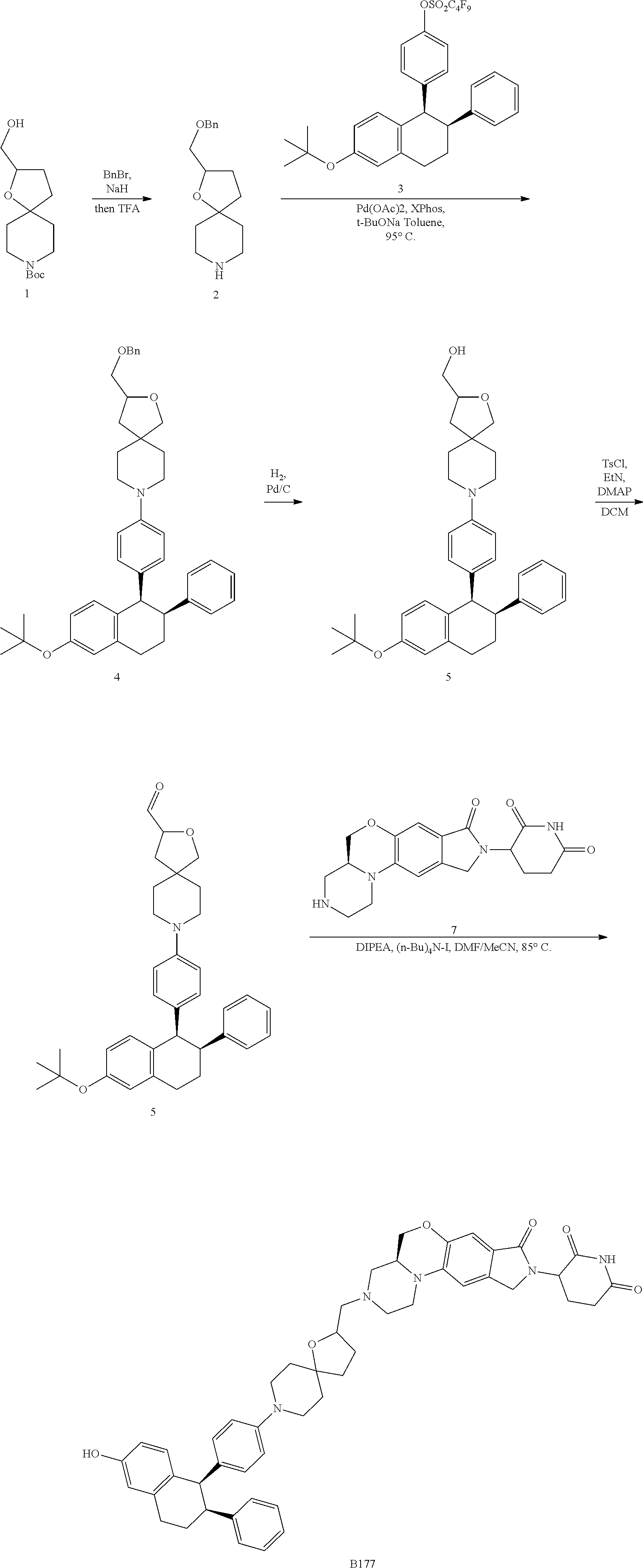

### Step 1:2-((benzyloxy)methyl)-1-oxa-8-azaspiro[4.5]decane

To a solution of 1 (1 g, 1.0 equiv) in DMF was added slowly added NaH (60%, 295 mg, 2.0 equiv) at 0° C. 30 min Later, BnBr (526 μL, 1,2 equiv) was added and the reaction was kept stirring overnight. The next day, quenched with ice-water and extracted with EA. The combined organic layer was washed with brine, dried over Na₂SO₄, and concentrated under reduced pressure. The result mixture was purified by silica gel flash chromatography to give a light-yellow oil 1.08 g, which was treated with 4M HCl/dioxane to give compound 2 as a brown oil 1,1 g. LC-MS (ESI) m/z: 262.06 [M+1].

### Step 2:2-((benzyloxy)methyl)-8-(4-((1R,2S)-6-(tert-butoxy)-2-phenyl-1,2,3,4-tetrahydronaphthalen-1-yl)phenyl)-1-oxa-8-azaspiro[4.5]decane

A mixture of 2 (341 mg, 1.5 equiv), 3 (500 mg, 1.0 equiv), Pd(OAc)₂ (34.3 mg, 0.2 equiv), XPhos (73 mg, 0.2 equiv) and t-BuONa (330 mg, 4.5 equiv) in toluene (18 mL) was degassed with N₂ and then kept stirred at 95° C. overnight. Next, the reaction was cooled to rt, filtered through Celite and the filter cake was washed with DCM. The filtration was concentrated under reduced pressure and the result residue was purified by silica gel flash chromatography to give product 4 (387 mg) as a sticky white silid. LC-MS (ESI) m/z: 616.26 [M+1].

### Step 3: (8-(4-((1R,2S)-6-(tert-butoxy)-2-phenyl-1,2,3,4-tetrahydronaphthalen-1-yl)phenyl)-1-oxa-8-azaspiro[4.5]decan-2-yl)methanol

A mixture of 4 (387 mg, 1.0 equiv) and Pd/C(10%, 387 mg) in MeOH was degassed with H₂ and stirred under H₂ atmosphere for 24 h. Then the reaction mixture was filtered, and the filtration was concentrated under reduced pressure. The result residue was purified by silica gel flash chromatography to give product 5 as a white foam 150 mg. LC-MS (ESI) m/z: 526.19 [M+1].

### Step 4: (8-(4-((1R,2S)-6-(tert-butoxy)-2-phenyl-1,2,3,4-tetrahydronaphthalen-1-yl)phenyl)-1-oxa-8-azaspiro[4.5]decan-2-yl)methyl 4-methylbenzenesulfonate

To a solution of 5 (150 mg, 1.0 equiv), Et₃N (119 μL, 3.0 equiv) and DMAP (6.9 mg, 0.2 equiv) in DCM was added TsCl (81.5 mg, 1.5 equiv). The reaction mixture was stirred at rt overnight, then quenched with water, extracted with EA. The combined organic layer was washed with brine, dried over Na₂SO₄, and concentrated under reduced pressure. The result residue was purified by silica gel flash chromatography to give product 6 as a white foam 211 mg. LC-MS (ESI) m/z: 680.25 [M+1].

### Step 5:3-((4aS)-3-((8-(4-((1R,2S)-6-hydroxy-2-phenyl-1,2,3,4-tetrahydronaphthalen-1-yl)phenyl)-1-oxa-8-azaspiro[4.5]decan-2-yl)methyl)-8-oxo-1,2,3,4,4a,5,8,10-octahydro-9H-pyrazino[1,2′: 4,5][1,4]oxazino[2,3-f]isoindol-9-yl)piperidine-2,6-dione

To a mixture of 6 (17.4 equiv, 0.8 equiv), 7 (15 mg, 1.0 equiv) and (n-Bn) 4N-I (29 mg, 2.5 equiv) in DMF/MeCN (2 mL/2 mL) was added DIPEA (111 μL, 20 equiv). The reaction mixture was stirred at 85° C. for 2 days, then purified by pre-HPLC to give the pure intermediate, which was treated with TFA to provide the final compound B177. LC-MS (ESI) m/z: 808.10 [M+1]. Compounds B176 and B178 were prepared using the similar procedure for making B177.

### Compound B366. (3S)-3-(1′-((9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione

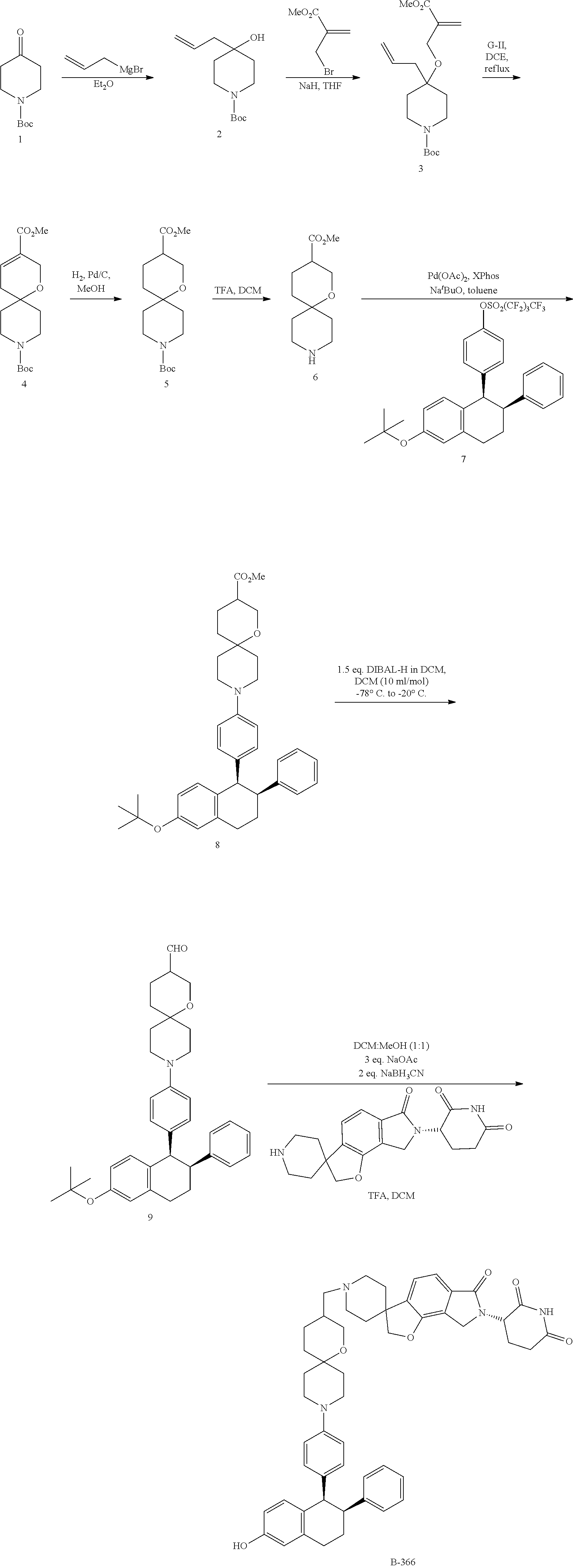

### Step 1: tert-butyl 4-allyl-4-hydroxypiperidine-1-carboxylate

Allyl magnesium bromide (1M sol, in Et₂O, 26 mL) was added at 0° C. to a solution of N-Boc-4-piperidone (1, 4.03 g, 20 mmol) in Et₂O (80 mL). It was stirred for 10 min. The reaction mixture was warmed to room temperature and stir for 4 h. Followed by quenching by addition of sat, aq. NH₄Cl. It was then extract with EtOAc. The organic phase was separated and washed twice with water then brine, then dried over sodium sulfate, filtered and concentrated in vacuo. The crude mixture was purified using column chromatography on silica gel (0% to 100% ethyl acetate in hexanes. The desired compound 2 (4.84 g, ˜ 90% yield) was obtained as a colorless oil.

¹H NMR: (400 MHZ, CDCl₃) δ 5.77-5.94 (m, 1H), 5.19 (dd, J=10.4, 1.8 Hz, 1H), 5.14 (dd, J=17.1. 1.9 Hz, 1H), 3.81 (dt, J=13,4, 3.3 Hz, 2H), 3.08-3,24 (m, 2H), 2.23 (d. J=7.6 Hz, 2H), 1.53 (dd. J=10.4, 4.8 Hz, 4H), 1.46 (s, 9H),

### Step 2: tert-butyl 4-allyl-4-((2-(methoxycarbonyl) allyl)oxy)piperidine-1-carboxylate

A 60% oil dispersion of sodium hydride (0.438 g, 1,2 eq) was added to a solution of tert-butyl 4-allyl-4-hydroxypiperidine-1-carboxylate (2, 2.2 g, 1 eq) in anhydrous DMF (10 mL/mmol) and the mixture cooled to 0° C. The mixture was warmed to room temperature over 1 hour and methyl 2-(bromomethyl) acrylate (1.63 g, 1 eq) in DMF was added dropwise to the solution over 5 minutes. The mixture was stirred for 12 h. The reaction mixture cooled down to 0° C. a saturated solution of ammonium chloride was added to the reaction mixture and the mixture was diluted with ethyl acetate. The organic phase was separated and washed twice with water then brine, then dried over sodium sulfate, filtered and concentrated in vacuo. The crude mixture was purified using column chromatography on silica gel (0% to 100% ethyl acetate in hexanes, Rf: 0.3; 30% EA/Hx). The desired compound 3 was obtained as a colorless oil. Yield: 60-70%

### Step 3:9-(tert-butyl) 3-methyl 1-oxa-9-azaspiro[5.5]undec-3-ene-3,9-dicarboxylate

tert-butyl 4- {[2-(methoxycarbonyl)prop-2-en-1-yl]oxy}-4-(prop-2-en-1-yl)piperidine-1-carboxylate (3, 340 mg, 1 eq) in anhydrous 1,2-dichloroethane (20 mL/mmol) was combined with G-II (0.05 eq) and the mixture was heated at 50° C. for 4 h. The mixture was cooled to room temperature and quenched by passing air. It was then filtered and evaporated and purified by flash. tert-butyl 3-oxo-1-oxa-9-azaspiro[5.5]undecane-9-carboxylate (4) was obtained as an oil. Yield: ˜80%

### Step 4:9-(tert-butyl) 3-methyl 1-oxa-9-azaspiro[5.5]undecane-3,9-dicarboxylate

Pd/C(100 mg, 10% wt.) was added to a solution of compound 4 (1 gm, 3.31 mmol) in MeOH (33 mL, 10 mL/mmol). The reaction mixture was degassed with H₂ and stirred under a H₂ atmosphere for 12 h at room temperature. The mixture was then filtered through celite and washed with MeOH. Concentration under reduced pressure followed by purification by flash chromatography (O to 100% ethyl acetate in hexanes) gave the desired compound 5 in 60% yield.

### Step 5: methyl 1-oxa-9-azaspiro[5.5]undecane-3-carboxylate

To a solution of 5 (300 mg) in DCM (5 mL) was added TFA (2.5 mL). The reaction mixture was stirred overnight, then concentrated under reduced pressure and used for the next steps without further purification.

### Step 6: methyl 9-(4-((1R,2S)-6-(tert-butoxy)-2-phenyl-1,2,3,4-tetrahydronaphthalen-1-yl)phenyl)-1-oxa-9-azaspiro[5.5]undecane-3-carboxylate

A mixture of 4-((1R,2S)-6-(tert-butoxy)-2-phenyl-1,2,3,4-tetrahydronaphthalen-1-yl)phenyl 1,1,2,2,3,3,4,4,4-nonafluorobutane-1-sulfonate (654 mg, 1.0 eq), 6 (319 mg, 1.5 eq), Pd(OAc)₂ (73 mg, 0.15 eq), XPhos (73 mg, 0.2 eq) and t-BuONa (257 mg, 3.5 eq) in tolune (20 mL) was degassed and purged with N₂ three times, and then the mixture was stirred at 90° C. for 3 h under N₂ atmosphere. LC-MS showed one main peak with desired MS was detected. TLC (PE:EA=10:1) indicated the starting material was consumed completely, and a new spot formed. The mixture was cooled, diluted with EA, filtered through Celite, the filter cake was washed with EA. The filtrate was concentrated. The residue was purified by silica gel flash chromatography (0% to 50% ethyl acetate in hexanes). The desired product 7 (340 mg, 60% yield) was obtained as a colorless oil. LCMS (ESI) m/z: 568.30 [M+1].

### Step 7:9-(4-((1R,2S)-6-(tert-butoxy)-2-phenyl-1,2,3,4-tetrahydronaphthalen-1-yl)phenyl)-1-oxa-9-azaspiro[5.5]undecane-3-carbaldehyde

To a solution of 7 (567 mg, 1.0 mmol, 10 mL/mmol) in DCM at −78° C. 1.5 mL of DIBAL-H (1.0 M in DCM) was added dropwise. Then, the temperature was slowly increased to −20° C. and stirred for 6 h. After that, the reaction was slowly quenched with satd. Na₂SO₄ at 0° C. and was then filtered and washed several times with EtOAc. Purification by flash chromatography to obtain the desired product (8) LCMS (ESI) m/z: 55.20 [M+18].

### Step 8: (3S)-3-(1′-((9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione

To a mixture of compound 8 (53 mg, 0.1 eq.) in methanol (5 mL) and dichloromethane (5 mL) was added(S)-3-(6-oxo-6,8-dihydro-2H,7H-spiro[furo[2,3-e]isoindole-3,4′-piperidin]-7-yl)piperidine-2,6-dione (35 mg, 0.1 eq.), and AcONa (24 mg, 0.3 eq.). The mixture was stirred at 25° C. for 20 mins, then sodium cyanoborohydride (0.2 mL, 0.2 eq., 1M in THF) was added and the mixture was further stirred for 10 mins. LCMS showed the reaction was complete. Next, the reaction mixture was concentrated under reduced pressure.

The crude reaction mixture was dissolved in DCM (10 mL), and then TFA (3 mL) was added, 1 h later, LC-MS showed starting material was consumed completely, and a new peak with desired MS was detected. Next, the reaction mixture was quenched with water and concentrated under reduced pressure. It was purified by pre-HPLC, and the desired product (B366) was obtained as white solid. LCMS (ESI) m/z: 821.40 [M+1].

### Compound B391:3-((S)-2-((9-(4-((1R,2S)-6-hydroxy-2-phenyl-1,2,3,4-tetrahydronaphthalen-1-yl)phenyl)-3,9-diazaspiro[5.5]undecan-3-yl)methyl)-6-oxo-2,3,6,8-tetrahydro-7H-[1,4]dioxino[2,3-f]isoindol-7-yl)piperidine-2,6-dione

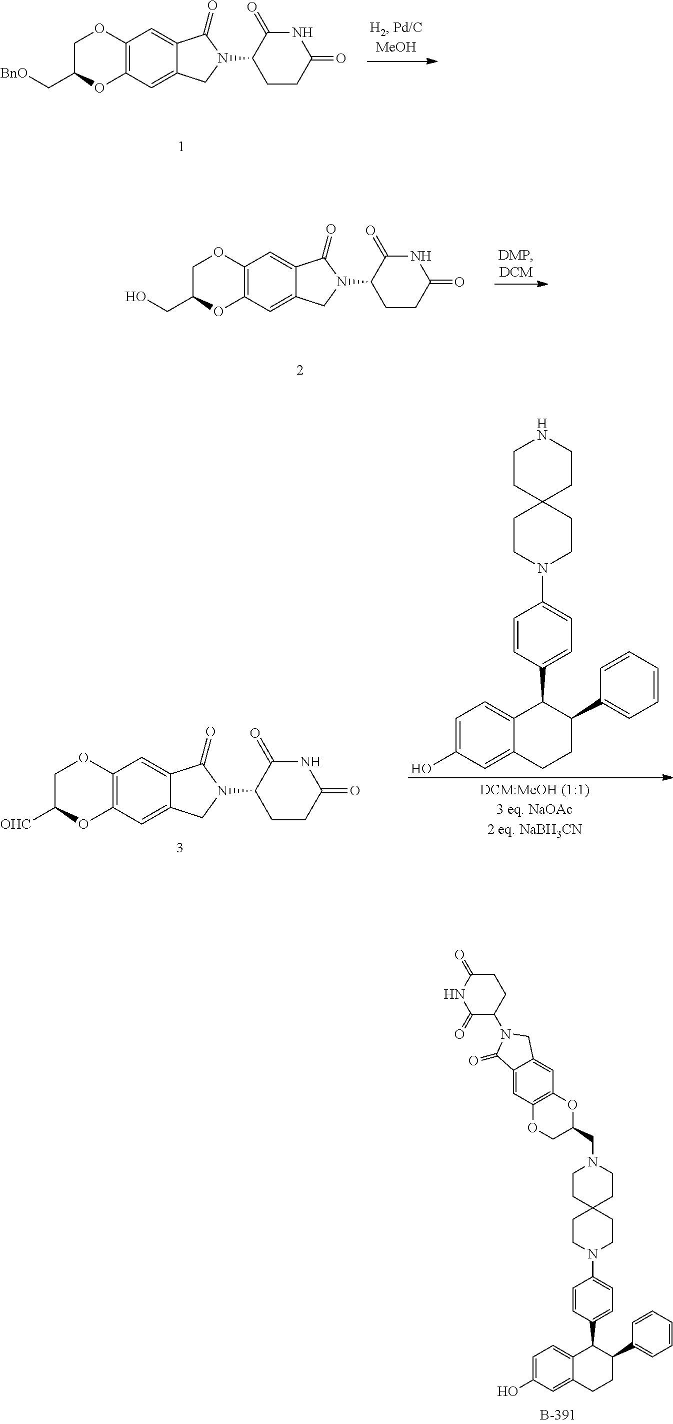 [SMILES: O=C1CC[C@H](N2Cc3cc4c(cc3C2=O)OC[C@H](COCc2ccccc2)O4)C(=O)N1 ; O=C1CC[C@H](N2Cc3cc4c(cc3C2=O)OC[C@H](CO)O4)C(=O)N1 ; O=C[C@H]1COc2cc3c(cc2O1)CN([C@H]1CCC(=O)NC1=O)C3=O ; Oc1ccc2c(c1)CC[C@H](c1ccccc1)[C@@H]2c1ccc(N2CCC3(CCNCC3)CC2)cc1 ; O=C1CCC(N2Cc3cc4c(cc3C2=O)OC[C@H](CN2CCC3(CC2)CCN(c2ccc([C@@H]5c6ccc(O)cc6CC[C@@H]5c5ccccc5)cc2)CC3)O4)C(=O)N1]

### Step 1: (S)-3-((S)-2-(hydroxymethyl)-6-oxo-2,3,6,8-tetrahydro-7H-[1,4]dioxino[2,3-f]isoindol-7-yl)piperidine-2,6-dione

Pd/C(42 mg, 10% wt.) was added to a solution of compound 1 (422 mg, 1 mmol) in MeOH (30 mL). The reaction mixture was degassed with H₂ and stirred under a H₂ atmosphere for 2 h at room temperature. The mixture was then filtered through celite, concentration under reduced pressure and used as a crude for the next steps.

### Step 2 (R)-7-((S)-2,6-dioxopiperidin-3-yl)-6-oxo-2,3,7,8-tetrahydro-6H-[1,4]dioxino[2,3-flisoindole-2-carbaldehyde

To a solution of 2 (1.0 eq., 330 mg) in DCM (10 mL) was added DMP (1,2 eq.) at 0° C. and stirred it for 30 mins. TLC (ethyl acetate: hexane=1:1) showed reaction was complete. The reaction mixture was then diluted with DCM, washed with brine, dried over and concentrated under reduced pressure. The residue was purified by silica gel chromatography to give 3 in (70% yield). LC/MS (ESI) m/z: 348.12 (M+H₂O).

### Step 3 3-((S)-2-((9-(4-((1R,2S)-6-hydroxy-2-phenyl-1,2,3,4-tetrahydronaphthalen-1-yl)phenyl)-3,9-diazaspiro[5.5]undecan-3-yl)methyl)-6-oxo-2,3,6,8-tetrahydro-7H-[1,4]dioxino[2,3-f]isoindol-7-yl)piperidine-2,6-dione

To a mixture of compound 3 (33 mg, 0.1 eq.) in methanol (4 mL) and dichloromethane (4 mL) was added (5R,6S)-5-(4-(3,9-diazaspiro[5.5]undecan-3-yl)phenyl)-6-phenyl-5,6,7,8-tetrahydronaphthalen-2-ol (45 mg, 0.1 eq.), and AcONa (24 mg, 0.3 eq.). The mixture was stirred at 25° C. for 20 mins, then sodium cyanoborohydride (0.2 mL, 0.2 eq., 1M in THF) was added and the mixture was further stirred for 10 mins. LCMS showed the reaction was complete. Next, the reaction mixture was concentrated under reduced pressure. It was purified by pre-HPLC, and the desired product (B-391) was obtained as white solid. LCMS (ESI) m/z: 767.28 [M+1].

The following compounds were prepared using the similar procedure for making B391:

TABLE 6
Compounds Prepared According to Compounds B391

| Compound No. | (M + H)⁺ |
|---|---|
| B387 | 767.19 |
| B388 | 739.19 |
| B392 | 767.19 |
| B393 | 739.19 |

### Compound B226. (3S)-3-((5aR)-7-((8-(4-((1R,2S)-6-hydroxy-2-phenyl-1,2,3,4-tetrahydronaphthalen-1-yl)phenyl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

### Compound B227. (3S)-3-((4aS)-3-((8-(4-((1R,2S)-6-hydroxy-2-phenyl-1,2,3,4-tetrahydronaphthalen-1-yl)phenyl)-1-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

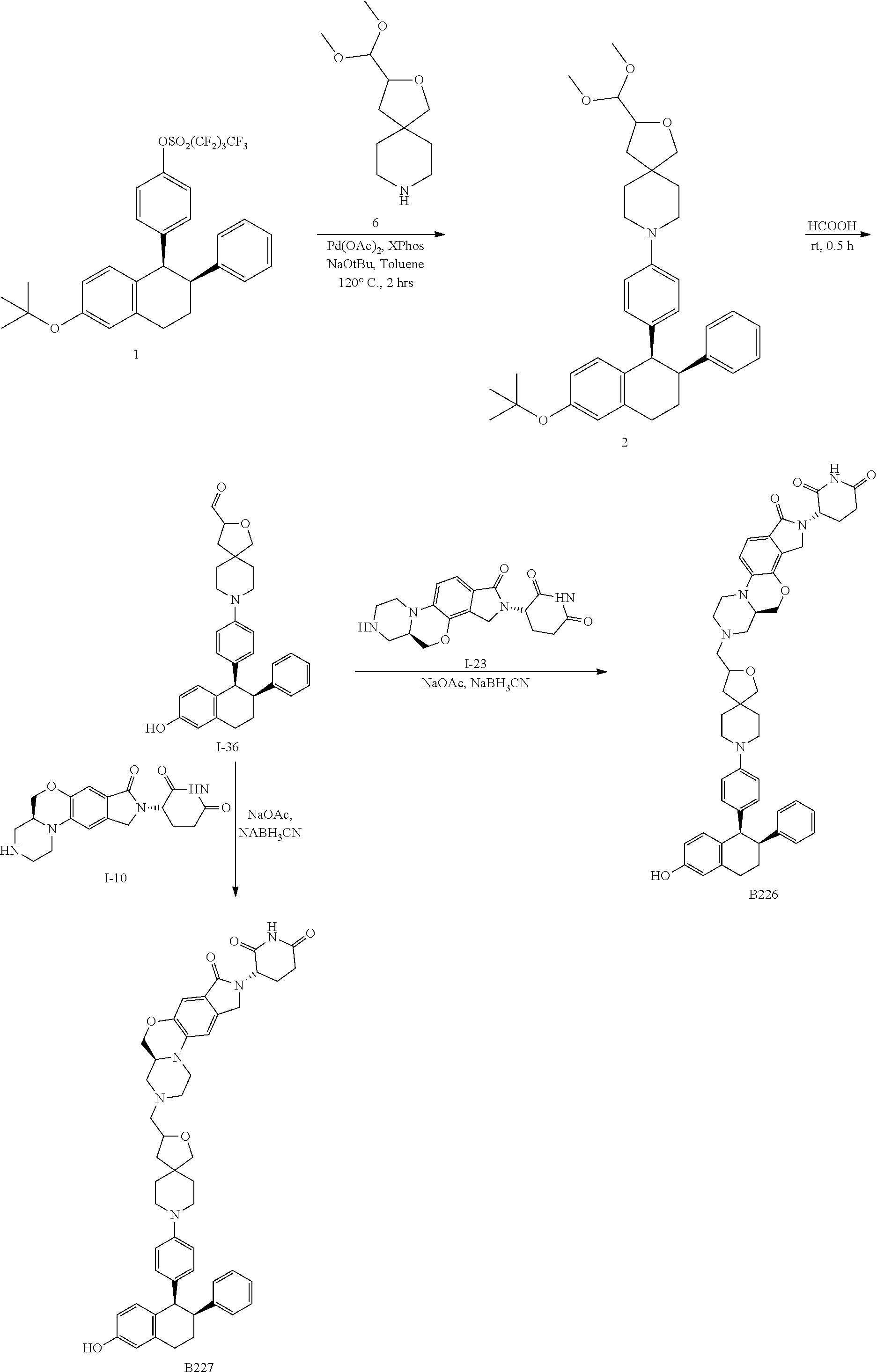

### Step 1:8-(4-((1R,2S)-6-(tert-butoxy)-2-phenyl-1,2,3,4-tetrahydronaphthalen-1-yl)phenyl)-3-(dimethoxymethyl)-1-oxa-8-azaspiro[4.5]decane

To a mixture of 4-((1R,2S)-6-(tert-butoxy)-2-phenyl-1,2,3,4-tetrahydronaphthalen-1-yl) phenyl 1,1,2,2,3,3,4,4,4-nonafluorobutane-1-sulfonate (100 mg, 0.15 mmol, 1.0 eq), 3-(dimethoxymethyl)-1-oxa-8-azaspiro[4.5]decane (39 mg, 0.18 mmol, 1.0 eq), t-BuONa (44 mg. 0.46 mmol, 3.0eq) and Xphos (7 mg, 0.02 mmol, 0.1 eq) in toluene (5 mL) was added Pd(OAc)₂ (13 mg, 0.02 mmol, 0.1 eq), then stirred at 120° C. for 2 hours. LC-MS showed the reaction was completed. The reaction mixture was cooled to rt, the mixture was diluted with water and washed with EtOAc, the organic phase was dried with Na₂SO₄ and concentrated under vacuum, the residue was purified by SiO₂ column chromatography (EtOAc:PE=1:20) to afford 8-(4-((1R,2S)-6-(tert-butoxy)-2-phenyl-1,2,3,4-tetrahydronaphthalen-1-yl)phenyl)-3-(dimethoxymethyl)-1-oxa-8-azaspiro[4.5]decane (66 mg, 77%) as a yellow oil. LC-MS purity: 62.1% (UV at 254 nm), LC-MS: 570.3 [M+H]⁺.

### Step 2:8-(4-((1R,2S)-6-hydroxy-2-phenyl-1,2,3,4-tetrahydronaphthalen-1-yl)phenyl)-1-oxa-8-azaspiro[4.5]decane-3-carbaldehyde

To a mixture of 7-(4-((1R,2S)-6-(tert-butoxy)-2-phenyl-1,2,3,4-tetrahydronaphthalen-1-yl)phenyl)-2-(dimethoxymethyl)-7-azaspiro[3.5]nonane (66 mg, 0.11 mmol, 1 eq) in HCOOH (3 mL) was stirred at 25° C. for 0.5 hour. The mixture was concentrated to give crude product 7-(4-((1R,2S)-6-hydroxy-2-phenyl-1,2,3,4-tetrahydronaphthalen-1-yl)phenyl)-7-azaspiro[3.5]nonane-2-carbaldehyde (48 mg, 90%) as a colorless liquid. LC-MS purity: 100% (UV at 254 nm), LC-MS: 468.2 [M+H]⁺.

### Step 36-3&4: (3S)-3-((5aR)-7-((8-(4-((1R,2S)-6-hydroxy-2-phenyl-1,2,3,4-tetrahydronaphthalen-1-yl)phenyl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

To a solution of I-23 (1,2 equiv) in Methanol:DCM (4:1) was added NaOAc (2 equiv) and the mixture was stirred at room temperature for 5 mins. The aldehyde I-36 (1 equiv) in MeOH:DCM (4:1) was then added to the reaction mixture and stirred for another 10 mins. Sodiumcyanoborohydride (2 equiv) and Acetic acis was then added to the reaction sequentially and stirred for another 15 minutes. UPLC chromatography showed the completion of the reaction, and the product was purified using preparative HPLC. LC/MS (ESI) m/z: 808.4 (M+H).

(3S)-3-((4aS)-3-((8-(4-((1R,2S)-6-hydroxy-2-phenyl-1,2,3,4-tetrahydronaphthalen-1-yl)phenyl)-1-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione: LC/MS (ESI) m/z: 808.4 (M+H).

### Compound 226X. (S)-3-((R)-7-(((R)-8-(4-((1R,2S)-6-hydroxy-2-phenyl-1,2,3,4-tetrahydronaphthalen-1-yl)phenyl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

### Compound 226Y. (S)-3-((R)-7-(((S)-8-(4-((1R,2S)-6-hydroxy-2-phenyl-1,2,3,4-tetrahydronaphthalen-1-yl)phenyl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

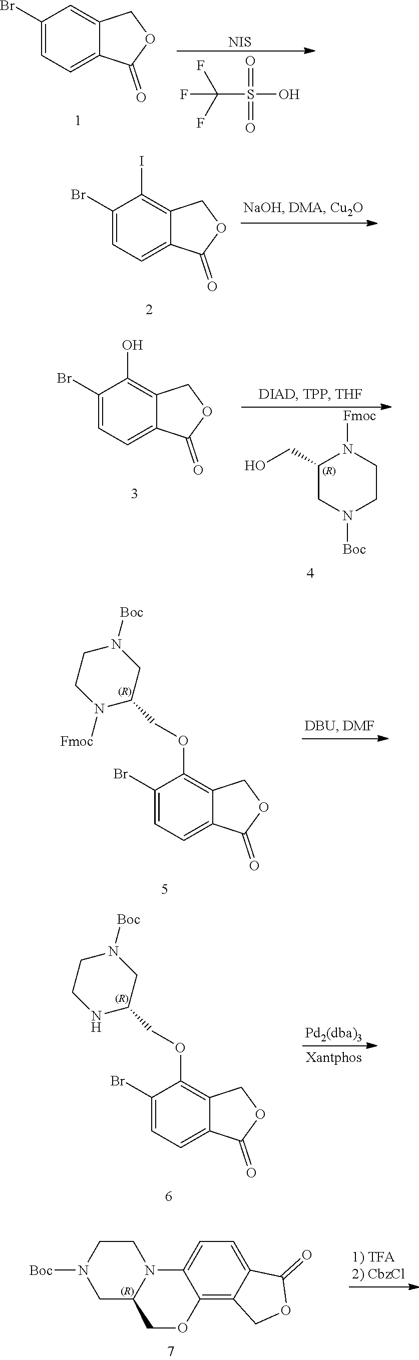

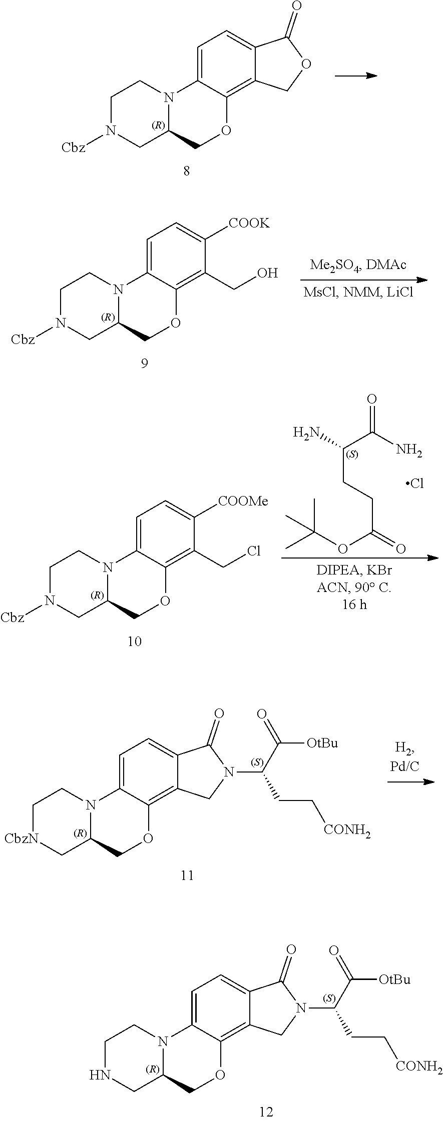

### Step 1

To a solution of 5-Bromo-3H- isobenzofuran-1-one (1) (50 g, 1 eq.) in trifluoromethanesulfonic acid (400 mL, 20 eq.) was added NIS (62.5 g, 1,2 eq.) at 0° C. in portions. The mixture was allowed to warm to room temperature and stirred overnight. TLC showed no starting material remained and two new spots formed. The reaction mixture was poured into iee-water and yellow solid precipitated. The mixture was filtered and the filter cake was washed with ice cold water. The filter cake was dissolved in DCM and washed with 1 (M) Na₂S203 followed by dried over sodium sulfate. The mixture was filtered and the filtrate was concentrated to afford a yellow solid. The crude product was purified by silica gel flash chromatography. The less polar product (top spot on TLC)₂ was obtained as a brown solid (40 g, yield=50%), and the more polar product 2b (bottom spot on TLC) was obtained as a brown solid which was not further reacted in next step.

### Step 2

To a mixture of compound 2 (20 g, 1 eq.), sodium hydroxide (11.5 g, 5 eq.) in water (200 mL, 1.5 M) and N,N-dimethylacetamide (100 mL) was added cuprous oxide (1.7 g, 0.2 eq.). The reaction mixture was heated to 80° C. and stirred for 12 h. TLC showed the reaction was completed. The reaction mixture neutralized using 1 (N) hydrochloride solution and extracted with ethyl acetate, washed with brine, and then dried over sodium sulfate. The crude product was purified by silica gel column chromatography to give compound 3 was obtained as a white solid (6.88 g, 51% yield).

### Step 3

To a solution of compound 3 (7 g, 1 eq.) in 120 ml of THF/DCM (V/V=2:1), compound 4 (20 g, 1.5 eq.) and PPh₃ (12 g, 1.55 eq.) was added. The reaction mixture was cooled to 0° C. and DIAD (9.5 mL, 1.55 eq.) was added dropwise. The resultant mixture was then stirred overnight at room temperature. The solvent was evaporated at reduced pressure and the crude product was purified by silica gel column chromatography using 0-100% EtOAc/hexane. The desired product 5 was obtained as a yellow foam (7 g, yield=35%).

### Step 4

To a solution of compound 5 (5 g, 1 eq.) in DMF (0.2 M) was added DBU (2 g). After TLC showed the reaction was completed, the mixture was diluted with ethyl acetate and washed with water. The combined organic phases were washed with brine, dried over Na₂SO₄ and the solvent was removed in vacuo to give an oil. The crude product was purified by silica gel column chromatography using 0-5% DCM in methanol to give compound 6 (2.8 g yield 85%). LC/MS (ESI) m/z: 426.08 [M+H]⁺.

### Step 5

A vial was charged with compound 6 (2.8 g, 0.38 mmol, 1 eq.), Pd₂ (dba) 3 (0.15 eq.), XantPhos (0.3 eq.), Cs₂CO₃ (5 eq.) and dioxane (100 mL). The mixture was purged with nitrogen and heated to 100° C. for 6 h. TLC showed reaction was complete. The mixture was diluted with ethyl acetate and washed with water. The organic layer was washed with brine and dried over sodium sulfate. The crude product was purified by silica gel column chromatography using 0-50% EtOAc/hexane to give compound 7 as a yellow foam (1,25 g, yield 55%), LC/MS (ESI) m/z: 347.15 [M+H]⁺.

### Step 6

To a stirred solution of compound 7 (2.55 g, 7.4 mmol, 1 eq.) in DCM (10 mL) at room temperature was added TFA (10 mL) slowly and the reaction mixture was stirred at rt for 1 hour. Once the reaction was completed, the mixture was concentrated and the residue was used directly for next step. The residue was diluted with DCM (50 mL), added TEA (2,3 g, 22.1 mmol, 3 eq.) and cooled to 0° C. was added CbzCl (1.8 g, 11,1 mol, 1.5 eq.) and the mixture was warmed to 25° C. and stirred for 3 h. Then, water and DCM were added and the organic phase was separated, washed with brine, dried and concentrated. The residue was further purified by silica column chromatography eluting with 30% EtOAc in PE to give compound 8 (2.5 g, 89%) as a light yellow solid.

### Step 7

A slurry of the compound 8 (3.1 g, 8.16 mmol) in i-PrOH (50 mL) and water (0.5 mL) was stirred in a reactor at ambient temperature. Potassium hydroxide (502 mg, 8.97 mmol) was charged as a solid. The resulting slurry was heated to T=35-40° C. and held at that temperature for 2 h. The slurry was cooled to 20-25° C., and then was filtered. The cake was washed with a mixture of i-PrOH and water. The cake (9) was dried at 25° C. under vacuum which was used directly for next step.

### Step 8

The compound 9 (3.56 g, 8.16 mmol) was slurried in DMAc (50 mL) at 10° C. in a three necks flask. Me₂SO₄ (1.08 g, 8.57 mmol, 1.05 equiv.) was added dropwise at 10° C. The resulting mixture was stirred at this temperature for 3 h. NMM (2.47 g, 24.5 mmol) was then added to the mixture, followed by the slow addition of MsCl (1.86 g, 16.3 mmol), maintaining 10-15° C. The resulting mixture was stirred at this temperature for 30 minutes. LiCl (514 mg, 12.2 mmol) was added as a solid in a single portion. The resulting slurry was heated to 35-40° C. for 1-2 h. The slurry was cooled to 20-25° C. and quenched with H₂O (200 mL). The mixture extracted with EtOAc (5×50 mL) and the organic layer dried over Na₂SO₄, filtered, and evaporated to afford the crude product, which was purified by flash silica chromatography to afford the compound 10 (2.1 g, two steps, 60%).

### Step 9

To a 100 mL reactor was charged 10 (900 mg, 2.09 mmol), tert-Butyl(S)-4,5-diamino-5-oxopentanoate hydrochloride (748 mg, 3.13 mmol), and potassium bromide (372 mg, 3.13 mmol), followed by acetonitrile (20 mL), and diisopropylethylamine (808 mg, 6.26 mmol). The resulting mixture was stirred at 90° C. for 16 h. The reaction was then cooled to 20° C. and diluted with water. The aqueous layer was extracted with EtOAc (3×50 mL). The combined organic phase was washed sequentially with water (20 mL) and saturated brine (20 mL). The organic layer was dried over Na₂SO₄, filtered, and evaporated to afford crude product. The crude product was purified by flash silica chromatography to yield 11 (500 mg) LC-MS: [M+H]⁺: 565.

Step 10:

A 2,2,2-Trifluoroethanol solution of the product 11 (500 mg) from Step 9 was added palladium on activated carbon catalyst (0.15 eq., 10% purity) under nitrogen. The suspension was degassed under vacuum and purged with hydrogen three times. The mixture was heated at 25° C. for 3 h, then filtered and concentrated in vacuo. The residue was used directly for next step. LC-MS: [M+H]⁺: 431.

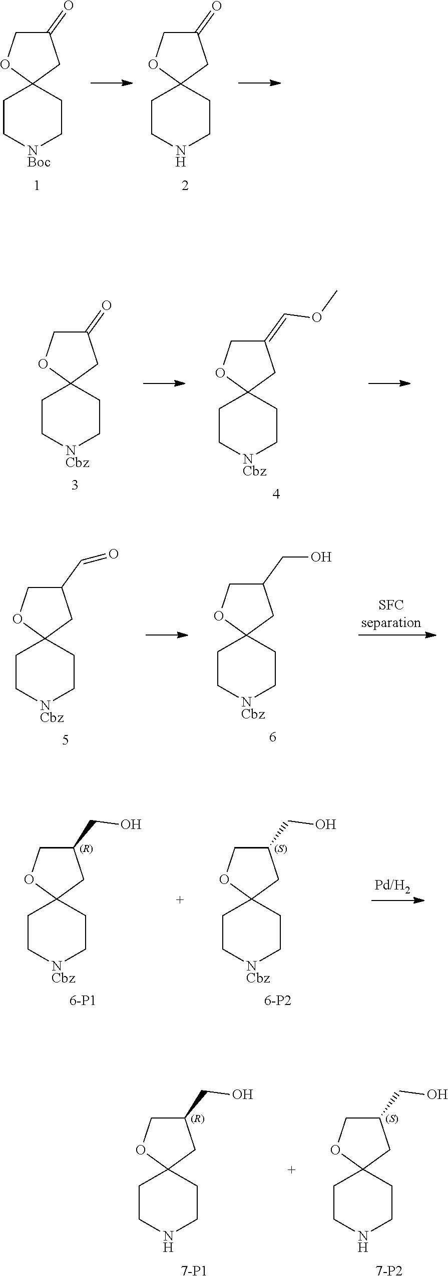

### Step 1-2

To a stirred solution of compound 1 (5 g, 0.0196 mol, 1 eq.) in EtOAc (50 mL) at room temperature was added conc. HCl (10 g, 0.098 mol, 5 eq.) slowly and the reaction mixture was stirred at rt for 1 hour. Once the reaction was completed, the mixture was diluted with EtOAc (50 mL), poured into Na₂CO₃ suspension (20.8 g, 0.196 mol, 10 eq., in 100 ml of water) and the mixture was stirred for 20 min. To the mixture was added CbzOSu (4.9 g, 0.0196 mmol, 1 eq.) and the mixture was stirred for 1 h. The organic phase was separated, washed with brine, dried, concentrated and the residue was purified by silica column chromatography eluting with 50% EtOAc in PE to give compound 3 (5,6 g, 100%) as a light yellow oil.

### Step 3

To a stirred solution of (methoxymethyl)triphenylphosphonium chloride (16.6 g, 0.071 mol, 2.5 eq.) in dried THF (100 mL) cooled at −70° C. was added NaHMDS (19.4 mL, 0.0470 mol, 2 eq.) dropwise and the mixture was warmed to 0° C. slowly and stirred for 2 h. Then the mixture was cooled at −70° C. and a solution of compound 3 (5,6 g, 0.1 mol, 1 eq.) in THF (50 mL) was added. The mixture was warmed to rt slowly and stirred for 2 h. TLC was done to detect the process of the reaction. Once no starting material was left, the mixture was quenched by NH₄Cl solution (150 mL) and diluted with EtOAc (200 mL). The organic phase was separated, washed with brine, dried, concentrated and the residue was purified by silica column chromatography eluting with 30% EtOAc in PE to give compound 4 (5.8 g, 94%) as a light yellow oil.

### Step 4

A solution of compound 4 (5.8 g, 0.0183 mol, 1 eq.) in ACN (80 mL) and 2M HCl (40 mL) was stirred at rt for 4 hours. TLC were done to detect the process of the reaction. Once the reaction was completed, the mixture was quenched by water (100 mL) and extracted with EtOAc (100 mL*3). The organic phase was separated, washed with brine, dried, concentrated and the residue was purified by silica column chromatography eluting with 30% EtOAc in PE to give compound 5 (4.6 g, 0.0153 mol, 78%) as a light yellow oil.

### Step 5

To a stirred solution of compound 5 (4.6 g, 0.0153 mol, 1.0 eq) in MeOH (100 mL) cooled at 0° C. was added NaBH₄ (0.88 g, 0.0231 mol, 1.5 eq.) and the mixture was warmed to 0˜25° C. and stirred for 1 h. TLCs were done to detect the process of the reaction. Once the reaction was completed, the mixture was quenched by water (100 mL) and extracted with EtOAc (100 mL*3). The organic phase was separated, dried, concentrated and the residue was purified by silica column chromatography eluting with 50% EtOAc in PE to give compound 6 (4.5 g, 0.015 mol, 98%) as a light yellow oil, ¹H NMR (500 MHz, Chloroform-d) δ 7.38-7.27 (m, 5H), 5.12 (s, 2H), 3.95 (dd, J=9.0, 7.2 Hz, 1H), 3.76-3.55 (m, 5H), 3.36 (m, 2H), 2.61-2.48 (m, 1H), 1.90 (dd, J=12.7, 8.6 Hz, 1H), 1.81-1.45 (m, 4H), 1.42 (dd, J=12.7. 7.6 Hz, 1H).

### Step 6

The compound 6 was further separated by preparative SFC(Column: ChiralPak AD, 250×30 mm I.D., 10 μm, Mobile phase: A for CO₂ and B for Ethanol, Gradient: B 40%, Flow rate: 140 mL/min, Back pressure: 100 bar) to afford a pure product 6-P1 (2 g) and a pure product 6-P2 (2 g).

### Step 7

To a solution of compound 6-P1 (0.78 g, 1 eq.) in CF₃CH₂OH (15 mL) was added Pd/C (0.3 g, 10% on Carbon, wetted with ca, 55% water) and the mixture was stirred at rt for 12 hours under H₂ (balloon). TLC were done to detect the process of the reaction. Once the reaction was completed, the catalyst was removed by filtration and the filtrate was concentrated to give compound 7-P1 (0.43 g, 100%).

According to the same procedure, the title compound 7-P2 was provided as a colorless oil.

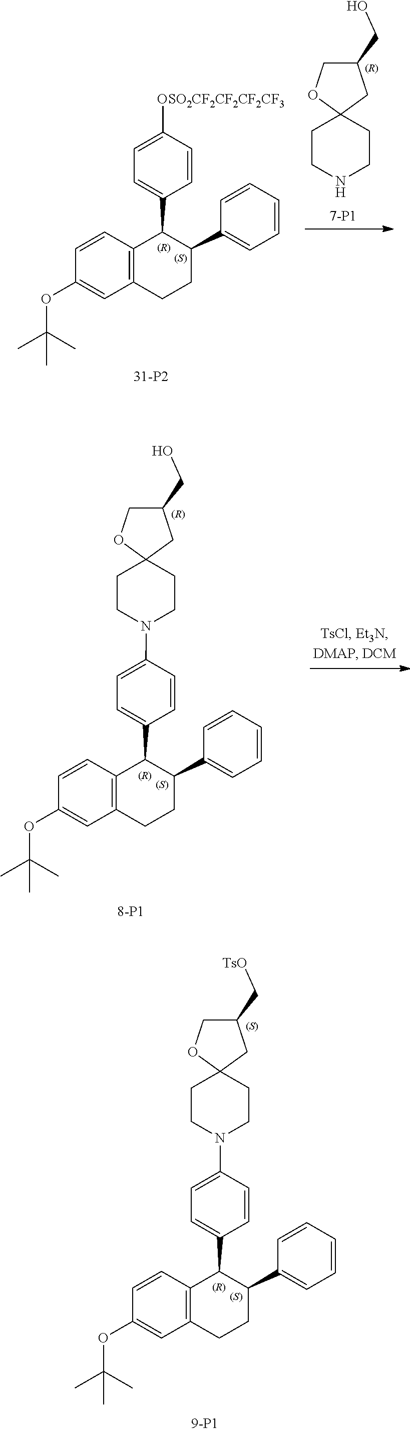

### STEP 1

A round bottomed flask equipped with a stirrer bar was charged with a mixture of compound 31-P2 (650 mg, 1 mmol), chiral amine 7-P1 (205 mg, 1,2 mmol), cesium carbonate (650 mg, 2 mmol), and t-BuXphoxPd-G (III) (CAS: 1447963-75-8, 80 mg, 0.1 mmol). The flask was evacuated and back-filled with nitrogen (x 3), t-Amyl alcohol (10 mL) was added and the mixture stirred at 100° C. for 10 hours. The cooled reaction mixture was diluted with EtOAc and filtered through Celite™ to remove insoluble material. The filtrate was washed with water, saturated aqueous sodium chloride and then dried over magnesium sulfate, filtered and the filtrate concentrated. The crude material was purified by flash silica chromatography, elution gradient MeOH in DCM. Pure fractions were combined and concentrated to afford compound 8-P1 (180 mg, 35%). LC-MS: [M+H]⁺: 526; 1H NMR (400 MHZ, Chloroform-d) 8 7.19-7.10 (m, 3H), 6.88 -6.77 (m, 4H), 6.72 (dd, J=8.3, 2.4 Hz, 1H), 6.56 (d, J=8.4 Hz, 2H), 6.25 (d, J=8.4 Hz, 2H), 4.22 (d, J=5.0 Hz, 1H), 3.97 (dd, J=9.0, 7.2 Hz, 1H), 3.65 (m, 5H), 3.36 (m, 1H), 3.10 (m, 5H), 2.55 (m, 1H), 2.24-2.11 (m, 2H), 1.93 (m, 1H), 1.76-1.62 (m, 5H), 1.36 (s, 9H),

### STEP 2

To a solution of compound 8-P1 (180 mg, 0.34 mmol, 1 eq.) in DCM (5 mL) was added TEA (0.68 mmol, 2 eq.) and DMAP (0.04 mmol, 0.2 eq.). The flask was evacuated and back-filled with nitrogen. TsCl (0.51 mmol, 1.5 eq.) was added and the mixture stirred at R.T, overnight. TLC indicated the starting material was consumed completely. The reaction mixture was quenched by addition of saturated NH₄Cl solution, and extracted with ethyl acetate. The combined organic layers were washed with brine, dried over sodium sulfate, filtered and concentrated under reduced pressure. The crude material was purified by flash silica chromatography to give compound 9-P1 (150 mg, 65%). LC-MS [M+H]⁺: 680; 1H NMR (600 MHz, Chloroform-d) δ 7.78 (d, J=8.0 Hz, 2H), 7.35 (d, J=8.0 Hz, 2H), 7.14 (m, 3H), 6.88-6.82 (m, 2H), 6.81-6.77 (m, 2H), 6.72 (dd, J=8.4, 2.4 Hz, 1H), 6.54 (d, J=8.3 Hz, 2H), 6.26 (d, J=8.3 Hz, 2H), 4.22 (d, J=5.0 Hz, 1H), 4.04-3.93 (m, 2H), 3.89 (m, 1H), 3.54 (dd, J=9.3, 6.1 Hz, 1H), 3.36 (ddd, J=13.2, 5.4, 2.2 Hz, 1H), 3.05 (m, 6H), 2.71-2.60 (m, 1H), 2.45 (s, 3H), 2.17 (m, 1H), 1.89 (dd, J=12.9, 8.7 Hz, 1H), 1.79 (m, 1H), 1.74-1.66 (m, 3H), 1.60 (m, 2H), 1.36 (s, 9H),

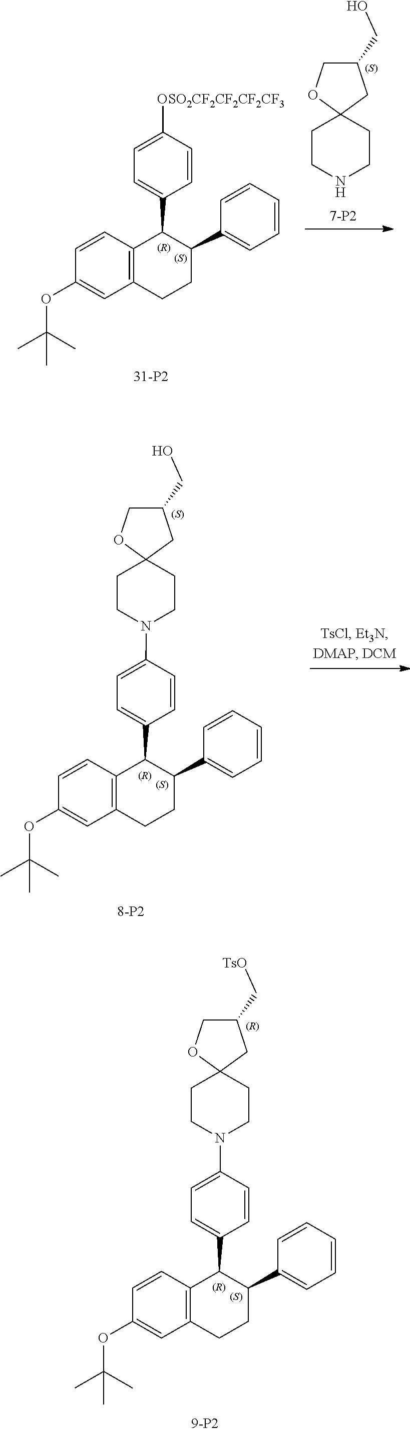

According to the same procedure, chiral amine 7-P2 was applied to afford compound 9-P2, LC-MS: [M+H]⁺: 680; 1H NMR (400 MHZ, Chloroform-d) 8 7.78 (d, J=8.3 Hz, 2H), 7.35 (d, J=8.3 Hz, 2H), 7.14 (m, 3H), 6.88-6.82 (m, 2H), 6.80 (dd, J=7.3, 2.2 Hz, 2H), 6.72 (dd, J=8.3, 2.5 Hz, 1H), 6.54 (d, J=8.6 Hz, 2H), 6.24 (d, J=8.6 Hz, 2H), 4.22 (d, J=4.9 Hz, 1H), 4.02-3.82 (m, 3H), 3.54 (dd, J=9.3, 6.1 Hz, 1H), 3.41-3,29 (m, 1H), 3.05 (m, 6H), 2.66 (m, 1H), 2.45 (s, 3H), 2.16 (m, 1H), 1.89 (m, 1H), 1.78 (m, 1H), 1.36 (s, 9H).

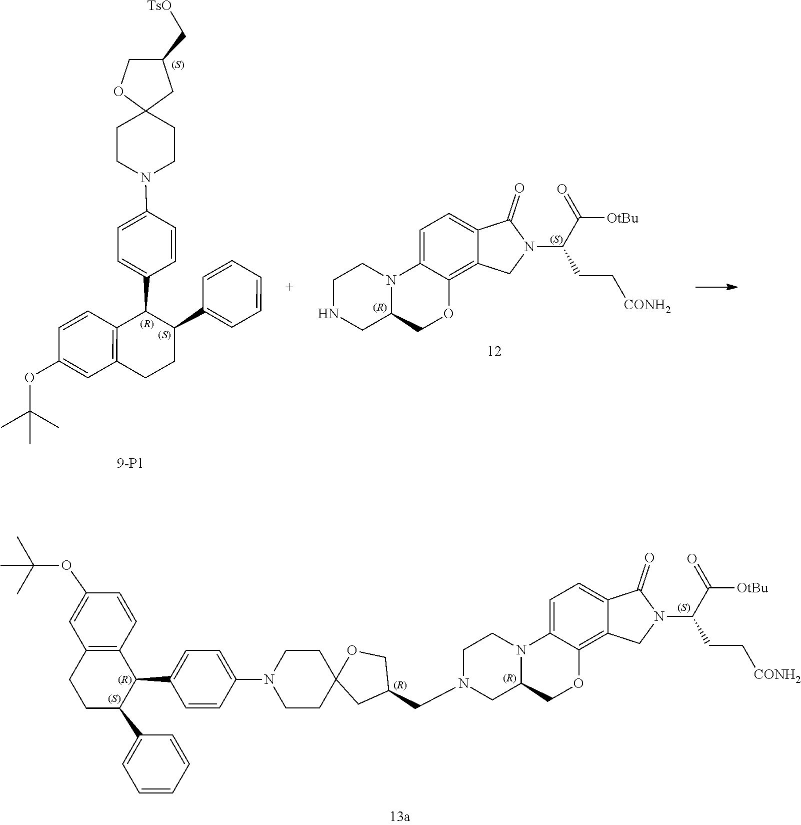

To a solution of compound 9-P1 (160 mg, 0.235 mmol, 1 eq.) in acetonitrile (2 mL) was added 12 (111 mg, 0.258 mmol, 1,1 eq.), KI (40 mg, 0.235 mmol, 1.0 eq.) and diisopropylethylamine (0.12 mL, 3 eq.). The mixture was stirred at 90° C. for 24 hours. LC-MS showed the reaction was completed. The mixture was diluted with water and extracted with ethyl acetate. The combined organic layers was washed wash brine, dried with anhydrous sodium sulfate, filtered and the filtrate was concentrated in vacuum. The residue was purified was purified by flash silica chromatography (DCM:MeOH=10:1) to give the title compound 13a (160 mg, 72%), LC-MS: [M+H]⁺: 938.5.

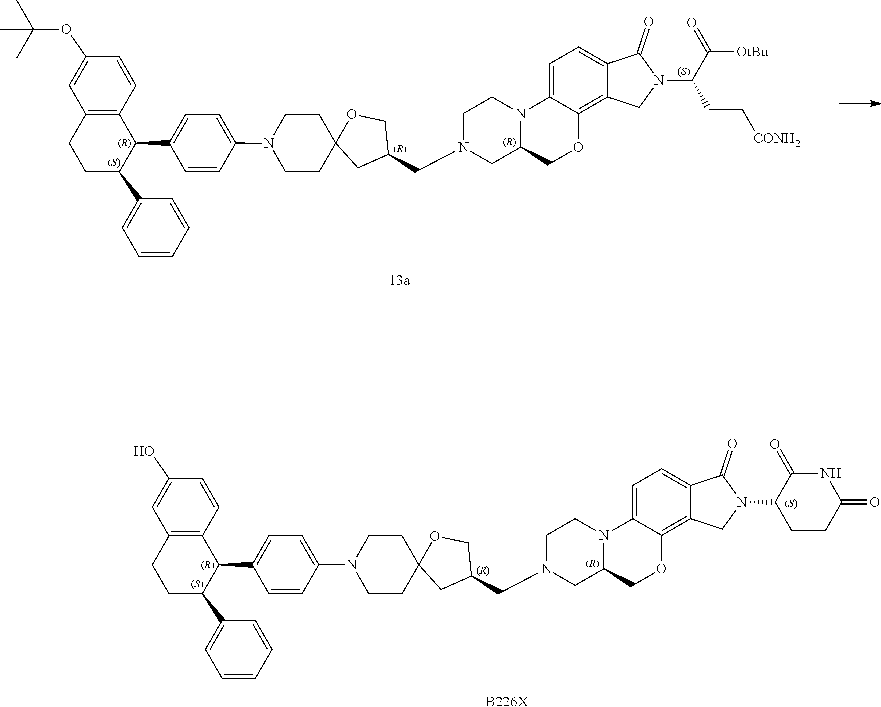

To a solution of compound 13a (100 mg, 0.11 mmol, 1 eq.) in acetonitrile (3 mL) was added benzenesulfonic acid (70 mg, 0.44 mmol, 4 eq.). The mixture was stirred at 80° C. for 3 hours. LC-MS showed the reaction was completed. The mixture was concentrated in vacuum. The residue was purified by preparative reverse phase HPLC(ACN:H₂O=25-55% (1% 0 TFA) to provide the title compound B226X (53 mg, 60%). LC-MS: [M+H]⁺: 808.4.

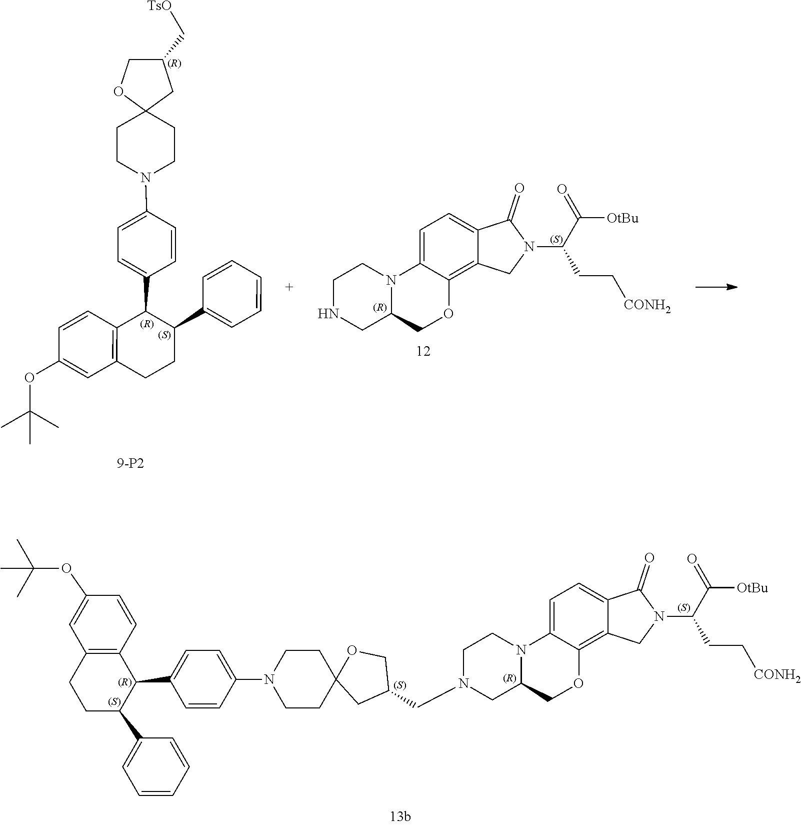

To a solution of compound 9-P2 (160 mg, 0.235 mmol, 1 eq.) in acetonitrile (2 mL) was added 12 (111 mg, 0.258 mmol, 1,1 eq.), KI (40 mg, 0.235 mmol, 1.0 eq.) and diisopropylethylamine (0.12 mL, 3 eq.). The mixture was stirred at 90° C. for 24 hours. LC-MS showed the reaction was completed. The mixture was diluted with water and extracted with ethyl acetate. The combined organic layers were washed wash brine, dried with anhydrous sodium sulfate, filtered and the filtrate was concentrated in vacuum. The residue was purified was purified by flash silica chromatography (DCM:MeOH=10:1) to give the title compound 13b as a white solid. LC-MS: [M+H]⁺: 938.5.

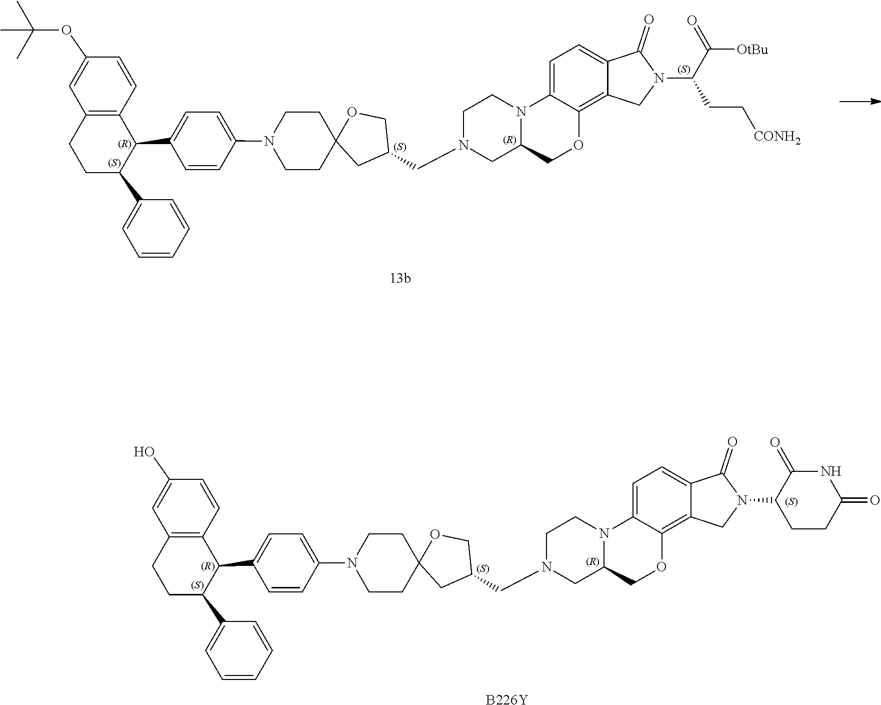

To a solution of compound 13b (100 mg, 0.11 mmol, 1 eq.) in acetonitrile (3 mL) was added benzenesulfonic acid (70 mg, 0.44 mmol, 4 eq.). The mixture was stirred at 80° C. for 3 hours. LC-MS showed the reaction was completed. The mixture was concentrated in vacuum. The residue was purified by preparative reverse phase HPLC(ACN:H₂O=25-55% (1% c TFA) to provide the title compound B226Y as a white solid. LC-MS: [M+H]⁺: 808.4.

### Compound 227X. (S)-3-((S)-3-(((S)-8-(4-((1R,2S)-6-hydroxy-2-phenyl-1,2,3,4-tetrahydronaphthalen-1-yl)phenyl)-1-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

### Compound 227Y. (S)-3-((S)-3-(((R)-8-(4-((1R,2S)-6-hydroxy-2-phenyl-1,2,3,4-tetrahydronaphthalen-1-yl)phenyl)-1-oxa-8-azaspiro[4.5]decan-3-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

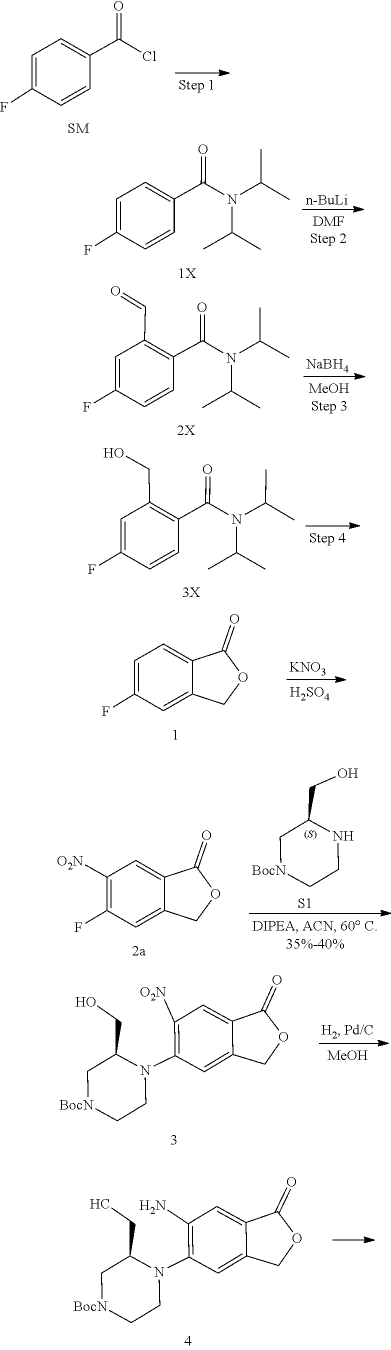

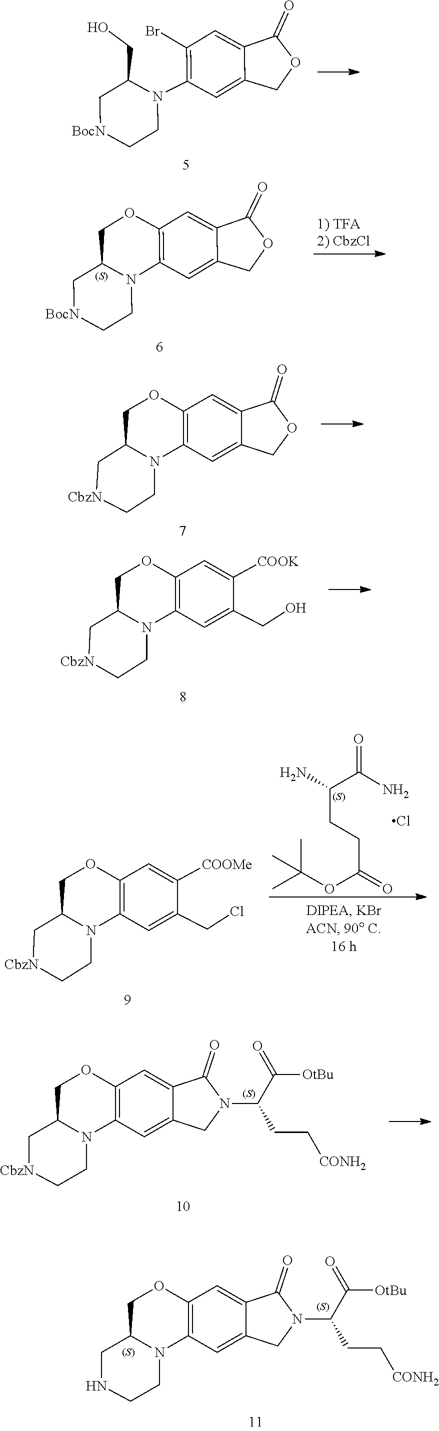

### Step 1

A mixture of diisopropylamine (382.9 g, 3.78 mol, 1,2 eq.) and K₂CO₃ (522.9 g, 3.78 mol, 1,2 eq.) in toluene (1500 mL) and water (1500 mL) was added to a solution of SM (500 g, 3.15 mol, 1.0 eq.) at 5-15° C. After stirring for 3 h at ambient temperature, the aqueous layer was extracted with ethyl acetate. Then, the organic layer was further washed with 1N HCl, and brine, dried over MgSO₄, and evaporated. The residue was triturated with hexane (200 mL), and the crystalline product was collected by filtration to give 1X (500 g, 71%) as colorless crystals.

### Step 2

n-Butyllithium (948 mL of a 2.5 M solution in hexane, 2.37 mol, 1,1 eq.) was added to a solution of 1X (500 g, 2.24 mol, 1.0 eq.) in dry THF (3000 mL) at −78° C. After stirring for 1 h at −78° C. DMF (173 g, 2.37 mol, 1,1 eq.) was added, while the temperature of the mixture rose to −70° C. After warming to ambient temperature, the reaction mixture was diluted with a saturated aqueous solution of ammonium chloride (2000 mL) and extracted with ethyl acetate (2000 mL). The organic layer was washed with brine and dried over MgSO₄. The solvent was removed in vacuum to give 2X as a white solid (483 g, 85% yield).

### Step 3

Sodium borohydride (72.8 g, 1.0 eq.) was added in portion to a solution of 2X (483 g, 1.0 eq.) in dry methanol (3000 mL) and cooled with ice-water bath. After stirring for 10 h, the solvent was evaporated, water (1000 mL) was added to the residue, and the mixture was extracted with diethyl ether (2000 mL). The ethereal layer was washed with brine, dried over MgSO₄. The solvent was removed in vacuum to give 3X as a beige solid (450 g, 93% yield).

### Step 4

A mixture of 3X (450 g) and aqueous hydrochloric acid (6 M, 2250 mL) was refluxed for 10 h. After cooling to ambient temperature it was extracted with ethyl acetate (3×1500 mL). The combined organic layer was extracted with sat. NaHCO₃ (1500 Ml) and then washed with brine, dried over MgSO₄, and evaporated. The residue was triturated with a mixture (20:1) of PE and ethyl acetate (300 mL) and collected by filtration to give 1 (52%) Step 5

To a solution of 1 (30 g, 1.0 equiv) in H₂SO₄ (150 mL) was added KNO₃ (1.5 equiv, 30 g) in potionwise. The reaction mixture was stirred at rt for 3 h. TLC (PE:EA=2:1) indicated the starting material was consumed completely and two new spot formed. The reaction mixture was slowly poured into ice water, then extracted with EA, washed with brine, dried (Na₂SO₄) and concentrated under reduced pressure to give a residue which was purified by silica gel flash chromatography. The minor product 2b (the less polar product) was obtained as a white solid (8 g, yield=20%), and the major product 2a (the more polar product) was obtained as a white solid (30 g, yield=80%).

### Step 6

To a solution of 2a (1.0 equiv, 10 g) and S1 (1,2 equiv, 13.2 g) in acetonitrile (100 mL) was added DIPEA (2.5 equiv, 22 mL), and the reaction mixture was stirred at 60° C. for 6 h. TLC (DCM:MeOH=20:1) showed the starting material 2a was completely consumed. Then the reaction mixture was concentrated under reduced pressure and the result residue was purified by silica gel flash chromatography. The desired product 3 was obtained as a yellow foam (7 g, yield=35%).

### Step 7

To a solution of 3 (10 g, 1.0 equiv) in MeOH (100 mL) was added Pd/C(2 g, 20% w). The reaction mixture was degassed and purged with H₂ three times and keep stirred at rt overnight. UPLC-MS showed the starting material completely conversed to desired product 4. Then the reaction mixture was filtered through Celite, and the filtrate was concentrated under reduced pressure to give a residue which was purified by silica gel flash chromatography. The desired product 4 was obtained as a yellow solid (8.3 g, yield=90%).

### Step 8

To a stirred solution of 4 (5 g, 1 eq.) in dry acetonitrile (100.0 mL) was added CuBr₂ (2.76 g, 0.9 eq) at 0° C. After 5 min, a solution of tert-butyl nitrite (1.9 mL, 1,2 eq) in dry acetonitrile (5.0 mL) was added and the reaction mixture was stirred for 0.5 h. TLC (DCM:MeOH=50:1, Rf=0.5). The mixture was diluted with ethyl acetate, washed with saturated sodium bisulfite, dried (Na₂SO₄), filtered and concentrated in vacuo. Purify the residue by silica gel column chromatography and elute with MeOH/DCM (0% to 2%). The desired product 5 was obtained as a yellow solid (3.2 g, yield=55%).

### Step 9

A vial was charged with 5 (3.6 g, 1 eq.), Pd(OAc)₂ (0.42 g, 0.2 eq.), JohnPhos (3.2 g, 1,2 eq.). Cs₂CO₃ (9 g, 3 eq.) and toluene (200 mL). The mixture was purged with nitrogen and heated to 90° C. for 6 h. TLC (ethyl acetate:petroleum ether=1:1) showed reaction was complete. The mixture was filtered and the filtrate was concentrated in vacuum. The crude product was triturated with Et₂O to afford the title compound 6 as a beige solid (1.75 g, 60% yield).

### Step 10

To a stirred solution of compound 6 (2.55 g, 7.4 mmol, 1 eq.) in DCM (10 mL) at room temperature was added TFA (10 mL) slowly and the reaction mixture was stirred at rt for 1 hour. Once the reaction was completed, the mixture was concentrated and the residue was used directly for next step. The residue was diluted with DCM (50 mL), added TEA (2,3 g, 22.1 mmol, 3 eq.) and cooled to 0° C. was added CbzCl (1.8 g, 11,1 mol, 1.5 eq.) and the mixture was warmed to 25° C. and stirred for 3 h. Then, water and DCM were added and the organic phase was separated, washed with brine, dried and concentrated. The residue was further purified by silica column chromatography eluting with 30% EtOAc in PE to give compound 7 (2.24 g, 80%) as a light yellow solid.

### Step 11

A slurry of the compound 7 (5 g, 8.16 mmol) in i-PrOH (50 mL) and water (0.5 mL) was stirred in a reactor at ambient temperature. Potassium hydroxide (502 mg, 8.97 mmol) was charged as a solid. The resulting slurry was heated to T=35-40° C. and held at that temperature for 2 h.

The slurry was cooled to 20-25° C., and then was filtered. The cake was washed with a mixture of i-PrOH and water. The cake (8) was dried at 25° C. under vacuum which was used directly for next step.

### Step 12

The compound 8 (3.56 g, 8.16 mmol) was slurried in DMAc (50 mL) at 10° C. in a three necks flask. Me₂SO₄ (1.08 g, 8.57 mmol, 1.05 equiv.) was added dropwise at 10° C. The resulting mixture was stirred at this temperature for 3 h. N-methylmorpholine (NMM) (2.47 g, 24.5 mmol) was then added to the mixture, followed by the slow addition of MsCl (1.86 g, 16.3 mmol), maintaining 10-15° C. The resulting mixture was stirred at this temperature for 30 minutes. LiCl (514 mg, 12.2 mmol) was added as a solid in a single portion. The resulting slurry was heated to 35-40° C. for 1-2 h. The slurry was cooled to 20-25° C. and quenched with H₂O (200 mL). The mixture extracted with EtOAc (5×50 mL) and the organic layer dried over Na₂SO₄, filtered, and evaporated to afford the crude product, which was purified by flash silica chromatography to afford the compound 9 (2.1 g, two steps, 60%)

### Step 13

To a 100 mL reactor was charged 9 (900 mg, 2.09 mmol), tert-Butyl(S)-4,5-diamino-5-oxopentanoate hydrochloride (748 mg, 3.13 mmol), and potassium bromide (372 mg, 3.13 mmol). followed by acetonitrile (20 mL), and diisopropylethylamine (808 mg, 6.26 mmol). The resulting mixture was stirred at 90° C. for 16 h. The reaction was then cooled to 20° C. and diluted with water. The aqueous layer was extracted with EtOAc (3×50 mL). The combined organic phase was washed sequentially with water (20 mL) and saturated brine (20 mL). The organic layer was dried over Na₂SO₄, filtered, and evaporated to afford crude product. The crude product was purified by flash silica chromatography to yield 10 (500 mg) LC-MS: [M+H]⁺: 565.4.

### Steps 14

A 2,2,2-Trifluoroethanol solution of the product 10 (500 mg) from Step 13 was added 10% palladium on activated carbon catalyst (0.15 eq.) under nitrogen. The suspension was degassed under vacuum and purged with hydrogen three times. The mixture was heated at 25° C. for 3 h, then filtered and concentrated in vacuo. The residue (11) was used directly for next step. LC-MS: [M+H]⁺: 431,2

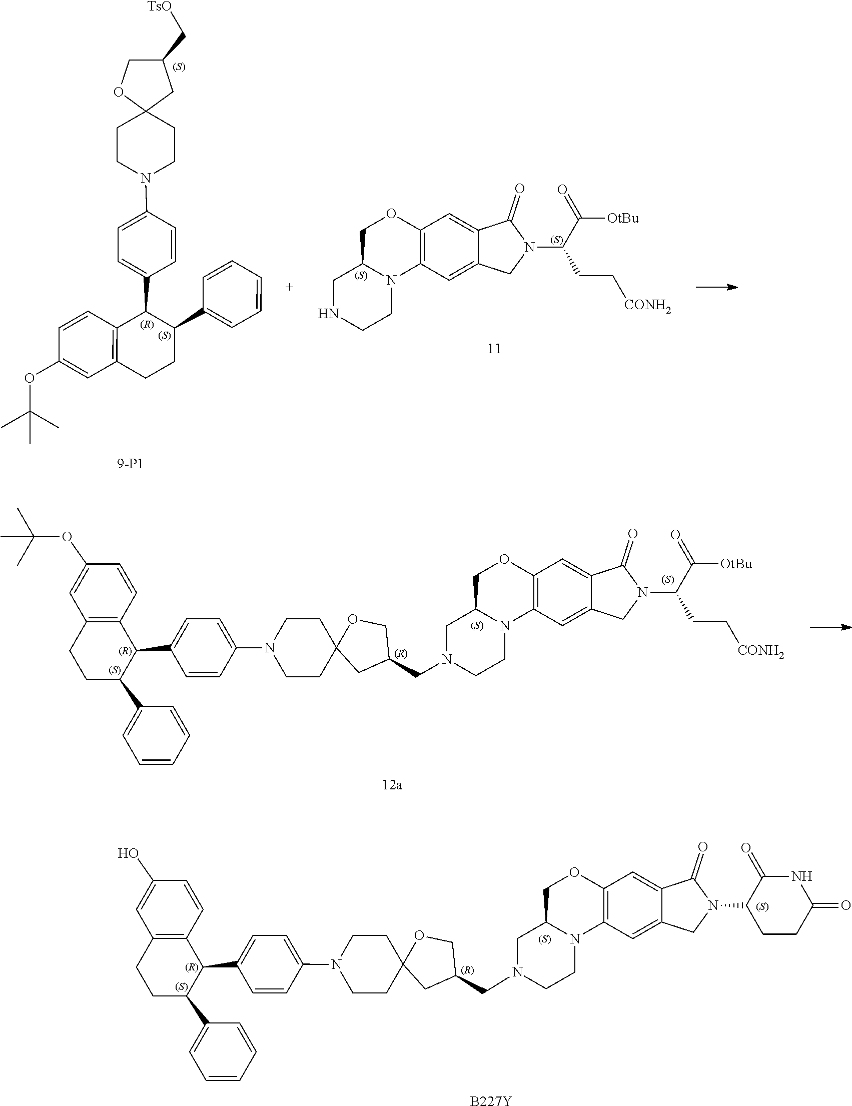

### Step 1

To a solution of compound 9-P1 (160 mg, 0.235 mmol, 1 eq.) in acetonitrile (2 mL) was added 11 (111 mg, 0.258 mmol, 1,1 eq.), KI (40 mg, 0.235 mmol, 1.0 eq.) and diisopropylethylamine (0.12 mL, 3 eq.). The mixture was stirred at 90° C. for 24 hours. LC-MS showed the reaction was completed. The mixture was diluted with water and extracted with ethyl acetate. The combined organic layers was washed wash brine, dried with anhydrous sodium sulfate, filtered and the filtrate was concentrated in vacuum. The residue was purified was purified by flash silica chromatography (DCM:MeOH=10:1) to give the title compound 12a (155 mg, 70%), LC-MS: [M+H]⁺: 938.5.

### Step 2

To a solution of compound 12a (100 mg, 0.11 mmol, 1 eq.) in acetonitrile (3 mL) was added benzenesulfonic acid (70 mg, 0.44 mmol, 4 eq.). The mixture was stirred at 80° C. for 3 hours. LC-MS showed the reaction was completed. The mixture was concentrated in vacuum. The residue was purified by preparative reverse phase HPLC(ACN:H₂O=25-55% (1% 0 TFA) to provide the title compound B227Y (53 mg, 60%). LC-MS: [M+H]⁺: 808.4.

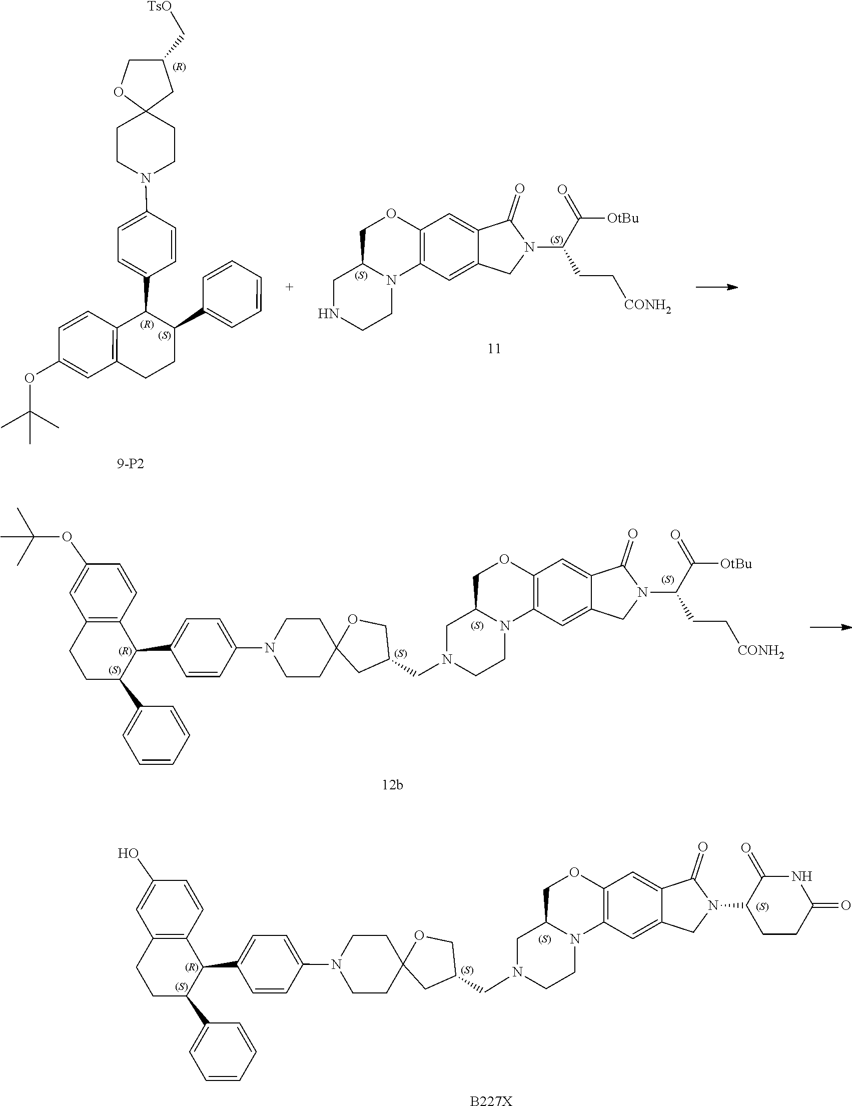

### Step 1

To a solution of compound 9-P2 (160 mg, 0.235 mmol, 1 eq.) in acetonitrile (2 mL) was added 11 (111 mg, 0.258 mmol, 1,1 eq.), KI (40 mg, 0.235 mmol, 1.0 eq.) and diisopropylethylamine (0.12 mL, 3 eq.). The mixture was stirred at 90° C. for 24 hours. LC-MS showed the reaction was completed. The mixture was diluted with water and extracted with ethyl acetate. The combined organic layers was washed wash brine, dried with anhydrous sodium sulfate, filtered and the filtrate was concentrated in vacuum. The residue was purified was purified by flash silica chromatography (DCM:MeOH=10:1) to give the title compound 12b. LC-MS: [M+H]⁺: 938.5.

### Step 2

To a solution of compound 12b (100 mg, 0.11 mmol, 1 eq.) in acetonitrile (3 mL) was added benzenesulfonic acid (70 mg, 0.44 mmol, 4 eq.). The mixture was stirred at 80° C. for 3 hours. LC-MS showed the reaction was completed. The mixture was concentrated in vacuum. The residue was purified by preparative reverse phase HPLC(ACN:H₂O=25-55% (1%. TFA) to provide the title compound B227X. LC-MS: [M+H]⁺: 808.4.

### Compound B240. (S)-3-((R)-7-((9-(4-((1R,2S)-6-hydroxy-2-phenyl-1,2,3,4-tetrahydronaphthalen-1-yl)phenyl)-1,5-dioxa-9-azaspiro[5.5]undecan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

### Compound B337. (S)-3-(1′-((9-(4-((1R,2S)-6-hydroxy-2-phenyl-1,2,3,4-tetrahydronaphthalen-1-yl)phenyl)-1,5-dioxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione

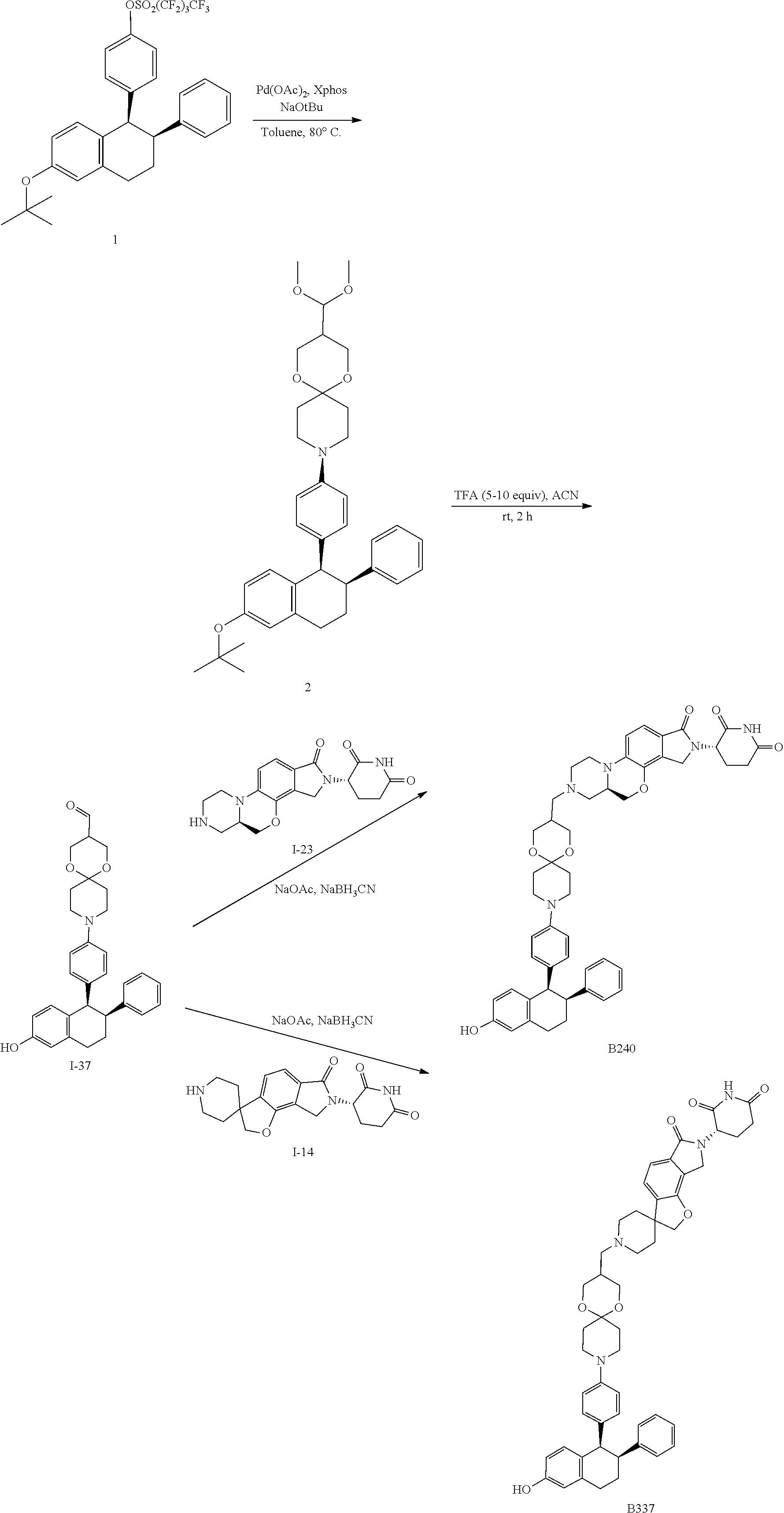

### Step 1:9-(4-((1R,2S)-6-(tert-butoxy)-2-phenyl-1,2,3,4-tetrahydronaphthalen-1-yl)phenyl)-3-(dimethoxymethyl)-1,5-dioxa-9-azaspiro[5.5]undecane

To a mixture of 4-((1R,2S)-6-(tert-butoxy)-2-phenyl-1,2,3,4-tetrahydronaphthalen-1-yl) phenyl 1,1,2,2,3,3,4,4,4-nonafluorobutane-1-sulfonate (100 mg, 0.15 mmol, 1.0 eq), 3-(dimethoxymethyl)-1,5-dioxa-9-azaspiro[5.5]undecane (42 mg, 0.18 mmol, 1,2 eq), t-BuONa (44 mg, 0.46 mmol, 3.0 eq) and Xphos (7 mg, 0.02 mmol, 0.1 eq) in toluene (5 mL) was added Pd(OAc)₂ (13 mg, 0.02 mmol, 0.1 eq), then stirred at 120° C. for 2 hours. LC-MS showed the reaction was completed. The reaction mixture was cooled to rt, the mixture was diluted with water and washed with EtOAc, the organic phase was dried with Na₂SO₄ and concentrated under vacuum, the residue was purified by SiO₂ column chromatography (EtOAc:PE=1:20) to afford compound 2 (71 mg, 80%) as a yellow oil. LC-MS purity: 62.1% (UV at 254 nm), LC-MS: 586.3 [M+H]⁺.

### Step 2:8-(4-((1R,2S)-6-hydroxy-2-phenyl-1,2,3,4-tetrahydronaphthalen-1-yl)phenyl)-1-oxa-8-azaspiro[4.5]decane-3-carbaldehyde

To a mixture of 7-(4-((1R,2S)-6-(tert-butoxy)-2-phenyl-1,2,3,4-tetrahydronaphthalen-1-yl)phenyl)-2-(dimethoxymethyl)-7-azaspiro[3.5]nonane (71 mg, 0.119 mmol, 1 eq) in HCOOH (3 mL) was stirred at 25° C. for 0.5 hour. The mixture was concentrated to give crude product 7-(4-((1R,2S)-6-hydroxy-2-phenyl-1,2,3,4-tetrahydronaphthalen-1-yl)phenyl)-7-azaspiro[3.5]nonane-2-carbaldehyde (49 mg, 85%) as a colorless liquid. LC-MS purity: 100% (UV at 254 nm), LC-MS: 484.3 [M+H]⁺.

### Step 3&4: (S)-3-((R)-7-((9-(4-((1R,2S)-6-hydroxy-2-phenyl-1,2,3,4-tetrahydronaphthalen-1-yl)phenyl)-1,5-dioxa-9-azaspiro[5.5]undecan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

To a solution of I-23 (1,2 equiv) in Methanol:DCM (4:1) was added NaOAc (2 equiv) and the mixture was stirred at room temperature for 5 mins. The aldehyde I-37 (1 equiv) in MeOH:DCM (4:1) was then added to the reaction mixture and stirred for another 10 mins. Sodiumcyanoborohydride (2 equiv) and Acetic acis was then added to the reaction sequentially and stirred for another 15 minutes. UPLC chromatography showed the completion of the reaction, and the product was purified using preparative HPLC. LC/MS (ESI) m/z: 824,4 (M+H).

(S)-3-(1′-((9-(4-((1R,2S)-6-hydroxy-2-phenyl-1,2,3,4-tetrahydronaphthalen-1-yl)phenyl)-1,5-dioxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione: LC/MS (ESI) m/z: 823,4 (M+H).

Compound B366X. (S)-3-(1′-(((S)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-Compound B366Y. (S)-3-(1′-(((R)-9-(4-((1R,2S)-6-hydroxy-2-phenyl-1,2,3,4-tetrahydronaphthalen-1-yl)phenyl)-1-oxa-9-azaspiro[5.5]undecan-3-yl)methyl)-6-oxo-6,8-dihydro-2H,7H-spiro[furo[2,3-e]isoindole-3,4′-piperidin]-7-yl)piperidine-2,6-dione

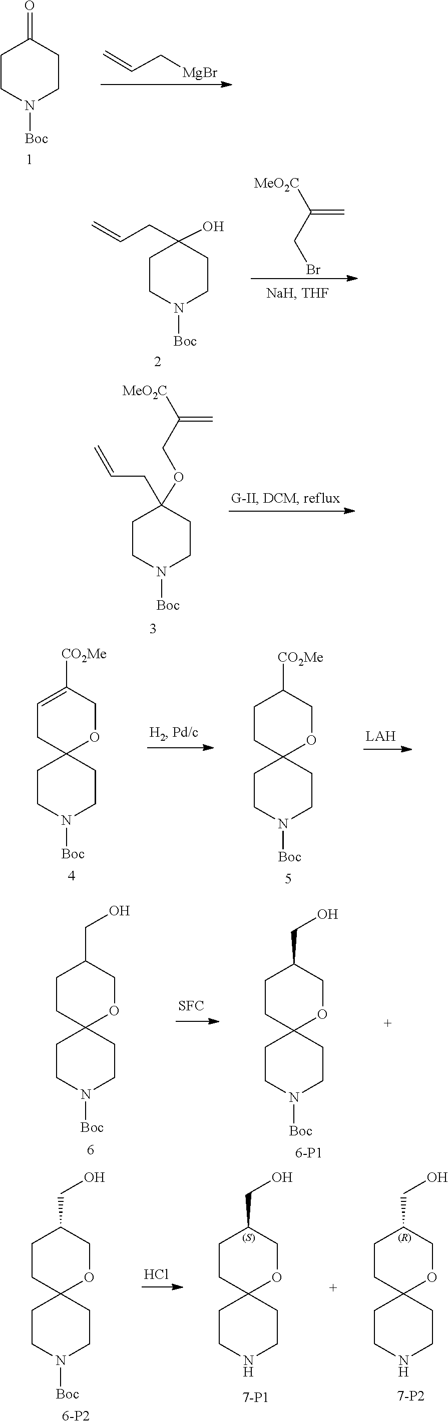

### Step 1

Add allylmagnesium bromide (15.2 g, 2.5 eq) to a solution of N-Boc-4-piperidone (1, 10.0 g, 50 mmol) in THF (40 mL). Then added sat, aq. NH₄Cl (40 mL), after that the Zn powder (6.6 g, 2.0 eq.) were added at r.t by three batch, then warm to 40° C. and stirred for 16 h. The mixture was diluted in water (200 mL) and extracted by EtOAc (100 mL*3). The organic layers was washed with water and brine, dried (Na₂SO₄) and concentrated. Purify the crude by flash chromatography (20-30% EtOAc/CyHex) to obtain the compound 2 (10.5 g), ¹H NMR: (400 MHZ, CDCl₃) δ 5.94-5.77 (m, 1H), 5.19 (dd, J=10.4, 1.8 Hz, 1H), 5.14 (dd, J=17.1, 1.9 Hz, 1H), 3.81 (dt, J=13,4, 3.3 Hz, 2H), 3.24-3.08 (m, 2H), 2.23 (d, J=7.6 Hz, 2H), 1.53 (dd, J=10.4, 4.8 Hz, 4H), 1.46 (s, 9H).

### Step 2

A 60% oil dispersion of sodium hydride (2.9 g, 2.0 equiv) was added to a solution of tert-butyl 4-allyl-4-hydroxypiperidine-1-carboxylate (2. 8.8 g, 1 equiv) in anhydrous DMF (100 mL) and the mixture cooled to 0° C. The mixture was warmed to room temperature over 1 hour and methyl 2-(bromomethyl) acrylate (9.8 g, 1.5 equiv) was added dropwise to the solution over 5 minutes. The mixture was aged for 18 hours. A saturated solution of ammonium chloride was added to the reaction mixture and the mixture was diluted with ethyl acetate. The organic phase was separated and washed twice with water then brine, then dried over sodium sulfate, filtered and concentrated in vacuo. The crude mixture was purified using column chromatography on silica gel (0% to 100% ethyl acetate in hexanes). Yield: 4.3 g of tert-butyl 4- {[2-(methoxycarbonyl)prop-2-en-1-yl]oxy}-4-(prop-2-en-1-yl)piperidine-1-carboxylate as a colorless oil (3). LC-MS, [M+Na]⁺: 362.

### Step 3

tert-butyl 4- {[2-(methoxycarbonyl)prop-2-en-1-yl]oxy}-4-(prop-2-en-1-yl)piperidine-1-carboxylate from Step 2 (3, 2.4 g, 1 equiv) in anhydrous 1,2-dichloroethane (75 mL) was combined with benzylidene [1,3-bis(2,4,6-trimethylphenyl)-2-imidazolidinylidene] dichloro-(tricyclohexylphosphine) ruthenium (0.61 g, 10 mol %) and the mixture was heated at 85° C. for overnight. The mixture was cooled to room temperature, then diluted with ethyl acetate and washed with water twice with brine. The separated organic layer was dried over anhydrous sodium sulfate, filtered and evaporated to offer compound 4 as an oil (1.97 g). LC-MS, [M+Na]⁺m/z 334. 1H NMR (400 MHZ. Chloroform-d) 8 6.99 (dt, J=4.3. 2,3 Hz, 1H), 4.30 (m, 2H), 3.76 (m, 2H), 3.74 (s, 3H), 3.15 (m, 2H), 2.14 (dt, J=4.3, 2.9 Hz, 2H), 1.74 (m, 2H), 1.60 (m, 2H), 1.46 (s, 9H),

### Step 4

A MeOH solution of the compound 4 from Step 3 was added palladium on activated carbon catalyst (0.15 eq., 10% purity) under nitrogen. The suspension was degassed under vacuum and purged with hydrogen three times. The mixture was heated at 40° C. for overnight, then filtered and concentrated in vacuo. The crude mixture was purified using column chromatography on silica gel. The product 5 was provided as an oil. [M+Na]⁺m/z 336.

### Step 5

To a stirred solution of compound 5 (5.8 g, 0.0185 mol, 1.0 eq) in dry THF (100 mL) cooled at 0° C. was added LAH (1.05 g, 0.0278 mol, 1.5 eq.) and the mixture was warmed to 0˜25° C. and stirred for 3 h. TLCs were done to detect the process of the reaction. Once the reaction was completed, the mixture was quenched by water (100 mL) and extracted with EtOAc (100 mL*3). The organic phase was separated, dried, concentrated and the residue was purified by silica column chromatography eluting with 50% EtOAc in PE to give compound 6 (3.58 g, 0.015 mol, 68%) as a light yellow oil. ¹H NMR (400 MHZ, Chloroform-d) 8 3.83-3.74 (m, 1H), 3.75-3.58 (m, 2H), 3.55-3.47 (m, 2H), 3.39 (dd, J=11.8, 9.4 Hz, 1H), 3.09 (m, 2H), 2.06 (m, 2H), 1.82-1.52 (m, 5H), 1.43 (s, 9H), 1.33-1.22 (m, 2H),

### Step 6

The compound 6 was further separated by SFC(Instrument: WATERS 150 preparative SFC(SFC-26): Column: ChiralPak AD, 250×30 mm I.D., 10m; Mobile phase: A for CO₂ and B for Methanol; Gradient: B 20%; Flow rate: 150 mL/min; Back pressure: 100 bar) to afford a pure product 6-P1 (2 g) and a pure product 6-P2 (2 g).

### Step 7

To a solution of compound 6-P1 (2 g, 7 mmol, 1 eq.) in dioxane (10 mL) was added 4M HCl-dioxane (10 mL, 42 mmol, 6 eq.) and the mixture was stirred at rt for 2 h. TLC were done to detect the process of the reaction. The solvent was concentrated to give compound 7-P1 as the HCl salt. LC-MS: [M+H]⁺: 186.4.

According to the same procedure, the title compound 7-P2 was provided as HCl salt. LC-MS: [M+H]⁺: 186.4.

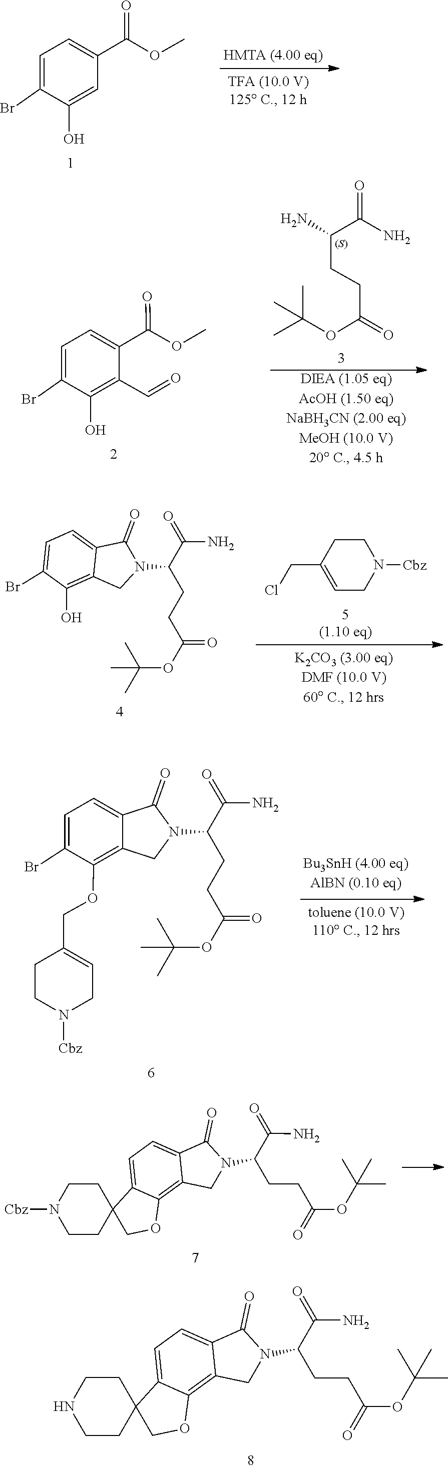

### Step 1

To a solution of compound 1 (20 g, 86.5 mmol, 1 eq) in TFA (200 mL) was added HMTA (48.5 g, 0.346 mol, 4 eq) at 20° C. The mixture was stirred at 125° C. for 12 hrs. TLC (Petroleum ether/Ethyl acetate=5/1) indicated compound 1 was consumed completely and there was desired product. The mixture was quenched with 2N HCl (5 V) and a yellow solid formed. The mixture was stirred for 10 min and then additional water (5 V) was added and stirred for 1 hr. The mixture was filtered. The filter cake was dissolved in DCM and filtered on celite, dried and then remove most of the solvent in vacuo. Compound 2 (14 g, 55 mmol, 61% yield) was obtained as a gray solid, which was indicated by LCMS: [M−H]: 257.6.

### Step 2

Add tert-butyl(S)-4,5-diamino-5-oxopentanoate 3 (17.3 g, 72.4 mmol, 1.05 eq, HCl) in MeOH (300 mL) at 20° C.; Add DIPEA (9.37 g, 72.4 mmol, 12.6 mL, 1.05 eq) to the mixture; The compound 2 (17.8 g, 69.0 mmol, 1 eq) was added into the mixture at the same temperature, followed by adding AcOH (6.22 g, 103 mmol, 5.92 mL, 1.5 eq). After 1.5 hrs, NaBH₃CN (8.67 g, 138 mmol, 2 eq) was added to the mixture in portions and the mixture was stirred at 20° C. for 3 hrs. TLC (Petroleum ether/Ethyl acetate=0/1, R_{f}=0.8) indicated compound 2 was consumed completely and LCMS indicated there was desired product. The reaction mixture was quenched by addition H₂O (200 mL) at 20° C., and then concentrated under reduced pressure to remove MeOH. Then the mixture was extracted with EtOAc (150 mL×3). The combined organic layers were washed with brine (200 mL×2), dried over Na₂SO₄, filtered and concentrated under reduced pressure to give a residue. The residue was purified by column chromatography (Petroleum ether/Ethyl acetate=1/1 to Ethyl acetate). The title compound 4 (20 g, 72% yield) was obtained as a yellow solid, which was detected LCMS: [M+H]⁺: 413.2.

### Step 3

Compound 4 (5 g, 12.1 mmol, 1 eq) was added in DMF (50 mL) at 20° C.; The K₂CO₃ (5.02 g, 36.3 mmol, 3 eq) and compound 5 (2.94 g, 12.7 mmol, 1.05 eq) were added to the mixture and stirred at 60° C. for 12 hrs. TLC (Petroleum ether/Ethyl acetate=0/1) showed that compound 4 was consumed and the desired product was detected by LCMS. The mixture was concentrated under reduced pressure to give a residue, which was added water (100 mL). The product was extracted with DCM (50 mL×3). The organic layers were dried over Na₂SO₄ and concentrated under reduced pressure to give a residue. The residue was purified by column chromatography on silica gel (petroleum ether/EtOAc=1/1 to 0/1). The desired product compound 6 (4.0 g, 6.57 mmol, 54% yield) was obtained as a yellow solid. LC-MS (m/z): [M+H]⁺=642.40.

Step 4

Add compound 6 (660 mg, 1 mmol, 1 eq), Bu₃SnH (1228 mg, 4 mmol) and AIBN (16.4 mg, 0.1 mmol, 0.1 eq) in toluene (5 mL) at 25° C. and then stir the mixture at 110° C. for 12 h. After the reaction completed, the mixture was quenched by addition saturated potassium fluoride solution and stirred for 1 h. The product was extracted with EtOAc. The organic layers were dried over Na₂SO₄ and concentrated under reduced pressure to give a residue. The residue was purified by column chromatography on silica gel to afford the title compound 7 as a yellow solid (400 mg, 69% yield). LC-MS: [M+H]⁺: 564; 1H NMR (400 MHZ, Chloroform-d) 8 7.40 (d. J=7.6 Hz, 1H), 7.39-7.29 (m, 5H), 7.18 (d, J=7.6 Hz, 1H), 6.52 (brs, 1H), 5,67 (brs, 1H), 5.16 (s, 2H), 4.89 (m. 1H), 4.52 (s, 2H), 4.48 (d. J=17.3 Hz, 1H), 4.38 (d. J=17.3 Hz, 1H), 4.20 (m, 2H), 2.95 (m, 2H), 2.40-2.06 (m, 4H), 1.88 (m, 2H), 1.74 (m, 2H), 1.40 (s, 9H).

### Step 5

A 2,2,2-Trifluoroethanol solution of the 7 (500 mg) from Step 4 was added palladium on activated carbon catalyst (0.15 eq., 10% purity) under nitrogen. The suspension was degassed under vacuum and purged with hydrogen three times. The mixture was heated at 25° C. for 3 h, then filtered and concentrated in vacuo. The residue (8) was used directly for next step. LC-MS: [M+H]⁺: 430.

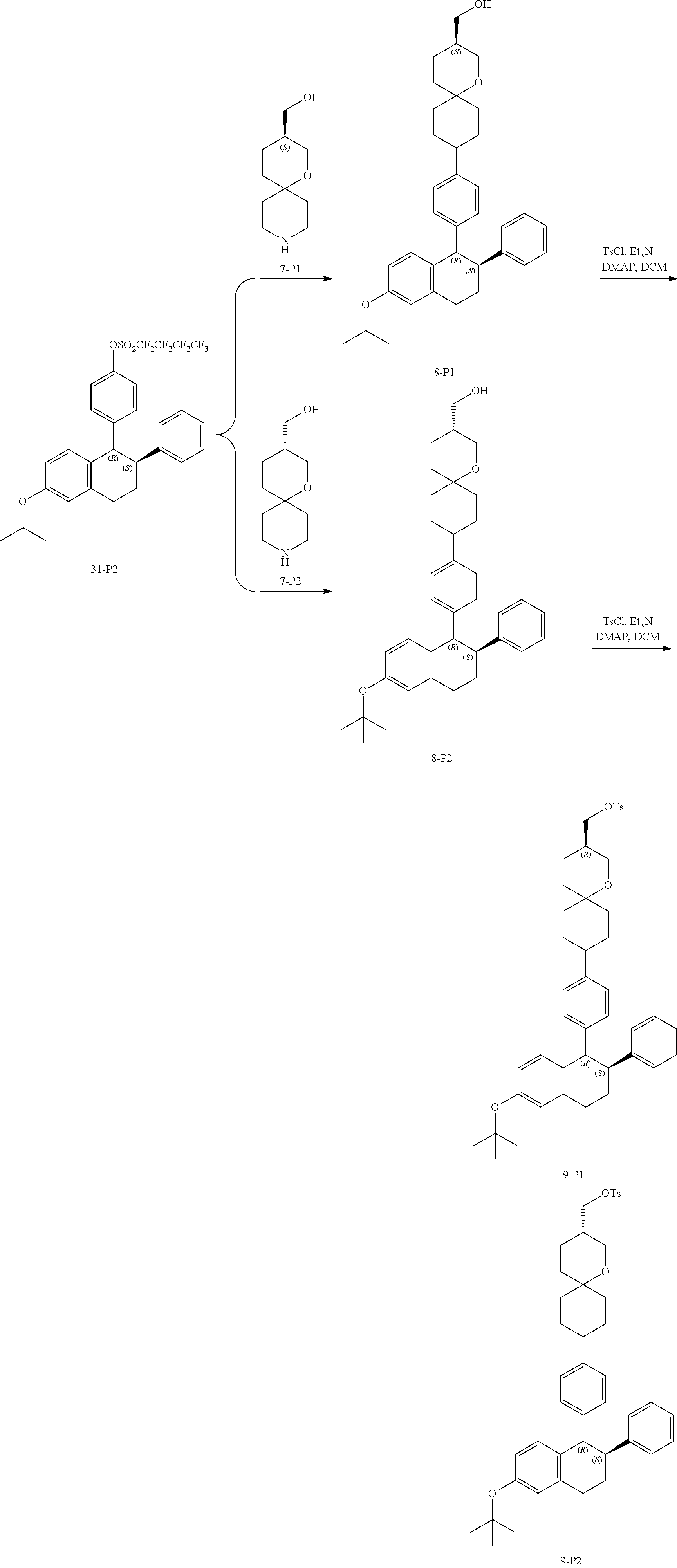

### Step 1

A round bottomed flask equipped with a stirrer bar was charged with a mixture of compound 31-P2 (650 mg, 1 mmol), chiral amine 7-P1 (222 mg, 1,2 mmol), cesium carbonate (650 mg, 2 mmol), and t-BuXphoxPd-G (III) (CAS: 1447963-75-8, 80 mg, 0.1 mmol). The flask was evacuated and back-filled with nitrogen (x 3), t-Amyl alcohol (10 mL) was added and the mixture stirred at 100° C. for 10 hours. The cooled reaction mixture was diluted with EtOAc and filtered through Celite™ to remove insoluble material. The filtrate was washed with water, saturated aqueous sodium chloride and then dried over magnesium sulfate, filtered and the filtrate concentrated. The crude material was purified by flash silica chromatography, elution gradient MeOH in DCM. Pure fractions were combined and concentrated to afford compound 8-P1 (135 mg, 25%). LC-MS: [M+H]⁺: 540.

According to the same procedure, chiral amine 7-P2 was applied to afford compound 8-P2, LC-MS: [M+H]⁺: 540.

### Step 2

To a solution of compound 8-P1 (185 mg, 0.34 mmol, 1 eq.) in DCM (5 mL) was added TEA (0.68 mmol, 2 eq.) and DMAP (0.04 mmol, 0.2 eq.). The flask was evacuated and back-filled with nitrogen. TsCl (0.51 mmol, 1.5 eq.) was added and the mixture stirred at R.T, overnight. TLC indicated the starting material was consumed completely. The reaction mixture was quenched by addition of saturated NH₄Cl solution, and extracted with ethyl acetate. The combined organic layers were washed with brine, dried over sodium sulfate, filtered and concentrated under reduced pressure. The crude material was purified by flash silica chromatography to give compound 9-P1 (141 mg, 60%). LC-MS: [M+H]⁺: 694.

According to the same procedure, the title compound 9-P2 was provided as a white solid. LC-MS: [M+H]⁺: 694.

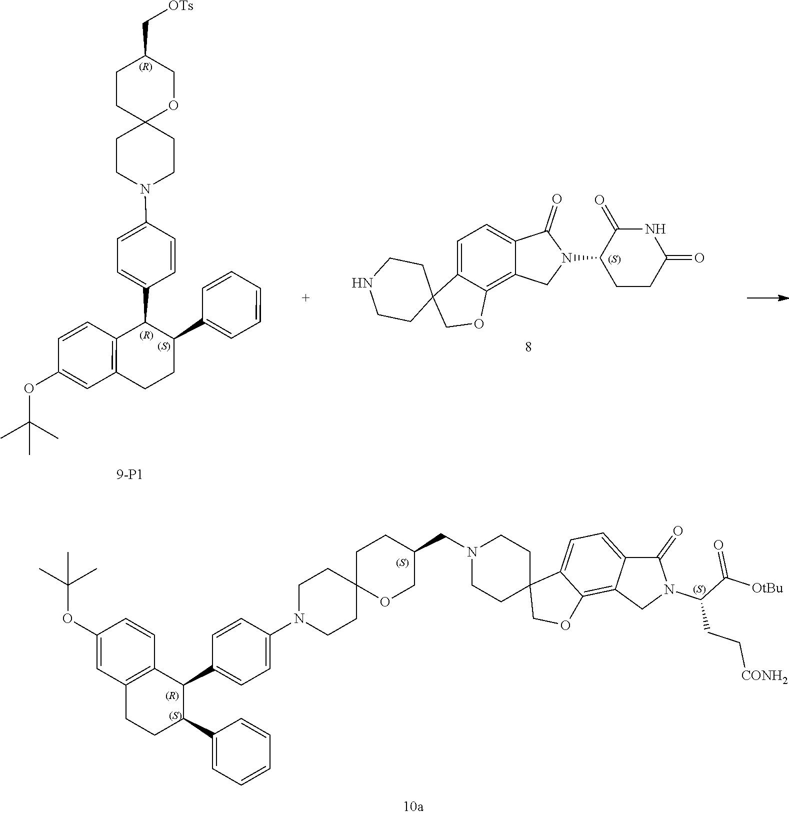

To a solution of compound 9-P1 (160 mg, 0.235 mmol, 1 eq.) in acetonitrile (2 mL) was added 8 (111 mg, 0.258 mmol, 1,1 eq.), KI (40 mg, 0.235 mmol, 1.0 eq.) and diisopropylethylamine (0.12 mL, 3 eq.). The mixture was stirred at 90° C. for 24 hours. LC-MS showed the reaction was completed. The mixture was diluted with water and extracted with ethyl acetate. The combined organic layers was washed wash brine, dried with anhydrous sodium sulfate, filtered and the filtrate was concentrated in vacuum. The residue was purified was purified by flash silica chromatography (DCM:MeOH=10:1) to give the title compound 10a (155 mg, 70%), LC-MS: [M+H]⁺: 951.5.

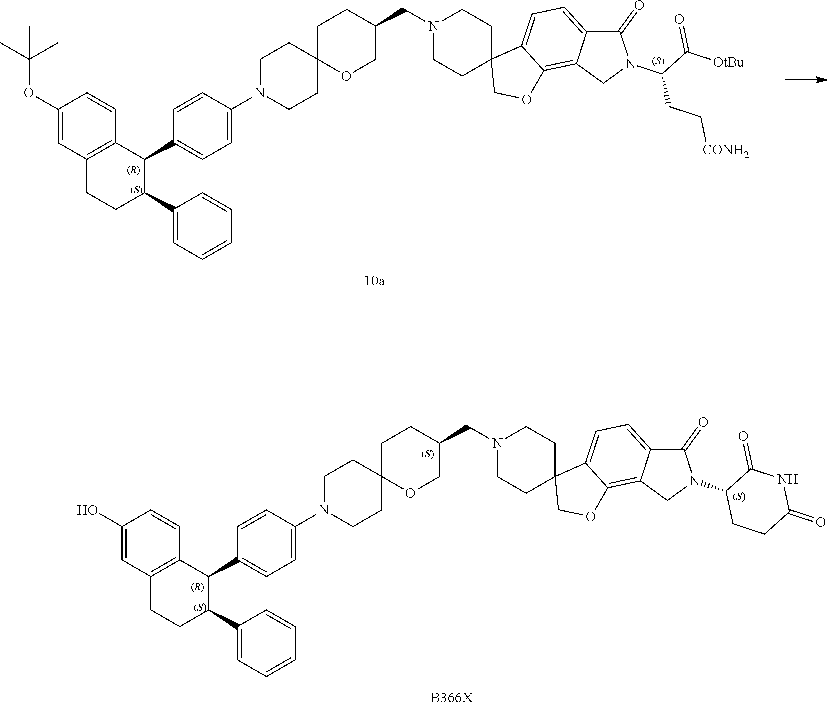

To a solution of compound 10a (100 mg, 0.11 mmol, 1 eq.) in acetonitrile (3 mL) was added benzenesulfonic acid (70 mg, 0.44 mmol, 4 eq.). The mixture was stirred at 80° C. for 3 hours. LC-MS showed the reaction was completed. The mixture was concentrated in vacuum. The residue was purified by preparative reverse phase HPLC(ACN:H₂O=25-55% (1% % TFA) to provide the title compound B366X (53 mg, 60%). LC-MS: [M+H]⁺: 821.4.

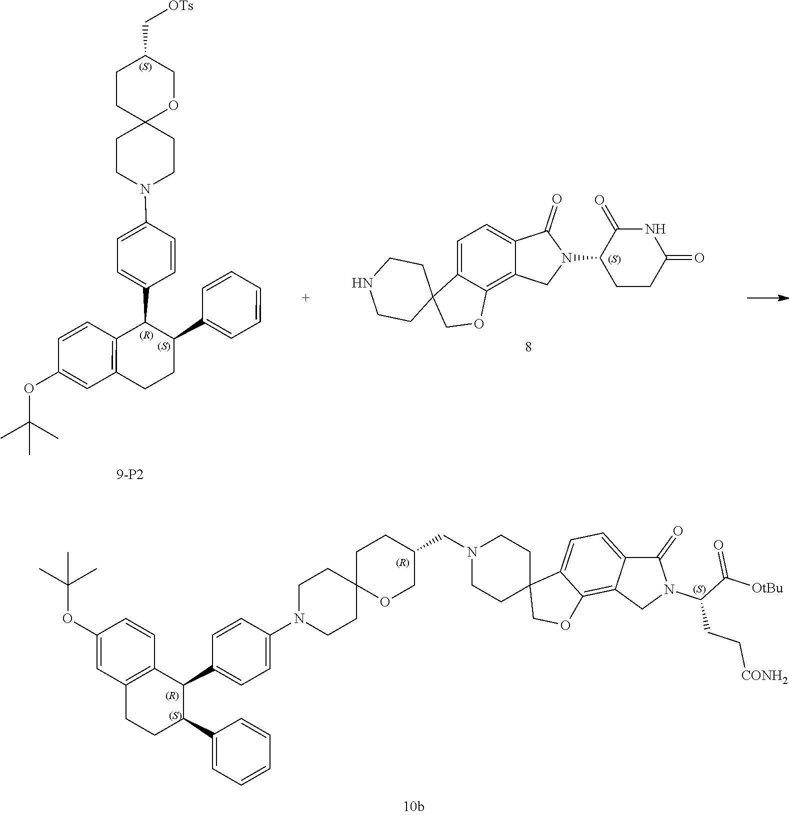

To a solution of compound 9-P2 (160 mg, 0.235 mmol, 1 eq.) in acetonitrile (2 mL) was added 8 (111 mg, 0.258 mmol, 1,1 eq.), KI (40 mg, 0.235 mmol, 1.0 eq.) and diisopropylethylamine (0.12 mL, 3 eq.). The mixture was stirred at 90° C. for 24 hours. LC-MS showed the reaction was completed. The mixture was diluted with water and extracted with ethyl acetate. The combined organic layers was washed wash brine, dried with anhydrous sodium sulfate, filtered and the filtrate was concentrated in vacuum. The residue was purified was purified by flash silica chromatography (DCM:MeOH=10:1) to give the title compound 10b, LC-MS: [M+H]⁺: 951.5.

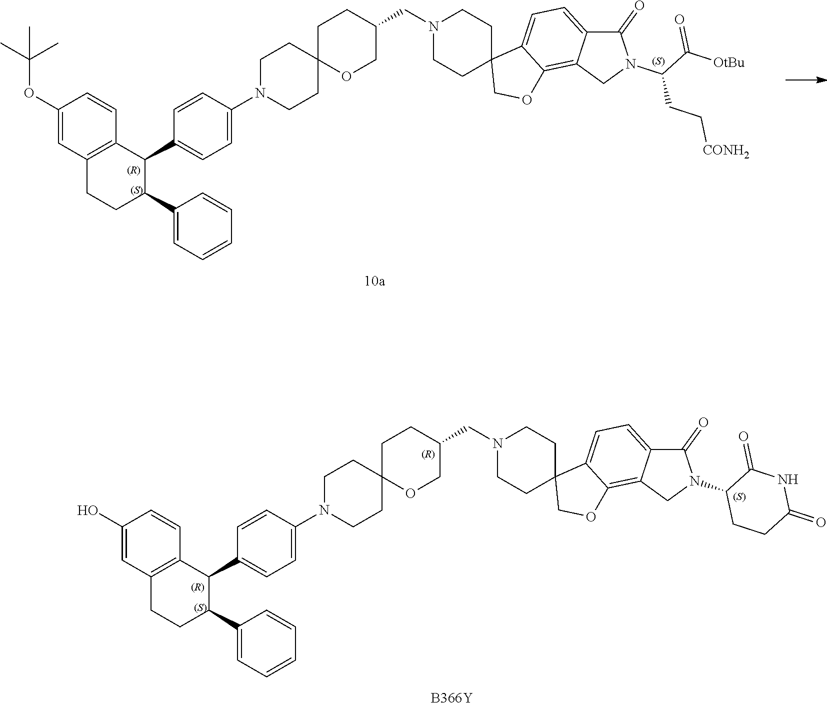

To a solution of compound 10b (100 mg, 0.11 mmol, 1 eq.) in acetonitrile (3 mL) was added benzenesulfonic acid (70 mg, 0.44 mmol, 4 eq.). The mixture was stirred at 80° C. for 3 hours. LC-MS showed the reaction was completed. The mixture was concentrated in vacuum. The residue was purified by preparative reverse phase HPLC(ACN:H₂O=25-55% (1% c TFA) to provide the title compound B366Y (53 mg, 60%). LC-MS: [M+H]⁺: 821.4.

### Compound B383. (S)-3-(5′-fluoro-1-((1-(4-((1R,2S)-6-hydroxy-2-phenyl-1,2,3,4-tetrahydronaphthalen-1-yl)phenyl)piperidin-4-yl)methyl)-6′-oxo-6′,8′-dihydro-3′H,7′H-spiro[piperidine-4,2′-[1,4]dioxino[2,3-f]isoindol]-7′-yl)piperidine-2,6-dione

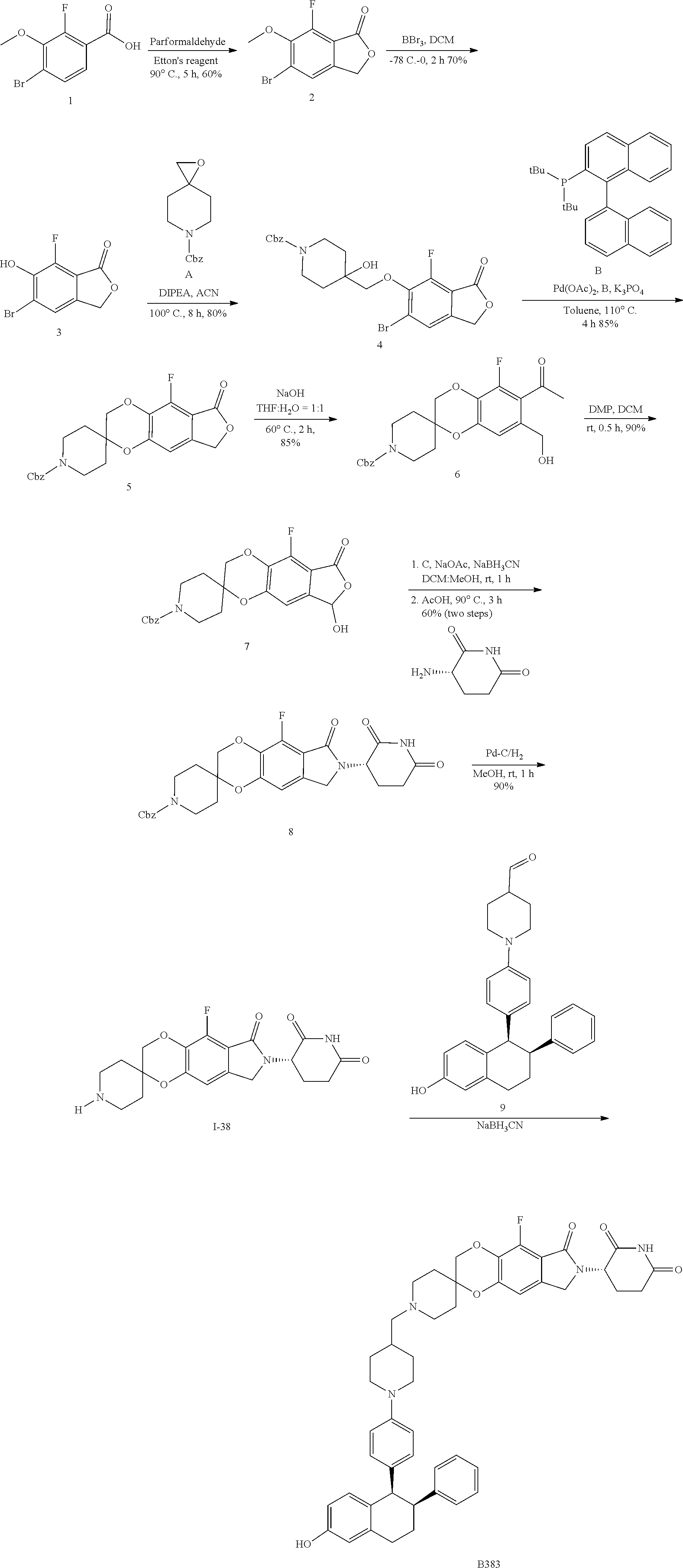

### Step 1:5-bromo-7-fluoro-6-methoxyisobenzofuran-1 (3H)-one

To a 100 mL round-bottom flask, Eaton's reagent (30 mL), compound 4-bromo-3-methoxybenzoic acid (1, 5 μm, 21.83 mmol) and Paraformaldehyde (1.96 g, 65 mmol) were added in an ice bath. The resulting mixture was stirred and heated to 50° C. for overnight. After being cooled to room temperature, the reaction mixture was poured into ice-cold water (100 mL) and extracted with dichloromethane (3×60 mL). The combined organic layer was washed with water, saturated NaHCO₃ and brine, dried over anhydrous Na₂SO₄ and concentrated in vacuo, followed purification by silica gel chromatography to give compound 2 in 70% yield.

### Step 2:5-bromo-7-fluoro-6-hydroxyisobenzofuran-1 (3H)-one

Over a solution of 5-bromo-6-methoxyisobenzofuran-1 (3H)-one (2 g, 8.29 mmol) in dry CH₂Cl₂ (36 mL) under N₂ atmosphere at −20° C. was added boron tribromide (16.6 mL 1M DCM, 16.6 mmol). Then the solution was stirred at rt for overnight. Next, the reaction was quenched adding a saturated solution of NaHCO₃ (15 mL). The aqueous phase was extracted with CH₂Cl₂ (3×30 mL), and the organic phases were combined, dried over sodium sulfate, filtered and the solvent was removed under reduced pressure. The reaction crude was purified by flash chromatography (20% EtOAc/hexane) affording 3 as a white solid (1.32 g, 70% yield). Step 3: benzyl 4-(((6-bromo-4-fluoro-3-oxo-1,3-dihydroisobenzofuran-5-yl)oxy)methyl)-4-hydroxypiperidine-1-carboxylate

To a solution of 3 (1 eq.) in DMF (5 mL/mmol), 10 eq, of DIPEA and 1.5 eq, of epoxide were added into the flask. The reaction was heated to 100° C. The reaction was monitored by UPLC-MS. Concentrated directly and purify by silica gel chromatography to give 4 (96% yield). LC/MS (ESI) m/z: 494.09 (M+H)

### Step 4: benzyl 5′-fluoro-6′-oxo-6′,8′-dihydro-3′H-spiro[piperidine-4,2′-[1,4]dioxino[2,3-flisobenzofuran]-1-carboxylate

To a solution of 4 (1 eq.) in toluene (5 mL/mmol). Pd(OAc)₂ (0.1 eq.), [1,1′-binaphthalen]-2-yldi-tert-butylphosphane (0.1 eq.) and K₃PO₄ (3 eq.) was added into the flask under N₂. The reaction was heated to 140° C. under N₂ for 4h. TLC showed reaction was completed. Quenched with saturated NaHCO₃, the organic phase was separated. Ethyl acetate was added to the mixture, the resulting mixture was washed by brine. The combined organic phase was dried by MgSO₄. Filtered and concentrated in vacuum. The residue was purified by silica gel chromatography to give 5 in (70% yield). LC/MS (ESI) m/z: 414.12 (M+H)

### Step 5: 1′-((benzyloxy) carbonyl)-5-fluoro-7-(hydroxymethyl)-3H-spiro[benzo[b][1,4]dioxine-2,4′-piperidine]-6-carboxylic acid

To a solution of(S)-2-((benzyloxy)methyl)-2,3-dihydro-[1,4]dioxino[2,3-f]isobenzofuran-6 (8H)-one (5, eq.) in tetrahydrofuran and water (1:1) was added sodium hydroxide (5 eq.). The mixture was stirred at 20° C. for 16 h. TLC (ethyl acetate: hexane=1:1) showed reaction was complete. The mixture was adjusted to pH=5 with aq, hydrochloric acid (1 M) and extracted with ethyl acetate (10 mL×3). The organic layer was washed with brine (10)×2 mL) and dried over sodium sulfate. The crude material (6) was not further purified and used as crude for the next steps. LC/MS (ESI) m/z: 432.12 (M+H)

### Step 6: benzyl 5′-fluoro-8′-hydroxy-6′-oxo-6′,8′-dihydro-3′H-spiro[piperidine-4,2′-[1,4]dioxino[2,3-f]isobenzofuran]-1-carboxylate

To a solution of 6 (1.0 eq) in DCM (10 mL) was added DMP (1,2 eq.) at 0° C. and stirred it for 30 mins. TLC (ethyl acetate: hexane=1:1) showed reaction was complete. The reaction mixture was then diluted with DCM, washed with brine, dried over and concentrated under reduced pressure. The residue was purified by silica gel chromatography to give 7 in (70% yield). LC/MS (ESI) m/z: 430.12 (M+H),

### Step 7&8: benzyl(S)-7′-(2,6-dioxopiperidin-3-yl)-5′-fluoro-6′-oxo-7′,8′-dihydro-3′H,6′H-spiro[piperidine-4,2′-[1,4]dioxino[2,3-f]isoindole]-1-carboxylate

A mixture of 7 (1.0 eq, (S)-3-aminopiperidine-2,6-dione (1.5 eq) and NaOAc (3 eq) was dissolved in methanol: DCM (1:1), and kept stirring at rt for 20 min. Then NaBH₃CN (2.0 eq) was added, 2 h Later, UPLC-MS showed the starting material 7 was completely conversion and a new main peak 8 with desired MS formed. LC/MS (ESI) m/z: 542.16 (M+H). The crude compound 8 (1.0 eq., 440 mg) was dissolved in CH₃CN (4 mL) and was treated with HOAc (15 eq.). The rection was heated at 60° C. and stirred for 2h. Next, the reaction mixture was quenched with water and concentrated under reduced pressure to give a residue which was purified by pre-HPLC to give 8 in (70% yield), ¹H NMR (400 MHZ, Chloroform-d) δ 8.02 (s, 1H), 7.47-7.32 (m, 5H), 6.81 (s, 1H), 5.18 (s, 3H), 4.48-4,20 (m, 2H), 4.02 (s, 2H), 3.33 (t, J=12.5 Hz, 2H), 3.05-2,75 (m, 4H), 2.45-2.15 (m, 2H), 1.85 (s, 2H), 1.67 (s, 2H), 13C NMR (101 MHZ, CDCl₃) δ 171,47, 169.73, 166.88, 155.37, 149.27, 147.61, 146.68, 136.42, 134.67, 131.63, 131.50, 128.56 (two peaks), 128.19, 127.99 (two peaks), 112.12, 112.02, 107.52, 72.74, 70.78, 67.52, 51.79, 46.71, 39.06. 31,44, 23,24. LC/MS (ESI) m/z: 523.15 (M+H),

### Step 9: (S)-3-(5′-fluoro-6′-oxo-6′,8′-dihydro-3′H,7′H-spiro[piperidine-4,2′-[1,4]dioxino[2,3-flisoindol]-7′-yl)piperidine-2,6-dione

To a stirred solution of compound 8 in methanol at room temperature Pd-C was added and the reaction was stirred for another 1 h. UPLC mass chromatography showed the completion of the reaction. Catalyst was filtered out and washed with MeOH. Organic solvent was evaporated, and the product (I-38) was used for the next step. LC/MS (ESI) m/z: 390.12 (M+H)

### Step 10: (S)-3-(5′-fluoro-1-((1-(4-((1R,2S)-6-hydroxy-2-phenyl-1,2,3,4-tetrahydronaphthalen-1-yl)phenyl)piperidin-4-yl)methyl)-6′-oxo-6′,8′-dihydro-3′H,7′H-spiro[piperidine-4,2′-[1,4]dioxino[2,3-f]isoindol]-7′-yl)piperidine-2,6-dione

To a solution of I-38 (1,2 equiv) in Methanol:DCM (4:1) was added NaOAc (2 equiv) and the mixture was stirred at room temperature for 5 mins. The aldehyde 9 (1 equiv) in MeOH:DCM (4:1) was then added to the reaction mixture and stirred for another 10 mins. Sodiumcyanoborohydride (2 equiv) and Acetic acis was then added to the reaction sequentially and stirred for another 15 minutes. UPLC chromatography showed the completion of the reaction, and the product was purified using preparative HPLC. LC/MS (ESI) m/z: 785.41 (M+H)

### Compound B394. (S)-3-(1-((7-(4-((1R,2S)-6-hydroxy-2-phenyl-1,2,3,4-tetrahydronaphthalen-1-yl)phenyl)-7-azaspiro[3.5]nonan-2-yl)methyl)-6′-oxo-6′,8′-dihydro-3′H,7′H-spiro[azetidine-3,2′-[1,4]dioxino[2,3-f]isoindol]-7′-yl)piperidine-2,6-dione

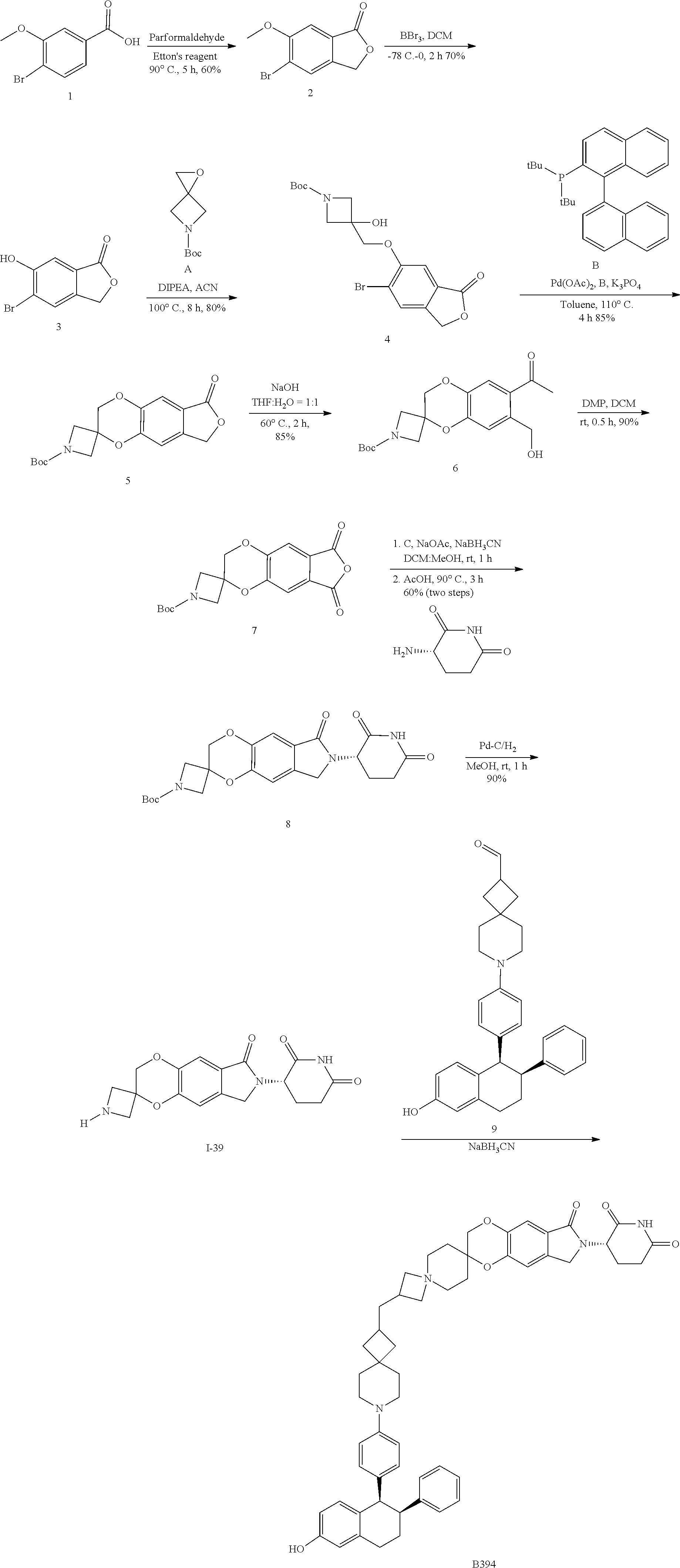

### Step 1:5-bromo-6-methoxyisobenzofuran-1 (3H)-one

To a 100 mL round-bottom flask, Eaton's reagent (30 mL), compound 4-bromo-3-methoxybenzoic acid (1, 5 μm, 21.83 mmol) and Paraformaldehyde (1.96 g, 65 mmol) were added in an ice bath. The resulting mixture was stirred and heated to 50° C. for overnight. After being cooled to room temperature, the reaction mixture was poured into ice-cold water (100 mL) and extracted with dichloromethane (3×60 mL). The combined organic layer was washed with water, saturated NaHCO₃ and brine, dried over anhydrous Na₂SO₄ and concentrated in vacuo, followed purification by silica gel chromatography to give compound 2 in 70% yield.

### Step 2:5-bromo-6-hydroxyisobenzofuran-1 (3H)-one

Over a solution of 5-bromo-6-methoxyisobenzofuran-1 (3H)-one (2 g, 8.29 mmol) in dry CH₂Cl₂ (36 mL) under N₂ atmosphere at −20° C. was added boron tribromide (16.6 mL 1M DCM, 16.6 mmol). Then the solution was stirred at rt for overnight. Next, the reaction was quenched adding a saturated solution of NaHCO₃ (15 mL). The aqueous phase was extracted with CH₂Cl₂ (3×30 mL), and the organic phases were combined, dried over sodium sulfate, filtered and the solvent was removed under reduced pressure. The reaction crude was purified by flash chromatography (20% EtOAc/hexane) affording 3 as a white solid (1.32 g, 70% yield).

### Step 3: tert-butyl 3-(((6-bromo-3-oxo-1,3-dihydroisobenzofuran-5-yl)oxy)methyl)-3-hydroxyazetidine-1-carboxylate

To a solution of 3 (1 eq.) in DMF (5 mL/mmol), 10 eq, of DIPEA and 1.5 eq, of epoxide were added into the flask. The reaction was heated to 100° C. The reaction was monitored by UPLC-MS. Concentrated directly and purify by silica gel chromatography to give 4 (96% yield). LC/MS (ESI) m/z: 414.12 (M+H)

### Step 4: tert-butyl 6′-oxo-6′,8′-dihydro-3′H-spiro[azetidine-3,2′-[1,4]dioxino[2,3-flisobenzofuran]-1-carboxylate

To a solution of 4 (1 eq.) in toluene (5 mL/mmol). Pd(OAc)₂ (0.1 eq.), [1,1′-binaphthalen]-2-yldi-tert-butylphosphane (0.1 eq.) and K₃PO₄ (3 eq.) was added into the flask under N₂. The reaction was heated to 140° C. under N₂ for 4h. TLC showed reaction was completed. Quenched with saturated NaHCO₃, the organic phase was separated. Ethyl acetate was added to the mixture, the resulting mixture was washed by brine. The combined organic phase was dried by MgSO₄. Filtered and concentrated in vacuum. The residue was purified by silica gel chromatography to give 5 in (70% yield). LC/MS (ESI) m/z: 334.15 (M+H)

### Step 5:1-(tert-butoxycarbonyl)-7′-(hydroxymethyl)-3′H-spiro[azetidine-3,2′-benzo[b][1,4]dioxine]-6′-carboxylic acid

To a solution of(S)-2-((benzyloxy)methyl)-2,3-dihydro-[1,4]dioxino[2,3-f]isobenzofuran-6 (8H)-one (5, 1 eq.) in tetrahydrofuran and water (1:1) was added sodium hydroxide (5 eq.). The mixture was stirred at 20° C. for 16 h. TLC (ethyl acetate: hexane=1:1) showed reaction was complete. The mixture was adjusted to pH=5 with aq, hydrochloric acid (1 M) and extracted with ethyl acetate (10 mL×3). The organic layer was washed with brine (10×2 mL) and dried over sodium sulfate. The crude material (6) was not further purified and used as crude for the next steps.

LC/MS (ESI) m/z: 352.12 (M+H)

### Step 6: tert-butyl 8′-hydroxy-6′-oxo-6′,8′-dihydro-3′H-spiro[azetidine-3,2′-[1,4]dioxino[2,3-flisobenzofuran]-1-carboxylate

To a solution of 6 (1.0 eq) in DCM (10 mL) was added DMP (1,2 eq.) at 0° C. and stirred it for 30 mins. TLC (ethyl acetate: hexane=1:1) showed reaction was complete. The reaction mixture was then diluted with DCM, washed with brine, dried over and concentrated under reduced pressure. The residue was purified by silica gel chromatography to give 7 in (70% yield). LC/MS (ESI) m/z: 350.15 (M+H),

### Step 7&8: tert-butyl(S)-7′-(2,6-dioxopiperidin-3-yl)-6′-oxo-7′,8′-dihydro-3′H,6′H-spiro[azetidine-3,2′-[1,4]dioxino[2,3-f]isoindole]-1-carboxylate

A mixture of 7 (1.0 eq), (S)-3-aminopiperidine-2,6-dione (1.5 e eq) and NaOAc (3.0 eq) was dissolved in methanol: DCM (1:1), and kept stirring at rt for 20 min. Then NaBH₃CN (2.0 eq.) was added, 2 h Later, UPLC-MS showed the starting material 7 was completely conversion and a new main peak 8 with desired MS formed. LC/MS (ESI) m/z: 462.16 (M+H). The crude compound 8 (1.0 eq) was dissolved in CH₃CN (4 mL) and was treated with HOAc (15 eq.). The rection was heated at 60° C. and stirred for 2h. Next, the reaction mixture was quenched with water and concentrated under reduced pressure to give a residue which was purified by pre-HPLC to give I-39 in (70% yield), ¹H NMR (400 MHZ, Chloroform-d) 8 7.92 (s, 1H), 7.43 (s, 1H), 7.01 (d. J=0.8 Hz, 1H), 5.20 (dd, J=13.2, 5.2 Hz, 1H), 4.45-4,21 (m, 4H), 4.14-3.94 (m, 5H), 3.07-2.71 (m, 2H), 2.52-2.16 (m, 2H), 1.57 (s, 9H).

LC/MS (ESI) m/z: 444.15 (M+H),

### Step 9: (S)-3-(6′-oxo-6′,8′-dihydro-3′H,7′H-spiro[azetidine-3,2′-[1,4]dioxino[2,3-f]isoindol]-7′-yl)piperidine-2,6-dione

To a stirred solution of compound 8 in methanol at room temperature Pd-C was added and the reaction was stirred for another 1 h. UPLC mass chromatography showed the completion of the reaction. Catalyst was filtered out and washed with MeOH. Organic solvent was evaporated, and the product (I-39) was used for the next step. LC/MS (ESI) m/z: 344.12 (M+H)

### Step 10: (S)-3-(1-((7-(4-((1R,2S)-6-hydroxy-2-phenyl-1,2,3,4-tetrahydronaphthalen-1-yl)phenyl)-7-azaspiro[3.5]nonan-2-yl)methyl)-6′-oxo-6′,8′-dihydro-3′H,7′H-spiro[azetidine-3,2′-[1,4]dioxino[2,3-f]isoindol]-7′-yl)piperidine-2,6-dione

To a solution of I-39 (1,2 equiv) in Methanol:DCM (4:1) was added NaOAc (2 equiv) and the mixture was stirred at room temperature for 5 mins. The aldehyde 9 (1 equiv) in MeOH:DCM (4:1) was then added to the reaction mixture and stirred for another 10 mins. Sodiumcyanoborohydride (2 equiv) and Acetic acis was then added to the reaction sequentially and stirred for another 15 minutes. UPLC chromatography showed the completion of the reaction, and the product B394 was purified using preparative HPLC. LC/MS (ESI) m/z: 779.42 (M+H)

TABLE 7
Characterization Data for “B” Compounds

| Compound No. | (M + H)⁺ |
|---|---|
| B1 | 681.34 |
| B2 | 695.32 |
| B3 | 764.41 |
| B4 | 736.23 |
| B5 | 764.34 |
| B6 | 810.36 |
| B7 | 792.37 |
| B8 | 806.32 |
| B9 | 764.34 |
| B10 | 778.35 |
| B11 | 781.92 |
| B12 | 792.43 |
| B13 | 739.31 |
| B14 | 779.34 |
| B15 | 807.37 |
| B16 | 843.33 |
| B17 | 764.04 |
| B18 | 793.49 |
| B19 | 782.51 |
| B20 | 778.35 |
| B21 | 778.35 |
| B22 | 778.35 |
| B23 | 792.37 |
| B24 | 792.37 |
| B25 | 792.37 |
| B26 | 810.36 |
| B27 | 810.36 |
| B28 | 764.12 |
| B29 | 764.34 |
| B30 | 792.37 |
| B31 | 796.34 |
| B32 | 796.34 |
| B33 | 796.34 |
| B34 | 814.34 |
| B35 | 778.35 |
| B36 | 757.30 |
| B37 | 750.32 |
| B38 | 757.30 |
| B39 | 820.40 |
| B40 | 820.40 |
| B41 | 792.37 |
| B42 | 792.37 |
| B43 | 782.33 |
| B44 | 782.33 |
| B45 | 825.38 |
| B46 | 828.35 |
| B47 | 798.42 |
| B48 | 834.40 |
| B49 | 770.38 |
| B50 | 820.23 |
| B51 | 820.05 |
| B52 | 837.39 |
| B53 | 810.36 |
| B54 | 811.35 |
| B55 | 796.34 |
| B56 | 806.38 |
| B57 | 806.38 |
| B58 | 789.33 |
| B59 | 761.31 |
| B60 | 739.31 |
| B61 | 750.28 |
| B62 | 764.16 |
| B63 | 782.13 |
| B64 | 778.28 |
| B65 | 750.30 |
| B66 | 796.17 |
| B67 | 782.21 |
| B68 | 782.22 |
| B69 | 768.19 |
| B70 | 782.16 |
| B71 | 754.09 |
| B72 | 754.32 |
| B73 | 768.18 |
| B74 | 782.20 |
| B75 | 806.20 |
| B76 | 778.16 |
| B77 | 792.14 |
| B78 | 737.00 |
| B79 | 778.20 |
| B80 | 764.26 |
| B81 | 793.14 |
| B82 | 794.30 |
| B83 | 778.20 |
| B84 | 778.12 |
| B85 | 794.24 |
| B86 | 793.04 |
| B87 | 794.08 |
| B88 | 792.40 |
| B89 | 761.15 |
| B90 | 760.15 |
| B91 | 760.15 |
| B92 | 761.04 |
| B93 | 738.03 |
| B94 | 752.20 |
| B95 | 778.28 |
| B96 | 778.25 |
| B97 | 778.25 |
| B98 | 778.07 |
| B99 | 764.03 |
| B100 | 764.53 |
| B101 | 764.25 |
| B102 | 750.24 |
| B103 | 750.23 |
| B104 | 760.91 |
| B105 | 778.24 |
| B106 | 765.12 |
| B107 | 765.20 |
| B108 | 764.22 |
| B109 | 820.24 |
| B110 | 820.27 |
| B111 | 792.19 |
| B112 | 792.17 |
| B113 | 791.99 |
| B114 | 806.18 |
| B115 | 778.30 |
| B116 | 778.22 |
| B117 | 759.13 |
| B118 | 764.23 |
| B119 | 764.20 |
| B120 | 822.11 |
| B121 | 794.15 |
| B122 | 779.10 |
| B123 | 779.10 |
| B124 | 778.14 |
| B125 | 778.12 |
| B126 | 779.11 |
| B127 | 779.13 |
| B128 | 778.19 |
| B129 | 778.19 |
| B130 | 792.23 |
| B131 | 792.23 |
| B132 | 806.21 |
| B133 | 792.22 |
| B134 | 765.07 |
| B135 | 751.95 |
| B136 | 822.14 |
| B137 | 765.87 |
| B138 | 816.40 |
| B139 | 816.37 |
| B140 | 849.47 |
| B141 | 863.48 |
| B142 | 821.36 |
| B143 | 821.51 |
| B144 | 781.47 |
| B145 | 781.27 |
| B146 | 849.46 |
| B147 | 849.45 |
| B148 | 850.42 |
| B149 | 851.38 |
| B150 | 768.25 |
| B151 | 768.24 |
| B152 | 807.35 |
| B153 | 807.38 |
| B154 | 807.40 |
| B155 | 807.34 |
| B156 | 807.36 |
| B157 | 807.34 |
| B158 | 850.36 |
| B159 | 851.51 |
| B160 | 821.35 |
| B161 | 821.37 |
| B162 | 834.41 |
| B163 | 750.33 |
| B164 | 807.34 |
| B165 | 765.31 |
| B166 | 778.28 |
| B167 | 793.36 |
| B168 | 806.20 |
| B169 | 806.22 |
| B170 | 806.34 |
| B171 | 820.18 |
| B172 | 808.15 |
| B173 | 808.14 |
| B174 | 821.17 |
| B175 | 807.22 |
| B176 | 808.10 |
| B177 | 808.10 |
| B178 | 807.15 |
| B179 | 764.34 |
| B180 | 792.37 |
| B181 | 764.53 |
| B182 | 764.37 |
| B183 | 750.36 |
| B184 | 736.34 |
| B185 | 778.35 |
| B186 | 764.34 |
| B187 | 750.32 |
| B188 | 754.3 |
| B189 | 749.36 |
| B190 | 806.38 |
| B191 | 806.38 |
| B192 | 778.35 |
| B193 | 796.34 |
| B194 | 824.37 |
| B195 | 808.4 |
| B196 | 750.36 |
| B197 | 750.36 |
| B198 | 778.39 |
| B199 | 792.4 |
| B200 | 820.4 |
| B201 | 806.42 |
| B202 | 792.4 |
| B203 | 808.37 |
| B204 | 767.3 |
| B205 | 793.32 |
| B206 | 807.38 |
| B207 | 767.31 |
| B208 | 793.41 |
| B209 | 807.4 |
| B210 | 808.32 |
| B211 | 792.4 |
| B212 | 806.4 |
| B213 | 820.49 |
| B214 | 820.33 |
| B215 | 738.36 |
| B216 | 738.34 |
| B217 | 707.38 |
| B218 | 821.39 |
| B219 | 821.39 |
| B220 | 767.35 |
| B221 | 821.39 |
| B222 | 793.36 |
| B223 | 823.41 |
| B224 | 741.37 |
| B225 | 781.40 |
| B226 | 708.40 |
| B227 | 708.40 |
| B228 | 791.41 |
| B229 | 807.40 |
| B230 | 780.37 |
| B231 | 780.42 |
| B232 | 778.39 |
| B233 | 778.37 |
| B234 | 821.43 |
| B235 | 751.36 |
| B236 | 797.39 |
| B237 | 811.41 |
| B238 | 810.38 |
| B239 | 810.36 |
| B240 | 824.39 |
| B242 | 824.42 |
| B243 | 752.3 |
| B244 | 781.3 |
| B245 | 767.9 |
| B246 | 768.3 |
| B247 | 780.8 |
| B248 | 768.3 |
| B249 | 781.3 |
| B250 | 767.30 |
| B251 | 793.20 |
| B252 | 821.20 |
| B253 | 820.30 |
| B254 | 821.30 |
| B255 | 754.20 |
| B256 | 740.30 |
| B257 | 740.40 |
| B258 | 751.40 |
| B259 | 816.30 |
| B260 | 792.40 |
| B261 | 792.30 |
| B262 | 796.30 |
| B263 | 750.20 |
| B264 | 778.30 |
| B265 | 814.30 |
| B266 | 796.30 |
| B267 | 824.40 |
| B268 | 796.30 |
| B269 | 824.30 |
| B270 | 810.40 |
| B271 | 782.30 |
| B272 | 810.30 |
| B273 | 792.40 |
| B274 | 764.30 |
| B275 | 779.30 |
| B276 | 751.30 |
| B277 | 779.30 |
| B278 | 779.30 |
| B279 | 807.30 |
| B280 | 810.30 |
| B281 | 793.30 |
| B282 | 773.30 |
| B283 | 806.20 |
| B284 | 739.30 |
| B285 | 779.10 |
| B286 | 779.20 |
| B287 | 791.30 |
| B288 | 763.20 |
| B289 | 763.30 |
| B290 | 820.30 |
| B291 | 792.90 |
| B292 | 804.40 |
| B293 | 793.30 |
| B294 | 779.30 |
| B295 | 766.20 |
| B296 | 735.30 |
| B297 | 749.30 |
| B298 | 752.30 |
| B299 | 751.30 |
| B300 | 751.30 |
| B301 | 764.30 |
| B302 | 750.30 |
| B303 | 778.90 |
| B304 | 777.40 |
| B305 | 764.30 |
| B306 | 752.20 |
| B307 | 752.10 |
| B308 | 792.40 |
| B309 | 792.40 |
| B310 | 766.40 |
| B311 | 780.20 |
| B312 | 794.40 |
| B313 | 791.30 |
| B314 | 740.20 |
| B315 | 712.30 |
| B316 | 713.30 |
| B317 | 730.40 |
| B318 | 805.40 |
| B319 | 805.30 |
| B320 | 778.30 |
| B321 | 778.30 |
| B322 | 766.40 |
| B323 | 856.40 |
| B324 | 842.40 |
| B325 | 794.30 |
| B326 | 794.40 |
| B327 | 796.36 |
| B328 | 796.32 |
| B329 | 806.40 |
| B330 | 822.40 |
| B331 | 820.40 |
| B332 | 822.40 |
| B333 | 794.40 |
| B334 | 794.40 |
| B335 | 793.30 |
| B336 | 813.35 |
| B337 | 823.40 |
| B338 | 767.34 |
| B339 | 727.36 |
| B340 | 858.36 |
| B341 | 796.13 |
| B342 | 796.11 |
| B343 | 795.11 |
| B344 | 739.31 |
| B345 | 755.41 |
| B346 | 795.36 |
| B347 | 785.39 |
| B348 | 794.40 |
| B349 | 794.40 |
| B350 | 809.36 |
| B351 | 764.36 |
| B352 | 737.39 |
| B353 | 766.40 |
| B354 | 766.30 |
| B355 | 753.38 |
| B356 | 796.42 |
| B358 | 778.42 |
| B359 | 777.42 |
| B360 | 767.36 |
| B362 | 806.42 |
| B363 | 806.36 |
| B364 | 805.43 |
| B365 | 795.43 |
| B366 | 821.40 |
| B367 | 739.25 |
| B368 | 779.3 |
| B369 | 766.99 |
| B370 | 767.24 |
| B371 | 807.30 |
| B372 | 823.40 |
| B373 | 823.31 |
| B374 | 839.32 |
| B375 | 739.20 |
| B376 | 779.35 |
| B377 | 811.28 |
| B378 | 701.30 |
| B379 | 741.30 |
| B380 | 702.40 |
| B381 | 742.40 |
| B382 | 772.40 |
| B383 | 785.39 |
| B384 | 825.39 |
| B385 | 793.35 |
| B386 | 809.29 |
| B387 | 767.19 |
| B388 | 739.19 |
| B389 | 873.25 |
| B390 | 873.44 |
| B391 | 767.28 |
| B392 | 767.19 |
| B393 | 739.19 |
| B394 | 779.42 |
| B395 | 767.37 |
| B396 | 808.41 |
| B397 | 808.41 |
| B398 | 808.41 |
| B399 | 808.41 |
| B400 | 821.25 |
| B401 | 821.25 |

### II. Biological Assays

### For “A” Compounds

In vitro Assay: IC₅₀ Measurements for binding to CRBN/DDB1

The binding potency was determined using HTRF assay technology (Perkin Elmer). Compounds were serially diluted in DMSO and 0.2 L volume was transferred to white 384-well plate. The reaction was conducted in total volume of 20 μL with addition of 2 nM His tagged CRBN+DDB-DLS7+CXU4 (Wuxi, catalogue #RP210521GA) to compounds followed by addition of 60 nM Fluorescent probe Cy5-labeled Thalidomide (Tenova Pharma, catalogue #T52461), and 0.4 nM of MAb Anti-6HIS Tb cryptate Gold (Cisbio, catalogue #61HI2TLA in the assay buffer (50 mM HEPES pH 7.5, 1 mM TCEP, 0.01% Brij-35, 50 mM NaCl, and 0.1% BSA). After one hour incubation at room temperature, the HTRF signals were read on Envision reader (Perkin Elemer). Data was analyzed using XL fit using four parameters dose response curve to determine IC₅₀s.

TABLE 8
Binding IC₅₀ to CRBN/DDB1

| Compound No. | CRBN HTRF IC₅₀ (nM) |
|---|---|
| A1 | C |
| A2 | C |
| A3 | D |
| A4 | C |
| A5 | D |
| A6 | C |
| A7 | C |
| A8 | B |
| A9 | A |
| A10 | A |
| A11 | B |
| A12 | B |
| A13 | C |
| A14 | C |
| A15 | C |
| A16 | C |
| A17 | C |
| A18 | C |
| A19 | B |
| A20 | B |
| A21 | B |
| A22 | B |
| A23 | A |
| A24 | B |
| A25 | B |
| A26 | B |
| A27 | B |
| A28 | B |
| A29 | B |
| A30 | B |
| A31 | B |
| A32 | B |
| A33 | B |
| A34 | B |
| A35 | B |
| A36 | A |
| A37 | A |
| A38 | B |
| A39 | B |
| A40 | A |
| A41 | A |
| A42 | B |
| A43 | B |
| A44 | D |
| A45 | D |
| A46 | B |
| A47 | B |
| A48 | B |
| A49 | B |
| A50 | B |
| A51 | B |
| A52 | A |
| A53 | A |
| A54 | A |
| A55 | A |
| A56 | B |
| A57 | B |
| A58 | D |
| A59 | B |
| A60 | B |
| A61 | B |
| A62 | B |
| A63 | C |
| A64 | D |
| A65 | B |
| A66 | B |
| A67 | D |
| A68 | A |
| A69 | B |
| A70 | A |
| A71 | B |
| A72 | B |
| A73 | C |
| A74 | A |
| A75 | A |
| A76 | B |
| A77 | B |
| A78 | A |
| A79 | A |
| A80 | A |
| A81 | A |
| A82 | B |
| A83 | B |
| A84 | B |
| A85 | B |
| A86 | B |
| A87 | B |
| A88 | D |
| A89 | D |
| A90 | D |
| A91 | C |
| A92 | D |
| A93 | A |
| A94 | D |
| A95 | A |
| A96 | B |
| A97 | B |
| A98 | B |
| A99 | B |
| A100 | A |
| A101 | D |
| A102 | A |
| A103 | B |
| A104 | D |
| A105 | B |
| A106 | B |
| A107 | D |
| A108 | C |
| A109 | C |
| A110 | B |
| A111 | D |
| A112 | B |
| A113 | B |
| A114 | ND |
| A115 | ND |
| A116 | A |
| A117 | A |
| A118 | C |
| A119 | B |
| A120 | C |
| A121 | C |
| A122 | B |
| A123 | B |
| A124 | C |
| A125 | B |
| A126 | B |
| A127 | ND |
| A128 | B |
| A129 | B |
| A130 | C |
| A131 | A |
| A132 | C |
| A133 | ND |
| A134 | ND |
| A135 | C |
| A136 | C |
| A137 | B |
| A138 | B |
| A139 | B |
| A140 | A |
| A141 | A |
| A142 | A |
| A143 | C |
| A144 | C |
| A145 | B |
| A146 | B |
| A147 | B |
| A148 | A |
| A149 | C |
| A150 | C |
| A151 | C |
| A152 | C |
| A153 | A |
| A154 | A |
| A155 | B |
| A156 | B |
| A157 | C |
| A158 | C |
| A159 | B |
| A160 | B |
| A161 | A |
| A162 | B |
| A163 | B |
| A164 | C |
| A165 | B |
| A166 | ND |
| A167 | ND |
| A168 | ND |
| A169 | ND |
| A170 | ND |
| A171 | ND |
| A172 | ND |

Note:
IC₅₀: “A”: <50 nM; “B”: 50-500 nM; “C”: >500 and <5000 nM; “D”: >=5000 nM; ND: Not determined

In vitro Assay: IC₅₀ Measurements for binding to ERα_LBD (GST)
Final assay conditions:
   1. ERα_LBD (GST) protein: 4 nM
   2. Tb Anti-GST: 2 nM
   3. Fluormone ES2 Green tracer: 3 nM
   4. Incubation time: 60 min
   5. DMSO: 1%
   6. Assay buffer: Adding 1M DTT to Nuclear receptor Buffer K for final 5 mM DTT.
   7. ZPE: 1% DMSO
   8. HPE: 1 μM ARV_471 (positive control)
   9. LanthaScreen® TR-FRET ERα Competitive Binding Assay (ThermoFisher, #A¹⁵⁸⁸⁷)
100× Compound preparation

1) Cherry pick 2 μL 10 mM compound stock to column 1 of a 384 intermediate plate.
2) Add 18 μL DMSO to column 1 to dilute compound to 1 mM.
3) Transfer 10 μL 1 mM compound to column 1 of a LDV plate.
2) Add 6 μl DMSO to column 2-10 of the LDV plate.
3) Compounds undergo 3-fold serial dilution (3 μL+6 μL) in DMSO.
4) Transfer 120 nL compound solution to assay plate. ZPE: 120 nL 100% DMSO
Procedure:

Complete nuclear receptor buffer K was prepared by adding 1 M DTT to nuclear receptor buffer K for a final concentration of 5 mM DTT. Complete nuclear receptor buffer K must be prepared fresh daily, 2X protein solution was prepared using complete nuclear receptor buffer K containing 8 nM ERα_LBD (GST) and 4 nM Tb Anti-GST. Then, 2X Fluormone ES2 Green tracer (6 nM) was prepared using complete nuclear receptor buffer K. Add 6 μL 2X Fluormone ES2 green tracer into a compound plate (PerkinElmer 6008289) by dragonfly with one-tips-addition. Subsequently, 6 μL 2× protein solution was added into the plate. The 384-well plate was mixed on a plate shaker and incubated at room temperature protected from light for 60 min. The plate was scaled with a cover to minimize evaporation. The plate was read at wavelengths of 520 nm and 495 nm. The TR-FRET ratio was calculated by dividing the emission signal at 520 nm by the emission signal at 495 nm. A binding curve was generated by plotting the emission ratio vs, the log [ligand]. To determine the IC₅₀ value, fit the data using XL-fit for a sigmoidal dose-response.

TABLE 9
ERα binding IC₅₀

| Compound No. | ERα HTRF IC₅₀ (nM) |
|---|---|
| A2 | A |
| A6 | A |
| A9 | B |
| A11 | B |
| A18 | C |
| A24 | A |
| A25 | A |
| A30 | A |
| A31 | A |
| A32 | C |
| A33 | A |
| A35 | A |
| A41 | A |
| A43 | A |
| A52 | A |
| A54 | A |
| A56 | A |
| A68 | A |
| A69 | A |
| A71 | A |
| A72 | A |
| A73 | A |
| A76 | A |
| A77 | A |
| A82 | A |
| A91 | A |
| A94 | A |
| A95 | A |
| A99 | A |
| A100 | A |
| A101 | A |
| A112 | A |
| A116 | A |
| A123 | A |
| A129 | A |
| A131 | A |
| A133 | A |
| A134 | A |
| A135 | A |
| A136 | A |
| A154 | A |
| A161 | A |
| A166 | A |
| A173 | A |
| A174 | A |
| A175 | A |
| A176 | A |

Note:
IC₅₀: “A”: <10 nM; “B”: 10-100 nM; “C”: >100 nM.

In-cell Western (ICW) assays in MCF-7 and T47D cell lines.

### Reagents and Consumables for ICW

1) MCF-7 from HDB
2) T47D from HDB
3)CS-FBS, BI, Cat #04-201-1
4) phenol red-free RPMI1640, Thermo, Cat #11835
5) P/S, Biosera Liquid, Cat #XC-A4122
6) 384-well cell plate (black), Corning, Cat #3764
7) PFA, Electron Microscopy Sciences, Cat #15710
8) Intercept (PBS) Blocking Buffer, Licor, Cat #927-70001
9) Triton X-100, Sigma, Cat #X-100
10) ER antibody, CST, Cat #13258
11) IRDye 800CW Goat anti-Rabbit IgG, LiCor, Cat #926-32211
12) CellTag 700 Stain, Licor, Cat #926-41090
13)Odyssey® DLx Imaging System, LiCor
14) En Vision, PerkinElmer.

### MCF-7 and T47D ICW Assays

Day 1: MCF-7 and T47D cell (From HDB) were seeded in 384-well black plate with phenol red-free RPMI1640+10% CS-FBS+1% P/S medium (1*10⁴ for MCF-7 and 1.5*10⁴ for T47D cells/well, 30 μl medium) for overnight at 37° C., 5% CO₂ incubator.

Day 2: Cells were treated at desired compound concentrations (0.02 to 300 nM) and DMSO as vehicle control for 16 hrs at 37° C. 5% CO₂ incubator.

Day 3: After 16 hrs of compounds treatment, cells were fixed by 4% PFA and permeabilized with elution buffer (0.1% Triton X-100 in 1% PBS Solution). Subsequently, cells were blocked with Intercept (PBS) Blocking Buffer (Li-COR, Odyssey Blocking Buffer), and were stained with ER (1:500, Cell signaling) primary antibody for overnight at cold room.

Day 4: Remove the buffer, add IRDye 800CW Goat anti-Rabbit IgG Secondary Antibody (1:2000) and CellTag 700 Stain (1:500) in Intercept (PBS) Blocking Buffer. Finally, cell plate is placed in incubator to dry. Image and signal were captured on Odyssey® DLx Imaging System. Data was further analyzed using XLfit using four parameters dose response curve to determine DC₅₀ and Dₘₐₓ.

### Data Analysis

Data are analyzed by image studio V5.2 and XLfit.

Half maximal degradation concentration values (DC₅₀) and maximal degradation percentage (Dₘₐₓ. %) of ER are summarized in Table 10.

TABLE 10
ER degradation by in-cell western (ICW) assays

| Compound | MCF7 DC₅₀ | MCF7 Dₘₐₓ | T47D DC₅₀ | T47D Dₘₐₓ |
|---|---|---|---|---|
| No. | (nM) | (%) | (nM) | (%) |
| A1 | | | B | C |
| A2 | A | A | A | A |
| A3 | A | C | | |
| A4 | B | B | A | B |
| A5 | A | B | A | C |
| A6 | A | B | A | B |
| A7 | A | B | A | B |
| A8 | A | A | A | A |
| A9 | B | B | B | B |
| A10 | A | A | A | A |
| A11 | C | B | B | B |
| A12 | A | A | A | A |
| A13 | B | B | B | B |
| A14 | A | B | A | B |
| A15 | C | C | C | C |
| A16 | B | B | A | B |
| A17 | B | C | B | C |
| A18 | A | B | A | B |
| A19 | B | B | B | B |
| A20 | A | B | A | B |
| A21 | C | B | B | B |
| A22 | A | B | A | C |
| A23 | C | C | C | B |
| A24 | A | A | A | A |
| A25 | A | A | A | A |
| A26 | C | C | C | C |
| A27 | C | C | D | D |
| A28 | C | C | C | C |
| A29 | C | C | C | C |
| A30 | A | B | A | A |
| A31 | A | B | A | A |
| A32 | C | B | C | B |
| A33 | A | B | A | B |
| A34 | A | A | A | A |
| A35 | A | A | A | A |
| A36 | A | B | A | C |
| A37 | A | C | A | C |
| A38 | C | C | B | C |
| A39 | C | C | B | B |
| A40 | C | C | C | C |
| A41 | A | A | A | A |
| A42 | C | B | C | B |
| A43 | A | B | A | A |
| A44 | D | D | D | D |
| A45 | D | D | D | D |
| A46 | C | C | C | C |
| A47 | C | B | B | C |
| A48 | B | B | B | B |
| A49 | A | B | A | B |
| A50 | A | B | A | B |
| A51 | D | D | D | D |
| A52 | A | B | A | A |
| A53 | C | C | B | B |
| A54 | A | B | A | A |
| A55 | C | C | B | B |
| A56 | A | B | A | A |
| A57 | C | C | B | B |
| A58 | A | A | A | B |
| A59 | A | D | D | D |
| A60 | D | C | D | D |
| A61 | D | D | D | D |
| A62 | C | C | B | C |
| A64 | B | B | B | B |
| A65 | B | B | A | A |
| A66 | B | B | B | B |
| A67 | B | B | B | B |
| A68 | A | B | A | A |
| A69 | A | B | A | A |
| A70 | A | B | ND | ND |
| A71 | A | A | ND | ND |
| A72 | A | B | A | A |
| A73 | A | A | A | A |
| A74 | A | B | ND | ND |
| A75 | A | B | ND | ND |
| A76 | A | A | ND | ND |
| A77 | A | B | A | A |
| A78 | A | B | ND | ND |
| A79 | A | B | ND | ND |
| A80 | A | B | ND | ND |
| A81 | A | C | ND | ND |
| A82 | A | A | ND | ND |
| A83 | ND | ND | A | B |
| A84 | A | D | C | C |
| A85 | A | C | A | B |
| A86 | B | D | C | C |
| A87 | A | C | A | C |
| A88 | B | B | A | C |
| A89 | A | C | A | C |
| A90 | A | C | A | C |
| A91 | A | A | A | A |
| A92 | A | C | B | C |
| A93 | A | B | A | B |
| A94 | A | A | A | A |
| A95 | A | A | A | A |
| A96 | A | B | A | B |
| A97 | ND | ND | B | A |
| A98 | ND | ND | C | C |
| A99 | A | A | A | A |
| A100 | A | A | A | A |
| A101 | A | A | ND | ND |
| A102 | ND | ND | B | A |
| A103 | ND | ND | C | B |
| A104 | B | B | A | A |
| A105 | A | B | B | B |
| A106 | A | B | A | B |
| A107 | A | B | A | B |
| A108 | A | B | A | B |
| A109 | A | C | A | B |
| A110 | A | B | A | B |
| A111 | B | B | B | A |
| A112 | A | A | ND | ND |
| A113 | C | C | C | C |
| A114 | ND | ND | D | D |
| A115 | ND | ND | B | A |
| A116 | A | B | A | A |
| A117 | C | C | ND | ND |
| A118 | A | C | ND | ND |
| A119 | A | C | A | C |
| A120 | C | C | ND | ND |
| A121 | B | A | ND | ND |
| A122 | A | B | A | B |
| A123 | A | A | A | A |
| A124 | C | B | C | B |
| A125 | ND | ND | C | B |
| A126 | A | B | A | A |
| A127 | B | B | ND | ND |
| A128 | B | B | ND | ND |
| A129 | A | A | A | A |
| A130 | B | A | ND | ND |
| A131 | A | A | ND | ND |
| A132 | B | B | ND | ND |
| A133 | A | A | ND | ND |
| A134 | A | A | ND | ND |
| A135 | A | A | A | 7A |
| A136 | A | A | A | A |
| A137 | B | D | ND | ND |
| A138 | A | B | ND | ND |
| A139 | B | B | ND | ND |
| A140 | A | A | B | A |
| A141 | B | B | ND | ND |
| A142 | A | B | ND | ND |
| A143 | C | B | ND | ND |
| A144 | B | B | ND | ND |
| A145 | C | B | ND | ND |
| A146 | C | C | ND | ND |
| A147 | A | A | B | A |
| A148 | A | B | A | A |
| A149 | B | A | ND | ND |
| A150 | C | B | ND | ND |
| A151 | C | C | ND | ND |
| A152 | B | B | ND | ND |
| A153 | D | D | ND | ND |
| A154 | A | B | A | A |
| A155 | B | C | C | D |
| A156 | A | A | ND | ND |
| A157 | C | D | ND | ND |
| A158 | A | B | ND | ND |
| A159 | C | C | ND | ND |
| A160 | C | B | ND | ND |
| A161 | A | B | ND | ND |
| A162 | A | C | ND | ND |
| A163 | B | A | ND | ND |
| A164 | B | A | ND | ND |
| A165 | C | B | ND | ND |
| A166 | A | B | ND | ND |
| A167 | A | B | ND | ND |
| A168 | B | B | ND | ND |
| A169 | C | C | ND | ND |
| A170 | C | C | ND | ND |
| A171 | A | A | ND | ND |
| A172 | C | C | ND | ND |
| A173 | A | A | A | A |
| A174 | A | A | A | A |
| A175 | A | A | A | A |
| A176 | A | A | A | A |
| A177 | A | A | | |
| A178 | A | A | | |
| A179 | A | A | | |
| A180 | A | A | | |
| A181 | A | A | | |
| A182 | A | B | | |
| A183 | A | B | | |
| A184 | A | B | | |
| A185 | A | A | | |
| A186 | A | B | | |
| A187 | A | A | | |
| A188 | A | A | | |
| A189 | A | A | | |
| A190 | A | B | | |
| A191 | A | B | | |
| A192 | A | A | | |
| A193 | A | A | | |
| A194 | B | A | | |
| A195 | A | A | | |
| A196 | A | A | | |
| A197 | A | A | | |
| A198 | A | B | | |
| A199 | A | B | | |
| A200 | A | A | | |
| A201 | A | B | | |
| A202 | A | B | | |
| A203 | A | A | | |
| A204 | B | B | | |
| A205 | B | C | | |
| A206 | B | B | | |
| A207 | A | A | | |
| A208 | | | | |
| A209 | | | | |
| A210 | A | A | | |
| A211 | | | | |
| A212 | | | | |
| A213 | | | | |
| A214 | | | | |
| A215 | | | | |
| A216 | | | | |
| A217 | | | | |
| A218 | A | A | | |
| A219 | | | | |
| A220 | | | | |
| A221 | | | | |
| A222 | A | A | | |
| A223 | | | | |
| A224 | | | | |
| A225 | | | | |
| A226 | A | A | | |
| A227 | A | A | | |
| A228 | | | | |
| A229 | A | A | | |
| A230 | A | A | | |
| A231 | A | A | | |
| A232 | A | A | | |
| A233 | A | A | | |
| A234 | A | A | | |
| A235 | A | A | | |
| A236 | A | A | | |

Note:
IC₅₀: “A”: <1 nM; “B”: 1-10 nM; “C”: >10 and <100 nM; “D”: >=100 nM.
Dₘₐₓ: “A”: >=75%; “B”: >50 and <75%; “C”: 25-50%; “D”: <25%.

### CellTiter-Glo® (CTG) Assays in MCF-7 and T47D Cell Lines.

### Reagents and Consumables for CTG

1) MCF-7 from HDB
2) T47D from HDB
3)CS-FBS, BI, Cat #04-201-1
4) phenol red-free RPMI1640, Thermo, Cat #11835
5) P/S, Biosera Liquid, Cat #XC-A4122
6) 384-well cell plate (white), Corning, Cat #3765
7) Cell TiterGlo reagent, Promega, Cat #G7573
8) En Vision, PerkinElmer.

### Medium

1) Cell culture medium: phenol red-free RPMI1640+10% CS-FBS,1% P/S

### Procedures for CTG Assay

In vitro Assay: MCF-7 and T47D CTG assay

Day-1: MCF-7 and T47D cell (From HDB) were cultured in 384-well white plate with phenol red-free RPMI1640+10% CS-FBS+1% P/S medium (1,000cells/well) for overnight at 37° C., 5% CO₂ incubator.

Day 0: Cells were treated at desired compound concentrations (0.5 to 10000 nM) (DMSO and Staurosporine as control) for Day 6 at 37° C.,5% CO₂ incubator.

Day 0 and Day 6: add Cell TiterGlo reagent and read on En Vision after 30 min incubation for data generation.

### Data Analysis

Data are analyzed by image studio V5.2 and XLfit.

Half maximal inhibitory concentration values (IC₅₀) of MCF-7 and T47D cell proliferation are summarized in Table 11.

TABLE 11
CellTiter-Glo ® (CTG) IC₅₀

| Compound No. | MCF7 IC₅₀ (nM) | T47D IC₅₀ (nM) |
|---|---|---|
| A12 | B | |
| A24 | B | |
| A25 | A | |
| A31 | B | C |
| A34 | A | A |
| A35 | B | B |
| A41 | A | A |
| A43 | B | B |
| A65 | B | |
| A68 | B | |
| A71 | A | |
| A72 | A | |
| A73 | B | |
| A76 | A | |
| A77 | A | |
| A100 | A | A |
| A135 | A | A |
| A136 | A | A |
| A173 | A | A |
| A174 | A | A |
| A175 | A | A |
| A176 | A | A |
| A177 | C | |
| A178 | C | |
| A179 | C | |
| A180 | C | |
| A181 | C | |
| A184 | C | |
| A185 | C | |
| A187 | B | |
| A188 | B | |
| A189 | B | |
| A191 | B | |
| A192 | C | |
| A193 | B | |
| A194 | C | |
| A195 | C | |
| A196 | A | |
| A197 | B | |
| A198 | B | |
| A199 | A | |
| A200 | A | |
| A201 | C | |
| A202 | C | |
| A203 | A | |
| A204 | C | |
| A205 | C | |
| A206 | C | |
| A207 | C | |
| A208 | B | |
| A209 | A | |
| A210 | A | |
| A213 | A | |
| A214 | A | |
| A215 | A | |
| A217 | A | |
| A218 | A | |
| A219 | A | |
| A220 | A | |
| A221 | C | |
| A222 | A | |
| A223 | A | |
| A224 | B | |
| A225 | A | |
| A226 | A | |
| A227 | A | |
| A228 | B | |
| A229 | B | |
| A230 | A | |
| A231 | A | |
| A232 | A | |
| A233 | A | |
| A234 | A | |
| A235 | A | |
| A236 | B | |

Note:
IC₅₀: “A”: <10 nM; “B”: 10-100 nM; “C”: >100 nM.

### For “B” Compounds

In-cell western blot analysis, a, seed cells in black-sided/clear bottom 96- or 384-well plates at 40,000 or 10,000 cells/well, overnight; b, add diluted compounds (final 0.5% DMSO), 16 hours, 16 h later, remove medium, add 100 L or 25 L of 3.7-4.0% formaldehyde (PBS:FA=9:1), R^{T} 20 min, no shaking; c, wash with PBS, and permeabilized with 100 μL or 25 μL/well of 1×PBS+0.1% Triton X-100 10 minutes; d, block with 100 μL or 25 μL Licor blocking buffer (Li-Cor), R^{T} 1h, moderate shaking; d. Add 100 μL or 25 μL of anti-ER (cs-8644, 1:500-1.000)+GAPDH (Millipore MAB374, 1:1000) in Block+0.05% Tween 20. R^{T} 2h, gentle shaking. Negative control: cells plus secondary antibodies (no primary antibodies); e, wash x 4 with PBS+0.05-0.1% Tween 20, gentel shaking; f, anti-rabbit-680 and anti-mouse-800 (both 1:1000 in LiCor block+0.05% Tween20, R^{T} 1h, gentle shaking, no light. LI-COR: 0.2% to reduce background; g, wash×4 with PBS+0.05% Tween 20, gental shaking; h, add 100 μL or 25 μL of PBS to each well and read on CLX plate reader. The relative ER percentage in treated cells were obtained by comparing the values of treated wells to those in untreated and DMSO-treated wells as 100%.

Western Blot Analysis. Western blot analysis was performed essentially as described previously. The cells treated with indicated compounds were lysed in Radioimmunoprecipitation Assay Protein Lysis and Extraction Buffer (25 mmol/I. Tris.HCl, pH 7.6, 150 mmol/L, NaCl, 1% Nonidet P-40, 1% sodium deoxycholate, and 0.1% sodium dodecyl sulfate) containing proteinase inhibitor cocktail (Roche Diagnostics, Mannheim, Germany). Equal amounts of total protein were electrophoresed through 10% SDS-polyacrylamide gels after determination of protein concentration by BCA assay (Fisher Scientific, Pittsburgh, PA), The separated protein bands were transferred onto PVDF membranes (GE Healthcare Life Sciences, Marlborough, MA) and blotted against different antibodies, as indicated. The blots were scanned, and the band intensities were quantified using GelQuant.NET software provided by biochemlabsolutions.com. The relative mean intensity of target proteins was expressed after normalization to the intensity of glyceraldehyde-3-phosphate dehydrogenase bands.

Cell Growth Assay. The cells were seeded at 1500/well in 96 well plates overnight. One day after the seeding, they were treated with indicated doses of compounds respectively, 4 days after the compound treatment, 10% WST-8 reagent was added to the culture medium and incubate in a CO₂ incubator at 37° C. for 2.5 hours. Before reading, the plate was mixed gently on an orbital shaker for one minute to ensure homogeneous distribution of color. The absorbance was measured of each sample using a microplate reader at a wavelength of 450 nm. The relative absorbance was calculated against the vehicle control from three individually repeats.

In vivo pharmacodynamic and efficacy studies. To develop breast cancer cell line xenografts, mice was given 4 ug/ml 17ß-Estradiol in 0.05% ETOH dringking water for 1 week, followed with 8 ug/ml 17B-Estradiol in 0.1% EtOH drinking water thereafter. Five million cells in 50% Matrigel were injected subcutaneously into SCID mice, when tumors reached 100-400 mm³, mice were treated with vehicle control (5% DMSO, 10% solutol, 85% Water) or indicated dose of the drugs, sacrificed at indicated time-points, and tumor tissue was harvested for analysis. For in vivo efficacy experiments, when tumors reached 80-200 mm³, mice were randomized into groups. vehicle control (5% DMSO, 10% solutol, 85% Water) was given at the dose and with the duration indicated. Tumor sizes and animal weights were measured 2-3 times per week. Tumor volume (mm³)=(length×width2)/2. Tumor growth inhibition was calculated as TGI (%)= (Vc−Vt)/(Vc−Vo)×100, where Vc. Vt are the median of control and treated groups at the end of the study and Vo at the start. All the in vivo studies were performed under an animal protocol (PRO00005315) approved by the University Committee on Use and Care of Animals of the University of Michigan, in accordance with the recommendations in the Guide for the Care and Use of Laboratory Animals of the National Institutes of Health.

TABLE 12
Biological Data for Compounds B1 to B401

| | | | Cell growth |
|---|---|---|---|
| | Traditional Western | | Inhibition in |
| Compound | Degradation Potency | ICW DC₅₀ | T47D cell line |
| No. | (DC₅₀) | (nM) | IC₅₀ (nM) |
| B1 | C | | |
| B2 | B | | |
| B3 | A | | |
| B4 | B | | |
| B5 | A | | C |
| B6 | A | | |
| B7 | A | | |
| B8 | B | | |
| B9 | B | | |
| B10 | B | | |
| B11 | A | | |
| B12 | B | | C |
| B13 | C | | |
| B14 | C | | |
| B15 | B | | |
| B16 | B | | |
| B17 | B | | |
| B18 | B | | |
| B19 | B | | |
| B20 | B | | |
| B21 | C | | |
| B22 | C | | |
| B23 | B | | C |
| B24 | B | | C |
| B25 | C | | |
| B26 | B | | |
| B27 | C | | |
| B28 | A | | B |
| B29 | C | | |
| B30 | C | | C |
| B31 | C | | |
| B32 | C | | |
| B33 | C | | |
| B34 | C | | |
| B35 | B | | |
| B36 | C | | |
| B37 | C | | |
| B38 | C | | |
| B39 | C | | |
| B40 | C | | |
| B41 | C | | |
| B42 | C | | |
| B43 | C | | B |
| B44 | B | | C |
| B45 | C | | C |
| B46 | C | | C |
| B47 | B | | B |
| B48 | B | | B |
| B49 | B | | B |
| B50 | B | | C |
| B51 | C | | NA |
| B52 | C | | B |
| B53 | C | | |
| B54 | C | | |
| B55 | B | | |
| B56 | B | | B |
| B57 | C | | B |
| B58 | C | | |
| B59 | C | | |
| B60 | C | | |
| B61 | A | | |
| B62 | B | | |
| B63 | C | | |
| B64 | A | | B |
| B65 | C | | |
| B66 | B | | |
| B67 | C | | |
| B68 | A | | |
| B69 | C | | |
| B70 | C | | |
| B71 | C | | |
| B72 | B | | |
| B73 | C | | |
| B74 | C | | |
| B75 | C | | |
| B76 | B | | |
| B77 | C | | |
| B78 | C | | |
| B79 | C | | |
| B80 | C | | |
| B81 | C | | |
| B82 | C | | |
| B83 | B | | |
| B84 | C | | |
| B85 | C | | |
| B86 | C | | |
| B87 | C | | |
| B88 | C | | |
| B89 | C | | |
| B90 | C | | |
| B91 | B | | |
| B92 | C | | |
| B93 | C | | |
| B94 | C | | |
| B95 | C | | |
| B96 | C | | |
| B97 | C | | |
| B98 | C | | |
| B99 | C | | |
| B100 | C | | |
| B101 | C | | |
| B102 | C | | |
| B103 | C | | |
| B104 | C | | |
| B105 | A | | |
| B106 | A | | |
| B107 | C | | |
| B108 | B | | |
| B109 | A | | |
| B110 | C | | |
| B111 | C | | |
| B112 | A | | |
| B113 | B | | |
| B114 | C | | |
| B115 | C | | |
| B116 | C | | |
| B117 | C | | |
| B118 | C | | |
| B119 | C | | |
| B120 | C | | |
| B121 | B | | |
| B122 | B | | |
| B123 | A | | |
| 1B24 | C | | |
| B125 | A | | |
| B126 | B | | |
| B127 | C | | |
| B128 | B | | |
| B129 | C | | |
| B130 | C | | |
| B131 | C | | |
| B132 | C | | |
| B133 | B | | B |
| B134 | C | | |
| B135 | B | | B |
| B136 | B | | B |
| B137 | B | | |
| B138 | C | | B |
| B139 | B | | |
| B140 | | A | A |
| B141 | | A | A |
| B142 | | A | A |
| B143 | | A | A |
| B144 | | B | A |
| B145 | | B | A |
| B146 | | A | A |
| B147 | | B | B |
| B148 | | B | B |
| B149 | | B | A |
| B150 | | A | A |
| B151 | | A | A |
| B152 | | A | A |
| B153 | | A | A |
| B154 | | A | A |
| B155 | | A | A |
| B156 | | C | |
| B157 | | B | A |
| B158 | | B | |
| B159 | | B | B |
| B160 | | A | |
| B161 | | A | |
| B162 | | B | |
| B163 | | A | B |
| B164 | | A | B |
| B165 | | C | A |
| B166 | | A | A |
| B167 | | A | A |
| B168 | | A | A |
| B169 | | A | |
| B170 | | A | A |
| B171 | | A | A |
| B172 | | B | A |
| B173 | | C | A |
| B174 | | C | A |
| B175 | | B | A |
| B176 | | B | B |
| B177 | | C | B |
| B178 | | C | A |
| B179 | C | | |
| B180 | C | | |
| B181 | C | | |
| B182 | B | | |
| B183 | A | | |
| B184 | A | | |
| B185 | B | | |
| B186 | B | | |
| B187 | B | | |
| B188 | B | | |
| B189 | C | | |
| B190 | C | | |
| B191 | B | | |
| B192 | B | | |
| B193 | B | | |
| B194 | B | | |
| B195 | B | | |
| B196 | B | | |
| B197 | B | | |
| B198 | B | | |
| B199 | C | | |
| B200 | B | | |
| B201 | B | | |
| B202 | C | | |
| B203 | | A | A |
| B204 | | A | A |
| B205 | | A | B |
| B206 | | A | A |
| B207 | | A | A |
| B208 | | A | A |
| B209 | | A | A |
| B210 | | A | A |
| B211 | | A | B |
| B212 | | A | A |
| B213 | | A | A |
| B214 | | A | A |
| B215 | | B | B |
| B216 | | B | C |
| B217 | | C | B |
| B218 | | C | A |
| B219 | | B | A |
| B220 | | C | A |
| B221 | | B | A |
| B222 | | B | A |
| B223 | | B | C |
| B224 | | C | B |
| B225 | | B | B |
| B226 | | A | A |
| B227 | | A | A |
| B228 | | A | B |
| B229 | | B | B |
| B230 | | A | A |
| B231 | | B | A |
| B232 | | C | B |
| B233 | | C | B |
| B234 | | C | A |
| B235 | | C | B |
| B236 | | B | A |
| B237 | | C | B |
| B238 | | A | A |
| B239 | | B | B |
| B240 | | A | B |
| B242 | | A | A |
| B243 | | B | B |
| B244 | | B | A |
| B245 | | B | A |
| B246 | | A | A |
| B247 | | B | A |
| B248 | | B | A |
| B249 | | B | A |
| B250 | | B | A |
| B251 | | A | A |
| B252 | | A | A |
| B253 | | A | A |
| B254 | | B | A |
| B255 | | B | B |
| B256 | | B | B |
| B257 | | C | B |
| B258 | | B | A |
| B259 | B | | |
| B260 | C | | |
| B261 | C | | |
| B262 | C | | |
| B263 | C | | |
| B264 | C | | |
| B265 | C | | |
| B266 | C | | |
| B267 | C | | |
| B268 | C | | |
| B269 | C | | |
| B270 | C | | |
| B271 | C | | |
| B272 | B | | |
| B273 | C | | |
| B274 | C | | |
| B275 | C | | |
| B276 | C | | |
| B277 | C | | |
| B278 | C | | |
| B279 | C | | |
| B280 | C | | |
| B281 | B | | |
| B282 | C | | |
| B283 | C | | |
| B284 | C | | |
| B285 | B | | |
| B286 | C | | |
| B287 | C | | |
| B288 | C | | |
| B289 | C | | |
| B290 | C | | |
| B291 | C | | |
| B292 | C | | |
| B293 | B | | |
| B294 | A | | |
| B295 | C | | |
| B296 | C | | |
| B297 | C | | |
| B298 | B | | |
| B299 | C | | |
| B300 | C | | |
| B301 | C | | |
| B302 | C | | |
| B303 | C | | |
| B304 | C | | |
| B305 | C | | |
| B306 | | C | A |
| B307 | | C | B |
| B308 | | A | A |
| B309 | | A | |
| B310 | | C | A |
| B311 | | C | A |
| B312 | | C | A |
| B313 | | B | A |
| B314 | | C | B |
| B315 | | C | B |
| B316 | | B | B |
| B317 | | C | B |
| B318 | | C | |
| B319 | | C | |
| B320 | | C | |
| B321 | | C | |
| B322 | | A | |
| B323 | C | | |
| B324 | C | | |
| B325 | | B | B |
| B326 | | C | B |
| B327 | | B | A |
| B328 | | B | B |
| B329 | | C | C |
| B330 | | A | A |
| B331 | | C | |
| B332 | | A | B |
| B333 | | A | A |
| B334 | | B | A |
| B335 | | B | A |
| B336 | | B | B |
| B337 | | A | A |
| B338 | | B | B |
| B339 | | C | B |
| B340 | | B | B |
| B341 | | A | A |
| B342 | | A | B |
| B343 | | C | B |
| B344 | | C | B |
| B345 | | C | B |
| B346 | | B | B |
| B347 | | B | A |
| B348 | | A | |
| B349 | | B | |
| B350 | | B | C |
| B351 | | A | B |
| B352 | | B | B |
| B353 | | B | |
| B354 | | B | |
| B355 | | B | A |
| B356 | | A | B |
| B358 | | B | C |
| B359 | | B | B |
| B360 | | C | C |
| B362 | | B | C |
| B363 | | B | C |
| B364 | | C | B |
| B365 | | B | C |
| B366 | | A | A |
| B367 | | C | B |
| B368 | | C | B |
| B369 | | B | A |
| B370 | | C | B |
| B371 | | A | A |
| B372 | | B | B |
| B373 | | A | A |
| B374 | | A | A |
| B375 | | B | A |
| B376 | | B | A |
| B377 | | B | A |
| B378 | | C | |
| B379 | | C | |
| B380 | | C | |
| B381 | | C | |
| B382 | | C | |
| B383 | | C | C |
| B384 | | C | B |
| B385 | | C | B |
| B386 | | C | B |
| B387 | | C | B |
| B388 | | | A |
| B389 | | C | C |
| B390 | | B | C |
| B391 | | C | B |
| B392 | | | B |
| B393 | | | A |
| B394 | | | B |
| B395 | | | B |
| B396 | | A | A |
| B397 | | A | A |
| B398 | | A | A |
| B399 | | A | A |
| B400 | | A | A |
| B401 | | A | A |

Note:
DC₅₀: “A”: <10 nM; “B”: 10-100 nM; “C”: >100 nM.
IC₅₀: “A”: <1 nM; “B”: 1-10 nM; “C”: >10 nM.

### In Vitro Efficacy Studies

### ER Degradation in Breast Cancer Cell Lines

ER degradation is measured using several different breast cancer cell lines in multiple cellular assays. Cell lines to be used for this purpose include, but are not limited to, MCF-7 cells (ATCC, catalog #HB-22), T47D cells (ATCC, catalog #HTB-133), or CAMA1 cells (ATCC, catalog #HTB-21) expressing wild type ER, or breast cancer cell lines expressing clinically relevant ER gene mutations, such as MCF-7 cells engineered to express Q380E. Y537S, or D538G ER. Endogenous ER in breast cancer cell lines is measured using Western blot, in-cell Western assay or HiBiT assay in cells engineered to express a HiBiT-tagged version of ER. ER degradation is measured at times, e.g., between 2 and 24 hours. Cells are treated with vehicle control (DMSO) or the compound at various concentrations (e.g., ranging from 0.005 nM to 100 nM). Some assays are conducted in the presence of estradiol, while other assays are conducted in the absence of estradiol. The compounds of this disclosure are expected to degrade ER protein in breast cancer cell lines.

### Cell Growth Inhibition in Breast Cancer Cell Lines

Cell growth inhibition is measured using several different cell lines (e.g., the ones mentioned above) to test whether ER degradation with the compounds of this disclosure impacts cell growth inhibition in breast cancer cell lines. Cells are treated with vehicle control (DMSO) or the compound at various concentrations (e.g., ranging from 0.003 nM to 100 nM) for about 144 hours. Briefly cells per well are plated in each well of a 384-well plate, 24 hours later, the compound is dispensed into the wells, 144 hours after compound is added to wells, CellTiter-Glo (Promega) is added to wells and plates are read on an EnVision® Plate Reader (Perkin Elmer). The compounds of this disclosure are expected to inhibit or retard cell growth in breast cancer cell lines.

### In Vivo Pharmacokinetic and Pharmacodynamic (PKPD) and Efficacy Studies

### ER Degradation in MCF-7 Tumor Model

To evaluate the ability of compounds of this disclosure to reduce ER protein levels in vivo, an orthotopic human breast cancer MCF7 xenograft model in female NOD/SCID mice is used. Each mouse is implanted subcutaneously with estrogen pellets at the right flank before the tumor inoculation. Each mouse is inoculated at the right third mammary fat pad with MCF7 tumor cells (2×10⁷) in 0.2 mL of PBS with Matrigel (1:1) for tumor development. Mice are treated with vehicle control (e.g., 5% DMSO, 10% solutol, 85% water) or the compound for 6 or 24 hours past the 3^{rd} dose once tumors reach 400-500 mm³. Tumors arc harvested at given times, bisected and flash frozen. Half of the tumor is analyzed for compound concentration in the tumor or plasma and the other half is analyzed using Western blot to quantify the extent of ER degradation. The compounds of this disclosure are expected to demonstrate dose-dependent ER degradation in MCF-7 tumor model.

### Tumor Growth Inhibition and Regression in Mice

To evaluate the ability of compounds of this disclosure to inhibit tumor growth and/or cause tumor growth regression in vivo, the MCF-7 human breast carcinoma female athymic nude mouse model is used. Mice are supplemented with 10 μg/mL 17 beta-estradiol in their drinking water 3 days prior to cell implantation and then for the duration of the study. Mice are injected with 1×10⁷ MCF-7 tumor cells in PBS subcutaneously in the flank. Mice are treated with vehicle control (e.g., 5% DMSO, 10% solutol, 85% Water) or the compound once tumors reach 150-200 mm³, and sacrificed when tumor volume reaches 2000 mm³ or at the end of the study (whichever occurs first). Tumor sizes and animal weights and caliper measurements of tumors are collected 2-3 times per week. Tumor volume (mm³)=(lengthxwidth2)/2. Tumor growth inhibition is calculated using ^{D}T/^{D}C TGI (%)=(1-((Tₑ−T₀)/(Cₑ−C₀)))*100, where ^{D}T/^{D}C is the difference (delta) or change in test vs control TGI; Tₑ-Test tumor volume endpoint, T₀=Test tumor volume at start of dosing, Cₑ=Vehicle control tumor volume endpoint, C₀=Vehicle control tumor volume at start of dosing. Tumor growth regression is calculated using % Tumor Regression=−(1−(Te/T0))*100) where Te=Test tumor volume (TV) endpoint, Test T0=TV at start of dosing. The compounds of this disclosure are expected to inhibit tumor growth and induce tumor shrinkage over a range of doses.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula I:
T-L-C(I,
or a pharmaceutically acceptable salt, solvate, or stereoisomer thereof,
wherein:
C is of Formula I′-1 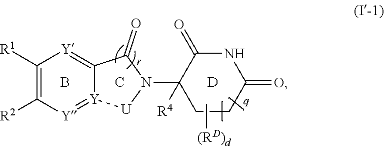
wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(-O) R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R² is *—Cy²—, wherein * denotes attachment to L;
—Cy²- is C₃₋₁₂ carbocyclylene or 3- to 12-membered heterocyclylene, wherein the carbocyclylene or heterocyclylene is optionally substituted with one or more R^{u}; or
R¹ and R², together with the intervening carbon atoms, form Ring A attached to L, wherein Ring A is optionally substituted C₃₋₁₂ carbocycle or 5- to 16-membered heterocycle;
Y″ is N or CR³;
R³ is hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O) R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form Ring A attached to L, wherein Ring A is optionally substituted 5- to 16-membered heterocycle;
provided that R¹ and R², and R² and R³, do not both form Ring A attached to L;
Y′ is N or CR^{Y};
R^{Y} is hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}; [symbol]
denotes an optional covalent bond between Y and U;
i) when the bond between Y and U is absent:
r is 0 or 1;
Y is N or CR^{Y};
R^{Y} is hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
U is hydrogen or C₁₋₆ alkyl optionally substituted with one or more R^{u};
ii) when the bond between Y and U is present:
r is 1;
Y is C;
U is -CH₂-, —C(═O)—, —(C═O)-N(R^{U})-*, or —N═C(R^{U})-*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl;
each R^{D} is independently oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
d is an integer from 0 to 4; and
q is an integer from 0 to 2, T is of Formula I-2: 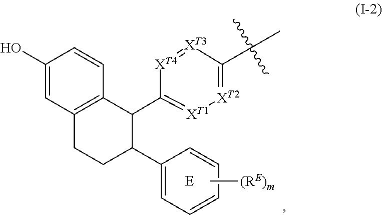
wherein:
each of X^{T1}, X^{T2}, X^{T3}, and X^{T4} is independently N or CRT;
each occurrence of R^{T} is independently hydrogen, halogen, -CN, —NO₂, —OH, —NH₂, C₁₋₆ alkyl, C₁₋₆-alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O) R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NRC(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{E} is independently halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O) R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
m is an integer selected from 0 to 5;
L is of Formula I′-3: 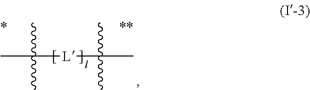
wherein:
* denotes attachment to T, and ** denotes attachment to C;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O) N (R^{L})—, —C(═O)O—, —N(R^{L})—, —O—, —S—, or —S(═O)₂—, wherein the alkylene, heteroalkylene, alkenylene, alkynylene, carbocyclylene, heterocyclylene, arylene, or heteroarylene is optionally substituted with one or more R^{u};
each occurrence of R^{L} is independently hydrogen, C₁₋₆ alkyl, C₂₋₆ alkonyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O): OR^{b}, —S(═O)₂NR^{c}R^{d}, —C(═O) R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
1 is an integer selected from 0 to 10,
wherein:
each R^{u} is independently oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O) R^{a}, —S(═O)₂R², —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R²; and
each R₂ is independently oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

2. The compound of claim 1, wherein
1) when the bond between Y and U is present, U is -CH₂-or-C(═O)—, and r is 1, then;
i) either R¹ and R², or R² and R³, together with the intervening carbon atoms, form Ring A attached to L; and
ii) Ring A is not 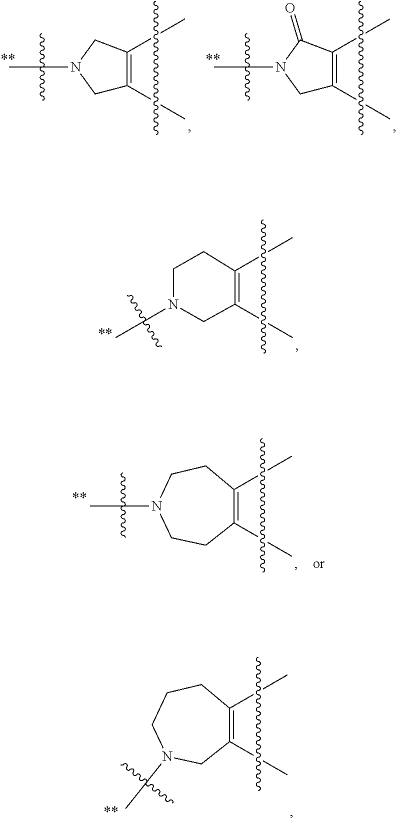
wherein ** denotes attachment to L; and
2, the compound is not 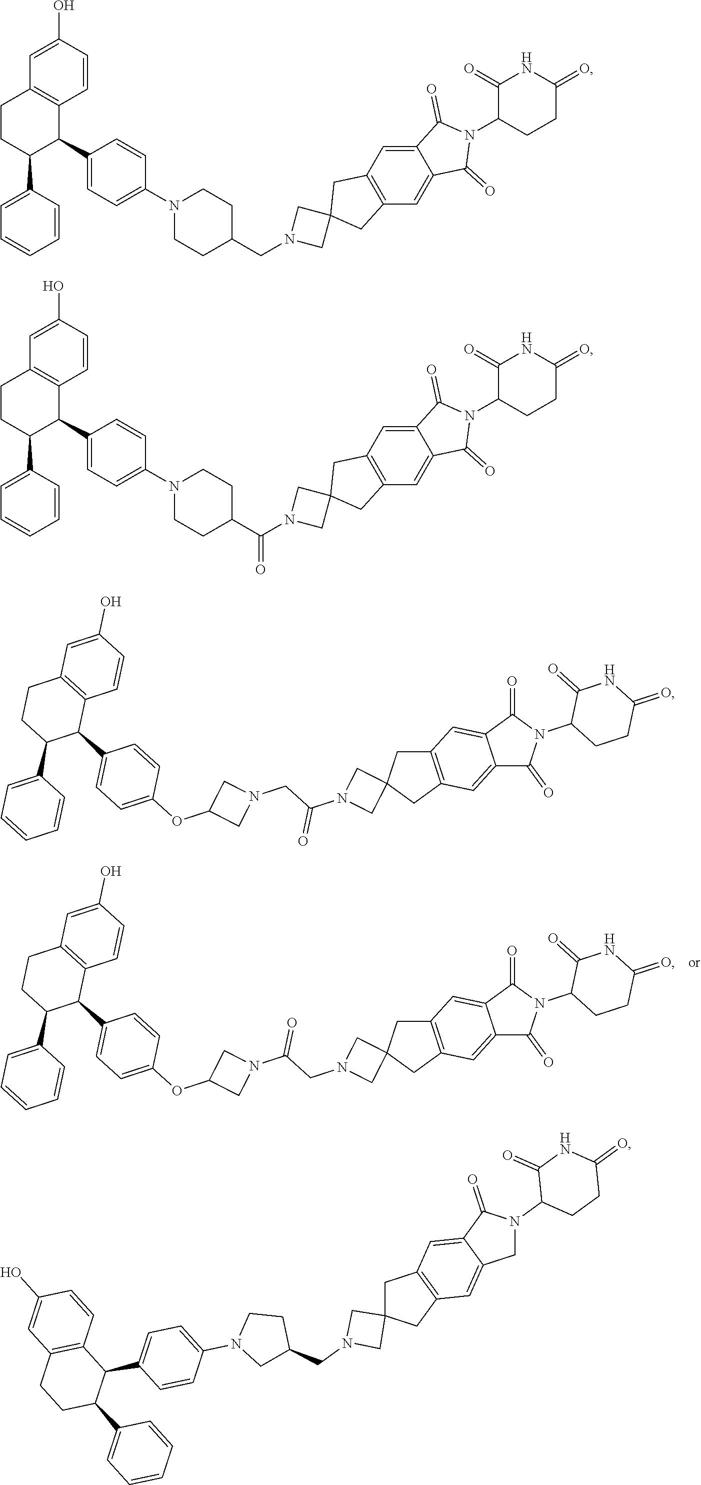
or a pharmaceutically acceptable salt, solvate, or stereoisomer thereof.

3. The compound of claim 1 or 2, wherein C is of Formula I′-1-i

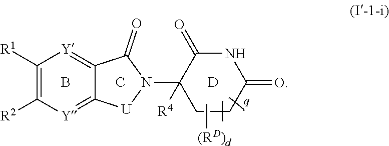

4. The compound of claim 3, wherein U is -CH₂-or-C(═O)—.

5. The compound of claim 1 or 2, wherein C is of Formula P′-1-i

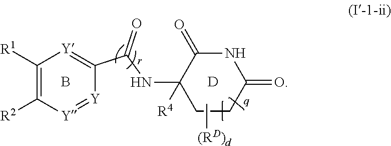

6. The compound of claim 5, wherein Y is N.

7. The compound of claim 5, wherein Y is CR^{Y}, and R^{Y} is hydrogen, halogen, or C₁₋₆ alkoxy.

8. The compound of any one of claims 1-7, wherein R¹ and R², together with the intervening carbon atoms, form Ring A attached to L, wherein the Ring A is optionally substituted 5- to 16-membered heterocycle.

9. The compound of claim 8, wherein Y″ is N.

10. The compound of claim 8, wherein Y″ is CR³, and R³ is hydrogen, halogen, -CN, —NO₂, —OH, -NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

11. The compound of claim 10, wherein R³ is hydrogen, halogen, or C₁₋₆ alkoxy.

12. The compound of any one of claims 1-7, wherein R² and R³, together with the intervening carbon atoms, form Ring A attached to L, wherein the Ring A is optionally substituted 5- to 16-membered heterocycle.

13. The compound of claim 12, wherein R¹ is hydrogen, halogen, -CN, —NO₂, —OH, —NH₂, C₁₋₆-alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

14. The compound of claim 13, wherein R¹ is hydrogen, halogen, or C₁₋₆ alkoxy.

15. The compound of any one of claims 1-14, wherein Ring A is optionally substituted 7- to 16-membered fused heterocycle.

16. The compound of any one of claims 1-14, wherein Ring A is 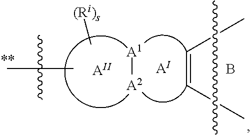
wherein:
** denotes attachment to L;
Ring A^{I} and Ring A^{II} are independently C_{4 8} carbocycle or 4- to 8-membered heterocycle; wherein at least one of Ring A^{III} and Ring A^{IV} is 4- to 8-membered heterocycle;
A¹ and A² are independently C, CR^{Ax}, or N;
R^{Ax} is hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
each Rⁱ is independently oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O) R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S (═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits,
wherein each Rⁱ may independently be present on either Ring A^{I} or Ring A^{II}.

17. The compound of any one of claims 1-14, wherein Ring A is 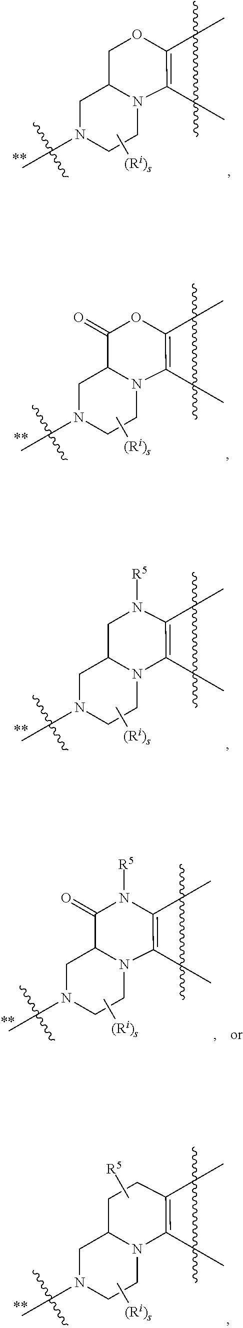
wherein:
** denotes attachment to L;
R⁵ is hydrogen or C₁₋₆ alkyl optionally substituted with one or more R^{u};
each Rⁱ is independently oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O) R^{a}, —S(-O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

18. The compound of any one of claims 1-14, wherein Ring A is optionally substituted 7- to 16-membered spiro heterocycle.

19. The compound of any one of claims 1-14, wherein Ring A is: 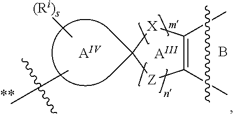
wherein:
** denotes attachment to L;
Ring A^{IV} is C₃₋₈ carbocycle or 3- to 8-membered heterocycle;
each X is independently —C(R^{X1})₂-, —NR^{X2}-, —O—, —S—, —S(═O)—, or —S(═O)₂-;
each Z is independently —C(R^{Z1})₂-, —NR^{Z2}-, —O—, —S—, —S(═O)—, or —S(═O)₂-;
each occurrence of R^{X1} and RZ1 is independently hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O) R^{a}, —S(═O)₂R^{a}, —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
two geminal R^{X1} or two geminal RZ1 together form oxo;
each occurrence of R^{X2} and R^{Z2} is independently hydrogen or C_{1 6} alkyl optionally substituted with one or more R^{u};
m′ and n′ are independently an integer selected from 0 to 3, wherein m′ and n′ are not both 0;
s is an integer selected from 0 to 8, as valency permits, and
each Rⁱ is independently oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O) R^{a}, —S(═O)₂R², —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S (═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
provided that when none of m′ and n′ is 0, then Ring A^{I} is 4- to 9-membered heterocycle, and
each Rⁱ may independently be present on either Ring A^{III} or Ring A^{IV}.

20. The compound of any one of claims 1-14, wherein Ring A is: 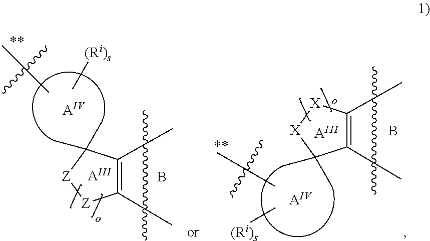
wherein o is 0 or 1; or 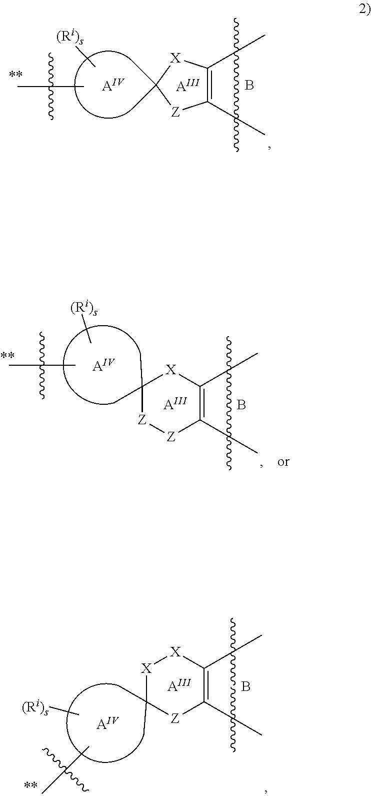
wherein ** denotes attachment to L.

21. The compound of any one of claims 1-14, wherein Ring A is optionally substituted 5- to 6-membered heterocycle.

22. The compound of any one of claims 1-14, wherein Ring A is 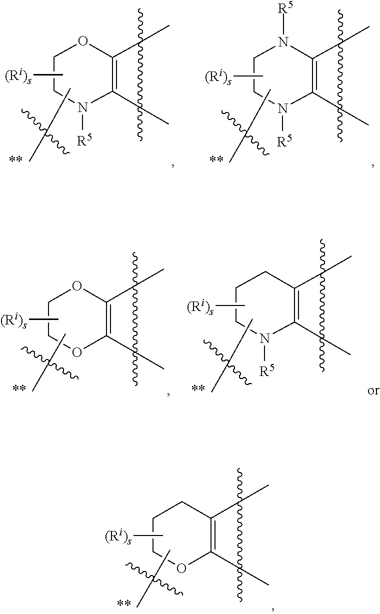
wherein:
** denotes attachment to L;
R⁵ is hydrogen or C₁₋₆ alkyl optionally substituted with one or more R^{u};
each Rⁱ is independently oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O) R^{a}, —S(═O)₂R², —S(═O)₂OR^{b}, —S(═O)₂NR^{c}R^{d}, —NR^{c}S(═O)₂R^{a}, —NR^{c}S(═O) R^{a}, —NR^{c}S(═O)₂OR^{b}, —NR^{c}S(═O)₂NR^{c}R^{d}, —NR^{b}C(═O)NR^{c}R^{d}, —NR^{b}C(═O) R^{a}, —NR^{b}C(═O)OR^{b}, —OS(═O)₂R^{a}, —OS(═O)₂OR^{b}, —OS(═O)₂NR^{c}R^{d}, —OC(═O) R^{a}, —OC(═O)OR^{b}, —OC(═O)NR^{c}R^{d}, —C(═O) R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

23. The compound of any one of claims 16-17, 19-20, and 22, wherein each Rⁱ is independently oxo, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

24. The compound of claim 23, wherein s is 0.

25. The compound of claim 1, wherein C is of Formula I′-1-ii

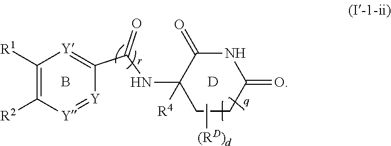

26. The compound of claim 25, wherein R² is *—Cy²—, wherein * denotes attachment to L.

27. The compound of claim 26, wherein—Cy²- is C₅₋₁₂ fused carbocyclylene or 5- to 12-membered fused heterocyclylene, wherein the carbocyclylene or heterocyclylene is optionally substituted with one or more R^{u}.

28. The compound of any one of claims 25-27, wherein R¹ is hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

29. The compound of claim 28, wherein R¹ is hydrogen, halogen, or C₁₋₆ alkoxy.

30. The compound of any one of claims 25-29, wherein Y is N.

31. The compound of any one of claims 25-29, wherein Y is CR^{Y}, and R^{Y} is hydrogen, halogen, or C₁₋₆ alkoxy.

32. The compound of any one of claims 25-31, wherein Y″ is CR³, and R³ is hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

33. The compound of claim 32, wherein R³ is hydrogen, halogen, or C₁₋₆ alkoxy.

34. The compound of any one of claims 1-33, wherein Y′ is CR^{Y}, and R^{Y} is hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

35. The compound of claim 34, wherein Y′ is CR^{Y}, and R^{Y}′ is hydrogen, halogen, or C₁₋₆ alkoxy.

36. The compound of any one of claims 1-35, wherein R⁴ is hydrogen.

37. The compound of any one of claims 1-36, wherein q is 1.

38. The compound of any one of claims 1-37, wherein each of X^{T1}, X^{T2}, X^{T3}, and X^{T4} is CRT.

39. The compound of claim 38, wherein each of X^{T1}, X^{T2}, X^{T3}, and X^{T4} is CH.

40. The compound of claim 38, wherein X^{T1} is C(OCH₃), X^{T3} is CF, and each of X^{T2} and X^{T4} is CH; X^{T2} is CF, X^{T4} is C(OCH₃), and each of XTI and X^{T3} is CH; one of X^{T1} and X^{T4} is CF or C(OCH₃), the other one of X^{T1} and X^{T4} is CH, and each of X^{T2} and X^{T3} is CH, or X^{T1} is C(OCH₃), X^{T2} is CF, and each of X^{T3} and X^{T4} is CH.

41. The compound of any one of claims 1-37, wherein one of X^{T1}, X^{T2}, X^{T3}, and X^{T4} is N.

42. The compound of claim 41, wherein one of XTI and X^{T4} is N, the other one of XTI and X^{T4} is CH, and each of X^{T2} and X^{T3} is CH; or one of X^{T2} and X^{T3} is N, the other one of X^{T2} and X^{T3} is CH, and each of X^{T1} and X^{T4} is CH.

43. The compound of any one of claims 1-37, wherein two of X^{T1}, X^{T2}, X^{T3}, and X^{T4} are N.

44. The compound of claim 43, wherein each of XTI and X^{T4} is CH, and each of X^{T2} and X^{T3} is N.

45. The compound of any one of claim 38, 41, or 43, wherein each R^{T} is independently hydrogen, halogen, -CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

46. The compound of claim 45, wherein each R^{T} is independently hydrogen or halogen.

47. The compound of any one of claims 38-46, wherein each R^{E} is independently halogen, -CN, -NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, or 3- to 6-membered heterocyclyl, wherein the alkyl, alkoxy, alkylamino, alkenyl, alkynyl, aryl, heteroaryl, carbocyclyl, or heterocyclyl is optionally substituted with one or more R^{u}.

48. The compound of claim 47, wherein R^{E} is halogen.

49. The compound of any one of claims 38-48, wherein m is 0.

50. The compound of any one of claims 1-49, wherein L is of Formula I-3: 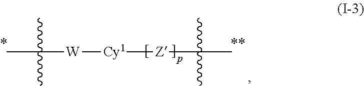
wherein:
* denotes attachment to T and ** denotes attachment to C;
W is absent; or
W is C₁₋₃ alkylene, —O—, —NR^{W}—, or —(C═O)—, wherein the alkylene is optionally substituted by one or more R^{u};
—Cy¹ is absent; or
—Cy¹ is 6-membered heteroarylene, C₆ arylene, C₃₋₁₂ carbocyclylene, or 3- to 12-membered heterocyclylene, wherein the arylene, heteroarylene, carbocyclylene, or heterocyclylene is optionally substituted by one or more R^{u};
Z′ is absent; or
each Z′ is independently C₁₋₃ alkylene, —O—, —NR^{W}—, —(C═O)—, C₃₋₁₂ carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted with one or more R^{u}; and
p is an integer selected from 0 to 8.

51. The compound of claim 50, wherein Cy¹ is C₃₋₁₂ carbocyclylene or 3- to 12-membered heterocyclylene, wherein the carbocyclylene or heterocyclylene is optionally substituted by one or more R^{u}.

52. The compound of claim 50, wherein Cy¹ is 3- to 12-membered heterocyclylene, wherein the heterocyclylene is optionally substituted by one or more R^{u}.

53. The compound of any one of claims 50-52, wherein W is absent.

54. The compound of any one of claims 50-52, wherein Z′ is absent.

55. The compound of any one of claims 50-52, wherein-[Z′] p- is-C(═O)—, C₁₋₆ alkylene, *-O-(C₁₋₆ alkylene)-, *-(C₁₋₆ alkylene)-(C(═O))—O—, *-(C₁₋₆ alkylene)-O—, *—C(═O)-(C₁₋₆ alkylene)-, *-(C₁₋₆ alkylene)-C(═O)—, 3- to 12-membered heterocyclylene, *—C(═O)-(3- to 12-membered heterocyclylene)-, *-(3- to 12-membered heterocyclylene)-C(═O)—, *-(3- to 12-membered heterocyclylene)-(C₁₋₆ alkylene)-, *-(C₁₋₆ alkylene)-(3- to 12-membered heterocyclylene)-, *-(C₁₋₆ alkylene)-(3- to 12-membered heterocyclylene)-(C₁₋₆ alkylene)-, *-(C₁₋₆ alkylene)-(3- to 12-membered heterocyclylene)-(C(═O))—, *-(C(═O))-(3- to 12-membered heterocyclylene)-(C₁₋₆ alkylene)-, *-(3- to 12-membered heterocyclylene)-(C₁₋₆ alkylene)-(C(═O))—, *-(C(═O))-(C₁₋₆ alkylene)-(3- to 12-membered heterocyclylene)-, *-(C₁₋₆ alkylene)-(C(═O))-(3- to 12-membered heterocyclylene)-, or *-(3- to 12-membered heterocyclylene)-(C(═O))-(C₁₋₆ alkylene)-, wherein the alkylene or heterocyclylene is optionally substituted by one or more R^{u}, and *denotes attachment to C.

56. The compound of claim 55, wherein-[Z′] p- is-C(═O)—, C₁₋₆ alkylene, *-(C₁₋₆ alkylene)-(C(═O))—O—, *—C(═O)-(C₁₋₆ alkylene)-, *-(C₁₋₆ alkylene)-C(═O)—, 3- to 12-membered heterocyclylene, *-(3- to 12-membered heterocyclylene)-(C₁₋₆ alkylene)-, *-(C(═O))-(3- to 12-membered heterocyclylene)-(C₁₋₆ alkylene)-, *-(C(═O))-(C₁₋₆ alkylene)-(3- to 12-membered heterocyclylene)-, *-(C₁₋₆ alkylene)-(C(═O))-(3- to 12-membered heterocyclylene)-, wherein the alkylene or heterocyclylene is optionally substituted by one or more R^{u}, and *denotes attachment to C.

57. A compound selected from the compounds in Tables 1 and 2, or a pharmaceutically acceptable salt thereof.

58. A compound selected from Table X, or a pharmaceutically acceptable salt thereof,
TABLE X

| Compound | | |
|---|---|---|
| No. | Structure | Chemical Name |
| A99 | 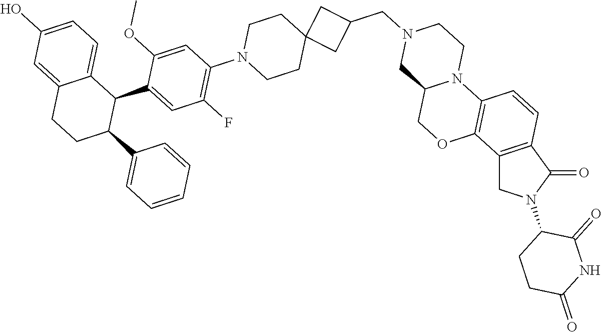 [SMILES: COc1cc(N2CCC3(CC2)CC(CN2CCN4c5ccc6c(c5OC[C@H]4C2)CN([C@H]2CCC(=O)NC2=O)C6=O)C3)c(F)cc1[C@@H]1c2ccc(O)cc2CC[C@@H]1c1ccccc1] | (S)-3-((R)-7-((7-(2-fluoro-4- ((1S,2S)-6-hydroxy-2-phenyl- 1,2,3,4-tetrahydronaphthalen-1- yl)-5-mcthoxyphenyl)-7- azaspiro[3.5]nonan-2- yl)methyl)-1-oxo- 1,3,5,5a,6,7,8,9-octahydro-2H- pyrazino[11,2′:4,5][1,4]oxazino [2,3-e]isoindol-2-yl)piperidine- 2,6-dione |
| A100 | 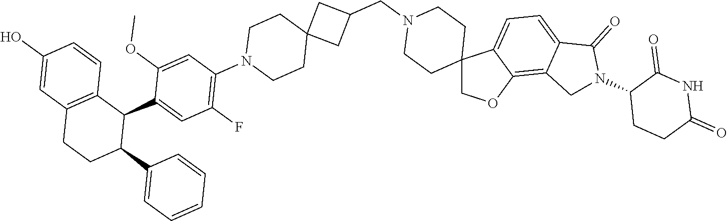 [SMILES: COc1cc(N2CCC3(CC2)CC(CN2CCC4(CC2)COc2c4ccc4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen-1- yl)-5-methoxyphenyl)-7- azaspiro[3.5]nonan-2- yl)methyl)-6-oxo-6,8-dihydro- 2H,7H-spiro[furo[2,3-e] isoindole-3,4′-piperidin]-7- yl)piperidine-2,6-dione |
| A129 | 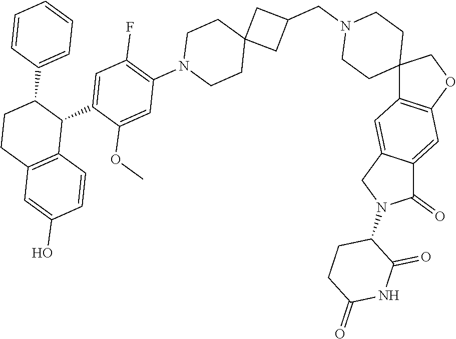 [SMILES: COc1cc(N2CCC3(CC2)CC(CN2CCC4(CC2)COc2cc5c(cc24)CN([C@H]2CCC(=O)NC2=O)C5=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen-1- yl)-5-methoxyphenyl)-7- azaspiro[3.5]nonan-2- yl)methyl)-7-oxo-5,7-dihydro- 2H,6H-spiro[furo[2,3- f]isoindole-3,4′-piperidin]-6- yl)piperidine-2,6-dione |
| A171 | 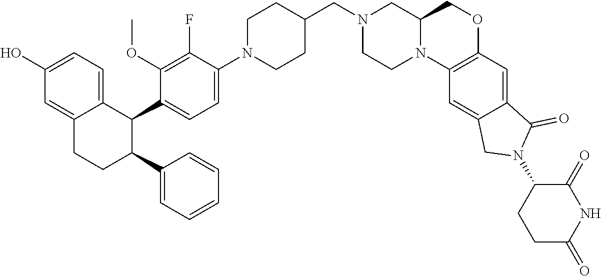 [SMILES: COc1c([C@@H]2c3ccc(O)cc3CC[C@@H]2c2ccccc2)ccc(N2CCC(CN3CCN4c5cc6c(cc5OC[C@@H]4C3)C(=O)N([C@H]3CCC(=O)NC3=O)C6)CC2)c1F] | (S)-3-((S)-3-((1-(2-fluoro-4- ((1S,2S)-6-hydroxy-2-phenyl- 1,2,3,4-tetrahydronaphthalen-1- yl)-3-methoxyphenyl)piperidin- 4-yl)methyl)-8-oxo- 1,2,3,4,4a,5,8,10-octahydro-9H- pyrazino[1′,2′:4,5][1,4]oxazino [2,3-f]isoindol-9-yl)piperidine- 2,6-dione |
| B366 | 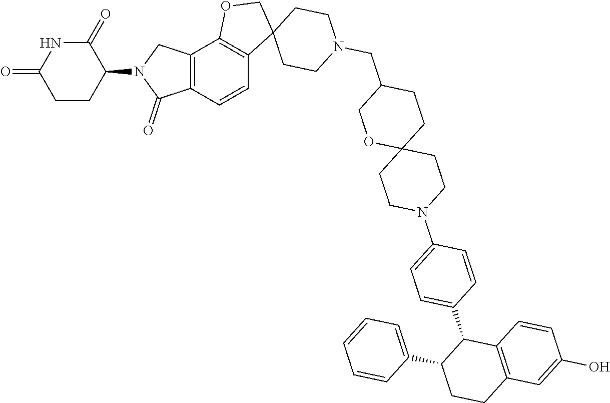 [SMILES: O=C1CC[C@H](N2Cc3c(ccc4c3OCC43CCN(CC4CCC5(CCN(c6ccc([C@@H]7c8ccc(O)cc8CC[C@@H]7c7ccccc7)cc6)CC5)OC4)CC3)C2=O)C(=O)N1] | (3S)-3-(1′-((9-(4-((1R,2S)-6- hydroxy-2-phenyl-1,2,3,4- tetrahydronaphthalen-1- yl)phenyl)-1-oxa-9- azaspiro[5.5]undecan-3- yl)methyl)-6-oxo-6,8-dihydro- 2H,7H-spiro[furo[2,3- e]isoindole-3,4′-piperidin]-7- yl)piperidine-2,6-dione |
| B366X | 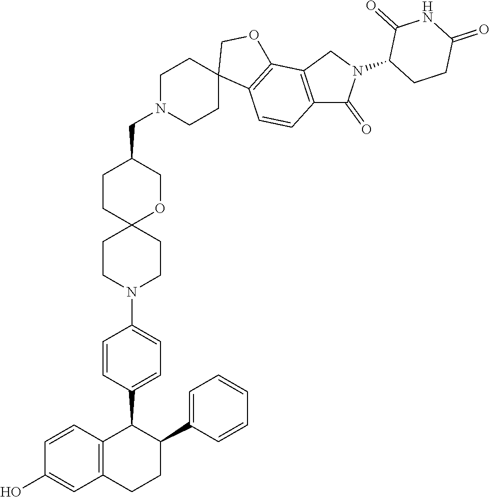 [SMILES: O=C1CC[C@H](N2Cc3c(ccc4c3OCC43CCN(C[C@@H]4CCC5(CCN(c6ccc([C@@H]7c8ccc(O)cc8CC[C@@H]7c7ccccc7)cc6)CC5)OC4)CC3)C2=O)C(=O)N1] | (S)-3-(1′-(((S)-9-(4-((1R,2S)-6- hydroxy-2-phenyl-1,2,3,4- tetrahydronaphthalen-1- yl)phenyl)-1-oxa-9- azaspiro[5.5]undecan-3- yl)methyl)-6-oxo-6,8-dihydro- 2H,7H-spiro[furo[2,3-e] isoindole-3,4′-piperidin]-7- yl)piperidine-2,6-dione |
| B366Y | 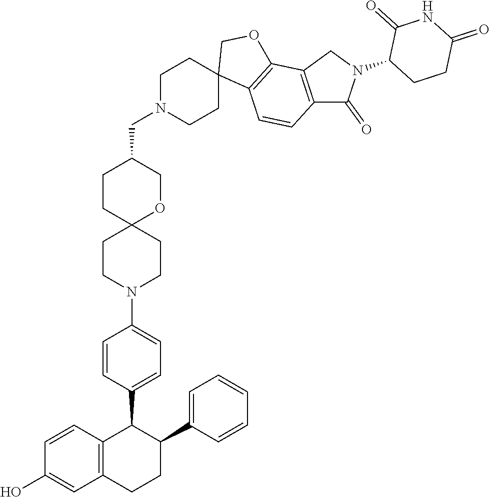 [SMILES: O=C1CC[C@H](N2Cc3c(ccc4c3OCC43CCN(C[C@H]4CCC5(CCN(c6ccc([C@@H]7c8ccc(O)cc8CC[C@@H]7c7ccccc7)cc6)CC5)OC4)CC3)C2=O)C(=O)N1] | (S)-3-(1′-(((R)-9-(4-((1R,2S)-6- hydroxy-2-phenyl-1,2,3,4- tetrahydronaphthalen-1- yl)phenyl)-1-oxa-9- azaspiro[5.5]undecan-3- yl)methyl)-6-oxo-6,8-dihydro- 2H,7H-spiro[furo[2,3- e]isoindole-3,4′-piperidin]-7- yl)piperidine-2,6-dione |
| B337X | 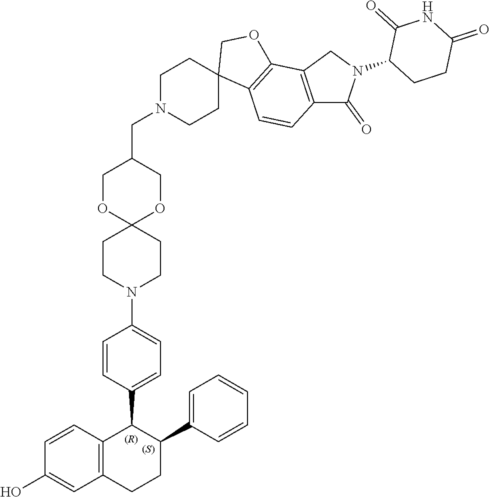 | (S)-3-(1′-((9-(4-((1R,2S)-6- hydroxy-2-phenyl-1,2,3,4- tetrahydronaphthalen-1- yl)phenyl)-1,5-dioxa-9- azaspiro[5.5]undecan-3- yl)methyl)-6-oxo-6,8-dihydro- 2H,7H-spiro[furo[2,3- e]isoindole-3,4′-piperidin]-7- yl)piperidine-2,6-dione |
| B306X | 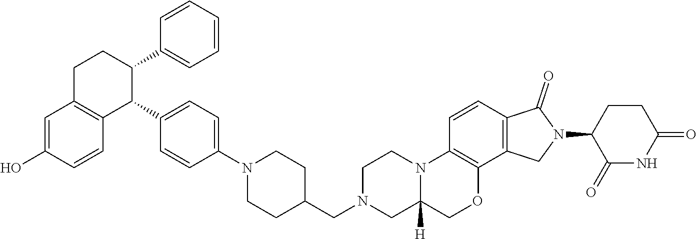 [SMILES: O=C1CC[C@H](N2Cc3c(ccc4c3OC[C@@H]3CN(CC5CCN(c6ccc([C@@H]7c8ccc(O)cc8CC[C@@H]7c7ccccc7)cc6)CC5)CCN43)C2=O)C(=O)N1] | (S)-3-((S)-7-((1-(4-((1R,2S)-6- hydroxy-2-phenyl-1,2,3,4- tetrahydronaphthalen-1- yl)phenyl)piperidin-4- yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| B308X | 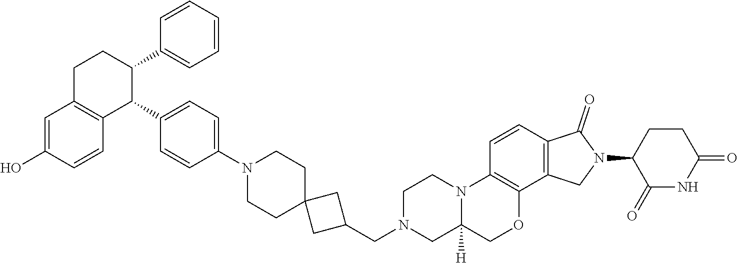 [SMILES: O=C1CC[C@H](N2Cc3c(ccc4c3OC[C@H]3CN(CC5CC6(CCN(c7ccc([C@@H]8c9ccc(O)cc9CC[C@@H]8c8ccccc8)cc7)CC6)C5)CCN43)C2=O)C(=O)N1] | (S)-3-((R)-7-((7-(4-((1R,2S)-6- hydroxy-2-phenyl-1,2,3,4- tetrahydronaphthalen-1- yl)phenyl)-7- azaspiro[3.5]nonan-2- yl)methyl)-1-oxo- 1,3,5,5a,6,7,8,9-octahydro-2H- pyrazino[11,2′:4,5][1,4]oxazino [2,3-e]isoindol-2-yl)piperidine- 2,6-dione |
| B330X | 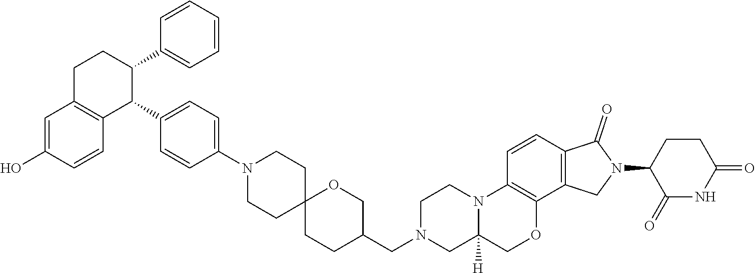 [SMILES: O=C1CC[C@H](N2Cc3c(ccc4c3OC[C@H]3CN(CC5CCC6(CCN(c7ccc([C@@H]8c9ccc(O)cc9CC[C@@H]8c8ccccc8)cc7)CC6)OC5)CCN43)C2=O)C(=O)N1] | (3S)-3-((5aR)-7-((9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A35 | 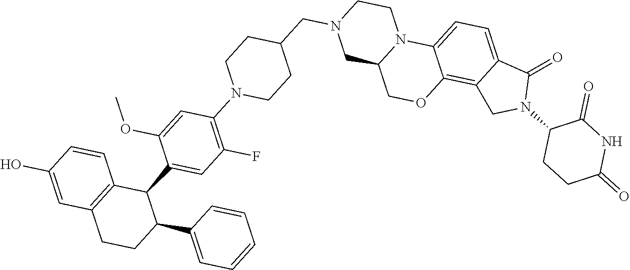 [SMILES: COc1cc(N2CCC(CN3CCN4c5ccc6c(c5OC[C@H]4C3)CN([C@H]3CCC(=O)NC3=O)C6=O)CC2)c(F)cc1[C@@H]1c2ccc(O)cc2CC[C@@H]1c1ccccc1] | (S)-3-((R)-7-((1-(2-fluoro-4- ((1S,2S)-6-hydroxy-2-phenyl- 1,2,3,4-tetrahydronaphthalen-1- yl)-5-methoxyphenyl)piperidin- 4-yl)methyl)-1-oxo- 1,3,5,5a,6,7,8,9-octahydro-2H- pyrazino[11,2′:4,5][1,4]oxazino [2,3-e]isoindol-2-yl)piperidine- 2,6-dione |
| B135X | 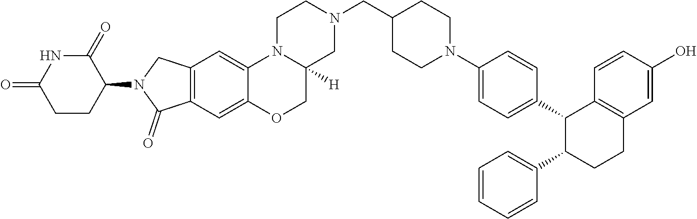 [SMILES: O=C1CC[C@H](N2Cc3cc4c(cc3C2=O)OC[C@@H]2CN(CC3CCN(c5ccc([C@@H]6c7ccc(O)cc7CC[C@@H]6c6ccccc6)cc5)CC3)CCN42)C(=O)N1] | (S)-3-((S)-3-((1-(4-((1R,2S)-6- hydroxy-2-phenyl-1,2,3,4- tetrahydronaphthalen-1- yl)phenyl)piperidin-4- yl)methyl)-8-oxo- 1,2,3,4,4a,5,8,10-octahydro-9H- pyrazino[11,2′:4,5][1,4]oxazino [2,3-f]isoindol-9-yl)piperidine- 2,6-dione |
| B211 | 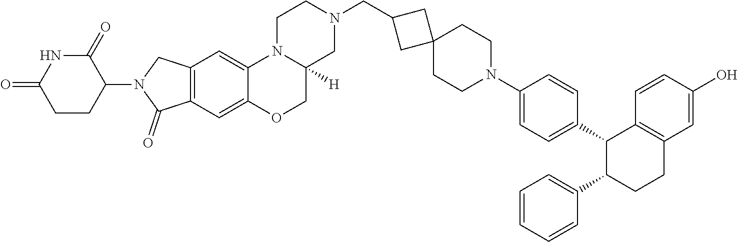 [SMILES: O=C1CCC(N2Cc3cc4c(cc3C2=O)OC[C@@H]2CN(CC3CC5(CCN(c6ccc([C@@H]7c8ccc(O)cc8CC[C@@H]7c7ccccc7)cc6)CC5)C3)CCN42)C(=O)N1] | 3-((S)-3-((7-(4-((1R,2S)-6- hydroxy-2-phenyl-1,2,3,4- tetrahydronaphthalen-1- yl)phenyl)-7- azaspiro[3.5]nonan-2- yl)methyl)-8-oxo- 1,2,3,4,4a,5,8,10-octahydro-9H- pyrazino[11,2′:4,5][1,4]oxazino [2,3-f]isoindol-9-yl)piperidine- 2,6-dione |
| B226X | 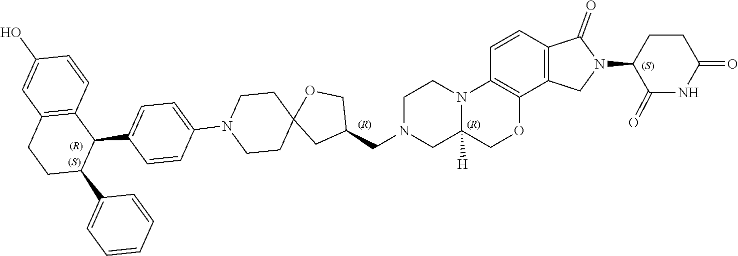 | (S)-3-((R)-7-(((R)-8-(4- ((1R,2S)-6-hydroxy-2-phenyl- 1,2,3,4-tetrahydronaphthalen-1- yl)phenyl)-1-oxa-8- azaspiro[4.5]decan-3-yl)methyl)- 1-oxo-1,3,5,5a,6,7,8,9- octahydro-2H- pyrazino[1′,2′:4,5][1,4]oxazino [2,3-e]isoindol-2-yl)piperidine- 2,6-dione |
| B226Y | 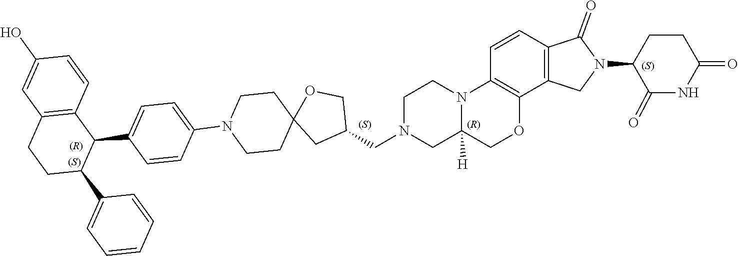 | (S)-3-((R)-7-(((S)-8-(4-((1R,2S)- 6-hydroxy-2-phenyl-1,2,3,4- tetrahydronaphthalen-1- yl)phenyl)-1-oxa-8- azaspiro[4.5]decan-3-yl)methyl)- 1-oxo-1,3,5,5a,6,7,8,9- octahydro-2H- pyrazino[11,2′:4,5][1,4]oxazino [2,3-e]isoindol-2-yl)piperidine- 2,6-dione |
| B227X | 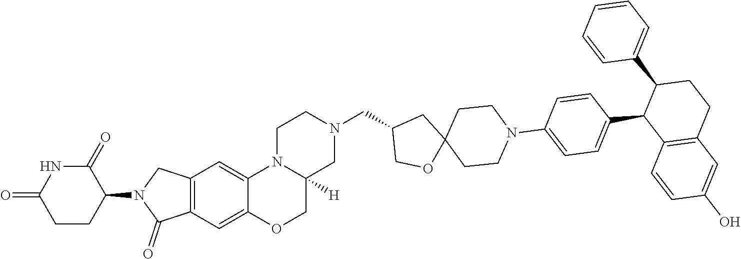 [SMILES: O=C1CC[C@H](N2Cc3cc4c(cc3C2=O)OC[C@@H]2CN(C[C@H]3COC5(CCN(c6ccc([C@@H]7c8ccc(O)cc8CC[C@@H]7c7ccccc7)cc6)CC5)C3)CCN42)C(=O)N1] | (S)-3-((S)-3-(((S)-8-(4-((1R,2S)- 6-hydroxy-2-phenyl-1,2,3,4- tetrahydronaphthalen-1- yl)phenyl)-1-oxa-8- azaspiro[4.5]decan-3-yl)methyl)- 8-oxo-1,2,3,4,4a,5,8,10- octahydro-9H- pyrazino[1′,2′:4,5][1,4]oxazino [2,3-f]isoindol-9-yl)piperidine- 2,6-dione |
| B227Y | 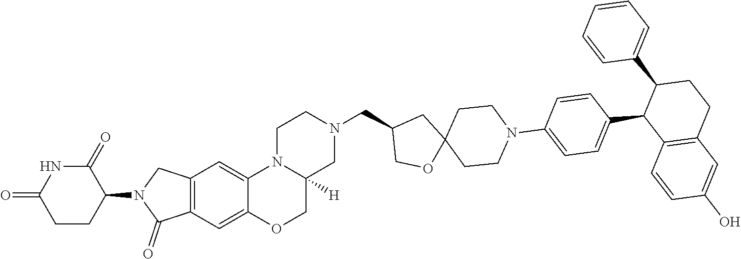 [SMILES: O=C1CC[C@H](N2Cc3cc4c(cc3C2=O)OC[C@@H]2CN(C[C@@H]3COC5(CCN(c6ccc([C@@H]7c8ccc(O)cc8CC[C@@H]7c7ccccc7)cc6)CC5)C3)CCN42)C(=O)N1] | (S)-3-((S)-3-(((R)-8-(4-((1R,2S)- 6-hydroxy-2-phenyl-1,2,3,4- tetrahydronaphthalen-1- yl)phenyl)-1-oxa-8- azaspiro[4.5]decan-3-yl)methyl)- 8-oxo-1,2,3,4,4a,5,8,10- octahydro-9H- pyrazino[11,2′:4,5][1,4]oxazino [2,3-f]isoindol-9-yl)piperidine- 2,6-dione |
| B229X | 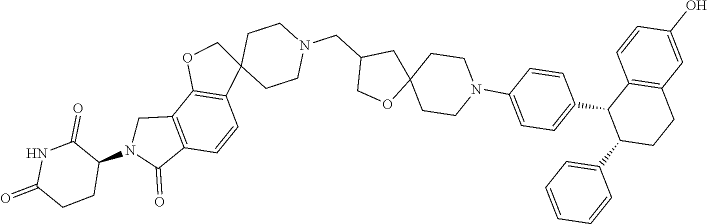 [SMILES: O=C1CC[C@H](N2Cc3c(ccc4c3OCC43CCN(CC4COC5(CCN(c6ccc([C@@H]7c8ccc(O)cc8CC[C@@H]7c7ccccc7)cc6)CC5)C4)CC3)C2=O)C(=O)N1] | (3S)-3-(1′-((8-(4-((1R,2S)-6- hydroxy-2-phenyl-1,2,3,4- tetrahydronaphthalen-1- yl)phenyl)-1-oxa-8- azaspiro[4.5]decan-3-yl)methyl)- 6-oxo-6,8-dihydro-2H,7H- spiro[furo[2,3-e]isoindole-3,4′- piperidin]-7-yl)piperidine-2,6- dione |
| A73 | 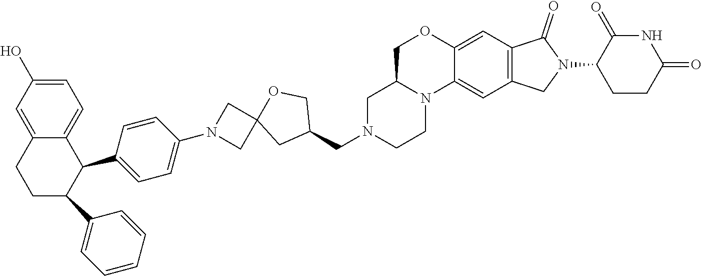 [SMILES: O=C1CC[C@H](N2Cc3cc4c(cc3C2=O)OC[C@@H]2CN(C[C@@H]3COC5(C3)CN(c3ccc([C@@H]6c7ccc(O)cc7CC[C@@H]6c6ccccc6)cc3)C5)CCN42)C(=O)N1] | (S)-3-((S)-3-(((R)-2-(4-((1R,2S)- 6-hydroxy-2-phenyl-1,2,3,4- tetrahydronaphthalen-1- yl)phenyl)-5-oxa-2- azaspiro[3.4]octan-7-yl)methyl)- 8-oxo-1,2,3,4,4a,5,8,10- octahydro-9H- pyrazino[11,2′:4,5][1,4]oxazino [2,3-f]isoindol-9-yl)piperidine- 2,6-dione |
| A40 | 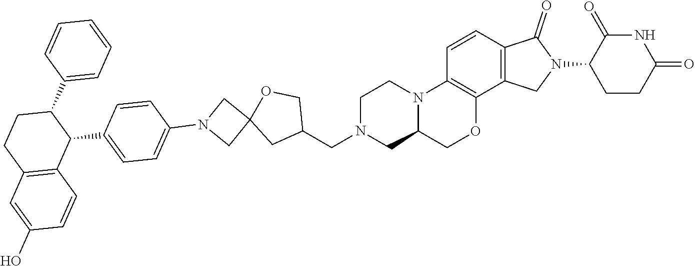 [SMILES: O=C1CC[C@H](N2Cc3c(ccc4c3OC[C@H]3CN(CC5COC6(C5)CN(c5ccc([C@@H]7c8ccc(O)cc8CC[C@@H]7c7ccccc7)cc5)C6)CCN43)C2=O)C(=O)N1] | (3S)-3-((5aR)-7-((2-(4-((1R,2S)- 6-hydroxy-2-phenyl-1,2,3,4- tetrahydronaphthalen-1- yl)phenyl)-5-oxa-2- azaspiro[3.4]octan-7-yl)methyl)- 1-oxo-1,3,5,5a,6,7,8,9- octahydro-2H- pyrazino[11,2′:4,5][1,4]oxazino [2,3-e]isoindol-2-yl)piperidine- 2,6-dione |
| A69 | 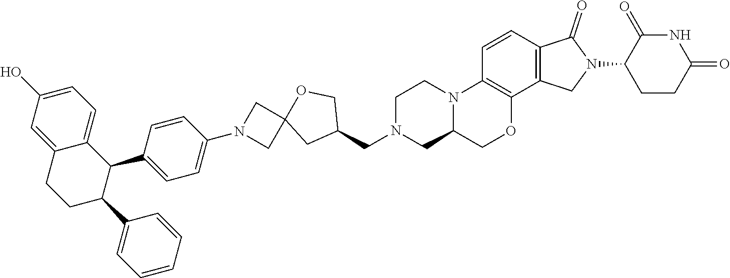 [SMILES: O=C1CC[C@H](N2Cc3c(ccc4c3OC[C@H]3CN(C[C@@H]5COC6(C5)CN(c5ccc([C@@H]7c8ccc(O)cc8CC[C@@H]7c7ccccc7)cc5)C6)CCN43)C2=O)C(=O)N1] | (S)-3-((R)-7-(((R)-2-(4- ((1R,2S)-6-hydroxy-2-phenyl- 1,2,3,4-tetrahydronaphthalen-1- yl)phenyl)-5-oxa-2- azaspiro[3.4]octan-7-yl)methyl)- 1-oxo-1,3,5,5a,6,7,8,9- octahydro-2H- pyrazino[11,2′:4,5][1,4]oxazino [2,3-e]isoindol-2-yl)piperidine- 2,6-dione |
| A76 | 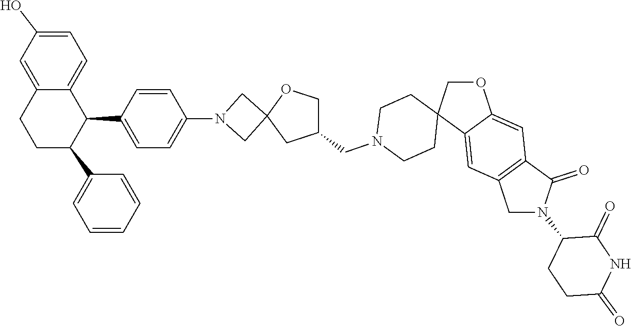 [SMILES: O=C1CC[C@H](N2Cc3cc4c(cc3C2=O)OCC42CCN(C[C@H]3COC4(C3)CN(c3ccc([C@@H]5c6ccc(O)cc6CC[C@@H]5c5ccccc5)cc3)C4)CC2)C(=O)N1] | (S)-3-(1′-(((S)-2-(4-((1R,2S)-6- hydroxy-2-phenyl-1,2,3,4- tetrahydronaphthalen-1- yl)phenyl)-5-oxa-2- azaspiro[3.4]octan-7-yl)methyl)- 7-oxo-5,7-dihydro-2H,6H- spiro[furo[2,3-f]isoindole-3,4′- piperidin]-6-yl)piperidine-2,6- dione |
| A77 | 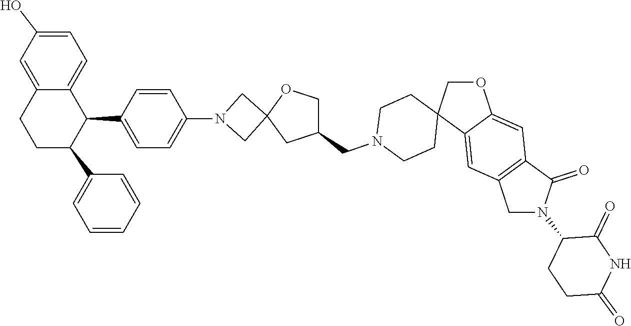 [SMILES: O=C1CC[C@H](N2Cc3cc4c(cc3C2=O)OCC42CCN(C[C@@H]3COC4(C3)CN(c3ccc([C@@H]5c6ccc(O)cc6CC[C@@H]5c5ccccc5)cc3)C4)CC2)C(=O)N1] | (S)-3-(1′-(((R)-2-(4-((1R,2S)-6- hydroxy-2-phenyl-1,2,3,4- tetrahydronaphthalen-1- yl)phenyl)-5-oxa-2- azaspiro[3.4]octan-7-yl)methyl)- 7-oxo-5,7-dihydro-2H,6H- spiro[furo[2,3-f]isoindole-3,4′- piperidin]-6-yl)piperidine-2,6- dione |
| A94 | 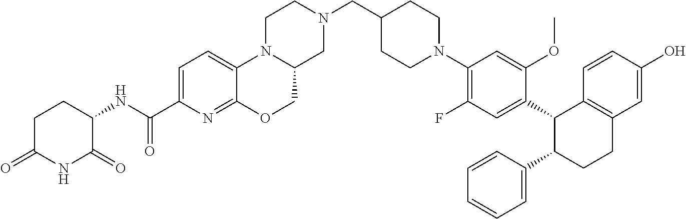 [SMILES: COc1cc(N2CCC(CN3CCN4c5ccc(C(=O)N[C@H]6CCC(=O)NC6=O)nc5OC[C@H]4C3)CC2)c(F)cc1[C@@H]1c2ccc(O)cc2CC[C@@H]1c1ccccc1] | (R)-N-((S)-2,6-dioxopiperidin-3- yl)-3-((1-(2-fluoro-4-((1S,2S)-6- hydroxy-2-phenyl-1,2,3,4- tetrahydronaphthalen-1-y1)-5- methoxyphenyl)piperidin-4- yl)methyl)-1,2,3,4,4a,5- hexahydropyrazino[1,2- d]pyrido[2,3-b][1,4]oxazine-8- carboxamide |
| A112 | 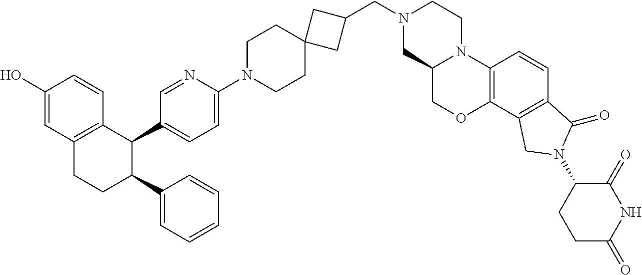 [SMILES: O=C1CC[C@H](N2Cc3c(ccc4c3OC[C@H]3CN(CC5CC6(CCN(c7ccc([C@@H]8c9ccc(O)cc9CC[C@@H]8c8ccccc8)cn7)CC6)C5)CCN43)C2=O)C(=O)N1] | (S)-3-((R)-7-((7-(5-((1S,2S)-6- hydroxy-2-phenyl-1,2,3,4- tetrahydronaphthalen-1- yl)pyridin-2-yl)-7- azaspiro[3.5]nonan-2- yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A121 | 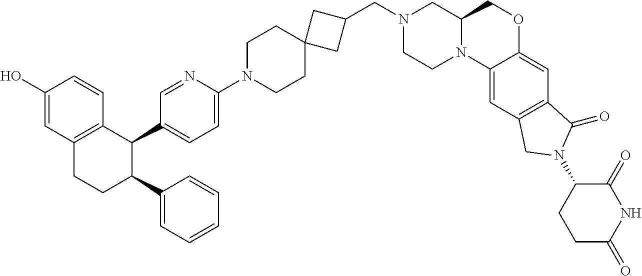 [SMILES: O=C1CC[C@H](N2Cc3cc4c(cc3C2=O)OC[C@@H]2CN(CC3CC5(CCN(c6ccc([C@@H]7c8ccc(O)cc8CC[C@@H]7c7ccccc7)cn6)CC5)C3)CCN42)C(=O)N1] | (S)-3-((S)-3-((7-(5-((1S,2S)-6- hydroxy-2-phenyl-1,2,3,4- tetrahydronaphthalen-1- yl)pyridin-2-yl)-7- azaspiro[3.5]nonan-2- yl)methyl)-8-oxo- 1,2,3,4,4a,5,8,10-octahydro-9H- pyrazino[11,2′:4,5][1,4]oxazino [2,3-f]isoindol-9-yl)piperidine- 2,6-dione |
| A130 | 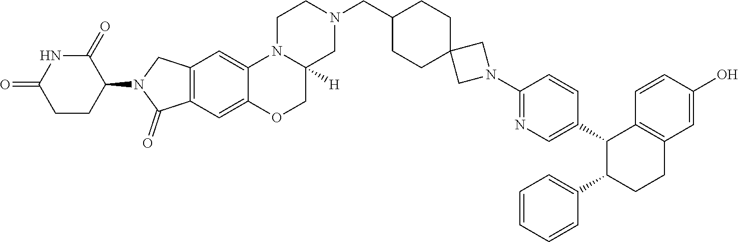 [SMILES: O=C1CC[C@H](N2Cc3cc4c(cc3C2=O)OC[C@@H]2CN(CC3CCC5(CC3)CN(c3ccc([C@@H]6c7ccc(O)cc7CC[C@@H]6c6ccccc6)cn3)C5)CCN42)C(=O)N1] | (S)-3-((S)-3-((2-(5-((1S,2S)-6- hydroxy-2-phenyl-1,2,3,4- tetrahydronaphthalen-1- yl)pyridin-2-yl)-2- azaspiro[3.5]nonan-7- yl)mcthyl)-8-oxo- 1,2,3,4,4a,5,8,10-octahydro-9H- pyrazino[11,2′:4,5][1,4]oxazino [2,3-f]isoindol-9-yl)piperidine- 2,6-dione |
| A131 | 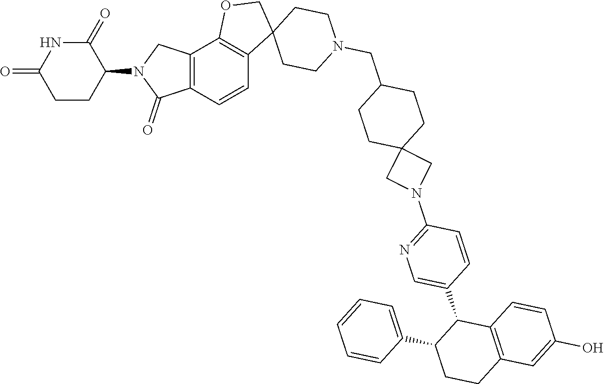 [SMILES: O=C1CC[C@H](N2Cc3c(ccc4c3OCC43CCN(CC4CCC5(CC4)CN(c4ccc([C@@H]6c7ccc(O)cc7CC[C@@H]6c6ccccc6)cn4)C5)CC3)C2=O)C(=O)N1] | (S)-3-(1′-((2-(5-((1S,2S)-6- hydroxy-2-phenyl-1,2,3,4- tetrahydronaphthalen-1- yl)pyridin-2-yl)-2- azaspiro[3.5]nonan-7- yl)methyl)-6-oxo-6,8-dihydro- 2H,7H-spiro[furo[2,3-e] isoindole-3,4′-piperidin]-7- yl)piperidine-2,6-dione |
| A133 | 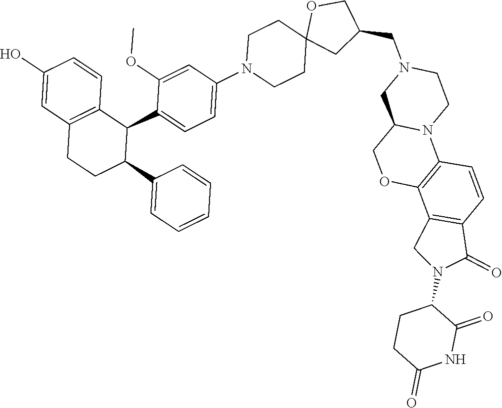 [SMILES: COc1cc(N2CCC3(CC2)C[C@H](CN2CCN4c5ccc6c(c5OC[C@H]4C2)CN([C@H]2CCC(=O)NC2=O)C6=O)CO3)ccc1[C@@H]1c2ccc(O)cc2CC[C@@H]1c1ccccc1] | (S)-3-((R)-7-(((R)-8-(4-((1S,2S)- 6-hydroxy-2-phenyl-1,2,3,4- tetrahydronaphthalen-1-yl)-3- methoxyphenyl)-1-oxa-8- azaspiro[4.5]decan-3-yl)methyl)- 1-oxo-1,3,5,5a,6,7,8,9- octahydro-2H- pyrazino[11,2′:4,5][1,4]oxazino [2,3-e]isoindol-2-yl)piperidine- 2,6-dione |
| A134 | 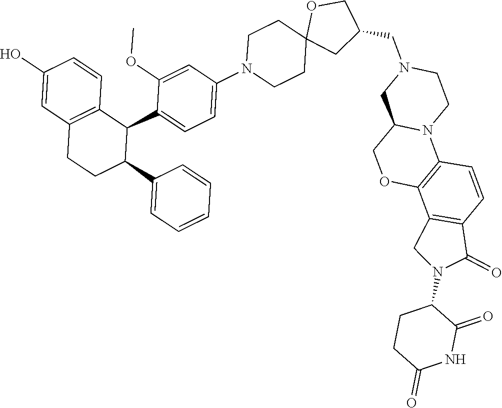 [SMILES: COc1cc(N2CCC3(CC2)C[C@@H](CN2CCN4c5ccc6c(c5OC[C@H]4C2)CN([C@H]2CCC(=O)NC2=O)C6=O)CO3)ccc1[C@@H]1c2ccc(O)cc2CC[C@@H]1c1ccccc1] | (S)-3-((R)-7-(((S)-8-(4-((1S,2S)- 6-hydroxy-2-phenyl-1,2,3,4- tetrahydronaphthalen-1-yl)-3- methoxyphenyl)-1-oxa-8- azaspiro[4.5]decan-3-yl)methyl)- 1-oxo-1,3,5,5a,6,7,8,9- octahydro-2H- pyrazino[1′,2′:4,5][1,4]oxazino [2,3-e]isoindol-2-yl)piperidine- 2,6-dione |
| A135 | 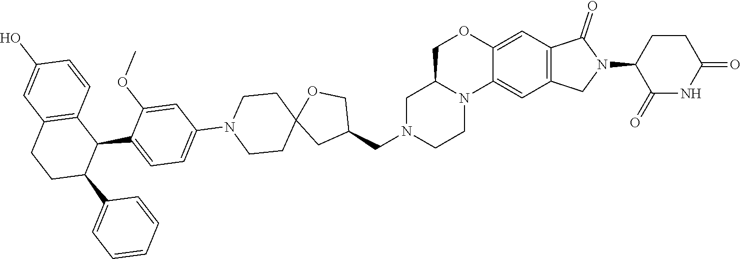 [SMILES: COc1cc(N2CCC3(CC2)C[C@H](CN2CCN4c5cc6c(cc5OC[C@@H]4C2)C(=O)N([C@H]2CCC(=O)NC2=O)C6)CO3)ccc1[C@@H]1c2ccc(O)cc2CC[C@@H]1c1ccccc1] | (S)-3-((S)-3-(((R)-8-(4-((1S,2S)- 6-hydroxy-2-phenyl-1,2,3,4- tetrahydronaphthalen-1-yl)-3- methoxyphenyl)-1-oxa-8- azaspiro[4.5]decan-3-yl)methyl)- 8-oxo-1,2,3,4,4a,5,8,10- octahydro-9H- pyrazino[1′,2′:4,5][1,4]oxazino [2,3-f]isoindol-9-yl)piperidine- 2,6-dione |
| A136 | 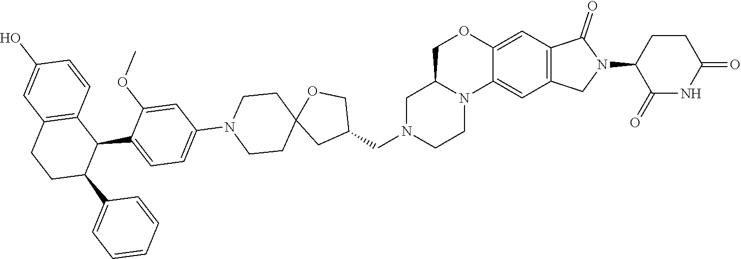 [SMILES: COc1cc(N2CCC3(CC2)C[C@@H](CN2CCN4c5cc6c(cc5OC[C@@H]4C2)C(=O)N([C@H]2CCC(=O)NC2=O)C6)CO3)ccc1[C@@H]1c2ccc(O)cc2CC[C@@H]1c1ccccc1] | (S)-3-((S)-3-(((S)-8-(4-((1S,2S)- 6-hydroxy-2-phenyl-1,2,3,4- tetrahydronaphthalen-1-yl)-3- methoxyphenyl)-1-oxa-8- azaspiro[4.5]decan-3-yl)methyl)- 8-oxo-1,2,3,4,4a,5,8,10- octahydro-9H- pyrazino[1′,2′:4,5][1,4]oxazino [2,3-f]isoindol-9-yl)piperidine- 2,6-dione |
| A140 | 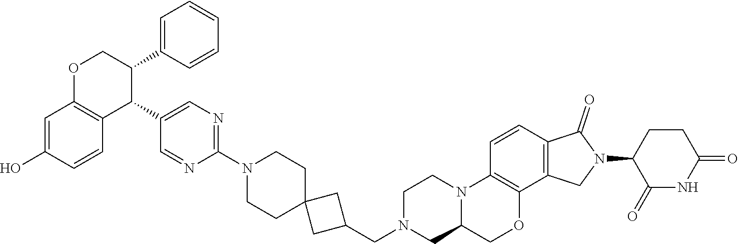 [SMILES: O=C1CC[C@H](N2Cc3c(ccc4c3OC[C@H]3CN(CC5CC6(CCN(c7ncc([C@@H]8c9ccc(O)cc9OC[C@@H]8c8ccccc8)cn7)CC6)C5)CCN43)C2=O)C(=O)N1] | (S)-3-((R)-7-((7-(5-((1S,2S)-6- hydroxy-2-phenyl-1,2,3,4- tetrahydronaphthalen-1- yl)pyrimidin-2-yl)-7- azaspiro[3.5]nonan-2- yl)methyl)-1-oxo- 1,3,5,5a,6,7,8,9-octahydro-2H- pyrazino[11,2′:4,5][1,4]oxazino [2,3-e]isoindol-2-yl)piperidine- 2,6-dione |
| A154 | 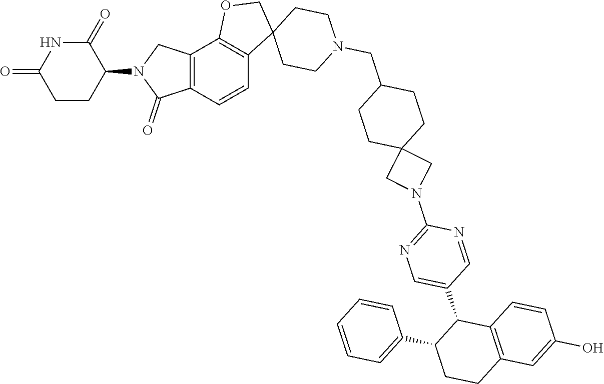 [SMILES: O=C1CC[C@H](N2Cc3c(ccc4c3OCC43CCN(CC4CCC5(CC4)CN(c4ncc([C@@H]6c7ccc(O)cc7CC[C@@H]6c6ccccc6)cn4)C5)CC3)C2=O)C(=O)N1] | (S)-3-(1′-((2-(5-((1S,2S)-6- hydroxy-2-phenyl-1,2,3,4- tetrahydronaphthalen-1- yl)pyrimidin-2-yl)-2- azaspiro[3.5]nonan-7- yl)methyl)-6-oxo-6,8-dihydro- 2H,7H-spiro[furo[2,3-e] isoindole-3,4′-piperidin]-7- yl)piperidine-2,6-dione |
| A163 | 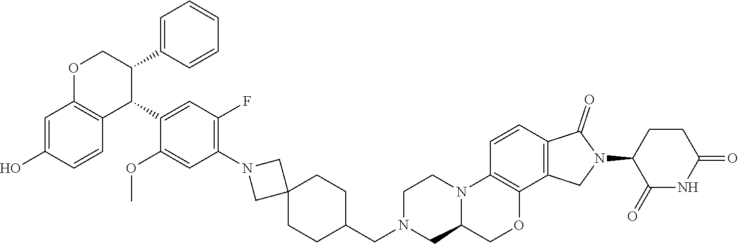 [SMILES: COc1cc(N2CC3(CCC(CN4CCN5c6ccc7c(c6OC[C@H]5C4)CN([C@H]4CCC(=O)NC4=O)C7=O)CC3)C2)c(F)cc1[C@@H]1c2ccc(O)cc2OC[C@@H]1c1ccccc1] | (S)-3-((R)-7-((2-(2-fluoro-4- ((1S,2S)-6-hydroxy-2-phenyl- 1,2,3,4-tetrahydronaphthalen-1- yl)-5-methoxyphenyl)-2- azaspiro[3.5]nonan-7- yl)methyl)-1-oxo- 1,3,5,5a,6,7,8,9-octahydro-2H- pyrazino[1′,2′:4,5][1,4]oxazino [2,3-e]isoindol-2-yl)piperidine- 2,6-dione |
| A164 | 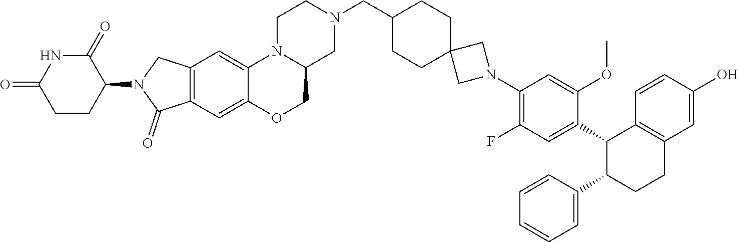 [SMILES: COc1cc(N2CC3(CCC(CN4CCN5c6cc7c(cc6OC[C@@H]5C4)C(=O)N([C@H]4CCC(=O)NC4=O)C7)CC3)C2)c(F)cc1[C@@H]1c2ccc(O)cc2CC[C@@H]1c1ccccc1] | (S)-3-((S)-3-((2-(2-fluoro-4- ((1S,2S)-6-hydroxy-2-phenyl- 1,2,3,4-tetrahydronaphthalen-1- yl)-5-methoxyphenyl)-2- azaspiro[3.5]nonan-7- yl)methyl)-8-oxo- 1,2,3,4,4a,5,8,10-octahydro-9H- pyrazino[11,2′:4,5][1,4]oxazino [2,3-f]isoindol-9-yl)piperidine- 2,6-dione |
| A91 | 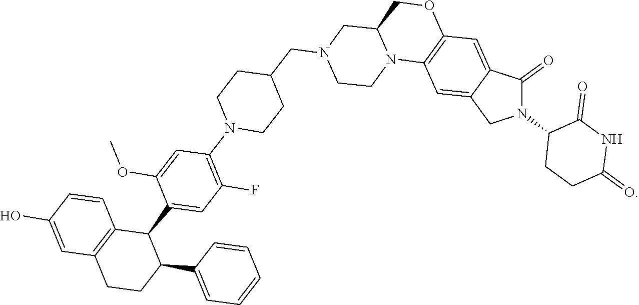 [SMILES: COc1cc(N2CCC(CN3CCN4c5cc6c(cc5OC[C@@H]4C3)C(=O)N([C@H]3CCC(=O)NC3=O)C6)CC2)c(F)cc1[C@@H]1c2ccc(O)cc2CC[C@@H]1c1ccccc1] | (S)-3-((S)-3-((1-(2-fluoro-4- ((1S,2S)-6-hydroxy-2-phenyl- 1,2,3,4-tetrahydronaphthalen-1- yl)-5-methoxyphenyl)piperidin- 4-yl)methyl)-8-oxo- 1,2,3,4,4a,5,8,10-octahydro-9H- pyrazino[11,2′:4,5][1,4]oxazino [2,3-f]isoindol-9-yl)piperidine- 2,6-dione |
| A173 | 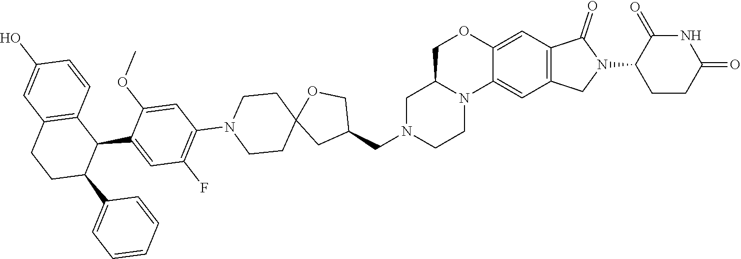 [SMILES: COc1cc(N2CCC3(CC2)C[C@H](CN2CCN4c5cc6c(cc5OC[C@@H]4C2)C(=O)N([C@H]2CCC(=O)NC2=O)C6)CO3)c(F)cc1[C@@H]1c2ccc(O)cc2CC[C@@H]1c1ccccc1] | (S)-3-((S)-3-(((R)-8-(2-fluoro-4- ((1S,2S)-6-hydroxy-2-phenyl- 1,2,3,4-tetrahydronaphthalen-1- yl)-5-methoxyphenyl)-1-oxa-8- azaspiro[4.5]decan-3-yl)methyl)- 8-oxo-1,2,3,4,4a,5,8,10- octahydro-9H- pyrazino[11,2′:4,5][1,4]oxazino [2,3-f]isoindol-9-yl)piperidine- 2,6-dione |
| A174 | 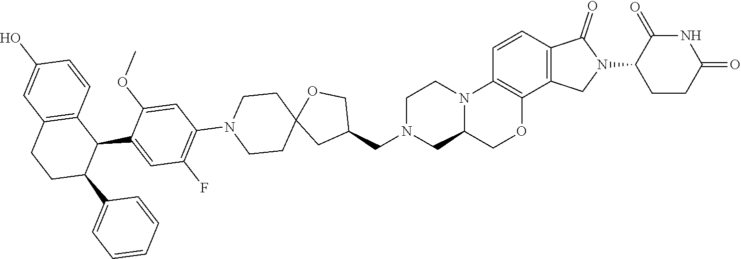 [SMILES: COc1cc(N2CCC3(CC2)C[C@H](CN2CCN4c5ccc6c(c5OC[C@H]4C2)CN([C@H]2CCC(=O)NC2=O)C6=O)CO3)c(F)cc1[C@@H]1c2ccc(O)cc2CC[C@@H]1c1ccccc1] | (S)-3-((R)-7-(((R)-8-(2-fluoro-4- ((1S,2S)-6-hydroxy-2-phenyl- 1,2,3,4-tetrahydronaphthalen-1- yl)-5-methoxyphenyl)-1-oxa-8- azaspiro[4.5]decan-3-yl)methyl)- 1-oxo-1,3,5,5a,6,7,8,9- octahydro-2H- pyrazino[1′,2′:4,5][1,4]oxazino [2,3-e]isoindol-2-yl)piperidine- 2,6-dione |
| A175 | 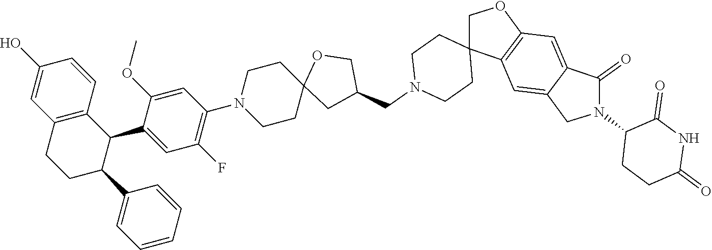 [SMILES: COc1cc(N2CCC3(CC2)C[C@H](CN2CCC4(CC2)COc2cc5c(cc24)CN([C@H]2CCC(=O)NC2=O)C5=O)CO3)c(F)cc1[C@@H]1c2ccc(O)cc2CC[C@@H]1c1ccccc1] | (S)-3-(1′-(((R)-8-(2-fluoro-4- ((1S,2S)-6-hydroxy-2-phenyl- 1,2,3,4-tetrahydronaphthalen-1- yl)-5-methoxyphenyl)-1-oxa-8- azaspiro[4.5]decan-3-yl)methyl)- 7-oxo-5,7-dihydro-2H,6H- spiro[furo[2,3-f]isoindole-3,4′- piperidin]-6-yl)piperidine-2,6- dione |
| A176 | 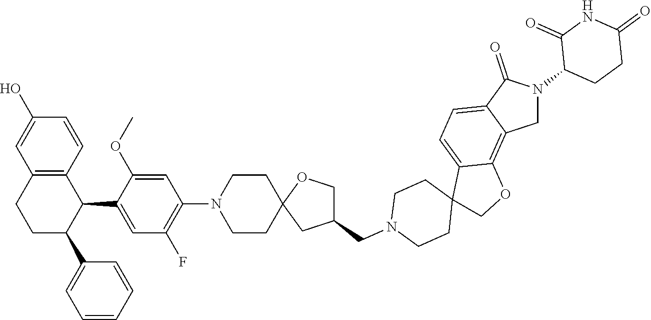 [SMILES: COc1cc(N2CCC3(CC2)C[C@H](CN2CCC4(CC2)COc2c4ccc4c2CN([C@H]2CCC(=O)NC2=O)C4=O)CO3)c(F)cc1[C@@H]1c2ccc(O)cc2CC[C@@H]1c1ccccc1] | (S)-3-(1′-(((R)-8-(2-fluoro-4- ((1S,2S)-6-hydroxy-2-phenyl- 1,2,3,4-tetrahydronaphthalen-1- yl)-5-methoxyphenyl)-1-oxa-8- azaspiro[4.5]decan-3-yl)methyl)- 6-oxo-6,8-dihydro-2H,7H- spiro[furo[2,3-e]isoindole-3,4′- piperidin]-7-yl)piperidinc-2,6- dione |
| A203 | 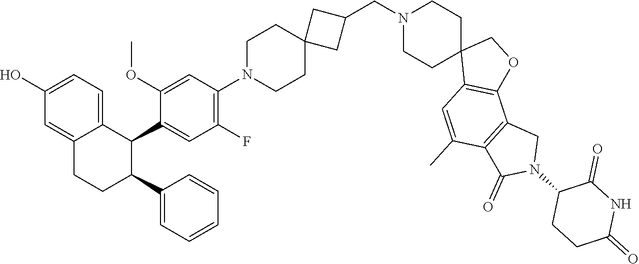 [SMILES: COc1cc(N2CCC3(CC2)CC(CN2CCC4(CC2)COc2c4cc(C)c4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen- 1-yl)-5-methoxyphenyl)-7- azaspiro[3.5]nonan-2- yl)methyl)-5-methyl-6-oxo- 6,8-dihydro-2H,7H- spiro[furo[2,3-e]isoindole- 3,4′-piperidin]-7- yl)piperidine-2,6-dione |
| A209 | 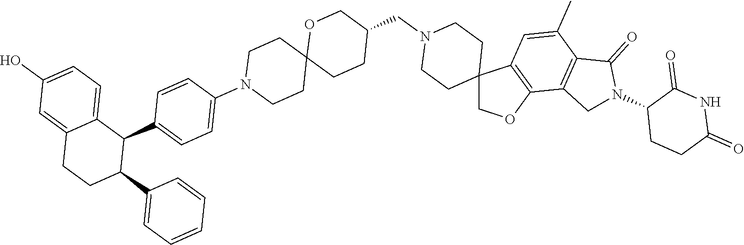 [SMILES: Cc1cc2c(c3c1C(=O)N([C@H]1CCC(=O)NC1=O)C3)OCC21CCN(C[C@@H]2CCC3(CCN(c4ccc([C@@H]5c6ccc(O)cc6CC[C@@H]5c5ccccc5)cc4)CC3)OC2)CC1] | (S)-3-(1′-(((S)-9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-5-methyl-6-oxo- 6,8-dihydro-2H,7H- spiro[furo[2,3-e]isoindole- 3,4′-piperidin]-7- yl)piperidine-2,6-dione |
| A210 | 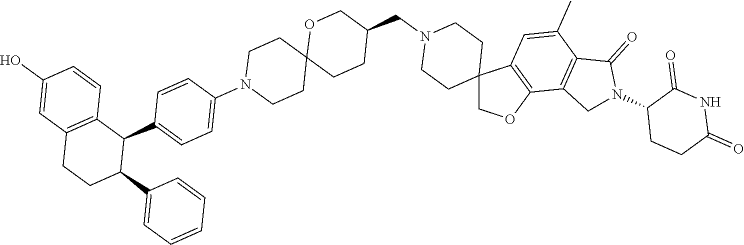 [SMILES: Cc1cc2c(c3c1C(=O)N([C@H]1CCC(=O)NC1=O)C3)OCC21CCN(C[C@H]2CCC3(CCN(c4ccc([C@@H]5c6ccc(O)cc6CC[C@@H]5c5ccccc5)cc4)CC3)OC2)CC1] | (S)-3-(1′-(((R)-9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-5-methyl-6-oxo- 6,8-dihydro-2H,7H- spiro[furo[2,3-e]isoindole- 3,4′-piperidin]-7- yl)piperidine-2,6-dione |
| A222 | 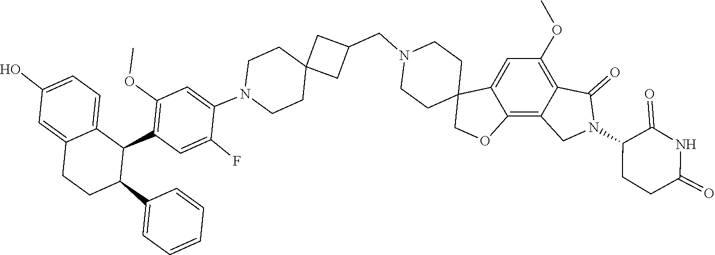 [SMILES: COc1cc(N2CCC3(CC2)CC(CN2CCC4(CC2)COc2c4cc(OC)c4c2CN([C@H]2CCC(=O)NC2=O)C4=O)C3)c(F)cc1[C@@H]1c2ccc(O)cc2CC[C@@H]1c1ccccc1] | (S)-3-(1′-((7-(2-fluoro-4- ((1S,2S)-6-hydroxy-2-phenyl- 1,2,3,4-tetrahydronaphthalen- 1-yl)-5-methoxyphenyl)-7- azaspiro[3.5]nonan-2- yl)methyl)-5-methoxy-6-oxo- 6,8-dihydro-2H,7H- spiro[furo[2,3-e]isoindole- 3,4′-piperidin]-7- yl)piperidine-2,6-dione |
| A223 | 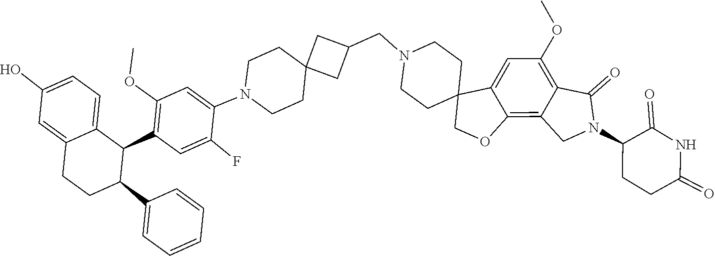 [SMILES: COc1cc(N2CCC3(CC2)CC(CN2CCC4(CC2)COc2c4cc(OC)c4c2CN([C@@H]2CCC(=O)NC2=O)C4=O)C3)c(F)cc1[C@@H]1c2ccc(O)cc2CC[C@@H]1c1ccccc1] | (R)-3-(1′-((7-(2-fluoro-4- ((1S,2S)-6-hydroxy-2-phenyl- 1,2,3,4-tetrahydronaphthalen- 1-yl)-5-methoxyphenyl)-7- azaspiro[3.5]nonan-2- yl)methyl)-5-methoxy-6-oxo- 6,8-dihydro-2H,7H- spiro[furo[2,3-e]isoindole- 3,4′-piperidin]-7- yl)piperidine-2,6-dione |
| A225 | 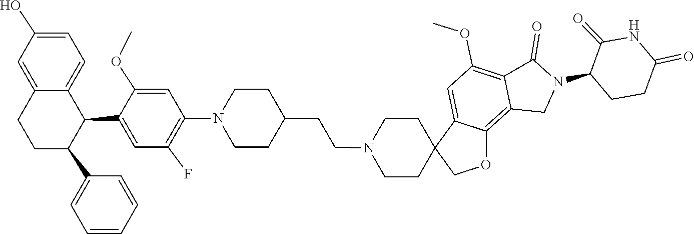 [SMILES: COc1cc(N2CCC(CCN3CCC4(CC3)COc3c4cc(OC)c4c3CN([C@@H]3CCC(=O)NC3=O)C4=O)CC2)c(F)cc1[C@@H]1c2ccc(O)cc2CC[C@@H]1c1ccccc1] | (R)-3-(1′-(2-(1-(2-fluoro-4- ((1S,2S)-6-hydroxy-2-phenyl- 1,2,3,4-tetrahydronaphthalen- 1-yl)-5- methoxyphenyl)piperidin-4- yl)ethyl)-5-methoxy-6-oxo- 6,8-dihydro-2H,7H- spiro[furo[2,3-e]isoindole- 3,4′-piperidin]-7- yl)piperidine-2,6-dione |
| A226 | 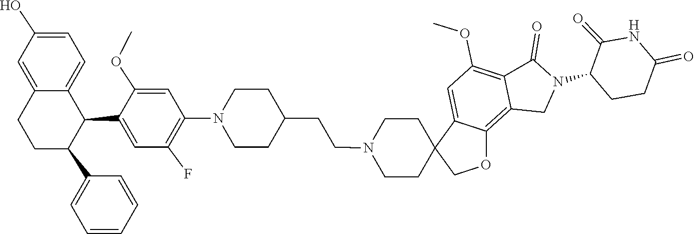 [SMILES: COc1cc(N2CCC(CCN3CCC4(CC3)COc3c4cc(OC)c4c3CN([C@H]3CCC(=O)NC3=O)C4=O)CC2)c(F)cc1[C@@H]1c2ccc(O)cc2CC[C@@H]1c1ccccc1] | (S)-3-(1′-(2-(1-(2-fluoro-4- ((1S,2S)-6-hydroxy-2-phenyl- 1,2,3,4-tetrahydronaphthalen- 1-yl)-5- methoxyphenyl)piperidin-4- yl)ethyl)-5-methoxy-6-oxo- 6,8-dihydro-2H,7H- spiro[furo[2,3-e]isoindole- 3,4′-piperidin]-7- yl)piperidine-2,6-dione |
| A227 | 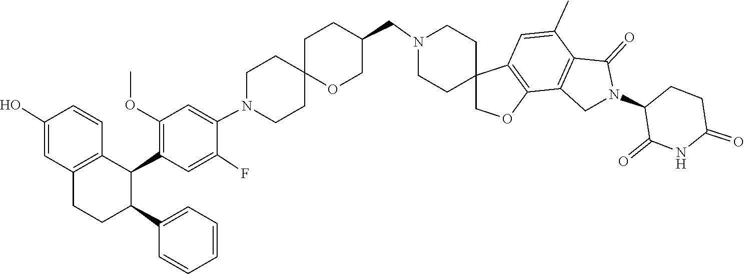 [SMILES: COc1cc(N2CCC3(CC[C@@H](CN4CCC5(CC4)COc4c5cc(C)c5c4CN([C@H]4CCC(=O)NC4=O)C5=O)CO3)CC2)c(F)cc1[C@@H]1c2ccc(O)cc2CC[C@@H]1c1ccccc1] | (S)-3-(1′-(((S)-9-(2-fluoro-4- ((1S,2S)-6-hydroxy-2-phenyl- 1,2,3,4-tetrahydronaphthalen- 1-yl)-5-methoxyphenyl)-1- oxa-9-azaspiro[5.5]undecan- 3-yl)methyl)-5-methyl-6-oxo- 6,8-dihydro-2H,7H- spiro[furo[2,3-e]isoindole- 3,4′-piperidin]-7- yl)piperidine-2,6-dione |
| A230 | 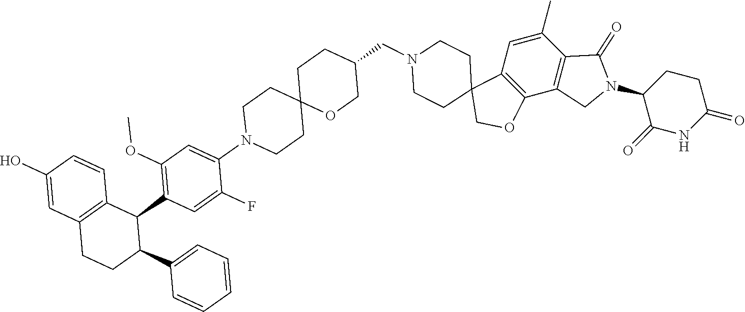 [SMILES: COc1cc(N2CCC3(CC[C@H](CN4CCC5(CC4)COc4c5cc(C)c5c4CN([C@H]4CCC(=O)NC4=O)C5=O)CO3)CC2)c(F)cc1[C@@H]1c2ccc(O)cc2CC[C@@H]1c1ccccc1] | (S)-3-(1′-(((R)-9-(2-fluoro-4- ((1S,2S)-6-hydroxy-2-phenyl- 1,2,3,4-tetrahydronaphthalen- 1-yl)-5-methoxyphenyl)-1- oxa-9-azaspiro[5.5]undecan- 3-yl)methyl)-5-methyl-6-oxo- 6,8-dihydro-2H,7H- spiro[furo[2,3-e]isoindole- 3,4′-piperidin]-7- yl)piperidine-2,6-dione |
| A231 | 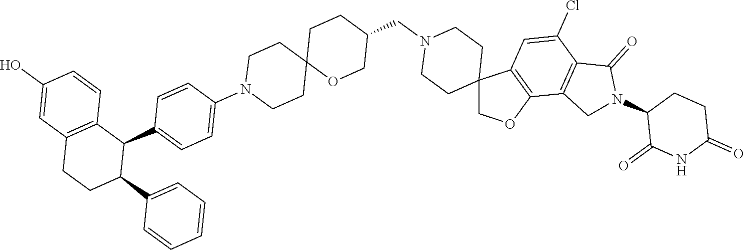 [SMILES: O=C1CC[C@H](N2Cc3c4c(cc(Cl)c3C2=O)C2(CCN(C[C@H]3CCC5(CCN(c6ccc([C@@H]7c8ccc(O)cc8CC[C@@H]7c7ccccc7)cc6)CC5)OC3)CC2)CO4)C(=O)N1] | (S)-3-(5-chloro-1′-(((R)-9-(4- ((1R,2S)-6-hydroxy-2-phenyl- 1,2,3,4-tetrahydronaphthalen- 1-yl)phenyl)-1-oxa-9- azaspiro[5.5]undecan-3- yl)methyl)-6-oxo-6,8- dihydro-2H,7H- spiro[furo[2,3-e]isoindole- 3,4′-piperidin]-7- yl)piperidine-2,6-dione |
| A232 | 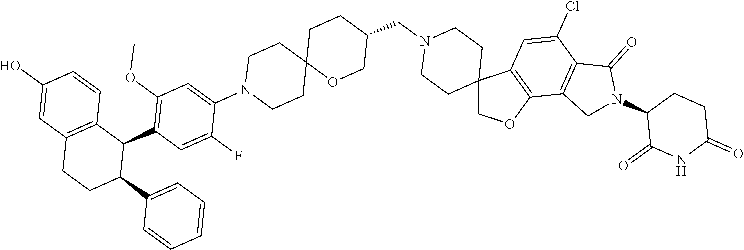 [SMILES: COc1cc(N2CCC3(CC[C@H](CN4CCC5(CC4)COc4c5cc(Cl)c5c4CN([C@H]4CCC(=O)NC4=O)C5=O)CO3)CC2)c(F)cc1[C@@H]1c2ccc(O)cc2CC[C@@H]1c1ccccc1] | (S)-3-(5-chloro-1′-(((R)-9-(2- fluoro-4-((1S,2S)-6-hydroxy- 2-phenyl-1,2,3,4- tetrahydronaphthalen-1-yl)-5- methoxyphenyl)-1-oxa-9- azaspiro[5.5]undecan-3- yl)methyl)-6-oxo-6,8- dihydro-2H,7H- spiro[furo[2,3-e]isoindole- 3,4′-piperidin]-7- yl)piperidine-2,6-dione |
| A233 | 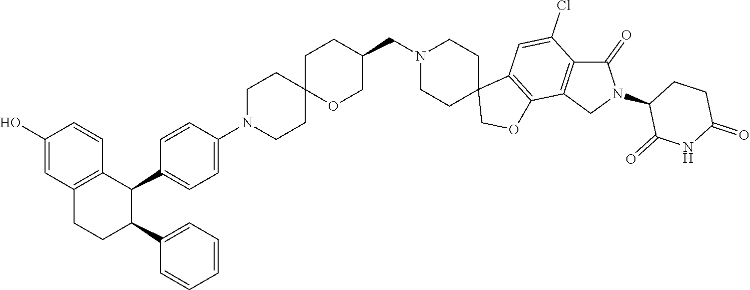 [SMILES: O=C1CC[C@H](N2Cc3c4c(cc(Cl)c3C2=O)C2(CCN(C[C@@H]3CCC5(CCN(c6ccc([C@@H]7c8ccc(O)cc8CC[C@@H]7c7ccccc7)cc6)CC5)OC3)CC2)CO4)C(=O)N1] | (S)-3-(5-chloro-1′-(((S)-9-(4- ((1R,2S)-6-hydroxy-2-phenyl- 1,2,3,4-tetrahydronaphthalen- 1-yl)phenyl)-1-oxa-9- azaspiro[5.5]undecan-3- yl)methyl)-6-oxo-6,8- dihydro-2H,7H- spiro[furo[2,3-e]isoindole- 3,4′-piperidin]-7- yl)piperidine-2,6-dione |
| A234 | 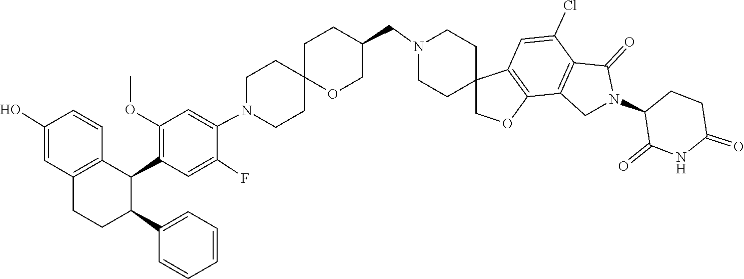 [SMILES: COc1cc(N2CCC3(CC[C@@H](CN4CCC5(CC4)COc4c5cc(Cl)c5c4CN([C@H]4CCC(=O)NC4=O)C5=O)CO3)CC2)c(F)cc1[C@@H]1c2ccc(O)cc2CC[C@@H]1c1ccccc1] | (S)-3-(5-chloro-1′-(((S)-9-(2- fluoro-4-((1S,2S)-6-hydroxy- 2-phenyl-1,2,3,4- tetrahydronaphthalen-1-yl)-5- methoxyphenyl)-1-oxa-9- azaspiro[5.5]undecan-3- yl)methyl)-6-oxo-6,8- dihydro-2H,7H- spiro[furo[2,3-e]isoindole- 3,4′-piperidin]-7- yl)piperidine-2,6-dione |
| A235 | 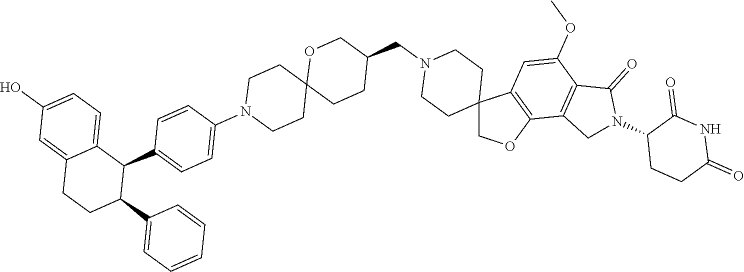 [SMILES: COc1cc2c(c3c1C(=O)N([C@H]1CCC(=O)NC1=O)C3)OCC21CCN(C[C@H]2CCC3(CCN(c4ccc([C@@H]5c6ccc(O)cc6CC[C@@H]5c5ccccc5)cc4)CC3)OC2)CC1] | (S)-3-(1′-(((R)-9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-5-methoxy-6-oxo- 6,8-dihydro-2H,7H- spiro[furo[2,3-e]isoindole- 3,4′-piperidin]-7- yl)piperidine-2,6-dione |
| A236 | 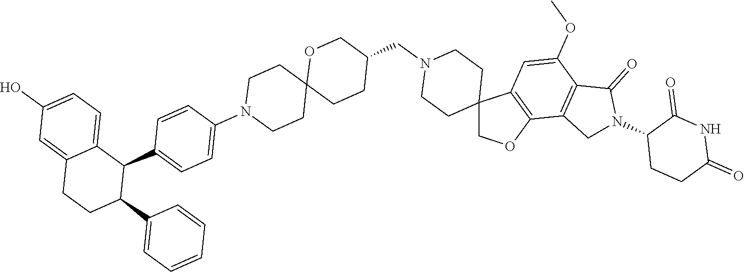 [SMILES: COc1cc2c(c3c1C(=O)N([C@H]1CCC(=O)NC1=O)C3)OCC21CCN(C[C@@H]2CCC3(CCN(c4ccc([C@@H]5c6ccc(O)cc6CC[C@@H]5c5ccccc5)cc4)CC3)OC2)CC1] | (S)-3-(1′-(((S)-9-(4-((1R,2S)- 6-hydroxy-2-phenyl-1,2,3,4- tetrahydronaphthalen-1- yl)phenyl)-1-oxa-9- azaspiro[5.5]undecan-3- yl)methyl)-5-methoxy-6-oxo- 6,8-dihydro-2H,7H- spiro[furo[2,3-e]isoindole- 3,4′-piperidin]-7- yl)piperidine-2,6-dione |

59. A pharmaceutical composition comprising the compound of any one of claims 1-58, and a pharmaceutically acceptable excipient.

60. A method of degrading an estrogen receptor protein in a patient or biological sample comprising administering to the patient a compound of any one of claims 1-58 or contacting the biological sample with a compound of any one of claims 1-58.

61. Use of a compound of any one of claims 1-58 in the manufacture of a medicament for degrading an estrogen receptor protein in a patient or biological sample.

62. A compound of any one of claims 1-58 for use in degrading an estrogen receptor protein in a patient or biological sample.

63. A method of treating a disease or disorder comprising administering to a patient in need thereof a compound of any one of claims 1-58.

64. Use of a compound of any one of claims 1-58 in the manufacture of a medicament for treating a disease or disorder.

65. A compound of any one of claims 1-58 for use in treating a disease or disorder.

66. The method, use, or compound for use of any one of claims 63-65, wherein the disease or disorder is an estrogen receptor-mediated disease or disorder.

67. The method, use, or compound for use of any one of claims 63-65, wherein the disease or disorder is breast cancer, lung cancer, ovarian cancer, endometrial cancer, prostate cancer, or esophageal cancer.
